(19) United States
(12) Patent Application Publication
(10) Pub. No.: US 2026/0092071 A1
(43) Pub. Date: Apr. 2, 2026

(54) COMPOUNDS AND COMPOSITIONS AS SMARCA2/4 DEGRADERS AND USES THEREOF

(71) Applicants: REGENTS OF THE UNIVERSITY OF MICHIGAN, Ann Arbor, MI (US); Oncopia Therapeutics, Inc. d/b/a SK Life Science Labs, King Of Prussia, PA (US)

(72) Inventors: Shaomeng Wang, Superior Township, MI (US); Lingying Leng, Ann Arbor, MI (US); Lin Yang, Ann Arbor, MI (US); Wenbin Tu, Ann Arbor, MI (US); Liyue Huang, Ann Arbor, MI (US); Mi Wang, Ann Arbor, MI (US); Wei Jiang, Novi, MI (US); Paul Kirchhoff, Saline, MI (US); Guozhang Xu, New York, NY (US); Zhenwu Li, New York, NY (US); Lalgudi Harikrishnan, New York, NY (US); E. Scott Priestley, New York, NY (US); Jelena Tosovic, Ann Arbor, MI (US); Jeanne Stuckey, Fenton, MI (US)

(21) Appl. No.: 19/112,750

(22) PCT Filed: Sep. 22, 2023
(86) PCT No.: PCT/US2023/033458

§ 371 Date: Mar. 18, 2025

Related U.S. Application Data
(60) Provisional application No. 63/409,246, filed on Sep. 23, 2022.
(60) Provisional application No. 63/476,587, filed on Dec. 21, 2022.
(60) Provisional application No. 63/460,726, filed on Apr. 20, 2023.

(51) Int. Cl. C07D 487/10; A61K 31/519; A61K 31/527; A61K 31/5377; A61K 31/541; C07D 471/20; C07D 487/04; C07D 487/20; C07D 491/20; C07D 519/00

(52) U.S. Cl. CPC C07D 487/10; A61K 31/519; A61K 31/527; A61K 31/5377; A61K 31/541; C07D 471/20; C07D 487/04; C07D 487/20; C07D 491/20; C07D 519/00

(57) ABSTRACT

Described herein are compounds of Formula II and their pharmaceutically acceptable salts, solvates, or stereoisomers, as well as their uses (e.g., as SMARCA2 or SMARCA4 degraders).

### RELATED APPLICATIONS

This application claims the benefit of and priority to U.S. Provisional Application No. 63/409,246, filed Sep. 23, 2022; U.S. Provisional Application No. 63/476,587, filed Jan. 9, 2023; and U.S. Provisional Application No. 63/460,726, filed Apr. 20, 2023, the contents of each of which are incorporated herein by reference in their entireties.

### BACKGROUND

One of the most significant findings from the cancer genome profiling is the discovery of frequent mutations in various subunits of the mammalian SWI/SNF (SWItch/Sucrose Non-Fermentable) chromatin remodeling complex. Approximately 20% of human cancers are associated with somatic mutations in subunits of the SWI/SNF complex, a chromatin remodeling complex that influences gene regulation by disrupting histone-DNA contacts (PNAS Feb. 25, 2014. Ill (8) 3128-3133).

SWI/SNF complexes contain either of two closely related and evolutionarily conserved catalytic ATPase subunits: Brahma (BRM/SMARCA2) or Brahma-related gene 1 (BRG1/SMARCA4). They share approximately 75% identity at the protein level. Although BRG1- and BRM-containing complexes show some redundancy, they may function distinctively. In human cancer, BRG1 seems to be one of the most frequently mutated subunit genes, whereas the BRM gene is rarely mutated. BRG1/SMARCA4 mutations occurring in 10-15% of lung adenocarcinomas. BRM/SMARCA2, is essential for the growth of tumor cells that harbor loss of function mutations in BRG1/SMARCA4. Depletion of BRM in BRG1-deficient cancer cells leads to a cell cycle arrest, induction of senescence, and increased levels of global H3K9me31.

In some tumor types, mutations within the SWI/SNF complex lead to context specific vulnerabilities such as the requirement of SMARCA2 for survival of tumour cells lacking SMARCA4. This finding of SMARCA2/4 synthetic lethal relationship translates in vivo which emphasizes SMARCA2 as a promising therapeutic target for the treatment SMARCA4-deficient cancers. Moreover, the SMARCA4-deficient patient population generally lacks targetable oncogenes (such as mutant EGFR or ALK translocations), which further emphasizes the potential of developing SMARCA2 inhibitors. Characterization of SMARCA4 function in tumors with high SMARCA4 levels, shows effects on signaling pathways that result in increased proliferation and survival. SMARCA4 knockdown in tumors that show elevated levels known to inhibit proliferation and other cancer cell properties. Studies have also shown that SMARCA4 knock down/modulation increases sensitivity to known chemotherapeutic agents, thereby indicating that SMARCA4 targeting could also be an adjuvant therapy to existing chemotherapeutic approaches.

Contrary to genetic silencing of SMARCA2 leading to potent anti-proliferative activity in SMARCA4-deficient cancer cell lines, PFI-3, a selective cell permeable SMARCA2/4 bromodomain inhibitor capable of binding to SMARCA2 and SMARCA4 bromodomain, pharmacological studies fail to display an antiproliferative phenotype indicating that bromodomain function of SMARCA2/4 is dispensable for tumor cell proliferation, while the catalytic ATPase activity is essential. Therefore, in order to mimic the phenotype achieved by genetic silencing, approaches that lead to reduction or complete elimination of SMARCA2/4 may be needed.

The ubiquitin-proteasome system (UPS) is a major pathway that regulates the levels of intracellular proteins and provides a fine balance between protein synthesis and degradation required for normal maintenance of cellular function, including proliferation, differentiation, and cell death. Ubiquitination is a post-translational modification, where a small protein, ubiquitin, is covalently attached to lysine residues on a substrate protein carried out sequentially by a cascade of enzymatic reactions involving an intimate collaboration between E1 activating, E2 conjugating and E3 ligating enzymes and subsequent degradation of the tagged proteins.

Proteolysis targeting chimeras are the heterobifunctional molecules containing a ligand for a target protein of interest connected via a linker to a ligand for an E3 ubiquitin ligase. Upon such bi-functional molecule-mediated heterodimerization of the two bound proteins, the target protein is ubiquitinated and degraded by the proteasome in cells. Many such bi-functional molecules have been developed to recruit E3 ubiquitin ligases to a variety of substrates using high-affinity ligands for the protein of interest. Proteins effectively degraded using these approaches include RIPK2 and ERRa, BRD4, BRD9, BCR/Abl and Abl and Era. E3 ubiquitin ligases (of which over 600 are known in humans) confer substrate specificity for ubiquitination and are more attractive therapeutic targets than general proteasome inhibitors due to their specificity for certain protein substrates.

### SUMMARY

In certain aspects, the present disclosure provides compounds of Formula II

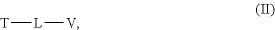

and pharmaceutically acceptable salts, solvates, or stereoisomers thereof, wherein:
T is of Formula II-1

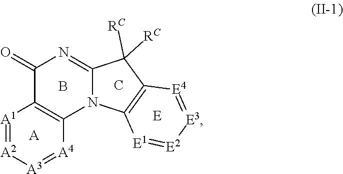

L is of Formula II-2

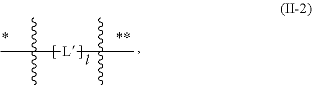

and
   V is of Formula II-3-i, II-3-ii, II-3-iii, II-3-iv, or II-3-v, II-3-vi, II-3-vii, or II-3-viii

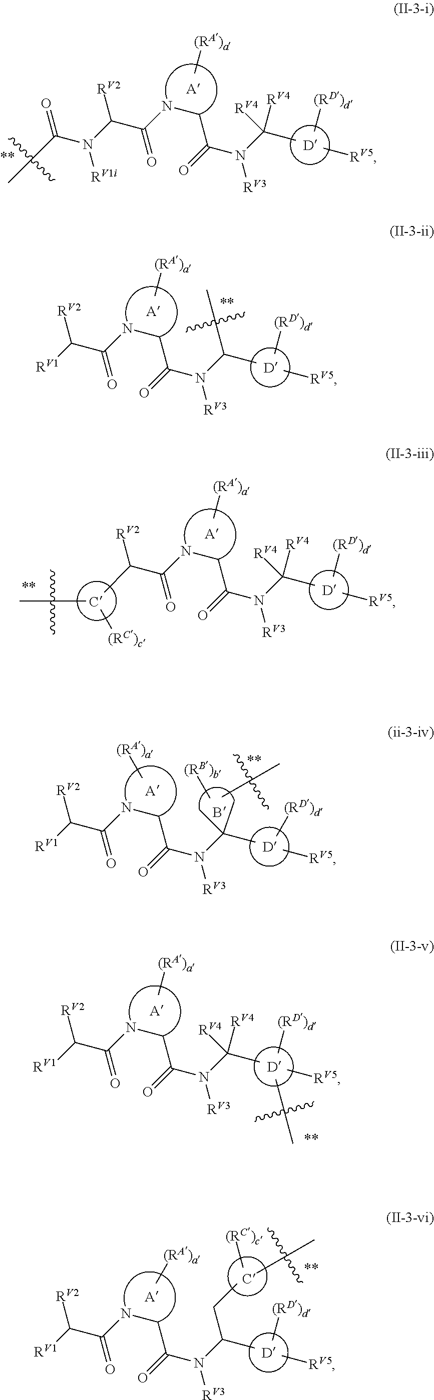

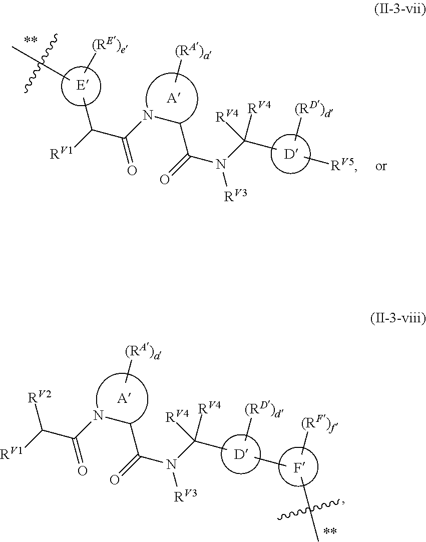

wherein each of the variables in Formulae II-1, II-2, II-3-i, II-3-ii, II-3-iii, II-3-iv, II-3-v, II-3-vi, II-3-vii, and II-3-viii is described, embodied, and exemplified herein.

In certain aspects, the present disclosure provides pharmaceutical compositions comprising a compound disclosed herein, and a pharmaceutically acceptable excipient.

In certain aspects, the present disclosure provides methods of degrading a SMARCA2 or SMARCA4 protein in a subject, comprising administering to the subject a compound disclosed herein.

In certain aspects, the present disclosure provides uses of a compound disclosed herein in the manufacture of a medicament for degrading a SMARCA2 or SMARCA4 protein in a subject.

In certain aspects, the present disclosure provides compounds disclosed herein for use in degrading a SMARCA2 or SMARCA4 protein in a subject.

In certain aspects, the present disclosure provides methods of treating or preventing a disease or disorder in a subject in need thereof, comprising administering to the subject a compound disclosed herein (e.g., in a therapeutically effective amount).

In certain aspects, the present disclosure provides methods of treating a disease or disorder in a subject in need thereof, comprising administering to the subject a compound disclosed herein (e.g., in a therapeutically effective amount).

In certain aspects, the present disclosure provides uses of a compound disclosed herein in the manufacture of a medicament for treating or preventing a disease or disorder in a subject in need thereof.

In certain aspects, the present disclosure provides uses of a compound disclosed herein in the manufacture of a medicament for treating a disease or disorder in a subject in need thereof.

In certain aspects, the present disclosure provides compounds disclosed herein for use in treating or preventing a disease or disorder in a subject in need thereof.

In certain aspects, the present disclosure provides compounds disclosed herein for use in treating a disease or disorder in a subject in need thereof.

### DETAILED DESCRIPTION

The present disclosure relates to compounds and compositions that are useful as SMARCA2 or SMARCA4 protein degraders. The present disclosure also relates to methods of degrading a SMARCA2 or SMARCA4 protein comprising contacting the SMARCA2 or SMARCA4 protein with a SMARCA2 or SMARCA4 protein degrader disclosed herein. The invention also relates to methods of treating a SMARCA2 or SMARCA4 protein-mediated disease or condition in a subject in need thereof by administering (e.g., in a therapeutically effective amount) a SMARCA2 or SMARCA4 protein degrader disclosed herein. The invention further relates to methods of treating a SMARCA2 or SMARCA4 protein-mediated disease or condition in a subject in need thereof, comprising administering (e.g., in a therapeutically effective amount) a pharmaceutical composition comprising an amount of a SMARCA2 or SMARCA4 protein degrader disclosed herein.

### Compounds of the Application

In certain aspects, the present disclosure provides compounds of Formula II

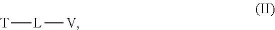

and pharmaceutically acceptable salts, solvates, or stereoisomers thereof, wherein:
T is of Formula II-1

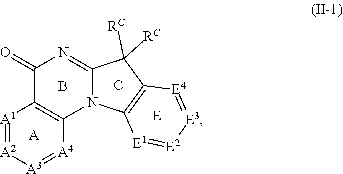

wherein:
A¹ is CR^{A1} or N;
A² is CR^{A2} or N;
A³ is CR^{Z3} or N;
A⁴ is CR^{A4} or N;
R^{A1}, R^{A2}, R^{A3}, and R^{A4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, NR^{c}S(═O)₂NR^{c}R^{d}, —NR^{b}C(═O)NR^{c}R^{a}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two R^{C} together form an oxo; or
two R^{C}, together with the carbon atom to which they are attached, form Ring D

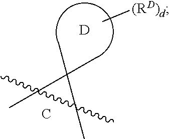

Ring D is C₃₋₁₂ carbocycle or 3- to 12-membered heterocycle;
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 10, as valency permits;
E¹ is CR^{E1} or N;
E² is CR^{E2} or N;
E³ is CR^{E3} or N;
E⁴ is CR^{E4} or N;
R^{E1}, R^{E2}, R^{E3}, and R^{E4} are independently

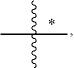

hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
   wherein one of R^{E1}, R^{E2}, R^{E3}, and R^{E4} is

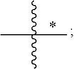

or
one of R^{E2}, R^{E3}, or R^{E4} is

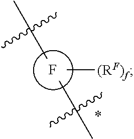

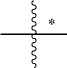

denotes attachment to L;
Ring F is C₃₋₁₂ carbocyclyl, 3- to 12-membered heterocyclyl, C₆₋₁₀ aryl, or 5- to 10-membered heteroaryl;
each R^{F} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
f is an integer selected from 0 to 10, as valency permits,
L is of Formula II-2

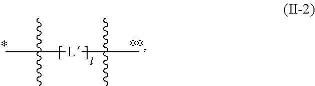

wherein:
* denotes attachment to T and ** denotes attachment to V;
each L′ is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O) N(R^{L′})—, —C(═O)O—, —N(R^{L′})—, —O—, —S—, or —S(═O)₂—, wherein the alkylene, heteroalkylene, alkenylene, alkynylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O) OR′, or —C(═O)NR^{c}R^{d}, wherein the alkyl, alkenyl, alkynyl, carbocyclyl, heterocyclyl, aryl, or heteroaryl is optionally substituted with one or more R^{u}; and
1 is an integer selected from 0 to 5,
   V is of Formula II-3-i, II-3-ii, II-3-iii, II-3-iv, II-3-v, II-3-vi, II-3-vii, or II-3-viii

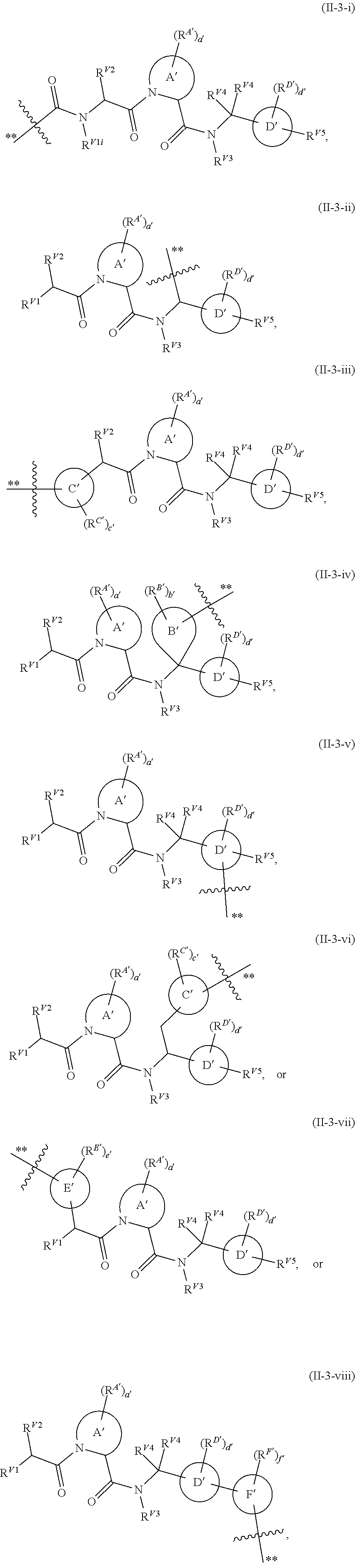

wherein:
** denotes attachment to L;
Ring A′ is 5- to 7-membered heterocycle;
each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a′ is an integer selected from 0 to 10, as valency permits;
Ring B′ is C₃₋₆ carbocycle or 3- to 6-membered heterocycle;
each R^{B′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
b′ is an integer selected from 0 to 10, as valency permits;
Ring C′ is C₆ aryl or 5- to 6-membered heteroaryl;
each R^{C′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
c′ is an integer selected from 0 to 5, as valency permits;
Ring D′ is C₆ aryl or 5- to 6-membered heteroaryl;
each R^{D′} is independently halogen, —CN, —NO₂, —OH, —NH₂, C₁₋₆ alkyl, C₁₋₆alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
R^{D′} and R^{V4}, together with the intervening atoms, form C₃₋₁₂ carbocyclyl, 3- to 12-membered heterocyclyl, C₆₋₁₀ aryl, or 5- to 10-membered heteroaryl, wherein the carbocyclyl, heterocyclyl, aryl, or heteroaryl is optionally substituted with one or more R^{u};
d′ is an integer selected from 0 to 5, as valency permits;
Ring E′ is C₃₋₈ carbocycle or 3- to 8-membered heterocycle;
each R^{E′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
e′ is an integer selected from 0 to 5, as valency permits;
Ring F′ is C₆ aryl or 5- to 6-membered heteroaryl;
each R^{F′} is independently halogen, —CN, —NO₂, —OH, —NH₂, C₁₋₆ alkyl, C₁₋₆ alkoxy, C₁₋₆ alkylamino,
C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; f is an integer selected from 0 to 5, as valency permits;
R^{V1} is —N(R^{V1i})C(═O)R^{V1ii} or 5- to 6-membered heteroaryl optionally substituted with one or more R^{u};
R^{V1i} is hydrogen, C₁₋₆ alkyl, C₃₋₆ carbocyclyl, or 3- to 6-membered heterocyclyl, wherein the carbocyclyl or heterocyclyl is optionally substituted with one or more R^{u};
R^{V1ii} is C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{V1iia};
each R^{V1iia} is independently oxo, halogen, —CN, —NO₂, —OH, —NH₂, C₁₋₆ alkyl, —(C₁₋₆ alkylene)-(3- to 12-membered heterocyclyl), C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkylene, alkoxy, alkylamino, alkenyl, alkynyl, carbocyclyl, heterocyclyl, aryl, or heteroaryl is optionally substituted with one or more R^{u};
R^{V2} is hydrogen, C₁₋₆ alkyl, C₃₋₁₂ carbocyclyl, or 3- to 12-membered heterocyclyl, wherein the alkyl, carbocyclyl, heterocyclyl is optionally substituted with one or more R^{u};
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
each R^{V4} is independently hydrogen, C₂₋₆ alkenyl, C₂₋₆ alkynyl, C₁₋₆ alkyl, C₃₋₆ carbocyclyl, or 3- to 6-membered heterocyclyl, wherein the alkyl, alkenyl, alkynyl, carbocyclyl, or heterocyclyl is optionally substituted with one or more R^{V4a}; or
two R^{V4}, together with the carbon atom to which they are attached, form C₃₋₆ carbocycle or 3- to 6-membered heterocycle, wherein the carbocycle or heterocycle is optionally substituted with one or more R^{u};
each R^{V4a} is independently oxo, halogen, —CN, —NO₂, —OH, —NH₂, C₁₋₆ alkyl, C₁₋₁₂ heteroalkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, heteroalkyl, alkoxy, alkylamino, alkenyl, alkynyl, carbocyclyl, heterocyclyl, aryl, or heteroaryl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, or —C(═O)R^{a}, wherein the alkyl, alkoxy, alkylamino, alkenyl, alkynyl, carbocyclyl, heterocyclyl, aryl, or heteroaryl is optionally substituted with one or more R^{u};
   wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₃₋₆ carbocyclyl, 3- to 6-membered heterocyclyl, C₆ aryl, or 5- to 6-membered heteroaryl, wherein the carbocyclyl, heterocyclyl, aryl, or heteroaryl is optionally substituted with one or more Rz;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, the present disclosure provides compounds of Formula II

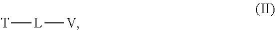

and pharmaceutically acceptable salts, solvates, or stereoisomers thereof, wherein:
T is of Formula II-1

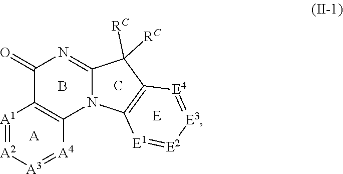

wherein:
A¹ is CR^{A1} or N;
A² is CR^{A2} or N;
A³ is CR^{A3} or N;
A⁴ is CR^{A4} or N;
R^{A1}, R^{A2}, R^{A3}, and R^{A4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two R^{C} together form an oxo; or
two R^{C}, together with the carbon atom to which they are attached, form Ring D

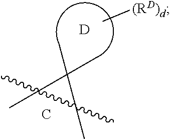

Ring D is C₃₋₁₂ carbocycle or 3- to 12-membered heterocycle;
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 10, as valency permits;
E¹ is CR^{E1} or N;
E² is CR^{E2} or N;
E³ is CR^{E3} or N;
E⁴ is CR^{E4} or N;
one of R^{E2}, R^{E3}, or R^{E4} is

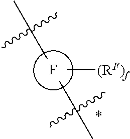

R^{E1}, R^{E2}, and R^{E4}, R^{E1}, R^{E3}, and R^{E4}, or R^{E1}, R^{E2}, and R^{E3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
* denotes attachment to L;
Ring F is C₃₋₁₂ carbocyclyl, 3- to 12-membered heterocyclyl, C₆₋₁₀ aryl, or 5- to 10-membered heteroaryl;
each R^{F} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
f is an integer selected from 0 to 10, as valency permits,
L is of Formula II-2

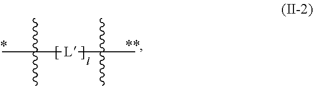

wherein:
* denotes attachment to T and ** denotes attachment to V;
each L′ is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O) N(R^{L′})—, —C(═O)O—, —N(R^{L′})—, —O—, —S—, or —S(═O)₂—, wherein the alkylene, heteroalkylene, alkenylene, alkynylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O) NR^{c}R^{d}, wherein the alkyl, alkenyl, alkynyl, carbocyclyl, heterocyclyl, aryl, or heteroaryl is optionally substituted with one or more R^{u}; and
l is an integer selected from 0 to 5,
   V is of Formula II-3-4, II-3-ii, II-3-iii, II-3-iv, II-3-v, II-3-vi, II-3-vii, or II-3-viii

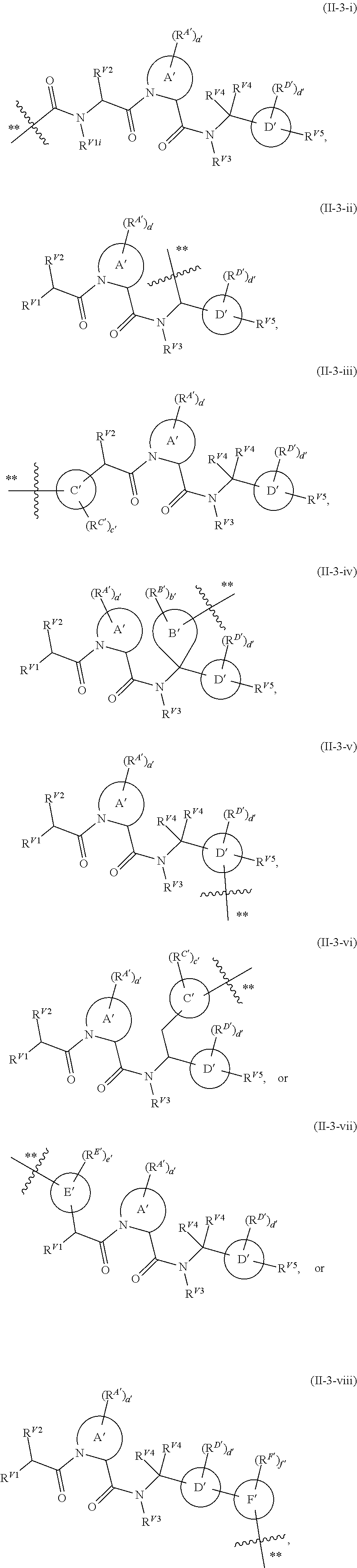

wherein:
** denotes attachment to L;
Ring A′ is 5- to 7-membered heterocycle;
each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₁₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a′ is an integer selected from 0 to 10, as valency permits;
Ring B′ is C₃₋₆ carbocycle or 3- to 6-membered heterocycle;
each R^{B′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
b′ is an integer selected from 0 to 10, as valency permits;
Ring C′ is C₆ aryl or 5- to 6-membered heteroaryl;
each R^{C′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
c′ is an integer selected from 0 to 5, as valency permits;
Ring D′ is C₆ aryl or 5- to 6-membered heteroaryl;
each R^{D′} is independently halogen, —CN, —NO₂, —OH, —NH₂, C₁₋₆ alkyl, C₁₋₆alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
d′ is an integer selected from 0 to 5, as valency permits;
Ring E′ is C₃₋₈ carbocycle or 3- to 8-membered heterocycle;
each R^{E′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
e′ is an integer selected from 0 to 5, as valency permits;
Ring F′ is C₆ aryl or 5- to 6-membered heteroaryl;
each R^{F′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
f′ is an integer selected from 0 to 5, as valency permits;
R^{V1} is —N(R^{V1i})C(═O)R^{V1ii} or 5- to 6-membered heteroaryl optionally substituted with one or more R^{u};
R^{V1i} is hydrogen, C₁₋₆ alkyl, C₃₋₆ carbocyclyl, or 3- to 6-membered heterocyclyl, wherein the carbocyclyl or heterocyclyl is optionally substituted with one or more R^{u};
R^{V1ii} is C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{V1iia}.
each R^{V1ia} is independently oxo, halogen, —CN, —NO₂, —OH, —NH₂, C₁₋₆ alkyl, —(C₁₋₆ alkylene)-(3- to 12-membered heterocyclyl), C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkylene, alkoxy, alkylamino, alkenyl, alkynyl, carbocyclyl, heterocyclyl, aryl, or heteroaryl is optionally substituted with one or more R^{u};
R^{V2} is hydrogen, C₁₋₆ alkyl, C₃₋₁₂ carbocyclyl, or 3- to 12-membered heterocyclyl, wherein the alkyl, carbocyclyl, heterocyclyl is optionally substituted with one or more R^{u};
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
each R^{V4} is independently hydrogen, C₂₋₆ alkenyl, C₂₋₆ alkynyl, C₁₋₆ alkyl, C₃₋₆ carbocyclyl, or 3- to 6-membered heterocyclyl, wherein the alkyl, alkenyl, alkynyl, carbocyclyl, or heterocyclyl is optionally substituted with one or more R^{V4a}; or
two R^{V4}, together with the carbon atom to which they are attached, form C₃₋₆ carbocycle or 3- to 6-membered heterocycle, wherein the carbocycle or heterocycle is optionally substituted with one or more R^{u};
each R^{V4a} is independently oxo, halogen, —CN, —NO₂, —OH, —NH₂, C₁₋₆ alkyl, C₁₋₆ heteroalkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, wherein the alkyl, heteroalkyl, alkoxy, alkylamino, alkenyl, alkynyl, carbocyclyl, heterocyclyl, aryl, or heteroaryl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
   wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₃₋₆ carbocyclyl, 3- to 6-membered heterocyclyl, C₆ aryl, or 5- to 6-membered heteroaryl, wherein the carbocyclyl, heterocyclyl, aryl, or heteroaryl is optionally substituted with one or more R^{z};
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and Rd, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, the compound is a compound of Formula II

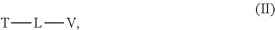

and pharmaceutically acceptable salts, solvates, or stereoisomers thereof, wherein:
T is of Formula II-1

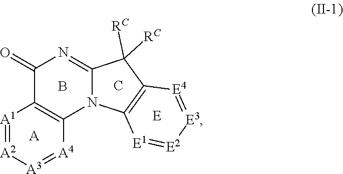

wherein:
A¹ is CR^{A1} or N;
A² is CR^{A2} or N;
A³ is CR^{A3} or N;
A⁴ is CR^{A4} or N;
R^{A1}, R^{A2}, R^{A3}, and R^{A4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{a} S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two R^{C} together form an oxo; or
two R^{C}, together with the carbon atom to which they are attached, form Ring D

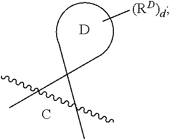

Ring D is C₃₋₁₂ carbocycle or 3- to 12-membered heterocycle;
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 10, as valency permits;
E¹ is CR^{E1} or N;
E² is CR^{E2} or N;
E³ is CR^{E3} or N;
E⁴ is CR^{E4} or N;
one of R^{E2}, R^{E3}, or R^{E4} is

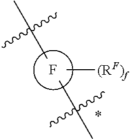

R^{E1}, R^{E2}, and R^{E4}, R^{E1}, R^{E3}, and R^{E4}, or R^{E1}, R^{E2}, and R^{E3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O) OR′, or —C(═O) NR^{c}R^{d}, wherein the alkyl, alkoxy, alkylamino, alkenyl, alkynyl, carbocyclyl, heterocyclyl, aryl, or heteroaryl is optionally substituted with one or more R^{u};
* denotes attachment to L;
Ring F is C₃₋₁₂ carbocyclyl or 3- to 12-membered heterocyclyl;
each R^{F} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
f is an integer selected from 0 to 10, as valency permits,
L is of Formula II-2

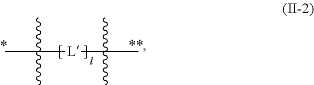

wherein:
* denotes attachment to T and ** denotes attachment to V;
each L′ is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O) N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O) NR^{c}R^{d}, wherein the alkyl, alkenyl, alkynyl, carbocyclyl, heterocyclyl, aryl, or heteroaryl is optionally substituted with one or more R^{u}; and
l is an integer selected from 0 to 5,

### V is of Formula II-3-i, II-3-ii, II-3-iii, II-3-iv, II-3-v, or II-3-vi

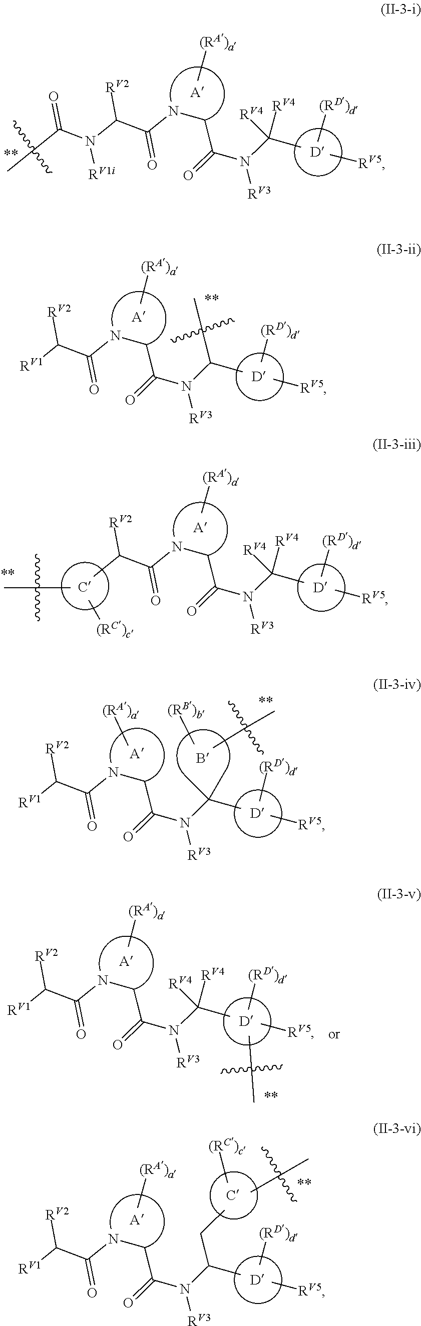

wherein:
* denotes attachment to L;
Ring A′ is 5- to 7-membered heterocycle;
each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a′ is an integer selected from 0 to 10, as valency permits;
Ring B′ is C₃₋₆ carbocycle or 3- to 6-membered heterocycle;
each R^{B′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
b′ is an integer selected from 0 to 10, as valency permits;
Ring C′ is C₆ aryl or 5- to 6-membered heteroaryl;
each R^{C′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
c′ is an integer selected from 0 to 5, as valency permits;
Ring D′ is C₆ aryl or 5- to 6-membered heteroaryl;
each R^{D′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
d′ is an integer selected from 0 to 5, as valency permits;
R^{V1} is —N(R^{V1i})C(═O)R^{V1ii} or 5- to 6-membered heteroaryl optionally substituted with one or more R^{u};
R^{V1i} is hydrogen, C₁₋₆ alkyl, C₃₋₆ carbocyclyl, or 3- to 6-membered heterocyclyl, wherein the carbocyclyl or heterocyclyl is optionally substituted with one or more R^{u};
R^{V1ii} is C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{V1iia},
each R^{V1iia} is independently oxo, halogen, —CN, —NO₂, —OH, —NH₂, C₁₋₆ alkyl, —(C₁₋₆ alkylene)-(3- to 12-membered heterocyclyl), C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkylene, alkoxy, alkylamino, alkenyl, alkynyl, carbocyclyl, heterocyclyl, aryl, or heteroaryl is optionally substituted with one or more R^{u};
R^{V2} is hydrogen or C₁₋₆ alkyl optionally substituted with one or more R^{u};
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O) OR′, or —C(═O)NR^{c}R^{d}; wherein the alkyl, carbocyclyl, or heterocyclyl is optionally substituted with one or more R^{u};
each R^{V4} is independently hydrogen, C₁₋₆ alkyl, C₃₋₆ carbocyclyl, or 3- to 6-membered heterocyclyl, wherein the alkyl, carbocyclyl, or heterocyclyl is optionally substituted with one or more R^{V4a}; or
two R^{V4}, together with the carbon atom to which they are attached, form C₃₋₆ carbocycle or 3- to 6-membered heterocycle, wherein the carbocycle or heterocycle is optionally substituted with one or more R^{u};
each R^{V4a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
   wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₃₋₆ carbocyclyl, 3- to 6-membered heterocyclyl, C₆ aryl, or 5- to 6-membered heteroaryl, wherein the carbocyclyl, heterocyclyl, aryl, or heteroaryl is optionally substituted with one or more R^{z};
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and Rd, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, T is of Formula II-1

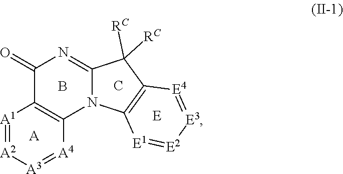

wherein:
A¹ is CR^{A1} or N;
A² is CR^{A2} or N;
A³ is CR^{A3} or N;
A⁴ is CR^{A4} or N;
R^{A1}, R^{A2}, R^{A3}, and R^{A4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R², —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; two R^{C} together form an oxo; or
two R^{C}, together with the carbon atom to which they are attached, form Ring D

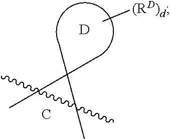

Ring D is C₃₋₁₂ carbocycle or 3- to 12-membered heterocycle;
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 10, as valency permits;
E¹ is CR^{E1} or N;
E² is CR^{E2} or N;
E³ is CRF3 or N;
E⁴ is CR^{E4} or N;
R^{E1}, R^{E2}, R^{E3}, and R^{E4} are independently

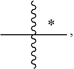

hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R², —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
wherein one of R^{E1}, R^{E2}, R^{E3}, and R^{E4} is

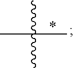

or
one of R^{E2}, R^{E3}, or R^{E4} is

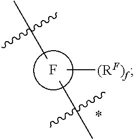

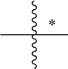

denotes attachment to L;
Ring F is C₃₋₁₂ carbocyclyl, 3- to 12-membered heterocyclyl, C₆₋₁₀ aryl, or 5- to 10-membered heteroaryl;
each R^{F} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
f is an integer selected from 0 to 10, as valency permits.

In certain embodiments, T is of Formula II-1

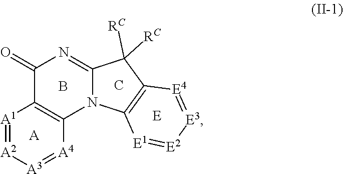

wherein:
A¹ is CR^{A1} or N;
A² is CR^{A2} or N;
A³ is CR^{Z3} or N;
A⁴ is CR^{A4} or N;
R^{A1}, R^{A2}, R^{A3}, and R^{A4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two R^{C} together form an oxo; or
two R^{C}, together with the carbon atom to which they are attached, form Ring D

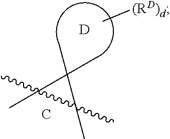

Ring D is C₃₋₁₂ carbocycle or 3- to 12-membered heterocycle;
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR′, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 10, as valency permits;
E¹ is CR^{E1} or N;
E² is CR^{E2} or N;
E³ is CR^{E3} or N;
E⁴ is CR^{E4} or N;
one of R^{E2}, R^{E3}, or R^{E4} is

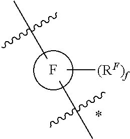

R^{E1}, R^{E2}, and R^{E4}, R^{E1}, R^{E3}, and R^{E4}, or R^{E1}, R^{E2}, and R^{E3} are hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
* denotes attachment to L;
Ring F is C₃₋₁₂ carbocyclyl, 3- to 12-membered heterocyclyl, C₆₋₁₀ aryl, or 5- to 10-membered heteroaryl;
each R^{F} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR⁵, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
f is an integer selected from 0 to 10, as valency permits.

In certain embodiments, T is of Formula II-1

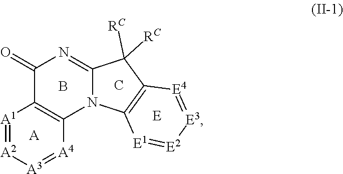

wherein:
A¹ is CR^{A1} or N;
A² is CR^{A2} or N;
A³ is CR^{A3} or N;
A⁴ is CR^{A4} or N;
R^{A1}, R^{A2}, R^{A3}, and R^{A4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two R^{C} together form an oxo; or
two R^{C}, together with the carbon atom to which they are attached, form Ring D

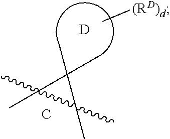

Ring D is C₃₋₁₂ carbocycle or 3- to 12-membered heterocycle;
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR′, —OS(═O)₂NR^{c}R^{d}, —OC(═O)R^{a}, —OC(═O)OR^{b}, —OC(═O)NR^{c}R^{d}, —C(═O)R^{a}, —C(═O) OR′, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 10, as valency permits;
E¹ is CR^{E1} or N;
E² is CR^{E2} or N;
E³ is CR^{E3} or N;
E⁴ is CR^{E4} or N;
R^{E2} or R^{E3} is

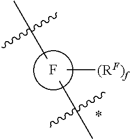

R^{E1}, R^{E2}, and R^{E4} or R^{E1}, R^{E3}, and R^{E4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR′, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
* denotes attachment to L;
Ring F is C₃₋₁₂ carbocyclyl or 3- to 12-membered heterocyclyl;
each R^{F} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
f is an integer selected from 0 to 10, as valency permits.

In certain embodiments, T is of Formula II-1-i or II-1-ii

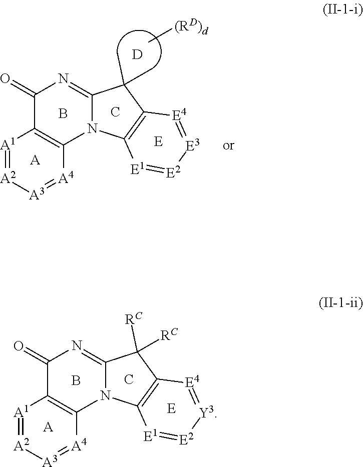

In certain embodiments, T is of Formula II-1-i or II-1-ii

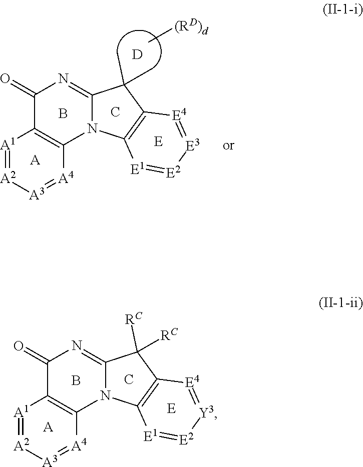

wherein:
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, T is of Formula II-1-i-1, II-1-i-2, II-1-i-3, II-1-ii-1, II-1-ii-2, II-1-ii-3, II-1-iii-1, II-1-iii-2, or II-1-iii-3

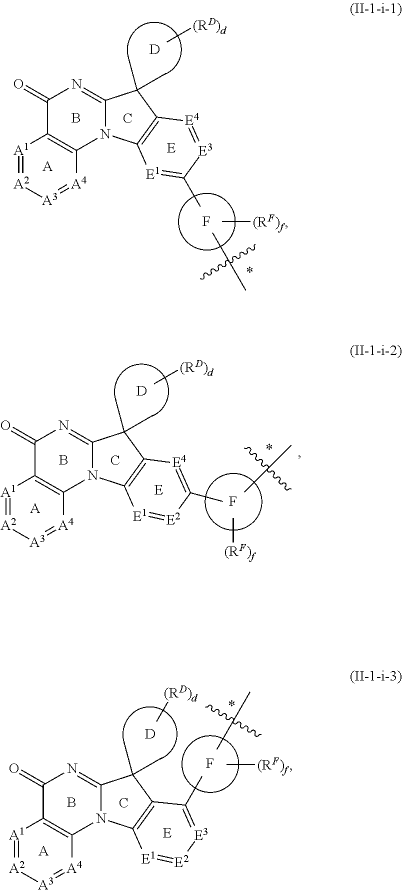

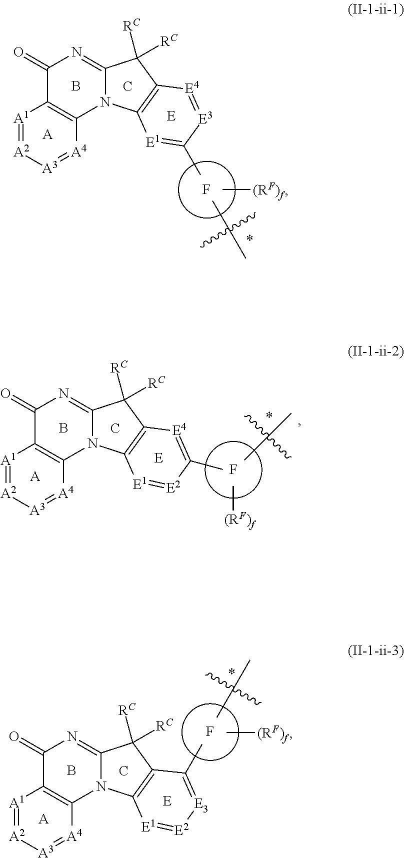

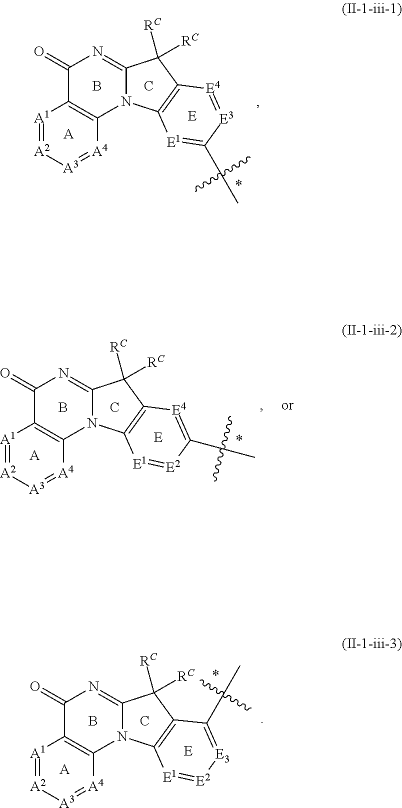

In certain embodiments, T is of Formula II-1-i-1, II-1-i-2, II-1-i-3, II-1-ii-1, II-1-ii-2, II-1-ii-3, II-1-iii-1, II-1-iii-2, or II-1-iii-3

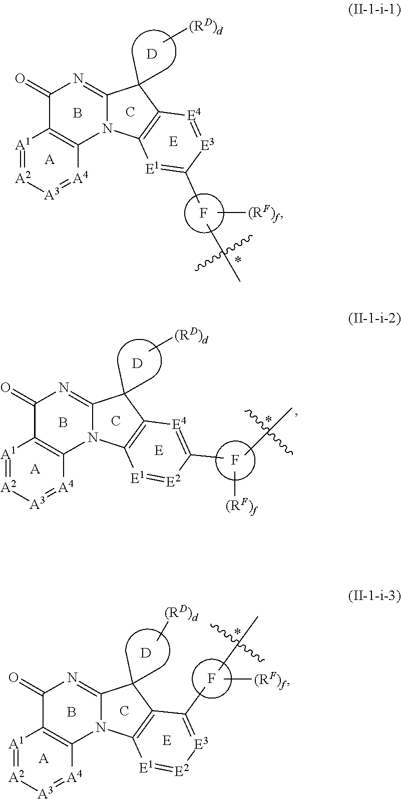

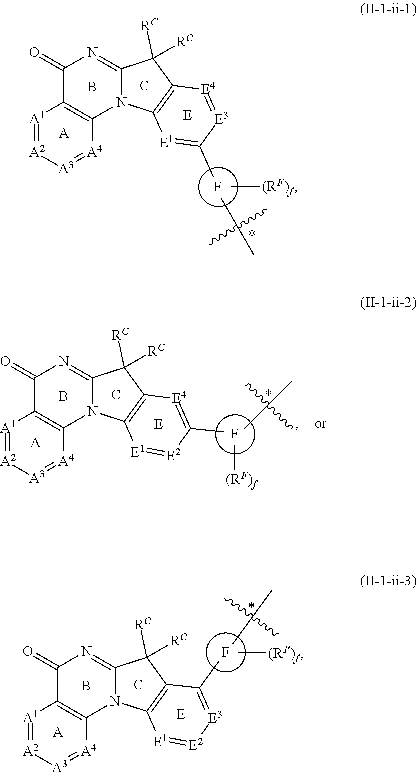

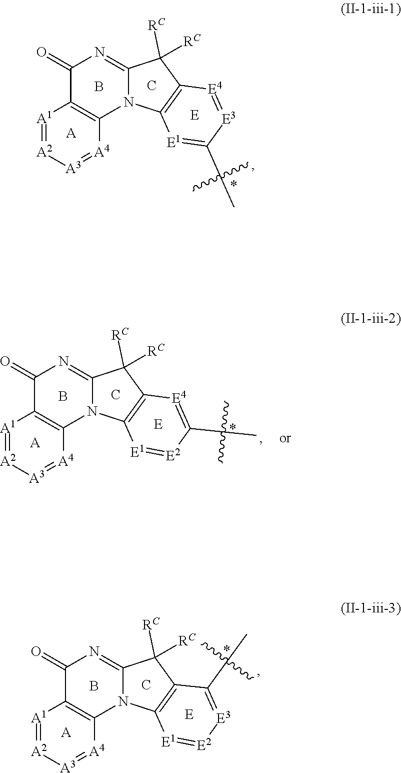

each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, T is of Formula II-1-i-4, II-1-i-5, II-1-i-6, II-1-ii-4, II-1-ii-5, II-1-ii-6, II-1-iii-4, II-1-iii-5, or II-1-iii-6

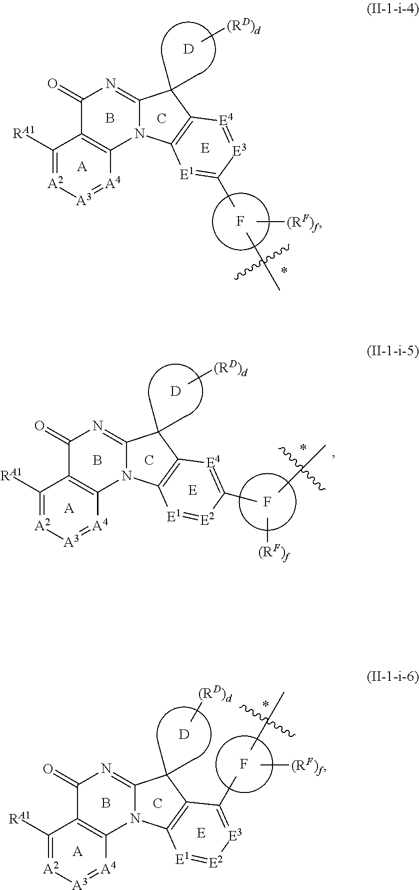

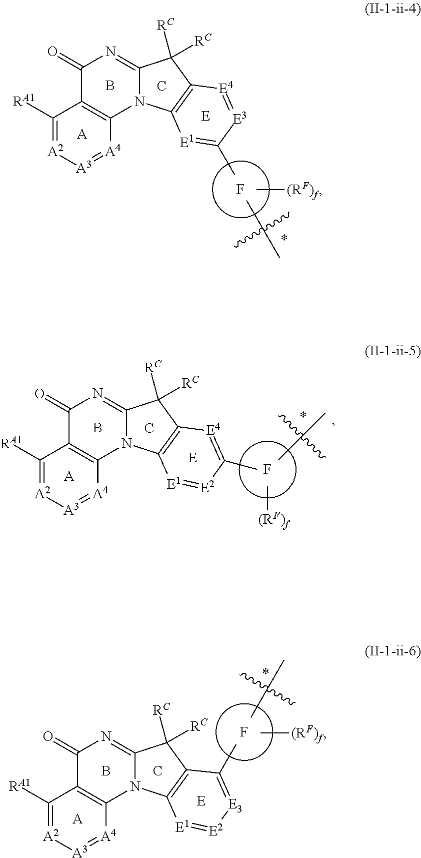

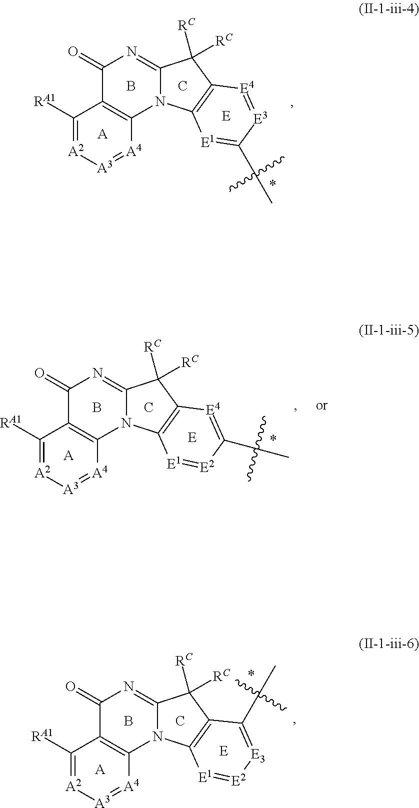

wherein R^{A1} is halogen or hydrogen.

In certain embodiments, T is of Formula II-1-i-4, II-1-i-5, II-1-i-6, II-1-ii-4, II-1-ii-5, II-1-ii-6, II-1-iii-4, II-1-iii-5, or II-1-iii-6

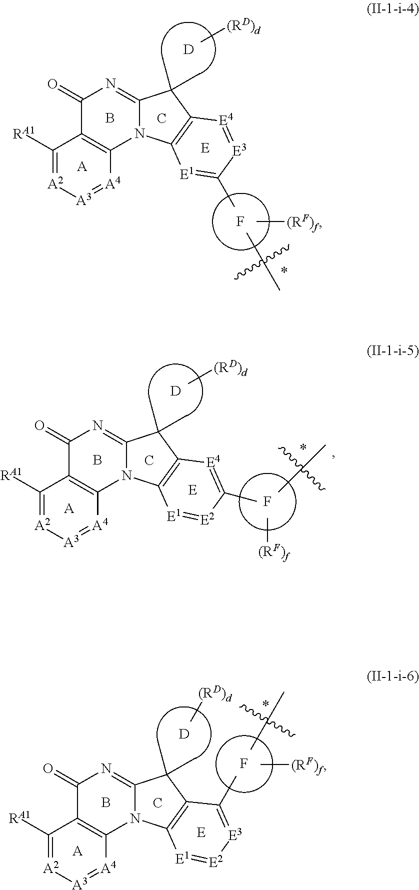

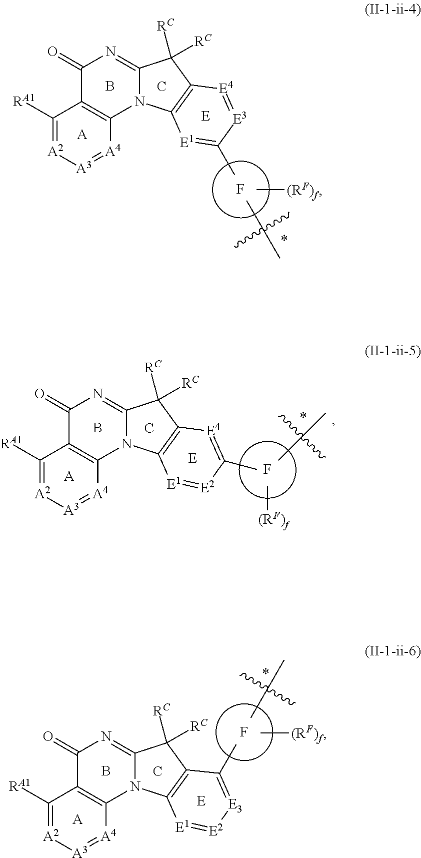

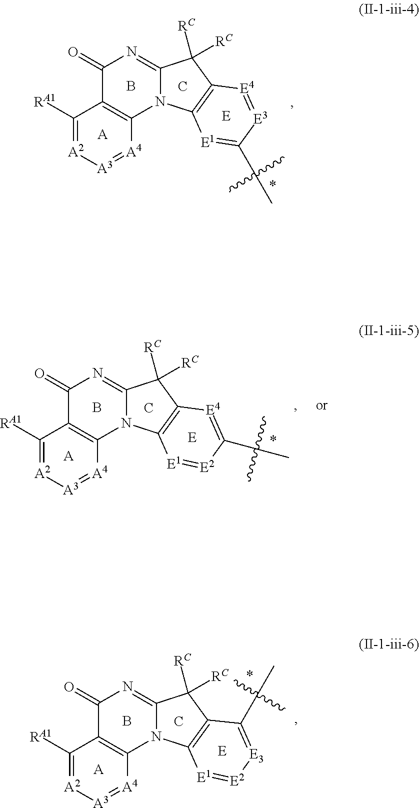

wherein
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}; and
R^{A1} is halogen or hydrogen.

In certain embodiments, A¹ is CR^{A1} or N. In certain embodiments, A¹ is N. In certain embodiments, A¹ is CR^{A1}.

In certain embodiments, A² is CR^{A2} or N. In certain embodiments, A² is N. In certain embodiments, A² is CR^{A2}.

In certain embodiments, A³ is CR^{A3} or N. In certain embodiments, A³ is N. In certain embodiments, A³ is CR^{A3}.

In certain embodiments, A⁴ is CR^{A4} or N. In certain embodiments, A⁴ is N. In certain embodiments, A⁴ is CR^{A4}.

In certain embodiments, none of A¹, A², A³, and A⁴ is N. In certain embodiments, one of A¹, A², A³, and A⁴ is N. In certain embodiments, two of A¹, A², A³, and A⁴ are N. In certain embodiments, three of A¹, A², A³, and A⁴ are N. In certain embodiments, each of A¹, A², A³, and A⁴ is N.

In certain embodiments, R^{A1}, R^{A2}, R^{A3}, and R^{A4}, when applicable, are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), n-propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-i-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, S-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{A1}, R^{A2}, R^{A3}, and R^{A4}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{A1}, R^{A2}, R^{A3}, and R^{A4}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{A1}, R^{A2}, R^{A3}, and R^{A4}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl, is optionally substituted with one or more R^{u}.

In certain embodiments, R^{A1}, R^{A2}, R^{A3}, and R^{A4}, when applicable, are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, RAI is hydrogen or halogen. In certain embodiments, R^{A1} is hydrogen. In certain embodiments, R^{A1} is halogen. In certain embodiments, R^{A2} is hydrogen. In certain embodiments, R^{A3} is hydrogen. In certain embodiments, R^{A4} is hydrogen.

In certain embodiments, each R^{C} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), n-propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{a}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl, is optionally substituted with one or more R^{u}.

In certain embodiments, each R^{C} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, each R^{C} is independently C₁₋₆ alkyl optionally substituted with one or more R^{u}. In certain embodiments, at least one R^{C} is C₁₋₆ alkyl optionally substituted with one or more R^{u}. In certain embodiments, at least one R^{C} is C₁₋₆ haloalkyl optionally substituted with one or more R^{u}. In certain embodiments, each R^{C} is independently C₁₋₆ alkyl.

In certain embodiments, two R^{C} together form an oxo.

In certain embodiments, two R^{C}, together with the carbon atom to which they are attached, form Ring D

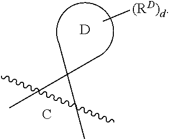

In certain embodiments, Ring D is C₃₋₁₂ carbocycle (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)) or 3- to 12-membered heterocycle (e.g., heterocyclyl comprising one or two 3- to 8-membered rings and 1-5 heteroatoms selected from N, O, and S).

In certain embodiments, Ring D is cyclopentane ring, cyclohexane ring, or tetrahydropyran ring.

In certain embodiments, each R^{D} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), n-propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR′, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl, is optionally substituted with one or more R^{u}.

In certain embodiments, each R^{D} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, d is 0. In certain embodiments, d is 1. In certain embodiments, d is 2. In certain embodiments, d is 3. In certain embodiments, d is 4. In certain embodiments, d is 5. In certain embodiments, d is 6. In certain embodiments, d is 7. In certain embodiments, d is 8. In certain embodiments, d is 9. In certain embodiments, d is 10.

In certain embodiments, E¹ is CR^{E1} or N. In certain embodiments, E¹ is N. In certain embodiments, E¹ is CR^{E1}.

In certain embodiments, E² is CR^{E2} or N. In certain embodiments, E² is N. In certain embodiments, E² is CR^{E2}.

In certain embodiments, E³ is CR^{E3} or N. In certain embodiments, E³ is N. In certain embodiments, E³ is CR^{E3}.

In certain embodiments, E⁴ is CR^{E4} or N. In certain embodiments, E⁴ is N. In certain embodiments, E⁴ is CR^{E4}.

In certain embodiments, none of E¹, E², E³, and E⁴ is N.

In certain embodiments, one of E¹, E², E³, and E⁴ is N.

In certain embodiments, two of E¹, E², E³, and E⁴ are N.

In certain embodiments, three of E¹, E², E³, and E⁴ are N.

In certain embodiments, each of E¹, E², E³, and E⁴ is N.

In certain embodiments, R^{E1}, R^{E2}, R^{E3}, and R^{E4}, when applicable, are independently

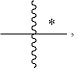

hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), n-propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-i-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, S-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O) NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

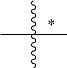

In certain embodiments, R^{E1}, R^{E2}, R^{E3}, and R^{E4}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

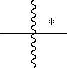

In certain embodiments, R^{E1}, R^{E2}, R^{E3}, and R^{E4}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

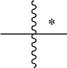

In certain embodiments, R^{E1}, R^{E2}, R^{E3}, and R^{E4}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl, is optionally substituted with one or more R^{u}.

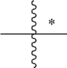

In certain embodiments, R^{E1}, R^{E2}, R^{E3}, and R^{E4}, when applicable, are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, one of R^{E1}, R^{E2}, R^{E3}, and R^{E4} is

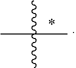

In certain embodiments, R^{E1} is

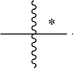

In certain embodiments, R^{E2} is

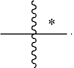

In certain embodiments, R^{E3} is

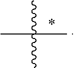

In certain embodiments, R^{E4} is

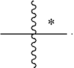

In certain embodiments, R^{E1} is hydrogen. In certain embodiments, R^{E2} is hydrogen. In certain embodiments, R^{E3} is hydrogen. In certain embodiments, R^{E4} is hydrogen.

In certain embodiments, one of R^{E2}, R^{E3}, or R^{E4} is

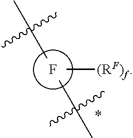

In certain embodiments, Ring F is C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphalenyl), or 5- to 10-membered heteroaryl (e.g., heteroaryl comprising one or two 5- or 6-membered rings and 1-4 heteroatoms selected from N, O, and S).

In certain embodiments, Ring F is 3- to 12-membered heterocyclyl or C₃₋₁₂ carbocyclyl.

In certain embodiments, Ring F is piperidinyl, piperazinyl, 2,7-diazaspiro[3.5]nonanyl, or 3,9-diazaspiro[5.5]undecanyl.

In certain embodiments, Ring F is piperidinyl or piperazinyl.

In certain embodiments, each R^{F} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), n-propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O) NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{F} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{F} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{F} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl, is optionally substituted with one or more R^{u}.

In certain embodiments, each R^{F} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, f is 0. In certain embodiments, f is 1. In certain embodiments, f is 2. In certain embodiments, f is 3. In certain embodiments, f is 4. In certain embodiments, f is 5. In certain embodiments, f is 6. In certain embodiments, f is 7. In certain embodiments, f is 8. In certain embodiments, f is 9. In certain embodiments, f is 10.

V is a residue of a ligand that binds von Hippel-Lidau E³ ubiquitin ligase (VHL ligand). Representative VHL ligands are disclosed in, for example, i) WO 2019/207538, WO 2020/251972, WO 2020/251971, WO 2021/155321, WO2021/086785, WO 2021/133920, and WO 2022/109396; ii) Kofink, C., Trainor, N., Mair, B. et al. A selective and orally bioavailable VHL-recruiting PROTAC achieves SMARCA2 degradation in vivo. Nat Commun 13, 5969 (2022); iii) Cantley, J., Ye, X., Rousseau, E. et al. Selective PROTAC-mediated degradation of SMARCA2 is efficacious in SMARCA4 mutant cancers. Nat Commun 13, 6814 (2022); iv) WO 2022/125804 and CN112979747; and v) Farnaby, W. et al. BAF complex vulnerabilities in cancer demonstrated via structure-based PROTAC design. Nat. Chem. Biol. 15, 672-680 (2019).

In certain embodiments, V is of Formula II-3-i, II-3-ii, II-3-iii, II-3-iv, II-3-v, II-3-vi, II-3-vii, or II-3-viii

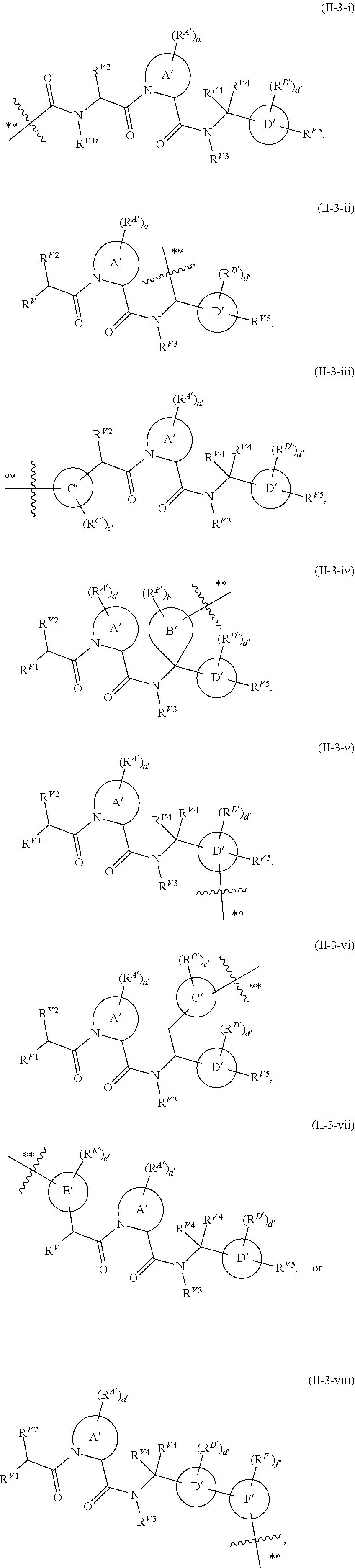

wherein:
** denotes attachment to L;
Ring A′ is 5- to 7-membered heterocycle;
each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a′ is an integer selected from 0 to 10, as valency permits;
Ring B′ is C₃₋₆ carbocycle or 3- to 6-membered heterocycle;
each R^{B′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
b′ is an integer selected from 0 to 10, as valency permits;
Ring C′ is C₆ aryl or 5- to 6-membered heteroaryl;
each R^{C′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; c′ is an integer selected from 0 to 5, as valency permits;
Ring D′ is C₆ aryl or 5- to 6-membered heteroaryl;
each R^{D′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or R^{D′} and R^{V4}, together with the atoms to which they are bonded, form C₃₋₁₂ carbocyclyl, 3- to 12-membered heterocyclyl, C₆₋₁₀ aryl, or 5- to 10-membered heteroaryl, wherein the carbocyclyl, heterocyclyl, aryl, or heteroaryl is optionally substituted with one or more R^{u};
d′ is an integer selected from 0 to 5, as valency permits; Ring E′ is C₃₋₈ carbocycle or 3- to 8-membered heterocycle;
each R^{E′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
e′ is an integer selected from 0 to 5, as valency permits;
Ring F′ is C₆ aryl or 5- to 6-membered heteroaryl;
each R^{F′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; f′ is an integer selected from 0 to 5, as valency permits;
R^{V1} is —N(R^{V1i})C(═O)R^{V1ii} or 5- to 6-membered heteroaryl optionally substituted with one or more R^{u};
R^{V1i} is hydrogen, C₁₋₆ alkyl, C₃₋₆ carbocyclyl, or 3- to 6-membered heterocyclyl, wherein the alkyl, carbocyclyl, or heterocyclyl is optionally substituted with one or more R^{u};
R^{V1ii} is C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{V1iia}.
each R^{V1ia} is independently oxo, halogen, —CN, —NO₂, —OH, —NH₂, C₁₋₆ alkyl, —(C₁₋₆ alkylene)-(3- to 12-membered heterocyclyl), C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkylene, alkoxy, alkylamino, alkenyl, alkynyl, carbocyclyl, heterocyclyl, aryl, or heteroaryl is optionally substituted with one or more R^{u};
R^{V2} is hydrogen, C₁₋₆ alkyl, C₃₋₁₂ carbocyclyl, or 3- to 12-membered heterocyclyl, wherein the alkyl, carbocyclyl, heterocyclyl is optionally substituted with one or more R^{u};
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
each R^{V4} is independently hydrogen, C₁₋₆ alkyl, C₂₋₆ alkenyl, C₂₋₆ alkynyl, C₃₋₆ carbocyclyl, or 3- to 6-membered heterocyclyl, wherein the alkyl, alkenyl, alkynyl, carbocyclyl, or heterocyclyl is optionally substituted with one or more R^{V4a}, or
two R^{V4}, together with the carbon atom to which they are attached, form C₃₋₆ carbocycle or 3- to 6-membered heterocycle, wherein the carbocycle or heterocycle is optionally substituted with one or more R^{u};
each R^{V4a} is independently oxo, halogen, —CN, —NO₂, —OH, —NH₂, C₁₋₆ alkyl, C₁₋₁₂ heteroalkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, heteroalkyl, alkoxy, alkylamino, alkenyl, alkynyl, carbocyclyl, heterocyclyl, aryl, or heteroaryl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, or —C(═O)R^{a}, wherein the alkyl, alkoxy, alkylamino, alkenyl, alkynyl, carbocyclyl, heterocyclyl, aryl, or heteroaryl is optionally substituted with one or more R^{u}.

In certain embodiments, V is of Formula II-3-i, II-3-ii, II-3-iii, II-3-iv, II-3-v, II-3-vi, II-3-vii, or II-3-viii

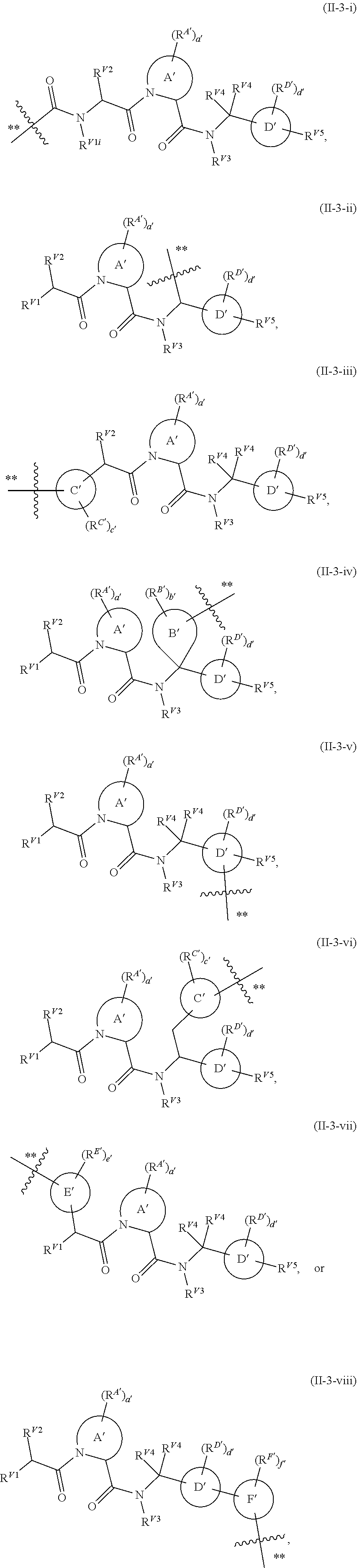

** denotes attachment to L;
Ring A′ is 5- to 7-membered heterocycle;
each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{a};
a′ is an integer selected from 0 to 10, as valency permits;
Ring B′ is C₃₋₆ carbocycle or 3- to 6-membered heterocycle;
each R^{B′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
b′ is an integer selected from 0 to 10, as valency permits;
Ring C′ is C₆ aryl or 5- to 6-membered heteroaryl;
each R^{C′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
c′ is an integer selected from 0 to 5, as valency permits;
Ring D′ is C₆ aryl or 5- to 6-membered heteroaryl;
each R^{D′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
d′ is an integer selected from 0 to 5, as valency permits;
Ring E′ is C₃₋₈ carbocycle or 3- to 8-membered heterocycle;
each R^{E′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
e′ is an integer selected from 0 to 5, as valency permits;
Ring F′ is C₆ aryl or 5- to 6-membered heteroaryl;
each R^{F′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; f is an integer selected from 0 to 5, as valency permits;
R^{V1} is —N(R^{V1i})C(═O)R^{V1ii} or 5- to 6-membered heteroaryl optionally substituted with one or more R^{u};
R^{V1i} is hydrogen, C₁₋₆ alkyl, C₃₋₆ carbocyclyl, or 3- to 6-membered heterocyclyl, wherein the carbocyclyl or heterocyclyl is optionally substituted with one or more R^{u};
R^{V1ii} is C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{V1iia},
each R^{V1ia} is independently oxo, halogen, —CN, —NO₂, —OH, —NH₂, C₁₋₆ alkyl, —(C₁₋₆ alkylene)-(3- to 12-membered heterocyclyl), C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkylene, alkoxy, alkylamino, alkenyl, alkynyl, carbocyclyl, heterocyclyl, aryl, or heteroaryl is optionally substituted with one or more R^{u};
R^{V2} is hydrogen, C₁₋₆ alkyl, C₃₋₁₂ carbocyclyl, or 3- to 12-membered heterocyclyl, wherein the alkyl, carbocyclyl, heterocyclyl is optionally substituted with one or more R^{u};
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
each R^{V4} is independently hydrogen, C₂₋₆ alkenyl, C₂₋₆ alkynyl, C₁₋₆ alkyl, C₃₋₆ carbocyclyl, or 3- to 6-membered heterocyclyl, wherein the alkyl, alkenyl, alkynyl, carbocyclyl, or heterocyclyl is optionally substituted with one or more R^{V4a}; or
two R^{V4}, together with the carbon atom to which they are attached, form C₃₋₆ carbocycle or 3- to 6-membered heterocycle, wherein the carbocycle or heterocycle is optionally substituted with one or more R^{u};
each R^{V4a} is independently oxo, halogen, —CN, —NO₂, —OH, —NH₂, C₁₋₆ alkyl, C₁₋₆ heteroalkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, heteroalkyl, alkoxy, alkylamino, alkenyl, alkynyl, carbocyclyl, heterocyclyl, aryl, or heteroaryl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, V is of Formula II-3-i, II-3-ii, II-3-iii, II-3-iv, II-3-v, or II-3-vi

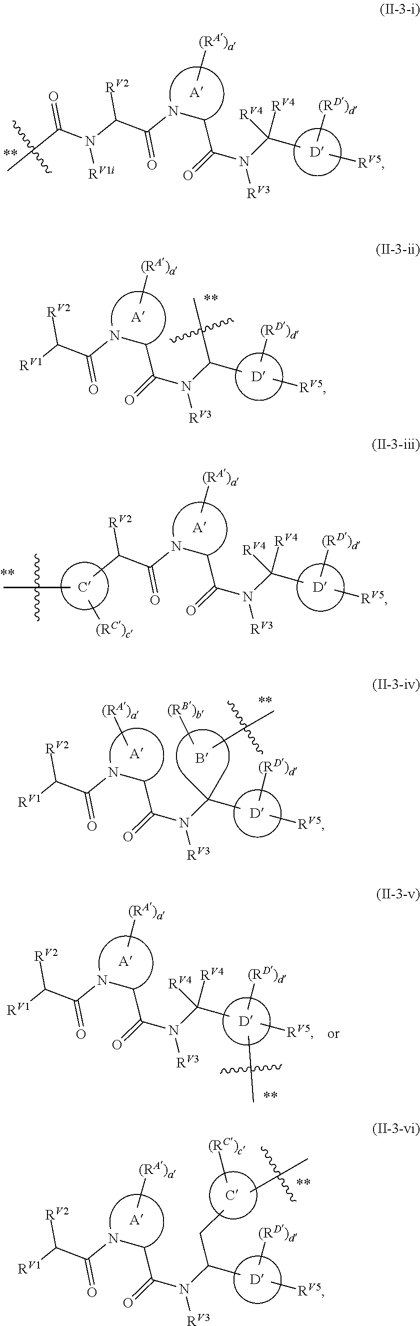

** denotes attachment to L;
Ring A′ is 5- to 7-membered heterocycle;
each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a′ is an integer selected from 0 to 10, as valency permits;
Ring B′ is C₃₋₆ carbocycle or 3- to 6-membered heterocycle;
each R^{B′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
b′ an integer selected from 0 to 10, as valency permits;
Ring C′ is C₆ aryl or 5- to 6-membered heteroaryl;
each R^{C′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
c′ is an integer selected from 0 to 5, as valency permits;
Ring D′ is C₆ aryl or 5- to 6-membered heteroaryl;
each R^{D′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
d′ is an integer selected from 0 to 5, as valency permits;
R^{V1} is —N(R^{V1i})C(═O)R^{V1ii} or 5- to 6-membered heteroaryl optionally substituted with one or more R^{u};
R^{V1i} is hydrogen, C₁₋₆ alkyl, C₃₋₆ carbocyclyl, or 3- to 6-membered heterocyclyl, wherein the carbocyclyl or heterocyclyl is optionally substituted with one or more R^{u};
R^{V1ii} is C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{V1iia}.
each R^{V1ia} is independently oxo, halogen, —CN, —NO₂, —OH, —NH₂, C₁₋₆ alkyl, —(C₁₋₆ alkylene)-(3- to 12-membered heterocyclyl), C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
R^{V2} is hydrogen or C₁₋₆ alkyl optionally substituted with one or more R^{u};
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O) NR^{c}R^{d}; wherein the alkyl, carbocyclyl, or heterocyclyl is optionally substituted with one or more R^{u};
each R^{V4} is independently hydrogen, C₁₋₆ alkyl, C₃₋₆ carbocyclyl, or 3- to 6-membered heterocyclyl, wherein the alkyl, carbocyclyl, or heterocyclyl is optionally substituted with one or more R^{V4a}, or
two R^{V4}, together with the carbon atom to which they are attached, form C₃₋₆ carbocycle or 3- to 6-membered heterocycle, wherein the carbocycle or heterocycle is optionally substituted with one or more R^{u};
each R^{V4a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, V is of Formula II-3-i

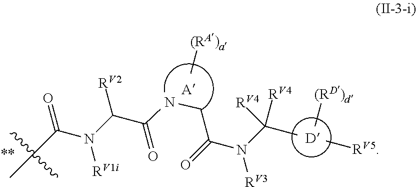

In certain embodiments, V is of Formula II-3-3

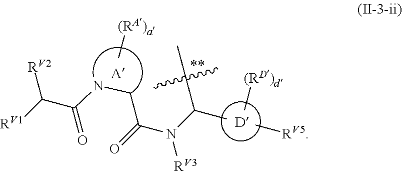

In certain embodiments, V is of Formula II-3-M

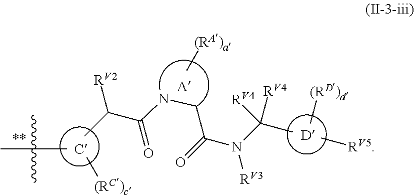

In certain embodiments, V is of Formula II-3-iv

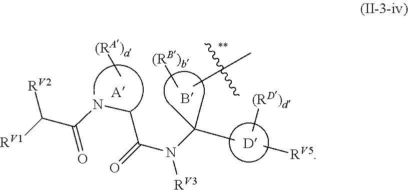

In certain embodiments, V is of Formula II-3-v

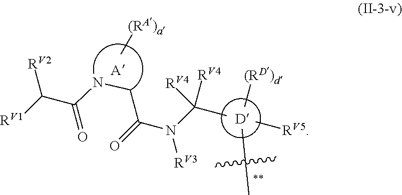

In certain embodiments, V is of Formula II-3-vi

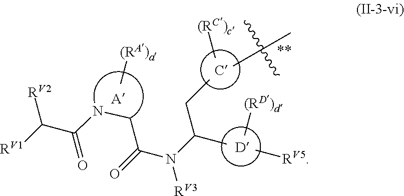

In certain embodiments, V is of Formula II-3-vii

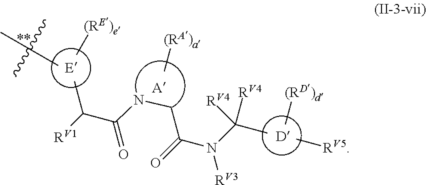

In certain embodiments, V is of Formula II-3-viii

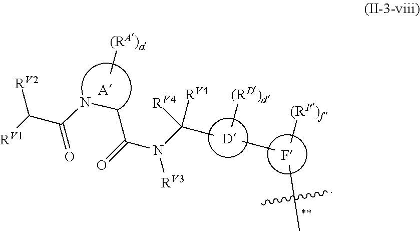

In certain embodiments, Ring A′ is 5- to 7-membered heterocycle (e.g., heterocyclyl comprising one 5- to 7-membered ring and 1-3 heteroatoms selected from N, O, and S). In certain embodiments, Ring A′ is 5-membered heterocycle. In certain embodiments, Ring A′ is 6-membered heterocycle. In certain embodiments, Ring A′ is 7-membered heterocycle.

In certain embodiments,

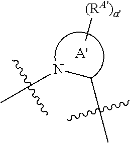

is

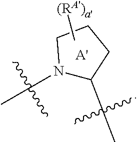

In certain embodiments, a′ is 0. In certain embodiments, a′ is 1. In certain embodiments, a′ is 2. In certain embodiments, a′ is 3. In certain embodiments, a′ is 4, as valency permits. In certain embodiments, a′ is 5, as valency permits. In certain embodiments, a′ is 6, as valency permits. In certain embodiments, a′ is 7, as valency permits. In certain embodiments, a′ is 8, as valency permits. In certain embodiments, a′ is 9, as valency permits. In certain embodiments, a′ is 10, as valency permits.

In certain embodiments, each R^{A′} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{A′} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, at least one R^{A′} is —OH.

In certain embodiments, a′ is 1 or 2, and at least one of R^{A′} is —OH.

In certain embodiments,

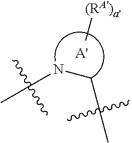

is

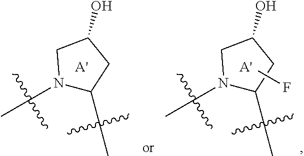

In certain embodiments, Ring B′ is C₃₋₅ carbocycle (e.g., cyclopropyl (C₃), cyclopropenyl (C₃), cyclobutyl (C₄), cyclobutenyl (C₄), cyclopentyl (C₅), or cyclopentenyl (C₅)) or 3- to 5-membered heterocycle (e.g., heterocycle comprising one 3- to 5-membered ring and 1 or 2 heteroatoms selected from N, O, and S). In certain embodiments, Ring B′ is C₃₋₅ carbocycle. In certain embodiments, Ring B′ is 3- to 5-membered heterocycle.

In certain embodiments, each R^{B′} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{B′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{B′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{B′} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, b′ is 0. In certain embodiments, b′ is 1. In certain embodiments, b′ is 2. In certain embodiments, b′ is 3. In certain embodiments, b′ is 4, as valency permits. In certain embodiments, b′ is 5, as valency permits. In certain embodiments, b′ is 6, as valency permits. In certain embodiments, b′ is 7, as valency permits. In certain embodiments, b′ is 8, as valency permits. In certain embodiments, b′ is 9, as valency permits. In certain embodiments, b′ is 10, as valency permits.

In certain embodiments, Ring C′ is C₆ aryl (i.e., phenyl) or 5- to 6-membered heteroaryl (e.g., heteroaryl comprising one 5- or 6-membered ring and 1-4 heteroatoms selected from N, O, and S). In certain embodiments, Ring C′ is C₆ aryl (i.e., phenyl). In certain embodiments, Ring C is 5-membered heteroaryl. In certain embodiments, Ring C is 6-membered heteroaryl.

In certain embodiments, Ring C′ is 5-membered heteroaryl comprising one nitrogen atom. In certain embodiments, Ring C′ is 5-membered heteroaryl comprising two nitrogen atoms. In certain embodiments, Ring C′ is 5-membered heteroaryl comprising three nitrogen atoms. In certain embodiments, Ring C′ is 5-membered heteroaryl comprising four nitrogen atoms.

In certain embodiments, Ring C′ is 5-membered heteroaryl comprising at least one nitrogen atom. In certain embodiments, Ring C′ is 5-membered heteroaryl comprising at least two nitrogen atoms. In certain embodiments, Ring C′ is 5-membered heteroaryl comprising at least three nitrogen atoms. In certain embodiments, Ring C′ is 5-membered heteroaryl comprising at least four nitrogen atoms.

In certain embodiments, Ring C′ is 6-membered heteroaryl comprising one nitrogen atom. In certain embodiments, Ring C′ is 6-membered heteroaryl comprising two nitrogen atoms. In certain embodiments, Ring C′ is 6-membered heteroaryl comprising three nitrogen atoms. In certain embodiments, Ring C′ is 6-membered heteroaryl comprising four nitrogen atoms.

In certain embodiments, Ring C′ is 6-membered heteroaryl comprising at least one nitrogen atom. In certain embodiments, Ring C′ is 6-membered heteroaryl comprising at least two nitrogen atoms. In certain embodiments, Ring C′ is 6-membered heteroaryl comprising at least three nitrogen atoms. In certain embodiments, Ring C′ is 6-membered heteroaryl comprising at least four nitrogen atoms.

In certain embodiments, Ring C′ is 5-membered heteroaryl comprising at least one nitrogen atom.

In certain embodiments, Ring C′ is isoxazole. In certain embodiments, Ring C′ is triazole. In certain embodiments, Ring C′ is 1,2,3-triazole. In certain embodiments, Ring C′ is 1,2,4-triazole. In certain embodiments, Ring C′ is pyrazole. In certain embodiments, each R^{C′} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-i-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, S-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{C′} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, at least one R^{C′} is C₃₋₆ carbocyclyl. In certain embodiments, at least one R^{C′} is cyclopropyl.

In certain embodiments, c′ is 0. In certain embodiments, c′ is 1. In certain embodiments, c′ is 2. In certain embodiments, c′ is 3. In certain embodiments, c′ is 4, as valency permits. In certain embodiments, c′ is 5, as valency permits.

In certain embodiments, Ring D′ is C₆ aryl (i.e., phenyl) or 5- to 6-membered heteroaryl (e.g., heteroaryl comprising one 5- or 6-membered ring and 1-4 heteroatoms selected from N, O, and S). In certain embodiments, Ring D′ is C₆ aryl (i.e., phenyl). In certain embodiments, Ring C′ is 5-membered heteroaryl. In certain embodiments, Ring C′ is 6-membered heteroaryl.

In certain embodiments, Ring D′ is phenyl.

In certain embodiments,

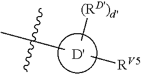

is

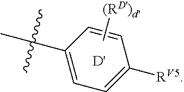

In certain embodiments, each R^{D′} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{D′} is independently halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl, or heterocyclyl is optionally substituted with one or more R^{u}.

In certain embodiments, d′ is 0. In certain embodiments, d′ is 1. In certain embodiments, d′ is 2. In certain embodiments, d′ is 3, as valency permit. In certain embodiments, d′ is 4, as valency permit. In certain embodiments, d′ is 5, as valency permit.

In certain embodiments, Ring E′ is C₃₋₈ carbocycle (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), or bicyclo[2.2.2]octanyl (C₈)) or 3- to 8-membered heterocycle (e.g., heterocycle comprising one or two 3- to 5-membered rings and 1 to 3 heteroatoms selected from N, O, and S).

In certain embodiments, Ring E′ is piperidinyl or tetrahydro-2H-pyranyl.

In certain embodiments, each R^{E′} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{E′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{E′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{E′} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl, or heterocyclyl is optionally substituted with one or more R^{u}.

In certain embodiments, e′ is 0. In certain embodiments, e′ is 1. In certain embodiments, e′ is 2. In certain embodiments, e′ is 3, as valency permits. In certain embodiments, e′ is 4, as valency permits. In certain embodiments, e′ is 5, as valency permits.

In certain embodiments, Ring F′ is C₆ aryl (i.e., phenyl) or 5- to 6-membered heteroaryl (e.g., heteroaryl comprising one or two 5- or 6-membered rings and 1-5 heteroatoms selected from N, O, and S).

In certain embodiments, Ring F′ is thiazolyl or 1,2,4-triazolyl, wherein the thiazolyl or 1,2,4-triazolyl is optionally substituted with one or more R^{u}.

In certain embodiments, each R^{F′} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{F′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{F′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{F′} is independently halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl, or heterocyclyl is optionally substituted with one or more R^{u}.

In certain embodiments, f′ is 0. In certain embodiments, f′ is 1. In certain embodiments, f is 2. In certain embodiments, f′ is 3, as valency permits. In certain embodiments, f′ is 4, as valency permits. In certain embodiments, f′ is 5, as valency permits.

In certain embodiments, R^{V1} is —N(R^{V1i})C(═O)R^{V1i} or 5- to 6-membered heteroaryl (e.g., heteroaryl comprising one 5- or 6-membered rings and 1-4 heteroatoms selected from N, O, and S) optionally substituted with one or more R^{u}. In certain embodiments, R^{V1} is —N(R^{V1i})C(═O)R^{V1ii}. In certain embodiments, R^{V1} is 5- to 6-membered heteroaryl (e.g., heteroaryl comprising one 5- or 6-membered rings and 1-4 heteroatoms selected from N, O, and S) optionally substituted with one or more R^{u}.

In certain embodiments, R^{V1i} is hydrogen, C₁₋₆ alkyl, C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, carbocyclyl, or heterocyclyl is optionally substituted with one or more R^{u}. In certain embodiments, R^{V1i} is hydrogen.

In certain embodiments, R^{V1ii} is C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{V1iia}.

In certain embodiments, R^{V1i} is C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{V1iia}

In certain embodiments, R^{V1i} is C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{V1iia}.

In certain embodiments, R^{V1i} is C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{V1iia}

In certain embodiments, R^{V1ii} is C₃₋₁₂ carbocyclyl optionally substituted with one or more R^{V1iia}. In certain embodiments, R^{V1ii} is C₃₋₆ carbocyclyl optionally substituted with one or more R^{V1iia}. In certain embodiments, R^{V1ii} is cyclopropyl optionally substituted with one or more R^{V1iia}

In certain embodiments, R^{V1ii} is unsubstituted. In certain embodiments, R^{V1ii} is optionally substituted with one R^{V1iia}. In certain embodiments, R^{V1ii} is optionally substituted with two R^{V1iia}. In certain embodiments, R^{V1i} is optionally substituted with three R^{V1iia}

In certain embodiments, each R^{V1iia} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), —(C₁₋₆ alkylene)-(3- to 12-membered heterocyclyl), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkylene, alkoxy, alkylamino, alkenyl, alkynyl, aryl, heteroaryl, carbocyclyl, heterocyclyl, aryl, or heteroaryl is optionally substituted with one or more R^{u}.

In certain embodiments, each R^{V1iia} is independently oxo, halogen, —CN, —NO₂, —OH, —NH₂, C₁₋₆ alkyl, —(C₁₋₆ alkylene)-(3- to 12-membered heterocyclyl), C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkylene, alkoxy, alkylamino, alkenyl, alkynyl, aryl, heteroaryl, carbocyclyl, heterocyclyl, aryl, or heteroaryl is optionally substituted with one or more R^{u}.

In certain embodiments, each R^{V1iia} is independently oxo, halogen, —CN, —NO₂, —OH, —NH₂, C₁₋₆ alkyl, —(C₁₋₆ alkylene)-(3- to 12-membered heterocyclyl), C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkylene, alkoxy, alkylamino, alkenyl, alkynyl, carbocyclyl, or heterocyclyl is optionally substituted with one or more R^{u}.

In certain embodiments, each R^{V1iia} is independently oxo, halogen, —CN, —NO₂, —OH, —NH₂, C₁₋₆ alkyl, —(C₁₋₆ alkylene)-(3- to 12-membered heterocyclyl), C₁₋₆ alkoxy, C₁₋₆ alkylamino, C₃₋₆ carbocyclyl, or 3- to 6-membered heterocyclyl, wherein the alkyl, alkylene, alkoxy, alkylamino, carbocyclyl, or heterocyclyl is optionally substituted with one or more R^{u}.

In certain embodiments, each R^{V1iia} is independently halogen, —CN, C₁₋₆ alkyl, —(C₁₋₆ alkylene)-(3- to 12-membered heterocyclyl), or C₁₋₆ alkylamino, wherein the alkyl, alkylene, alkylamino, or heterocyclyl is optionally substituted with one or more R^{u}. In certain embodiments, at least one R^{V1iia} is halogen. In certain embodiments, at least one R^{V1iia} is —CN. In certain embodiments, at least one R^{V1iia} is C₁₋₆ alkyl. In certain embodiments, at least one R^{V1iia} is —(C₁₋₆ alkylene)-(3- to 12-membered heterocyclyl). In certain embodiments, at least one R^{V1iia} is C₁₋₆ alkylamino.

In certain embodiments, R^{V2} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), or 3- to 12-membered heterocyclyl (e.g., heterocyclyl comprising one or two 3- to 8-membered rings and 1-5 heteroatoms selected from N, O, and S), wherein the alkyl, carbocyclyl, or heterocyclyl is optionally substituted with one or more R^{u}. In certain embodiments, R^{V2} is hydrogen. In certain embodiments, R^{V2} is C₁₋₆ alkyl optionally substituted with one or more R^{u}. In certain embodiments, R^{V2} is C₃₋₁₂ carbocyclyl optionally substituted with one or more R^{u}. In certain embodiments, R^{V2} is 3- to 12-membered heterocyclyl optionally substituted with one or more R^{u}.

In certain embodiments, R^{V2} is 2-propyl or t-butyl. In certain embodiments, R^{V2} is tetrahydro-2H-pyranyl, piperidinyl, or cyclohexyl.

In certain embodiments, R^{V3} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), 3- to 6-membered heterocyclyl (e.g., heterocyclyl comprising one 3- to 6-membered ring and 1-3 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u}.

In certain embodiments, R^{V3} is hydrogen or C₁₋₆ alkyl. In certain embodiments, R^{V3} is hydrogen. In certain embodiments, R^{V3} is C₁₋₆ alkyl.

In certain embodiments, each R^{V4} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), 3- to 6-membered heterocyclyl (e.g., heterocyclyl comprising one 3- to 6-membered ring and 1-3 heteroatoms selected from N, O, and S), wherein the alkyl, alkenyl, alkynyl, carbocyclyl, or heterocyclyl is optionally substituted with one or more R^{V4a}.

In certain embodiments, each R^{V4} is independently hydrogen or C₁₋₆ alkyl optionally substituted with one or more R^{V4a}. In certain embodiments, at least one R^{V4} is hydrogen. In certain embodiments, at least one R^{V4} is C₁₋₆ alkyl optionally substituted with one or more R^{V4a}. In certain embodiments, R^{V4} is methyl. In certain embodiments, one R^{V4} is hydrogen, and the other one R^{V4} is C₁₋₆ alkyl optionally substituted with one or more R^{V4a}. In certain embodiments, one R^{V4} is hydrogen, and the other R^{V4} is methyl optionally substituted with one or more R^{V4a}.

In certain embodiments, two R^{V4}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S), wherein the carbocycle or heterocycle is optionally substituted with one or more R^{u}.

In certain embodiments, R^{V4} is optionally substituted with one R^{V4a}. In certain embodiments, R^{V4} is optionally substituted with two R^{V4a}. In certain embodiments, R^{V4} is optionally substituted with three R^{V4a}.

In certain embodiments, each R^{V4a} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ heteroalkyl (e.g., heteroalkyl comprising 1 to 6 carbon atoms and 1 to 4 heteroatoms selected from N, O, and S), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, heteroalkyl, alkoxy, alkylamino, alkenyl, alkynyl, carbocyclyl, heterocyclyl, aryl, or heteroaryl is optionally substituted with one or more R^{u}.

In certain embodiments, each R^{V4a} is independently halogen, —CN, —NO₂, —OH, —NH₂, C₁₋₆ alkyl, C₁₋₆ heteroalkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 12-membered heteroaryl, wherein the alkyl, heteroalkyl, alkoxy, alkylamino, alkenyl, alkynyl, carbocyclyl, heterocyclyl, aryl, or heteroaryl is optionally substituted with one or more R^{u}.

In certain embodiments, each R^{V4a} is independently oxo, halogen, —CN, —NO₂, —OH, —NH₂, —C(═O)OR^{b}, —C(═O)NR^{c}R^{d}, C₁₋₆ alkyl, C₁₋₆ heteroalkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, heteroalkyl, alkoxy, alkylamino, alkenyl, alkynyl, carbocyclyl, heterocyclyl, aryl, or heteroaryl is optionally substituted with one or more R^{u}.

In certain embodiments, each R^{V4a} is independently oxo, halogen, —CN, —NO₂, —OH, —NH₂, —C(═O)OR, —C(═O)NR^{c}R^{d}, C₁₋₆ alkyl, C₁₋₆ heteroalkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, heteroalkyl, alkoxy, alkylamino, alkenyl, alkynyl, carbocyclyl, or heterocyclyl is optionally substituted with one or more R^{u}.

In certain embodiments, each R^{V4a} is independently oxo, halogen, —CN, —NO₂, —OH, —NH₂, —C(═O)OR^{b}, —C(═O)NR^{c}R^{d}, C₁₋₆ alkyl, C₁₋₆ heteroalkyl, C₁₋₆ alkoxy, C₁₋₆ alkylamino, C₃₋₆ carbocyclyl, or 3- to 6-membered heterocyclyl, wherein the alkyl, heteroalkyl, alkoxy, alkylamino, carbocyclyl, or heterocyclyl is optionally substituted with one or more R^{u}.

In certain embodiments, each R^{V4a} is independently —OH, —C(═O)OR^{b}, —C(═O) NR^{c}R^{d}, C₁₋₆ heteroalkyl, C₁₋₆ alkylamino, or 3- to 12-membered heterocyclyl, wherein the heteroalkyl, alkylamino, or heterocyclyl is optionally substituted with one or more R^{u}. In certain embodiments, each R^{V4a} is independently —OH, —C(═O)OR^{b}, —C(═O)NR^{c}R^{d}, C₁₋₆ heteroalkyl, C₁₋₆ alkylamino, or 3- to 12-membered heterocyclyl, wherein the heteroalkyl, alkylamino, or heterocyclyl is optionally substituted with one or more substituents selected from oxo, halogen, or —OH. In certain embodiments, at least one R^{V4a} is optionally substituted C₁₋₆ alkylamino. In certain embodiments, at least one R^{V4a} is optionally substituted 3- to 12-membered heterocyclyl.

In certain embodiments, R^{D′} and R^{V4}, together with the atoms to which they are bonded, form C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), or 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the carbocyclyl, heterocyclyl, aryl, or heteroaryl is optionally substituted with one or more R^{u}.

In certain embodiments, R^{D′} and R^{V4}, together with the intervening atoms, form C₃₋₁₂ carbocyclyl or 5- to 10-membered heteroaryl, wherein the carbocyclyl or heteroaryl is optionally substituted with one or more R^{u}.

In certain embodiments, R^{V5} is hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, 5- to 6-membered heteroaryl, or —C(═O)R^{a}, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, or —C(═O)R^{a}, wherein the alkyl, alkoxy, alkylamino, alkenyl, alkynyl, carbocyclyl, or heterocyclyl is optionally substituted with one or more R^{u}.

In certain embodiments, R^{V5} is hydrogen, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, or —C(═O)R^{a}, wherein the alkyl, alkoxy, alkylamino, carbocyclyl, or heterocyclyl is optionally substituted with one or more R^{u}.

In certain embodiments, R^{V5} is halogen, —C(═O)R^{a}, C₂₋₆ alkynyl, C₆₋₁₀ aryl, 5- to 6-membered heteroaryl, or —C(═O)R^{a}, wherein the alkynyl, aryl, or heteroaryl is optionally substituted with one or more R^{u}.

In certain embodiments, R^{V5} is halogen, C₂₋₆ alkynyl, or 5- to 6-membered heteroaryl optionally substituted with one or more R^{u}.

In certain embodiments, R^{V5} is —C(═O)R^{a}. In certain embodiments, R^{V5} is halogen. In certain embodiments, R^{V5} is C₂₋₆ alkynyl. In certain embodiments, R^{V5} is C₆₋₁₀ aryl. In certain embodiments, R^{V5} is 5-membered heteroaryl optionally substituted with one or more R^{u}. In certain embodiments, R^{V5} is 6-membered heteroaryl optionally substituted with one or more R^{u}.

In certain embodiments, R^{V5} is thiazolyl or 1,2,4-triazolyl, wherein the thiazolyl or 1,2,4-triazolyl is optionally substituted with one or more R^{u}.

In certain embodiments, L is of Formula II-2

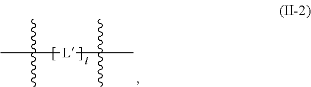

wherein:
each L′ is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O) N(R^{L′})—, —C(═O)O—, —N(R^{L′})—, —O—, —S—, or —S(═O)₂—, wherein the alkylene, heteroalkylene, alkenylene, alkynylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
l is an integer selected from 0 to 5.

In certain embodiments, each L′ is independently C₁₋₆ alkylene (e.g., methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), and hexylene (—CH₂CH₂CH₂CH₂CH₂CH₂—)), C₁₋₆ heteroalkylene (e.g., C₁₋₆ heteroalkylene comprising 1-7 heteroatoms selected from N, O, and S), C₂₋₆ alkenylene (e.g., ethenylene (C₂), 1-propenylene (C₃), 2-propenylene (C₃), 1-butenylene (C₄), 2-butenylene (C₄), butadienylene (C₄), pentenylene (C₅), pentadienylene (C₅), or hexenylene (C₆)), C₂₋₆ alkynylene (e.g., ethynylene (C₂), 1-propynylene (C₃), 2-propynylene (C₃), 1-butynylene (C₄), 2-butynylene (C₄), pentynylene (C₅), or hexynylene (C₆)), C₃₋₁₂ carbocyclylene (e.g., cyclopropylene (C₃), cyclopropenylene (C₃), cyclobutylene (C₄), cyclobutenylene (C₄), cyclopentylene (C₅), cyclopentenylene (C₅), cyclohexylene (C₆), cyclohexenylene (C₆), cyclohexadienylene (C₆), cycloheptylene (C₇), cycloheptenylene (C₇), cycloheptadienylene (C₇), cycloheptatrienylene (C₇), cyclooctylene (C₈), cyclooctenylene (C₈), bicyclo[2.2.1]heptanylene (C₇), bicyclo[2.2.2]octanylene (C₈), cyclononylene (C₉), cyclononenylene (C₉), cyclodecylene (C₁₀), cyclodecenylene (C₁₀), octahydro-1H-indenylene (C₉), decahydronaphthalenylene (C₁₀), or spiro[4.5]decanylene (C₁₀)), 3- to 12-membered heterocyclylene (e.g., heterocyclylene comprising one or two 3- to 8-membered rings and 1-5 heteroatoms selected from N, O, and S), C₆₋₁₀ arylene (e.g., phenylene or naphthylene), 5- to 10-membered heteroarylene (e.g., heteroarylene comprising one or two 5- or 6-membered rings and 1-5 heteroatoms selected from N, O, and S), —C(═O)—, —N(R^{L2})—, —O—, —S—, or —S(═O)₂—, wherein the alkylene, alkenylene, carbocyclylene, heterocyclylene, arylene, or heteroarylene is optionally substituted with one or more R^{u}.

In certain embodiments, each L′ is independently C₁₋₆ alkylene, C₁₋₆ heteroalkylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O) N(R^{L′})—, —C(═O)O—, —N(R^{L′})—, —O—, or —S(═O)₂—, wherein the alkylene, heteroalkylene, carbocyclylene, heterocyclylene, arylene, or heteroarylene is optionally substituted with one or more R^{u}.

In certain embodiments, each L′ is independently C₁₋₆ alkylene, C₁₋₆ heteroalkylene, C₂₋₆ alkynylene, C₃₋₁₂ carbocyclylene, 3- to 12-membered heterocyclylene, —C(═O)—, —C(═O) N(R^{L′})—, —C(═O)O—, —N(R^{L′})—, —O—, or —S(═O)₂—, wherein the alkylene, heteroalkylene, carbocyclylene, or heterocyclylene is optionally substituted with one or more R^{u}, and 1 is an integer selected from 1 to 4.

In certain embodiments, each occurrence of R^{L′} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, each occurrence of R^{L′} is independently hydrogen, C₁₋₆ alkyl, C₂₋₆ alkenyl, C₂₋₆ alkynyl, C₃₋₆ carbocyclyl, or 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, each occurrence of R^{L′} is independently hydrogen, C₁₋₆ alkyl, C₂₋₆ alkenyl, C₂₋₆ alkynyl, C₃₋₆ carbocyclyl, or 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O) NR^{c}R^{d}, wherein the alkyl, alkenyl, alkynyl, carbocyclyl, or heterocyclyl is optionally substituted with one or more R^{u}.

In certain embodiments, each occurrence of R^{L′} is independently hydrogen, C₁₋₆ alkyl, C₃₋₆ carbocyclyl, or 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, each occurrence of R^{L′} is hydrogen.

In certain embodiments, 1 is 0. In certain embodiments, 1 is 1. In certain embodiments, 1 is 2. In certain embodiments, 1 is 3. In certain embodiments, 1 is 4. In certain embodiments, 1 is 5. In certain embodiments, 1 is an integer selected from 1 to 4.

In certain embodiments, L is C₁₋₁₂ alkylene, C₃₋₁₂ carbocyclylene, 3- to 12-membered heterocyclylene, —C(═O)—, —N(R^{L′})—, or —O—, wherein the alkylene, carbocyclylene, or heterocyclylene is optionally substituted with one or more R^{u}.

In certain embodiments, L is —O—(C₁₋₆ alkylene)-, —(C₁₋₆ alkylene)-O—, —N(R^{L′})—(C₁₋₆ alkylene)-, —(C₁₋₆ alkylene)-N(R^{L′})—, —C(═O)—(C₁₋₆ alkylene)-, —(C₁₋₆ alkylene)-C(═O)—, —C(═O)-(3- to 12-membered heterocyclylene)-, -(3- to 12-membered heterocyclylene)-C(═O)—, -(3- to 12-membered heterocyclylene)-(C₁₋₆ alkylene)-, —(C₁₋₆ alkylene)-(3- to 12-membered heterocyclylene)-, —(C₁₋₆ alkylene)-(3- to 12-membered carbocyclylene)-, or -(3- to 12-membered carbocyclylene)-(C₁₋₆ alkylene)-, wherein the alkylene, carbocyclylene, or heterocyclylene is optionally substituted with one or more R^{u}.

In certain embodiments, L is absent, C₁₋₁₂ alkylene, C₁₋₆ heteroalkylene, C₃₋₁₂ carbocyclylene, *—C₁₋₁₂ alkylene-O—, *—C₁₋₁₂ alkynylene-O—, *—(C₁₋₁₂ alkylene)-(C₁₋₁₂ alkynylene)-, *—(C₁₋₁₂ alkylene)-O—(C₁₋₁₂ alkynylene)-, *—(C₃₋₁₂ carbocyclylene)-O—, *—(C₁₋₆ alkylene)-(C₃₋₁₂ carbocyclylene)-, *—(C₃₋₁₂ carbocyclylene)-(C₁₋₆ alkylene)-, *—(C₃₋₁₂ carbocyclylene)-(C₁₋₆ alkylene)-O—, *—(C₃₋₁₂ carbocyclylene)-(C₁₋₆ heteroalkylene)-, *—C(═O)—(C₁₋₆ alkylene)-, *—C(═O)—(C₃₋₁₂ carbocyclylene)-, *—C(═O)-(3- to 12-membered heterocyclylene)-, *—(C₁₋₆ alkylene)-(C₃₋₁₂ carbocyclylene)-O—, *-(3- to 12-membered heterocyclylene)-(C₁₋₆ alkylene)-(C₃₋₁₂ carbocyclylene)-, *—(C₁₋₆ alkylene)-(3- to 12-membered heterocyclylene)-, *—(C₁₋₆ alkylene)-(C₃₋₁₂ carbocyclylene)-C(═O) N(R^{L′})—, *-(3- to 12-membered heterocyclylene)-(C₁₋₆ alkylene)-, *—(C₁₋₆ alkylene)-C(═O)-(3- to 12-membered heterocyclylene)-, *—(C₁₋₆ alkylene)-(C₃₋₁₂ carbocyclylene)-C(═O)-(3- to 12-membered heterocyclylene)-, *—(C₃₋₁₂ carbocyclylene)-N(R^{L′})—C(═O)—(C₁₋₆ alkylene)-, *—(C₁₋₆ alkylene)-(C₃₋₁₂ carbocyclylene)-N(R^{L′})C(═O)—(C₁₋₆ alkylene)-, *-(3- to 12-membered heterocyclylene)-C(═O)—, *—(C₁₋₆ alkylene)-(3- to 12-membered heterocyclylene)-C(═O)—, *—(C₁₋₆ alkylene)-(C₃₋₁₂ carbocyclylene)-C(═O) N(R^{L′})—(C₁₋₆ alkylene)-, *—(C₁₋₆ alkylene)-(C₃₋₁₂ carbocyclylene)-C(═O)—(C₁₋₆ alkylene)-, *-(3- to 12-membered heterocyclylene)-C(═O)—(C₁₋₆ alkylene)-, *—(C₃₋₁₂ carbocyclylene)-N(R^{L′})C(═O)—(C₁₋₆ alkylene)-O—, *—(C₁₋₆ alkylene)-N(R^{L′})C(═O)—(C₁₋₆ alkylene)-, *—(C₁₋₆ alkylene)-O—(C₁₋₆ alkylene)-N(R^{L′})C(═O)—, *—(C₁₋₆ alkylene)-(C₃₋₁₂ carbocyclylene)-(C₁₋₆ alkylene)-, *—(C₁₋₆ alkylene)-(C₃₋₁₂ carbocyclylene)-O—, *—(C₁₋₆ alkylene)-(C₆₋₁₀ arylene)-, *—S(═O)₂—(C₃₋₁₂ carbocyclylene)-, or *—(C₁₋₆ alkylene)-(5- to 10-membered heteroarylene)-, wherein the alkylene, heteroalkylene, heterocyclylene, carbocyclylene, arylene, or heteroarylene is optionally substituted with one or more R^{u}, and *denotes attachment to T.

In certain embodiments, L is -(3- to 12-membered heterocyclylene)-(C₁₋₆ alkylene)-(3- to 12-membered heterocyclylene)-, -(3- to 12-membered carbocyclylene)-(C₁₋₆ alkylene)-(3- to 12-membered carbocyclylene)-, -(3- to 12-membered heterocyclylene)-(C₁₋₆ alkylene)-(3- to 12-membered carbocyclylene)-, -(3- to 12-membered carbocyclylene)-(C₁₋₆ alkylene)-(3- to 12-membered heterocyclylene)-, —(C₁₋₆ alkylene)-(3- to 12-membered heterocyclylene)-(C₁₋₆ alkylene)-, —(C₁₋₆ alkylene)-(3- to 12-membered carbocyclylene)-(C₁₋₆ alkylene)-, —(C₁₋₆ alkylene)-(3- to 12-membered heterocyclylene)-(C(═O))—, —(C(═O))-(3- to 12-membered heterocyclylene)-(C₁₋₆ alkylene)-, —(C₁₋₆ alkylene)-(3- to 12-membered carbocyclylene)-(C(═O))—, —(C(═O))-(3- to 12-membered carbocyclylene)-(C₁₋₆ alkylene)-, -(3- to 12-membered heterocyclylene)-(C₁₋₆ alkylene)-(C(═O))—, —(C(═O))—(C₁₋₆ alkylene)-(3- to 12-membered heterocyclylene)-, -(3- to 12-membered carbocyclylene)-(C₁₋₆ alkylene)-(C(═O))—, —(C(═O))—(C₁₋₆ alkylene)-(3- to 12-membered carbocyclylene)-, —(C₁₋₆ alkylene)-(C(═O))-(3- to 12-membered heterocyclylene)-, or -(3- to 12-membered heterocyclylene)-(C(═O))—(C₁₋₆ alkylene)-, —(C₁₋₆ alkylene)-(C(═O))-(3- to 12-membered carbocyclylene)-, -(3- to 12-membered carbocyclylene)-(C(═O))—(C₁₋₆ alkylene)-, -(3- to 12-membered heterocyclylene)-(C₁₋₆ alkylene)-(3- to 12-membered carbocyclylene), -(3- to 12-membered heterocyclylene)-C(═O)-(3- to 12-membered carbocyclylene), -(3- to 12-membered heterocyclylene)-O-(3- to 12-membered carbocyclylene), -(3- to 12-membered heterocyclylene)-N(R^{L′})-(3- to 12-membered carbocyclylene), -(3- to 12-membered carbocyclylene)-(C₁₋₆ alkylene)-(3- to 12-membered heterocyclylene), -(3- to 12-membered carbocyclylene)-C(═O)-(3- to 12-membered heterocyclylene), -(3- to 12-membered carbocyclylene)-O-(3- to 12-membered heterocyclylene), or -(3- to 12-membered carbocyclylene)-N(R^{L′})-(3- to 12-membered heterocyclylene), wherein the alkylene, carbocyclylene, or heterocyclylene is optionally substituted with one or more R^{u}.

In certain embodiments, L is absent, C₁₋₁₂ alkylene, C₁₋₆ heteroalkylene, C₃₋₁₂ carbocyclylene, *—C₁₋₁₂ alkylene-O—, *—(C₃₋₁₂ carbocyclylene)-O—, *—(C₁₋₆ alkylene)-(C₃₋₁₂ carbocyclylene)-, *—(C₃₋₁₂ carbocyclylene)-(C₁₋₆ alkylene)-, *—(C₃₋₁₂ carbocyclylene)-(C₁₋₆ alkylene)-O—, *—(C₃₋₁₂ carbocyclylene)-(C₁₋₆ heteroalkylene)-, *—C(═O)—(C₃₋₁₂ carbocyclylene)-, *—C(═O)-(3- to 12-membered heterocyclylene)-, *—(C₁₋₆ alkylene)-(C₃₋₁₂ carbocyclylene)-O—, *-(3- to 12-membered heterocyclylene)-(C₁₋₆ alkylene)-(C₃₋₁₂ carbocyclylene)-, *—(C₁₋₆ alkylene)-(3- to 12-membered heterocyclylene)-, *—(C₁₋₆ alkylene)-(C₃₋₁₂ carbocyclylene)-C(═O) N(R^{L′})—, *-(3- to 12-membered heterocyclylene)-(C₁₋₆ alkylene)-, *—(C₁₋₆ alkylene)-C(═O)-(3- to 12-membered heterocyclylene)-, *—(C₁₋₆ alkylene)-(C₃₋₁₂ carbocyclylene)-C(═O)-(3- to 12-membered heterocyclylene)-, *—(C₁₋₆ alkylene)-(C₃₋₁₂ carbocyclylene)-N(R^{L′})C(═O)—(C₁₋₆ alkylene)-, *—(C₃₋₁₂ carbocyclylene)-N(R^{L′})C(═O)—(C₁₋₆ alkylene)-O—, *—(C₁₋₆ alkylene)-N(R^{L′})C(═O)—(C₁₋₆ alkylene)-, *—(C₁₋₆ alkylene)-(C₃₋₁₂ carbocyclylene)-(C₁₋₆ alkylene)-, *—(C₁₋₆ alkylene)-(C₃₋₁₂ carbocyclylene)-O—, *—(C₁₋₆ alkylene)-(C₆₋₁₀ aryl)-, or *—(C₁₋₆ alkylene)-(5- to 10-membered heteroarylene)-, wherein the alkylene, heteroalkylene, heterocyclylene, carbocyclylene, arylene, or heteroarylene is optionally substituted with one or more R^{u}, and *denotes attachment to T.

In certain embodiments, L is of Formula I-2

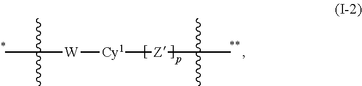

* denotes attachment to T and ** denotes attachment to V;
W is absent; or
W is C₁₋₃ alkylene, —O—, —NR^{W}—, or —(C═O)—, wherein the alkylene is optionally substituted by one or more R^{u};
Cy¹ is absent; or
Cy¹ is C₃₋₁₂ membered carbocyclylene or 3- to 12-membered heterocyclylene, wherein the carbocyclylene or heterocyclylene is optionally substituted by one or more R^{u};
Z is absent; or
Z is C₁₋₃ alkylene, —O—, —NR^{W}—, —(C═O)—, C₃₋₁₂ membered carbocyclylene, or 3- to 12-membered heterocyclylene, wherein the alkylene, carbocyclylene, or heterocyclylene is optionally substituted by one or more R^{u};
R^{W} is hydrogen or C₁₋₆ alkyl optionally substituted by one or more R^{u}; and p is an integer selected from 0 to 8;

In certain embodiments, W is C₁₋₃ alkylene, wherein the alkylene is optionally substituted by one or more R^{u}. In certain embodiments, W is —O—. In certain embodiments, W is —NR—. In certain embodiments, W is —(C═O)—.

In certain embodiments, R^{W} is hydrogen. In certain embodiments, R^{W} is C₁₋₆ alkyl optionally substituted by one or more R^{u}.

In certain embodiments, W is absent or —CH₂—.

In certain embodiments, Cy¹ is absent.

In certain embodiments, Cy¹ is C₃ carbocyclylene. In certain embodiments, Cy¹ is C₄ carbocyclylene. In certain embodiments, Cy¹ is C₅ carbocyclylene. In certain embodiments, Cy¹ is C₆ carbocyclylene. In certain embodiments, Cy¹ is C₇ carbocyclylene. In certain embodiments, Cy¹ is C₈ carbocyclylene. In certain embodiments, Cy¹ is C₉ carbocyclylene. In certain embodiments, Cy¹ is C₁₀ carbocyclylene. In certain embodiments, Cy¹ is C₁₁ carbocyclylene. In certain embodiments, Cy¹ is C₁₂ carbocyclylene.

In certain embodiments, Cy¹ is C₃₋₁₂ carbocyclylene. In certain embodiments, Cy¹ is C₃₋₁₁ carbocyclylene. In certain embodiments, Cy¹ is C₃₋₁₀ carbocyclylene. In certain embodiments, Cy¹ is C₃₋₉ carbocyclylene. In certain embodiments, Cy¹ is C₃₋₈ carbocyclylene. In certain embodiments, Cy¹ is C₃₋₇ carbocyclylene. In certain embodiments, Cy¹ is C₃₋₆ carbocyclylene. In certain embodiments, Cy¹ is C₃₋₅ carbocyclylene. In certain embodiments, Cy¹ is C₃₋₄ carbocyclylene. In certain embodiments, Cy¹ is C₄₋₁₂ carbocyclylene. In certain embodiments, Cy¹ is C₄₋₁₁ carbocyclylene. In certain embodiments, Cy¹ is C₄₋₁₀ carbocyclylene. In certain embodiments, Cy¹ is C₄₋₉ carbocyclylene. In certain embodiments, Cy¹ is C₄₋₈ carbocyclylene. In certain embodiments, Cy¹ is C₄₇ carbocyclylene. In certain embodiments, Cy¹ is C₄₋₆ carbocyclylene. In certain embodiments, Cy¹ is C₄₋₅ carbocyclylene. In certain embodiments, Cy¹ is C₅₋₁₂ carbocyclylene. In certain embodiments, Cy¹ is C₅₋₁₁ carbocyclylene. In certain embodiments, Cy¹ is C₅₋₁₀ carbocyclylene. In certain embodiments, Cy¹ is C₅₋₉ carbocyclylene. In certain embodiments, Cy¹ is C₅₋₈ carbocyclylene. In certain embodiments, Cy¹ is C₅₋₇carbocyclylene. In certain embodiments, Cy¹ is C₅₋₆ carbocyclylene. In certain embodiments, Cy¹ is C₆₋₁₂ carbocyclylene. In certain embodiments, Cy¹ is C₆₋₁₁ carbocyclylene. In certain embodiments, Cy¹ is C₆₋₁₀ carbocyclylene. In certain embodiments, Cy¹ is C₆₋₉ carbocyclylene. In certain embodiments, Cy¹ is C₆₋₈ carbocyclylene. In certain embodiments, Cy¹ is C₆₋₇ carbocyclylene. In certain embodiments, Cy¹ is C₇₋₁₂ carbocyclylene. In certain embodiments, Cy¹ is C₇₋₁₁ carbocyclylene. In certain embodiments, Cy¹ is C₇₋₁₀ carbocyclylene. In certain embodiments, Cy¹ is C₇₋₉ carbocyclylene. In certain embodiments, Cy¹ is C₇₋₈ carbocyclylene. In certain embodiments, Cy¹ is C₈₋₁₂ carbocyclylene. In certain embodiments, Cy¹ is C₈₋₁₁ carbocyclylene. In certain embodiments, Cy¹ is C₈₋₁₀ carbocyclylene. In certain embodiments, Cy¹ is C₈₋₉ carbocyclylene. In certain embodiments, Cy¹ is C₉₋₁₂ carbocyclylene. In certain embodiments, Cy¹ is C₉₋₁₁ carbocyclylene. In certain embodiments, Cy¹ is C₉₋₁₀ carbocyclylene. In certain embodiments, Cy¹ is C₁₀₋₁₂ carbocyclylene. In certain embodiments, Cy¹ is C₁₀₋₁₁ carbocyclylene. In certain embodiments, Cy¹ is C₁₁₋₁₂ carbocyclylene.

In certain embodiments, Cy¹ is 3-membered heterocyclylene. In certain embodiments, Cy¹ is 4-membered heterocyclylene. In certain embodiments, Cy¹ is 5-membered heterocyclylene. In certain embodiments, Cy¹ is 6-membered heterocyclylene. In certain embodiments, Cy¹ is 7-membered heterocyclylene. In certain embodiments, Cy¹ is 8-membered heterocyclylene. In certain embodiments, Cy¹ is 9-membered heterocyclylene. In certain embodiments, Cy¹ is 10-membered heterocyclylene. In certain embodiments, Cy¹ is 11-membered heterocyclylene. In certain embodiments, Ring D is 12-membered heterocyclylene.

In certain embodiments, Cy¹ is 3- to 12-membered heterocyclylene. In certain embodiments, Cy¹ is 3- to 11-membered heterocyclylene. In certain embodiments, Cy¹ is 3- to 10-membered heterocyclylene. In certain embodiments, Cy¹ is 3- to 9-membered heterocyclylene. In certain embodiments, Cy¹ is 3- to 8-membered heterocyclylene. In certain embodiments, Cy¹ is 3- to 7-membered heterocyclylene. In certain embodiments, Cy¹ is 3- to 6-membered heterocyclylene. In certain embodiments, Cy¹ is 3- to 5-membered heterocyclylene. In certain embodiments, Cy¹ is 3- to 4-membered heterocyclylene. In certain embodiments, Cy¹ is 4- to 12-membered heterocyclylene. In certain embodiments, Cy¹ is 4- to 11-membered heterocyclylene. In certain embodiments, Cy¹ is 4- to 10-membered heterocyclylene. In certain embodiments, Cy¹ is 4- to 9-membered heterocyclylene. In certain embodiments, Cy¹ is 4- to 8-membered heterocyclylene. In certain embodiments, Cy¹ is 4- to 7-membered heterocyclylene. In certain embodiments, Cy¹ is 4- to 6-membered heterocyclylene. In certain embodiments, Cy¹ is 4- to 5-membered heterocyclylene. In certain embodiments, Cy¹ is 5- to 12-membered heterocyclylene. In certain embodiments, Cy¹ is 5- to 11-membered heterocyclylene. In certain embodiments, Cy¹ is 5- to 10-membered heterocyclylene. In certain embodiments, Cy¹ is 5- to 9-membered heterocyclylene. In certain embodiments, Cy¹ is 5- to 8-membered heterocyclylene. In certain embodiments, Cy¹ is 5- to 7-membered heterocyclylene. In certain embodiments, Cy¹ is 5- to 6-membered heterocyclylene. In certain embodiments, Cy¹ is 6- to 12-membered heterocyclylene. In certain embodiments, Cy¹ is 6- to 11-membered heterocyclylene. In certain embodiments, Cy¹ is 6- to 10-membered heterocyclylene. In certain embodiments, Cy¹ is 6- to 9-membered heterocyclylene. In certain embodiments, Cy¹ is 6- to 8-membered heterocyclylene. In certain embodiments, Cy¹ is 6- to 7-membered heterocyclylene. In certain embodiments, Cy¹ is 8- to 12-membered heterocyclylene. In certain embodiments, Cy¹ is 8- to 11-membered heterocyclylene. In certain embodiments, Cy¹ is 8- to 10-membered heterocyclylene. In certain embodiments, Cy¹ is 8- to 9-membered heterocyclylene. In certain embodiments, Cy¹ is 9- to 12-membered heterocyclylene. In certain embodiments, Cy¹ is 9- to 11-membered heterocyclylene. In certain embodiments, Cy¹ is 9- to 10-membered heterocyclylene. In certain embodiments, Cy¹ is 10- to 12-membered heterocyclylene. In certain embodiments, Cy¹ is 10- to 11-membered heterocyclylene. In certain embodiments, Cy¹ is 11- to 12-membered heterocyclylene.

In certain embodiments, Cy¹ is heterocyclylene comprising 1 heteroatom selected from nitrogen, oxygen, and sulfur. In certain embodiments, Cy¹ is heterocyclylene comprising 2 heteroatoms selected from nitrogen, oxygen, and sulfur. In certain embodiments, Cy¹ is heterocyclylene comprising 3 heteroatoms selected from nitrogen, oxygen, and sulfur. In certain embodiments, Cy¹ is heterocyclylene comprising 4 heteroatoms selected from nitrogen, oxygen, and sulfur.

In certain embodiments, Cy¹ is heterocyclylene comprising 1 to 4 heteroatoms selected from nitrogen, oxygen, and sulfur. In certain embodiments, Cy¹ is heterocyclylene comprising 1 to 3 heteroatoms selected from nitrogen, oxygen, and sulfur. In certain embodiments, Cy¹ is heterocyclylene comprising 1 to 2 heteroatoms selected from nitrogen, oxygen, and sulfur. In certain embodiments, Cy¹ is heterocyclylene comprising 2 to 4 heteroatoms selected from nitrogen, oxygen, and sulfur. In certain embodiments, Cy¹ is heterocyclylene comprising 2 to 3 heteroatoms selected from nitrogen, oxygen, and sulfur. In certain embodiments, Cy¹ is heterocyclylene comprising 3 to 4 heteroatoms selected from nitrogen, oxygen, and sulfur.

In certain embodiments, Cy¹ is 4- to 11-membered carbocyclylene or 4- to 11-membered heterocyclylene, wherein the carbocyclylene or heterocyclylene is optionally substituted by one or more R^{u}.

In certain embodiments, Cy¹ is 4- to 11-membered carbocyclylene or 4- to 11-membered heterocyclylene selected from piperidinylene, cyclohexylene, azetidinylene optionally substituted by one or more R^{u}.

In certain embodiments, Z is absent.

In certain embodiments, Z is methylene optionally substituted by one or more R^{u}. In certain embodiments, Z is ethylene optionally substituted by one or more R^{u}. In certain embodiments, Z is propylene optionally substituted by one or more R^{u}.

In certain embodiments, Z is 3-membered heterocyclylene. In certain embodiments, Z is 4-membered heterocyclylene. In certain embodiments, Z is 5-membered heterocyclylene. In certain embodiments, Z is 6-membered heterocyclylene. In certain embodiments, Z is 7-membered heterocyclylene. In certain embodiments, Z is 8-membered heterocyclylene. In certain embodiments, Z is 9-membered heterocyclylene. In certain embodiments, Z is 10-membered heterocyclylene. In certain embodiments, Z is 11-membered heterocyclylene. In certain embodiments, Ring D is 12-membered heterocyclylene.

In certain embodiments, Z is 3- to 12-membered heterocyclylene. In certain embodiments, Z is 3- to 11-membered heterocyclylene. In certain embodiments, Z is 3- to 10-membered heterocyclylene. In certain embodiments, Z is 3- to 9-membered heterocyclylene. In certain embodiments, Z is 3- to 8-membered heterocyclylene. In certain embodiments, 7, is 3- to 7-membered heterocyclylene. In certain embodiments, Z is 3- to 6-membered heterocyclylene. In certain embodiments, Z is 3- to 5-membered heterocyclylene. In certain embodiments, Z is 3- to 4-membered heterocyclylene. In certain embodiments, Z is 4- to 12-membered heterocyclylene. In certain embodiments, Z is 4- to 11-membered heterocyclylene. In certain embodiments, Z is 4- to 10-membered heterocyclylene. In certain embodiments, Z is 4- to 9-membered heterocyclylene. In certain embodiments, Z is 4- to 8-membered heterocyclylene. In certain embodiments, Z is 4- to 7-membered heterocyclylene. In certain embodiments, Z is 4- to 6-membered heterocyclylene. In certain embodiments, Z is 4- to 5-membered heterocyclylene. In certain embodiments, Z is 5- to 12-membered heterocyclylene. In certain embodiments, Z is 5- to 11-membered heterocyclylene. In certain embodiments, Z is 5- to 10-membered heterocyclylene. In certain embodiments, Z is 5- to 9-membered heterocyclylene. In certain embodiments, Z is 5- to 8-membered heterocyclylene. In certain embodiments, Z is 5- to 7-membered heterocyclylene. In certain embodiments, Z is 5- to 6-membered heterocyclylene. In certain embodiments, Z is 6- to 12-membered heterocyclylene. In certain embodiments, Z is 6- to 11-membered heterocyclylene. In certain embodiments, Z is 6- to 10-membered heterocyclylene. In certain embodiments, Z is 6- to 9-membered heterocyclylene. In certain embodiments, Z is 6- to 8-membered heterocyclylene. In certain embodiments, Z is 6- to 7-membered heterocyclylene. In certain embodiments, Z is 8- to 12-membered heterocyclylene. In certain embodiments, Z is 8- to 11-membered heterocyclylene. In certain embodiments, Z is 8- to 10-membered heterocyclylene. In certain embodiments, Z is 8- to 9-membered heterocyclylene. In certain embodiments, Z is 9- to 12-membered heterocyclylene. In certain embodiments, Z is 9- to 11-membered heterocyclylene. In certain embodiments, Z is 9- to 10-membered heterocyclylene. In certain embodiments, Z is 10- to 12-membered heterocyclylene. In certain embodiments, Z is 10- to 11-membered heterocyclylene. In certain embodiments, Z is 11- to 12-membered heterocyclylene.

In certain embodiments, Z is heterocyclylene comprising 1 heteroatom selected from nitrogen, oxygen, and sulfur. In certain embodiments, Z is heterocyclylene comprising 2 heteroatoms selected from nitrogen, oxygen, and sulfur. In certain embodiments, Z is heterocyclylene comprising 3 heteroatoms selected from nitrogen, oxygen, and sulfur. In certain embodiments, Z is heterocyclylene comprising 4 heteroatoms selected from nitrogen, oxygen, and sulfur.

In certain embodiments, Z is heterocyclylene comprising 1 to 4 heteroatoms selected from nitrogen, oxygen, and sulfur. In certain embodiments, Z is heterocyclylene comprising 1 to 3 heteroatoms selected from nitrogen, oxygen, and sulfur. In certain embodiments, Z is heterocyclylene comprising 1 to 2 heteroatoms selected from nitrogen, oxygen, and sulfur. In certain embodiments, Z is heterocyclylene comprising 2 to 4 heteroatoms selected from nitrogen, oxygen, and sulfur. In certain embodiments, Z is heterocyclylene comprising 2 to 3 heteroatoms selected from nitrogen, oxygen, and sulfur. In certain embodiments, Z is heterocyclylene comprising 3 to 4 heteroatoms selected from nitrogen, oxygen, and sulfur.

In certain embodiments, Z is —C(═O)—, C₁₋₆ alkylene, —O—(C₁₋₆ alkylene)-, —(C₁₋₆ alkylene)-(C(═O))—O—, —(C₁₋₆ alkylene)-O—, —C(═O)—(C₁₋₆ alkylene)-, —(C₁₋₆ alkylene)-C(═O)—, 3- to 12-membered heterocyclylene, —C(═O)-(3- to 12-membered heterocyclylene)-, -(3- to 12-membered heterocyclylene)-C(═O)—, -(3- to 12-membered heterocyclylene)-(C₁₋₆ alkylene)-, —(C₁₋₆ alkylene)-(3- to 12-membered heterocyclylene)-, —(C₁₋₆ alkylene)-(3- to 12-membered heterocyclylene)-(C₁₋₆ alkylene)-, —(C₁₋₆ alkylene)-(3- to 12-membered heterocyclylene)-(C(═O))—, —(C(═O))-(3- to 12-membered heterocyclylene)-(C₁₋₆ alkylene)-, -(3- to 12-membered heterocyclylene)-(C₁₋₆ alkylene)-(C(═O))—, —(C(═O))—(C₁₋₆ alkylene)-(3- to 12-membered heterocyclylene)-, —(C₁₋₆ alkylene)-(C(═O))-(3- to 12-membered heterocyclylene)-, or -(3- to 12-membered heterocyclylene)-(C(═O))—(C₁₋₆ alkylene)-, wherein the alkylene or heterocyclylene is optionally substituted by one or more R^{u}.

In certain embodiments, Z is C₁₋₆ alkylene or *—(C₁₋₆ alkylene)-(3- to 12-membered carbocyclylene)-, wherein the alkylene or carbocyclylene is optionally substituted by one or more R^{u}, and *denotes attachment to T.

In certain embodiments, each R^{a} is independently C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{a} is independently C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{a} is independently C₁₋₆ alkyl, C₂₋₆ alkenyl, C₂₋₆ alkynyl, C₃₋₆ carbocyclyl, or 3- to 6-membered heterocyclyl.

In certain embodiments, each R^{a} is independently C₁₋₆ alkyl, C₃₋₆ carbocyclyl, or 3- to 6-membered heterocyclyl, wherein the alkyl, carbocyclyl, or heterocyclyl is optionally substituted with one or more R^{u}.

In certain embodiments, each R^{b} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{b} is independently hydrogen, C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{b} is independently hydrogen, C₁₋₆ alkyl, C₂₋₆ alkenyl, C₂₋₆ alkynyl, C₃₋₆ carbocyclyl, or 3- to 6-membered heterocyclyl.

In certain embodiments, each R^{b} is independently hydrogen, C₁₋₆ alkyl, C₃₋₆ carbocyclyl, or 3- to 6-membered heterocyclyl, or C₂₋₆ alkynyl, wherein the alkyl, carbocyclyl, or heterocyclyl is optionally substituted with one or more R^{u}.

In certain embodiments, each Re and each R^{d} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each Re and each R^{d} is independently hydrogen, C₁₋₆ alkyl, C₃₋₆ carbocyclyl, or 3- to 6-membered heterocyclyl, wherein the alkyl, carbocyclyl, or heterocyclyl is optionally substituted with one or more R^{u}.

In certain embodiments, R^{c} and R^{d}, together with the nitrogen atom to which they are attached, form 3- to 12-membered heterocyclyl (e.g., heterocyclyl comprising one or two 3- to 8-membered rings and 1-5 heteroatoms selected from N, O, and S), wherein the heterocyclyl is optionally substituted with one or more R^{u}.

In certain embodiments, R^{a}, R^{b}, R^{c}, and R^{d} is independently and optionally substituted with one or more R^{z}.

In certain embodiments, R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, each R^{u} is independently oxo, halogen, —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, two R^{u}, together with the carbon atom(s) to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), 3- to 6-membered heterocyclyl (e.g., heterocyclyl comprising one 3- to 6-membered ring and 1-3 heteroatoms selected from N, O, and S), C₆ aryl (i.e., phenyl), or 5- to 6-membered heteroaryl (e.g., heteroaryl comprising one 5- or 6-membered ring and 1-3 heteroatoms selected from N, O, and S), wherein the carbocyclyl, heterocyclyl, aryl, or heteroaryl is optionally substituted with one or more Rz.

In certain embodiments, two R^{u}, together with the carbon atom(s) to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S), wherein the carbocyclyl or heterocyclyl is optionally substituted with one or more Rz.

In certain embodiments, two geminal R^{u}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S), wherein the carbocyclyl or heterocyclyl is optionally substituted with one or more R².

Embodiments of the variables in any of the Formulae described herein, e.g., Formulae I-2, II, II-1, II-2, II-3-i, II-3-ii, II-3-iii, II-3-iv, II-3-v, II-3-vi, II-1-i, II-1-ii, II-1-i-1, II-1-i-2, II-1-ii-1, II-1-ii-2, II-1-i-3, II-1-i-4, II-1-ii-3, or II-1-ii-4, as applicable, are described below. Any of the variables can be any moiety as described in the embodiments below. In addition, the combination of any moieties described for any of the variables, as applicable, with any moieties described for any of the remaining variables, are also contemplated.

When a range of values is listed, each discrete value and sub-range within the range are also contemplated. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

In certain embodiments, the compound is selected from the compounds in Tables 1 and 2, or a pharmaceutically acceptable salt thereof.

TABLE 1

| Com- | | |
|---|---|---|
| pound | | |
| No. | Structure | Chemical Name |
| A1 | 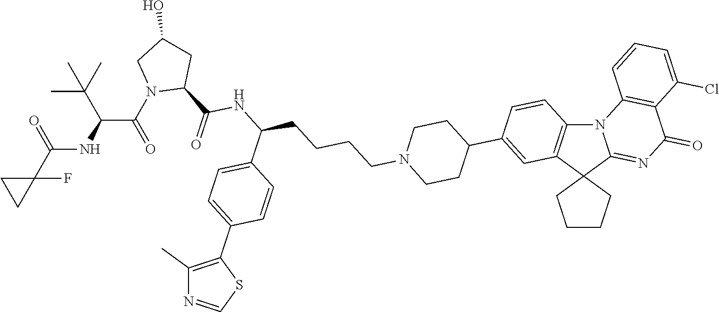 [SMILES: Cc1ncsc1-c1ccc([C@H](CCCCN2CCC(c3ccc4c(c3)C3(CCCC3)c3nc(=O)c5c(Cl)cccc5n3-4)CC2)NC(=O)[C@@H]2C[C@@H](O)CN2C(=O)[C@@H](NC(=O)C2(F)CC2)C(C)(C)C)cc1] | (2S,4R)-N-((S)-5-(4-(4′-chloro-5′-oxo- 5′H-spiro[cyclopentane-1,7′- indolo[1,2-a]quinazolin]-9′- yl)piperidin-1-yl)-1-(4-(4- methylthiazol-5-yl)phenyl)pentyl)-1- ((S)-2-(1-fluorocyclopropane-1- carboxamido)-3,3-dimethylbutanoyl)- 4-hydroxypyrrolidine-2-carboxamide |
| A2 | 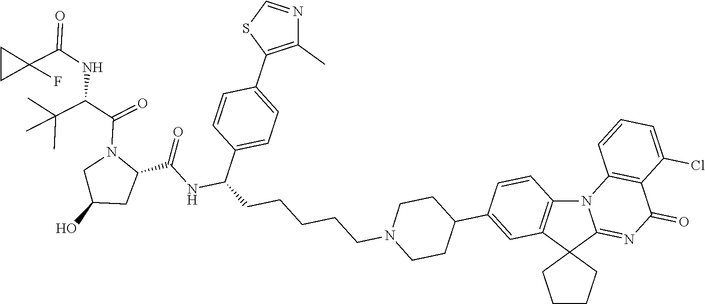 [SMILES: Cc1ncsc1-c1ccc([C@H](CCCCCN2CCC(c3ccc4c(c3)C3(CCCC3)c3nc(=O)c5c(Cl)cccc5n3-4)CC2)NC(=O)[C@@H]2C[C@@H](O)CN2C(=O)[C@@H](NC(=O)C2(F)CC2)C(C)(C)C)cc1] | (2S,4R)-N-((S)-6-(4-(4′-chloro-5′-oxo- 5′H-spiro[cyclopentane-1,7′- indolo[1,2-a]quinazolin]-9′- yl)piperidin-1-yl)-1-(4-(4- methylthiazol-5-yl)phenyl)hexyl)-1- ((S)-2-(1-fluorocyclopropane-1- carboxamido)-3,3-dimethylbutanoyl)- 4-hydroxypyrrolidine-2-carboxamide |
| A3 | 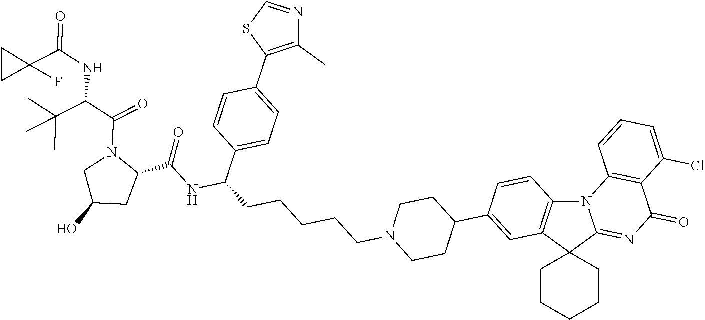 [SMILES: Cc1ncsc1-c1ccc([C@H](CCCCCN2CCC(c3ccc4c(c3)C3(CCCCC3)c3nc(=O)c5c(Cl)cccc5n3-4)CC2)NC(=O)[C@@H]2C[C@@H](O)CN2C(=O)[C@@H](NC(=O)C2(F)CC2)C(C)(C)C)cc1] | (2S,4R)-N-((S)-6-(4-(4′-chloro-5′-oxo- 5′H-spiro[cyclohexane-1,7′-indolo[1,2- a]quinazolin]-9′-yl)piperidin-1-yl)-1- (4-(4-methylthiazol-5- yl)phenyl)hexyl)-1-((S)-2-(1- fluorocyclopropane-1-carboxamido)- 3,3-dimethylbutanoyl)-4- hydroxypyrrolidine-2-carboxamide |
| A4 | 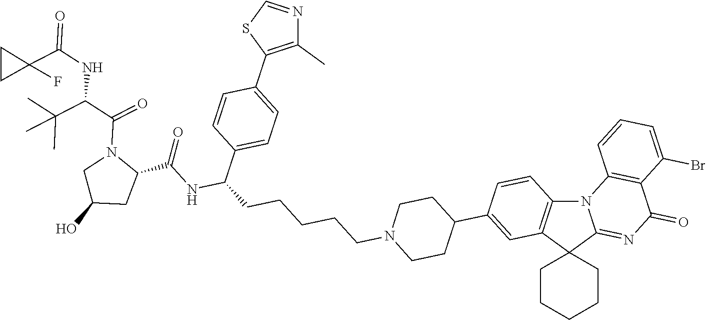 [SMILES: Cc1ncsc1-c1ccc([C@H](CCCCCN2CCC(c3ccc4c(c3)C3(CCCCC3)c3nc(=O)c5c(Br)cccc5n3-4)CC2)NC(=O)[C@@H]2C[C@@H](O)CN2C(=O)[C@@H](NC(=O)C2(F)CC2)C(C)(C)C)cc1] | (2S,4R)-N-((S)-6-(4-(4′-bromo-5′-oxo- 5′H-spiro[cyclohexane-1,7′-indolo[1,2- a]quinazolin]-9′-yl)piperidin-1-yl)-1- (4-(4-methylthiazol-5- yl)phenyl)hexyl)-1-((S)-2-(1- fluorocyclopropane-1-carboxamido)- 3,3-dimethylbutanoyl)-4- hydroxypyrrolidine-2-carboxamide |
| A5 | 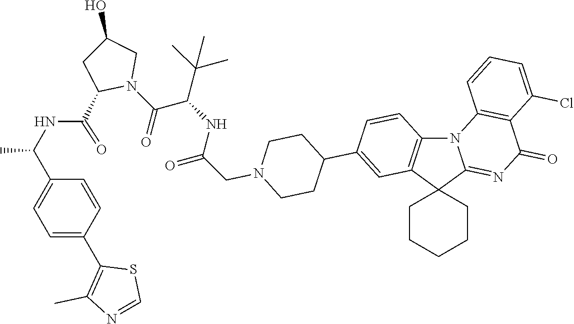 [SMILES: Cc1ncsc1-c1ccc([C@H](C)NC(=O)[C@@H]2C[C@@H](O)CN2C(=O)[C@@H](NC(=O)CN2CCC(c3ccc4c(c3)C3(CCCCC3)c3nc(=O)c5c(Cl)cccc5n3-4)CC2)C(C)(C)C)cc1] | (2S,4R)-1-((S)-2-(2-(4-(4′-chloro-5′- oxo-5′H-spiro[cyclohexane-1,7′- indolo[1,2-a]quinazolin]-9′- yl)piperidin-1-yl)acetamido)-3,3- dimethylbutanoyl)-4-hydroxy-N-((S)- 1-(4-(4-methylthiazol-5- yl)phenyl)ethyl)pyrrolidine-2- carboxamide |
| A6 | 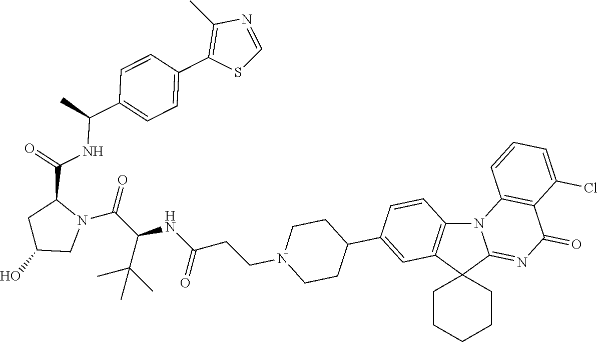 [SMILES: Cc1ncsc1-c1ccc([C@H](C)NC(=O)[C@@H]2C[C@@H](O)CN2C(=O)[C@@H](NC(=O)CCN2CCC(c3ccc4c(c3)C3(CCCCC3)c3nc(=O)c5c(Cl)cccc5n3-4)CC2)C(C)(C)C)cc1] | (2S,4R)-1-((S)-2-(3-(4-(4′-chloro-5′- oxo-5′H-spiro[cyclohexane-1,7′- indolo[1,2-a]quinazolin]-9′- yl)piperidin-1-yl)propanamido)-3,3- dimethylbutanoyl)-4-hydroxy-N-((S)- 1-(4-(4-methylthiazol-5- yl)phenyl)ethyl)pyrrolidine-2- carboxamide |
| A7 | 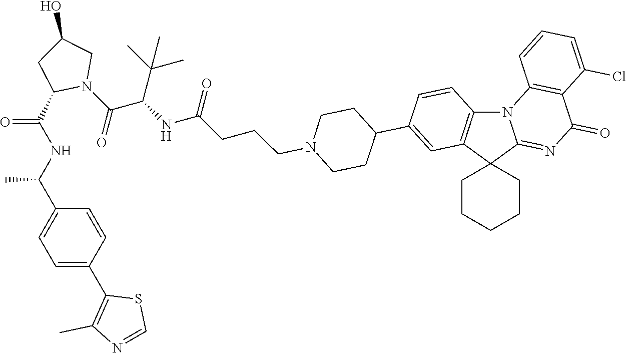 [SMILES: Cc1ncsc1-c1ccc([C@H](C)NC(=O)[C@@H]2C[C@@H](O)CN2C(=O)[C@@H](NC(=O)CCCN2CCC(c3ccc4c(c3)C3(CCCCC3)c3nc(=O)c5c(Cl)cccc5n3-4)CC2)C(C)(C)C)cc1] | (2S,4R)-1-((S)-2-(4-(4-(4′-chloro-5′- oxo-5′H-spiro[cyclohexane-1,7′- indolo[1,2-a]quinazolin]-9′- yl)piperidin-1-yl)butanamido)-3,3- dimethylbutanoyl)-4-hydroxy-N-((S)- 1-(4-(4-methylthiazol-5- yl)phenyl)ethyl)pyrrolidine-2- carboxamide |
| A8 | 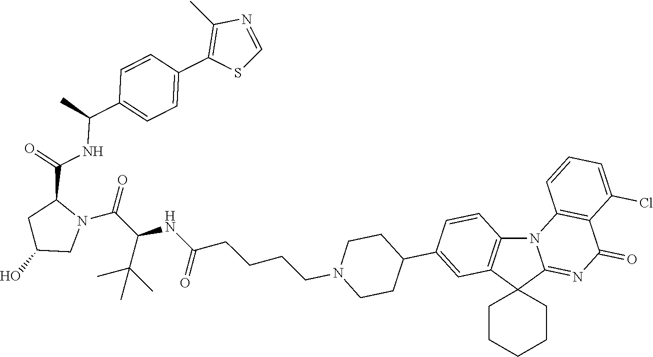 [SMILES: Cc1ncsc1-c1ccc([C@H](C)NC(=O)[C@@H]2C[C@@H](O)CN2C(=O)[C@@H](NC(=O)CCCCN2CCC(c3ccc4c(c3)C3(CCCCC3)c3nc(=O)c5c(Cl)cccc5n3-4)CC2)C(C)(C)C)cc1] | (2S,4R)-1-((S)-2-(5-(4-(4′-chloro-5′- oxo-5′H-spiro[cyclohexane-1,7′- indolo[1,2-a]quinazolin]-9′- yl)piperidin-1-yl)pentanamido)-3,3- dimethylbutanoyl)-4-hydroxy-N-((S)- 1-(4-(4-methylthiazol-5- yl)phenyl)ethyl)pyrrolidine-2- carboxamide |
| A9 | 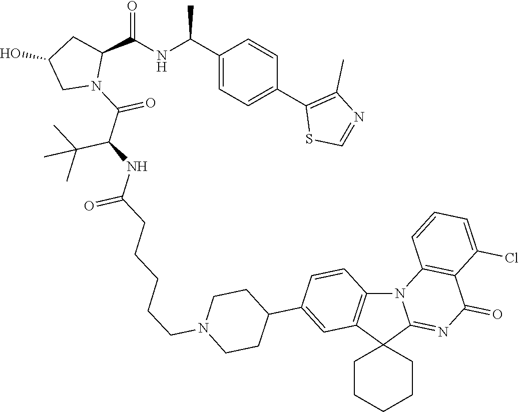 [SMILES: Cc1ncsc1-c1ccc([C@H](C)NC(=O)[C@@H]2C[C@@H](O)CN2C(=O)[C@@H](NC(=O)CCCCCN2CCC(c3ccc4c(c3)C3(CCCCC3)c3nc(=O)c5c(Cl)cccc5n3-4)CC2)C(C)(C)C)cc1] | (2S,4R)-1-((S)-2-(6-(4-(4′-chloro-5′- oxo-5′H-spiro[cyclohexane-1,7′- indolo[1,2-a]quinazolin]-9′- yl)piperidin-1-yl)hexanamido)-3,3- dimethylbutanoyl)-4-hydroxy-N-((S)- 1-(4-(4-methylthiazol-5- yl)phenyl)ethyl)pyrrolidine-2- carboxamide |
| A10 | 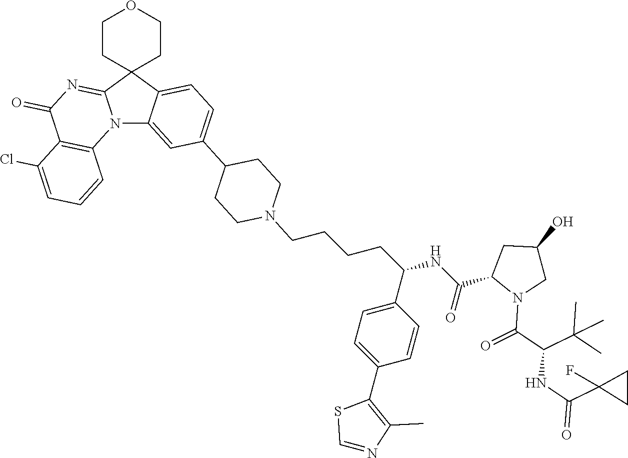 [SMILES: Cc1ncsc1-c1ccc([C@H](CCCCN2CCC(c3ccc4c(c3)-n3c(nc(=O)c5c(Cl)cccc53)C43CCOCC3)CC2)NC(=O)[C@@H]2C[C@@H](O)CN2C(=O)[C@@H](NC(=O)C2(F)CC2)C(C)(C)C)cc1] | (2S,4R)-N-((S)-5-(4-(4-chloro-5-oxo- 2′,3′,5′,6′-tetrahydro-5H- spiro[indolo[1,2-a]quinazoline-7,4′- pyran]-10-yl)piperidin-1-yl)-1-(4-(4- methylthiazol-5-yl)phenyl)pentyl)-1- ((S)-2-(1-fluorocyclopropane-1- carboxamido)-3,3-dimethylbutanoyl)- 4-hydroxypyrrolidine-2-carboxamide |
| A11 | 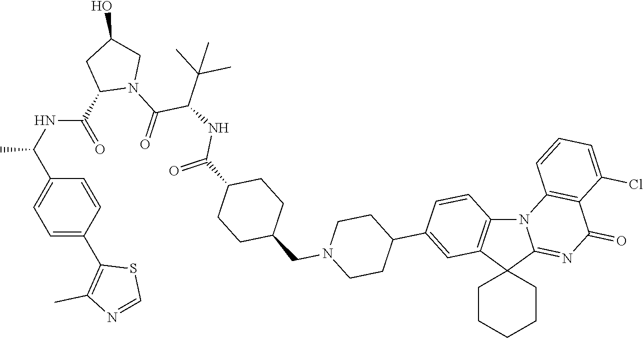 [SMILES: Cc1ncsc1-c1ccc([C@H](C)NC(=O)[C@@H]2C[C@@H](O)CN2C(=O)[C@@H](NC(=O)[C@H]2CC[C@H](CN3CCC(c4ccc5c(c4)C4(CCCCC4)c4nc(=O)c6c(Cl)cccc6n4-5)CC3)CC2)C(C)(C)C)cc1] | (2S,4R)-1-((S)-2-((1r,4S)-4-((4-(4′- chloro-5′-oxo-5′H-spiro[cyclohexane- 1,7′-indolo[1,2-a]quinazolin]-9′- yl)piperidin-1-yl)methyl)cyclohexane- 1-carboxamido)-3,3- dimethylbutanoyl)-4-hydroxy-N-((S)- 1-(4-(4-methylthiazol-5- yl)phenyl)ethyl)pyrrolidine-2- carboxamide |
| A12 | 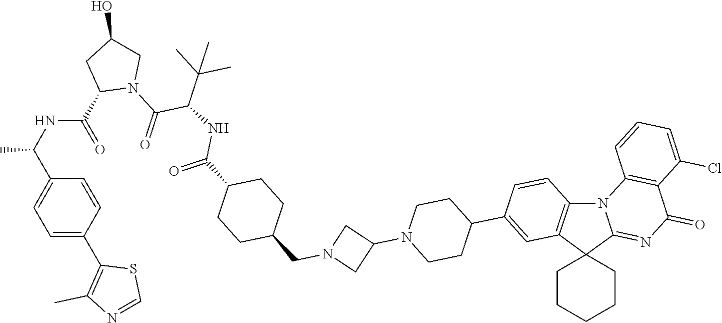 [SMILES: Cc1ncsc1-c1ccc([C@H](C)NC(=O)[C@@H]2C[C@@H](O)CN2C(=O)[C@@H](NC(=O)[C@H]2CC[C@H](CN3CC(N4CCC(c5ccc6c(c5)C5(CCCCC5)c5nc(=O)c7c(Cl)cccc7n5-6)CC4)C3)CC2)C(C)(C)C)cc1] | (2S,4R)-1-((S)-2-((1r,4S)-4-((3-(4-(4′- chloro-5′-oxo-5′H-spiro[cyclohexane- 1,7′-indolo[1,2-a]quinazolin]-9′- yl)piperidin-1-yl)azetidin-1- yl)methyl)cyclohexane-1- carboxamido)-3,3-dimethylbutanoyl)- 4-hydroxy-N-((S)-1-(4-(4- methylthiazol-5- yl)phenyl)ethyl)pyrrolidine-2- carboxamide |
| A13 | 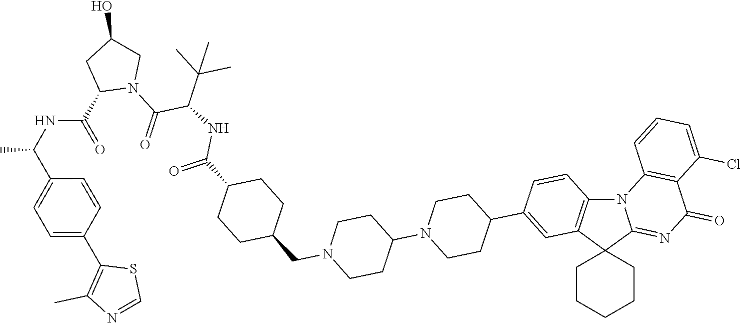 [SMILES: Cc1ncsc1-c1ccc([C@H](C)NC(=O)[C@@H]2C[C@@H](O)CN2C(=O)[C@@H](NC(=O)[C@H]2CC[C@H](CN3CCC(N4CCC(c5ccc6c(c5)C5(CCCCC5)c5nc(=O)c7c(Cl)cccc7n5-6)CC4)CC3)CC2)C(C)(C)C)cc1] | (2S,4R)-1-((S)-2-((1r,4S)-4-((4-(4′- chloro-5′-oxo-5′H-spiro[cyclohexane- 1,7′-indolo[1,2-a]quinazolin]-9′-yl)- [1,4′-bipiperidin]-1′- yl)methyl)cyclohexane-1- carboxamido)-3,3-dimethylbutanoyl)- 4-hydroxy-N-((S)-1-(4-(4- methylthiazol-5- yl)phenyl)ethyl)pyrrolidine-2- carboxamide |
| A14 | 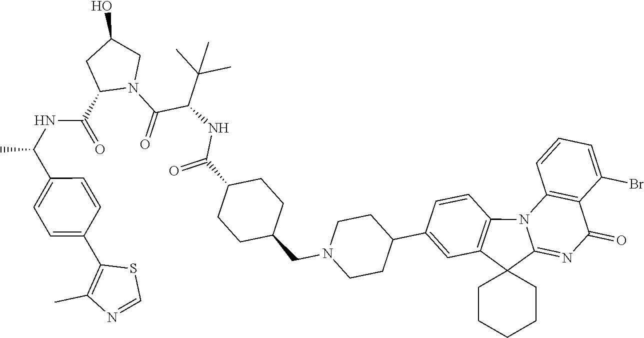 [SMILES: Cc1ncsc1-c1ccc([C@H](C)NC(=O)[C@@H]2C[C@@H](O)CN2C(=O)[C@@H](NC(=O)[C@H]2CC[C@H](CN3CCC(c4ccc5c(c4)C4(CCCCC4)c4nc(=O)c6c(Br)cccc6n4-5)CC3)CC2)C(C)(C)C)cc1] | (2S,4R)-1-((S)-2-((1r,4S)-4-((4-(4′- bromo-5′-oxo-5′H-spiro[cyclohexane- 1,7′-indolo[1,2-a]quinazolin]-9′- yl)piperidin-1-yl)methyl)cyclohexane- 1-carboxamido)-3,3- dimethylbutanoyl)-4-hydroxy-N-((S)- 1-(4-(4-methylthiazol-5- yl)phenyl)ethyl)pyrrolidine-2- carboxamide |
| A15 | 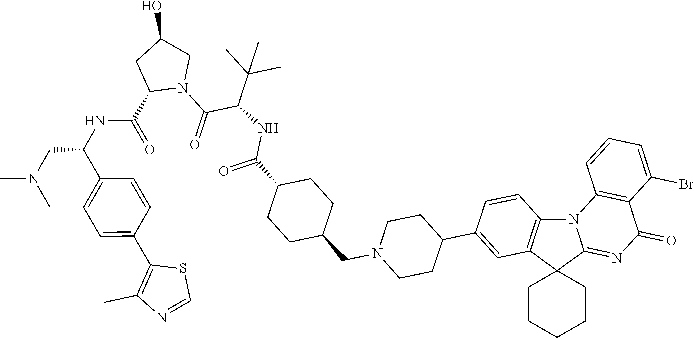 [SMILES: Cc1ncsc1-c1ccc([C@H](CN(C)C)NC(=O)[C@@H]2C[C@@H](O)CN2C(=O)[C@@H](NC(=O)[C@H]2CC[C@H](CN3CCC(c4ccc5c(c4)C4(CCCCC4)c4nc(=O)c6c(Br)cccc6n4-5)CC3)CC2)C(C)(C)C)cc1] | (2S,4R)-1-((S)-2-((1r,4S)-4-((4-(4′- bromo-5′-oxo-5′H-spiro[cyclohexane- 1,7′-indolo[1,2-a]quinazolin]-9′- yl)piperidin-1-yl)methyl)cyclohexane- 1-carboxamido)-3,3- dimethylbutanoyl)-N-((R)-2- (dimethylamino)-1-(4-(4- methylthiazol-5-yl)phenyl)ethyl)-4- hydroxypyrrolidine-2-carboxamide |
| A16 | 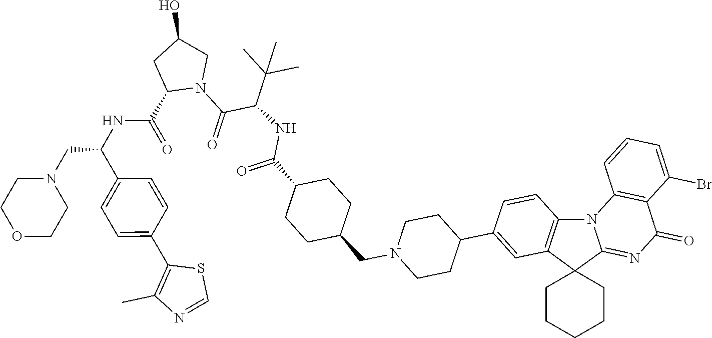 [SMILES: Cc1ncsc1-c1ccc([C@H](CN2CCOCC2)NC(=O)[C@@H]2C[C@@H](O)CN2C(=O)[C@@H](NC(=O)[C@H]2CC[C@H](CN3CCC(c4ccc5c(c4)C4(CCCCC4)c4nc(=O)c6c(Br)cccc6n4-5)CC3)CC2)C(C)(C)C)cc1] | (2S,4R)-1-((S)-2-((1r,4S)-4-((4-(4′- bromo-5′-oxo-5′H-spiro[cyclohexane- 1,7′-indolo[1,2-a]quinazolin]-9′- yl)piperidin-1-yl)methyl)cyclohexane- 1-carboxamido)-3,3- dimethylbutanoyl)-4-hydroxy-N-((R)- 1-(4-(4-methylthiazol-5-yl)phenyl)-2- morpholinoethyl)pyrrolidine-2- carboxamide |
| A17 | 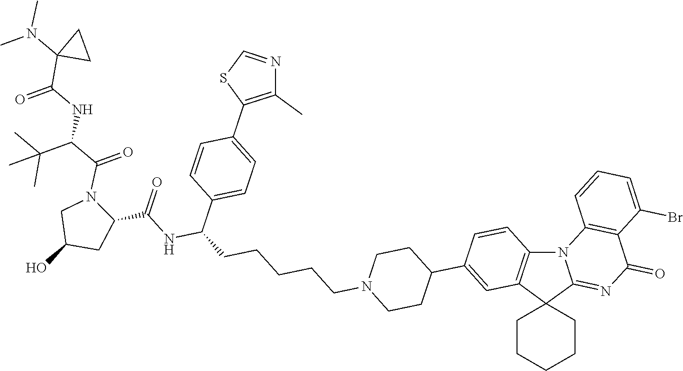 [SMILES: Cc1ncsc1-c1ccc([C@H](CCCCCN2CCC(c3ccc4c(c3)C3(CCCCC3)c3nc(=O)c5c(Br)cccc5n3-4)CC2)NC(=O)[C@@H]2C[C@@H](O)CN2C(=O)[C@@H](NC(=O)C2(N(C)C)CC2)C(C)(C)C)cc1] | (2S,4R)-N-((S)-6-(4-(4′-bromo-5′-oxo- 5′H-spiro[cyclohexane-1,7′-indolo[1,2- a]quinazolin]-9′-yl)piperidin-1-yl)-1- (4-(4-methylthiazol-5- yl)phenyl)hexyl)-1-((S)-2-(1- (dimethylamino)cyclopropane-1- carboxamido)-3,3-dimethylbutanoyl)- 4-hydroxypyrrolidine-2-carboxamide |
| A18 | 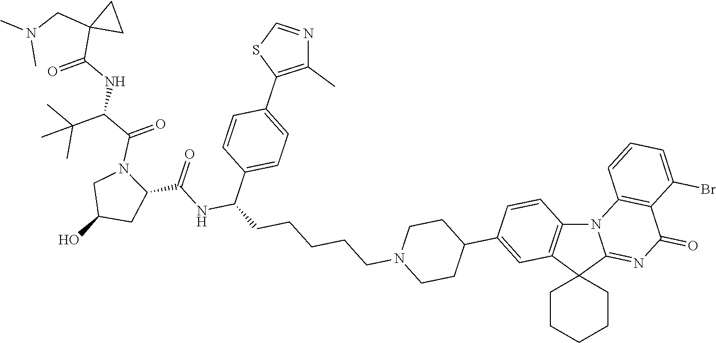 [SMILES: Cc1ncsc1-c1ccc([C@H](CCCCCN2CCC(c3ccc4c(c3)C3(CCCCC3)c3nc(=O)c5c(Br)cccc5n3-4)CC2)NC(=O)[C@@H]2C[C@@H](O)CN2C(=O)[C@@H](NC(=O)C2(CN(C)C)CC2)C(C)(C)C)cc1] | (2S,4R)-N-((S)-6-(4-(4′-bromo-5′-oxo- 5′H-spiro[cyclohexane-1,7′-indolo[1,2- a]quinazolin]-9′-yl)piperidin-1-yl)-1- (4-(4-methylthiazol-5- yl)phenyl)hexyl)-1-((S)-2-(1- ((dimethylamino)methyl)cyclopropane- 1-carboxamido)-3,3- dimethylbutanoyl)-4- hydroxypyrrolidine-2-carboxamide |
| A19 | 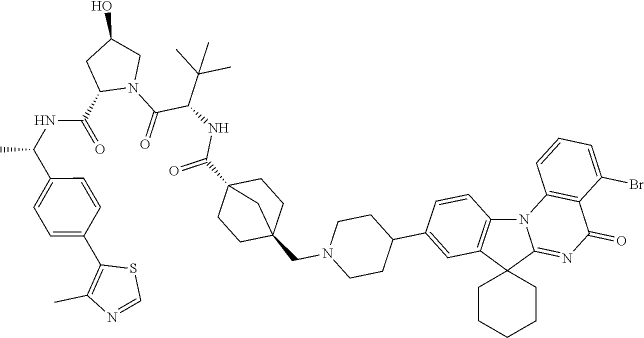 [SMILES: Cc1ncsc1-c1ccc([C@H](C)NC(=O)[C@@H]2C[C@@H](O)CN2C(=O)[C@@H](NC(=O)[C@]23CC[C@@](CN4CCC(c5ccc6c(c5)C5(CCCCC5)c5nc(=O)c7c(Br)cccc7n5-6)CC4)(CC2)C3)C(C)(C)C)cc1] | (2S,4R)-1-((S)-2-((1r,4S)-4-((4-(4′- bromo-5′-oxo-5′H-spiro[cyclohexane- 1,7′-indolo[1,2-a]quinazolin]-9′- yl)piperidin-1- yl)methyl)bicyclo[2.2.1]heptane-1- carboxamido)-3,3-dimethylbutanoyl)- 4-hydroxy-N-((S)-1-(4-(4- methylthiazol-5- yl)phenyl)ethyl)pyrrolidine-2- carboxamide |
| A20 | 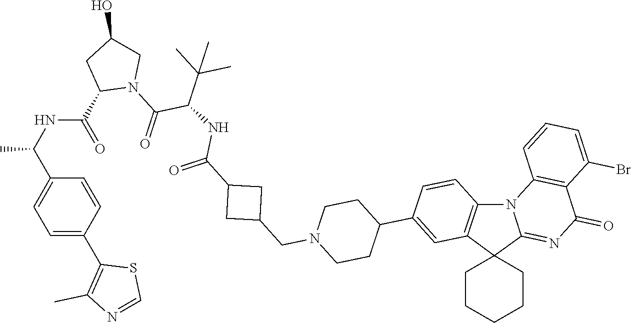 [SMILES: Cc1ncsc1-c1ccc([C@H](C)NC(=O)[C@@H]2C[C@@H](O)CN2C(=O)[C@@H](NC(=O)C2CC(CN3CCC(c4ccc5c(c4)C4(CCCCC4)c4nc(=O)c6c(Br)cccc6n4-5)CC3)C2)C(C)(C)C)cc1] | (2S,4R)-1-((S)-2-(3-((4-(4′-bromo-5′- oxo-5′H-spiro[cyclohexane-1,7′- indolo[1,2-a]quinazolin]-9′- yl)piperidin-1-yl)methyl)cyclobutane- 1-carboxamido)-3,3- dimethylbutanoyl)-4-hydroxy-N-((S)- 1-(4-(4-methylthiazol-5- yl)phenyl)ethyl)pyrrolidine-2- carboxamide |
| A21 | 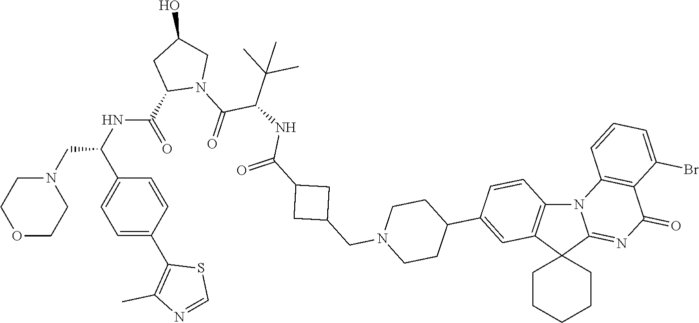 [SMILES: Cc1ncsc1-c1ccc([C@H](CN2CCOCC2)NC(=O)[C@@H]2C[C@@H](O)CN2C(=O)[C@@H](NC(=O)C2CC(CN3CCC(c4ccc5c(c4)C4(CCCCC4)c4nc(=O)c6c(Br)cccc6n4-5)CC3)C2)C(C)(C)C)cc1] | (2S,4R)-1-((S)-2-(3-((4-(4′-bromo-5′- oxo-5′H-spiro[cyclohexane-1,7′- indolo[1,2-a]quinazolin]-9′- yl)piperidin-1-yl)methyl)cyclobutane- 1-carboxamido)-3,3- dimethylbutanoyl)-4-hydroxy-N-((R)- 1-(4-(4-methylthiazol-5-yl)phenyl)-2- morpholinoethyl)pyrrolidine-2- carboxamide |
| A22 | 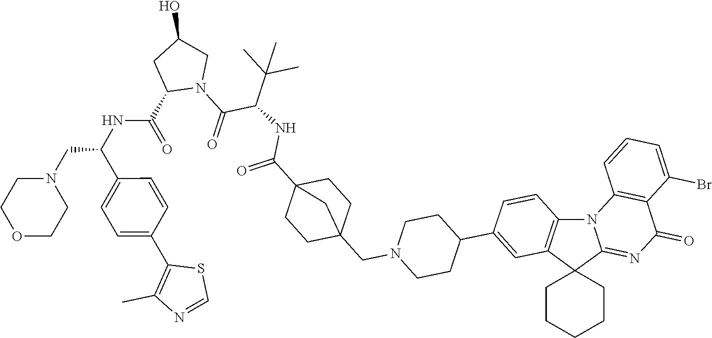 [SMILES: Cc1ncsc1-c1ccc([C@H](CN2CCOCC2)NC(=O)[C@@H]2C[C@@H](O)CN2C(=O)[C@@H](NC(=O)C23CCC(CN4CCC(c5ccc6c(c5)C5(CCCCC5)c5nc(=O)c7c(Br)cccc7n5-6)CC4)(CC2)C3)C(C)(C)C)cc1] | (2S,4R)-1-((S)-2-(4-((4-(4′-bromo-5′- oxo-5′H-spiro[cyclohexane-1,7′- indolo[1,2-a]quinazolin]-9′- yl)piperidin-1- yl)methyl)bicyclo[2.2.1]heptane-1- carboxamido)-3,3-dimethylbutanoyl)- 4-hydroxy-N-((R)-1-(4-(4- methylthiazol-5-yl)phenyl)-2- morpholinoethyl)pyrrolidine-2- carboxamide |
| A23 | 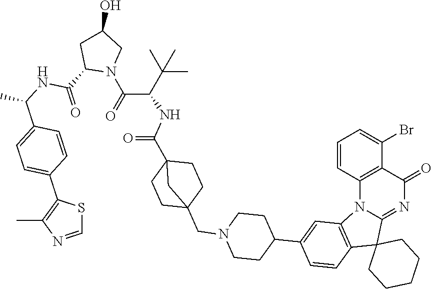 [SMILES: Cc1ncsc1-c1ccc([C@H](C)NC(=O)[C@@H]2C[C@@H](O)CN2C(=O)[C@@H](NC(=O)C23CCC(CN4CCC(c5ccc6c(c5)-n5c(nc(=O)c7c(Br)cccc75)C65CCCCC5)CC4)(CC2)C3)C(C)(C)C)cc1] | (2S,4R)-1-((S)-2-(4-((4-(4′-bromo-5′- oxo-5′H-spiro[cyclohexane-1,7′- indolo[1,2-a]quinazolin]-10′- yl)piperidin-1- yl)methyl)bicyclo[2.2.1]heptane-1- carboxamido)-3,3-dimethylbutanoyl)- 4-hydroxy-N-((S)-1-(4-(4- methylthiazol-5- yl)phenyl)ethyl)pyrrolidine-2- carboxamide |
| A24 | 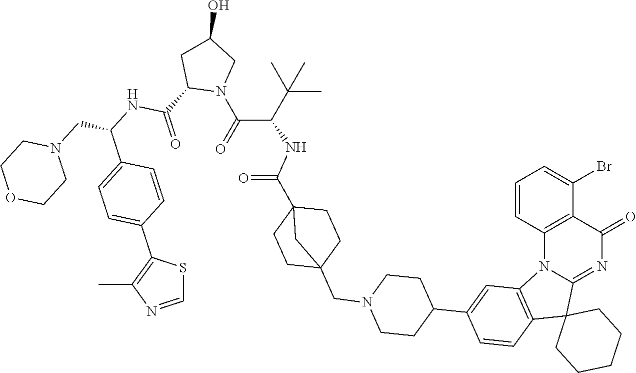 [SMILES: Cc1ncsc1-c1ccc([C@H](CN2CCOCC2)NC(=O)[C@@H]2C[C@@H](O)CN2C(=O)[C@@H](NC(=O)C23CCC(CN4CCC(c5ccc6c(c5)-n5c(nc(=O)c7c(Br)cccc75)C65CCCCC5)CC4)(CC2)C3)C(C)(C)C)cc1] | (2S,4R)-1-((S)-2-(4-((4-(4′-bromo-5′- oxo-5′H-spiro[cyclohexane-1,7′- indolo[1,2-a]quinazolin]-10′- yl)piperidin-1- yl)methyl)bicyclo[2.2.1]heptane-1- carboxamido)-3,3-dimethylbutanoyl)- 4-hydroxy-N-((R)-1-(4-(4- methylthiazol-5-yl)phenyl)-2- morpholinoethyl)pyrrolidine-2- carboxamide |
| A25 | 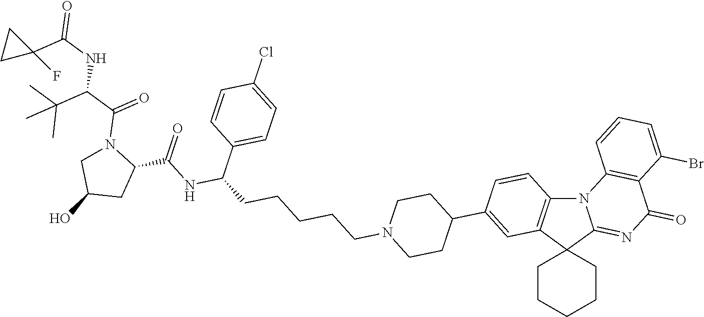 [SMILES: CC(C)(C)[C@H](NC(=O)C1(F)CC1)C(=O)N1C[C@H](O)C[C@H]1C(=O)N[C@@H](CCCCCN1CCC(c2ccc3c(c2)C2(CCCCC2)c2nc(=O)c4c(Br)cccc4n2-3)CC1)c1ccc(Cl)cc1] | (2S,4R)-N-((S)-6-(4-(4′-bromo-5′-oxo- 5′H-spiro[cyclohexane-1,7′-indolo[1,2- a]quinazolin]-9′-yl)piperidin-1-yl)-1- (4-chlorophenyl)hexyl)-1-((S)-2-(1- fluorocyclopropane-1-carboxamido)- 3,3-dimethylbutanoyl)-4- hydroxypyrrolidine-2-carboxamide |
| A26 | 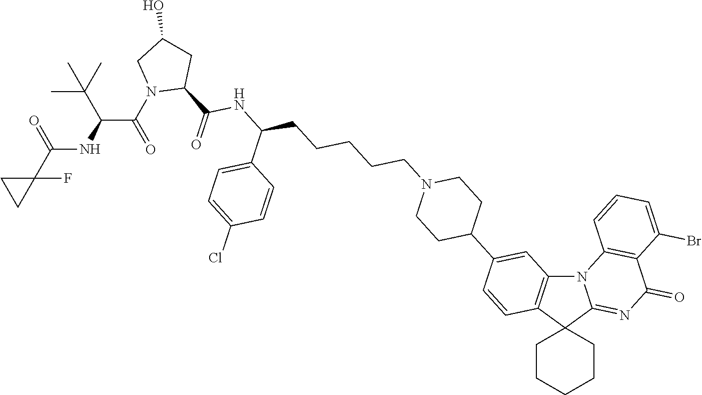 [SMILES: CC(C)(C)[C@H](NC(=O)C1(F)CC1)C(=O)N1C[C@H](O)C[C@H]1C(=O)N[C@@H](CCCCCN1CCC(c2ccc3c(c2)-n2c(nc(=O)c4c(Br)cccc42)C32CCCCC2)CC1)c1ccc(Cl)cc1] | (2S,4R)-N-((S)-6-(4-(4′-bromo-5′-oxo- 5′H-spiro[cyclohexane-1,7′-indolo[1,2- a]quinazolin]-10′-yl)piperidin-1-yl)-1- (4-chlorophenyl)hexyl)-1-((S)-2-(1- fluorocyclopropane-1-carboxamido)- 3,3-dimethylbutanoyl)-4- hydroxypyrrolidine-2-carboxamide |
| A27 | 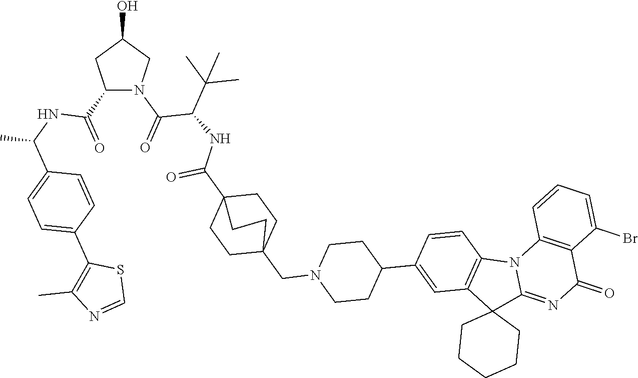 [SMILES: Cc1ncsc1-c1ccc([C@H](C)NC(=O)[C@@H]2C[C@@H](O)CN2C(=O)[C@@H](NC(=O)C23CCC(CN4CCC(c5ccc6c(c5)C5(CCCCC5)c5nc(=O)c7c(Br)cccc7n5-6)CC4)(CC2)CC3)C(C)(C)C)cc1] | (2S,4R)-1-((S)-2-(4-((4-(4′-bromo-5′- oxo-5′H-spiro[cyclohexane-1,7′- indolo[1,2-a]quinazolin]-9′- yl)piperidin-1- yl)methyl)bicyclo[2.2.2]octane-1- carboxamido)-3,3-dimethylbutanoyl)- 4-hydroxy-N-((S)-1-(4-(4- methylthiazol-5- yl)phenyl)ethyl)pyrrolidine-2- carboxamide |
| A28 | 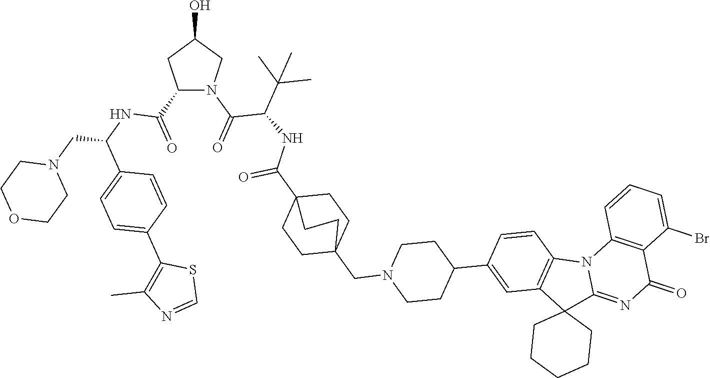 [SMILES: Cc1ncsc1-c1ccc([C@H](CN2CCOCC2)NC(=O)[C@@H]2C[C@@H](O)CN2C(=O)[C@@H](NC(=O)C23CCC(CN4CCC(c5ccc6c(c5)C5(CCCCC5)c5nc(=O)c7c(Br)cccc7n5-6)CC4)(CC2)CC3)C(C)(C)C)cc1] | (2S,4R)-1-((S)-2-(4-((4-(4′-bromo-5′- oxo-5′H-spiro[cyclohexane-1,7′- indolo[1,2-a]quinazolin]-9′- yl)piperidin-1- yl)methyl)bicyclo[2.2.2]octane-1- carboxamido)-3,3-dimethylbutanoyl)- 4-hydroxy-N-((R)-1-(4-(4- methylthiazol-5-yl)phenyl)-2- morpholinoethyl)pyrrolidine-2- carboxamide |
| A29 | 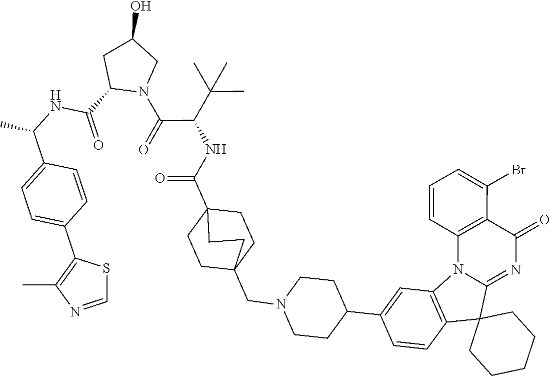 [SMILES: Cc1ncsc1-c1ccc([C@H](C)NC(=O)[C@@H]2C[C@@H](O)CN2C(=O)[C@@H](NC(=O)C23CCC(CN4CCC(c5ccc6c(c5)-n5c(nc(=O)c7c(Br)cccc75)C65CCCCC5)CC4)(CC2)CC3)C(C)(C)C)cc1] | (2S,4R)-1-((S)-2-(4-((4-(4′-bromo-5′- oxo-5′H-spiro[cyclohexane-1,7′- indolo[1,2-a]quinazolin]-10′- yl)piperidin-1- yl)methyl)bicyclo[2.2.2]octane-1- carboxamido)-3,3-dimethylbutanoyl)- 4-hydroxy-N-((S)-1-(4-(4- methylthiazol-5- yl)phenyl)ethyl)pyrrolidine-2- carboxamide |
| A30 | 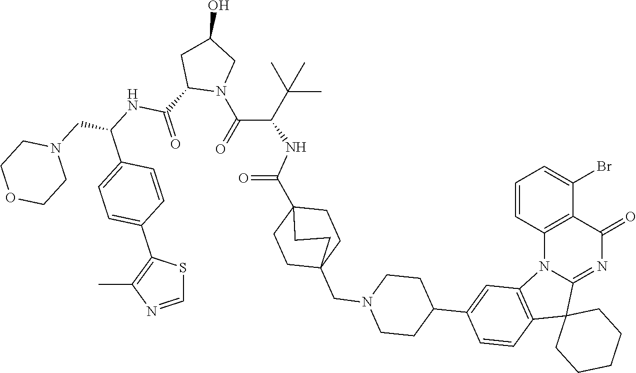 [SMILES: Cc1ncsc1-c1ccc([C@H](CN2CCOCC2)NC(=O)[C@@H]2C[C@@H](O)CN2C(=O)[C@@H](NC(=O)C23CCC(CN4CCC(c5ccc6c(c5)-n5c(nc(=O)c7c(Br)cccc75)C65CCCCC5)CC4)(CC2)CC3)C(C)(C)C)cc1] | (2S,4R)-1-((S)-2-(4-((4-(4′-bromo-5′- oxo-5′H-spiro[cyclohexane-1,7′- indolo[1,2-a]quinazolin]-10′- yl)piperidin-1- yl)methyl)bicyclo[2.2.2]octane-1- carboxamido)-3,3-dimethylbutanoyl)- 4-hydroxy-N-((R)-1-(4-(4- methylthiazol-5-yl)phenyl)-2- morpholinoethyl)pyrrolidine-2- carboxamide |
| A31 | 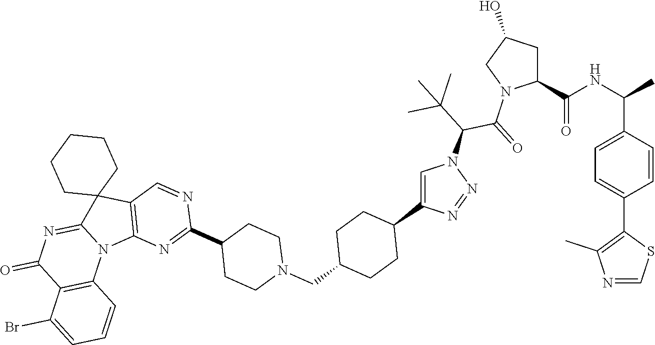 | (2S,4R)-1-((S)-2-(4-((1r,4S)-4-((4-(4′- bromo-5′-oxo-5′H-spiro[cyclohexane- 1,7′-pyrimido[5′,4′:4,5]pyrrolo[1,2- a]quinazolin]-10′-yl)piperidin-1- yl)methyl)cyclohexyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A32 | 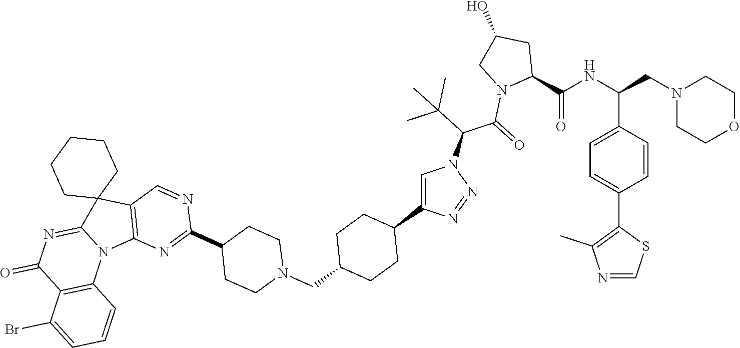 | (2S,4R)-1-((S)-2-(4-((1r,4S)-4-((4-(4′- bromo-5′-oxo-5′H-spiro[cyclohexane- 1,7′-pyrimido[5′,4′:4,5]pyrrolo[1,2- a]quinazolin]-10′-yl)piperidin-1- yl)methyl)cyclohexyl)-1H-1,2,3- triazol-1-yl)-3,3-dimethylbutanoyl)-4- hydroxy-N-((R)-1-(4-(4-methylthiazol- 5-yl)phenyl)-2- morpholinoethyl)pyrrolidine-2- carboxamide |
| A33 | 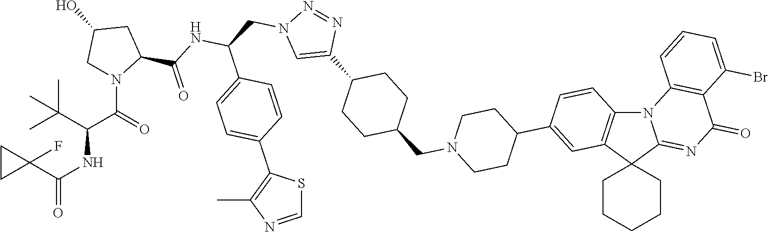 [SMILES: Cc1ncsc1-c1ccc([C@H](Cn2cc([C@H]3CC[C@H](CN4CCC(c5ccc6c(c5)C5(CCCCC5)c5nc(=O)c7c(Br)cccc7n5-6)CC4)CC3)nn2)NC(=O)[C@@H]2C[C@@H](O)CN2C(=O)[C@@H](NC(=O)C2(F)CC2)C(C)(C)C)cc1] | (2S,4R)-N-((R)-2-(4-((1r,4R)-4-((4-(4′- bromo-5′-oxo-5′H-spiro[cyclohexane- 1,7′-indolo[1,2-a]quinazolin]-9′- yl)piperidin-1-yl)methyl)cyclohexyl)- 1H-1,2,3-triazol-1-yl)-1-(4-(4- methylthiazol-5-yl)phenyl)ethyl)-1- ((S)-2-(1-fluorocyclopropane-1- carboxamido)-3,3-dimethylbutanoyl)- 4-hydroxypyrrolidine-2-carboxamide |
| A34 | 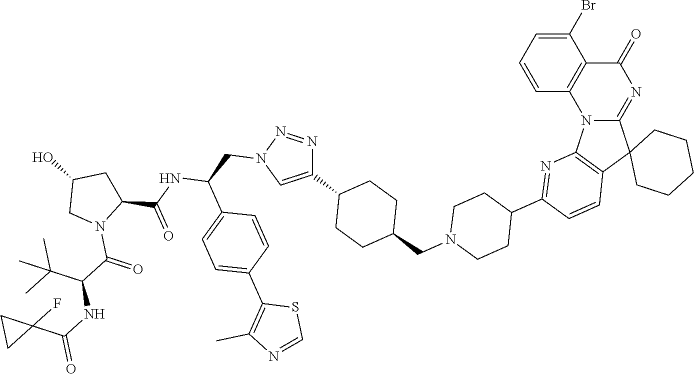 [SMILES: Cc1ncsc1-c1ccc([C@H](Cn2cc([C@H]3CC[C@H](CN4CCC(c5ccc6c(n5)-n5c(nc(=O)c7c(Br)cccc75)C65CCCCC5)CC4)CC3)nn2)NC(=O)[C@@H]2C[C@@H](O)CN2C(=O)[C@@H](NC(=O)C2(F)CC2)C(C)(C)C)cc1] | (2S,4R)-N-((R)-2-(4-((1r,4R)-4-((4-(4′- bromo-5′-oxo-5′H-spiro[cyclohexane- 1,7′-pyrido[3′,2′:4,5]pyrrolo[1,2- a]quinazolin]-10′-yl)piperidin-1- yl)methyl)cyclohexyl)-1H-1,2,3- triazol-1-yl)-1-(4-(4-methylthiazol-5- yl)phenyl)ethyl)-1-((S)-2-(1- fluorocyclopropane-1-carboxamido)- 3,3-dimethylbutanoyl)-4- hydroxypyrrolidine-2-carboxamide |
| A35 | 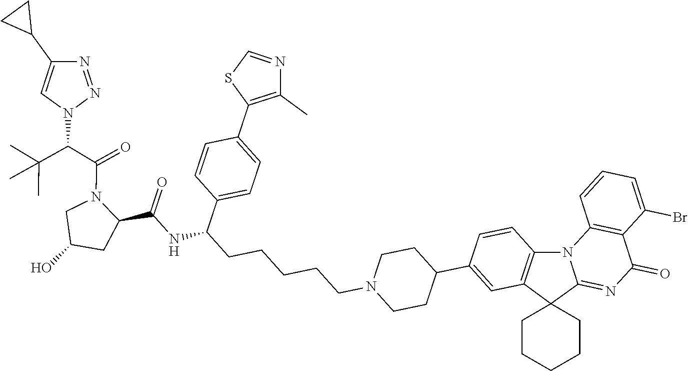 [SMILES: Cc1ncsc1-c1ccc([C@H](CCCCCN2CCC(c3ccc4c(c3)C3(CCCCC3)c3nc(=O)c5c(Br)cccc5n3-4)CC2)NC(=O)[C@H]2C[C@H](O)CN2C(=O)[C@@H](n2cc(C3CC3)nn2)C(C)(C)C)cc1] | (2R,4S)-N-((S)-6-(4-(4′-bromo-5′-oxo- 5′H-spiro[cyclohexane-1,7′-indolo[1,2- a]quinazolin]-9′-yl)piperidin-1-yl)-1- (4-(4-methylthiazol-5- yl)phenyl)hexyl)-1-((S)-2-(4- cyclopropyl-1H-1,2,3-triazol-1-yl)-3,3- dimethylbutanoyl)-4- hydroxypyrrolidine-2-carboxamide |
| A36 | 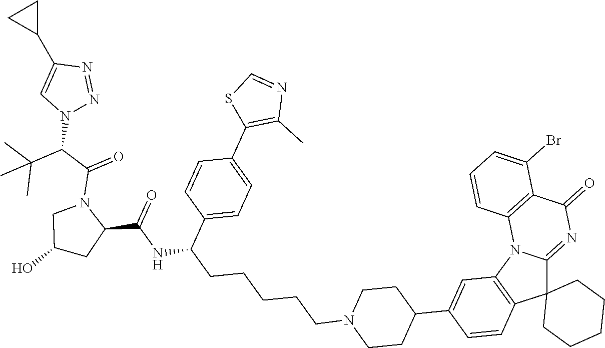 [SMILES: Cc1ncsc1-c1ccc([C@H](CCCCCN2CCC(c3ccc4c(c3)-n3c(nc(=O)c5c(Br)cccc53)C43CCCCC3)CC2)NC(=O)[C@H]2C[C@H](O)CN2C(=O)[C@@H](n2cc(C3CC3)nn2)C(C)(C)C)cc1] | (2R,4S)-N-((S)-6-(4-(4′-bromo-5′-oxo- 5′H-spiro[cyclohexane-1,7′-indolo[1,2- a]quinazolin]-10′-yl)piperidin-1-yl)-1- (4-(4-methylthiazol-5- yl)phenyl)hexyl)-1-((S)-2-(4- cyclopropyl-1H-1,2,3-triazol-1-yl)-3,3- dimethylbutanoyl)-4- hydroxypyrrolidine-2-carboxamide |
| A37 | 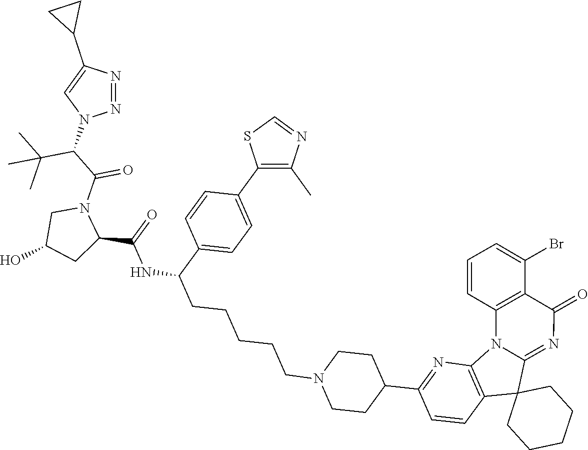 [SMILES: Cc1ncsc1-c1ccc([C@H](CCCCCN2CCC(c3ccc4c(n3)-n3c(nc(=O)c5c(Br)cccc53)C43CCCCC3)CC2)NC(=O)[C@H]2C[C@H](O)CN2C(=O)[C@@H](n2cc(C3CC3)nn2)C(C)(C)C)cc1] | (2R,4S)-N-((S)-6-(4-(4′-bromo-5′-oxo- 5′H-spiro[cyclohexane-1,7′- pyrido[3′,2′:4,5]pyrrolo[1,2- a]quinazolin]-10′-yl)piperidin-1-yl)-1- (4-(4-methylthiazol-5- yl)phenyl)hexyl)-1-((S)-2-(4- cyclopropyl-1H-1,2,3-triazol-1-yl)-3,3- dimethylbutanoyl)-4- hydroxypyrrolidine-2-carboxamide |
| A38 | 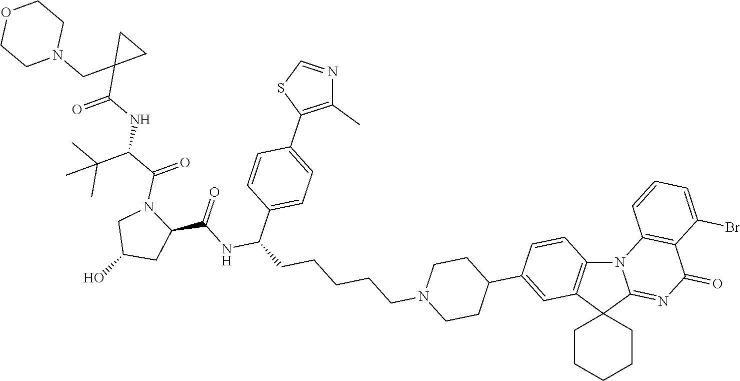 [SMILES: Cc1ncsc1-c1ccc([C@H](CCCCCN2CCC(c3ccc4c(c3)C3(CCCCC3)c3nc(=O)c5c(Br)cccc5n3-4)CC2)NC(=O)[C@H]2C[C@H](O)CN2C(=O)[C@@H](NC(=O)C2(CN3CCOCC3)CC2)C(C)(C)C)cc1] | (2R,4S)-N-((S)-6-(4-(4′-bromo-5′-oxo- 5′H-spiro[cyclohexane-1,7′-indolo[1,2- a]quinazolin]-9′-yl)piperidin-1-yl)-1- (4-(4-methylthiazol-5- yl)phenyl)hexyl)-1-((S)-3,3-dimethyl- 2-(1-(morpholinomethyl)cyclopropane- 1-carboxamido)butanoyl)-4- hydroxypyrrolidine-2-carboxamide |
| A39 | 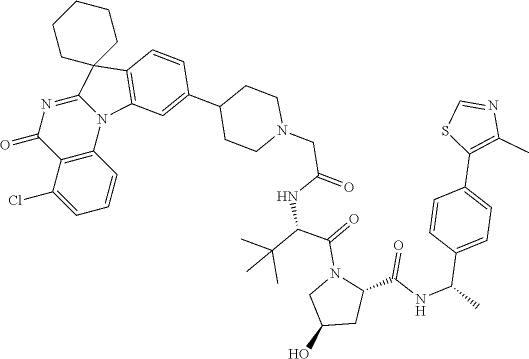 [SMILES: Cc1ncsc1-c1ccc([C@H](C)NC(=O)[C@@H]2C[C@@H](O)CN2C(=O)[C@@H](NC(=O)CN2CCC(c3ccc4c(c3)-n3c(nc(=O)c5c(Cl)cccc53)C43CCCCC3)CC2)C(C)(C)C)cc1] | (2S,4R)-1-((S)-2-(2-(4-(4′-chloro-5′- oxo-5′H-spiro[cyclohexane-1,7′- indolo[1,2-a]quinazolin]-10′- yl)piperidin-1-yl)acetamido)-3,3- dimethylbutanoyl)-4-hydroxy-N-((S)- 1-(4-(4-methylthiazol-5- yl)phenyl)ethyl)pyrrolidine-2- carboxamide |
| A40 | 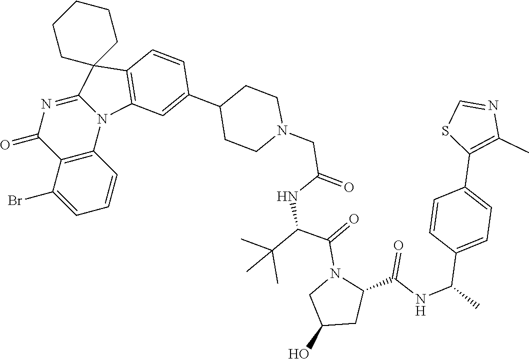 [SMILES: Cc1ncsc1-c1ccc([C@H](C)NC(=O)[C@@H]2C[C@@H](O)CN2C(=O)[C@@H](NC(=O)CN2CCC(c3ccc4c(c3)-n3c(nc(=O)c5c(Br)cccc53)C43CCCCC3)CC2)C(C)(C)C)cc1] | (2S,4R)-1-((S)-2-(2-(4-(4′-bromo-5′- oxo-5′H-spiro[cyclohexane-1,7′- indolo[1,2-a]quinazolin]-10′- yl)piperidin-1-yl)acetamido)-3,3- dimethylbutanoyl)-4-hydroxy-N-((S)- 1-(4-(4-methylthiazol-5- yl)phenyl)ethyl)pyrrolidine-2- carboxamide |
| A41 | 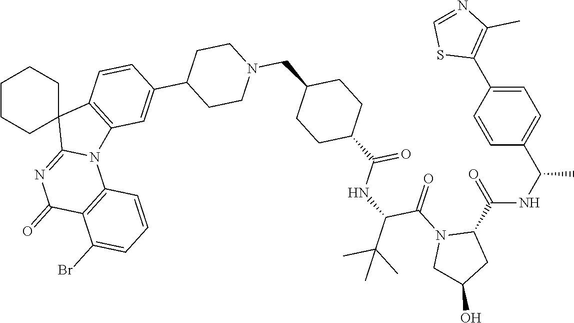 [SMILES: Cc1ncsc1-c1ccc([C@H](C)NC(=O)[C@@H]2C[C@@H](O)CN2C(=O)[C@@H](NC(=O)[C@H]2CC[C@H](CN3CCC(c4ccc5c(c4)-n4c(nc(=O)c6c(Br)cccc64)C54CCCCC4)CC3)CC2)C(C)(C)C)cc1] | (2S,4R)-1-((S)-2-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexane- 1-carboxamido)-3,3- dimethylbutanoyl)-4-hydroxy-N-((S)- 1-(4-(4-methylthiazol-5- yl)phenyl)ethyl)pyrrolidine-2- carboxamide |
| A42 | 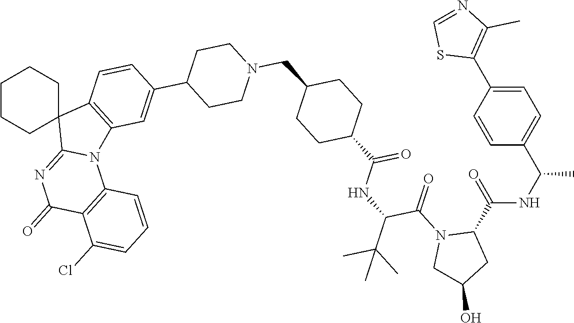 [SMILES: Cc1ncsc1-c1ccc([C@H](C)NC(=O)[C@@H]2C[C@@H](O)CN2C(=O)[C@@H](NC(=O)[C@H]2CC[C@H](CN3CCC(c4ccc5c(c4)-n4c(nc(=O)c6c(Cl)cccc64)C54CCCCC4)CC3)CC2)C(C)(C)C)cc1] | (2S,4R)-1-((S)-2-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexane- 1-carboxamido)-3,3- dimethylbutanoyl)-4-hydroxy-N-((S)- 1-(4-(4-methylthiazol-5- yl)phenyl)ethyl)pyrrolidine-2- carboxamide |
| A43 | 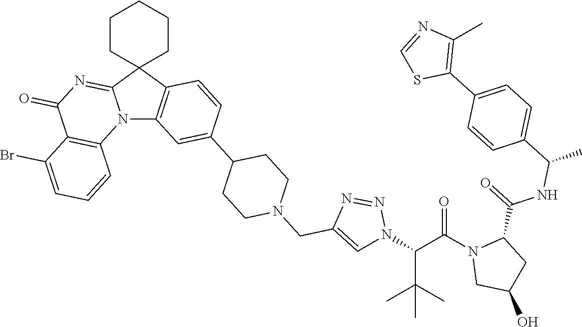 [SMILES: Cc1ncsc1-c1ccc([C@H](C)NC(=O)[C@@H]2C[C@@H](O)CN2C(=O)[C@@H](n2cc(CN3CCC(c4ccc5c(c4)-n4c(nc(=O)c6c(Br)cccc64)C54CCCCC4)CC3)nn2)C(C)(C)C)cc1] | (2S,4R)-1-((S)-2-(4-((4-(4′-bromo-5′- oxo-5′H-spiro[cyclohexane-1,7′- indolo[1,2-a]quinazolin]-10′- yl)piperidin-1-yl)methyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A44 | 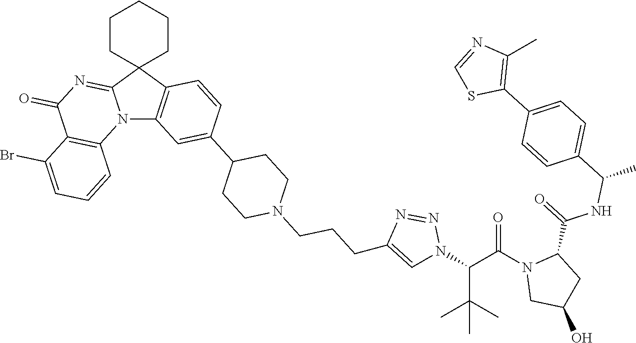 [SMILES: Cc1ncsc1-c1ccc([C@H](C)NC(=O)[C@@H]2C[C@@H](O)CN2C(=O)[C@@H](n2cc(CCCN3CCC(c4ccc5c(c4)-n4c(nc(=O)c6c(Br)cccc64)C54CCCCC4)CC3)nn2)C(C)(C)C)cc1] | (2S,4R)-1-((S)-2-(4-(3-(4-(4′-bromo-5′- oxo-5′H-spiro[cyclohexane-1,7′- indolo[1,2-a]quinazolin]-10′- yl)piperidin-1-yl)propyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A45 | 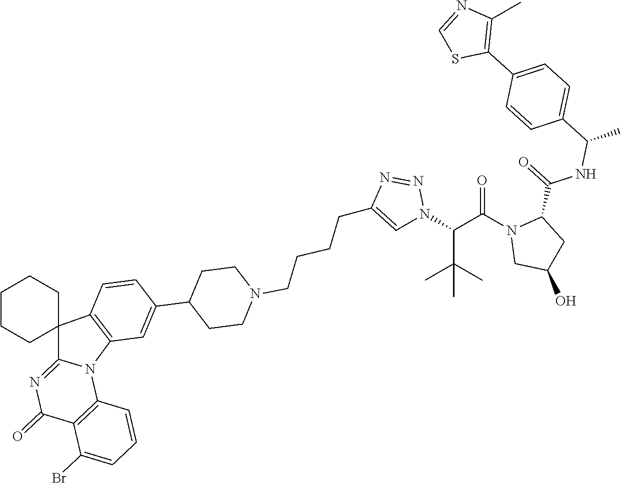 [SMILES: Cc1ncsc1-c1ccc([C@H](C)NC(=O)[C@@H]2C[C@@H](O)CN2C(=O)[C@@H](n2cc(CCCCN3CCC(c4ccc5c(c4)-n4c(nc(=O)c6c(Br)cccc64)C54CCCCC4)CC3)nn2)C(C)(C)C)cc1] | (2S,4R)-1-((S)-2-(4-(4-(4-(4′-bromo-5′- oxo-5′H-spiro[cyclohexane-1,7′- indolo[1,2-a]quinazolin]-10′- yl)piperidin-1-yl)butyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A46 | 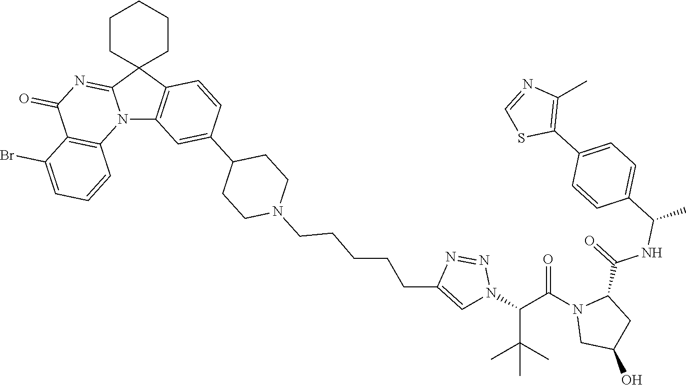 [SMILES: Cc1ncsc1-c1ccc([C@H](C)NC(=O)[C@@H]2C[C@@H](O)CN2C(=O)[C@@H](n2cc(CCCCCN3CCC(c4ccc5c(c4)-n4c(nc(=O)c6c(Br)cccc64)C54CCCCC4)CC3)nn2)C(C)(C)C)cc1] | (2S,4R)-1-((S)-2-(4-(5-(4-(4′-bromo-5′- oxo-5′H-spiro[cyclohexane-1,7′- indolo[1,2-a]quinazolin]-10′- yl)piperidin-1-yl)pentyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A47 | 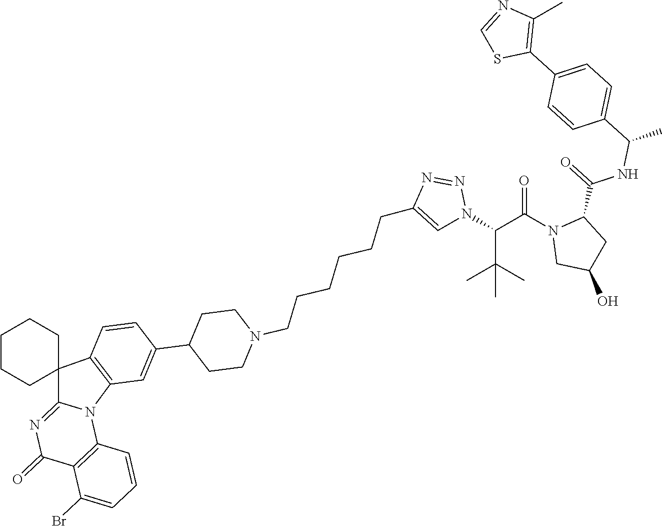 [SMILES: Cc1ncsc1-c1ccc([C@H](C)NC(=O)[C@@H]2C[C@@H](O)CN2C(=O)[C@@H](n2cc(CCCCCCN3CCC(c4ccc5c(c4)-n4c(nc(=O)c6c(Br)cccc64)C54CCCCC4)CC3)nn2)C(C)(C)C)cc1] | (2S,4R)-1-((S)-2-(4-(6-(4-(4′-bromo-5′- oxo-5′H-spiro[cyclohexane-1,7′- indolo[1,2-a]quinazolin]-10′- yl)piperidin-1-yl)hexyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A48 | 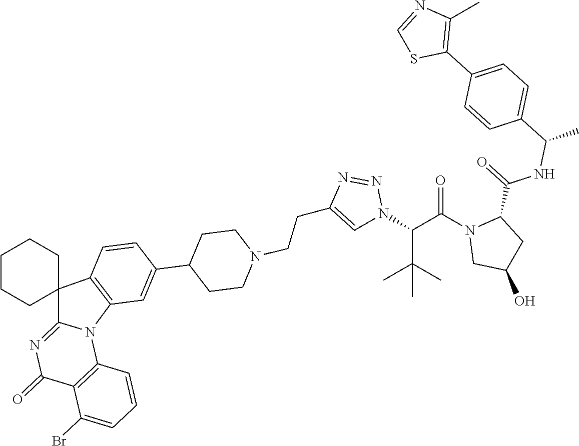 [SMILES: Cc1ncsc1-c1ccc([C@H](C)NC(=O)[C@@H]2C[C@@H](O)CN2C(=O)[C@@H](n2cc(CCN3CCC(c4ccc5c(c4)-n4c(nc(=O)c6c(Br)cccc64)C54CCCCC4)CC3)nn2)C(C)(C)C)cc1] | (2S,4R)-1-((S)-2-(4-(2-(4-(4′-bromo-5′- oxo-5′H-spiro[cyclohexane-1,7′- indolo[1,2-a]quinazolin]-10′- yl)piperidin-1-yl)ethyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A49 | 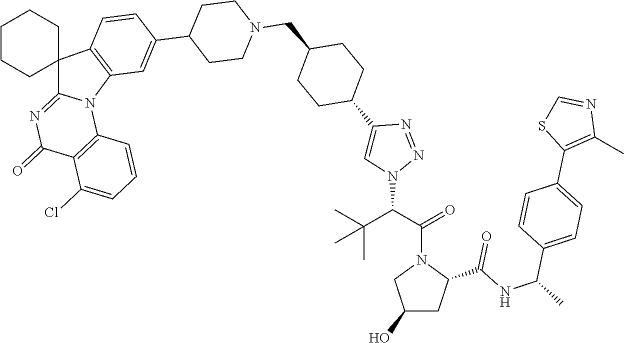 [SMILES: Cc1ncsc1-c1ccc([C@H](C)NC(=O)[C@@H]2C[C@@H](O)CN2C(=O)[C@@H](n2cc([C@H]3CC[C@H](CN4CCC(c5ccc6c(c5)-n5c(nc(=O)c7c(Cl)cccc75)C65CCCCC5)CC4)CC3)nn2)C(C)(C)C)cc1] | (2S,4R)-1-((S)-2-(4-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((S)- 1-(4-(4-methylthiazol-5- yl)phenyl)ethyl)pyrrolidine-2- carboxamide |
| A50 | 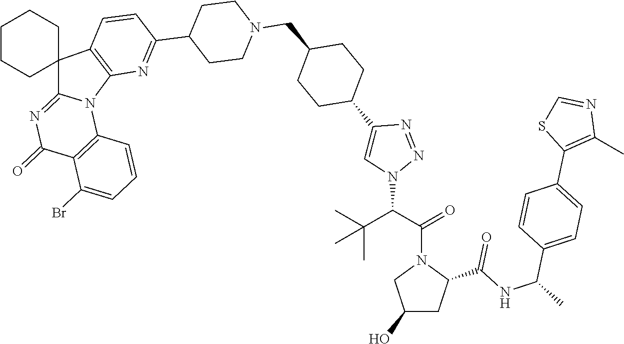 [SMILES: Cc1ncsc1-c1ccc([C@H](C)NC(=O)[C@@H]2C[C@@H](O)CN2C(=O)[C@@H](n2cc([C@H]3CC[C@H](CN4CCC(c5ccc6c(n5)-n5c(nc(=O)c7c(Br)cccc75)C65CCCCC5)CC4)CC3)nn2)C(C)(C)C)cc1] | (2S,4R)-1-((S)-2-(4-((1r,4S)-4-((4-(4′- bromo-5′-oxo-5′H-spiro[cyclohexane- 1,7′-pyrido[3′,2′:4,5]pyrrolo[1,2- a]quinazolin]-10′-yl)piperidin-1- yl)methyl)cyclohexyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A51 | 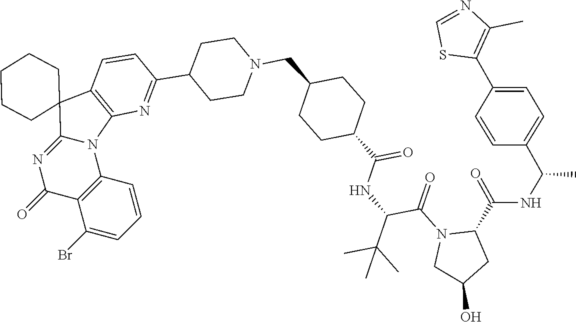 [SMILES: Cc1ncsc1-c1ccc([C@H](C)NC(=O)[C@@H]2C[C@@H](O)CN2C(=O)[C@@H](NC(=O)[C@H]2CC[C@H](CN3CCC(c4ccc5c(n4)-n4c(nc(=O)c6c(Br)cccc64)C54CCCCC4)CC3)CC2)C(C)(C)C)cc1] | (2S,4R)-1-((S)-2-((1r,4S)-4-((4-(4′- bromo-5′-oxo-5′H-spiro[cyclohexane- 1,7′-pyrido[3′,2′:4,5]pyrrolo[1,2- a]quinazolin]-10′-yl)piperidin-1- yl)methyl)cyclohexane-1- carboxamido)-3,3-dimethylbutanoyl)- 4-hydroxy-N-((S)-1-(4-(4- methylthiazol-5- yl)phenyl)ethyl)pyrrolidine-2- carboxamide |
| A52 | 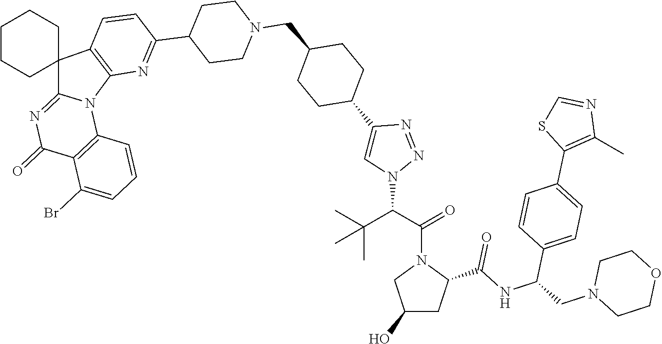 [SMILES: Cc1ncsc1-c1ccc([C@H](CN2CCOCC2)NC(=O)[C@@H]2C[C@@H](O)CN2C(=O)[C@@H](n2cc([C@H]3CC[C@H](CN4CCC(c5ccc6c(n5)-n5c(nc(=O)c7c(Br)cccc75)C65CCCCC5)CC4)CC3)nn2)C(C)(C)C)cc1] | (2S,4R)-1-((S)-2-(4-((1r,4S)-4-((4-(4′- bromo-5′-oxo-5′H-spiro[cyclohexane- 1,7′-pyrido[3′,2′:4,5]pyrrolo[1,2- a]quinazolin]-10′-yl)piperidin-1- yl)methyl)cyclohexyl)-1H-1,2,3- triazol-1-yl)-3,3-dimethylbutanoyl)-4- hydroxy-N-((R)-1-(4-(4-methylthiazol- 5-yl)phenyl)-2- morpholinoethyl)pyrrolidine-2- carboxamide |
| A53 | 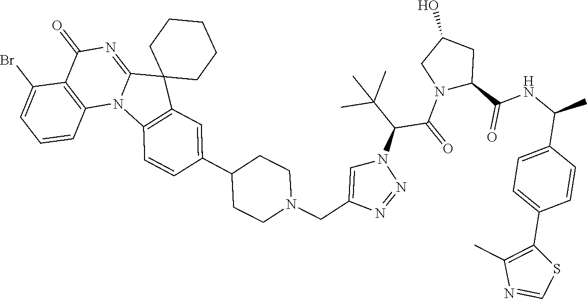 [SMILES: Cc1ncsc1-c1ccc([C@H](C)NC(=O)[C@@H]2C[C@@H](O)CN2C(=O)[C@@H](n2cc(CN3CCC(c4ccc5c(c4)C4(CCCCC4)c4nc(=O)c6c(Br)cccc6n4-5)CC3)nn2)C(C)(C)C)cc1] | (2S,4R)-1-((S)-2-(4-((4-(4′-bromo-5′- oxo-5′H-spiro[cyclohexane-1,7′- indolo[1,2-a]quinazolin]-9′- yl)piperidin-1-yl)methyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A54 | 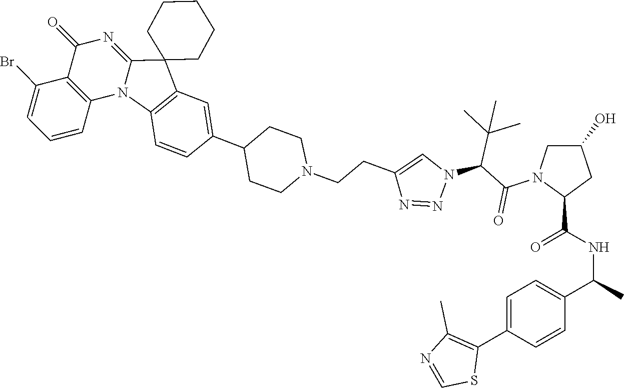 [SMILES: Cc1ncsc1-c1ccc([C@H](C)NC(=O)[C@@H]2C[C@@H](O)CN2C(=O)[C@@H](n2cc(CCN3CCC(c4ccc5c(c4)C4(CCCCC4)c4nc(=O)c6c(Br)cccc6n4-5)CC3)nn2)C(C)(C)C)cc1] | (2S,4R)-1-((S)-2-(4-(2-(4-(4′-bromo-5′- oxo-5′H-spiro[cyclohexane-1,7′- indolo[1,2-a]quinazolin]-9′- yl)piperidin-1-yl)ethyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A55 | 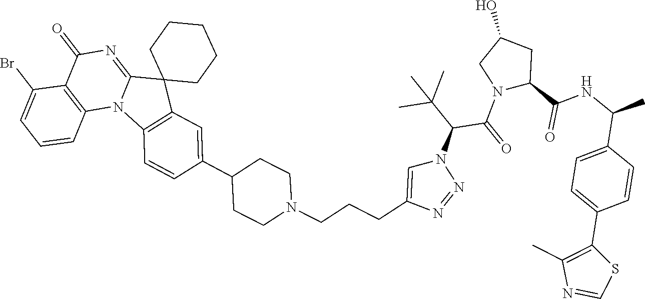 [SMILES: Cc1ncsc1-c1ccc([C@H](C)NC(=O)[C@@H]2C[C@@H](O)CN2C(=O)[C@@H](n2cc(CCCN3CCC(c4ccc5c(c4)C4(CCCCC4)c4nc(=O)c6c(Br)cccc6n4-5)CC3)nn2)C(C)(C)C)cc1] | (2S,4R)-1-((S)-2-(4-(3-(4-(4′-bromo-5′- oxo-5′H-spiro[cyclohexane-1,7′- indolo[1,2-a]quinazolin]-9′- yl)piperidin-1-yl)propyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A56 | 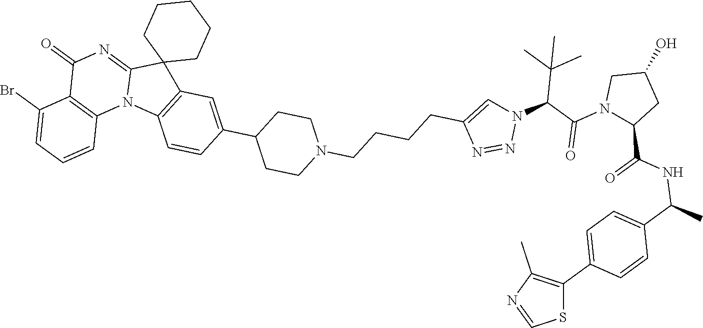 [SMILES: Cc1ncsc1-c1ccc([C@H](C)NC(=O)[C@@H]2C[C@@H](O)CN2C(=O)[C@@H](n2cc(CCCCN3CCC(c4ccc5c(c4)C4(CCCCC4)c4nc(=O)c6c(Br)cccc6n4-5)CC3)nn2)C(C)(C)C)cc1] | (2S,4R)-1-((S)-2-(4-(4-(4-(4′-bromo-5′- oxo-5′H-spiro[cyclohexane-1,7′- indolo[1,2-a]quinazolin]-9′- yl)piperidin-1-yl)butyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A57 | 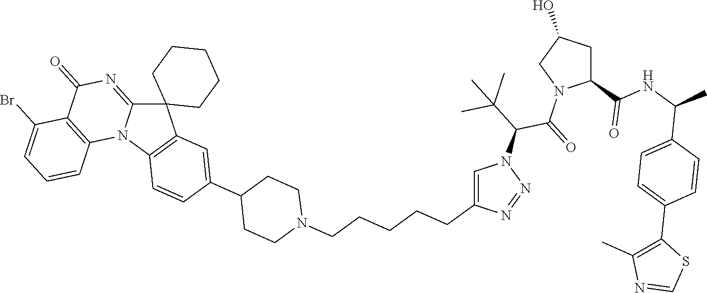 [SMILES: Cc1ncsc1-c1ccc([C@H](C)NC(=O)[C@@H]2C[C@@H](O)CN2C(=O)[C@@H](n2cc(CCCCCN3CCC(c4ccc5c(c4)C4(CCCCC4)c4nc(=O)c6c(Br)cccc6n4-5)CC3)nn2)C(C)(C)C)cc1] | (2S,4R)-1-((S)-2-(4-(5-(4-(4′-bromo-5′- oxo-5′H-spiro[cyclohexane-1,7′- indolo[1,2-a]quinazolin]-9′- yl)piperidin-1-yl)pentyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A58 | 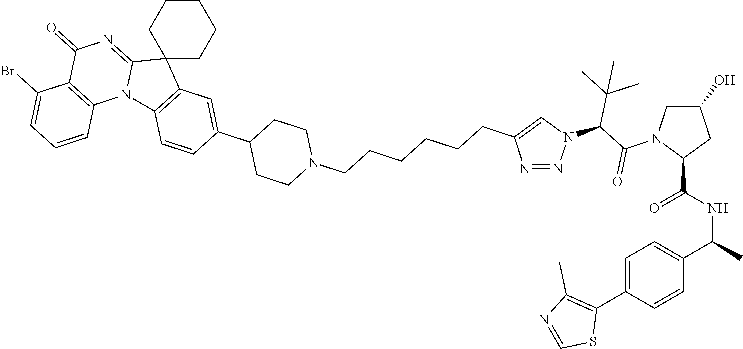 [SMILES: Cc1ncsc1-c1ccc([C@H](C)NC(=O)[C@@H]2C[C@@H](O)CN2C(=O)[C@@H](n2cc(CCCCCCN3CCC(c4ccc5c(c4)C4(CCCCC4)c4nc(=O)c6c(Br)cccc6n4-5)CC3)nn2)C(C)(C)C)cc1] | (2S,4R)-1-((S)-2-(4-(6-(4-(4′-bromo-5′- oxo-5′H-spiro[cyclohexane-1,7′- indolo[1,2-a]quinazolin]-9′- yl)piperidin-1-yl)hexyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A59 | 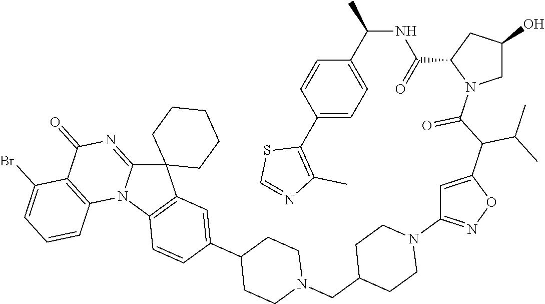 [SMILES: Cc1ncsc1-c1ccc([C@@H](C)NC(=O)[C@@H]2C[C@@H](O)CN2C(=O)C(c2cc(N3CCC(CN4CCC(c5ccc6c(c5)C5(CCCCC5)c5nc(=O)c7c(Br)cccc7n5-6)CC4)CC3)no2)C(C)C)cc1] | (2S,4R)-1-(2-(3-(4-((4-(4′-bromo-5′- oxo-5′H-spiro[cyclohexane-1,7′- indolo[1,2-a]quinazolin]-9′- yl)piperidin-1-yl)methyl)piperidin-1- yl)isoxazol-5-yl)-3-methylbutanoyl)-4- hydroxy-N-((R)-1-(4-(4-methylthiazol- 5-yl)phenyl)ethyl)pyrrolidine-2- carboxamide |
| A60 | 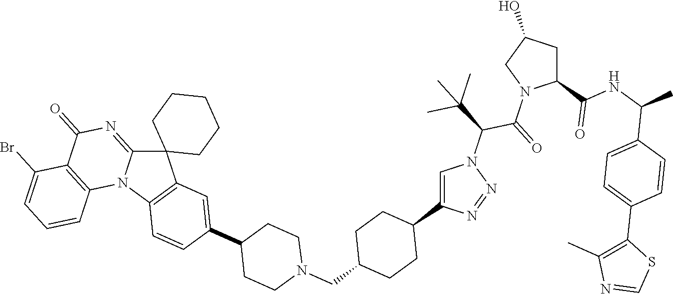 | (2S,4R)-1-((S)-2-(4-((1r,4S)-4-((4-(4′- bromo-5′-oxo-5′H-spiro[cyclohexane- 1,7′-indolo[1,2-a]quinazolin]-9′- yl)piperidin-1-yl)methyl)cyclohexyl)- 1H-1,2,3-triazol-1-yl)-3,3- dimethylbutanoyl)-4-hydroxy-N-((S)- 1-(4-(4-methylthiazol-5- yl)phenyl)ethyl)pyrrolidine-2- carboxamide |
| A61 | 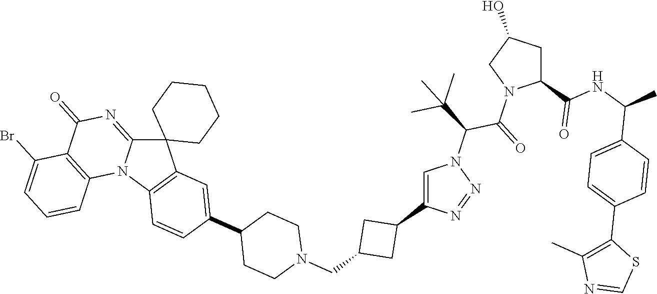 | (2S,4R)-1-((S)-2-(4-((1r,3S)-3-((4-(4′- bromo-5′-oxo-5′H-spiro[cyclohexane- 1,7′-indolo[1,2-a]quinazolin]-9′- yl)piperidin-1-yl)methyl)cyclobutyl)- 1H-1,2,3-triazol-1-yl)-3,3- dimethylbutanoyl)-4-hydroxy-N-((S)- 1-(4-(4-methylthiazol-5- yl)phenyl)ethyl)pyrrolidine-2- carboxamide |
| A62 | 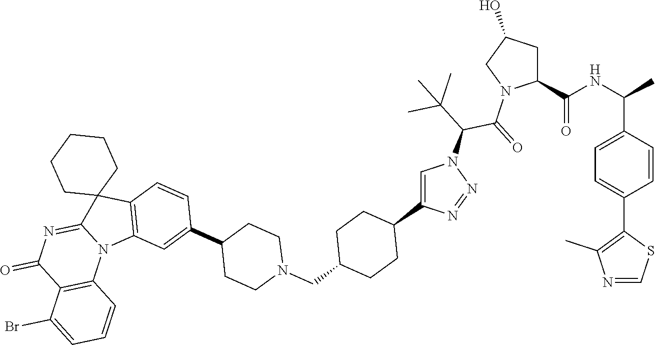 | (2S,4R)-1-((S)-2-(4-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((S)- 1-(4-(4-methylthiazol-5- yl)phenyl)ethyl)pyrrolidine-2- carboxamide |
| A63 | 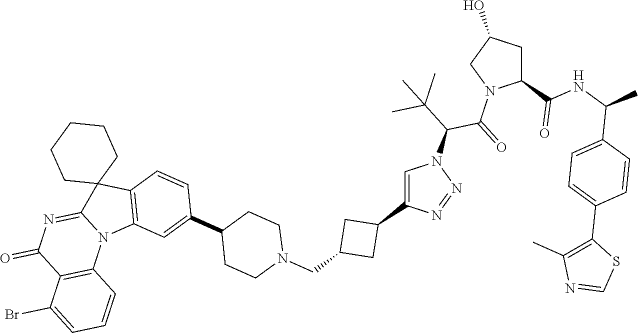 | (2S,4R)-1-((S)-2-(4-((1r,3S)-3-((4-(4′- bromo-5′-oxo-5′H-spiro[cyclohexane- 1,7′-indolo[1,2-a]quinazolin]-10′- yl)piperidin-1-yl)methyl)cyclobutyl)- 1H-1,2,3-triazol-1-yl)-3,3- dimethylbutanoyl)-4-hydroxy-N-((S)- 1-(4-(4-methylthiazol-5- yl)phenyl)ethyl)pyrrolidine-2- carboxamide |
| A64 | 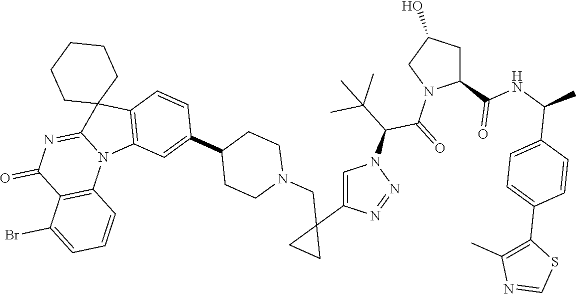 | (2S,4R)-1-((S)-2-(4-(1-((4-(4′-bromo- 5′-oxo-5′H-spiro[cyclohexane-1,7′- indolo[1,2-a]quinazolin]-10′- yl)piperidin-1-yl)methyl)cyclopropyl)- 1H-1,2,3-triazol-1-yl)-3,3- dimethylbutanoyl)-4-hydroxy-N-((S)- 1-(4-(4-methylthiazol-5- yl)phenyl)ethyl)pyrrolidine-2- carboxamide |
| A65 | 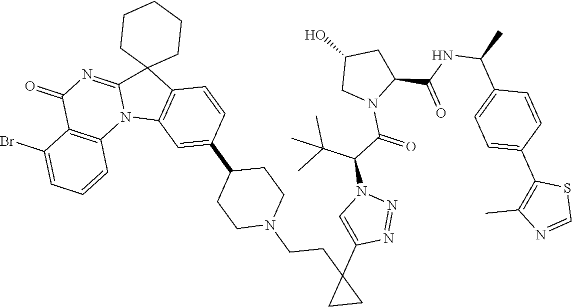 | (2S,4R)-1-((S)-2-(4-(1-(2-(4-(4′- bromo-5′-oxo-5′H-spiro[cyclohexane- 1,7′-indolo[1,2-a]quinazolin]-10′- yl)piperidin-1-yl)ethyl)cyclopropyl)- 1H-1,2,3-triazol-1-yl)-3,3- dimethylbutanoyl)-4-hydroxy-N-((S)- 1-(4-(4-methylthiazol-5- yl)phenyl)ethyl)pyrrolidine-2- carboxamide |
| A66 | 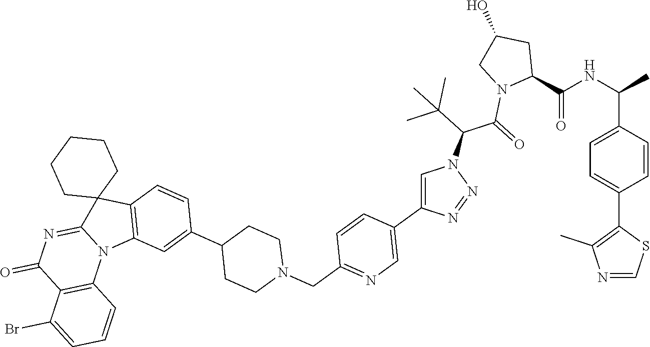 [SMILES: Cc1ncsc1-c1ccc([C@H](C)NC(=O)[C@@H]2C[C@@H](O)CN2C(=O)[C@@H](n2cc(-c3ccc(CN4CCC(c5ccc6c(c5)-n5c(nc(=O)c7c(Br)cccc75)C65CCCCC5)CC4)nc3)nn2)C(C)(C)C)cc1] | (2S,4R)-1-((S)-2-(4-(6-((4-(4′-bromo- 5′-oxo-5′H-spiro[cyclohexane-1,7′- indolo[1,2-a]quinazolin]-10′- yl)piperidin-1-yl)methyl)pyridin-3-yl)- 1H-1,2,3-triazol-1-yl)-3,3- dimethylbutanoyl)-4-hydroxy-N-((S)- 1-(4-(4-methylthiazol-5- yl)phenyl)ethyl)pyrrolidine-2- carboxamide |
| A67 | 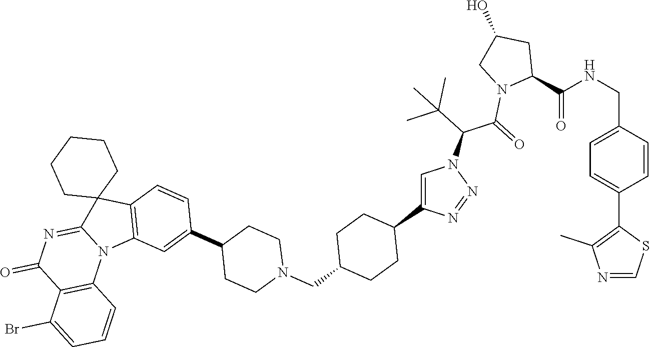 | (2S,4R)-1-((S)-2-(4-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-(4-(4- methylthiazol-5-yl)benzyl)pyrrolidine- 2-carboxamide |
| A68 | 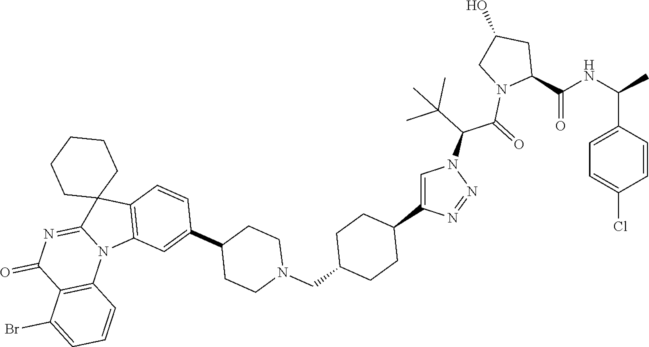 | (2S,4R)-1-((S)-2-(4-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-N-((S)-1-(4- chlorophenyl)ethyl)-4- hydroxypyrrolidine-2-carboxamide |
| A69 | 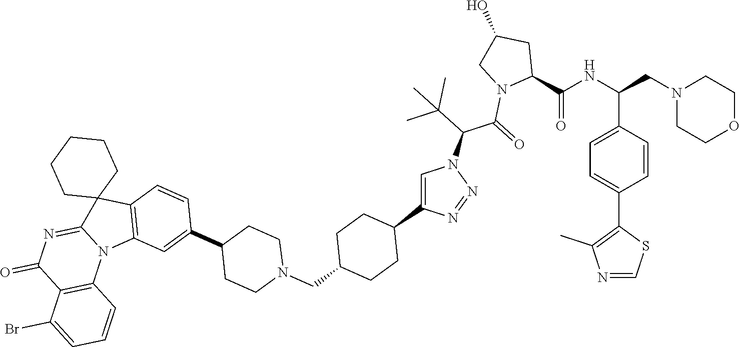 | (2S,4R)-1-((S)-2-(4-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 1-(4-(4-methylthiazol-5-yl)phenyl)-2- morpholinoethyl)pyrrolidine-2- carboxamide |
| A70 | 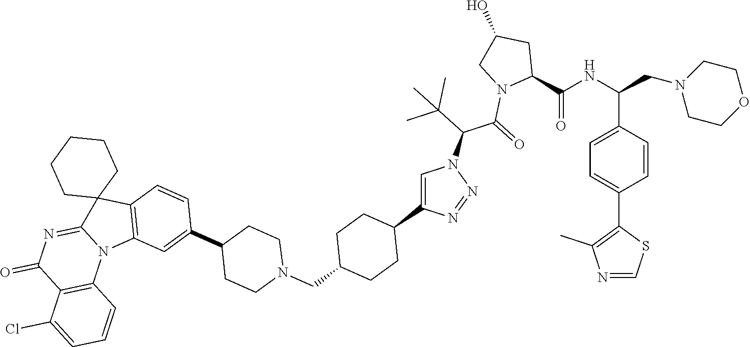 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 1-(4-(4-methylthiazol-5-yl)phenyl)-2- morpholinoethyl)pyrrolidine-2- carboxamide |
| A71 | 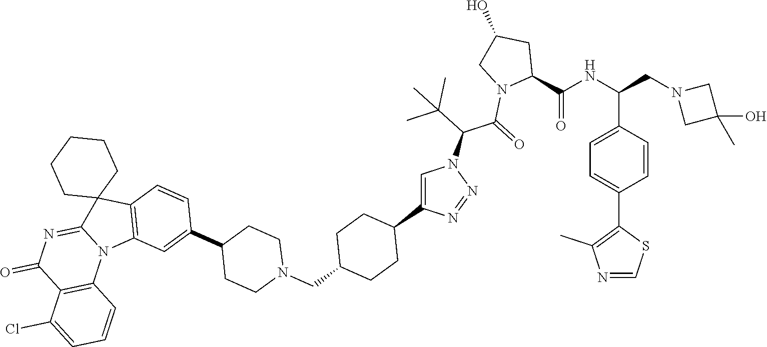 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 2-(3-hydroxy-3-methylazetidin-1-yl)-1- (4-(4-methylthiazol-5- yl)phenyl)ethyl)pyrrolidine-2- carboxamide |
| A72 | 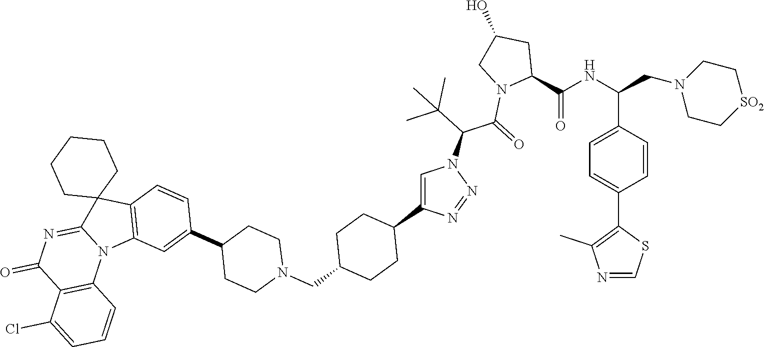 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-N-((R)-2-(1,1- dioxidothiomorpholino)-1-(4-(4- methylthiazol-5-yl)phenyl)ethyl)-4- hydroxypyrrolidine-2-carboxamide |
| A73 | 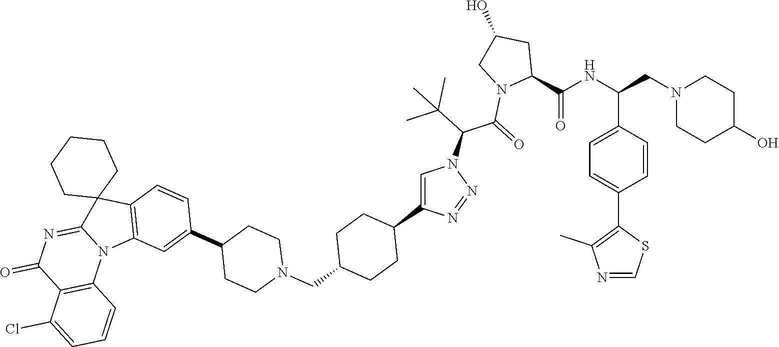 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 2-(4-hydroxypiperidin-1-yl)-1-(4-(4- methylthiazol-5- yl)phenyl)ethyl)pyrrolidine-2- carboxamide |
| A74 | 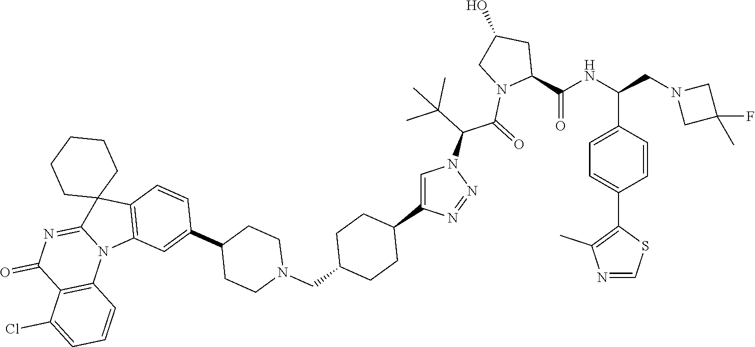 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-N-((R)-2-(3-fluoro- 3-methylazetidin-1-yl)-1-(4-(4- methylthiazol-5-yl)phenyl)ethyl)-4- hydroxypyrrolidine-2-carboxamide |
| A75 | 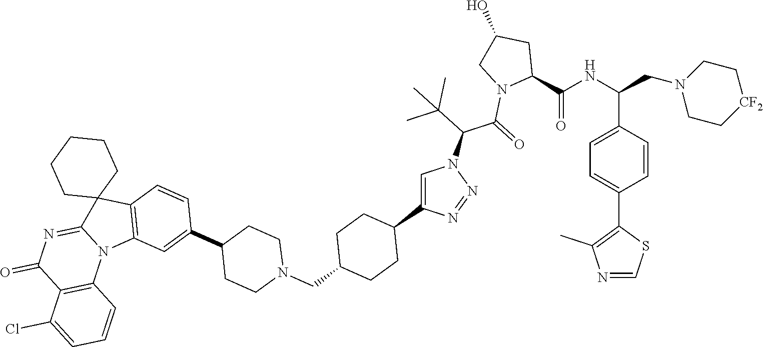 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-N-((R)-2-(4,4- difluoropiperidin-1-yl)-1-(4-(4- methylthiazol-5-yl)phenyl)ethyl)-4- hydroxypyrrolidine-2-carboxamide |
| A76 | 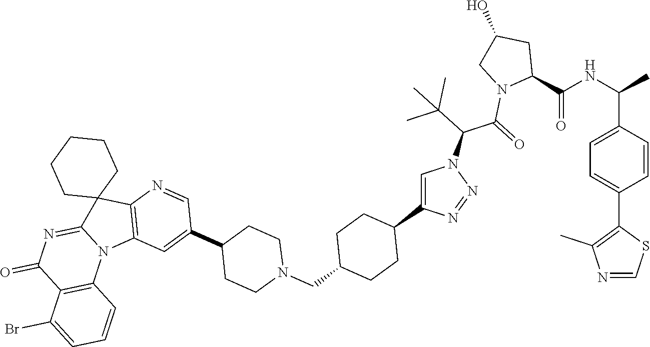 | (2S,4R)-1-((S)-2-(4-((1r,4S)-4-((4-(4′- bromo-5′-oxo-5′H-spiro[cyclohexane- 1,7′-pyrido[2′,3′:4,5]pyrrolo[1,2- a]quinazolin]-10′-yl)piperidin-1- yl)methyl)cyclohexyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A77 | 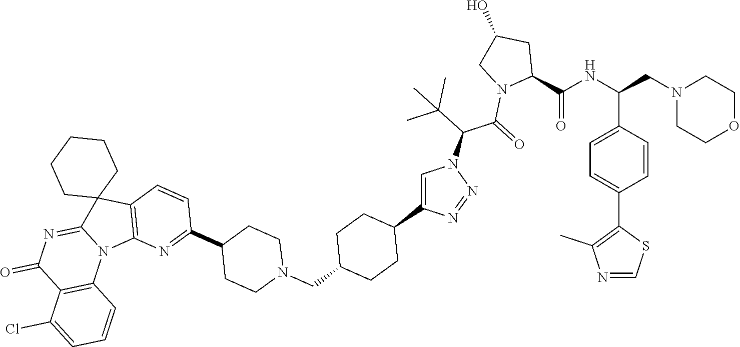 | (2S,4R)-1-((S)-2-(4-((1r,4S)-4-((4-(4′- chloro-5′-oxo-5′H-spiro[cyclohexane- 1,7′-pyrido[3′,2′:4,5]pyrrolo[1,2- a]quinazolin]-10′-yl)piperidin-1- yl)methyl)cyclohexyl)-1H-1,2,3- triazol-1-yl)-3,3-dimethylbutanoyl)-4- hydroxy-N-((R)-1-(4-(4-methylthiazol- 5-yl)phenyl)-2- morpholinoethyl)pyrrolidine-2- carboxamide |
| A78 | 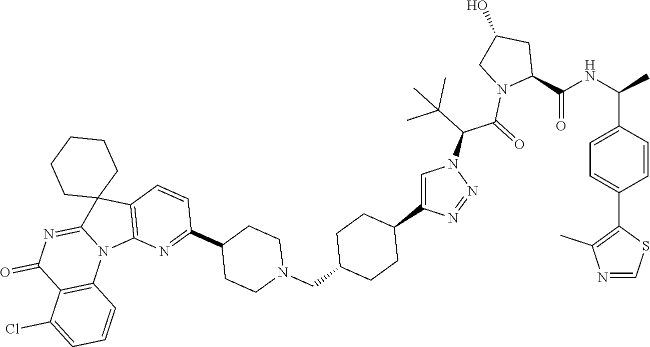 | (2S,4R)-1-((S)-2-(4-((1r,4S)-4-((4-(4′- chloro-5′-oxo-5′H-spiro[cyclohexane- 1,7′-pyrido[3′,2′:4,5]pyrrolo[1,2- a]quinazolin]-10′-yl)piperidin-1- yl)methyl)cyclohexyl)-1H-1,2,3- triazol-1-yl)-3,3-dimethylbutanoyl)-4- hydroxy-N-((S)-1-(4-(4-methylthiazol- 5-yl)phenyl)ethyl)pyrrolidine-2- carboxamide |
| A79 | 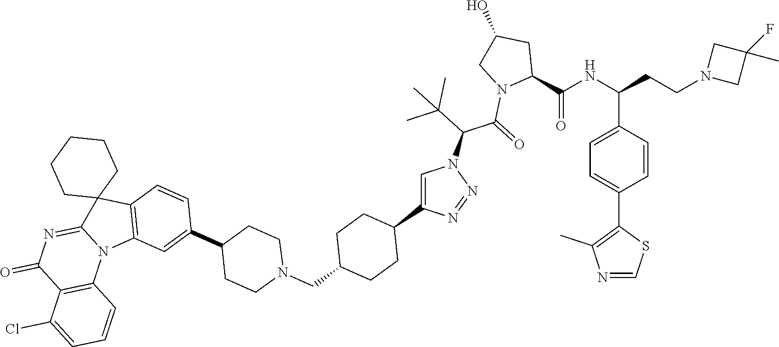 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-N-((S)-3-(3-fluoro- 3-methylazetidin-1-yl)-1-(4-(4- methylthiazol-5-yl)phenyl)propyl)-4- hydroxypyrrolidine-2-carboxamide |
| A80 | 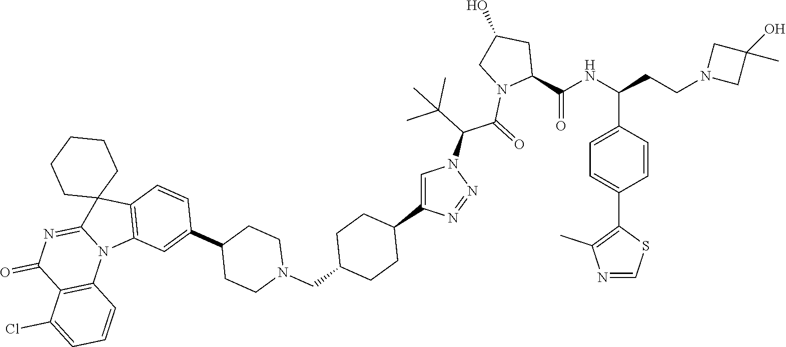 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((S)- 3-(3-hydroxy-3-methylazetidin-1-yl)-1- (4-(4-methylthiazol-5- yl)phenyl)propyl)pyrrolidine-2- carboxamide |
| A81 | 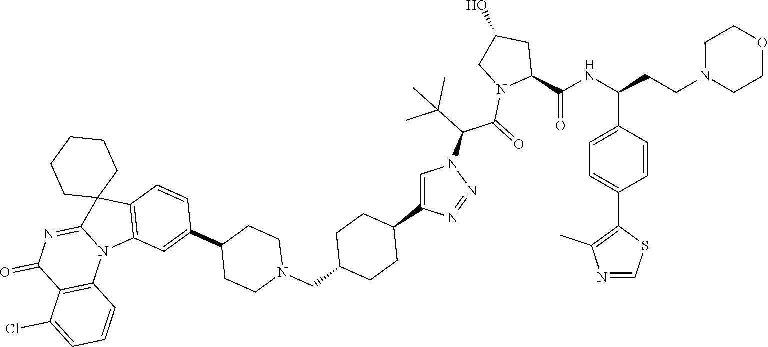 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((S)- 1-(4-(4-methylthiazol-5-yl)phenyl)-3- morpholinopropyl)pyrrolidine-2- carboxamide |
| A82 | 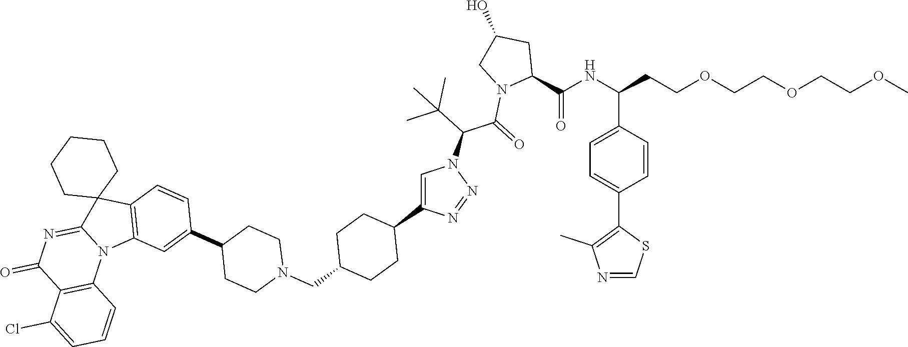 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((S)- 3-(2-(2-methoxyethoxy)ethoxy)-1-(4- (4-methylthiazol-5- yl)phenyl)propyl)pyrrolidine-2- carboxamide |
| A83 | 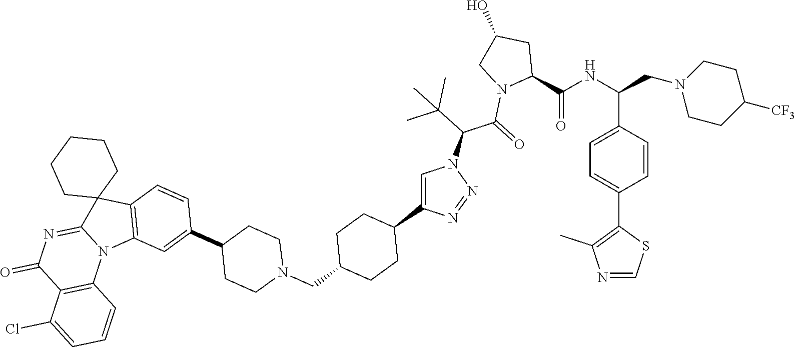 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-(®-1- (4-(4-methylthiazol-5-yl)phenyl)-2-(4- (trifluoromethyl)piperidin-1- yl)ethyl)pyrrolidine-2-carboxamide |
| A84 | 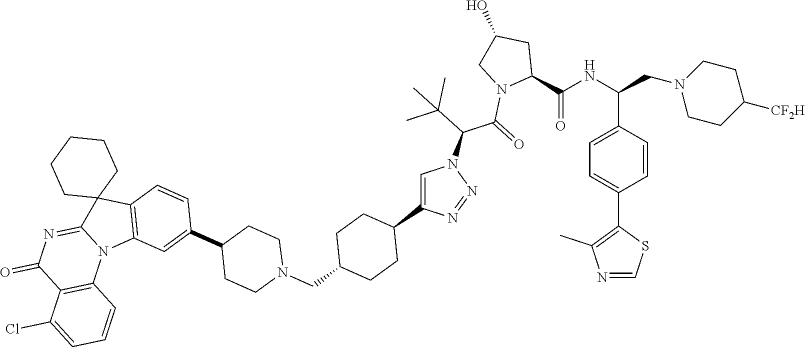 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-N-(®-2-(4- (difluoromethyl)piperidin-1-yl)-1-(4- (4-methylthiazol-5-yl)phenyl)ethyl)-4- hydroxypyrrolidine-2-carboxamide |
| A85 | 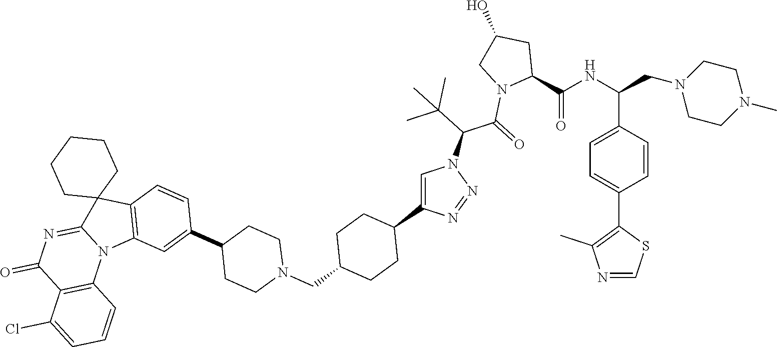 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-(®-2- (4-methylpiperazin-1-yl)-1-(4-(4- methylthiazol-5- yl)phenyl)ethyl)pyrrolidine-2- carboxamide |
| A86 | 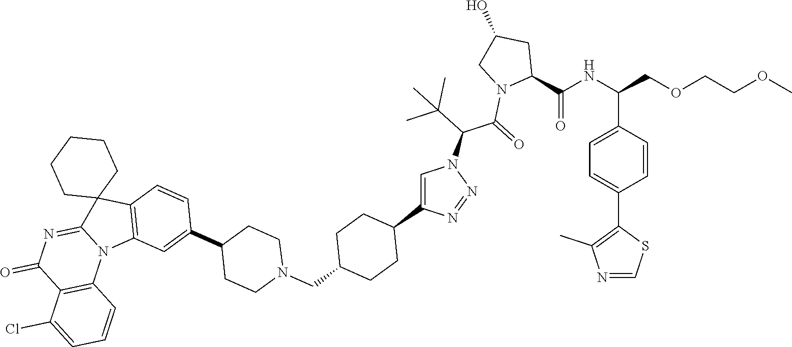 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-(®-2- (2-methoxyethoxy)-1-(4-(4- methylthiazol-5- yl)phenyl)ethyl)pyrrolidine-2- carboxamide |
| A87 | 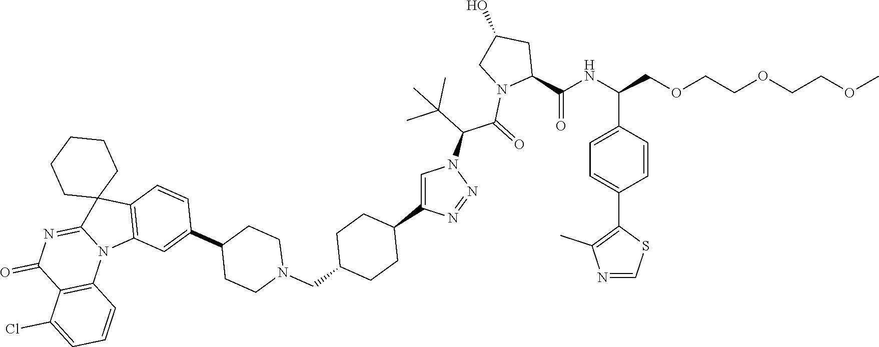 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-(®-2- (2-(2-methoxyethoxy)ethoxy)-1-(4-(4- methylthiazol-5- yl)phenyl)ethyl)pyrrolidine-2- carboxamide |
| A88 | 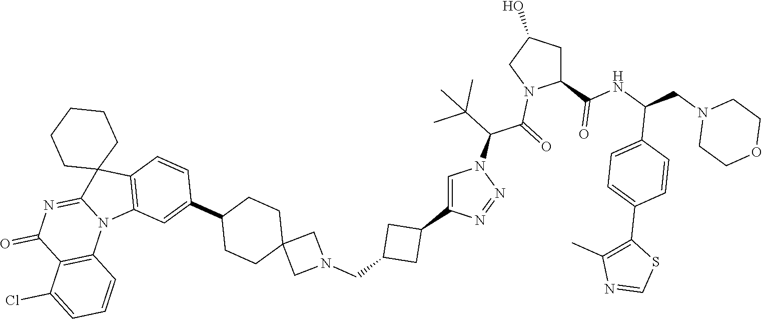 | (2S,4R)-1-((S)-2-(4-((1r,3S)-3-((7-(4′- chloro-5′-oxo-5′H-spiro[cyclohexane- 1,7′-indolo[1,2-a]quinazolin]-10′-yl)- 2-azaspiro[3.5]nonan-2- yl)methyl)cyclobutyl)-1H-1,2,3- triazol-1-yl)-3,3-dimethylbutanoyl)-4- hydroxy-N-(®-1-(4-(4-methylthiazol- 5-yl)phenyl)-2- morpholinoethyl)pyrrolidine-2- carboxamide |
| A89 | 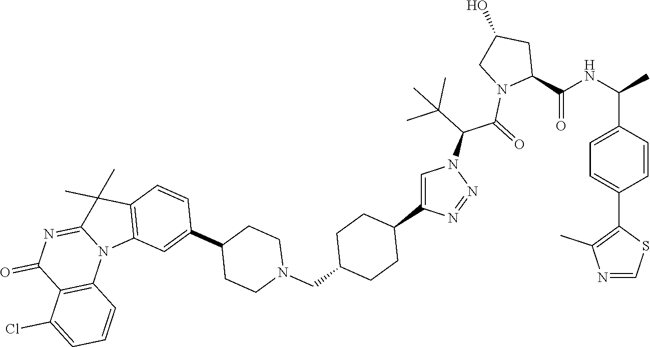 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-3,3- dimethylbutanoyl)-4-hydroxy-N-((S)- 1-(4-(4-methylthiazol-5- yl)phenyl)ethyl)pyrrolidine-2- carboxamide |
| A90 | 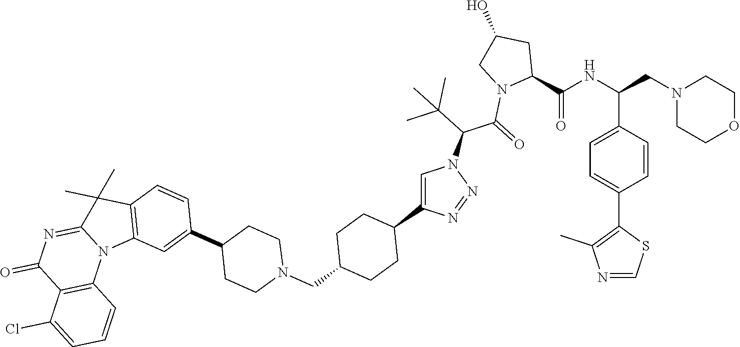 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-3,3- dimethylbutanoyl)-4-hydroxy-N-(®-1- (4-(4-methylthiazol-5-yl)phenyl)-2- morpholinoethyl)pyrrolidine-2- carboxamide |
| A91 | 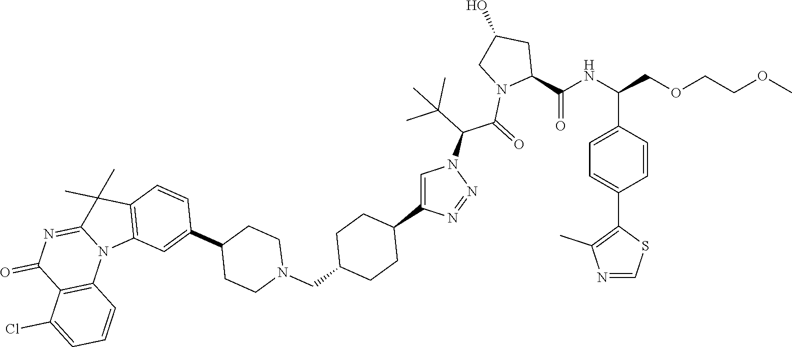 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-3,3- dimethylbutanoyl)-4-hydroxy-N-(®-2- (2-methoxyethoxy)-1-(4-(4- methylthiazol-5- yl)phenyl)ethyl)pyrrolidine-2- carboxamide |
| A92 | 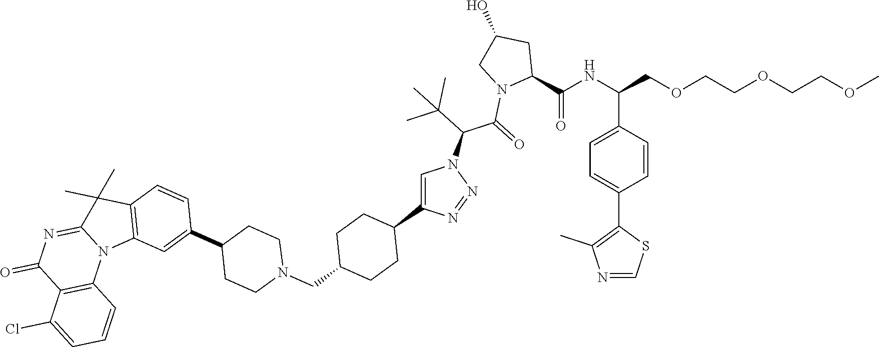 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-3,3- dimethylbutanoyl)-4-hydroxy-N-(®-2- (2-(2-methoxyethoxy)ethoxy)-1-(4-(4- methylthiazol-5- yl)phenyl)ethyl)pyrrolidine-2- carboxamide |
| A93 | 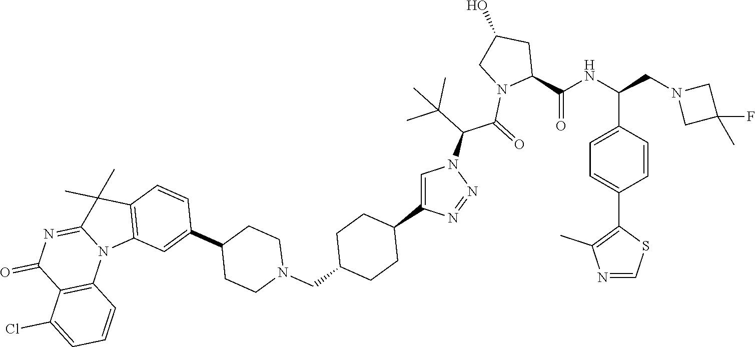 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-3,3- dimethylbutanoyl)-N-(®-2-(3-fluoro-3- methylazetidin-1-yl)-1-(4-(4- methylthiazol-5-yl)phenyl)ethyl)-4- hydroxypyrrolidine-2-carboxamide |
| A94 | 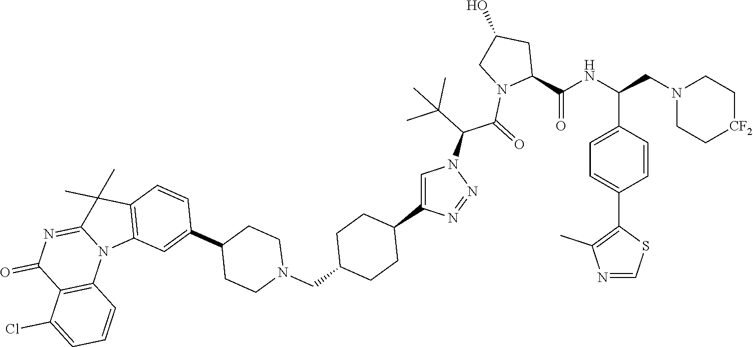 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-3,3- dimethylbutanoyl)-N-(®-2-(4,4- difluoropiperidin-1-yl)-1-(4-(4- methylthiazol-5-yl)phenyl)ethyl)-4- hydroxypyrrolidine-2-carboxamide |
| A95 | 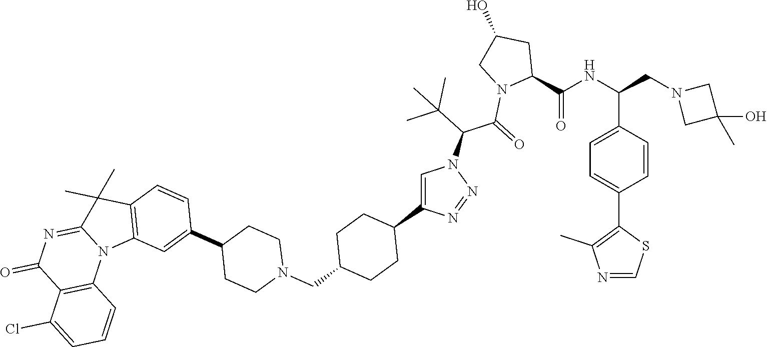 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-3,3- dimethylbutanoyl)-4-hydroxy-N-(®-2- (3-hydroxy-3-methylazetidin-1-yl)-1- (4-(4-methylthiazol-5- yl)phenyl)ethyl)pyrrolidine-2- carboxamide |
| A96 | 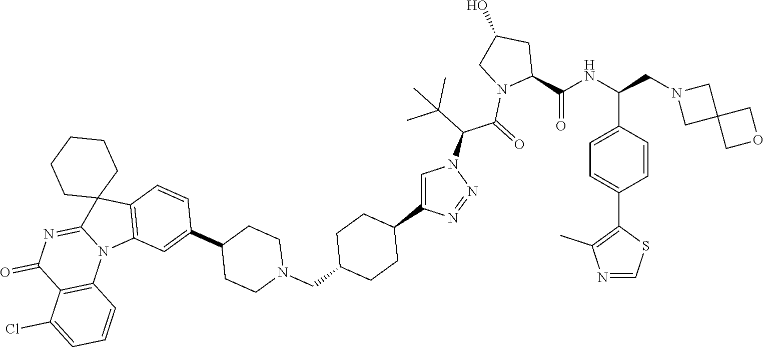 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-(®-1- (4-(4-methylthiazol-5-yl)phenyl)-2-(2- oxa-6-azaspiro[3.3]heptan-6- yl)ethyl)pyrrolidine-2-carboxamide |
| A97 | 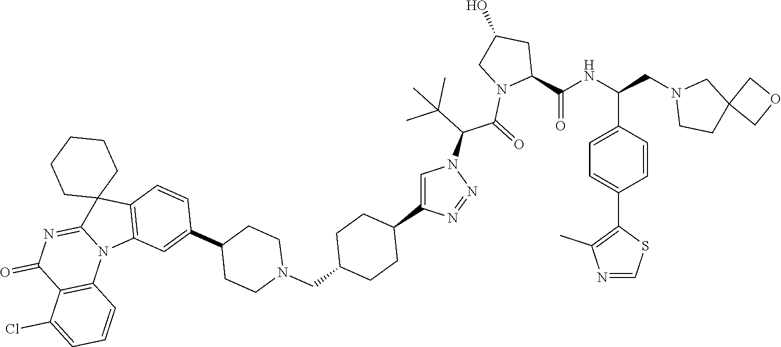 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 1-(4-(4-methylthiazol-5-yl)phenyl)-2- (2-oxa-6-azaspiro[3.4]octan-6- yl)ethyl)pyrrolidine-2-carboxamide |
| A98 | 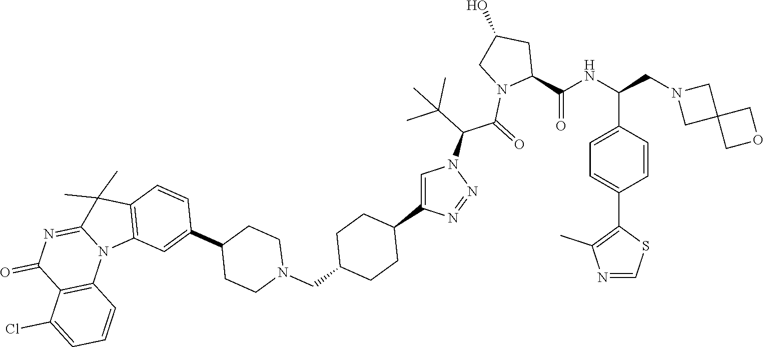 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-3,3- dimethylbutanoyl)-4-hydroxy-N-((R)- 1-(4-(4-methylthiazol-5-yl)phenyl)-2- (2-oxa-6-azaspiro[3.3]heptan-6- yl)ethyl)pyrrolidine-2-carboxamide |
| A99 | 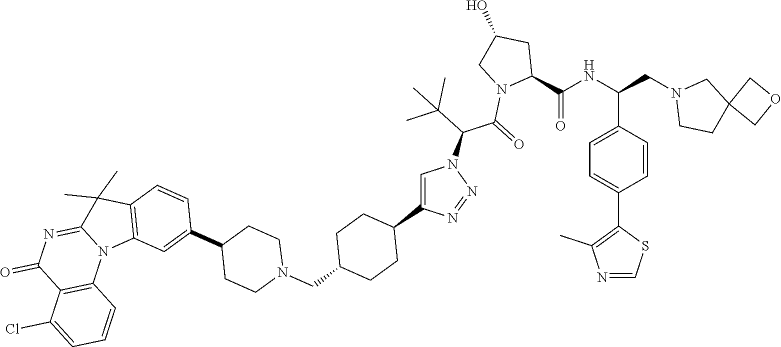 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-3,3- dimethylbutanoyl)-4-hydroxy-N-((R)- 1-(4-(4-methylthiazol-5-yl)phenyl)-2- (2-oxa-6-azaspiro[3.4]octan-6- yl)ethyl)pyrrolidine-2-carboxamide |
| A100 | 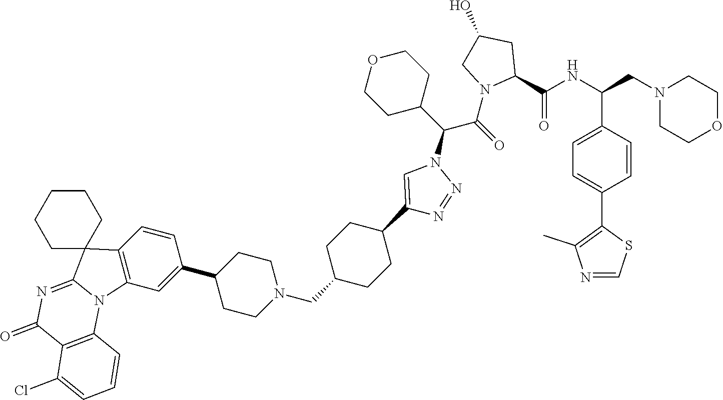 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2-(tetrahydro- 2H-pyran-4-yl)acetyl)-4-hydroxy-N- ((R)-1-(4-(4-methylthiazol-5- yl)phenyl)-2- morpholinoethyl)pyrrolidine-2- carboxamide |
| A101 | 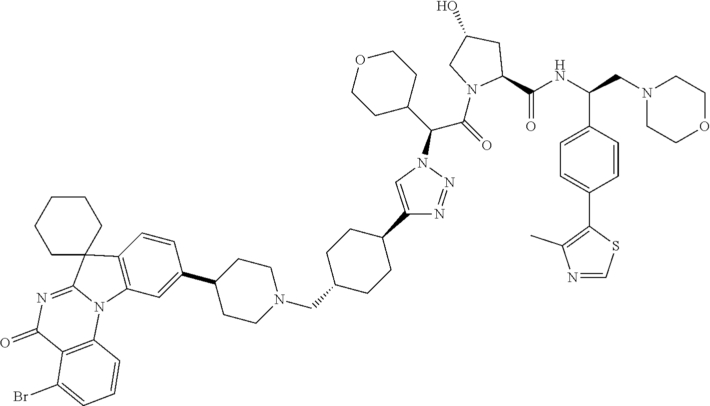 | (2S,4R)-1-((S)-2-(4-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2-(tetrahydro- 2H-pyran-4-yl)acetyl)-4-hydroxy-N- ((R)-1-(4-(4-methylthiazol-5- yl)phenyl)-2- morpholinoethyl)pyrrolidine-2- carboxamide |
| A102 | 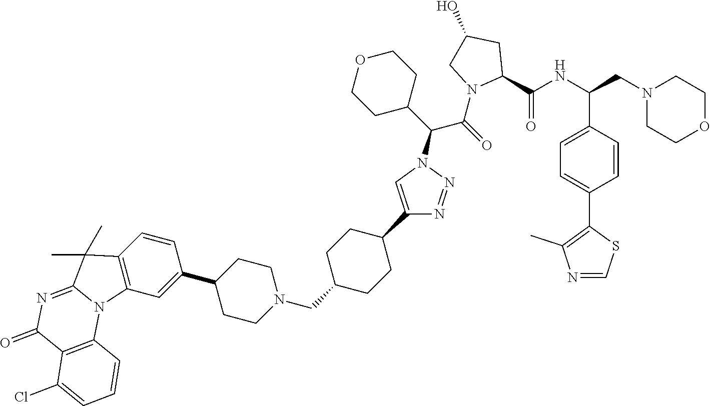 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-2-(tetrahydro- 2H-pyran-4-yl)acetyl)-4-hydroxy-N- ((R)-1-(4-(4-methylthiazol-5- yl)phenyl)-2- morpholinoethyl)pyrrolidine-2- carboxamide |
| A103 | 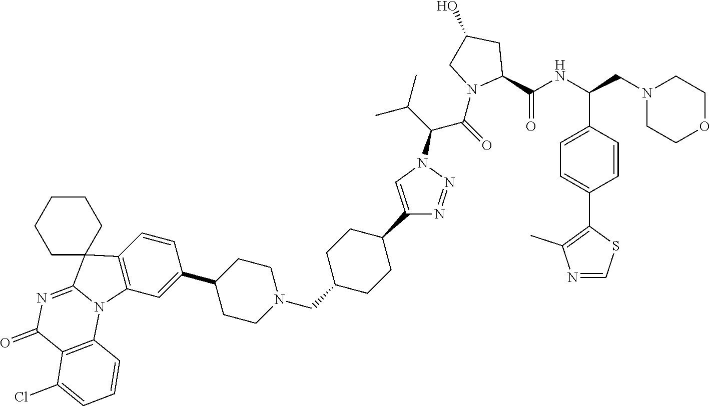 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3- methylbutanoyl)-4-hydroxy-N-((R)-1- (4-(4-methylthiazol-5-yl)phenyl)-2- morpholinoethyl)pyrrolidine-2- carboxamide |
| A104 | 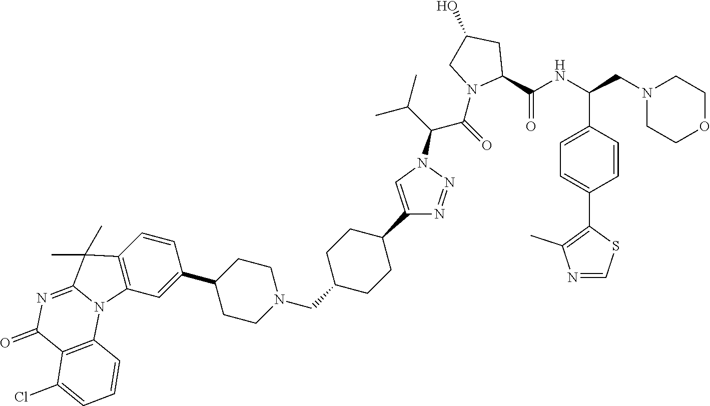 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-3- methylbutanoyl)-4-hydroxy-N-((R)-1- (4-(4-methylthiazol-5-yl)phenyl)-2- morpholinoethyl)pyrrolidine-2- carboxamide |
| A105 | 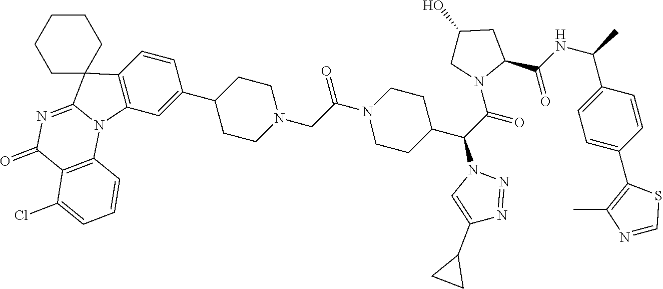 [SMILES: Cc1ncsc1-c1ccc([C@H](C)NC(=O)[C@@H]2C[C@@H](O)CN2C(=O)[C@H](C2CCN(C(=O)CN3CCC(c4ccc5c(c4)-n4c(nc(=O)c6c(Cl)cccc64)C54CCCCC4)CC3)CC2)n2cc(C3CC3)nn2)cc1] | (2S,4R)-1-((S)-2-(1-(2-(4-(4′-chloro-5′- oxo-5′H-spiro[cyclohexane-1,7′- indolo[1,2-a]quinazolin]-10′- yl)piperidin-1-yl)acetyl)piperidin-4- yl)-2-(4-cyclopropyl-1H-1,2,3-triazol- 1-yl)acetyl)-4-hydroxy-N-((S)-1-(4-(4- methylthiazol-5- yl)phenyl)ethyl)pyrrolidine-2- carboxamide |
| A106 | 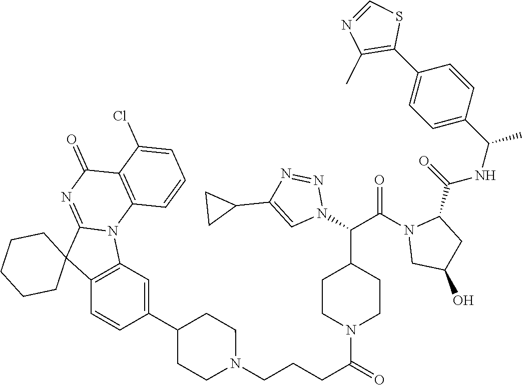 [SMILES: Cc1ncsc1-c1ccc([C@H](C)NC(=O)[C@@H]2C[C@@H](O)CN2C(=O)[C@H](C2CCN(C(=O)CCCN3CCC(c4ccc5c(c4)-n4c(nc(=O)c6c(Cl)cccc64)C54CCCCC4)CC3)CC2)n2cc(C3CC3)nn2)cc1] | (2S,4R)-1-((S)-2-(1-(4-(4-(4′-chloro-5′- oxo-5′H-spiro[cyclohexane-1,7′- indolo[1,2-a]quinazolin]-10′- yl)piperidin-1-yl)butanoyl)piperidin-4- yl)-2-(4-cyclopropyl-1H-1,2,3-triazol- 1-yl)acetyl)-4-hydroxy-N-((S)-1-(4-(4- methylthiazol-5- yl)phenyl)ethyl)pyrrolidine-2- carboxamide |
| A107 | 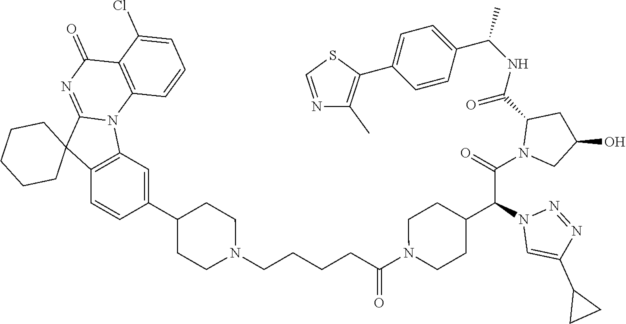 [SMILES: Cc1ncsc1-c1ccc([C@H](C)NC(=O)[C@@H]2C[C@@H](O)CN2C(=O)[C@H](C2CCN(C(=O)CCCCN3CCC(c4ccc5c(c4)-n4c(nc(=O)c6c(Cl)cccc64)C54CCCCC4)CC3)CC2)n2cc(C3CC3)nn2)cc1] | (2S,4R)-1-((S)-2-(1-(5-(4-(4′-chloro-5′- oxo-5′H-spiro[cyclohexane-1,7′- indolo[1,2-a]quinazolin]-10′- yl)piperidin-1-yl)pentanoyl)piperidin- 4-yl)-2-(4-cyclopropyl-1H-1,2,3- triazol-1-yl)acetyl)-4-hydroxy-N-((S)- 1-(4-(4-methylthiazol-5- yl)phenyl)ethyl)pyrrolidine-2- carboxamide |
| A108 | 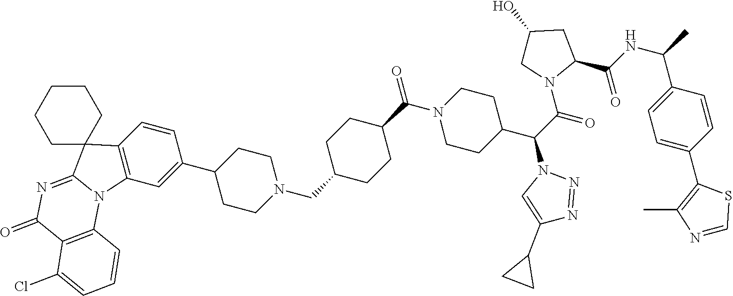 [SMILES: Cc1ncsc1-c1ccc([C@H](C)NC(=O)[C@@H]2C[C@@H](O)CN2C(=O)[C@H](C2CCN(C(=O)[C@H]3CC[C@H](CN4CCC(c5ccc6c(c5)-n5c(nc(=O)c7c(Cl)cccc75)C65CCCCC5)CC4)CC3)CC2)n2cc(C3CC3)nn2)cc1] | (2S,4R)-1-((S)-2-(1-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexane- 1-carbonyl)piperidin-4-yl)-2-(4- cyclopropyl-1H-1,2,3-triazol-1- yl)acetyl)-4-hydroxy-N-((S)-1-(4-(4- methylthiazol-5- yl)phenyl)ethyl)pyrrolidine-2- carboxamide |
| A109 | 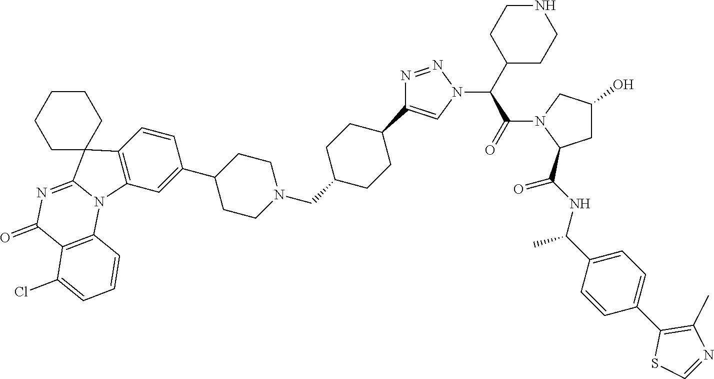 [SMILES: Cc1ncsc1-c1ccc([C@H](C)NC(=O)[C@@H]2C[C@@H](O)CN2C(=O)[C@H](C2CCNCC2)n2cc([C@H]3CC[C@H](CN4CCC(c5ccc6c(c5)-n5c(nc(=O)c7c(Cl)cccc75)C65CCCCC5)CC4)CC3)nn2)cc1] | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2-(piperidin-4- yl)acetyl)-4-hydroxy-N-((S)-1-(4-(4- methylthiazol-5- yl)phenyl)ethyl)pyrrolidine-2- carboxamide |
| A110 | 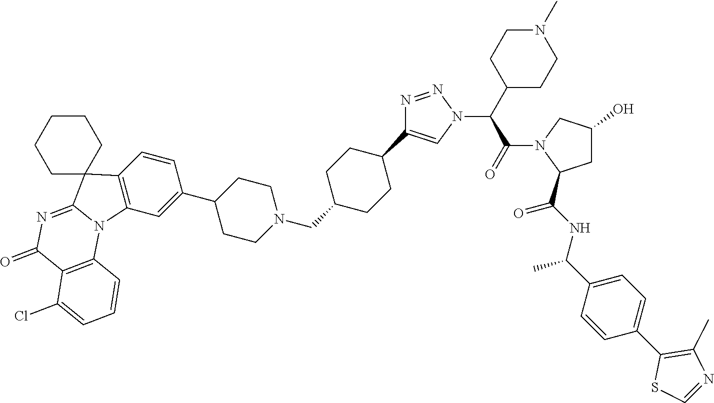 [SMILES: Cc1ncsc1-c1ccc([C@H](C)NC(=O)[C@@H]2C[C@@H](O)CN2C(=O)[C@H](C2CCN(C)CC2)n2cc([C@H]3CC[C@H](CN4CCC(c5ccc6c(c5)-n5c(nc(=O)c7c(Cl)cccc75)C65CCCCC5)CC4)CC3)nn2)cc1] | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2-(1- methylpiperidin-4-yl)acetyl)-4- hydroxy-N-((S)-1-(4-(4-methylthiazol- 5-yl)phenyl)ethyl)pyrrolidine-2- carboxamide |
| A111 | 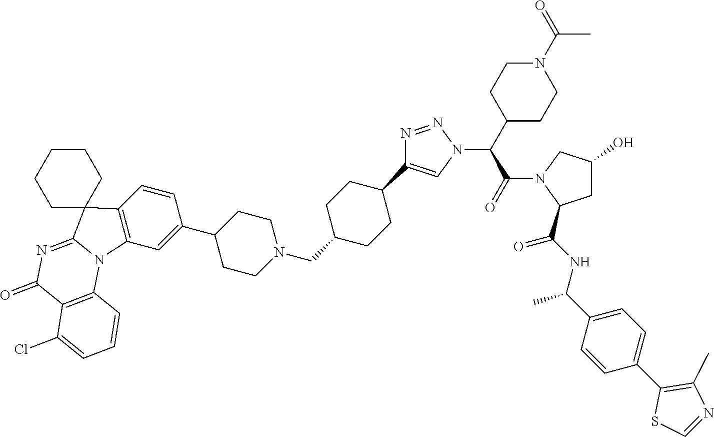 [SMILES: CC(=O)N1CCC([C@@H](C(=O)N2C[C@H](O)C[C@H]2C(=O)N[C@@H](C)c2ccc(-c3scnc3C)cc2)n2cc([C@H]3CC[C@H](CN4CCC(c5ccc6c(c5)-n5c(nc(=O)c7c(Cl)cccc75)C65CCCCC5)CC4)CC3)nn2)CC1] | (2S,4R)-1-((S)-2-(1-acetylpiperidin-4- yl)-2-(4-((1r,4S)-4-((4-(4′-chloro-5′- oxo-5′H-spiro[cyclohexane-1,7′- indolo[1,2-a]quinazolin]-10′- yl)piperidin-1-yl)methyl)cyclohexyl)- 1H-1,2,3-triazol-1-yl)acetyl)-4- hydroxy-N-((S)-1-(4-(4-methylthiazol- 5-yl)phenyl)ethyl)pyrrolidine-2- carboxamide |
| A112 | 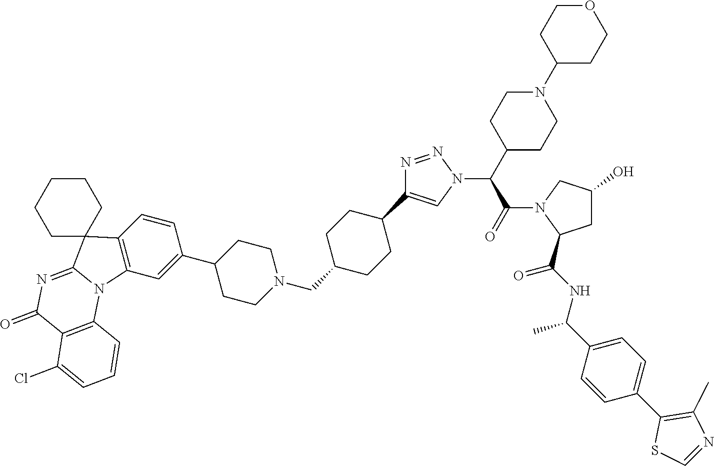 [SMILES: Cc1ncsc1-c1ccc([C@H](C)NC(=O)[C@@H]2C[C@@H](O)CN2C(=O)[C@H](C2CCN(C3CCOCC3)CC2)n2cc([C@H]3CC[C@H](CN4CCC(c5ccc6c(c5)-n5c(nc(=O)c7c(Cl)cccc75)C65CCCCC5)CC4)CC3)nn2)cc1] | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2-(1-(tetrahydro- 2H-pyran-4-yl)piperidin-4-yl)acetyl)-4- hydroxy-N-((S)-1-(4-(4-methylthiazol- 5-yl)phenyl)ethyl)pyrrolidine-2- carboxamide |
| A113 | 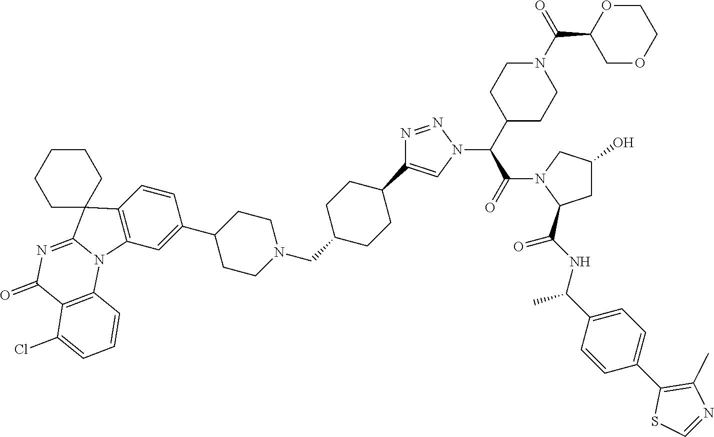 [SMILES: Cc1ncsc1-c1ccc([C@H](C)NC(=O)[C@@H]2C[C@@H](O)CN2C(=O)[C@H](C2CCN(C(=O)[C@@H]3COCCO3)CC2)n2cc([C@H]3CC[C@H](CN4CCC(c5ccc6c(c5)-n5c(nc(=O)c7c(Cl)cccc75)C65CCCCC5)CC4)CC3)nn2)cc1] | (2S,4R)-1-((S)-2-(1-((S)-1,4-dioxane- 2-carbonyl)piperidin-4-yl)-2-(4- ((1r,4S)-4-((4-(4′-chloro-5′-oxo-5′H- spiro[cyclohexane-1,7′-indolo[1,2- a]quinazolin]-10′-yl)piperidin-1- yl)methyl)cyclohexyl)-1H-1,2,3- triazol-1-yl)acetyl)-4-hydroxy-N-((S)- 1-(4-(4-methylthiazol-5- yl)phenyl)ethyl)pyrrolidine-2- carboxamide |
| A114 | 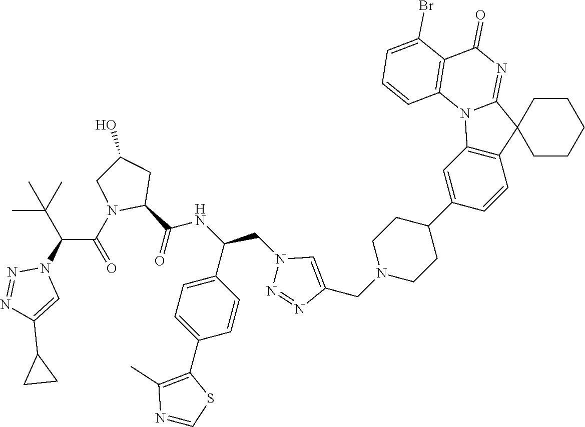 [SMILES: Cc1ncsc1-c1ccc([C@H](Cn2cc(CN3CCC(c4ccc5c(c4)-n4c(nc(=O)c6c(Br)cccc64)C54CCCCC4)CC3)nn2)NC(=O)[C@@H]2C[C@@H](O)CN2C(=O)[C@@H](n2cc(C3CC3)nn2)C(C)(C)C)cc1] | (2S,4R)-N-((R)-2-(4-((4-(4′-bromo-5′- oxo-5′H-spiro[cyclohexane-1,7′- indolo[1,2-a]quinazolin]-10′- yl)piperidin-1-yl)methyl)-1H-1,2,3- triazol-1-yl)-1-(4-(4-methylthiazol-5- yl)phenyl)ethyl)-1-((S)-2-(4- cyclopropyl-1H-1,2,3-triazol-1-yl)-3,3- dimethylbutanoyl)-4- hydroxypyrrolidine-2-carboxamide |
| A115 | 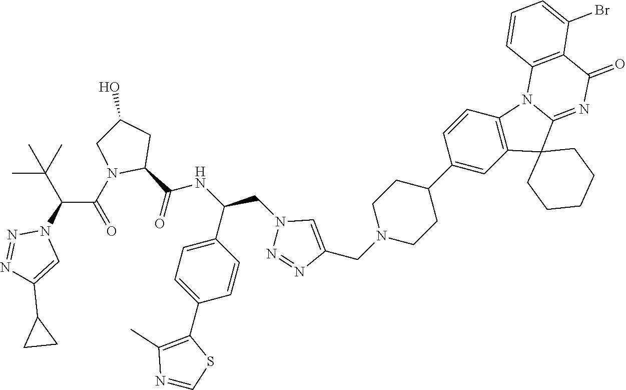 [SMILES: Cc1ncsc1-c1ccc([C@H](Cn2cc(CN3CCC(c4ccc5c(c4)C4(CCCCC4)c4nc(=O)c6c(Br)cccc6n4-5)CC3)nn2)NC(=O)[C@@H]2C[C@@H](O)CN2C(=O)[C@@H](n2cc(C3CC3)nn2)C(C)(C)C)cc1] | (2S,4R)-N-((R)-2-(4-((4-(4′-bromo-5′- oxo-5′H-spiro[cyclohexane-1,7′- indolo[1,2-a]quinazolin]-9′- yl)piperidin-1-yl)methyl)-1H-1,2,3- triazol-1-yl)-1-(4-(4-methylthiazol-5- yl)phenyl)ethyl)-1-((S)-2-(4- cyclopropyl-1H-1,2,3-triazol-1-yl)-3,3- dimethylbutanoyl)-4- hydroxypyrrolidine-2-carboxamide |
| A116 | 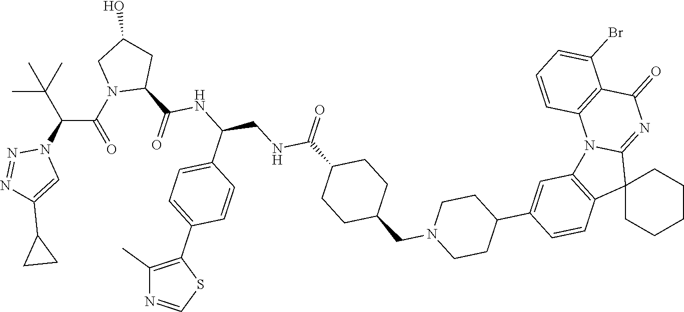 [SMILES: Cc1ncsc1-c1ccc([C@H](CNC(=O)[C@H]2CC[C@H](CN3CCC(c4ccc5c(c4)-n4c(nc(=O)c6c(Br)cccc64)C54CCCCC4)CC3)CC2)NC(=O)[C@@H]2C[C@@H](O)CN2C(=O)[C@@H](n2cc(C3CC3)nn2)C(C)(C)C)cc1] | (2S,4R)-N-((R)-2-((1r,4R)-4-((4-(4′- bromo-5′-oxo-5′H-spiro[cyclohexane- 1,7′-indolo[1,2-a]quinazolin]-10′- yl)piperidin-1-yl)methyl)cyclohexane- 1-carboxamido)-1-(4-(4-methylthiazol- 5-yl)phenyl)ethyl)-1-((S)-2-(4- cyclopropyl-1H-1,2,3-triazol-1-yl)-3,3- dimethylbutanoyl)-4- hydroxypyrrolidine-2-carboxamide |
| A117 | 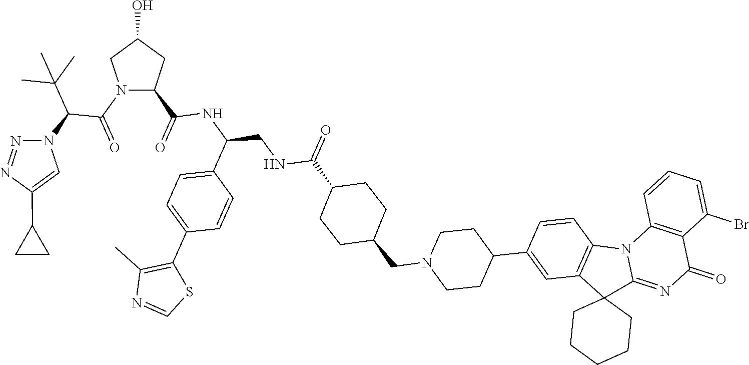 [SMILES: Cc1ncsc1-c1ccc([C@H](CNC(=O)[C@H]2CC[C@H](CN3CCC(c4ccc5c(c4)C4(CCCCC4)c4nc(=O)c6c(Br)cccc6n4-5)CC3)CC2)NC(=O)[C@@H]2C[C@@H](O)CN2C(=O)[C@@H](n2cc(C3CC3)nn2)C(C)(C)C)cc1] | (2S,4R)-N-((R)-2-((1r,4R)-4-((4-(4′- bromo-5′-oxo-5′H-spiro[cyclohexane- 1,7′-indolo[1,2-a]quinazolin]-9′- yl)piperidin-1-yl)methyl)cyclohexane- 1-carboxamido)-1-(4-(4-methylthiazol- 5-yl)phenyl)ethyl)-1-((S)-2-(4- cyclopropyl-1H-1,2,3-triazol-1-yl)-3,3- dimethylbutanoyl)-4- hydroxypyrrolidine-2-carboxamide |
| A118 | 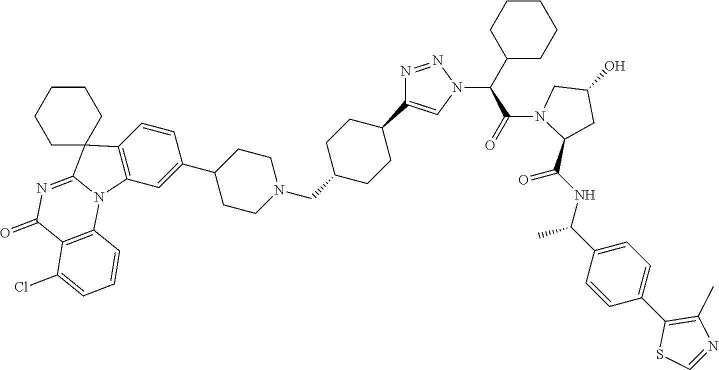 [SMILES: Cc1ncsc1-c1ccc([C@H](C)NC(=O)[C@@H]2C[C@@H](O)CN2C(=O)[C@H](C2CCCCC2)n2cc([C@H]3CC[C@H](CN4CCC(c5ccc6c(c5)-n5c(nc(=O)c7c(Cl)cccc75)C65CCCCC5)CC4)CC3)nn2)cc1] | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2- cyclohexylacetyl)-4-hydroxy-N-((S)-1- (4-(4-methylthiazol-5- yl)phenyl)ethyl)pyrrolidine-2- carboxamide |
| A119 | 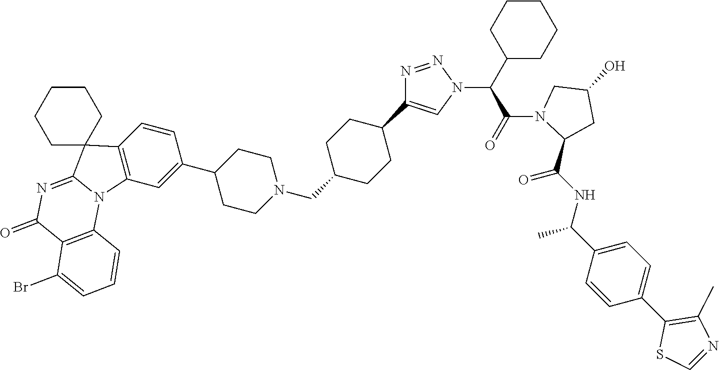 [SMILES: Cc1ncsc1-c1ccc([C@H](C)NC(=O)[C@@H]2C[C@@H](O)CN2C(=O)[C@H](C2CCCCC2)n2cc([C@H]3CC[C@H](CN4CCC(c5ccc6c(c5)-n5c(nc(=O)c7c(Br)cccc75)C65CCCCC5)CC4)CC3)nn2)cc1] | (2S,4R)-1-((S)-2-(4-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2- cyclohexylacetyl)-4-hydroxy-N-((S)-1- (4-(4-methylthiazol-5- yl)phenyl)ethyl)pyrrolidine-2- carboxamide |
| A120 | 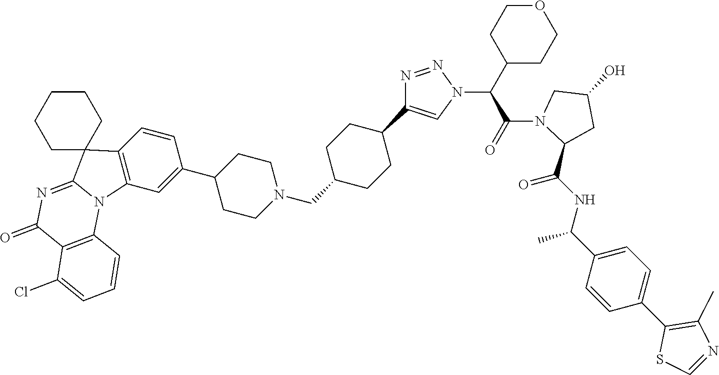 [SMILES: Cc1ncsc1-c1ccc([C@H](C)NC(=O)[C@@H]2C[C@@H](O)CN2C(=O)[C@H](C2CCOCC2)n2cc([C@H]3CC[C@H](CN4CCC(c5ccc6c(c5)-n5c(nc(=O)c7c(Cl)cccc75)C65CCCCC5)CC4)CC3)nn2)cc1] | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2-(tetrahydro- 2H-pyran-4-yl)acetyl)-4-hydroxy-N- ((S)-1-(4-(4-methylthiazol-5- yl)phenyl)ethyl)pyrrolidine-2- carboxamide |
| A121 | 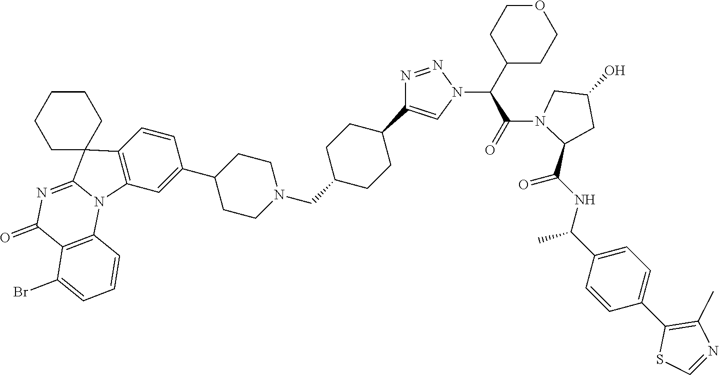 [SMILES: Cc1ncsc1-c1ccc([C@H](C)NC(=O)[C@@H]2C[C@@H](O)CN2C(=O)[C@H](C2CCOCC2)n2cc([C@H]3CC[C@H](CN4CCC(c5ccc6c(c5)-n5c(nc(=O)c7c(Br)cccc75)C65CCCCC5)CC4)CC3)nn2)cc1] | (2S,4R)-1-((S)-2-(4-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2-(tetrahydro- 2H-pyran-4-yl)acetyl)-4-hydroxy-N- ((S)-1-(4-(4-methylthiazol-5- yl)phenyl)ethyl)pyrrolidine-2- carboxamide |
| A122 | 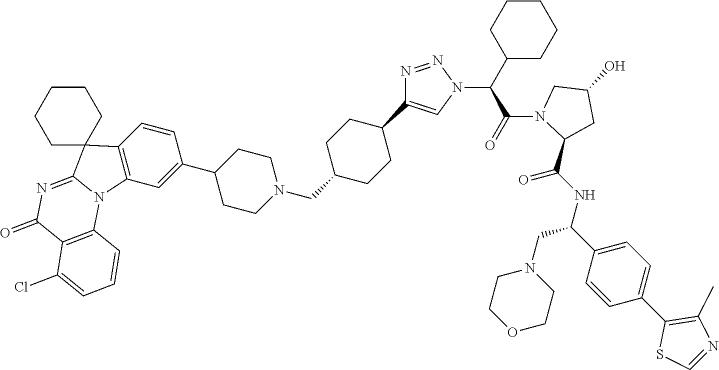 [SMILES: Cc1ncsc1-c1ccc([C@H](CN2CCOCC2)NC(=O)[C@@H]2C[C@@H](O)CN2C(=O)[C@H](C2CCCCC2)n2cc([C@H]3CC[C@H](CN4CCC(c5ccc6c(c5)-n5c(nc(=O)c7c(Cl)cccc75)C65CCCCC5)CC4)CC3)nn2)cc1] | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2- cyclohexylacetyl)-4-hydroxy-N-((R)-1- (4-(4-methylthiazol-5-yl)phenyl)-2- morpholinoethyl)pyrrolidine-2- carboxamide |
| A123 | 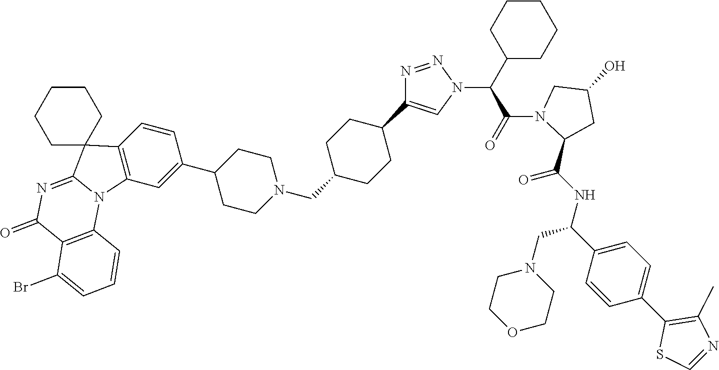 [SMILES: Cc1ncsc1-c1ccc([C@H](CN2CCOCC2)NC(=O)[C@@H]2C[C@@H](O)CN2C(=O)[C@H](C2CCCCC2)n2cc([C@H]3CC[C@H](CN4CCC(c5ccc6c(c5)-n5c(nc(=O)c7c(Br)cccc75)C65CCCCC5)CC4)CC3)nn2)cc1] | (2S,4R)-1-((S)-2-(4-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2- cyclohexylacetyl)-4-hydroxy-N-((R)-1- (4-(4-methylthiazol-5-yl)phenyl)-2- morpholinoethyl)pyrrolidine-2- carboxamide |
| A124 | 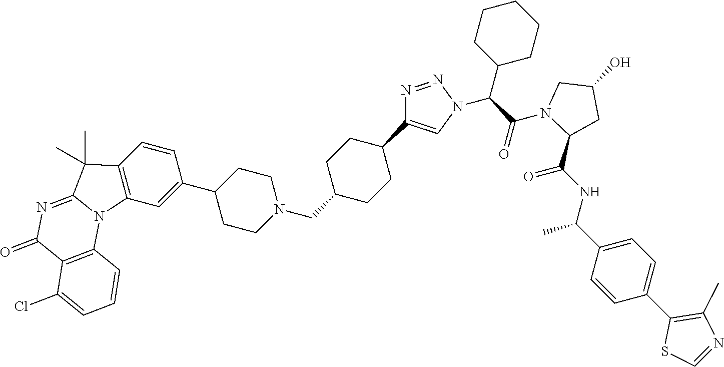 [SMILES: Cc1ncsc1-c1ccc([C@H](C)NC(=O)[C@@H]2C[C@@H](O)CN2C(=O)[C@H](C2CCCCC2)n2cc([C@H]3CC[C@H](CN4CCC(c5ccc6c(c5)-n5c(nc(=O)c7c(Cl)cccc75)C6(C)C)CC4)CC3)nn2)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-2- cyclohexylacetyl)-4-hydroxy-N-((S)-1- (4-(4-methylthiazol-5- yl)phenyl)ethyl)pyrrolidine-2- carboxamide |
| A125 | 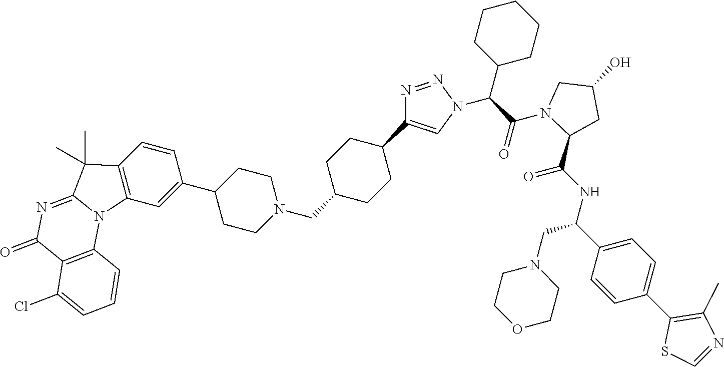 [SMILES: Cc1ncsc1-c1ccc([C@H](CN2CCOCC2)NC(=O)[C@@H]2C[C@@H](O)CN2C(=O)[C@H](C2CCCCC2)n2cc([C@H]3CC[C@H](CN4CCC(c5ccc6c(c5)-n5c(nc(=O)c7c(Cl)cccc75)C6(C)C)CC4)CC3)nn2)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-2- cyclohexylacetyl)-4-hydroxy-N-((R)-1- (4-(4-methylthiazol-5-yl)phenyl)-2- morpholinoethyl)pyrrolidine-2- carboxamide |
| A126 | 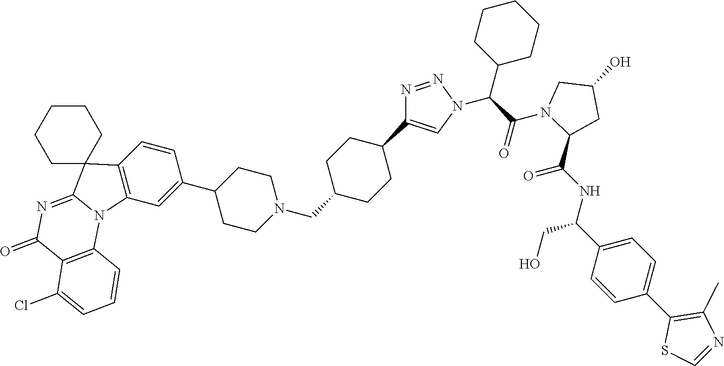 [SMILES: Cc1ncsc1-c1ccc([C@H](CO)NC(=O)[C@@H]2C[C@@H](O)CN2C(=O)[C@H](C2CCCCC2)n2cc([C@H]3CC[C@H](CN4CCC(c5ccc6c(c5)-n5c(nc(=O)c7c(Cl)cccc75)C65CCCCC5)CC4)CC3)nn2)cc1] | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2- cyclohexylacetyl)-4-hydroxy-N-((R)-2- hydroxy-1-(4-(4-methylthiazol-5- yl)phenyl)ethyl)pyrrolidine-2- carboxamide |
| A127 | 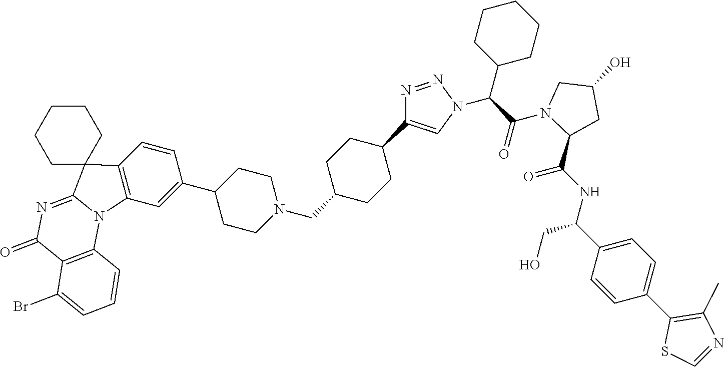 [SMILES: Cc1ncsc1-c1ccc([C@H](CO)NC(=O)[C@@H]2C[C@@H](O)CN2C(=O)[C@H](C2CCCCC2)n2cc([C@H]3CC[C@H](CN4CCC(c5ccc6c(c5)-n5c(nc(=O)c7c(Br)cccc75)C65CCCCC5)CC4)CC3)nn2)cc1] | (2S,4R)-1-((S)-2-(4-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2- cyclohexylacetyl)-4-hydroxy-N-((R)-2- hydroxy-1-(4-(4-methylthiazol-5- yl)phenyl)ethyl)pyrrolidine-2- carboxamide |
| A128 | 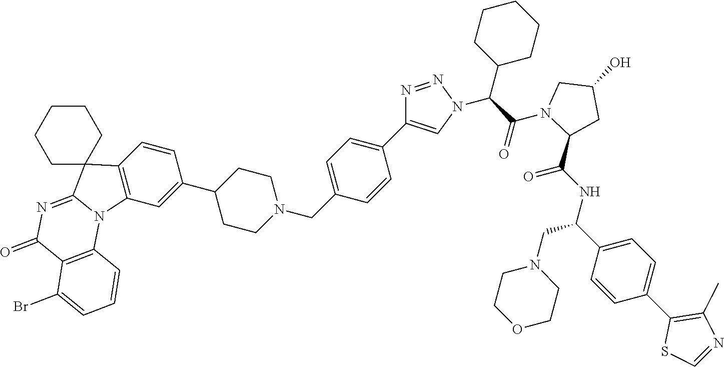 [SMILES: Cc1ncsc1-c1ccc([C@H](CN2CCOCC2)NC(=O)[C@@H]2C[C@@H](O)CN2C(=O)[C@H](C2CCCCC2)n2cc(-c3ccc(CN4CCC(c5ccc6c(c5)-n5c(nc(=O)c7c(Br)cccc75)C65CCCCC5)CC4)cc3)nn2)cc1] | (2S,4R)-1-((S)-2-(4-(4-((4-(4′-bromo- 5′-oxo-5′H-spiro[cyclohexane-1,7′- indolo[1,2-a]quinazolin]-10′- yl)piperidin-1-yl)methyl)phenyl)-1H- 1,2,3-triazol-1-yl)-2-cyclohexylacetyl)- 4-hydroxy-N-((R)-1-(4-(4- methylthiazol-5-yl)phenyl)-2- morpholinoethyl)pyrrolidine-2- carboxamide |
| A129 | 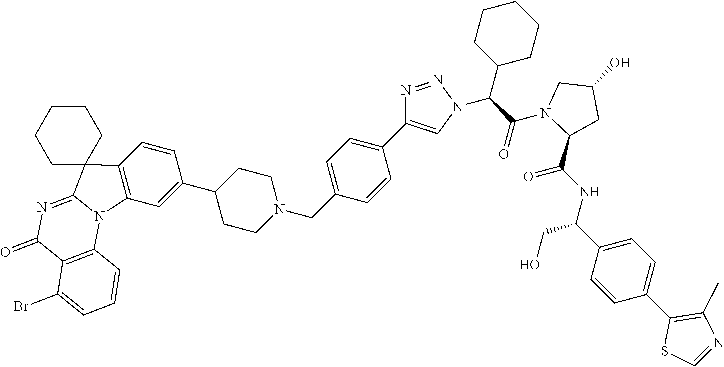 [SMILES: Cc1ncsc1-c1ccc([C@H](CO)NC(=O)[C@@H]2C[C@@H](O)CN2C(=O)[C@H](C2CCCCC2)n2cc(-c3ccc(CN4CCC(c5ccc6c(c5)-n5c(nc(=O)c7c(Br)cccc75)C65CCCCC5)CC4)cc3)nn2)cc1] | (2S,4R)-1-((S)-2-(4-(4-((4-(4′-bromo- 5′-oxo-5′H-spiro[cyclohexane-1,7′- indolo[1,2-a]quinazolin]-10′- yl)piperidin-1-yl)methyl)phenyl)-1H- 1,2,3-triazol-1-yl)-2-cyclohexylacetyl)- 4-hydroxy-N-((R)-2-hydroxy-1-(4-(4- methylthiazol-5- yl)phenyl)ethyl)pyrrolidine-2- carboxamide |
| A130 | 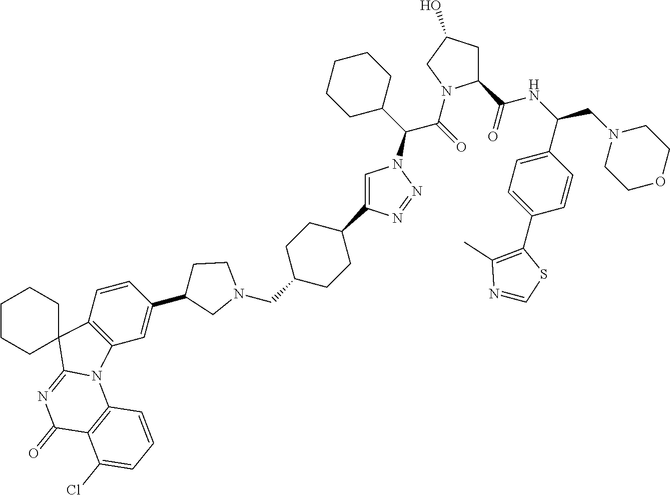 | (2S,4R)-1-((2S)-2-(4-((1r,4S)-4-((3-(4′- chloro-5′-oxo-5′H-spiro[cyclohexane- 1,7′-indolo[1,2-a]quinazolin]-10′- yl)pyrrolidin-1-yl)methyl)cyclohexyl)- 1H-1,2,3-triazol-1-yl)-2- cyclohexylacetyl)-4-hydroxy-N-((R)-1- (4-(4-methylthiazol-5-yl)phenyl)-2- morpholinoethyl)pyrrolidine-2- carboxamide |
| A131 | 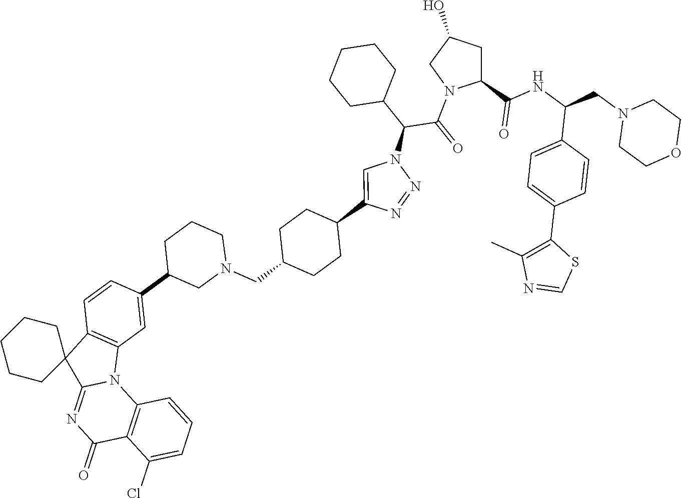 [SMILES: Cc1ncsc1-c1ccc([C@H](CN2CCOCC2)NC(=O)[C@@H]2C[C@@H](O)CN2C(=O)[C@H](C2CCCCC2)n2cc([C@H]3CC[C@H](CN4CCC[C@H](c5ccc6c(c5)-n5c(nc(=O)c7c(Cl)cccc75)C65CCCCC5)C4)CC3)nn2)cc1] | (2S,4R)-1-((2S)-2-(4-((1r,4S)-4-((3-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-2- cyclohexylacetyl)-4-hydroxy-N-((R)-1- (4-(4-methylthiazol-5-yl)phenyl)-2- morpholinoethyl)pyrrolidine-2- carboxamide |
| A132 | 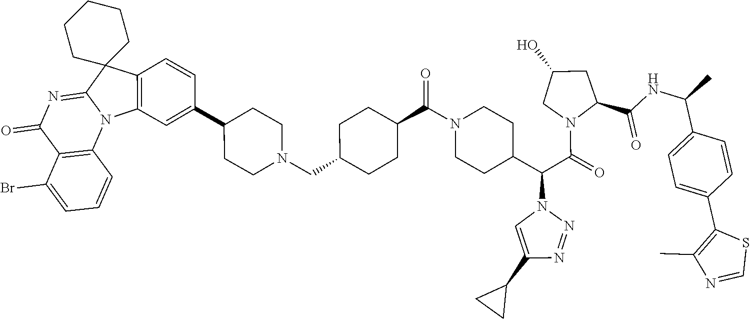 | (2S,4R)-1-((S)-2-(1-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexane- 1-carbonyl)piperidin-4-yl)-2-(4- cyclopropyl-1H-1,2,3-triazol-1- yl)acetyl)-4-hydroxy-N-((S)-1-(4-(4- methylthiazol-5- yl)phenyl)ethyl)pyrrolidine-2- carboxamide |
| A133 | 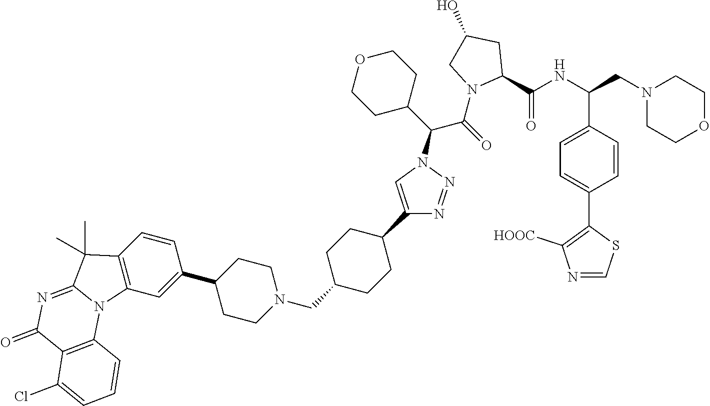 | 5-(4-((R)-1-((2S,4R)-1-((S)-2-(4- ((1r,4S)-4-((4-(4-chloro-7,7-dimethyl- 5-oxo-5,7-dihydroindolo[1,2- a]quinazolin-10-yl)piperidin-1- yl)methyl)cyclohexyl)-1H-1,2,3- triazol-1-yl)-2-(tetrahydro-2H-pyran-4- yl)acetyl)-4-hydroxypyrrolidine-2- carboxamido)-2- morpholinoethyl)phenyl)thiazole-4- carboxylic acid |
| A134 | 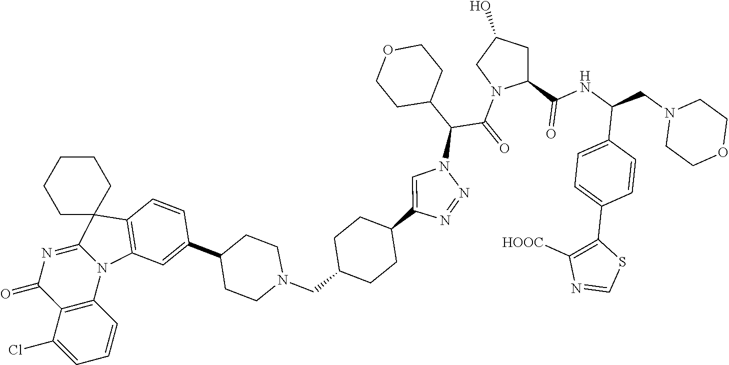 | 5-(4-((R)-1-((2S,4R)-1-((S)-2-(4- ((1r,4S)-4-((4-(4′-chloro-5′-oxo-5′H- spiro[cyclohexane-1,7′-indolo[1,2- a]quinazolin]-10′-yl)piperidin-1- yl)methyl)cyclohexyl)-1H-1,2,3- triazol-1-yl)-2-(tetrahydro-2H-pyran-4- yl)acetyl)-4-hydroxypyrrolidine-2- carboxamido)-2- morpholinoethyl)phenyl)thiazole-4- carboxylic acid |
| A135 | 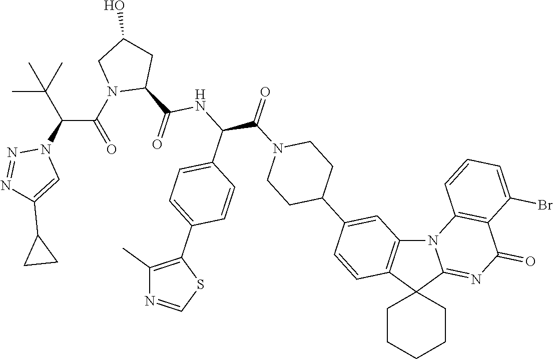 [SMILES: Cc1ncsc1-c1ccc([C@@H](NC(=O)[C@@H]2C[C@@H](O)CN2C(=O)[C@@H](n2cc(C3CC3)nn2)C(C)(C)C)C(=O)N2CCC(c3ccc4c(c3)-n3c(nc(=O)c5c(Br)cccc53)C43CCCCC3)CC2)cc1] | (2S,4R)-N-((R)-2-(4-(4′-bromo-5′-oxo- 5′H-spiro[cyclohexane-1,7′-indolo[1,2- a]quinazolin]-10′-yl)piperidin-1-yl)-1- (4-(4-methylthiazol-5-yl)phenyl)-2- oxoethyl)-1-((S)-2-(4-cyclopropyl-1H- 1,2,3-triazol-1-yl)-3,3- dimethylbutanoyl)-4- hydroxypyrrolidine-2-carboxamide |
| A136 | 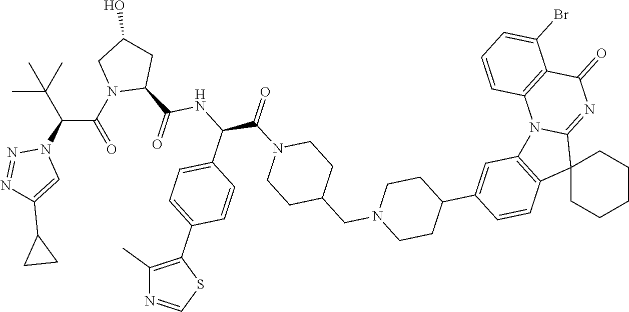 [SMILES: Cc1ncsc1-c1ccc([C@@H](NC(=O)[C@@H]2C[C@@H](O)CN2C(=O)[C@@H](n2cc(C3CC3)nn2)C(C)(C)C)C(=O)N2CCC(CN3CCC(c4ccc5c(c4)-n4c(nc(=O)c6c(Br)cccc64)C54CCCCC4)CC3)CC2)cc1] | (2S,4R)-N-((R)-2-(4-((4-(4′-bromo-5′- oxo-5′H-spiro[cyclohexane-1,7′- indolo[1,2-a]quinazolin]-10′- yl)piperidin-1-yl)methyl)piperidin-1- yl)-1-(4-(4-methylthiazol-5-yl)phenyl)- 2-oxoethyl)-1-((S)-2-(4-cyclopropyl- 1H-1,2,3-triazol-1-yl)-3,3- dimethylbutanoyl)-4- hydroxypyrrolidine-2-carboxamide |
| A137 | 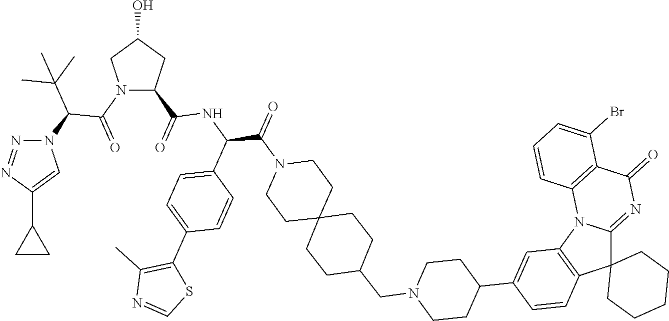 [SMILES: Cc1ncsc1-c1ccc([C@@H](NC(=O)[C@@H]2C[C@@H](O)CN2C(=O)[C@@H](n2cc(C3CC3)nn2)C(C)(C)C)C(=O)N2CCC3(CCC(CN4CCC(c5ccc6c(c5)-n5c(nc(=O)c7c(Br)cccc75)C65CCCCC5)CC4)CC3)CC2)cc1] | (2S,4R)-N-((R)-2-(9-((4-(4′-bromo-5′- oxo-5′H-spiro[cyclohexane-1,7′- indolo[1,2-a]quinazolin]-10′- yl)piperidin-1-yl)methyl)-3- azaspiro[5.5]undecan-3-yl)-1-(4-(4- methylthiazol-5-yl)phenyl)-2- oxoethyl)-1-((S)-2-(4-cyclopropyl-1H- 1,2,3-triazol-1-yl)-3,3- dimethylbutanoyl)-4- hydroxypyrrolidine-2-carboxamide |
| A138 | 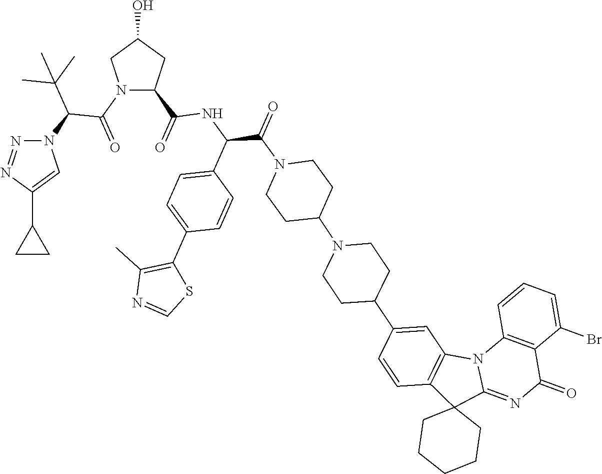 [SMILES: Cc1ncsc1-c1ccc([C@@H](NC(=O)[C@@H]2C[C@@H](O)CN2C(=O)[C@@H](n2cc(C3CC3)nn2)C(C)(C)C)C(=O)N2CCC(N3CCC(c4ccc5c(c4)-n4c(nc(=O)c6c(Br)cccc64)C54CCCCC4)CC3)CC2)cc1] | (2S,4R)-N-((R)-2-(4-(4′-bromo-5′-oxo- 5′H-spiro[cyclohexane-1,7′-indolo[1,2- a]quinazolin]-10′-yl)-[1,4′-bipiperidin]- 1′-yl)-1-(4-(4-methylthiazol-5- yl)phenyl)-2-oxoethyl)-1-((S)-2-(4- cyclopropyl-1H-1,2,3-triazol-1-yl)-3,3- dimethylbutanoyl)-4- hydroxypyrrolidine-2-carboxamide |
| A139 | 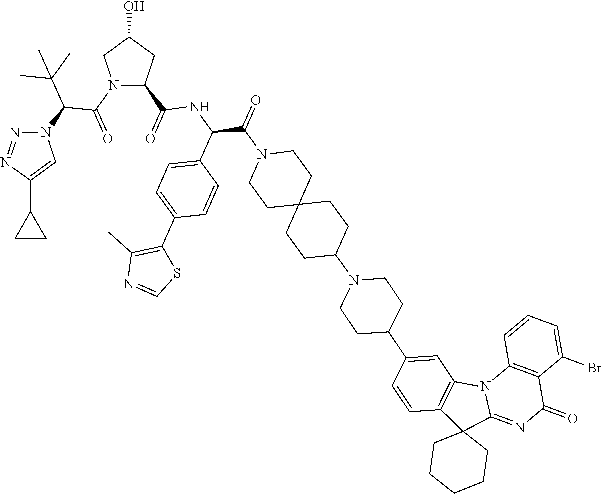 [SMILES: Cc1ncsc1-c1ccc([C@@H](NC(=O)[C@@H]2C[C@@H](O)CN2C(=O)[C@@H](n2cc(C3CC3)nn2)C(C)(C)C)C(=O)N2CCC3(CCC(N4CCC(c5ccc6c(c5)-n5c(nc(=O)c7c(Br)cccc75)C65CCCCC5)CC4)CC3)CC2)cc1] | (2S,4R)-N-((R)-2-(9-(4-(4′-bromo-5′- oxo-5′H-spiro[cyclohexane-1,7′- indolo[1,2-a]quinazolin]-10′- yl)piperidin-1-yl)-3- azaspiro[5.5]undecan-3-yl)-1-(4-(4- methylthiazol-5-yl)phenyl)-2- oxoethyl)-1-((S)-2-(4-cyclopropyl-1H- 1,2,3-triazol-1-yl)-3,3- dimethylbutanoyl)-4- hydroxypyrrolidine-2-carboxamide |
| A140 | 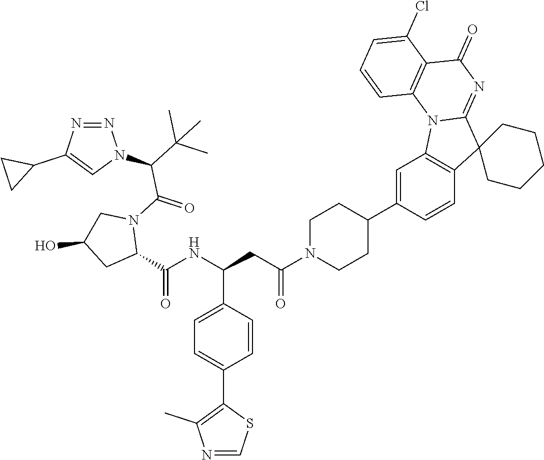 [SMILES: Cc1ncsc1-c1ccc([C@H](CC(=O)N2CCC(c3ccc4c(c3)-n3c(nc(=O)c5c(Cl)cccc53)C43CCCCC3)CC2)NC(=O)[C@@H]2C[C@@H](O)CN2C(=O)[C@@H](n2cc(C3CC3)nn2)C(C)(C)C)cc1] | (2S,4R)-N-((S)-3-(4-(4′-chloro-5′-oxo- 5′H-spiro[cyclohexane-1,7′-indolo[1,2- a]quinazolin]-10′-yl)piperidin-1-yl)-1- (4-(4-methylthiazol-5-yl)phenyl)-3- oxopropyl)-1-((S)-2-(4-cyclopropyl- 1H-1,2,3-triazol-1-yl)-3,3- dimethylbutanoyl)-4- hydroxypyrrolidine-2-carboxamide |
| A141 | 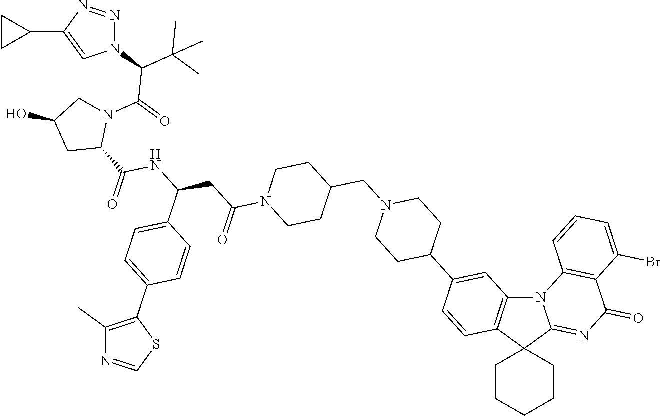 [SMILES: Cc1ncsc1-c1ccc([C@H](CC(=O)N2CCC(CN3CCC(c4ccc5c(c4)-n4c(nc(=O)c6c(Br)cccc64)C54CCCCC4)CC3)CC2)NC(=O)[C@@H]2C[C@@H](O)CN2C(=O)[C@@H](n2cc(C3CC3)nn2)C(C)(C)C)cc1] | (2S,4R)-N-((S)-3-(4-((4-(4′-bromo-5′- oxo-5′H-spiro[cyclohexane-1,7′- indolo[1,2-a]quinazolin]-10′- yl)piperidin-1-yl)methyl)piperidin-1- yl)-1-(4-(4-methylthiazol-5-yl)phenyl)- 3-oxopropyl)-1-((S)-2-(4-cyclopropyl- 1H-1,2,3-triazol-1-yl)-3,3- dimethylbutanoyl)-4- hydroxypyrrolidine-2-carboxamide |
| A142 | 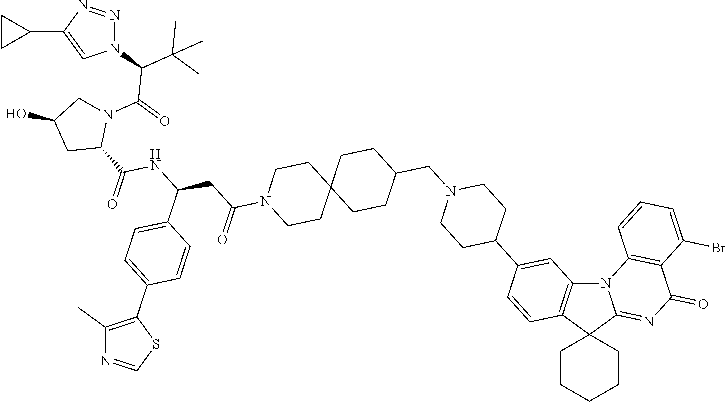 [SMILES: Cc1ncsc1-c1ccc([C@H](CC(=O)N2CCC3(CCC(CN4CCC(c5ccc6c(c5)-n5c(nc(=O)c7c(Br)cccc75)C65CCCCC5)CC4)CC3)CC2)NC(=O)[C@@H]2C[C@@H](O)CN2C(=O)[C@@H](n2cc(C3CC3)nn2)C(C)(C)C)cc1] | (2S,4R)-N-((S)-3-(9-((4-(4′-bromo-5′- oxo-5′H-spiro[cyclohexane-1,7′- indolo[1,2-a]quinazolin]-10′- yl)piperidin-1-yl)methyl)-3- azaspiro[5.5]undecan-3-yl)-1-(4-(4- methylthiazol-5-yl)phenyl)-3- oxopropyl)-1-((S)-2-(4-cyclopropyl- 1H-1,2,3-triazol-1-yl)-3,3- dimethylbutanoyl)-4- hydroxypyrrolidine-2-carboxamide |
| A143 | 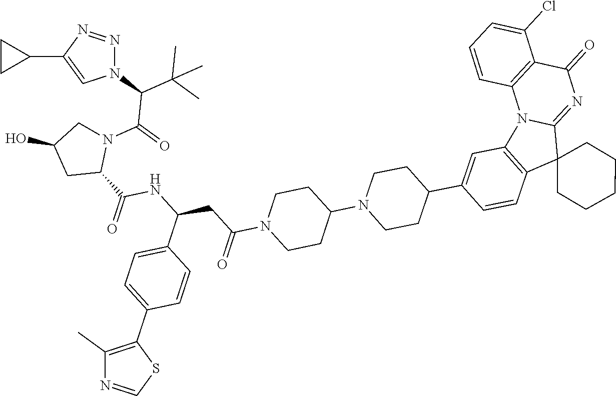 [SMILES: Cc1ncsc1-c1ccc([C@H](CC(=O)N2CCC(N3CCC(c4ccc5c(c4)-n4c(nc(=O)c6c(Cl)cccc64)C54CCCCC4)CC3)CC2)NC(=O)[C@@H]2C[C@@H](O)CN2C(=O)[C@@H](n2cc(C3CC3)nn2)C(C)(C)C)cc1] | (2S,4R)-N-((S)-3-(4-(4′-chloro-5′-oxo- 5′H-spiro[cyclohexane-1,7′-indolo[1,2- a]quinazolin]-10′-yl)-[1,4′-bipiperidin]- 1′-yl)-1-(4-(4-methylthiazol-5- yl)phenyl)-3-oxopropyl)-1-((S)-2-(4- cyclopropyl-1H-1,2,3-triazol-1-yl)-3,3- dimethylbutanoyl)-4- hydroxypyrrolidine-2-carboxamide |
| A144 | 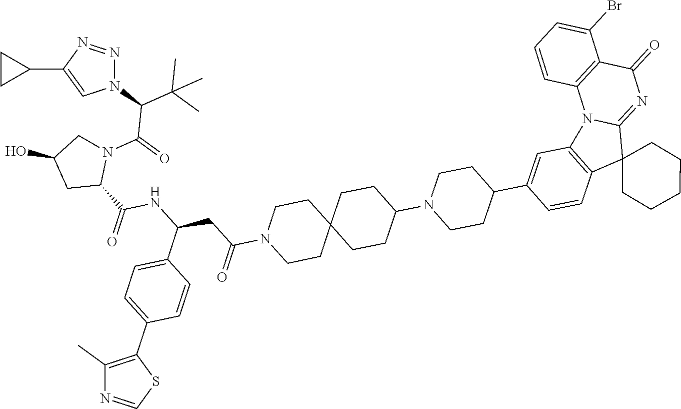 [SMILES: Cc1ncsc1-c1ccc([C@H](CC(=O)N2CCC3(CCC(N4CCC(c5ccc6c(c5)-n5c(nc(=O)c7c(Br)cccc75)C65CCCCC5)CC4)CC3)CC2)NC(=O)[C@@H]2C[C@@H](O)CN2C(=O)[C@@H](n2cc(C3CC3)nn2)C(C)(C)C)cc1] | (2S,4R)-N-((S)-3-(9-(4-(4′-bromo-5′- oxo-5′H-spiro[cyclohexane-1,7′- indolo[1,2-a]quinazolin]-10′- yl)piperidin-1-yl)-3- azaspiro[5.5]undecan-3-yl)-1-(4-(4- methylthiazol-5-yl)phenyl)-3- oxopropyl)-1-((S)-2-(4-cyclopropyl- 1H-1,2,3-triazol-1-yl)-3,3- dimethylbutanoyl)-4- hydroxypyrrolidine-2-carboxamide |
| A145 | 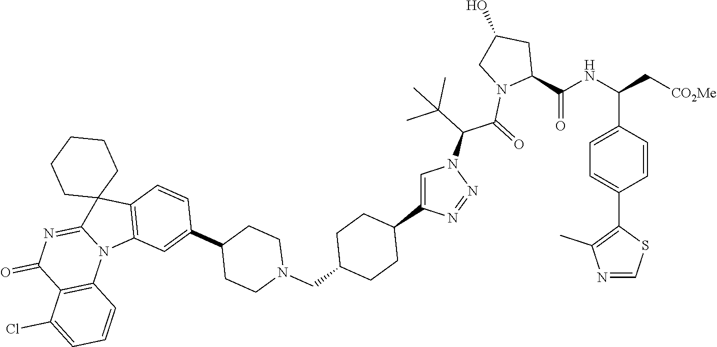 | methyl (S)-3-((2S,4R)-1-((S)-2-(4- ((1r,4S)-4-((4-(4′-chloro-5′-oxo-5′H- spiro[cyclohexane-1,7′-indolo[1,2- a]quinazolin]-10′-yl)piperidin-1- yl)methyl)cyclohexyl)-1H-1,2,3- triazol-1-yl)-3,3-dimethylbutanoyl)-4- hydroxypyrrolidine-2-carboxamido)-3- (4-(4-methylthiazol-5- yl)phenyl)propanoate |
| A146 | 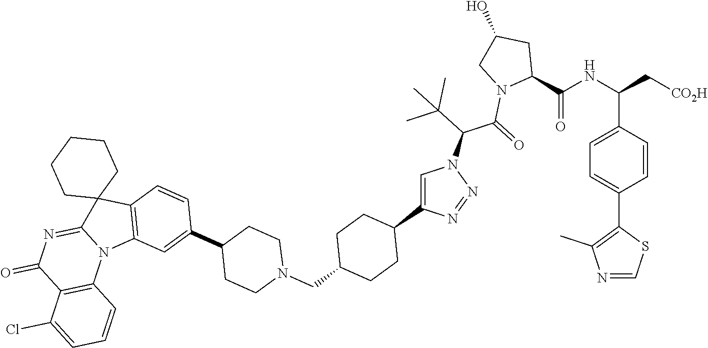 | (S)-3-((2S,4R)-1-((S)-2-(4-((1r,4S)-4- ((4-(4′-chloro-5′-oxo-5′H- spiro[cyclohexane-1,7′-indolo[1,2- a]quinazolin]-10′-yl)piperidin-1- yl)methyl)cyclohexyl)-1H-1,2,3- triazol-1-yl)-3,3-dimethylbutanoyl)-4- hydroxypyrrolidine-2-carboxamido)-3- (4-(4-methylthiazol-5- yl)phenyl)propanoic acid |
| A147 | 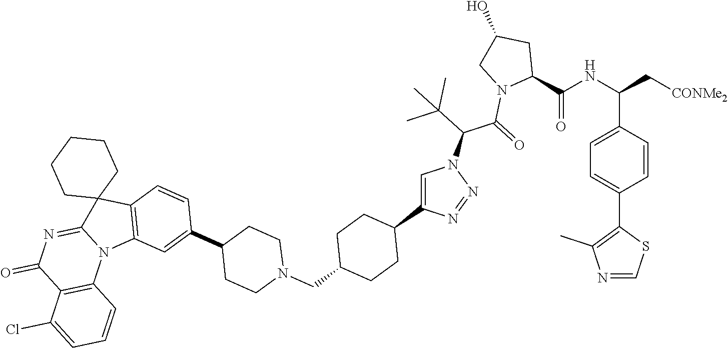 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-N-((S)-3- (dimethylamino)-1-(4-(4- methylthiazol-5-yl)phenyl)-3- oxopropyl)-4-hydroxypyrrolidine-2- carboxamide |
| A148 | 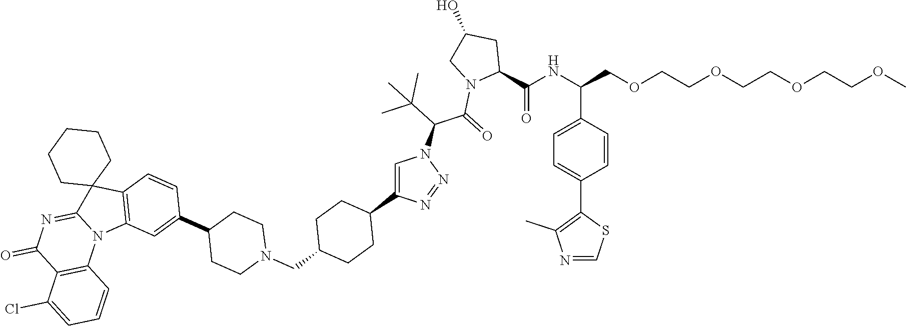 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 13-(4-(4-methylthiazol-5-yl)phenyl)- 2,5,8,11-tetraoxatridecan-13- yl)pyrrolidine-2-carboxamide |
| A149 | 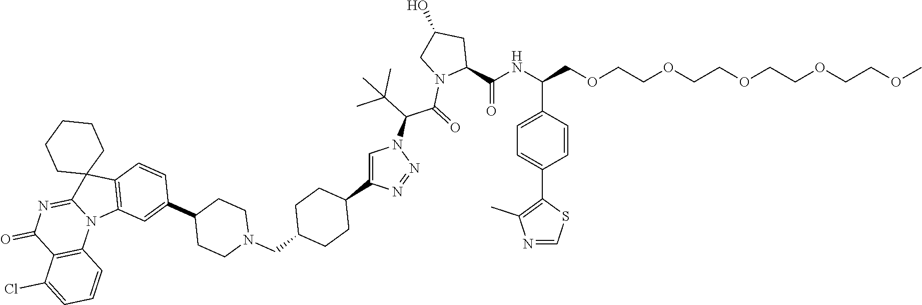 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 16-(4-(4-methylthiazol-5-yl)phenyl)- 2,5,8,11,14-pentaoxahexadecan-16- yl)pyrrolidine-2-carboxamide |
| A150 | 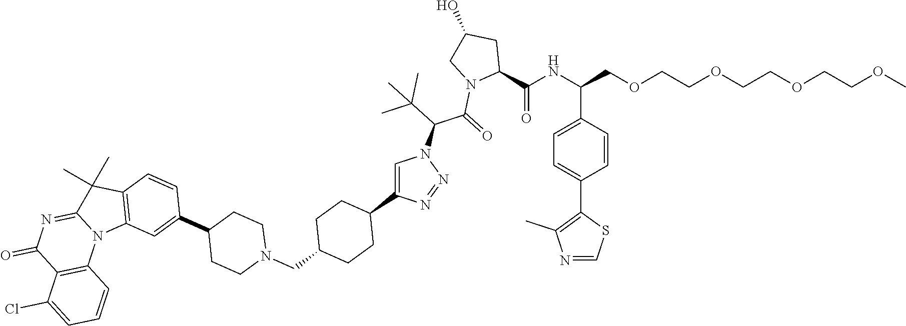 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-3,3- dimethylbutanoyl)-4-hydroxy-N-((R)- 13-(4-(4-methylthiazol-5-yl)phenyl)- 2,5,8,11-tetraoxatridecan-13- yl)pyrrolidine-2-carboxamide |
| A151 | 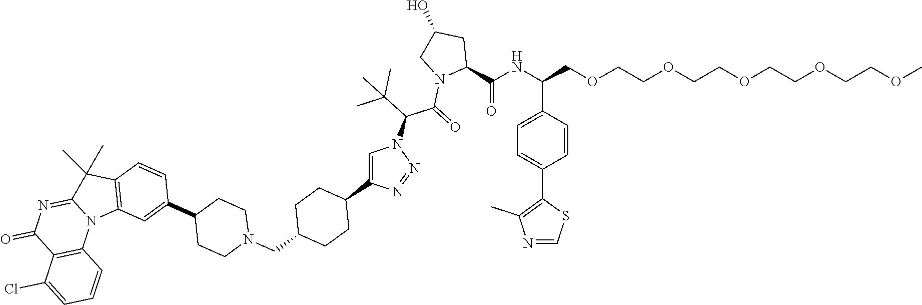 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-3,3- dimethylbutanoyl)-4-hydroxy-N-((R)- 16-(4-(4-methylthiazol-5-yl)phenyl)- 2,5,8,11,14-pentaoxahexadecan-16- yl)pyrrolidine-2-carboxamide |
| A152 | 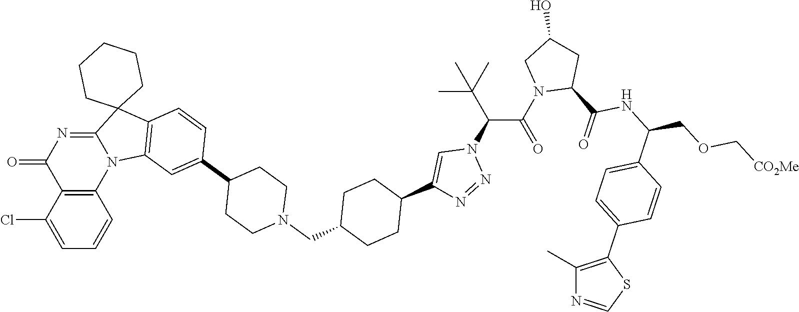 | methyl 2-((R)-2-((2S,4R)-1-((S)-2-(4- ((1r,4S)-4-((4-(4′-chloro-5′-oxo-5′H- spiro[cyclohexane-1,7′-indolo[1,2- a]quinazolin]-10′-yl)piperidin-1- yl)methyl)cyclohexyl)-1H-1,2,3- triazol-1-yl)-3,3-dimethylbutanoyl)-4- hydroxypyrrolidine-2-carboxamido)-2- (4-(4-methylthiazol-5- yl)phenyl)ethoxy)acetate |
| A153 | 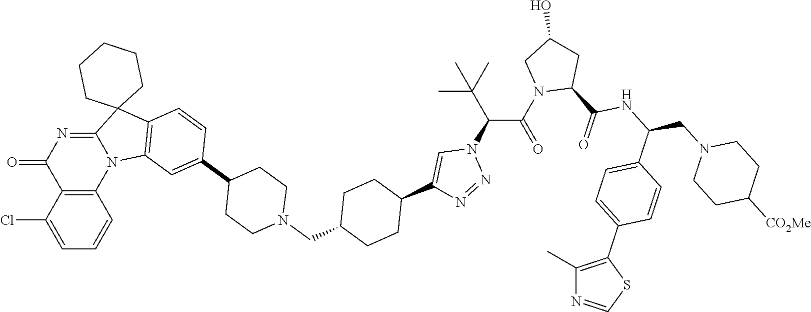 | methyl 1-((R)-2-((2S,4R)-1-((S)-2-(4- ((1r,4S)-4-((4-(4′-chloro-5′-oxo-5′H- spiro[cyclohexane-1,7′-indolo[1,2- a]quinazolin]-10′-yl)piperidin-1- yl)methyl)cyclohexyl)-1H-1,2,3- triazol-1-yl)-3,3-dimethylbutanoyl)-4- hydroxypyrrolidine-2-carboxamido)-2- (4-(4-methylthiazol-5- yl)phenyl)ethyl)piperidine-4- carboxylate |
| A154 | 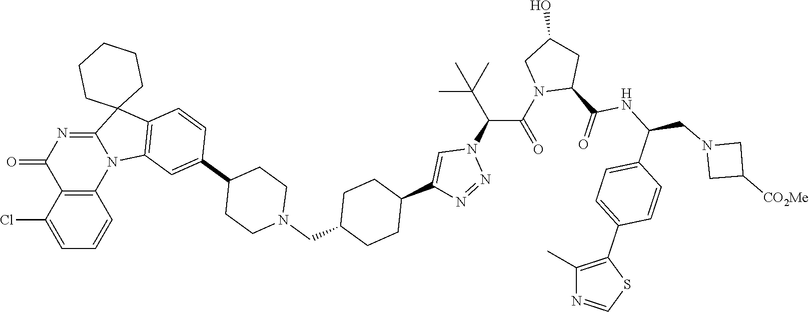 | methyl 1-((R)-2-((2S,4R)-1-((S)-2-(4- ((1r,4S)-4-((4-(4′-chloro-5′-oxo-5′H- spiro[cyclohexane-1,7′-indolo[1,2- a]quinazolin]-10′-yl)piperidin-1- yl)methyl)cyclohexyl)-1H-1,2,3- triazol-1-yl)-3,3-dimethylbutanoyl)-4- hydroxypyrrolidine-2-carboxamido)-2- (4-(4-methylthiazol-5- yl)phenyl)ethyl)azetidine-3- carboxylate |
| A155 | 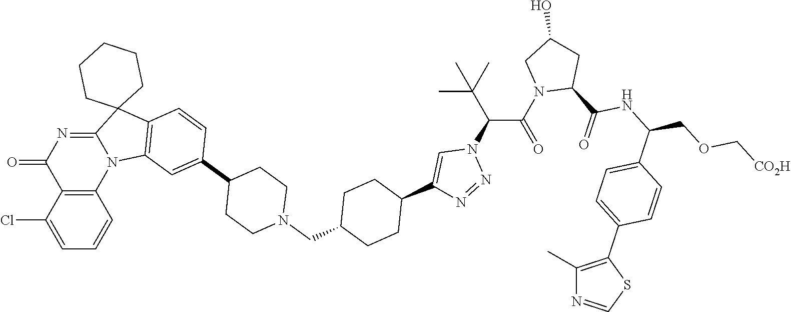 | 2-((R)-2-((2S,4R)-1-((S)-2-(4-((1r,4S)- 4-((4-(4′-chloro-5′-oxo-5′H- spiro[cyclohexane-1,7′-indolo[1,2- a]quinazolin]-10′-yl)piperidin-1- yl)methyl)cyclohexyl)-1H-1,2,3- triazol-1-yl)-3,3-dimethylbutanoyl)-4- hydroxypyrrolidine-2-carboxamido)-2- (4-(4-methylthiazol-5- yl)phenyl)ethoxy)acetic acid |
| A156 | 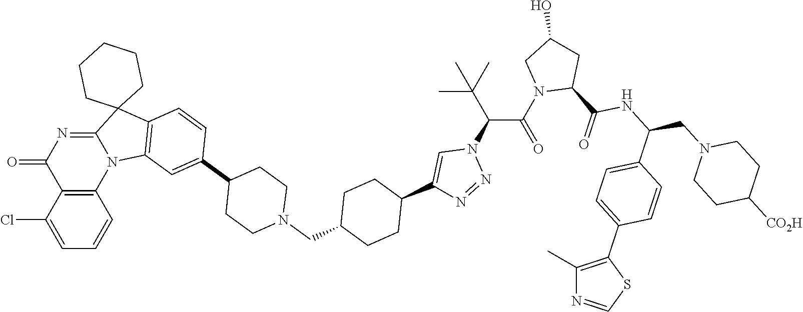 | 1-((R)-2-((2S,4R)-1-((S)-2-(4-((1r,4S)- 4-((4-(4′-chloro-5′-oxo-5′H- spiro[cyclohexane-1,7′-indolo[1,2- a]quinazolin]-10′-yl)piperidin-1- yl)methyl)cyclohexyl)-1H-1,2,3- triazol-1-yl)-3,3-dimethylbutanoyl)-4- hydroxypyrrolidine-2-carboxamido)-2- (4-(4-methylthiazol-5- yl)phenyl)ethyl)piperidine-4- carboxylic acid |
| A157 | 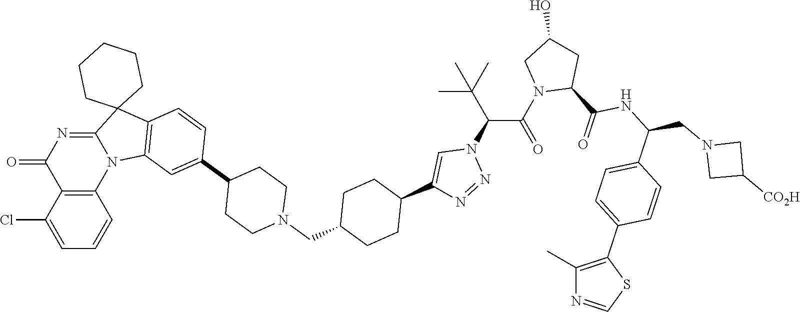 | 1-((R)-2-((2S,4R)-1-((S)-2-(4-((1r,4S)- 4-((4-(4′-chloro-5′-oxo-5′H- spiro[cyclohexane-1,7′-indolo[1,2- a]quinazolin]-10′-yl)piperidin-1- yl)methyl)cyclohexyl)-1H-1,2,3- triazol-1-yl)-3,3-dimethylbutanoyl)-4- hydroxypyrrolidine-2-carboxamido)-2- (4-(4-methylthiazol-5- yl)phenyl)ethyl)azetidine-3-carboxylic acid |
| A158 | 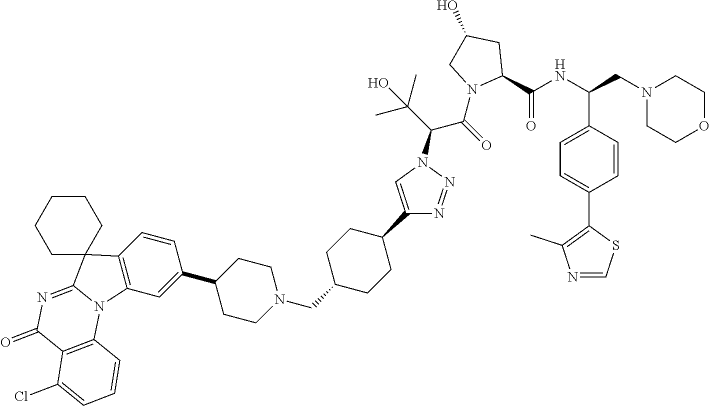 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3-hydroxy-3- methylbutanoyl)-4-hydroxy-N-((R)-1- (4-(4-methylthiazol-5-yl)phenyl)-2- morpholinoethyl)pyrrolidine-2- carboxamide |
| A159 | 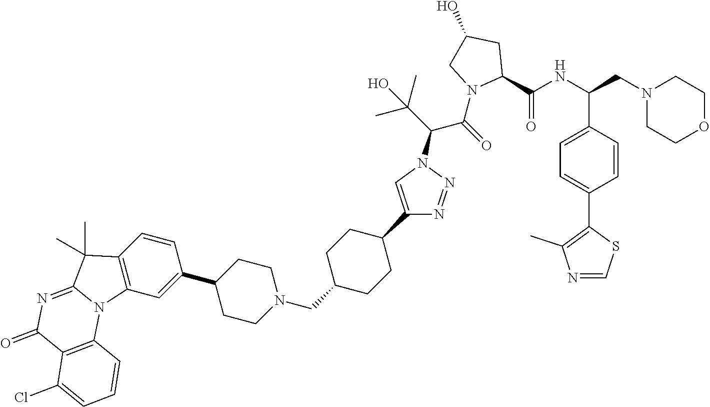 | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)- 1H-1,2,3-triazol-1-yl)-3-hydroxy-3- methylbutanoyl)-4-hydroxy-N-((R)-1- (4-(4-methylthiazol-5-yl)phenyl)-2- morpholinoethyl)pyrrolidine-2- carboxamide |
| A160 | 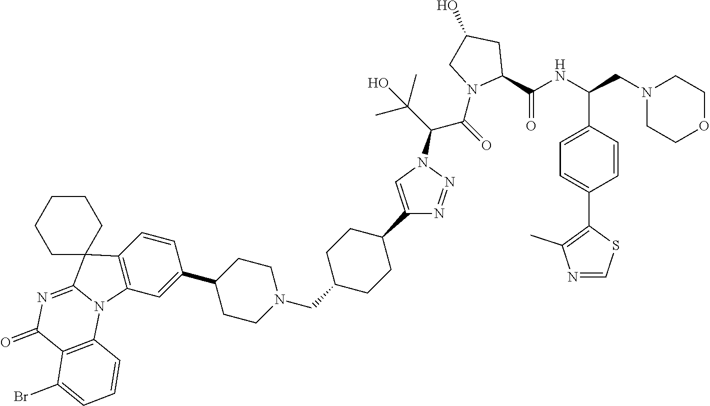 | (2S,4R)-1-((S)-2-(4-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3-hydroxy-3- methylbutanoyl)-4-hydroxy-N-((R)-1- (4-(4-methylthiazol-5-yl)phenyl)-2- morpholinoethyl)pyrrolidine-2- carboxamide + |
| A161 | 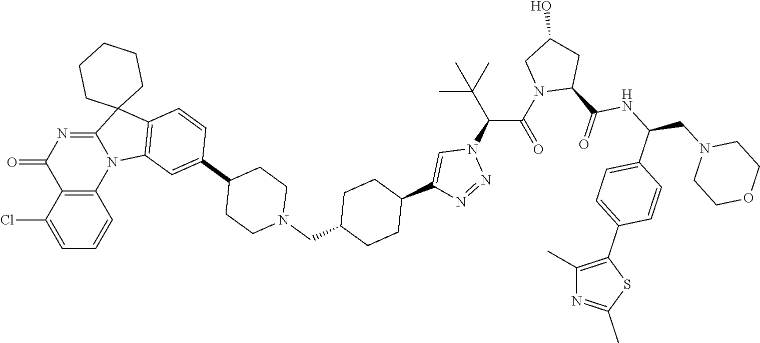 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-N-((R)-1-(4-(2,4- dimethylthiazol-5-yl)phenyl)-2- morpholinoethyl)-4- hydroxypyrrolidine-2-carboxamide |
| A162 | 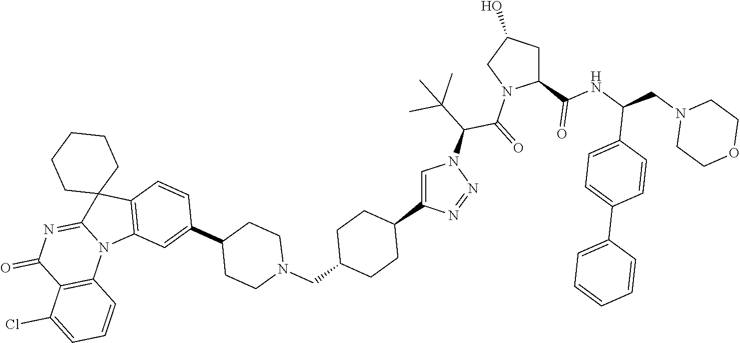 | (2S,4R)-N-((R)-1-([1,1′-biphenyl]-4- yl)-2-morpholinoethyl)-1-((S)-2-(4- ((1r,4S)-4-((4-(4′-chloro-5′-oxo-5′H- spiro[cyclohexane-1,7′-indolo[1,2- a]quinazolin]-10′-yl)piperidin-1- yl)methyl)cyclohexyl)-1H-1,2,3- triazol-1-yl)-3,3-dimethylbutanoyl)-4- hydroxypyrrolidine-2-carboxamide |
| A163 | 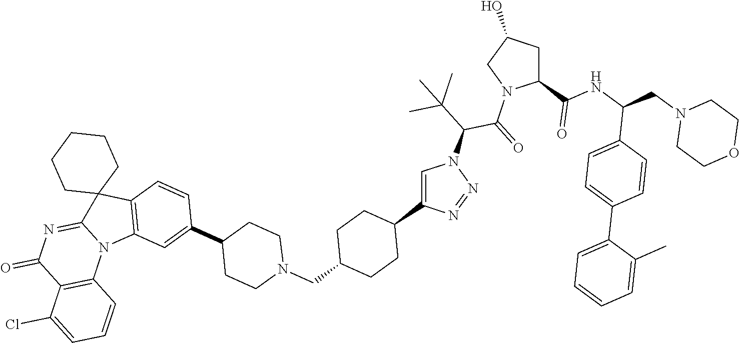 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 1-(2′-methyl-[1,1′-biphenyl]-4-yl)-2- morpholinoethyl)pyrrolidine-2- carboxamide |
| A164 | 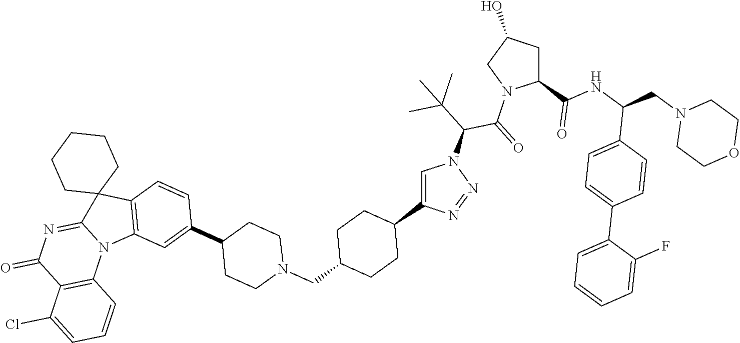 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-N-((R)-1-(2′-fluoro- [1,1′-biphenyl]-4-yl)-2- morpholinoethyl)-4- hydroxypyrrolidine-2-carboxamide |
| A165 | 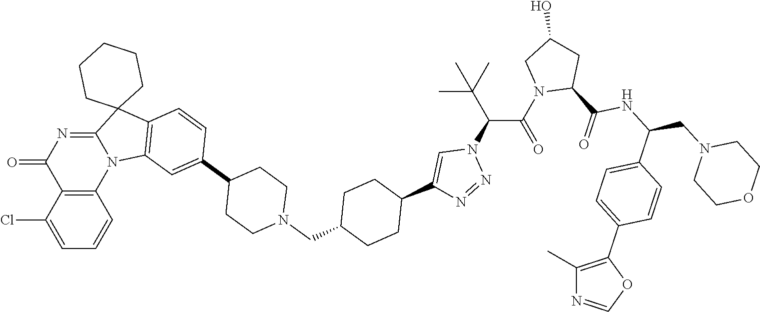 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 1-(4-(4-methyloxazol-5-yl)phenyl)-2- morpholinoethyl)pyrrolidine-2- carboxamide |
| A166 | 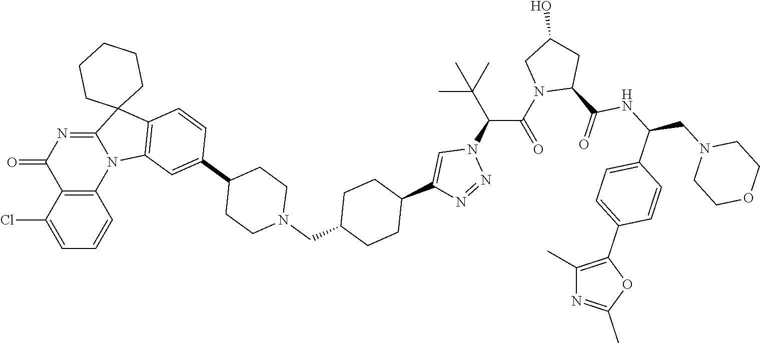 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-N-((R)-1-(4-(2,4- dimethyloxazol-5-yl)phenyl)-2- morpholinoethyl)-4- hydroxypyrrolidine-2-carboxamide |
| A167 | 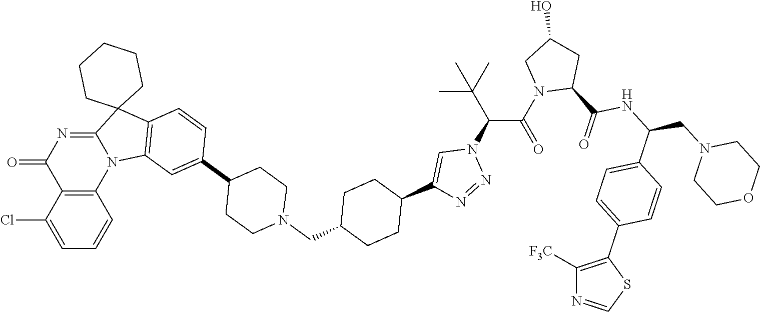 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 2-morpholino-1-(4-(4- (trifluoromethyl)thiazol-5- yl)phenyl)ethyl)pyrrolidine-2- carboxamide |
| A168 | 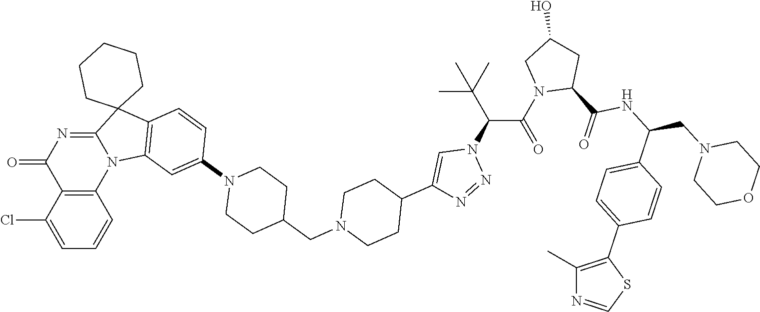 | (2S,4R)-1-((S)-2-(4-(1-((1-(4′-chloro- 5′-oxo-5′H-spiro[cyclohexane-1,7′- indolo[1,2-a]quinazolin]-10′- yl)piperidin-4-yl)methyl)piperidin-4- yl)-1H-1,2,3-triazol-1-yl)-3,3- dimethylbutanoyl)-4-hydroxy-N-((R)- 1-(4-(4-methylthiazol-5-yl)phenyl)-2- morpholinoethyl)pyrrolidine-2- carboxamide |
| A169 | 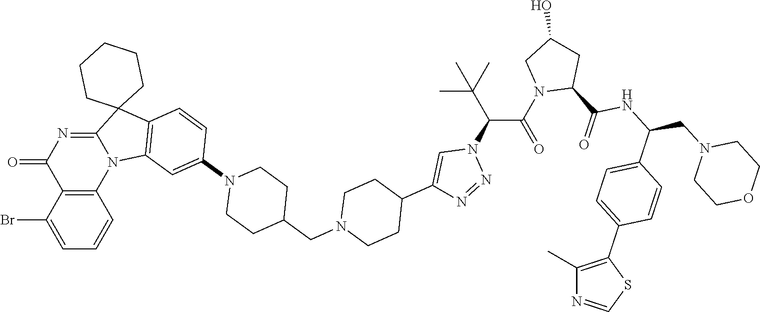 | (2S,4R)-1-((S)-2-(4-(1-((1-(4′-bromo- 5′-oxo-5′H-spiro[cyclohexane-1,7′- indolo[1,2-a]quinazolin]-10′- yl)piperidin-4-yl)methyl)piperidin-4- yl)-1H-1,2,3-triazol-1-yl)-3,3- dimethylbutanoyl)-4-hydroxy-N-((R)- 1-(4-(4-methylthiazol-5-yl)phenyl)-2- morpholinoethyl)pyrrolidine-2- carboxamide |
| A170 | 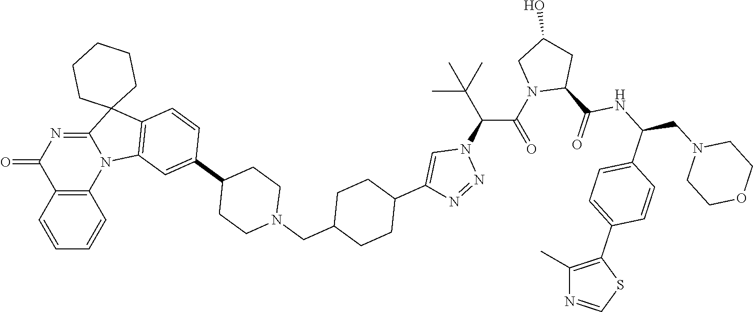 | (2S,4R)-1-((S)-3,3-dimethyl-2-(4-(4- ((4-(5′-oxo-5′H-spiro[cyclohexane-1,7′- indolo[1,2-a]quinazolin]-10′- yl)piperidin-1-yl)methyl)cyclohexyl)- 1H-1,2,3-triazol-1-yl)butanoyl)-4- hydroxy-N-((R)-1-(4-(4-methylthiazol- 5-yl)phenyl)-2- morpholinoethyl)pyrrolidine-2- carboxamide |
| A171 | 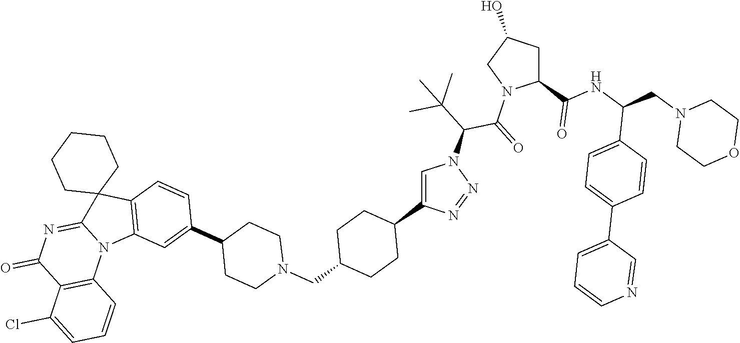 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 2-morpholino-1-(4-(pyridin-3- yl)phenyl)ethyl)pyrrolidine-2- carboxamide |
| A172 | 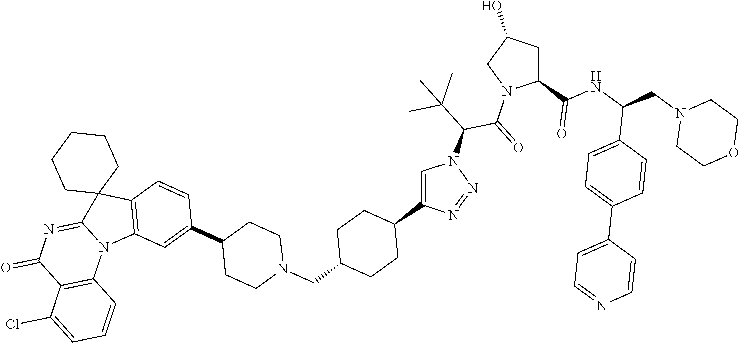 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 2-morpholino-1-(4-(pyridin-4- yl)phenyl)ethyl)pyrrolidine-2- carboxamide |
| A173 | 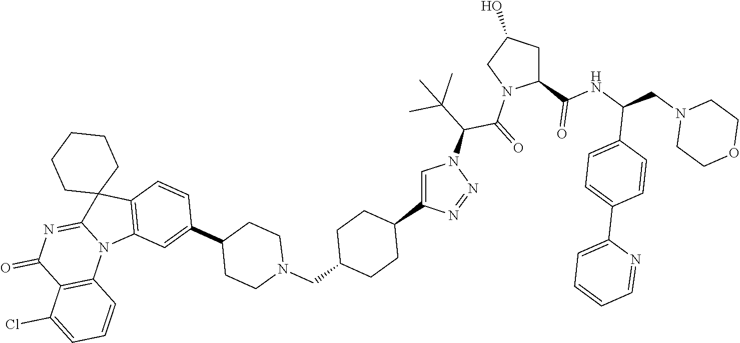 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 2-morpholino-1-(4-(pyridin-2- yl)phenyl)ethyl)pyrrolidine-2- carboxamide |
| A174 | 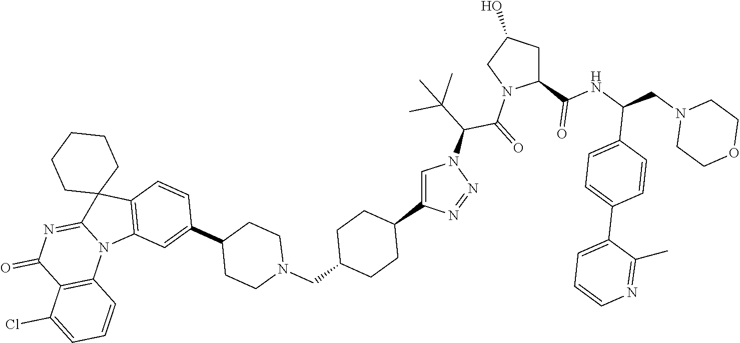 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 1-(4-(2-methylpyridin-3-yl)phenyl)-2- morpholinoethyl)pyrrolidine-2- carboxamide |
| A175 | 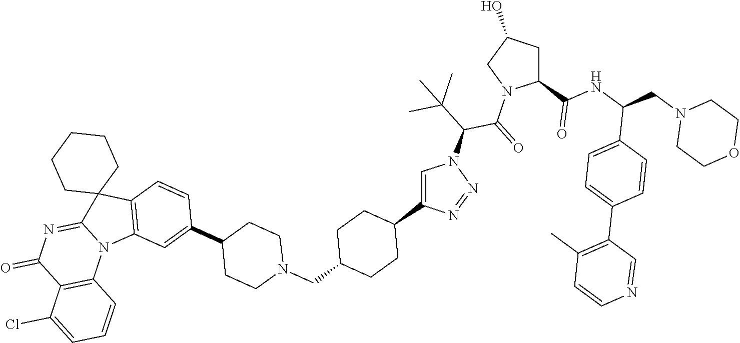 | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)- 1H-1,2,3-triazol-1-yl)-3,3- dimethylbutanoyl)-4-hydroxy-N-((R)- 1-(4-(4-methylpyridin-3-yl)phenyl)-2- morpholinoethyl)pyrrolidine-2- carboxamide |

TABLE 2

| Compound | | |
|---|---|---|
| No. | Structure | Chemical Name |
| B1 | 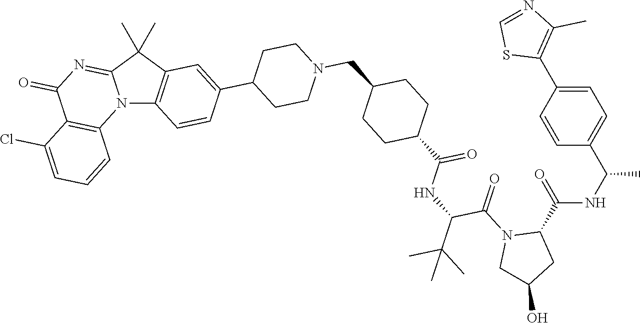 [SMILES: Cc1ncsc1-c1ccc([C@H](C)NC(=O)[C@@H]2C[C@@H](O)CN2C(=O)[C@@H](NC(=O)[C@H]2CC[C@H](CN3CCC(c4ccc5c(c4)C(C)(C)c4nc(=O)c6c(Cl)cccc6n4-5)CC3)CC2)C(C)(C)C)cc1] | (2S,4R)-1-((S)-2-((11,4S)-4-((4-(4- chloro-7,7-dimethyl-5-oxo-5,7- dihydroindolo[1,2-a]quinazolin-9- yl)piperidin-1-yl)methyl)cyclohexane-1- carboxamido)-3,3-dimethylbutanoyl)-4- hydroxy-N-((S)-1-(4-(4-methylthiazol-5- yl)phenyl)ethyl)pyrrolidine-2- carboxamide |
| B2 | 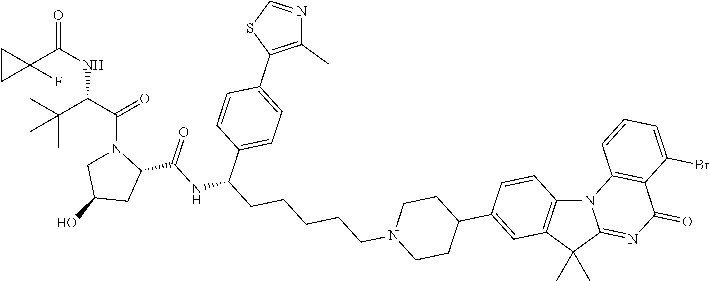 [SMILES: Cc1ncsc1-c1ccc([C@H](CCCCCN2CCC(c3ccc4c(c3)C(C)(C)c3nc(=O)c5c(Br)cccc5n3-4)CC2)NC(=O)[C@@H]2C[C@@H](O)CN2C(=O)[C@@H](NC(=O)C2(F)CC2)C(C)(C)C)cc1] | (2S,4R)-N-((S)-6-(4-(4-bromo-7,7- dimethyl-5-oxo-5,7-dihydroindolo[1,2-a] quinazolin-9-yl)piperidin-1-yl)-1-(4-(4- methylthiazol-5-yl)phenyl)hexyl)-1-((S)- 2-(1-fluorocyclopropane-1- carboxamido)-3,3-dimethylbutanoyl)-4- hydroxypyrrolidine-2-carboxamide |
| B3 | 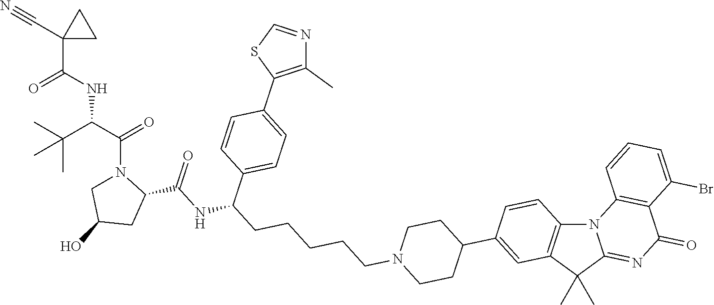 [SMILES: Cc1ncsc1-c1ccc([C@H](CCCCCN2CCC(c3ccc4c(c3)C(C)(C)c3nc(=O)c5c(Br)cccc5n3-4)CC2)NC(=O)[C@@H]2C[C@@H](O)CN2C(=O)[C@@H](NC(=O)C2(C#N)CC2)C(C)(C)C)cc1] | (2S,4R)-N-((S)-6-(4-(4-bromo-7,7- dimethyl-5-oxo-5,7-dihydroindolo[1,2- a]quinazolin-9-yl)piperidin-1-yl)-1-(4-(4- methylthiazol-5-yl)phenyl)hexyl)-1-((S)- 2-(1-cyanocyclopropane-1-carboxamido)- 3,3-dimethylbutanoyl)-4- hydroxypyrrolidine-2-carboxamide |
| B4 | 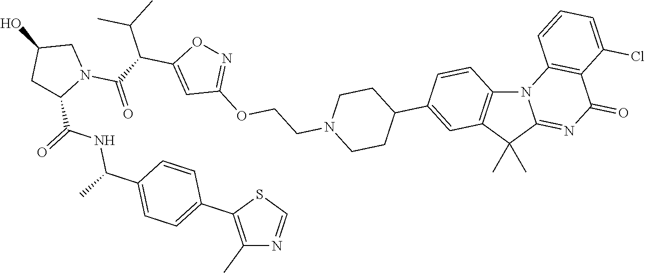 [SMILES: Cc1ncsc1-c1ccc([C@H](C)NC(=O)[C@@H]2C[C@@H](O)CN2C(=O)[C@@H](c2cc(OCCN3CCC(c4ccc5c(c4)C(C)(C)c4nc(=O)c6c(Cl)cccc6n4-5)CC3)no2)C(C)C)cc1] | (2S,4R)-1-((R)-2-(3-(2-(4-(4-chloro-7,7- dimethyl-5-oxo-5,7-dihydroindolo[1,2- a]quinazolin-9-yl)piperidin-1- yl)ethoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B5 | 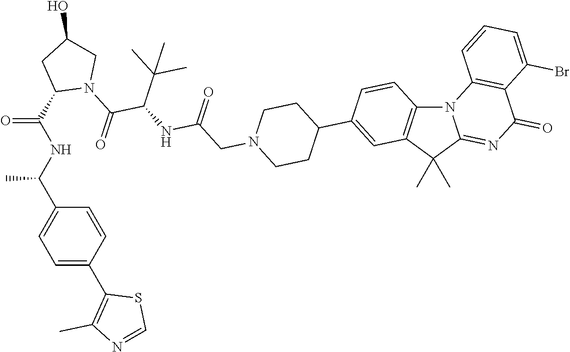 [SMILES: Cc1ncsc1-c1ccc([C@H](C)NC(=O)[C@@H]2C[C@@H](O)CN2C(=O)[C@@H](NC(=O)CN2CCC(c3ccc4c(c3)C(C)(C)c3nc(=O)c5c(Br)cccc5n3-4)CC2)C(C)(C)C)cc1] | (2S,4R)-1-((S)-2-(2-(4-(4-bromo-7,7- dimethyl-5-oxo-5,7-dihydroindolo[1,2-a] quinazolin-9-yl)piperidin-1- yl)acetamido)-3,3-dimethylbutanoyl)-4- hydroxy-N-((S)-1-(4-(4-methylthiazol-5- yl)phenyl)ethyl)pyrrolidine-2- carboxamide |
| B6 | 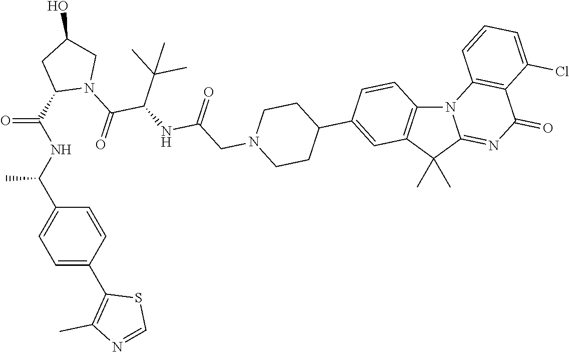 [SMILES: Cc1ncsc1-c1ccc([C@H](C)NC(=O)[C@@H]2C[C@@H](O)CN2C(=O)[C@@H](NC(=O)CN2CCC(c3ccc4c(c3)C(C)(C)c3nc(=O)c5c(Cl)cccc5n3-4)CC2)C(C)(C)C)cc1] | (2S,4R)-1-((S)-2-(2-(4-(4-chloro-7,7- dimethyl-5-oxo-5,7-dihydroindolo[1,2- a]quinazolin-9-yl)piperidin-1- yl)acetamido)-3,3-dimethylbutanoyl)-4- hydroxy-N-((S)-1-(4-(4-methylthiazol-5- yl)phenyl)ethyl)pyrrolidine-2- carboxamide |
| B7 | 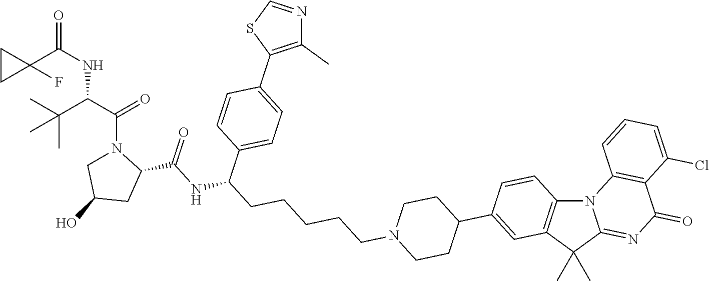 [SMILES: Cc1ncsc1-c1ccc([C@H](CCCCCN2CCC(c3ccc4c(c3)C(C)(C)c3nc(=O)c5c(Cl)cccc5n3-4)CC2)NC(=O)[C@@H]2C[C@@H](O)CN2C(=O)[C@@H](NC(=O)C2(F)CC2)C(C)(C)C)cc1] | (2S,4R)-N-((S)-6-(4-(4-chloro-7,7- dimethyl-5-oxo-5,7-dihydroindolo[1,2- a]quinazolin-9-yl)piperidin-1-yl)-1-(4-(4- methylthiazol-5-yl)phenyl)hexyl)-1-((S)- 2-(1-fluorocyclopropane-1- carboxamido)-3,3-dimethylbutanoyl)-4- hydroxypyrrolidine-2-carboxamide |
| B8 | 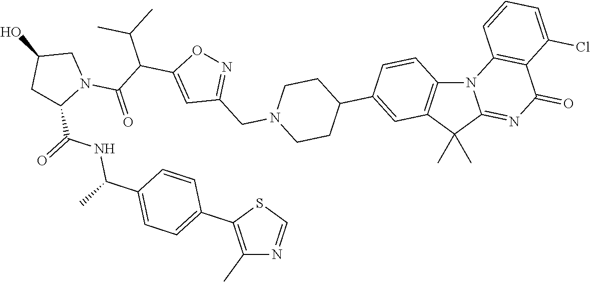 [SMILES: Cc1ncsc1-c1ccc([C@H](C)NC(=O)[C@@H]2C[C@@H](O)CN2C(=O)C(c2cc(CN3CCC(c4ccc5c(c4)C(C)(C)c4nc(=O)c6c(Cl)cccc6n4-5)CC3)no2)C(C)C)cc1] | (2S,4R)-1-(2-(3-((4-(4-chloro-7,7- dimethyl-5-oxo-5,7-dihydroindolo[1,2- a]quinazolin-9-yl)piperidin-1- yl)methyl)isoxazol-5-yl)-3- methylbutanoyl)-4-hydroxy-N-((S)-1-(4- (4-methylthiazol-5- yl)phenyl)ethyl)pyrrolidine-2- carboxamide |
| B9 | 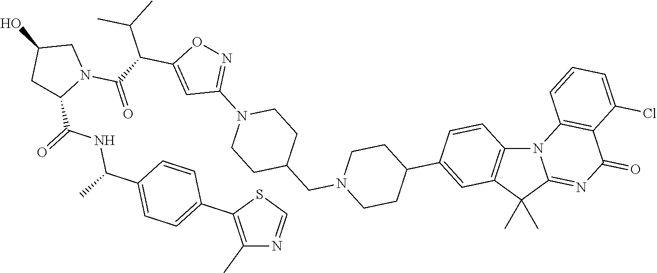 [SMILES: Cc1ncsc1-c1ccc([C@H](C)NC(=O)[C@@H]2C[C@@H](O)CN2C(=O)[C@@H](c2cc(N3CCC(CN4CCC(c5ccc6c(c5)C(C)(C)c5nc(=O)c7c(Cl)cccc7n5-6)CC4)CC3)no2)C(C)C)cc1] | (2S,4R)-1-((R)-2-(3-(4-((4-(4-chloro-7,7- dimethyl-5-oxo-5,7-dihydroindolo[1,2- a]quinazolin-9-yl)piperidin-1- yl)methyl)piperidin-1-yl)isoxazol-5-yl)- 3-methylbutanoyl)-4-hydroxy-N-((S)-1- (4-(4-methylthiazol-5- yl)phenyl)ethyl)pyrrolidine-2- carboxamide |
| B10 | 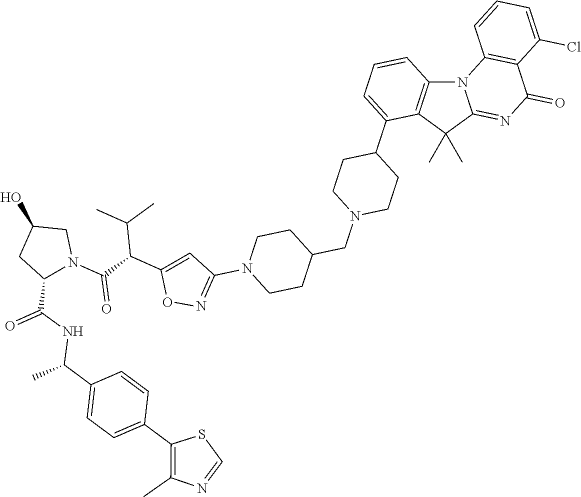 [SMILES: Cc1ncsc1-c1ccc([C@H](C)NC(=O)[C@@H]2C[C@@H](O)CN2C(=O)[C@@H](c2cc(N3CCC(CN4CCC(c5cccc6c5C(C)(C)c5nc(=O)c7c(Cl)cccc7n5-6)CC4)CC3)no2)C(C)C)cc1] | (2S,4R)-1-((R)-2-(3-(4-((4-(4-chloro-7,7- dimethyl-5-oxo-5,7-dihydroindolo[1,2- a]quinazolin-8-yl)piperidin-1- yl)methyl)piperidin-1-yl)isoxazol-5-yl)- 3-methylbutanoyl)-4-hydroxy-N-((S)-1- (4-(4-methylthiazol-5- yl)phenyl)ethyl)pyrrolidine-2- carboxamide |
| B11 | 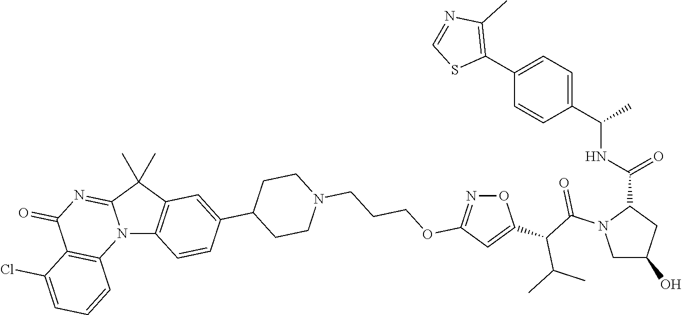 | (2S,4R)-1-((R)-2-(3-(3-(4-(4-chloro-7,7- dimethyl-5-oxo-5,7-dihydroindolo[1,2- a]quinazolin-9-yl)piperidin-1- yl)propoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B12 | 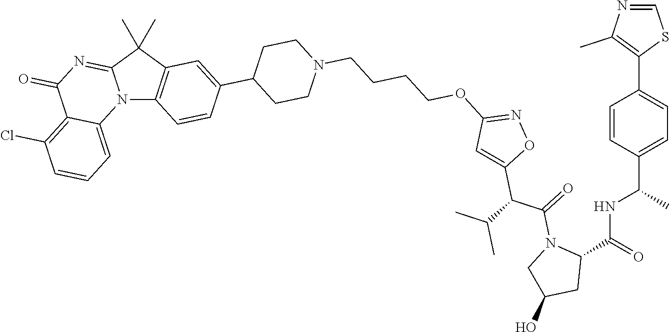 [SMILES: Cc1ncsc1-c1ccc([C@H](C)NC(=O)[C@@H]2C[C@@H](O)CN2C(=O)[C@@H](c2cc(OCCCCN3CCC(c4ccc5c(c4)C(C)(C)c4nc(=O)c6c(Cl)cccc6n4-5)CC3)no2)C(C)C)cc1] | (2S,4R)-1-((R)-2-(3-(4-(4-(4-chloro-7,7- dimethyl-5-oxo-5,7-dihydroindolo[1,2- a]quinazolin-9-yl)piperidin-1- yl)butoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B13 | 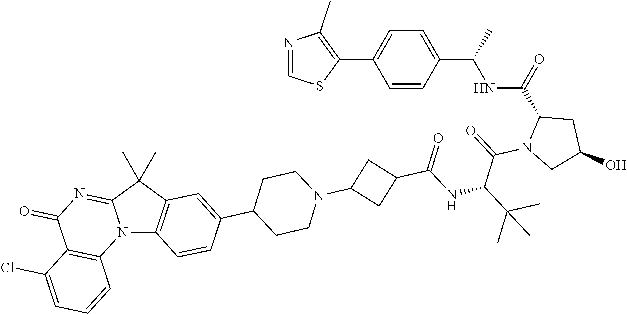 [SMILES: Cc1ncsc1-c1ccc([C@H](C)NC(=O)[C@@H]2C[C@@H](O)CN2C(=O)[C@@H](NC(=O)C2CC(N3CCC(c4ccc5c(c4)C(C)(C)c4nc(=O)c6c(Cl)cccc6n4-5)CC3)C2)C(C)(C)C)cc1] | (2S,4R)-1-((2S)-2-(3-(4-(4-chloro-7,7- dimethyl-5-oxo-5,7-dihydroindolo[1,2- a]quinazolin-9-yl)piperidin-1- yl)cyclobutane-1-carboxamido)-3,3- dimethylbutanoyl)-4-hydroxy-N-((S)-1- (4-(4-methylthiazol-5- yl)phenyl)ethyl)pyrrolidine-2- carboxamide |
| B14 | 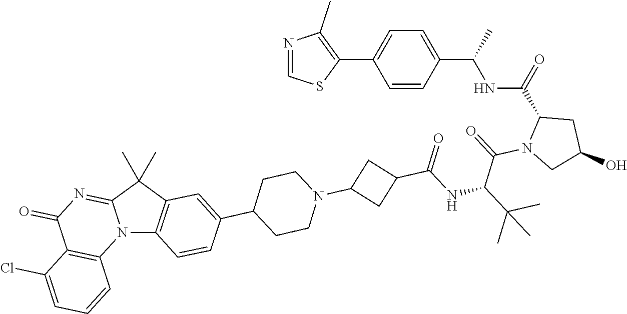 [SMILES: Cc1ncsc1-c1ccc([C@H](C)NC(=O)[C@@H]2C[C@@H](O)CN2C(=O)[C@@H](NC(=O)C2CC(N3CCC(c4ccc5c(c4)C(C)(C)c4nc(=O)c6c(Cl)cccc6n4-5)CC3)C2)C(C)(C)C)cc1] | (2S,4R)-1-((2S)-2-(3-(4-(4-chloro-7,7- dimethyl-5-oxo-5,7-dihydroindolo[1,2- a]quinazolin-9-yl)piperidin-1- yl)cyclobutane-1-carboxamido)-3,3- dimethylbutanoyl)-4-hydroxy-N-((S)-1- (4-(4-methylthiazol-5- yl)phenyl)ethyl)pyrrolidine-2- carboxamide |
| B15 | 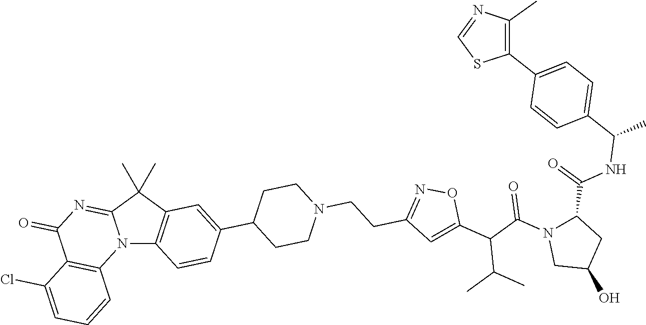 [SMILES: Cc1ncsc1-c1ccc([C@H](C)NC(=O)[C@@H]2C[C@@H](O)CN2C(=O)C(c2cc(CCN3CCC(c4ccc5c(c4)C(C)(C)c4nc(=O)c6c(Cl)cccc6n4-5)CC3)no2)C(C)C)cc1] | (2S,4R)-1-(2-(3-(2-(4-(4-chloro-7,7- dimethyl-5-oxo-5,7-dihydroindolo[1,2- a]quinazolin-9-yl)piperidin-1- yl)ethyl)isoxazol-5-yl)-3- methylbutanoyl)-4-hydroxy-N-((S)-1-(4- (4-methylthiazol-5- yl)phenyl)ethyl)pyrrolidine-2- carboxamide |
| B16 | 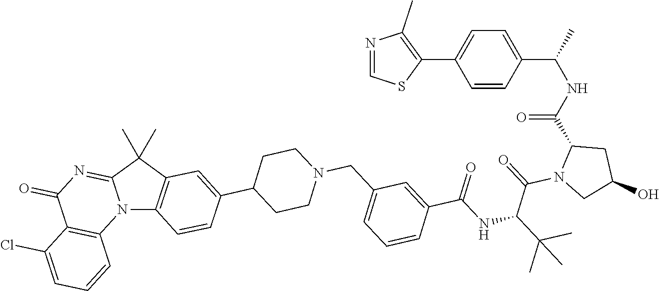 [SMILES: Cc1ncsc1-c1ccc([C@H](C)NC(=O)[C@@H]2C[C@@H](O)CN2C(=O)[C@@H](NC(=O)c2cccc(CN3CCC(c4ccc5c(c4)C(C)(C)c4nc(=O)c6c(Cl)cccc6n4-5)CC3)c2)C(C)(C)C)cc1] | (2S,4R)-1-((S)-2-(3-((4-(4-chloro-7,7- dimethyl-5-oxo-5,7-dihydroindolo[1,2- a]quinazolin-9-yl)piperidin-1- yl)methyl)benzamido)-3,3- dimethylbutanoyl)-4-hydroxy-N-((S)-1- (4-(4-methylthiazol-5- yl)phenyl)ethyl)pyrrolidine-2- carboxamide |
| B17 | 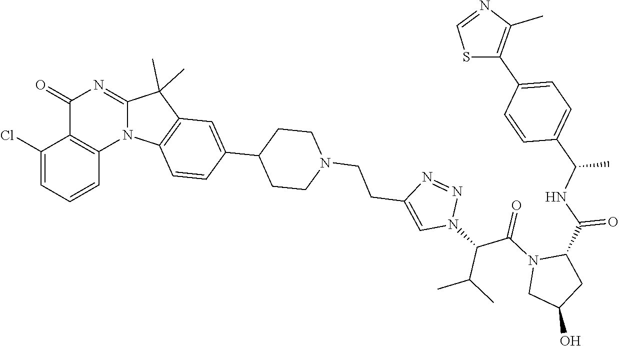 [SMILES: Cc1ncsc1-c1ccc([C@H](C)NC(=O)[C@@H]2C[C@@H](O)CN2C(=O)[C@H](C(C)C)n2cc(CCN3CCC(c4ccc5c(c4)C(C)(C)c4nc(=O)c6c(Cl)cccc6n4-5)CC3)nn2)cc1] | (2S,4R)-1-((S)-2-(4-(2-(4-(4-chloro-7,7- dimethyl-5-oxo-5,7-dihydroindolo[1,2- a]quinazolin-9-yl)piperidin-1-yl)ethyl)- 1H-1,2,3-triazol-1-yl)-3-methylbutanoyl)- 4-hydroxy-N-((S)-1-(4-(4-methylthiazol- 5-yl)phenyl)ethyl)pyrrolidine-2- carboxamide |
| B18 | 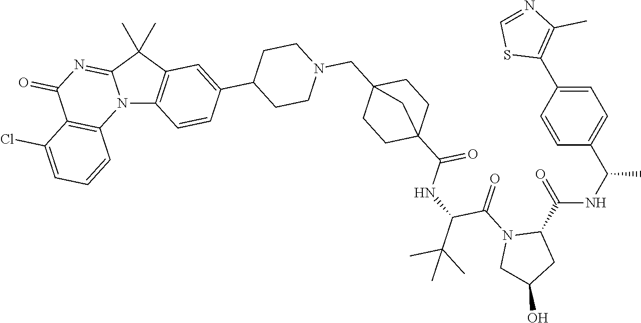 [SMILES: Cc1ncsc1-c1ccc([C@H](C)NC(=O)[C@@H]2C[C@@H](O)CN2C(=O)[C@@H](NC(=O)C23CCC(CN4CCC(c5ccc6c(c5)C(C)(C)c5nc(=O)c7c(Cl)cccc7n5-6)CC4)(CC2)C3)C(C)(C)C)cc1] | (2S,4R)-1-((S)-2-(4-((4-(4-chloro-7,7- dimethyl-5-oxo-5,7-dihydroindolo[1,2- a]quinazolin-9-yl)piperidin-1- yl)methyl)bicyclo[2.2.1]heptane-1- carboxamido)-3,3-dimethylbutanoyl)-4- hydroxy-N-((S)-1-(4-(4-methylthiazol-5- yl)phenyl)ethyl)pyrrolidine-2- carboxamide |
| B19 | 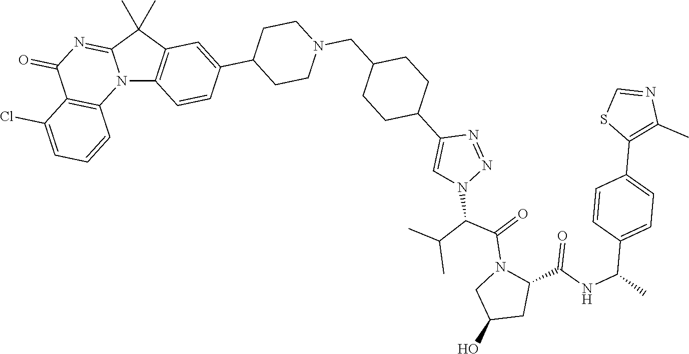 [SMILES: Cc1ncsc1-c1ccc([C@H](C)NC(=O)[C@@H]2C[C@@H](O)CN2C(=O)[C@H](C(C)C)n2cc(C3CCC(CN4CCC(c5ccc6c(c5)C(C)(C)c5nc(=O)c7c(Cl)cccc7n5-6)CC4)CC3)nn2)cc1] | (2S,4R)-1-((S)-2-(4-(4-((4-(4-chloro-7,7- dimethyl-5-oxo-5,7-dihydroindolo[1,2- a]quinazolin-9-yl)piperidin-1- yl)methyl)cyclohexyl)-1H-1,2,3-triazol- 1-yl)-3-methylbutanoyl)-4-hydroxy-N- ((S)-1-(4-(4-methylthiazol-5- yl)phenyl)ethyl)pyrrolidine-2- carboxamide |
| B20 | 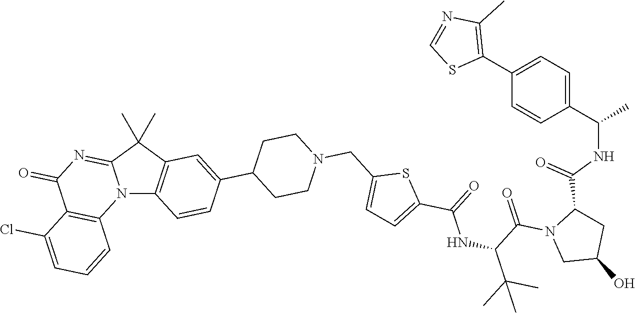 [SMILES: Cc1ncsc1-c1ccc([C@H](C)NC(=O)[C@@H]2C[C@@H](O)CN2C(=O)[C@@H](NC(=O)c2ccc(CN3CCC(c4ccc5c(c4)C(C)(C)c4nc(=O)c6c(Cl)cccc6n4-5)CC3)s2)C(C)(C)C)cc1] | (2S,4R)-1-((S)-2-(5-((4-(4-chloro-7,7- dimethyl-5-oxo-5,7-dihydroindolo[1,2- a]quinazolin-9-yl)piperidin-1- yl)methyl)thiophene-2-carboxamido)-3,3- dimethylbutanoyl)-4-hydroxy-N-((S)-1- (4-(4-methylthiazol-5- yl)phenyl)ethyl)pyrrolidine-2- carboxamide |
| B21 | 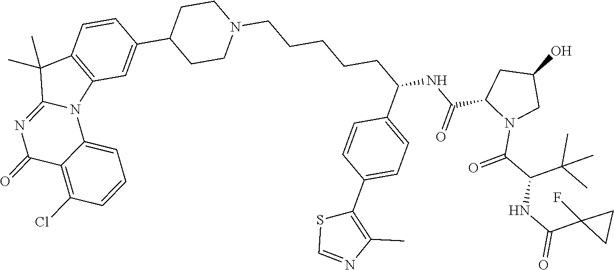 [SMILES: Cc1ncsc1-c1ccc([C@H](CCCCCN2CCC(c3ccc4c(c3)-n3c(nc(=O)c5c(Cl)cccc53)C4(C)C)CC2)NC(=O)[C@@H]2C[C@@H](O)CN2C(=O)[C@@H](NC(=O)C2(F)CC2)C(C)(C)C)cc1] | (2S,4R)-N-((S)-6-(4-(4-chloro-7,7- dimethyl-5-oxo-5,7-dihydroindolo[1,2- a]quinazolin-10-yl)piperidin-1-yl)-1-(4- (4-methylthiazol-5-yl)phenyl)hexyl)-1- ((S)-2-(1-fluorocyclopropane-1- carboxamido)-3,3-dimethylbutanoyl)-4- hydroxypyrrolidine-2-carboxamide |
| B22 | 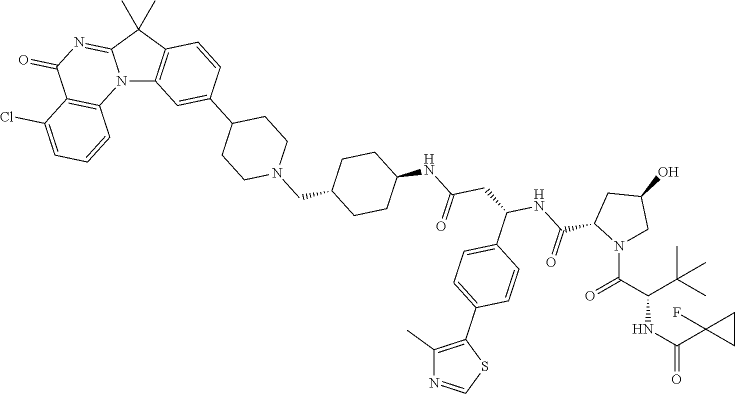 [SMILES: Cc1ncsc1-c1ccc([C@H](CC(=O)N[C@H]2CC[C@H](CN3CCC(c4ccc5c(c4)-n4c(nc(=O)c6c(Cl)cccc64)C5(C)C)CC3)CC2)NC(=O)[C@@H]2C[C@@H](O)CN2C(=O)[C@@H](NC(=O)C2(F)CC2)C(C)(C)C)cc1] | (2S,4R)-N-((S)-3-(((1r,4S)-4-((4-(4- chloro-7,7-dimethyl-5-oxo-5,7- dihydroindolo[1,2-a]quinazolin-10- yl)piperidin-1- yl)methyl)cyclohexyl)amino)-1-(4-(4- methylthiazol-5-yl)phenyl)-3-oxopropyl)- 1-((S)-2-(1-fluorocyclopropane-1- carboxamido)-3,3-dimethylbutanoyl)-4- hydroxypyrrolidine-2-carboxamide |
| B23 | 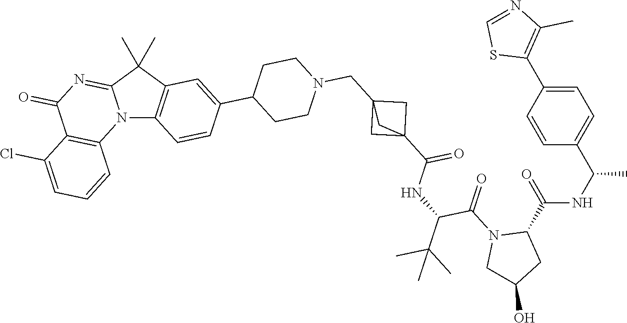 [SMILES: Cc1ncsc1-c1ccc([C@H](C)NC(=O)[C@@H]2C[C@@H](O)CN2C(=O)[C@@H](NC(=O)C23CC(CN4CCC(c5ccc6c(c5)C(C)(C)c5nc(=O)c7c(Cl)cccc7n5-6)CC4)(C2)C3)C(C)(C)C)cc1] | (2S,4R)-1-((S)-2-(3-((4-(4-chloro-7,7- dimethyl-5-oxo-5,7-dihydroindolo[1,2- a]quinazolin-9-yl)piperidin-1- yl)methyl)bicyclo[1.1.1]pentane-1- carboxamido)-3,3-dimethylbutanoyl)-4- hydroxy-N-((S)-1-(4-(4-methylthiazol-5- yl)phenyl)ethyl)pyrrolidine-2- carboxamide |
| B24 | 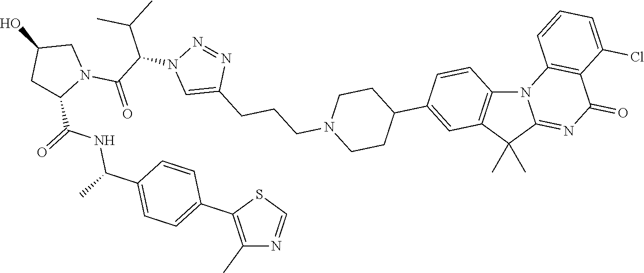 [SMILES: Cc1ncsc1-c1ccc([C@H](C)NC(=O)[C@@H]2C[C@@H](O)CN2C(=O)[C@H](C(C)C)n2cc(CCCN3CCC(c4ccc5c(c4)C(C)(C)c4nc(=O)c6c(Cl)cccc6n4-5)CC3)nn2)cc1] | (2S,4R)-1-((S)-2-(4-(3-(4-(4-chloro-7,7- dimethyl-5-oxo-5,7-dihydroindolo[1,2- a]quinazolin-9-yl)piperidin-1-yl)propyl)- 1H-1,2,3-triazol-1-yl)-3-methylbutanoyl)- 4-hydroxy-N-((S)-1-(4-(4-methylthiazol- 5-yl)phenyl)ethyl)pyrrolidine-2- carboxamide |
| B25 | 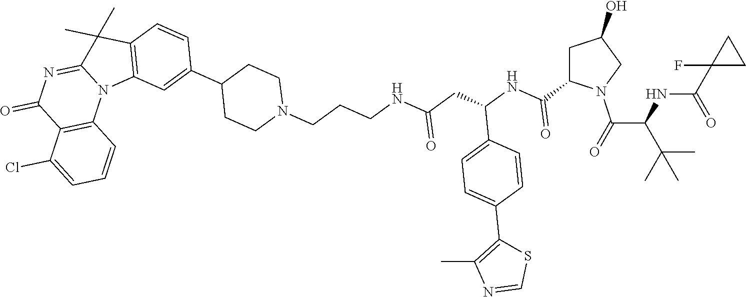 [SMILES: Cc1ncsc1-c1ccc([C@H](CC(=O)NCCCN2CCC(c3ccc4c(c3)-n3c(nc(=O)c5c(Cl)cccc53)C4(C)C)CC2)NC(=O)[C@@H]2C[C@@H](O)CN2C(=O)[C@@H](NC(=O)C2(F)CC2)C(C)(C)C)cc1] | (2S,4R)-N-((S)-3-((3-(4-(4-chloro-7,7- dimethyl-5-oxo-5,7-dihydroindolo[1,2- a]quinazolin-10-yl)piperidin-1- yl)propyl)amino)-1-(4-(4-methylthiazol- 5-yl)phenyl)-3-oxopropyl)-1-((S)-2-(1- fluorocyclopropane-1-carboxamido)-3,3- dimethylbutanoyl)-4-hydroxypyrrolidine- 2-carboxamide |
| B26 | 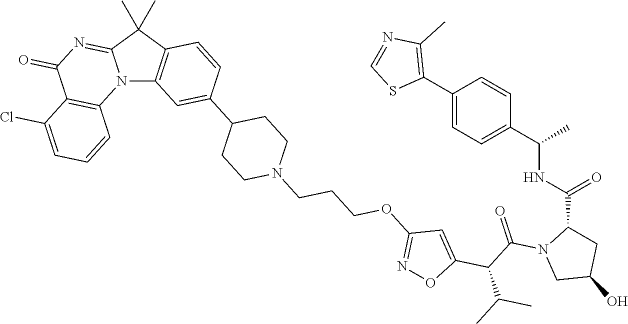 [SMILES: Cc1ncsc1-c1ccc([C@H](C)NC(=O)[C@@H]2C[C@@H](O)CN2C(=O)[C@@H](c2cc(OCCCN3CCC(c4ccc5c(c4)-n4c(nc(=O)c6c(Cl)cccc64)C5(C)C)CC3)no2)C(C)C)cc1] | (2S,4R)-1-((R)-2-(3-(3-(4-(4-chloro-7,7- dimethyl-5-oxo-5,7-dihydroindolo[1,2- a]quinazolin-10-yl)piperidin-1- yl)propoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B27 | 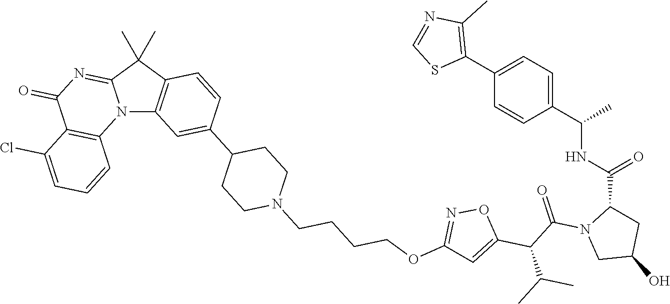 [SMILES: Cc1ncsc1-c1ccc([C@H](C)NC(=O)[C@@H]2C[C@@H](O)CN2C(=O)[C@@H](c2cc(OCCCCN3CCC(c4ccc5c(c4)-n4c(nc(=O)c6c(Cl)cccc64)C5(C)C)CC3)no2)C(C)C)cc1] | (2S,4R)-1-((R)-2-(3-(4-(4-(4-chloro-7,7- dimethyl-5-oxo-5,7-dihydroindolo[1,2- a]quinazolin-10-yl)piperidin-1- yl)butoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B28 | 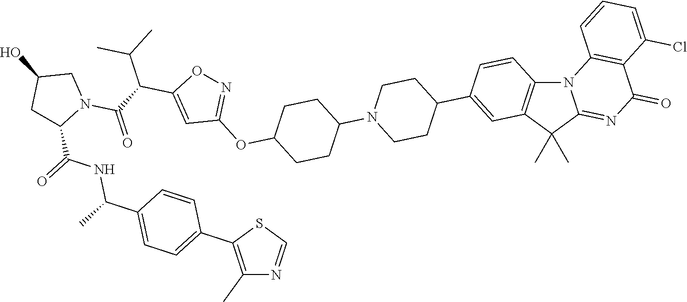 [SMILES: Cc1ncsc1-c1ccc([C@H](C)NC(=O)[C@@H]2C[C@@H](O)CN2C(=O)[C@@H](c2cc(OC3CCC(N4CCC(c5ccc6c(c5)C(C)(C)c5nc(=O)c7c(Cl)cccc7n5-6)CC4)CC3)no2)C(C)C)cc1] | (2S,4R)-1-((R)-2-(3-((4-(4-(4-chloro-7,7- dimethyl-5-oxo-5,7-dihydroindolo[1,2- a]quinazolin-9-yl)piperidin-1- yl)cyclohexyl)oxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B29 | 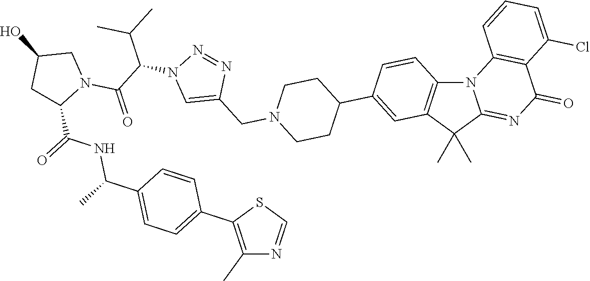 [SMILES: Cc1ncsc1-c1ccc([C@H](C)NC(=O)[C@@H]2C[C@@H](O)CN2C(=O)[C@H](C(C)C)n2cc(CN3CCC(c4ccc5c(c4)C(C)(C)c4nc(=O)c6c(Cl)cccc6n4-5)CC3)nn2)cc1] | (2S,4R)-1-((S)-2-(4-((4-(4-chloro-7,7- dimethyl-5-oxo-5,7-dihydroindolo[1,2- a]quinazolin-9-yl)piperidin-1-yl)methyl)- 1H-1,2,3-triazol-1-yl)-3-methylbutanoyl)- 4-hydroxy-N-((S)-1-(4-(4-methylthiazol- 5-yl)phenyl)ethyl)pyrrolidine-2- carboxamide |
| B30 | 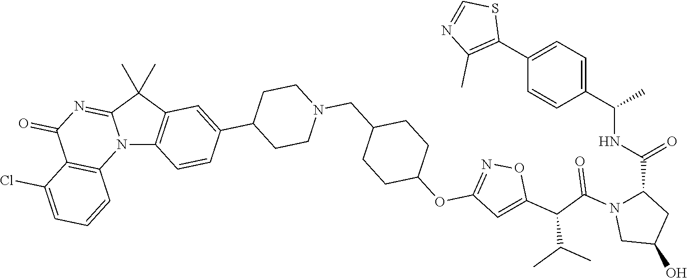 [SMILES: Cc1ncsc1-c1ccc([C@H](C)NC(=O)[C@@H]2C[C@@H](O)CN2C(=O)[C@@H](c2cc(OC3CCC(CN4CCC(c5ccc6c(c5)C(C)(C)c5nc(=O)c7c(Cl)cccc7n5-6)CC4)CC3)no2)C(C)C)cc1] | (2S,4R)-1-((R)-2-(3-((4-((4-(4-chloro- 7,7-dimethyl-5-oxo-5,7- dihydroindolo[1,2-a]quinazolin-9- yl)piperidin-1- yl)methyl)cyclohexyl)oxy)isoxazol-5-yl)- 3-methylbutanoyl)-4-hydroxy-N-((S)-1- (4-(4-methylthiazol-5- yl)phenyl)ethyl)pyrrolidine-2- carboxamide |
| B31 | 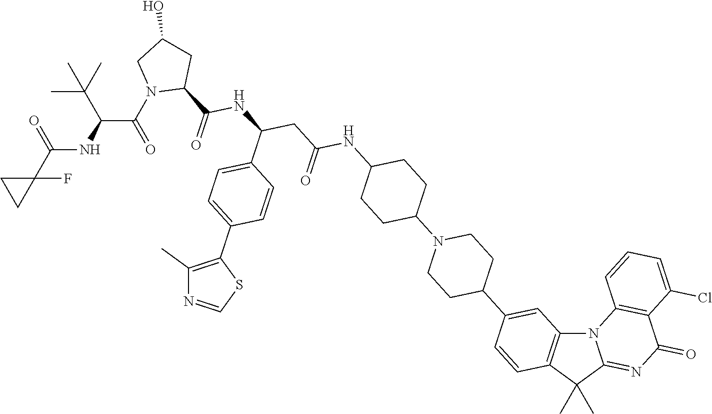 [SMILES: Cc1ncsc1-c1ccc([C@H](CC(=O)NC2CCC(N3CCC(c4ccc5c(c4)-n4c(nc(=O)c6c(Cl)cccc64)C5(C)C)CC3)CC2)NC(=O)[C@@H]2C[C@@H](O)CN2C(=O)[C@@H](NC(=O)C2(F)CC2)C(C)(C)C)cc1] | (2S,4R)-N-((S)-3-((4-(4-(4-chloro-7,7- dimethyl-5-oxo-5,7-dihydroindolo[1,2- a]quinazolin-10-yl)piperidin-1- yl)cyclohexyl)amino)-1-(4-(4- methylthiazol-5-yl)phenyl)-3-oxopropyl)- 1-((S)-2-(1-fluorocyclopropane-1- carboxamido)-3,3-dimethylbutanoyl)-4- hydroxypyrrolidine-2-carboxamide |
| B32 | 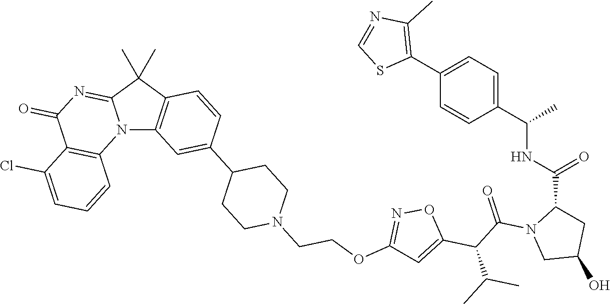 [SMILES: Cc1ncsc1-c1ccc([C@H](C)NC(=O)[C@@H]2C[C@@H](O)CN2C(=O)[C@@H](c2cc(OCCN3CCC(c4ccc5c(c4)-n4c(nc(=O)c6c(Cl)cccc64)C5(C)C)CC3)no2)C(C)C)cc1] | (2S,4R)-1-((R)-2-(3-(2-(4-(4-chloro-7,7- dimethyl-5-oxo-5,7-dihydroindolo[1,2- a]quinazolin-10-yl)piperidin-1- yl)ethoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B33 | 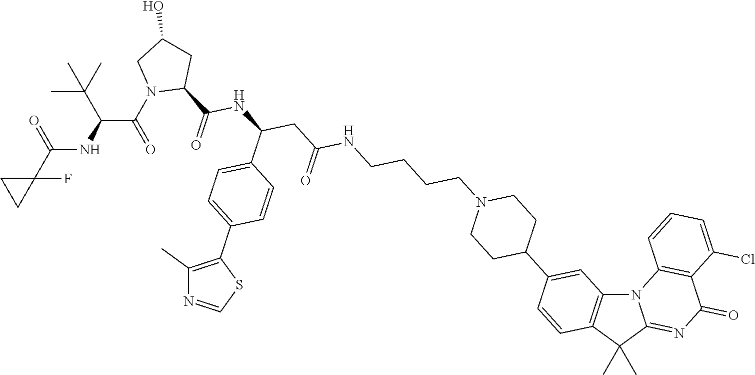 [SMILES: Cc1ncsc1-c1ccc([C@H](CC(=O)NCCCCN2CCC(c3ccc4c(c3)-n3c(nc(=O)c5c(Cl)cccc53)C4(C)C)CC2)NC(=O)[C@@H]2C[C@@H](O)CN2C(=O)[C@@H](NC(=O)C2(F)CC2)C(C)(C)C)cc1] | (2S,4R)-N-((S)-3-((4-(4-(4-chloro-7,7- dimethyl-5-oxo-5,7-dihydroindolo[1,2- a]quinazolin-10-yl)piperidin-1- yl)butyl)amino)-1-(4-(4-methylthiazol-5- yl)phenyl)-3-oxopropyl)-1-((S)-2-(1- fluorocyclopropane-1-carboxamido)-3,3- dimethylbutanoyl)-4-hydroxypyrrolidine- 2-carboxamide |
| B34 | 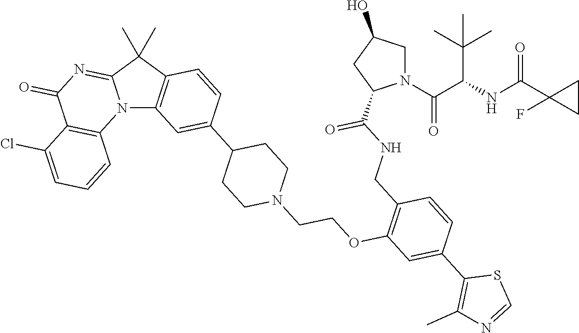 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCC(c3ccc4c(c3)-n3c(nc(=O)c5c(Cl)cccc53)C4(C)C)CC2)c1] | (2S,4R)-N-(2-(2-(4-(4-chloro-7,7- dimethyl-5-oxo-5,7-dihydroindolo[1,2- a]quinazolin-10-yl)piperidin-1- yl)ethoxy)-4-(4-methylthiazol-5- yl)benzyl)-1-((S)-2-(1- fluorocyclopropane-1-carboxamido)-3,3- dimethylbutanoyl)-4-hydroxypyrrolidine- 2-carboxamide |
| B35 | 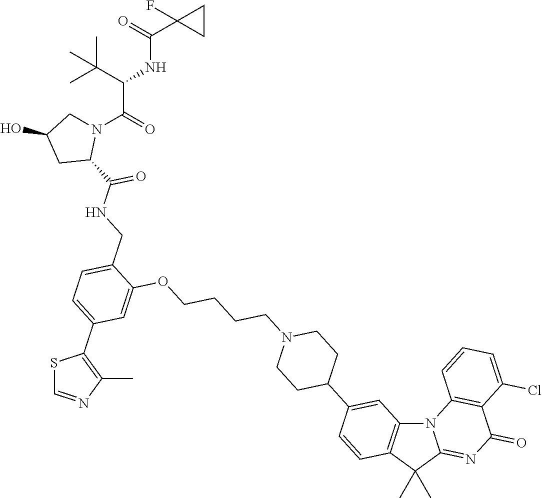 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCN2CCC(c3ccc4c(c3)-n3c(nc(=O)c5c(Cl)cccc53)C4(C)C)CC2)c1] | (2S,4R)-N-(2-(4-(4-(4-chloro-7,7- dimethyl-5-oxo-5,7-dihydroindolo[1,2- a]quinazolin-10-yl)piperidin-1- yl)butoxy)-4-(4-methylthiazol-5- yl)benzyl)-1-((S)-2-(1- fluorocyclopropane-1-carboxamido)-3,3- dimethylbutanoyl)-4-hydroxypyrrolidine- 2-carboxamide |
| B36 | 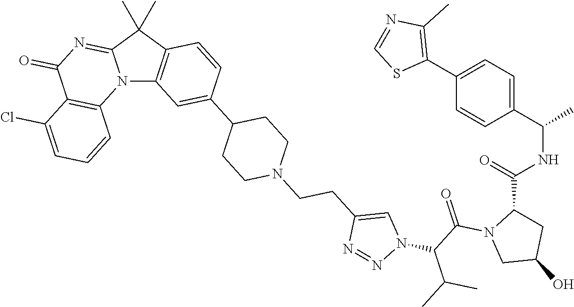 | (2S,4R)-1-((S)-2-(4-(2-(4-(4-chloro-7,7- dimethyl-5-oxo-5,7-dihydroindolo[1,2- a]quinazolin-10-yl)piperidin-1-yl)ethyl)- 1H-1,2,3-triazol-1-yl)-3-methylbutanoyl)- 4-hydroxy-N-((S)-1-(4-(4-methylthiazol- 5-yl)phenyl)ethyl)pyrrolidine-2- carboxamide |
| B37 | 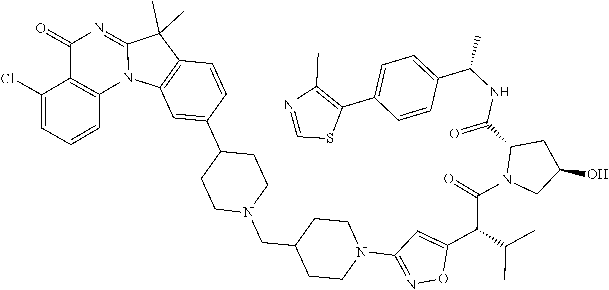 [SMILES: Cc1ncsc1-c1ccc([C@H](C)NC(=O)[C@@H]2C[C@@H](O)CN2C(=O)[C@@H](c2cc(N3CCC(CN4CCC(c5ccc6c(c5)-n5c(nc(=O)c7c(Cl)cccc75)C6(C)C)CC4)CC3)no2)C(C)C)cc1] | (2S,4R)-1-((R)-2-(3-(4-((4-(4-chloro-7,7- dimethyl-5-oxo-5,7-dihydroindolo[1,2- a]quinazolin-10-yl)piperidin-1- yl)methyl)piperidin-1-yl)isoxazol-5-yl)- 3-methylbutanoyl)-4-hydroxy-N-((S)-1- (4-(4-methylthiazol-5- yl)phenyl)ethyl)pyrrolidine-2- carboxamide |
| B38 | 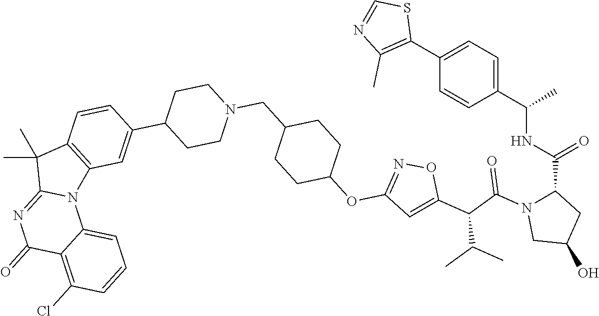 [SMILES: Cc1ncsc1-c1ccc([C@H](C)NC(=O)[C@@H]2C[C@@H](O)CN2C(=O)[C@@H](c2cc(OC3CCC(CN4CCC(c5ccc6c(c5)-n5c(nc(=O)c7c(Cl)cccc75)C6(C)C)CC4)CC3)no2)C(C)C)cc1] | (2S,4R)-1-((R)-2-(3-((4-((4-(4-chloro- 7,7-dimethyl-5-oxo-5,7- dihydroindolo[1,2-a]quinazolin-10- yl)piperidin-1- yl)methyl)cyclohexyl)oxy)isoxazol-5-yl)- 3-methylbutanoyl)-4-hydroxy-N-((S)-1- (4-(4-methylthiazol-5- yl)phenyl)ethyl)pyrrolidine-2- carboxamide |
| B39 | 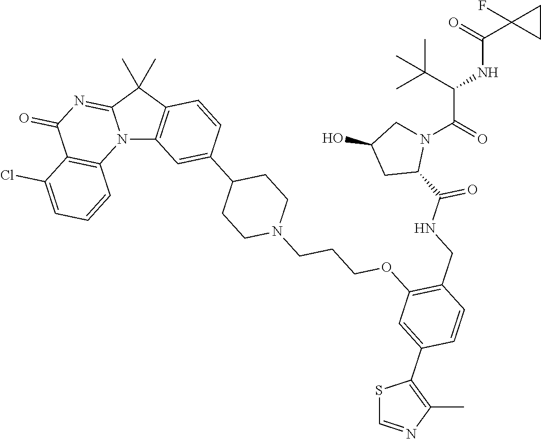 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCN2CCC(c3ccc4c(c3)-n3c(nc(=O)c5c(Cl)cccc53)C4(C)C)CC2)c1] | (2S,4R)-N-(2-(3-(4-(4-chloro-7,7- dimethyl-5-oxo-5,7-dihydroindolo[1,2- a]quinazolin-10-yl)piperidin-1- yl)propoxy)-4-(4-methylthiazol-5- yl)benzyl)-1-((S)-2-(1- fluorocyclopropane-1-carboxamido)-3,3- dimethylbutanoyl)-4-hydroxypyrrolidine- 2-carboxamide |
| B40 | 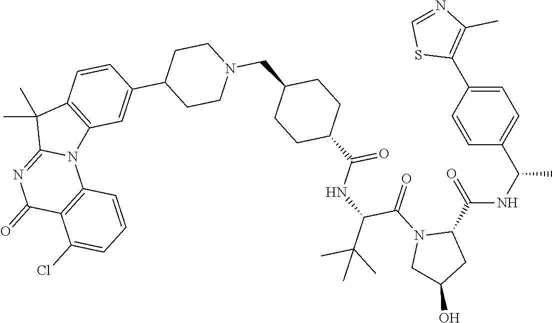 [SMILES: Cc1ncsc1-c1ccc([C@H](C)NC(=O)[C@@H]2C[C@@H](O)CN2C(=O)[C@@H](NC(=O)[C@H]2CC[C@H](CN3CCC(c4ccc5c(c4)-n4c(nc(=O)c6c(Cl)cccc64)C5(C)C)CC3)CC2)C(C)(C)C)cc1] | (2S,4R)-1-((S)-2-((1r,4S)-4-((4-(4- chloro-7,7-dimethyl-5-oxo-5,7- dihydroindolo[1,2-a]quinazolin-10- yl)piperidin-1-yl)methyl)cyclohexane-1- carboxamido)-3,3-dimethylbutanoyl)-4- hydroxy-N-((S)-1-(4-(4-methylthiazol-5- yl)phenyl)ethyl)pyrrolidine-2- carboxamide |
| B41 | 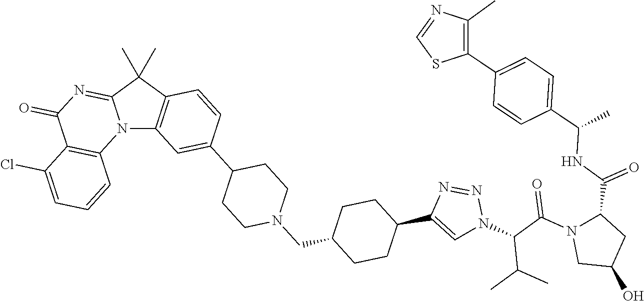 [SMILES: Cc1ncsc1-c1ccc([C@H](C)NC(=O)[C@@H]2C[C@@H](O)CN2C(=O)[C@H](C(C)C)n2cc([C@H]3CC[C@H](CN4CCC(c5ccc6c(c5)-n5c(nc(=O)c7c(Cl)cccc75)C6(C)C)CC4)CC3)nn2)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3-methylbutanoyl)-4- hydroxy-N-((S)-1-(4-(4-methylthiazol-5- yl)phenyl)ethyl)pyrrolidine-2- carboxamide |
| B42 | 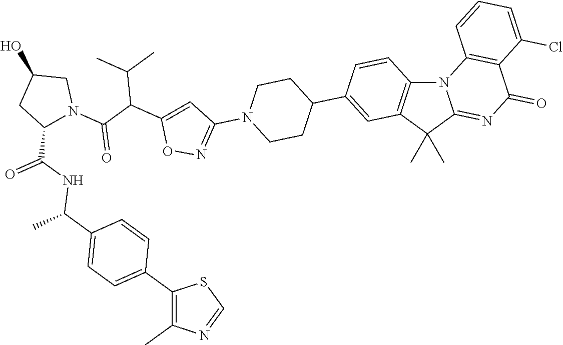 [SMILES: Cc1ncsc1-c1ccc([C@H](C)NC(=O)[C@@H]2C[C@@H](O)CN2C(=O)C(c2cc(N3CCC(c4ccc5c(c4)C(C)(C)c4nc(=O)c6c(Cl)cccc6n4-5)CC3)no2)C(C)C)cc1] | (2S,4R)-1-(2-(3-(4-(4-chloro-7,7- dimethyl-5-oxo-5,7-dihydroindolo[1,2- a]quinazolin-9-yl)piperidin-1-yl)isoxazol- 5-yl)-3-methylbutanoyl)-4-hydroxy-N- ((S)-1-(4-(4-methylthiazol-5- yl)phenyl)ethyl)pyrrolidine-2- carboxamide |
| B43 | 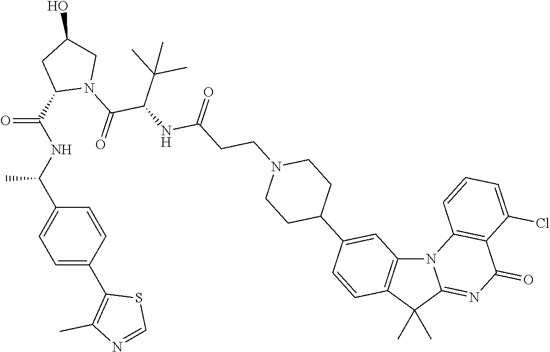 [SMILES: Cc1ncsc1-c1ccc([C@H](C)NC(=O)[C@@H]2C[C@@H](O)CN2C(=O)[C@@H](NC(=O)CCN2CCC(c3ccc4c(c3)-n3c(nc(=O)c5c(Cl)cccc53)C4(C)C)CC2)C(C)(C)C)cc1] | (2S,4R)-1-((S)-2-(3-(4-(4-chloro-7,7- dimethyl-5-oxo-5,7-dihydroindolo[1,2- a]quinazolin-10-yl)piperidin-1- yl)propanamido)-3,3-dimethylbutanoyl)- 4-hydroxy-N-((S)-1-(4-(4-methylthiazol- 5-yl)phenyl)ethyl)pyrrolidine-2- carboxamide |
| B44 | 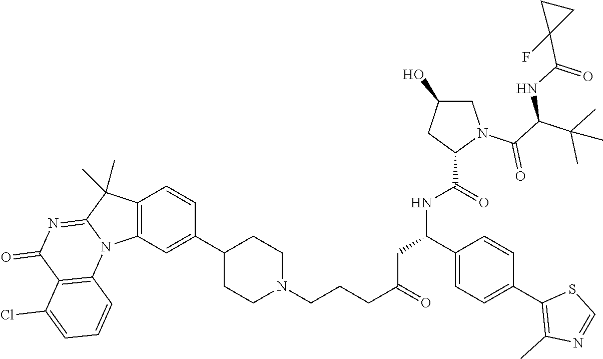 [SMILES: Cc1ncsc1-c1ccc([C@H](CC(=O)CCCN2CCC(c3ccc4c(c3)-n3c(nc(=O)c5c(Cl)cccc53)C4(C)C)CC2)NC(=O)[C@@H]2C[C@@H](O)CN2C(=O)[C@@H](NC(=O)C2(F)CC2)C(C)(C)C)cc1] | (2S,4R)-N-((S)-3-((2-(4-(4-chloro-7,7- dimethyl-5-oxo-5,7-dihydroindolo[1,2- a]quinazolin-10-yl)piperidin-1- yl)ethyl)amino)-1-(4-(4-methylthiazol-5- yl)phenyl)-3-oxopropyl)-1-((S)-2-(1- fluorocyclopropane-1-carboxamido)-3,3- dimethylbutanoyl)-4-hydroxypyrrolidine- 2-carboxamide |
| B45 | 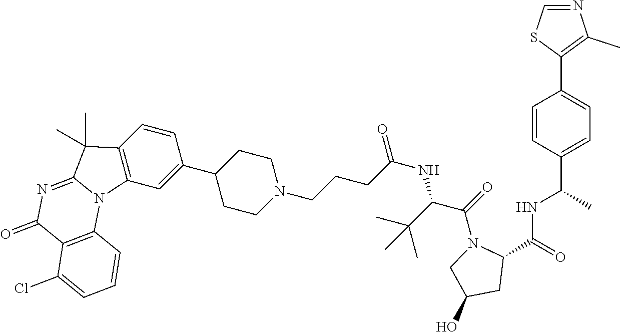 [SMILES: Cc1ncsc1-c1ccc([C@H](C)NC(=O)[C@@H]2C[C@@H](O)CN2C(=O)[C@@H](NC(=O)CCCN2CCC(c3ccc4c(c3)-n3c(nc(=O)c5c(Cl)cccc53)C4(C)C)CC2)C(C)(C)C)cc1] | (2S,4R)-1-((S)-2-(4-(4-(4-chloro-7,7- dimethyl-5-oxo-5,7-dihydroindolo[1,2- a]quinazolin-10-yl)piperidin-1- yl)butanamido)-3,3-dimethylbutanoyl)-4- hydroxy-N-((S)-1-(4-(4-methylthiazol-5- yl)phenyl)ethyl)pyrrolidine-2- carboxamide |
| B46 | 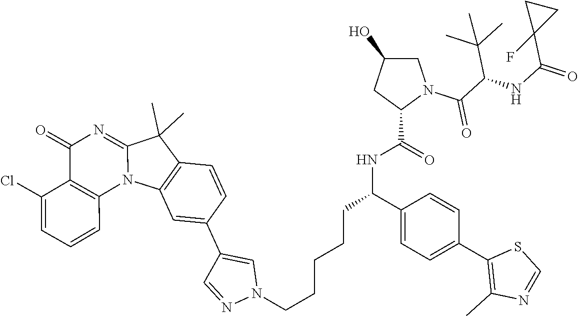 [SMILES: Cc1ncsc1-c1ccc([C@H](CCCCCn2cc(-c3ccc4c(c3)-n3c(nc(=O)c5c(Cl)cccc53)C4(C)C)cn2)NC(=O)[C@@H]2C[C@@H](O)CN2C(=O)[C@@H](NC(=O)C2(F)CC2)C(C)(C)C)cc1] | (2S,4R)-N-((S)-6-(4-(4-chloro-7,7- dimethyl-5-oxo-5,7-dihydroindolo[1,2- a]quinazolin-10-yl)-1H-pyrazol-1-yl)-1- (4-(4-methylthiazol-5-yl)phenyl)hexyl)- 1-((S)-2-(1-fluorocyclopropane-1- carboxamido)-3,3-dimethylbutanoyl)-4- hydroxypyrrolidine-2-carboxamide |
| B47 | 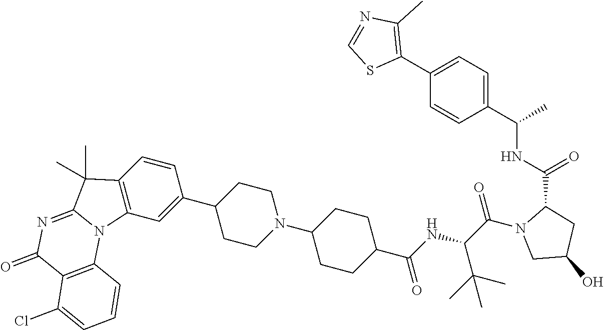 [SMILES: Cc1ncsc1-c1ccc([C@H](C)NC(=O)[C@@H]2C[C@@H](O)CN2C(=O)[C@@H](NC(=O)C2CCC(N3CCC(c4ccc5c(c4)-n4c(nc(=O)c6c(Cl)cccc64)C5(C)C)CC3)CC2)C(C)(C)C)cc1] | (2S,4R)-1-((S)-2-(4-(4-(4-chloro-7,7- dimethyl-5-oxo-5,7-dihydroindolo[1,2- a]quinazolin-10-yl)piperidin-1- yl)cyclohexane-1-carboxamido)-3,3- dimethylbutanoyl)-4-hydroxy-N-((S)-1- (4-(4-methylthiazol-5- yl)phenyl)ethyl)pyrrolidine-2- carboxamide |
| B48 | 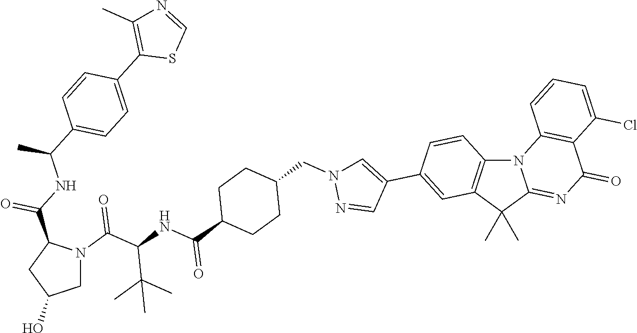 [SMILES: Cc1ncsc1-c1ccc([C@H](C)NC(=O)[C@@H]2C[C@@H](O)CN2C(=O)[C@@H](NC(=O)[C@H]2CC[C@H](Cn3cc(-c4ccc5c(c4)C(C)(C)c4nc(=O)c6c(Cl)cccc6n4-5)cn3)CC2)C(C)(C)C)cc1] | (2S,4R)-1-((S)-2-((1r,4S)-4-((4-(4- chloro-7,7-dimethyl-5-oxo-5,7- dihydroindolo[1,2-a]quinazolin-9-yl)-1H- pyrazol-1-yl)methyl)cyclohexane-1- carboxamido)-3,3-dimethylbutanoyl)-4- hydroxy-N-((S)-1-(4-(4-methylthiazol-5- yl)phenyl)ethyl)pyrrolidine-2- carboxamide |
| B49 | 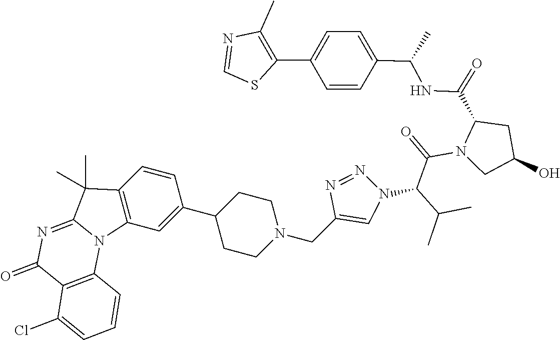 [SMILES: Cc1ncsc1-c1ccc([C@H](C)NC(=O)[C@@H]2C[C@@H](O)CN2C(=O)[C@H](C(C)C)n2cc(CN3CCC(c4ccc5c(c4)-n4c(nc(=O)c6c(Cl)cccc64)C5(C)C)CC3)nn2)cc1] | (2S,4R)-1-((S)-2-(4-((4-(4-chloro-7,7- dimethyl-5-oxo-5,7-dihydroindolo[1,2- a]quinazolin-10-yl)piperidin-1- yl)methyl)-1H-1,2,3-triazol-1-yl)-3- methylbutanoyl)-4-hydroxy-N-((S)-1-(4- (4-methylthiazol-5- yl)phenyl)ethyl)pyrrolidine-2- carboxamide |
| B50 | 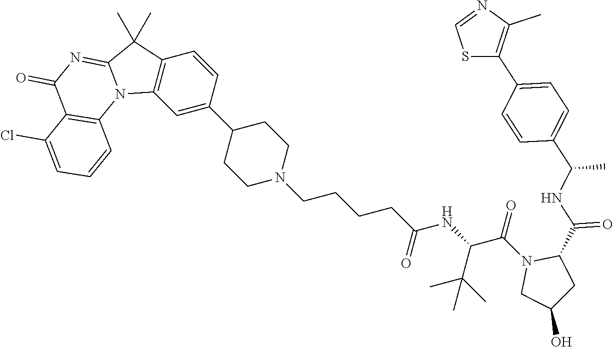 [SMILES: Cc1ncsc1-c1ccc([C@H](C)NC(=O)[C@@H]2C[C@@H](O)CN2C(=O)[C@@H](NC(=O)CCCCN2CCC(c3ccc4c(c3)-n3c(nc(=O)c5c(Cl)cccc53)C4(C)C)CC2)C(C)(C)C)cc1] | (2S,4R)-1-((S)-2-(5-(4-(4-chloro-7,7- dimethyl-5-oxo-5,7-dihydroindolo[1,2- a]quinazolin-10-yl)piperidin-1- yl)pentanamido)-3,3-dimethylbutanoyl)- 4-hydroxy-N-((S)-1-(4-(4-methylthiazol- 5-yl)phenyl)ethyl)pyrrolidine-2- carboxamide |
| B51 | 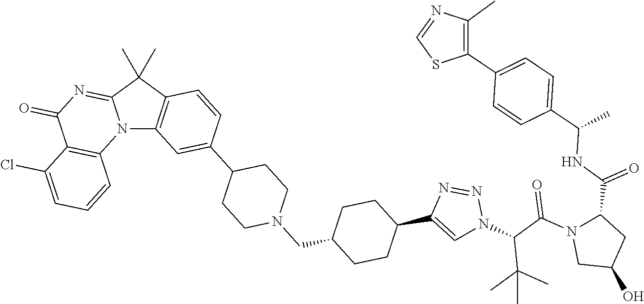 [SMILES: Cc1ncsc1-c1ccc([C@H](C)NC(=O)[C@@H]2C[C@@H](O)CN2C(=O)[C@@H](n2cc([C@H]3CC[C@H](CN4CCC(c5ccc6c(c5)-n5c(nc(=O)c7c(Cl)cccc75)C6(C)C)CC4)CC3)nn2)C(C)(C)C)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- 4-hydroxy-N-((S)-1-(4-(4-methylthiazol- 5-yl)phenyl)ethyl)pyrrolidine-2- carboxamide |
| B52 | 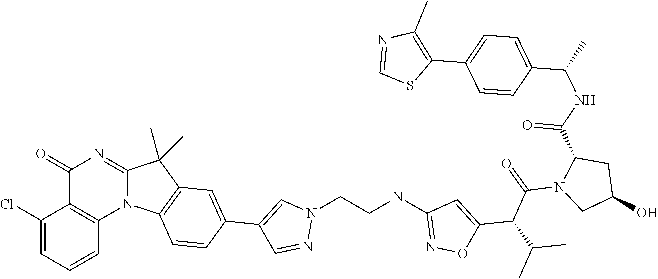 [SMILES: Cc1ncsc1-c1ccc([C@H](C)NC(=O)[C@@H]2C[C@@H](O)CN2C(=O)[C@@H](c2cc(NCCn3cc(-c4ccc5c(c4)C(C)(C)c4nc(=O)c6c(Cl)cccc6n4-5)cn3)no2)C(C)C)cc1] | (2S,4R)-1-((R)-2-(3-(2-(4-(4-chloro-7,7- dimethyl-5-oxo-5,7-dihydroindolo[1,2- a]quinazolin-9-yl)-1H-pyrazol-1- yl)ethoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B53 | 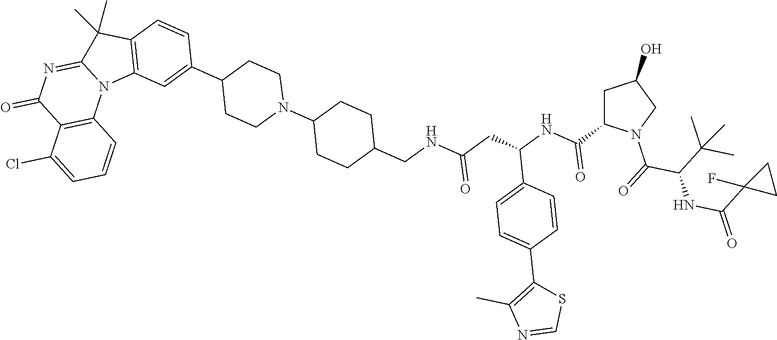 [SMILES: Cc1ncsc1-c1ccc([C@H](CC(=O)NCC2CCC(N3CCC(c4ccc5c(c4)-n4c(nc(=O)c6c(Cl)cccc64)C5(C)C)CC3)CC2)NC(=O)[C@@H]2C[C@@H](O)CN2C(=O)[C@@H](NC(=O)C2(F)CC2)C(C)(C)C)cc1] | (2S,4R)-N-((S)-3-(((4-(4-(4-chloro-7,7- dimethyl-5-oxo-5,7-dihydroindolo[1,2- a]quinazolin-10-yl)piperidin-1- yl)cyclohexyl)methyl)amino)-1-(4-(4- methylthiazol-5-yl)phenyl)-3-oxopropyl)- 1-((S)-2-(1-fluorocyclopropane-1- carboxamido)-3,3-dimethylbutanoyl)-4- hydroxypyrrolidine-2-carboxamide |
| B54 | 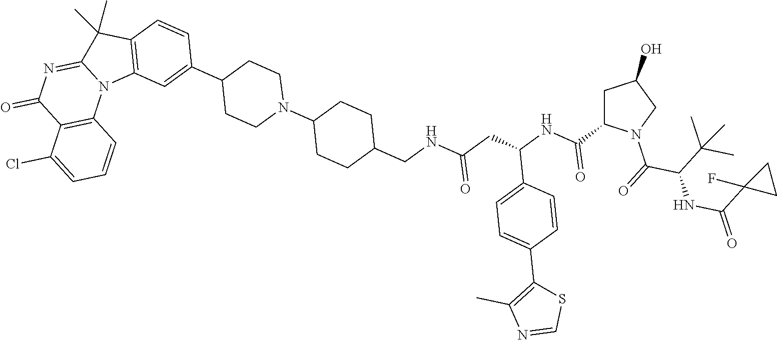 [SMILES: Cc1ncsc1-c1ccc([C@H](CC(=O)NCC2CCC(N3CCC(c4ccc5c(c4)-n4c(nc(=O)c6c(Cl)cccc64)C5(C)C)CC3)CC2)NC(=O)[C@@H]2C[C@@H](O)CN2C(=O)[C@@H](NC(=O)C2(F)CC2)C(C)(C)C)cc1] | (2S,4R)-N-((S)-3-(((4-(4-(4-chloro-7,7- dimethyl-5-oxo-5,7-dihydroindolo[1,2- a]quinazolin-10-yl)piperidin-1- yl)cyclohexyl)methyl)amino)-1-(4-(4- methylthiazol-5-yl)phenyl)-3-oxopropyl)- 1-((S)-2-(1-fluorocyclopropane-1- carboxamido)-3,3-dimethylbutanoyl)-4- hydroxypyrrolidine-2-carboxamide |
| B55 | 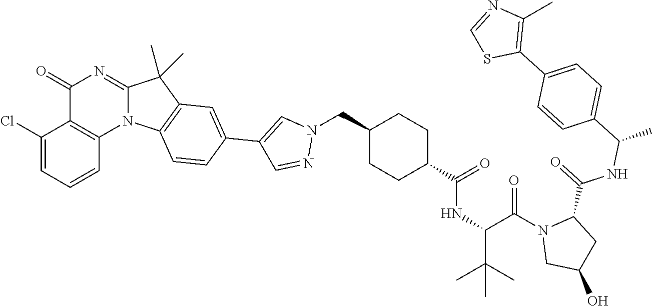 [SMILES: Cc1ncsc1-c1ccc([C@H](C)NC(=O)[C@@H]2C[C@@H](O)CN2C(=O)[C@@H](NC(=O)[C@H]2CC[C@H](Cn3cc(-c4ccc5c(c4)C(C)(C)c4nc(=O)c6c(Cl)cccc6n4-5)cn3)CC2)C(C)(C)C)cc1] | (2S,4R)-1-((S)-2-((11,4S)-4-((4-(4- chloro-7,7-dimethyl-5-oxo-5,7- dihydroindolo[1,2-a]quinazolin-10-yl)- 1H-pyrazol-1-yl)methyl)cyclohexane-1- carboxamido)-3,3-dimethylbutanoyl)-4- hydroxy-N-((S)-1-(4-(4-methylthiazol-5- yl)phenyl)ethyl)pyrrolidine-2- carboxamide |
| B56 | 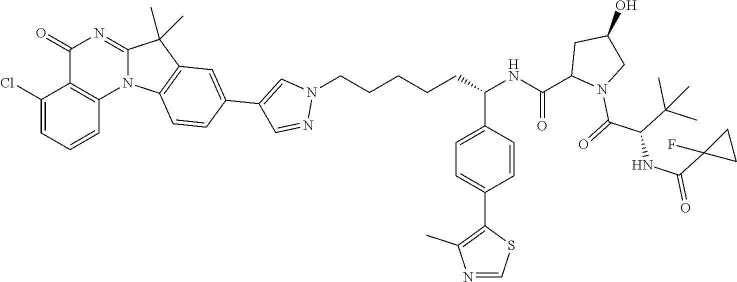 [SMILES: Cc1ncsc1-c1ccc([C@H](CCCCCn2cc(-c3ccc4c(c3)C(C)(C)c3nc(=O)c5c(Cl)cccc5n3-4)cn2)NC(=O)C2C[C@@H](O)CN2C(=O)[C@@H](NC(=O)C2(F)CC2)C(C)(C)C)cc1] | (2S,4R)-N-((S)-6-(4-(4-chloro-7,7- dimethyl-5-oxo-5,7-dihydroindolo[1,2- a]quinazolin-9-yl)-1H-pyrazol-1-yl)-1-(4- (4-methylthiazol-5-yl)phenyl)hexyl)-1- ((S)-2-(1-fluorocyclopropane-1- carboxamido)-3,3-dimethylbutanoyl)-4- hydroxypyrrolidine-2-carboxamide |
| B57 | 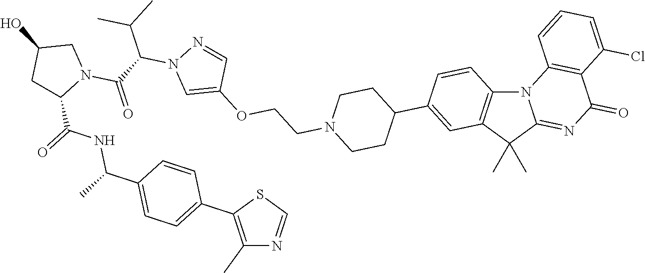 [SMILES: Cc1ncsc1-c1ccc([C@H](C)NC(=O)[C@@H]2C[C@@H](O)CN2C(=O)[C@H](C(C)C)n2cc(OCCN3CCC(c4ccc5c(c4)C(C)(C)c4nc(=O)c6c(Cl)cccc6n4-5)CC3)cn2)cc1] | (2S,4R)-1-(2-(4-(2-(4-(4-chloro-7,7- dimethyl-5-oxo-5,7-dihydroindolo[1,2- a]quinazolin-9-yl)piperidin-1-yl)ethoxy)- 1H-pyrazol-1-yl)-3-methylbutanoyl)-4- hydroxy-N-((S)-1-(4-(4-methylthiazol-5- yl)phenyl)ethyl)pyrrolidine-2- carboxamide |
| B58 | 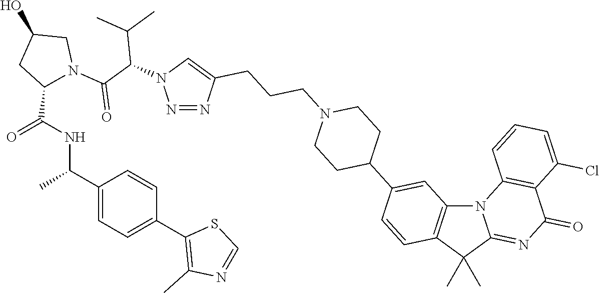 [SMILES: Cc1ncsc1-c1ccc([C@H](C)NC(=O)[C@@H]2C[C@@H](O)CN2C(=O)[C@H](C(C)C)n2cc(CCCN3CCC(c4ccc5c(c4)-n4c(nc(=O)c6c(Cl)cccc64)C5(C)C)CC3)nn2)cc1] | (2S,4R)-1-((S)-2-(4-(3-(4-(4-chloro-7,7- dimethyl-5-oxo-5,7-dihydroindolo[1,2- a]quinazolin-10-yl)piperidin-1- yl)propyl)-1H-1,2,3-triazol-1-yl)-3- methylbutanoyl)-4-hydroxy-N-((S)-1-(4- (4-methylthiazol-5- yl)phenyl)ethyl)pyrrolidine-2- carboxamide |
| B59 | 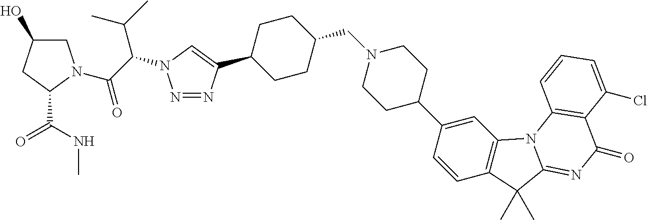 [SMILES: CNC(=O)[C@@H]1C[C@@H](O)CN1C(=O)[C@H](C(C)C)n1cc([C@H]2CC[C@H](CN3CCC(c4ccc5c(c4)-n4c(nc(=O)c6c(Cl)cccc64)C5(C)C)CC3)CC2)nn1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3-methylbutanoyl)-4- hydroxy-N-methylpyrrolidine-2- carboxamide |
| B60 | 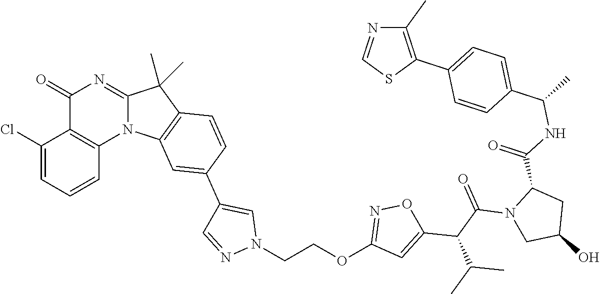 [SMILES: Cc1ncsc1-c1ccc([C@H](C)NC(=O)[C@@H]2C[C@@H](O)CN2C(=O)[C@@H](c2cc(OCCn3cc(-c4ccc5c(c4)-n4c(nc(=O)c6c(Cl)cccc64)C5(C)C)cn3)no2)C(C)C)cc1] | (2S,4R)-1-(2-(3-(2-(4-(4-chloro-7,7- dimethyl-5-oxo-5,7-dihydroindolo[1,2- a]quinazolin-10-yl)-1H-pyrazol-1- yl)ethoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B61 | 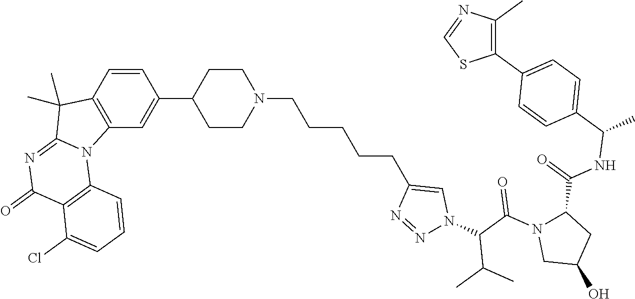 | (2S,4R)-1-((S)-2-(4-(5-(4-(4-chloro-7,7- dimethyl-5-oxo-5,7-dihydroindolo[1,2- a]quinazolin-10-yl)piperidin-1-yl)pentyl)- 1H-1,2,3-triazol-1-yl)-3-methylbutanoyl)- 4-hydroxy-N-((S)-1-(4-(4-methylthiazol- 5-yl)phenyl)ethyl)pyrrolidine-2- carboxamide |
| B62 | 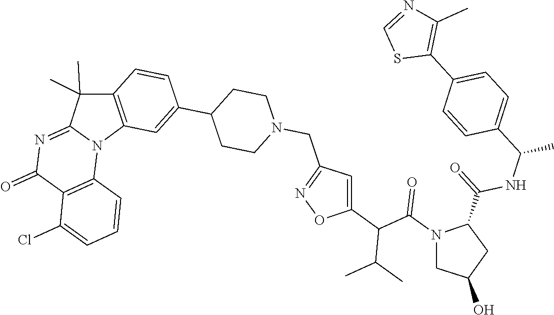 [SMILES: Cc1ncsc1-c1ccc([C@H](C)NC(=O)[C@@H]2C[C@@H](O)CN2C(=O)C(c2cc(CN3CCC(c4ccc5c(c4)-n4c(nc(=O)c6c(Cl)cccc64)C5(C)C)CC3)no2)C(C)C)cc1] | (2S,4R)-1-(2-(3-((4-(4-chloro-7,7- dimethyl-5-oxo-5,7-dihydroindolo[1,2- a]quinazolin-10-yl)piperidin-1- yl)methyl)isoxazol-5-yl)-3- methylbutanoyl)-4-hydroxy-N-((S)-1-(4- (4-methylthiazol-5- yl)phenyl)ethyl)pyrrolidine-2- carboxamide |
| B63 | 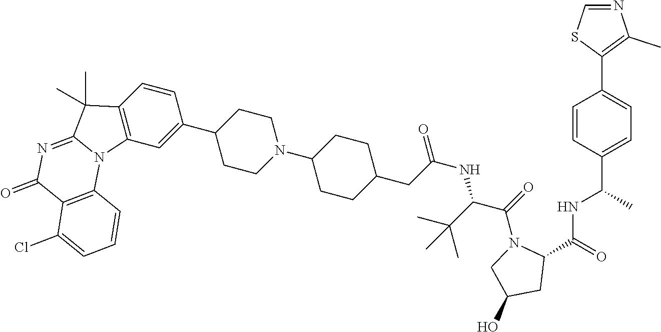 [SMILES: Cc1ncsc1-c1ccc([C@H](C)NC(=O)[C@@H]2C[C@@H](O)CN2C(=O)[C@@H](NC(=O)CC2CCC(N3CCC(c4ccc5c(c4)-n4c(nc(=O)c6c(Cl)cccc64)C5(C)C)CC3)CC2)C(C)(C)C)cc1] | (2S,4R)-1-((S)-2-(2-(4-(4-(4-chloro-7,7- dimethyl-5-oxo-5,7-dihydroindolo[1,2- a]quinazolin-10-yl)piperidin-1- yl)cyclohexyl)acetamido)-3,3- dimethylbutanoyl)-4-hydroxy-N-((S)-1- (4-(4-methylthiazol-5- yl)phenyl)ethyl)pyrrolidine-2- carboxamide |
| B64 | 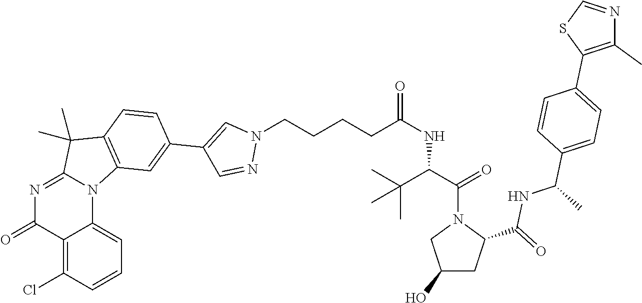 [SMILES: Cc1ncsc1-c1ccc([C@H](C)NC(=O)[C@@H]2C[C@@H](O)CN2C(=O)[C@@H](NC(=O)CCCCn2cc(-c3ccc4c(c3)-n3c(nc(=O)c5c(Cl)cccc53)C4(C)C)cn2)C(C)(C)C)cc1] | (2S,4R)-1-((S)-2-(5-(4-(4-chloro-7,7- dimethyl-5-oxo-5,7-dihydroindolo[1,2- a]quinazolin-10-yl)-1H-pyrazol-1- yl)pentanamido)-3,3-dimethylbutanoyl)- 4-hydroxy-N-((S)-1-(4-(4-methylthiazol- 5-yl)phenyl)ethyl)pyrrolidine-2- carboxamide |
| B65 | 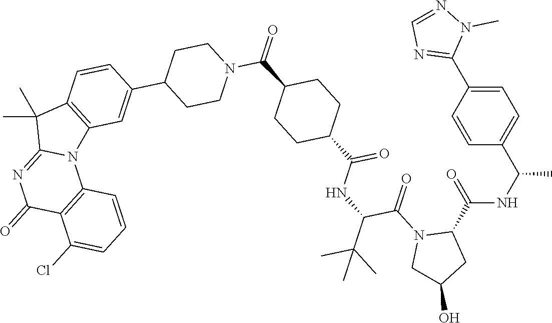 [SMILES: C[C@H](NC(=O)[C@@H]1C[C@@H](O)CN1C(=O)[C@@H](NC(=O)[C@H]1CC[C@H](C(=O)N2CCC(c3ccc4c(c3)-n3c(nc(=O)c5c(Cl)cccc53)C4(C)C)CC2)CC1)C(C)(C)C)c1ccc(-c2ncnn2C)cc1] | (2S,4R)-1-((S)-2-((1r,4S)-4-(4-(4-chloro- 7,7-dimethyl-5-oxo-5,7- dihydroindolo[1,2-a]quinazolin-10- yl)piperidine-1-carbonyl)cyclohexane-1- carboxamido)-3,3-dimethylbutanoyl)-4- hydroxy-N-((S)-1-(4-(1-methyl-1H-1,2,4- triazol-5-yl)phenyl)ethyl)pyrrolidine-2- carboxamide |
| B66 | 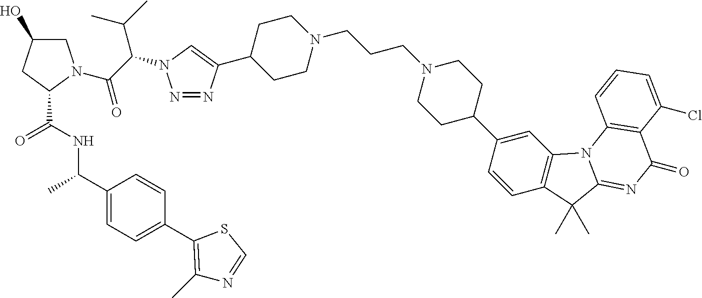 [SMILES: Cc1ncsc1-c1ccc([C@H](C)NC(=O)[C@@H]2C[C@@H](O)CN2C(=O)[C@H](C(C)C)n2cc(C3CCN(CCCN4CCC(c5ccc6c(c5)-n5c(nc(=O)c7c(Cl)cccc75)C6(C)C)CC4)CC3)nn2)cc1] | (2S,4R)-1-((S)-2-(4-(1-(3-(4-(4-chloro- 7,7-dimethyl-5-oxo-5,7- dihydroindolo[1,2-a]quinazolin-10- yl)piperidin-1-yl)propyl)piperidin-4-yl)- 1H-1,2,3-triazol-1-yl)-3-methylbutanoyl)- 4-hydroxy-N-((S)-1-(4-(4-methylthiazol- 5-yl)phenyl)ethyl)pyrrolidine-2- carboxamide |
| B67 | 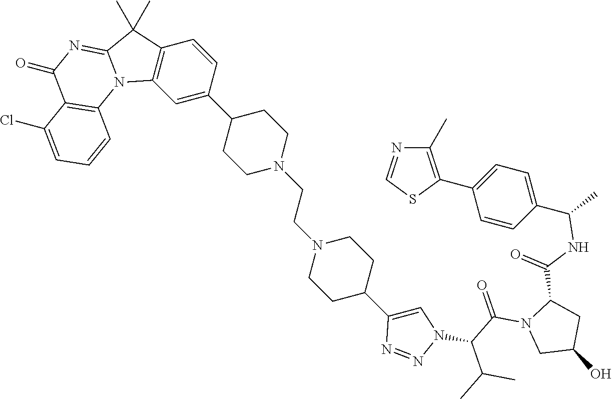 [SMILES: Cc1ncsc1-c1ccc([C@H](C)NC(=O)[C@@H]2C[C@@H](O)CN2C(=O)[C@H](C(C)C)n2cc(C3CCN(CCN4CCC(c5ccc6c(c5)-n5c(nc(=O)c7c(Cl)cccc75)C6(C)C)CC4)CC3)nn2)cc1] | (2S,4R)-1-((S)-2-(4-(1-(2-(4-(4-chloro- 7,7-dimethyl-5-oxo-5,7- dihydroindolo[1,2-a]quinazolin-10- yl)piperidin-1-yl)ethyl)piperidin-4-yl)- 1H-1,2,3-triazol-1-yl)-3-methylbutanoyl)- 4-hydroxy-N-((S)-1-(4-(4-methylthiazol- 5-yl)phenyl)ethyl)pyrrolidine-2- carboxamide |
| B68 | 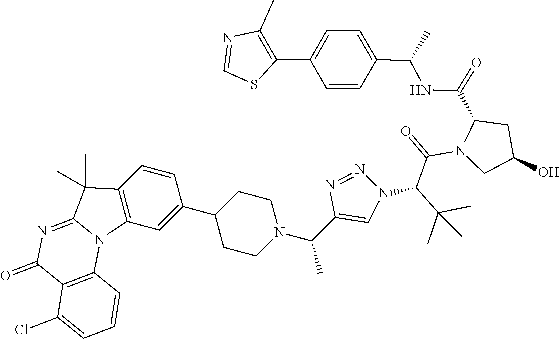 [SMILES: Cc1ncsc1-c1ccc([C@H](C)NC(=O)[C@@H]2C[C@@H](O)CN2C(=O)[C@@H](n2cc([C@H](C)N3CCC(c4ccc5c(c4)-n4c(nc(=O)c6c(Cl)cccc64)C5(C)C)CC3)nn2)C(C)(C)C)cc1] | (2S,4R)-1-((S)-2-(4-((S)-1-(4-(4-chloro- 7,7-dimethyl-5-oxo-5,7- dihydroindolo[1,2-a]quinazolin-10- yl)piperidin-1-yl)ethyl)-1H-1,2,3-triazol- 1-yl)-3,3-dimethylbutanoyl)-4-hydroxy- N-((S)-1-(4-(4-methylthiazol-5- yl)phenyl)ethyl)pyrrolidine-2- carboxamide |
| B69 | 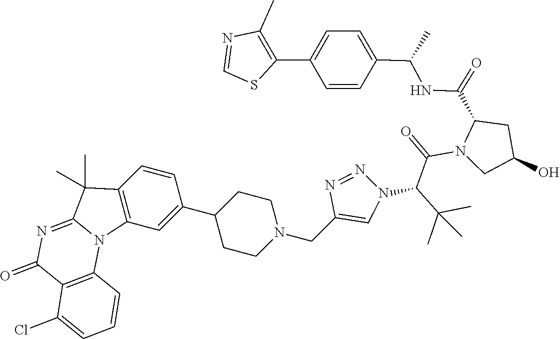 [SMILES: Cc1ncsc1-c1ccc([C@H](C)NC(=O)[C@@H]2C[C@@H](O)CN2C(=O)[C@@H](n2cc(CN3CCC(c4ccc5c(c4)-n4c(nc(=O)c6c(Cl)cccc64)C5(C)C)CC3)nn2)C(C)(C)C)cc1] | (2S,4R)-1-((S)-2-(4-((4-(4-chloro-7,7- dimethyl-5-oxo-5,7-dihydroindolo[1,2- a]quinazolin-10-yl)piperidin-1- yl)methyl)-1H-1,2,3-triazol-1-yl)-3,3- dimethylbutanoyl)-4-hydroxy-N-((S)-1- (4-(4-methylthiazol-5- yl)phenyl)ethyl)pyrrolidine-2- carboxamide |
| B70 | 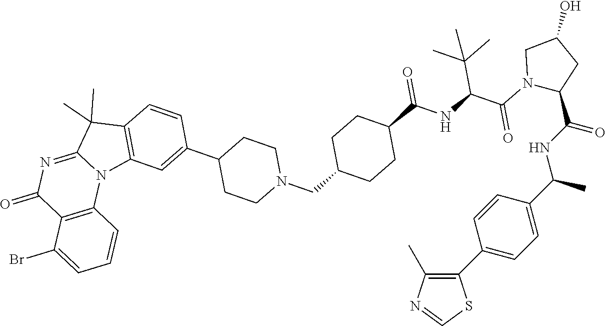 [SMILES: Cc1ncsc1-c1ccc([C@H](C)NC(=O)[C@@H]2C[C@@H](O)CN2C(=O)[C@@H](NC(=O)[C@H]2CC[C@H](CN3CCC(c4ccc5c(c4)-n4c(nc(=O)c6c(Br)cccc64)C5(C)C)CC3)CC2)C(C)(C)C)cc1] | (2S,4R)-1-((S)-2-((11,4S)-4-((4-(4- bromo-7,7-dimethyl-5-oxo-5,7- dihydroindolo[1,2-a]quinazolin-10- yl)piperidin-1-yl)methyl)cyclohexane-1- carboxamido)-3,3-dimethylbutanoyl)-4- hydroxy-N-((S)-1-(4-(4-methylthiazol-5- yl)phenyl)ethyl)pyrrolidine-2- carboxamide |
| B71 | 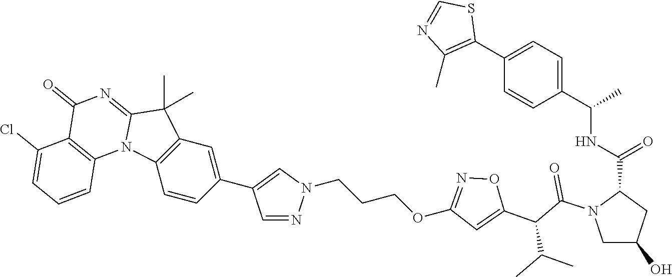 [SMILES: Cc1ncsc1-c1ccc([C@H](C)NC(=O)[C@@H]2C[C@@H](O)CN2C(=O)[C@@H](c2cc(OCCCn3cc(-c4ccc5c(c4)C(C)(C)c4nc(=O)c6c(Cl)cccc6n4-5)cn3)no2)C(C)C)cc1] | (2S,4R)-1-((R)-2-(3-(3-(4-(4-chloro-7,7- dimethyl-5-oxo-5,7-dihydroindolo[1,2- a]quinazolin-9-yl)-1H-pyrazol-1- yl)propoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B72 | 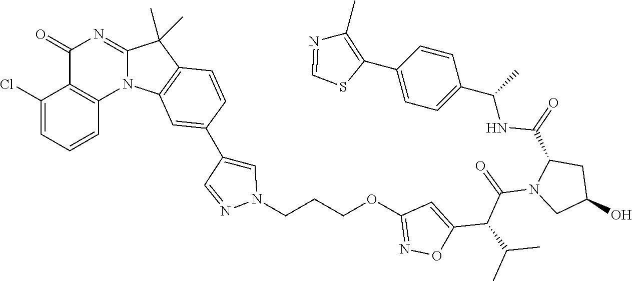 [SMILES: Cc1ncsc1-c1ccc([C@H](C)NC(=O)[C@@H]2C[C@@H](O)CN2C(=O)[C@@H](c2cc(OCCCn3cc(-c4ccc5c(c4)-n4c(nc(=O)c6c(Cl)cccc64)C5(C)C)cn3)no2)C(C)C)cc1] | (2S,4R)-1-((R)-2-(3-(3-(4-(4-chloro-7,7- dimethyl-5-oxo-5,7-dihydroindolo[1,2- a]quinazolin-10-yl)-1H-pyrazol-1- yl)propoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B73 | 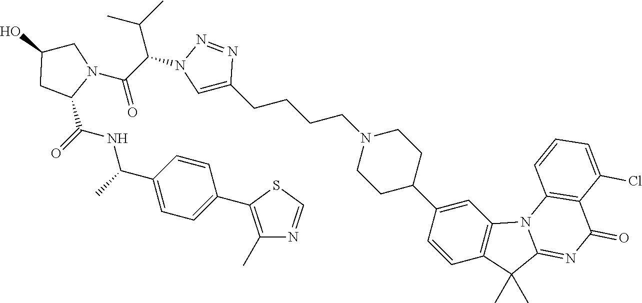 [SMILES: Cc1ncsc1-c1ccc([C@H](C)NC(=O)[C@@H]2C[C@@H](O)CN2C(=O)[C@H](C(C)C)n2cc(CCCCN3CCC(c4ccc5c(c4)-n4c(nc(=O)c6c(Cl)cccc64)C5(C)C)CC3)nn2)cc1] | (2S,4R)-1-((S)-2-(4-(4-(4-(4-chloro-7,7- dimethyl-5-oxo-5,7-dihydroindolo[1,2- a]quinazolin-10-yl)piperidin-1-yl)butyl)- 1H-1,2,3-triazol-1-yl)-3-methylbutanoyl)- 4-hydroxy-N-((S)-1-(4-(4-methylthiazol- 5-yl)phenyl)ethyl)pyrrolidine-2- carboxamide |
| B74 | 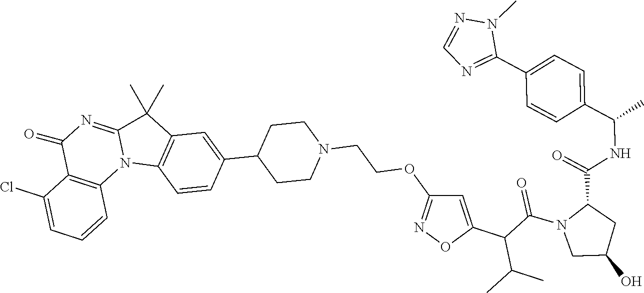 [SMILES: CC(C)C(C(=O)N1C[C@H](O)C[C@H]1C(=O)N[C@@H](C)c1ccc(-c2ncnn2C)cc1)c1cc(OCCN2CCC(c3ccc4c(c3)C(C)(C)c3nc(=O)c5c(Cl)cccc5n3-4)CC2)no1] | (2S,4R)-1-(2-(3-(2-(4-(4-chloro-7,7- dimethyl-5-oxo-5,7-dihydroindolo[1,2- a]quinazolin-9-yl)piperidin-1- yl)ethoxy)isoxazol-5-yl)-3- methylbutanoyl)-4-hydroxy-N-((S)-1-(4- (1-methyl-1H-1,2,4-triazol-5- yl)phenyl)ethyl)pyrrolidine-2- carboxamide |
| B75 | 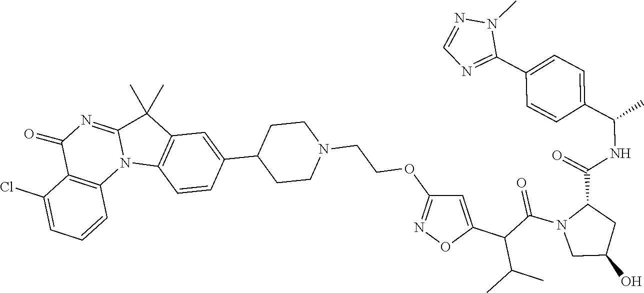 [SMILES: CC(C)C(C(=O)N1C[C@H](O)C[C@H]1C(=O)N[C@@H](C)c1ccc(-c2ncnn2C)cc1)c1cc(OCCN2CCC(c3ccc4c(c3)C(C)(C)c3nc(=O)c5c(Cl)cccc5n3-4)CC2)no1] | (2S,4R)-1-(2-(3-(2-(4-(4-chloro-7,7- dimethyl-5-oxo-5,7-dihydroindolo[1,2- a]quinazolin-9-yl)piperidin-1- yl)ethoxy)isoxazol-5-yl)-3- methylbutanoyl)-4-hydroxy-N-((S)-1-(4- (1-methyl-1H-1,2,4-triazol-5- yl)phenyl)ethyl)pyrrolidine-2- carboxamide |
| B76 | 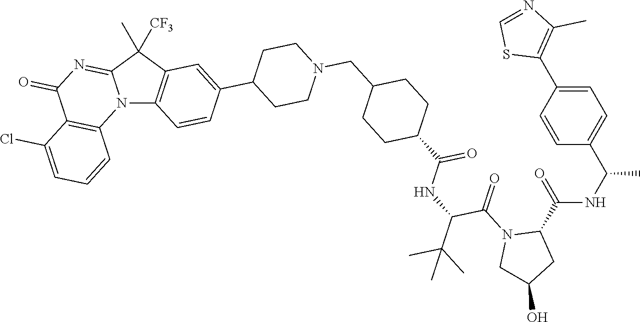 | (2S,4R)-1-((2S)-2-((1r,4S)-4-((4-(4- chloro-7-methyl-5-oxo-7- (trifluoromethyl)-5,7-dihydroindolo[1,2- a]quinazolin-9-yl)piperidin-1- yl)methyl)cyclohexane-1-carboxamido)- 3,3-dimethylbutanoyl)-4-hydroxy-N-((S)- 1-(4-(4-methylthiazol-5- yl)phenyl)ethyl)pyrrolidine-2- carboxamide |
| B77 | 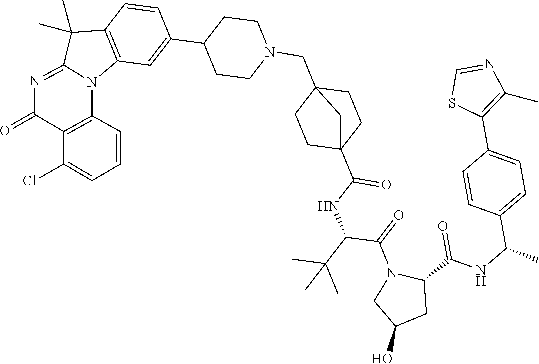 [SMILES: Cc1ncsc1-c1ccc([C@H](C)NC(=O)[C@@H]2C[C@@H](O)CN2C(=O)[C@@H](NC(=O)C23CCC(CN4CCC(c5ccc6c(c5)-n5c(nc(=O)c7c(Cl)cccc75)C6(C)C)CC4)(CC2)C3)C(C)(C)C)cc1] | (2S,4R)-1-((S)-2-(4-((4-(4-chloro-7,7- dimethyl-5-oxo-5,7-dihydroindolo[1,2- a]quinazolin-10-yl)piperidin-1- yl)methyl)bicyclo[2.2.1]heptane-1- carboxamido)-3,3-dimethylbutanoyl)-4- hydroxy-N-((S)-1-(4-(4-methylthiazol-5- yl)phenyl)ethyl)pyrrolidine-2- carboxamide |
| B78 | 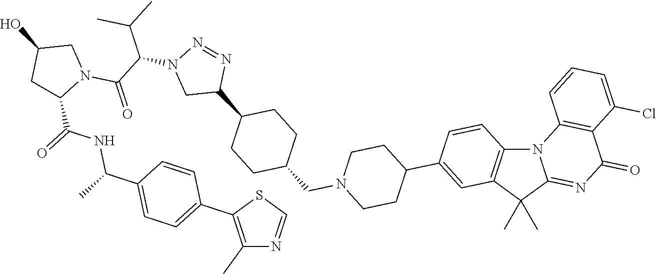 [SMILES: Cc1ncsc1-c1ccc([C@H](C)NC(=O)[C@@H]2C[C@@H](O)CN2C(=O)[C@H](C(C)C)N2CC([C@H]3CC[C@H](CN4CCC(c5ccc6c(c5)C(C)(C)c5nc(=O)c7c(Cl)cccc7n5-6)CC4)CC3)N=N2)cc1] | (2S,4R)-1-((S)-2-(4-((1r,4S)-4-((4-(4- chloro-7,7-dimethyl-5-oxo-5,7- dihydroindolo[1,2-a]quinazolin-9- yl)piperidin-1-yl)methyl)cyclohexyl)-1H- 1,2,3-triazol-1-yl)-3-methylbutanoyl)-4- hydroxy-N-((S)-1-(4-(4-methylthiazol-5- yl)phenyl)ethyl)pyrrolidine-2- carboxamide |
| B79 | 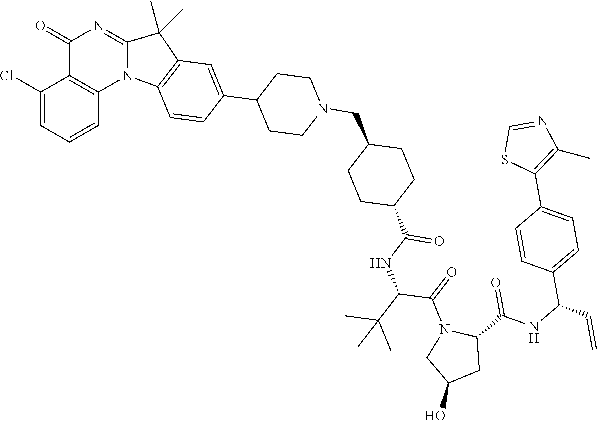 [SMILES: C=C[C@H](NC(=O)[C@@H]1C[C@@H](O)CN1C(=O)[C@@H](NC(=O)[C@H]1CC[C@H](CN2CCC(c3ccc4c(c3)C(C)(C)c3nc(=O)c5c(Cl)cccc5n3-4)CC2)CC1)C(C)(C)C)c1ccc(-c2scnc2C)cc1] | (2S,4R)-1-((S)-2-((11,4S)-4-((4-(4- chloro-7,7-dimethyl-5-oxo-5,7- dihydroindolo[1,2-a]quinazolin-9- yl)piperidin-1-yl)methyl)cyclohexane-1- carboxamido)-3,3-dimethylbutanoyl)-4- hydroxy-N-((S)-1-(4-(4-methylthiazol-5- yl)phenyl)allyl)pyrrolidine-2- carboxamide |
| B80 | 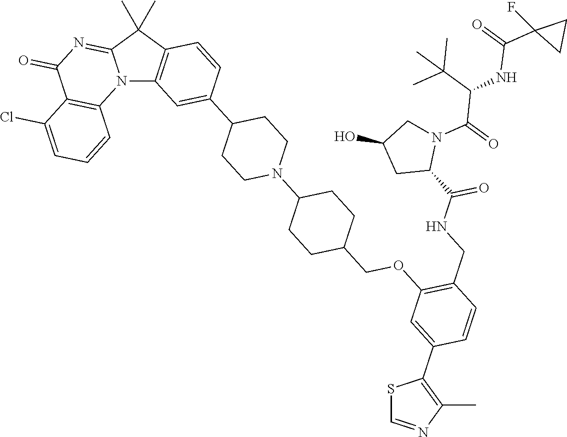 [SMILES: Cc1ncsc1-c1ccc(CNC(=O)[C@@H]2C[C@@H](O)CN2C(=O)[C@@H](NC(=O)C2(F)CC2)C(C)(C)C)c(OCC2CCC(N3CCC(c4ccc5c(c4)-n4c(nc(=O)c6c(Cl)cccc64)C5(C)C)CC3)CC2)c1] | (2S,4R)-N-(2-((4-(4-(4-chloro-7,7- dimethyl-5-oxo-5,7-dihydroindolo[1,2- a]quinazolin-10-yl)piperidin-1- yl)cyclohexyl)methoxy)-4-(4- methylthiazol-5-yl)benzyl)-1-((S)-2-(1- fluorocyclopropane-1-carboxamido)-3,3- dimethylbutanoyl)-4-hydroxypyrrolidine- 2-carboxamide |
| B81 | 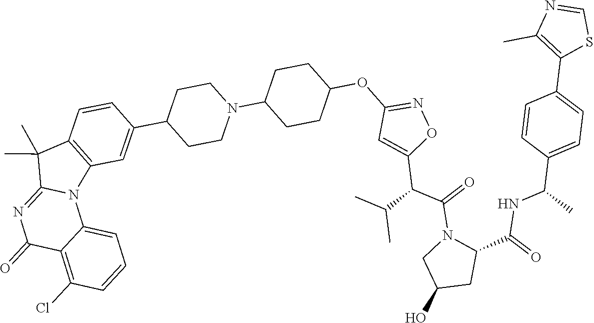 [SMILES: Cc1ncsc1-c1ccc([C@H](C)NC(=O)[C@@H]2C[C@@H](O)CN2C(=O)[C@@H](c2cc(OC3CCC(N4CCC(c5ccc6c(c5)-n5c(nc(=O)c7c(Cl)cccc75)C6(C)C)CC4)CC3)no2)C(C)C)cc1] | (2S,4R)-1-((R)-2-(3-((4-(4-(4-chloro-7,7- dimethyl-5-oxo-5,7-dihydroindolo[1,2- a]quinazolin-10-yl)piperidin-1- yl)cyclohexyl)oxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B82 | 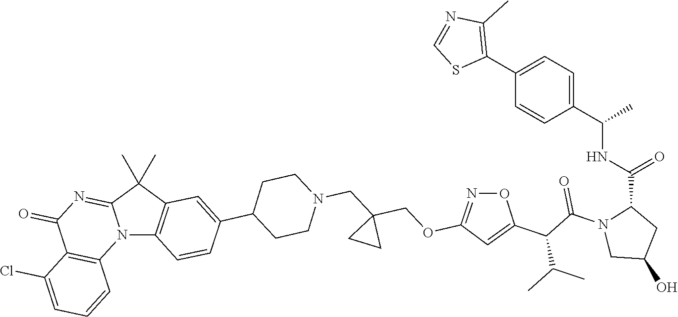 [SMILES: Cc1ncsc1-c1ccc([C@H](C)NC(=O)[C@@H]2C[C@@H](O)CN2C(=O)[C@@H](c2cc(OCC3(CN4CCC(c5ccc6c(c5)C(C)(C)c5nc(=O)c7c(Cl)cccc7n5-6)CC4)CC3)no2)C(C)C)cc1] | (2S,4R)-1-((R)-2-(3-((1-((4-(4-chloro- 7,7-dimethyl-5-oxo-5,7- dihydroindolo[1,2-a]quinazolin-9- yl)piperidin-1- yl)methyl)cyclopropyl)methoxy)isoxazol- 5-yl)-3-methylbutanoyl)-4-hydroxy-N- ((S)-1-(4-(4-methylthiazol-5- yl)phenyl)ethyl)pyrrolidine-2- carboxamide |
| B83 | 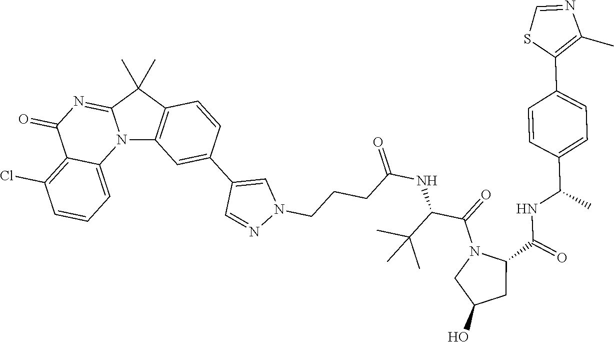 [SMILES: Cc1ncsc1-c1ccc([C@H](C)NC(=O)[C@@H]2C[C@@H](O)CN2C(=O)[C@@H](NC(=O)CCCn2cc(-c3ccc4c(c3)-n3c(nc(=O)c5c(Cl)cccc53)C4(C)C)cn2)C(C)(C)C)cc1] | (2S,4R)-1-((S)-2-(4-(4-(4-chloro-7,7- dimethyl-5-oxo-5,7-dihydroindolo[1,2- a]quinazolin-10-yl)-1H-pyrazol-1- yl)butanamido)-3,3-dimethylbutanoyl)-4- hydroxy-N-((S)-1-(4-(4-methylthiazol-5- yl)phenyl)ethyl)pyrrolidine-2- carboxamide |
| B84 | 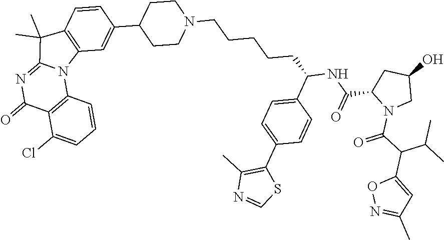 [SMILES: Cc1cc(C(C(=O)N2C[C@H](O)C[C@H]2C(=O)N[C@@H](CCCCCN2CCC(c3ccc4c(c3)-n3c(nc(=O)c5c(Cl)cccc53)C4(C)C)CC2)c2ccc(-c3scnc3C)cc2)C(C)C)on1] | (2S,4R)-N-((S)-6-(4-(4-chloro-7,7- dimethyl-5-oxo-5,7-dihydroindolo[1,2- a]quinazolin-10-yl)piperidin-1-yl)-1-(4- (4-methylthiazol-5-yl)phenyl)hexyl)-4- hydroxy-1-(3-methyl-2-(3- methylisoxazol-5- yl)butanoyl)pyrrolidine-2-carboxamide |
| B85 | 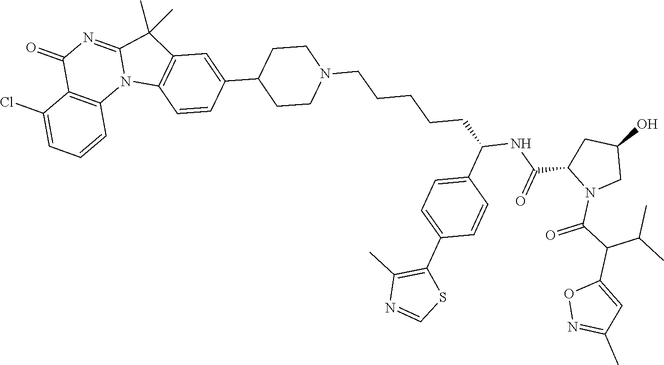 [SMILES: Cc1cc(C(C(=O)N2C[C@H](O)C[C@H]2C(=O)N[C@@H](CCCCCN2CCC(c3ccc4c(c3)C(C)(C)c3nc(=O)c5c(Cl)cccc5n3-4)CC2)c2ccc(-c3scnc3C)cc2)C(C)C)on1] | (2S,4R)-N-((S)-6-(4-(4-chloro-7,7- dimethyl-5-oxo-5,7-dihydroindolo[1,2- a]quinazolin-9-yl)piperidin-1-yl)-1-(4-(4- methylthiazol-5-yl)phenyl)hexyl)-4- hydroxy-1-(3-methyl-2-(3- methylisoxazol-5- yl)butanoyl)pyrrolidine-2-carboxamide |
| B86 | 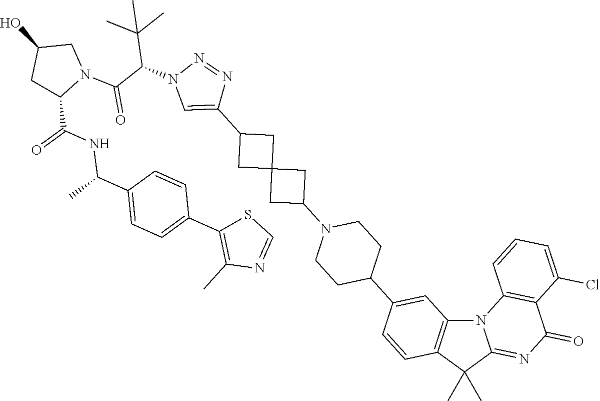 [SMILES: Cc1ncsc1-c1ccc([C@H](C)NC(=O)[C@@H]2C[C@@H](O)CN2C(=O)[C@@H](n2cc(C3CC4(C3)CC(N3CCC(c5ccc6c(c5)-n5c(nc(=O)c7c(Cl)cccc75)C6(C)C)CC3)C4)nn2)C(C)(C)C)cc1] | (2S,4R)-1-((S)-2-(4-(6-(4-(4-chloro-7,7- dimethyl-5-oxo-5,7-dihydroindolo[1,2- a]quinazolin-10-yl)piperidin-1- yl)spiro[3.3]heptan-2-yl)-1H-1,2,3- triazol-1-yl)-3,3-dimethylbutanoyl)-4- hydroxy-N-((S)-1-(4-(4-methylthiazol-5- yl)phenyl)ethyl)pyrrolidine-2- carboxamide |
| B87 | 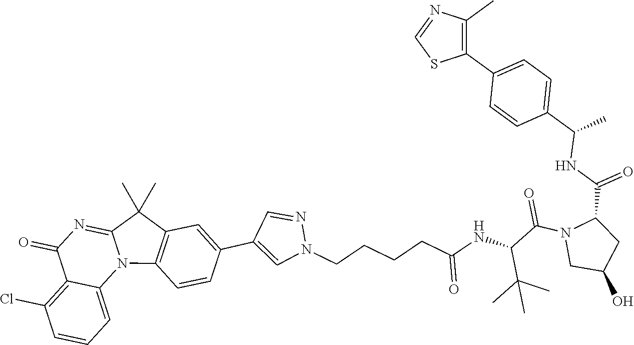 [SMILES: Cc1ncsc1-c1ccc([C@H](C)NC(=O)[C@@H]2C[C@@H](O)CN2C(=O)[C@@H](NC(=O)CCCCn2cc(-c3ccc4c(c3)C(C)(C)c3nc(=O)c5c(Cl)cccc5n3-4)cn2)C(C)(C)C)cc1] | (2S,4R)-1-((S)-2-(5-(4-(4-chloro-7,7- dimethyl-5-oxo-5,7-dihydroindolo[1,2- a]quinazolin-9-yl)-1H-pyrazol-1- yl)pentanamido)-3,3-dimethylbutanoyl)- 4-hydroxy-N-((S)-1-(4-(4-methylthiazol- 5-yl)phenyl)ethyl)pyrrolidine-2- carboxamide |
| B88 | 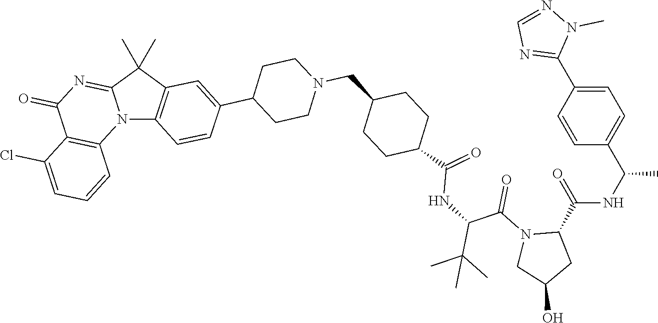 [SMILES: C[C@H](NC(=O)[C@@H]1C[C@@H](O)CN1C(=O)[C@@H](NC(=O)[C@H]1CC[C@H](CN2CCC(c3ccc4c(c3)C(C)(C)c3nc(=O)c5c(Cl)cccc5n3-4)CC2)CC1)C(C)(C)C)c1ccc(-c2ncnn2C)cc1] | (2S,4R)-1-((S)-2-((11,4S)-4-((4-(4- chloro-7,7-dimethyl-5-oxo-5,7- dihydroindolo[1,2-a]quinazolin-9- yl)piperidin-1-yl)methyl)cyclohexane-1- carboxamido)-3,3-dimethylbutanoyl)-4- hydroxy-N-((S)-1-(4-(1-methyl-1H-1,2,4- triazol-5-yl)phenyl)ethyl)pyrrolidine-2- carboxamide |
| B89 | 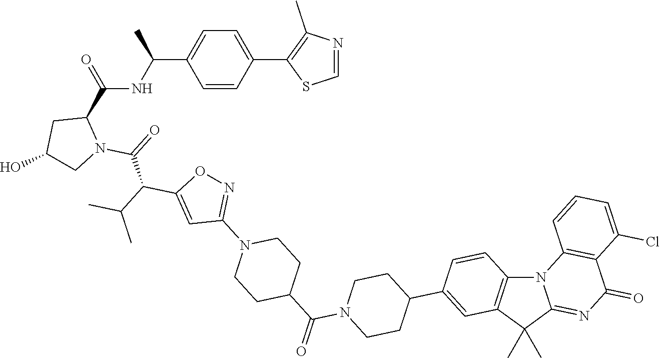 [SMILES: Cc1ncsc1-c1ccc([C@H](C)NC(=O)[C@@H]2C[C@@H](O)CN2C(=O)[C@H](c2cc(N3CCC(C(=O)N4CCC(c5ccc6c(c5)C(C)(C)c5nc(=O)c7c(Cl)cccc7n5-6)CC4)CC3)no2)C(C)C)cc1] | (2S,4R)-1-((S)-2-(3-(4-(4-(4-chloro-7,7- dimethyl-5-oxo-5,7-dihydroindolo[1,2- a]quinazolin-9-yl)piperidine-1- carbonyl)piperidin-1-yl)isoxazol-5-yl)-3- methylbutanoyl)-4-hydroxy-N-((S)-1-(4- (4-methylthiazol-5- yl)phenyl)ethyl)pyrrolidine-2- carboxamide |
| B90 | 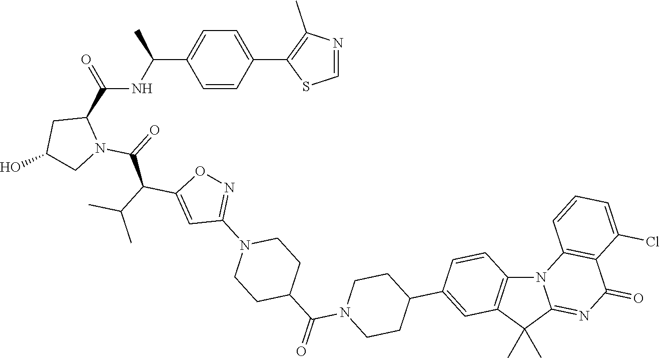 [SMILES: Cc1ncsc1-c1ccc([C@H](C)NC(=O)[C@@H]2C[C@@H](O)CN2C(=O)[C@@H](c2cc(N3CCC(C(=O)N4CCC(c5ccc6c(c5)C(C)(C)c5nc(=O)c7c(Cl)cccc7n5-6)CC4)CC3)no2)C(C)C)cc1] | (2S,4R)-1-((R)-2-(3-(4-(4-(4-chloro-7,7- dimethyl-5-oxo-5,7-dihydroindolo[1,2- a]quinazolin-9-yl)piperidine-1- carbonyl)piperidin-1-yl)isoxazol-5-yl)-3- methylbutanoyl)-4-hydroxy-N-((S)-1-(4- (4-methylthiazol-5- yl)phenyl)ethyl)pyrrolidine-2- carboxamide |
| B91 | 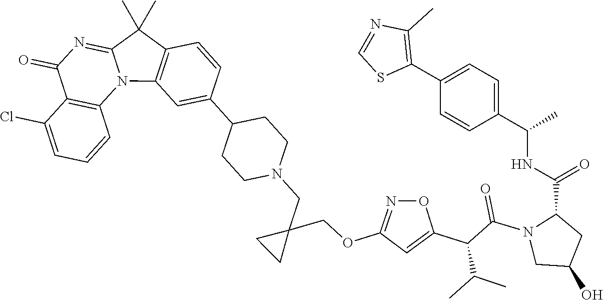 [SMILES: Cc1ncsc1-c1ccc([C@H](C)NC(=O)[C@@H]2C[C@@H](O)CN2C(=O)[C@@H](c2cc(OCC3(CN4CCC(c5ccc6c(c5)-n5c(nc(=O)c7c(Cl)cccc75)C6(C)C)CC4)CC3)no2)C(C)C)cc1] | (2S,4R)-1-((R)-2-(3-((1-((4-(4-chloro- 7,7-dimethyl-5-oxo-5,7- dihydroindolo[1,2-a]quinazolin-10- yl)piperidin-1- yl)methyl)cyclopropyl)methoxy)isoxazol- 5-yl)-3-methylbutanoyl)-4-hydroxy-N- ((S)-1-(4-(4-methylthiazol-5- yl)phenyl)ethyl)pyrrolidine-2- carboxamide |
| B92 | 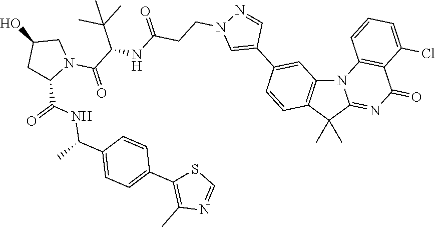 [SMILES: Cc1ncsc1-c1ccc([C@H](C)NC(=O)[C@@H]2C[C@@H](O)CN2C(=O)[C@@H](NC(=O)CCn2cc(-c3ccc4c(c3)-n3c(nc(=O)c5c(Cl)cccc53)C4(C)C)cn2)C(C)(C)C)cc1] | (2S,4R)-1-((S)-2-(3-(4-(4-chloro-7,7- dimethyl-5-oxo-5,7-dihydroindolo[1,2- a]quinazolin-10-yl)-1H-pyrazol-1- yl)propanamido)-3,3-dimethylbutanoyl)- 4-hydroxy-N-((S)-1-(4-(4-methylthiazol- 5-yl)phenyl)ethyl)pyrrolidine-2- carboxamide |
| B93 | 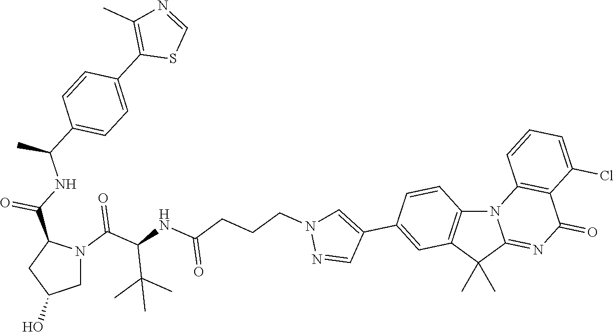 [SMILES: Cc1ncsc1-c1ccc([C@H](C)NC(=O)[C@@H]2C[C@@H](O)CN2C(=O)[C@@H](NC(=O)CCCn2cc(-c3ccc4c(c3)C(C)(C)c3nc(=O)c5c(Cl)cccc5n3-4)cn2)C(C)(C)C)cc1] | (2S,4R)-1-((S)-2-(4-(4-(4-chloro-7,7- dimethyl-5-oxo-5,7-dihydroindolo[1,2- a]quinazolin-9-yl)-1H-pyrazol-1- yl)butanamido)-3,3-dimethylbutanoyl)-4- hydroxy-N-((S)-1-(4-(4-methylthiazol-5- yl)phenyl)ethyl)pyrrolidine-2- carboxamide |
| B94 | 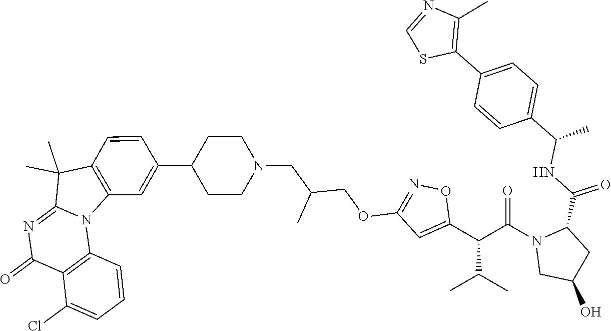 [SMILES: Cc1ncsc1-c1ccc([C@H](C)NC(=O)[C@@H]2C[C@@H](O)CN2C(=O)[C@@H](c2cc(OCC(C)CN3CCC(c4ccc5c(c4)-n4c(nc(=O)c6c(Cl)cccc64)C5(C)C)CC3)no2)C(C)C)cc1] | (2S,4R)-1-((2R)-2-(3-(3-(4-(4-chloro-7,7- dimethyl-5-oxo-5,7-dihydroindolo[1,2- a]quinazolin-9-yl)piperidin-1-yl)-2- methylpropoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B95 | 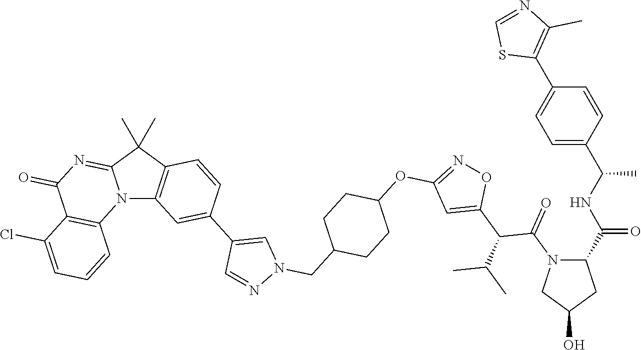 [SMILES: Cc1ncsc1-c1ccc([C@H](C)NC(=O)[C@@H]2C[C@@H](O)CN2C(=O)[C@@H](c2cc(OC3CCC(Cn4cc(-c5ccc6c(c5)-n5c(nc(=O)c7c(Cl)cccc75)C6(C)C)cn4)CC3)no2)C(C)C)cc1] | (2S,4R)-1-((R)-2-(3-((4-((4-(4-chloro- 7,7-dimethyl-5-oxo-5,7- dihydroindolo[1,2-a]quinazolin-10-yl)- 1H-pyrazol-1- yl)methyl)cyclohexyl)oxy)isoxazol-5-yl)- 3-methylbutanoyl)-4-hydroxy-N-((S)-1- (4-(4-methylthiazol-5- yl)phenyl)ethyl)pyrrolidine-2- carboxamide |
| B96 | 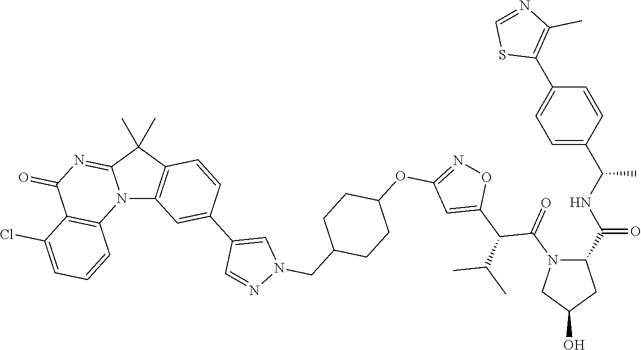 [SMILES: Cc1ncsc1-c1ccc([C@H](C)NC(=O)[C@@H]2C[C@@H](O)CN2C(=O)[C@@H](c2cc(OC3CCC(Cn4cc(-c5ccc6c(c5)-n5c(nc(=O)c7c(Cl)cccc75)C6(C)C)cn4)CC3)no2)C(C)C)cc1] | (2S,4R)-1-((R)-2-(3-((4-((4-(4-chloro- 7,7-dimethyl-5-oxo-5,7- dihydroindolo[1,2-a]quinazolin-10-yl)- 1H-pyrazol-1- yl)methyl)cyclohexyl)oxy)isoxazol-5-yl)- 3-methylbutanoyl)-4-hydroxy-N-((S)-1- (4-(4-methylthiazol-5- yl)phenyl)ethyl)pyrrolidine-2- carboxamide |
| B97 | 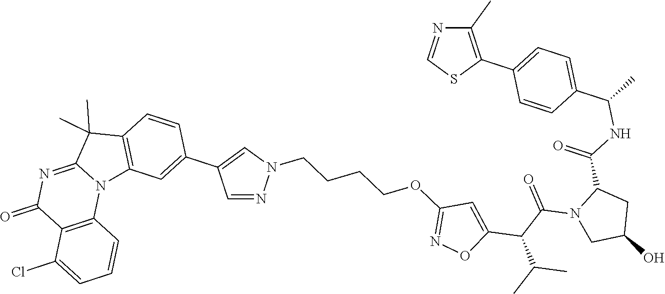 | (2S,4R)-1-((R)-2-(3-(4-(4-(4-chloro-7,7- dimethyl-5-oxo-5,7-dihydroindolo[1,2- a]quinazolin-10-yl)-1H-pyrazol-1- yl)butoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B98 | 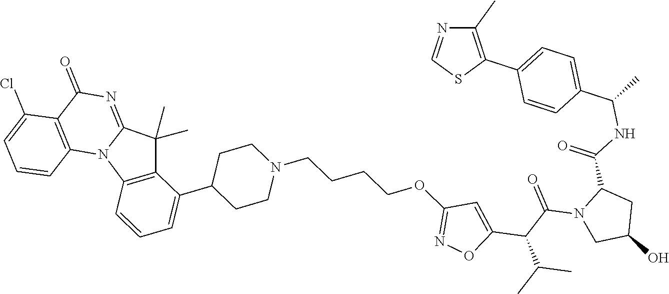 | (2S,4R)-1-(2-(3-(4-(4-(4-chloro-7,7- dimethyl-5-oxo-5,7-dihydroindolo[1,2- a]quinazolin-8-yl)piperidin-1- yl)butoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B99 | 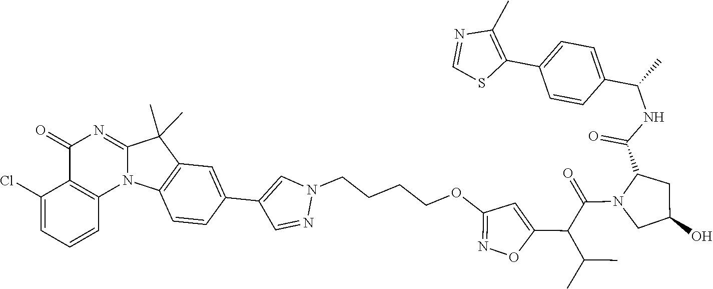 | (2S,4R)-1-(2-(3-(4-(4-(4-chloro-7,7- dimethyl-5-oxo-5,7-dihydroindolo[1,2- a]quinazolin-9-yl)-1H-pyrazol-1- yl)butoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B100 | 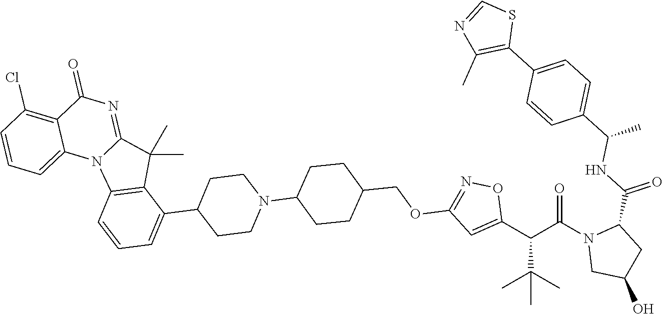 [SMILES: Cc1ncsc1-c1ccc([C@H](C)NC(=O)[C@@H]2C[C@@H](O)CN2C(=O)[C@@H](c2cc(OCC3CCC(N4CCC(c5cccc6c5C(C)(C)c5nc(=O)c7c(Cl)cccc7n5-6)CC4)CC3)no2)C(C)(C)C)cc1] | (2S,4R)-1-((R)-2-(3-((4-(4-(4-chloro-7,7- dimethyl-5-oxo-5,7-dihydroindolo[1,2- a]quinazolin-8-yl)piperidin-1- yl)cyclohexyl)methoxy)isoxazol-5-yl)-3- methylbutanoyl)-4-hydroxy-N-((S)-1-(4- (4-methylthiazol-5- yl)phenyl)ethyl)pyrrolidine-2- carboxamide |
| B101 | 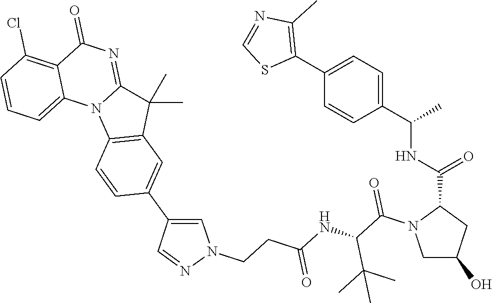 [SMILES: Cc1ncsc1-c1ccc([C@H](C)NC(=O)[C@@H]2C[C@@H](O)CN2C(=O)[C@@H](NC(=O)CCn2cc(-c3ccc4c(c3)C(C)(C)c3nc(=O)c5c(Cl)cccc5n3-4)cn2)C(C)(C)C)cc1] | (2S,4R)-1-((S)-2-(3-(4-(4-chloro-7,7- dimethyl-5-oxo-5,7-dihydroindolo[1,2- a]quinazolin-9-yl)-1H-pyrazol-1- yl)propanamido)-3,3-dimethylbutanoyl)- 4-hydroxy-N-((S)-1-(4-(4-methylthiazol- 5-yl)phenyl)ethyl)pyrrolidine-2- carboxamide |
| B102 | 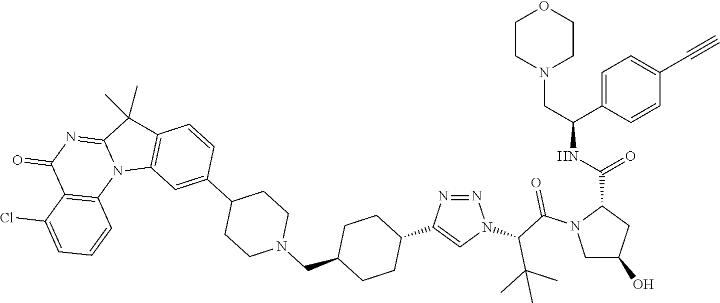 [SMILES: C#Cc1ccc([C@H](CN2CCOCC2)NC(=O)[C@@H]2C[C@@H](O)CN2C(=O)[C@@H](n2cc([C@H]3CC[C@H](CN4CCC(c5ccc6c(c5)-n5c(nc(=O)c7c(Cl)cccc75)C6(C)C)CC4)CC3)nn2)C(C)(C)C)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- N-((R)-1-(4-ethynylphenyl)-2- morpholinoethyl)-4-hydroxypyrrolidine- 2-carboxamide |
| B103 | 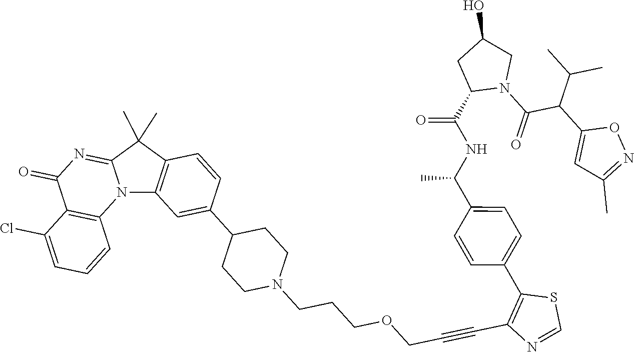 [SMILES: Cc1cc(C(C(=O)N2C[C@H](O)C[C@H]2C(=O)N[C@@H](C)c2ccc(-c3scnc3C#CCOCCCN3CCC(c4ccc5c(c4)-n4c(nc(=O)c6c(Cl)cccc64)C5(C)C)CC3)cc2)C(C)C)on1] | (2S,4R)-N-((S)-1-(4-(4-(3-(3-(4-(4- chloro-7,7-dimethyl-5-oxo-5,7- dihydroindolo[1,2-a]quinazolin-10- yl)piperidin-1-yl)propoxy)prop-1-yn-1- yl)thiazol-5-yl)phenyl)ethyl)-4-hydroxy- 1-(3-methyl-2-(3-methylisoxazol-5- yl)butanoyl)pyrrolidine-2-carboxamide |
| B104 | 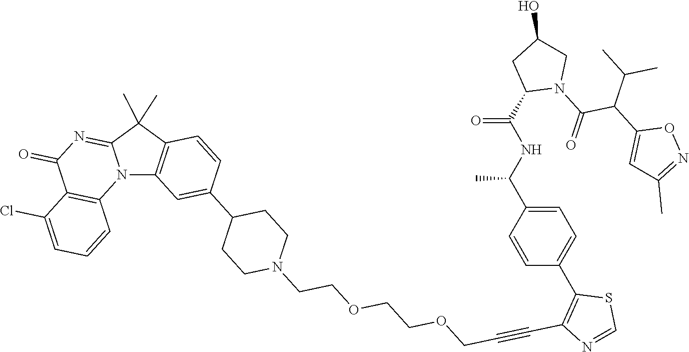 [SMILES: Cc1cc(C(C(=O)N2C[C@H](O)C[C@H]2C(=O)N[C@@H](C)c2ccc(-c3scnc3C#CCOCCOCCN3CCC(c4ccc5c(c4)-n4c(nc(=O)c6c(Cl)cccc64)C5(C)C)CC3)cc2)C(C)C)on1] | (2S,4R)-N-((S)-1-(4-(4-(3-(2-(2-(4-(4- chloro-7,7-dimethyl-5-oxo-5,7- dihydroindolo[1,2-a]quinazolin-10- yl)piperidin-1-yl)ethoxy)ethoxy)prop-1- yn-1-yl)thiazol-5-yl)phenyl)ethyl)-4- hydroxy-1-(3-methyl-2-(3- methylisoxazol-5- yl)butanoyl)pyrrolidine-2-carboxamide |
| B105 | 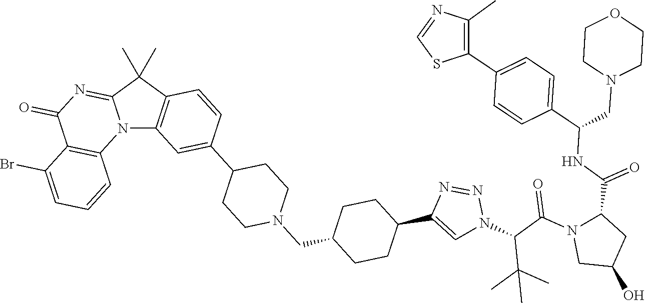 [SMILES: Cc1ncsc1-c1ccc([C@H](CN2CCOCC2)NC(=O)[C@@H]2C[C@@H](O)CN2C(=O)[C@@H](n2cc([C@H]3CC[C@H](CN4CCC(c5ccc6c(c5)-n5c(nc(=O)c7c(Br)cccc75)C6(C)C)CC4)CC3)nn2)C(C)(C)C)cc1] | (2S,4R)-1-((S)-2-(4-((1r,4S)-4-((4-(4- bromo-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- 4-hydroxy-N-((R)-1-(4-(4-methylthiazol- 5-yl)phenyl)-2- morpholinoethyl)pyrrolidine-2- carboxamide |
| B106 | 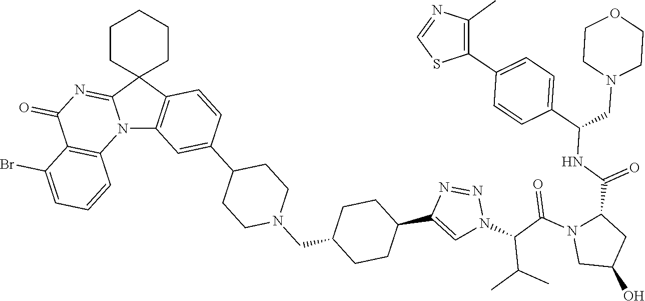 [SMILES: Cc1ncsc1-c1ccc([C@H](CN2CCOCC2)NC(=O)[C@@H]2C[C@@H](O)CN2C(=O)[C@H](C(C)C)n2cc([C@H]3CC[C@H](CN4CCC(c5ccc6c(c5)-n5c(nc(=O)c7c(Br)cccc75)C65CCCCC5)CC4)CC3)nn2)cc1] | (2S,4R)-1-((S)-2-(4-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexyl)-1H- 1,2,3-triazol-1-yl)-3-methylbutanoyl)-4- hydroxy-N-((R)-1-(4-(4-methylthiazol-5- yl)phenyl)-2- morpholinoethyl)pyrrolidine-2- carboxamide |
| B107 | 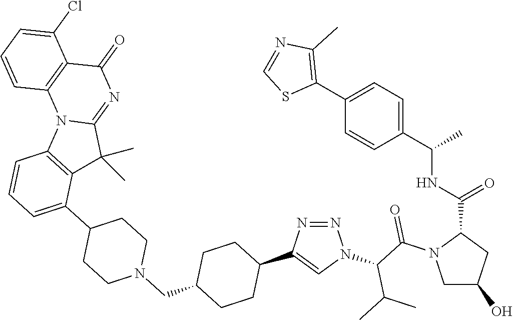 [SMILES: Cc1ncsc1-c1ccc([C@H](C)NC(=O)[C@@H]2C[C@@H](O)CN2C(=O)[C@H](C(C)C)n2cc([C@H]3CC[C@H](CN4CCC(c5cccc6c5C(C)(C)c5nc(=O)c7c(Cl)cccc7n5-6)CC4)CC3)nn2)cc1] | (2S,4R)-1-((S)-2-(4-((1r,4S)-4-((4-(4- chloro-7,7-dimethyl-5-oxo-5,7- dihydroindolo[1,2-a]quinazolin-8- yl)piperidin-1-yl)methyl)cyclohexyl)-1H- 1,2,3-triazol-1-yl)-3-methylbutanoyl)-4- hydroxy-N-((S)-1-(4-(4-methylthiazol-5- yl)phenyl)ethyl)pyrrolidine-2- carboxamide |
| B108 | 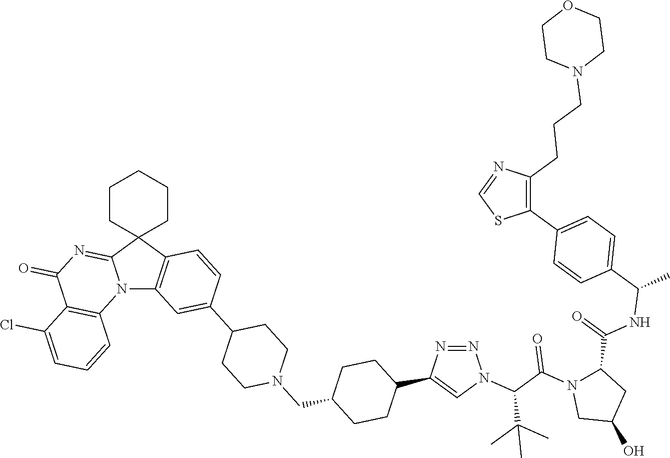 | (2S,4R)-1-((S)-2-(4-((11,4S)-4-((4-(4′- chloro-5′-oxo-5′H-spiro[cyclohexane- 1,7′-indolo[1,2-a]quinazolin]-10′- yl)piperidin-1-yl)methyl)cyclohexyl)-1H- 1,2,3-triazol-1-yl)-3,3-dimethylbutanoyl)- 4-hydroxy-N-((S)-1-(4-(4-(3- morpholinopropyl)thiazol-5- yl)phenyl)ethyl)pyrrolidine-2- carboxamide |
| B109 | 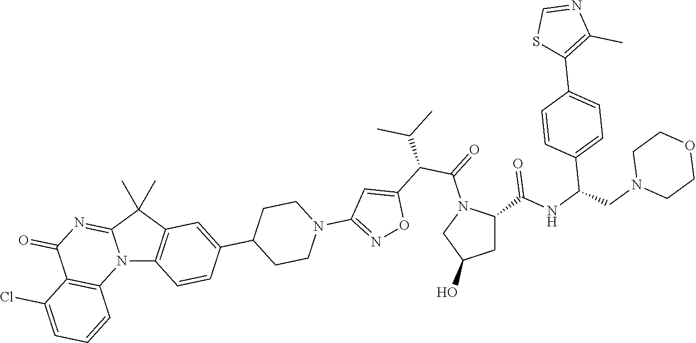 [SMILES: Cc1ncsc1-c1ccc([C@H](CN2CCOCC2)NC(=O)[C@@H]2C[C@@H](O)CN2C(=O)[C@H](c2cc(N3CCC(c4ccc5c(c4)C(C)(C)c4nc(=O)c6c(Cl)cccc6n4-5)CC3)no2)C(C)C)cc1] | (2S,4R)-1-((S)-2-(3-(4-(4-chloro-7,7- dimethyl-5-oxo-5,7-dihydroindolo[1,2- a]quinazolin-9-yl)piperidin-1-yl)isoxazol- 5-yl)-3-methylbutanoyl)-4-hydroxy-N- ((R)-1-(4-(4-methylthiazol-5-yl)phenyl)- 2-morpholinoethyl)pyrrolidine-2- carboxamide |
| B110 | 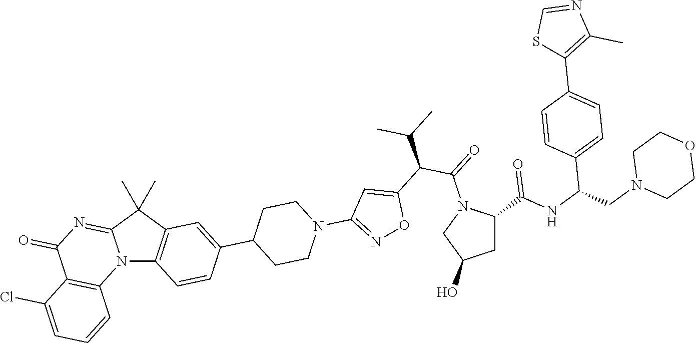 [SMILES: Cc1ncsc1-c1ccc([C@H](CN2CCOCC2)NC(=O)[C@@H]2C[C@@H](O)CN2C(=O)[C@@H](c2cc(N3CCC(c4ccc5c(c4)C(C)(C)c4nc(=O)c6c(Cl)cccc6n4-5)CC3)no2)C(C)C)cc1] | (2S,4R)-1-((R)-2-(3-(4-(4-chloro-7,7- dimethyl-5-oxo-5,7-dihydroindolo[1,2- a]quinazolin-9-yl)piperidin-1-yl)isoxazol- 5-yl)-3-methylbutanoyl)-4-hydroxy-N- ((R)-1-(4-(4-methylthiazol-5-yl)phenyl)- 2-morpholinoethyl)pyrrolidine-2- carboxamide |
| B111 | 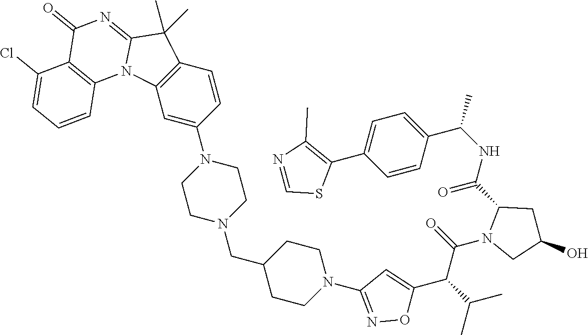 [SMILES: Cc1ncsc1-c1ccc([C@H](C)NC(=O)[C@@H]2C[C@@H](O)CN2C(=O)[C@@H](c2cc(N3CCC(CN4CCN(c5ccc6c(c5)-n5c(nc(=O)c7c(Cl)cccc75)C6(C)C)CC4)CC3)no2)C(C)C)cc1] | (2S,4R)-1-((R)-2-(3-(4-((4-(4-chloro-7,7- dimethyl-5-oxo-5,7-dihydroindolo[1,2- a]quinazolin-10-yl)piperazin-1- yl)methyl)piperidin-1-yl)isoxazol-5-yl)- 3-methylbutanoyl)-4-hydroxy-N-((S)-1- (4-(4-methylthiazol-5- yl)phenyl)ethyl)pyrrolidine-2- carboxamide |
| B112 | 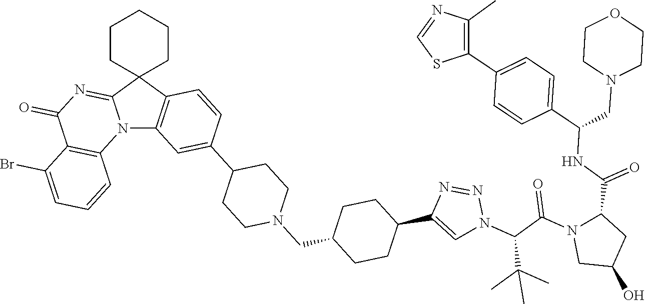 [SMILES: Cc1ncsc1-c1ccc([C@H](CN2CCOCC2)NC(=O)[C@@H]2C[C@@H](O)CN2C(=O)[C@@H](n2cc([C@H]3CC[C@H](CN4CCC(c5ccc6c(c5)-n5c(nc(=O)c7c(Br)cccc75)C65CCCCC5)CC4)CC3)nn2)C(C)(C)C)cc1] | (2S,4R)-1-((S)-2-(4-((1r,4S)-4-((4-(4′- bromo-5′-oxo-5′H-spiro[cyclohexane- 1,7′-indolo[1,2-a]quinazolin]-10′- yl)piperidin-1-yl)methyl)cyclohexyl)-1H- 1,2,3-triazol-1-yl)-3,3-dimethylbutanoyl)- 4-hydroxy-N-((R)-1-(4-(4-methylthiazol- 5-yl)phenyl)-2- morpholinoethyl)pyrrolidine-2- carboxamide |
| B113 | 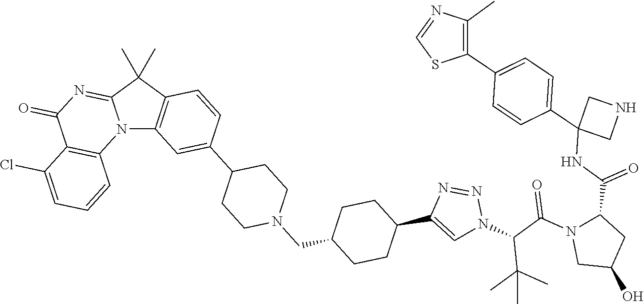 [SMILES: Cc1ncsc1-c1ccc(C2(NC(=O)[C@@H]3C[C@@H](O)CN3C(=O)[C@@H](n3cc([C@H]4CC[C@H](CN5CCC(c6ccc7c(c6)-n6c(nc(=O)c8c(Cl)cccc86)C7(C)C)CC5)CC4)nn3)C(C)(C)C)CNC2)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- 4-hydroxy-N-(3-(4-(4-methylthiazol-5- yl)phenyl)azetidin-3-yl)pyrrolidine-2- carboxamide |
| B114 | 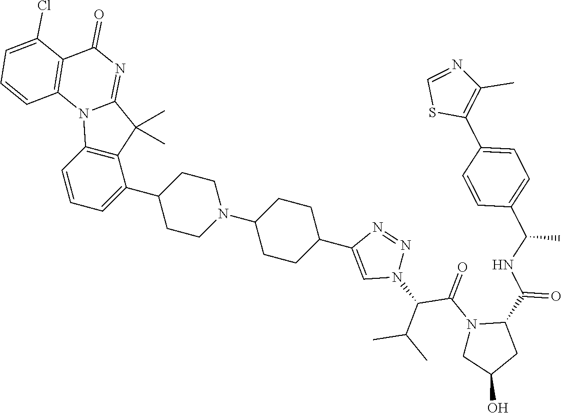 [SMILES: Cc1ncsc1-c1ccc([C@H](C)NC(=O)[C@@H]2C[C@@H](O)CN2C(=O)[C@H](C(C)C)n2cc(C3CCC(N4CCC(c5cccc6c5C(C)(C)c5nc(=O)c7c(Cl)cccc7n5-6)CC4)CC3)nn2)cc1] | (2S,4R)-1-((S)-2-(4-(4-(4-(4-chloro-7,7- dimethyl-5-oxo-5,7-dihydroindolo[1,2- a]quinazolin-8-yl)piperidin-1- yl)cyclohexyl)-1H-1,2,3-triazol-1-yl)-3- methylbutanoyl)-4-hydroxy-N-((S)-1-(4- (4-methylthiazol-5- yl)phenyl)ethyl)pyrrolidine-2- carboxamide |
| B115 | 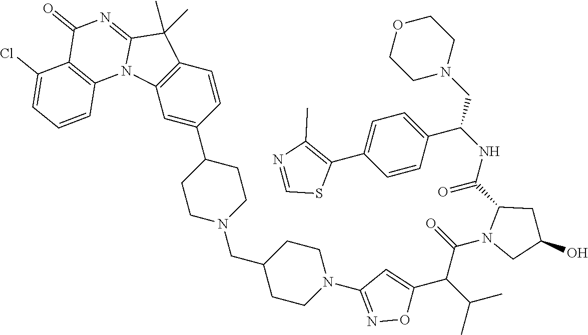 [SMILES: Cc1ncsc1-c1ccc([C@H](CN2CCOCC2)NC(=O)[C@@H]2C[C@@H](O)CN2C(=O)C(c2cc(N3CCC(CN4CCC(c5ccc6c(c5)-n5c(nc(=O)c7c(Cl)cccc75)C6(C)C)CC4)CC3)no2)C(C)C)cc1] | (2S,4R)-1-(2-(3-(4-((4-(4-chloro-7,7- dimethyl-5-oxo-5,7-dihydroindolo[1,2- a]quinazolin-10-yl)piperidin-1- yl)methyl)piperidin-1-yl)isoxazol-5-yl)- 3-methylbutanoyl)-4-hydroxy-N-((R)-1- (4-(4-methylthiazol-5-yl)phenyl)-2- morpholinoethyl)pyrrolidine-2- carboxamide |
| B116 | 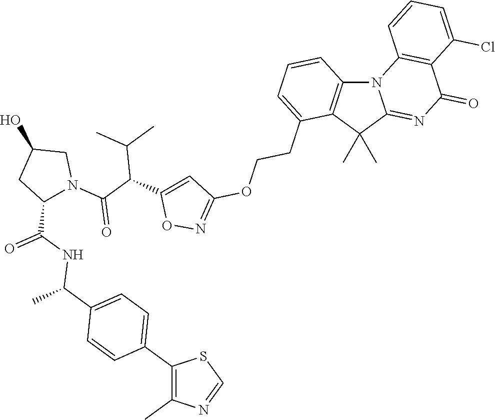 [SMILES: Cc1ncsc1-c1ccc([C@H](C)NC(=O)[C@@H]2C[C@@H](O)CN2C(=O)[C@@H](c2cc(OCCc3cccc4c3C(C)(C)c3nc(=O)c5c(Cl)cccc5n3-4)no2)C(C)C)cc1] | (2S,4R)-1-((R)-2-(3-(2-(4-chloro-7,7- dimethyl-5-oxo-5,7-dihydroindolo[1,2- a]quinazolin-8-yl)ethoxy)isoxazol-5-yl)- 3-methylbutanoyl)-4-hydroxy-N-((S)-1- (4-(4-methylthiazol-5- yl)phenyl)ethyl)pyrrolidine-2- carboxamide |
| B117 | 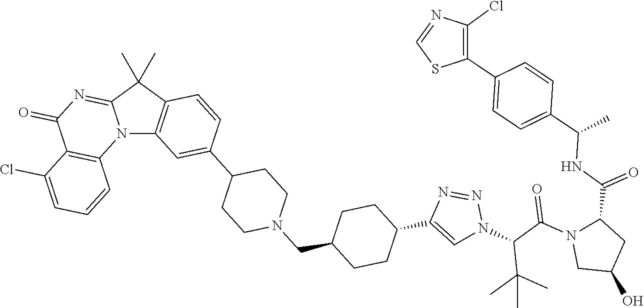 [SMILES: C[C@H](NC(=O)[C@@H]1C[C@@H](O)CN1C(=O)[C@@H](n1cc([C@H]2CC[C@H](CN3CCC(c4ccc5c(c4)-n4c(nc(=O)c6c(Cl)cccc64)C5(C)C)CC3)CC2)nn1)C(C)(C)C)c1ccc(-c2scnc2Cl)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- N-((S)-1-(4-(4-chlorothiazol-5- yl)phenyl)ethyl)-4-hydroxypyrrolidine-2- carboxamide |
| B118 | 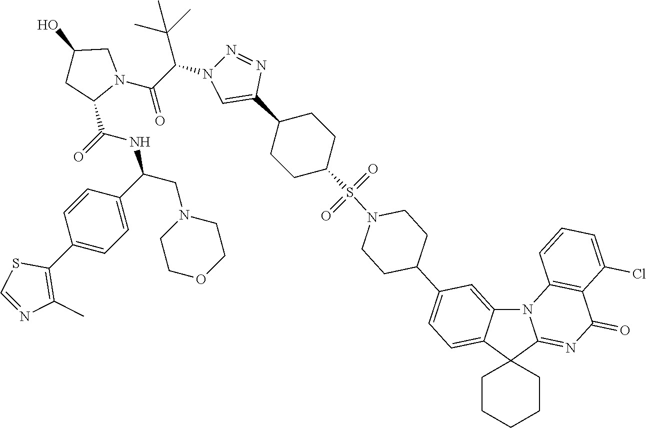 [SMILES: Cc1ncsc1-c1ccc([C@H](CN2CCOCC2)NC(=O)[C@@H]2C[C@@H](O)CN2C(=O)[C@@H](n2cc([C@H]3CC[C@H](S(=O)(=O)N4CCC(c5ccc6c(c5)-n5c(nc(=O)c7c(Cl)cccc75)C65CCCCC5)CC4)CC3)nn2)C(C)(C)C)cc1] | (2S,4R)-1-((S)-2-(4-((1r,4S)-4-((4-(4′- chloro-5′-oxo-5′H-spiro[cyclohexane- 1,7′-indolo[1,2-a]quinazolin]-10′- yl)piperidin-1-yl)sulfonyl)cyclohexyl)- 1H-1,2,3-triazol-1-yl)-3,3- dimethylbutanoyl)-4-hydroxy-N-((R)-1- (4-(4-methylthiazol-5-yl)phenyl)-2- morpholinoethyl)pyrrolidine-2- carboxamide |
| B119 | 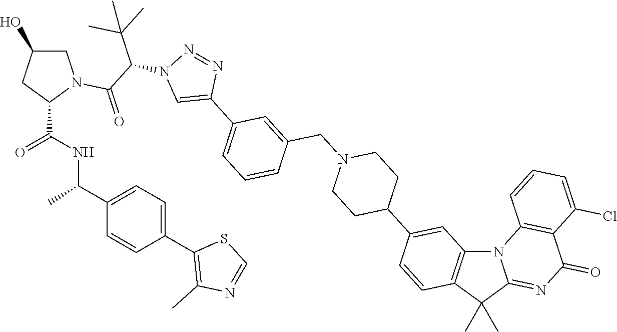 [SMILES: Cc1ncsc1-c1ccc([C@H](C)NC(=O)[C@@H]2C[C@@H](O)CN2C(=O)[C@@H](n2cc(-c3cccc(CN4CCC(c5ccc6c(c5)-n5c(nc(=O)c7c(Cl)cccc75)C6(C)C)CC4)c3)nn2)C(C)(C)C)cc1] | (2S,4R)-1-((S)-2-(4-(3-((4-(4-chloro-7,7- dimethyl-5-oxo-5,7-dihydroindolo[1,2- a]quinazolin-10-yl)piperidin-1- yl)methyl)phenyl)-1H-1,2,3-triazol-1-yl)- 3,3-dimethylbutanoyl)-4-hydroxy-N-((S)- 1-(4-(4-methylthiazol-5- yl)phenyl)ethyl)pyrrolidine-2- carboxamide |
| B120 | 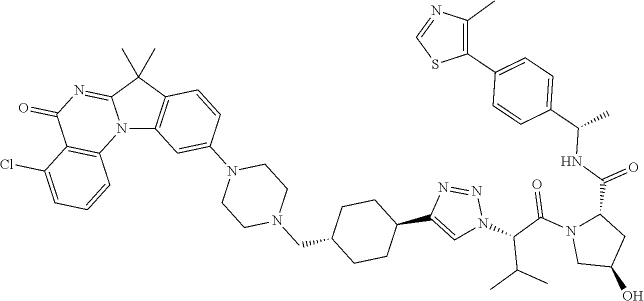 [SMILES: Cc1ncsc1-c1ccc([C@H](C)NC(=O)[C@@H]2C[C@@H](O)CN2C(=O)[C@H](C(C)C)n2cc([C@H]3CC[C@H](CN4CCN(c5ccc6c(c5)-n5c(nc(=O)c7c(Cl)cccc75)C6(C)C)CC4)CC3)nn2)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperazin-1-yl)methyl)cyclohexyl)-1H- 1,2,3-triazol-1-yl)-3-methylbutanoyl)-4- hydroxy-N-((S)-1-(4-(4-methylthiazol-5- yl)phenyl)ethyl)pyrrolidine-2- carboxamide |
| B121 | 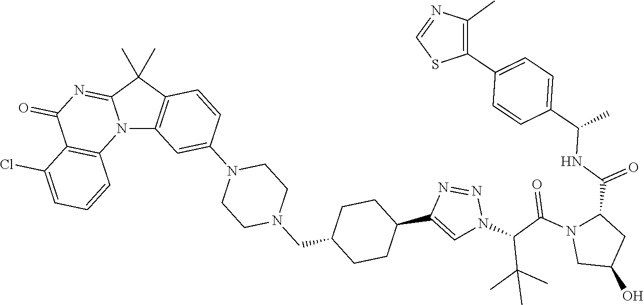 [SMILES: Cc1ncsc1-c1ccc([C@H](C)NC(=O)[C@@H]2C[C@@H](O)CN2C(=O)[C@@H](n2cc([C@H]3CC[C@H](CN4CCN(c5ccc6c(c5)-n5c(nc(=O)c7c(Cl)cccc75)C6(C)C)CC4)CC3)nn2)C(C)(C)C)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperazin-1-yl)methyl)cyclohexyl)-1H- 1,2,3-triazol-1-yl)-3,3-dimethylbutanoyl)- 4-hydroxy-N-((S)-1-(4-(4-methylthiazol- 5-yl)phenyl)ethyl)pyrrolidine-2- carboxamide |
| B122 | 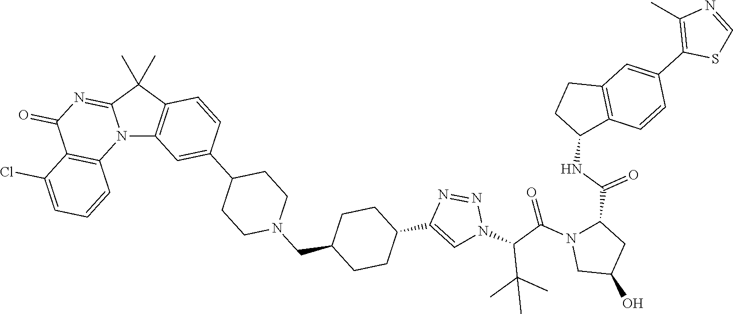 [SMILES: Cc1ncsc1-c1ccc2c(c1)CC[C@H]2NC(=O)[C@@H]1C[C@@H](O)CN1C(=O)[C@@H](n1cc([C@H]2CC[C@H](CN3CCC(c4ccc5c(c4)-n4c(nc(=O)c6c(Cl)cccc64)C5(C)C)CC3)CC2)nn1)C(C)(C)C] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- 4-hydroxy-N-((R)-5-(4-methylthiazol-5- yl)-2,3-dihydro-1H-inden-1- yl)pyrrolidine-2-carboxamide |
| B123 | 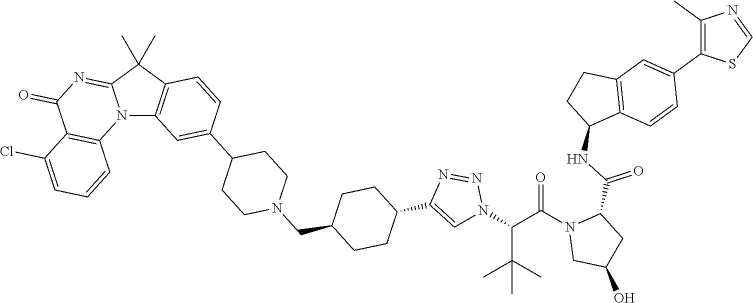 [SMILES: Cc1ncsc1-c1ccc2c(c1)CC[C@@H]2NC(=O)[C@@H]1C[C@@H](O)CN1C(=O)[C@@H](n1cc([C@H]2CC[C@H](CN3CCC(c4ccc5c(c4)-n4c(nc(=O)c6c(Cl)cccc64)C5(C)C)CC3)CC2)nn1)C(C)(C)C] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- 4-hydroxy-N-((S)-5-(4-methylthiazol-5- yl)-2,3-dihydro-1H-inden-1- yl)pyrrolidine-2-carboxamide |
| B124 | 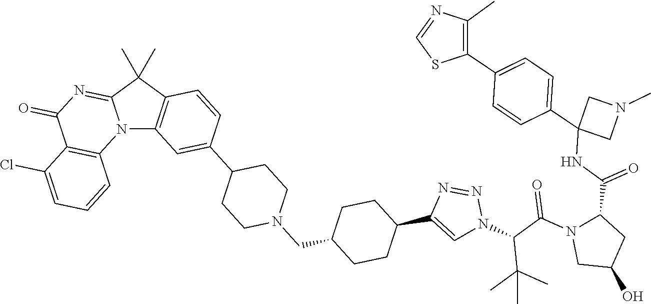 [SMILES: Cc1ncsc1-c1ccc(C2(NC(=O)[C@@H]3C[C@@H](O)CN3C(=O)[C@@H](n3cc([C@H]4CC[C@H](CN5CCC(c6ccc7c(c6)-n6c(nc(=O)c8c(Cl)cccc86)C7(C)C)CC5)CC4)nn3)C(C)(C)C)CN(C)C2)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- 4-hydroxy-N-(1-methyl-3-(4-(4- methylthiazol-5-yl)phenyl)azetidin-3- yl)pyrrolidine-2-carboxamide |
| B125 | 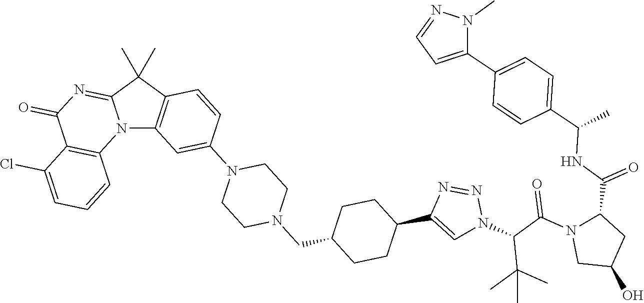 [SMILES: C[C@H](NC(=O)[C@@H]1C[C@@H](O)CN1C(=O)[C@@H](n1cc([C@H]2CC[C@H](CN3CCN(c4ccc5c(c4)-n4c(nc(=O)c6c(Cl)cccc64)C5(C)C)CC3)CC2)nn1)C(C)(C)C)c1ccc(-c2ccnn2C)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperazin-1-yl)methyl)cyclohexyl)-1H- 1,2,3-triazol-1-yl)-3,3-dimethylbutanoyl)- 4-hydroxy-N-((S)-1-(4-(1-methyl-1H- pyrazol-5-yl)phenyl)ethyl)pyrrolidine-2- carboxamide |
| B126 | 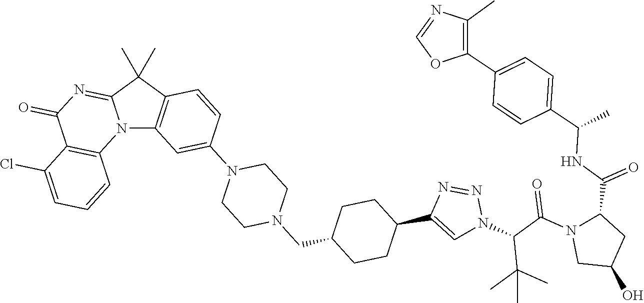 [SMILES: Cc1ncoc1-c1ccc([C@H](C)NC(=O)[C@@H]2C[C@@H](O)CN2C(=O)[C@@H](n2cc([C@H]3CC[C@H](CN4CCN(c5ccc6c(c5)-n5c(nc(=O)c7c(Cl)cccc75)C6(C)C)CC4)CC3)nn2)C(C)(C)C)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperazin-1-yl)methyl)cyclohexyl)-1H- 1,2,3-triazol-1-yl)-3,3-dimethylbutanoyl)- 4-hydroxy-N-((S)-1-(4-(4-methyloxazol- 5-yl)phenyl)ethyl)pyrrolidine-2- carboxamide |
| B127 | 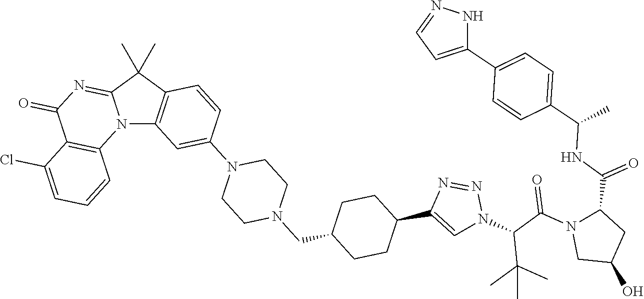 [SMILES: C[C@H](NC(=O)[C@@H]1C[C@@H](O)CN1C(=O)[C@@H](n1cc([C@H]2CC[C@H](CN3CCN(c4ccc5c(c4)-n4c(nc(=O)c6c(Cl)cccc64)C5(C)C)CC3)CC2)nn1)C(C)(C)C)c1ccc(-c2ccn[nH]2)cc1] | (2S,4R)-N-((S)-1-(4-(1H-pyrazol-5- yl)phenyl)ethyl)-1-((S)-2-(4-((1r,4S)-4- ((4-(4-chloro-7,7-dimethyl-5-oxo-5,7- dihydroindolo[1,2-a]quinazolin-10- yl)piperazin-1-yl)methyl)cyclohexyl)-1H- 1,2,3-triazol-1-yl)-3,3-dimethylbutanoyl)- 4-hydroxypyrrolidine-2-carboxamide |
| B128 | 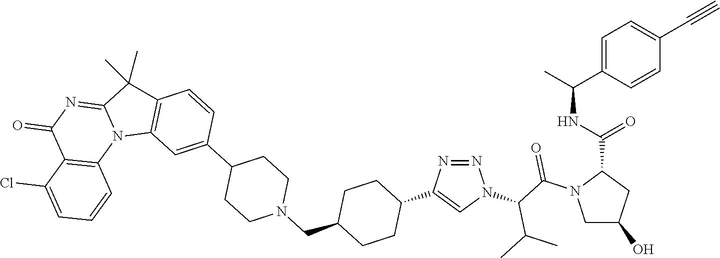 [SMILES: C#Cc1ccc([C@H](C)NC(=O)[C@@H]2C[C@@H](O)CN2C(=O)[C@H](C(C)C)n2cc([C@H]3CC[C@H](CN4CCC(c5ccc6c(c5)-n5c(nc(=O)c7c(Cl)cccc75)C6(C)C)CC4)CC3)nn2)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3-methylbutanoyl)-N- ((S)-1-(4-ethynylphenyl)ethyl)-4- hydroxypyrrolidine-2-carboxamide |
| B129 | 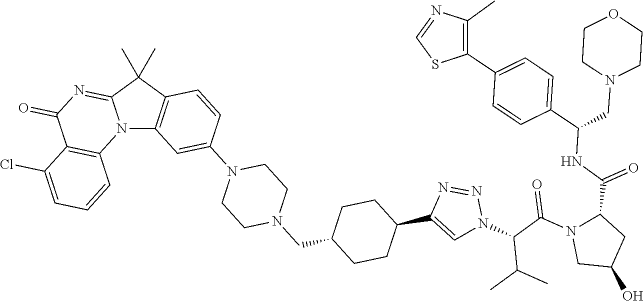 [SMILES: Cc1ncsc1-c1ccc([C@H](CN2CCOCC2)NC(=O)[C@@H]2C[C@@H](O)CN2C(=O)[C@H](C(C)C)n2cc([C@H]3CC[C@H](CN4CCN(c5ccc6c(c5)-n5c(nc(=O)c7c(Cl)cccc75)C6(C)C)CC4)CC3)nn2)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperazin-1-yl)methyl)cyclohexyl)-1H- 1,2,3-triazol-1-yl)-3-methylbutanoyl)-4- hydroxy-N-((R)-1-(4-(4-methylthiazol-5- yl)phenyl)-2- morpholinoethyl)pyrrolidine-2- carboxamide |
| B130 | 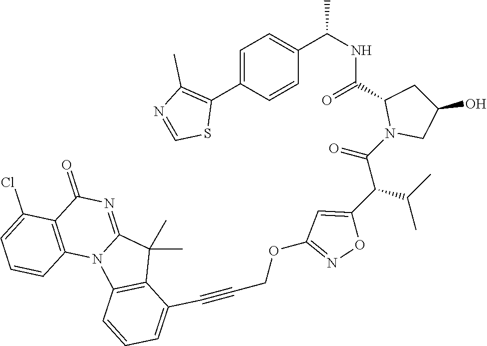 [SMILES: Cc1ncsc1-c1ccc([C@H](C)NC(=O)[C@@H]2C[C@@H](O)CN2C(=O)[C@@H](c2cc(OCC#Cc3cccc4c3C(C)(C)c3nc(=O)c5c(Cl)cccc5n3-4)no2)C(C)C)cc1] | (2S,4R)-1-((R)-2-(3-((3-(4-chloro-7,7- dimethyl-5-oxo-5,7-dihydroindolo[1,2- a]quinazolin-8-yl)prop-2-yn-1- yl)oxy)isoxazol-5-yl)-3-methylbutanoyl)- 4-hydroxy-N-((S)-1-(4-(4-methylthiazol- 5-yl)phenyl)ethyl)pyrrolidine-2- carboxamide |
| B131 | 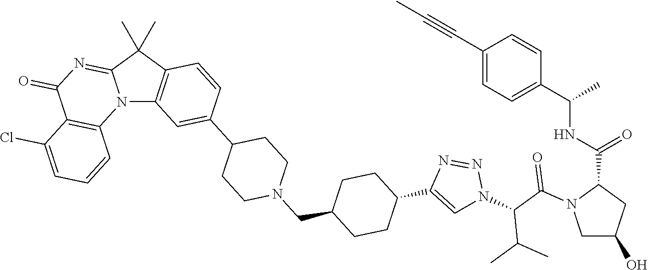 [SMILES: CC#Cc1ccc([C@H](C)NC(=O)[C@@H]2C[C@@H](O)CN2C(=O)[C@H](C(C)C)n2cc([C@H]3CC[C@H](CN4CCC(c5ccc6c(c5)-n5c(nc(=O)c7c(Cl)cccc75)C6(C)C)CC4)CC3)nn2)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3-methylbutanoyl)-4- hydroxy-N-((S)-1-(4-(prop-1-yn-1- yl)phenyl)ethyl)pyrrolidine-2- carboxamide |
| B132 | 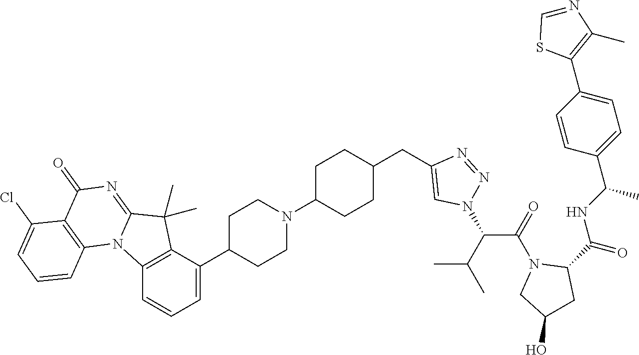 [SMILES: Cc1ncsc1-c1ccc([C@H](C)NC(=O)[C@@H]2C[C@@H](O)CN2C(=O)[C@H](C(C)C)n2cc(CC3CCC(N4CCC(c5cccc6c5C(C)(C)c5nc(=O)c7c(Cl)cccc7n5-6)CC4)CC3)nn2)cc1] | (2S,4R)-1-((S)-2-(4-((4-(4-(4-chloro-7,7- dimethyl-5-oxo-5,7-dihydroindolo[1,2- a]quinazolin-8-yl)piperidin-1- yl)cyclohexyl)methyl)-1H-1,2,3-triazol- 1-yl)-3-methylbutanoyl)-4-hydroxy-N- ((S)-1-(4-(4-methylthiazol-5- yl)phenyl)ethyl)pyrrolidine-2- carboxamide |
| B133 | 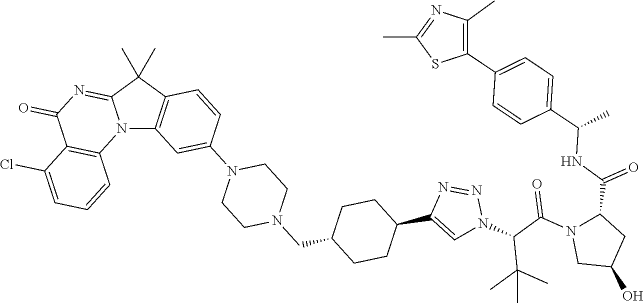 [SMILES: Cc1nc(C)c(-c2ccc([C@H](C)NC(=O)[C@@H]3C[C@@H](O)CN3C(=O)[C@@H](n3cc([C@H]4CC[C@H](CN5CCN(c6ccc7c(c6)-n6c(nc(=O)c8c(Cl)cccc86)C7(C)C)CC5)CC4)nn3)C(C)(C)C)cc2)s1] | (2S,4R)-1-((S)-2-(4-((1r,4S)-4-((4-(4- chloro-7,7-dimethyl-5-oxo-5,7- dihydroindolo[1,2-a]quinazolin-10- yl)piperazin-1-yl)methyl)cyclohexyl)-1H- 1,2,3-triazol-1-yl)-3,3-dimethylbutanoyl)- N-((S)-1-(4-(2,4-dimethylthiazol-5- yl)phenyl)ethyl)-4-hydroxypyrrolidine-2- carboxamide |
| B134 | 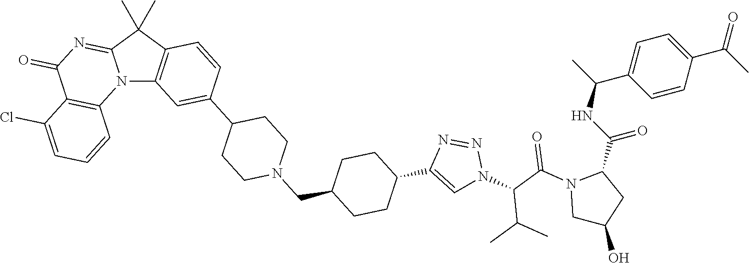 [SMILES: CC(=O)c1ccc([C@H](C)NC(=O)[C@@H]2C[C@@H](O)CN2C(=O)[C@H](C(C)C)n2cc([C@H]3CC[C@H](CN4CCC(c5ccc6c(c5)-n5c(nc(=O)c7c(Cl)cccc75)C6(C)C)CC4)CC3)nn2)cc1] | (2S,4R)-N-((S)-1-(4-acetylphenyl)ethyl)- 1-((S)-2-(4-((1r,4S)-4-((4-(4-chloro-7,7- dimethyl-5-oxo-5,7-dihydroindolo[1,2- a]quinazolin-10-yl)piperidin-1- yl)methyl)cyclohexyl)-1H-1,2,3-triazol- 1-yl)-3-methylbutanoyl)-4- hydroxypyrrolidine-2-carboxamide |
| B135 | 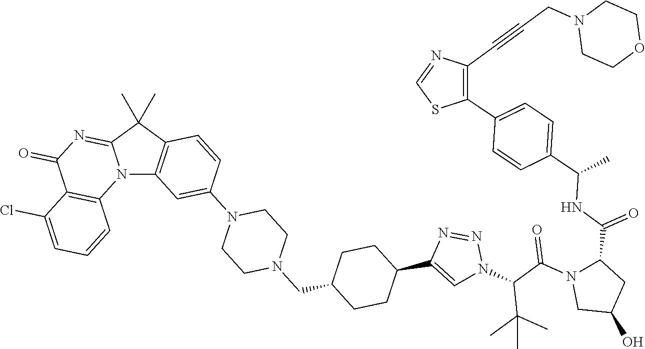 [SMILES: C[C@H](NC(=O)[C@@H]1C[C@@H](O)CN1C(=O)[C@@H](n1cc([C@H]2CC[C@H](CN3CCN(c4ccc5c(c4)-n4c(nc(=O)c6c(Cl)cccc64)C5(C)C)CC3)CC2)nn1)C(C)(C)C)c1ccc(-c2scnc2C#CCN2CCOCC2)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperazin-1-yl)methyl)cyclohexyl)-1H- 1,2,3-triazol-1-yl)-3,3-dimethylbutanoyl)- 4-hydroxy-N-((S)-1-(4-(4-(3- morpholinoprop-1-yn-1-yl)thiazol-5- yl)phenyl)ethyl)pyrrolidine-2- carboxamide |
| B136 | 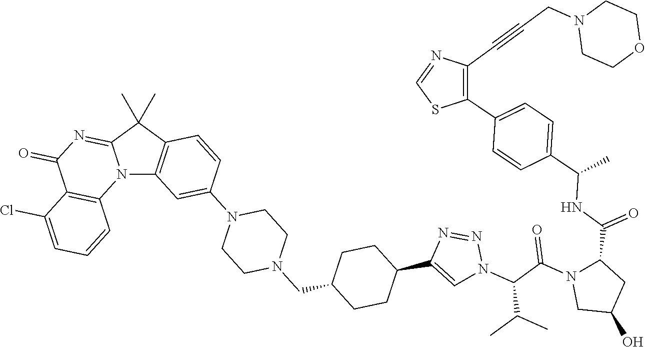 [SMILES: CC(C)[C@@H](C(=O)N1C[C@H](O)C[C@H]1C(=O)N[C@@H](C)c1ccc(-c2scnc2C#CCN2CCOCC2)cc1)n1cc([C@H]2CC[C@H](CN3CCN(c4ccc5c(c4)-n4c(nc(=O)c6c(Cl)cccc64)C5(C)C)CC3)CC2)nn1] | (2S,4R)-1-((S)-2-(4-((1r,4S)-4-((4-(4- chloro-7,7-dimethyl-5-oxo-5,7- dihydroindolo[1,2-a]quinazolin-10- yl)piperazin-1-yl)methyl)cyclohexyl)-1H- 1,2,3-triazol-1-yl)-3-methylbutanoyl)-4- hydroxy-N-((S)-1-(4-(4-(3- morpholinoprop-1-yn-1-yl)thiazol-5- yl)phenyl)ethyl)pyrrolidine-2- carboxamide |
| B137 | 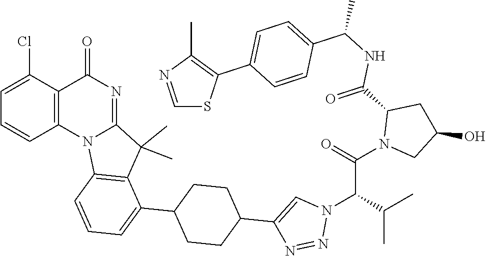 [SMILES: Cc1ncsc1-c1ccc([C@H](C)NC(=O)[C@@H]2C[C@@H](O)CN2C(=O)[C@H](C(C)C)n2cc(C3CCC(c4cccc5c4C(C)(C)c4nc(=O)c6c(Cl)cccc6n4-5)CC3)nn2)cc1] | (2S,4R)-1-((S)-2-(4-(4-(4-chloro-7,7- dimethyl-5-oxo-5,7-dihydroindolo[1,2- a]quinazolin-8-yl)cyclohexyl)-1H-1,2,3- triazol-1-yl)-3-methylbutanoyl)-4- hydroxy-N-((S)-1-(4-(4-methylthiazol-5- yl)phenyl)ethyl)pyrrolidine-2- carboxamide |
| B138 | 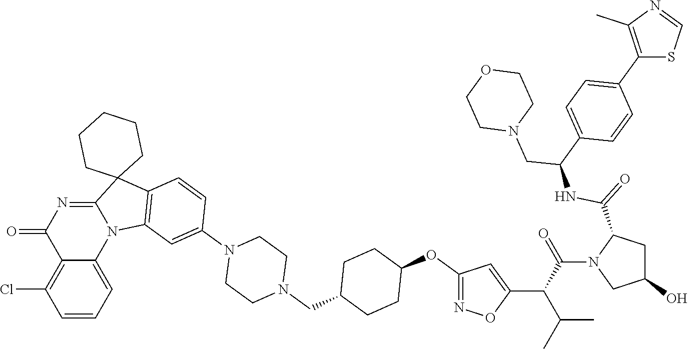 [SMILES: Cc1ncsc1-c1ccc([C@H](CN2CCOCC2)NC(=O)[C@@H]2C[C@@H](O)CN2C(=O)[C@@H](c2cc(O[C@H]3CC[C@H](CN4CCN(c5ccc6c(c5)-n5c(nc(=O)c7c(Cl)cccc75)C65CCCCC5)CC4)CC3)no2)C(C)C)cc1] | (2S,4R)-1-((R)-2-(3-(((1r,4R)-4-((4-(4′- chloro-5′-oxo-5′H-spiro[cyclohexane- 1,7′-indolo[1,2-a]quinazolin]-10′- yl)piperazin-1- yl)methyl)cyclohexyl)oxy)isoxazol-5-yl)- 3-methylbutanoyl)-4-hydroxy-N-((R)-1- (4-(4-methylthiazol-5-yl)phenyl)-2- morpholinoethyl)pyrrolidine-2- carboxamide |
| B139 | 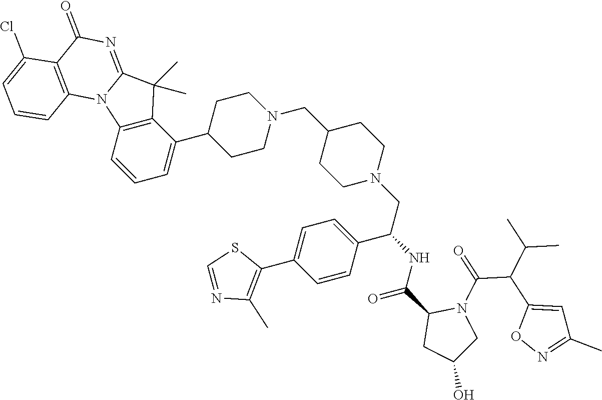 [SMILES: Cc1cc(C(C(=O)N2C[C@H](O)C[C@H]2C(=O)N[C@@H](CN2CCC(CN3CCC(c4cccc5c4C(C)(C)c4nc(=O)c6c(Cl)cccc6n4-5)CC3)CC2)c2ccc(-c3scnc3C)cc2)C(C)C)on1] | (2S,4R)-N-((R)-2-(4-((4-(4-chloro-7,7- dimethyl-5-oxo-5,7-dihydroindolo[1,2- a]quinazolin-8-yl)piperidin-1- yl)methyl)piperidin-1-yl)-1-(4-(4- methylthiazol-5-yl)phenyl)ethyl)-4- hydroxy-1-(3-methyl-2-(3- methylisoxazol-5- yl)butanoyl)pyrrolidine-2-carboxamide |
| B140 | 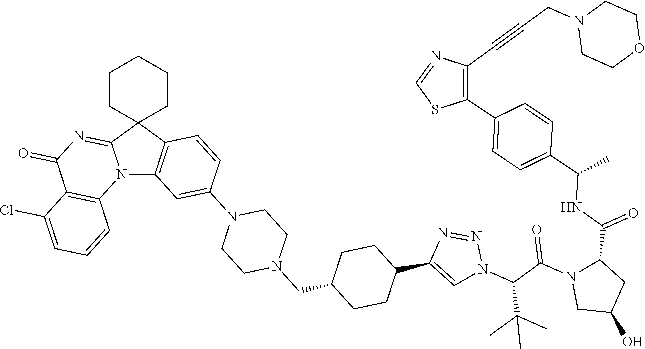 | (2S,4R)-1-((S)-2-(4-((1s,4R)-4-((4-(4′- chloro-5′-oxo-5′H-spiro[cyclohexane- 1,7′-indolo[1,2-a]quinazolin]-10′- yl)piperazin-1-yl)methyl)cyclohexyl)-1H- 1,2,3-triazol-1-yl)-3,3-dimethylbutanoyl)- 4-hydroxy-N-((S)-1-(4-(4-(3- morpholinoprop-1-yn-1-yl)thiazol-5- yl)phenyl)ethyl)pyrrolidine-2- carboxamide |
| B141 | 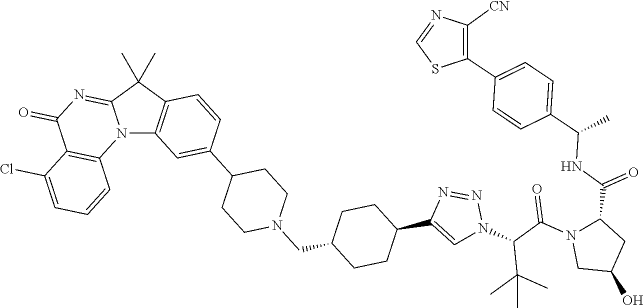 [SMILES: C[C@H](NC(=O)[C@@H]1C[C@@H](O)CN1C(=O)[C@@H](n1cc([C@H]2CC[C@H](CN3CCC(c4ccc5c(c4)-n4c(nc(=O)c6c(Cl)cccc64)C5(C)C)CC3)CC2)nn1)C(C)(C)C)c1ccc(-c2scnc2C#N)cc1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- N-((S)-1-(4-(4-cyanothiazol-5- yl)phenyl)ethyl)-4-hydroxypyrrolidine-2- carboxamide |
| B142 | 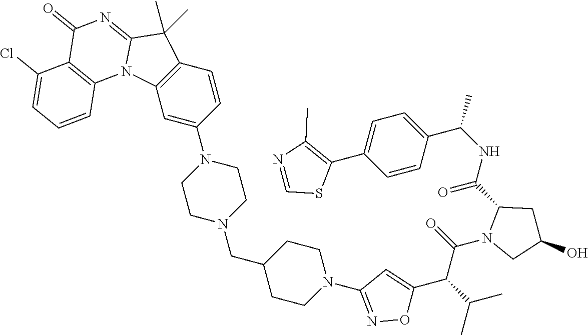 [SMILES: Cc1ncsc1-c1ccc([C@H](C)NC(=O)[C@@H]2C[C@@H](O)CN2C(=O)[C@@H](c2cc(N3CCC(CN4CCN(c5ccc6c(c5)-n5c(nc(=O)c7c(Cl)cccc75)C6(C)C)CC4)CC3)no2)C(C)C)cc1] | (2S,4R)-1-((R)-2-(3-(4-((4-(4-chloro-7,7- dimethyl-5-oxo-5,7-dihydroindolo[1,2- a]quinazolin-10-yl)piperazin-1- yl)methyl)piperidin-1-yl)isoxazol-5-yl)- 3-methylbutanoyl)-4-hydroxy-N-((S)-1- (4-(4-methylthiazol-5- yl)phenyl)ethyl)pyrrolidine-2- carboxamide |
| B143 | 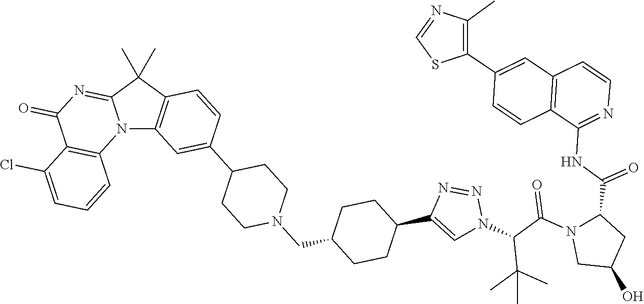 [SMILES: Cc1ncsc1-c1ccc2c(NC(=O)[C@@H]3C[C@@H](O)CN3C(=O)[C@@H](n3cc([C@H]4CC[C@H](CN5CCC(c6ccc7c(c6)-n6c(nc(=O)c8c(Cl)cccc86)C7(C)C)CC5)CC4)nn3)C(C)(C)C)nccc2c1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- 4-hydroxy-N-(6-(4-methylthiazol-5- yl)isoquinolin-1-yl)pyrrolidine-2- carboxamide |
| B144 | 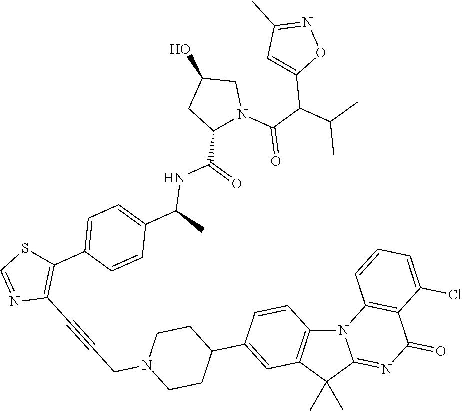 [SMILES: Cc1cc(C(C(=O)N2C[C@H](O)C[C@H]2C(=O)N[C@@H](C)c2ccc(-c3scnc3C#CCN3CCC(c4ccc5c(c4)C(C)(C)c4nc(=O)c6c(Cl)cccc6n4-5)CC3)cc2)C(C)C)on1] | (2S,4R)-N-((S)-1-(4-(4-(3-(4-(4-chloro- 7,7-dimethyl-5-oxo-5,7- dihydroindolo[1,2-a]quinazolin-9- yl)piperidin-1-yl)prop-1-yn-1-yl)thiazol- 5-yl)phenyl)ethyl)-4-hydroxy-1-(3- methyl-2-(3-methylisoxazol-5- yl)butanoyl)pyrrolidine-2-carboxamide |
| B145 | 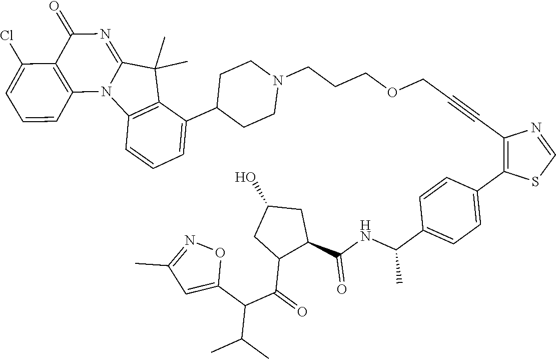 [SMILES: Cc1cc(C(C(=O)C2C[C@H](O)C[C@H]2C(=O)N[C@@H](C)c2ccc(-c3scnc3C#CCOCCCN3CCC(c4cccc5c4C(C)(C)c4nc(=O)c6c(Cl)cccc6n4-5)CC3)cc2)C(C)C)on1] | (2S,4R)-N-((S)-1-(4-(4-(3-(3-(4-(4- chloro-7,7-dimethyl-5-oxo-5,7- dihydroindolo[1,2-a]quinazolin-8- yl)piperidin-1-yl)propoxy)prop-1-yn-1- yl)thiazol-5-yl)phenyl)ethyl)-4-hydroxy- 1-(3-methyl-2-(3-methylisoxazol-5- yl)butanoyl)pyrrolidine-2-carboxamide |
| B146 | 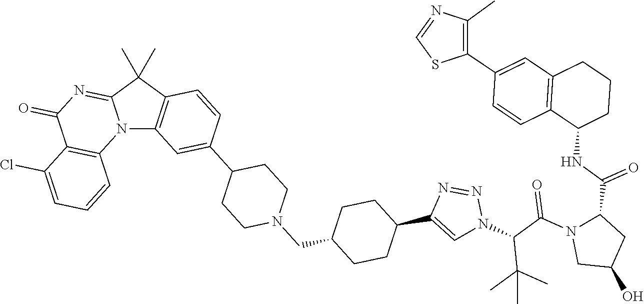 [SMILES: Cc1ncsc1-c1ccc2c(c1)CCC[C@@H]2NC(=O)[C@@H]1C[C@@H](O)CN1C(=O)[C@@H](n1cc([C@H]2CC[C@H](CN3CCC(c4ccc5c(c4)-n4c(nc(=O)c6c(Cl)cccc64)C5(C)C)CC3)CC2)nn1)C(C)(C)C] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- 4-hydroxy-N-((S)-6-(4-methylthiazol-5- yl)-1,2,3,4-tetrahydronaphthalen-1- yl)pyrrolidine-2-carboxamide |
| B147 | 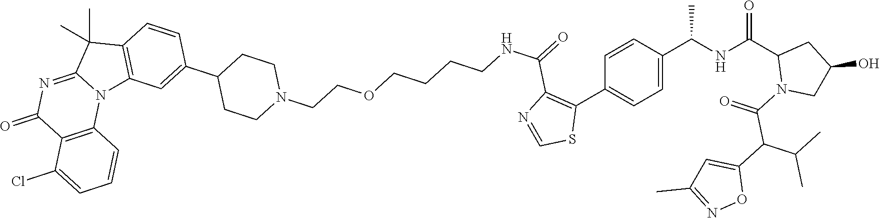 [SMILES: Cc1cc(C(C(=O)N2C[C@H](O)CC2C(=O)N[C@@H](C)c2ccc(-c3scnc3C(=O)NCCCCOCCN3CCC(c4ccc5c(c4)-n4c(nc(=O)c6c(Cl)cccc64)C5(C)C)CC3)cc2)C(C)C)on1] | N-(4-(2-(4-(4-chloro-7,7-dimethyl-5-oxo- 5,7-dihydroindolo[1,2-a]quinazolin-10- yl)piperidin-1-yl)ethoxy)butyl)-5-(4- ((1S)-1-((2S,4R)-4-hydroxy-1-(3-methyl- 2-(3-methylisoxazol-5- yl)butanoyl)pyrrolidine-2- carboxamido)ethyl)phenyl)thiazole-4- carboxamide |
| B148 | 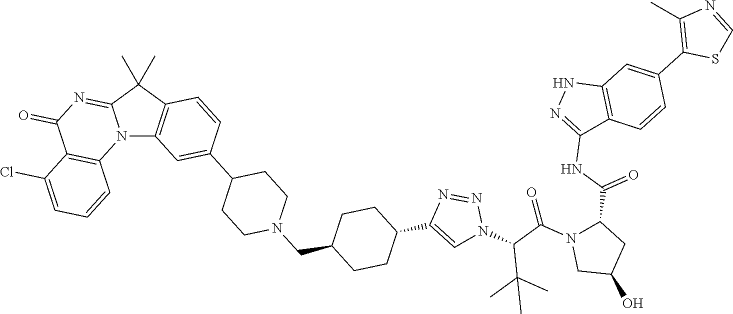 [SMILES: Cc1ncsc1-c1ccc2c(NC(=O)[C@@H]3C[C@@H](O)CN3C(=O)[C@@H](n3cc([C@H]4CC[C@H](CN5CCC(c6ccc7c(c6)-n6c(nc(=O)c8c(Cl)cccc86)C7(C)C)CC5)CC4)nn3)C(C)(C)C)n[nH]c2c1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- 4-hydroxy-N-(6-(4-methylthiazol-5-yl)- 1H-indazol-3-yl)pyrrolidine-2- carboxamide |
| B149 | 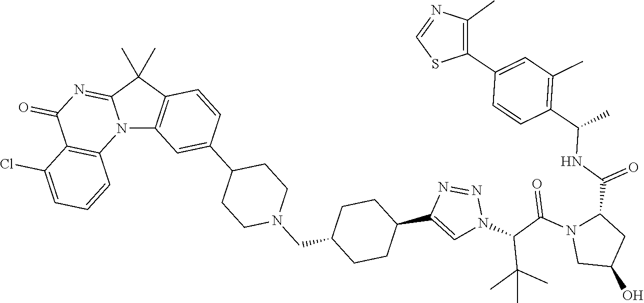 [SMILES: Cc1cc(-c2scnc2C)ccc1[C@H](C)NC(=O)[C@@H]1C[C@@H](O)CN1C(=O)[C@@H](n1cc([C@H]2CC[C@H](CN3CCC(c4ccc5c(c4)-n4c(nc(=O)c6c(Cl)cccc64)C5(C)C)CC3)CC2)nn1)C(C)(C)C] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- 4-hydroxy-N-((S)-1-(2-methyl-4-(4- methylthiazol-5- yl)phenyl)ethyl)pyrrolidine-2- carboxamide |
| B150 | 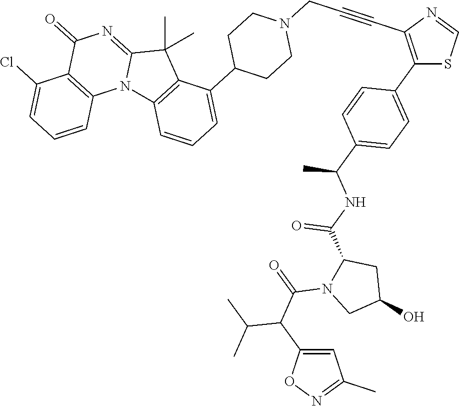 [SMILES: Cc1cc(C(C(=O)N2C[C@H](O)C[C@H]2C(=O)N[C@@H](C)c2ccc(-c3scnc3C#CCN3CCC(c4cccc5c4C(C)(C)c4nc(=O)c6c(Cl)cccc6n4-5)CC3)cc2)C(C)C)on1] | (2S,4R)-N-((S)-1-(4-(4-(3-(4-(4-chloro- 7,7-dimethyl-5-oxo-5,7- dihydroindolo[1,2-a]quinazolin-8- yl)piperidin-1-yl)prop-1-yn-1-yl)thiazol- 5-yl)phenyl)ethyl)-4-hydroxy-1-(3- methyl-2-(3-methylisoxazol-5- yl)butanoyl)pyrrolidine-2-carboxamide |
| B151 | 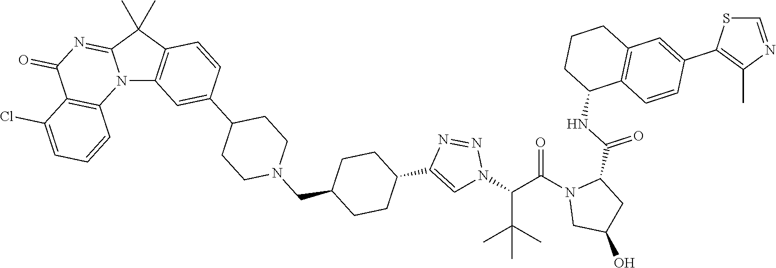 [SMILES: Cc1ncsc1-c1ccc2c(c1)CCC[C@H]2NC(=O)[C@@H]1C[C@@H](O)CN1C(=O)[C@@H](n1cc([C@H]2CC[C@H](CN3CCC(c4ccc5c(c4)-n4c(nc(=O)c6c(Cl)cccc64)C5(C)C)CC3)CC2)nn1)C(C)(C)C] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- 4-hydroxy-N-((R)-6-(4-methylthiazol-5- yl)-1,2,3,4-tetrahydronaphthalen-1- yl)pyrrolidine-2-carboxamide |
| B152 | 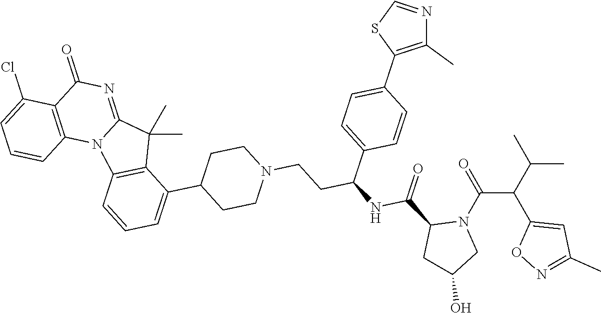 [SMILES: Cc1cc(C(C(=O)N2C[C@H](O)C[C@H]2C(=O)N[C@@H](CCN2CCC(c3cccc4c3C(C)(C)c3nc(=O)c5c(Cl)cccc5n3-4)CC2)c2ccc(-c3scnc3C)cc2)C(C)C)on1] | (2S,4R)-N-((S)-3-(4-(4-chloro-7,7- dimethyl-5-oxo-5,7-dihydroindolo[1,2- a]quinazolin-8-yl)piperidin-1-yl)-1-(4-(4- methylthiazol-5-yl)phenyl)propyl)-4- hydroxy-1-(3-methyl-2-(3- methylisoxazol-5- yl)butanoyl)pyrrolidine-2-carboxamide |
| B153 | 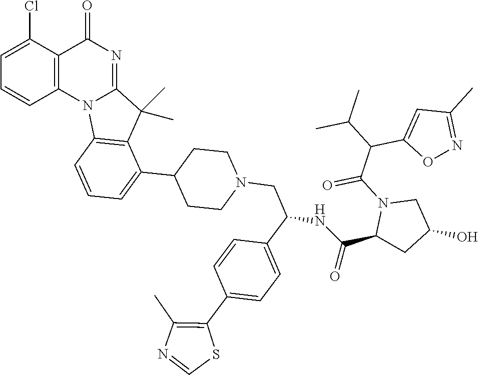 [SMILES: Cc1cc(C(C(=O)N2C[C@H](O)C[C@H]2C(=O)N[C@@H](CN2CCC(c3cccc4c3C(C)(C)c3nc(=O)c5c(Cl)cccc5n3-4)CC2)c2ccc(-c3scnc3C)cc2)C(C)C)on1] | (2S,4R)-N-((R)-2-(4-(4-chloro-7,7- dimethyl-5-oxo-5,7-dihydroindolo[1,2- a]quinazolin-8-yl)piperidin-1-yl)-1-(4-(4- methylthiazol-5-yl)phenyl)ethyl)-4- hydroxy-1-(3-methyl-2-(3- methylisoxazol-5- yl)butanoyl)pyrrolidine-2-carboxamide |
| B154 | 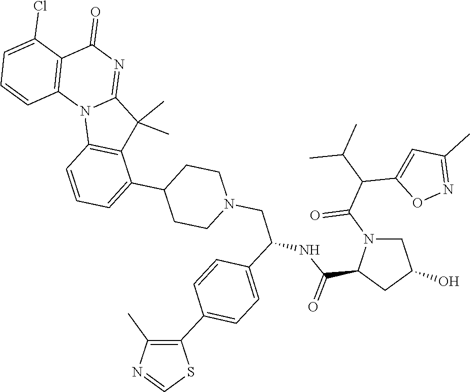 [SMILES: Cc1cc(C(C(=O)N2C[C@H](O)C[C@H]2C(=O)N[C@@H](CN2CCC(c3cccc4c3C(C)(C)c3nc(=O)c5c(Cl)cccc5n3-4)CC2)c2ccc(-c3scnc3C)cc2)C(C)C)on1] | (2S,4R)-N-((R)-2-(4-(4-chloro-7,7- dimethyl-5-oxo-5,7-dihydroindolo[1,2- a]quinazolin-8-yl)piperidin-1-yl)-1-(4-(4- methylthiazol-5-yl)phenyl)ethyl)-4- hydroxy-1-(3-methyl-2-(3- methylisoxazol-5- yl)butanoyl)pyrrolidine-2-carboxamide |
| B155 | 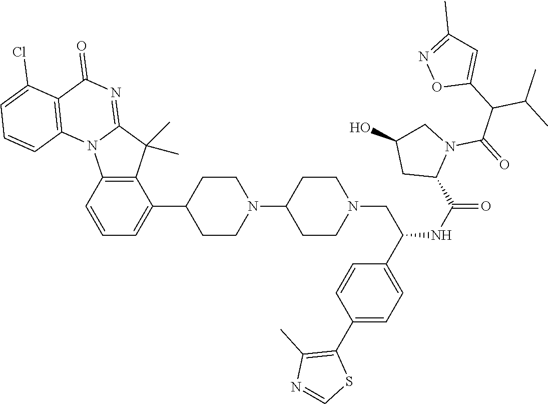 [SMILES: Cc1cc(C(C(=O)N2C[C@H](O)C[C@H]2C(=O)N[C@@H](CN2CCC(N3CCC(c4cccc5c4C(C)(C)c4nc(=O)c6c(Cl)cccc6n4-5)CC3)CC2)c2ccc(-c3scnc3C)cc2)C(C)C)on1] | (2S,4R)-N-((R)-2-(4-(4-chloro-7,7- dimethyl-5-oxo-5,7-dihydroindolo[1,2- a]quinazolin-8-yl)-[1,4'-bipiperidin]-1'- yl)-1-(4-(4-methylthiazol-5- yl)phenyl)ethyl)-4-hydroxy-1-(3-methyl- 2-(3-methylisoxazol-5- yl)butanoyl)pyrrolidine-2-carboxamide |
| B156 | 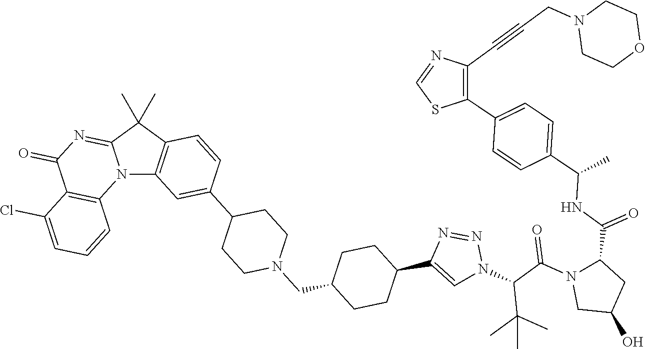 [SMILES: C[C@H](NC(=O)[C@@H]1C[C@@H](O)CN1C(=O)[C@@H](n1cc([C@H]2CC[C@H](CN3CCC(c4ccc5c(c4)-n4c(nc(=O)c6c(Cl)cccc64)C5(C)C)CC3)CC2)nn1)C(C)(C)C)c1ccc(-c2scnc2C#CCN2CCOCC2)cc1] | (2S,4R)-1-((R)-2-(4-((1r,4R)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- 4-hydroxy-N-((S)-1-(4-(4-(3- morpholinoprop-1-yn-1-yl)thiazol-5- yl)phenyl)ethyl)pyrrolidine-2- carboxamide |
| B157 | 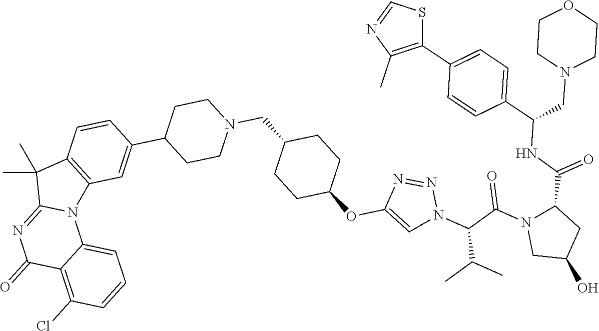 [SMILES: Cc1ncsc1-c1ccc([C@H](CN2CCOCC2)NC(=O)[C@@H]2C[C@@H](O)CN2C(=O)[C@H](C(C)C)n2cc(O[C@H]3CC[C@H](CN4CCC(c5ccc6c(c5)-n5c(nc(=O)c7c(Cl)cccc75)C6(C)C)CC4)CC3)nn2)cc1] | (2S,4R)-1-((R)-2-(3-(((1r,4R)-4-((4-(4- chloro-7,7-dimethyl-5-oxo-5,7- dihydroindolo[1,2-a]quinazolin-10- yl)piperidin-1- yl)methyl)cyclohexyl)oxy)isoxazol-5-yl)- 3-methylbutanoyl)-4-hydroxy-N-((R)-1- (4-(4-methylthiazol-5-yl)phenyl)-2- morpholinoethyl)pyrrolidine-2- carboxamide |
| B158 | 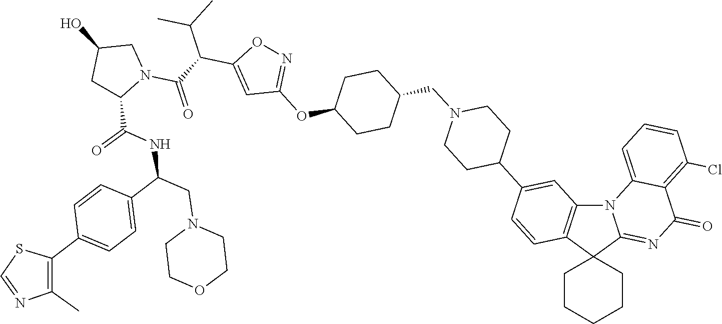 [SMILES: Cc1ncsc1-c1ccc([C@H](CN2CCOCC2)NC(=O)[C@@H]2C[C@@H](O)CN2C(=O)[C@@H](c2cc(O[C@H]3CC[C@H](CN4CCC(c5ccc6c(c5)-n5c(nc(=O)c7c(Cl)cccc75)C65CCCCC5)CC4)CC3)no2)C(C)C)cc1] | (2S,4R)-1-((R)-2-(3-(((11,4R)-4-((4-(4′- chloro-5′-oxo-5′H-spiro[cyclohexane- 1,7′-indolo[1,2-a]quinazolin]-10′- yl)piperidin-1- yl)methyl)cyclohexyl)oxy)isoxazol-5-yl)- 3-methylbutanoyl)-4-hydroxy-N-((R)-1- (4-(4-methylthiazol-5-yl)phenyl)-2- morpholinoethyl)pyrrolidine-2- carboxamide |
| B159 | 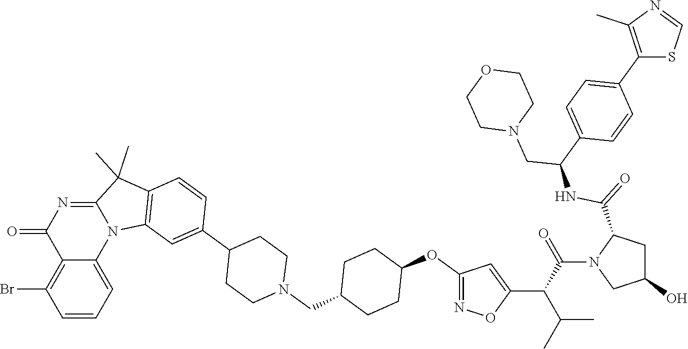 [SMILES: Cc1ncsc1-c1ccc([C@H](CN2CCOCC2)NC(=O)[C@@H]2C[C@@H](O)CN2C(=O)[C@@H](c2cc(O[C@H]3CC[C@H](CN4CCC(c5ccc6c(c5)-n5c(nc(=O)c7c(Br)cccc75)C6(C)C)CC4)CC3)no2)C(C)C)cc1] | (2S,4R)-1-((R)-2-(3-(((1r,4R)-4-((4-(4′- bromo-5′-oxo-5′H-spiro[cyclohexane- 1,7′-indolo[1,2-a]quinazolin]-10′- yl)piperidin-1- yl)methyl)cyclohexyl)oxy)isoxazol-5-yl)- 3-methylbutanoyl)-4-hydroxy-N-((R)-1- (4-(4-methylthiazol-5-yl)phenyl)-2- morpholinoethyl)pyrrolidine-2- carboxamide |
| B160 | 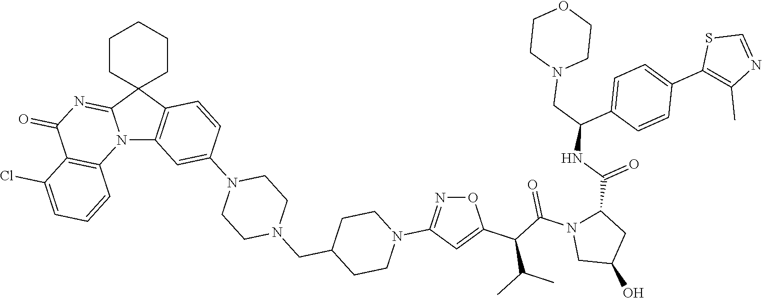 [SMILES: Cc1ncsc1-c1ccc([C@H](CN2CCOCC2)NC(=O)[C@@H]2C[C@@H](O)CN2C(=O)[C@H](c2cc(N3CCC(CN4CCN(c5ccc6c(c5)-n5c(nc(=O)c7c(Cl)cccc75)C65CCCCC5)CC4)CC3)no2)C(C)C)cc1] | (2S,4R)-1-((S)-2-(3-(4-((4-(4′-chloro-5′- oxo-5′H-spiro[cyclohexane-1,7′- indolo[1,2-a]quinazolin]-10′-yl)piperazin- 1-yl)methyl)piperidin-1-yl)isoxazol-5- yl)-3-methylbutanoyl)-4-hydroxy-N-((S)- 1-(4-(4-methylthiazol-5-yl)phenyl)-2- morpholinoethyl)pyrrolidine-2- carboxamide |
| B161 | 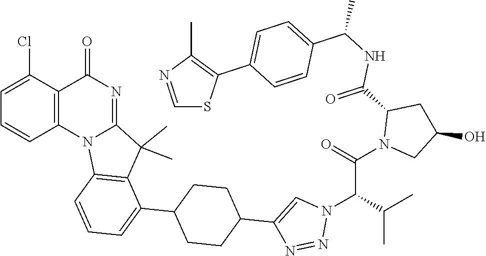 [SMILES: Cc1ncsc1-c1ccc([C@H](C)NC(=O)[C@@H]2C[C@@H](O)CN2C(=O)[C@H](C(C)C)n2cc(C3CCC(c4cccc5c4C(C)(C)c4nc(=O)c6c(Cl)cccc6n4-5)CC3)nn2)cc1] | (2S,4R)-1-((S)-2-(4-(4-(4-chloro-7,7- dimethyl-5-oxo-5,7-dihydroindolo[1,2- a]quinazolin-8-yl)cyclohexyl)-1H-1,2,3- triazol-1-yl)-3-methylbutanoyl)-4- hydroxy-N-((S)-1-(4-(4-methylthiazol-5- yl)phenyl)ethyl)pyrrolidine-2- carboxamide |
| B162 | 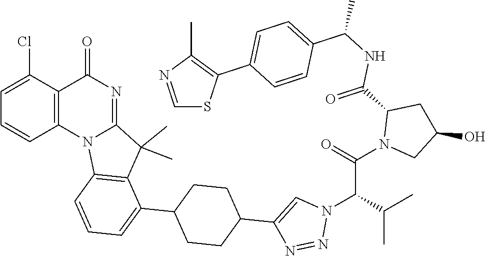 [SMILES: Cc1ncsc1-c1ccc([C@H](C)NC(=O)[C@@H]2C[C@@H](O)CN2C(=O)[C@H](C(C)C)n2cc(C3CCC(c4cccc5c4C(C)(C)c4nc(=O)c6c(Cl)cccc6n4-5)CC3)nn2)cc1] | (2S,4R)-1-((S)-2-(4-(4-(4-chloro-7,7- dimethyl-5-oxo-5,7-dihydroindolo[1,2- a]quinazolin-8-yl)cyclohexyl)-1H-1,2,3- triazol-1-yl)-3-methylbutanoyl)-4- hydroxy-N-((S)-1-(4-(4-methylthiazol-5- yl)phenyl)ethyl)pyrrolidine-2- carboxamide |
| B163 | 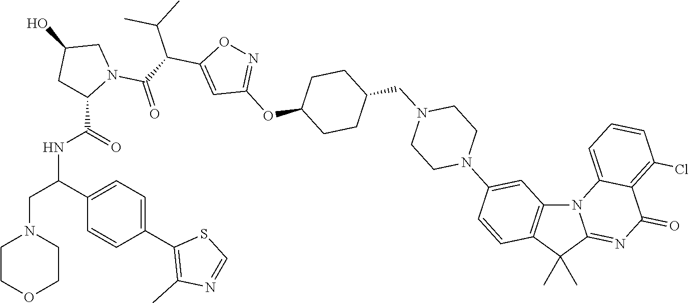 [SMILES: Cc1ncsc1-c1ccc(C(CN2CCOCC2)NC(=O)[C@@H]2C[C@@H](O)CN2C(=O)[C@@H](c2cc(O[C@H]3CC[C@H](CN4CCN(c5ccc6c(c5)-n5c(nc(=O)c7c(Cl)cccc75)C6(C)C)CC4)CC3)no2)C(C)C)cc1] | (2S,4R)-1-((R)-2-(3-(((11,4R)-4-((4-(4- chloro-7,7-dimethyl-5-oxo-5,7- dihydroindolo[1,2-a]quinazolin-10- yl)piperazin-1- yl)methyl)cyclohexyl)oxy)isoxazol-5-yl)- 3-methylbutanoyl)-4-hydroxy-N-((R)-1- (4-(4-methylthiazol-5-yl)phenyl)-2- morpholinoethyl)pyrrolidine-2- carboxamide |
| B164 | 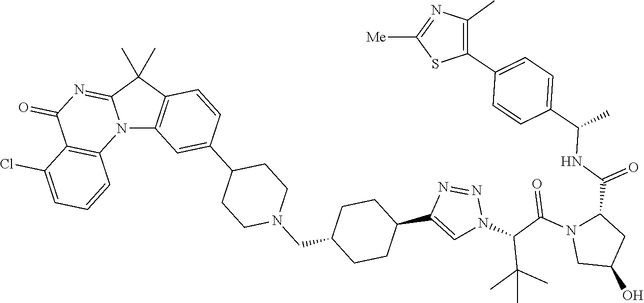 [SMILES: Cc1nc(C)c(-c2ccc([C@H](C)NC(=O)[C@@H]3C[C@@H](O)CN3C(=O)[C@@H](n3cc([C@H]4CC[C@H](CN5CCC(c6ccc7c(c6)-n6c(nc(=O)c8c(Cl)cccc86)C7(C)C)CC5)CC4)nn3)C(C)(C)C)cc2)s1] | (2S,4R)-1-((S)-2-(4-((1r,4S)-4-((4-(4- chloro-7,7-dimethyl-5-oxo-5,7- dihydroindolo[1,2-a]quinazolin-10- yl)piperidin-1-yl)methyl)cyclohexyl)-1H- 1,2,3-triazol-1-yl)-3,3-dimethylbutanoyl)- N-((S)-1-(4-(2,4-dimethylthiazol-5- yl)phenyl)ethyl)-4-hydroxypyrrolidine-2- carboxamide |
| B165 | 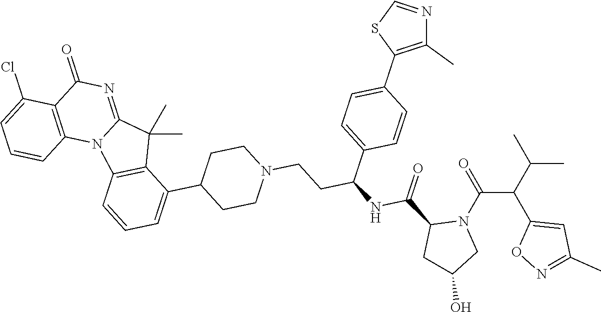 [SMILES: Cc1cc(C(C(=O)N2C[C@H](O)C[C@H]2C(=O)N[C@@H](CCN2CCC(c3cccc4c3C(C)(C)c3nc(=O)c5c(Cl)cccc5n3-4)CC2)c2ccc(-c3scnc3C)cc2)C(C)C)on1] | (2S,4R)-N-((S)-3-(4-(4-chloro-7,7- dimethyl-5-oxo-5,7-dihydroindolo[1,2- a]quinazolin-8-yl)piperidin-1-yl)-1-(4-(4- methylthiazol-5-yl)phenyl)propyl)-4- hydroxy-1-(3-methyl-2-(3- methylisoxazol-5- yl)butanoyl)pyrrolidine-2-carboxamide |
| B166 | 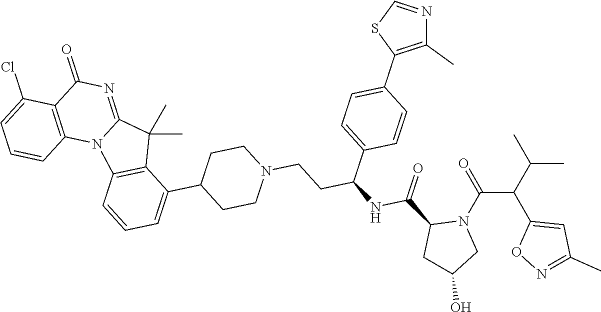 [SMILES: Cc1cc(C(C(=O)N2C[C@H](O)C[C@H]2C(=O)N[C@@H](CCN2CCC(c3cccc4c3C(C)(C)c3nc(=O)c5c(Cl)cccc5n3-4)CC2)c2ccc(-c3scnc3C)cc2)C(C)C)on1] | (2S,4R)-N-((S)-3-(4-(4-chloro-7,7- dimethyl-5-oxo-5,7-dihydroindolo[1,2- a]quinazolin-8-yl)piperidin-1-yl)-1-(4-(4- methylthiazol-5-yl)phenyl)propyl)-4- hydroxy-1-(3-methyl-2-(3- methylisoxazol-5- yl)butanoyl)pyrrolidine-2-carboxamide |
| B167 | 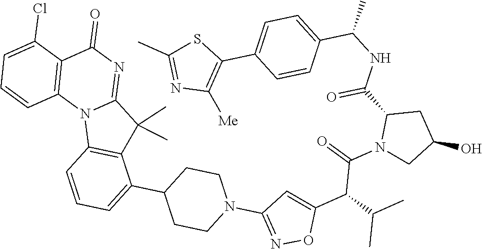 [SMILES: Cc1nc(C)c(-c2ccc([C@H](C)NC(=O)[C@@H]3C[C@@H](O)CN3C(=O)[C@@H](c3cc(N4CCC(c5cccc6c5C(C)(C)c5nc(=O)c7c(Cl)cccc7n5-6)CC4)no3)C(C)C)cc2)s1] | (2S,4R)-1-(2-(3-(4-(4-chloro-7,7- dimethyl-5-oxo-5,7-dihydroindolo[1,2- a]quinazolin-8-yl)piperidin-1-yl)isoxazol- 5-yl)-3-methylbutanoyl)-N-((S)-1-(4- (2,4-dimethylthiazol-5-yl)phenyl)ethyl)- 4-hydroxypyrrolidine-2-carboxamide |
| B168 | 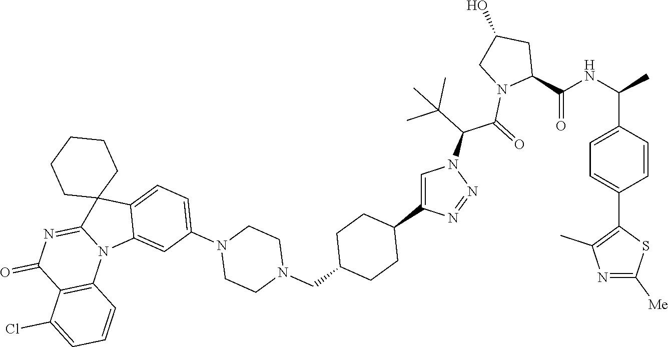 [SMILES: Cc1nc(C)c(-c2ccc([C@H](C)NC(=O)[C@@H]3C[C@@H](O)CN3C(=O)[C@@H](n3cc([C@H]4CC[C@H](CN5CCN(c6ccc7c(c6)-n6c(nc(=O)c8c(Cl)cccc86)C76CCCCC6)CC5)CC4)nn3)C(C)(C)C)cc2)s1] | (2S,4R)-1-((S)-2-(4-((1,4S)-4-((4-(4′- chloro-5′-oxo-5′H-spiro[cyclohexane- 1,7′-indolo[1,2-a]quinazolin]-10′- yl)piperazin-1-yl)methyl)cyclohexyl)-1H- 1,2,3-triazol-1-yl)-3,3-dimethylbutanoyl)- N-((S)-1-(4-(2,4-dimethylthiazol-5- yl)phenyl)ethyl)-4-hydroxypyrrolidine-2- carboxamide |
| B169 | 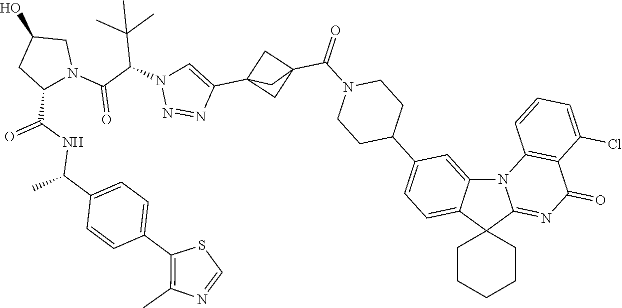 [SMILES: Cc1ncsc1-c1ccc([C@H](C)NC(=O)[C@@H]2C[C@@H](O)CN2C(=O)[C@@H](n2cc(C34CC(C(=O)N5CCC(c6ccc7c(c6)-n6c(nc(=O)c8c(Cl)cccc86)C76CCCCC6)CC5)(C3)C4)nn2)C(C)(C)C)cc1] | (2S,4R)-1-((S)-2-(4-(3-(4-(4′-chloro-5′- oxo-5′H-spiro[cyclohexane-1,7′- indolo[1,2-a]quinazolin]-10′- yl)piperidine-1- carbonyl)bicyclo[1.1.1]pentan-1-yl)-1H- 1,2,3-triazol-1-yl)-3,3-dimethylbutanoyl)- 4-hydroxy-N-((S)-1-(4-(4-methylthiazol- 5-yl)phenyl)ethyl)pyrrolidine-2- carboxamide |

The compounds of the present disclosure may possess advantageous characteristics, as compared to known compounds, such as known SMARCA2/4 degraders. For example, the compounds of the present disclosure may display more SMARCA2/4 activity, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction). These beneficial properties of the compounds of the present disclosure can be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In one embodiment, a compound of the present disclosure (e.g., a compound of any of the formulae or any individual compounds disclosed herein) is a pharmaceutically acceptable salt. In another embodiment, a compound of the present disclosure (e.g., a compound of any of the formulae or any individual compounds disclosed herein) is a solvate. In another embodiment, a compound of the present disclosure (e.g., a compound of any of the formulae or any individual compounds disclosed herein) is a hydrate.

### Pharmaceutically Acceptable Salts

In certain embodiments, the compounds disclosed herein exist as their pharmaceutically acceptable salts. In certain embodiments, the methods disclosed herein include methods of treating diseases by administering such pharmaceutically acceptable salts. In certain embodiments, the methods disclosed herein include methods of treating diseases by administering such pharmaceutically acceptable salts as pharmaceutical compositions.

In certain embodiments, the compounds described herein possess acidic or basic groups and therefor react with any of a number of inorganic or organic bases, and inorganic and organic acids, to form a pharmaceutically acceptable salt. In certain embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In certain embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In certain embodiments, water or oil-soluble or dispersible products are obtained by such quaternization.

### Solvates

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the disclosure may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the disclosure.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present disclosure.

In certain embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

Isomers (stereoisomers, geometric isomer, tautomer, etc.)

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is termed a “racemic mixture”.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight (S)-compound, at least about 99% by weight (R)-compound and at most about 1% by weight (S)-compound, or at least about 99.9% by weight (R)-compound and at most about 0.1% by weight (S)-compound. In certain embodiments, the weights are based upon total weight of compound.

As used herein and unless otherwise indicated, the term “enantiomerically pure(S)-compound” refers to at least about 95% by weight (S)-compound and at most about 5% by weight (R)-compound, at least about 99% by weight (S)-compound and at most about 1% by weight (R)-compound or at least about 99.9% by weight (S)-compound and at most about 0.1% by weight (R)-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In certain embodiments, the enantiomerically pure (R)-compound in such compositions can, for example, comprise, at least about 95% by weight (R)-compound and at most about 5% by weight (S)-compound, by total weight of the compound. For example, a pharmaceutical composition comprising enantiomerically pure(S)-compound can comprise, for example, about 90% excipient and about 10% enantiomerically pure(S)-compound. In certain embodiments, the enantiomerically pure(S)-compound in such compositions can, for example, comprise, at least about 95% by weight (S)-compound and at most about 5% by weight (R)-compound, by total weight of the compound. In certain embodiments, the active ingredient can be formulated with little or no excipient or carrier.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

In certain embodiments, the compounds described herein exist as geometric isomers. In certain embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the disclosure.

In certain embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the disclosure.

In additional embodiments of the compounds and methods provided herein, mixtures of enantiomers and/or diastereoisomers, resulting from a single preparative step, combination, or interconversion are useful for the applications described herein. In certain embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In certain embodiments, dissociable complexes are preferred. In certain embodiments, the diastereomers have distinct physical properties (e.g., melting points, boiling points, solubilities, reactivity, etc.) and are separated by taking advantage of these dissimilarities. In certain embodiments, the diastereomers are separated by chiral chromatography, or preferably, by separation/resolution techniques based upon differences in solubility. In certain embodiments, the optically pure enantiomer is then recovered, along with the resolving agent.

### Tautomers

In certain embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest. All tautomeric forms of the compounds disclosed herein are contemplated and are within the scope of the disclosure. The exact ratio of the tautomers depends on several factors, including temperature, solvent, and pH.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound described herein, or a pharmaceutically acceptable salt, solvate, or stereoisomer thereof, and a pharmaceutically acceptable excipient.

In certain embodiments, the compound provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

### Preparation and Characterization of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., Examples).

### General Synthetic Method

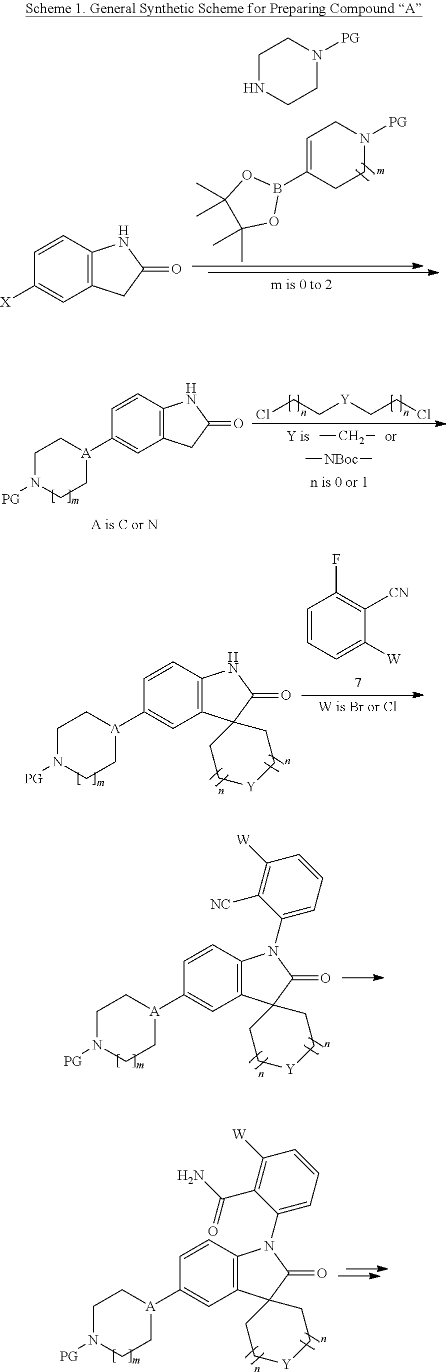

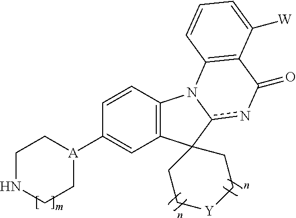

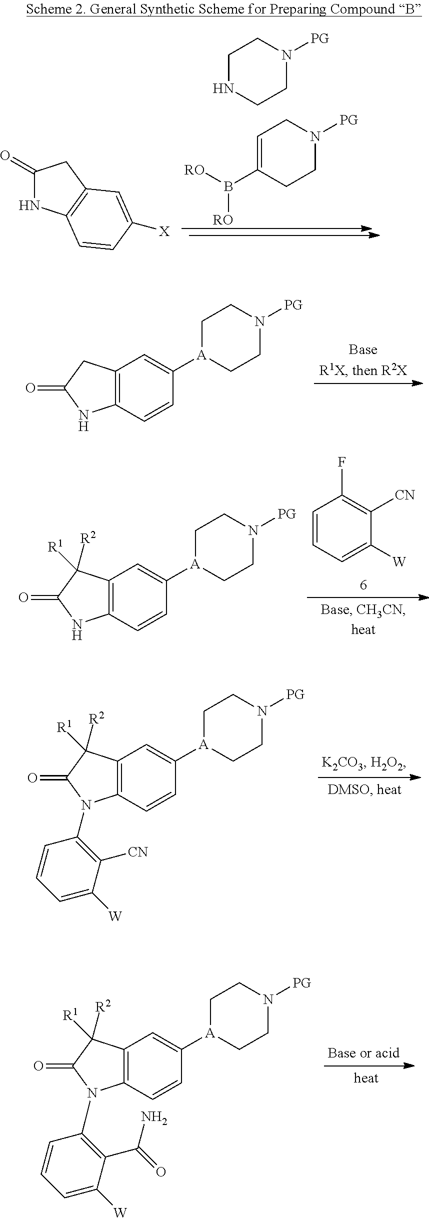

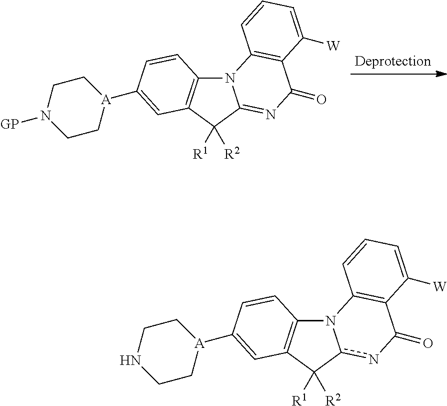

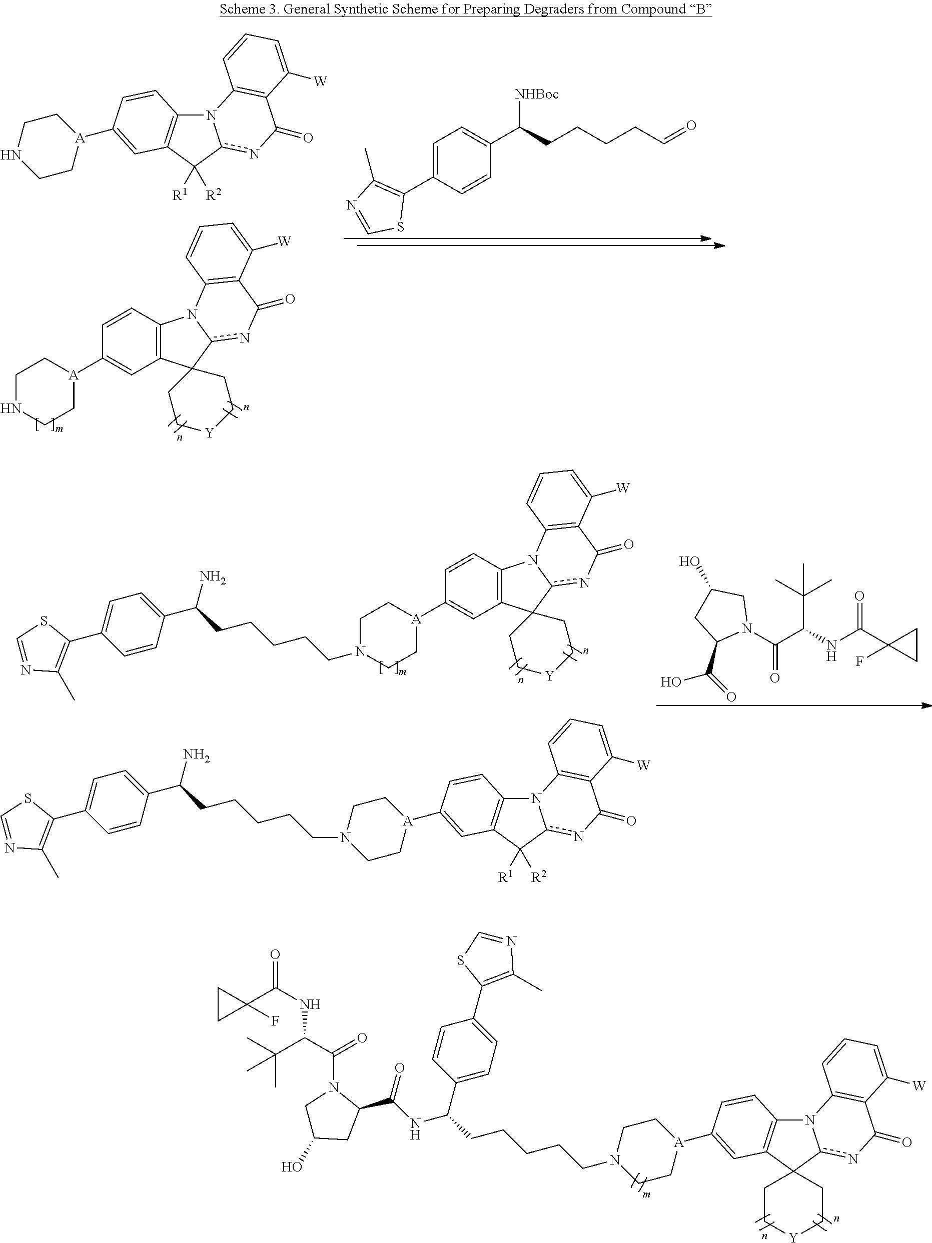

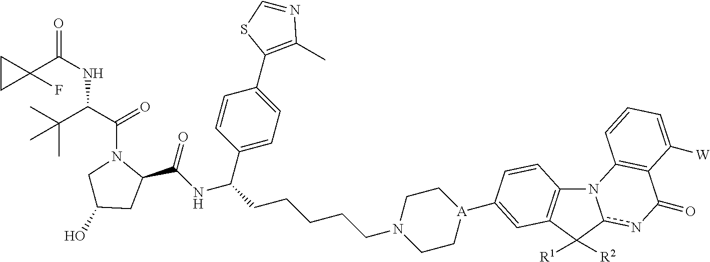

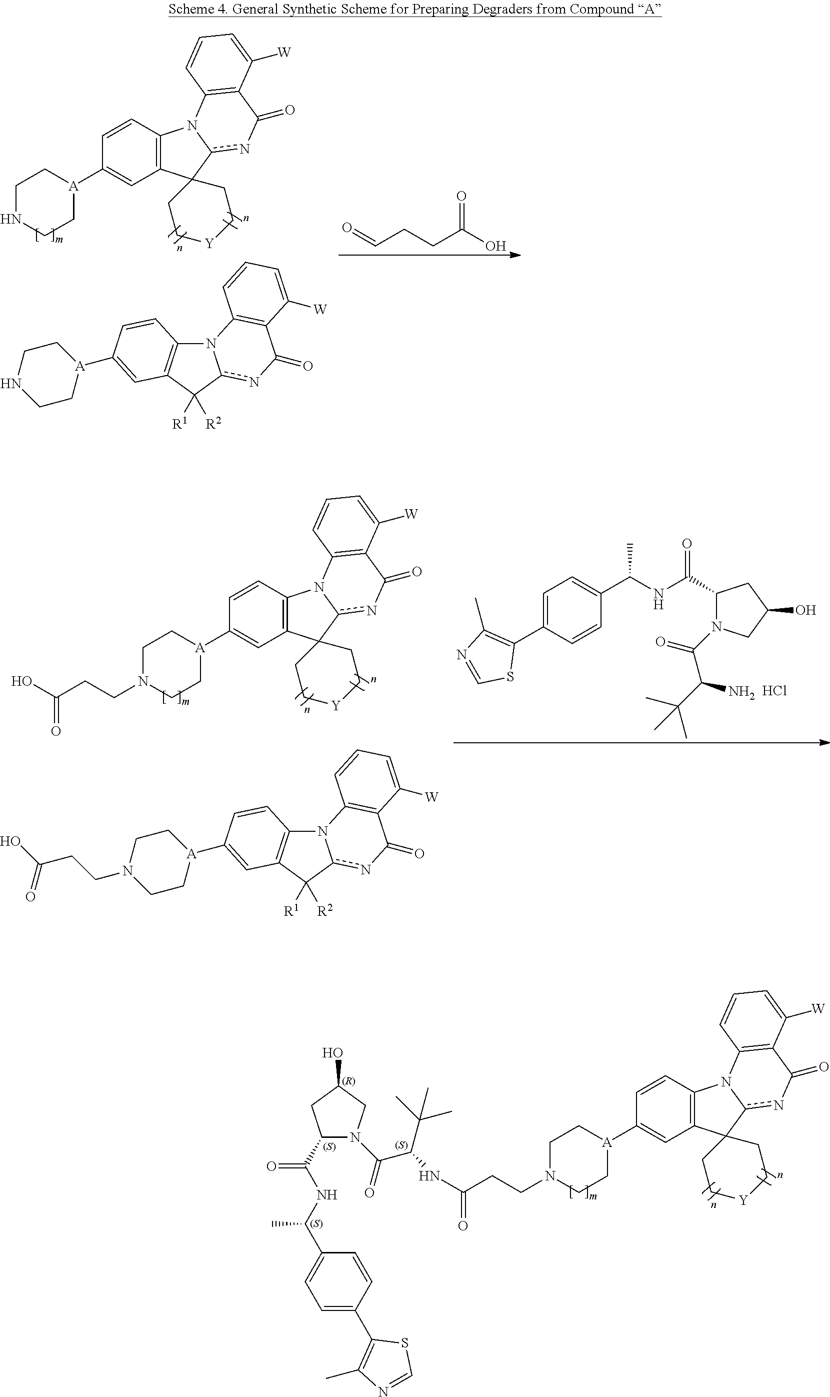

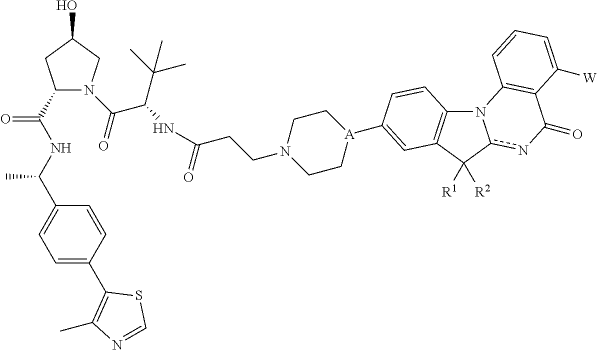

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 400 MHz. Spectra are given in ppm (δ) and coupling constants, J, are reported in Hertz. Tetramethylsilane (TMS) was used as an internal standard. Liquid chromatography-mass spectrometry (LC/MS) were collected using a SHIMADZU LCMS-2020 EV or Agilent 1260-6125B LCMS. Purity and low-resolution mass spectral data were measured using Agilent 1260-6125B LCMS system (with Diode Array Detector, and Agilent G6125BA Mass spectrometer) or using Waters Acquity UPLC system (with Diode Array Detector, and Waters 3100 Mass Detector). The purity was characterized by UV wavelength 214 nm, 220 nm, 254 nm and ESI. Column: poroshell 120 EC-C18 2.7 μm 4.6×100 mm; Flow rate 0.8 mL/min; Solvent A (100/0.1 water/formic acid), Solvent B (100 acetonitrile); gradient: hold 5% B to 0.3 min, 5-95% B from 0.3 to 2 min, hold 95% B to 4.8 min, 95-5% B from 4.8 to 5.4 min, then hold 5% B to 6.5 min. Or, column: Acquity UPLC BEH C18 1.7 μm 2.1×50 mm; Flow rate 0.5 mL/min; Solvent A (0.1% formic acid water), Solvent B (acetonitrile); gradient: hold 5% B for 0.2 min, 5-95% B from 0.2 to 2.0 min, hold 95% B to 3.1 min, then 5% B at 3.5 min.

### Biological Assays

The biological activities of the compounds of the present application can be assessed with methods and assays known in the art.

In certain embodiments, protein degradation is measured using HiBiT Assay. In a typical HiBiT assay, Hela cells are genetically modified via CRISPR/Cas9 to fuse HiBiT to the carboxy terminus of SMARCA2 (Promega CS302365) or SMARCA4 (Promega CS3023225). Cells are cultured in a certain media (e.g., DMEM containing 10% FBS and 1% (Vol:Vol) penicillin-streptomycin). At the time of assay, cells are seeded at certain dentistry (e.g., 20,000 cells per well) and subsequently treated with serial dilutions of testing compounds for a certain period of time (e.g., 24 hours). Levels of SMARCA2 or SMARCA4 expression are assessed, e.g., with Nano-Glo® HiBiT Lytic Detection Assay (Promega N3050). IC₅₀ is obtained, e.g., using the GraphPad Prism data analysis or other appropriate software.

The HiBit assay may also be performed using engineered HT1080 CRISPR knock-in cell. In a typical assay, cells are seeded onto serially diluted compounds at a certain density (e.g., 2000 cells/25 uL/well) and incubated for a certain period of time (e.g., 6 hrs) in a tissue culture incubator at a certain temperature (e.g., 37° C.) and a certain concentration of CO₂ (e.g., 5%). The LgBiT Protein is diluted (e.g., at 1:100) and the Nano-Glo® HiBIT Lytic Substrate (e.g., 1:50) into an appropriate volume of room temperature using, e.g., the Nano-Glo® HiBiT Lytic Buffer. At the end of incubation time, detection reagent is added to each well (without LgBiT for negative control wells) (e.g., using the Thermo Scientific multidrop combi). The plate is shaken for a certain period of time (e.g., 10 mins) at a certain temperature (e.g., RT). After brief centrifugation (e.g., 2000 rpm for 1 mins), plate is read on a reader (e.g., Envision reader (Ultrasensitive luminescence model)).

### Methods of Use

In certain aspects, the present disclosure provides methods of degrading a SMARCA2 or SMARCA4 protein in a subject, comprising administering to the subject a compound disclosed herein.

In certain aspects, the present disclosure provides uses of a compound disclosed herein in the manufacture of a medicament for degrading a SMARCA2 or SMARCA4 protein in a subject.

In certain aspects, the present disclosure provides compounds disclosed herein for use in degrading a SMARCA2 or SMARCA4 protein in a subject.

In certain aspects, the present disclosure provides methods of treating or preventing a disease or disorder in a subject in need thereof, comprising administering to the subject a compound disclosed herein (e.g., in a therapeutically effective amount).

In certain aspects, the present disclosure provides methods of treating a disease or disorder in a subject in need thereof, comprising administering to the subject a compound disclosed herein (e.g., in a therapeutically effective amount).

In certain aspects, the present disclosure provides uses of a compound disclosed herein in the manufacture of a medicament for treating or preventing a disease or disorder in a subject in need thereof.

In certain aspects, the present disclosure provides uses of a compound disclosed herein in the manufacture of a medicament for treating a disease or disorder in a subject in need thereof.

In certain aspects, the present disclosure provides compounds disclosed herein for use in treating or preventing a disease or disorder in a subject in need thereof.

In certain aspects, the present disclosure provides compounds disclosed herein for use in treating a disease or disorder in a subject in need thereof.

In certain embodiments, the disease or disorder is a SMARCA2 or SMARCA4 protein-mediated disease or disorder.

In certain embodiments, the disease or disorder is cancer.

In certain embodiments, the cancer is non-small cell lung cancer, small-cell lung cancer, colorectal cancer, bladder cancer, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

In certain embodiments, the cancer is selected from NSCLC adenocarcinoma (LUAD), NSCL squamous cell carcinoma (LUSC), liver hepatocellular carcinoma (LIHC), uterine corpus endometrial carcinoma (UCEC), esophageal carcinoma (ESCA), skin cutaneous melanoma (SKCM), stomach adenocarcinoma (STAD), colon adenocarcinoma (COAD), bladder urothelial carcinoma (BLCA), and uterine carcinosarcoma (UCS).

In certain embodiments, the cancer is selected from NSCLC adenocarcinoma (LUAD), NSCL squamous cell carcinoma (LUSC), liver hepatocellular carcinoma (LIHC), and uterine corpus endometrial carcinoma (UCEC).

In certain embodiments, the cancer includes, but is not limited to, one or more of the cancers of Table A.

TABLE A

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic fibroma |
| lymphoma | rhabdomyosarcoma | sarcoma | |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial tumor | |
| embryonal carcinoma | endocrine gland | endodermal sinus | enteropathy- |
| | neoplasm | tumor | associated T-cell |
| | | | lymphoma |
| esophageal cancer | fetus in fetu | fibroma | fibrosarcoma |
| follicular lymphoma | follicular thyroid | ganglioneuroma | gastrointestinal |
| | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck cancer | hemangiopericytoma | hematological cancer | hepatoblastoma |
| hepatosplenic T-cell | Hodgkin's lymphoma | non-Hodgkin's | invasive lobular |
| lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung cancer | non-small cell lung | MALT lymphoma |
| | | cancer | |
| malignant fibrous | malignant peripheral | malignant triton | mantle cell |
| histiocytoma | nerve sheath tumor | tumor | lymphoma |
| marginal zone B-cell | mast cell leukemia | mediastinal germ cell | medullary carcinoma |
| lymphoma | | tumor | of the breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid liposarcoma | myxoma | myxosarcoma | nasopharyngeal |
| | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal zone | squamous cell | synovial sarcoma | Sezary's disease |
| lymphoma | carcinoma | | |
| small intestine cancer | squamous carcinoma | stomach cancer | T-cell lymphoma |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In certain embodiments, the cancer is a solid tumor. In certain embodiments, the cancer is a hematological cancer. Exemplary hematological cancers include, but are not limited to, the cancers listed in Table B. In certain embodiments, the hematological cancer is acute lymphocytic leukemia, chronic lymphocytic leukemia (including B-cell chronic lymphocytic leukemia), or acute myeloid leukemia.

TABLE B

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic lymphoma |
| Waldenstrom's macroglobulinemia (WM) | T-cell lymphoma |
| diffuse large B-cell lymphoma (DLBCL) | mast cell leukemia |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell lymphoma |
| Richter's transformation | |

In certain embodiments, the subject is a mammal.

In certain embodiments, the subject is a human.

### EXEMPLARY EMBODIMENTS

### Exemplary Embodiment 1

A compound of Formula II

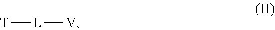

or a pharmaceutically acceptable salt, solvate, or stereoisomer thereof, wherein:
T is of Formula II-1

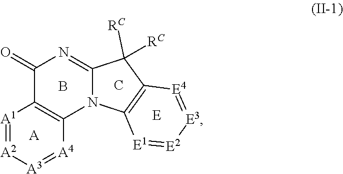

wherein:
A¹ is CR^{A1} or N;
A² is CR^{A2} or N;
A³ is CR^{A3} or N;
A⁴ is CR^{A4} or N;
R^{A1}, R^{A2}, R^{A3}, and R^{A4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂ORŁ, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{a}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two R^{C} together form an oxo; or
two R^{C}, together with the carbon atom to which they are attached, form Ring D

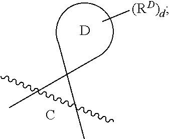

Ring D is C₃₋₁₂ carbocycle or 3- to 12-membered heterocycle;
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR′, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 10, as valency permits;
E¹ is CR^{E1} or N;
E² is CR^{E2} or N;
E³ is CR^{E3} or N;
E⁴ is CR^{E4} or N;

R^{E1}, R^{E2}, R^{E3}, and R^{E4} are independently

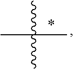

hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O) NR^{c}R^{d}, wherein the alkyl, alkoxy, alkylamino, alkenyl, alkynyl, carbocyclyl, heterocyclyl, aryl, or heteroaryl is optionally substituted with one or more R^{u};
   wherein one of R^{E1}, R^{E2}, R^{E3}, and R^{E4} is

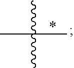

or
one of R^{E2}, R^{E3}, or R^{E4} is

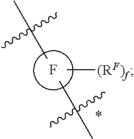

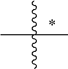

denotes attachment to L;
Ring F is C₃₋₁₂ carbocyclyl, 3- to 12-membered heterocyclyl, C₆₋₁₀ aryl, or 5- to 10-membered heteroaryl;
each R^{F} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
f is an integer selected from 0 to 10, as valency permits,
L is of Formula II-2

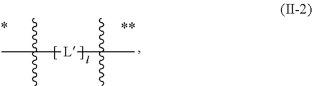

wherein:
* denotes attachment to T and ** denotes attachment to V;
each L′ is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O) N(R^{L′})—, —C(═O)O—, —N(R^{L′})—, —O—, —S—, or —S(═O)₂—, wherein the alkylene, heteroalkylene, alkenylene, alkynylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
l is an integer selected from 0 to 5,
   V is of Formula II-3-i, II-3-ii, II-3-iii, II-3-iv, II-3-v, II-3-vi, II-3-vii, or II-3-viii

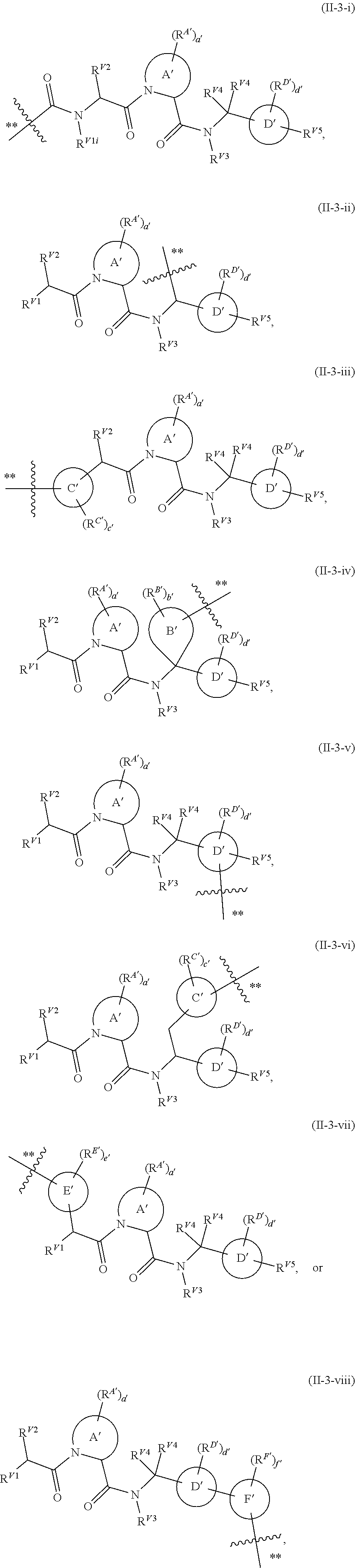

wherein:
** denotes attachment to L;
Ring A′ is 5- to 7-membered heterocycle;
each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₁₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a′ is an integer selected from 0 to 10, as valency permits;
Ring B′ is C₃₋₆ carbocycle or 3- to 6-membered heterocycle;
each R^{B′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
b′ is an integer selected from 0 to 10, as valency permits;
Ring C′ is C₆ aryl or 5- to 6-membered heteroaryl;
each R^{C′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
c′ is an integer selected from 0 to 5, as valency permits;
Ring D′ is C₆ aryl or 5- to 6-membered heteroaryl;
each R^{D′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
R^{D′} and R^{V4}, together with the intervening atoms, form C₃₋₁₂ carbocyclyl, 3- to 12-membered heterocyclyl, C₆₋₁₀ aryl, or 5- to 10-membered heteroaryl, wherein the carbocyclyl, heterocyclyl, aryl, or heteroaryl is optionally substituted with one or more R^{u};
d′ is an integer selected from 0 to 5, as valency permits; Ring E′ is C₃₋₈ carbocycle or 3- to 8-membered heterocycle;
each R^{E′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
e′ is an integer selected from 0 to 5, as valency permits;
Ring F′ is C₆ aryl or 5- to 6-membered heteroaryl;
each R^{F′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
f is an integer selected from 0 to 5, as valency permits;
R^{V1} is —N(R^{V1i})C(═O)R^{V1ii} or 5- to 6-membered heteroaryl optionally substituted with one or more R^{u};
R^{V1i} is hydrogen, C₁₋₆ alkyl, C₃₋₆ carbocyclyl, or 3- to 6-membered heterocyclyl, wherein the alkyl, carbocyclyl, or heterocyclyl is optionally substituted with one or more R^{u};

R^{V1ii} is C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{V1iia};
each R^{V1iia} is independently oxo, halogen, —CN, —NO₂, —OH, —NH₂, C₁₋₆ alkyl, —(C₁₋₆ alkylene)-(3- to 12-membered heterocyclyl), C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkylene, alkoxy, alkylamino, alkenyl, alkynyl, carbocyclyl, heterocyclyl, aryl, or heteroaryl is optionally substituted with one or more R^{u};
R^{V2} is hydrogen, C₁₋₆ alkyl, C₃₋₁₂ carbocyclyl, or 3- to 12-membered heterocyclyl, wherein the alkyl, carbocyclyl, heterocyclyl is optionally substituted with one or more R^{u};
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O) NR^{c}R^{d}, wherein the alkyl, carbocyclyl, or heterocyclyl is optionally substituted with one or more R^{u};
each R^{V4} is independently hydrogen, C₁₋₆ alkyl, C₂₋₆ alkenyl, C₂₋₆ alkynyl, C₃₋₆ carbocyclyl, or 3- to 6-membered heterocyclyl, wherein the alkyl, alkenyl, alkynyl, carbocyclyl, or heterocyclyl is optionally substituted with one or more R^{V4a}; or
two R^{V4}, together with the carbon atom to which they are attached, form C₃₋₆ carbocycle or 3- to 6-membered heterocycle, wherein the carbocycle or heterocycle is optionally substituted with one or more R^{u};
each R^{V4a} is independently oxo, halogen, —CN, —NO₂, —OH, —NH₂, C₁₋₆ alkyl, C₁₋₁₂ heteroalkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, heteroalkyl, alkoxy, alkylamino, alkenyl, alkynyl, carbocyclyl, heterocyclyl, aryl, or heteroaryl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, or —C(═O)R^{a}, wherein the alkyl, alkoxy, alkylamino, alkenyl, alkynyl, carbocyclyl, heterocyclyl, aryl, or heteroaryl is optionally substituted with one or more R^{u};
   wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₃₋₆ carbocyclyl, 3- to 6-membered heterocyclyl, C₆ aryl, or 5- to 6-membered heteroaryl, wherein the carbocyclyl, heterocyclyl, aryl, or heteroaryl is optionally substituted with one or more R⁷;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl, wherein each occurrence of R^{a}, R^{b}, R^{c}, and R^{d} is independently and optionally substituted with one or more R²; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

### Exemplary Embodiment 2

The compound of any one of the preceding Exemplary Embodiments, wherein T is of Formula II-1-i or II-1-ii

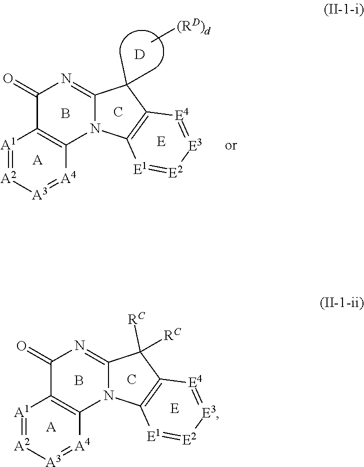

wherein:
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

### Exemplary Embodiment 3

The compound of any one of the preceding Exemplary Embodiments, wherein T is of Formula II-1-i-1, II-1-i-2, II-1-i-3, II-1-ii-1, II-1-ii-2, II-1-ii-3, II-1-iii-1, II-1-iii-2, or II-1-iii-3

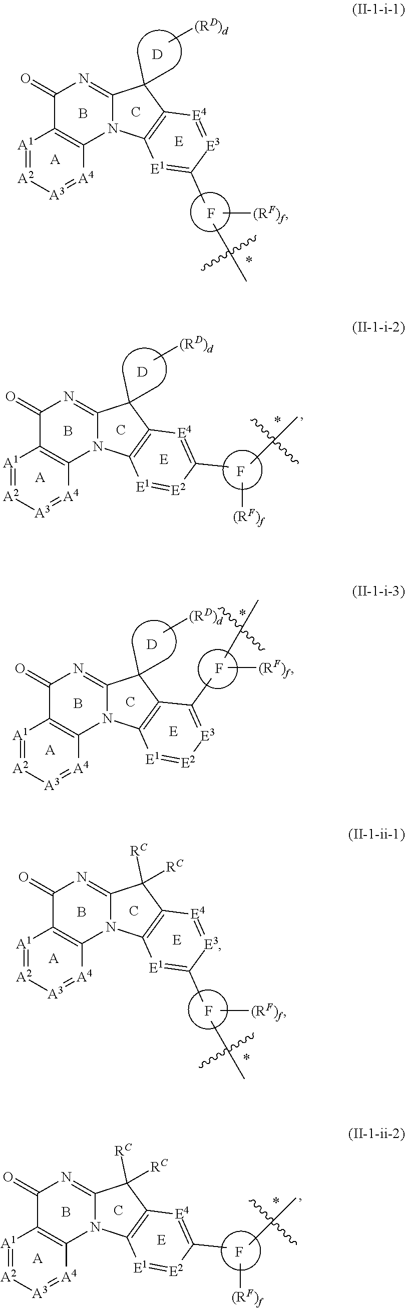

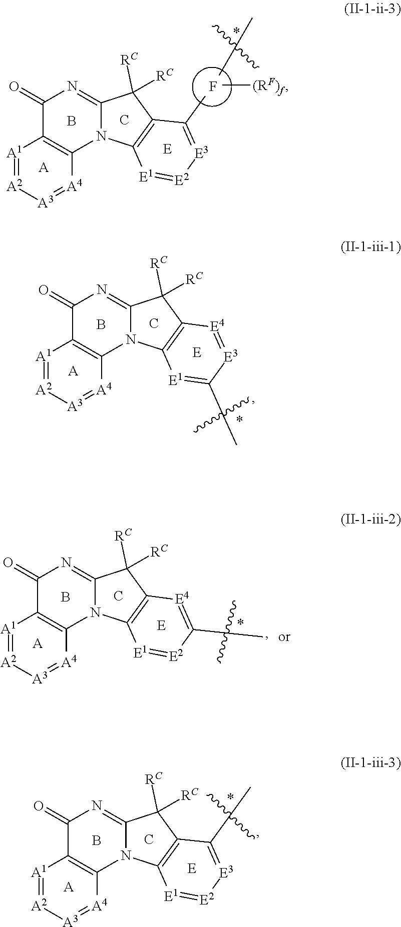

each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

### Exemplary Embodiment 4

The compound of any one of the preceding Exemplary Embodiments, wherein R^{A1} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

### Exemplary Embodiment 5

The compound of any one of the preceding Exemplary Embodiments, wherein R^{A1} is halogen or hydrogen.

### Exemplary Embodiment 6

The compound of any one of the preceding Exemplary Embodiments, wherein T is of Formula II-1-i-4, II-1-i-5, II-1-i-6, II-1-ii-4, II-1-ii-5, II-1-ii-6, II-1-iii-4, II-1-iii-5, or II-1-iii-6

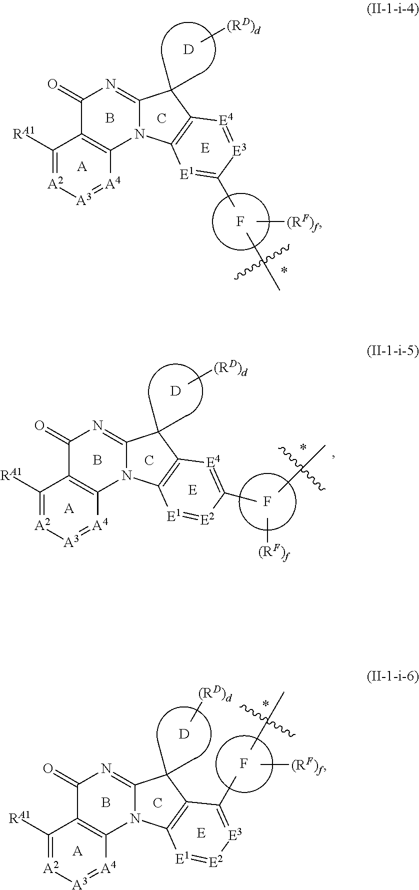

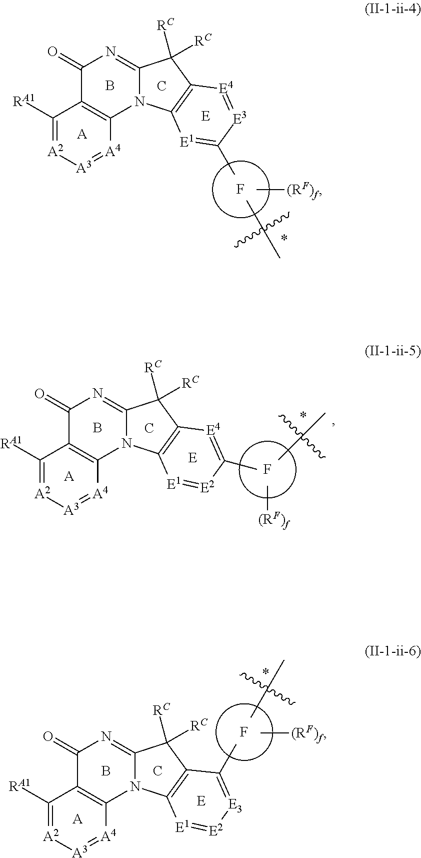

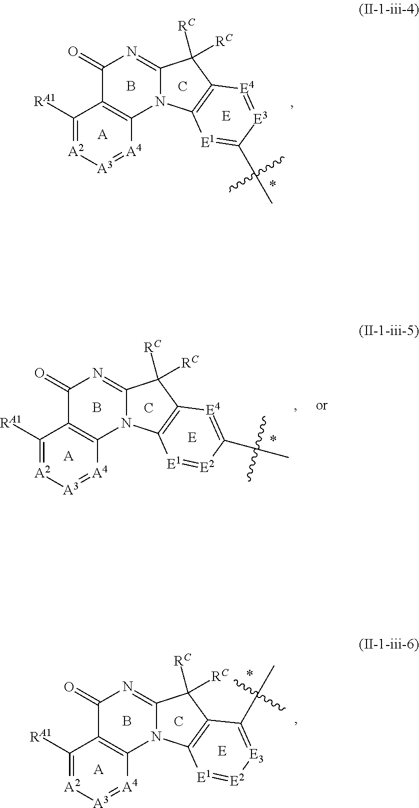

wherein
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; and
R^{A1} is halogen or hydrogen.

### Exemplary Embodiment 7

The compound of any one of the preceding Exemplary Embodiments, wherein Ring F is 3- to 12-membered heterocyclyl or C₃₋₁₂ carbocyclyl.

### Exemplary Embodiment 8

The compound of any one of the preceding Exemplary Embodiments, wherein Ring F is 5- to 6-membered heteroaryl.

### Exemplary Embodiment 9

The compound of any one of the preceding Exemplary Embodiments, wherein each R^{F} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

### Exemplary Embodiment 10

The compound of any one of the preceding Exemplary Embodiments, wherein f is 0.

### Exemplary Embodiment 11

The compound of any one of the preceding Exemplary Embodiments, wherein R^{E1}, R^{E2}, and R^{E4}, R^{E1}, R^{E3}, and R^{E4}, or R^{E1}, R^{E2}, and R^{E3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

### Exemplary Embodiment 12

The compound of any one of the preceding Exemplary Embodiments, wherein each of R^{E1}, R^{E2}, and R^{E4}, each of R^{E1}, R^{E3}, and R^{E4}, or each of R^{E1}, R^{E2}, and R^{E3} is hydrogen.

### Exemplary Embodiment 13

The compound of any one of the preceding Exemplary Embodiments, wherein one or two of E¹, E², and E⁴, one or two of E¹, E³, and E⁴, or one or two of E¹, E², and E³ are N.

### Exemplary Embodiment 14

The compound of any one of the preceding Exemplary Embodiments, wherein one or two of R^{E1}, R^{E2}, and R^{E4}, one or two of R^{E1}, R^{E3}, and R^{E4}, or one or two of R^{E1}, R^{E2}, and R^{E3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

### Exemplary Embodiment 15

The compound of any one of the preceding Exemplary Embodiments, wherein one or two of R^{E1}, R^{E2}, and R^{E4}, one or two of R^{E1}, R^{E3}, and R^{E4}, or one or two of R^{E1}, R^{E2}, and R^{E3} are hydrogen.

### Exemplary Embodiment 16

The compound of any one of the preceding Exemplary Embodiments, wherein each of R^{A2}, R^{A3}, and R^{A4} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

### Exemplary Embodiment 17

The compound of any one of the preceding Exemplary Embodiments, wherein each of R^{A2}, R^{A3}, and R^{A4} is hydrogen.

### Exemplary Embodiment 18

The compound of any one of the preceding Exemplary Embodiments, wherein Ring D is C₃₋₁₂ carbocycle.

### Exemplary Embodiment 19

The compound of any one of the preceding Exemplary Embodiments, wherein Ring D is C₅₋₇ carbocycle.

### Exemplary Embodiment 20

The compound of any one of the preceding Exemplary Embodiments, wherein Ring D is 3- to 12-membered heterocycle.

### Exemplary Embodiment 21

The compound of any one of the preceding Exemplary Embodiments, wherein Ring D is 5- to 7-membered heterocycle.

### Exemplary Embodiment 22

The compound of any one of the preceding Exemplary Embodiments, wherein each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

### Exemplary Embodiment 23

The compound of any one of the preceding Exemplary Embodiments, wherein d is 0.

### Exemplary Embodiment 24

The compound of any one of the preceding Exemplary Embodiments, wherein each R^{C} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

### Exemplary Embodiment 25

The compound of any one of the preceding Exemplary Embodiments, wherein each R^{C} is independently C₁₋₆ alkyl optionally substituted with one or more halogen.

### Exemplary Embodiment 26

The compound of any one of the preceding Exemplary Embodiments, wherein V is of Formula II-3-i

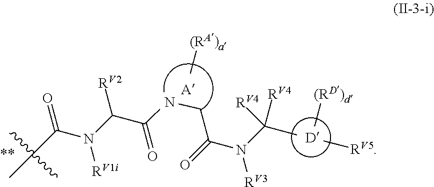

### Exemplary Embodiment 27

The compound of any one of the preceding Exemplary Embodiments, wherein V is of Formula II-3-ii

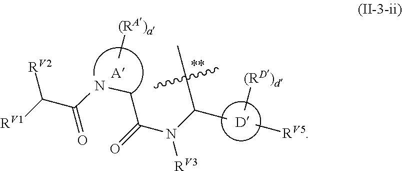

### Exemplary Embodiment 28

The compound of any one of the preceding Exemplary Embodiments, wherein V is of Formula II-3-iii

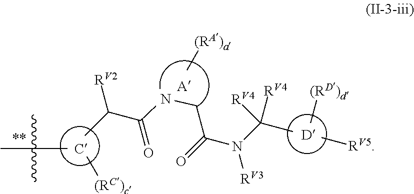

### Exemplary Embodiment 29

The compound of any one of the preceding Exemplary Embodiments, wherein V is of Formula II-3-v

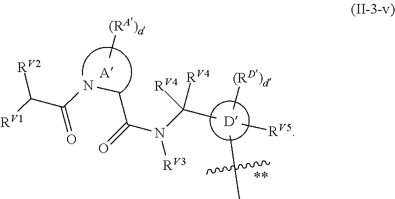

### Exemplary Embodiment 30

The compound of any one of the preceding Exemplary Embodiments, wherein V is of Formula II-3-vi

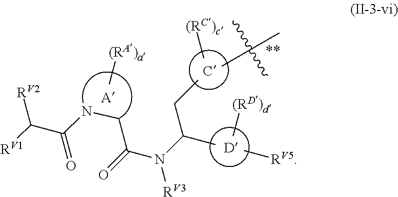

### Exemplary Embodiment 31

The compound of any one of the preceding Exemplary Embodiments, wherein V is of Formula II-3-vii

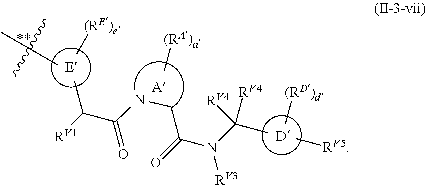

### Exemplary Embodiment 32

The compound of any one of the preceding Exemplary Embodiments, wherein R^{V1} is —N(R^{V1i})C(═O)R^{V1ii} or 5-membered heteroaryl, wherein R^{V1i} is C₃₋₆ carbocyclyl optionally substituted with one or more R^{V1iia}, and the heteroaryl is optionally substituted with one or more R^{u}.

### Exemplary Embodiment 33

The compound of any one of the preceding Exemplary Embodiments, wherein each R^{V1iia} is independently halogen, —CN, C₁₋₆ alkyl, —(C₁₋₆ alkylene)-(3- to 12-membered heterocyclyl), or C₁₋₆ alkylamino, wherein the alkyl, alkylene, or alkylamino is optionally substituted with one or more R^{u}.

### Exemplary Embodiment 34

The compound of any one of the preceding Exemplary Embodiments, wherein R^{V2} is hydrogen, C₁₋₆ alkyl, C₅₋₆ carbocyclyl, or 5- to 6-membered heterocyclyl.

### Exemplary Embodiment 35

The compound of any one of the preceding Exemplary Embodiments, wherein each R^{V4} is independently hydrogen or C₁₋₆ alkyl optionally substituted with one or more R^{V4a}

### Exemplary Embodiment 36

The compound of any one of the preceding Exemplary Embodiments, wherein each R^{V4a} is independently —OH, C₁₋₆ heteroalkyl, C₁₋₆ alkylamino, or 3- to 12-membered heterocyclyl, wherein the heteroalkyl, alkylamino, or heterocyclyl is optionally substituted with one or more R^{u}.

### Exemplary Embodiment 37

The compound of any one of the preceding Exemplary Embodiments, wherein R^{D′} and R^{V4}, together with the atoms to which they are bonded, form C₃₋₁₂ carbocyclyl or 5- to 10-membered heteroaryl, wherein the carbocyclyl or heteroaryl is optionally substituted with one or more R^{u}.

### Exemplary Embodiment 38

The compound of any one of the preceding Exemplary Embodiments, wherein Ring C′ is 5-membered heteroaryl.

### Exemplary Embodiment 39

The compound of any one of the preceding Exemplary Embodiments, wherein each R^{C′} is independently halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl, or heterocyclyl is optionally substituted with one or more R^{u}.

### Exemplary Embodiment 40

The compound of any one of the preceding Exemplary Embodiments, wherein c′ is 0 or 1.

### Exemplary Embodiment 41

The compound of any one of the preceding Exemplary Embodiments, wherein Ring E′ is 5- to 6-membered heterocycle.

### Exemplary Embodiment 42

The compound of any one of the preceding Exemplary Embodiments, wherein each R^{E′} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl, or heterocyclyl is optionally substituted with one or more R^{u}.

### Exemplary Embodiment 43

The compound of any one of the preceding Exemplary Embodiments, wherein e′ is 0.

### Exemplary Embodiment 44

The compound of any one of the preceding Exemplary Embodiments, wherein Ring F′ is 5- to 6-membered heteroaryl.

### Exemplary Embodiment 45

The compound of any one of the preceding Exemplary Embodiments, wherein each R^{F′} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl, or heterocyclyl is optionally substituted with one or more R^{u}.

### Exemplary Embodiment 46

The compound of any one of the preceding Exemplary Embodiments, wherein f′ is 0.

### Exemplary Embodiment 47

The compound of any one of the preceding Exemplary Embodiments, wherein wherein

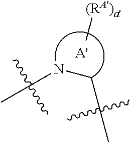

is

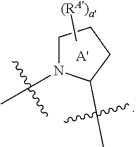

### Exemplary Embodiment 48

The compound of any one of the preceding Exemplary Embodiments, wherein a′ is 1 or 2, and at least one of R^{A′} is —OH.

### Exemplary Embodiment 49

The compound of any one of the preceding Exemplary Embodiments, wherein

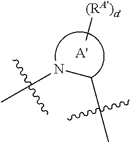

is

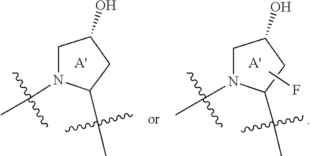

### Exemplary Embodiment 50

The compound of any one of the preceding Exemplary Embodiments, wherein Ring D′ is phenyl.

### Exemplary Embodiment 51

The compound of any one of the preceding Exemplary Embodiments, wherein

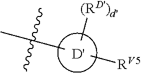

is

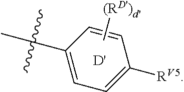

### Exemplary Embodiment 52

The compound of any one of the preceding Exemplary Embodiments, wherein d′ is 0.

### Exemplary Embodiment 53

The compound of any one of the preceding Exemplary Embodiments, wherein R^{V3} is hydrogen.

### Exemplary Embodiment 54

The compound of any one of the preceding Exemplary Embodiments, wherein R^{V5} is halogen, C₂₋₆ alkynyl, C₆₋₁₀ aryl, 5- to 6-membered heteroaryl, or —C(═O)R^{a}, wherein the alkynyl, aryl, or heteroaryl is optionally substituted with one or more R^{u}.

### Exemplary Embodiment 55

The compound of any one of the preceding Exemplary Embodiments, wherein each L′ is independently C₁₋₆ alkylene, C₁₋₆ heteroalkylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O) N(R^{L′})—, —C(═O)O—, —N(R^{L′})—, —O—, or —S(═O)₂—, wherein the alkylene, heteroalkylene, carbocyclylene, heterocyclylene, arylene, or heteroarylene is optionally substituted with one or more R^{u}, and 1 is an integer selected from 1 to 4.

### Exemplary Embodiment 56

The compound of any one of the preceding Exemplary Embodiments, wherein each L′ is independently C₁₋₆ alkylene, C₁₋₆ heteroalkylene, C₂₋₆ alkynylene, C₃₋₁₂ carbocyclylene, 3- to 12-membered heterocyclylene, —C(═O)—, —C(═O) N(R^{L′})—, —C(═O)O—, —N(R^{L′})—, —O—, or —S(═O)₂—, wherein the alkylene, heteroalkylene, carbocyclylene, or heterocyclylene is optionally substituted with one or more R^{u}, and 1 is an integer selected from 1 to 4.

### Exemplary Embodiment 57

The compound of any one of the preceding Exemplary Embodiments, wherein L is absent, C₁₋₁₂ alkylene, C₁₋₆ heteroalkylene, C₃₋₁₂ carbocyclylene, *—C₁₋₁₂ alkylene-O—, *—C₁₋₁₂ alkynylene-O—, *—(C₁₋₁₂ alkylene)-(C₁₋₁₂ alkynylene)-, *—(C₁₋₁₂ alkylene)-O—(C₁₋₁₂ alkynylene)-, *—(C₃₋₁₂ carbocyclylene)-O—, *—(C₁₋₆ alkylene)-(C₃₋₁₂ carbocyclylene)-, *—(C₃₋₁₂ carbocyclylene)-(C₁₋₆ alkylene)-, *—(C₃₋₁₂ carbocyclylene)-(C₁₋₆ alkylene)-O—, *—(C₃₋₁₂ carbocyclylene)-(C₁₋₆ heteroalkylene)-, *—C(═O)—(C₁₋₆ alkylene)-, *—C(═O)—(C₃₋₁₂ carbocyclylene)-, *—C(═O)-(3- to 12-membered heterocyclylene)-, *—(C₁₋₆ alkylene)-(C₃₋₁₂ carbocyclylene)-O—, *-(3- to 12-membered heterocyclylene)-(C₁₋₆ alkylene)-(C₃₋₁₂ carbocyclylene)-, *—(C₁₋₆ alkylene)-(3- to 12-membered heterocyclylene)-, *—(C₁₋₆ alkylene)-(C₃₋₁₂ carbocyclylene)-C(═O) N(R^{L′})—, *-(3- to 12-membered heterocyclylene)-(C₁₋₆ alkylene)-, *—(C₁₋₆ alkylene)-C(═O)-(3- to 12-membered heterocyclylene)-, *—(C₁₋₆ alkylene)-(C₃₋₁₂ carbocyclylene)-C(═O)-(3- to 12-membered heterocyclylene)-, *—(C₃₋₁₂ carbocyclylene)-N(R^{L′})—C(═O)—(C₁₋₆ alkylene)-, *—(C₁₋₆ alkylene)-(C₃₋₁₂ carbocyclylene)-N(R^{L′})C(═O)—(C₁₋₆ alkylene)-, *-(3- to 12-membered heterocyclylene)-C(═O)—, *—(C₁₋₆ alkylene)-(3- to 12-membered heterocyclylene)-C(═O)—, *—(C₁₋₆ alkylene)-(C₃₋₁₂ carbocyclylene)-C(═O) N(R^{L′})—(C₁₋₆ alkylene)-, *—(C₁₋₆ alkylene)-(C₃₋₁₂ carbocyclylene)-C(═O)—(C₁₋₆ alkylene)-, *-(3- to 12-membered heterocyclylene)-C(═O)—(C₁₋₆ alkylene)-, *—(C₃₋₁₂ carbocyclylene)-N(R^{L′})C(═O)—(C₁₋₆ alkylene)-O—, *—(C₁₋₆ alkylene)-N(R^{L′})C(═O)—(C₁₋₆ alkylene)-, *—(C₁₋₆ alkylene)-O—(C₁₋₆ alkylene)-N(R^{L′})C(═O)—, *—(C₁₋₆ alkylene)-(C₃₋₁₂ carbocyclylene)-(C₁₋₆ alkylene)-, *—(C₁₋₆ alkylene)-(C₃₋₁₂ carbocyclylene)-O—, *—(C₁₋₆ alkylene)-(C₆₋₁₀ arylene)-, *—S(═O)₂—(C₃₋₁₂ carbocyclylene)-, or *—(C₁₋₆ alkylene)-(5- to 10-membered heteroarylene)-, wherein the alkylene, heteroalkylene, heterocyclylene, carbocyclylene, arylene, or heteroarylene is optionally substituted with one or more R^{u}, and *denotes attachment to T.

### Exemplary Embodiment 58

A compound selected from the compounds in Tables 1 and 2, or a pharmaceutically acceptable salt thereof.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. F. Eliel, Ed., Univ. of Notre Dame Press, Notre Dame, IN 1972).

The invention additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In certain embodiments, an alkyl group has 1 to 12 carbon atoms (“C₁₋₁₂ alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”, which is also referred to herein as “lower alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkelene” group may be substituted or unsubstituted with one or more substituents as described herein. Exemplary unsubstituted divalent alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted divalent alkylene groups, e.g., substituted with one or more alkyl(methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In certain embodiments, an alkenyl group has 2 to 10 carbon atoms (“C₂₋₁₀ alkenyl”). In certain embodiments, an alkenyl group has 2 to 9 carbon atoms (“C₂₋₉ alkenyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH═CHCH₂—, —CH₂—CH—CH—). Exemplary substituted divalent alkenylene groups, e.g., substituted with one or more alkyl(methyl) groups, include but are not limited to, substituted ethylene (—C(CH₃)—CH—, —CH═C(CH₃)—), substituted propylene (e.g., —C(CH₃)═CHCH₂—, —CH═C(CH₃)CH₂—, —CH—CHCH(CH₃)—, —CH—CHC(CH₃)₂—, —CH(CH₃)—CH—CH—, —C(CH₃)₂—CH═CH—, —CH₂—C(CH₃)—CH—, —CH₂—CH═C(CH₃)—), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In certain embodiments, an alkynyl group has 2 to 10 carbon atoms (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” as used herein, refers to a alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In certain embodiments, a heteroalkyl group is a saturated group having 1 to 9 carbon atoms and 1, 2, 3, or 4 heteroatoms (“heteroC₁₋₉alkyl”). In certain embodiments, a heteroalkyl group is a saturated group having 1 to 8 carbon atoms and 1, 2, 3, or 4 heteroatoms (“heteroC₁₋₈alkyl”). In certain embodiments, a heteroalkyl group is a saturated group having 1 to 7 carbon atoms and 1, 2, 3, or 4 heteroatoms (“heteroC₁₋₇alkyl”). In certain embodiments, a heteroalkyl group is a group having 1 to 6 carbon atoms and 1, 2, or 3 heteroatoms (“heteroC₁₋₆alkyl”). In certain embodiments, a heteroalkyl group is a saturated group having 1 to 5 carbon atoms and 1 or 2 heteroatoms (“heteroC₁₋₅ alkyl”). In certain embodiments, a heteroalkyl group is a saturated group having 1 to 4 carbon atoms and/or 2 heteroatoms (“heteroC₁₋₄alkyl”). In certain embodiments, a heteroalkyl group is a saturated group having 1 to 3 carbon atoms and 1 heteroatom (“heteroC₁₋₃alkyl”). In certain embodiments, a heteroalkyl group is a saturated group having 1 to 2 carbon atoms and 1 heteroatom (“heteroC₁₋₂alkyl”). In certain embodiments, a heteroalkyl group is a saturated group having 1 carbon atom and 1 heteroatom (“heteroC₁ alkyl”). In certain embodiments, a heteroalkyl group is a saturated group having 2 to 6 carbon atoms and 1 or 2 heteroatoms (“heteroC₂₋₆alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀alkyl. In certain embodiments, the heteroalkyl group is a substituted heteroC₁₋₁₀alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀alkenyl”). In certain embodiments, a heteroalkenyl group has 2 to 9 carbon atoms at least one double bond, and 1, 2, 3, or 4 heteroatoms (“heteroC₂₋₉alkenyl”). In certain embodiments, a heteroalkenyl group has 2 to 8 carbon atoms, at least one double bond, and 1, 2, 3, or 4 heteroatoms (“heteroC₂₋₈alkenyl”). In certain embodiments, a heteroalkenyl group has 2 to 7 carbon atoms, at least one double bond, and 1, 2, 3, or 4 heteroatoms (“heteroC₂₋₇alkenyl”). In certain embodiments, a heteroalkenyl group has 2 to 6 carbon atoms, at least one double bond, and 1, 2, or 3 heteroatoms (“heteroC₂₋₆alkenyl”). In certain embodiments, a heteroalkenyl group has 2 to 5 carbon atoms, at least one double bond, and 1 or 2 heteroatoms (“heteroC₂₋₅alkenyl”). In certain embodiments, a heteroalkenyl group has 2 to 4 carbon atoms, at least one double bond, and lor 2 heteroatoms (“heteroC₂₋₄alkenyl”). In certain embodiments, a heteroalkenyl group has 2 to 3 carbon atoms, at least one double bond, and 1 heteroatom (“heteroC₂₋₃alkenyl”). In certain embodiments, a heteroalkenyl group has 2 to 6 carbon atoms, at least one double bond, and 1 or 2 heteroatoms (“heteroC₂₋₆alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀alkenyl. In certain embodiments, the heteroalkenyl group is a substituted heteroC₂₋₁₀alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms are inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“heteroC₂₋₁₀alkynyl”). In certain embodiments, a heteroalkynyl group has 2 to 9 carbon atoms, at least one triple bond, and 1, 2, 3, or 4 heteroatoms (“heteroC₂₋₉alkynyl”). In certain embodiments, a heteroalkynyl group has 2 to 8 carbon atoms, at least one triple bond, and 1, 2, 3, or 4 heteroatoms (“heteroC₂₋₈alkynyl”). In certain embodiments, a heteroalkynyl group has 2 to 7 carbon atoms, at least one triple bond, and 1, 2, 3, or 4 heteroatoms (“heteroC₂₋₇alkynyl”). In certain embodiments, a heteroalkynyl group has 2 to 6 carbon atoms, at least one triple bond, and 1, 2, or 3 heteroatoms (“heteroC₂₋₆alkynyl”). In certain embodiments, a heteroalkynyl group has 2 to 5 carbon atoms, at least one triple bond, and 1 or 2 heteroatoms (“heteroC₂₋₅alkynyl”). In certain embodiments, a heteroalkynyl group has 2 to 4 carbon atoms, at least one triple bond, and lor 2 heteroatoms (“heteroC₂₋₄alkynyl”). In certain embodiments, a heteroalkynyl group has 2 to 3 carbon atoms, at least one triple bond, and 1 heteroatom (“heteroC₂-3 alkynyl”). In certain embodiments, a heteroalkynyl group has 2 to 6 carbon atoms, at least one triple bond, and 1 or 2 heteroatoms (“heteroC₂₋₆alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀alkynyl. In certain embodiments, the heteroalkynyl group is a substituted heteroC₂₋₁₀alkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene,” “heteroalkenylene,” or “heteroalkynylene,” group, it is understood that the range or number refers to the range or number of carbons in the linear divalent chain. “Heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl).

Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Arylene” as used herein, refers to an aryl group wherein two hydrogens are removed to provide a divalent radical. When a range or number of carbons is provided for a particular “arylene” group, it is understood that the range or number refers to the range or number of carbons in the aryl group. An “arylene” group may be substituted or unsubstituted with one or more substituents as described herein.

“Heteroaryl” refers to a radical of a 5- to 14-membered monocyclic or polycyclic 4n+2 aromatic ring system (e.g., having 6, 10, or 14 π electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is either on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In certain embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In certain embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In certain embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In certain embodiments, the 5- to 6-membered heteroaryl has 1-3 ring heteroatoms independently selected from nitrogen, oxygen, and sulfur. In certain embodiments, the 5- to 6-membered heteroaryl has 1-2 ring heteroatoms independently selected from nitrogen, oxygen, and sulfur. In certain embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

Exemplary 5-membered heteroaryl containing one heteroatom include, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Heteroarylene” as used herein, refers to a heteroaryl group wherein two hydrogens are removed to provide a divalent radical. When a range or number of ring members is provided for a particular “heteroarylene” group, it is understood that the range or number refers to the number of ring members in the heteroaryl group. A “heteroarylene” group may be substituted or unsubstituted with one or more substituents as described herein.

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 6 ring carbon atoms (“C₃₋₆ carbocyclyl”). In certain embodiments, a carbocyclyl group has 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, a carbocyclyl group has 5 or 6 ring carbon atoms (“C₅₋₆ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₅), bicyclo[2.2.1]heptanyl (C₇), bicyclo[2.2.2]octanyl (C₈), and the like. Exemplary C₃₋₁₀ carbocyclyl include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl(C₁₀), and the like.

In certain embodiments, “carbocyclyl” is a monocyclic, saturated carbocyclyl group having from 3 to 12 ring carbon atoms (“C₃₋₁₂ carbocyclyl”). In certain embodiments, “carbocyclyl” is a monocyclic, saturated carbocyclyl group having from 3 to 10 ring carbon atoms (“C₃₋₁₀ carbocyclyl”). In certain embodiments, “carbocyclyl” is a monocyclic, saturated carbocyclyl group having from 3 to 8 ring carbon atoms (“C₃₋₈ carbocyclyl”). In certain embodiments, “carbocyclyl” is a monocyclic, saturated carbocyclyl group having from 3 to 6 ring carbon atoms (“C₃₋₆ carbocyclyl”). In certain embodiments, “carbocyclyl” is a monocyclic, saturated carbocyclyl group having from 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl (C₅) and cyclohexyl (C₅). Examples of C₃₋₆ carbocyclyl include the aforementioned C₅₋₆ carbocyclyl groups as well as cyclopropyl (C₃) and cyclobutyl (C₄). Examples of C₃₋₈ carbocyclyl include the aforementioned C₃₋₆ carbocyclyl groups as well as cycloheptyl (C₇) and cyclooctyl (C₈). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is substituted C₃₋₁₂ carbocyclyl.

As the foregoing examples illustrate, in certain embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is a substituted C₃₋₁₂ carbocyclyl.

“Fused carbocyclyl” or “fused carbocycle” refers to ring systems wherein the carbocyclyl group, as defined above, is fused with, i.e., share two common atoms (as such, share one common bond), one or more carbocyclyl groups, as defined above, wherein the point of attachment is on any of the fused rings. In such instances, the number of carbons designates the total number of carbons in the fused ring system. When substitution is indicated, unless otherwise specified, substitution can occur on any of the fused rings.

“Spiro carbocyclyl” or or “spiro carbocycle” refers to ring systems wherein the carbocyclyl group, as defined above, form spiro structure with, i.e., share one common atom with, one or more carbocyclyl groups, as defined above, wherein the point of attachment is on the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on the carbocyclyl rings in which the spiro structure is embedded.

“Bridged carbocyclyl” or or “bridged carbocycle” refers to ring systems wherein the carbocyclyl group, as defined above, form bridged structure with, i.e., share more than two atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the carbocyclyl rings in which the bridged structure is embedded. When substitution is indicated, unless otherwise specified, substitution can occur on any of the carbocyclyl rings in which the bridged structure is embedded.

“Carbocyclylene” as used herein, refers to a carbocyclyl group wherein two hydrogens are removed to provide a divalent radical. The divalent radical may be present on different atoms or the same atom of the carbocycle group. When a range or number of carbons is provided for a particular “carbocyclyl” group, it is understood that the range or number refers to the range or number of carbons in the carbocyclyl group. A “carbocyclyl” group may be substituted or unsubstituted with one or more substituents as described herein.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5 membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

As the foregoing examples illustrate, in certain embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 12-membered heterocyclyl. In certain embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share two common atoms (as such, share one common bond) with, one or more heterocyclyl or carbocyclyl groups, as defined above, wherein the point of attachment is on any of the fused rings. In such instances, the number of ring members designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than two atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the heterocyclyl or carbocyclyl rings in which the bridged structure is embedded.

“Heterocyclylene” as used herein, refers to a heterocyclyl group wherein two hydrogens are removed to provide a divalent radical. The divalent radical may be present on different atoms or the same atom of the heterocyclylene group. When a range or number of ring members is provided for a particular “heterocyclylene” group, it is understood that the range or number refers to the number of ring members in the heterocyclylene group. A “heterocyclylene” group may be substituted or unsubstituted with one or more substituents as described herein.

“Alkoxy” as used herein, refers to the group-OR, wherein R is alkyl as defined herein. C₁₋₆ alkoxy refers to the group-OR, wherein each R is C₁₋₆ alkyl, as defined herein. Exemplary C₁₋₆ alkyl is set forth above.

“Alkylamino” as used herein, refers to the group —NHR or —NR², wherein each R is independently alkyl, as defined herein. C₁₋₆ alkylamino refers to the group —NHR or —NR², wherein each R is independently C₁₋₆ alkyl, as defined herein. Exemplary C₁₋₆ alkyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Halo” or “halogen” refers to fluoro (F), chloro (Cl), bromo (Br), and iodo (I). In certain embodiments, the halo group is either fluoro or chloro.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction. Common functional groups that need to be protected include but not limited to hydroxyl, amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), β-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g., t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g, benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g., p-methylbenzyl (Meb), t-butyl, acetamidomethyl (Acm), and triphenylmethyl (Trityl)).

Common types of carboxylic acid-protecting groups include but not limited to esters (e.g., methyl ester, triphenylmethyl (Trityl), t-butyl ester, benzyl ester (Bn), S-t-butyl ester, silyl esters, and orthoesters) and oxazoline.

These and other exemplary substituents are described in more detail in the Detailed Description, Examples, and claims. The invention is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

An “effective amount” means the amount of a compound that, when administered to a subject for treating or preventing a disease, is sufficient to affect such treatment or prevention. The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

“Preventing”, “prevention” or “prophylactic treatment” refers to a reduction in risk of acquiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

The term “about” when referring to a number or a numerical range means that the number or numerical range referred to is an approximation within experimental variability or within statistical experimental error, and thus the number or numerical range, in some instances, will vary between 1% and 15% of the stated number or numerical range. In certain embodiments, the number or numerical range vary by 1%, 2%, 3%, 4%, 5%, 6%, 7%, 8%, 9%, 10%, 11%, 12%, 13%, 14%, or 15% of the stated number or numerical range. In certain embodiments, the number or numerical range vary by 1%, 2%, 3%, 4%, or 5% of the stated number or numerical range. In certain embodiments, the number or numerical range vary by 1%, 2%, or 3% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### Examples

In order that the invention described herein may be more fully understood, the following examples are set forth. The examples described in this application are offered to illustrate the compounds, pharmaceutical compositions, and methods provided herein and are not to be construed in any way as limiting their scope.

### I. Synthesis and Characterization

Compound A². (2S,4R)—N—((S)-6-(4-(4′-chloro-5′-oxo-5′H-spiro[cyclopentane-1,7′-indolo[1,2-a]quinazolin]-9′-yl) piperidin-1-yl)-1-(4-(4-methylthiazol-5-yl)phenyl) hexyl)-1-((S)-2-(1-fluorocyclopropane-1-carboxamido)-3,3-dimethylbutanoyl)-4-hydroxypyrrolidine-2-carboxamide

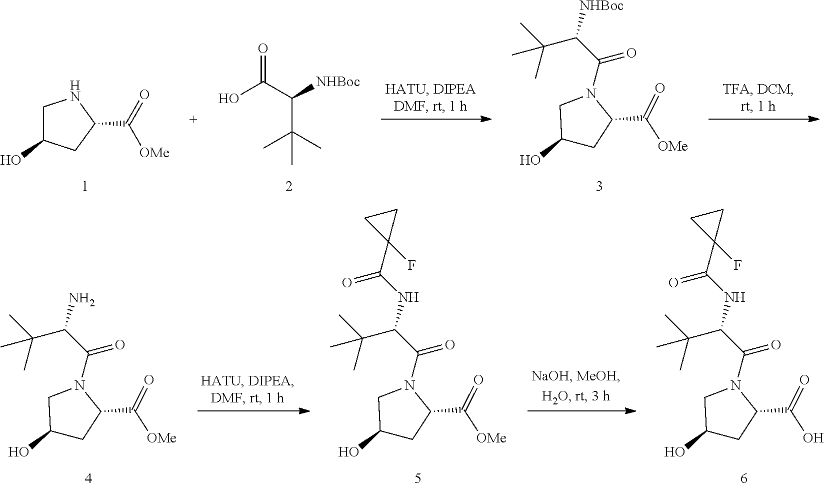 [SMILES: COC(=O)[C@@H]1C[C@@H](O)CN1 ; CC(C)(C)OC(=O)N[C@H](C(=O)O)C(C)(C)C ; COC(=O)[C@@H]1C[C@@H](O)CN1C(=O)[C@@H](NC(=O)OC(C)(C)C)C(C)(C)C ; COC(=O)[C@@H]1C[C@@H](O)CN1C(=O)[C@@H](N)C(C)(C)C ; COC(=O)[C@@H]1C[C@@H](O)CN1C(=O)[C@@H](NC(=O)C1(F)CC1)C(C)(C)C ; CC(C)(C)[C@H](NC(=O)C1(F)CC1)C(=O)N1C[C@H](O)C[C@H]1C(=O)O]

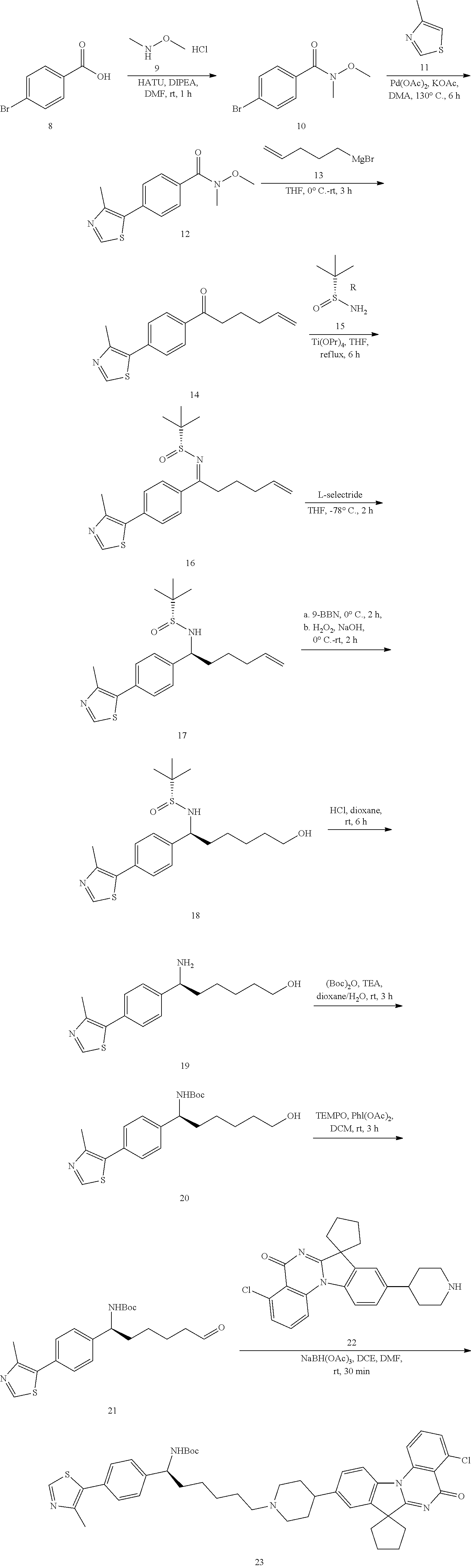

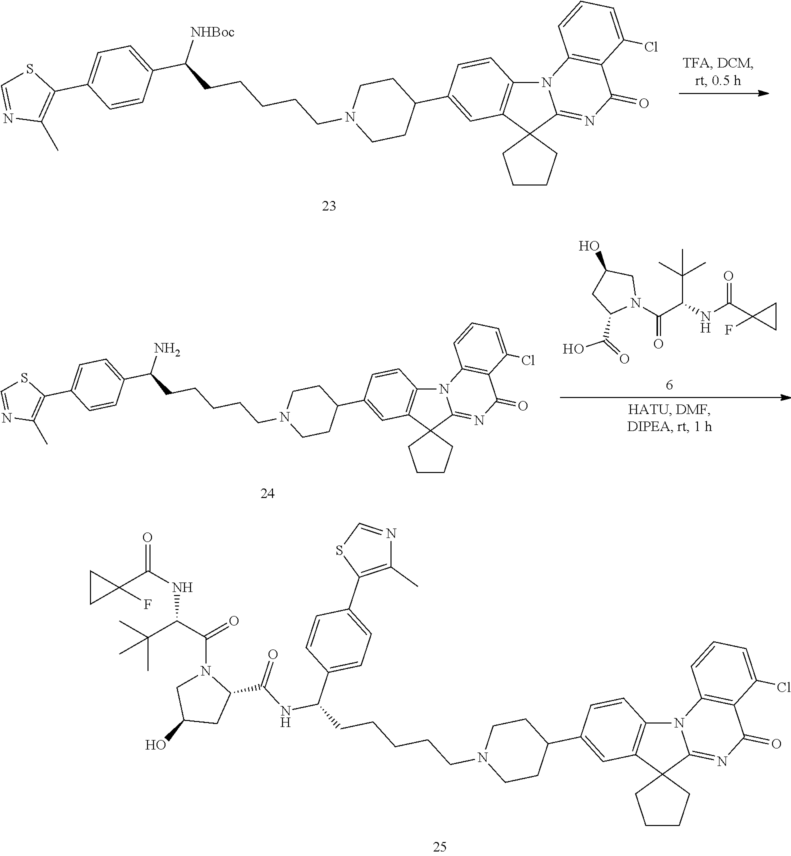 [SMILES: Cc1ncsc1-c1ccc([C@H](CCCCCN2CCC(c3ccc4c(c3)C3(CCCC3)c3nc(=O)c5c(Cl)cccc5n3-4)CC2)NC(=O)OC(C)(C)C)cc1 ; Cc1ncsc1-c1ccc([C@@H](N)CCCCCN2CCC(c3ccc4c(c3)C3(CCCC3)c3nc(=O)c5c(Cl)cccc5n3-4)CC2)cc1 ; CC(C)(C)[C@H](NC(=O)C1(F)CC1)C(=O)N1C[C@H](O)C[C@H]1C(=O)O ; Cc1ncsc1-c1ccc([C@H](CCCCCN2CCC(c3ccc4c(c3)C3(CCCC3)c3nc(=O)c5c(Cl)cccc5n3-4)CC2)NC(=O)[C@@H]2C[C@@H](O)CN2C(=O)[C@@H](NC(=O)C2(F)CC2)C(C)(C)C)cc1]

To a solution of 1 (3.00 g, 20.7 mmol, 1 equiv.) and 2 (4.78 g, 20.7 mmol, 1 equiv.) in DMF (50 mL) was added HATU (9.43 g, 24.8 mmol, 1.2 equiv.) and DIPEA (5.34 g, 41.4 mmol, 2 equiv.). The mixture was stirred for 1 h. Water (80 mL) was added to quench the reaction and product was extracted with ethyl acetate twice (60 mL×2). Combined organic phases were washed by HCl (1 N, 50 mL) once, saturated NaHCO₃ aqueous solution once, and brine once, dried by anhydrous Na₂SO₄. After removing solvent, the received crude product used in the next step directly. ESI MS m/z: 359.20 [M+H]⁺.

Preparation of 4. To a solution of crude 3 in DCM (30 mL) was added TFA (20 mL). The mixture was stirred for 1 h. After removing solvent, A crude product was received as a TFA salt (6.7 g, 91% for two steps) and used in the next step directly. ESI MS m/z: 259.15 [M+H]⁺.

Preparation of 5. To a solution of crude product 4 (6.70 g, 18.86 mmol, 1 equiv.) and 1-fluorocyclopropane-1-carboxylic acid (2.35 g, 22.63 mmol, 1.2 equiv.) in DMF (50 mL) was added HATU (10.75 g, 28.28 mmol, 1.5 equiv.) and DIPEA (7.31 g, 56.57 mmol, 3 equiv.). The mixture was stirred for 1 h. Water (80 mL) was added to quench the reaction and product was extracted with ethyl acetate twice (60 mL×2). Combined organic phases were washed by HCl (1 N, 50 mL) once, saturated NaHCO₃ aqueous solution once, and brine once, dried by anhydrous Na₂SO₄. After removing solvent, the received crude product was purified by silica gel column chromatography (ethyl acetate/hexane=2/1) to get product 5 (5.1 g, 79%). ESI MS m/z: 345.17 [M+H]⁺. 1H NMR (400 MHz, CDCl3) δ 7.17 (dd, J=9.5, 3.7 Hz, 1H), 4.67 (dd, J=9.5, 7.8 Hz, 1H), 4.60 (dd, J=9.3, 1.3 Hz, 1H), 4.55-4.48 (m, 1H), 4.02 (dt, J=11.4, 1.7 Hz, 1H), 3.75 (m, 4H), 2.37 (ddt, J=13.4, 7.9, 1.9 Hz, 1H), 2.03-1.96 (m, 1H), 1.40-1.21 (m, 4H), 1.08 (s, 9H).

Preparation of 6. To a solution of crude product 5 (5.00 g, 14.52 mmol, 1 equiv.) in MeOH/H₂O (50 mL/5 mL) was added NaOH (1.16 g, 28.28 mmol, 2.0 equiv.) The mixture was stirred for 3 h. MeOH was removed under rotatory evaporator. The aqueous phase was added TFA until the pH reach 4. The crude product aqueous solution was purified by C-18 reversal column (elution solvents: CH₃CN/H₂O from 10% to 100%, 0.1% TFA) to get pure product (4.4 g, 92%). ESI MS m/z: 331.15 [M+H]⁺. 1H NMR (400 MHz, DMSO) δ 12.81 (s, 1H), 7.26 (dd, J=9.3, 3.0 Hz, 1H), 4.60 (dd, J=9.4, 1.3 Hz, 1H), 4.44-4.27 (m, 2H), 3.66 (dd, J=10.8, 3.9 Hz, 1H), 3.63-3.56 (m, 1H), 2.14 (ddt, J=11.8, 7.9, 1.8 Hz, 1H), 1.91 (ddd, J=13.3, 9.3, 4.5 Hz, 1H), 1.42-1.37 (m, 1H), 1.37-1.31 (m, 1H), 1.26-1.14 (m, 2H), 0.99 (s, 9H).

Preparation of 10. To a solution of 8 (10.00 g, 49.75 mmol, 1 equiv.) and 9 (5.82 g, 59.70 mmol, 1.2 equiv.) in DMF (80 mL) was added HATU (28.37 g, 74.62 mmol, 1.5 equiv.) and DIPEA (19.29 g, 149.24 mmol, 3 equiv). The mixture was stirred for 1 h. Water (150 mL) was added to quench the reaction and product was extracted with ethyl acetate twice (100 mL×2). Combined organic phases were washed by HCl (1 N, 80 mL) once, saturated NaHCO₃ aqueous solution once, and brine once, dried by anhydrous Na₂SO₄. After removing solvent, the received crude product (11.5 g, 95%) used in the next step directly. ESI MS m/z: 244.00 [M+H]⁺.

Preparation of 12. To a solution of 10 (9.50 g, 38.92 mmol, 1 equiv.) and 11 (7.72 g, 77.84 mmol, 2.0 equiv.) in DMF (80 mL) was added Pd (OAc) 2 (873 mg, 3.89 mmol, 0.1 equiv.) and KOAc (7.64 g, 77.84 mmol, 2 equiv.). The mixture was stirred for 6 h at 120° C. Water (150 mL) was added to quench the reaction and product was extracted with ethyl acetate twice (100 mL×2). Combined organic phases were washed by brine twice, dried by anhydrous Na₂SO₄. After removing solvent, the received crude product was purified by silica gel column chromatography (ethyl acetate/hexane=1/1) to get pure product (9.5 g, 93%). ESI MS m/z: 263.08 [M+H]⁺.

Preparation of 14. To a solution of 12 (9.50 g, 36.21 mmol, 1 equiv.) in dry THF (180 mL) at 0° C. was added 13 (40 ml, 40 mmol, 1.1 equiv.). The mixture was warmed to room temperature and stirred for 3 h. A saturated NH₄Cl (100 mL) was added to quench the reaction and product was extracted with ethyl acetate twice (100 mL×2). Combined organic phases were dried by anhydrous Na₂SO₄. After removing solvent, the received crude product was purified by silica gel column chromatography (ethyl acetate/hexane=1/1) to get pure product (4.6 g, 47%). ESI MS m/z: 272.10 [M+H]⁺. 1H NMR (400 MHz, CDCl3) δ 8.77 (s, 1H), 8.10-8.00 (m, 2H), 7.61-7.53 (m, 2H), 5.86 (ddt, J=17.0, 10.2, 6.7 Hz, 1H), 5.09 (dq, J=17.1, 1.6 Hz, 1H), 5.04 (ddt, J=10.2, 2.2, 1.2 Hz, 1H), 3.03 (t, J=7.3 Hz, 2H), 2.61 (s, 3H), 2.26-2.15 (m, 2H), 1.90 (p, J=7.3 Hz, 2H).

Preparation of 16. To a solution of 14 (4.50 g, 16.58 mmol, 1 equiv.) in dry THF (100 mL) was added 15 (2.41 g, 19.90 mmol, 1.2 equiv.) and Ti(i-OPr)₄ (9.43 g, 33.16 mmol, 2.0 equiv.). The mixture was refluxed for 16 h. After cooling to room temperature, water (10 mL) was added to reaction mixture. The formed solid was filtered and the filtrate was concentrated under rotatory evaporator. The residue was redissolved into a mixture of ethyl acetate and water, the aqueous layer was separated and organic phases were dried by anhydrous Na₂SO₄. After removing solvent, the received crude product was purified by silica gel column chromatography (ethyl acetate/hexane=3/2) to get pure product (4.6 g, 74%). ESI MS m/z: 375.15 [M+H]⁺.

Preparation of 17. To a solution of 16 (4.50 g, 12.0 mmol, 1 equiv.) in dry THF (100 mL) was added L-selectride (18.0 mL, 18.00 mmol, 1.5 equiv.) at −78° C. The mixture was stirred for 3 h at temperature. A saturated NH₄Cl aqueous solution was (50 mL) was added to reaction mixture. The product was extracted with ethyl acetate twice (100 mL×2). Combined organic phases were dried by anhydrous Na₂SO₄. After removing solvent, the received crude product was purified by C-18 reversal column (elution solvents: CH₃CN/H₂O from 10% to 100%, 0.1% TFA) to get pure product (3.5 g, 77%). ESI MS m/z: 377.15 [M+H]⁺.

Preparation of 18. To a solution of 17 (3.50 g, 9.29 mmol, 1 equiv.) in dry THF (60 mL) was added 9-BBN (11.5 mL, 11.50 mmol, 1.2 equiv.) at 0° C. The mixture was stirred for 2 h at 0° C. H₂O (15 mL) was added to quench the reaction mixture, then H₂O₂ (5 mL, 30%) and NaOH (1.86 g, 46.47 mmol, 5 equiv.) were added. Let the mixture warm to room temperature and stir for 2 h. The product was extracted with ethyl acetate twice (60 mL×2). Combined organic phases were washed by brine once, dried by anhydrous Na₂SO₄. After removing solvent, the received crude product was purified by C-18 reversal column (elution solvents: CH₃CN/H₂O from 10% to 100%, 0.1% TFA) to get pure product (3.1 g, 85%). ESI MS m/z: 395.15 [M+H]⁺.

Preparation of 19. To a solution of 18 (3.00 g, 7.60 mmol, 1 equiv.) in dry dioxane (10 mL) was added HCl dioxane solution (10 mL, 4N) at 0° C. The mixture was stirred for 2 h at rt. After removing dioxane, the received crude product was used in the next step directly. ESI MS m/z: 291.15 [M+H]⁺.

Preparation of 20. To a solution of crude 19 obtained from the last step in dioxane (30 mL) was added H₂O (10 mL), TEA (2.30 g, 22.8 mmol, 3 equiv.), and (Boc)₂O (1.99 g, 9.12 mmol, 1.2 equiv.). The mixture was stirred for 3 h at rt. After removing dioxane, the product was extracted with ethyl acetate twice (30 mL×2). Combined organic phases were washed by brine once, dried by anhydrous Na₂SO₄. After removing solvent, the received crude product was purified by silica gel column chromatography (ethyl acetate/hexane=1/2) to get pure product (2.1 g, 71% for two steps). ESI MS m/z: 391.20 [M+H]⁺.

Preparation of 21. To a solution of 20 (2.00 g, 5.12 mmol, 1 equiv.) in DCM (50 mL) was added PhI (OAc) 2 (2.45 g, 7.68 mmol, 1.5 equiv.), TEMPO (160 0 g, 1.02 mmol, 0.2 equiv.). The mixture was stirred for 3 h under nitrogen protection. After reaction, a saturated Na₂S₂O₃ was added and stirred for 15 min. the DCM layer was collected, washed by brine once. After removing solvent, the received crude product was purified by C-18 reversal column (elution solvents: CH₃CN/H₂O from 10% to 100%, 0.1% TFA) to get pure product (1.68 g, 84%). ESI MS m/z: 389.19 [M+H]⁺.

Preparation of 23. To a solution of 21 (200 mg, 0.514 mmol, 1 equiv.) in DCE/DMF (5/5 mL) was added 22 (156 mg, 0.386 mmol, 0.75 equiv.). The mixture was stirred for 15 min. then NaBH (OAc) 3 (330 mg, 1.54 mmol, 3 equiv.) was added. let the reaction mixture stirred for additional 15 min. After reaction, TFA (0.5 mL) was added to quench the reaction. The DCE was removed under rotatory evaporator. The residue was purified by C-18 reversal column (elution solvents: CH₃CN/H₂O from 10% to 100%, 0.1% TFA) to get pure product (260 mg, 65%). ESI MS m/z: 778.35 [M+H]⁺.

Preparation of 24. To a solution of 23 (25 mg, 0.032 mmol, 1 equiv.) in DCM (3 mL) was added TFA (3 mL). The mixture was stirred for 30 min. After reaction, the DCM and TFA were removed under rotatory evaporator. The residue was used in the next step directly. ESI MS m/z: 678.30 [M+H]⁺.

Preparation of 25. To a solution of crude 24, and 6 (10.6 mg, 0.321 mmol, 1 equiv.) in DMF (5 mL) was added HATU (18.32 mg, 0.48 mmol, 1.5 equiv.) and DIPEA (12.5 mg, 0.41 mmol, 2 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (16.0 mg, 50% for two steps). ESI MS m/z: 990.44 [M+H]⁺.

Compound A³. (2S,4R)—N—((S)-6-(4-(4′-chloro-5′-oxo-5′H-spiro[cyclohexane-1,7′-indolo[1,2-a]quinazolin]-9′-yl) piperidin-1-yl)-1-(4-(4-methylthiazol-5-yl)phenyl) hexyl)-1-((S)-2-(1-fluorocyclopropane-1-carboxamido)-3,3-dimethylbutanoyl)-4-hydroxypyrrolidine-2-carboxamide

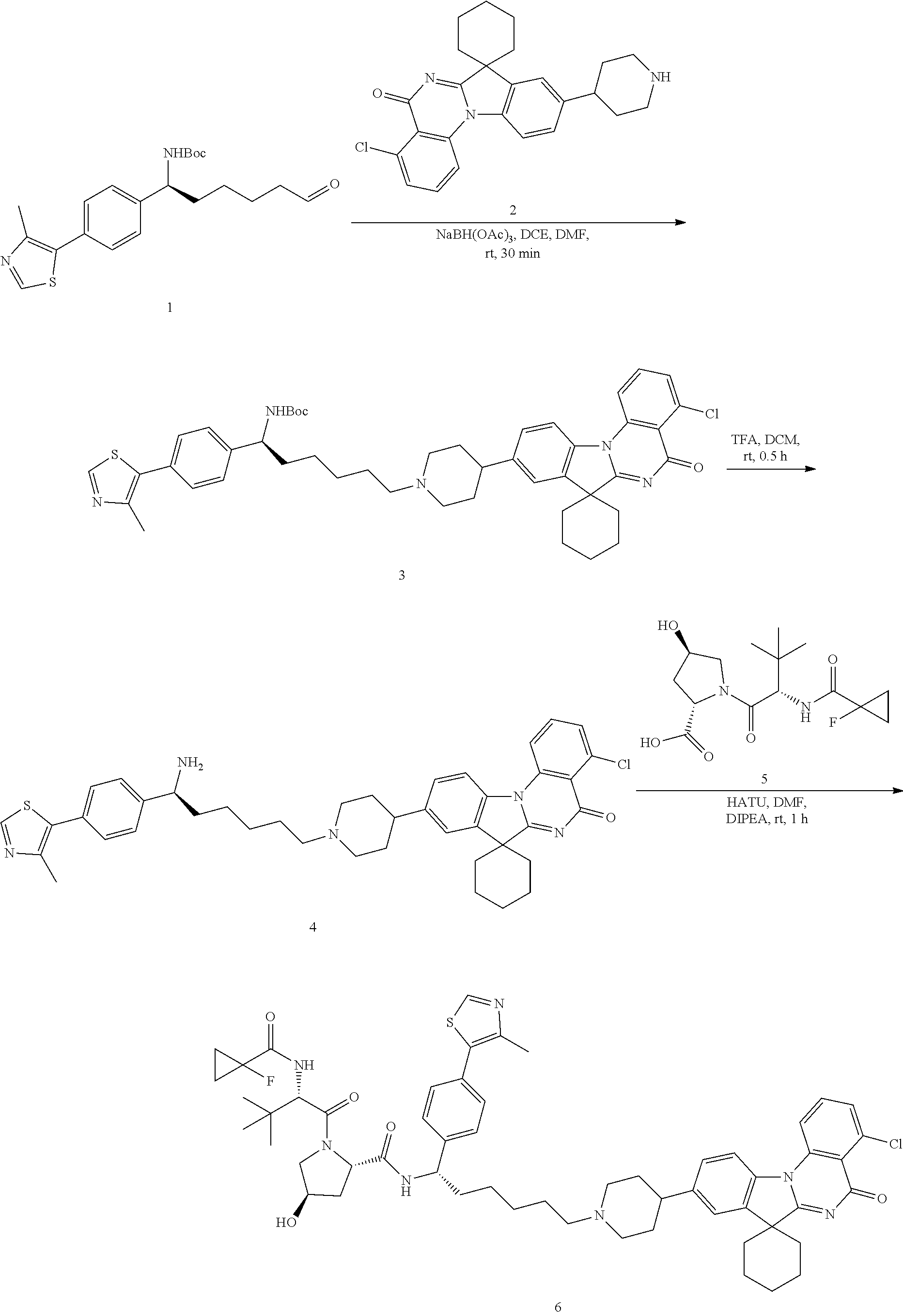

Preparation of 3. To a solution of 1 (100 mg, 0.25 mmol, 1 equiv.) in DCE/DMF (5/5 mL) was added 2 (81 mg, 0.193 mmol, 0.75 equiv.). The mixture was stirred for 15 min. then NaBH (OAc) 3 (163 mg, 0.77 mmol, 3 equiv.) was added. let the reaction mixture stirred for additional 15 min. After reaction, TFA (0.5 mL) was added to quench the reaction. The DCE was removed under rotatory evaporator. The residue was purified by C-18 reversal column (elution solvents: CH₃CN/H₂O from 10% to 100%, 0.1% TFA) to get pure product (120 mg, 59%). ESI MS m/z: 792.36 [M+H]⁺.

Preparation of 4. To a solution of 3 (50 mg, 0.063 mmol, 1 equiv.) in DCM (3 mL) was added TFA (3 mL). The mixture was stirred for 30 min. After reaction, the DCM and TFA were removed under rotatory evaporator. The residue was used in the next step directly. ESI MS m/z: 692.31 [M+H]⁺.

Preparation of 6. To a solution of crude 4, and 5 (20.8 mg, 0.063 mmol, 1 equiv.) in DMF (5 mL) was added HATU (36.0 mg, 0.094 mmol, 1.5 equiv.) and DIPEA (24.46 mg, 0.19 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (36.0 mg, 57% for two steps). ESI MS m/z: 1004.45 [M+H]⁺.

Compound A⁴. (2S,4R)—N—((S)-6-(4-(4′-bromo-5′-oxo-5′H-spiro[cyclohexane-1,7′-indolo[1,2-a]quinazolin]-9′-yl) piperidin-1-yl)-1-(4-(4-methylthiazol-5-yl)phenyl) hexyl)-1-((S)-2-(1-fluorocyclopropane-1-carboxamido)-3,3-dimethylbutanoyl)-4-hydroxypyrrolidine-2-carboxamide

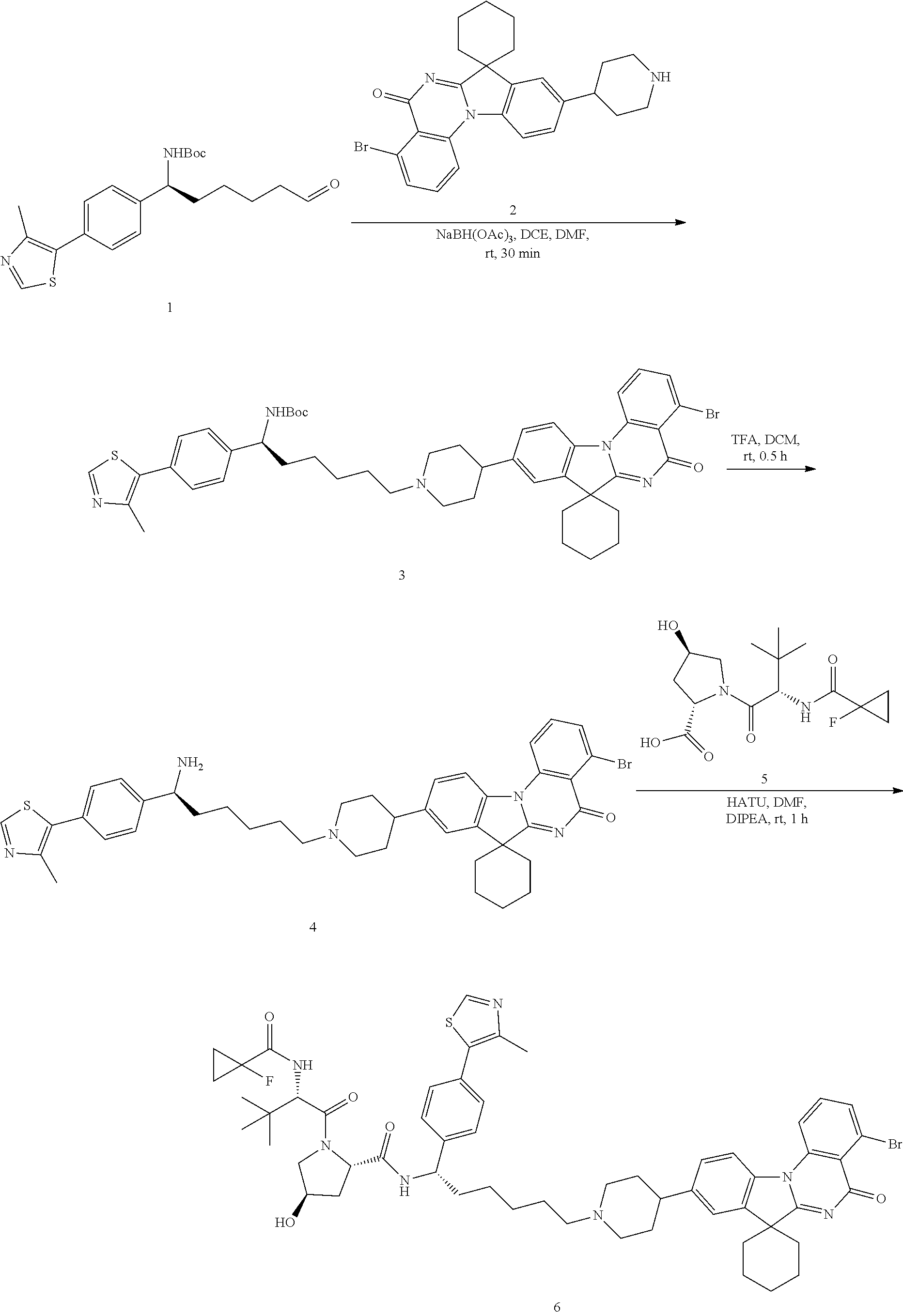

Preparation of 3. To a solution of 1 (50 mg, 0.13 mmol, 1 equiv.) in DCE/DMF (5/5 mL) was added 2 (44.8 mg, 0.096 mmol, 0.75 equiv.). The mixture was stirred for 15 min. then NaBH (OAc) 3 (81.8 mg, 0.39 mmol, 3 equiv.) was added. let the reaction mixture stirred for additional 15 min. After reaction, TFA (0.5 mL) was added to quench the reaction. The DCE was removed under rotatory evaporator. The residue was purified by C-18 reversal column (elution solvents: CH₃CN/H₂O from 10% to 100%, 0.1% TFA) to get pure product (60 mg, 56%). ESI MS m/z: 838.31 [M+H]⁺.

Preparation of 4. To a solution of 3 (50 mg, 0.063 mmol, 1 equiv.) in DCM (3 mL) was added TFA (3 mL). The mixture was stirred for 30 min. After reaction, the DCM and TFA were removed under rotatory evaporator. The residue was used in the next step directly. ESI MS m/z: 738.26 [M+H]⁺.

Preparation of 6. To a solution of crude 4, and 5 (23.6 mg, 0.072 mmol, 1 equiv.) in DMF (5 mL) was added HATU (34.0 mg, 0.090 mmol, 1.5 equiv.) and DIPEA (23.2 mg, 0.18 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (28.0 mg, 45% for two steps). ESI MS m/z: 526.00 [M/2+H]⁺. 1H NMR (400 MHz, MeOD) δ 8.93 (d, J=1.6 Hz, 1H), 8.45 (dd, J=8.7, 1.0 Hz, 1H), 8.14-8.07 (m, 1H), 7.91 (dd, J=7.9, 0.9 Hz, 1H), 7.83-7.67 (m, 2H), 7.49-7.44 (m, 4H), 4.90 (m, 1H), 4.76 (t, J=2.2 Hz, 2H), 4.63 (dd, J=9.4, 7.5 Hz, 1H), 4.48 (s, 1H), 3.88 (d, J=11.4 Hz, 1H), 3.82-3.69 (m, 3H), 3.19 (dt, J=17.0, 10.6 Hz, 4H), 2.51 (d, J=1.8 Hz, 3H), 2.30-1.48 (m, 25H), 1.46-1.25 (m, 4H), 1.10 (d, J=3.4 Hz, 9H).

Compound A⁵. (2S,4R)-1-((S)-2-(2-(4-(4′-chloro-5′-oxo-5′H-spiro[cyclohexane-1,7′-indolo[1,2-a]quinazolin]-9′-yl) piperidin-1-yl) acetamido)-3,3-dimethylbutanoyl)-4-hydroxy-N—((S)-1-(4-(4-methylthiazol-5-yl)phenyl)ethyl) pyrrolidine-2-carboxamide

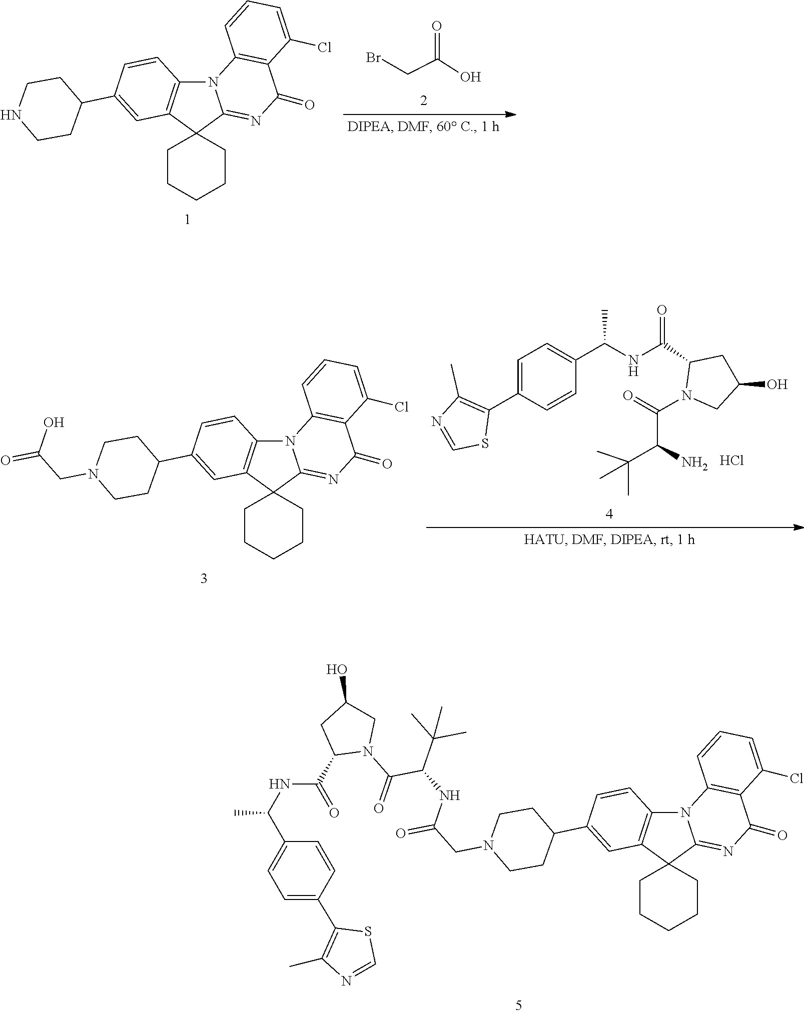

Preparation of 3. To a solution of 1 (50 mg, 0.12 mmol, 1.0 equiv.) in DMF (2 mL) was added 2 (16.5 mg, 0.12 mmol, 0.1 equiv.) and DIPEA (46.2 mg, 0.36 mmol, 3.0 equiv.). The mixture was stirred for 1 h at 60° C. The residue was purified by C-18 reversal column (elution solvents: CH₃CN/H₂O from 10% to 100%, 0.1% TFA) to get pure product (36 mg, 63%). ESI MS m/z: 478.18 [M+H]⁺.

Preparation of 5. To a solution of 3 (20 mg, 0.041 mmol, 1.0 equiv.), and 4 (13.8 mg, 0.041 mmol, 1.0 equiv.) in DMF (2 mL) was added HATU (24.0 mg, 0.063 mmol, 1.5 equiv.) and DIPEA (16.2 mg, 0.13 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (23.0 mg, 61%). ESI MS m/z: 904.40 [M+H]⁺.

Compound A⁶. (2S,4R)-1-((S)-2-(3-(4-(4′-chloro-5′-oxo-5′H-spiro[cyclohexane-1,7′-indolo[1,2-a]quinazolin]-9′-yl) piperidin-1-yl) propanamido)-3,3-dimethylbutanoyl)-4-hydroxy-N—((S)-1-(4-(4-methylthiazol-5-yl)phenyl)ethyl) pyrrolidine-2-carboxamide

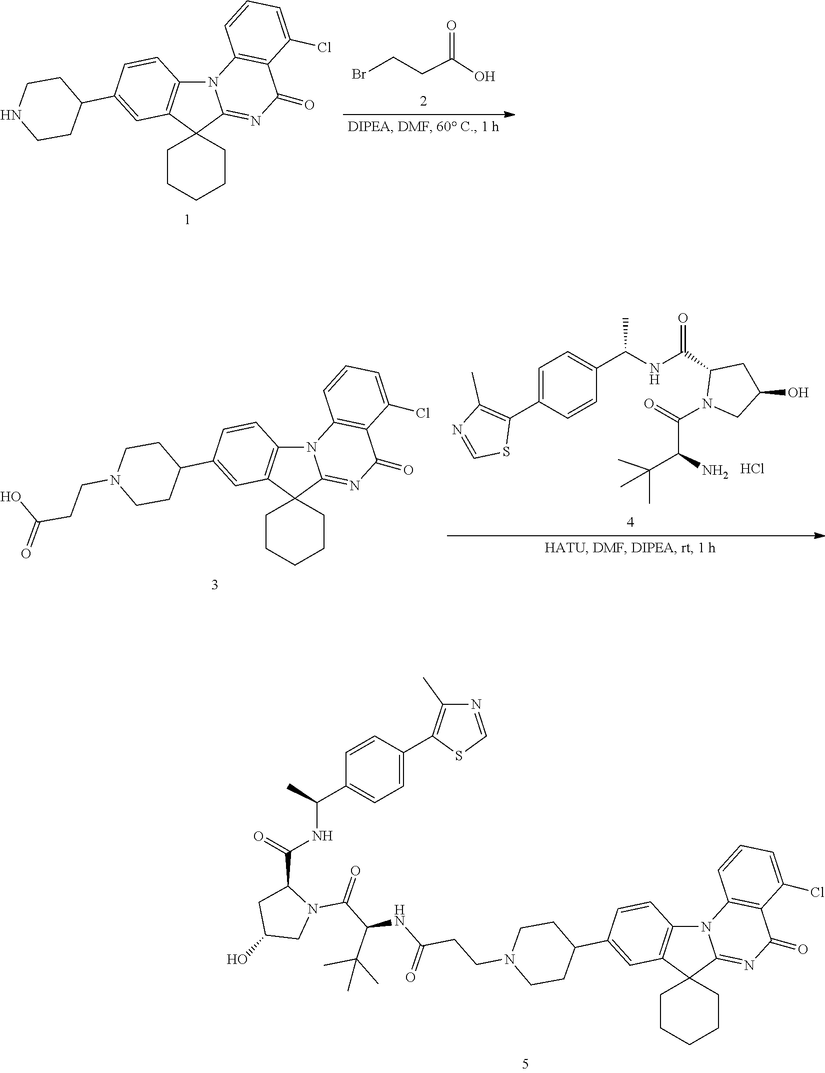

Preparation of 3. To a solution of 1 (50 mg, 0.12 mmol, 1.0 equiv.) in DMF (2 mL) was added 2 (18.2 mg, 0.12 mmol, 0.1 equiv.) and DIPEA (46.2 mg, 0.36 mmol, 3.0 equiv.). The mixture was stirred for 1 h at 60° C. The residue was purified by C-18 reversal column (elution solvents: CH₃CN/H₂O from 10% to 100%, 0.1% TFA) to get pure product (28 mg, 48%). ESI MS m/z: 492.20 [M+H]⁺.

Preparation of 5. To a solution of 3 (20 mg, 0.041 mmol, 1.0 equiv.), and 4 (13.4 mg, 0.041 mmol, 1.0 equiv.) in DMF (2 mL) was added HATU (23.0 mg, 0.061 mmol, 1.5 equiv.) and DIPEA (15.7 mg, 0.12 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (19.0 mg, 52%). ESI MS m/z: 918.41 [M+H]⁺.

Compound A⁷. (2S,4R)-1-((S)-2-(4-(4-(4′-chloro-5′-oxo-5′H-spiro[cyclohexane-1,7′-indolo[1,2-a]quinazolin]-9′-yl) piperidin-1-yl) butanamido)-3,3-dimethylbutanoyl)-4-hydroxy-N—((S)-1-(4-(4-methylthiazol-5-yl)phenyl)ethyl) pyrrolidine-2-carboxamide

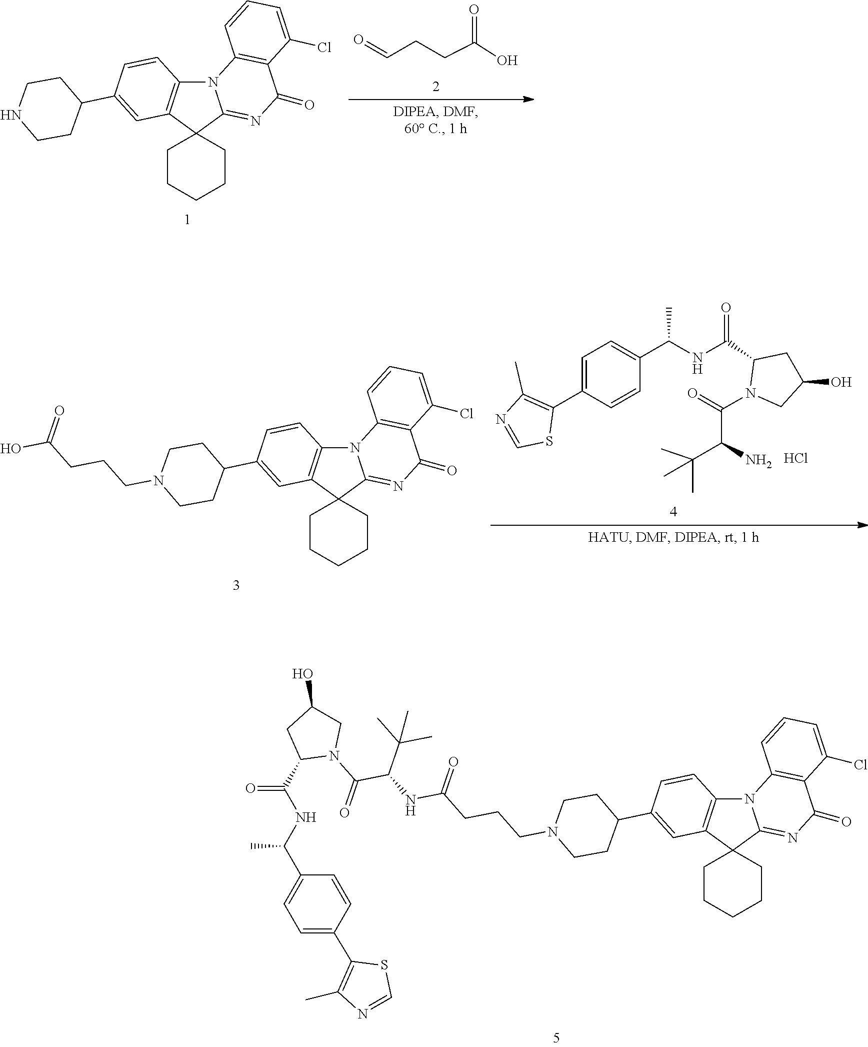

Preparation of 3. To a solution of 1 (50 mg, 0.12 mmol, 1 equiv.) in DCE/DMF (2/2 mL) was added 2 (18.2 mg, 0.18 mmol, 1.5 equiv.). The mixture was stirred for 15 min. then NaBH (OAc) 3 (75 mg, 0.36 mmol, 3 equiv.) was added. let the reaction mixture stirred for additional 15 min. After reaction, TFA (0.5 mL) was added to quench the reaction. The DCE was removed under rotatory evaporator. The residue was purified by C-18 reversal column (elution solvents: CH₃CN/H₂O from 10% to 100%, 0.1% TFA) to get pure product (52 mg, 86%). ESI MS m/z: 506.21 [M+H]⁺.

Preparation of 5. To a solution of 3 (20 mg, 0.040 mmol, 1.0 equiv.), and 4 (13.0 mg, 0.040 mmol, 1.0 equiv.) in DMF (2 mL) was added HATU (23.0 mg, 0.061 mmol, 1.5 equiv.) and DIPEA (15.3 mg, 0.12 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (19.0 mg, 51%). ESI MS m/z: 932.42 [M+H]⁺.

Compound A⁸. (2S,4R)-1-((S)-2-(5-(4-(4′-chloro-5′-oxo-5′H-spiro[cyclohexane-1,7′-indolo[1,2-a]quinazolin]-9′-yl) piperidin-1-yl) pentanamido)-3,3-dimethylbutanoyl)-4-hydroxy-N—((S)-1-(4-(4-methylthiazol-5-yl)phenyl)ethyl) pyrrolidine-2-carboxamide

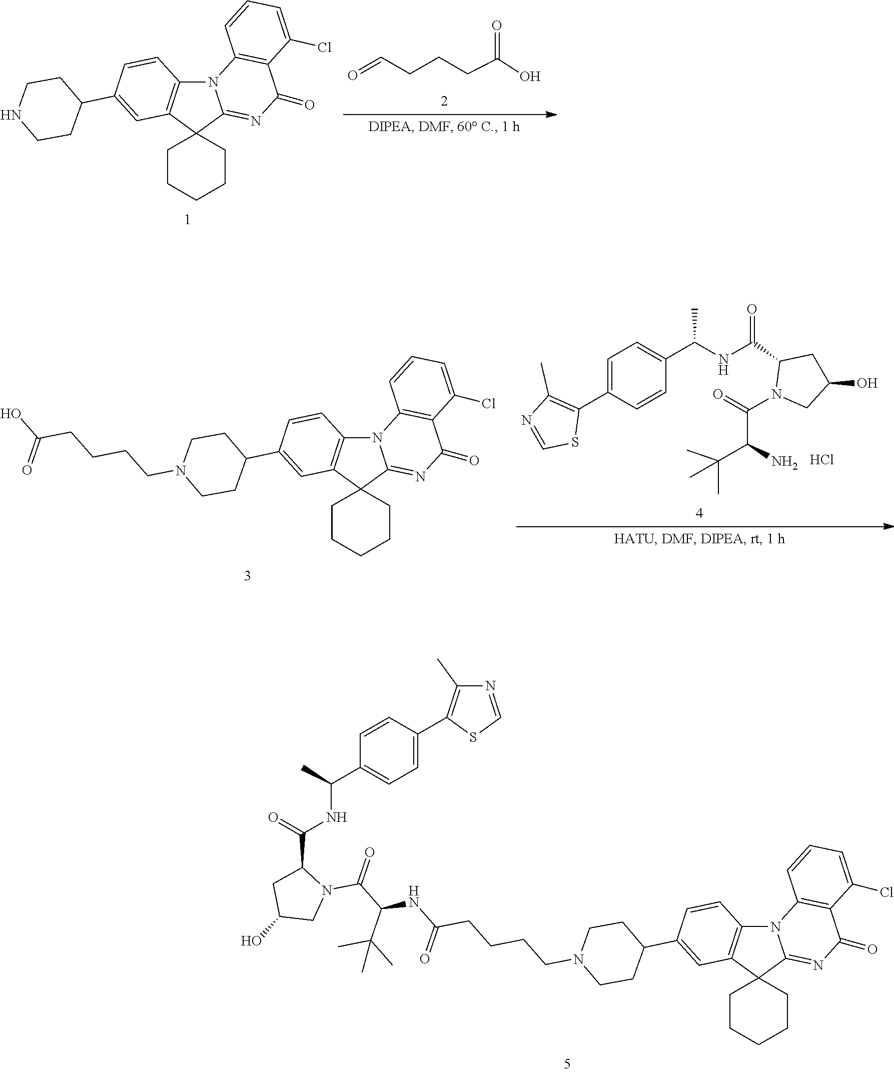

Preparation of 3. To a solution of 1 (50 mg, 0.12 mmol, 1 equiv.) in DCE/DMF (2/2 mL) was added 2 (20.7 mg, 0.18 mmol, 1.5 equiv.). The mixture was stirred for 15 min. then NaBH (OAc) 3 (75 mg, 0.36 mmol, 3 equiv.) was added. let the reaction mixture stirred for additional 15 min. After reaction, TFA (0.5 mL) was added to quench the reaction. The DCE was removed under rotatory evaporator. The residue was purified by C-18 reversal column (elution solvents: CH₃CN/H₂O from 10% to 100%, 0.1% TFA) to get pure product (40 mg, 65%). ESI MS m/z: 520.23 [M+H]⁺.

Preparation of 5. To a solution of 3 (20 mg, 0.038 mmol, 1.0 equiv.), and 4 (12.7 mg, 0.038 mmol, 1.0 equiv.) in DMF (2 mL) was added HATU (21.0 mg, 0.058 mmol, 1.5 equiv.) and DIPEA (15.0 mg, 0.12 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (22.0 mg, 60%). ESI MS m/z: 946.45 [M+H]⁺.

Compound A⁹. (2S,4R)-1-((S)-2-(6-(4-(4′-chloro-5′-oxo-5′H-spiro[cyclohexane-1,7′-indolo[1,2-a]quinazolin]-9′-yl) piperidin-1-yl) hexanamido)-3,3-dimethylbutanoyl)-4-hydroxy-N—((S)-1-(4-(4-methylthiazol-5-yl)phenyl)ethyl) pyrrolidine-2-carboxamide

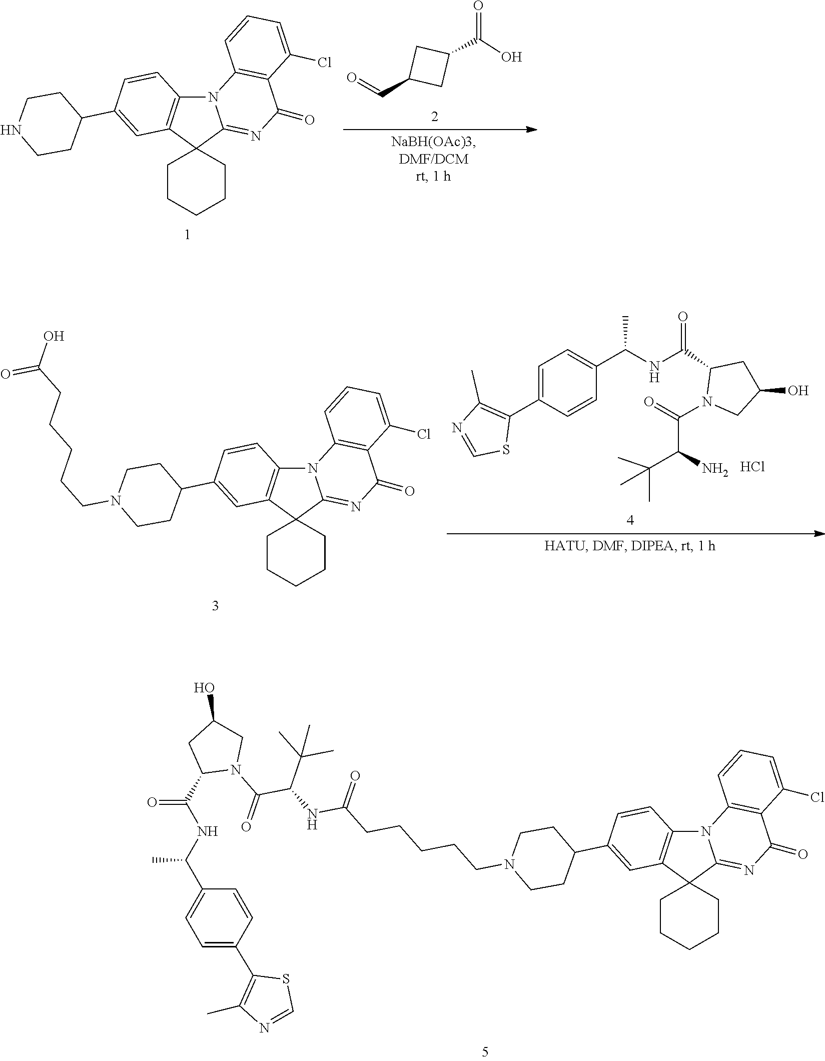

Preparation of 3. To a solution of 1 (50 mg, 0.12 mmol, 1 equiv.) in DCE/DMF (2/2 mL) was added 2 (20.7 mg, 0.18 mmol, 1.5 equiv.). The mixture was stirred for 15 min. then NaBH (OAc) 3 (75 mg, 0.36 mmol, 3 equiv.) was added. let the reaction mixture stirred for additional 15 min. After reaction, TFA (0.5 mL) was added to quench the reaction. The DCE was removed under rotatory evaporator. The residue was purified by C-18 reversal column (elution solvents: CH₃CN/H₂O from 10% to 100%, 0.1% TFA) to get pure product (36 mg, 58%). ESI MS m/z: 534.24 [M+H]⁺.

Preparation of 5. To a solution of 3 (20 mg, 0.037 mmol, 1.0 equiv.), and 4 (12.4 mg, 0.037 mmol, 1.0 equiv.) in DMF (2 mL) was added HATU (21.0 mg, 0.056 mmol, 1.5 equiv.) and DIPEA (14.5 mg, 0.11 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (18.0 mg, 48%). ESI MS m/z: 960.45 [M+H]⁺.

Compound A¹⁰. (2S,4R)—N—((S)-5-(4-(4-chloro-5-oxo-2′,3′,5′,6′-tetrahydro-5H-spiro[indolo[1,2-a]quinazoline-7,4′-pyran]-10-yl) piperidin-1-yl)-1-(4-(4-methylthiazol-5-yl)phenyl) pentyl)-1-((S)-2-(1-fluorocyclopropane-1-carboxamido)-3,3-dimethylbutanoyl)-4-hydroxypyrrolidine-2-carboxamide

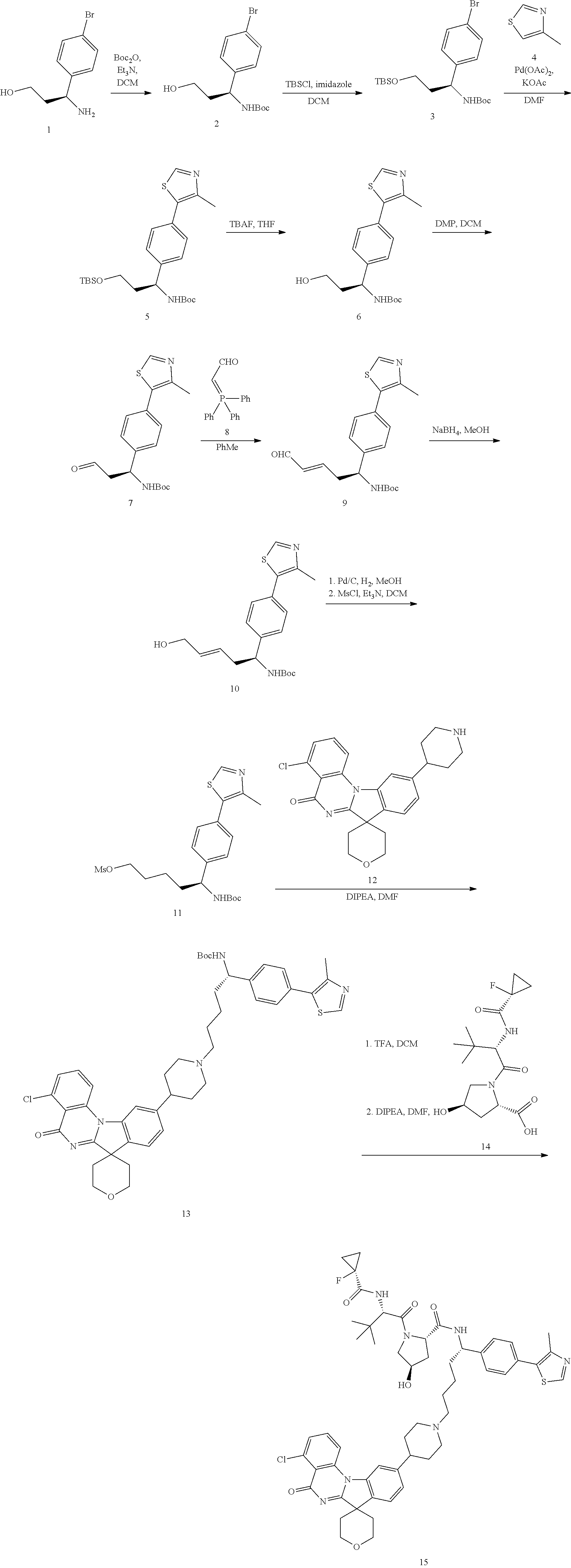

Step 1: To a flask was added(S)-3-amino-3-(4-bromophenyl) propan-1-ol (5.0 g, 21.7 mmol, 1.0 eq) and di-tert-butyl dicarbonate (5.0 g, 22.9 mmol, 1.05 eq) in DCM (80 mL). The suspension was stirred at room temperature for 5 minutes and triethylamine (3.3 mL, 23.8 mmol, 1.1 eq) was added. The reaction mixture was stirred overnight and was diluted with water (100 mL). The organic phase was separated, washed with 1N HCl (50 mL), saturated sodium bicarbonate solution (50 mL) and brine (50 mL), then dried over Na₂SO₄. The solvent was removed and the residue was purified by column chromatography (0-50% Ethyl acetate in hexanes) to give compound 2 (6.13 g, 85%).

Step 2: To a flask was added compound 2 (1.0 g, 3.0 mmol, 1.0 eq) and imidazole (268 mg, 3.94 mmol, 1.3 eq). DMF (1.5 mL) was added to the flask and the mixture was stirred at room temperature until fully dissolved, tert-Butyldimethylsilyl chloride (548 mg, 3.63 mmol, 1.2 eq) was added and the reaction mixture was stirred for 2 hours. Water (20 mL) was added and the mixture was extracted with EtOAc (20 mL) 3 times. The combined organic layer was washed with water (40 mL) and brine (40 mL), dried over Na₂SO₄ and filtered. The solvent was removed and the residue was purified by column chromatography (0-25% Ethyl acetate in hexanes) to give compound 3 (1.18 g, 89%).

Step 3: To a flask was added compound 3 (1.18 g, 2.66 mmol, 1.0 eq), 4-methylthiazole (527 mg, 5.31 mmol, 2.0 eq), Pd (OAc) 2 (60 mg, 0.27 mmol, 0.1 eq) and KOAc (522 mg, 5.31 mmol, 2.0 eq). Dry DMF (5 mL) was added and the flask was flushed with nitrogen. The mixture was stirred at 90° C. for 12 h. After cooling to room temperature, water (40 mL) and EtOAc (40 mL) was added and the mixture was filtered through celite. The mixture was extracted with EtOAc (40 mL) 2 times. The combined organic layer was washed with water (60 mL) and brine (60 mL), dried over Na₂SO₄ and filtered. The solvent was removed and the residue was purified by column chromatography (0-100% Ethyl acetate in hexanes) to give compound 5 (420 mg, 34%).

Step 4: To a flask was added compound 5 (420 mg, 0.91 mmol, 1.0 eq) and tetra-n-butylammonium fluoride (712 mg, 2.72 mmol, 3.0 eq). THF (10 mL) was added and the reaction mixture was stirred at room temperature for 3 hours. Water (10 mL) was added and the mixture was extracted with EtOAc (10 mL) 3 times. The combined organic layer was washed with water (20 mL) and brine (20 mL), dried over Na₂SO₄ and filtered. The solvent was removed and the residue was purified by column chromatography (0-100% Ethyl acetate in hexanes) to give compound 6 (291 mg, 92%).

Step 5: To a stirred solution of compound 6 (106 mg, 0.30 mmol, 1.0 eq) in DCM (3 mL) was added Dess-Martin periodinane (168 mg, 0.40 mmol, 1.3 eq). The reaction mixture was stirred at room temperature for 2 hours. Saturated Na₂S₂O₃ solution (10 mL) was added and stirred for 15 minutes. The mixture was extracted with DCM (5 mL) 3 times. The combined organic layer was washed with water (10 mL) and brine (10 mL), dried over MgSO₄ and filtered. The solvent was removed and the residue was purified by column chromatography (0-80% Ethyl acetate in hexanes) to give compound 7 (44 mg, 42%).

Step 6: To a flask was added compound 7 (44 mg, 0.13 mmol, 1.0 eq) in toluene (1 mL) and the mixture was heated to 85° C. (Triphenylphosphoranylidene) acetaldehyde (50 mg, 0.16 mmol, 1.3 eq) was added and the reaction mixture was stirred for 5 hours. Solvent was removed and the residue was purified by column chromatography (0-80% Ethyl acetate in hexanes) to give compound 9 (35 mg, 75%).

Step 7: To a solution of compound 9 (17 mg, 0.045 mmol, 1.0 eq) in MeOH (1 mL) was added sodium borohydride (2.1 mg, 0.055 mmol, 1.2 eq). The mixture was stirred at room temperature for 4 hours and then saturated ammonium chloride solution (2 mL) was added and the mixture was extracted with EtOAc (2 mL) 3 times. The the combined organic layer was washed with water (5 mL) and saturated brine (5 mL), dried over Na₂SO₄ and filtered. Solvent was removed and the residue was purified by column chromatography (0-100% Ethyl acetate in hexanes) to give compound 10 (16 mg, 95%).

Step 8: To a stirred solution of compound 10 (33 mg, 0.088 mmol, 1.0 eq) in methanol (1.5 mL) was added palladium on carbon (3.3 mg, 10% wt). The flask was flushed with hydrogen and the reaction mixture was stirred at room temperature for 12 h. The mixture was filtered by celite and solvent was removed to give crude product. The crude product (15 mg, 0.040 mmol) was dissolved in DCM (0.5 mL). Methanesulfonyl chloride (9.2 mg, 0.080 mmol, 2.0 eq) was added dropwise at 0° C. The mixture was stirred for 5 minutes and triethylamine (0.017 mL, 0.12 mmol, 3.0 eq) was added and the reaction mixture was stirred at room temperature for 1 hour. Solvent was removed and the residue was purified by column chromatography (0-80% Ethyl acetate in hexanes) to give compound 11 (16 mg, 88%).

Step 9: To a solution of compound 11 (8.5 mg, 0.019 mmol, 1.0 eq) and compound 12 (10 mg, 0.018 mmol, 1.0 eq) in DMF (0.5 mL) was added DIPEA (0.010 mL, 0.057 mmol, 3.0 eq). The mixture was stirred at 80° C. for 12 hours. After cooling to room temperature, the mixture was subjected to reverse phase flash chromatography (10-100% MeCN in H₂O, 0.1% TFA) to give compound 13 (9 mg, 61%).

Step 10: Compound 13 (9 mg, 0.012 mmol, 1.0 eq) was dissolved in DCM (0.5 mL) and TFA (0.5 mL) and the mixture was stirred at room temperature for 1 hour. Solvent was removed and the residue was dissolved in DMF (0.5 mL). Compound 14 (4.2 mg, 0.0127 mmol, 1.1 eq) and HATU (4.8 mg, 0.0127 mmol, 1.1 eq) was added and the mixture was stirred at room temperature for 5 minutes, followed by adding DIPEA (0.010 mL, 0.064 mmol, 5.3 eq) and the reaction mixture was stirred for another 30 minutes. The mixture was subjected to Prep-HPLC (25-100% MeCN in H₂O, 0.1% TFA) to give compound 15 (2.7 mg, 23%).

### Compound A11. (2S,4R)-1-((S)-2-((1r,4S)-4-((4-(4′-chloro-5′-oxo-5′H-spiro[cyclohexane-1,7′-indolo[1,2-a]quinazolin]-9′-yl)piperidin-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide

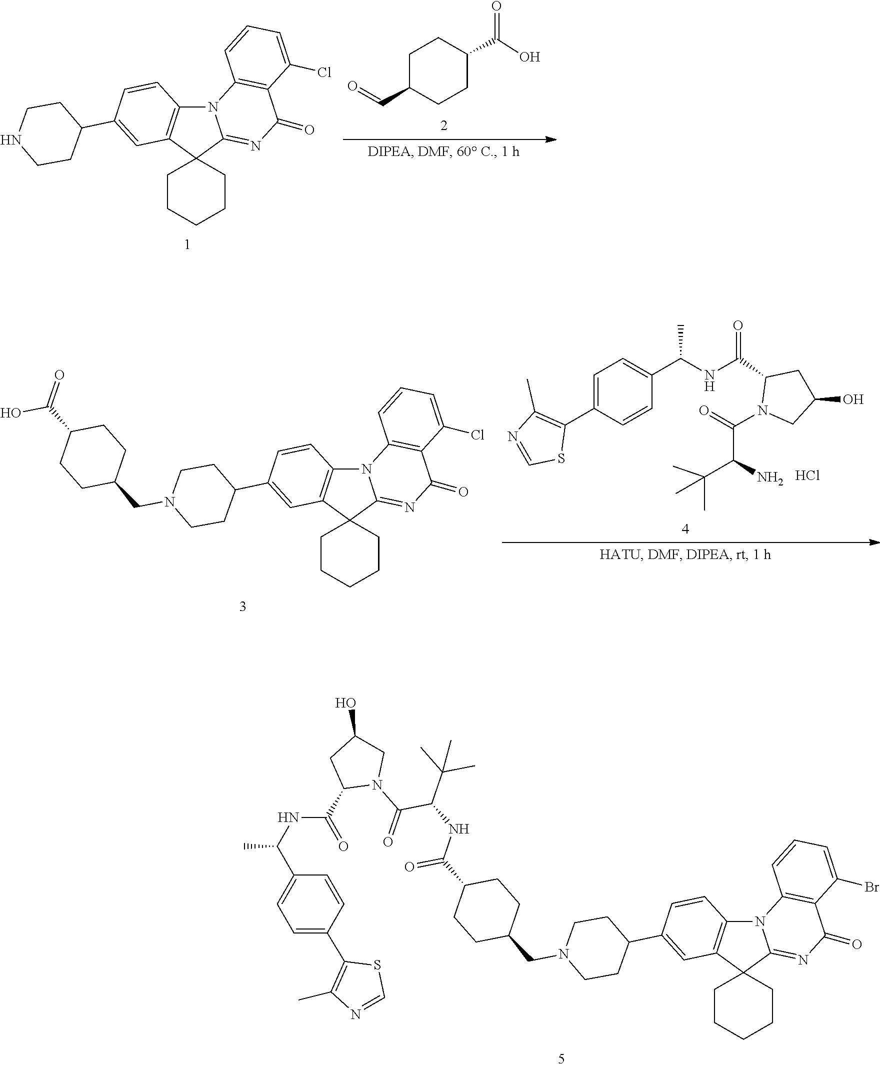

Preparation of 3. To a solution of 1 (50 mg, 0.12 mmol, 1 equiv.) in DCE/DMF (2/2 mL) was added 2 (28.0 mg, 0.18 mmol, 1.5 equiv.). The mixture was stirred for 15 min, then NaBH(OAc)₃ (75 mg, 0.36 mmol, 3 equiv.) was added, let the reaction mixture stirred for additional 15 min. After reaction, TFA (0.5 mL) was added to quench the reaction. The DCE was removed under rotatory evaporator. The residue was purified by C-18 reversal column (elution solvents: CH₃CN/H₂O from 10% to 100%, 0.1% TFA) to get pure product (46 mg, 69%). ESI MS m/z: 560.26 [M+H]⁺.

Preparation of 5. To a solution of 3 (20 mg, 0.036 mmol, 1.0 equiv.), and 4 (11.8 mg, 0.036 mmol, 1.0 equiv.) in DMF (2 mL) was added HATU (20.4 mg, 0.054 mmol, 1.5 equiv.) and DIPEA (13.8 mg, 0.11 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (21.0 mg, 60%). ESI MS m/z: 986.45 [M+H]⁺.

### Compound A12. (2S,4R)-1-((S)-2-((1r,4S)-4-((3-(4-(4′-chloro-5′-oxo-5′H-spiro[cyclohexane-1,7′-indolo[1,2-a]quinazolin]-9′-yl)piperidin-1-yl) azetidin-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide

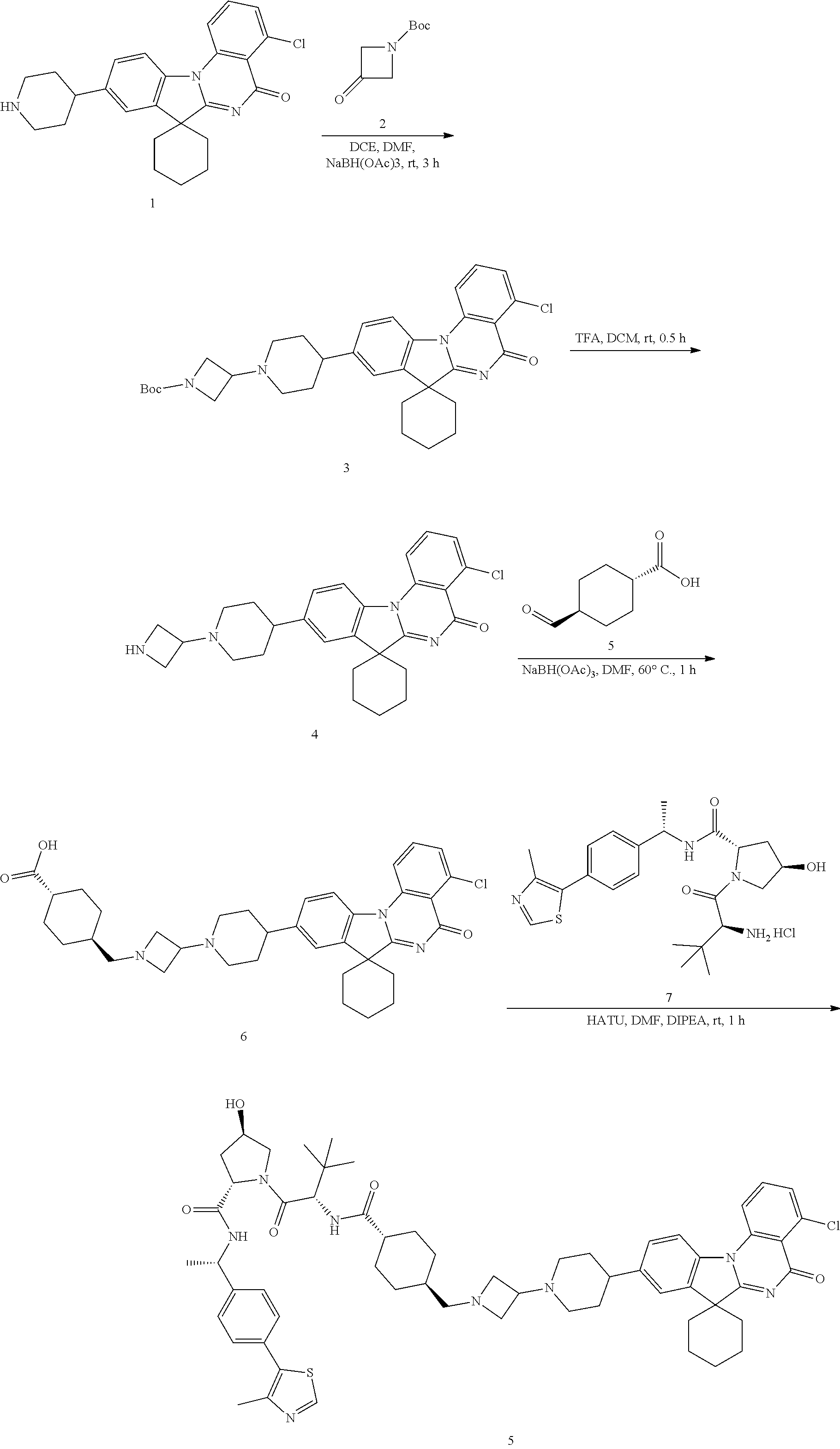

Preparation of 3. To a solution of 1 (50 mg, 0.12 mmol, 1 equiv.) in DCE/DMF (2/2 mL) was added 2 (44.8 mg, 0.24 mmol, 2.0 equiv.). The mixture was stirred for 15 min, then NaBH(OAc)₃ (126 mg, 0.60 mmol, 5 equiv.) was added, let the reaction mixture stirred for additional 3 h. After reaction, TFA (0.5 mL) was added to quench the reaction. The DCE was removed under rotatory evaporator. The residue was purified by C-18 reversal column (elution solvents: CH₃CN/H₂O from 10% to 100%, 0.1% TFA) to get pure product (12 mg, 18%). ESI MS m/z: 575.27 [M+H]⁺.

Preparation of 4. To a solution of 3 (12 mg, 0.021 mmol, 1 equiv.) in DCM (3 mL) was added TFA (3 mL). The mixture was stirred for 30 min. After reaction, the DCM and TFA were removed under rotatory evaporator. The residue was used in the next step directly. ESI MS m/z: 475.22 [M+H]⁺.

Preparation of 6. To a solution of crude 4 in DCE/DMF (2/2 mL) was added 5 (5.8 mg, 0.036 mmol, 1.5 equiv.). The mixture was stirred for 15 min, then NaBH(OAc)₃ (15.6 mg, 0.073 mmol, 3 equiv.) was added, let the reaction mixture stirred for additional 3 h. After reaction, TFA (0.2 mL) was added to quench the reaction. The DCE was removed under rotatory evaporator. The residue was purified by C-18 reversal column (elution solvents: CH₃CN/H₂O from 10% to 100%, 0.1% TFA) to get pure product (8.0 mg, 53%). ESI MS m/z: 615.30 [M+H]⁺.

Preparation of 8. To a solution of crude 6 (8.0 mg, 0.013 mmol, 1.0 equiv.), and 7 (4.3 mg, 0.013 mmol, 1.0 equiv.) in DMF (2 mL) was added HATU (7.4 mg, 0.020 mmol, 1.5 equiv.) and DIPEA (5.4 mg, 0.039 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (7.0 mg, 52%). ESI MS m/z: 1041.51 [M+H]⁺.

### Compound A13. (2S,4R)-1-((S)-2-((1r,4S)-4-((4-(4′-chloro-5′-oxo-5′H-spiro[cyclohexane-1,7′-indolo[1,2-a]quinazolin]-9′-yl)-[1,4′-bipiperidin]-1′-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide

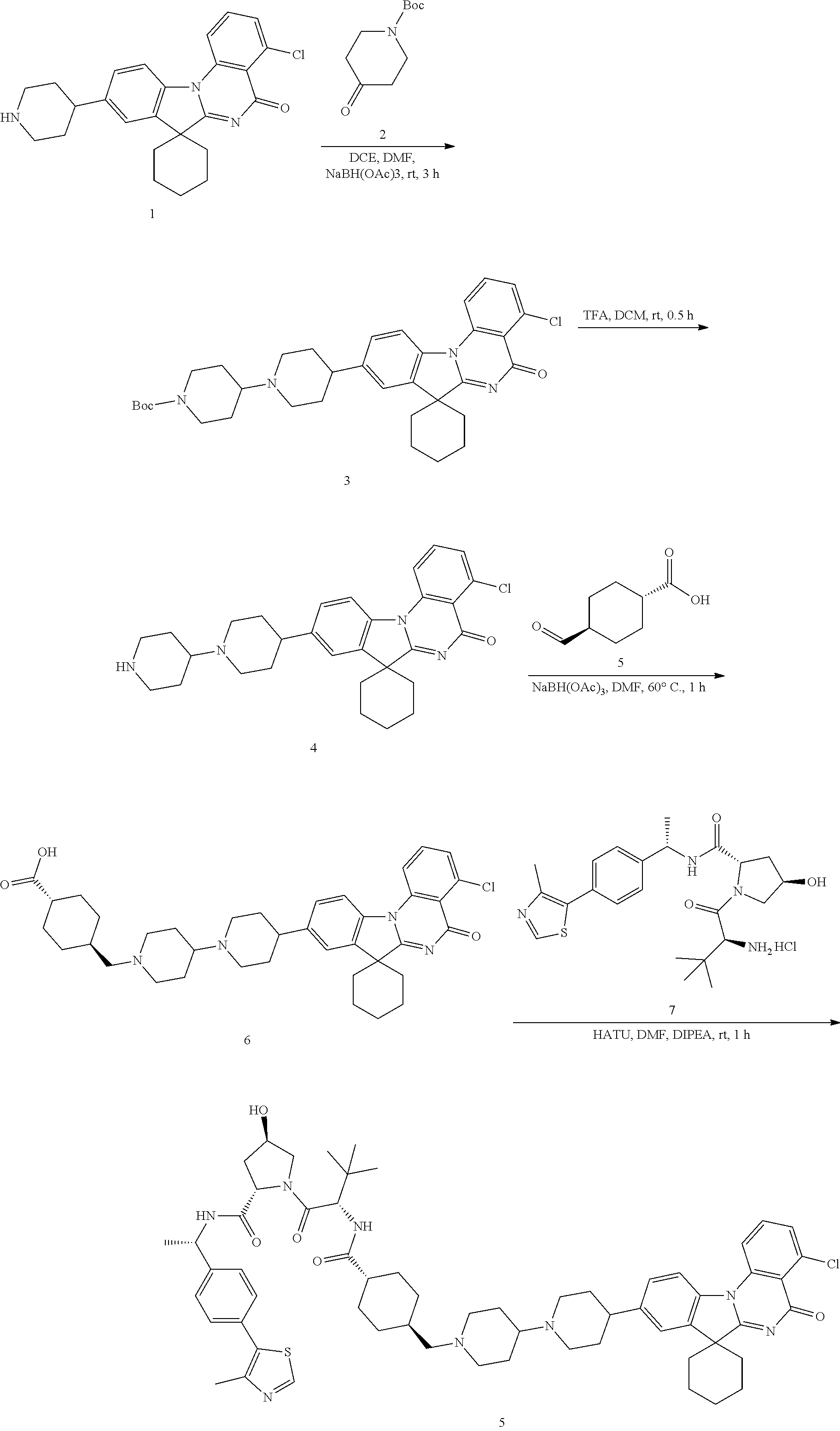

Preparation of 3. To a solution of 1 (50 mg, 0.12 mmol, 1 equiv.) in DCE/DMF (2/2 mL) was added 2 (44.8 mg, 0.24 mmol, 2.0 equiv.). The mixture was stirred for 15 min, then NaBH(OAc)₃ (151 mg, 0.71 mmol, 6 equiv.) was added, let the reaction mixture stirred for additional 3 h. After reaction, TFA (0.5 mL) was added to quench the reaction. The DCE was removed under rotatory evaporator. The residue was purified by C-18 reversal column (elution solvents: CH₃CN/H₂O from 10% to 100%, 0.1% TFA) to get pure product (8 mg, 11%). ESI MS m/z: 603.30 [M+H]⁺.

Preparation of 4. To a solution of 3 (8 mg, 0.013 mmol, 1 equiv.) in DCM (3 mL) was added TFA (3 mL). The mixture was stirred for 30 min. After reaction, the DCM and TFA were removed under rotatory evaporator. The residue was used in the next step directly. ESI MS m/z: 503.25 [M+H]⁺.

Preparation of 6. To a solution of crude 4 in DCE/DMF (2/2 mL) was added 5 (3.1 mg, 0.020 mmol, 1.5 equiv.). The mixture was stirred for 15 min, then NaBH(OAc)₃ (8.5 mg, 0.040 mmol, 3 equiv.) was added, let the reaction mixture stirred for additional 3 h. After reaction, TFA (0.2 mL) was added to quench the reaction. The DCE was removed under rotatory evaporator. The residue was purified by C-18 reversal column (elution solvents: CH₃CN/H₂O from 10% to 100%, 0.1% TFA) to get pure product (6.0 mg, 74%). ESI MS m/z: 643.33 M+H]⁺.

Preparation of 8. To a solution of crude 6 (8.0 mg, 0.009 mmol, 1.0 equiv.), and 7 (3.1 mg, 0.009 mmol, 1.0 equiv.) in DMF (2 mL) was added HATU (5.3 mg, 0.0013 mmol, 1.5 equiv.) and DIPEA (3.6 mg, 0.028 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (6.0 mg, 60%). ESI MS m/z: 1069.54 [M+H]⁺.

### Compound A14. (2S,4R)-1-((S)-2-((1r,4S)-4-((4-(4′-bromo-5′-oxo-5′H-spiro[cyclohexane-1,7′-indolo[1,2-a]quinazolin]-9′-yl)piperidin-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide

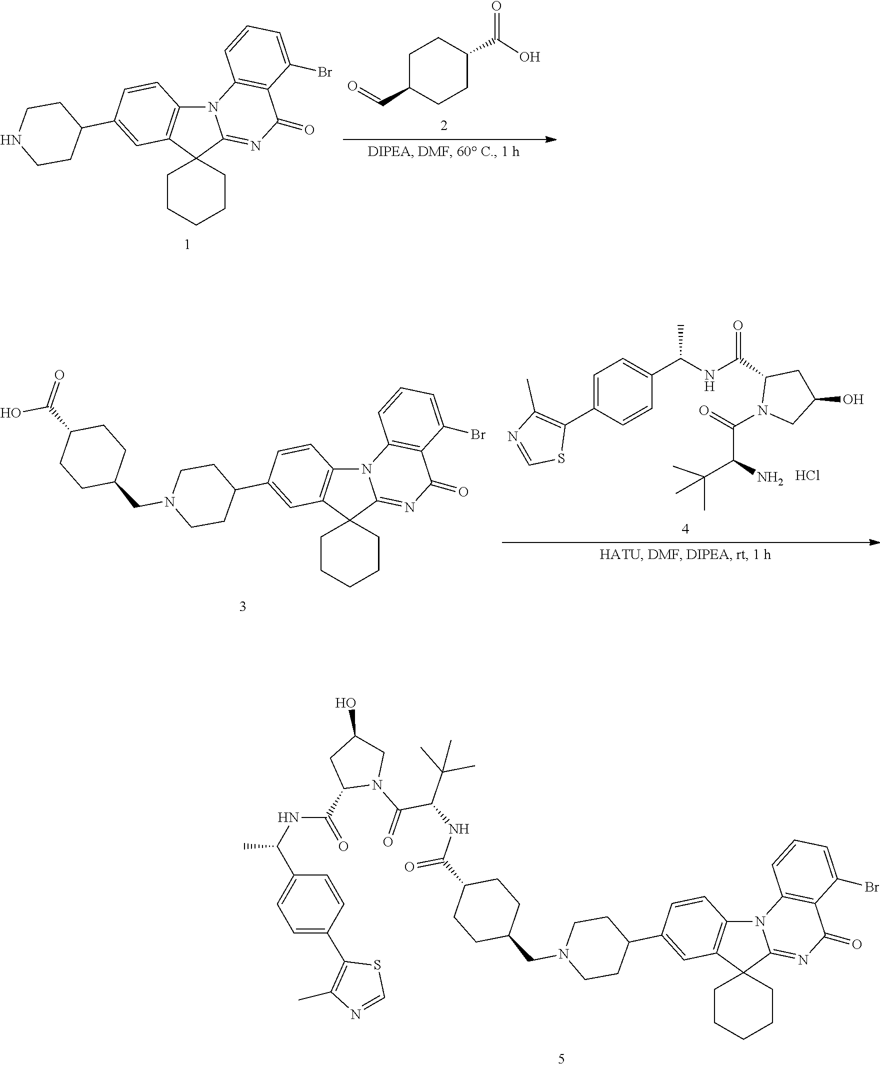

Preparation of 3. To a solution of 1 (50 mg, 0.111 mmol, 1 equiv.) in DCE/DMF (2/2 mL) was added 2 (25.2 mg, 0.16 mmol, 1.5 equiv.). The mixture was stirred for 15 min, then NaBH(OAc)₃ (68.5 mg, 0.32 mmol, 3 equiv.) was added, let the reaction mixture stirred for additional 15 min. After reaction, TFA (0.5 mL) was added to quench the reaction. The DCE was removed under rotatory evaporator. The residue was purified by C-18 reversal column (elution solvents: CH₃CN/H₂O from 10% to 100%, 0.1% TFA) to get pure product (46 mg, 71%). ESI MS m/z: 604.21 [M+H]⁺.

Preparation of 5. To a solution of 3 (20 mg, 0.033 mmol, 1.0 equiv.), and 4 (10.4 mg, 0.033 mmol, 1.0 equiv.) in DMF (2 mL) was added HATU (19.0 mg, 0.050 mmol, 1.5 equiv.) and DIPEA (12.8 mg, 0.099 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (26.0 mg, 76%). ESI MS m/z: 1032.42 [M+H]⁺. ¹H NMR (400 MHz, MeOD) δ 9.00 (d, J=2.0 Hz, 1H), 8.45 (d, J=8.6 Hz, 1H), 8.11 (d, J=8.2 Hz, 1H), 7.91 (d, J=7.9 Hz, 1H), 7.79-7.73 (m, 2H), 7.50-7.42 (m, 4H), 5.04 (dd, J=7.4, 5.3 Hz, 1H), 4.67-4.62 (m, 1H), 4.59 (t, J=8.3 Hz, 1H), 4.46 (m, 1H), 3.88 (d, J=11.1 Hz, 1H), 3.78 (dd, J=11.1, 4.0 Hz, 2H), 3.18 (d, J=3.3 Hz, 1H), 3.09 (d, J=6.7 Hz, 2H), 2.51 (d, J=3.3 Hz, 3H), 2.30-1.72 (m, 24H), 1.53 (d, J=7.0 Hz, 3H), 1.21 (t, J=12.9 Hz, 2H), 1.07 (s, 9H).

### Compound A15. (2S,4R)-1-((S)-2-((1r,4S)-4-((4-(4′-bromo-5′-oxo-5′H-spiro[cyclohexane-1,7′-indolo[1,2-a]quinazolin]-9′-yl)piperidin-1-yl)methyl)cyclohexane-1-carboxamido)-3,3-dimethylbutanoyl)-N—((R)-2-(dimethylamino)-1-(4-(4-methylthiazol-2-yl)phenyl)ethyl)-4-hydroxypyrrolidine-2-carboxamide

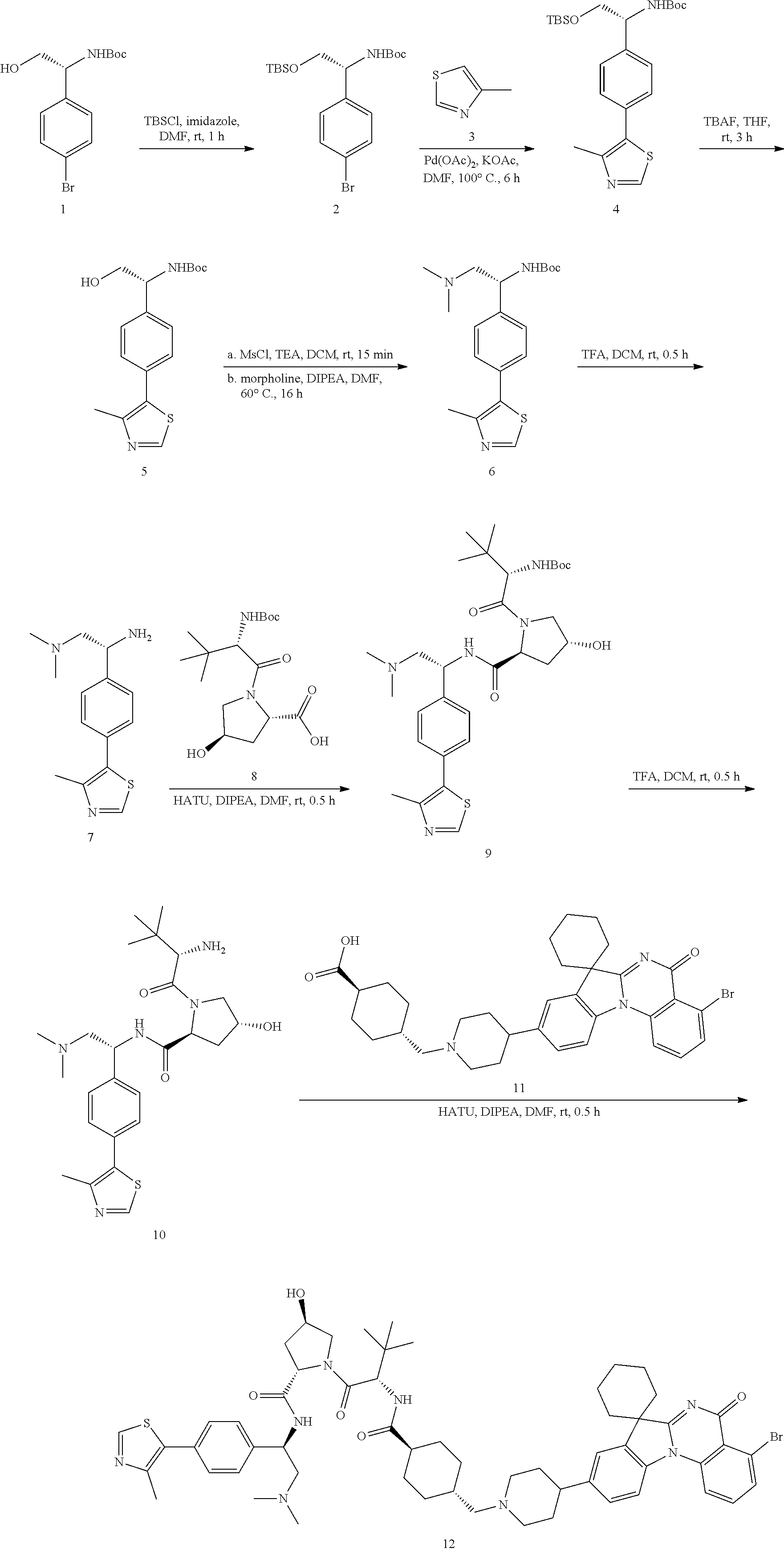

Preparation of 3. To a solution of 1 (10 g, 31.6 mmol, 1 equiv.) in DCE (120 mL) was added TBSCl (5.24 mg, 37.8 mmol, 1.1 equiv.) and imidazole (4.31, 63.25 mmol, 2 equiv.). The mixture was stirred for 1 h. After reaction, the mixture was washed by brine once, dried by Na₂SO₄. After removing solvent, the residue was purified by silica gel column chromatography (elution solvents: ethyl acetate/hexane=1/3) to get pure product (13 g, 95%). ESI MS m/z: 432.13 [M+H]⁺.

Preparation of 4. To a solution of 2 (13.0 g, 30.2 mmol, 1 equiv.) and 3 (6.00 g, 60.4 mmol, 2.0 equiv.) in DMF (80 mL) was added Pd (OAc) 2 (678 mg, 3.02 mmol, 0.1 equiv.) and KOAc (5.93 g, 60.4 mmol, 2 equiv). The mixture was stirred for 6 h at 120° C. Water (150 mL) was added to quench the reaction and product was extracted with ethyl acetate twice (100 mL×2). Combined organic phases were washed by brine twice, dried by anhydrous Na₂SO₄. After removing solvent, the received crude product was purified by silica gel column chromatography (ethyl acetate/hexane=1/1) to get pure product (12.0 g, 87%). ESI MS m/z: 449.22 [M+H]⁺.

Preparation of 5. To a solution of 4 (12 g, 26.7 mmol, 1 equiv.) in THF (120 mL) was added TBAF (80 mL, 80.23 mmol, 3.0 equiv.). The mixture was stirred for 3 h. After reaction, the solvent was removed and the residue was dissolved into ethyl acetate, washed by water twice, dried by Na₂SO₄. After removing solvent, the residue was purified by silica gel column chromatography (elution solvents: ethyl acetate/hexane=1/1) to get pure product (8.0 g, 89%). ESI MS m/z: 335.14 [M+H]⁺.

Preparation of 6. To a solution of 5 (800 mg, 2.39 mmol, 1 equiv.) in DCM (30 mL), was added TEA (480 mg, 4.88 mmol, 2 equiv.) and then MsCl (301 mg, 2.63 mmol, 1.1 equiv.) at room temperature. The mixture was stirred 15 min. After reaction, then a saturated Na₂HCO₃ solution was add and the mixture was stirred for 10 minutes. The DCM phase was collected and dried by anhydrous Na₂SO₄. After removing solvent, the residue was dissolved into DMF (10 mL), dimethylamine (320 mg, 7.18 mmol, 3 equiv.) and DIPEA (620 mg, 4.78 mmol, 2 equiv.) were added. The mixture was stirred for 16 h at 60° C. Water (30 mL) was added to quench the reaction and product was extracted with ethyl acetate twice (30 mL×2). Combined organic phases were washed by brine twice, dried by anhydrous Na₂SO₄. After removing solvent, the residue was purified by C-18 reversal column (elution solvents: CH₃CN/H₂O from 10% to 100%, 0.1% TFA) to get pure product (350 mg, 40%). ESI MS m/z: 362.18 [M+H]⁺.

Preparation of 7. To a solution of 6 (50 mg, 0.14 mmol, 1 equiv.) in DCM (3 mL), was added TEA (3 mL) and the mixture was stirred 30 min. After removing solvent, the residue was received and can be used in the next step directly. ESI MS m/z: 262.13 [M+H]⁺.

Preparation of 9. To a solution of crude 7 and 8 (47.6 mg, 0.14 mmol, 1.0 equiv.) in DMF (2 mL) was added HATU (79.0 mg, 0.21 mmol, 1.5 equiv.) and DIPEA (54.0 mg, 0.41 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (62.0 mg, 76%). ESI MS m/z: 588.31 [M+H]⁺.

Preparation of 10. To a solution of 9 (62 mg, 0.13 mmol, 1 equiv.) in DCM (3 mL), was added TEA (3 mL) and the mixture was stirred 30 min. After removing solvent, the residue was received and can be used in the next step directly. ESI MS m/z: 488.26 [M+H]⁺.

Preparation of 12. To a solution of crude 10 and 11 (76.7 mg, 0.13 mmol, 1.0 equiv.) in DMF (3 mL) was added HATU (72.0 mg, 0.19 mmol, 1.5 equiv.) and DIPEA (49.0 mg, 0.38 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (68.0 mg, 50%). ESI MS m/z: 1075.46 [M+H]⁺.

### Compound A16. (2S,4R)-1-((S)-2-((1r,4S)-4-((4-(4′-bromo-5′-oxo-5′H-spiro[cyclohexane-1,7′-indolo[1,2-a]quinazolin]-9′-yl)piperidin-1-yl)methyl)cyclohexane-1-carboxamido)-3,3-dimethylbutanoyl)-4-hydroxy-N—((R)-1-(4-(4-methylthiazol-5-yl)phenyl)-2-morpholinoethyl)pyrrolidine-2-carboxamide

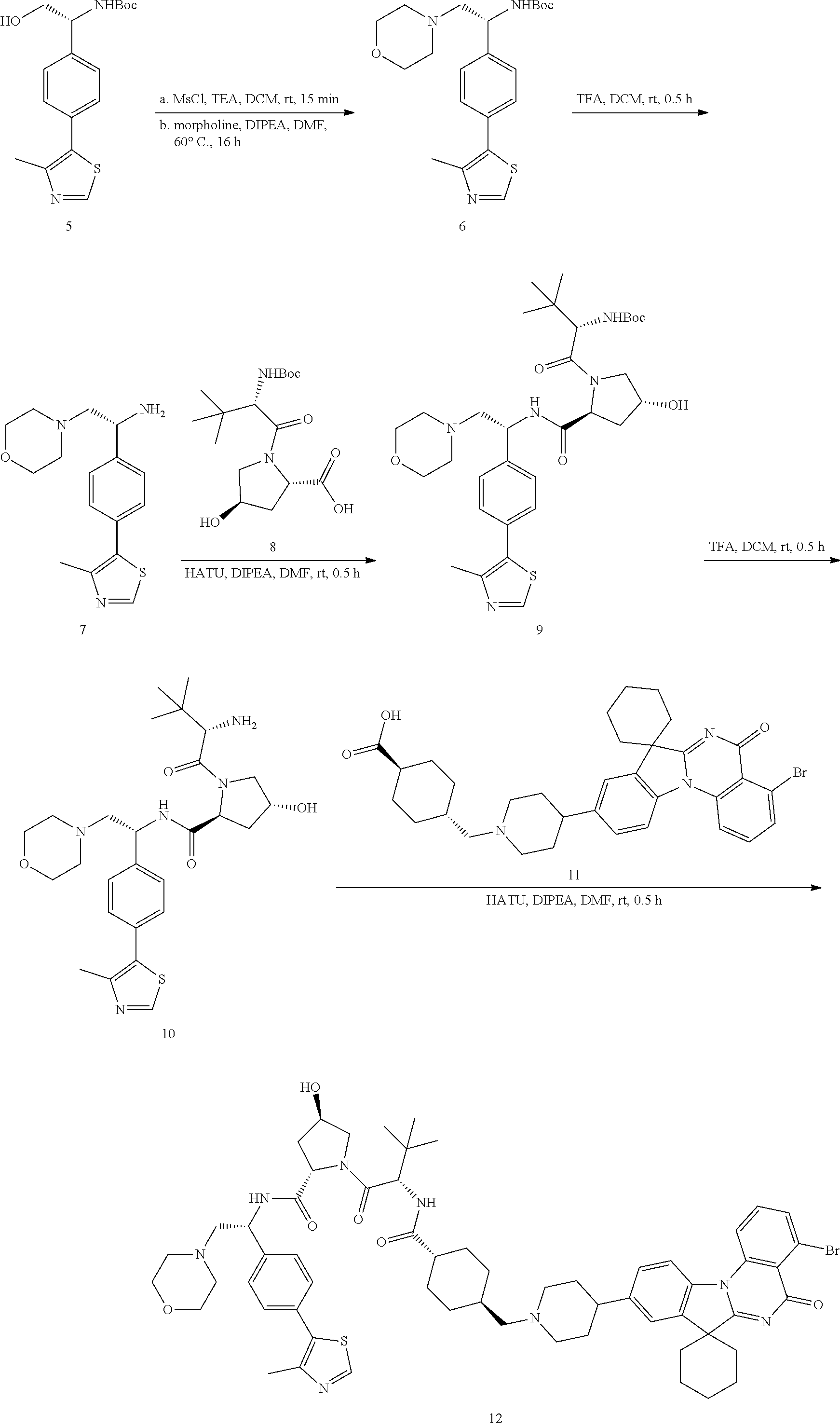

Preparation of 6. To a solution of 5 (800 mg, 2.39 mmol, 1 equiv.) in DCM (30 mL), was added TEA (480 mg, 4.88 mmol, 2 equiv.) and then MsCl (300 mg, 2.63 mmol, 1.1 equiv.) at room temperature. The mixture was stirred 15 min. After reaction, then a saturated Na₂HCO₃ solution was add and the mixture was stirred for 10 minutes. The DCM phase was collected and dried by anhydrous Na₂SO₄. After removing solvent, the residue was dissolved into DMF (10 mL), morpholine (615 mg, 7.18 mmol, 3 equiv.) and DIPEA (618 mg, 4.78 mmol, 2 equiv.) were added. The mixture was stirred for 16 h at 60° C. Water (30 mL) was added to quench the reaction and product was extracted with ethyl acetate twice (30 mL×2). Combined organic phases were washed by brine twice, dried by anhydrous Na₂SO₄. After removing solvent, the residue was purified by C-18 reversal column (elution solvents: CH₃CN/H₂O from 10% to 100%, 0.1% TFA) to get pure product (80 mg, 9%). ESI MS m/z: 404.19 [M+H]⁺.

Preparation of 7. To a solution of 6 (80 mg, 0.20 mmol, 1 equiv.) in DCM (3 mL), was added TEA (3 mL) and the mixture was stirred 30 min. After removing solvent, the residue was received and can be used in the next step directly. ESI MS m/z: 304.14 [M+H]⁺.

Preparation of 9. To a solution of crude 7 and 8 (68.6 mg, 0.20 mmol, 1.0 equiv.) in DMF (4 mL) was added HATU (113 mg, 0.30 mmol, 1.5 equiv.) and DIPEA (51.0 mg, 0.30 mmol, 2 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (70.0 mg, 56%). ESI MS m/z: 630.32 [M+H]⁺.

Preparation of 10. To a solution of 9 (70 mg, 0.11 mmol, 1 equiv.) in DCM (3 mL), was added TEA (3 mL) and the mixture was stirred 30 min. After removing solvent, the residue was received and can be used in the next step directly. ESI MS m/z: 530.27 [M+H]⁺.

Preparation of 12. To a solution of crude 10 and 11 (67.6 mg, 0.13 mmol, 1.0 equiv.) in DMF (3 mL) was added HATU (63.0 mg, 0.17 mmol, 1.5 equiv.) and DIPEA (43.0 mg, 0.33 mmol, 3 equiv.). The mixture was stirred for 1 h. Water (10 mL) was added to quench the reaction and product was extracted with ethyl acetate twice (10 mL×2). Combined organic phases were washed by brine twice, dried by anhydrous Na₂SO₄. After removing solvent, the received crude product was purified by pre-HPLC to get pure product (52.0 mg, 42%). ESI MS m/z: 1117.47 [M+H]⁺.

¹H NMR (400 MHz, CD3OD_SPE) δ 9.07 (s, 1H), 8.46 (dd, J=8.7, 1.0 Hz, 1H), 8.11 (d, J=8.6 Hz, 1H), 7.91 (dd, J=7.9, 0.9 Hz, 1H), 7.81-7.71 (m, 2H), 7.62-7.52 (m, 4H), 7.48 (dd, J=8.7, 1.8 Hz, 1H), 5.67 (dd, J=11.0, 3.9 Hz, 1H), 4.70-4.63 (m, 1H), 4.57 (dd, J=9.7, 7.3 Hz, 1H), 4.49 (s, 1H), 4.19-3.55 (m, 14H), 3.23-3.12 (m, 2H), 3.09 (d, J=6.4 Hz, 2H), 2.52 (s, 3H), 2.24-1.73 (m, 19H), 1.61 (s, 1H), 1.28-1.17 (m, 2H), 1.08 (s, 9H).

### Compound A17. (2S,4R)—N—((S)-6-(4-(4′-bromo-5′-oxo-5′H-spiro[cyclohexane-1,7′-indolo[1,2-a]quinazolin]-9′-yl)piperidin-1-yl)-1-(4-(4-methylthiazol-5-yl)phenyl) hexyl)-1-((S)-2-(1-(dimethylamino)cyclopropane-1-carboxamido)-3,3-dimethylbutanoyl)-4-hydroxypyrrolidine-2-carboxamide

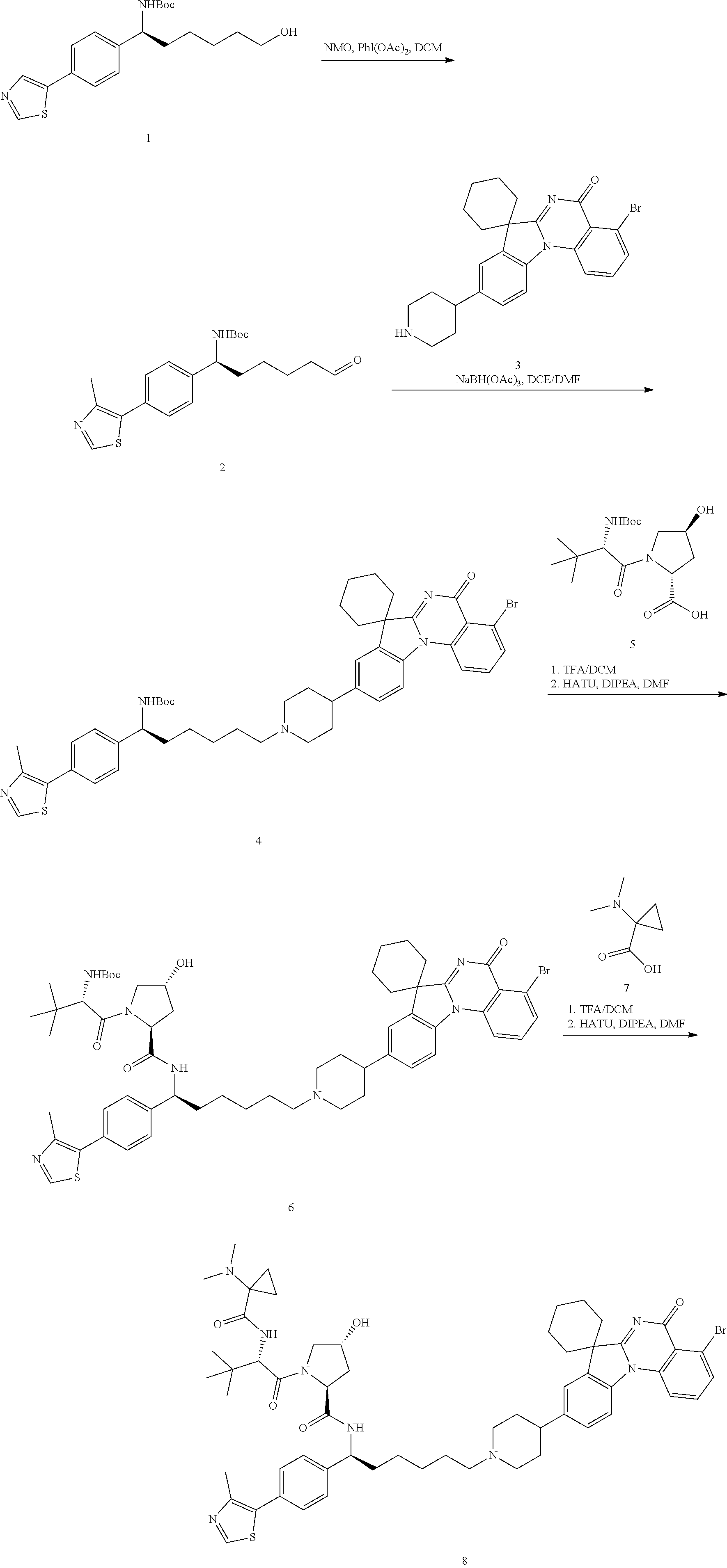

Step 1: To a stirred solution of compound 1 (210 mg, 0.54 mmol, 1.0 eq) in DCM (6 mL) was added (Diacetoxyiodo)benzene (225 mg, 0.70 mmol, 1.3 eq) and N-methylmorpholine N-oxide (17 mg, 0.11 mmol, 0.2 eq). The reaction mixture was stirred at room temperature for 2 hours. Saturated Na₂S₂O₃ solution (15 mL) was added and stirred for 15 minutes. The mixture was extracted with DCM (10 mL) 3 times. The combined organic layer was washed with water (20 mL) and brine (20 mL), dried over MgSO₄ and filtered. The solvent was removed and the residue was purified by column chromatography (0-80% Ethyl acetate in hexanes) to give compound 2 (155 mg, 74%).

Step 2: To a flask was added compound 2 (155 mg, 0.40 mmol, 1.0 eq), compound 3 (231 mmol, 0.40 mmol, 1.0 eq). DCE (2 mL) and DMF (2 mL) was added to the flask and the mixture was stirred at room temperature for 15 minutes. NaBH(OAc)₃ (254 mg, 1.2 mmol, 3.0 eq) was added and the reaction mixture was stirred for another 15 minutes. DCE was removed and the residue was subjected to reverse phase flash chromatography (10-100% MeCN in H₂O, 0.1% TFA) to give compound 4 (115 mg, 34%).

Step 3: Compound 4 (51.5 mg, 0.062 mmol, 1.0 eq) was dissolved in DCM (1 mL) and TFA (0.5 mL) and the reaction mixture was stirred at room temperature for 1 hour. Solvent was removed and the residue was dissolved in DMF (1 mL). Compound 5 (21 mg, 0.062 mmol, 1.0 eq) and HATU (26 mg, 0.068 mmol, 1.1 eq) was added the mixture was stirred for 5 minutes, followed by adding DIPEA (0.1 mL, 0.62 mmol, 10 eq) and the reaction mixture was stirred for another 30 minutes. The mixture was subjected to reverse phase flash chromatography (10-100% MeCN in H₂O, 0.1% TFA) to give compound 6 (53 mg, 81%).

Step 4: Compound 6 (26 mg, 0.023 mmol, 1.0 eq) was dissolved in DCM (1 mL) and TFA (0.5 mL) and the reaction mixture was stirred at room temperature for 1 hour. Solvent was removed and the residue was dissolved in DMF (1 mL). Compound 7 (3 mg, 0.023 mmol, 1.0 eq) and HATU (9.5 mg, 0.025 mmol, 1.1 eq) was added the mixture was stirred for 5 minutes, followed by adding DIPEA (0.035 mL, 0.23 mmol, 10 eq) and the reaction mixture was stirred for another 30 minutes. The mixture was subjected to Prep-HPLC (35-100% MeCN in H₂O, 0.1% TFA) to give compound 8 (16.3 mg, 67%). ¹H NMR (400 MHz, MeOD) δ 8.96 (s, 1H), 8.66 (d, J=8.1 Hz, 1H), 8.44 (dd, J=8.7, 1.0 Hz, 1H), 8.09 (d, J=8.6 Hz, 1H), 7.91 (dd, J=7.9, 0.9 Hz, 1H), 7.80-7.73 (m, 2H), 7.51-7.44 (m, 4H), 4.60 (dd, J=9.3, 7.6 Hz, 1H), 4.47 (d, J=4.8 Hz, 1H), 3.88 (d, J=11.2 Hz, 1H), 3.80-3.70 (m, 3H), 3.22-3.03 (m, 6H), 2.94 (s, 6H), 2.51 (s, 3H), 2.28-1.74 (m, 20H), 1.71-1.47 (m, 9H), 1.06 (d, 9H)

### Compound A18. (2S,4R)—N—((S)-6-(4-(4′-bromo-5′-oxo-5′H-spiro[cyclohexane-1,7′-indolo[1,2-a]quinazolin]-9′-yl)piperidin-1-yl)-1-(4-(4-methylthiazol-5-yl)phenyl) hexyl)-1-((S)-2-(1-((dimethylamino)methyl)cyclopropane-1-carboxamido)-3,3-dimethylbutanoyl)-4-hydroxypyrrolidine-2-carboxamide

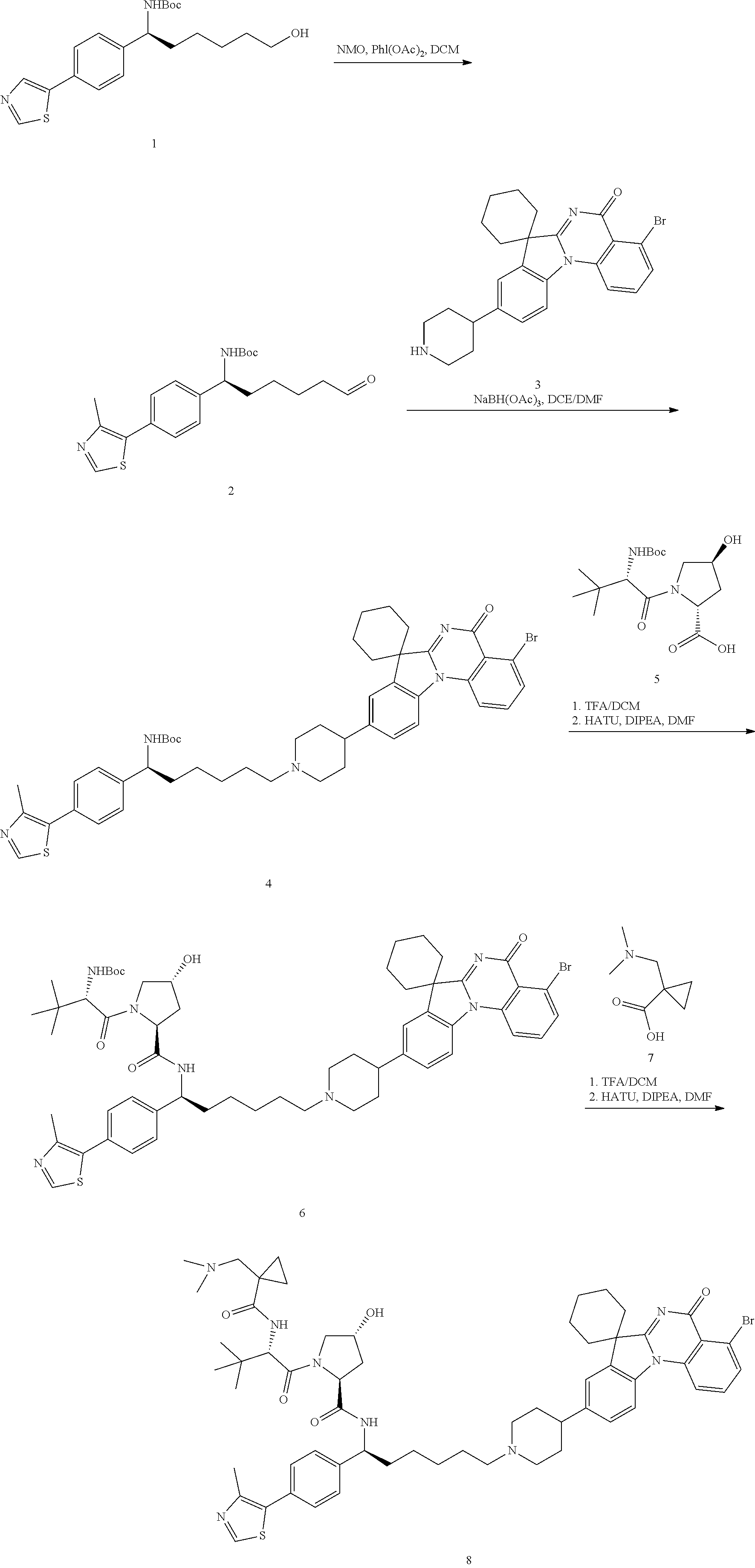

Compound A18 was synthesized using the similar procedures as used in the synthesis of Compound A17.

¹H NMR (400 MHz, MeOD) δ 8.97 (s, 1H), 8.44 (dd, J=8.7, 1.0 Hz, 1H), 8.09 (d, J=8.6 Hz, 1H), 7.90 (dd, J=7.8, 0.9 Hz, 1H), 7.83-7.71 (m, 2H), 7.53-7.38 (m, 5H), 4.74-4.67 (m, 1H), 4.60 (dd, J=9.4, 7.6 Hz, 1H), 4.48 (s, 1H), 3.93 (d, J=11.1 Hz, 1H), 3.80-3.70 (m, 3H), 3.41-3.29 (m, 2H), 3.22-3.03 (m, 5H), 2.91 (d, J=5.6 Hz, 6H), 2.51 (s, 3H), 2.29-1.74 (m, 20H), 1.65-1.36 (m, 6H), 1.20 (m, 3H), 1.05 (s, 9H).

### Compound A19 (2S,4R)-1-((S)-2-((1r,4S)-4-((4-(4′-bromo-5′-oxo-5′H-spiro[cyclohexane-1,7′-indolo[1,2-a]quinazolin]-9′-yl)piperidin-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide

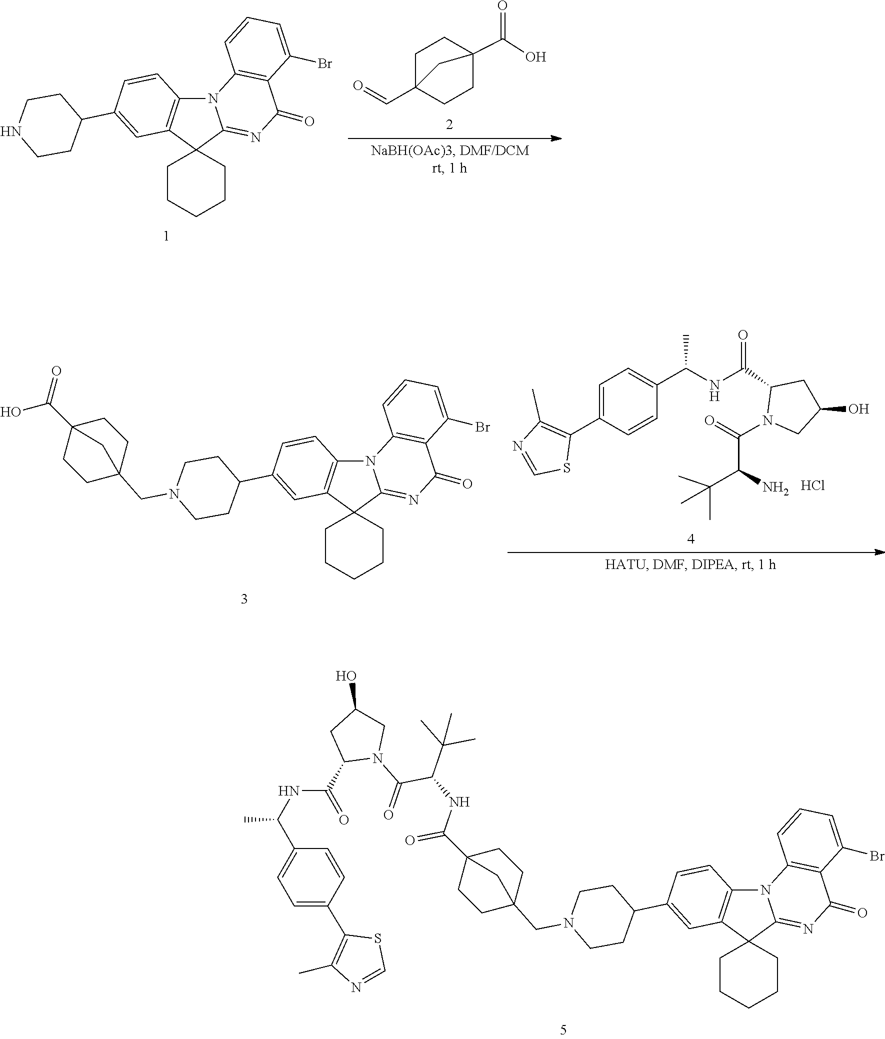

The synthetical procedure is similar to A¹¹. Target compound (12.2 mg) was obtained. ESI MS m/z: 1042.41 [M+H]⁺.

### Compound A20 (2S,4R)-1-((S)-2-(3-((4-(4′-bromo-5′-oxo-5′H-spiro[cyclohexane-1,7′-indolo[1,2-a]quinazolin]-9′-yl)piperidin-1-yl)methyl)cyclobutane-1-carboxamido)-3,3-dimethylbutanoyl)-4-hydroxy-N—((S)-1-(4-(4-methylthiazol-5-yl)phenyl)ethyl)pyrrolidine-2-carboxamide

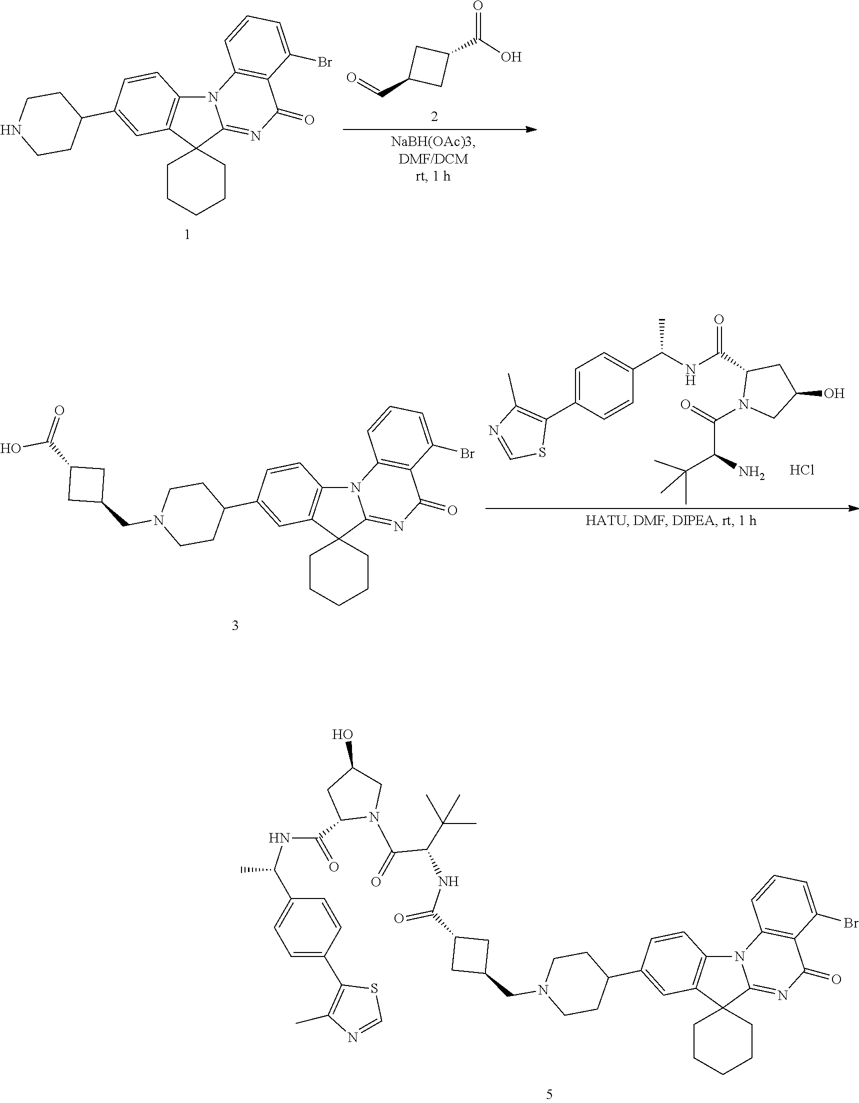

The synthetical procedure is similar to A¹¹. Target compound (8.6 mg) was obtained. ESI MS m/z: 1002.39 [M+H]⁺.

### Compound A21 (2S,4R)-1-((S)-2-(3-((4-(4′-bromo-5′-oxo-5′H-spiro[cyclohexane-1,7′-indolo[1,2-a]quinazolin]-9′-yl)piperidin-1-yl)methyl)cyclobutane-1-carboxamido)-3,3-dimethylbutanoyl)-4-hydroxy-N—((R)-1-(4-(4-methylthiazol-5-yl)phenyl)-2-morpholinoethyl)pyrrolidine-2-carboxamide

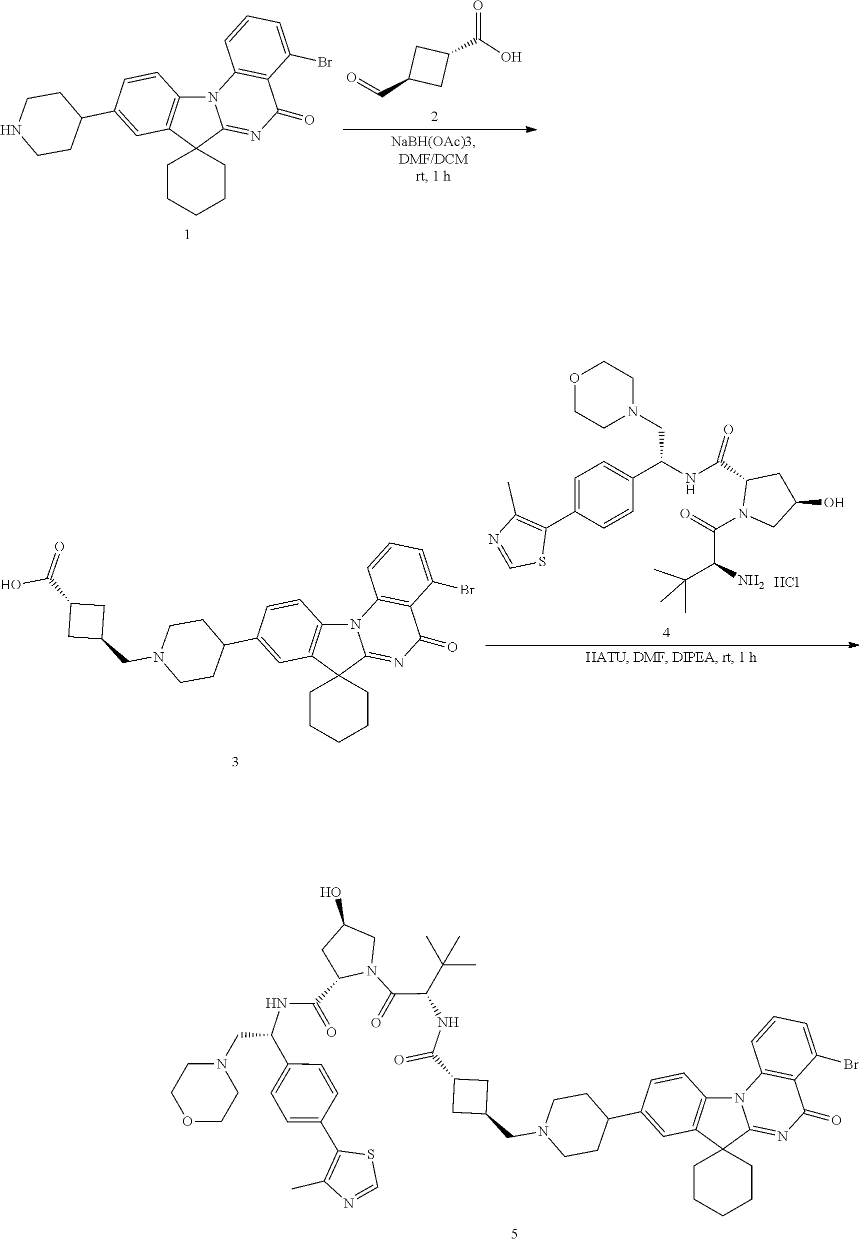

The synthetical procedure is similar to A¹¹. Target compound (8.0 mg) was obtained. ESI MS m/z: 1087.45 [M+H]⁺.

### Compound A22 (2S,4R)-1-((S)-2-(4-((4-(4′-bromo-5′-oxo-5′H-spiro[cyclohexane-1,7′-indolo[1,2-a]quinazolin]-9′-yl)piperidin-1-yl)methyl)bicyclo[2.2.1]heptane-1-carboxamido)-3,3-dimethylbutanoyl)-4-hydroxy-N—((R)-1-(4-(4-methylthiazol-5-yl)phenyl)-2-morpholinoethyl)pyrrolidine-2-carboramide

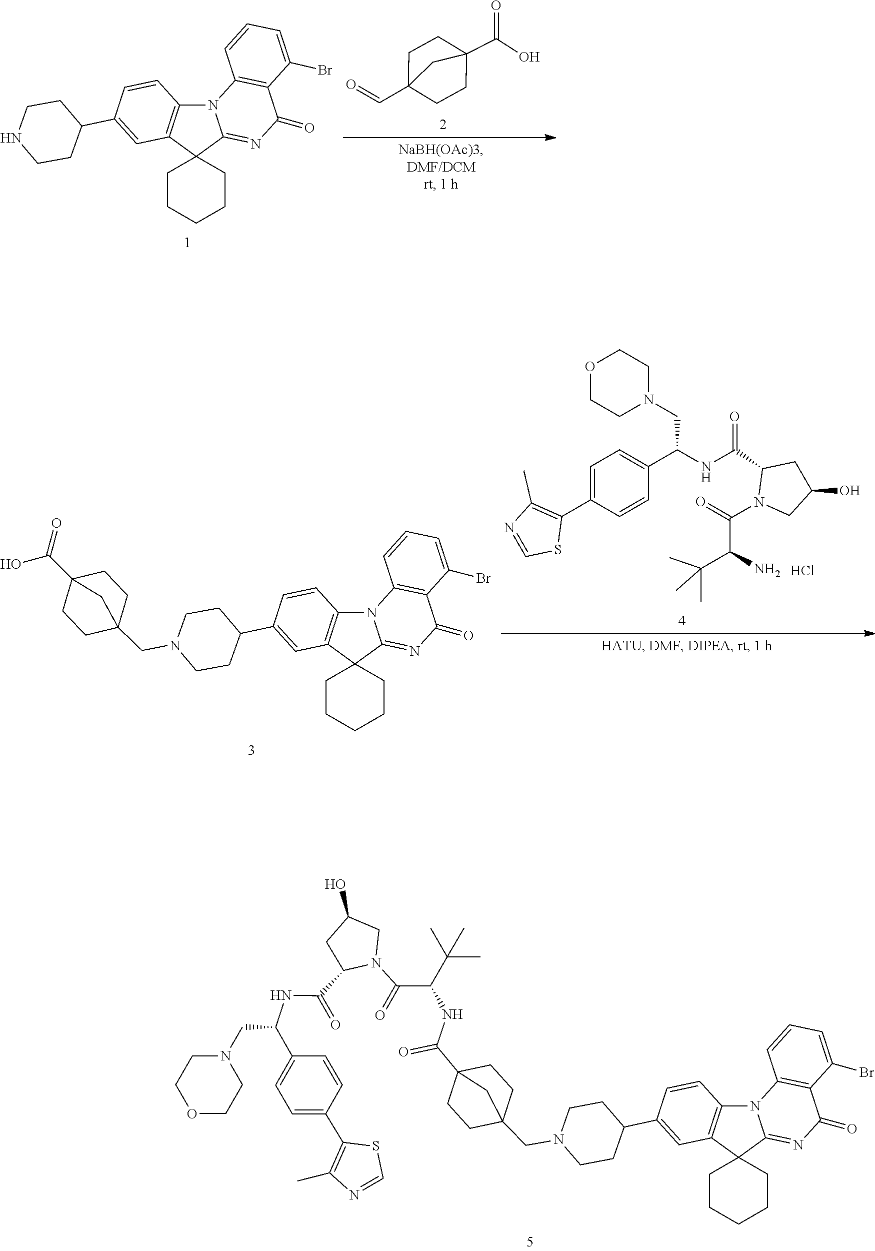

The synthetical procedure is similar to A¹¹. Target compound (7.3 mg) was obtained. ESI MS m/z: 1127.45 [M+H]⁺.

### Compound A23 (2S,4R)-1-((S)-2-(4-((4-(4′-bromo-5′-oxo-5′H-spiro[cyclohexane-1,7′-indolo[1,2-a]quinazolin]-10′-yl)piperidin-1-yl)methyl)bicyclo[2.2.1]heptane-1-carboxamido)-3,3-dimethylbutanoyl)-4-hydroxy-N—((S)-1-(4-(4-methylthiazol-5-yl)phenyl)ethyl)pyrrolidine-2-carboxamide

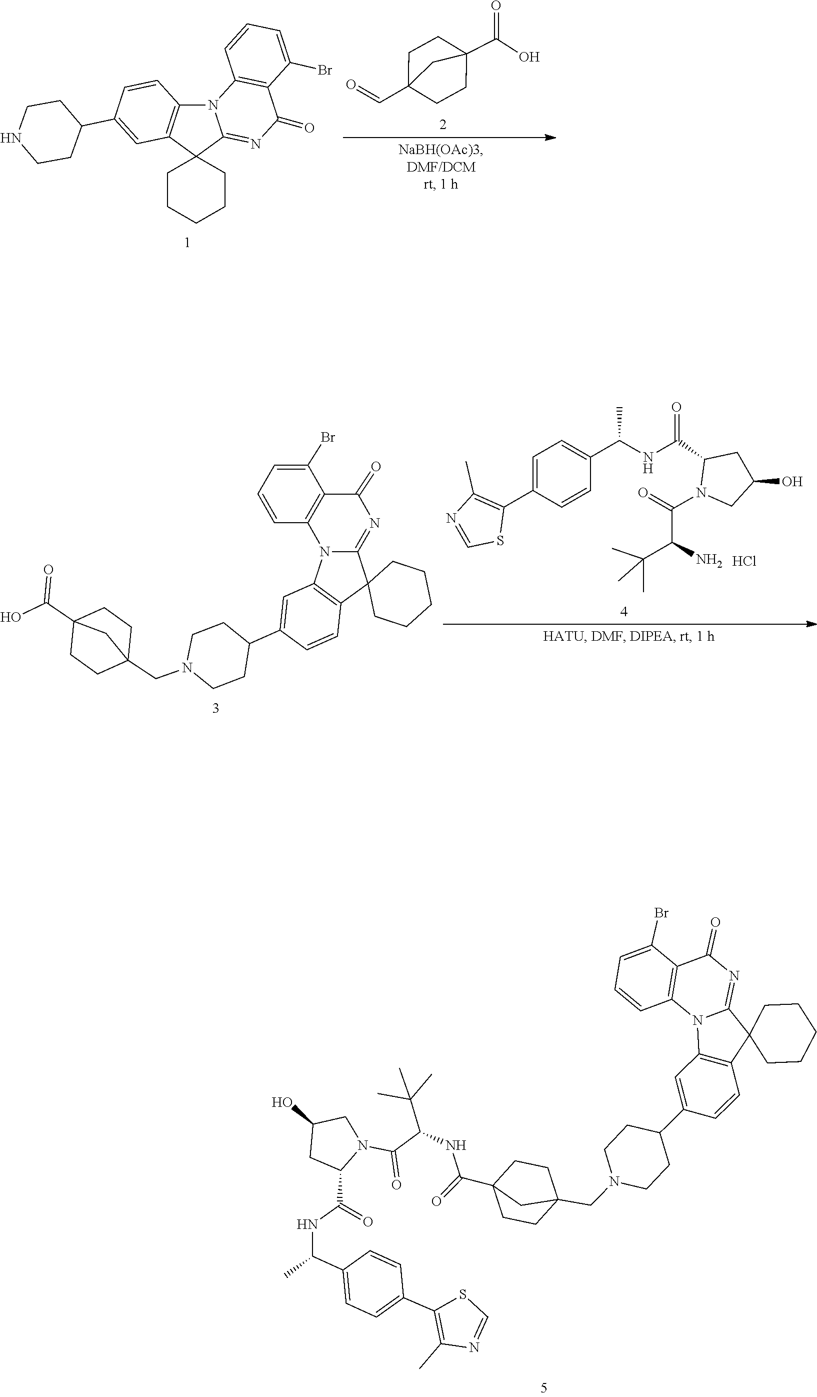

The synthetical procedure is similar to A¹¹. Target compound (6.2 mg) was obtained. ESI MS m/z: 1042.42 [M+H]⁺.

### Compound A24. (2S,4R)-1-((S)-2-(4-((4-(4′-bromo-5′-oxo-5′H-spiro[cyclohexane-1,7′-indolo[1,2-a]quinazolin]-10′-yl)piperidin-1-yl)methyl)bicyclo[2.2.1]heptane-1-carboxamido)-3,3-dimethylbutanoyl)-4-hydroxy-N—((R)-1-(4-(4-methylthiazol-5-yl)phenyl)-2-morpholinoethyl)pyrrolidine-2-carboxamide

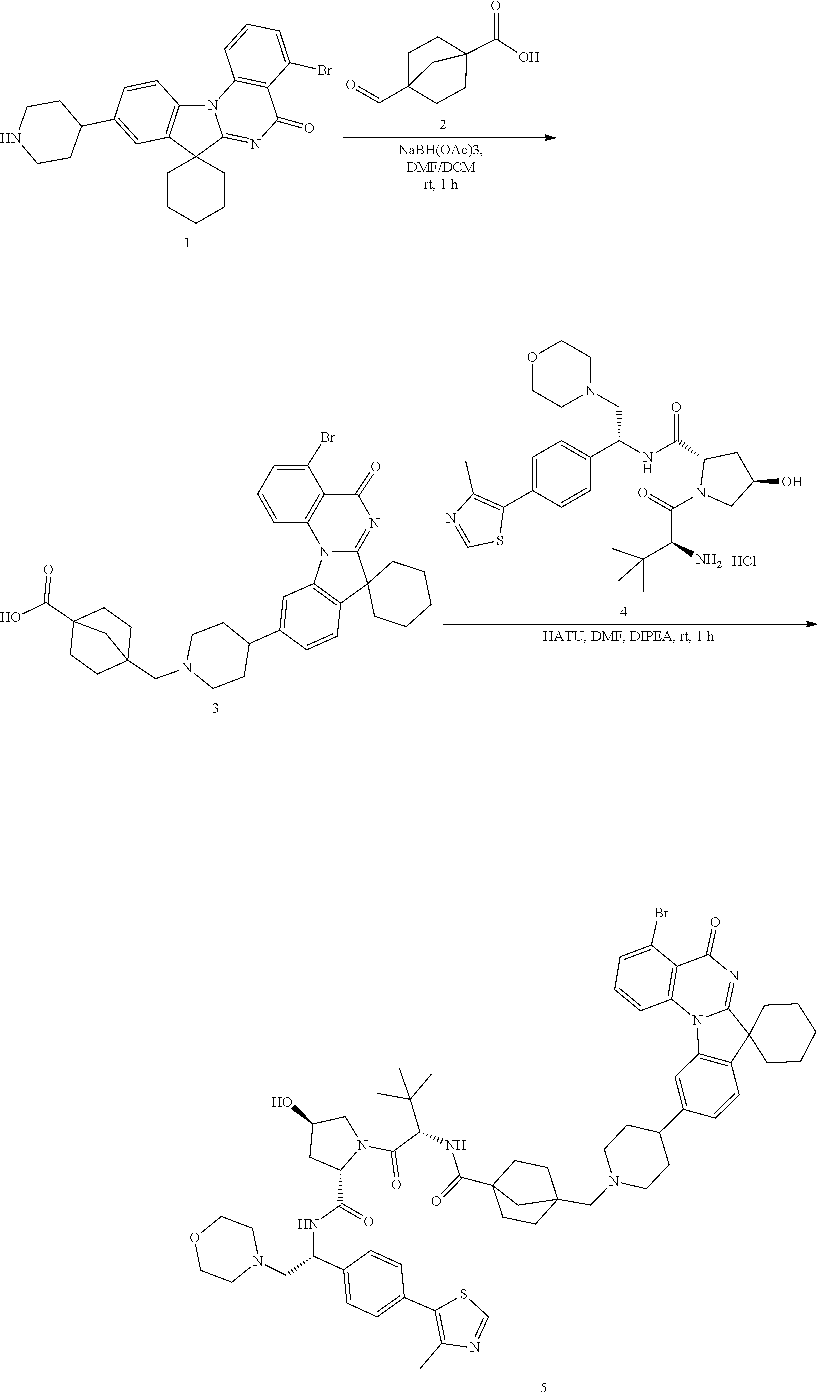

The synthetical procedure is similar to A¹¹. Target compound (8.3 mg) was obtained. ESI MS m/z: 1127.45 [M+H]⁺.

### Compound A25. (2S,4R)—N—((S)-6-(4-(4′-bromo-5′-oxo-5′H-spiro[cyclohexane-1,7-indolo[1,2-a]quinazolin]-9′-yl)piperidin-1-yl)-1-(4-chlorophenyl) hexyl)-1-((S)-2-(1-fluorocyclopropane-1-carboxamido)-3,3-dimethylbutanoyl)-4-hydroxypyrrolidine-2-carboxamide

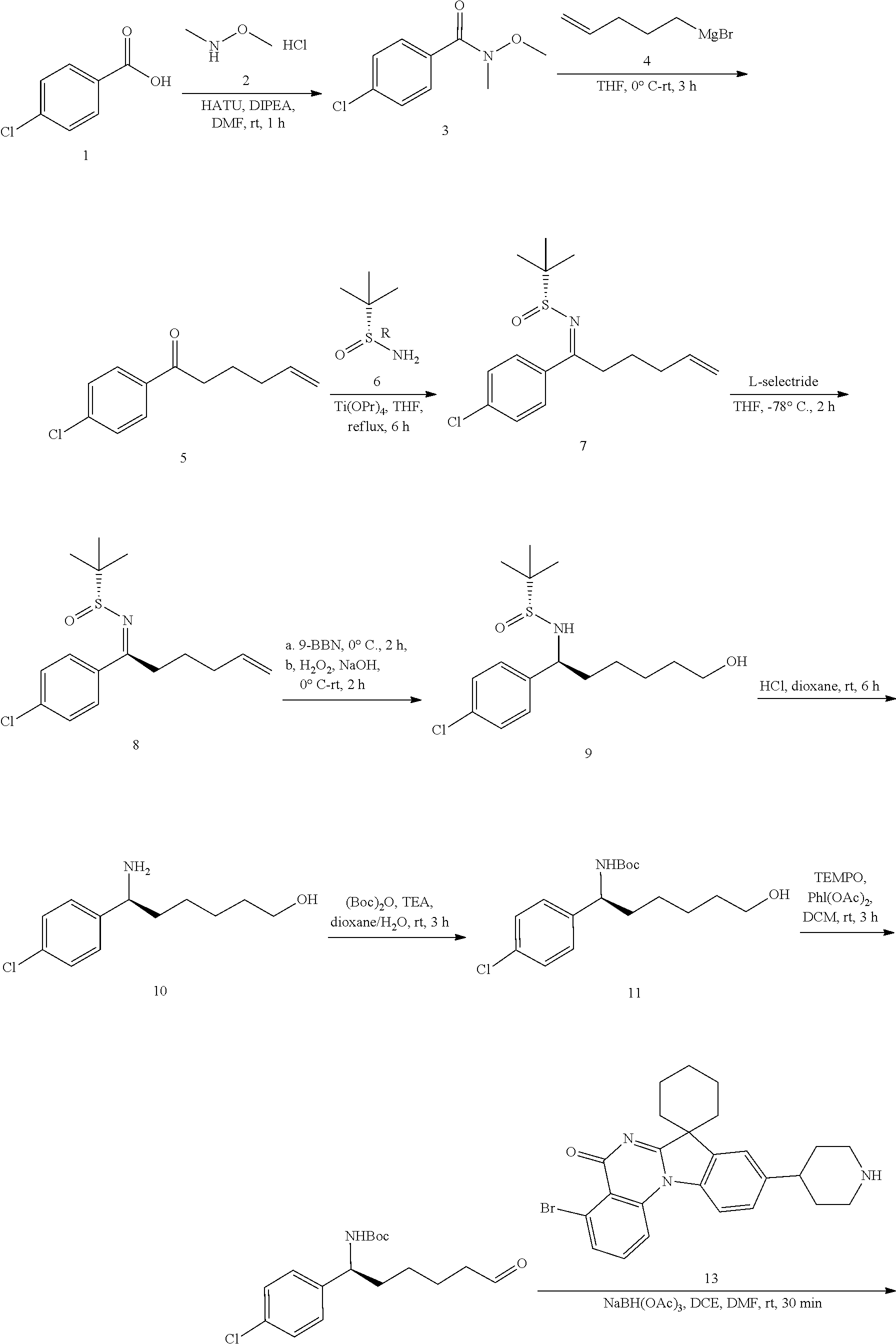

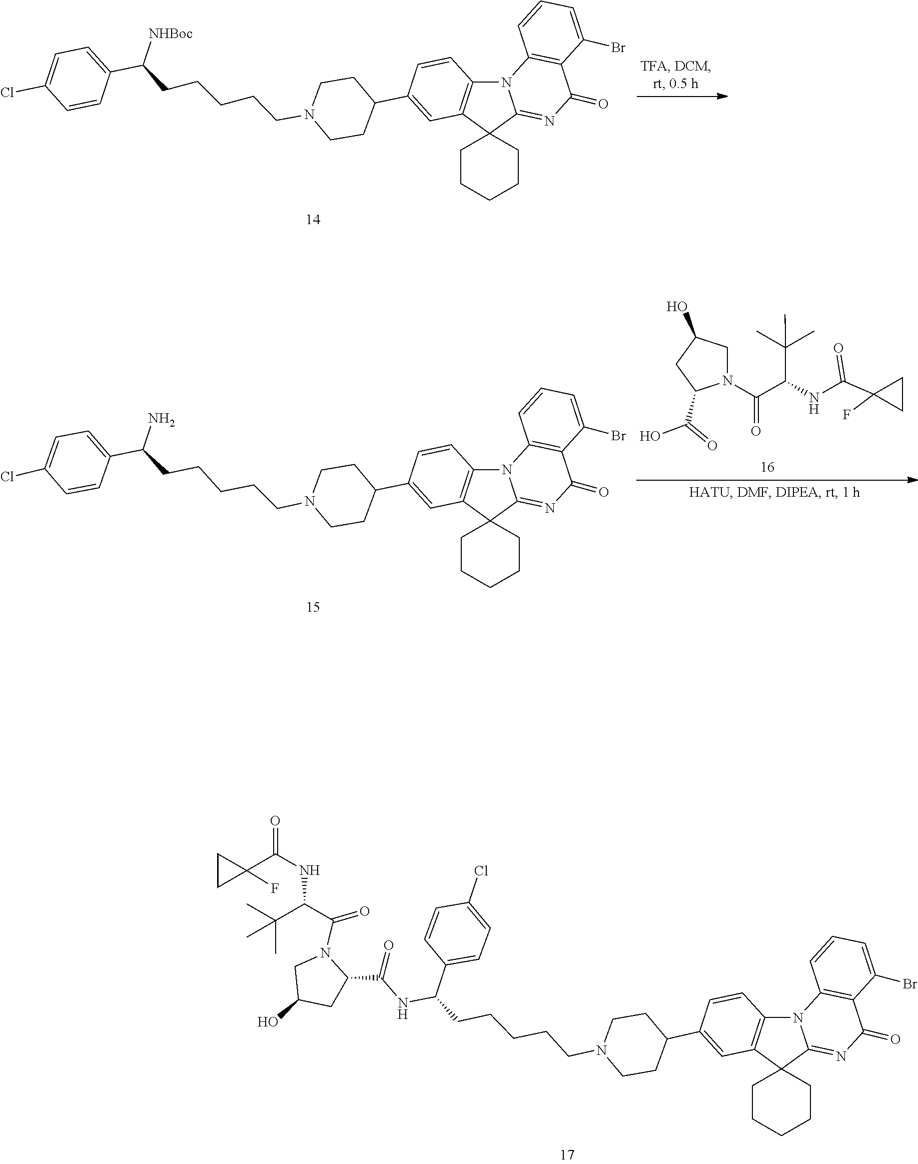

The synthetical procedure is similar to A². Target compound (7.8 mg) was obtained. ESI MS m/z: 985.37 [M+H]⁺.

### Compound A26. (2S,4R)—N—((S)-6-(4-(4′-bromo-5′-oxo-5′H-spiro[cyclohexane-1,7′-indolo[1,2-a]quinazolin]-10′-yl)piperidin-1-yl)-1-(4-chlorophenyl) hexyl)-1-((S)-2-(1-fluorocyclopropane-1-carboxamido)-3,3-dimethylbutanoyl)-4-hydroxypyrrolidine-2-carboxamide

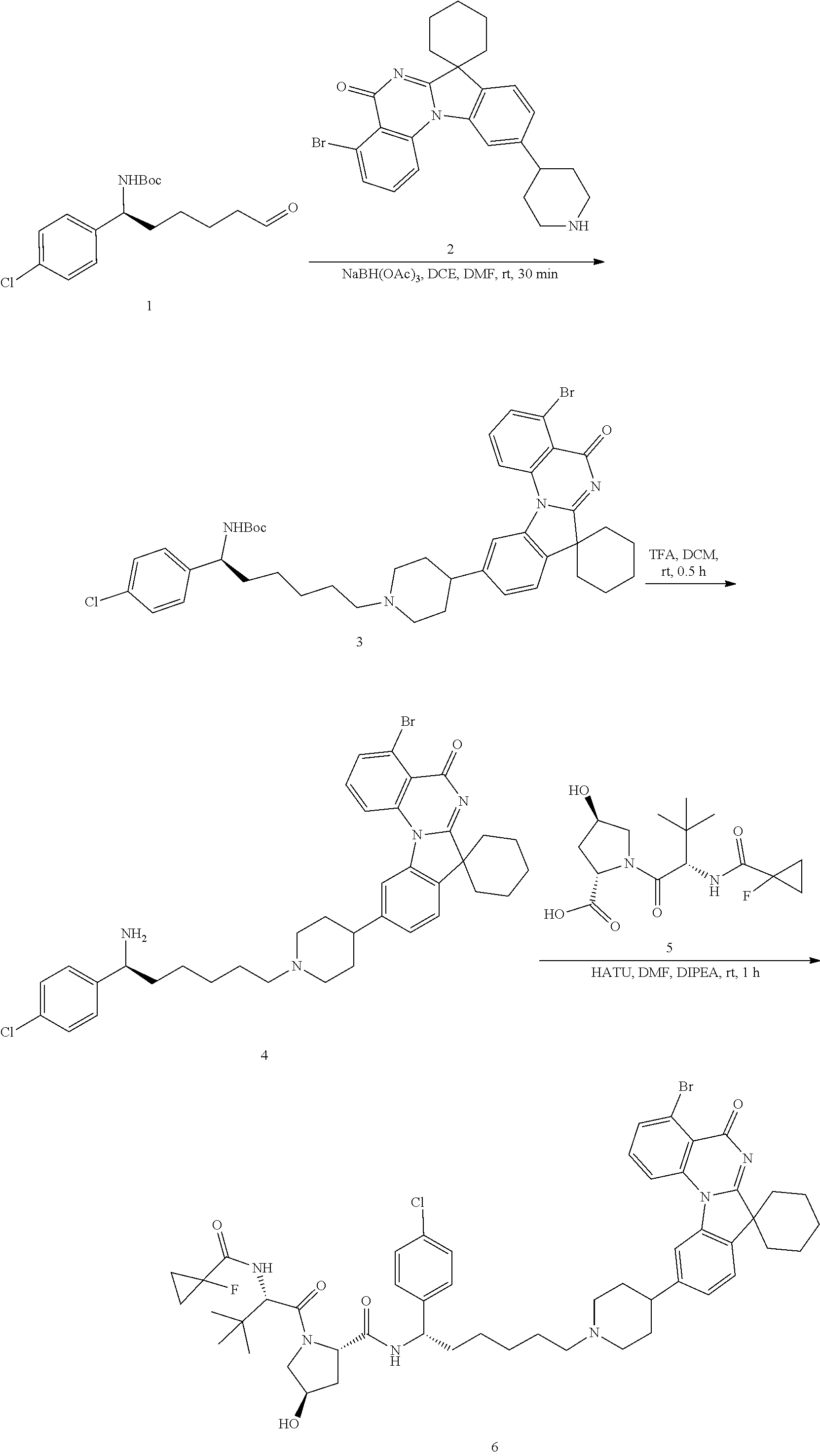 [SMILES: CC(C)(C)OC(=O)N[C@@H](CCCCC=O)c1ccc(Cl)cc1 ; O=c1nc2n(c3cccc(Br)c13)-c1cc(C3CCNCC3)ccc1C21CCCCC1 ; CC(C)(C)OC(=O)N[C@@H](CCCCCN1CCC(c2ccc3c(c2)-n2c(nc(=O)c4c(Br)cccc42)C32CCCCC2)CC1)c1ccc(Cl)cc1 ; N[C@@H](CCCCCN1CCC(c2ccc3c(c2)-n2c(nc(=O)c4c(Br)cccc42)C32CCCCC2)CC1)c1ccc(Cl)cc1 ; CC(C)(C)[C@H](NC(=O)C1(F)CC1)C(=O)N1C[C@H](O)C[C@H]1C(=O)O ; CC(C)(C)[C@H](NC(=O)C1(F)CC1)C(=O)N1C[C@H](O)C[C@H]1C(=O)N[C@@H](CCCCCN1CCC(c2ccc3c(c2)-n2c(nc(=O)c4c(Br)cccc42)C32CCCCC2)CC1)c1ccc(Cl)cc1]

The synthetical procedure is similar to A². Target compound (8.0 mg) was obtained. ESI MS m/z: 985.37 [M+H]⁺.

### Compound A27. (2S,4R)-1-((S)-2-(4-((4-(4′-bromo-5′-oxo-5′H-spiro[cyclohexane-1,7′-indolo[1,2-a]quinazolin]-9′-yl)piperidin-1-yl)methyl)bicyclo[2.2.2]octane-1-carboxamido)-3,3-dimethylbutanoyl)-4-hydroxy-N—((S)-1-(4-(4-methylthiazol-5-yl)phenyl)ethyl)pyrrolidine-2-carboxamide

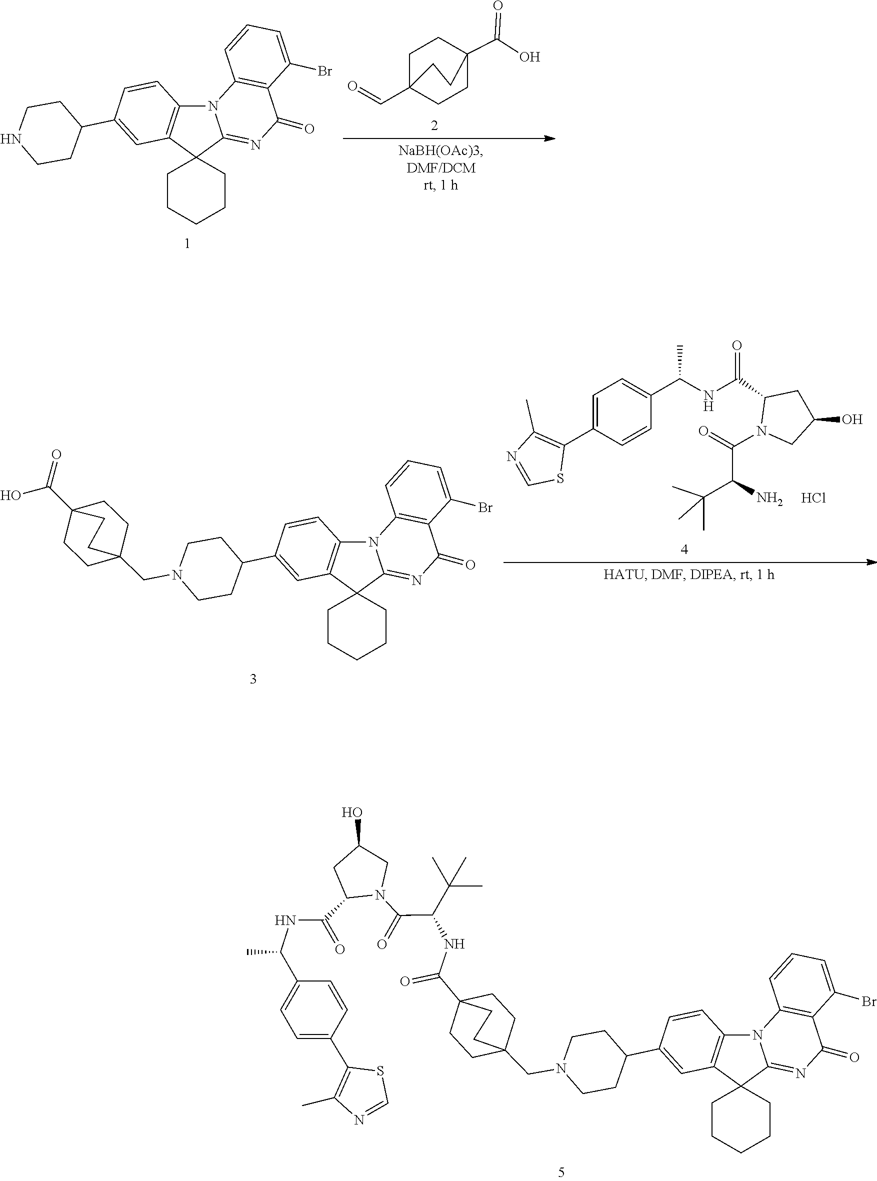

The synthetical procedure is similar to A¹¹. Target compound (5.7 mg) was obtained. ESI MS m/z: 1056.43 [M+H]⁺.

### Compound A28. (2S,4R)-1-((S)-2-(4-((4-(4′-bromo-5′-oxo-5′H-spiro[cyclohexane-1,7′-indolo[1,2-a]quinazolin]-9′-yl)piperidin-1-yl)methyl)bicyclo[2.2.2]octane-1-carboxamido)-3,3-dimethylbutanoyl)-4-hydroxy-N—((R)-1-(4-(4-methylthiazol-5-yl)phenyl)-2-morpholinoethyl)pyrrolidine-2-carboxamide

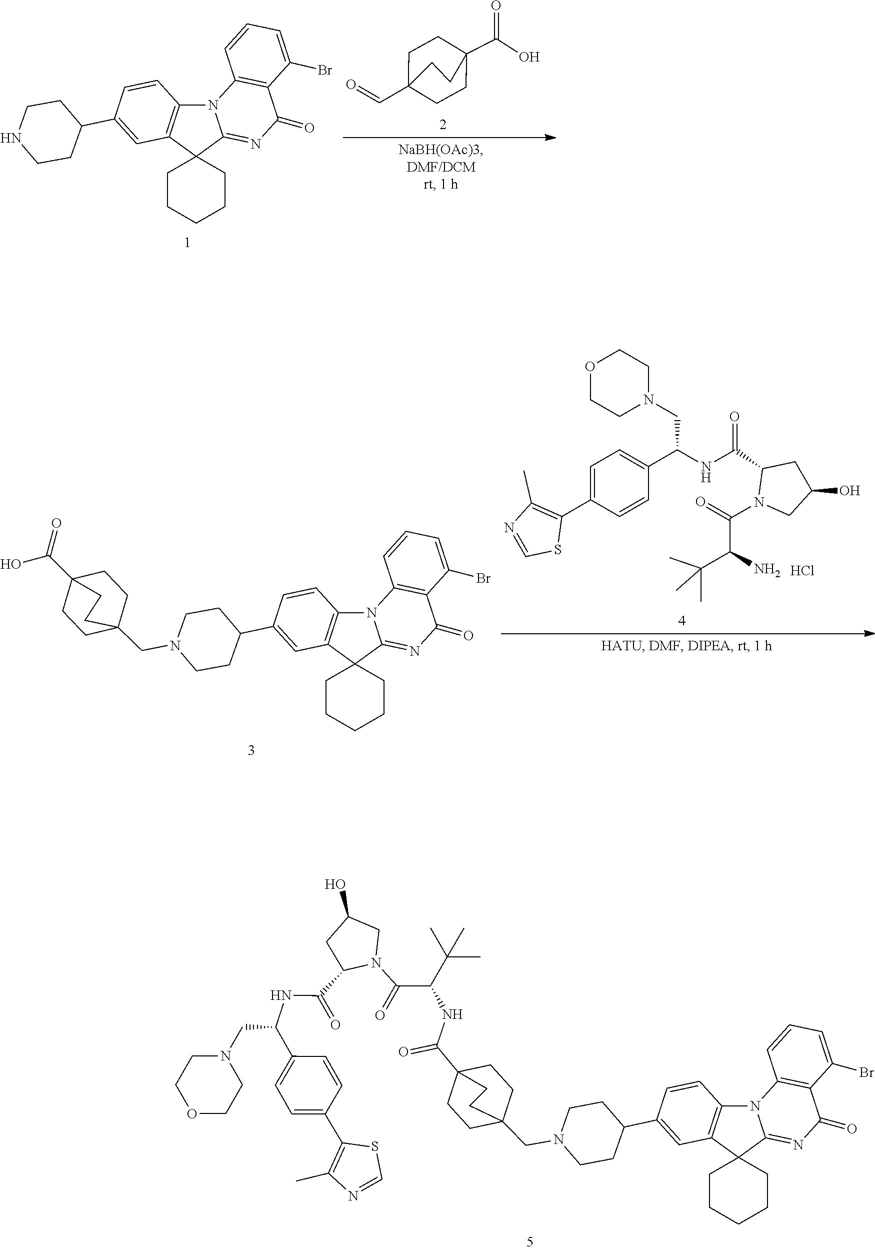

The synthetical procedure is similar to A¹¹. Target compound (6.2 mg) was obtained. ESI MS m/z: 1141.48 [M+H]⁺.

### Compound A29. (2S,4R)-1-(S)-2-(4-(4-(4′-bromo-5′-oxo-5′H-spiro[cyclohexane-1,7′-indolo[1,2-a]quinazolin]-10′-yl)piperidin-1-yl)methyl)bicyclo[2.2.2]octane-1-carboxamido)-3,3-dimethylbutanoyl)-4-hydroxy-N—(S)-1-(4-(4-methylthiazol-5-yl)phenyl)ethyl)pyrrolidine-2-carboxamide

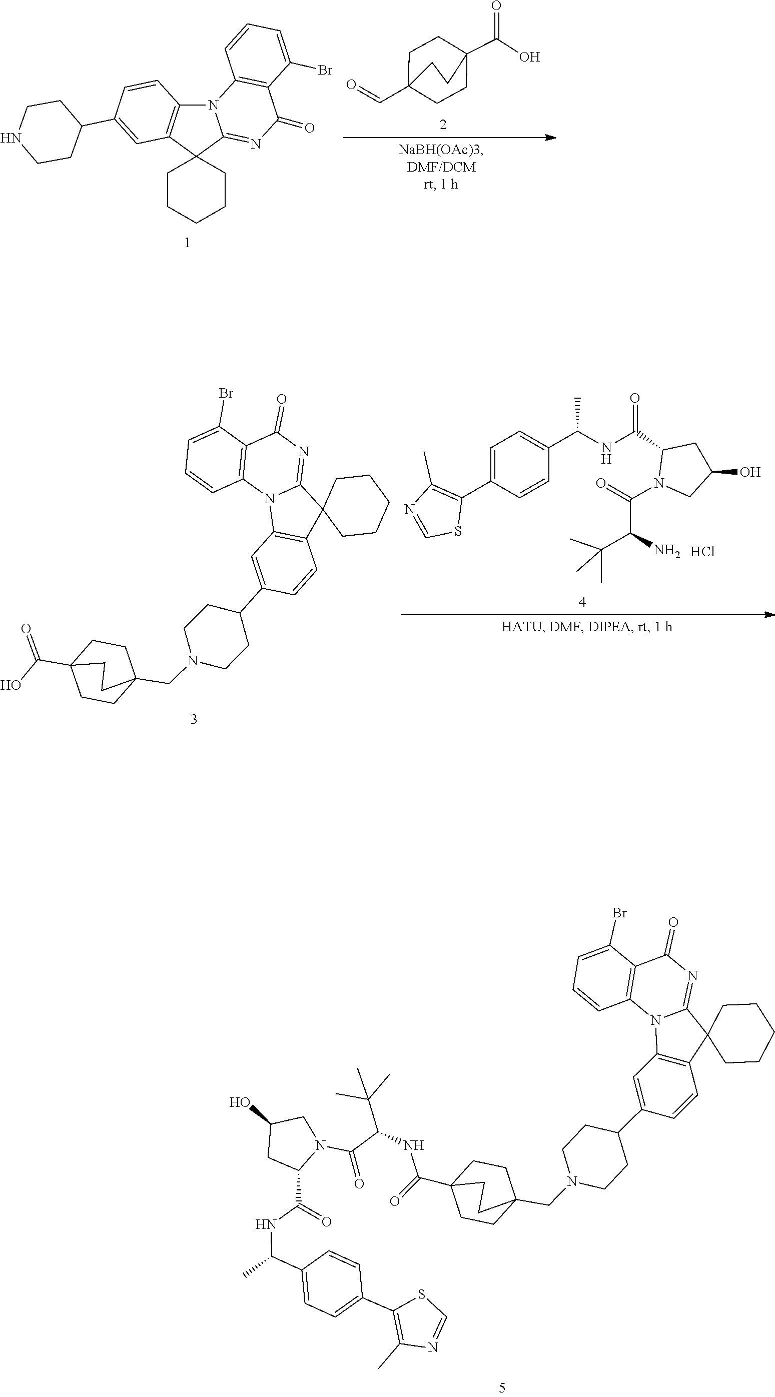

The synthetical procedure is similar to A¹¹. Target compound (6.2 mg) was obtained. ESI MS m/z: 1056.43 [M+H]⁺.

### Compound A30. (2S,4R)-1-((S)-2-(4-((4-(4′-bromo-5′-oxo-5′H-spiro[cyclohexane-1,7′-indolo[1,2-a]quinazolin]-10′-yl)piperidin-1-yl)methyl)bicyclo[2.2.2]octane-1-carboxamido)-3,3-dimethylbutanoyl)-4-hydroxy-N—((R)-1-(4-(4-methylthiazol-5-yl)phenyl)-2-morpholinoethyl)pyrrolidine-2-carboxamide

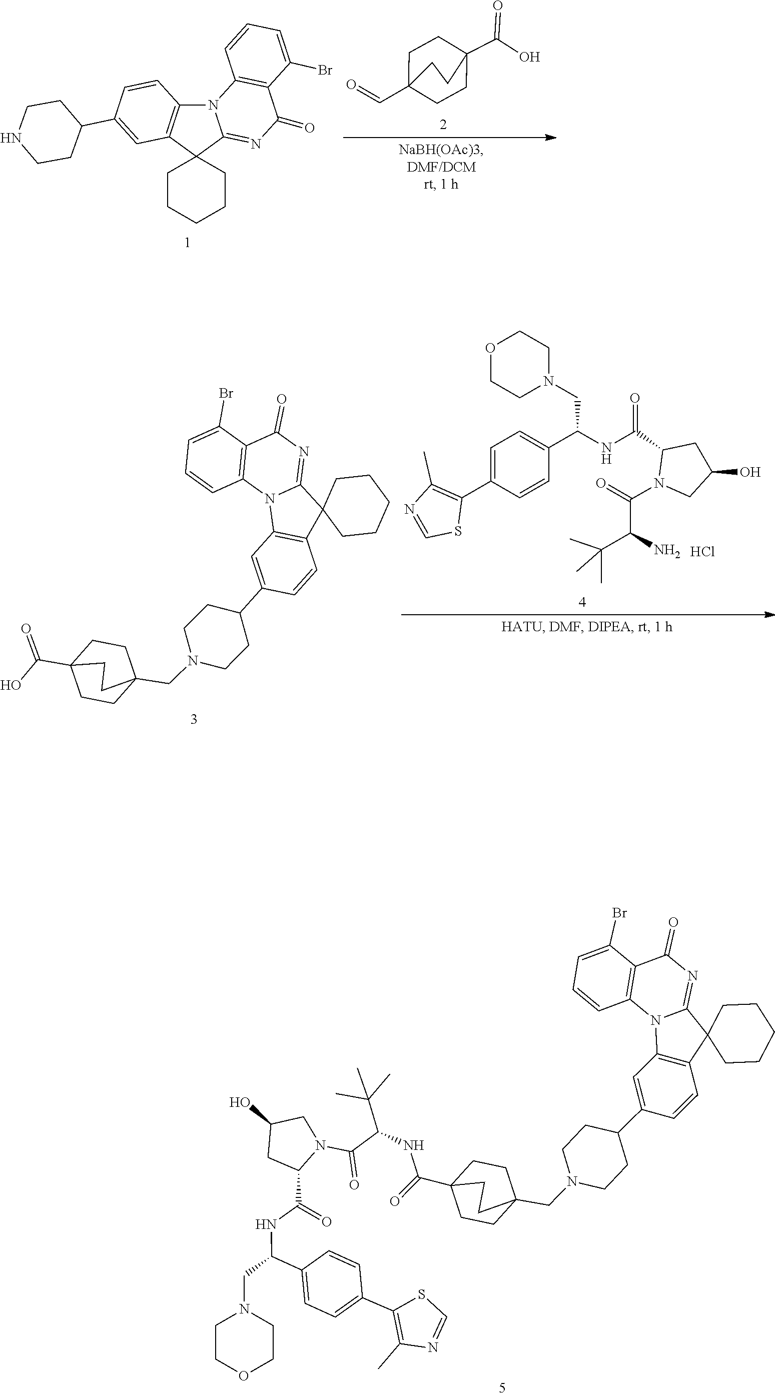

The synthetical procedure is similar to A¹¹. Target compound (3.6 mg) was obtained. ESI MS m/z: 1141.48 [M+H]⁺.

### Compound A31. (2S,4R)-1-((S)-2-(4-((1r,4S)-4-((4-(4′-bromo-5′-oxo-5′H-spiro[cyclohexane-1,7′-pyrimido[5′,4′: 4,5]pyrrolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

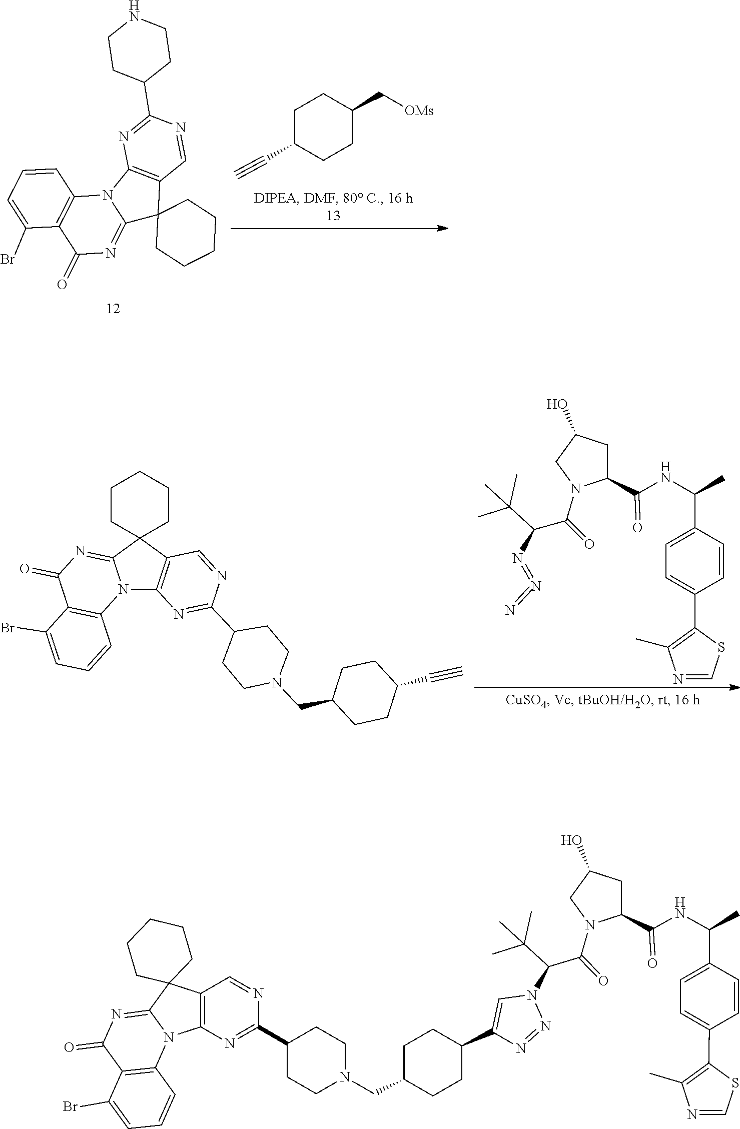

The synthetical procedure is similar to former procedure. Target compound (5.3 mg) was obtained. ESI MS m/z: 1056.42 [M+H]⁺.

### Compound A32. (2S,4R)-1-((S)-2-(4-((1r,4S)-4-((4-(4′-bromo-5′-oxo-5′H-spiro[cyclohexane-1,7′-pyrimido[5′,4′: 4,5]pyrrolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-1-(4-(4-methylthiazol-5-yl)phenyl)-2-morpholinoethyl)pyrrolidine-2-carboxamide

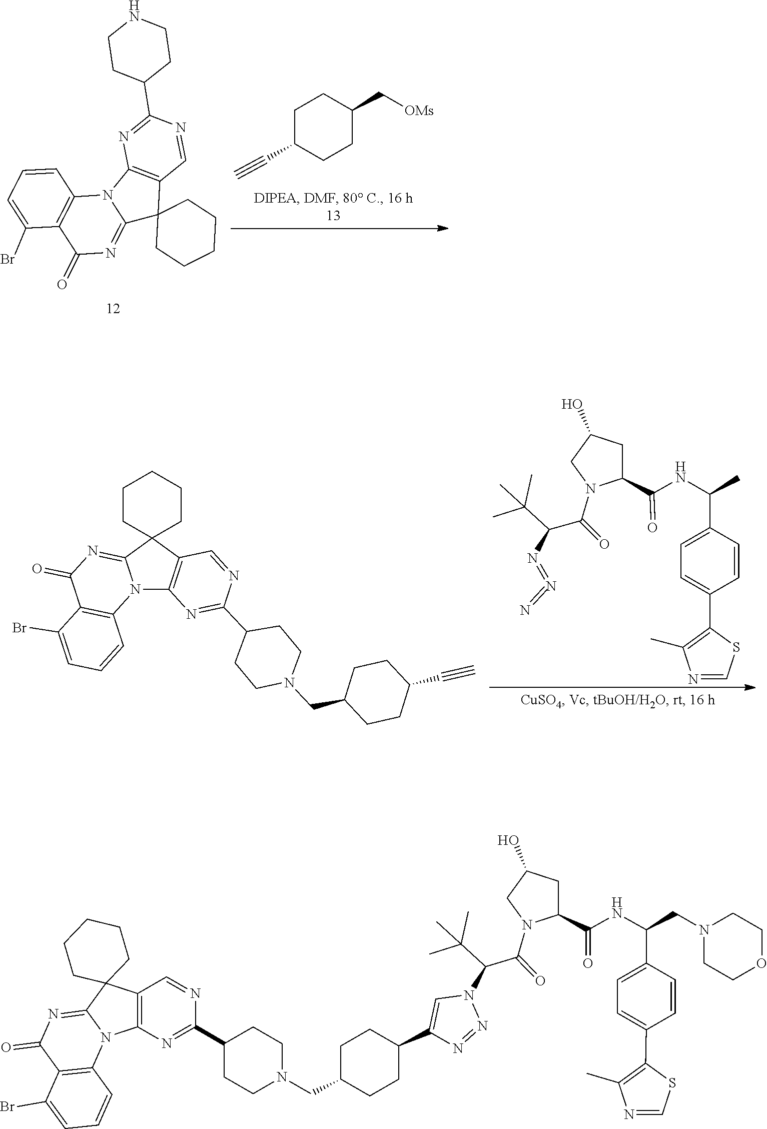

The synthetical procedure is similar to former procedure. Target compound (6.2 mg) was obtained. ESI MS m/z: 1141.47 [M+H]⁺.

### Compound A33. (2S,4R)—N—((R)-2-(4-((1r,4R)-4-((4-(4′-bromo-5′-010-5′H-spiro[cyclohexane-1,7′-indolo[1,2-a]quinazolin]-9′-yl)piperidin-1-yl)methyl)cyclohexyl)-1H-1,2,3-triazol-1-yl)-1-(4-(4-methylthiazol-5-yl)phenyl)ethyl)-1-((S)-2-(1-fluorocyclopropane-1-carboxamido)-3,3-dimethylbutanoyl)-4-hydroxypyrrolidine-2-carboxamide

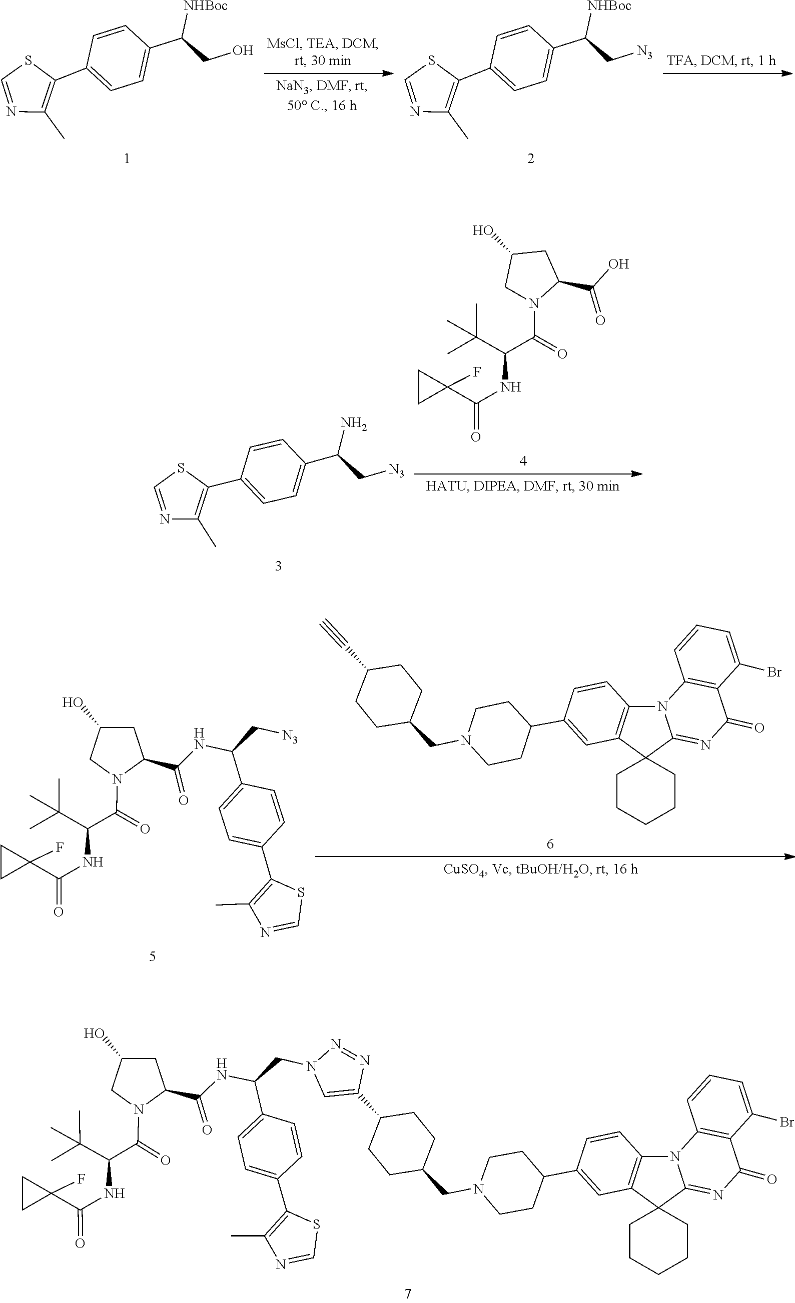

Synthesis of 2, To a solution of 1 (500 mg, 1.5 mmol, 1 equiv.) in DCM (30 mL), was added TEA (300 mg, 3 mmol, 2 equiv.) and then MsCl (205 mg, 1.80 mmol, 1.2 equiv.) at room temperature. The mixture was stirred 15 min. After reaction, then a saturated NaHCO₃ solution was add and the mixture was stirred for 10 minutes. The DCM phase was collected and dried by anhydrous Na₂SO₄. After removing solvent, the residue was dissolved into DMF (10 mL), NaN₃ (194 mg, 3.0 mmol, 2 equiv.) was added. The mixture was stirred for 16 h at 50° C. Water (30 mL) was added to quench the reaction and product was extracted with ethyl acetate twice (30 mL×2). Combined organic phases were washed by brine twice, dried by anhydrous Na₂SO₄. After removing solvent, the residue was purified by silica gel column chromatography to get target compound. (elution solvents: ethyl acetate/hexane (1/1, v/v) to get pure product 2 (410 mg, 76%). ESI MS m/z: 360.14 [M+H]⁺.

Synthesis of 5, To a solution of 2 (400 mg, 1.11 mmol, 1 eq) in DCM (10 mL) was added TFA (5 mL), the mixture was stirred for 30 minutes. After removing solvent and TFA, the residue was redissolved into DMF (10 mL), 4 (368 mg, 3.33 mmol, 1 eq.), HATU (637 mg, 1.67 mmol, 1.5 eq) and TEA (430 mg, 3.33 mmol, 3 eq) were added. The mixture was stirred for 30 minutes. The solution was purified by C-18 reversal column to get target compound 5 (430 mg, 68%) ESI MS m/z: 572.24 [M+H]⁺.

Synthesis of 7, this procedure is similar to former procedure. Target product was received. ESI MS m/z: 1155.46 [M+H]⁺.

### Compound A34. (2S,4R)—N—((R)-2-(4-((1r,4R)-4-((4-(4′-bromo-5′-oxo-5′H-spiro[cyclohexane-1,7′-pyrido[3′,2′: 4,5]pyrrolo[1,2-a]quinazolin]-10′-yl)piperidin-1-yl)methyl)cyclohexyl)-1H-1,2,3-triazol-1-yl)-1-(4-(4-methylthiazol-5-yl)phenyl)ethyl)-1-((S)-2-(1-fluorocyclopropane-1-carboxamido)-3,3-dimethylbutanoyl)-4-hydroxypyrrolidine-2-carboxamide

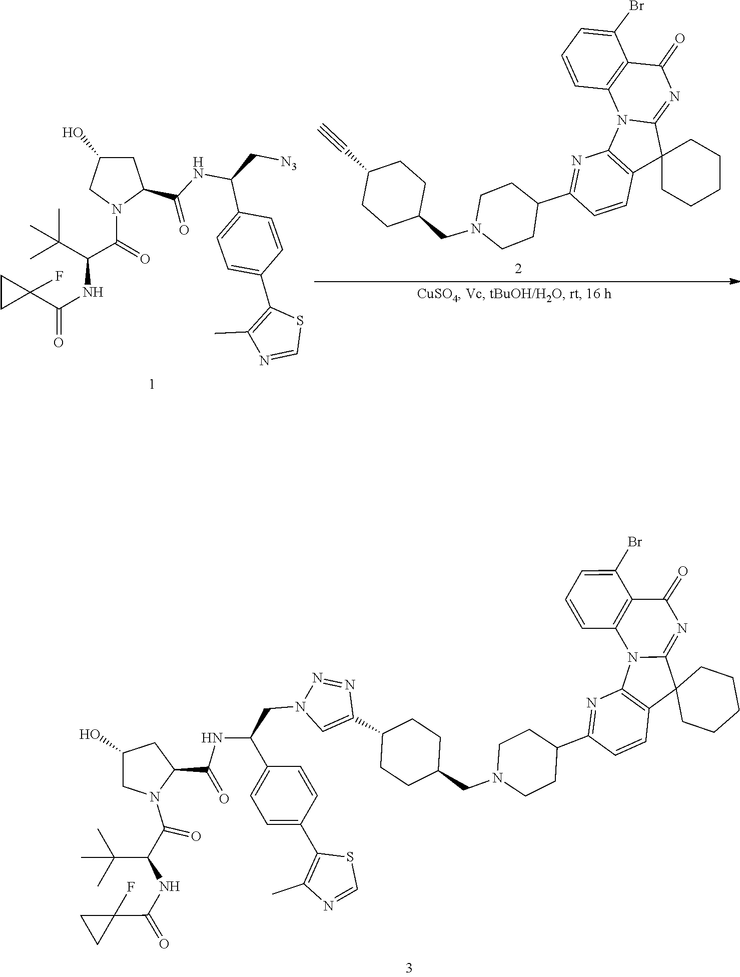 [SMILES: Cc1ncsc1-c1ccc([C@H](CN=[N+]=[N-])NC(=O)[C@@H]2C[C@@H](O)CN2C(=O)[C@@H](NC(=O)C2(F)CC2)C(C)(C)C)cc1 ; C#C[C@H]1CC[C@H](CN2CCC(c3ccc4c(n3)-n3c(nc(=O)c5c(Br)cccc53)C43CCCCC3)CC2)CC1 ; Cc1ncsc1-c1ccc([C@H](Cn2cc([C@H]3CC[C@H](CN4CCC(c5ccc6c(n5)-n5c(nc(=O)c7c(Br)cccc75)C65CCCCC5)CC4)CC3)nn2)NC(=O)[C@@H]2C[C@@H](O)CN2C(=O)[C@@H](NC(=O)C2(F)CC2)C(C)(C)C)cc1]

The synthetical procedure is similar to former procedure. Target compound (5.2 mg) was obtained. ESI MS m/z: 1056.45 [M+H]⁺.

### Compound A35. (2R,4S)—N—((S)-6-(4-(4′-bromo-5′-oxo-5′H-spiro[cyclohexane-1,7′-indolo[1,2-a]quinazolin]-9′-yl)piperidin-1-yl)-1-(4-(4-methylthiazol-5-yl)phenyl) hexyl)-1-((S)-2-(4-cyclopropyl-1H-1,2,3-triazol-1-yl)-3,3-dimethylbutanoyl)-4-hydroxypyrrolidine-2-carboxamide

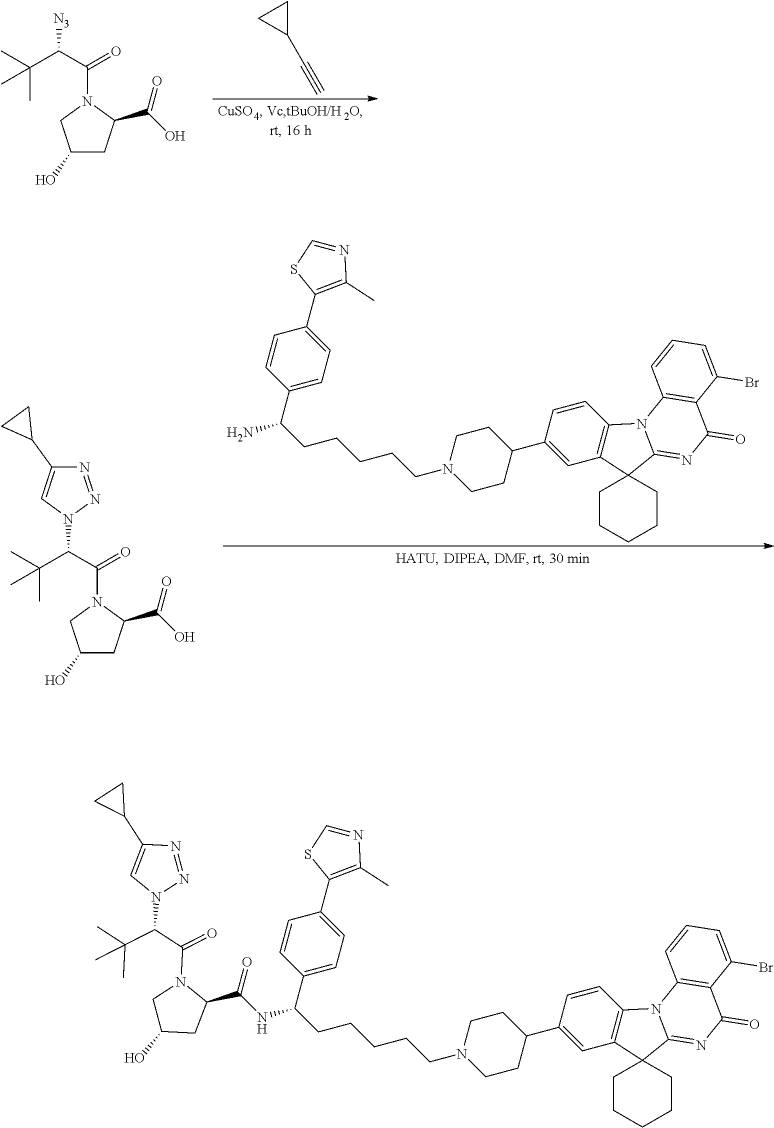

The synthetical procedure is similar to former procedure. Target compound (4.6 mg) was obtained. ESI MS m/z: 1054.43 [M+H]⁺.

### Compound A36. (2R,4S)—N—((S)-6-(4-(4′-bromo-5′-oxo-5′H-spiro[cyclohexane-1,7′-indolo[1,2-a]quinazolin]-10′-yl)piperidin-1-yl)-1-(4-(4-methylthiazol-5-yl)phenyl) hexyl)-1-((S)-2-(4-cyclopropyl-1H-1,2,3-triazol-1-yl)-3,3-dimethylbutanoyl)-4-hydroxypyrrolidine-2-carboxamide

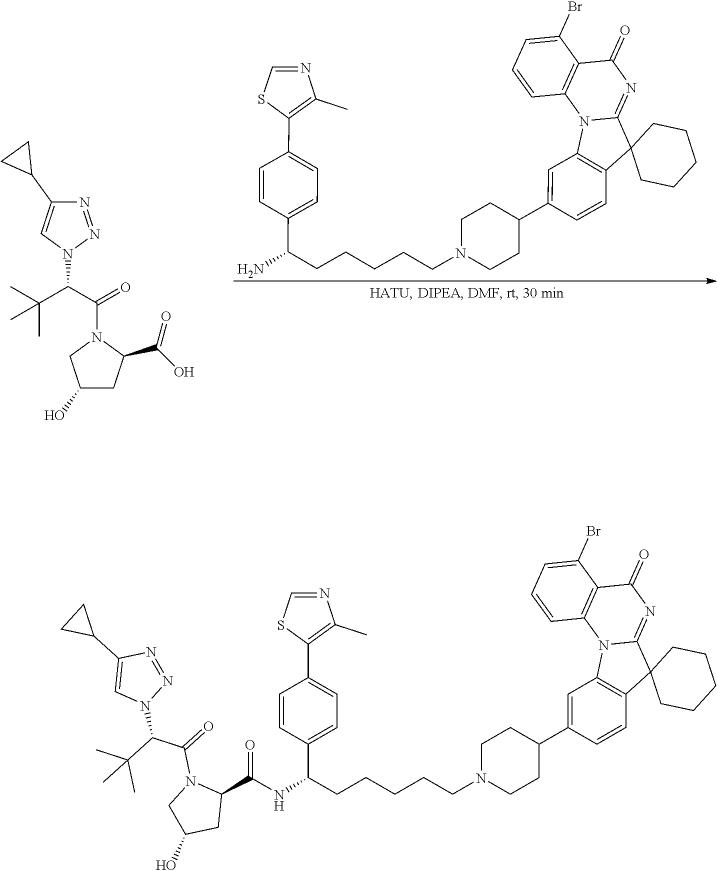

The synthetical procedure is similar to former procedure. Target compound (6.2 mg) was obtained. ESI MS m/z: 1054.43 [M+H]⁺.

### Compound A37. (2R,4S)—N—((S)-6-(4-(4′-bromo-5′-oxo-5′H-spiro[cyclohexane-1,7′-pyrido[3′,2′: 4,5]pyrrolo[1,2-a]quinazolin]-10′-yl)piperidin-1-yl)-1-(4-(4-methylthiazol-5-yl)phenyl) hexyl)-1-((S)-2-(4-cyclopropyl-1H-1,2,3-triazol-1-yl)-3,3-dimethylbutanoyl)-4-hydroxypyrrolidine-2-carboxamide

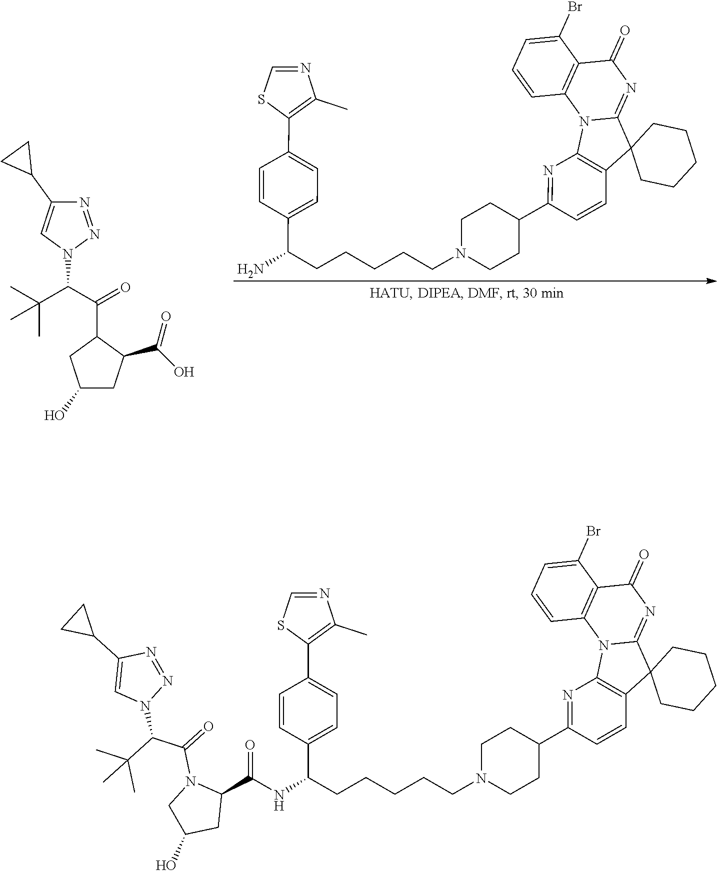

The synthetical procedure is similar to former procedure. Target compound (3.5 mg) was obtained. ESI MS m/z: 1055.42 [M+H]⁺.

### Compound A38. (2R,4S)—N—((S)-6-(4-(4′-bromo-5′-oxo-5′H-spiro[cyclohexane-1,7′-indolo[1,2-a]quinazolin]-9′-yl)piperidin-1-yl)-1-(4-(4-methylthiazol-5-yl)phenyl) hexyl)-1-((S)-3,3-dimethyl-2-(1-(morpholinomethyl)cyclopropane-1-carboxamido) butanoyl)-4-hydroxypyrrolidine-2-carboxamide

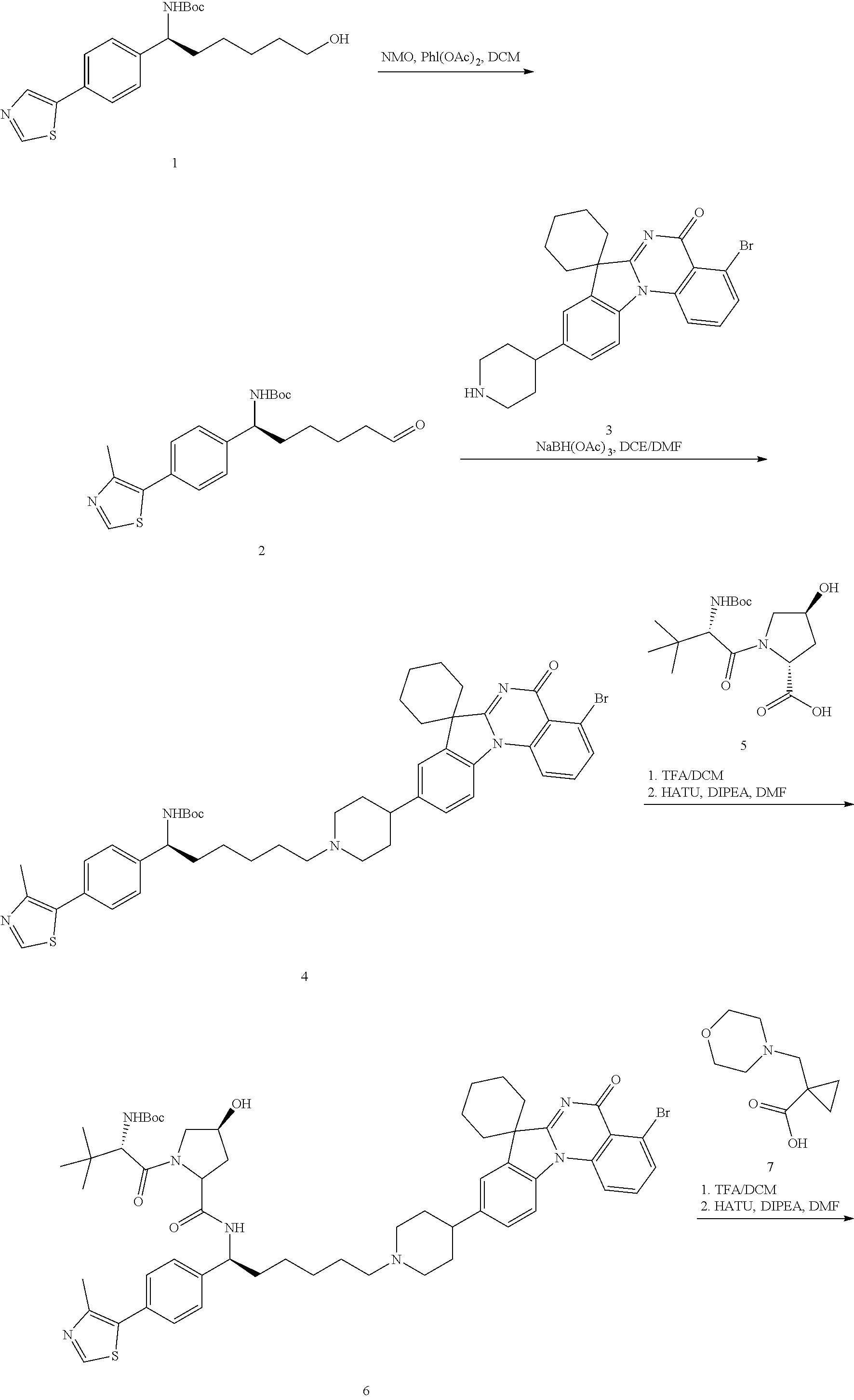

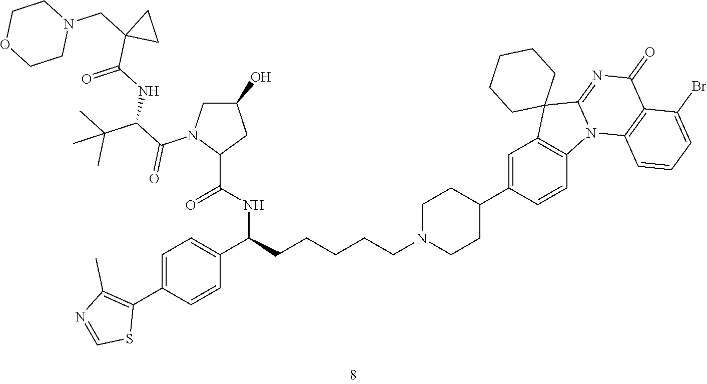

Compound A38 was synthesized using the similar procedures as used in the synthesis of Compound A17.

Calcd. (M+H)⁺ m/z: 1129.5, Found (M+H)⁺ m/z: 1129.7

### Compound A39. (2S,4R)-1-((S)-2-(2-(4-(4′-chloro-5′-oxo-5′H-spiro[cyclohexane-1,7′-indolo[1,2-a]quinazolin]-10′-yl)piperidin-1-yl) acetamido)-3,3-dimethylbutanoyl)-4-hydroxy-N—((S)-1-(4-(4-methylthiazol-5-yl)phenyl)ethyl)pyrrolidine-2-carboxamide

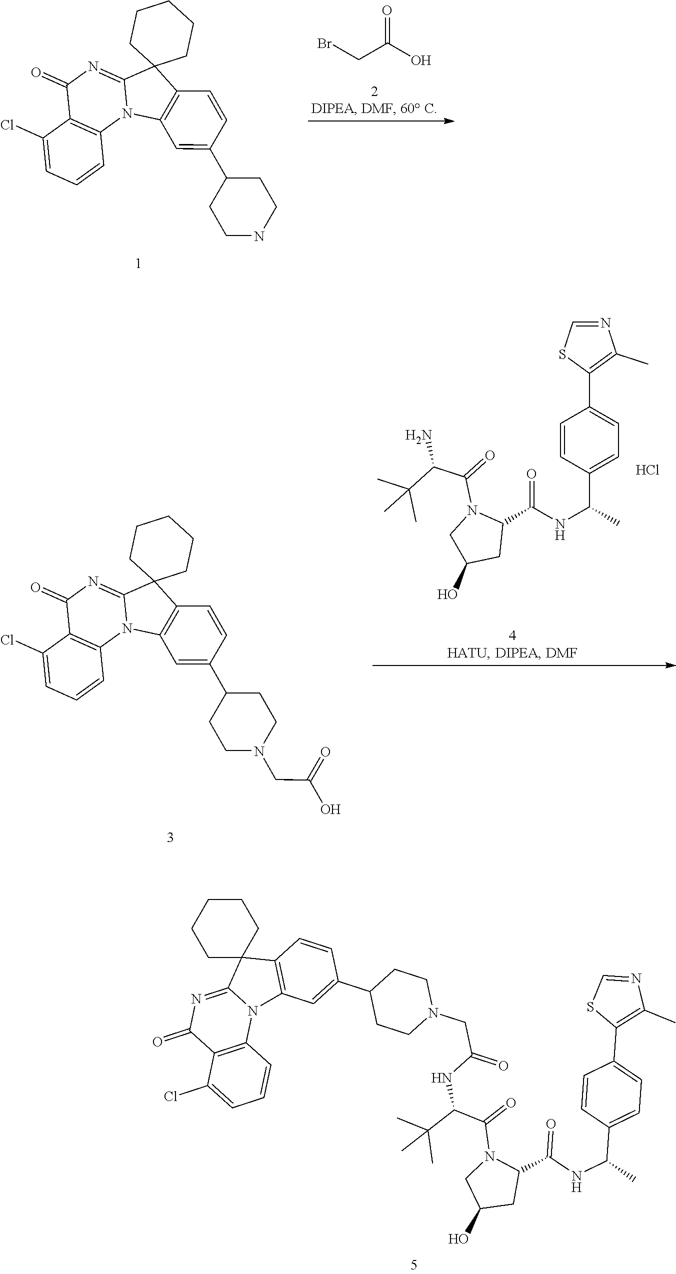

Step 1: To a stirred solution of compound 1 (7.3 mg 0.014 mmol, 1.0 eq) in DMF (0.5 mL) was added bromoacetic acid (1.9 mg, 0.014 mmol, 1.0 eq) DIPEA (0.020 mL, 0.14 mmol, 10 eq). The reaction mixture was stirred at 60° C. for 2 hours. The mixture was subjected to reverse phase flash chromatography (10-100% MeCN in H₂O, 0.1% TFA) to give compound 3 (3.1 mg, 37%).

Step 2: To a stirred solution of compound 3 (3.1 mg, 0.0052 mmol, 1.0 eq), compound 4 (2.5 mg, 0.0052 mg, 1.0 eq) and HATU (2.2 mg, 0.0058 mmol, 1.1 eq) was added DIPEA (0.008 mL, 0.052 mmol, 10 eq) and the reaction mixture was stirred for 30 minutes. The mixture was subjected to Prep-HPLC (30-100% MeCN in H₂O, 0.1% TFA) to give compound 5 (2.2 mg, 47%).

Calcd. (M+H)⁺ m/z: 904.4, Found (M+H)⁺ m/z: 904.5

### Compound A40. (2S,4R)-1-((S)-2-(2-(4-(4′-bromo-5′-oxo-5′H-spiro[cyclohexane-1,7′-indolo[1,2-a]quinazolin]-10′-yl)piperidin-1-yl) acetamido)-3,3-dimethylbutanoyl)-4-hydroxy-N—((S)-1-(4-(4-methylthiazol-5-yl)phenyl)ethyl)pyrrolidine-2-carboxamide

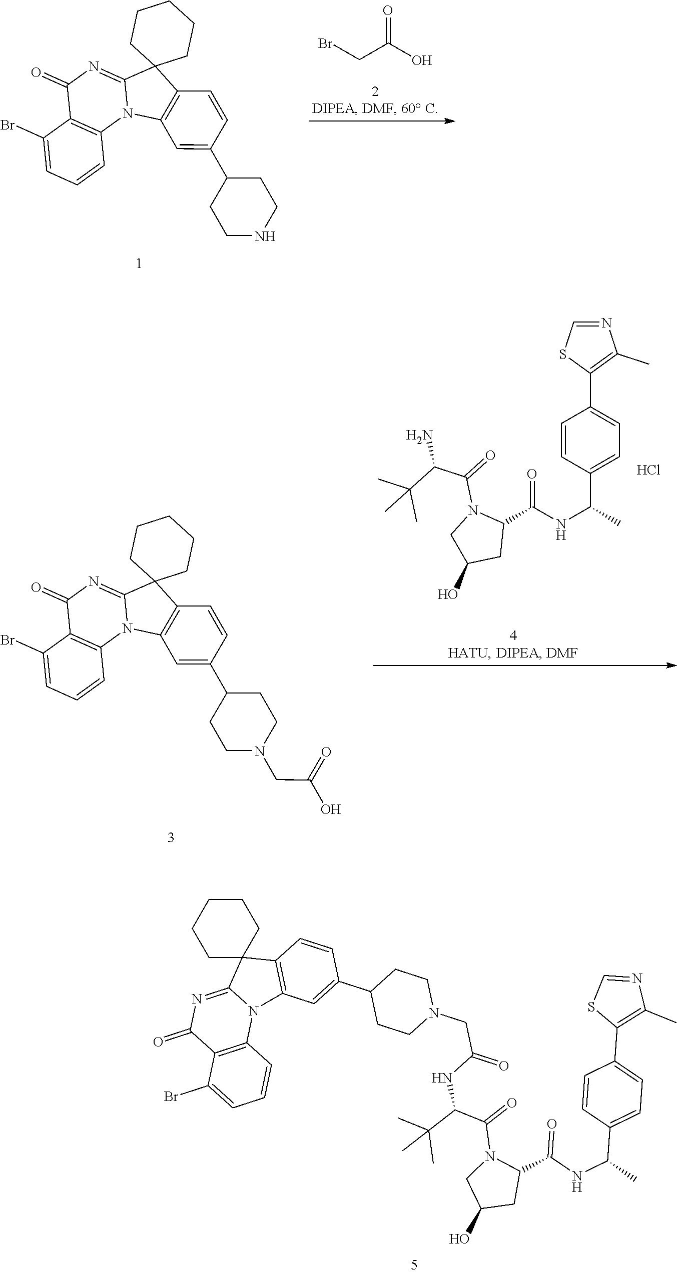

Compound A40 was synthesized using the similar procedures as used in the synthesis of Compound A39.

Calcd. (M+H)⁺ m/z: 948.3, Found (M+H)⁺ m/z: 948.5

### Compound A41. (2S,4R)-1-((S)-2-((1r,4S)-4-((4-(4′-bromo-5′-oxo-5′H-spiro[cyclohexane-1,7′-indolo[1,2-a]quinazolin]-10′-yl)piperidin-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide

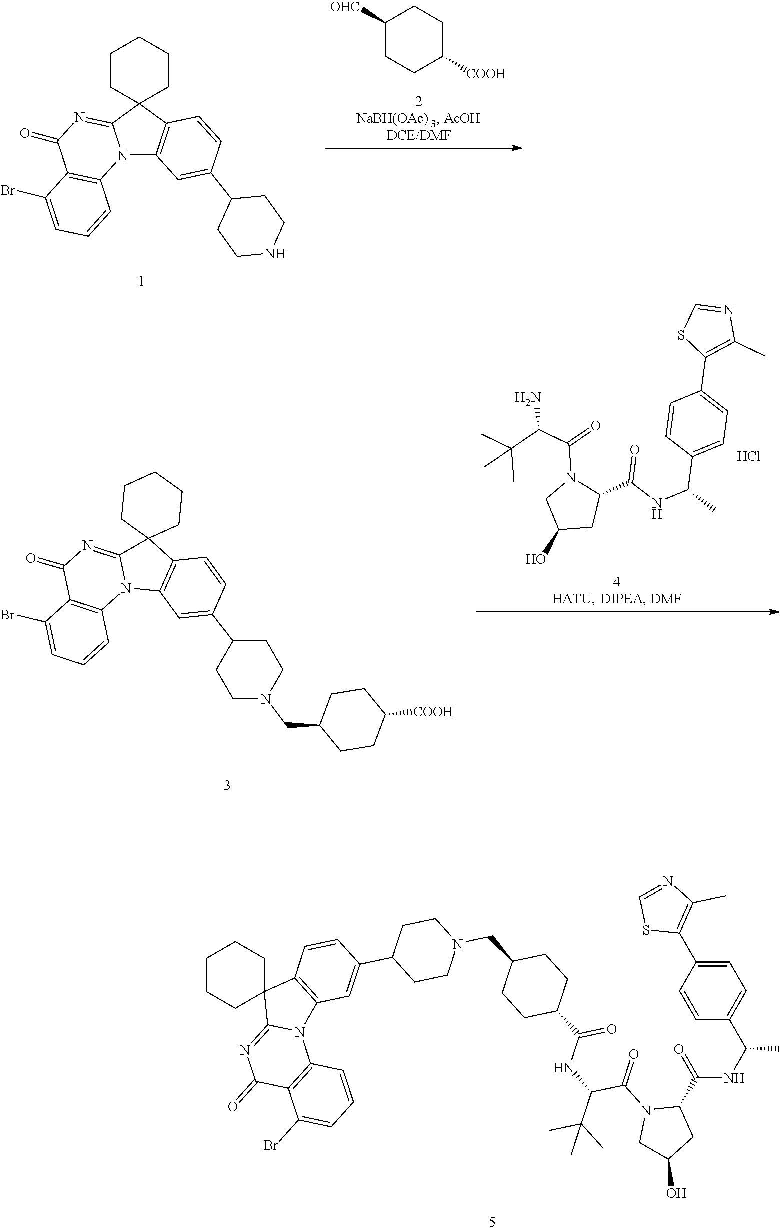

Step 1: To a stirred solution of compound 1 (13 mg 0.023 mmol, 1.0 eq) and compound 2 (7 mg, 0.045 mmol, 2.0 eq) in DMF (0.5 mL) and DCE (0.5 mL) was added acetic acid (0.011 mL, 10 eq) and the reaction mixture was stirred at room temperature for 15 minutes. Then NaBH(OAc)₃ (14.3 mg, 0.068 mmol, 3.0 eq) was added and the reaction mixture was stirred for another 4 hours. The mixture was subjected to reverse phase flash chromatography (10-100% MeCN in H₂O, 0.1% TFA) to give compound 3 (7.9 mg, 49%).

Step 2: To a stirred solution of compound 3 (7.9 mg, 0.011 mmol, 1.0 eq), compound 4 (5.3 mg, 0.011 mg, 1.0 eq) and HATU (4.6 mg, 0.012 mmol, 1.1 eq) was added DIPEA (0.017 mL, 0.11 mmol, 10 eq) and the reaction mixture was stirred for 30 minutes. The mixture was subjected to Prep-HPLC (40-100% MeCN in H₂O, 0.1% TFA) to give compound 5 (4.0 mg, 35%).

Calcd. (M+H)⁺ m/z: 1030.4, Found (M+H)⁺ m/z: 1030.5

### Compound A42. (2S,4R)-1-((S)-2-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide

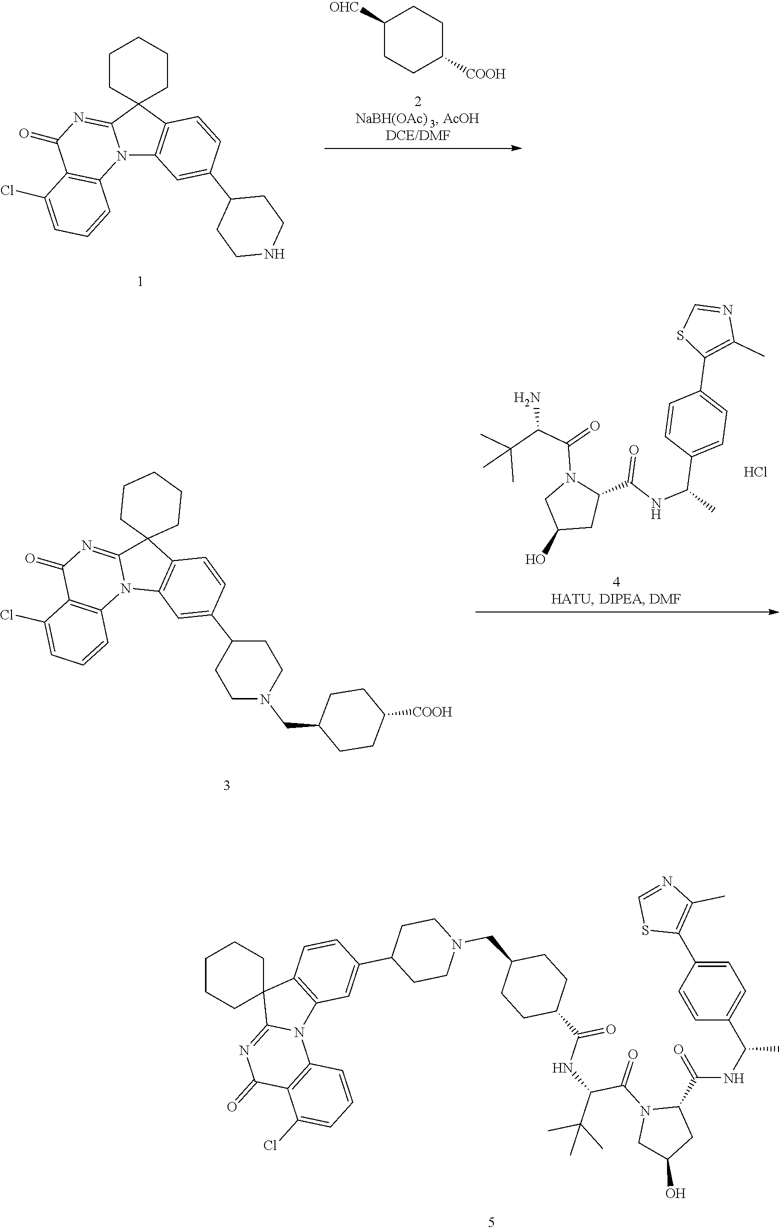

Compound A42 was synthesized using the similar procedures as used in the synthesis of Compound A41.

Calcd. (M+H)⁺ m/z: 986.5, Found (M+H)⁺ m/z: 986.5

### Compound A43. (2S,4R)-1-((S)-2-(4-((4-(4′-bromo-5′-oxo-5′H-spiro[cyclohexane-1,7′-indolo[1,2-a]quinazolin]-10′-yl)piperidin-1-yl)methyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

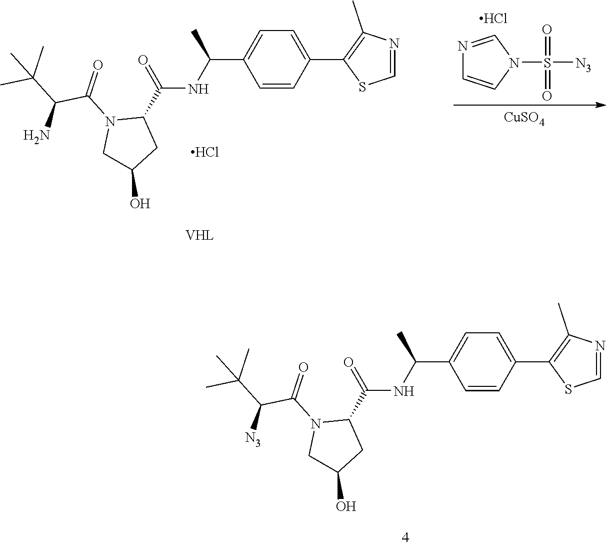

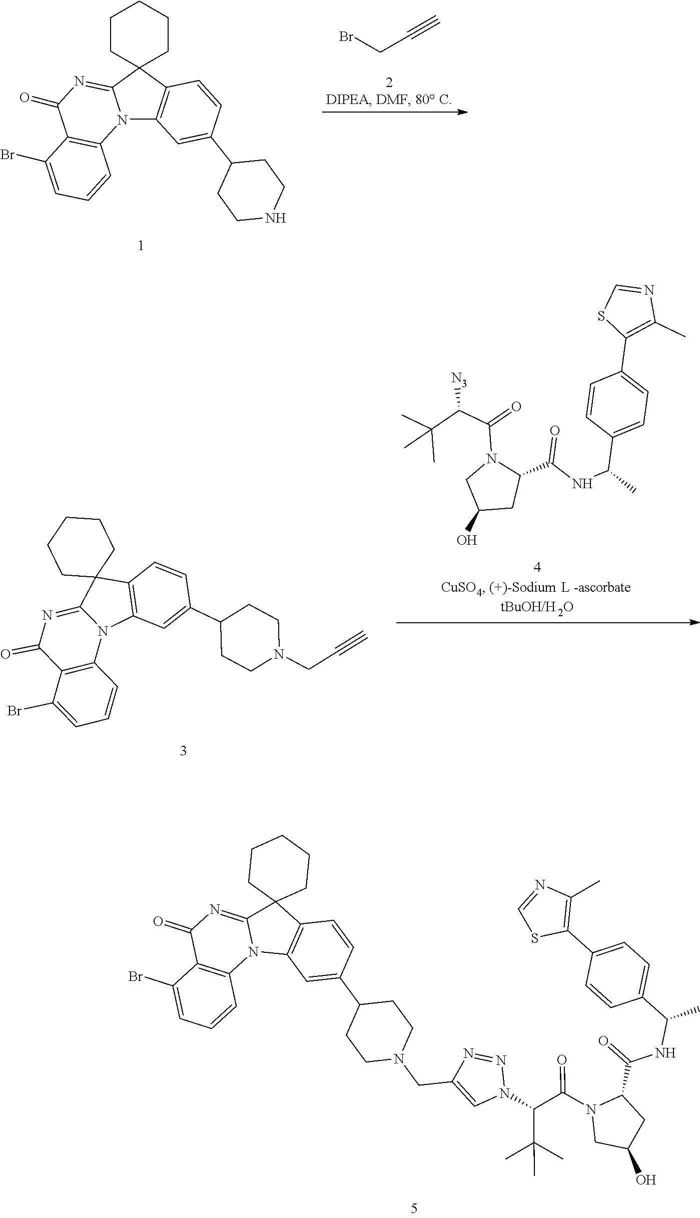

Step 1: To a flask was added VHL (818 mg, 1.7 mmol, 1.0 eq), 1H-imidazole-1-sulfonyl azide hydrochloride (391 mg, 1.87 mmol, 1.1 eq), K₂CO₃ (586 mg, 4.25 mmol, 2.5 eq), CuSO₄ (27 mg, 0.17 mmol, 0.1 eq). Then the solvent of MeOH (9 mL) was added to the flask. The suspension was stirred at rt for 8 h. After removing solvent, the residue was purified directly by C-18 reversal column chromatography (elution solvents: CH₃CN/H₂O from 10% to 100%, 0.1% TFA) to afford compound 4 (875 mg, 88%) as a withe solid.

Step 2: To a stirred solution of compound 1 (20 mg 0.035 mmol, 1.0 eq) and compound 2 (4.6 mg, 0.038 mmol, 1.1 eq) in DMF (1 mL) was added DIPEA (0.054 mL, 10 eq). The reaction mixture was stirred at 80° C. for 12 hours. After cooling to room temperature, the mixture was subjected to reverse phase flash chromatography (10-100% MeCN in H₂O, 0.1% TFA) to give compound 3 (3.0 mg, 14%).

Step 3: To a stirred solution of compound 3 (3.0 mg, 0.0048 mmol, 1.0 eq), compound 4 (3.4 mg, 0.0058 mg, 1.2 eq) in tBuOH (0.5 mL) and water (0.5 mL) was added CuSO₄ (1.0 mg, 0.0063 mmol, 1.3 eq) and (+)-Sodium L-ascorbate (3.9 mg, 0.019 mmol, 4.0 eq). The reaction mixture was stirred at room temperature for 12 hours. The mixture was subjected to Prep-HPLC (40-100% MeCN in H₂O, 0.1% TFA) to give compound 5 (2.1 mg, 44%).

Calcd. (M+H)⁺ m/z: 972.4, Found (M+H)⁺ m/z: 972.5

### Compound A44. (2S,4R)-1-((S)-2-(4-(3-(4-(4′-bromo-5′-oxo-5′H-spiro[cyclohexane-1,7′-indolo[1,2-a]quinazolin]-10′-yl)piperidin-1-yl) propyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

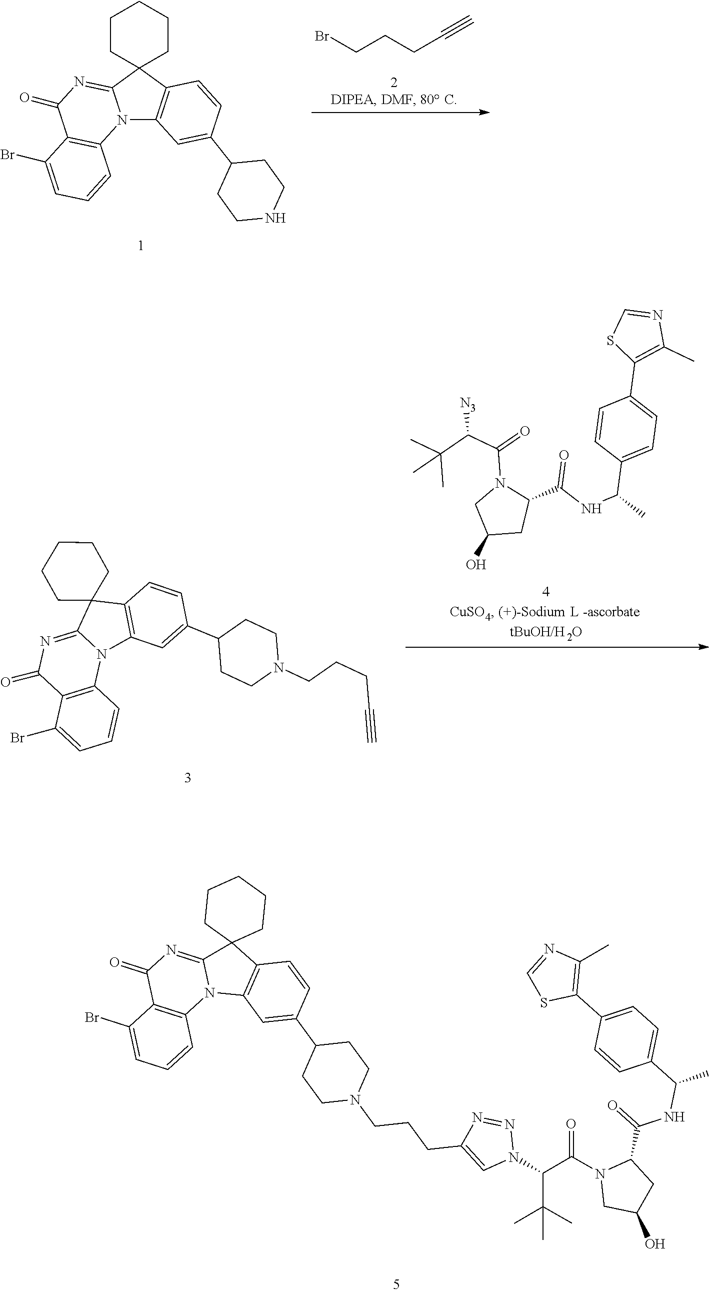

Compound A44 was synthesized using the similar procedures as used in the synthesis of Compound A43.

Calcd. (M+H)⁺ m/z: 1000.4, Found (M+H)⁺ m/z: 1000.2

### Compound A45. (2S,4R)-1-((S)-2-(4-(4-(4-(4′-bromo-5′-oxo-5′H-spiro[cyclohexane-1,7′-indolo[1,2-a]quinazolin]-10′-yl)piperidin-1-yl)butyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

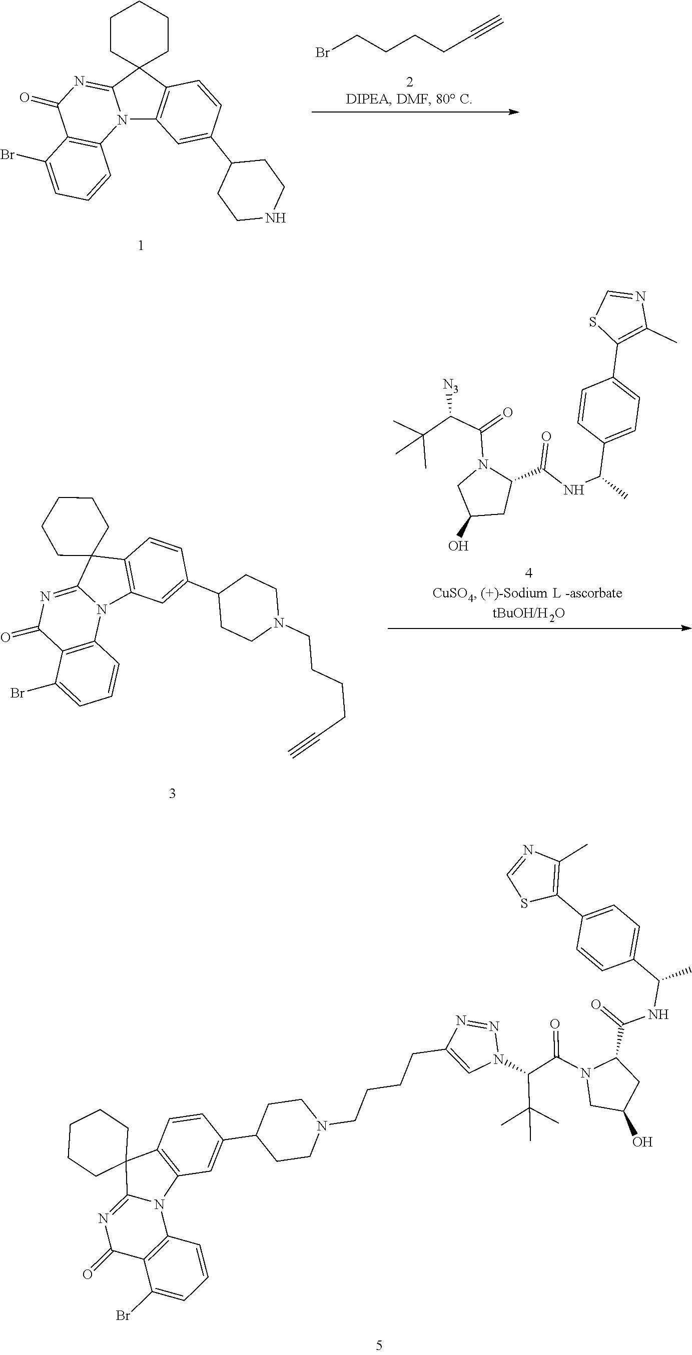

Compound A45 was synthesized using the similar procedures as used in the synthesis of Compound A43.

Calcd. (M+H)⁺ m/z: 1014.4, Found (M+H)⁺ m/z: 1014.3

### Compound A46. (2S,4R)-1-((S)-2-(4-(5-(4-(4′-bromo-5′-oxo-5′H-spiro[cyclohexane-1,7′-indolo[1,2-a]quinazolin]-10′-yl)piperidin-1-yl) pentyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

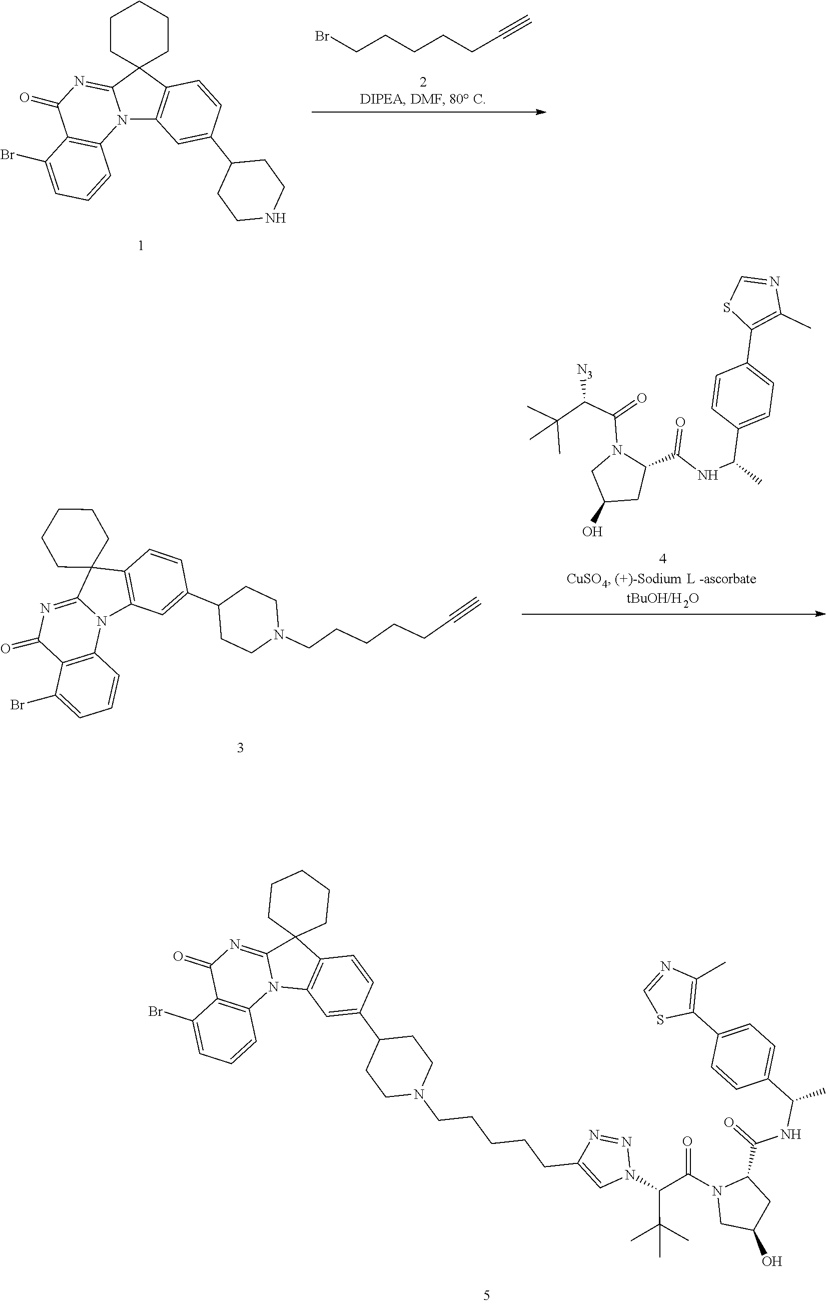

Compound A46 was synthesized using the similar procedures as used in the synthesis of Compound A43.

Calcd. (M+H)⁺ m/z: 1028.4, Found (M+H)⁺ m/z: 1028.3

### Compound A47. (2S,4R)-1-((S)-2-(4-(6-(4-(4′-bromo-5′-oxo-5′H-spiro[cyclohexane-1,7′-indolo[1,2-a]quinazolin]-10′-yl)piperidin-1-yl) hexyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

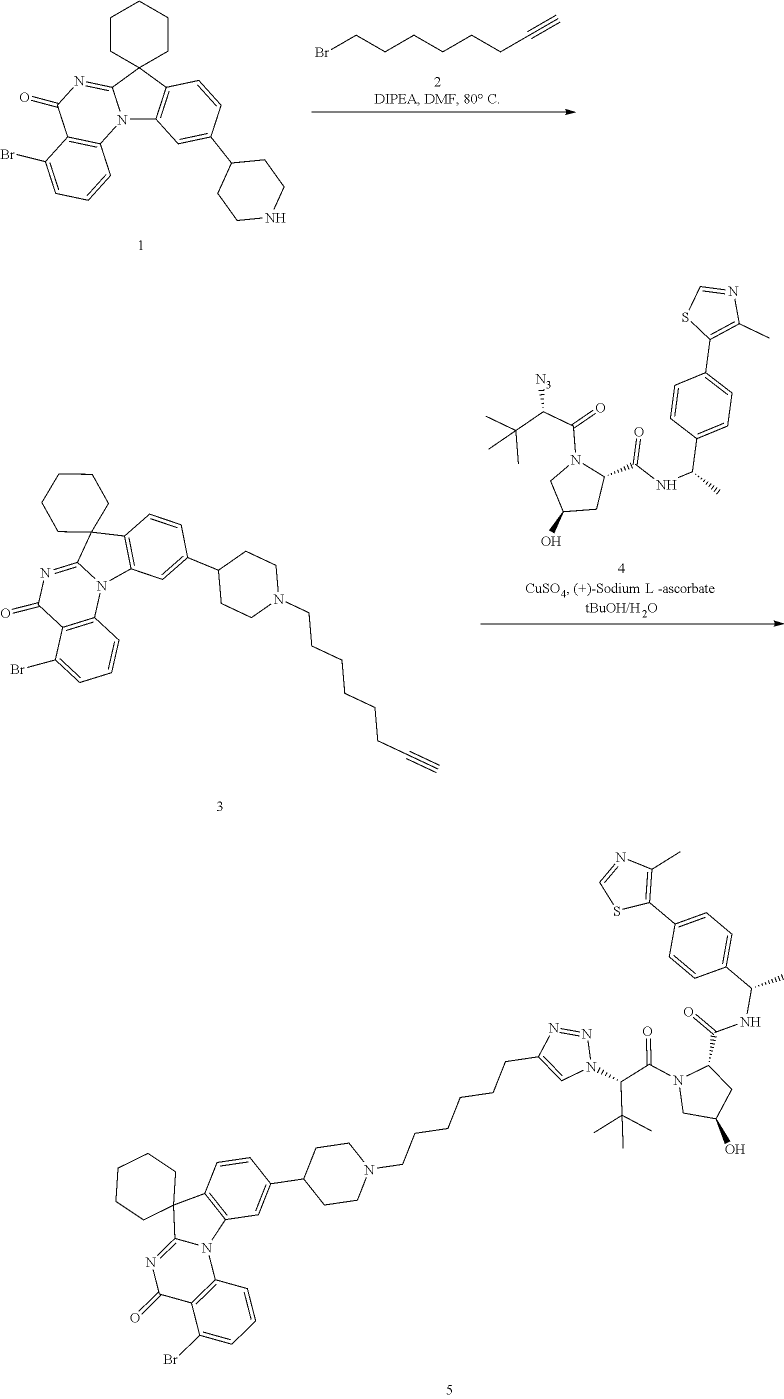

Compound A47 was synthesized using the similar procedures as used in the synthesis of Compound A43.

Calcd. (M+H)⁺ m/z: 1042.4, Found (M+H)⁺ m/z: 1042.6

### Compound A48. (2S,4R)-1-((S)-2-(4-(2-(4-(4′-bromo-5′-oxo-5′H-spiro[cyclohexane-1,7′-indolo[1,2-a]quinazolin]-10′-yl)piperidin-1-yl)ethyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

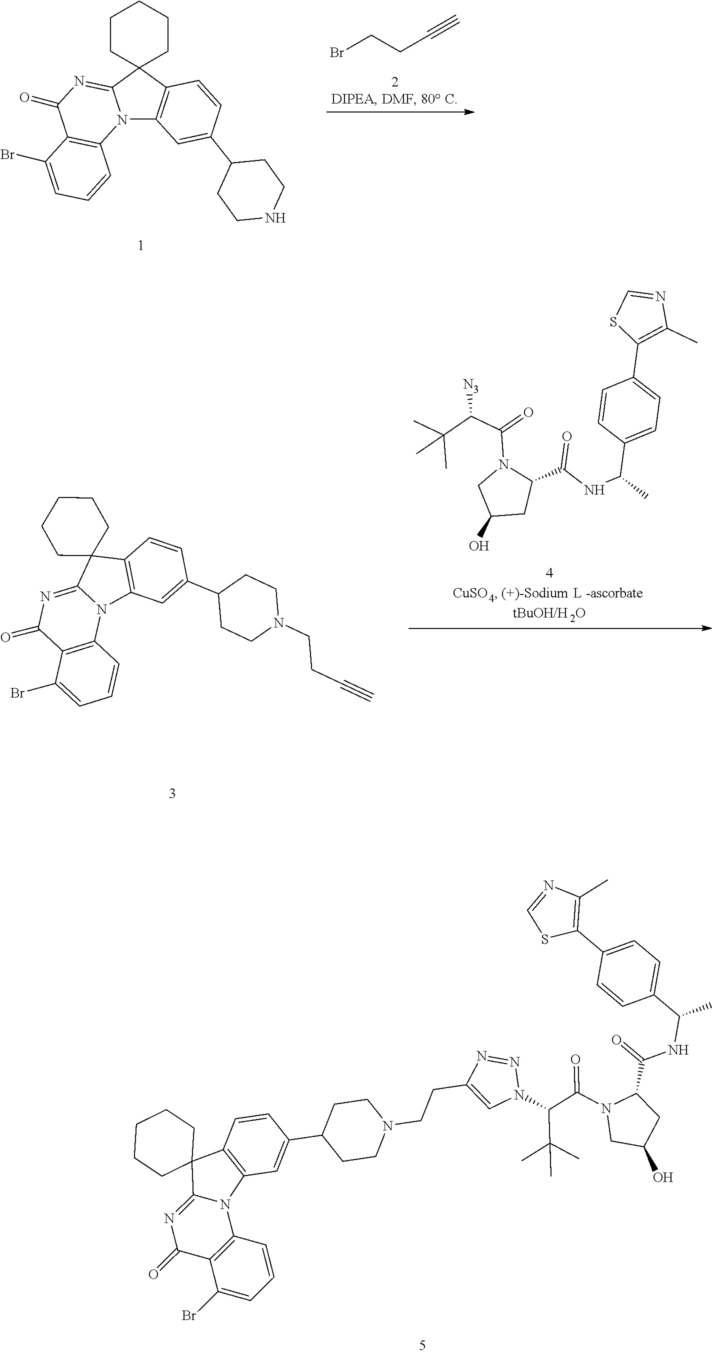

Compound A48 was synthesized using the similar procedures as used in the synthesis of Compound A43.

Calcd. (M+H)⁺ m/z: 986.4, Found (M+H)⁺ m/z: 986.5

### Compound A49. (2S,4R)-1-((S)-2-(4-((1r,4S)-4-((4-(4′-bromo-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

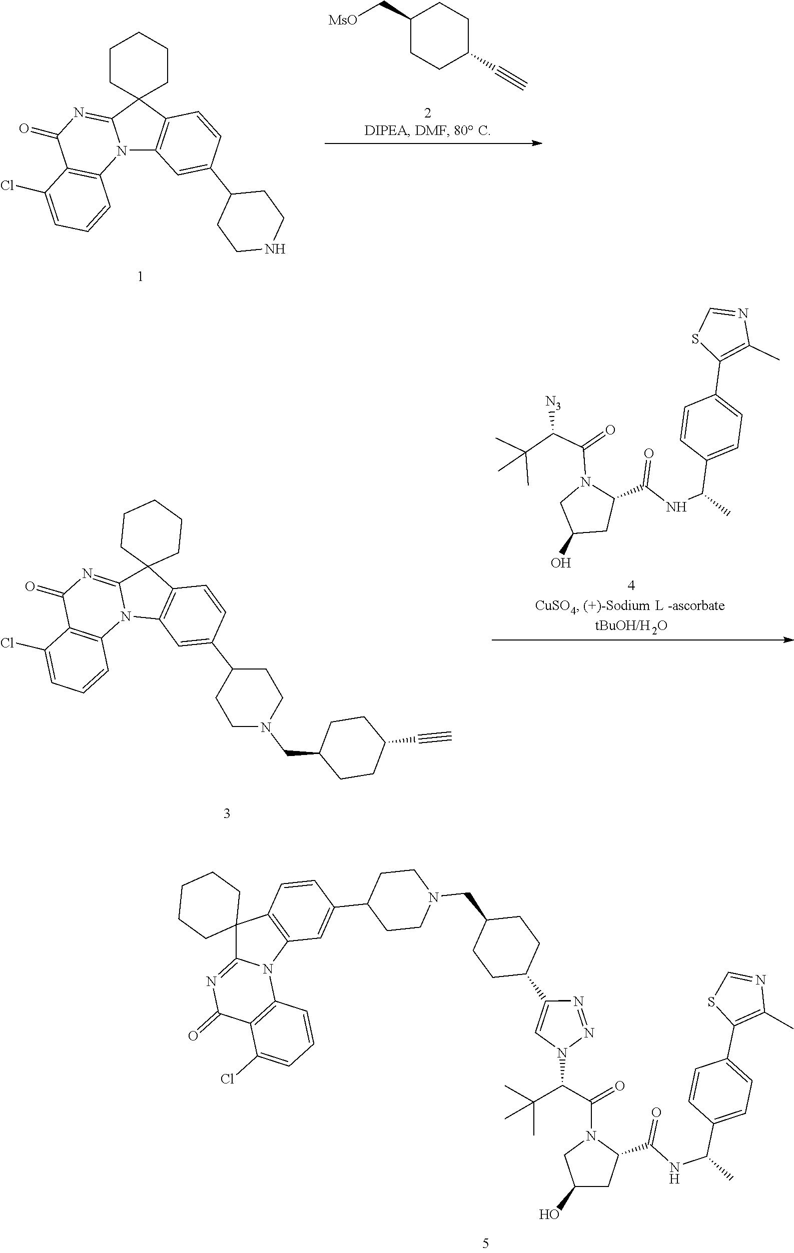

Step 1: To a stirred solution of compound 1 (50 mg 0.086 mmol, 1.0 eq) and compound 2 (37.3 mg, 0.172 mmol, 2.0 eq) in DMF (1 mL) was added DIPEA (0.133 mL, 10 eq) and the reaction mixture was stirred at 90° C. for 12 hours. After cooling to room temperature, the mixture was subjected to reverse phase flash chromatography (10-100% MeCN in H₂O, 0.1% TFA) to give compound 3 (16.6 mg, 30%).

Step 2: To a stirred solution of compound 3 (16.6 mg, 0.025 mmol, 1.0 eq), compound 4 (17.8 mg, 0.030 mg, 1.2 eq) in tBuOH (0.5 mL) and water (0.5 mL) was added CuSO₄ (5.3 mg, 0.033 mmol, 1.3 eq) and (+)-Sodium L-ascorbate (20.1 mg, 0.101 mmol, 4.0 eq). The reaction mixture was stirred at room temperature for 12 hours. The mixture was subjected to Prep-HPLC (35-100% MeCN in H₂O, 0.1% TFA) to give compound 5 (17.0 mg, 67%).

Calcd. (M+H)⁺ m/z: 1054.4, Found (M+H)⁺ m/z: 1054.2

### Compound A50. (2S,4R)-1-((S)-2-(4-((1r,4S)-4-((4-(4′-bromo-5′-oxo-5′H-spiro[cyclohexane-1,7′-pyrido[3′,2′: 4,5]pyrrolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

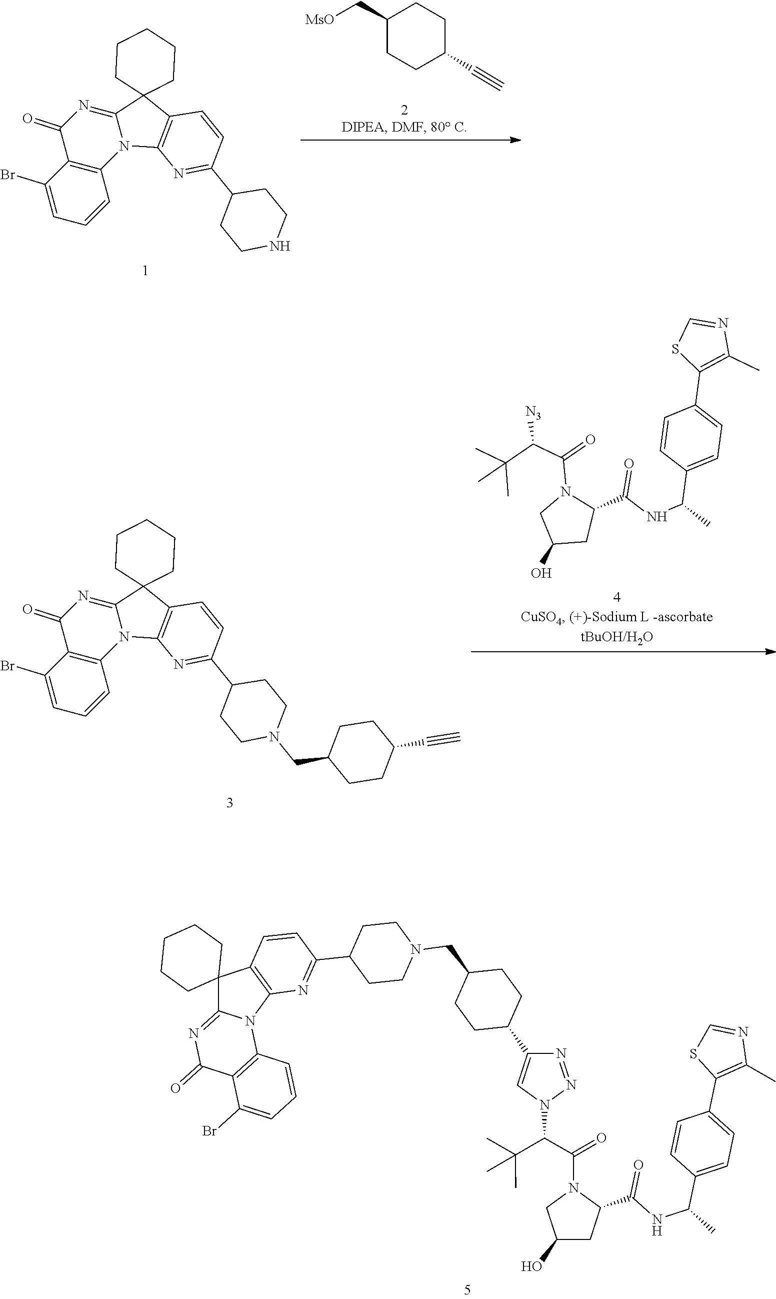 [SMILES: O=c1nc2n(c3cccc(Br)c13)-c1nc(C3CCNCC3)ccc1C21CCCCC1 ; C#C[C@H]1CC[C@H](COS(C)(=O)=O)CC1 ; C#C[C@H]1CC[C@H](CN2CCC(c3ccc4c(n3)-n3c(nc(=O)c5c(Br)cccc53)C43CCCCC3)CC2)CC1 ; Cc1ncsc1-c1ccc([C@H](C)NC(=O)[C@@H]2C[C@@H](O)CN2C(=O)[C@@H](N=[N+]=[N-])C(C)(C)C)cc1 ; Cc1ncsc1-c1ccc([C@H](C)NC(=O)[C@@H]2C[C@@H](O)CN2C(=O)[C@@H](n2cc([C@H]3CC[C@H](CN4CCC(c5ccc6c(n5)-n5c(nc(=O)c7c(Br)cccc75)C65CCCCC5)CC4)CC3)nn2)C(C)(C)C)cc1]

Compound A50 was synthesized using the similar procedures as used in the synthesis of Compound A49.

Calcd. (M+H)⁺ m/z: 1055.4, Found (M+H)⁺ m/z: 1055.3

### Compound A51. (2S,4R)-1-((S)-2-((1r,4S)-4-((4-(4′-bromo-5′-oxo-5′H-spiro[cyclohexane-1,7′-pyrido[3′,2′: 4,5]pyrrolo[1,2-a]quinazolin]-10′-yl)piperidin-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide

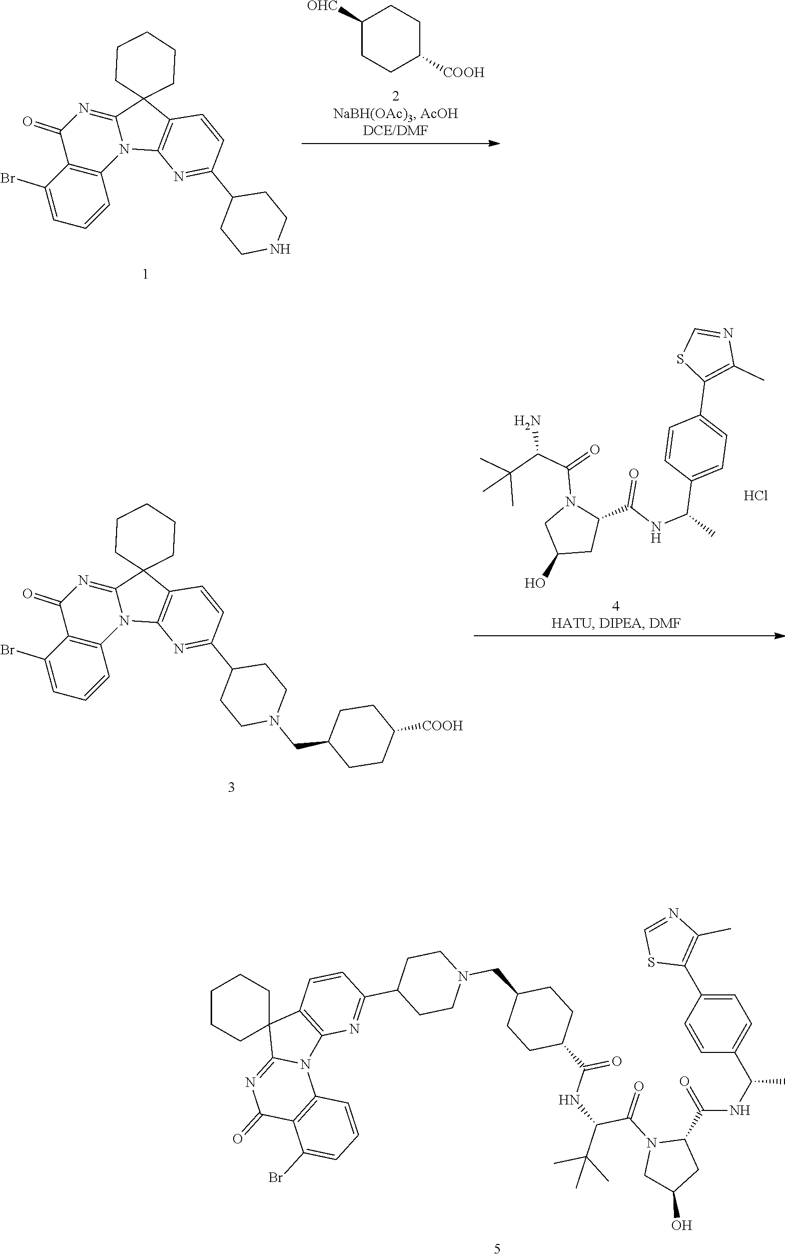 [SMILES: O=c1nc2n(c3cccc(Br)c13)-c1nc(C3CCNCC3)ccc1C21CCCCC1 ; O=C[C@H]1CC[C@H](C(=O)O)CC1 ; O=c1nc2n(c3cccc(Br)c13)-c1nc(C3CCN(C[C@H]4CC[C@H](C(=O)O)CC4)CC3)ccc1C21CCCCC1 ; Cc1ncsc1-c1ccc([C@H](C)NC(=O)[C@@H]2C[C@@H](O)CN2C(=O)[C@@H](N)C(C)(C)C)cc1 ; Cl ; Cc1ncsc1-c1ccc([C@H](C)NC(=O)[C@@H]2C[C@@H](O)CN2C(=O)[C@@H](NC(=O)[C@H]2CC[C@H](CN3CCC(c4ccc5c(n4)-n4c(nc(=O)c6c(Br)cccc64)C54CCCCC4)CC3)CC2)C(C)(C)C)cc1]

Compound A51 was synthesized using the similar procedures as used in the synthesis of Compound A41.

Calcd. (M+H)⁺ m/z: 1031.4, Found (M+H)⁺ m/z: 1031.4

### Compound A52. (2S,4R)-1-((S)-2-(4-((1r,4S)-4-((4-(4′-bromo-5′-oxo-5′H-spiro[cyclohexane-1,7′-pyrido[3′,2′: 4,5]pyrrolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-1-(4-(4-methylthiazol-5-yl)phenyl)-2-morpholinoethyl)pyrrolidine-2-carboxamide

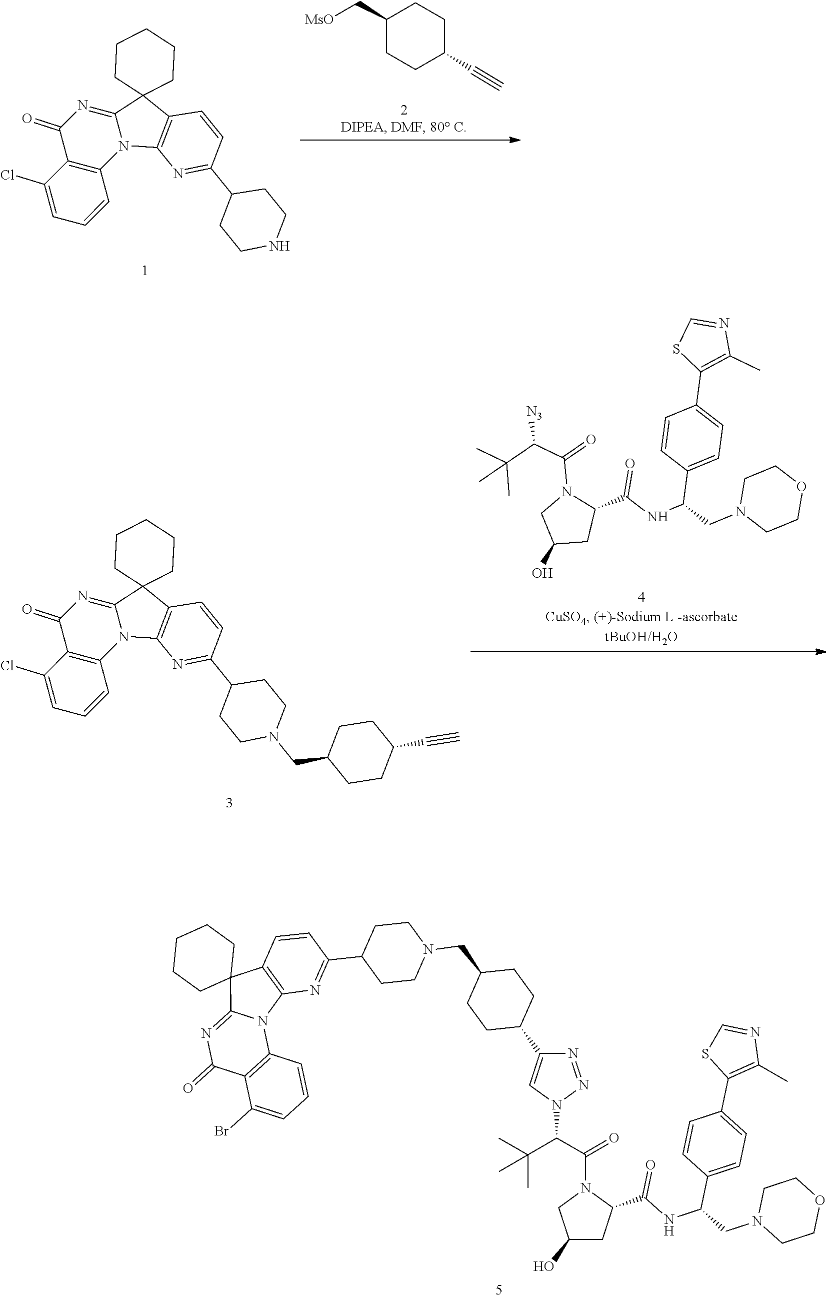 [SMILES: O=c1nc2n(c3cccc(Cl)c13)-c1nc(C3CCNCC3)ccc1C21CCCCC1 ; C#C[C@H]1CC[C@H](COS(C)(=O)=O)CC1 ; C#C[C@H]1CC[C@H](CN2CCC(c3ccc4c(n3)-n3c(nc(=O)c5c(Cl)cccc53)C43CCCCC3)CC2)CC1 ; Cc1ncsc1-c1ccc([C@H](CN2CCOCC2)NC(=O)[C@@H]2C[C@@H](O)CN2C(=O)[C@@H](N=[N+]=[N-])C(C)(C)C)cc1 ; Cc1ncsc1-c1ccc([C@H](CN2CCOCC2)NC(=O)[C@@H]2C[C@@H](O)CN2C(=O)[C@@H](n2cc([C@H]3CC[C@H](CN4CCC(c5ccc6c(n5)-n5c(nc(=O)c7c(Br)cccc75)C65CCCCC5)CC4)CC3)nn2)C(C)(C)C)cc1]

Compound A52 was synthesized using the similar procedures as used in the synthesis of Compound A49.

Calcd. (M+H)⁺ m/z: 1140.5, Found (M+H)⁺ m/z: 1140.6

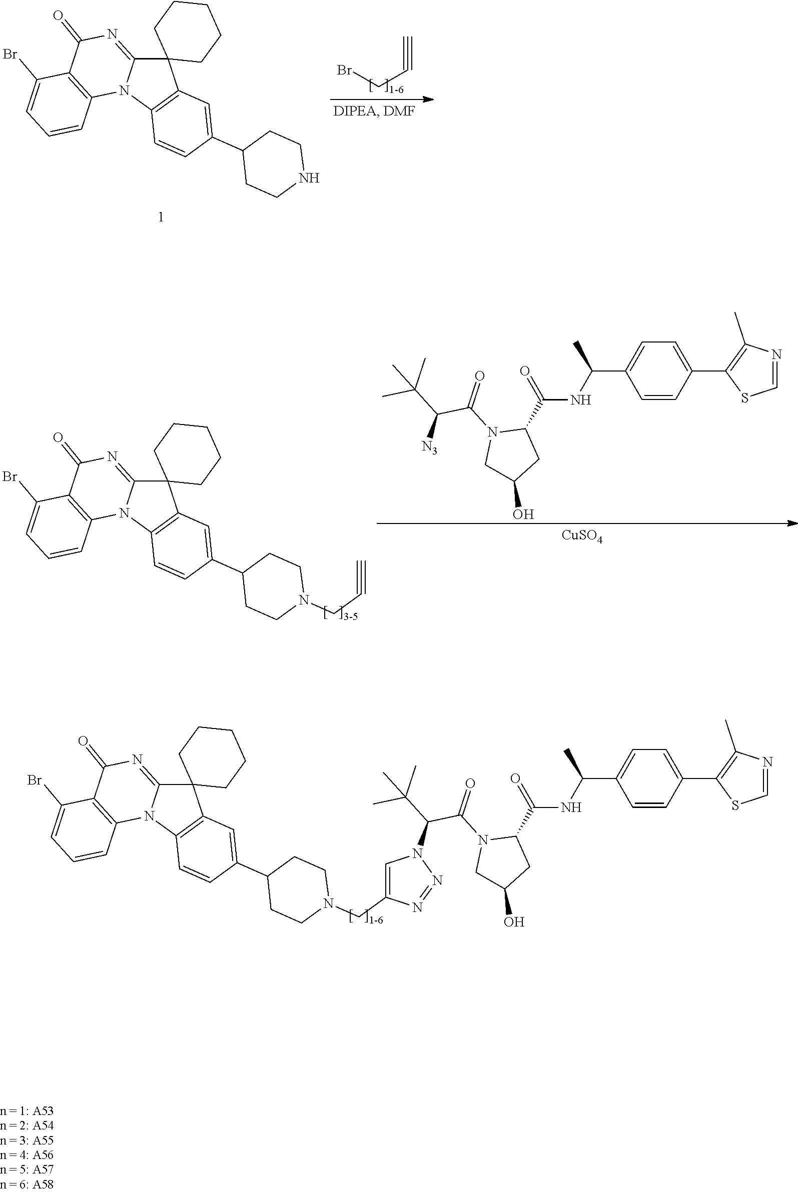

### Compound A53. (2S,4R)-1-((S)-2-(4-((4-(4′-bromo-5′-oxo-5′H-spiro[cyclohexane-1,7′-indolo[1,2-a]quinazolin]-9′-yl)piperidin-1-yl)methyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

Compound A53 was synthesized using the similar procedures as used in the synthesis of Compound A43. ESI MS m/z: 972.15 [M+H]⁺.

### Compound A54. (2S,4R)-1-((S)-2-(4-(2-(4-(4′-bromo-5′-oxo-5′H-spiro[cyclohexane-1,7′-indolo[1,2-a]quinazolin]-9′-yl)piperidin-1-yl)ethyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

Compound A54 was synthesized using the similar procedures as used in the synthesis of Compound A43. ESI MS m/z: 986.41 [M+H]⁺.

### Compound A55. (2S,4R)-1-((S)-2-(4-(3-(4-(4′-bromo-5′-oxo-5′H-spiro[cyclohexane-1,7′-indolo[1,2-a]quinazolin]-9′-yl)piperidin-1-yl) propyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

Compound A55 was synthesized using the similar procedures as used in the synthesis of Compound A43. ESI MS m/z: 1000.39 [M+H]⁺.

### Compound A56. (2S,4R)-1-((S)-2-(4-(4-(4-(4′-bromo-5′-oxo-5′H-spiro[cyclohexane-1,7′-indolo[1,2-a]quinazolin]-9′-yl)piperidin-1-yl)butyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

Compound A56 was synthesized using the similar procedures as used in the synthesis of Compound A43. ESI MS m/z: 1014.41 [M+H]⁺.

### Compound A57. (2S,4R)-1-((S)-2-(4-(5-(4-(4′-bromo-5′-oxo-5′H-spiro[cyclohexane-1,7′-indolo[1,2-a]quinazolin]-9′-yl)piperidin-1-yl) pentyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

Compound A57 was synthesized using the similar procedures as used in the synthesis of Compound A43. ESI MS m/z: 1028.40 [M+H]⁺.

### Compound A58. (2S,4R)-1-((S)-2-(4-(6-(4-(4′-bromo-5′-oxo-5′H-spiro[cyclohexane-1,7′-indolo[1,2-a]quinazolin]-9′-yl)piperidin-1-yl) hexyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

Compound A58 was synthesized using the similar procedures as used in the synthesis of Compound A43. ESI MS m/z: 1042.45 [M+H]⁺.

### Compound A59. (2S,4R)-1-(2-(3-(4-((4-(4′-bromo-5′-oxo-5′H-spiro[cyclohexane-1,7′-indolo[1,2-a]quinazolin]-9′-yl)piperidin-1-yl)methyl)piperidin-1-yl)isoxazol-5-yl)-3-methylbutanoyl)-4-hydroxy-N—((R)-1-(4-(4-methylthiazol-5-yl)phenyl)ethyl)pyrrolidine-2-carboxamide

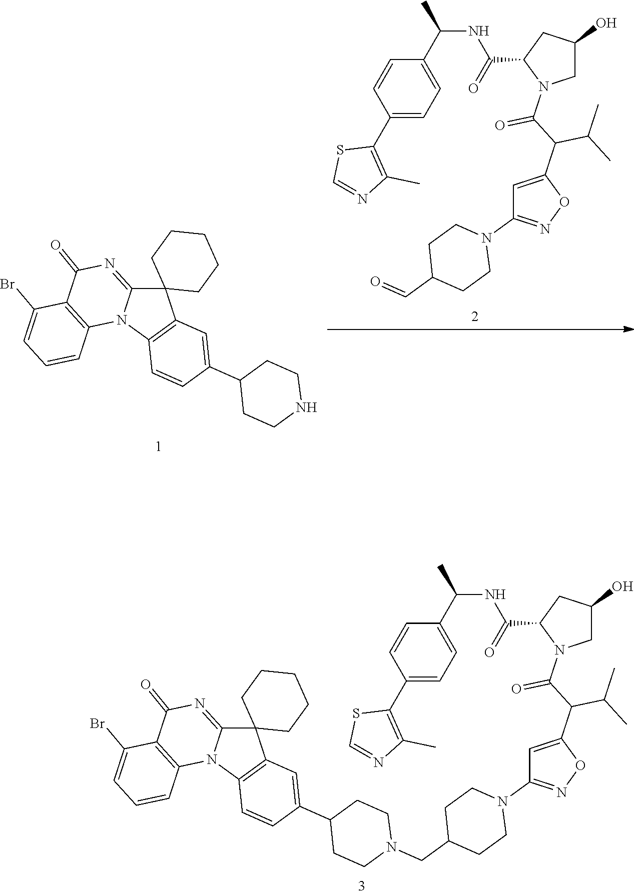 [SMILES: O=c1nc2n(c3cccc(Br)c13)-c1ccc(C3CCNCC3)cc1C21CCCCC1 ; Cc1ncsc1-c1ccc([C@@H](C)NC(=O)[C@@H]2C[C@@H](O)CN2C(=O)C(c2cc(N3CCC(C=O)CC3)no2)C(C)C)cc1 ; Cc1ncsc1-c1ccc([C@@H](C)NC(=O)[C@@H]2C[C@@H](O)CN2C(=O)C(c2cc(N3CCC(CN4CCC(c5ccc6c(c5)C5(CCCCC5)c5nc(=O)c7c(Br)cccc7n5-6)CC4)CC3)no2)C(C)C)cc1]

To a solution of 1 (9.8 mg, 0.017 mmol, 1.2 equiv.) and 2 (10 mg, 0.014 mmol, 1 equiv.) in MeOH (0.5 mL) was added NaOAc (3.4 mg, 0.042 mmol, 3.0 equiv). The mixture was stirred for 0.5 h and followed by addition of NaBH₃CN (4.4 mg, 0.07 mmol, 5.0 equiv). The mixture was stirred for 5 h. After removing solvent, the received crude product was purified by pre-HPLC to get compound 3 (8.0 mg, 45%). ESI MS m/z: 1041.40 [M+H]⁺.

### Compound A60. (2S,4R)-1-((S)-2-(4-((1r,4S)-4-((4-(4′-bromo-5′-oxo-5′H-spiro[cyclohexane-1,7′-indolo[1,2-a]quinazolin]-9′-yl)piperidin-1-yl)methyl)cyclohexyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

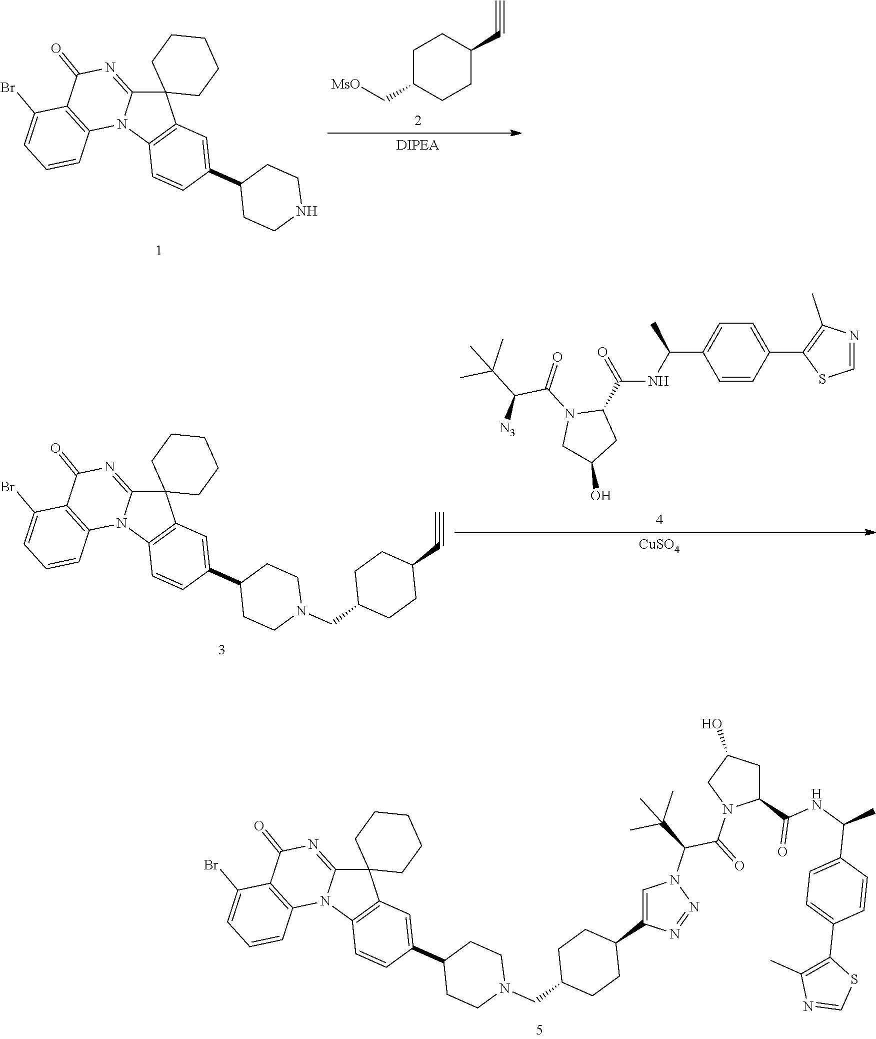

Compound A60 was synthesized using the similar procedures as used in the synthesis of Compound A49. ESI MS m/z: 1054.45 [M+H]⁺.

### Compound A61. (2S,4R)-1-((S)-2-(4-((1r,3S)-3-((4-(4′-bromo-5′-oxo-5′H-spiro[cyclohexane-1,7′-indolo[1,2-a]quinazolin]-9′-yl)piperidin-1-yl)methyl)cyclobutyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

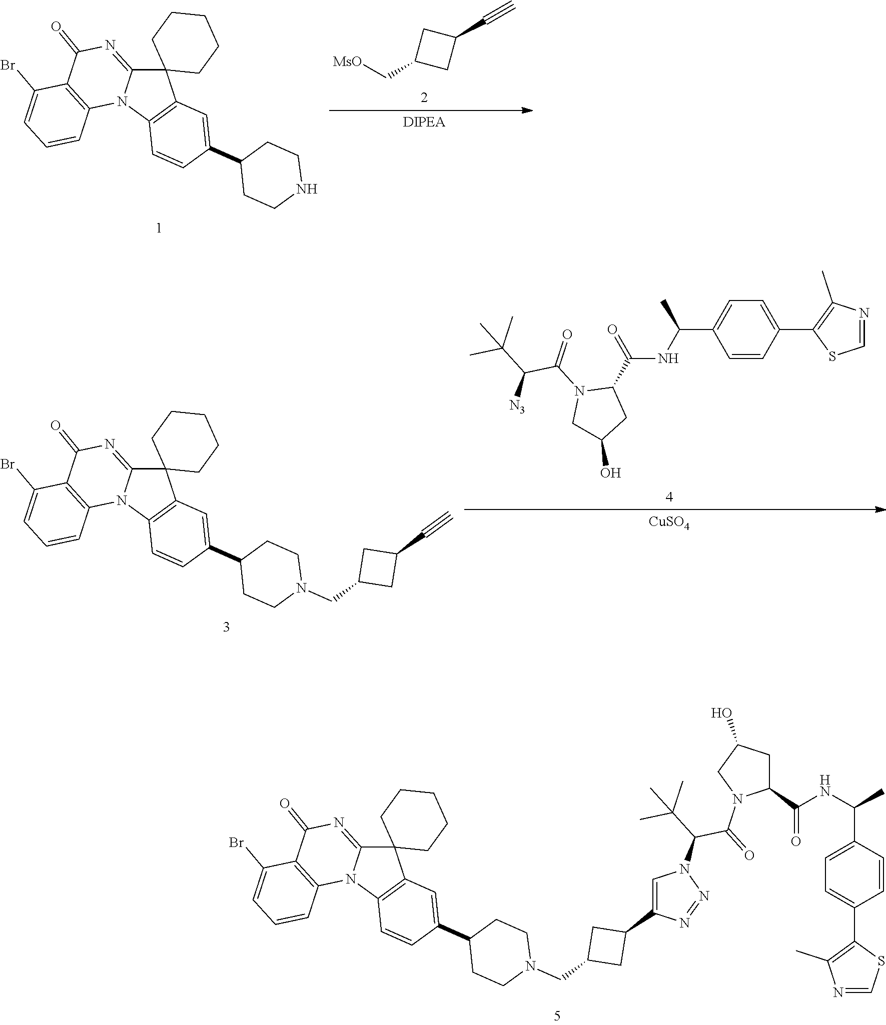

Compound A61 was synthesized using the similar procedures as used in the synthesis of Compound A49. ESI MS m/z: 1026.41 [M+H]⁺.

### Compound A62. (2S,4R)-1-((S)-2-(4-((1r,4S)-4-((4-(4′-bromo-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

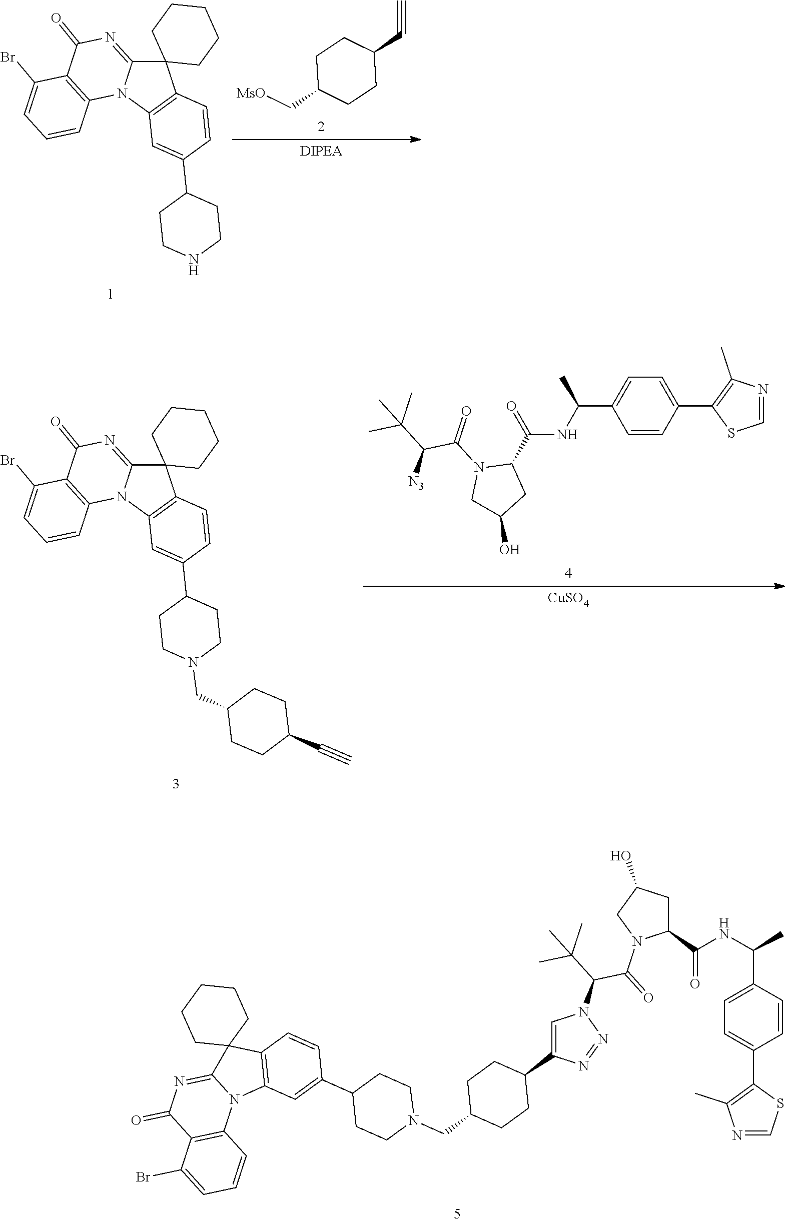

Compound A62 was synthesized using the similar procedures as used in the synthesis of Compound A49. ESI MS m/z: 1054.37 [M+H]⁺.

### Compound A63. (2S,4R)-1-((S)-2-(4-((1r,3S)-3-((4-(4′-bromo-5′-oxo-5′H-spiro[cyclohexane-1,7′-indolo[1,2-a]quinazolin]-10′-yl)piperidin-1-yl)methyl)cyclobutyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

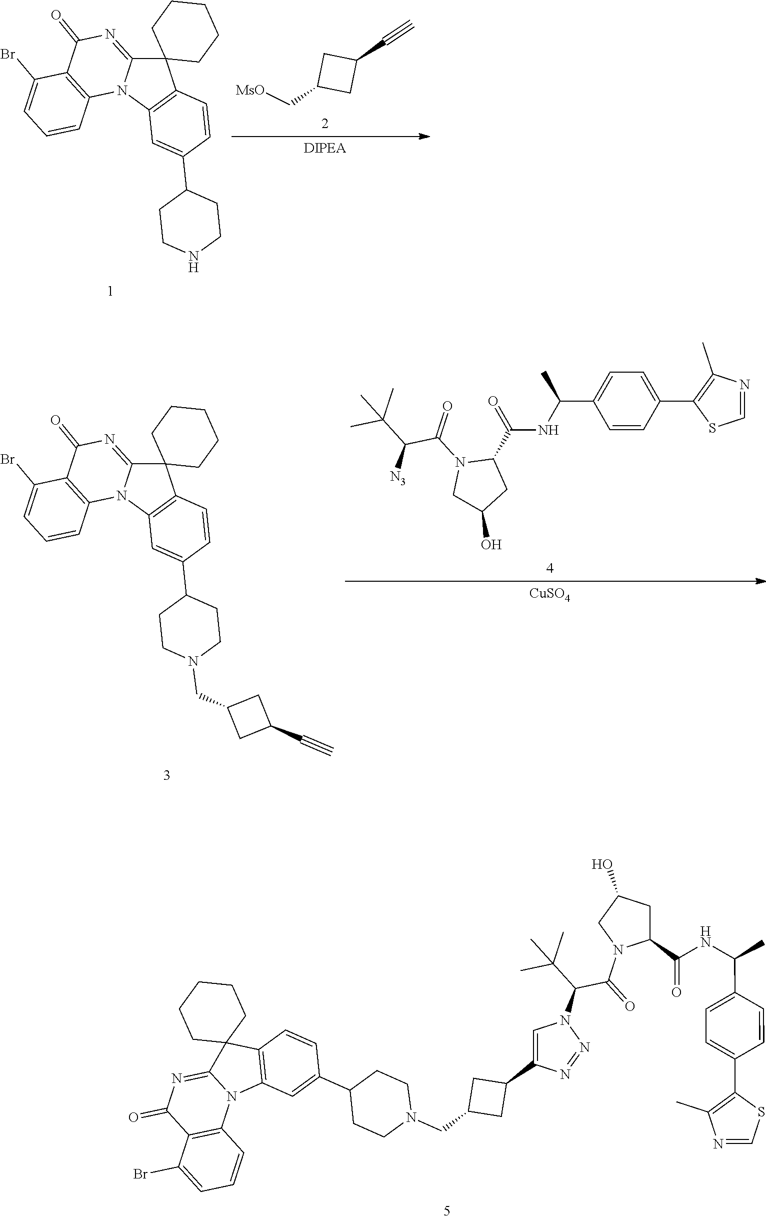

Compound A63 was synthesized using the similar procedures as used in the synthesis of Compound A49. ESI MS m/z: 1026.46 [M+H]⁺.

### Compound A64. (2S,4R)-1-((S)-2-(4-(1-((4-(4′-bromo-5′-oxo-5′H-spiro[cyclohexane-1,7′-indolo[1,2-a]quinazolin]-10′-yl)piperidin-1-yl)methyl)cyclopropyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

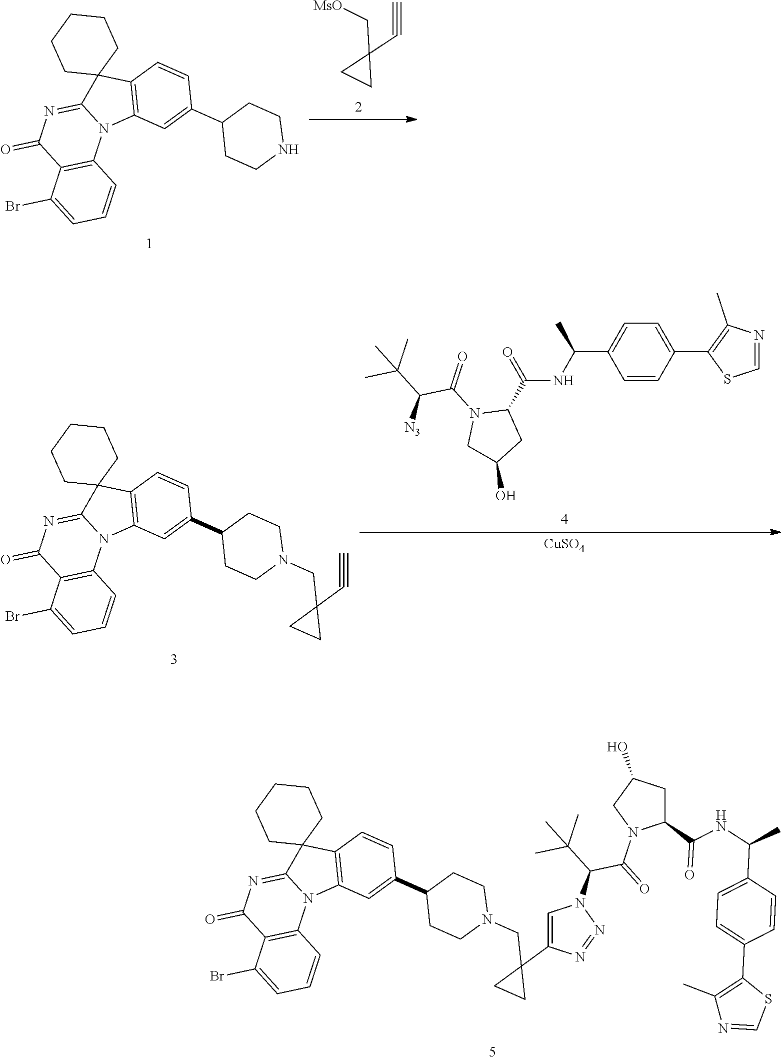

Compound A64 was synthesized using the similar procedures as used in the synthesis of Compound A49. ESI MS m/z: 1012.39 [M+H]⁺.

### Compound A65. (2S,4R)-1-((S)-2-(4-(1-(2-(4-(4′-bromo-5′-oxo-5′H-spiro[cyclohexane-1,7′-indolo[1,2-a]quinazolin]-10′-yl)piperidin-1-yl)ethyl)cyclopropyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

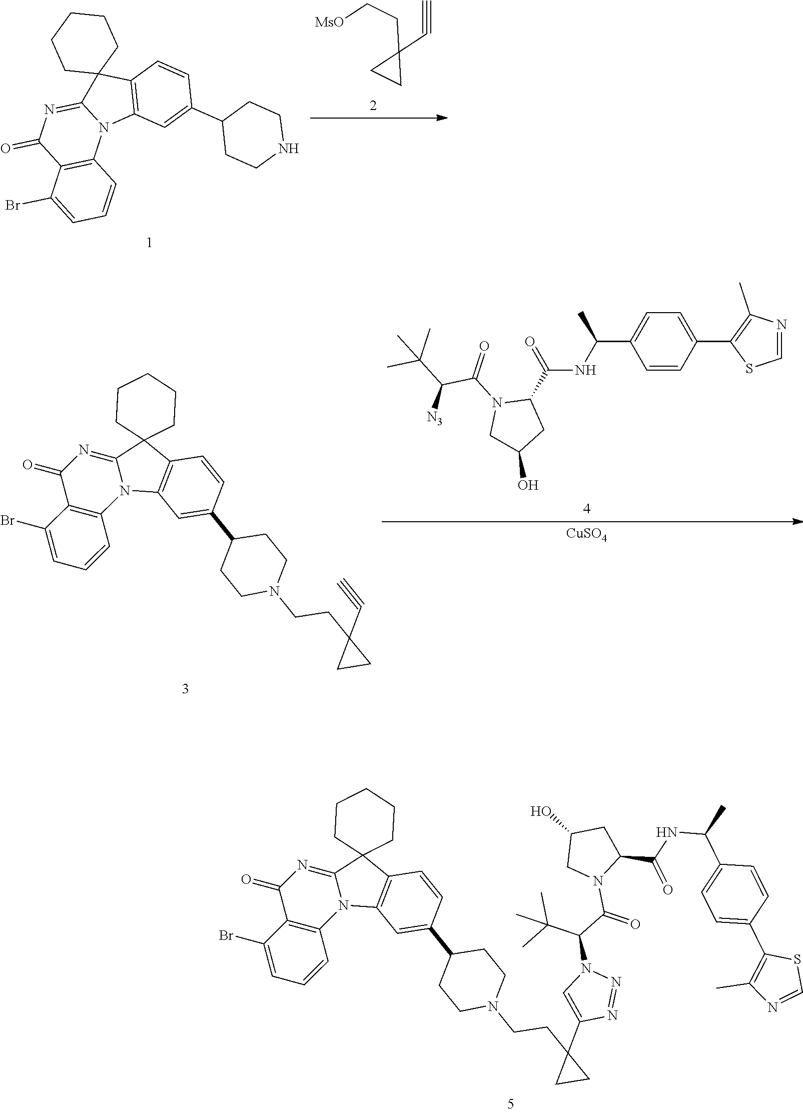

Compound A65 was synthesized using the similar procedures as used in the synthesis of Compound A49. ESI MS m/z: 1026.27 [M+H]⁺.

### Compound A66. (2S,4R)-1-((S)-2-(4-(1-((4-(4′-bromo-5′-oxo-5′H-spiro[cyclohexane-1,7′-indolo[1,2-a]quinazolin]-10′-yl)piperidin-1-yl)methyl)cyclopropyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

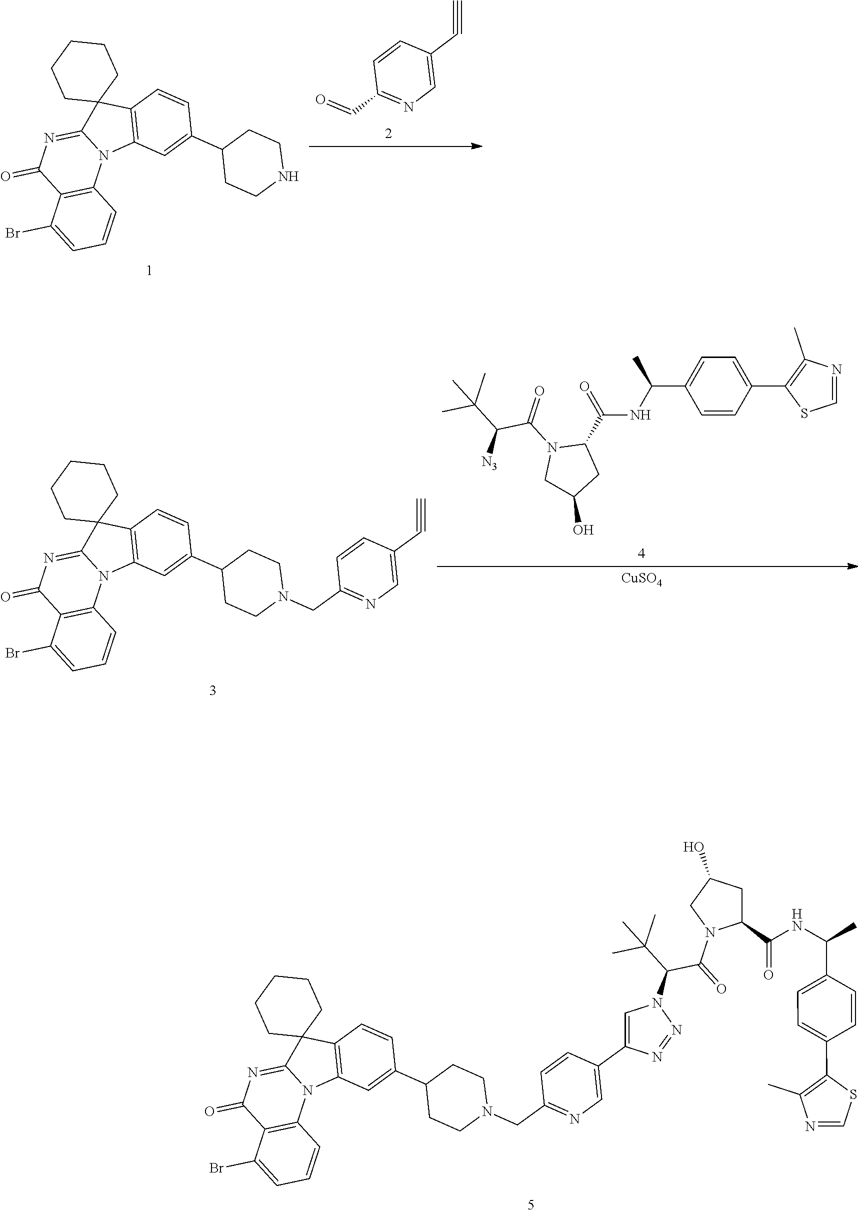

Compound A66 was synthesized using the similar procedures as used in the synthesis of Compound A49. ESI MS m/z: 1049.46 [M+H]⁺.

### Compound A67. (2S,4R)-1-((S)-2-(4-((1r,4S)-4-((4-(4′-bromo-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N-(4-(4-methylthiazol-5-yl)benzyl)pyrrolidine-2-carboxamide

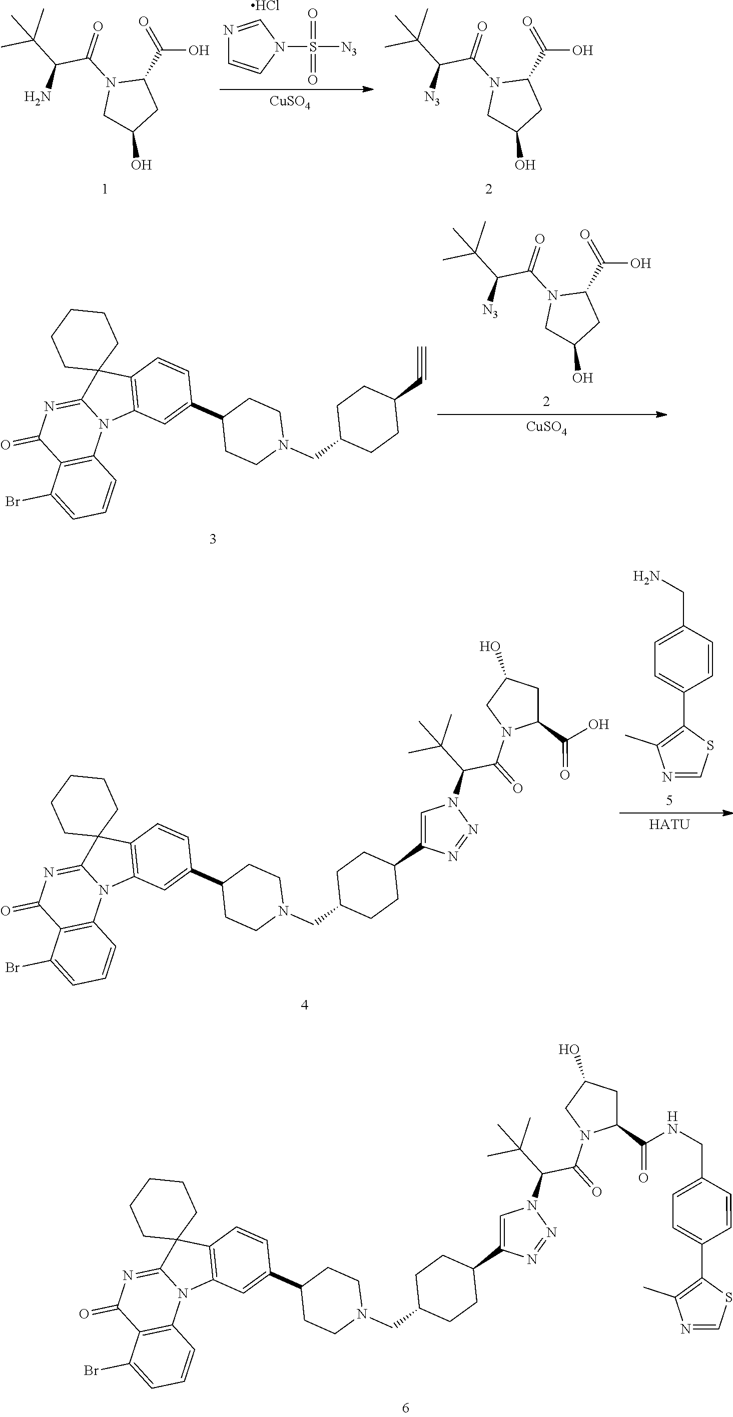

### Step 1:

To a flask was added int. 1 (416 mg, 1.7 mmol, 1.0 eq), 1H-imidazole-1-sulfonyl azide hydrochloride (391 mg, 1.87 mmol, 1.1 eq), K₂CO₃ (586 mg, 4.25 mmol, 2.5 eq), CuSO₄ (27 mg, 0.17 mmol, 0.1 eq). Then the solvent of MeOH (9 mL) was added to the flask. The suspension was stirred at rt for 8 h. After removing solvent, the residue was purified directly by C-18 reversal column chromatography (elution solvents: CH₃CN/H₂O from 10% to 100%, 0.1% TFA) to afford target int. 2 (201 mg, 44%) as a withe solid.

### Step 2:

To a solution of 3 (115 mg, 0.16 mmol) in water (1 mL) and tert-butyl alcohol (1 mL) was added 2 (67 mg, 0.25 mmol), copper sulfate (39 mg, 0.25 mmol) and sodium Lascorbate (130 mg, 0.66 mmol). The reaction was stirred at 25° C. for 12 h and filtered. After removing solvent, the received crude product was purified by pre-HPLC to get pure product 4 (25 mg, 18%).

### Step 3:

To a solution of 4 (5 mg, 0.0052 mmol, 1.0 equiv.) and 5 (1.3 mg, 0.0062 mmol, 1.2 equiv.) in DMF (1 mL) was added HATU (2.1 mg, 0.0052 mmol, 1.0 equiv.) and DIPEA (7.1 μL, 0.045 mmol, 8 equiv.). The mixture was stirred for 10 min. The reaction is quenched by TFA, the residue was purified by pre-HPLC to get pure compound 6 (4.1 mg, 76%). ESI MS m/z: 1040.41 [M+H]⁺.

### Compound A68. (2S,4R)-1-((S)-2-(4-((1r,4S)-4-((4-(4′-bromo-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-N—((S)-1-(4-chlorophenyl)ethyl)-4-hydroxypyrrolidine-2-carboxamide

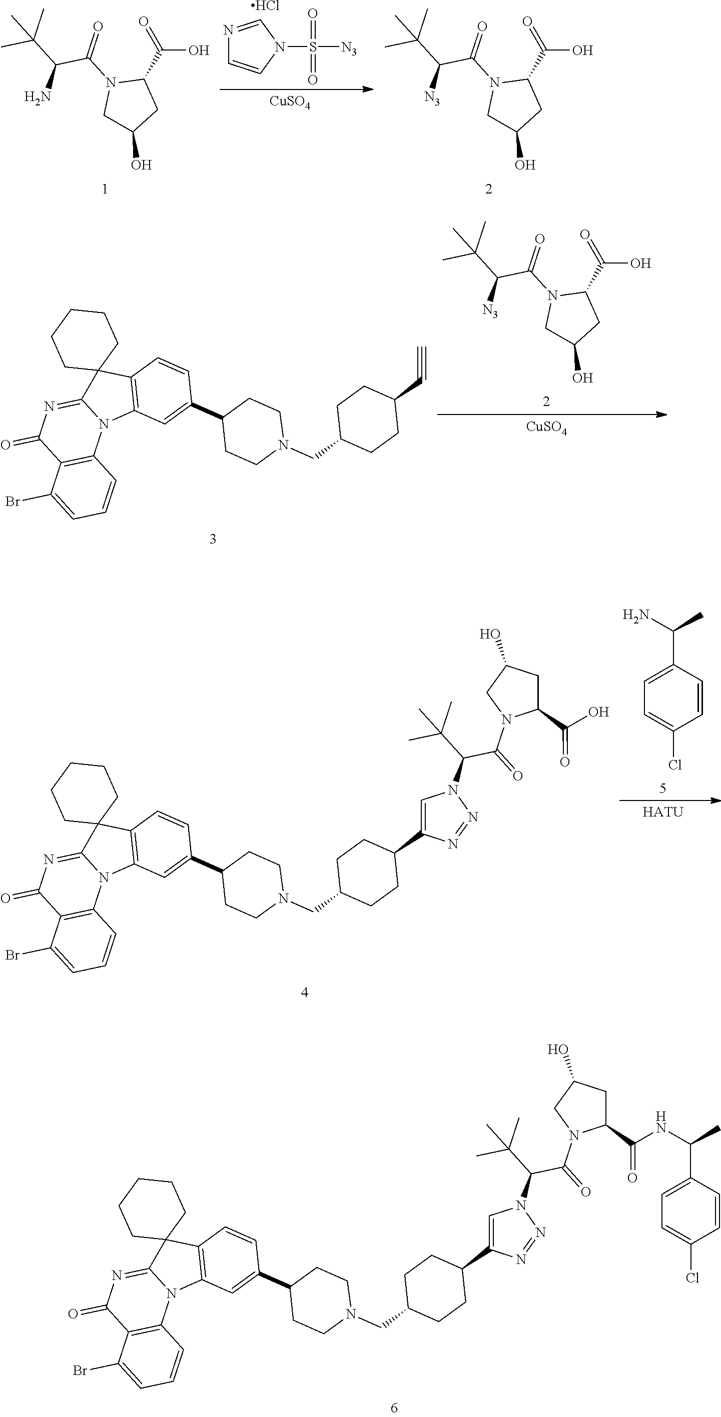

Compound A68 was synthesized using the similar procedures as used in the synthesis of Compound A67. ESI MS m/z: 991.39 [M+H]⁺.

### Compound A69. (2S,4R)-1-((S)-2-(4-((1r,4S)-4-((4-(4′-bromo-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-1-(4-(4-methylthiazol-5-yl)phenyl)-2-morpholinoethyl)pyrrolidine-2-carboxamide

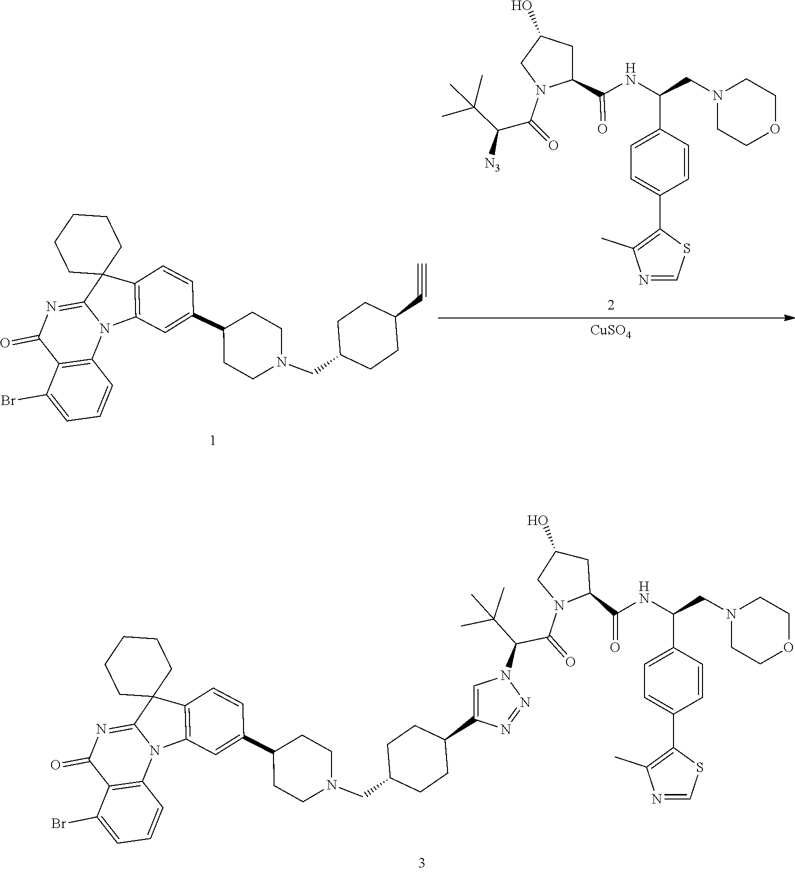

Compound A69 was synthesized using the similar procedures as used in the synthesis of Compound A49. ESI MS m/z: 1139.50 [M+H]⁺.

### Compound A70. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-1-(4-(4-methylthiazol-5-yl)phenyl)-2-morpholinoethyl)pyrrolidine-2-carboxamide

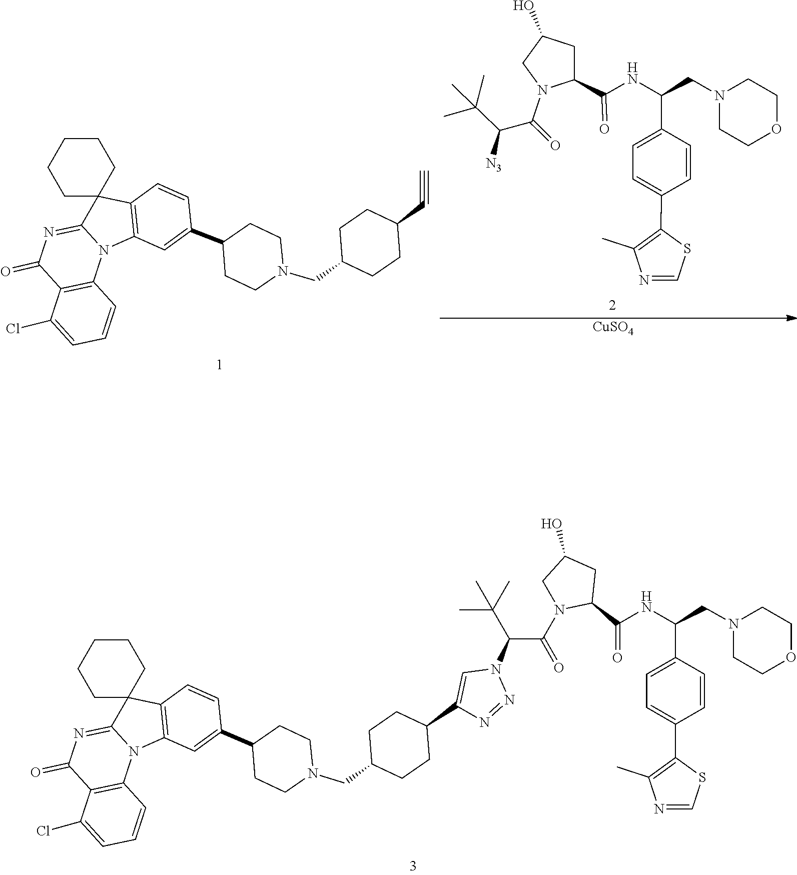

Compound A70 was synthesized using the similar procedures as used in the synthesis of Compound A49. ESI MS m/z: 1095.55 [M+H]⁺.

### Synthesis of Compound 4A, 4B, 4C, 4D, 4E, and 4F.

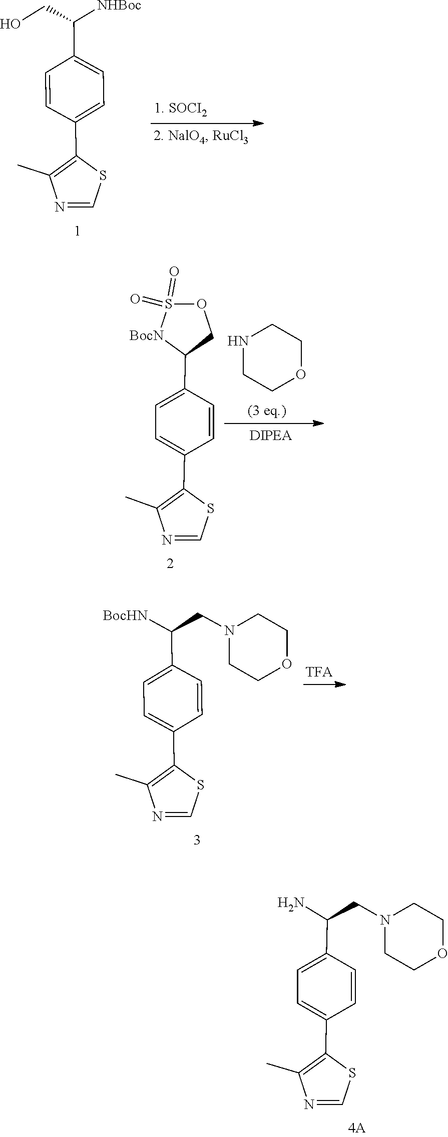

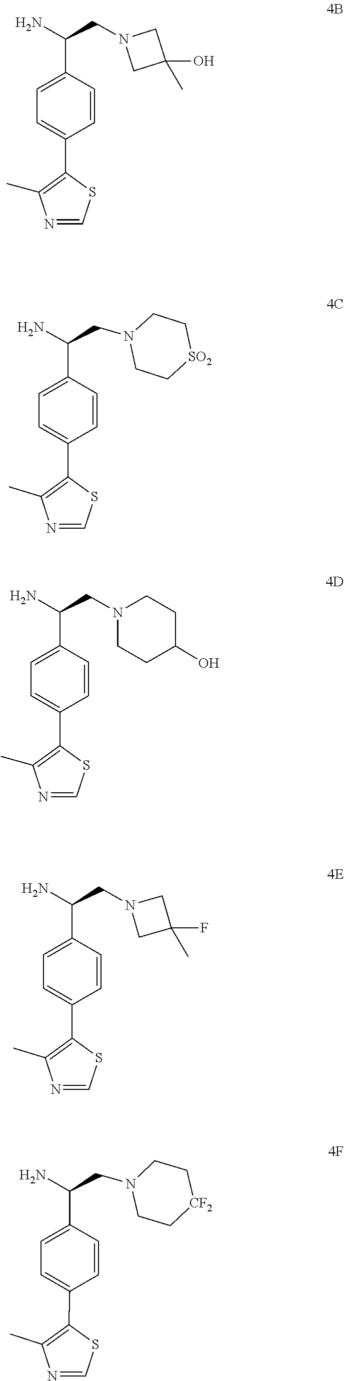 [SMILES: Cc1ncsc1-c1ccc([C@@H](N)CN2CC(C)(O)C2)cc1 ; Cc1ncsc1-c1ccc([C@@H](N)CN2CCS(=O)(=O)CC2)cc1 ; Cc1ncsc1-c1ccc([C@@H](N)CN2CCC(O)CC2)cc1 ; Cc1ncsc1-c1ccc([C@@H](N)CN2CC(C)(F)C2)cc1 ; Cc1ncsc1-c1ccc([C@@H](N)CN2CCC(F)(F)CC2)cc1]

Preparation of 2. The 1 (2.2 g, 1.0 eq.) in CH₂C₁₂ (15 mL) was added dropwise to a stirred solution of SOCl₂ (1.1 eq.), NEt3 (2.2 eq.) and imidazole (4 eq.) in CH₂C₁₂ (15 mL) at −78° C. (CO₂ and EtOH) and the resulting solution was stirred at −78° C. for 3 h. The reaction mixture was warmed to room temperature, quenched by addition of water, phases separated, organic phase washed with brine, dried (Na₂SO₄) and concentrated in vacuo to give the crude cyclic sulfamidite. The crude cyclic sulfamidite was dissolved in MeCN (0.13 M) at rt, NaIO4 (1.6 eq) RuCl3 (0.5 mol %) and water (0.16 M) were added sequentially and the resulting solution was stirred until completion was indicated by TLC. The reaction mixture was diluted with water (equal volume to that used in reaction), extracted with Et2O, washed with brine, dried (Na₂SO₄) and concentrated in vacuo to give the crude product 2 (88% for two steps).

Preparation of 3. To a solution of 2 (47 mg, 0.12 mmol, 1 equiv.) in CH₃CN (1.2 mL) was added morpholine (31 mg, 0.36 mmol, 3 equiv.) and DIPEA (150 μL, 0.96 mmol, 8.0 equiv.). The mixture was stirred at rt for 3 h. After reaction, the solvent was removed, the residue was purified by silica gel column chromatography (elution solvents: ethyl acetate/hexane=1/1) to get pure product 3 (86%).

Preparation of 4A. To a solution of 3 (80 mg, 0.20 mmol, 1 equiv.) in DCM (3 mL), was added TFA (3 mL) and the mixture was stirred 30 min. After removing solvent, the crude product 4A was received and can be used in the next step directly. ESI MS m/z: 304.14 [M+H]⁺.

Compound 4B-F was synthesized using the similar procedures as used in the synthesis of Compound 4A

### Compound A71. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-2-(3-hydroxy-3-methylazetidin-1-yl)-1-(4-(4-methylthiazol-5-yl)phenyl)ethyl)pyrrolidine-2-carboxamide

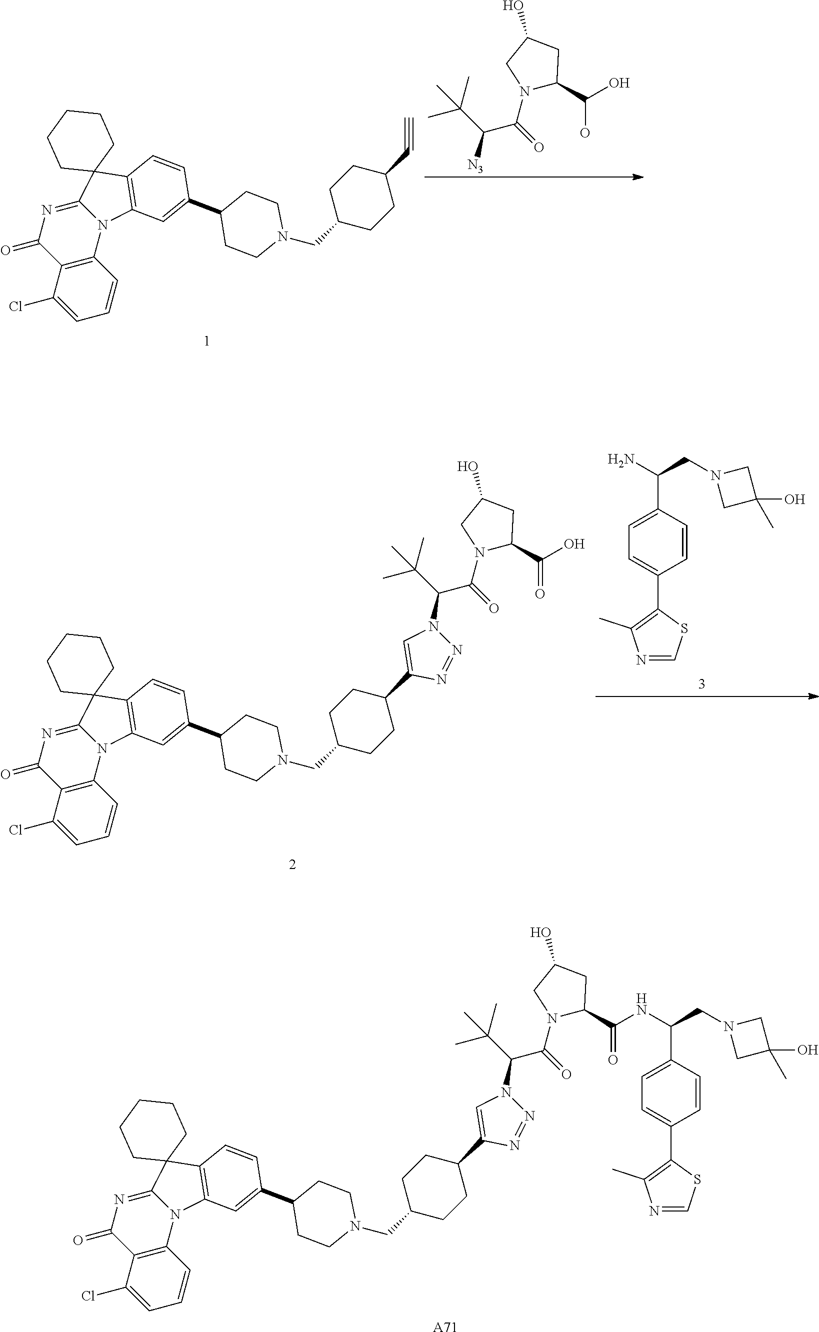

Compound A71 was synthesized using the similar procedures as used in the synthesis of Compound A67. ESI MS m/z: 1095.55 [M+H]⁺.

### Compound A72. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-N—((R)-2-(1,1-dioxidothiomorpholino)-1-(4-(4-methylthiazol-5-yl)phenyl)ethyl)-4-hydroxypyrrolidine-2-carboxamide

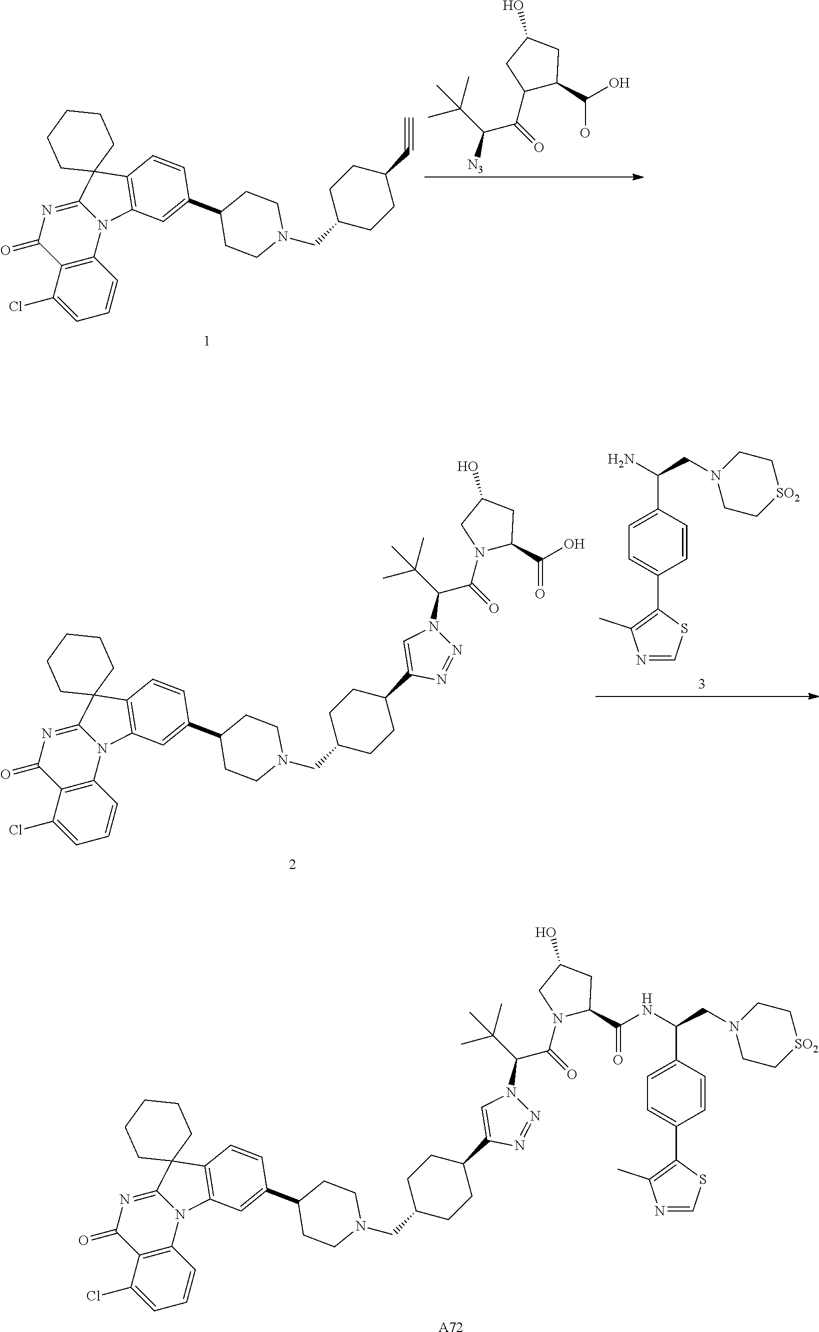

Compound A72 was synthesized using the similar procedures as used in the synthesis of Compound A67. ESI MS m/z: 1143.55 [M+H]⁺.

### Compound A73. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-2-(4-hydroxypiperidin-1-yl)-1-(4-(4-methylthiazol-5-yl)phenyl)ethyl)pyrrolidine-2-carboxamide

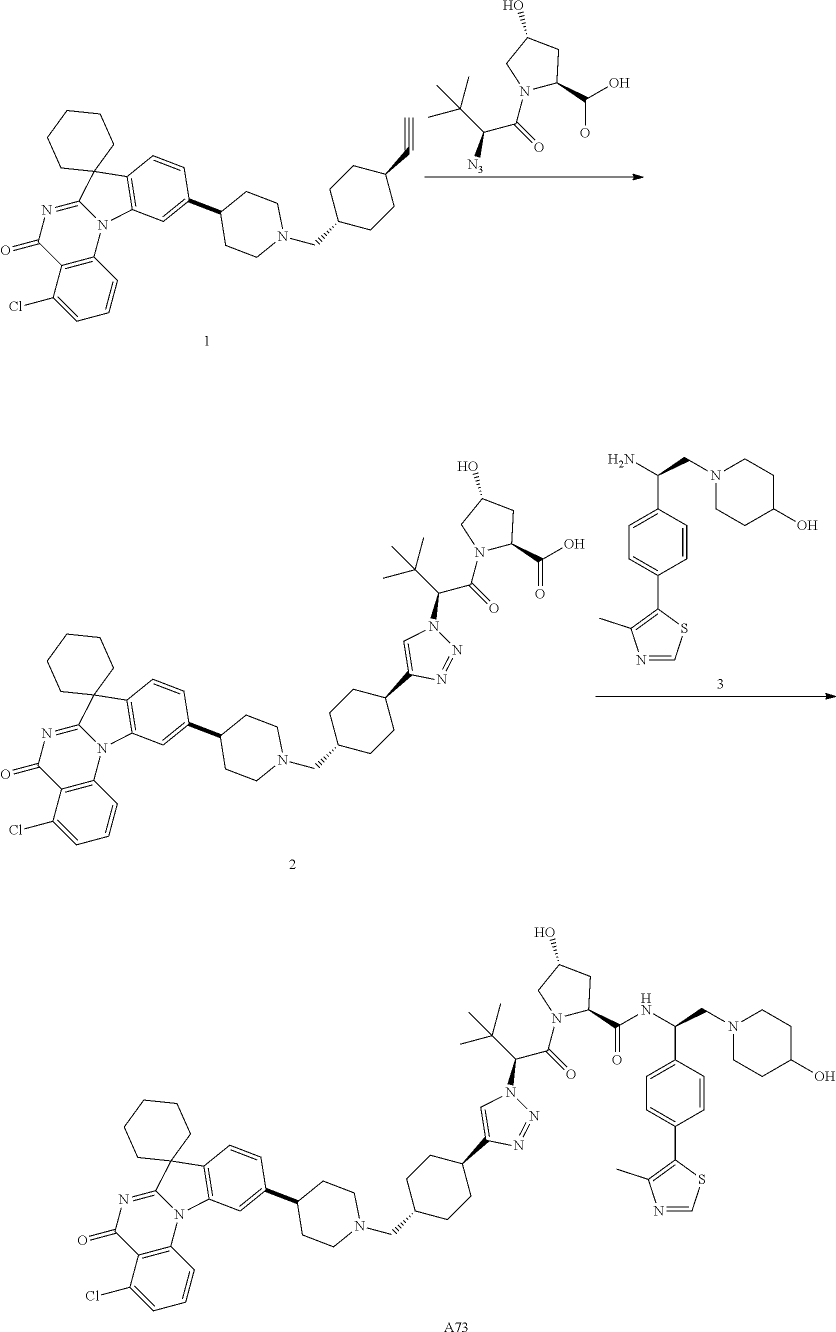

Compound A73 was synthesized using the similar procedures as used in the synthesis of Compound A67. ESI MS m/z: 1109.75 [M+H]⁺.

### Compound A74. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-N—((R)-2-(3-fluoro-3-methylazetidin-1-yl)-1-(4-(4-methylthiazol-5-yl)phenyl)ethyl)-4-hydroxypyrrolidine-2-carboxamide

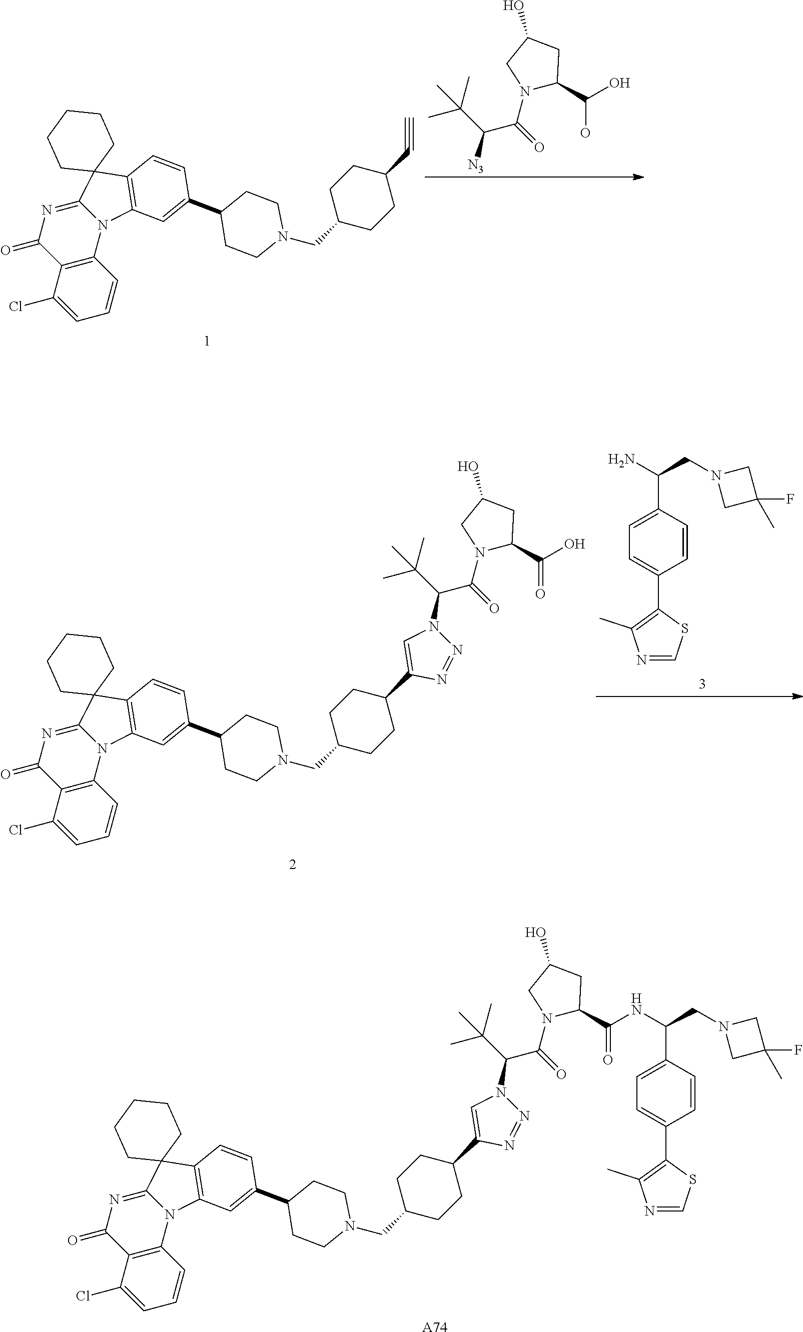

Compound A74 was synthesized using the similar procedures as used in the synthesis of Compound A67. ESI MS m/z: 1097.87 [M+H]⁺.

### Compound A75. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-N—((R)-2-(4,4-difluoropiperidin-1-yl)-1-(4-(4-methylthiazol-5-yl)phenyl)ethyl)-4-hydroxypyrrolidine-2-carboxamide

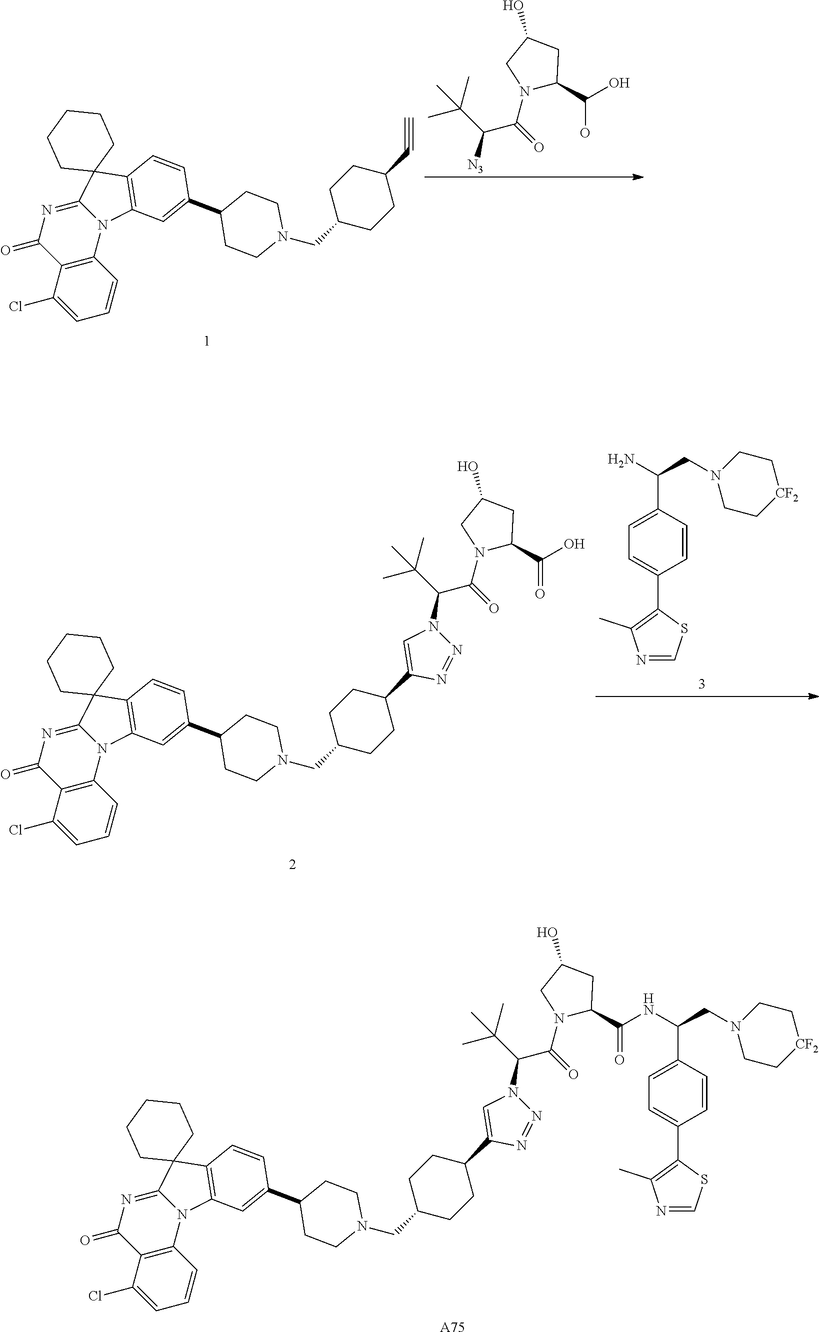

Compound A75 was synthesized using the similar procedures as used in the synthesis of Compound A67. ESI MS m/z: 1129.78 [M+H]⁺.

### Compound A76. (2S,4R)-1-((S)-2-(4-((1r,4S)-4-((4-(4′-bromo-5′-oxo-5′H-spiro[cyclohexane-1,7′-pyrido[2′,3′: 4,5]pyrrolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

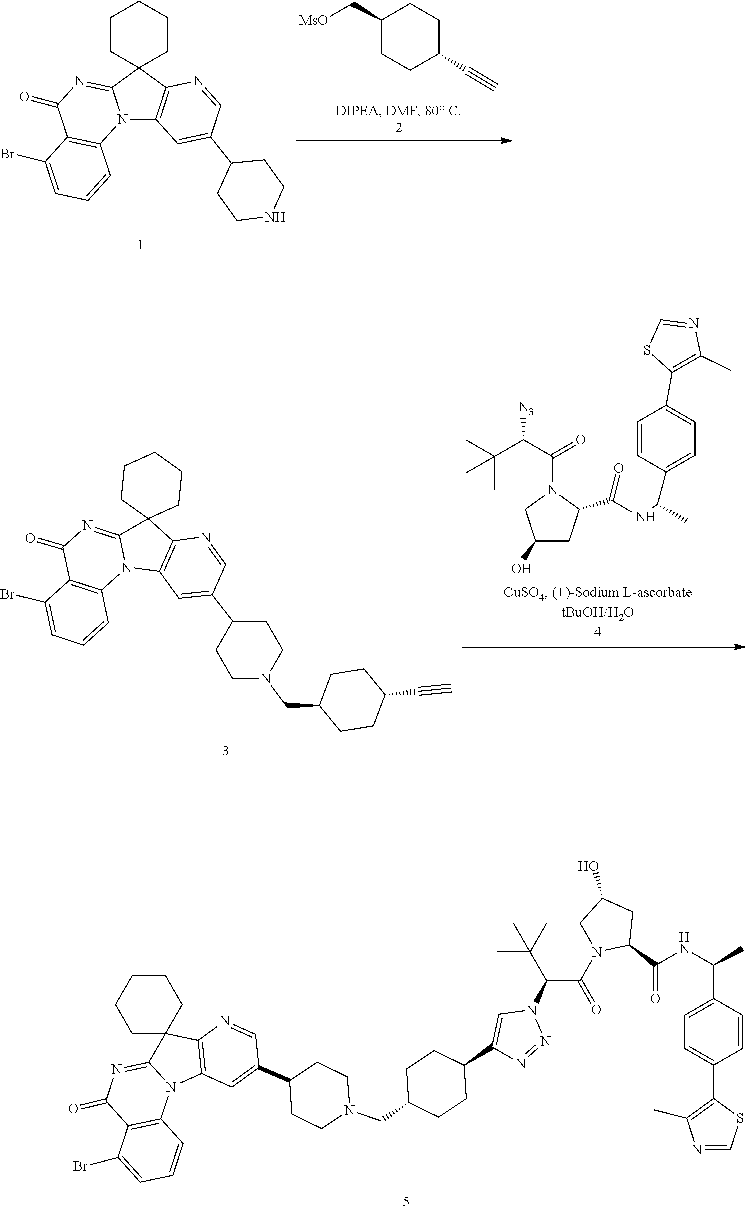

Compound A76 was synthesized using the similar procedures as used in the synthesis of Compound A49. ESI MS m/z: 1055.65 [M+H]⁺.

### Compound A77. (2S,4R)-1-((S)-2-(4-((1r,4S)-4-((4-(4′-chloro-5′-oxo-5′H-spiro[cyclohexane-1,7′-pyrido[3′,2′: 4,5]pyrrolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-1-(4-(4-methylthiazol-5-yl)phenyl)-2-morpholinoethyl)pyrrolidine-2-carboxamide

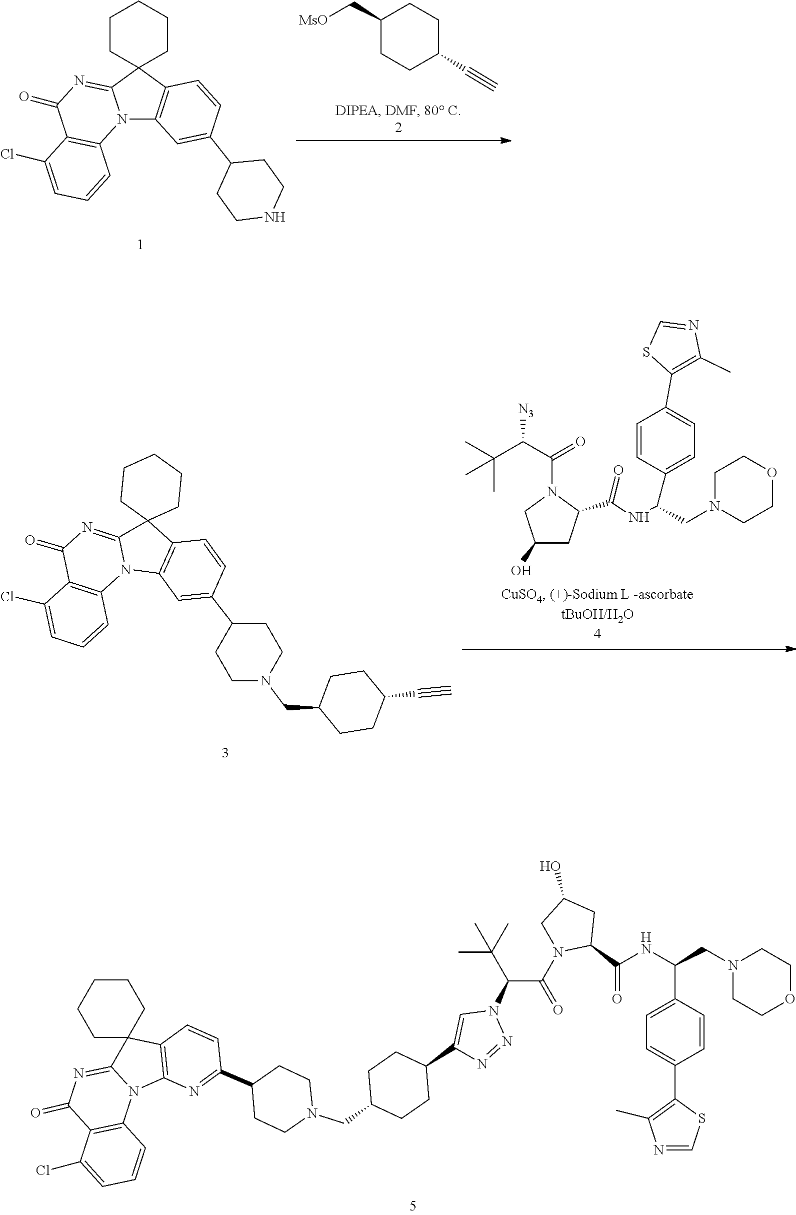

Compound A77 was synthesized using the similar procedures as used in the synthesis of Compound A49. ESI MS m/z: 1096.76 [M+H]⁺.

### Compound A78. (2S,4R)-1-((S)-2-(4-((1r,4S)-4-((4-(4′-chloro-5′-oxo-5′H-spiro[cyclohexane-1,7′-pyrido[3′,2′: 4,5]pyrrolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

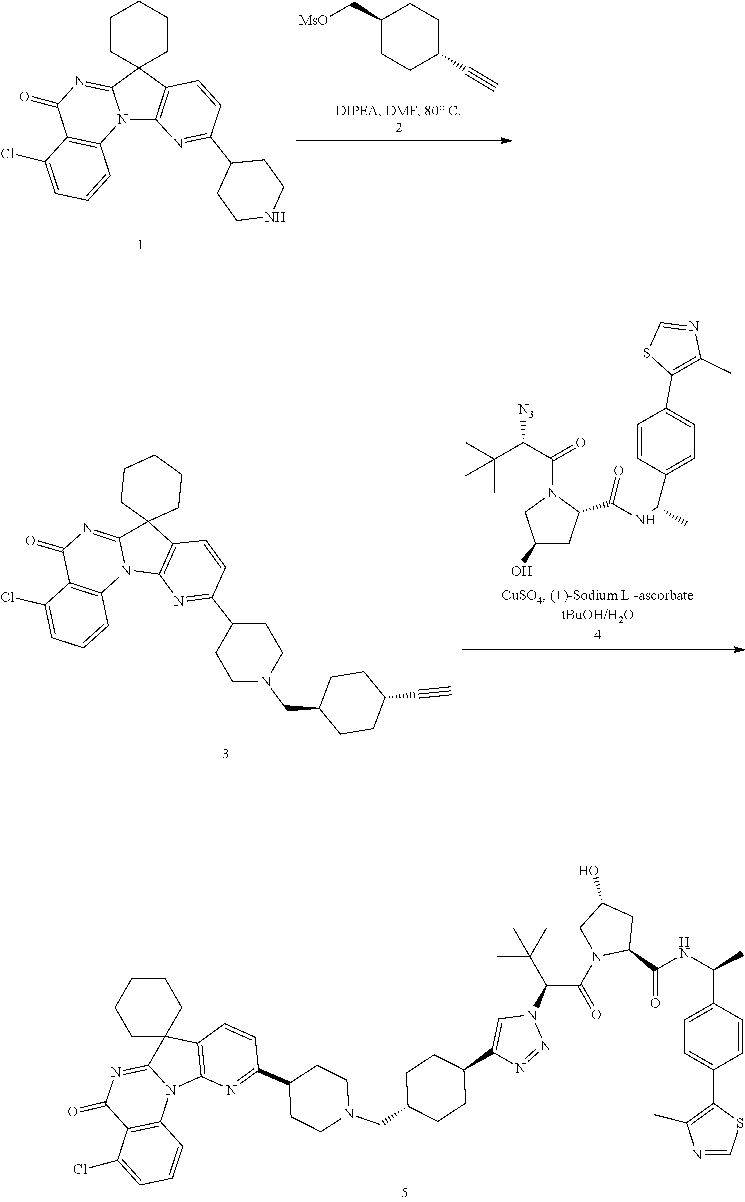

Compound A78 was synthesized using the similar procedures as used in the synthesis of Compound A49. ESI MS m/z: 1011.35 [M+H]⁺.

### Compound A79. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-N—((S)-3-(3-fluoro-3-methylazetidin-1-yl)-1-(4-(4-methylthiazol-5-yl)phenyl) propyl)-4-hydroxypyrrolidine-2-carboxamide

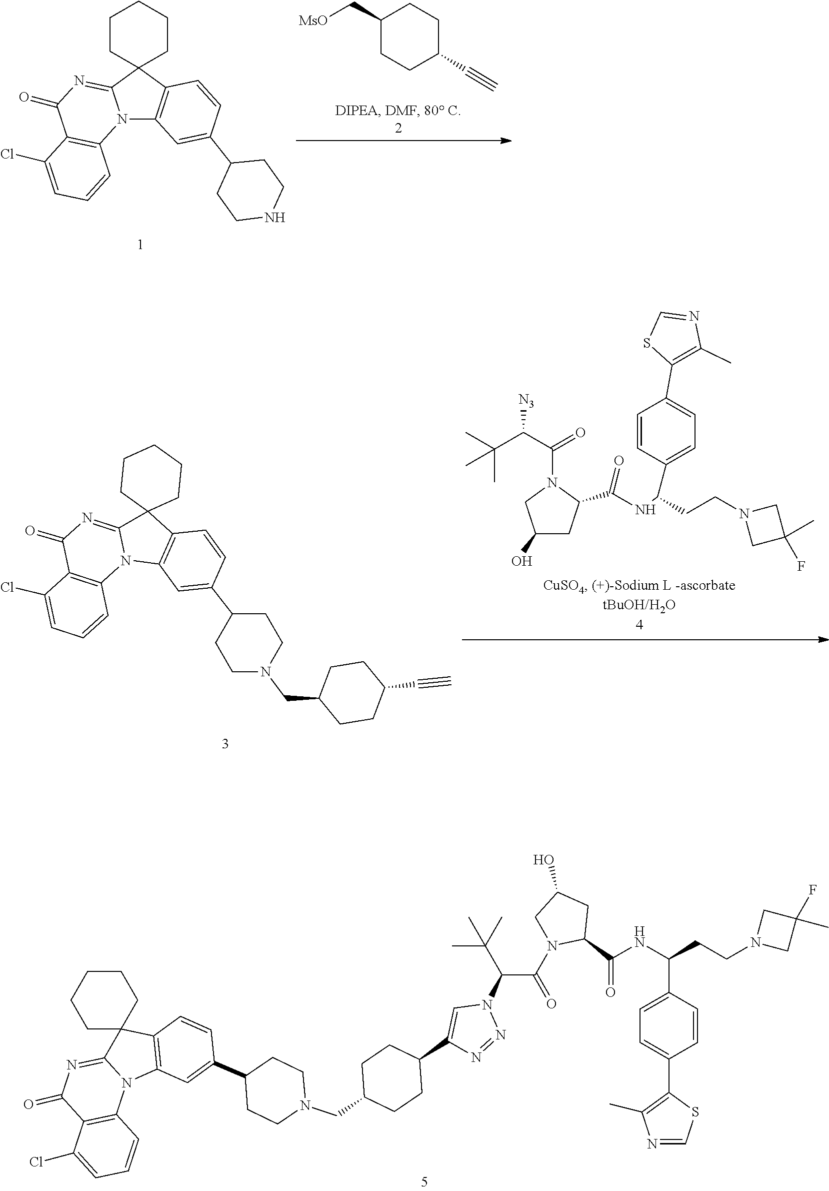

Compound A79 was synthesized using the similar procedures as used in the synthesis of Compound A71. ESI MS m/z: 1111.79 [M+H]⁺.

### Compound A80. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((S)-3-(3-hydroxy-3-methylazetidin-1-yl)-1-(4-(4-methylthiazol-5-yl)phenyl) propyl)pyrrolidine-2-carboxamide

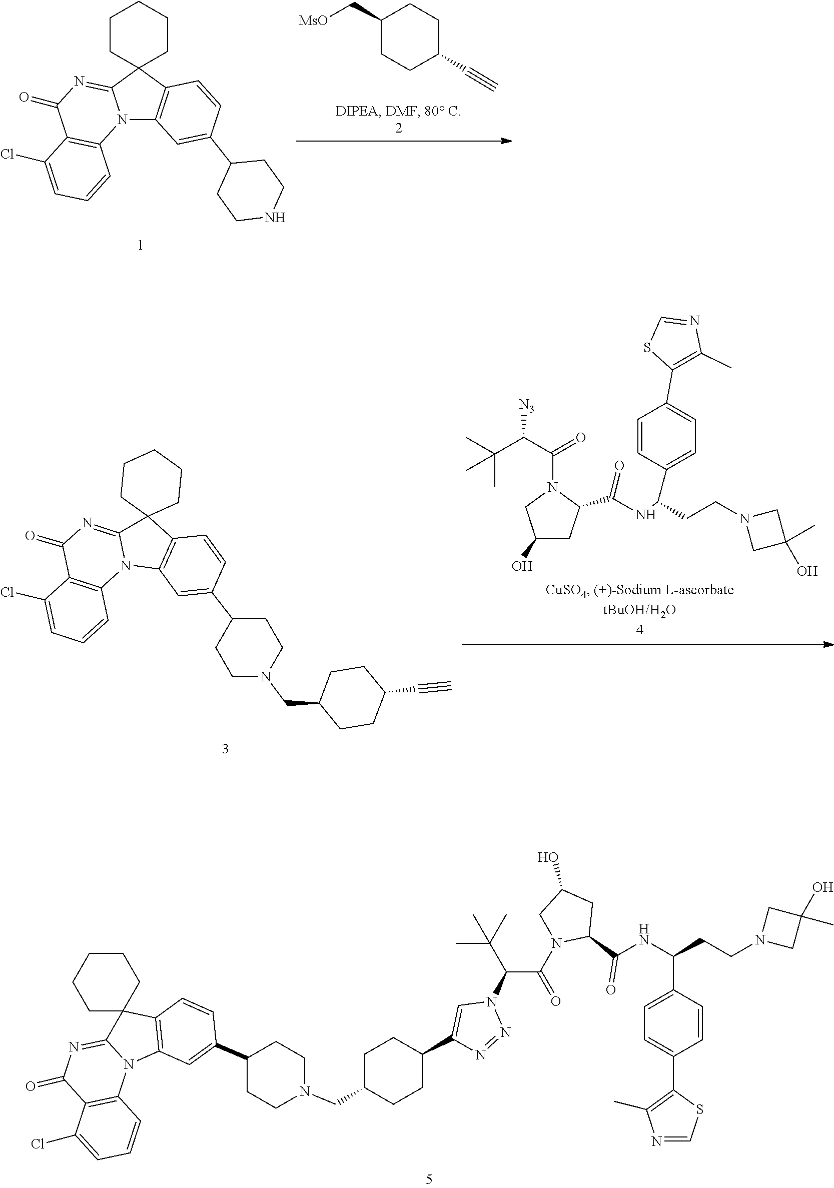

Compound A80 was synthesized using the similar procedures as used in the synthesis of Compound A71. ESI MS m/z: 1109.42 [M+H]⁺.

### Compound A81. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((S)-1-(4-(4-methylthiazol-5-yl)phenyl)-3-morpholinopropyl)pyrrolidine-2-carboxamide

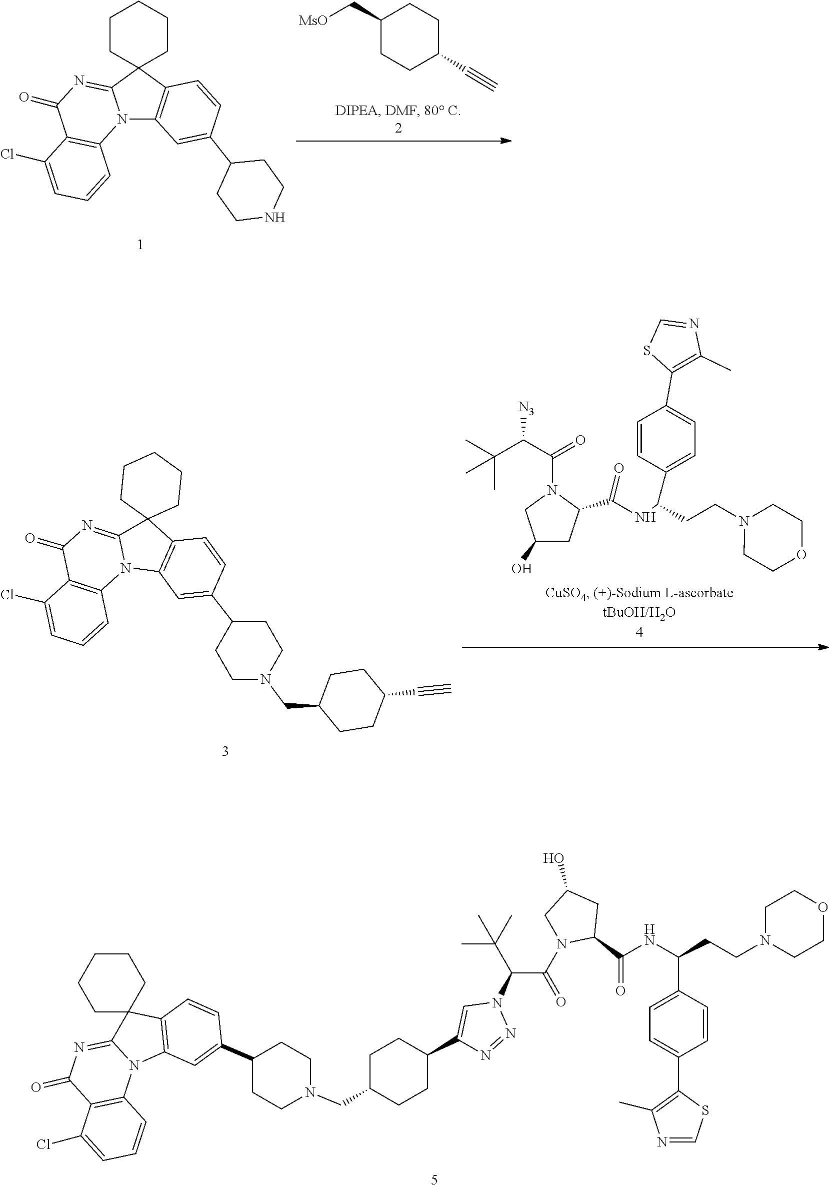

Compound A81 was synthesized using the similar procedures as used in the synthesis of Compound A71. ESI MS m/z: 1109.66 [M+H]⁺.

### Compound A82. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((S)-3-(2-(2-methoxyethoxy) ethoxy)-1-(4-(4-methylthiazol-5-yl)phenyl) propyl)pyrrolidine-2-carboxamide

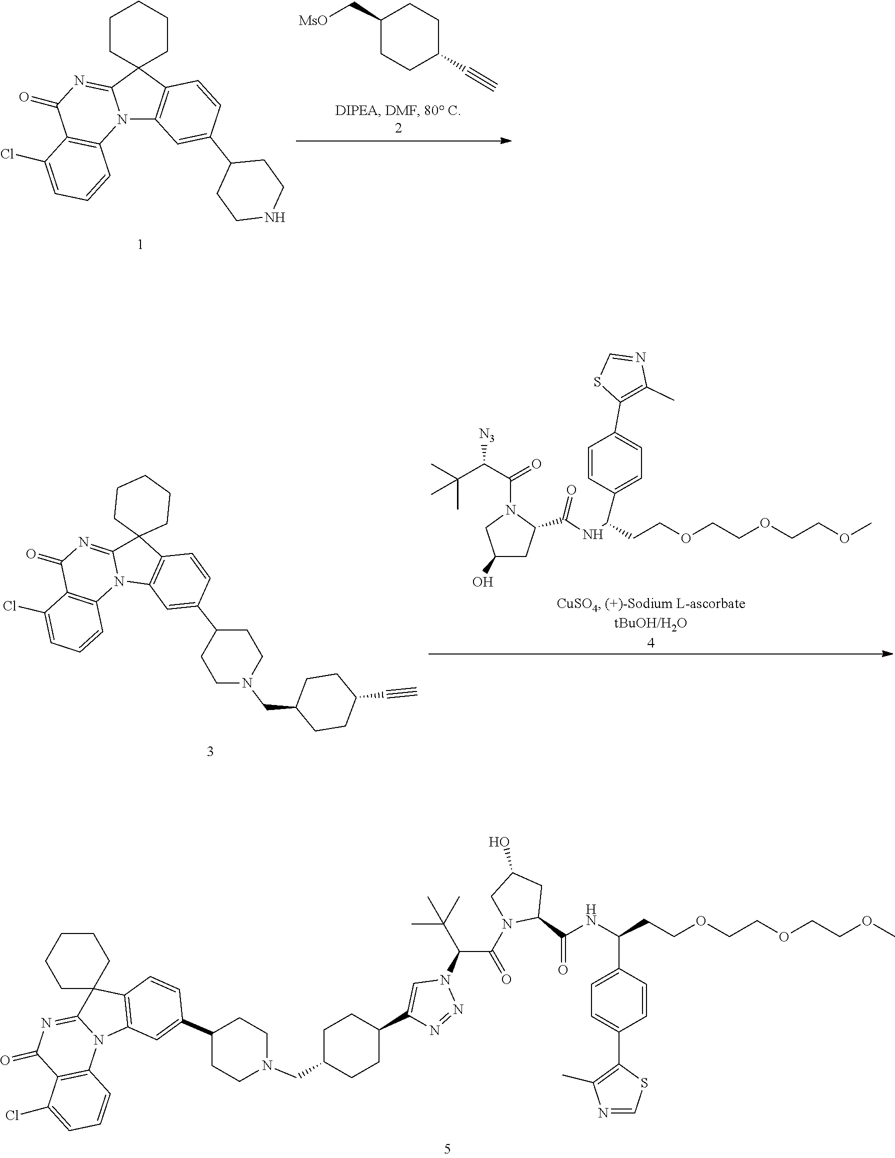

Compound A82 was synthesized using the similar procedures as used in the synthesis of Compound A71. ESI MS m/z: 1142.78 [M+H]⁺.

### Compound A83. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N-(®-1-(4-(4-methylthiazol-5-yl)phenyl)-2-(4-(trifluoromethyl)piperidin-1-yl)ethyl)pyrrolidine-2-carboxamide

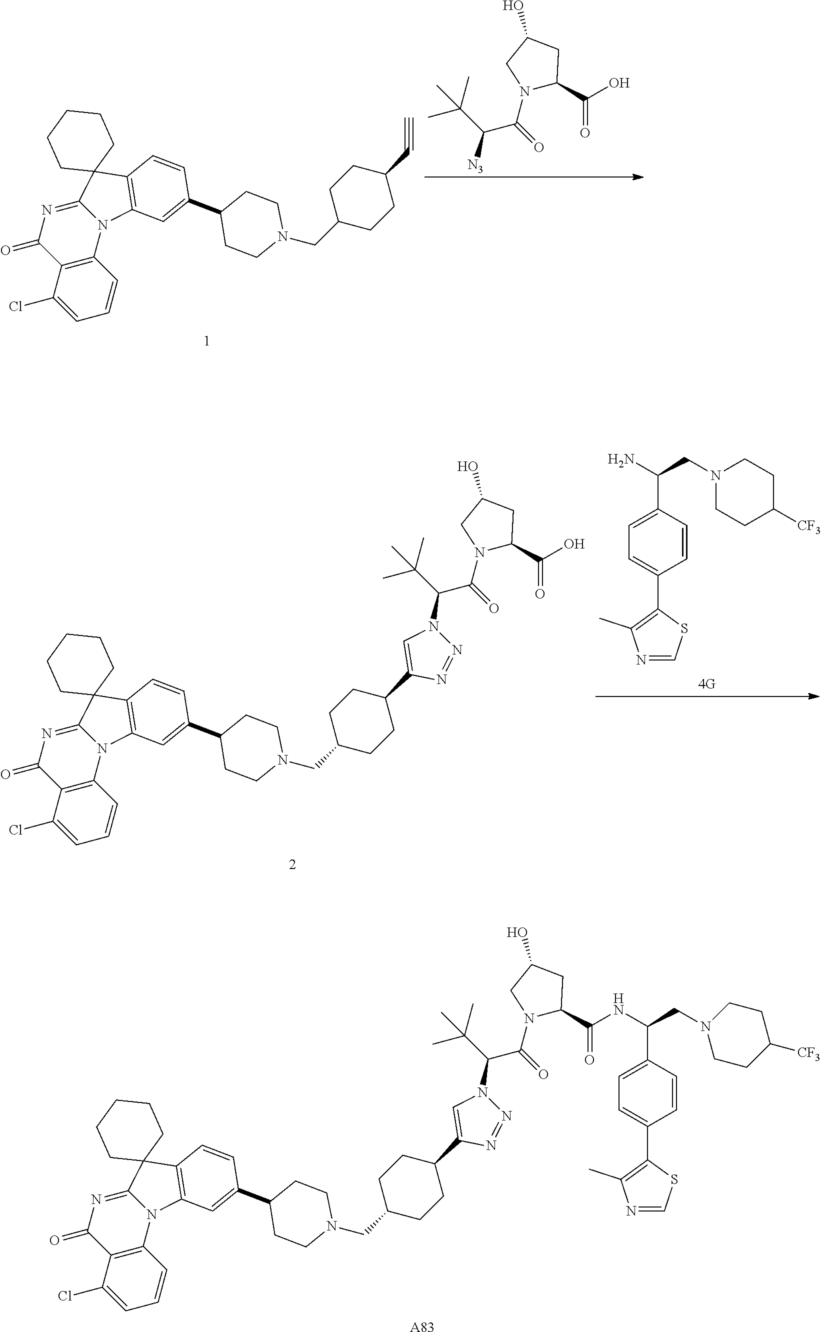

Compound A83 was synthesized using the similar procedures as used in the synthesis of Compound A67. ESI MS m/z: 1161.67 [M+H]⁺.

### Compound A84. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-N—((R)-2-(4-(difluoromethyl)piperidin-1-yl)-1-(4-(4-methylthiazol-5-yl)phenyl)ethyl)-4-hydroxypyrrolidine-2-carboxamide

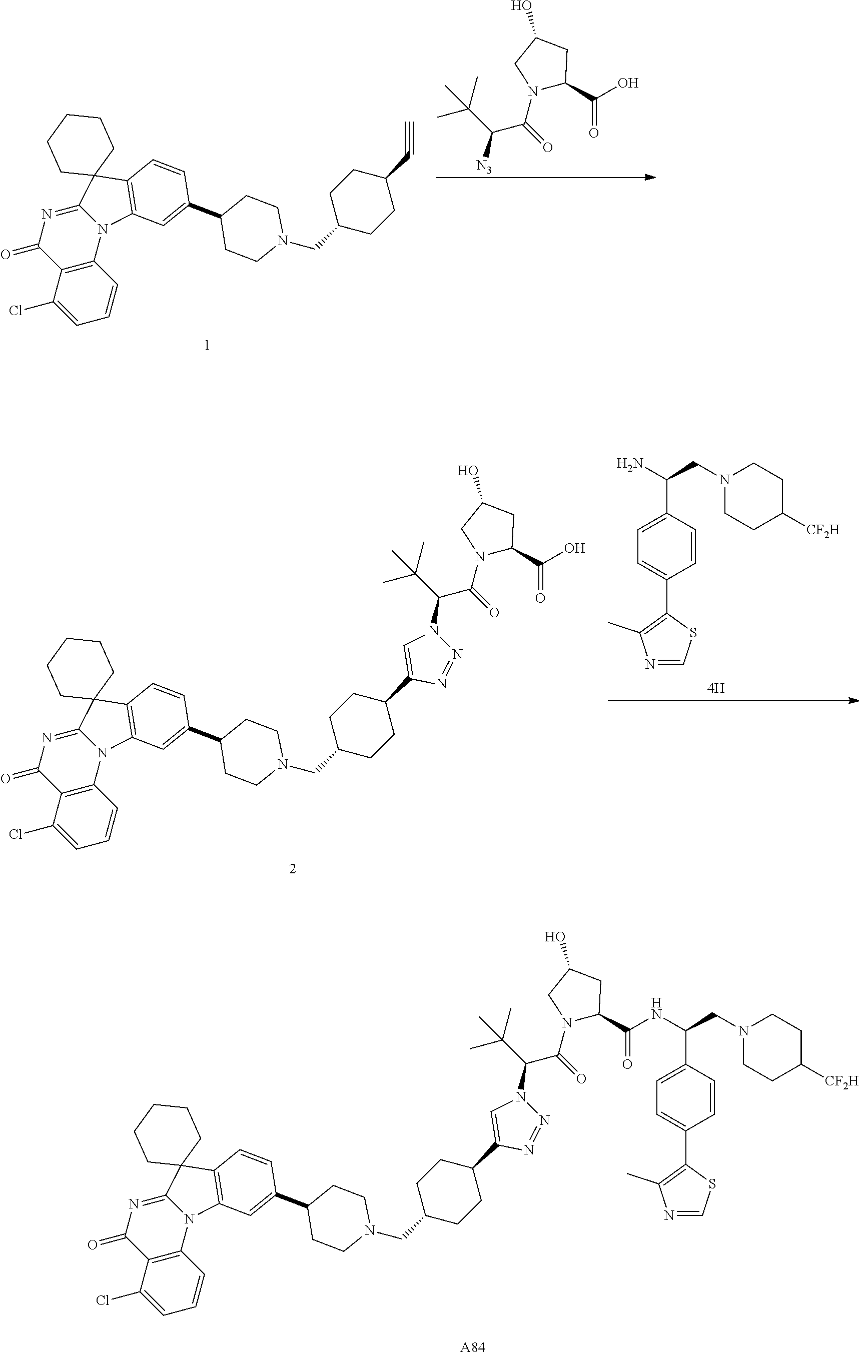

Compound A84 was synthesized using the similar procedures as used in the synthesis of Compound A67. ESI MS m/z: 1143.57 [M+H]⁺.

### Compound A85. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-2-(4-methylpiperazin-1-yl)-1-(4-(4-methylthiazol-5-yl)phenyl)ethyl)pyrrolidine-2-carboxamide

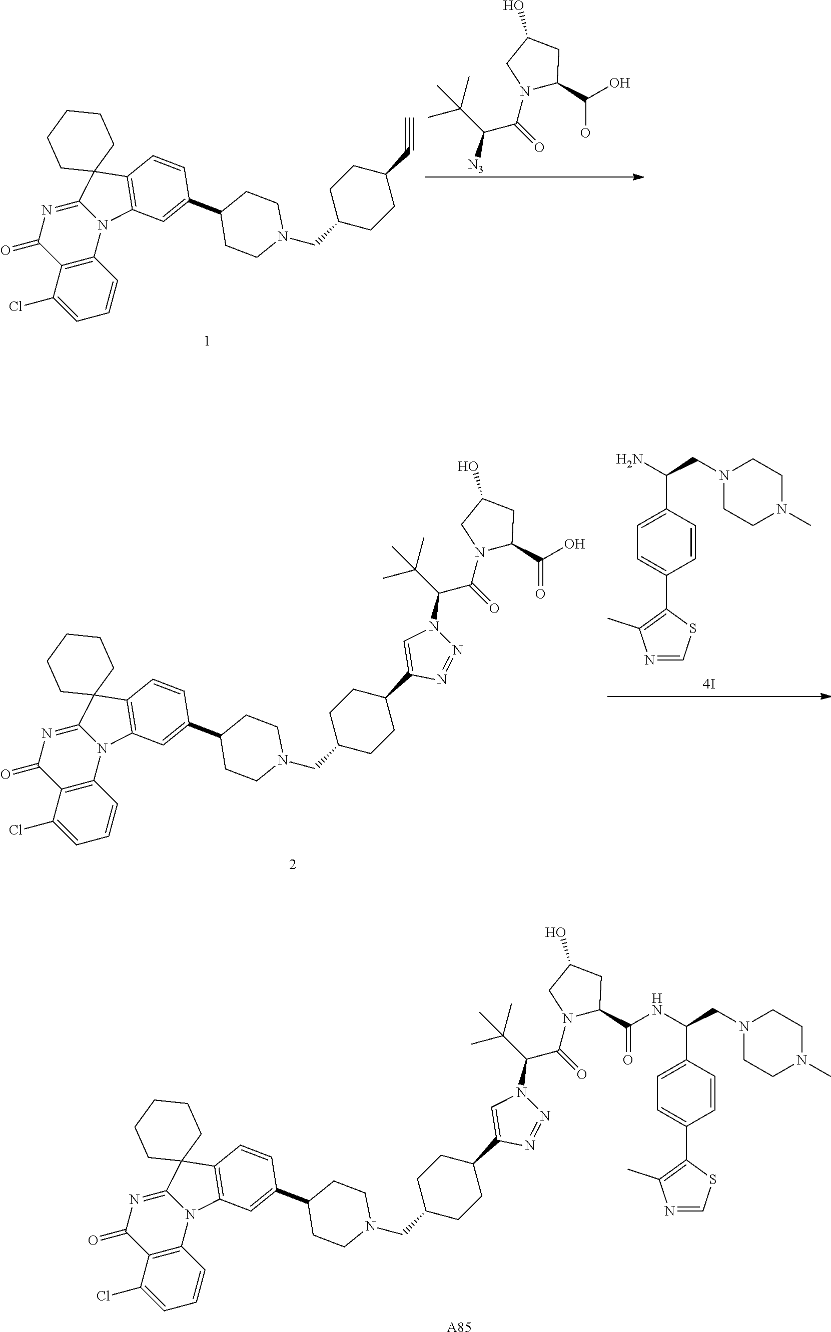

Compound A85 was synthesized using the similar procedures as used in the synthesis of Compound A67. ESI MS m/z: 1143.57 [M+H]⁺.

### Compound A86. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N-(®-2-(2-methoxyethoxy)-1-(4-(4-methylthiazol-5-yl)phenyl)ethyl)pyrrolidine-2-carboxamide

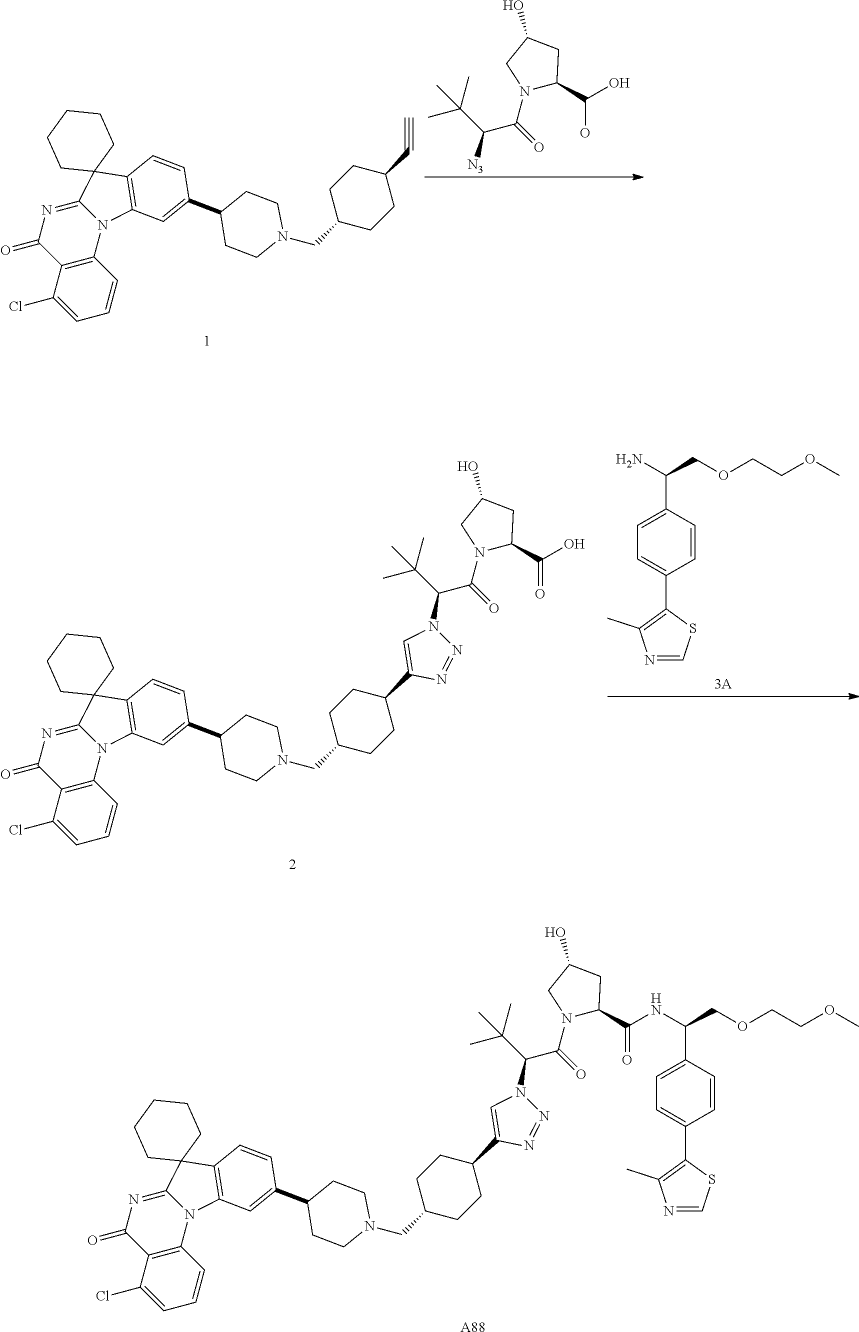

Compound A86 was synthesized using the similar procedures as used in the synthesis of Compound A67. ESI MS m/z: 1123.88 [M+H]⁺.

### Compound A87. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N-(®-2-(2-(2-methoxyethoxy) ethoxy)-1-(4-(4-methylthiazol-5-yl)phenyl)ethyl)pyrrolidine-2-carboxamide

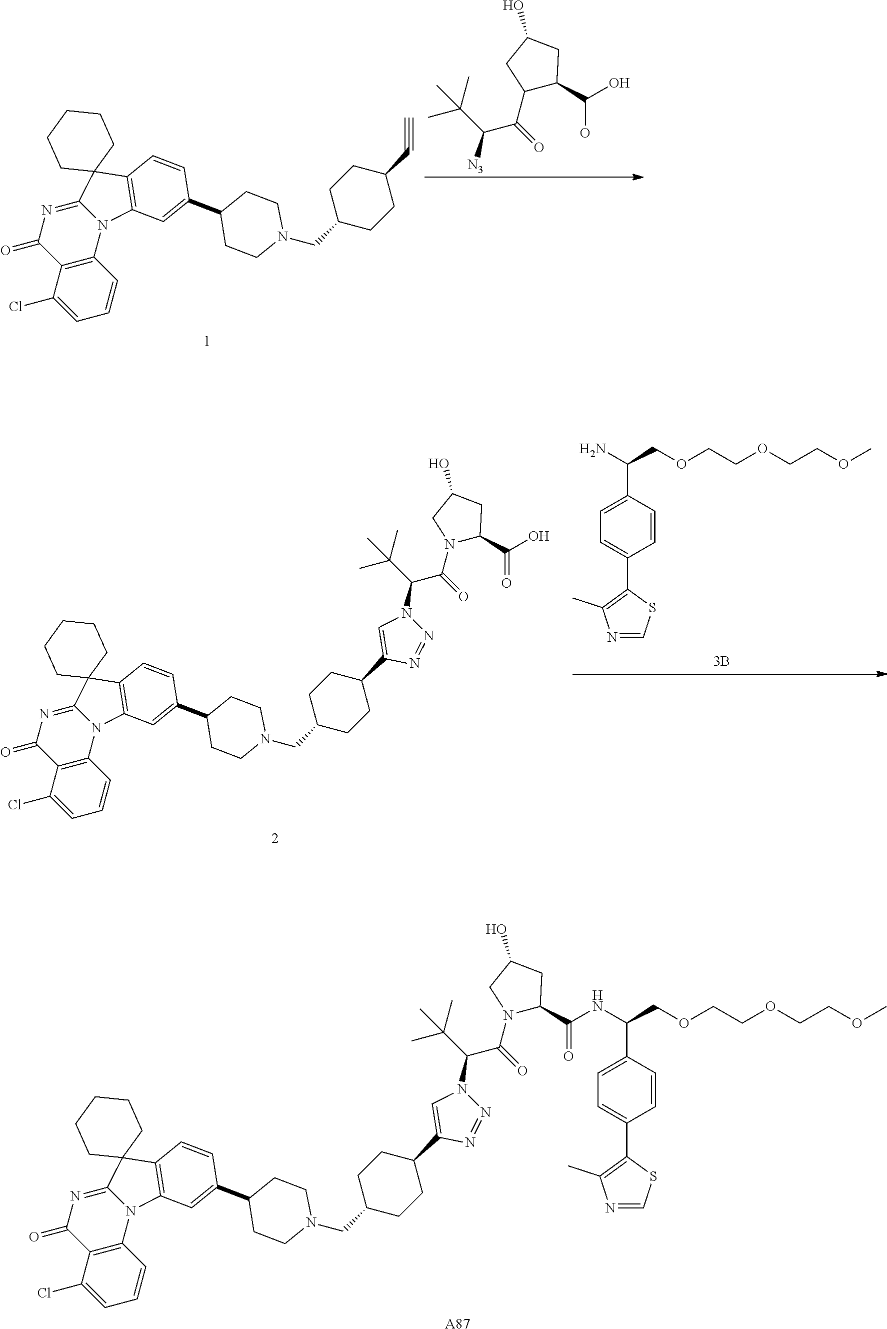

Compound A87 was synthesized using the similar procedures as used in the synthesis of Compound A67. ESI MS m/z: 1128.55 [M+H]⁺.

### Compound A88. (2S,4R)-1-((S)-2-(4-((1r,3S)-3-((7-(4′-chloro-5′-oxo-5′H-spiro[cyclohexane-1,7′-indolo[1,2-a]quinazolin]-10′-yl)-2-azaspiro[3.5]nonan-2-yl)methyl)cyclobutyl)-1H-1,2,3-triazol-1-yl)-3,3-dimethylbutanoyl)-4-hydroxy-N—((R)-1-(4-(4-methylthiazol-5-yl)phenyl)-2-morpholinoethyl)pyrrolidine-2-carboxamide

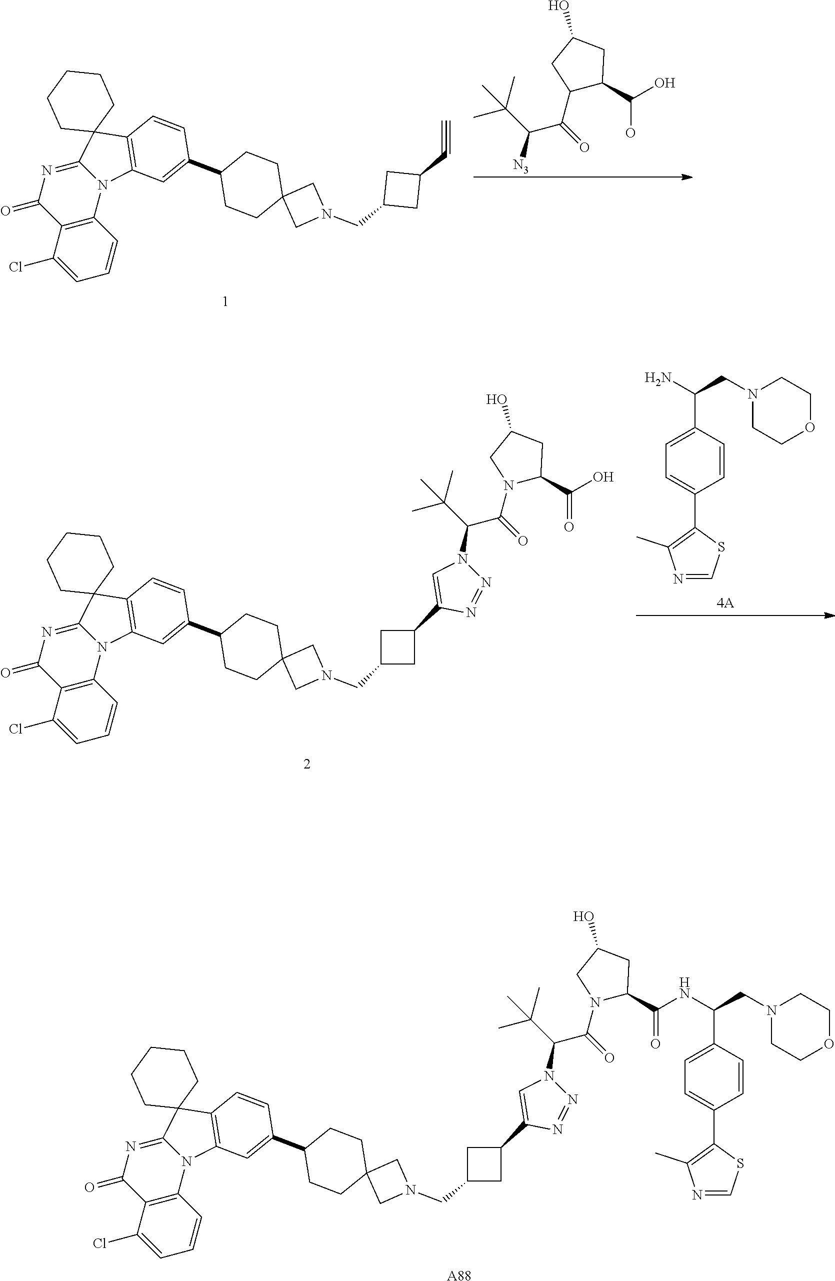

Compound A88 was synthesized using the similar procedures as used in the synthesis of Compound A67. ESI MS m/z: 1107.42 [M+H]⁺.

### Compound A89. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

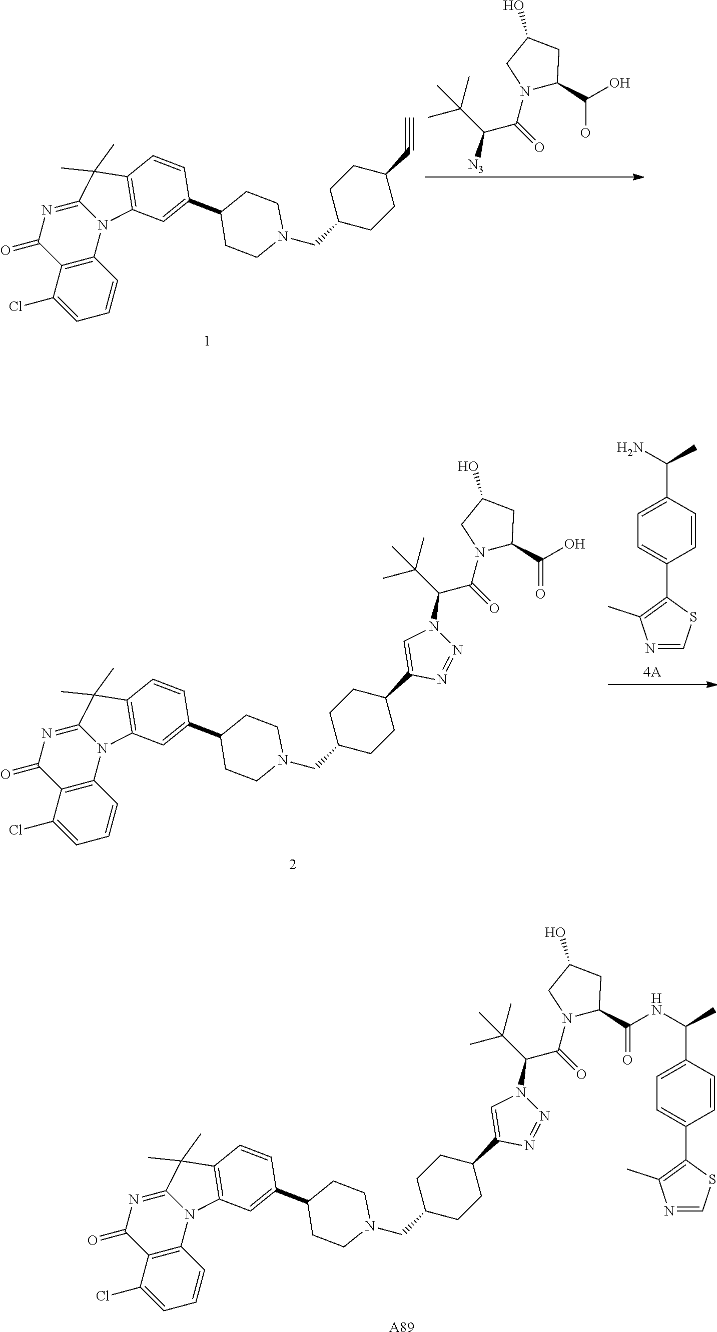

Compound A89 was synthesized using the similar procedures as used in the synthesis of Compound A67. ESI MS m/z: 970.51 [M+H]⁺.

### Compound A90. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-hydroxy-N—((R)-1-(4-(4-methylthiazol-5-yl)phenyl)-2-carboxamide

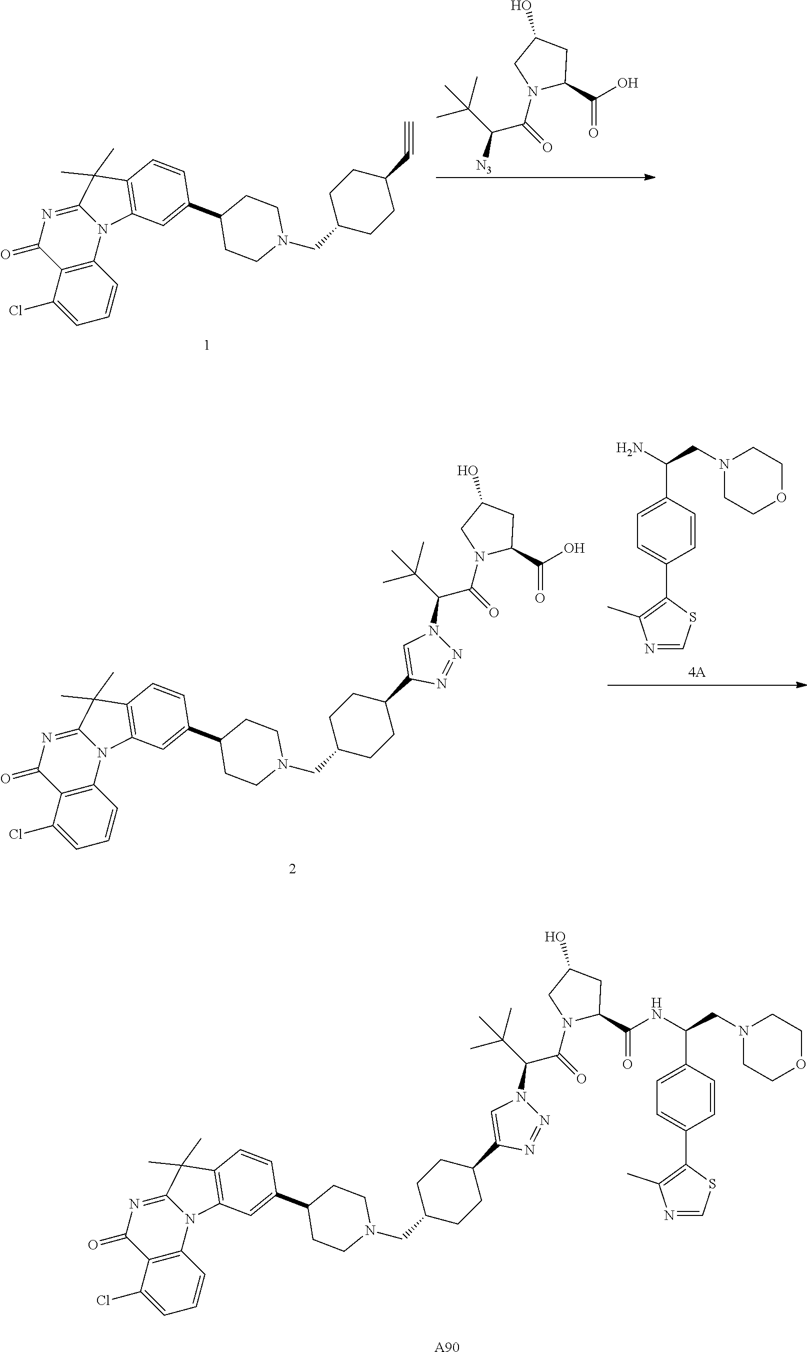

Compound A90 was synthesized using the similar procedures as used in the synthesis of Compound A67. ESI MS m/z: 1055.49 [M+H]⁺.

### Compound A91. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-hydroxy-N-(®-2-(2-methoxyethoxy)-1-(4-(4-methylthiazol-5-yl)phenyl)ethyl)pyrrolidine-2-carboxamide

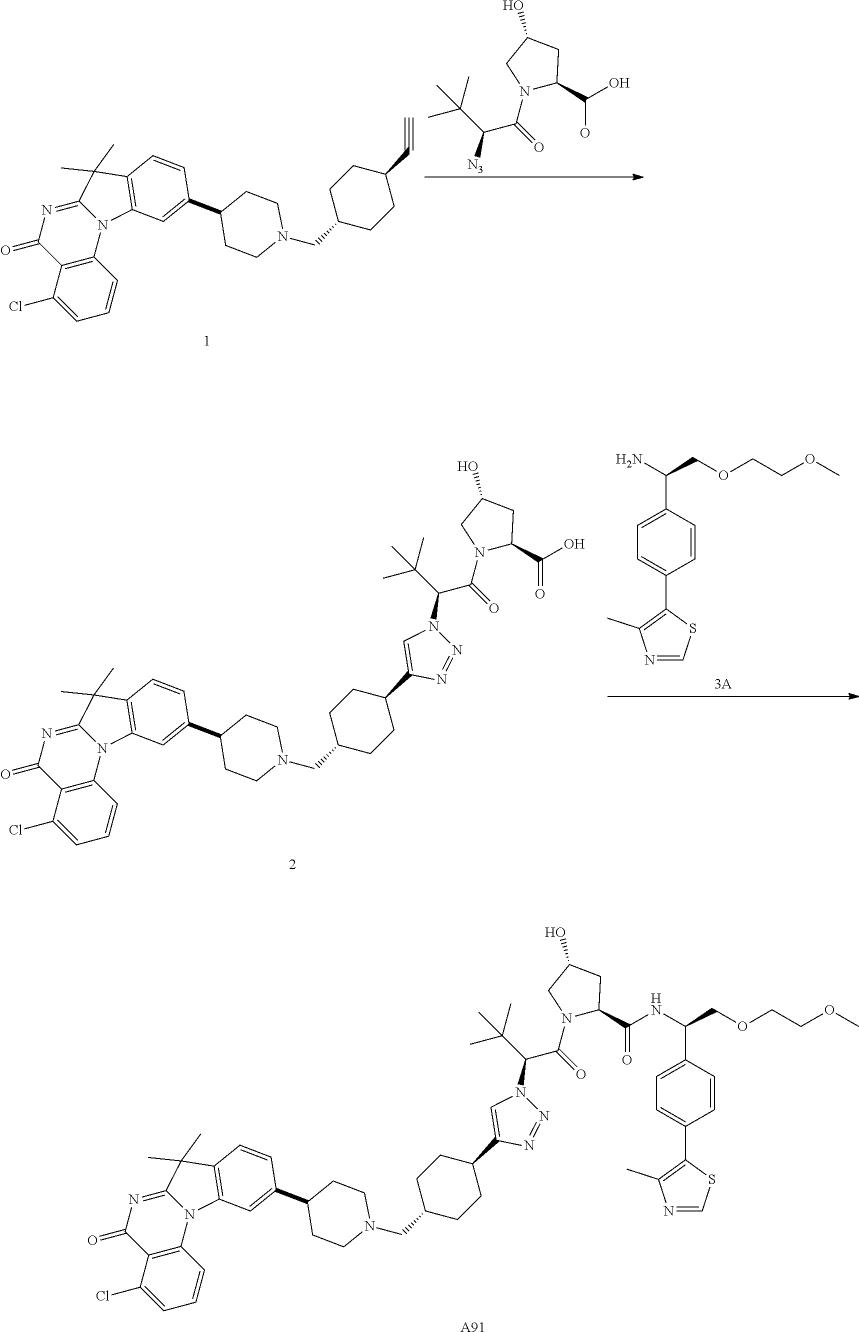

Compound A91 was synthesized using the similar procedures as used in the synthesis of Compound A67. ESI MS m/z: 1044.43 [M+H]⁺.

### Compound A92. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N-(®-2-(2-(2-methoxyethoxy) ethoxy)-1-(4-(4-methylthiazol-5-yl)phenyl)ethyl)pyrrolidine-2-carboxamide

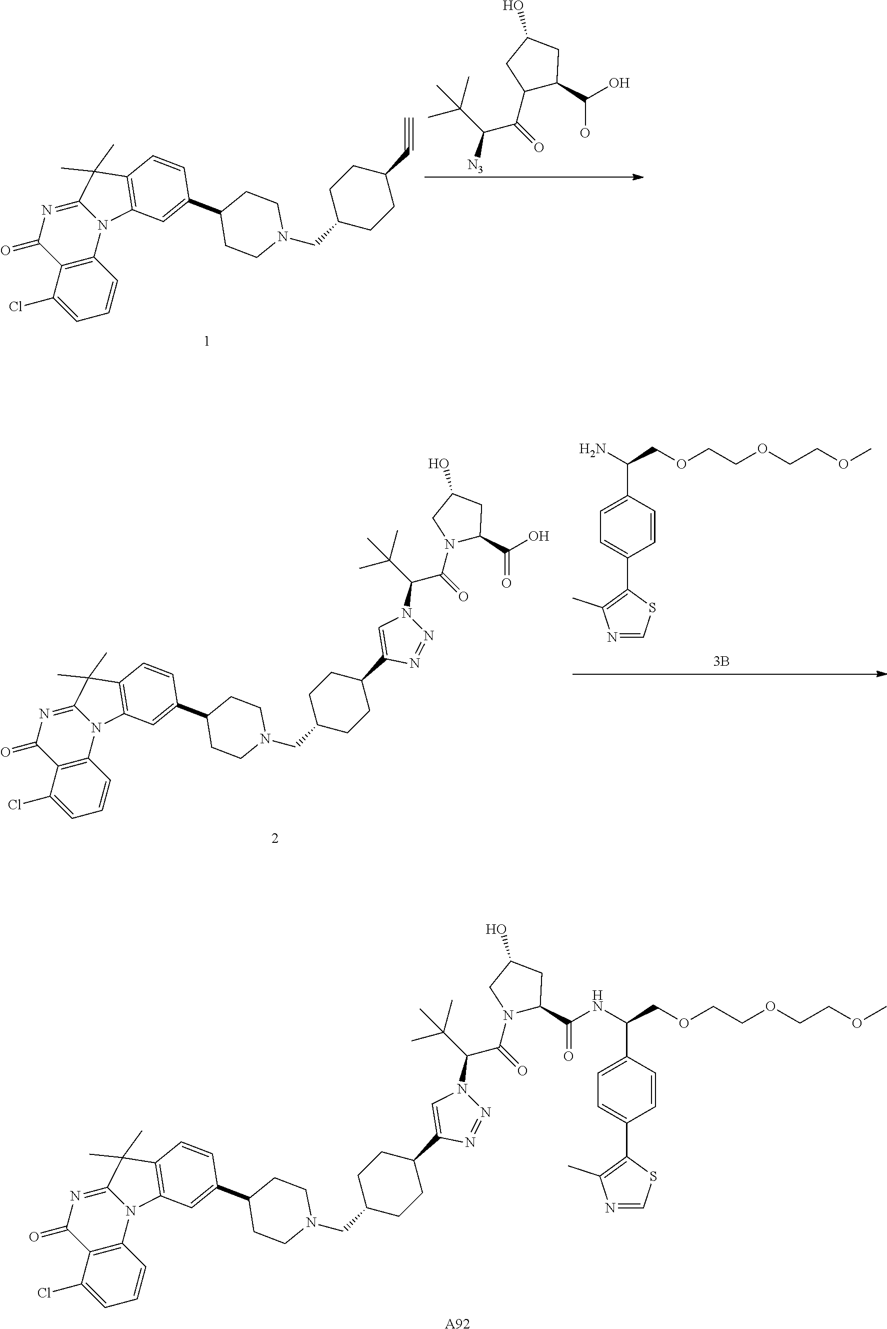

Compound A92 was synthesized using the similar procedures as used in the synthesis of Compound A67. ESI MS m/z: 1088.56 [M+H]⁺.

### Compound A93. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-N—((R)-2-(3-fluoro-3-methylazetidin-1-yl)-1-(4-(4-methylthiazol-5-yl)phenyl)ethyl)-4-hydroxypyrrolidine-2-carboxamide

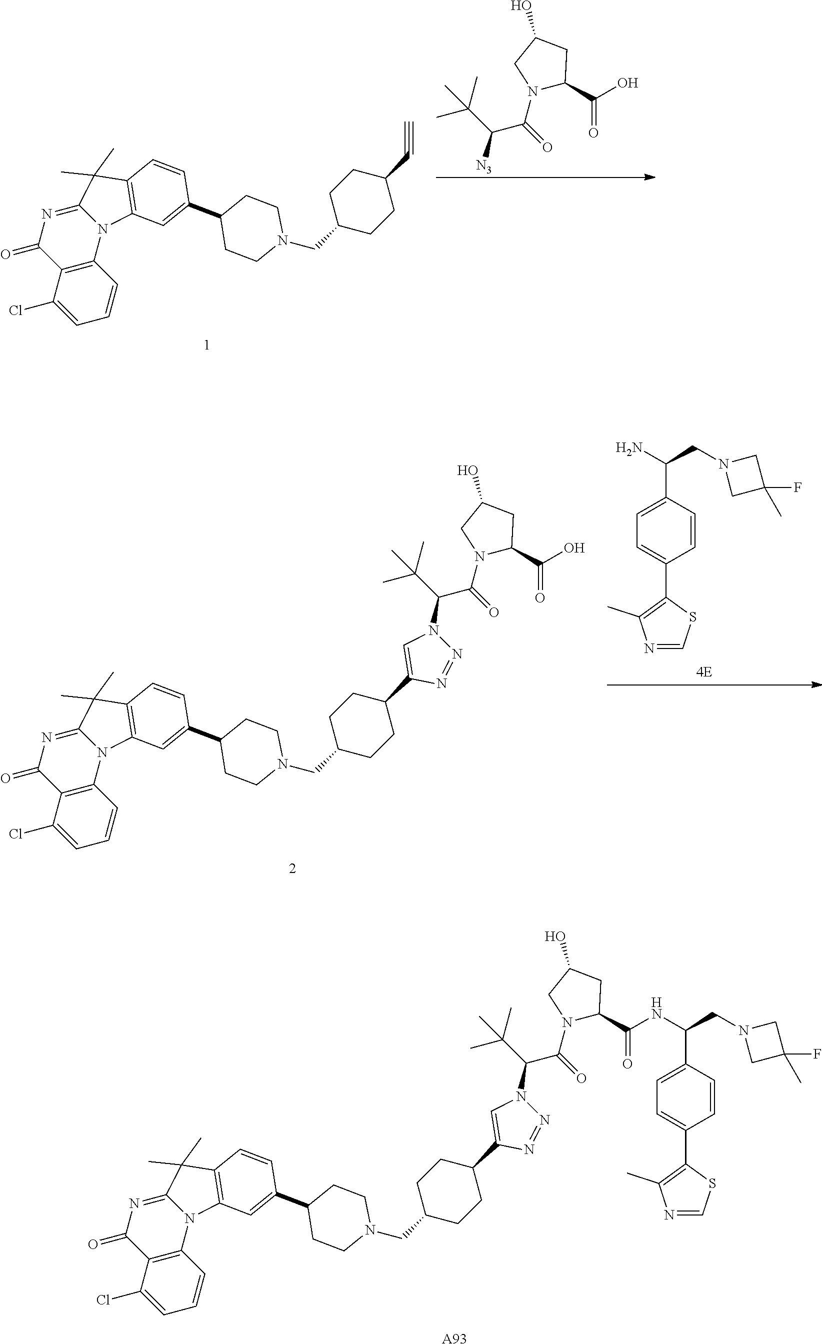

Compound A93 was synthesized using the similar procedures as used in the synthesis of Compound A67. ESI MS m/z: 1057.55 [M+H]⁺.

### Compound A94. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-N—((R)-2-(4,4-difluoropiperidin-1-yl)-1-(4-(4-methylthiazol-5-yl)phenyl)ethyl)-4-hydroxypyrrolidine-2-carboxamide

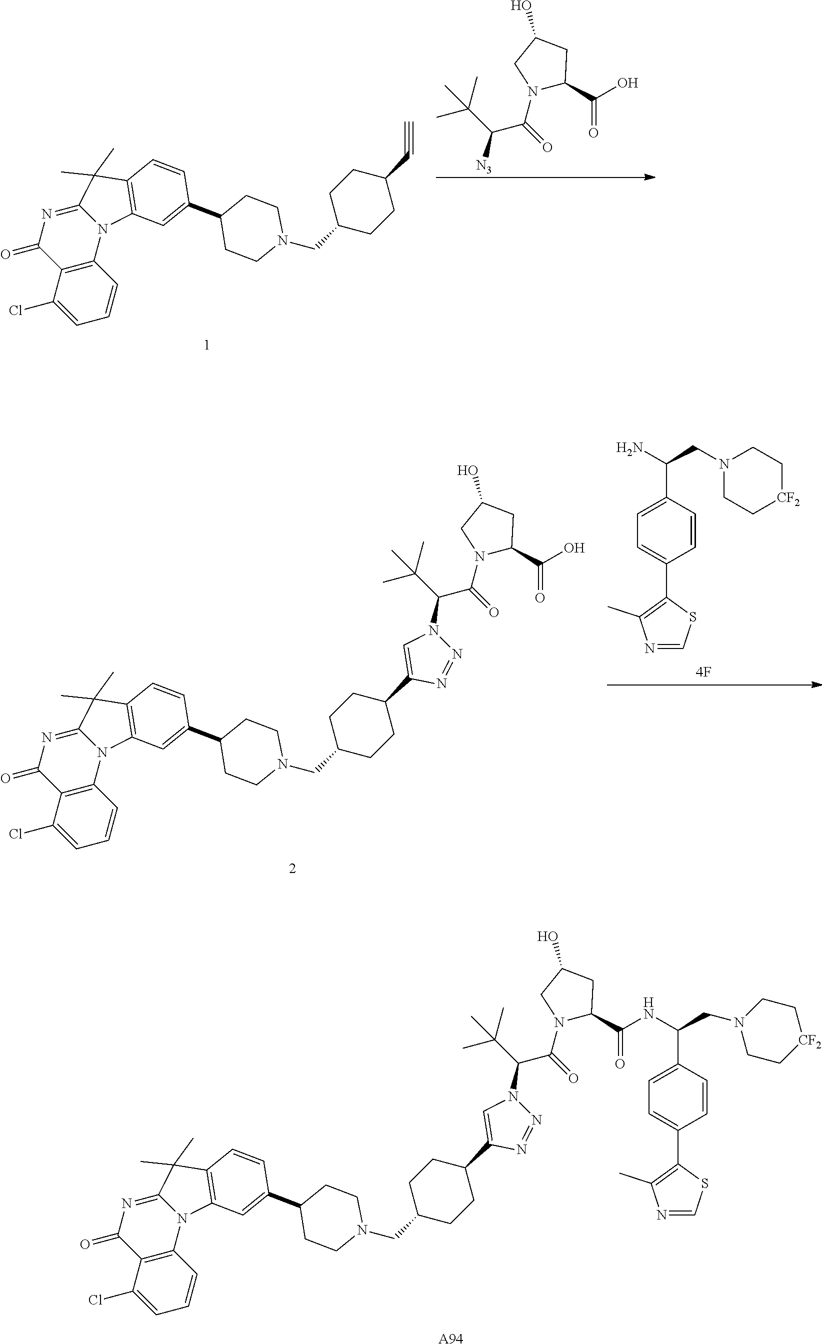

Compound A94 was synthesized using the similar procedures as used in the synthesis of Compound A67. ESI MS m/z: 1089.74 [M+H]⁺.

### Compound A95. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-hydroxy-N—((R)-2-(3-hydroxy-3-methylazetidin-1-yl)-1-(4-(4-methylthiazol-5-yl)phenyl)ethyl)pyrrolidine-2-carboxamide

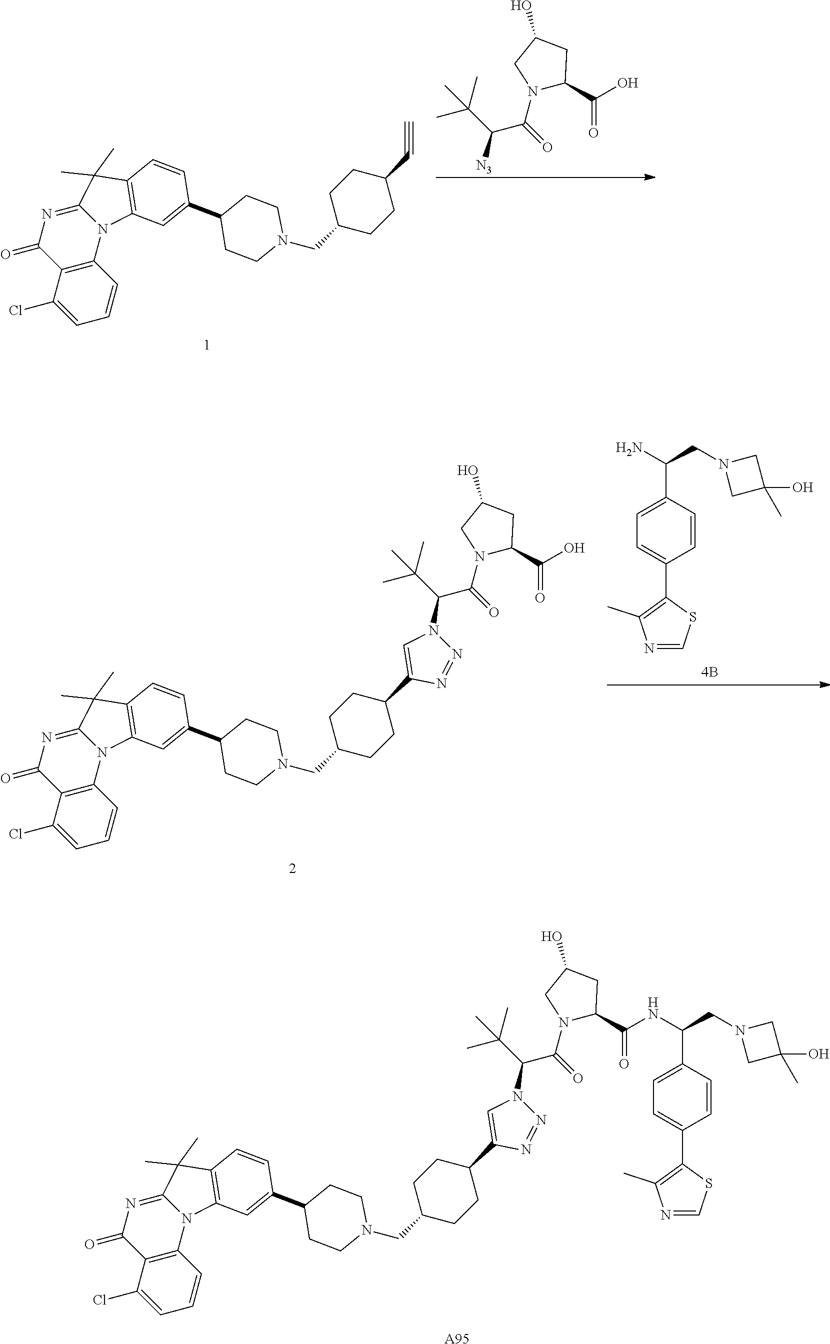

Compound A95 was synthesized using the similar procedures as used in the synthesis of Compound A67. ESI MS m/z: 1095.33 [M+H]⁺.

### Compound A96. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-1-(4-(4-methylthiazol-5-yl)phenyl)-2-(2-oxa-6-azaspiro[3.3]heptan-6-yl)ethyl)pyrrolidine-2-carboxamide

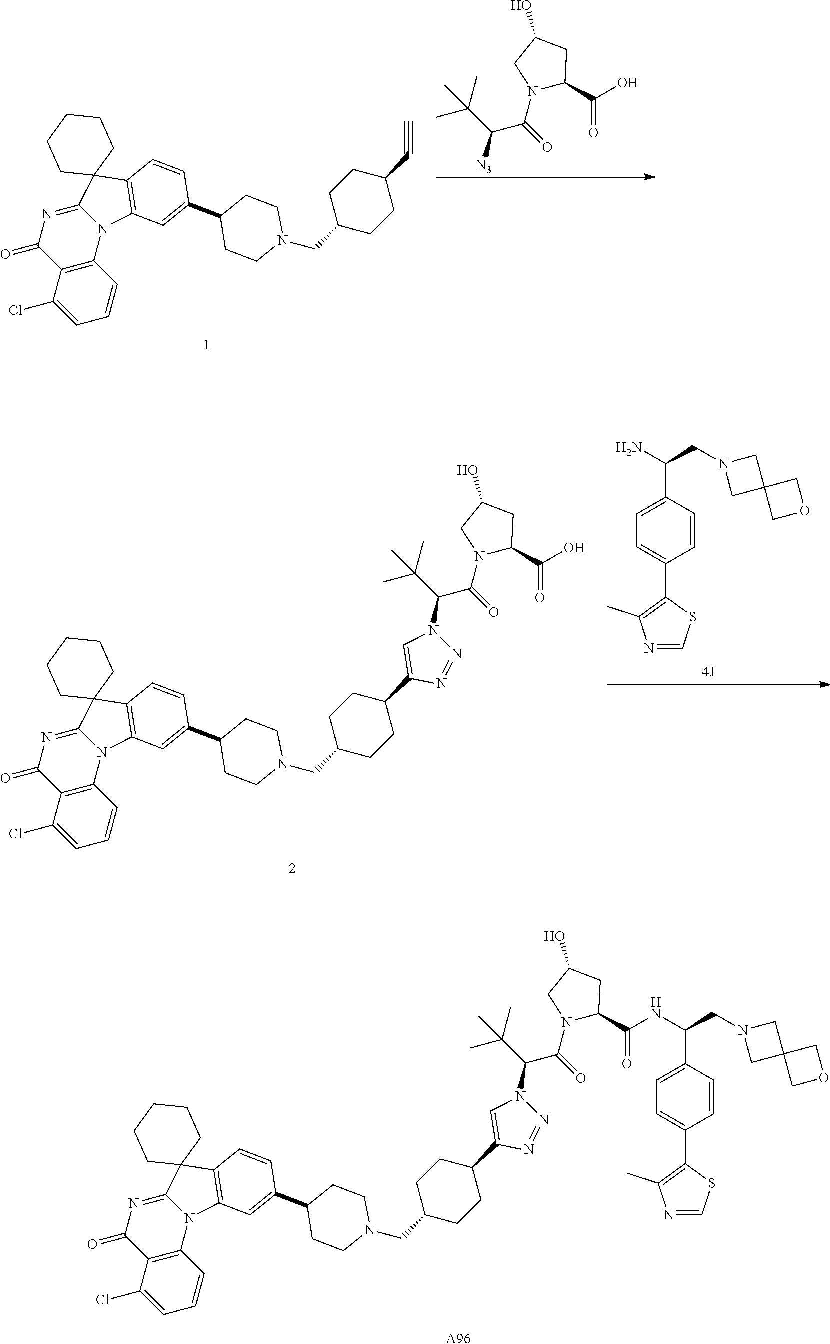

Compound A96 was synthesized using the similar procedures as used in the synthesis of Compound A67. ESI MS m/z: 1107.37 [M+H]⁺.

### Compound A97. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-1-(4-(4-methylthiazol-5-yl)phenyl)-2-(2-oxa-6-azaspiro[3.4]octan-6-yl)ethyl)pyrrolidine-2-carboxamide

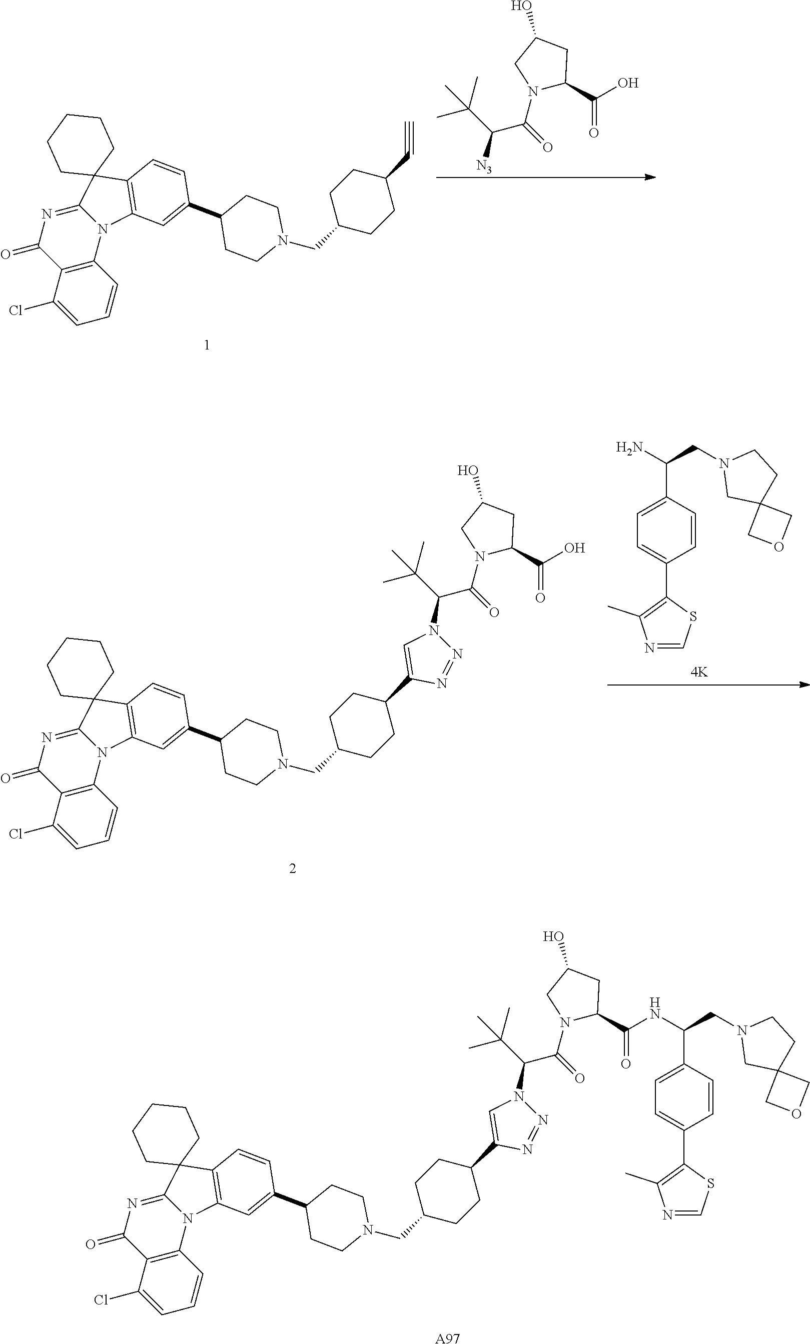

Compound A97 was synthesized using the similar procedures as used in the synthesis of Compound A67. ESI MS m/z: 1121.42 [M+H]⁺.

### Compound A98. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-hydroxy-N—((R)-1-(4-(4-methylthiazol-5-yl)phenyl)-2-(2-oxa-6-azaspiro[3.3]heptan-6-yl)ethyl)pyrrolidine-2-carboxamide

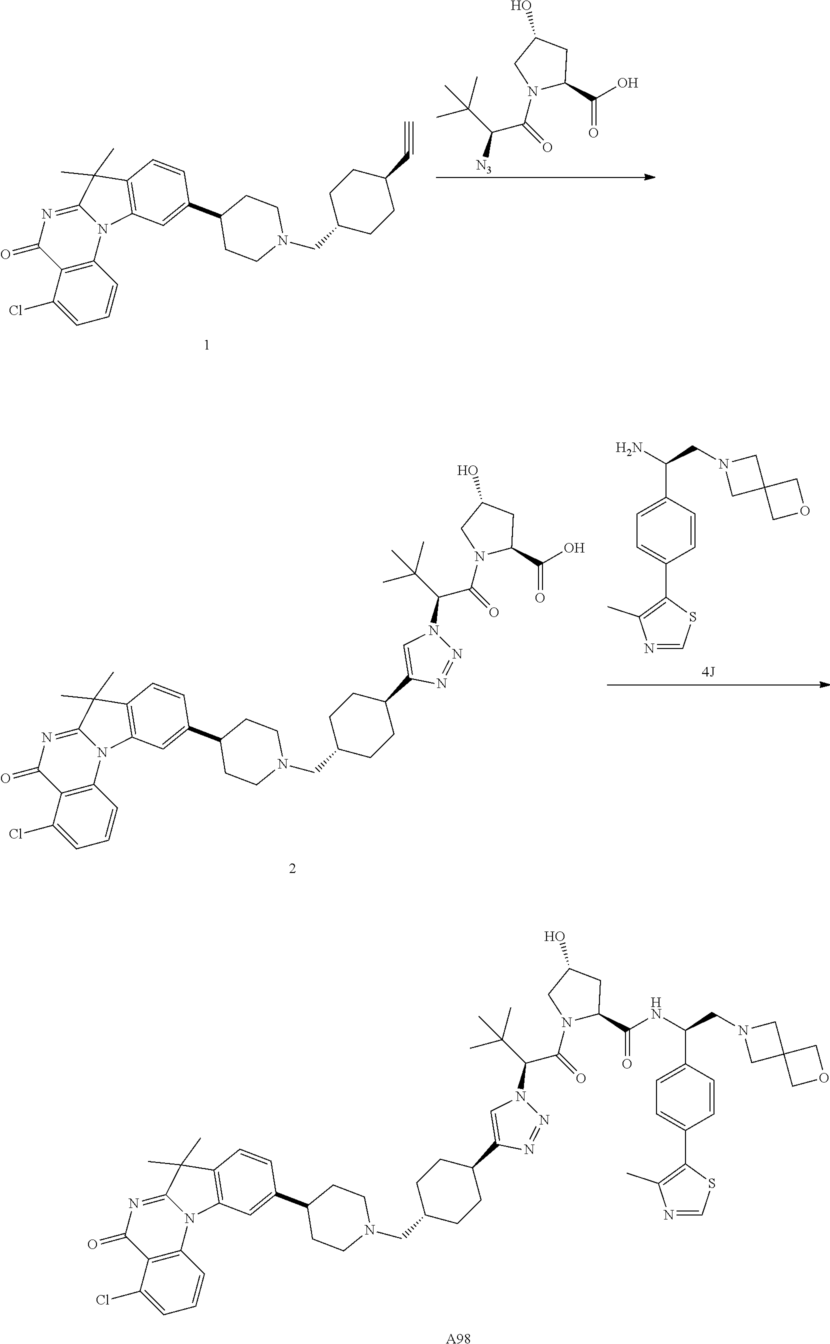

Compound A98 was synthesized using the similar procedures as used in the synthesis of Compound A67. ESI MS m/z: 1067.47 [M+H]⁺.

### Compound A99. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-hydroxy-N—((R)-1-(4-(4-methylthiazol-5-yl)phenyl)-2-(2-oxa-6-azaspiro[3.4]octan-6-yl)ethyl)pyrrolidine-2-carboxamide

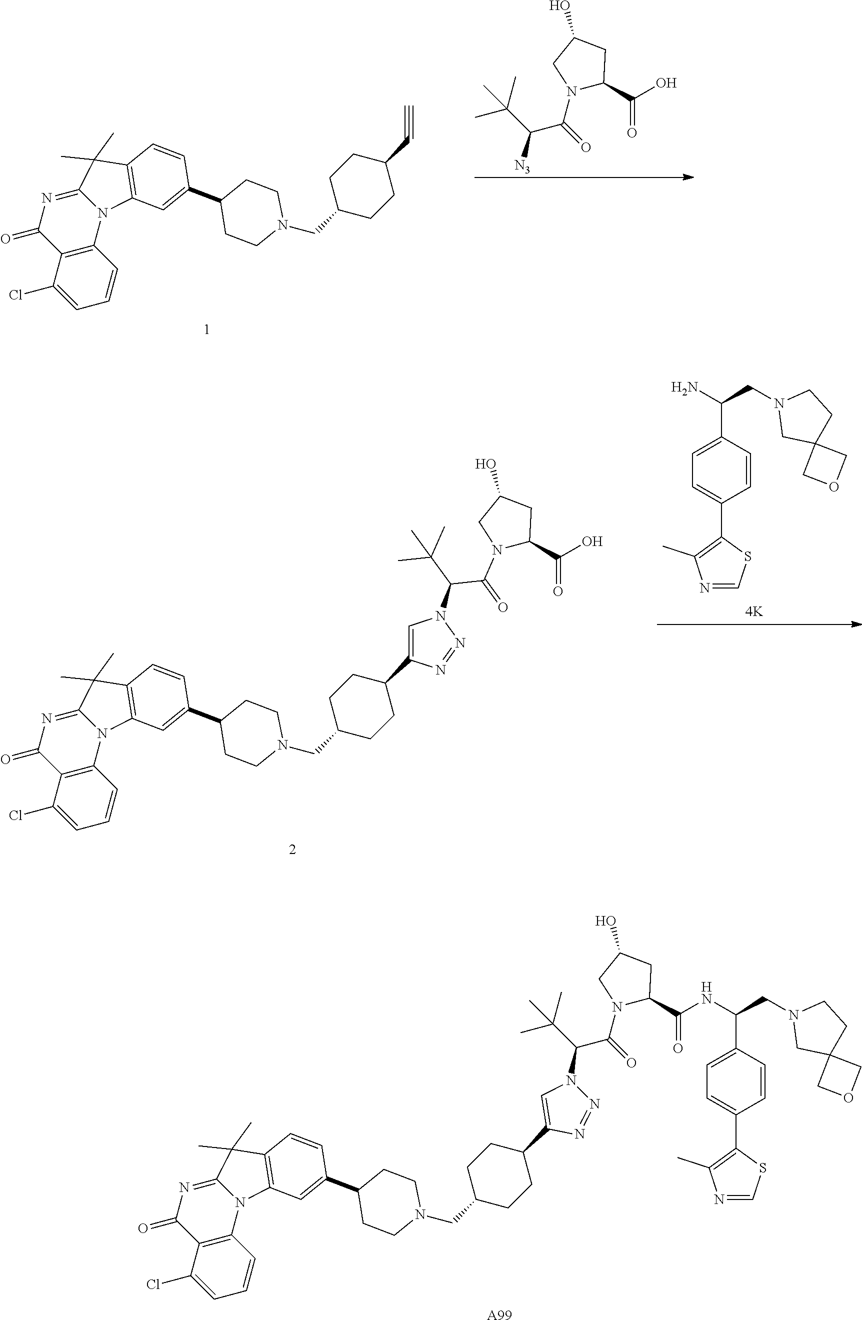

Compound A99 was synthesized using the similar procedures as used in the synthesis of Compound A67. ESI MS m/z: 1081.22 [M+H]⁺.

### Compound A100. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-(tetrahydro-2H-pyran-4-yl) acetyl)-4-hydroxy-N—((R)-1-(4-(4-methylthiazol-5-yl)phenyl)-2-morpholinoethyl)pyrrolidine-2-carboxamide

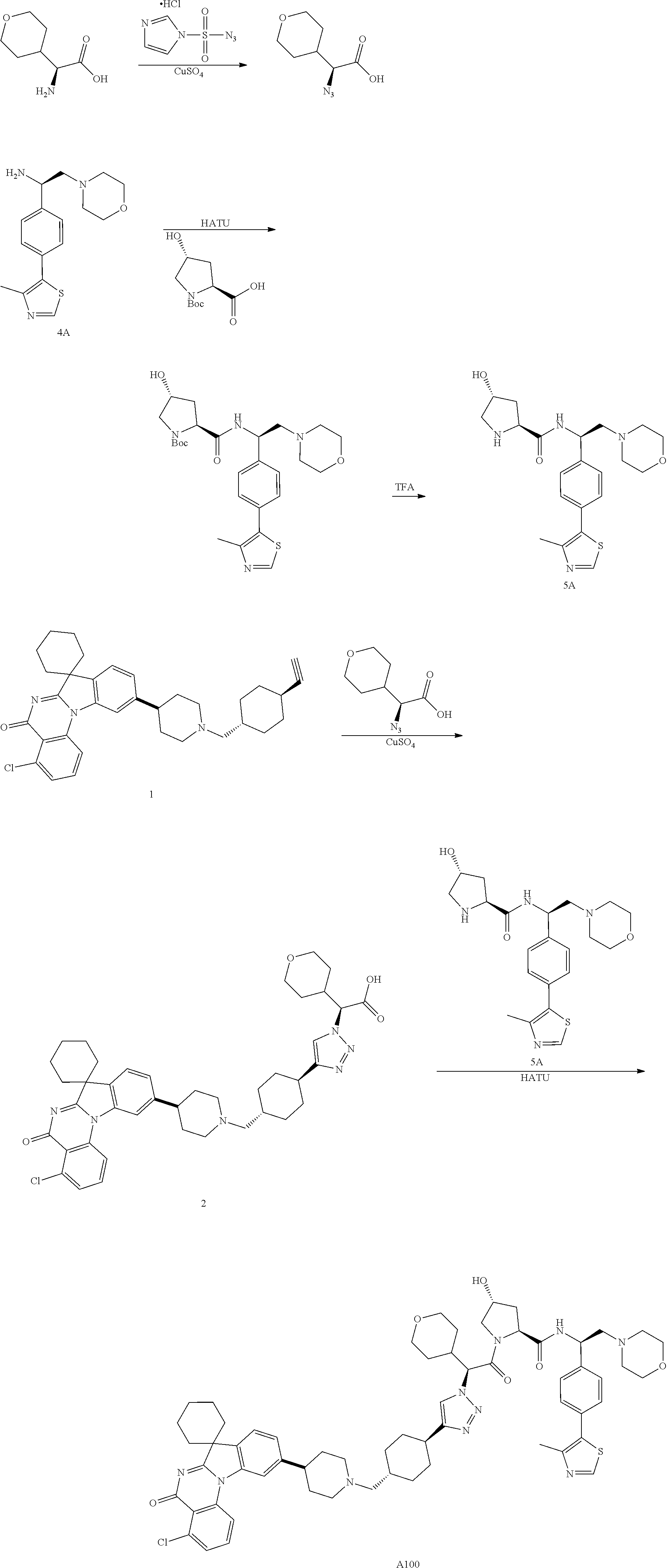

Preparation of 5A: To a flask was added(S)-2-amino-2-(tetrahydro-2H-pyran-4-yl) acetic acid (509 mg, 3.2 mmol, 1.0 eq), 1H-imidazole-1-sulfonyl azide hydrochloride (804 mg, 3.8 mmol, 1.2 eq), K₂CO₃ (1.1 g, 8 mmol, 2.5 eq), CuSO₄ (102 mg, 0.64 mmol, 0.2 eq). Then the solvent of MeOH (34 mL) was added to the flask. The suspension was stirred at rt for 18 h. The reaction mixture was diluted with water (40 mL) and concentrated in vacuum to remove methanol. The aqueous phase was adjust to pH=3 with potassium bisulfate and extracted with ethyl acetate (100 mL). The organic layer was separated and concentrated to give crude(S)-2-azido-2-(tetrahydro-2H-pyran-4-yl) acetic acid (462 mg, 78% yield).

To a solution of (2S,4R)-1-(tert-butoxycarbonyl)-4-hydroxypyrrolidine-2-carboxylic acid (288 mg, 1.25 mmol, 1.3 equiv.) and 4A (400 mg, 0.96 mmol, 1.0 equiv.) in DMF (8 mL) was added HATU (474 mg, 1.25 mmol, 1.3 equiv.) and DIPEA (894 μL, 3.76 mmol, 6 equiv.). The mixture was stirred for 10 min. The reaction is quenched by TFA, the residue was purified by pre-HPLC to get pure compound tert-butyl (2S,4R)-4-hydroxy-2-(((R)-1-(4-(4-methylthiazol-5-yl)phenyl)-2-morpholinoethyl) carbamoyl)pyrrolidine-1-carboxylate (441 mg, 89%).

To a solution of tert-butyl (2S,4R)-4-hydroxy-2-(((R)-1-(4-(4-methylthiazol-5-yl)phenyl)-2-morpholinoethyl) carbamoyl)pyrrolidine-1-carboxylate (441 mg, 1 equiv.) in DCM (10 mL) was added TFA (3 mL) and the mixture was stirred for 60 min. After removing the solvent, the product 5A was received and dried by lyophilizer, and then the crude product can be used in the next step directly.

To a solution of 1 (196 mg, 0.3 mmol) in water (1 mL) and tert-butyl alcohol (1 mL) was added crude(S)-2-azido-2-(tetrahydro-2H-pyran-4-yl) acetic acid (112 mg, 0.60 mmol), copper sulfate (39 mg, 0.16 mmol) and sodium Lascorbate (130 mg, 0.66 mmol). The reaction was stirred at 25° C. for 5 h, the mixture was subjected to reverse phase flash chromatography (10-100% MeCN in H₂O, 0.1% TFA) to give compound 2 (192 mg, 85%).

To a solution of 2 (81 mg, 0.096 mmol, 1.0 equiv.) and 5A (61.5 mg, 0.115 mmol, 1.2 equiv.) in DMF (2 mL) was added HATU (40 mg, 0.106 mmol, 1.1 equiv.) and DIPEA (120 μL, 0.77 mmol, 8 equiv.). The mixture was stirred for 10 min. The reaction is quenched by TFA, the residue was purified by pre-HPLC to get pure compound A100 (97 mg, 90%). ESI MS m/z: 1123.51 [M+H]⁺.

Compound A101 was synthesized using the similar procedures as used in the synthesis of Compound A100. ESI MS m/z: 1167.63 [M+H]⁺.

Compound A102 was synthesized using the similar procedures as used in the synthesis of Compound A100. ESI MS m/z: 1083.50 [M+H]⁺.

Compound A103 was synthesized using the similar procedures as used in the synthesis of Compound A100. ESI MS m/z: 1081.56 [M+H]⁺.

Compound A104 was synthesized using the similar procedures as used in the synthesis of Compound A100. ESI MS m/z: 1041.76 [M+H]⁺.

### Compound A105. (2S,4R)-1-((S)-2-(1-(2-(4-(4′-chloro-5′-oxo-5′H-spiro[cyclohexane-1,7′-indolo[1,2-a]quinazolin]-10′-yl)piperidin-1-yl) acetyl)piperidin-4-yl)-2-(4-cyclopropyl-1H-1,2,3-triazol-1-yl) acetyl)-4-hydroxy-N—((S)-1-(4-(4-methylthiazol-5-yl)phenyl)ethyl)pyrrolidine-2-carboxamide

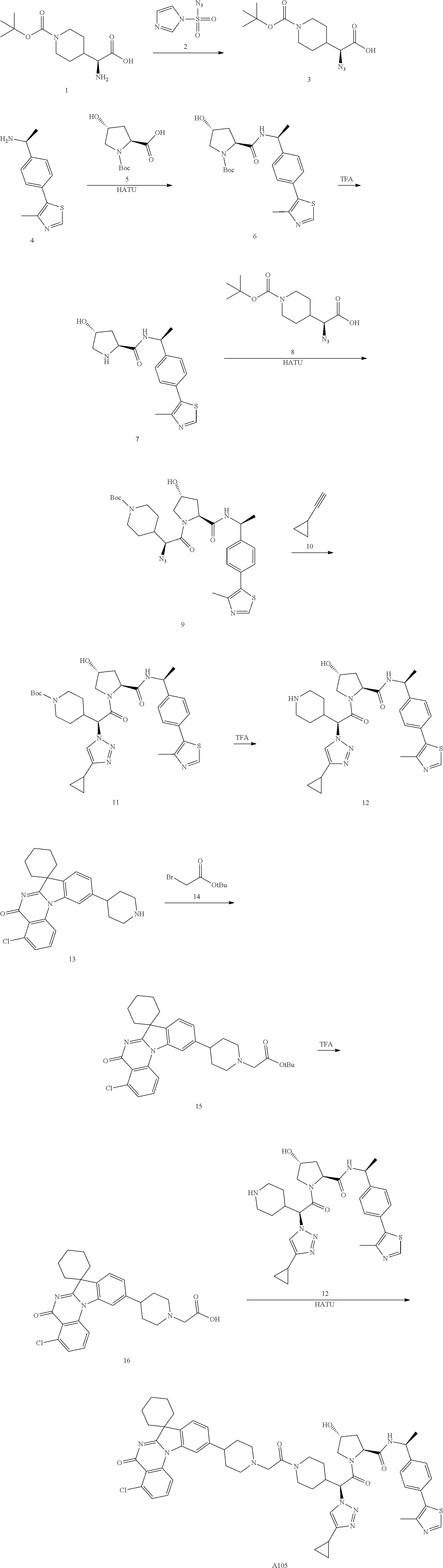

### Synthesis of(S)-2-azido-2-(1-(tert-butoxycarbonyl)piperidin-4-yl) acetic acid, 3

To suspension of 1 (1.00 g, 3.87 mmol, 1 eq) in MeOH (50 mL) was added 2 (1.00 g, 5.81 mmol, 1.5 eq), K₂CO₃ (1.07 g, 7.74 mmol, 2.0 eq), CuSO₄ (1 g, 3.87 mmol, 1 eq). Then the suspension was stirred for 16 h. After reaction, the mixture was concentrated. The residue was dissolved into a mixture of ethyl acetate and water, HCl was added to turn the pH to 2-3. The ethyl acetate phase was collected and the aqueous phase was extracted by ethyl acetate once. Combined organic phases were washed by brine once. After dried by anhydrous Na₂SO₄ and concentrated, the crude product 3 (1.2 g) was obtained and used in the next step directly.

### Synthesis of (2S,4R)-4-hydroxy-N—((S)-1-(4-(4-methylthiazol-5-yl)phenyl)ethyl)pyrrolidine-2-carboxamide, 7

To a solution of 4 (2.00 g, 9.16 mmol, 1.0 eq), 5 (2.12 g, 9.16 mmol, 1.0 eq), HATU (5.22 g, 13.74 mmol, 1.5 eq) in DMF (50 mL) was added DIPEA (3.55 g, 27.48 mmol, 3.0 eq). The mixture was stirred for 30 minutes. The reaction was quenched by adding water. The mixture was extracted by ethyl acetate (80*2 mL) twice. Combined organic phases were washed by water twice. After dried by anhydrous Na₂SO₄ and concentrated. The received residue was dissolved into DCM (40 mL), TFA (20 mL) was added, the mixture stirred for 30 minutes. After reaction, the solution was concentrated. The residue was purified by C-18 reversal column chromatography (elution solvents: CH₃CN/H₂O from 10% to 100%, 0.1% TFA) to get target product 7 (1.6 g, 53%) as a slight yellow solid. ESI MS m/z: 332.13 [M+H]⁺.

### Synthesis of tert-butyl 4-((S)-1-azido-2-((2S,4R)-4-hydroxy-2-(((S)-1-(4-(4-methylthiazol-5-yl)phenyl)ethyl) carbamoyl) pyrrolidin-1-yl)-2-oxoethyl)piperidine-1-carboxylate, 9

To a solution of 7 (1.00 g, 3.02 mmol, 1.0 eq), 8 (857 mg, 3.02 mmol, 1.0 eq), HATU (1.72 g, 4.52 mmol, 1.5 eq) in DMF (50 mL) was added DIPEA (1.17 g, 9.05 mmol, 3.0 eq). The mixture was stirred for 30 minutes. The reaction was quenched by adding water. The mixture was extracted by ethyl acetate (60*2 mL) twice. Combined organic phases were washed by water twice. After dried by anhydrous Na₂SO₄ and concentrated. The residue was purified by C-18 reversal column chromatography (elution solvents: CH₃CN/H₂O from 10% to 100%, 0.1% TFA) to get target product 9 (1.3 g, 72%) as a slight yellow solid. ESI MS m/z: 598.28 [M+H]⁺.

### Synthesis of (2S,4R)-1-((S)-2-(4-cyclopropyl-1H-1,2,3-triazol-1-yl)-2-(piperidin-4-yl) acetyl)-4-hydroxy-N—((S)-1-(4-(4-methylthiazol-5-yl)phenyl)ethyl)pyrrolidine-2-carboxamide, 12

A solution of 9 (1.00 g, 1.67 mmol, 1.0 eq), 10 (332 mg, 5.02 mmol, 3.0 eq), sodium ascorbate (666 mg, 3.35 mmol, 2.0 eq), CuSO₄·10 (H₂O) (417 mg, 1.67 mmol, 1.0 eq) in t-BuOH/H₂O (50/25 mL) was stirred overnight. The reaction solution was concentrated. The mixture was redissolved into a mixture of EA/H₂O (80/80 mL), the organic phase was collected and washed by water once. After dried by anhydrous Na₂SO₄ and concentrated. The residue was received and redissolved into DCM (40 mL). TFA (20 mL) was added, the mixture stirred for 30 minutes. After reaction the solution was concentrated. The residue was purified by C-18 reversal column chromatography (elution solvents: CH₃CN/H₂O from 10% to 100%, 0.1% TFA) to get target product 12 (650 mg, 69%) as a slight yellow solid. ESI MS m/z: 564.28 [M+H]⁺.

### Synthesis of 2-(4-(4′-chloro-5′-oxo-5′H-spiro[cyclohexane-1,7′-indolo[1,2-a]quinazolin]-10′-yl)piperidin-1-yl) acetic acid, 16

To a solution of 13 (100 mg, 0.24 mmol, 1.0 eq), 14 (90 mg, 0.48 mmol, 2.0 eq) in DMF (5 mL) was added DIPEA (123 mg, 0.95 mmol, 4.0 eq). The mixture was stirred overnight at 80° C. The reaction was quenched by added water. The mixture was extracted by ethyl acetate (40*2 mL) twice. Combined organic phases were washed by water twice. After dried by anhydrous Na₂SO₄ and concentrated. The received residue was dissolved into DCM (10 mL), TFA (10 mL) was added, the mixture stirred for 3 h. After reaction the solution was concentrated. The residue was purified by C-18 reversal column chromatography (elution solvents: CH₃CN/H₂O from 10% to 100%, 0.1% TFA) to get target product 16 (76 mg, 67%) as a solid. ESI MS m/z: 478.18 [M+H]⁺.

### Synthesis of (2S,4R)-1-((S)-2-(1-(2-(4-(4′-chloro-5′-oxo-5′H-spiro[cyclohexane-1,7′-indolo[1,2-a]quinazolin]-10′-yl)piperidin-1-yl) acetyl)piperidin-4-yl)-2-(4-cyclopropyl-1H-1,2,3-triazol-1-yl) acetyl)-4-hydroxy-N—((S)-1-(4-(4-methylthiazol-5-yl)phenyl)ethyl)pyrrolidine-2-carboxamide, A130

To a solution of 16 (30 mg, 0.62 mmol, 1.0 eq), 12 (35 mg, 0.62 mmol, 1.0 eq), HATU (35 mg, 0.94 mmol, 1.5 eq), in DMF (5 mL) was added DIPEA (24 mg, 0.18 mmol, 3.0 eq). The mixture was stirred for 30 minutes. The mixture solution was purified directly by pre-HPLC to get product A¹⁰⁵ (32 mg, 50%). ESI MS m/z: 1023.44 [M+H]⁺.

### Compound A106. (2S,4R)-1-((S)-2-(1-(4-(4-(4′-chloro-5′-oxo-5′H-spiro[cyclohexane-1,7′-indolo[1,2-a]quinazolin]-10′-yl)piperidin-1-yl)butanoyl)piperidin-4-yl)-2-(4-cyclopropyl-1H-1,2,3-triazol-1-yl) acetyl)-4-hydroxy-N—((S)-1-(4-(4-methylthiazol-5-yl)phenyl)ethyl)pyrrolidine-2-carboxamide

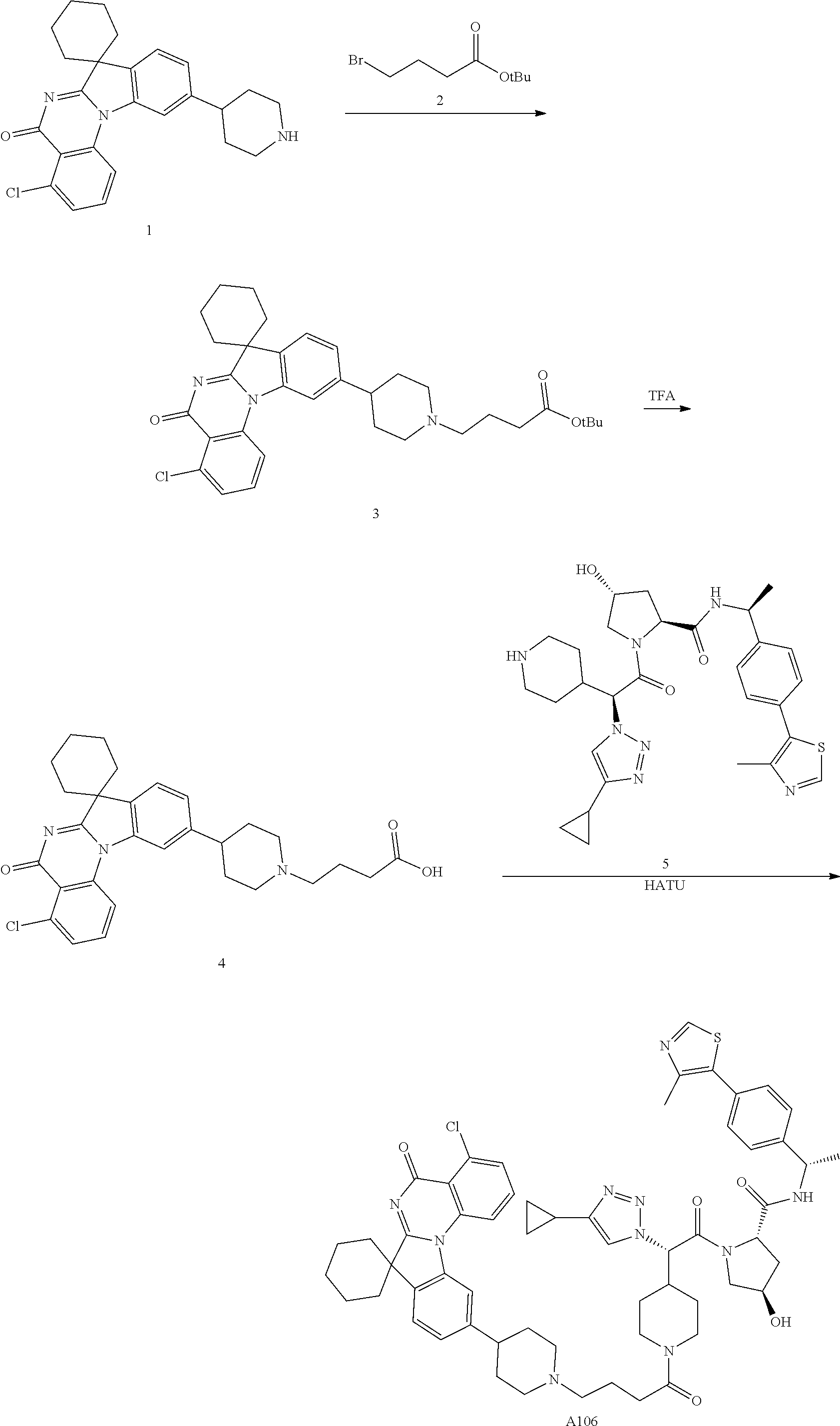

The reaction routine showed above figure. The procedure is similar as the preparation of A¹⁰⁵ except changing the recording reagents. The target degrader was obtained as while solid. ESI MS m/z: 1051.47 [M+H]⁺.

### Compound A107. (2S,4R)-1-((S)-2-(1-(5-(4-(4′-chloro-5′-oxo-5′H-spiro[cyclohexane-1,7′-indolo[1,2-a]quinazolin]-10′-yl)piperidin-1-yl) pentanoyl)piperidin-4-yl)-2-(4-cyclopropyl-1H-1,2,3-triazol-1-yl) acetyl)-4-hydroxy-N—((S)-1-(4-(4-methylthiazol-5-yl)phenyl)ethyl)pyrrolidine-2-carboxamide

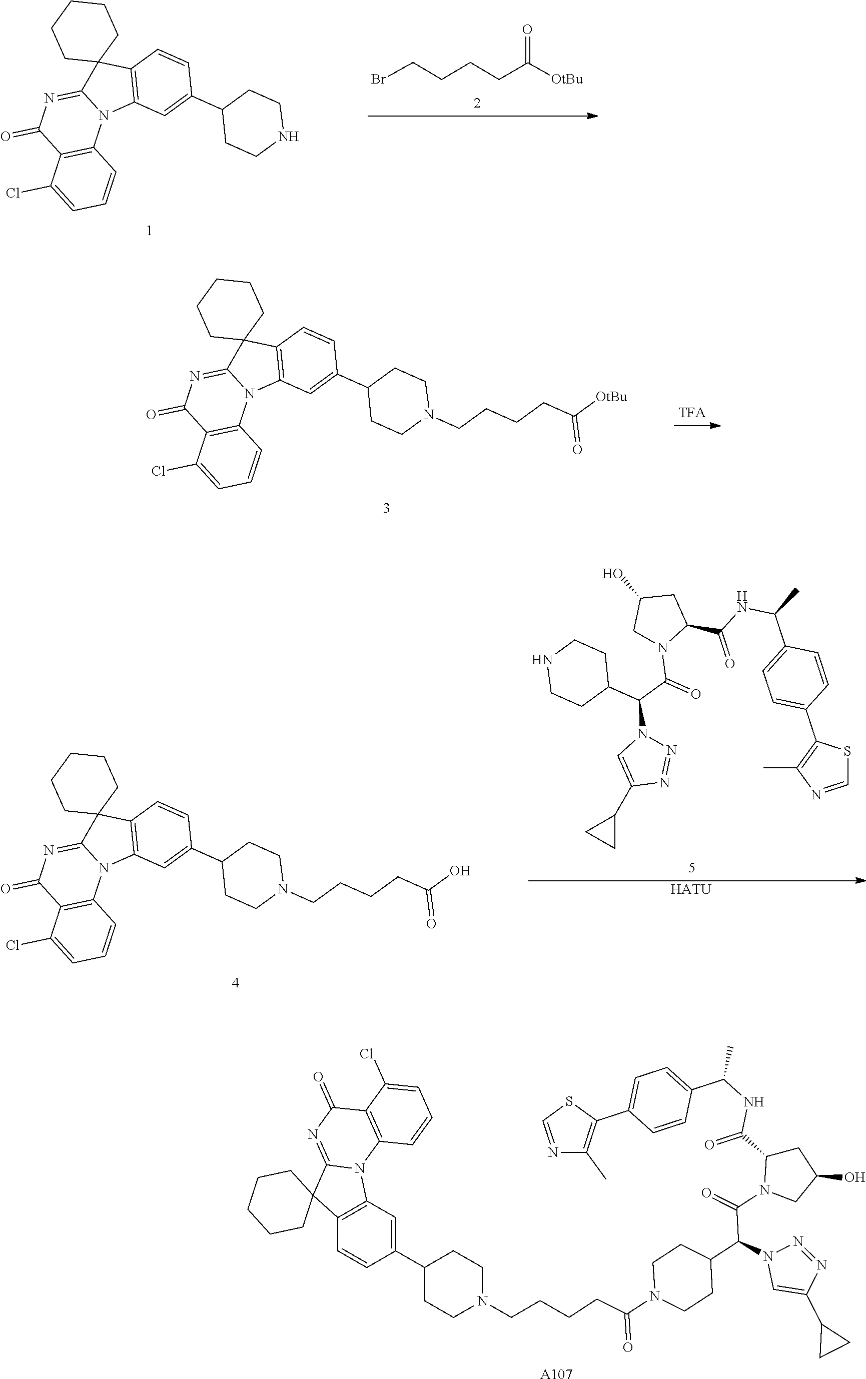

The reaction routine showed above figure. The procedure is similar as the preparation of A¹⁰⁵ except changing the recording reagents. The target degrader was obtained as while solid. ESI MS m/z: 1065.48 [M+H]⁺.

### Compound A108. (2S,4R)-1-((S)-2-(1-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexane-1-carbonyl)piperidin-4-yl)-2-(4-cyclopropyl-1H-1,2,3-triazol-1-yl) acetyl)-4-hydroxy-N—((S)-1-(4-(4-methylthiazol-5-yl)phenyl)ethyl)pyrrolidine-2-carboxamide

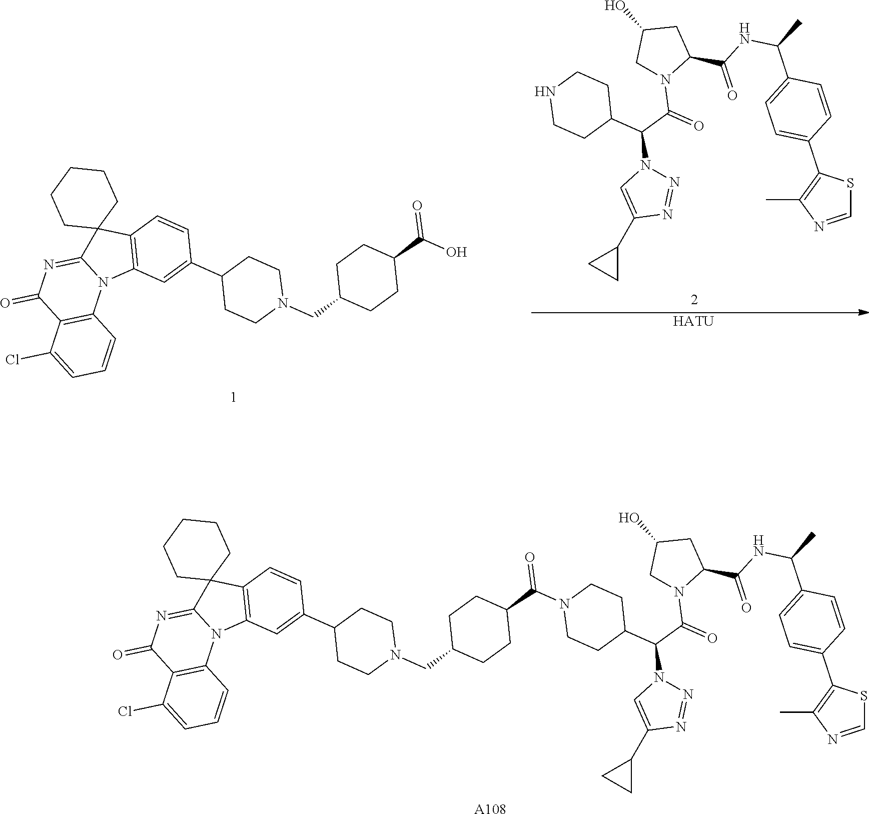

The reaction routine showed above figure. The procedure is similar as the preparation of A¹⁰⁵ except changing the recording reagents. The target degrader was obtained as while solid. ESI MS m/z: 1065.48 [M+H]⁺.

### Compound A109. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-(piperidin-4-yl) acetyl)-4-hydroxy-N—((S)-1-(4-(4-methylthiazol-5-yl)phenyl)ethyl)pyrrolidine-2-carboxamide

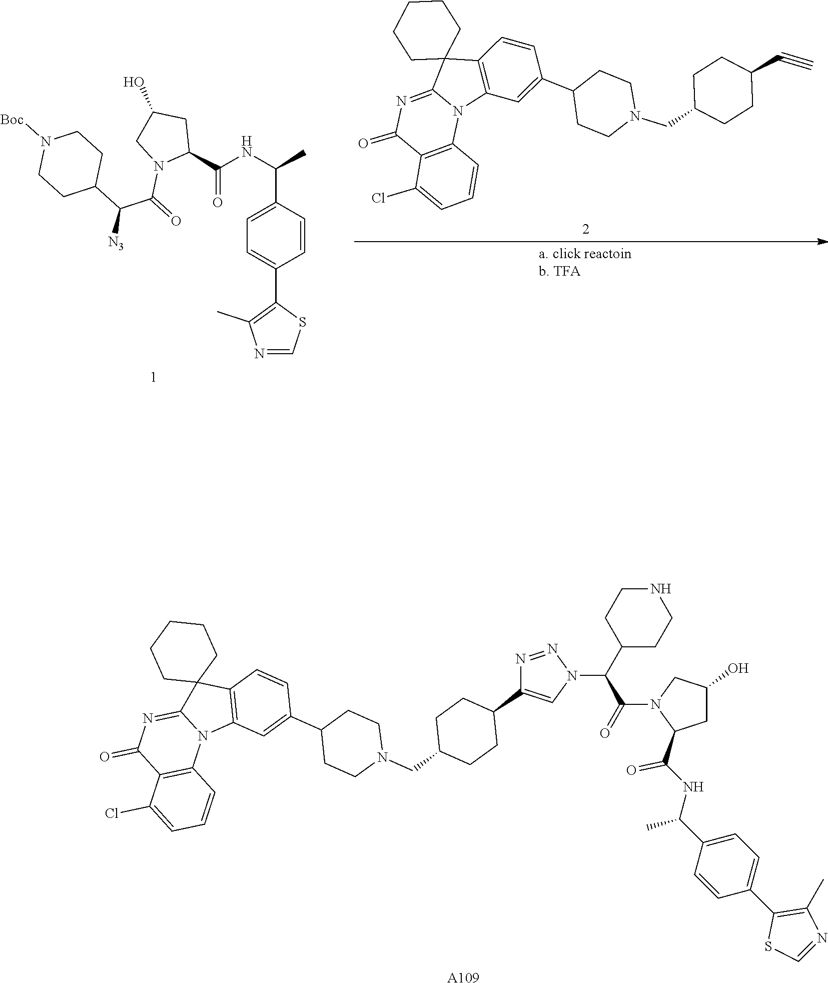

A solution of 1 (30 mg, 0.05 mmol, 1.0 eq), 2 (27 mg, 0.05 mmol, 1.0 eq), sodium ascorbate (20 mg, 0.1 mmol, 2.0 eq), CuSO₄·10 (H₂O) (12.5 mg, 0.05 mmol, 1.0 eq) in t-BuOH/H₂O (20/10 mL) was stirred overnight. The reaction solution was concentrated. The mixture was redissolved into a mixture of EA/H₂O (50/50 mL), the organic phase was collected and washed by water once. After dried by anhydrous Na₂SO₄ and concentrated. The residue was received and redissolved into DCM (10 mL). TFA (5 mL) was added, the mixture stirred for 30 minutes. After reaction the solution was concentrated. The residue was purified by pre-HPLC (elution solvents: CH₃CN/H₂O from 10% to 100%, 0.1% TFA) to get target product 3 (30 mg, 56%) as a slight yellow solid. ESI MS m/z: 1037.50 [M+H]⁺.

### Compound A110. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-(1-methylpiperidin-4-yl) acetyl)-4-hydroxy-N—((S)-1-(4-(4-methylthiazol-5-yl)phenyl)ethyl)pyrrolidine-2-carboxamide

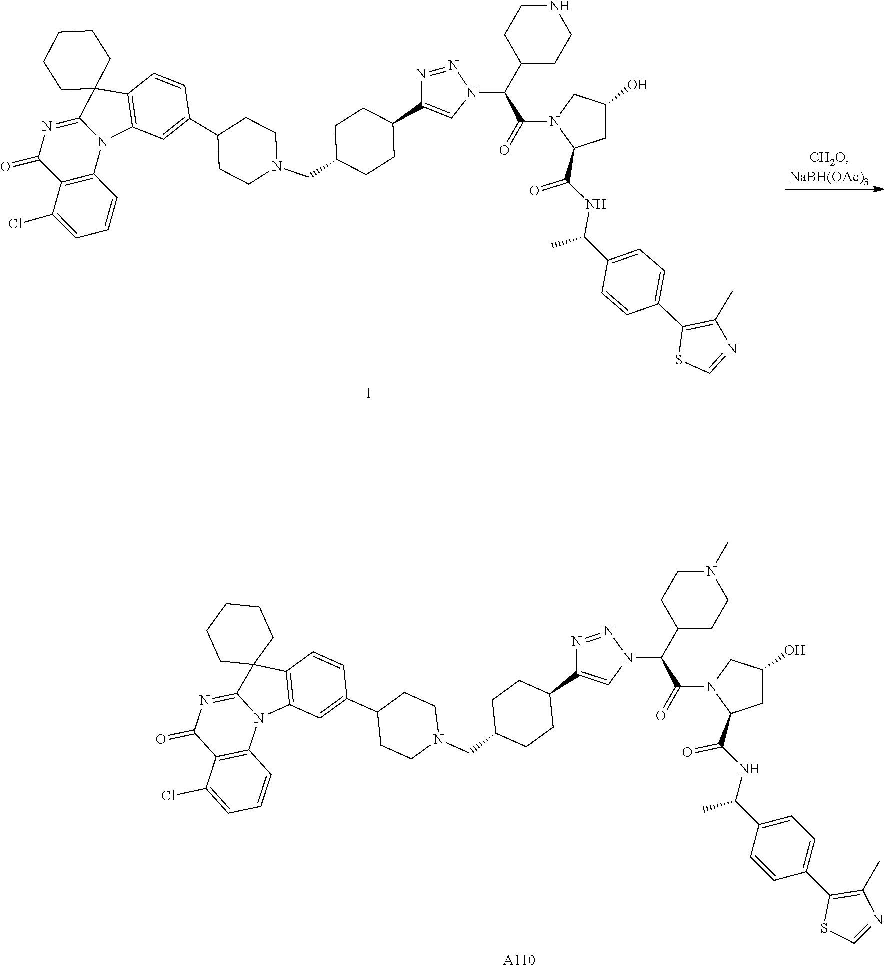

A solution of 1 (30 mg, 0.028 mmol, 1.0 eq), formic aldehyde (5.2 mg, 0.086 mmol, 3.0 eq), and NaBH(OAc)₃ (30.6 mg, 0.14 mmol, 5.0 eq) in DMF/DCE (2/2 mL) was stirred 3 h. The mixture solution was purified directly by pre-HPLC to get product 17 (16 mg, 53%). ESI MS m/z: 1051.50 [M+H]⁺.

### Compound A111. (2S,4R)-1-((S)-2-(1-acetylpiperidin-4-yl)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl) acetyl)-4-hydroxy-N—((S)-1-(4-(4-methylthiazol-5-yl)phenyl)ethyl)pyrrolidine-2-carboxamide

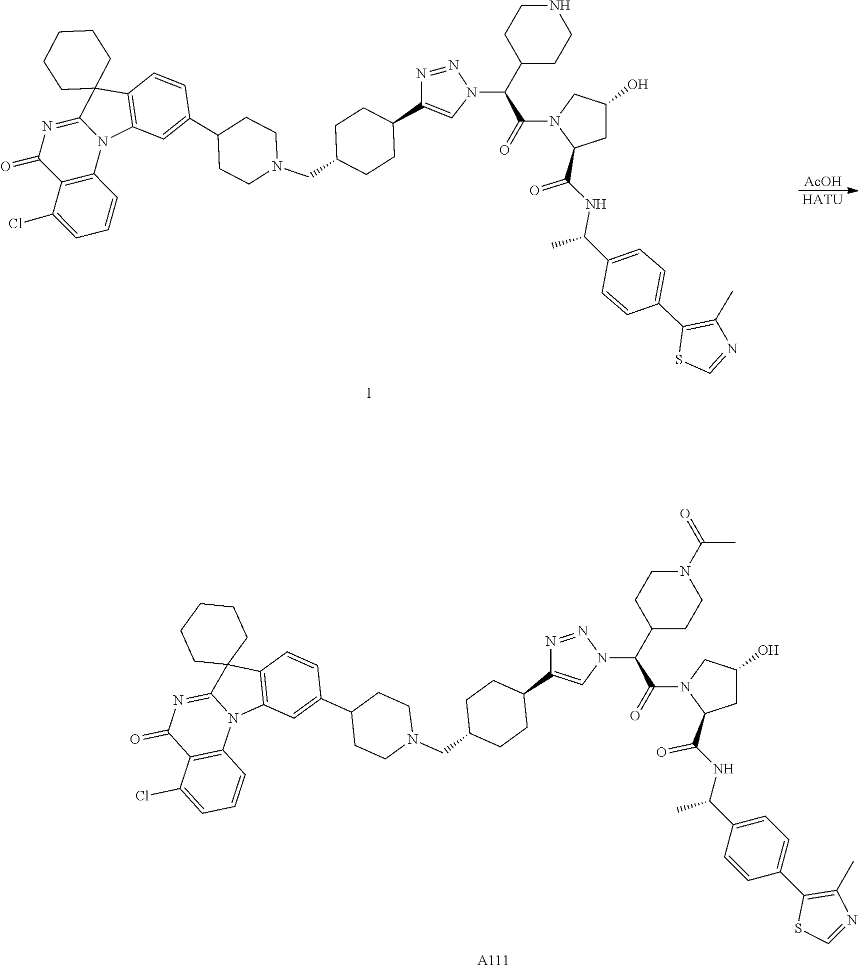

To a solution of 1 (30 mg, 0.029 mmol, 1.0 eq), AcOH (5.2 mg, 0.086 mmol, 3.0 eq), HATU (16.5 mg, 0.043 mmol, 1.5 eq), in DMF (3 mL) was added DIPEA (18.6 mg, 0.14 mmol, 5.0 eq). The mixture was stirred for 30 minutes. The mixture solution was purified directly by pre-HPLC to get product 2 (23 mg, 74%). ESI MS m/z: 1079.50 [M+H]⁺.

### Compound A112. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-(1-(tetrahydro-2H-pyran-4-yl)piperidin-4-yl) acetyl)-4-hydroxy-N—((S)-1-(4-(4-methylthiazol-5-yl)phenyl)ethyl)pyrrolidine-2-carboxamide

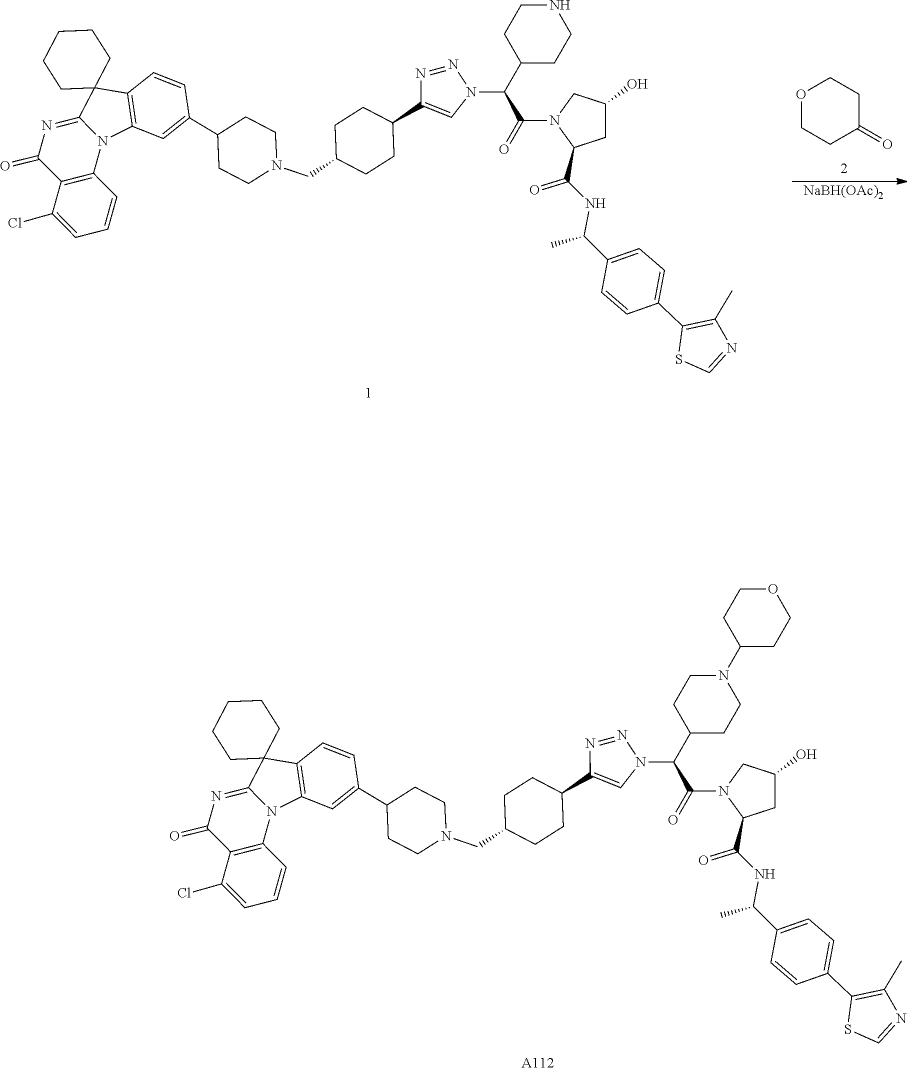 [SMILES: Cc1ncsc1-c1ccc([C@H](C)NC(=O)[C@@H]2C[C@@H](O)CN2C(=O)[C@H](C2CCNCC2)n2cc([C@H]3CC[C@H](CN4CCC(c5ccc6c(c5)-n5c(nc(=O)c7c(Cl)cccc75)C65CCCCC5)CC4)CC3)nn2)cc1 ; O=C1CCOCC1 ; Cc1ncsc1-c1ccc([C@H](C)NC(=O)[C@@H]2C[C@@H](O)CN2C(=O)[C@H](C2CCN(C3CCOCC3)CC2)n2cc([C@H]3CC[C@H](CN4CCC(c5ccc6c(c5)-n5c(nc(=O)c7c(Cl)cccc75)C65CCCCC5)CC4)CC3)nn2)cc1 ; "NaBH(OAc)2" ; "A112"]

A solution of 1 (30 mg, 0.029 mmol, 1.0 eq), (14.5 mg, 0.086 mmol, 5.0 eq), and NaBH(OAc)₃ (61.3 mg, 0.29 mmol, 10.0 eq) in DMF/DCE (2/2 mL) was stirred 16 h. The mixture solution was purified directly by pre-HPLC to get product (12 mg, 37%). ESI MS m/z: 1021.55 [M+H]⁺.

### Compound A113. (2S,4R)-1-((S)-2-(1-((S)-1,4-dioxane-2-carbonyl)piperidin-4-yl)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl) acetyl)-4-hydroxy-N—((S)-1-(4-(4-methylthiazol-5-yl)phenyl)ethyl)pyrrolidine-2-carboxamide

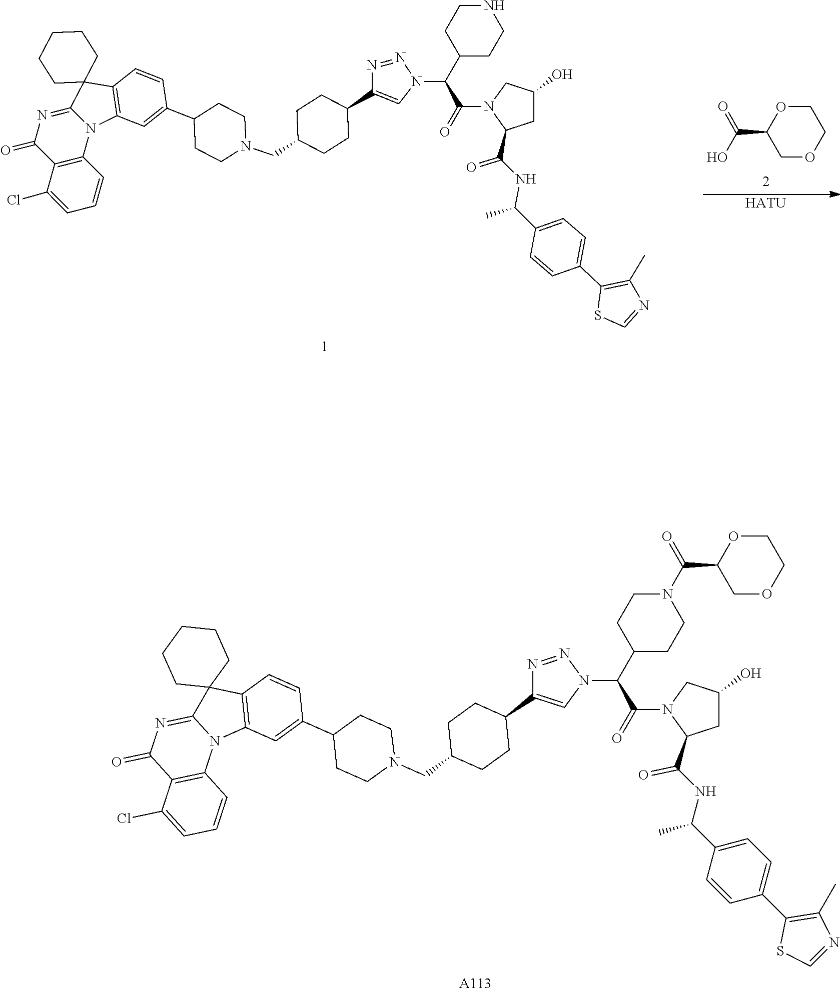

To a solution of 1 (30 mg, 0.029 mmol, 1.0 eq), 2 (5.8 mg, 0.043 mmol, 1.5 eq), HATU (16.5 mg, 0.043 mmol, 1.5 eq), in DMF (3 mL) was added DIPEA (11.6 mg, 0.086 mmol, 3.0 eq). The mixture was stirred for 30 minutes. The mixture solution was purified directly by pre-HPLC to get product 2 (21 mg, 63%). ESI MS m/z: 1150.52 [M+H]⁺.

### Compound A114. (2S,4R)—N—((R)-2-(4-((4-(4′-bromo-5′-oxo-5′H-spiro[cyclohexane-1,7′-indolo[1,2-a]quinazolin]-10′-yl)piperidin-1-yl)methyl)-1H-1,2,3-triazol-1-yl)-1-(4-(4-methylthiazol-5-yl)phenyl)ethyl)-1-((S)-2-(4-cyclopropyl-1H-1,2,3-triazol-1-yl)-3,3-dimethylbutanoyl)-4-hydroxypyrrolidine-2-carboxamide

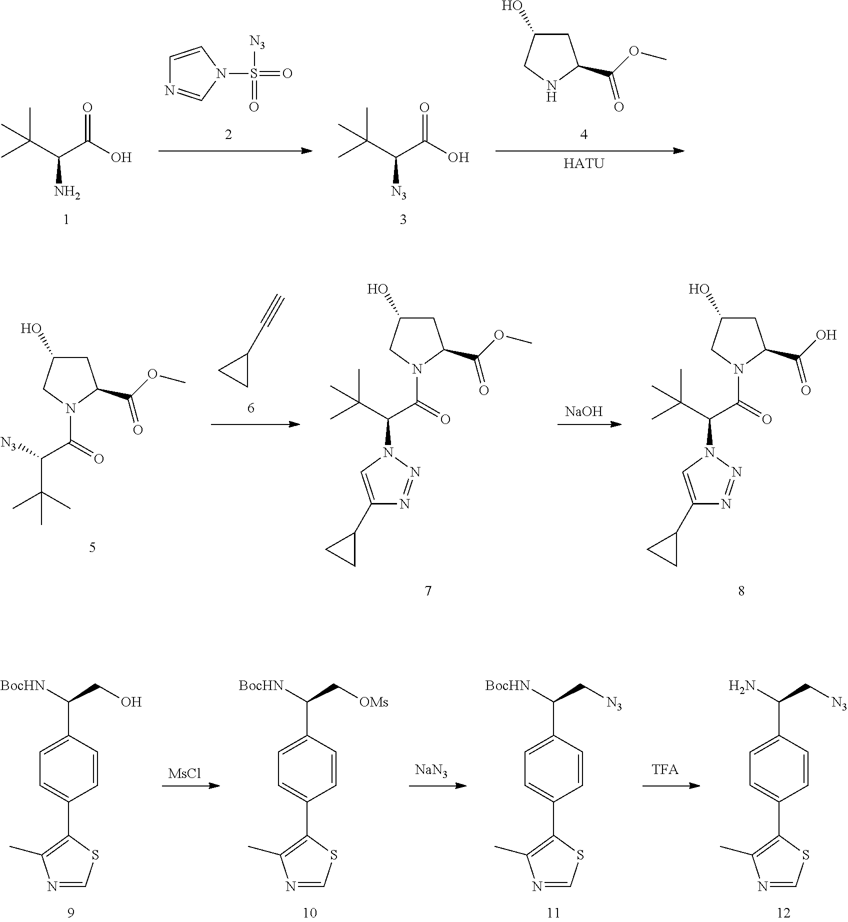

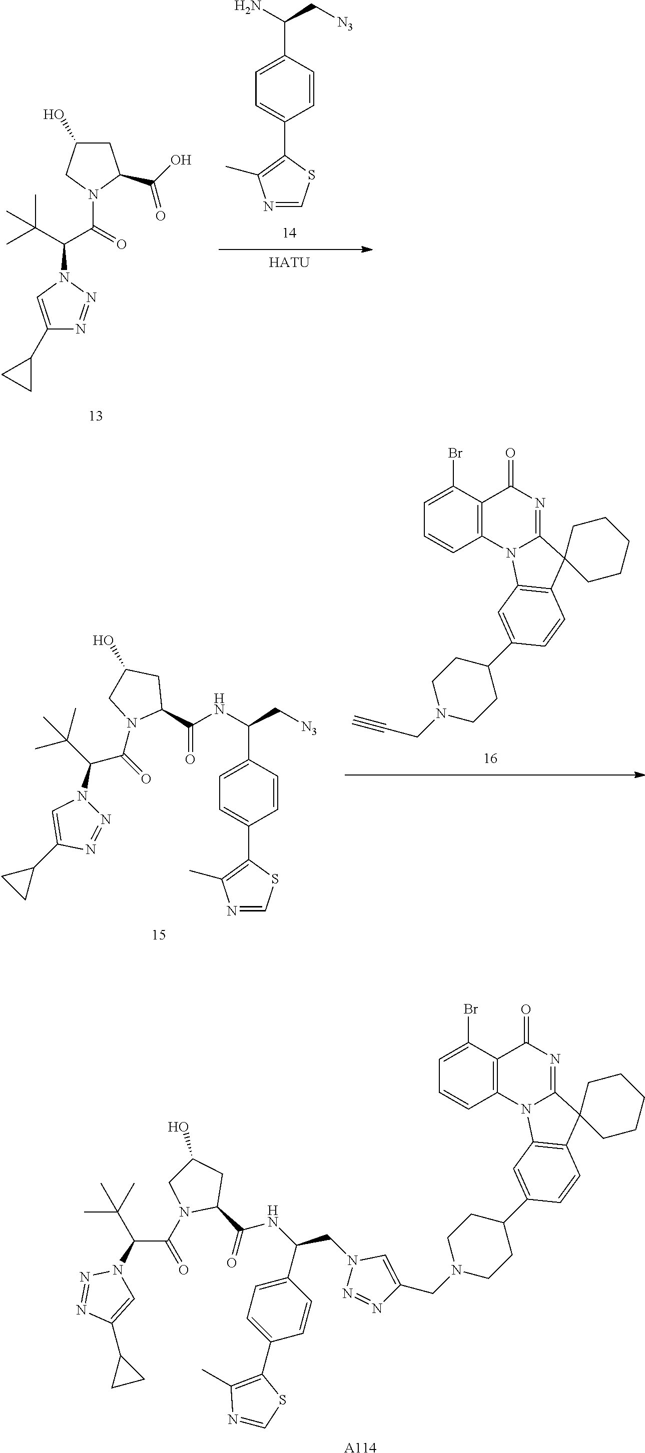

### Synthesis of methyl (2S,4R)-1-((S)-2-azido-3,3-dimethylbutanoyl)-4-hydroxypyrrolidine-2-carboxylate, 5

To suspension of 1 (500 mg, 3.87 mmol, 1 eq) in MeOH (50 mL) was added 2 (1.00 g, 5.81 mmol, 1.5 eq), K₂CO₃ (1.07 g, 7.74 mmol, 2.0 eq), CuSO₄ (121 mg, 0.76 mmol, 1.0 eq). Then the suspension was stirred for 16 h. After reaction, the mixture was concentrated. The residue was dissolved into a mixture of ethyl acetate and water, HCl was added to turn the pH to 2-3. The ethyl acetate phase was collected and the aqueous phase was extracted by ethyl acetate once. Combined organic phases were washed by brine once. After dried and concentrated, the crude product (0.6 g) was obtained and dissolved into DMF (10 mL) directly. Then 4 (554 g, 3.82 mmol, 1.0 eq), HATU (2.18 g, 13.74 mmol, 1.5 eq), DIPEA (1.48 g, 11.45 mmol, 3.0 eq). The mixture was stirred for 30 minutes. The reaction was quenched by added water. The mixture was extracted by ethyl acetate (80*2 mL) twice. Combined organic phases were washed by water twice. After dried by anhydrous Na₂SO₄ and concentrated. The residue was purified by C-18 reversal column chromatography (elution solvents: CH₃CN/H₂O from 10% to 100%, 0.1% TFA) to get target product 7 (0.7 g, 64%) as a slight yellow oil. ESI MS m/z: 285.15 [M+H]⁺.

### Synthesis of (2S,4R)-1-((S)-2-(4-cyclopropyl-1H-1,2,3-triazol-1-yl)-3,3-dimethylbutanoyl)-4-hydroxypyrrolidine-2-carboxylic acid, 8

A solution of 5 (700 mg, 2.46 mmol, 1.0 eq), 6 (488 mg, 7.39 mmol, 3.0 eq), sodium ascorbate (980 mg, 2.46 mmol, 2.0 eq), CuSO₄·10 (H₂O) (614 mg, 2.46 mmol, 1.0 eq) in t-BuOH/H₂O (50/25 mL) was stirred overnight. The reaction solution was concentrated. The mixture was redissolved into a mixture of EA/H₂O (80/80 mL), the organic phase was collected and washed by water once. After dried by anhydrous Na₂SO₄ and concentrated. The residue was received and redissolved into MeOH (40 mL). An aqueous solution of LiOH (1 N, 10 mL) was added, the mixture stirred for 2 hours. After reaction the solution was neutralized by HCl to pH to 2-3. After concentration, the residue was purified by C-18 reversal column chromatography (elution solvents: CH₃CN/H₂O from 10% to 100%, 0.1% TFA) to get target product 8 (600 mg, 72%) as a slight yellow solid. ESI MS m/z: 337.18 [M+H]⁺.

### Synthesis of (R)-2-azido-1-(4-(4-methylthiazol-5-yl)phenyl) ethan-1-amine, 12

To a solution of 9 (1.00 g, 2.99 mmol, 1.0 eq) in DCM (30 mL), was added TEA (605 mg, 5.98 mmol, 2 eq), MsCl (445 mg, 3.89 mmol, 1.3 eq) at 0° C. The mixture was stirred for 15 minutes. A saturated solution of NaHCO₃ (30 mL) was added to quench the reaction and the mixture was stirred for further 10 minutes. The DCM layer was collected. After dried by anhydrous Na₂SO₄ and concentrated. The received residue was dissolved into DMF (30 mL), NaN₃ (583 mg, 8.97 mmol, 3 eq) was added, the mixture stirred for 16 h at 80° C. After reaction, water was added to quench the reaction and product was extracted by EA twice. Combined EA phases were washed by water twice. After dried by anhydrous Na₂SO₄, the solution was concentrated. The residue was redissolved into DCM (20 mL), then TFA (10 mL) was added. The reaction was stirred for 30 minutes. After concentration, the residue was purified by C-18 reversal column chromatography (elution solvents: CH₃CN/H₂O from 10% to 100%, 0.1% TFA) to get target product 12 (500 mg, 71%) as a slight yellow solid. ESI MS m/z: 260.10 [M+H]⁺.

### Synthesis of (2S,4R)—N—((R)-2-azido-1-(4-(4-methylthiazol-5-yl)phenyl)ethyl)-1-((S)-2-(4-cyclopropyl-1H-1,2,3-triazol-1-yl)-3,3-dimethylbutanoyl)-4-hydroxypyrrolidine-2-carboxamide, 15

To suspension of 13 (1.0 g, 2.97 mmol, 1 eq) in DMF (30 mL) was added 14 (770 mg, 2.97 mmol, 1.0 eq), HATU (1.70 g, 4.46 mmol, 1.5 eq), DIPEA (1.15 g, 8.92 mmol, 3.0 eq). The mixture was stirred for 30 minutes. The reaction was quenched by added water. The mixture was extracted by ethyl acetate (80*2 mL) twice. Combined organic phases were washed by water twice. After dried by anhydrous Na₂SO₄ and concentrated. The residue was purified by C-18 reversal column chromatography (elution solvents: CH₃CN/H₂O from 10% to 100%, 0.1% TFA) to get target product 15 (1.36 g, 79%) as a slight yellow oil. ESI MS m/z: 758.26 [M+H]⁺.

### Synthesis of (2S,4R)—N—((R)-2-(4-((4-(4′-bromo-5′-oxo-5′H-spiro[cyclohexane-1,7′-indolo[1,2-a]quinazolin]-10′-yl)piperidin-1-yl)methyl)-1H-1,2,3-triazol-1-yl)-1-(4-(4-methylthiazol-5-yl)phenyl)ethyl)-1-((S)-2-(4-cyclopropyl-1H-1,2,3-triazol-1-yl)-3,3-dimethylbutanoyl)-4-hydroxypyrrolidine-2-carboxamide, A114

A solution of 15 (50 mg, 0.086 mmol, 1.0 eq), 16 (43 mg, 0.086 mmol, 1.0 eq), sodium ascorbate (34 mg, 0.17 mmol, 2.0 eq), CuSO₄·10 (H₂O) (21 mg, 0.086 mmol, 1.0 eq) in t-BuOH/H₂O (20/10 mL) was stirred overnight. The reaction solution was concentrated. After concentration, the residue was purified by pre-HPLC (elution solvents: CH₃CN/H₂O from 10% to 100%, 0.1% TFA) to get target product 17 (56 mg, 60%) as a slight yellow solid. ESI MS m/z: 1081.40 [M+H]⁺.

### Compound A115. (2S,4R)—N—((R)-2-(4-((4-(4′-bromo-5′-oxo-5′H-spiro[cyclohexane-1,7′-indolo[1,2-a]quinazolin]-9′-yl)piperidin-1-yl)methyl)-1H-1,2,3-triazol-1-yl)-1-(4-(4-methylthiazol-5-yl)phenyl)ethyl)-1-((S)-2-(4-cyclopropyl-1H-1,2,3-triazol-1-yl)-3,3-dimethylbutanoyl)-4-hydroxypyrrolidine-2-carboxamide, 3

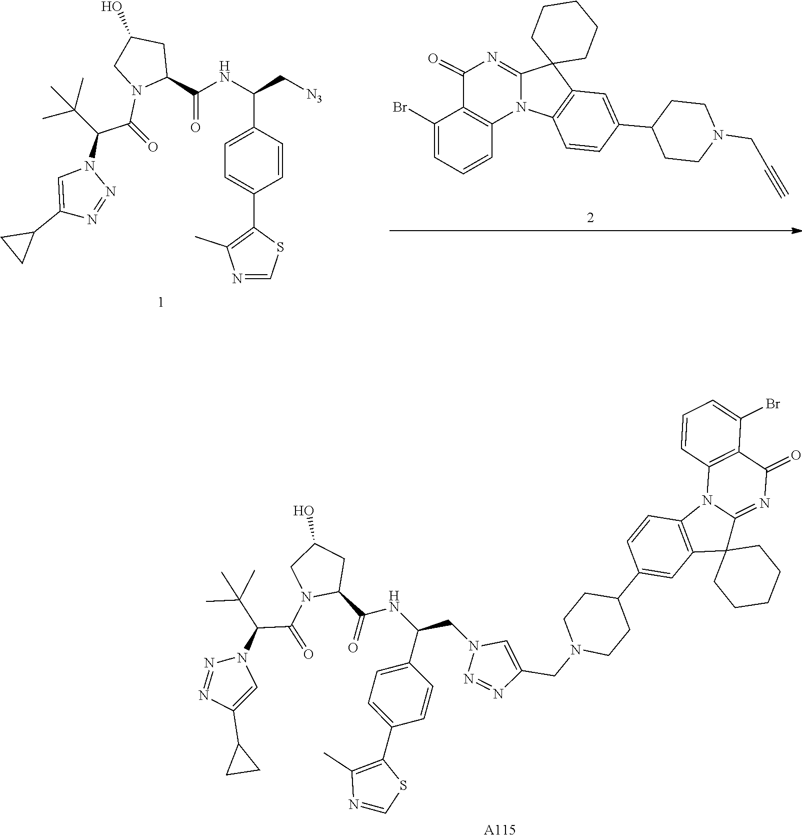 [SMILES: Cc1ncsc1-c1ccc([C@H](CN=[N+]=[N-])NC(=O)[C@@H]2C[C@@H](O)CN2C(=O)[C@@H](n2cc(C3CC3)nn2)C(C)(C)C)cc1 ; C#CCN1CCC(c2ccc3c(c2)C2(CCCCC2)c2nc(=O)c4c(Br)cccc4n2-3)CC1 ; Cc1ncsc1-c1ccc([C@H](Cn2cc(CN3CCC(c4ccc5c(c4)C4(CCCCC4)c4nc(=O)c6c(Br)cccc6n4-5)CC3)nn2)NC(=O)[C@@H]2C[C@@H](O)CN2C(=O)[C@@H](n2cc(C3CC3)nn2)C(C)(C)C)cc1 ; "A115"]

A solution of 1 (50 mg, 0.086 mmol, 1.0 eq), 2 (43 mg, 0.086 mmol, 1.0 eq), sodium ascorbate (34 mg, 0.17 mmol, 2.0 eq), CuSO₄·10 (H₂O) (21 mg, 0.086 mmol, 1.0 eq) in t-BuOH/H₂O (20/10 mL) was stirred overnight. The reaction solution was concentrated. After concentration, the residue was purified by pre-HPLC (elution solvents: CH₃CN/H₂O from 10% to 100%, 0.1% TFA) to get target product 3 (63 mg, 67%) as a slight yellow solid. ESI MS m/z: 1081.40 [M+H]⁺.

### Compound A116. (2S,4R)—N—((R)-2-((1r,4R)-4-((4-(4′-bromo-5′-oxo-5′H-spiro[cyclohexane-1,7′-indolo[1,2-a]quinazolin]-10′-yl)piperidin-1-yl)methyl)cyclohexane-1-carboxamido)-1-(4-(4-methylthiazol-5-yl)phenyl)ethyl)-1-((S)-2-(4-cyclopropyl-1H-1,2,3-triazol-1-yl)-3,3-dimethylbutanoyl)-4-hydroxypyrrolidine-2-carboxamide

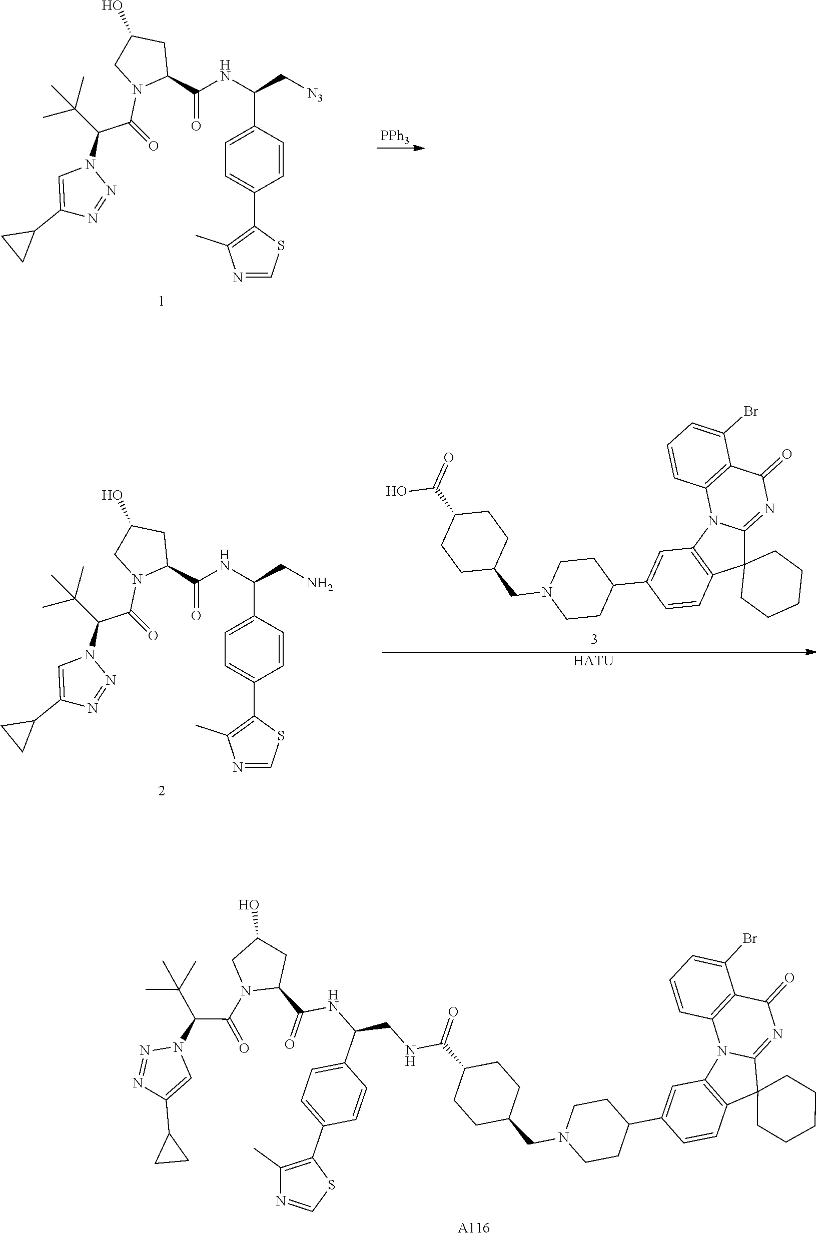

### Synthesis of (2S,4R)—N—((R)-2-amino-1-(4-(4-methylthiazol-5-yl)phenyl)ethyl)-1-((S)-2-(4-cyclopropyl-1H-1,2,3-triazol-1-yl)-3,3-dimethylbutanoyl)-4-hydroxypyrrolidine-2-carboxamide

To a solution of 1 (300 mg, 0.52 mmol, 1 eq) in THF/H₂O (20/4 mL) was added PPh₃ (204 mg, 0.78 mmol, 1.5 eq). The mixture was stirred for 3 h at 60° C. After reaction, the solution was concentrated and the residue was purified by C-18 reversal column chromatography (elution solvents: CH₃CN/H₂O from 10% to 100%, 0.1% TFA) to get target product 2 (230 mg, 80%) as a slight yellow solid. ESI MS m/z: 552.27 [M+H]⁺.

### Step 2

To suspension of 2 (30 mg, 0.054 mmol, 1 eq) in DMF (3 mL) was added 3 (33 mg, 0.054 mmol, 1.0 eq), HATU (31 mg, 0.082 mmol, 1.5 eq), DIPEA (21 mg, 0.16 mmol, 3.0 eq). The mixture was stirred for 30 minutes. The mixture was purified by pre-HPLC (elution solvents: CH₃CN/H₂O from 10% to 100%, 0.1% TFA) to get target product 4 (40 mg, 65%) as a slight yellow solid. ESI MS m/z: 1139.46 [M+H]⁺.

### Compound A117. (2S,4R)—N—((R)-2-((1r,4R)-4-((4-(4′-bromo-5′-oxo-5′H-spiro[cyclohexane-1,7′-indolo[1,2-a]quinazolin]-9′-yl)piperidin-1-yl)methyl)cyclohexane-1-carboxamido)-1-(4-(4-methylthiazol-5-yl)phenyl)ethyl)-1-((S)-2-(4-cyclopropyl-1H-1,2,3-triazol-1-yl)-3,3-dimethylbutanoyl)-4-hydroxypyrrolidine-2-carboxamide

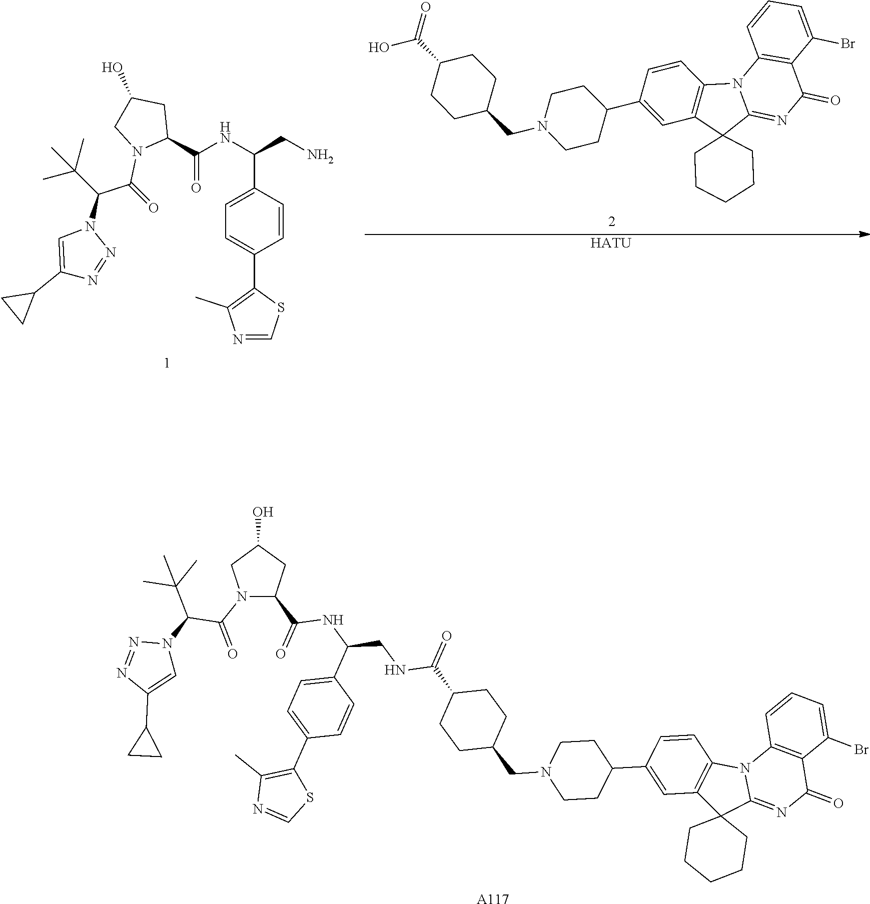 [SMILES: Cc1ncsc1-c1ccc([C@H](CN)NC(=O)[C@@H]2C[C@@H](O)CN2C(=O)[C@@H](n2cc(C3CC3)nn2)C(C)(C)C)cc1 ; O=c1nc2n(c3cccc(Br)c13)-c1ccc(C3CCN(C[C@H]4CC[C@H](C(=O)O)CC4)CC3)cc1C21CCCCC1 ; "HATU" ; Cc1ncsc1-c1ccc([C@H](CNC(=O)[C@H]2CC[C@H](CN3CCC(c4ccc5c(c4)C4(CCCCC4)c4nc(=O)c6c(Br)cccc6n4-5)CC3)CC2)NC(=O)[C@@H]2C[C@@H](O)CN2C(=O)[C@@H](n2cc(C3CC3)nn2)C(C)(C)C)cc1 ; "A117"]

The procedure is same to the former compound A116. Target compound was received. ESI MS m/z: 1139.46 [M+H]⁺.

### Compound A118. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-cyclohexylacetyl)-4-hydroxy-N—((S)-1-(4-(4-methylthiazol-5-yl)phenyl)ethyl)pyrrolidine-2-carboxamide

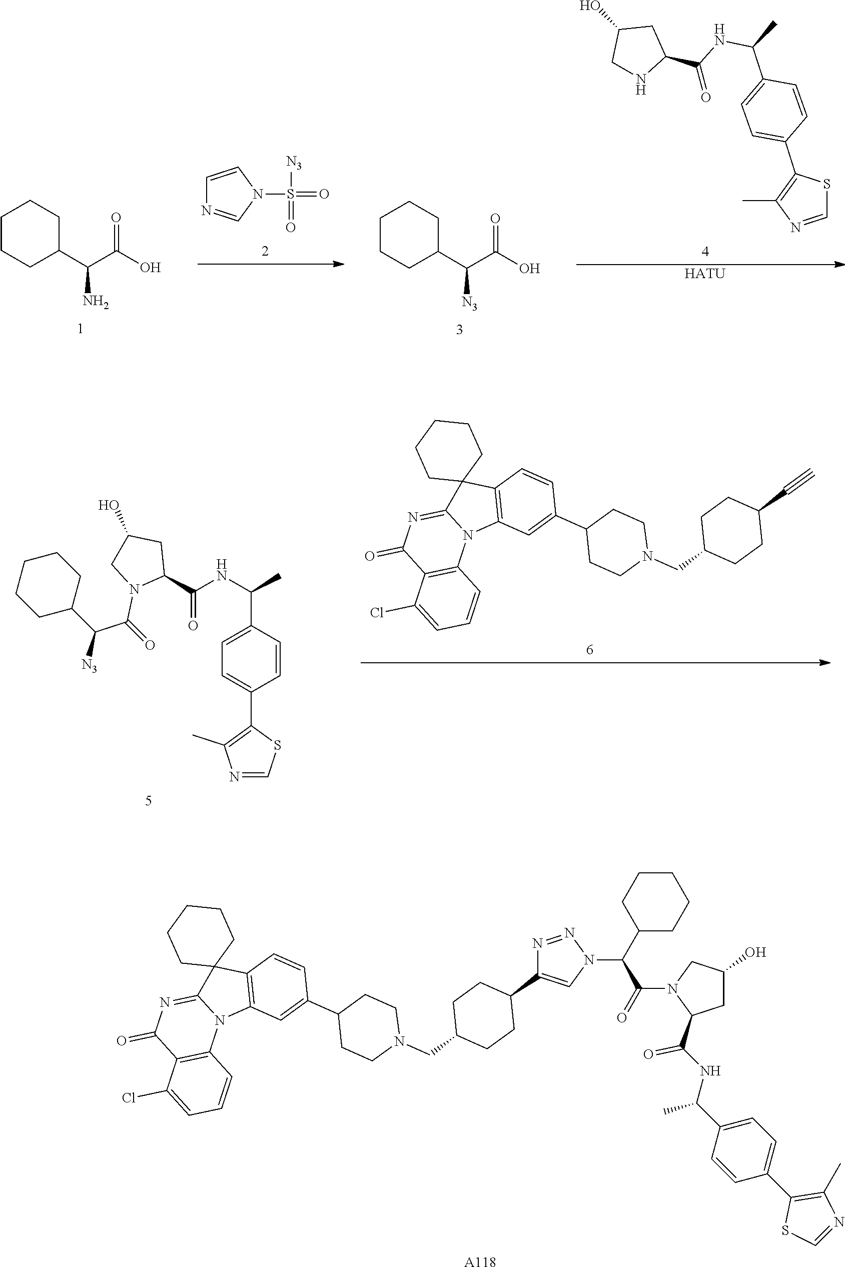

The reaction routes showed as above. The procedure is similar as former procedure. Target degrader was obtained as a white solid. ESI MS m/z: 1036.50 [M+H]⁺.

### Compound A119. (2S,4R)-1-((S)-2-(4-((1r,4S)-4-((4-(4′-bromo-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-cyclohexylacetyl)-4-hydroxy-N—((S)-1-(4-(4-methylthiazol-5-yl)phenyl)ethyl)pyrrolidine-2-carboxamide

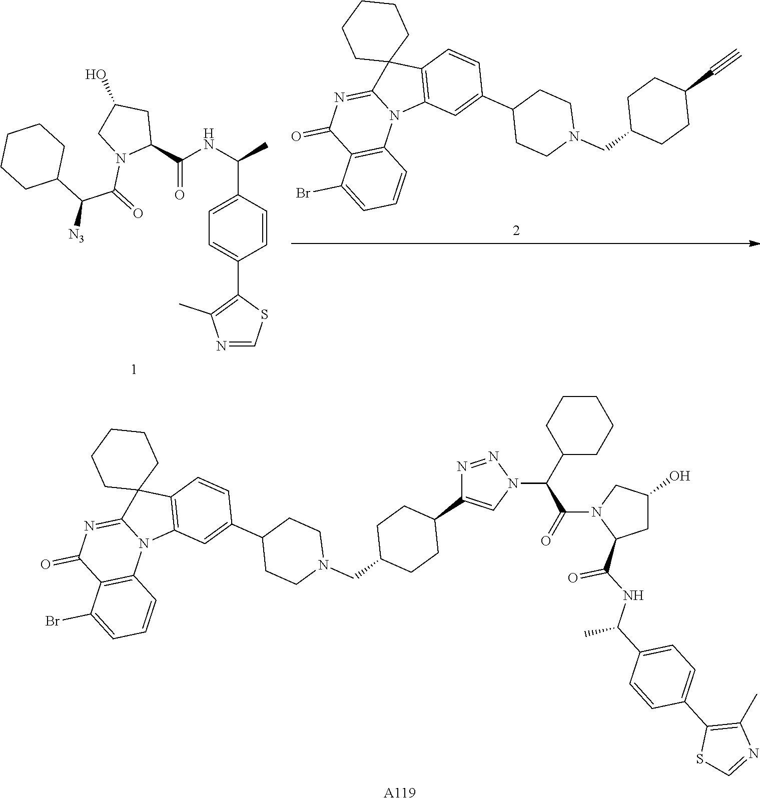

The reaction routes showed as above. The procedure is similar as former procedure. Target degrader was obtained as a white solid. ESI MS m/z: 1082.44 [M+H]⁺.

### Compound A120. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-(tetrahydro-2H-pyran-4-yl) acetyl)-4-hydroxy-N—((S)-1-(4-(4-methylthiazol-5-yl)phenyl)ethyl)pyrrolidine-2-carboxamide

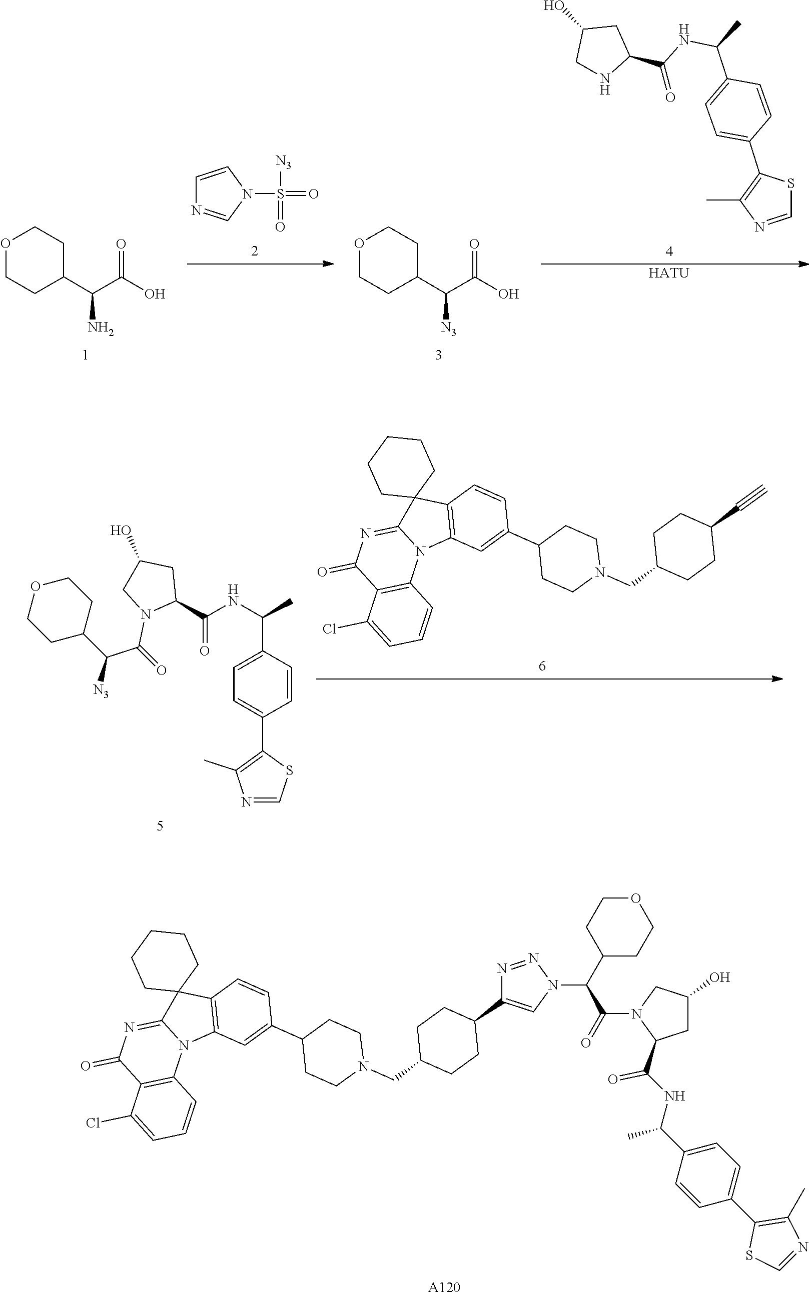

The reaction routes showed as above. The procedure is similar as former procedure. Target degrader was obtained as a white solid. ESI MS m/z: 1038.48 [M+H]⁺.

### Compound A121. (2S,4R)-1-((S)-2-(4-((1r,4S)-4-((4-(4′-bromo-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-(tetrahydro-2H-pyran-4-yl) acetyl)-4-hydroxy-N—((S)-1-(4-(4-methylthiazol-5-yl)phenyl)ethyl)pyrrolidine-2-carboxamide

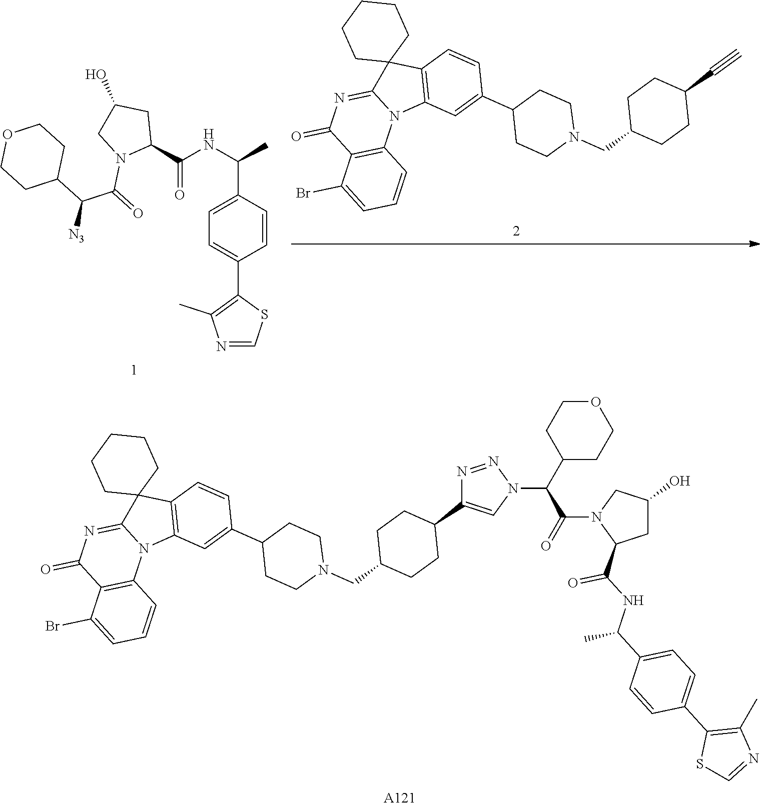

The reaction routes showed as above. The procedure is similar as former procedure. Target degrader was obtained as a white solid. ESI MS m/z: 1084.42 [M+H]⁺.

### Compound A122. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-cyclohexylacetyl)-4-hydroxy-N—((R)-1-(4-(4-methylthiazol-5-yl)phenyl)-2-morpholinoethyl)pyrrolidine-2-carboxamide

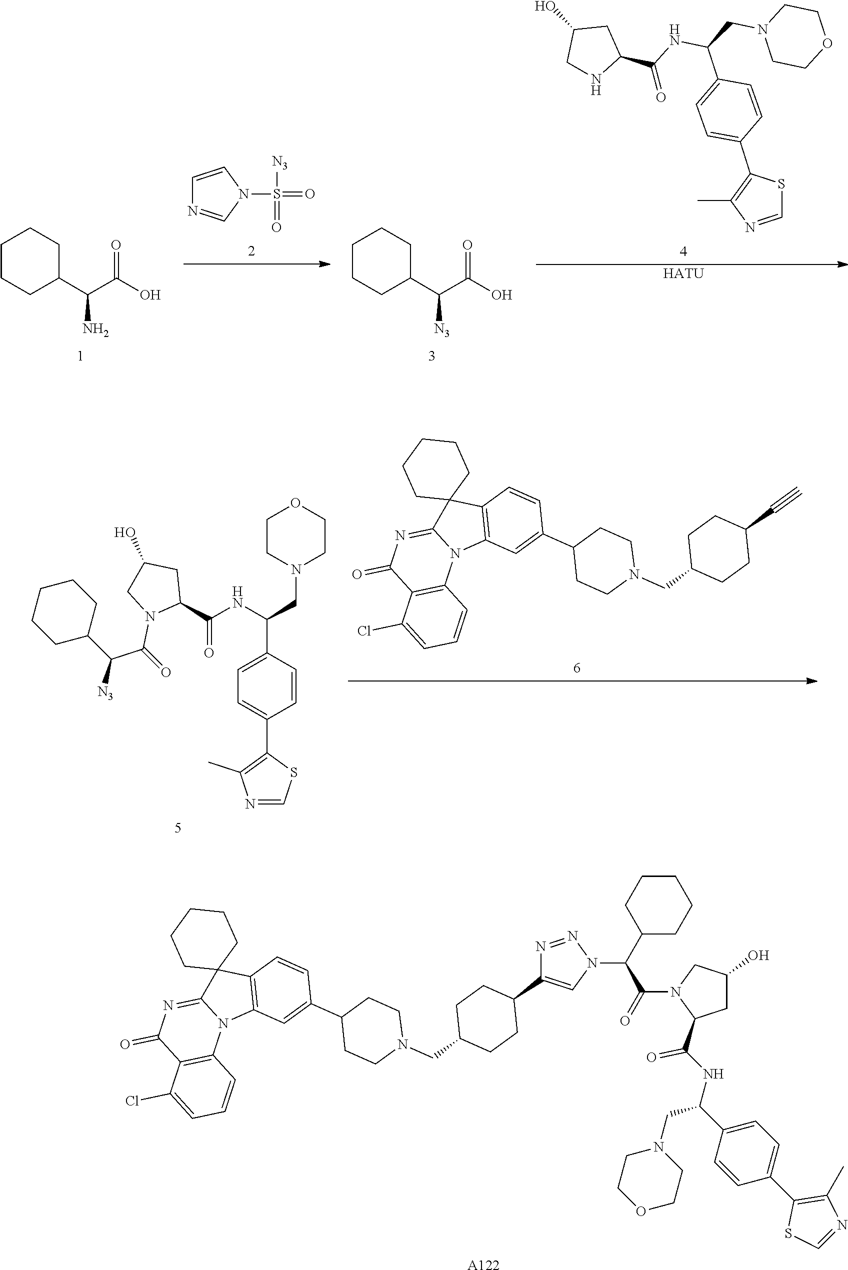

The reaction routes showed as above. The procedure is similar as former procedure. Target degrader was obtained as a white solid. ESI MS m/z: 1121.55 [M+H]⁺.

### Compound A123. (2S,4R)-1-((S)-2-(4-((1r,4S)-4-((4-(4′-bromo-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-cyclohexylacetyl)-4-hydroxy-N—((R)-1-(4-(4-methylthiazol-5-yl)phenyl)-2-morpholinoethyl)pyrrolidine-2-carboxamide

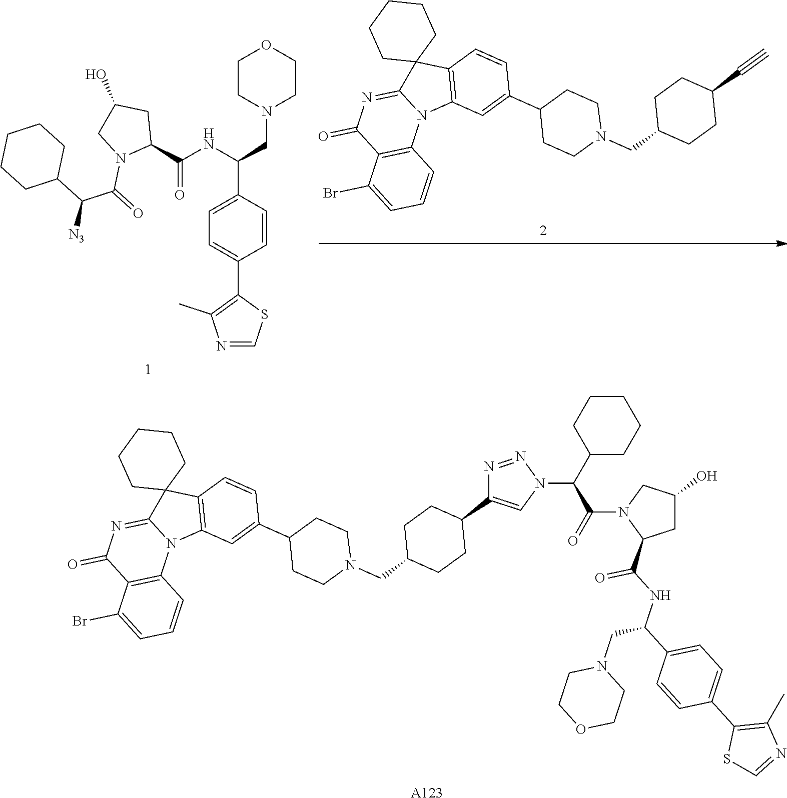 [SMILES: Cc1ncsc1-c1ccc([C@H](CN2CCOCC2)NC(=O)[C@@H]2C[C@@H](O)CN2C(=O)[C@@H](N=[N+]=[N-])C2CCCCC2)cc1 ; C#C[C@H]1CC[C@H](CN2CCC(c3ccc4c(c3)-n3c(nc(=O)c5c(Br)cccc53)C43CCCCC3)CC2)CC1 ; Cc1ncsc1-c1ccc([C@H](CN2CCOCC2)NC(=O)[C@@H]2C[C@@H](O)CN2C(=O)[C@H](C2CCCCC2)n2cc([C@H]3CC[C@H](CN4CCC(c5ccc6c(c5)-n5c(nc(=O)c7c(Br)cccc75)C65CCCCC5)CC4)CC3)nn2)cc1 ; "A123"]

The reaction routes showed as above. The procedure is similar as former procedure. Target degrader was obtained as a white solid. ESI MS m/z: 1167.50 [M+H]⁺.

### Compound A124. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-2-cyclohexylacetyl)-4-hydroxy-N—((S)-1-(4-(4-methylthiazol-5-yl)phenyl)ethyl)pyrrolidine-2-carboxamide

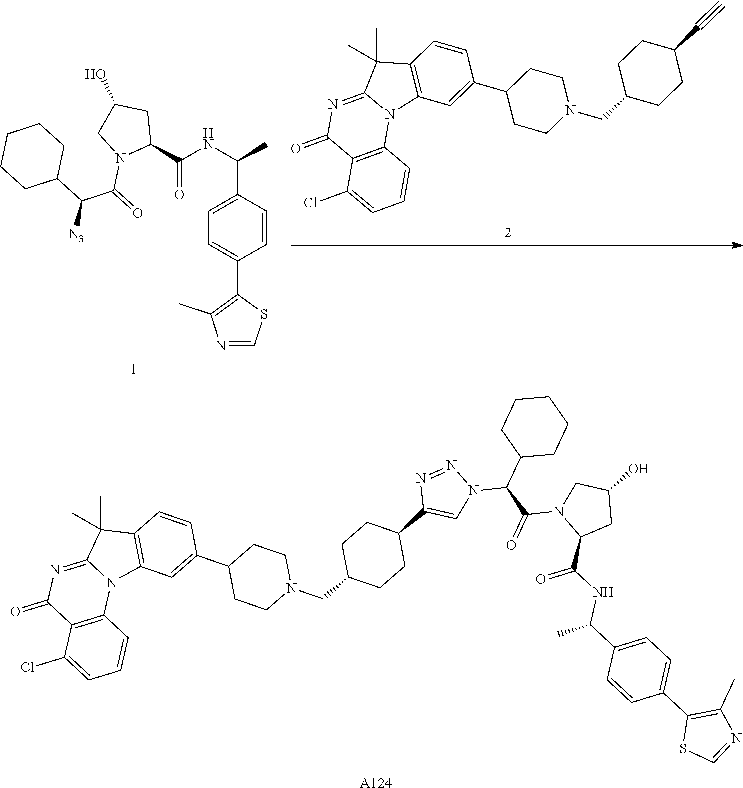

The reaction routes showed as above. The procedure is similar as former procedure. Target degrader was obtained as a white solid. ESI MS m/z: 996.46 [M+H]⁺.

### Compound A125. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-2-cyclohexylacetyl)-4-hydroxy-N—((R)-1-(4-(4-methylthiazol-5-yl)phenyl)-2-morpholinoethyl)pyrrolidine-2-carboxamide

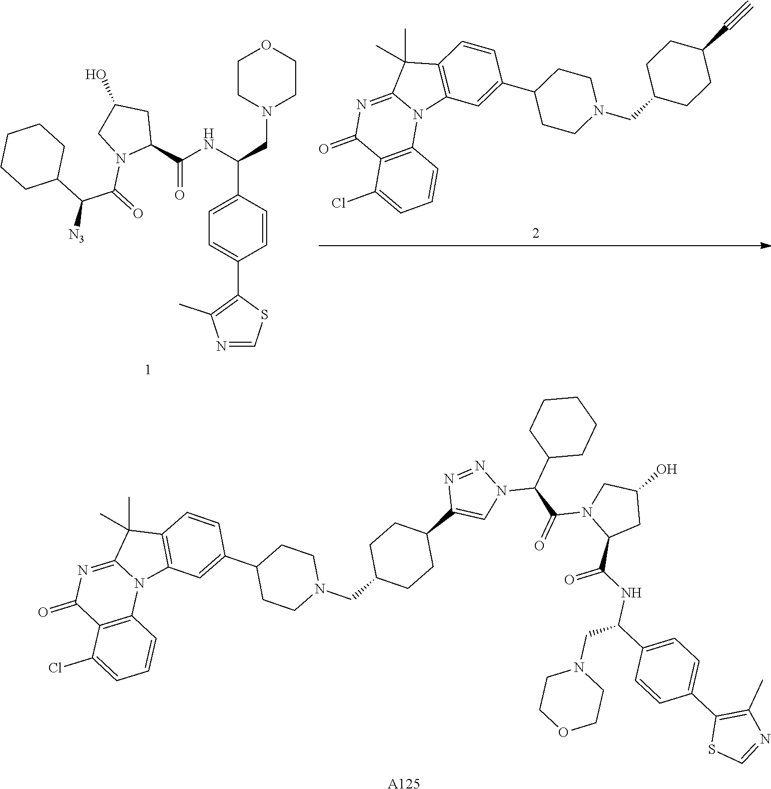 [SMILES: Cc1ncsc1-c1ccc([C@H](CN2CCOCC2)NC(=O)[C@@H]2C[C@@H](O)CN2C(=O)[C@@H](N=[N+]=[N-])C2CCCCC2)cc1 ; C#C[C@H]1CC[C@H](CN2CCC(c3ccc4c(c3)-n3c(nc(=O)c5c(Cl)cccc53)C4(C)C)CC2)CC1 ; Cc1ncsc1-c1ccc([C@H](CN2CCOCC2)NC(=O)[C@@H]2C[C@@H](O)CN2C(=O)[C@H](C2CCCCC2)n2cc([C@H]3CC[C@H](CN4CCC(c5ccc6c(c5)-n5c(nc(=O)c7c(Cl)cccc75)C6(C)C)CC4)CC3)nn2)cc1 ; "A125"]

The reaction routes showed as above. The procedure is similar as former procedure. Target degrader was obtained as a white solid. ESI MS m/z: 1081.52 [M+H]⁺.

### Compound A126. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-cyclohexylacetyl)-4-hydroxy-N—((R)-2-hydroxy-1-(4-(4-methylthiazol-5-yl)phenyl)ethyl)pyrrolidine-2-carboxamide

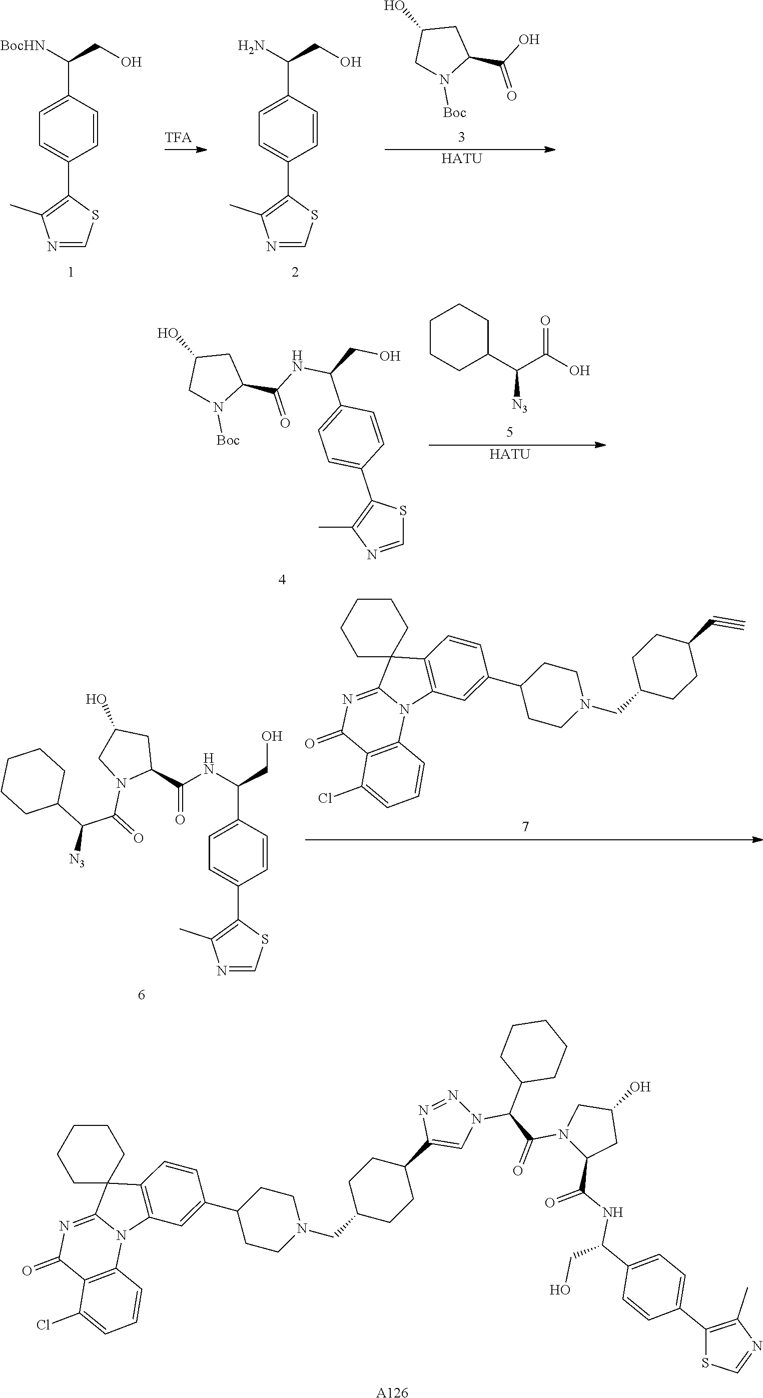

The reaction routes showed as above. The procedure is similar as former procedure. Target degrader was obtained as a white solid. ESI MS m/z: 1052.49 [M+H]⁺.

### Compound A127. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-cyclohexylacetyl)-4-hydroxy-N—((R)-2-hydroxy-1-(4-(4-methylthiazol-5-yl)phenyl)ethyl)pyrrolidine-2-carboxamide

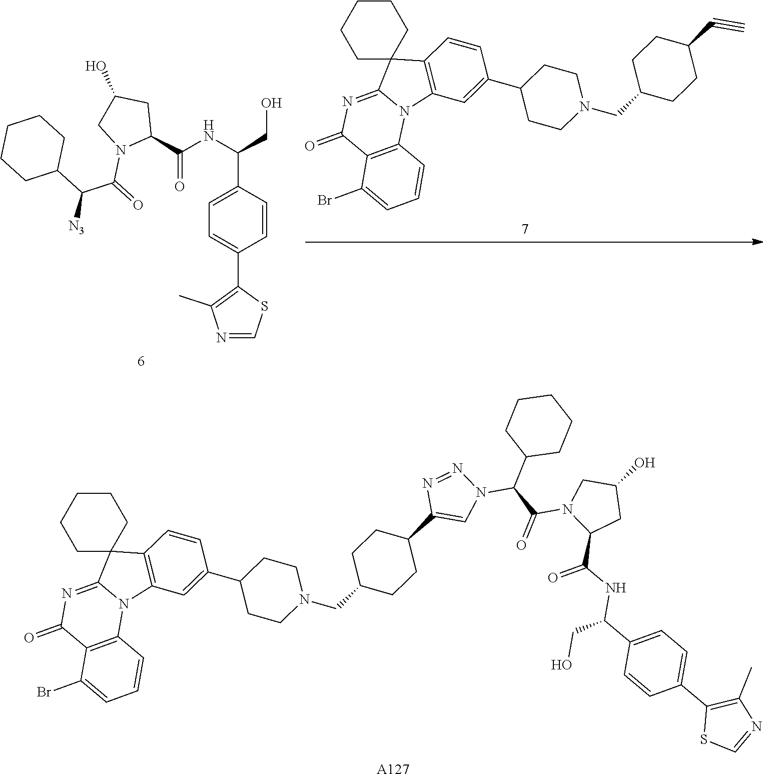

The reaction routes showed as above. The procedure is similar as former procedure. Target degrader was obtained as a white solid. ESI MS m/z: 1098.44 [M+H]⁺.

### Compound A128. (2S,4R)-1-((S)-2-(4-(4-((4-(4′-bromo-5′-oxo-5′H-spiro[cyclohexane-1,7′-indolo[1,2-a]quinazolin]-10′-yl)piperidin-1-yl)methyl)phenyl)-1H-1,2,3-triazol-1-yl)-2-cyclohexylacetyl)-4-hydroxy-N—((R)-1-(4-(4-methylthiazol-5-yl)phenyl)-2-morpholinoethyl)pyrrolidine-2-carboxamide

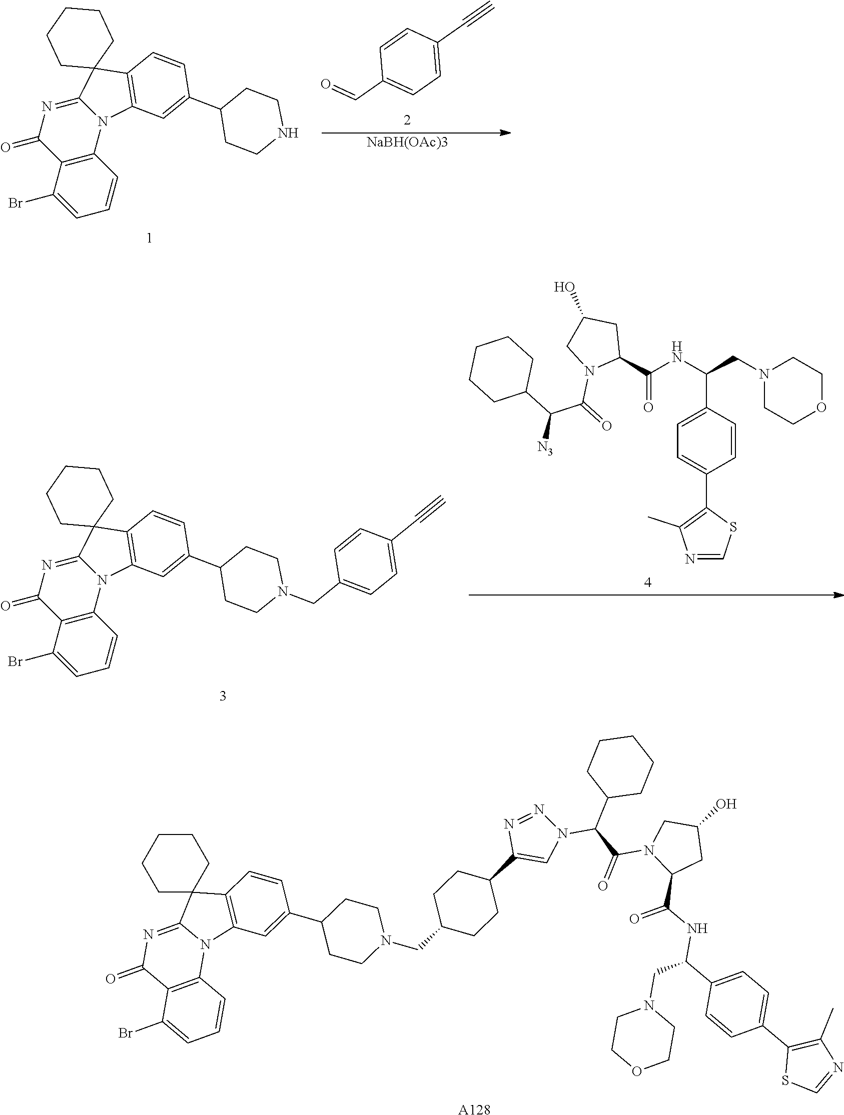

The reaction routes showed as above. The procedure is similar as former procedure. Target degrader was obtained as a white solid. ESI MS m/z: 1165.44 [M+H]⁺.

### Compound A129. (2S,4R)-1-((S)-2-(4-(4-((4-(4′-bromo-5′-oxo-5′H-spiro[cyclohexane-1,7′-indolo[1,2-a]quinazolin]-10′-yl)piperidin-1-yl)methyl)phenyl)-1H-1,2,3-triazol-1-yl)-2-cyclohexylacetyl)-4-hydroxy-N—((R)-2-hydroxy-1-(4-(4-methylthiazol-5-morpholinoethyl)pyrrolidine-2-carboxamide

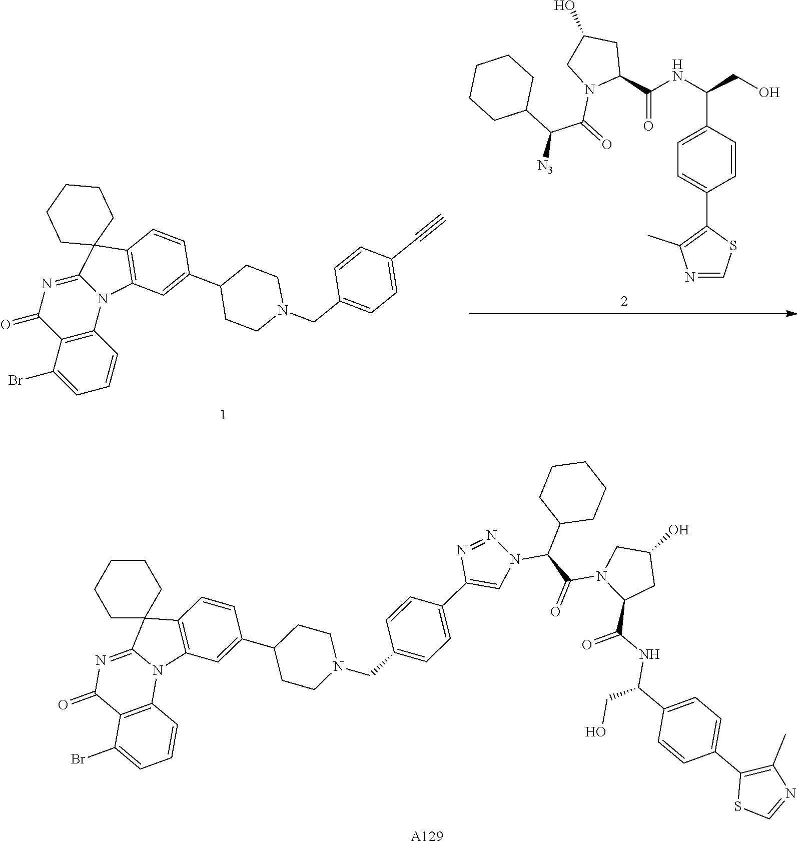

The reaction routes showed as above. The procedure is similar as former procedure. Target degrader was obtained as a white solid. ESI MS m/z: 1039.44 [M+H]⁺.

### Compound A130. (2S,4R)-1-((2S)-2-(4-((1r,4S)-4-((3-(4′-chloro-5′-oxo-5′H-spiro[cyclohexane-1,7′-indolo[1,2-a]quinazolin]-10′-yl) pyrrolidin-1-yl)methyl)cyclohexyl)-1H-1,2,3-triazol-1-yl)-2-cyclohexylacetyl)-4-hydroxy-N—((R)-1-(4-(4-methylthiazol-5-yl)phenyl)-2-morpholinoethyl)pyrrolidine-2-carboxamide

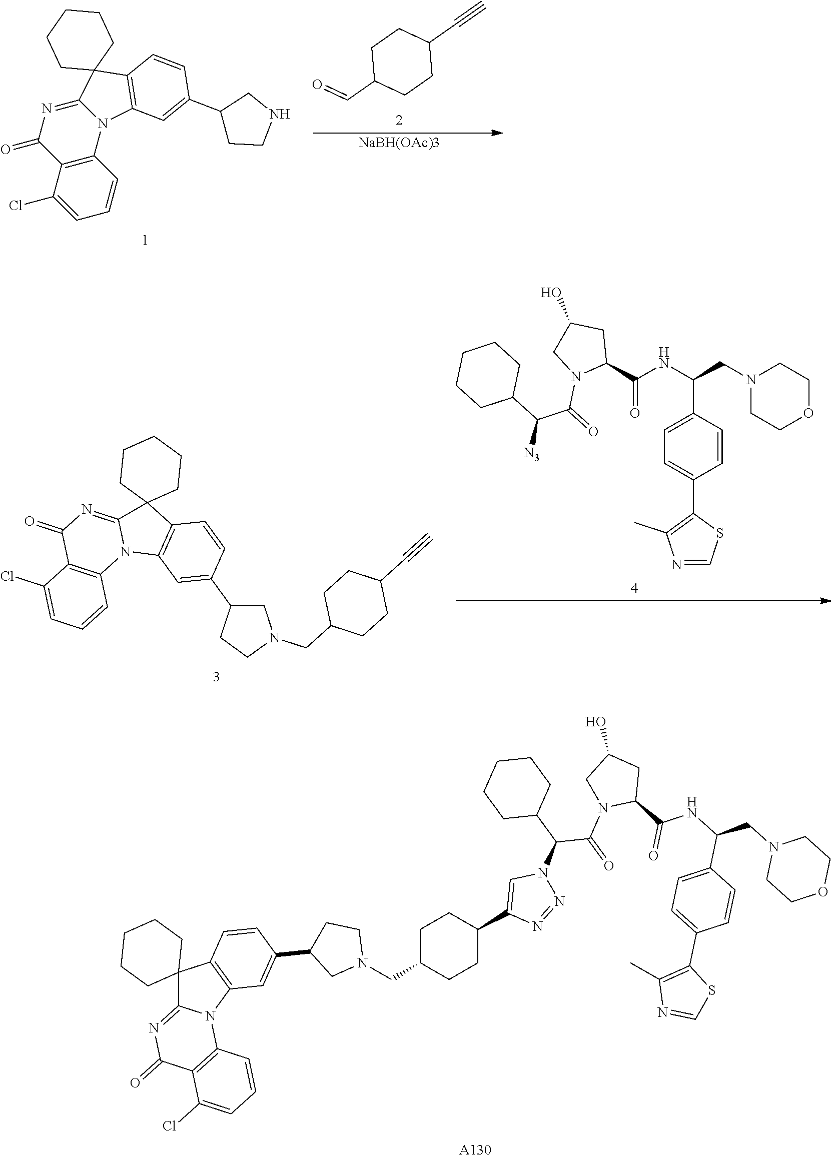

The reaction routes showed as above. The procedure is similar as former procedure. Target degrader was obtained as a white solid. ESI MS m/z: 1107.54 [M+H]⁺.

### Compound A131. (2S,4R)-1-((2S)-2-(4-((1r,4S)-4-((3-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-cyclohexylacetyl)-4-hydroxy-N—((R)-1-(4-(4-methylthiazol-5-yl)phenyl)-2-morpholinoethyl)pyrrolidine-2-carboxamide

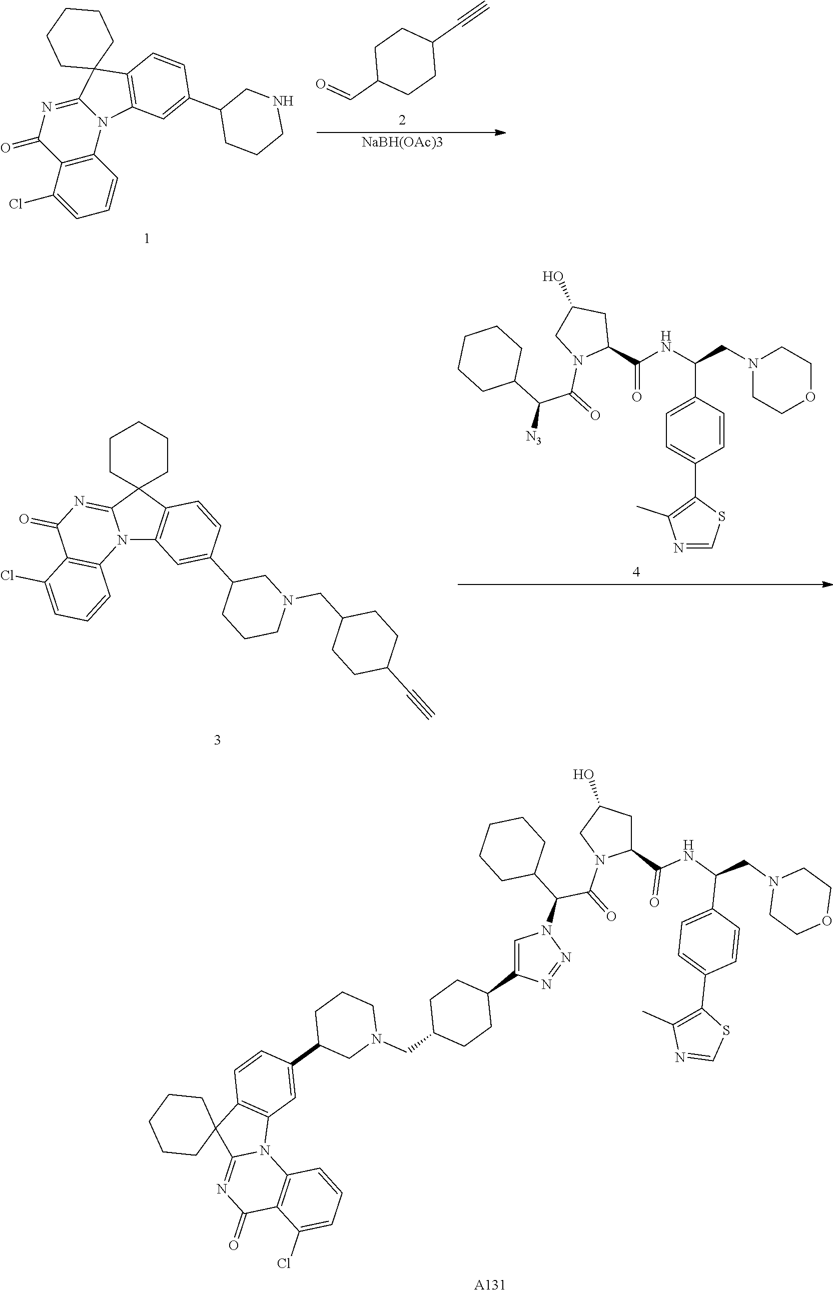

The reaction routes showed as above. The procedure is similar as former procedure. Target degrader was obtained as a white solid. ESI MS m/z: 1121.55 [M+H]⁺.

### Compound A132. (2S,4R)-1-((S)-2-(1-((1r,4S)-4-((4-(4′-bromo-5′-oxo-5′H-spiro[cyclohexane-1,7′-indolo[1,2-a]quinazolin]-10′-yl)piperidin-1-yl)methyl)cyclohexane-1-carbonyl)piperidin-4-yl)-2-(4-cyclopropyl-1H-1,2,3-triazol-1-yl) acetyl)-4-hydroxy-N—((S)-1-(4-(4-methylthiazol-5-yl)phenyl)ethyl)pyrrolidine-2-carboxamide

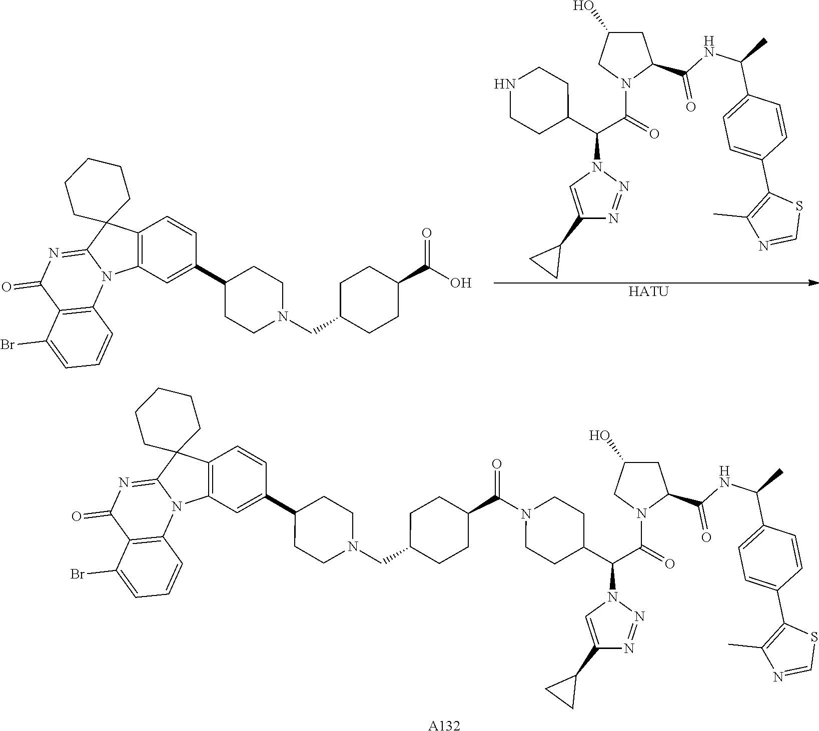

The reaction routes showed as above. The procedure is similar as former procedure. Target degrader was obtained as a white solid. ESI MS m/z: 1151.45 [M+H]⁺.

### Compound A133. 5-(4-((R)-1-((2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-2-(tetrahydro-2H-pyran-4-yl) acetyl)-4-hydroxypyrrolidine-2-carboxamido)-2-morpholinoethyl)phenyl)thiazole-4-carboxylic acid

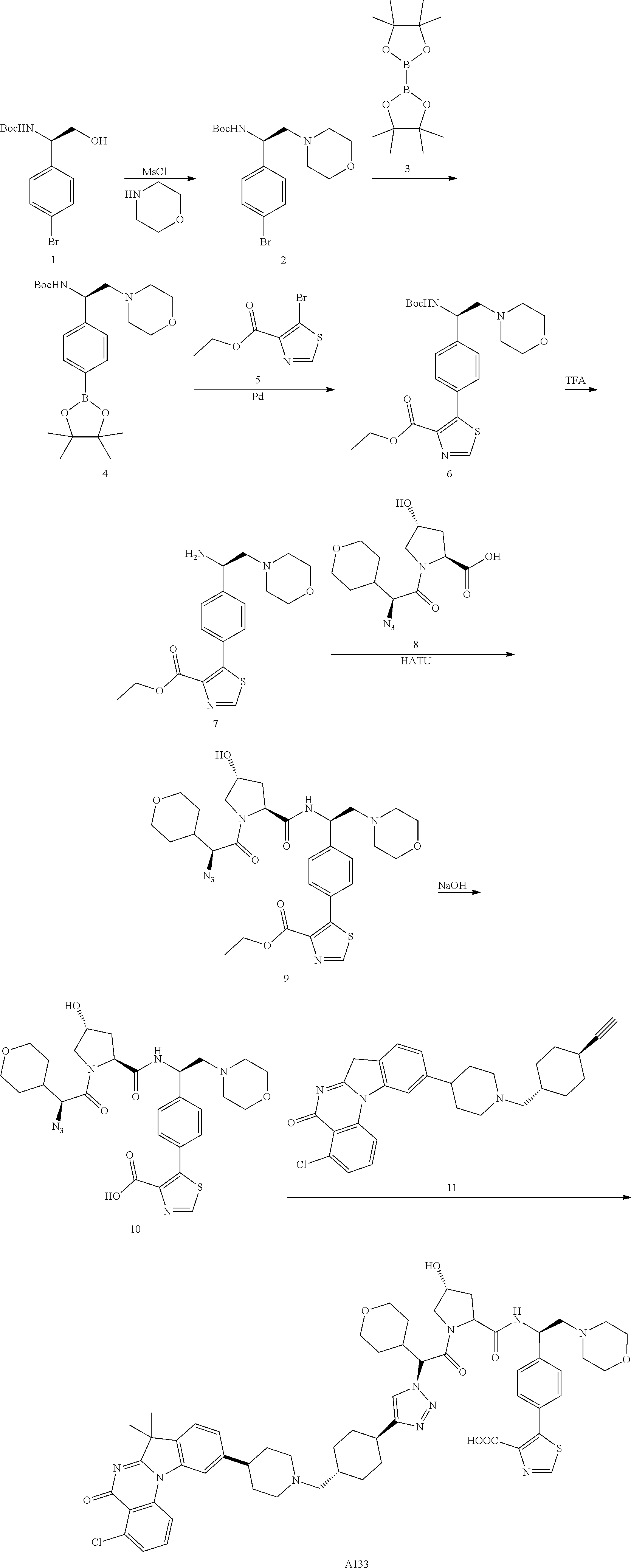

### Synthesis of tert-butyl (R)-(1-(4-bromophenyl)-2-morpholinoethyl)carbamate, 2

To a solution of 1 (5.00 g, 15.8 mmol, 1.0 eq) in DCM (100 mL), was added TEA (4.8 g, 47.44 mmol, 3 eq), MsCl (2.35 g, 20.56 mmol, 1.3 eq) at 0° C. The mixture was stirred for 15 minutes. A saturated solution of NaHCO₃ (80 mL) was added to quench the reaction and the mixture was stirred for further 10 minutes. The DCM layer was collected. After dried by anhydrous Na₂SO₄ and concentrated. The received residue was dissolved into morpholine (80 mL), the mixture stirred for 16 h at 90° C. After reaction, morpholine was removed under reduce pressure evaporation. The residue was dissolved into a mixture of EA/water. The organic layer was collected, and water was extracted by EA once. Combined EA phases were washed by water once. After dried by anhydrous Na₂SO₄, the solution was concentrated. The residue was purified by silica gel column chromatography (elution solvents: EA/hexane=1/1, v/v) to get target product 2 (3.2 g, 53%) as a slight yellow solid. ESI MS m/z: 385.10 [M+H]⁺.

### Synthesis of tert-butyl (R)-(2-morpholino-1-(4-(4,4,5,5-tetramethyl-1,3,2-dioxaborolan-2-yl)phenyl)ethyl)carbamate, 4

To a solution of 2 (2.00 g, 5.19 mmol, 1.0 eq) in DMSO (80 mL), was added Pd(dppf)Cl₂ (380 mg, 0.52 mmol, 0.1 eq), 3 (1.71 g, 6.75 mmol, 1.3 eq) and AcOK (1.02 g, 10.38 mmol, 2 eq. The mixture was stirred for 3 h at 80° C. Water (200 mL) was added to quench the reaction. The mixture was extracted by EA twice. The combined organic layers were washed by water twice. After dried by anhydrous Na₂SO₄ and concentrated. The residue was purified by silica gel column chromatography (elution solvents: EA/hexane=1/1, v/v) to get target product 4 (1.6 g, 71%) as a slight yellow solid. ESI MS m/z: 433.28 [M+H]⁺.

### Synthesis of ethyl (R)-5-(4-(1-((tert-butoxycarbonyl)amino)-2-morpholinoethyl)phenyl)thiazole-4-carboxylate 6

To a solution of 4 (1.2 g, 2.78 mmol, 1.0 eq) in dioxane (80/20 mL), was added Pd(dppf)Cl₂ (203 mg, 0.28 mmol, 0.1 eq), 5 (786 mg, 3.33 mmol, 1.2 eq) and K₂CO₃ (767 mg, 5.55 mmol, 2 eq). The mixture was stirred for 3 h at 90° C. After reaction, the solvent was removed. The residue was dissolved into a mixture of EA/H₂O. The organic phase was collected. After dried by anhydrous Na₂SO₄ and concentrated. The residue was purified by silica gel column chromatography (elution solvents: EA/hexane=1/1, v/v) to get target product 2 (860 mg, 67%) as a slight yellow solid. ESI MS m/z: 462.20 [M+H]⁺.

### Synthesis of ethyl 5-(4-((R)-1-((2S,4R)-1-((S)-2-azido-2-(tetrahydro-2H-pyran-4-yl) acetyl)-4-hydroxypyrrolidine-2-carboxamido)-2-morpholinoethyl)phenyl)thiazole-4-carboxylate 9

To a solution of 6 (850 mg, 1.84 mmol, 1.0 eq), in DCM (20 mL) was added TFA (10 mL) was added, the mixture stirred for 30 minutes. After reaction the solution was concentrated. The residue was dissolved in DMF (10 mL), 8 (550 mg, 1.84 mmol, 1.0 eq), HATU (1.05 g, 2.76 mmol, 1.5 eq), was added DIPEA (714 mg, 5.52 mmol, 3.0 eq). The mixture was stirred for 30 minutes. The reaction was quenched by added water. The mixture was extracted by ethyl acetate (60*2 mL) twice. Combined organic phases were washed by water twice. After dried by anhydrous Na₂SO₄ and concentrated. The residue was purified by C-18 reversal column chromatography (elution solvents: CH₃CN/H₂O from 10% to 100%, 0.1% TFA) to get target product 9 (830 mg, 70%) as a slight yellow solid. ESI MS m/z: 642.26 [M+H]⁺.

### Synthesis of 5-(4-((R)-1-((2S,4R)-1-((S)-2-azido-2-(tetrahydro-2H-pyran-4-yl) acetyl)-4-hydroxypyrrolidine-2-carboxamido)-2-morpholinoethyl)phenyl)thiazole-4-carboxylic acid, 10

To a solution of 9 in MeOH (10 mL) was added an aqueous solution of NaOH (1 N, 4 mL). The mixture was stirred for 1 h at rt. After reaction, HCl was added to neutralize the mixture until pH reach 2-3. The reaction was concentrated and purified by C-18 reversal column chromatography (elution solvents: CH₃CN/H₂O from 10% to 100%, 0.1% TFA) to get target product 10 (750 mg, 94%) as a slight yellow solid. ESI MS m/z: 614.23 [M+H]⁺.

### Synthesis of 5-(4-((R)-1-((2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-2-(tetrahydro-2H-pyran-4-yl) acetyl)-4-hydroxypyrrolidine-2-carboxamido)-2-morpholinoethyl)phenyl)thiazole-4-carboxylic acid. A133

A solution of 10 (30 mg, 0.048 mmol, 1.0 eq), 11 (25 mg, 0.048 mmol, 1.0 eq), sodium ascorbate (20 mg, 0.1 mmol, 2.0 eq), CuSO₄·10 (H₂O) (12.5 mg, 0.05 mmol, 1.0 eq) in t-BuOH/H₂O (20/10 mL) was stirred overnight. The reaction solution was concentrated. The residue was purified by pre-HPLC (elution solvents: CH₃CN/H₂O from 10% to 100%, 0.1% TFA) to get target product 12 (30 mg, 55%) as a slight yellow solid. ESI MS m/z: 1113.48 [M+H]⁺.

### Compound A134. 5-(4-((R)-1-((2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-2-(tetrahydro-2H-pyran-4-yl) acetyl)-4-hydroxypyrrolidine-2-carboxamido)-2-morpholinoethyl)phenyl)thiazole-4-carboxylic acid

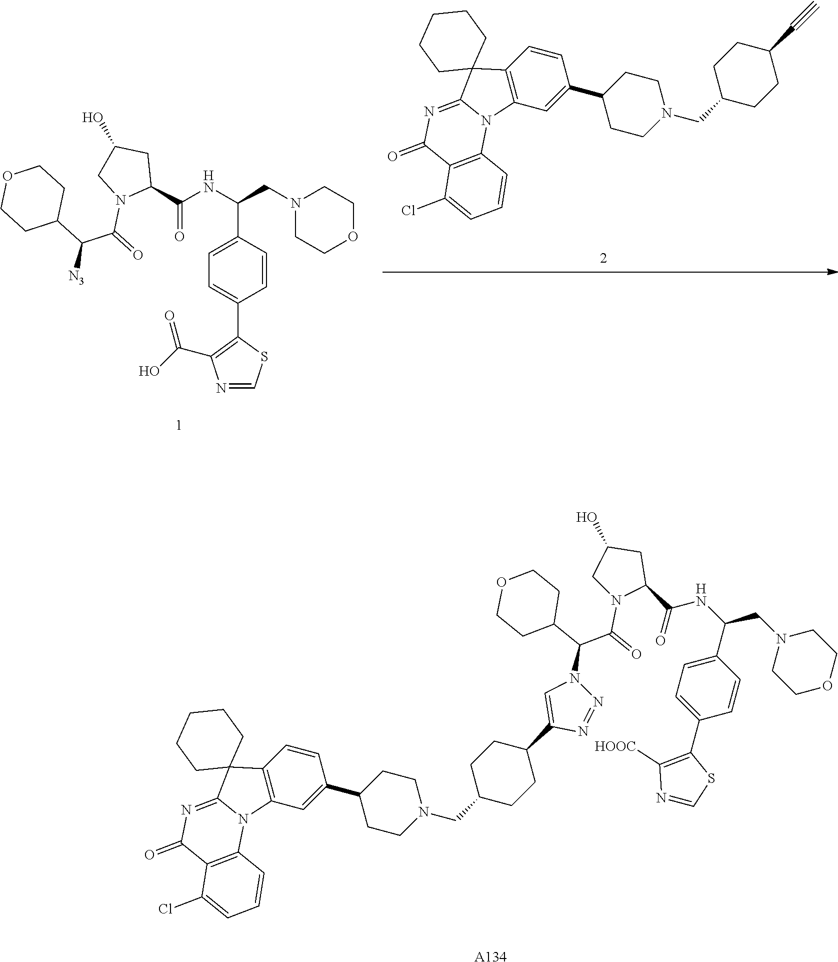

A solution of 1 (30 mg, 0.048 mmol, 1.0 eq), 2 (26 mg, 0.048 mmol, 1.0 eq), sodium ascorbate (20 mg, 0.1 mmol, 2.0 eq), CuSO₄·10 (H₂O) (12.5 mg, 0.05 mmol, 1.0 eq) in t-BuOH/H₂O (20/10 mL) was stirred overnight. The reaction solution was concentrated. The residue was purified by pre-HPLC (elution solvents: CH₃CN/H₂O from 10% to 100%, 0.1% TFA) to get target product 3 (30 mg, 53%) as a slight yellow solid. ESI MS m/z: 1153.50 [M+H]⁺.

### Compound A135. (2S,4R)—N—((R)-2-(4-(4′-bromo-5′-oxo-5′H-spiro[cyclohexane-1,7′-indolo[1,2-a]quinazolin]-10′-yl)piperidin-1-yl)-1-(4-(4-methylthiazol-5-yl)phenyl)-2-oxoethyl)-1-((S)-2-(4-cyclopropyl-1H-1,2,3-triazol-1-yl)-3,3-dimethylbutanoyl)-4-hydroxypyrrolidine-2-carboxamide

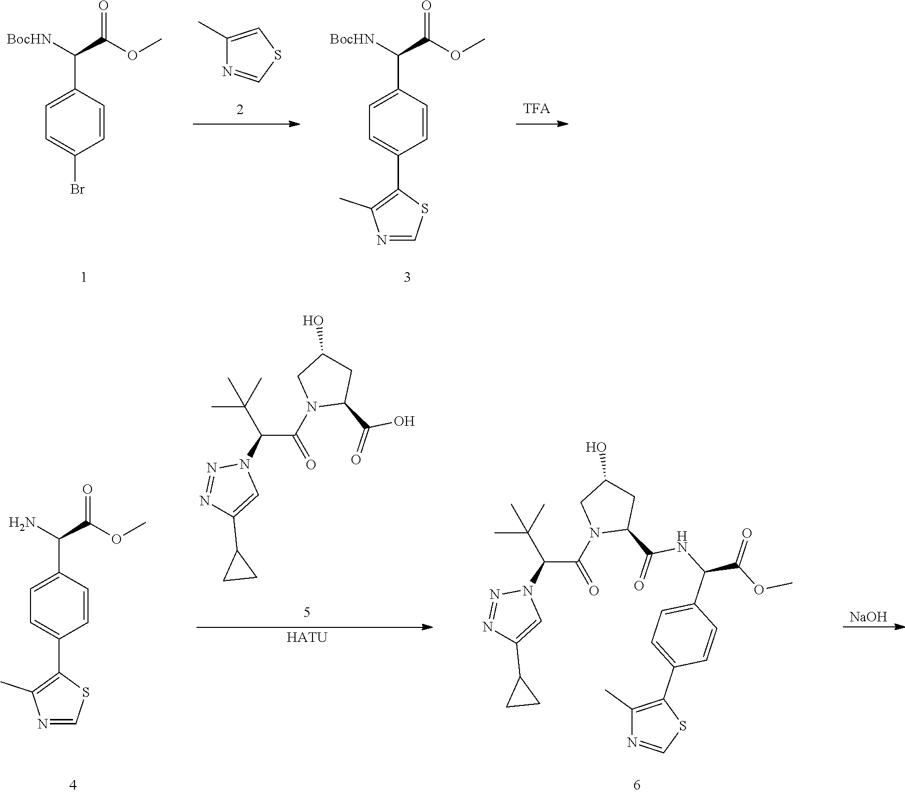

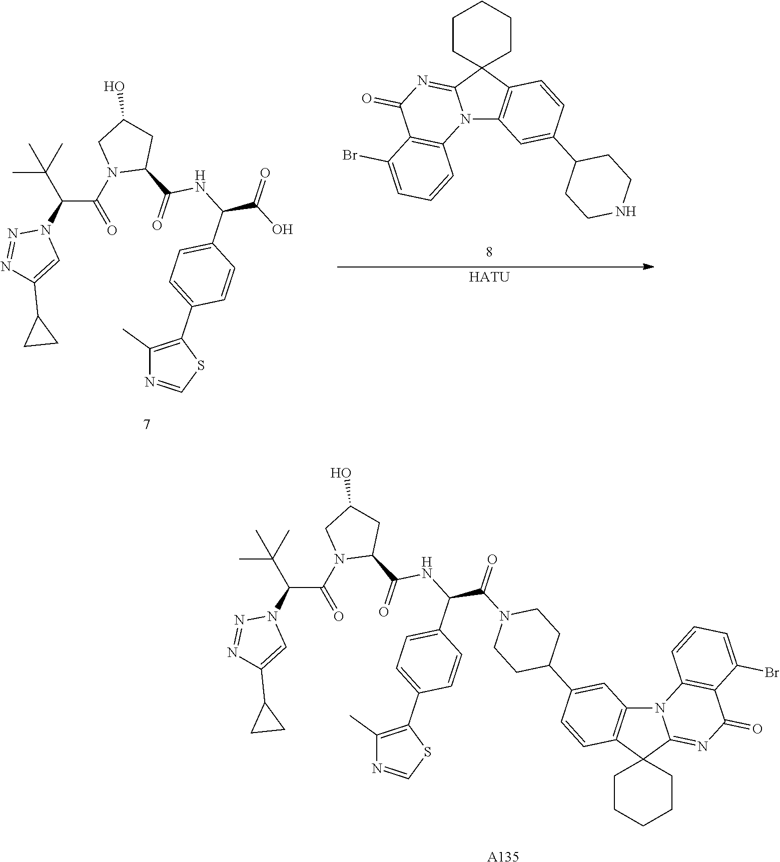 [SMILES: Cc1ncsc1-c1ccc([C@@H](NC(=O)[C@@H]2C[C@@H](O)CN2C(=O)[C@@H](n2cc(C3CC3)nn2)C(C)(C)C)C(=O)O)cc1 ; O=c1nc2n(c3cccc(Br)c13)-c1cc(C3CCNCC3)ccc1C21CCCCC1 ; "HATU" ; Cc1ncsc1-c1ccc([C@@H](NC(=O)[C@@H]2C[C@@H](O)CN2C(=O)[C@@H](n2cc(C3CC3)nn2)C(C)(C)C)C(=O)N2CCC(c3ccc4c(c3)-n3c(nc(=O)c5c(Br)cccc53)C43CCCCC3)CC2)cc1 ; "A135"]

### Synthesis of methyl(R)-2-((tert-butoxycarbonyl)amino)-2-(4-(4-methylthiazol-5-yl)phenyl)acetate. 3

To a solution of 1 (3.00, 8.72 mmol, 1.0 eq) in DMA (50 mL), was added 2 (2.60 g, 26.15 mmol, 3.0 eq), K₂CO₃ (2.41 g, 17.43 mmol, 2.0 eq), Pd (OAc) 2 (195 mg, 0.87 mmol, 0.1 eq). under nitrogen protection, the mixture was stirred for 6 h at 100° C. After reaction, water was added to the solution, the desired product was extracted with EA twice. Combined organic layers were washed by water twice. After dried by anhydrous Na₂SO₄ and concentrated. The residue was purified by silica gel column chromatography (elution solvents: EA/hexane=1/1, v/v) to get target product 2 (2.6 g, 82%) as a slight yellow solid. ESI MS m/z: 363.13 [M+H]⁺.

### Synthesis of (R)-2-((2S,4R)-1-((S)-2-(4-cyclopropyl-1H-1,2,3-triazol-1-yl)-3,3-dimethylbutanoyl)-4-hydroxypyrrolidine-2-carboxamido)-2-(4-(4-methylthiazol-5-yl)phenyl) acetic acid, 6

To a solution of 4 (2.50 g, 6.90 mmol, 1.0 eq) in DCM (40 mL), TFA (20 mL) was added, the mixture stirred for 30 minutes. After reaction the solution was concentrated. The residue was dissolved into DMF (50 mL). 3 (2.32 g, 6.90 mmol, 1.0 eq), HATU (3.93 g, 10.35 mmol, 1.5 eq), and DIPEA (2.67 g, 20.69 mmol, 3.0 eq). The mixture was stirred for 30 minutes. The reaction was quenched by added water. The mixture was extracted by ethyl acetate (60*2 mL) twice.

Combined organic phases were washed by water twice. After dried by anhydrous Na₂SO₄ and concentrated. The residue was dissolved into MeOH (50 mL), then an aqueous solution of NaOH (1 N, 10 mL), the mixture was stirred for 30 min. After reaction, HCl was added to neutralize the mixture until pH reach 2-3. The reaction was concentrated and purified by C-18 reversal column chromatography (elution solvents: CH₃CN/H₂O from 10% to 100%, 0.1% TFA) to get target product 7 (1.8 g, 45%) as a slight yellow solid. ESI MS m/z: 567.23 [M+H]⁺.

### Synthesis of (2S,4R)—N—((R)-2-(4-(4′-bromo-5′-oxo-5′H-spiro[cyclohexane-1,7′-indolo[1,2-a]quinazolin]-10′-yl)piperidin-1-yl)-1-(4-(4-methylthiazol-5-yl)phenyl)-2-oxoethyl)-1-((S)-2-(4-cyclopropyl-1H-1,2,3-triazol-1-yl)-3,3-dimethylbutanoyl)-4-hydroxypyrrolidine-2-carboxamide, 9

To a solution of 7 (50 mg, 0.088 mmol, 1.0 eq) in DMF (5 mL) was 8 (41 mg, 0.088 mmol, 1.0 eq), HATU (50 mg, 0.13 mmol, 1.5 eq), and DIPEA (34 mg, 0.26 mmol, 3.0 eq). The mixture was stirred for 30 minutes. The residue was purified by pre-HPLC (elution solvents: CH₃CN/H₂O from 10% to 100%, 0.1% TFA) to get target product A¹³⁵ (42 mg, 47%) as a slight yellow solid. ESI MS m/z: 1014.34 [M+H]⁺.

### Compound A136. (2S,4R)—N—((R)-2-(4-((4-(4′-bromo-5′-oxo-5′H-spiro[cyclohexane-1,7′-indolo[1,2-a]quinazolin]-10′-yl)piperidin-1-yl)methyl)piperidin-1-yl)-1-(4-(4-methylthiazol-5-yl)phenyl)-2-oxoethyl)-1-((S)-2-(4-cyclopropyl-1H-1,2,3-triazol-1-yl)-3,3-dimethylbutanoyl)-4-hydroxypyrrolidine-2-carboxamide

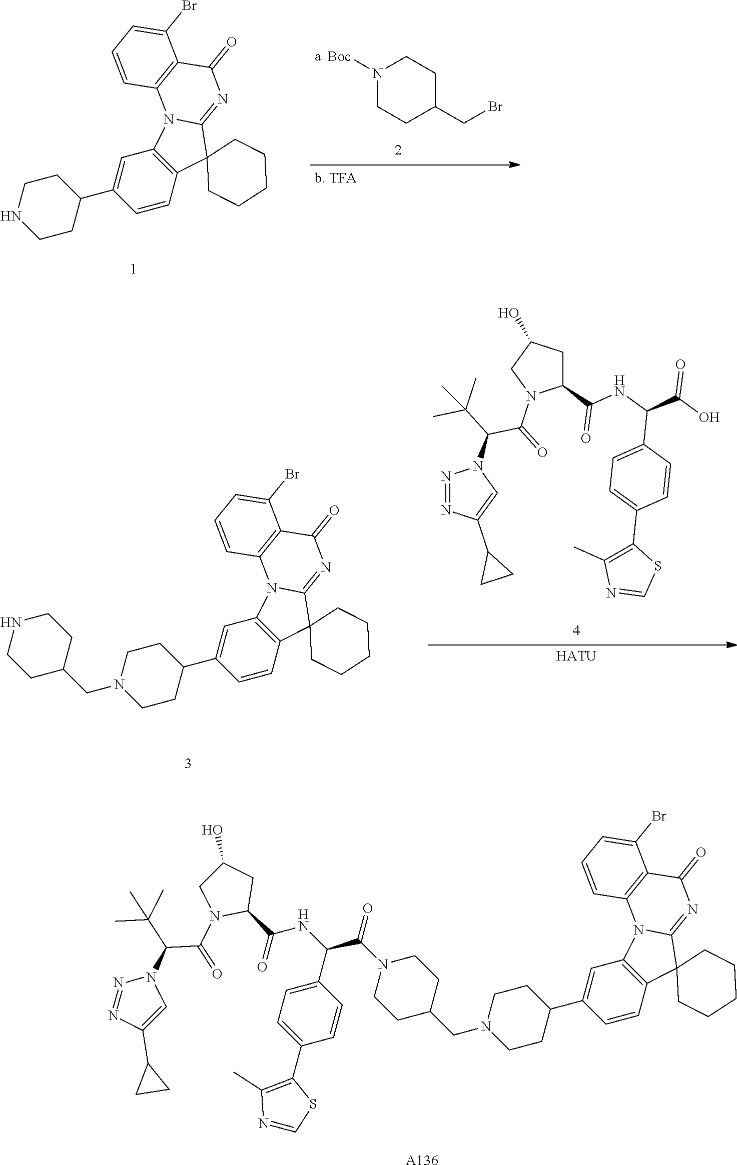

### Synthesis of 4′-bromo-10′-(1-(piperidin-4-ylmethyl)piperidin-4-yl)-5′H-spiro[cyclohexane-1,7′-indolo[1,2-a]quinazolin]-5′-one, 3

To a solution of 1 (100 mg, 0.22 mmol, 1.0 eq) in DMF (5 mL), was added 2 (119 mg, 0.43 mmol, 2.0 eq), and DIPEA (135 mg, 0.86 mmol, 3.0 eq). The mixture was stirred for 16 h at 100° C. The reaction was quenched by added water. The mixture was extracted by ethyl acetate (20*2 mL) twice. Combined organic phases were washed by water twice. After dried by anhydrous Na₂SO₄ and concentrated. The residue was dissolved into DCM (5 mL), TFA was added, the mixture was stirred for 30 min. After reaction, the reside was purified by C-18 reversal column chromatography (elution solvents: CH₃CN/H₂O from 10% to 100%, 0.1% TFA) to get target product 3 (80 mg, 66%) as a slight yellow solid. ESI MS m/z: 561.22 [M+H]⁺.

### Synthesis of (2S,4R)—N—((R)-2-(4-((4-(4′-bromo-5′-oxo-5′H-spiro[cyclohexane-1,7′-indolo[1,2-a]quinazolin]-10′-yl)piperidin-1-yl)methyl)piperidin-1-yl)-1-(4-(4-methylthiazol-5-yl)phenyl)-2-oxoethyl)-1-((S)-2-(4-cyclopropyl-1H-1,2,3-triazol-1-yl)-3,3-dimethylbutanoyl)-4-hydroxypyrrolidine-2-carboxamide, 5

To a solution of 3 (50 mg, 0.088 mmol, 1.0 eq) in DMF (5 mL) was 4 (50 mg, 0.088 mmol, 1.0 eq), HATU (50 mg, 0.13 mmol, 1.5 eq), and DIPEA (34 mg, 0.26 mmol, 3.0 eq). The mixture was stirred for 30 minutes. The residue was purified by pre-HPLC (elution solvents: CH₃CN/H₂O from 10% to 100%, 0.1% TFA) to get target product A¹³⁶ (62 mg, 63%) as a slight yellow solid. ESI MS m/z: 1111.43 [M+H]⁺.

### Compound A137. (2S,4R)—N—((R)-2-(9-((4-(4′-bromo-5′-oxo-5′H-spiro[cyclohexane-1,7′-indolo[1,2-a]quinazolin]-10′-yl)piperidin-1-yl)methyl)-3-azaspiro[5.5]undecan-3-yl)-1-(4-(4-methylthiazol-5-yl)phenyl)-2-oxoethyl)-1-((S)-2-(4-cyclopropyl-1H-1,2,3-triazol-1-yl)-3,3-dimethylbutanoyl)-4-hydroxypyrrolidine-2-carboxamide

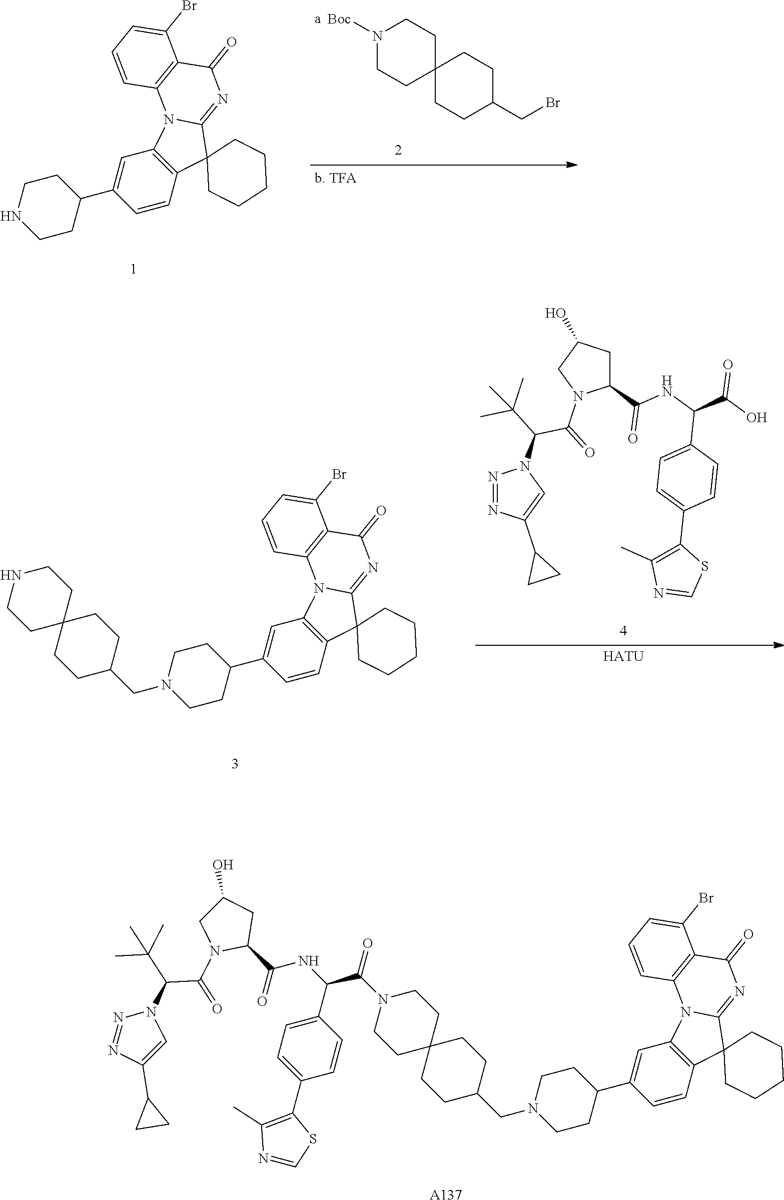

As showed above figure. The procedure is similar as former procedure of preparing compound A136, except changing the recording different reagents. The target degrader was obtained as while solid. ESI MS m/z: 1179.50 [M+H]⁺.

### Compound A138. (2S,4R)—N—((R)-2-(4-(4′-bromo-5′-oxo-5′H-spiro[cyclohexane-1,7′-indolo[1,2-a]quinazolin]-10′-yl)-[1,4′-bipiperidin]-1′-yl)-1-(4-(4-methylthiazol-5-yl)phenyl)-2-oxoethyl)-1-((S)-2-(4-cyclopropyl-1H-1,2,3-triazol-1-yl)-3,3-dimethylbutanoyl)-4-hydroxypyrrolidine-2-carboxamide

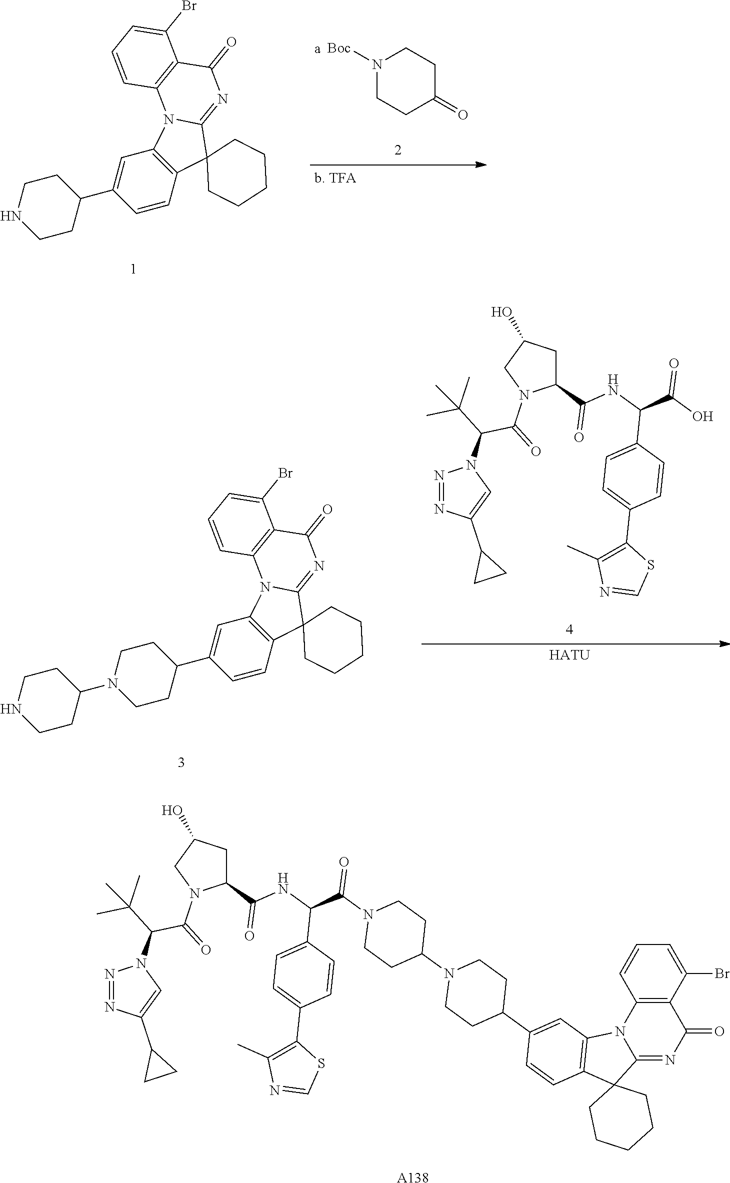

As showed above figure. The first step is similar as the former reductive amination. The procedure is similar as the former of preparing compound A136 except changing the recording different reagents. The target degrader was obtained as while solid. ESI MS m/z: 1097.42 [M+H]⁺.

### Compound A139. (2S,4R)—N—((R)-2-(9-(4-(4′-bromo-5′-oxo-5′H-spiro[cyclohexane-1,7′-indolo[1,2-a]quinazolin]-10′-yl)piperidin-1-yl)-3-azaspiro[5.5]undecan-3-yl)-1-(4-(4-methylthiazol-5-yl)phenyl)-2-oxoethyl)-1-((S)-2-(4-cyclopropyl-1H-1,2,3-triazol-1-yl)-3,3-dimethylbutanoyl)-4-hydroxypyrrolidine-2-carboxamide

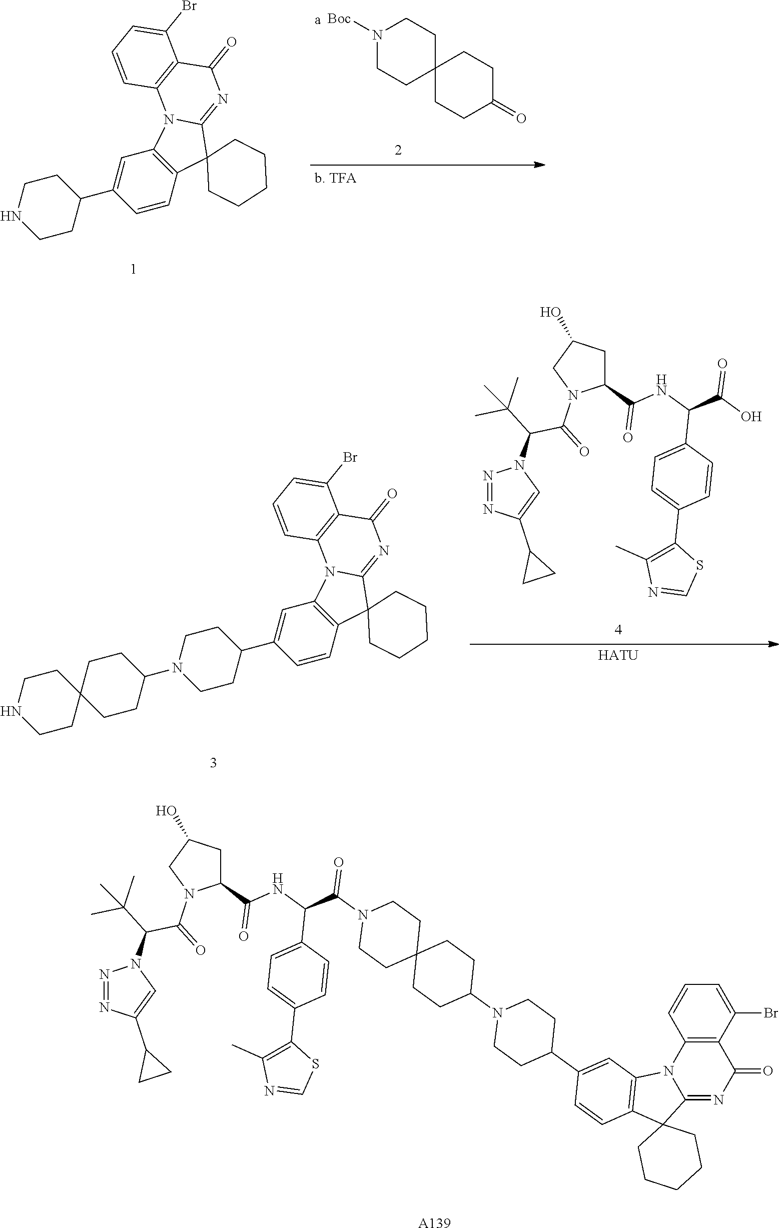

As showed above figure. The first step is similar as the former reductive amination. The procedure is similar to the former of preparing compound A136, except changing the recording different reagents. The target degrader was obtained as while solid. ESI MS m/z: 1165.48 [M+H]⁺.

### Compound A140. (2S,4R)—N—((S)-3-(4-(4′-chloro-5′-oxo-5′H-spiro[cyclohexane-1,7′-indolo[1,2-a]quinazolin]-10′-yl)piperidin-1-yl)-1-(4-(4-methylthiazol-5-yl)phenyl)-3-oxopropyl)-1-((S)-2-(4-cyclopropyl-1H-1,2,3-triazol-1-yl)-3,3-dimethylbutanoyl)-4-hydroxypyrrolidine-2-carboxamide

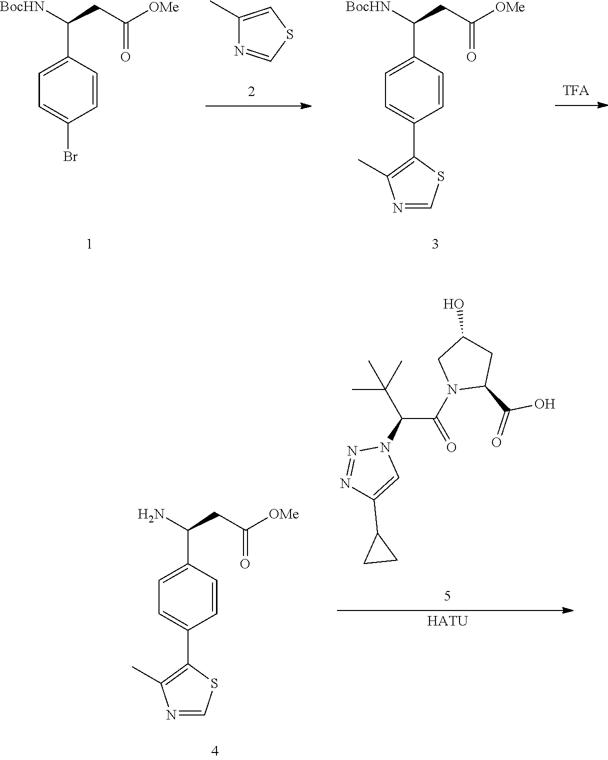

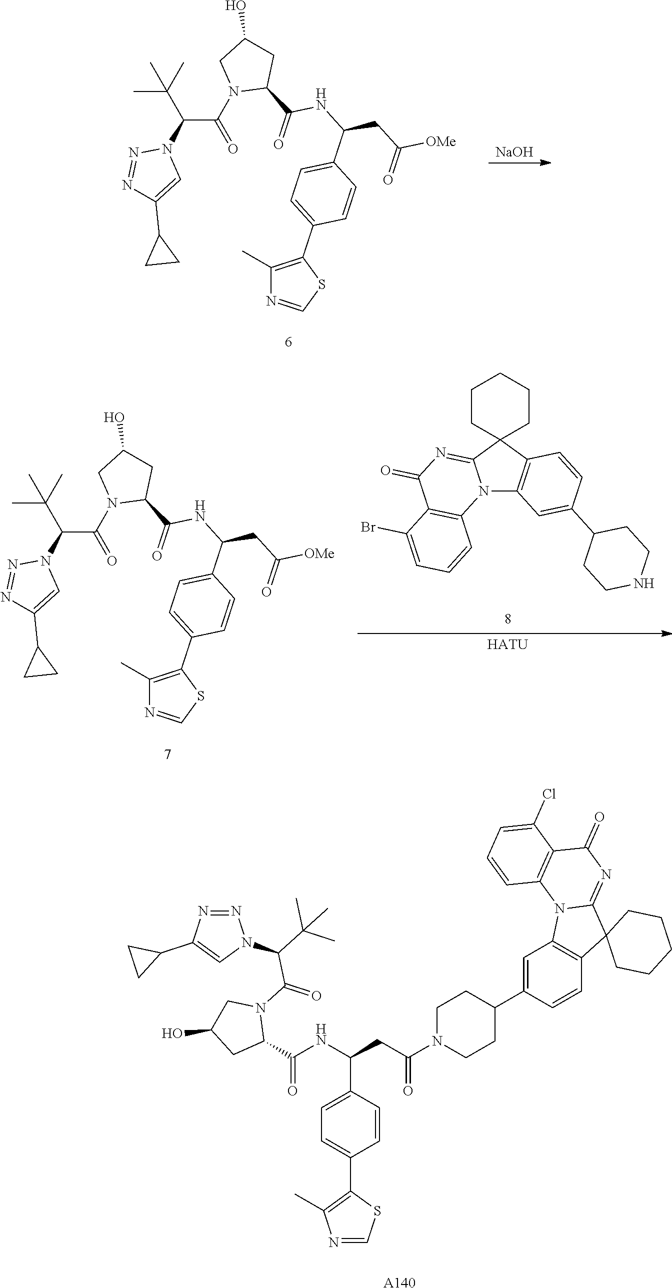

As showed above figure. The procedure is similar as former of preparing A¹³⁵, except changing the recording different reagents. The target degrader was obtained as while solid. ESI MS m/z: 982.41 [M+H]⁺.

### Compound A141. (2S,4R)—N—((S)-3-(4-((4-(4′-bromo-5′-oxo-5′H-spiro[cyclohexane-1,7′-indolo[1,2-a]quinazolin]-10′-yl)piperidin-1-yl)methyl)piperidin-1-yl)-1-(4-(4-methylthiazol-5-yl)phenyl)-3-oxopropyl)-1-((S)-2-(4-cyclopropyl-1H-1,2,3-triazol-1-yl)-3,3-dimethylbutanoyl)-4-hydroxypyrrolidine-2-carboxamide

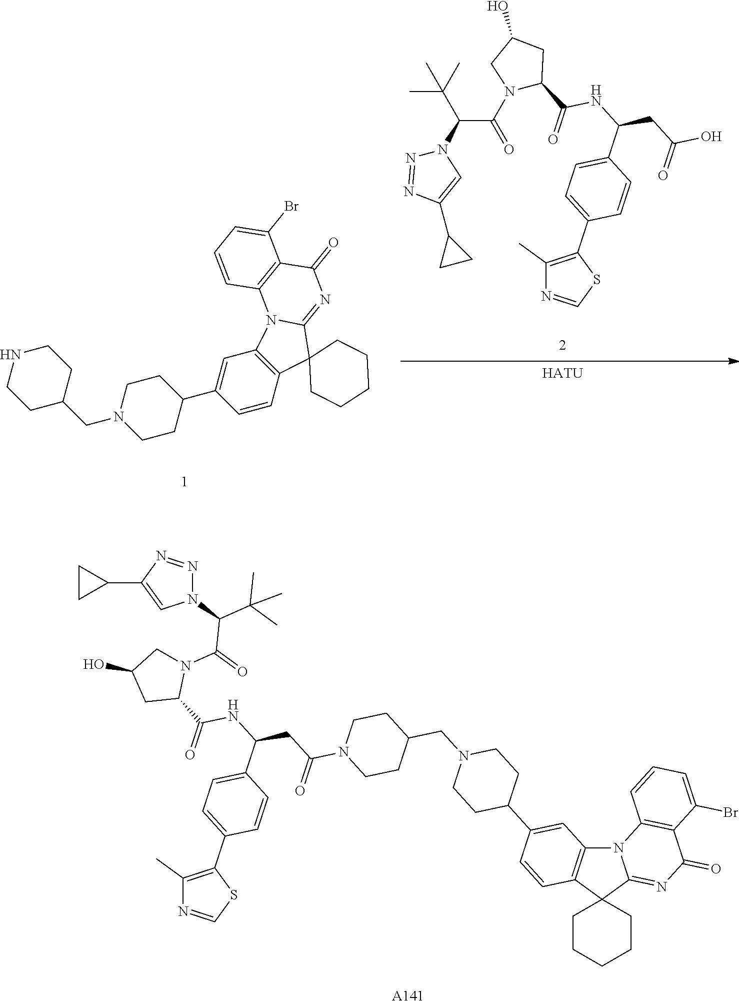 [SMILES: O=c1nc2n(c3cccc(Br)c13)-c1cc(C3CCN(CC4CCNCC4)CC3)ccc1C21CCCCC1 ; Cc1ncsc1-c1ccc([C@H](CC(=O)O)NC(=O)[C@@H]2C[C@@H](O)CN2C(=O)[C@@H](n2cc(C3CC3)nn2)C(C)(C)C)cc1 ; "HATU" ; Cc1ncsc1-c1ccc([C@H](CC(=O)N2CCC(CN3CCC(c4ccc5c(c4)-n4c(nc(=O)c6c(Br)cccc64)C54CCCCC4)CC3)CC2)NC(=O)[C@@H]2C[C@@H](O)CN2C(=O)[C@@H](n2cc(C3CC3)nn2)C(C)(C)C)cc1 ; "A141"]

As showed above figure. The procedure is similar to the former of preparing A¹³⁶ except changing the recording different reagents. The target degrader was obtained as while solid. ESI MS m/z: 1125.45 [M+H]⁺.

### Compound A142. (2S,4R)—N—((S)-3-(9-((4-(4′-bromo-5′-oxo-5′H-spiro[cyclohexane-1,7′-indolo[1,2-a]quinazolin]-10′-yl)piperidin-1-yl)methyl)-3-azaspiro[5.5]undecan-3-yl)-1-(4-(4-methylthiazol-5-yl)phenyl)-3-oxopropyl)-1-((S)-2-(4-cyclopropyl-1H-1,2,3-triazol-1-yl)-3,3-dimethylbutanoyl)-4-hydroxypyrrolidine-2-carboxamide

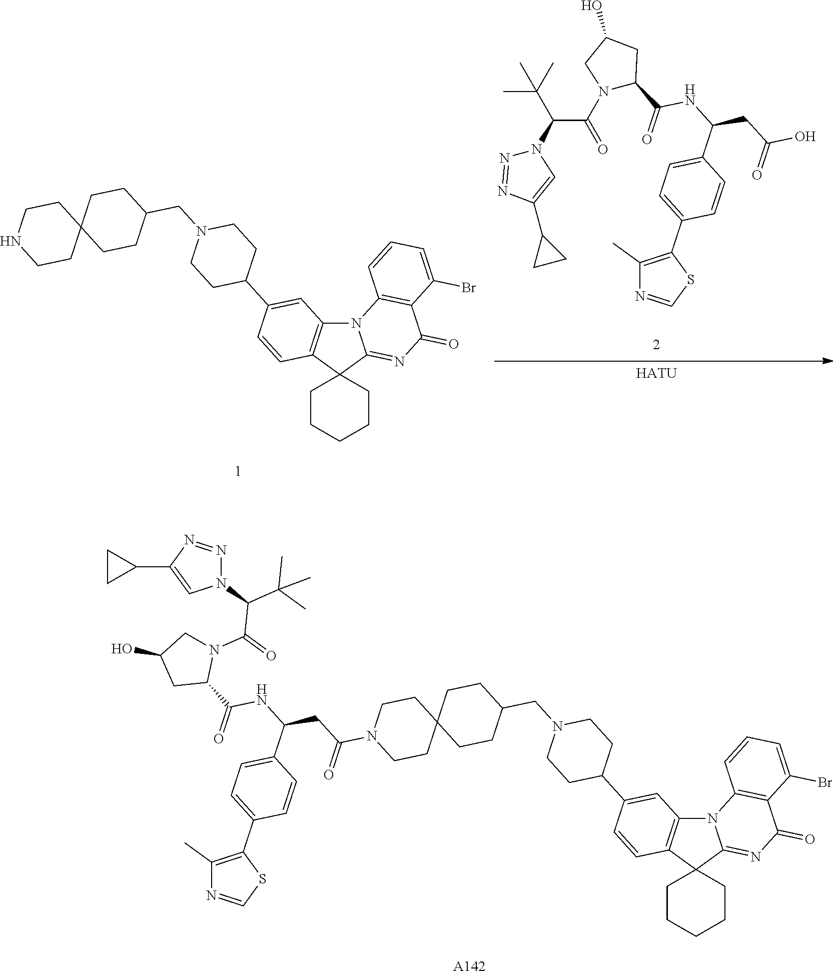 [SMILES: O=c1nc2n(c3cccc(Br)c13)-c1cc(C3CCN(CC4CCC5(CCNCC5)CC4)CC3)ccc1C21CCCCC1 ; Cc1ncsc1-c1ccc([C@H](CC(=O)O)NC(=O)[C@@H]2C[C@@H](O)CN2C(=O)[C@@H](n2cc(C3CC3)nn2)C(C)(C)C)cc1 ; "HATU" ; "1" ; Cc1ncsc1-c1ccc([C@H](CC(=O)N2CCC3(CCC(CN4CCC(c5ccc6c(c5)-n5c(nc(=O)c7c(Br)cccc75)C65CCCCC5)CC4)CC3)CC2)NC(=O)[C@@H]2C[C@@H](O)CN2C(=O)[C@@H](n2cc(C3CC3)nn2)C(C)(C)C)cc1 ; "A142"]

As showed above figure. The procedure is similar as the former of preparing A¹³⁶ except changing the recording different reagents. The target degrader was obtained as while solid. ESI MS m/z: 1193.50 [M+H]⁺.

### Compound A143. (2S,4R)—N—((S)-3-(4-(4′-chloro-5′-oxo-5′H-spiro[cyclohexane-1,7′-indolo[1,2-a]quinazolin]-10′-yl)-[1,4′-bipiperidin]-1′-yl)-1-(4-(4-methylthiazol-5-yl)phenyl)-3-oxopropyl)-1-((S)-2-(4-cyclopropyl-1H-1,2,3-triazol-1-yl)-3,3-dimethylbutanoyl)-4-hydroxypyrrolidine-2-carboxamide

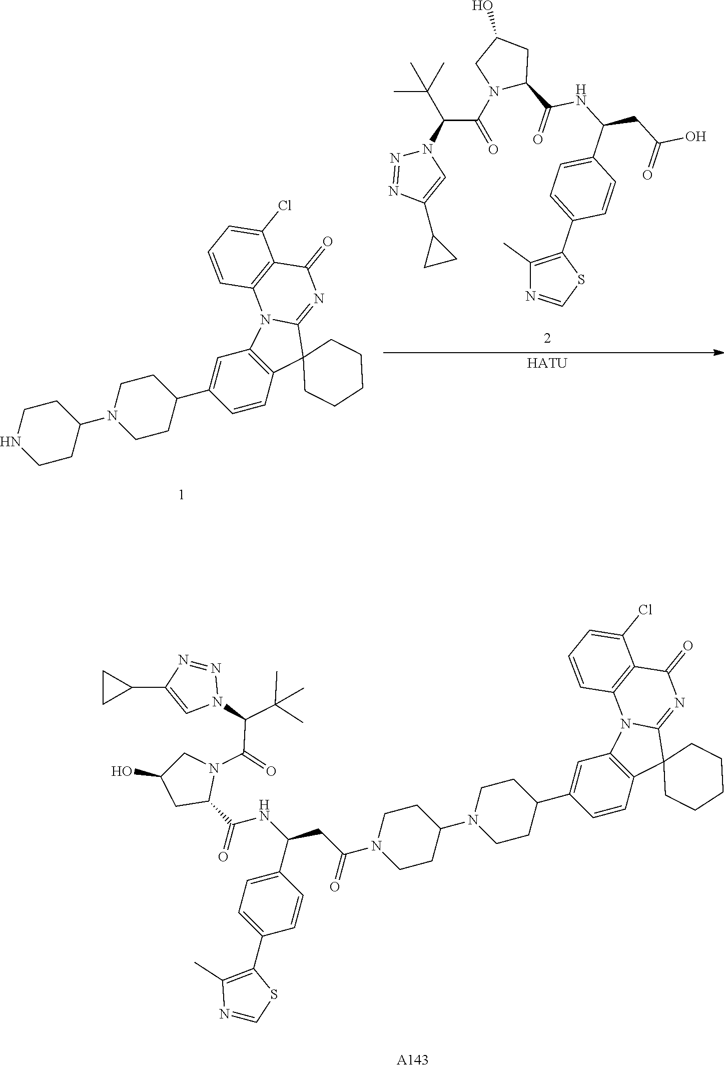 [SMILES: O=c1nc2n(c3cccc(Cl)c13)-c1cc(C3CCN(C4CCNCC4)CC3)ccc1C21CCCCC1 ; Cc1ncsc1-c1ccc([C@H](CC(=O)O)NC(=O)[C@@H]2C[C@@H](O)CN2C(=O)[C@@H](n2cc(C3CC3)nn2)C(C)(C)C)cc1 ; "HATU" ; Cc1ncsc1-c1ccc([C@H](CC(=O)N2CCC(N3CCC(c4ccc5c(c4)-n4c(nc(=O)c6c(Cl)cccc64)C54CCCCC4)CC3)CC2)NC(=O)[C@@H]2C[C@@H](O)CN2C(=O)[C@@H](n2cc(C3CC3)nn2)C(C)(C)C)cc1 ; "A143"]

As showed above figure. The first step is similar as the former reductive amination. The procedure is similar as the former of preparing A¹³⁶ except changing the recording different reagents. The target degrader was obtained as while solid. ESI MS m/z: 1065.49 [M+H]⁺.

### Compound A144. (2S,4R)—N—((S)-3-(9-(4-(4′-bromo-5′-oxo-5′H-spiro[cyclohexane-1,7′-indolo[1,2-a]quinazolin]-10′-yl)piperidin-1-yl)-3-azaspiro[5.5]undecan-3-yl)-1-(4-(4-methylthiazol-5-yl)phenyl)-3-oxopropyl)-1-((S)-2-(4-cyclopropyl-1H-1,2,3-triazol-1-yl)-3,3-dimethylbutanoyl)-4-hydroxypyrrolidine-2-carboxamide

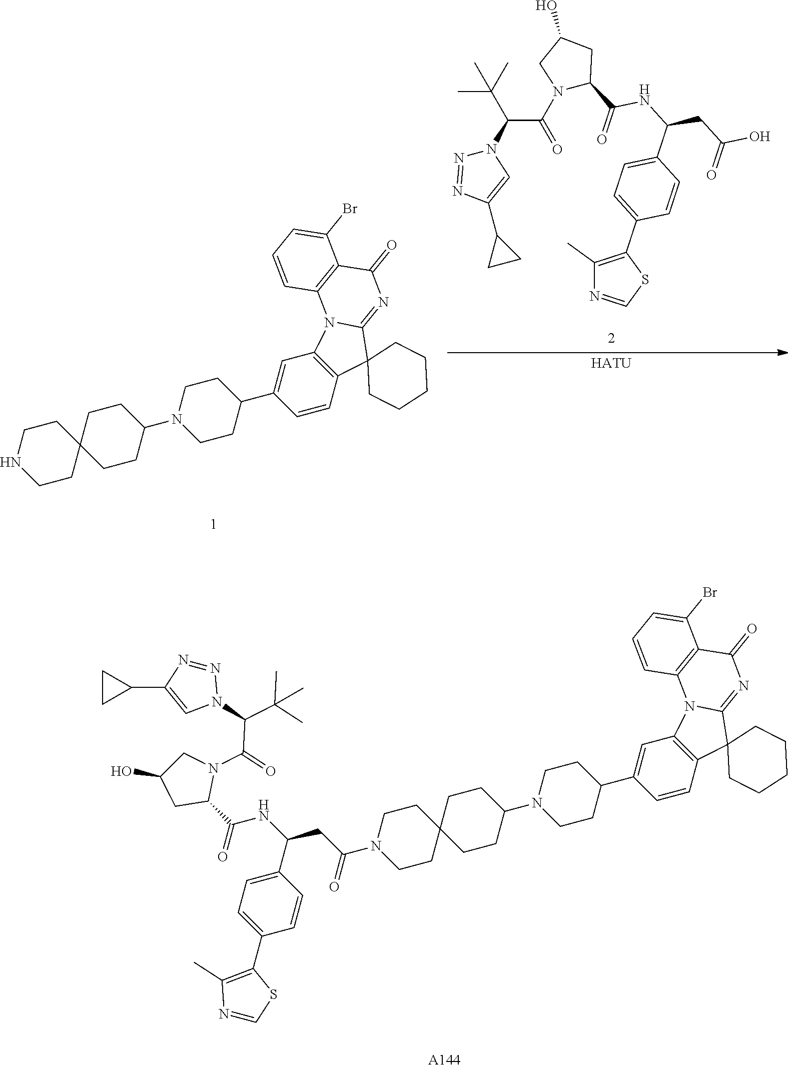 [SMILES: O=c1nc2n(c3cccc(Br)c13)-c1cc(C3CCN(C4CCC5(CCNCC5)CC4)CC3)ccc1C21CCCCC1 ; Cc1ncsc1-c1ccc([C@H](CC(=O)O)NC(=O)[C@@H]2C[C@@H](O)CN2C(=O)[C@@H](n2cc(C3CC3)nn2)C(C)(C)C)cc1 ; "HATU" ; Cc1ncsc1-c1ccc([C@H](CC(=O)N2CCC3(CCC(N4CCC(c5ccc6c(c5)-n5c(nc(=O)c7c(Br)cccc75)C65CCCCC5)CC4)CC3)CC2)NC(=O)[C@@H]2C[C@@H](O)CN2C(=O)[C@@H](n2cc(C3CC3)nn2)C(C)(C)C)cc1 ; "A144"]

As showed above figure. The first step is similar as the former reductive amination. The procedure is similar as the former of preparing A¹³⁶ except changing the recording different reagents. The target degrader was obtained as while solid. ESI MS m/z: 1179.50 [M+H]⁺.

### Compound A145. methyl(S)-3-((2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxypyrrolidine-2-carboxamido)-3-(4-(4-methylthiazol-5-yl)phenyl) propanoate

### Compound A146. (S)-3-((2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxypyrrolidine-2-carboxamido)-3-(4-(4-methylthiazol-5-yl)phenyl) propanoic acid

### Compound A147. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-N—((S)-3-(dimethylamino)-1-(4-(4-methylthiazol-5-yl)phenyl)-3-oxopropyl)-4-hydroxypyrrolidine-2-carboxamide

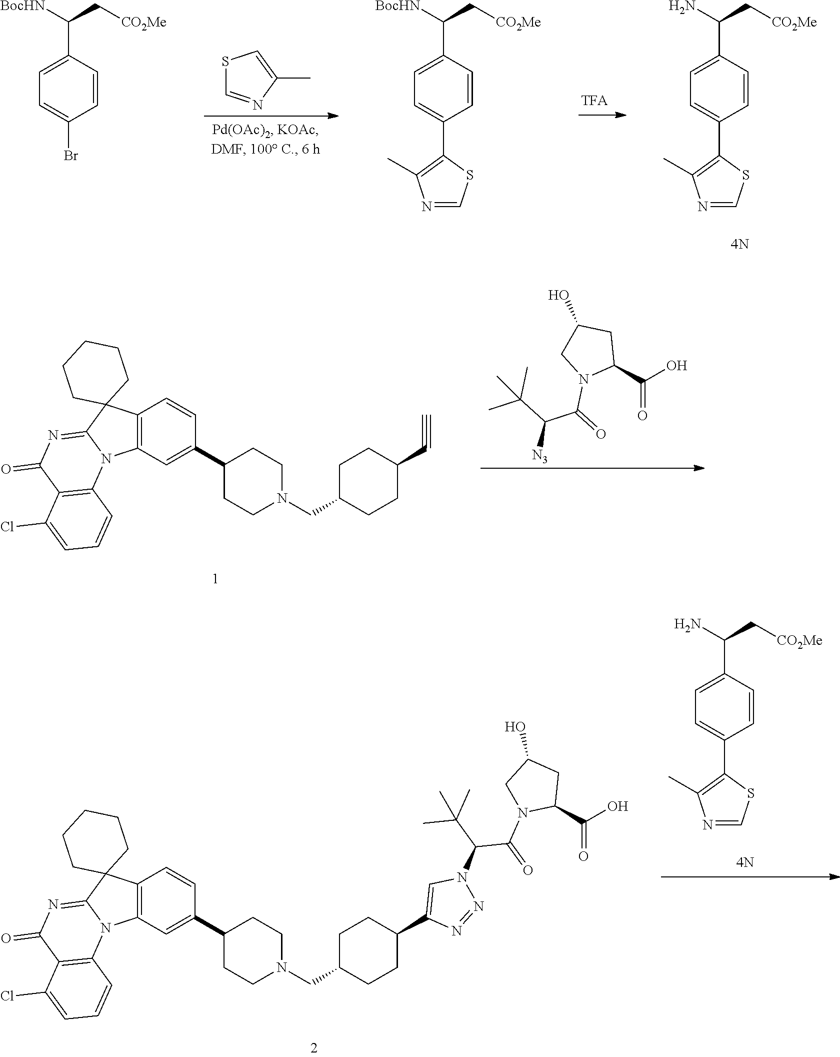

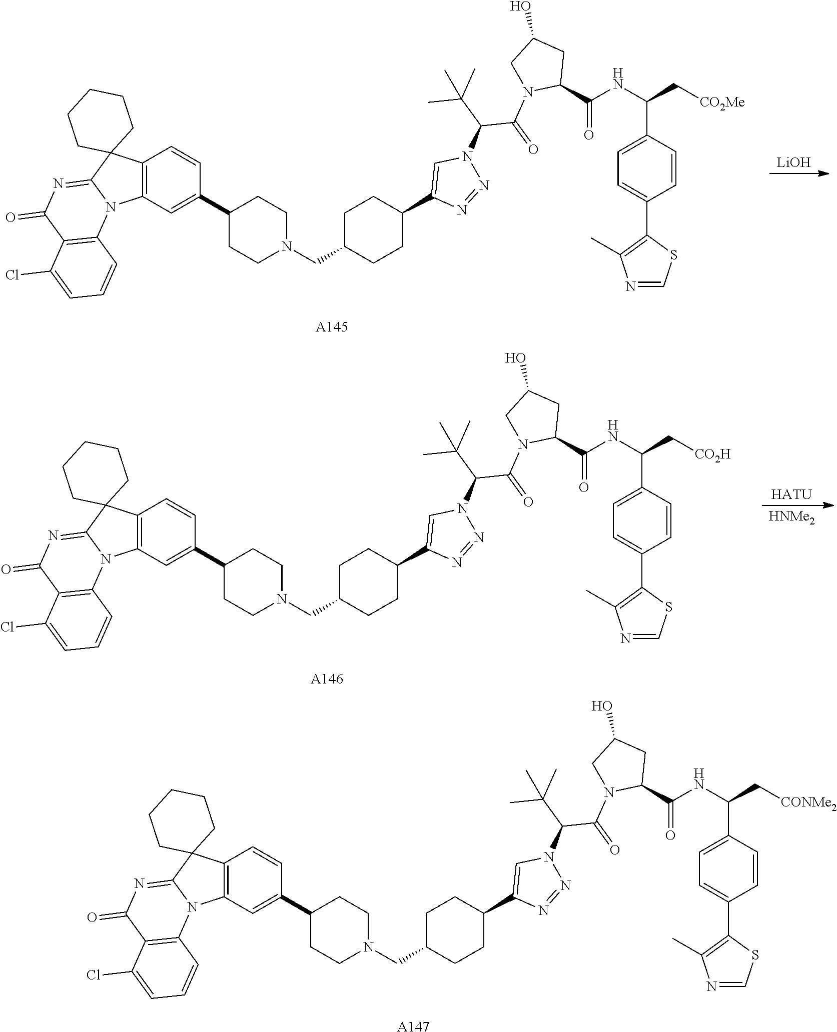

Preparation of 4N: To a solution of methyl(S)-3-(4-bromophenyl)-3-((tert-butoxycarbonyl)amino) propanoate (1.0 g, 2.7 mmol, 1 equiv.) and 4-methylthiazole (0.51 mL, 5.6 mmol, 2.0 equiv.) in DMF (7.3 mL) was added Pd (OAc) 2 (47 mg, 0.27 mmol, 0.1 equiv.) and KOAc (0.55 g, 5.6 mmol, 2 equiv). The mixture was stirred for 6 h at 120° C. The reaction was quenched by water (30 mL) and extracted with ethyl acetate twice (100 mL×2). Combined organic phases were washed by brine twice, dried by anhydrous Na₂SO₄. After removing solvent, the received crude product was purified by silica gel column chromatography (ethyl acetate/hexane=1/1) to get pure product methyl(S)-3-((tert-butoxycarbonyl)amino)-3-(4-(4-methylthiazol-5-yl)phenyl) propanoate (0.57 g, 56%).

To a solution of methyl(S)-3-((tert-butoxycarbonyl)amino)-3-(4-(4-methylthiazol-5-yl)phenyl) propanoate (0.57 g, 1.5 mmol, 1 equiv.) in DCM (10 mL), was added TFA (3 mL) and the mixture was stirred for 30 min. After removing the solvent, the product 4N was received and dried by lyophilizer, and then the crude product can be used in the next step directly.

Compound A145 was synthesized using the similar procedures as used in the synthesis of Compound A67. ESI MS m/z: 1068.48 [M+H]⁺.

Preparation of A¹⁴⁶. To a solution of A¹⁴⁵ (22 mg) in MeOH/THF/H₂O (1/1/1, 4 mL) was added LiOH (22 mg) The mixture was stirred for 3 h. The reaction was quenched by TFA. The mixture was subjected to Prep-HPLC (30-100% MeCN in H₂O, 0.1% TFA) to give compound A¹⁴⁶ (17 mg, 76%). ESI MS m/z: 1054.36 [M+H]⁺.

### Synthesis of Compound 4A, 4B, 4C, 4D, 4E, 4F, 4G, 4H, 4I, 4J, 4K, 4L, and 4M

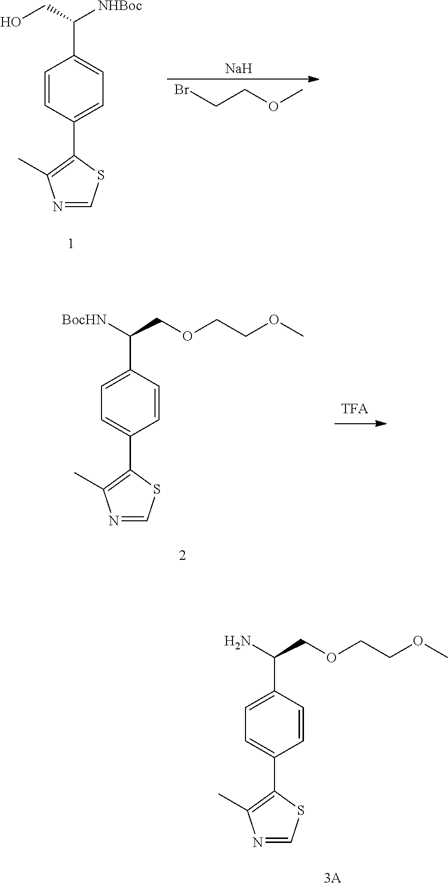

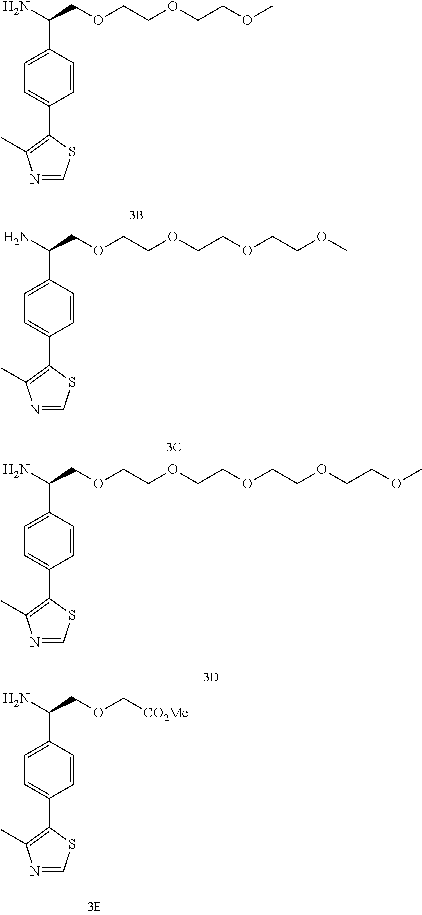

Preparation of 2. To a solution of 1 (100 mg, 0.30 mmol, 1 equiv.) in DMF (3 mL) was added NaH (60% dispersion in mineral oil, 13 mg, 0.33 mmol, 1.1 equiv.) at 0° C. The mixture was stirred at 0° C. for 10 min, 1-bromo-2-methoxyethane (31 μL, 1.1 equiv, 0.33 μmol) was added, and the mixture was stirred at 0° C. for 2 hours. The reaction was quenched by water, and the mixture was subjected to reverse phase flash chromatography (10-100% MeCN in H₂O, 0.1% TFA) to give compound 2 (51 mg, 43%).

Preparation of 3A. To a solution of 2 (51 mg, 0.14 mmol, 1 equiv.) in DCM (3 mL), was added TFA (1 mL) and the mixture was stirred 60 min. After removing solvent, the crude product 4A was received and can be used in the next step directly. ESI MS m/z: 293.33 [M+H]⁺.

Compound 3B-E was synthesized using the similar procedures as used in the synthesis of Compound 3A

### Compound A148. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-13-(4-(4-methylthiazol-5-yl)phenyl)-2,5,8,11-tetraoxatridecan-13-yl)pyrrolidine-2-carboxamide

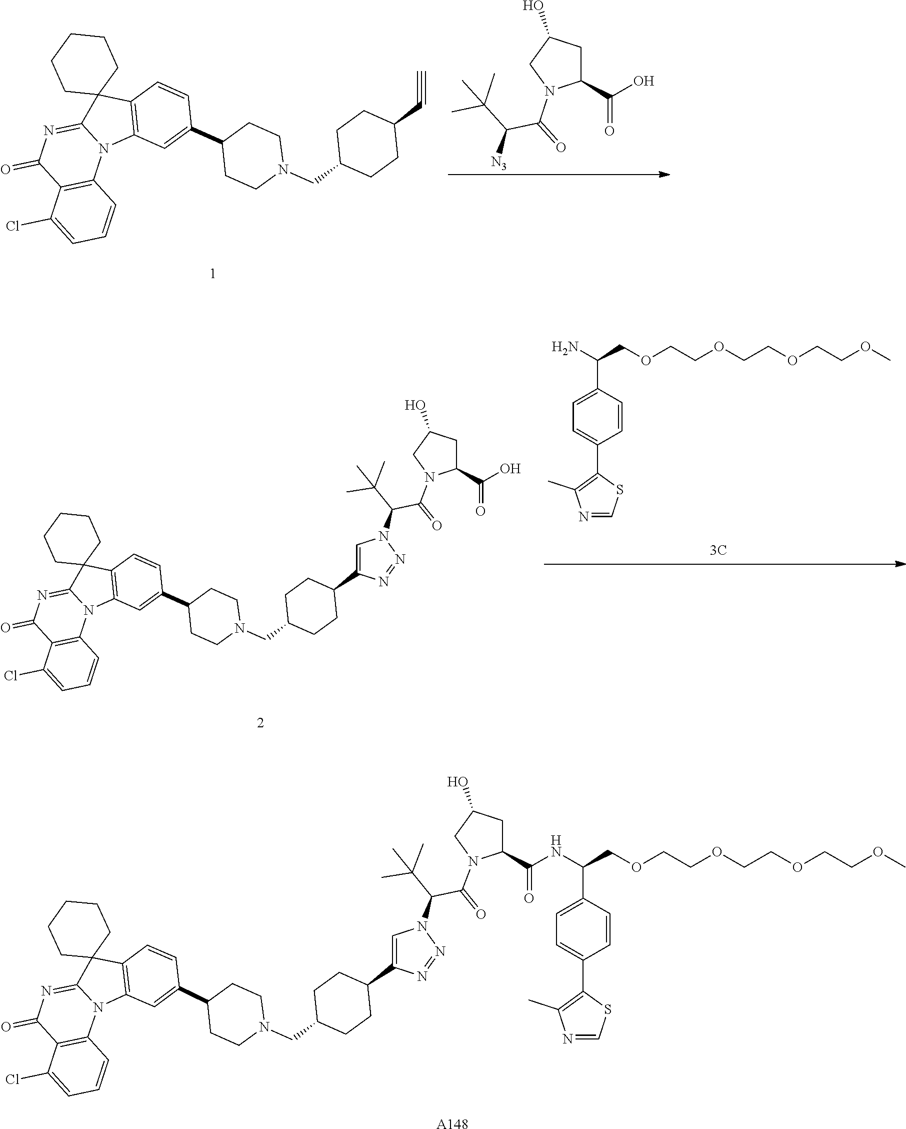

Compound A148 was synthesized using the similar procedures as used in the synthesis of Compound A67. ESI MS m/z: 1172.72 [M+H]⁺.

### Compound A149. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-16-(4-(4-methylthiazol-5-yl)phenyl)-2,5,8,11,14-pentaoxahexadecan-16-yl)pyrrolidine-2-carboxamide

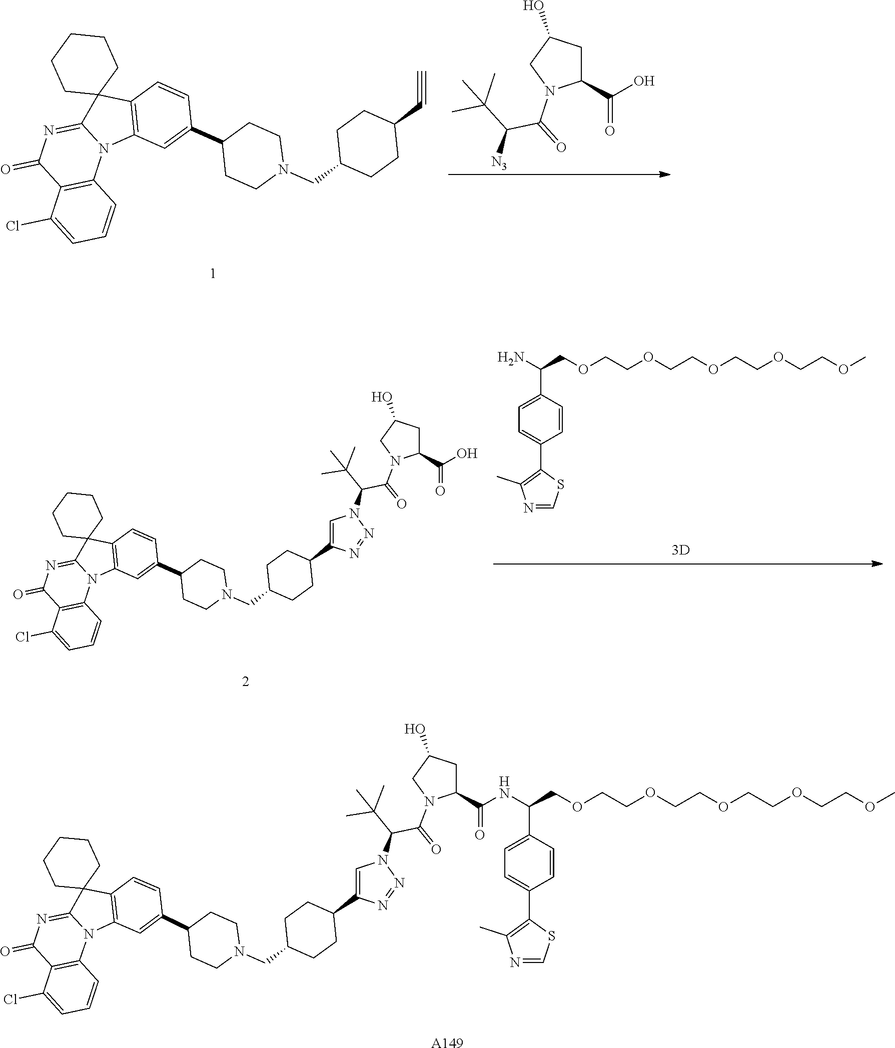

Compound A149 was synthesized using the similar procedures as used in the synthesis of Compound A67. ESI MS m/z: 1216.60 [M+H]⁺.

### Compound A150. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-hydroxy-N—((R)-13-(4-(4-methylthiazol-5-yl)phenyl)-2,5,8,11-tetraoxatridecan-13-yl)pyrrolidine-2-carboxamide

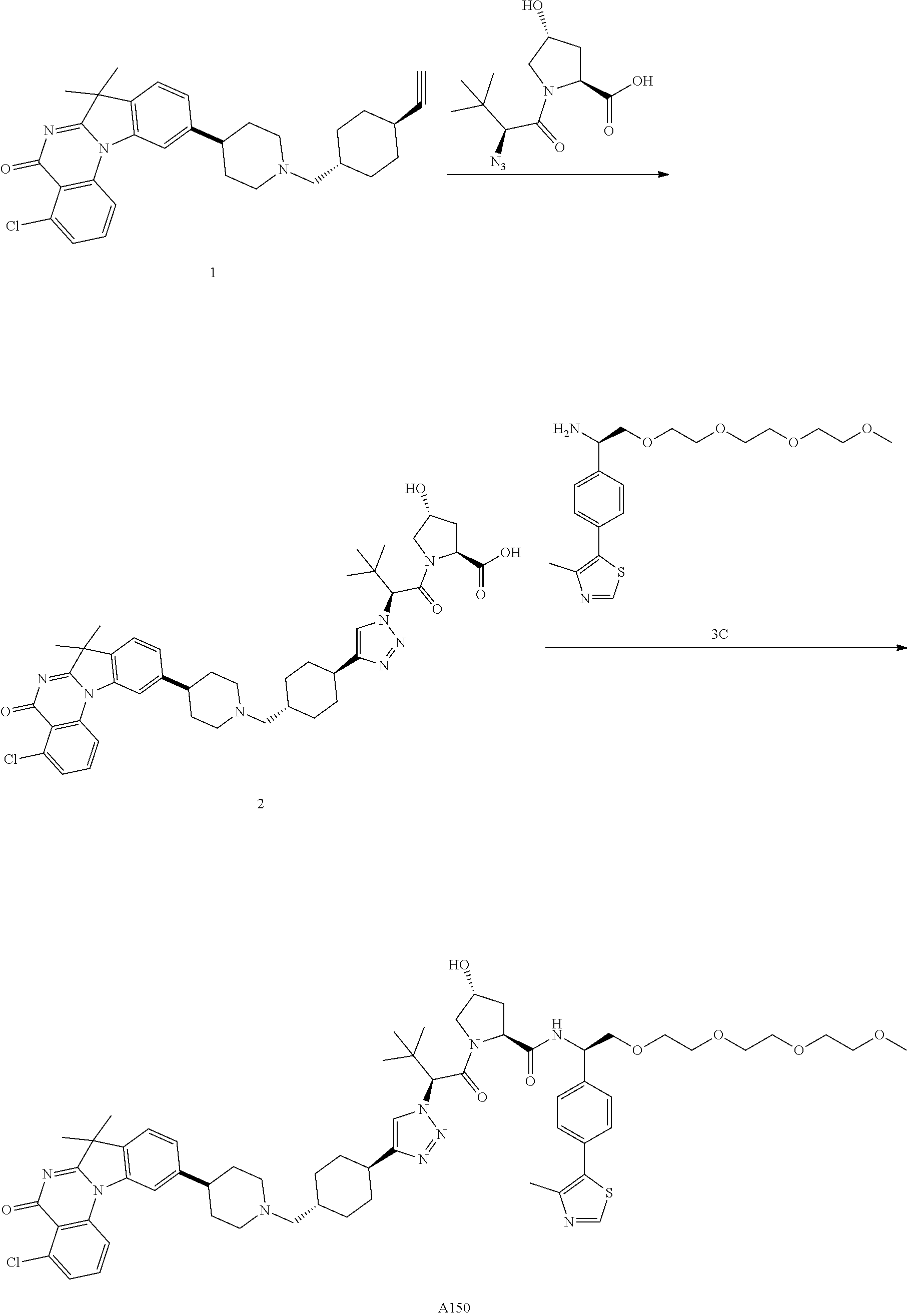

Compound A150 was synthesized using the similar procedures as used in the synthesis of Compound A67. ESI MS m/z: 1132.60 [M+H]⁺.

### Compound A151. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-hydroxy-N—((R)-16-(4-(4-methylthiazol-5-yl)phenyl)-2,5,8,11,14-pentaoxahexadecan-16-yl)pyrrolidine-2-carboxamide

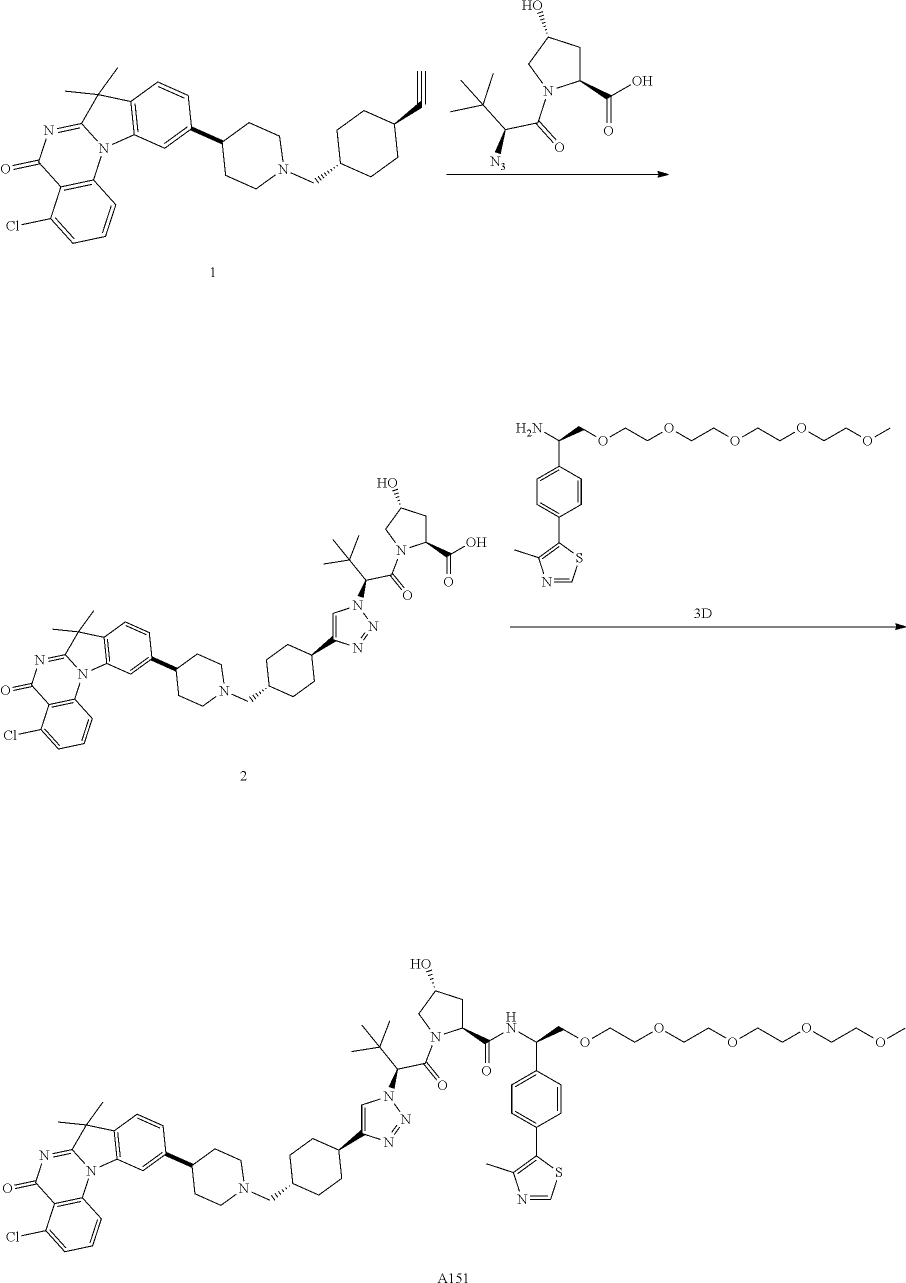

Compound A151 was synthesized using the similar procedures as used in the synthesis of Compound A67. ESI MS m/z: 1176.53 [M+H]⁺.

### Compound A152. methyl 2-((R)-2-((2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxypyrrolidine-2-carboxamido)-2-(4-(4-methylthiazol-5-yl)phenyl)ethoxy)acetate

### Compound A155. 2-((R)-2-((2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxypyrrolidine-2-carboxamido)-2-(4-(4-methylthiazol-5-yl)phenyl)ethoxy) acetic acid

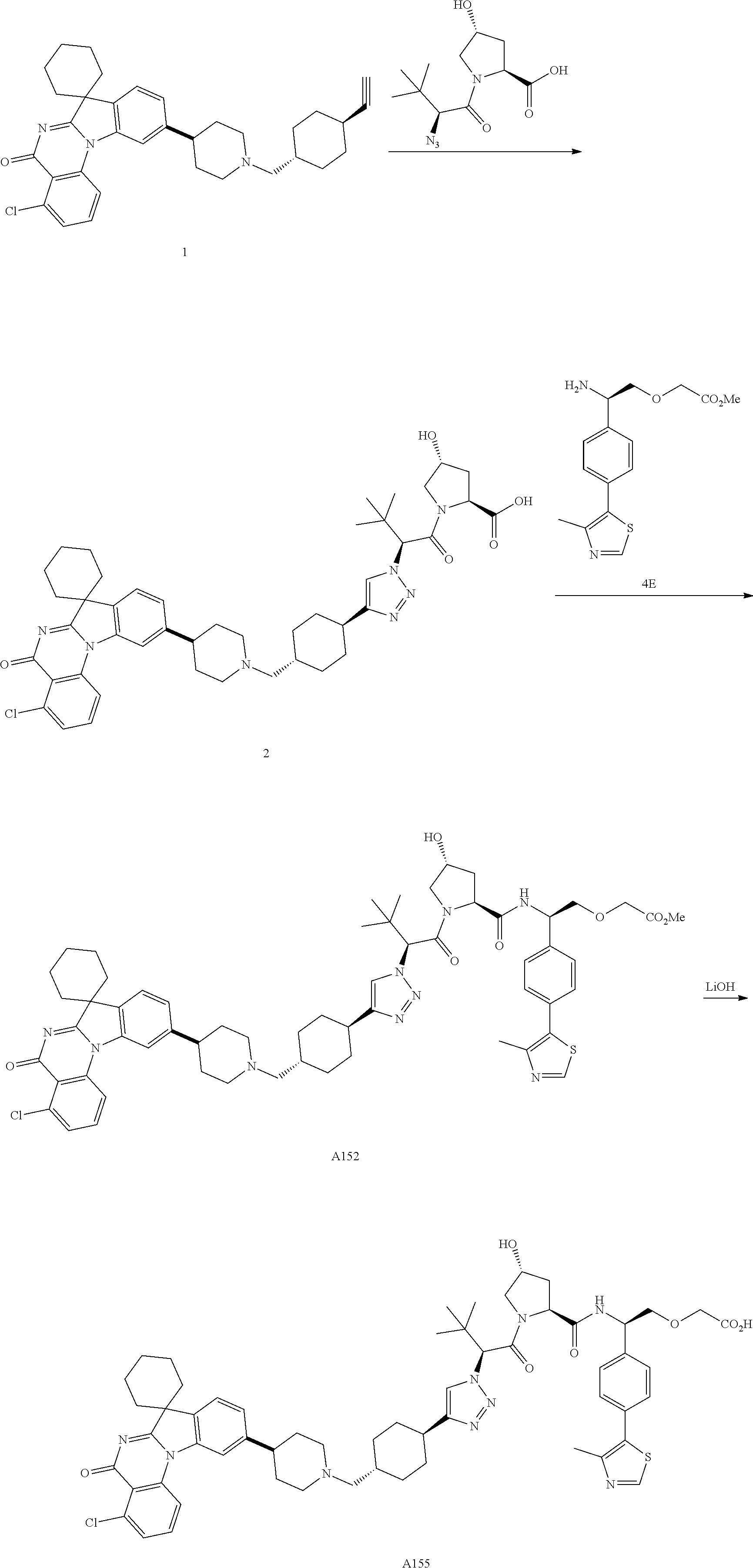

Compound A152 was synthesized using the similar procedures as used in the synthesis of Compound A67. ESI MS m/z: 1098.52 [M+H]⁺.

Preparation of A¹⁵⁵. To a solution of A¹⁵² (30 mg) in MeOH/THF/H₂O (1/1/1, 4 mL) was added LiOH (30 mg) The mixture was stirred for 3 h. The reaction was quenched by TFA.

The mixture was subjected to Prep-HPLC (30-100% MeCN in H₂O, 0.1% TFA) to give compound A¹⁵⁵ (23 mg, 77%). ESI MS m/z: 1084.46 [M+H]⁺.

### Compound A153. methyl 1-((R)-2-((2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxypyrrolidine-2-carboxamido)-2-(4-(4-methylthiazol-5-yl)phenyl)ethyl)piperidine-4-carboxylate

### Compound A156. 1-((R)-2-((2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxypyrrolidine-2-carboxamido)-2-(4-(4-methylthiazol-5-yl)phenyl)ethyl)piperidine-4-carboxylic acid

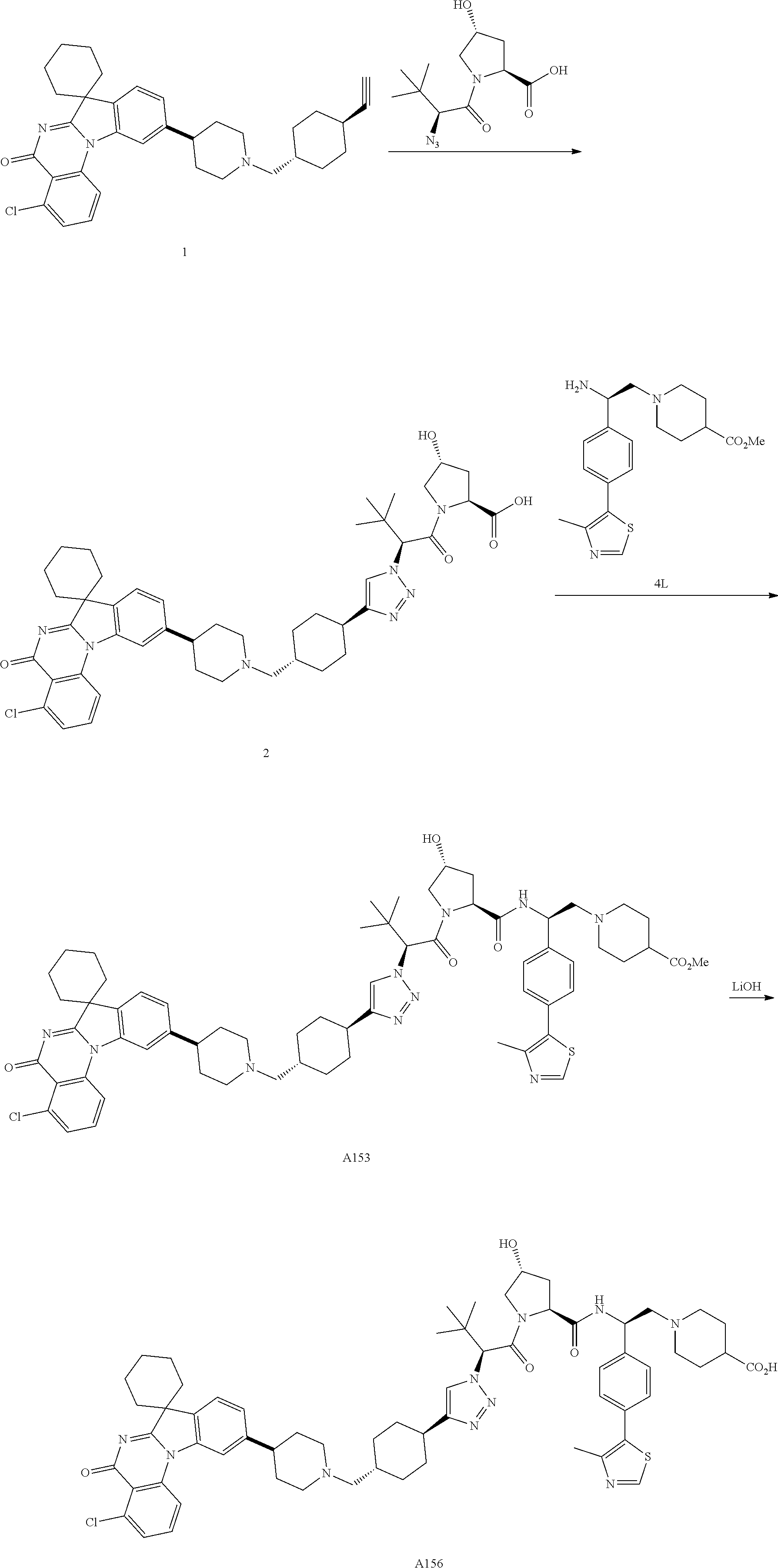

Compound A153 was synthesized using the similar procedures as used in the synthesis of Compound A67. ESI MS m/z: 1151.59 [M+H]⁺.

Compound A156. To a solution of A¹⁵³ (15 mg) in MeOH/THF/H₂O (1/1/1, 3 mL) was added LiOH (15 mg) The mixture was stirred for 3 h. The reaction was quenched by TFA. The mixture was subjected to Prep-HPLC (30-100% MeCN in H₂O, 0.1% TFA) to give compound A¹⁵⁶ (9.1 mg, 61%). ESI MS m/z: 1137.55 [M+H]⁺.

### Compound A154. methyl 1-((R)-2-((2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxypyrrolidine-2-carboxamido)-2-(4-(4-methylthiazol-5-yl)phenyl)ethyl) azetidine-3-carboxylate

### Compound A157. 1-((R)-2-((2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxypyrrolidine-2-carboxamido)-2-(4-(4-methylthiazol-5-yl)phenyl)ethyl) azetidine-3-carboxylic acid

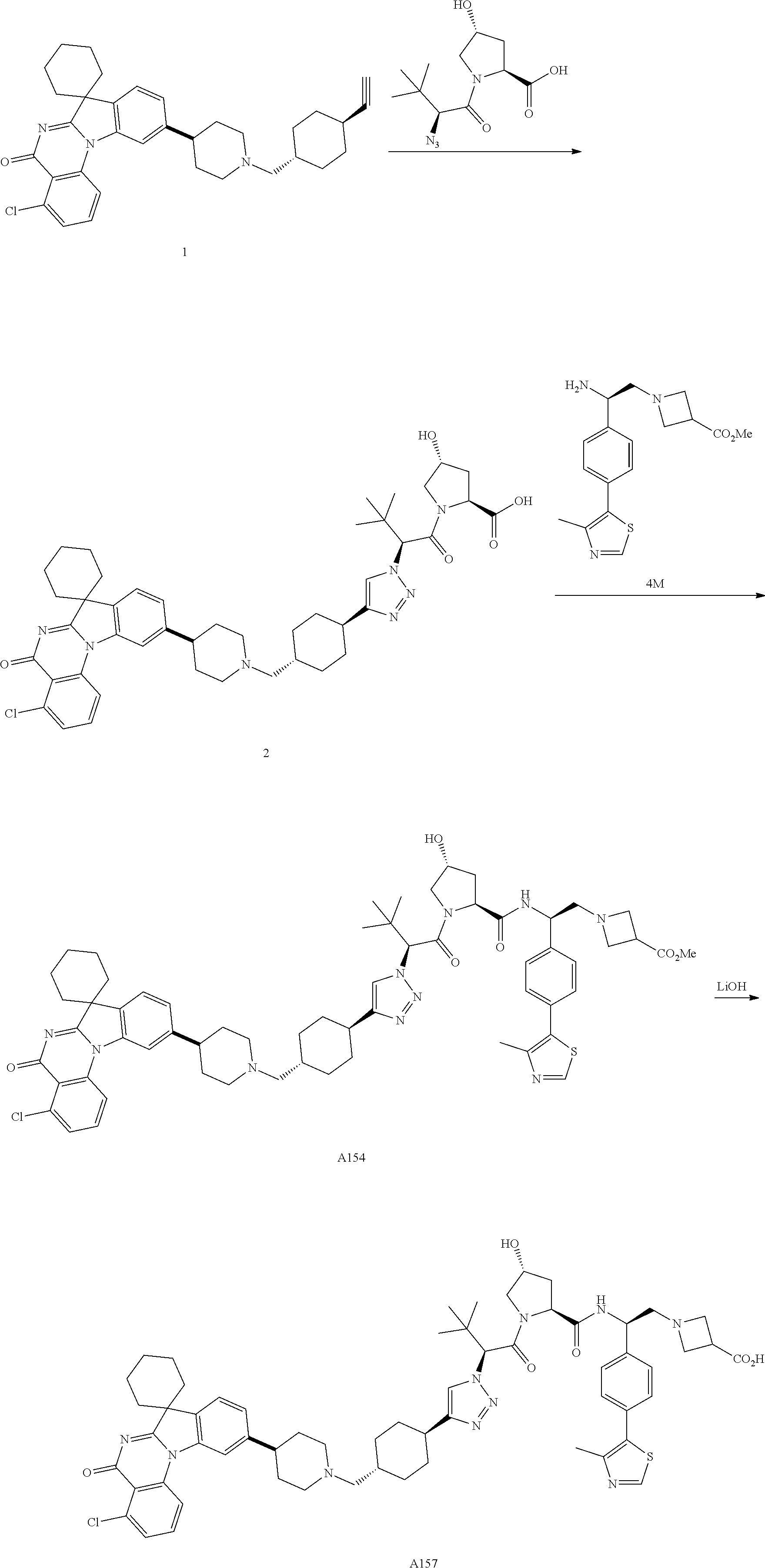

Compound A154 was synthesized using the similar procedures as used in the synthesis of Compound A67. ESI MS m/z: 1123.69 [M+H]⁺.

Compound A157. To a solution of A¹⁵⁴ (14 mg) in MeOH/THF/H₂O (1/1/1, 3 mL) was added LiOH (14 mg) The mixture was stirred for 3 h. The reaction was quenched by TFA. The mixture was subjected to Prep-HPLC (30-100% MeCN in H₂O, 0.1% TFA) to give compound A¹⁵⁷ (7.9 mg, 56%). ESI MS m/z: 1108.66 [M+H]⁺.

Compound A158 was synthesized using the similar procedures as used in the synthesis of Compound A100. ESI MS m/z: 1097.59 [M+H]⁺.

Compound A159 was synthesized using the similar procedures as used in the synthesis of Compound A100. ESI MS m/z: 1057.74 [M+H]⁺.

Compound A160 was synthesized using the similar procedures as used in the synthesis of Compound A100. ESI MS m/z: 1141.86 [M+H]⁺.

### Synthesis of Compound 4A, 4B, 4C, 4D, 4E, 4F and 4G.

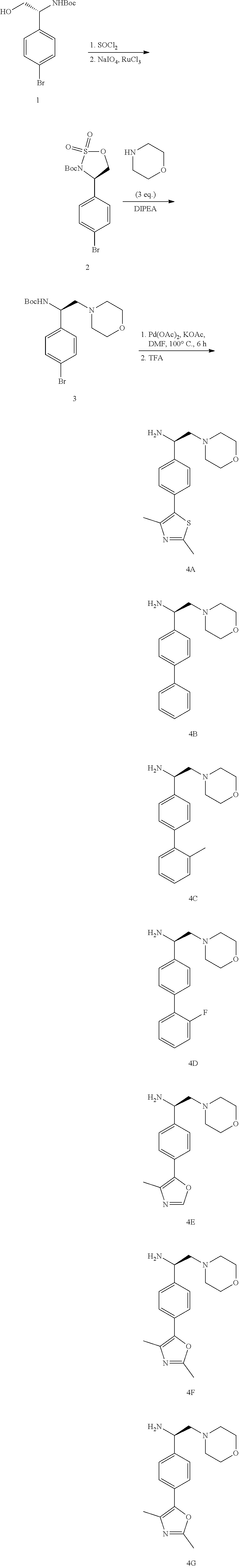

Preparation of 2. The 1 (10 g, 1.0 eq.) in CH₂Cl₂ (100 mL) was added dropwise to a stirred solution of SOCl₂ (1.1 eq.), NEt₃ (2.2 eq.) and imidazole (4 eq.) in CH₂Cl₂ (15 mL) at −78° C. (CO₂ and EtOH) and the resulting solution was stirred at −78° C. for 3 h. The reaction mixture was warmed to room temperature, quenched by addition of water, phases separated, organic phase washed with brine, dried (Na₂SO₄) and concentrated in vacuo to give the crude cyclic sulfamidite. The crude cyclic sulfamidite was dissolved in MeCN (0.13 M) at rt, NaIO4 (1.6 eq) RuCl3 (0.5 mol %) and water (0.16 M) were added sequentially and the resulting solution was stirred until completion was indicated by TLC. The reaction mixture was diluted with water (equal volume to that used in reaction), extracted with Et₂O, washed with brine, dried (Na₂SO₄) and concentrated in vacuo to give the crude product 2 (88% for two steps).

Preparation of 3. To a solution of 2 (5.54 g, 14.6 mmol, 1 equiv.) in CH₃CN (100 mL) was added morpholine (6.4 mL, 73.2 mmol, 5 equiv.). The mixture was stirred at rt for 3 h. After reaction, the solvent was removed, the residue was purified by C-18 column chromatography (elution solvents: ethyl acetate/hexane=1/1) to get pure product 3 (4.27 g, 76%).

Preparation of 4. To a solution of 3 (200 mg, 0.52 mmol, 1 equiv.) and 2,4-dimethylthiazole (0.11 mL, 1.04 mmol, 2.0 equiv.) in DMF (3 mL) was added Pd (OAc) 2 (8.6 mg, 0.05 mmol, 0.1 equiv.) and KOAc (102 mg, 1.04 mmol, 2 equiv). The mixture was stirred for 10 h at 120° C. Water (150 mL) was added to quench the reaction and product was extracted with ethyl acetate twice (100 mL×2). Combined organic phases were washed by brine twice, dried by anhydrous Na₂SO₄. After removing solvent, the received crude product was purified by reverse phase flash chromatography (10-100% MeCN in H₂O, 0.1% TFA) to get pure product. The product was dissolved in DCM (3 mL), was added TFA (3 mL) and the mixture was stirred 30 min. After removing solvent, the crude product 4A was received and can be used in the next step directly. ESI MS m/z: 318.44 [M+H]⁺.

Compound 4B-G was synthesized using the similar procedures as used in the synthesis of Compound 4A.

### Compound A161. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-N—((R)-1-(4-(2,4-dimethylthiazol-5-yl)phenyl)-2-morpholinoethyl)-4-hydroxypyrrolidine-2-carboxamide

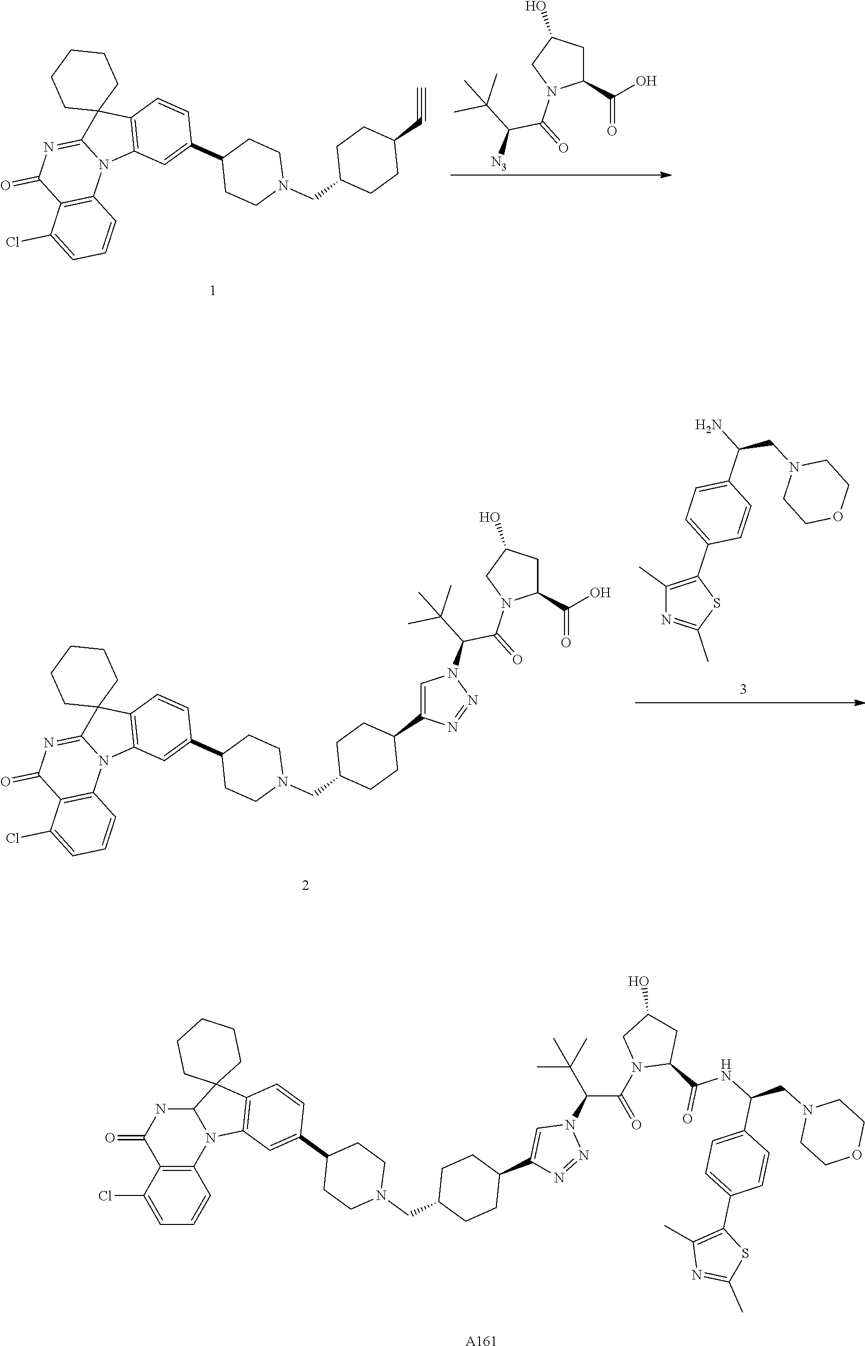

Compound A161 was synthesized using the similar procedures as used in the synthesis of Compound A67. ESI MS m/z: 1109.56 [M+H]⁺.

### Compound A162. (2S,4R)—N—((R)-1-([1,1′-biphenyl]-4-yl)-2-morpholinoethyl)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxypyrrolidine-2-carboxamide

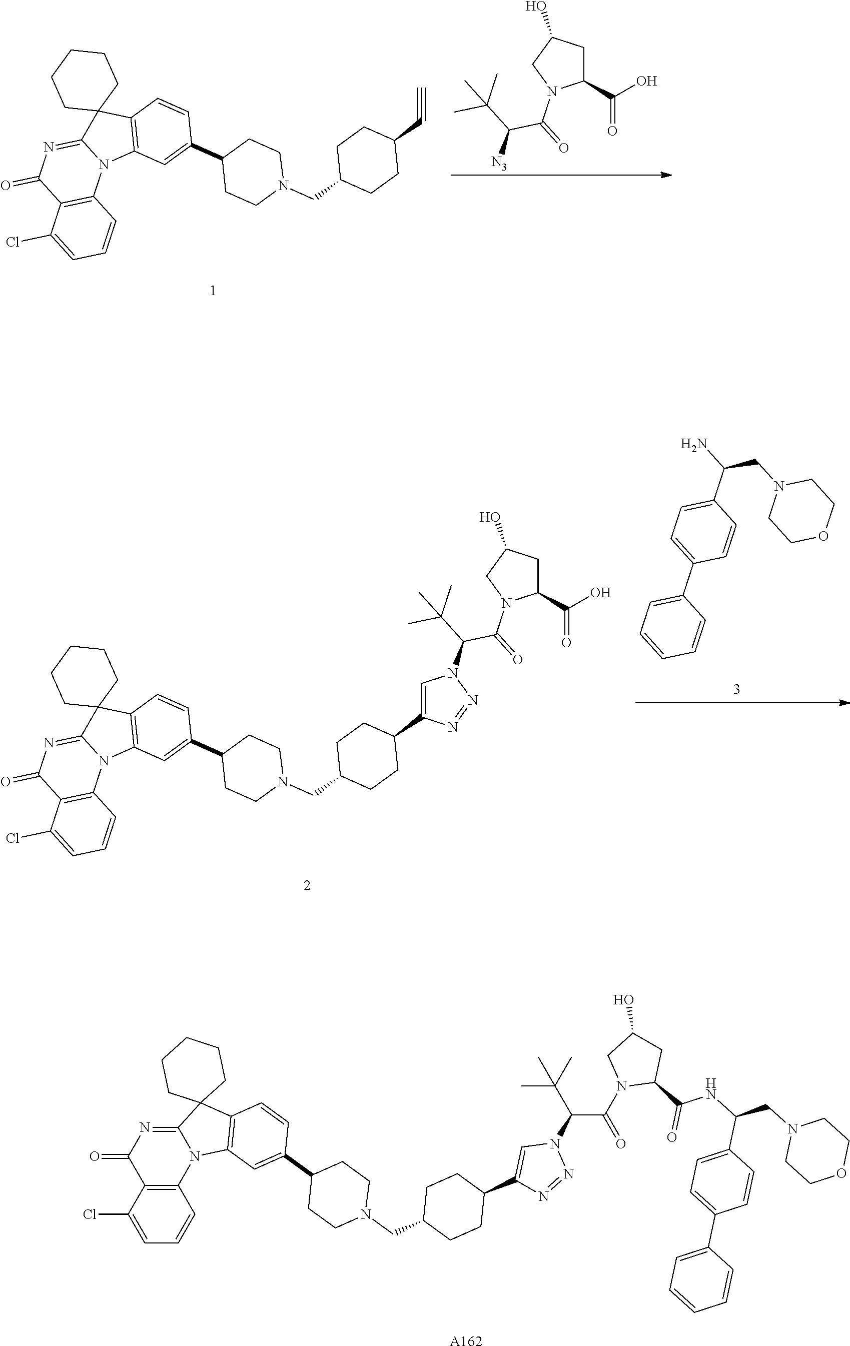

Compound A162 was synthesized using the similar procedures as used in the synthesis of Compound A67. ESI MS m/z: 1074.68 [M+H]⁺.

### Compound A163. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-1-(2′-methyl-[1,1′-biphenyl]-4-yl)-2-morpholinoethyl)pyrrolidine-2-carboxamide

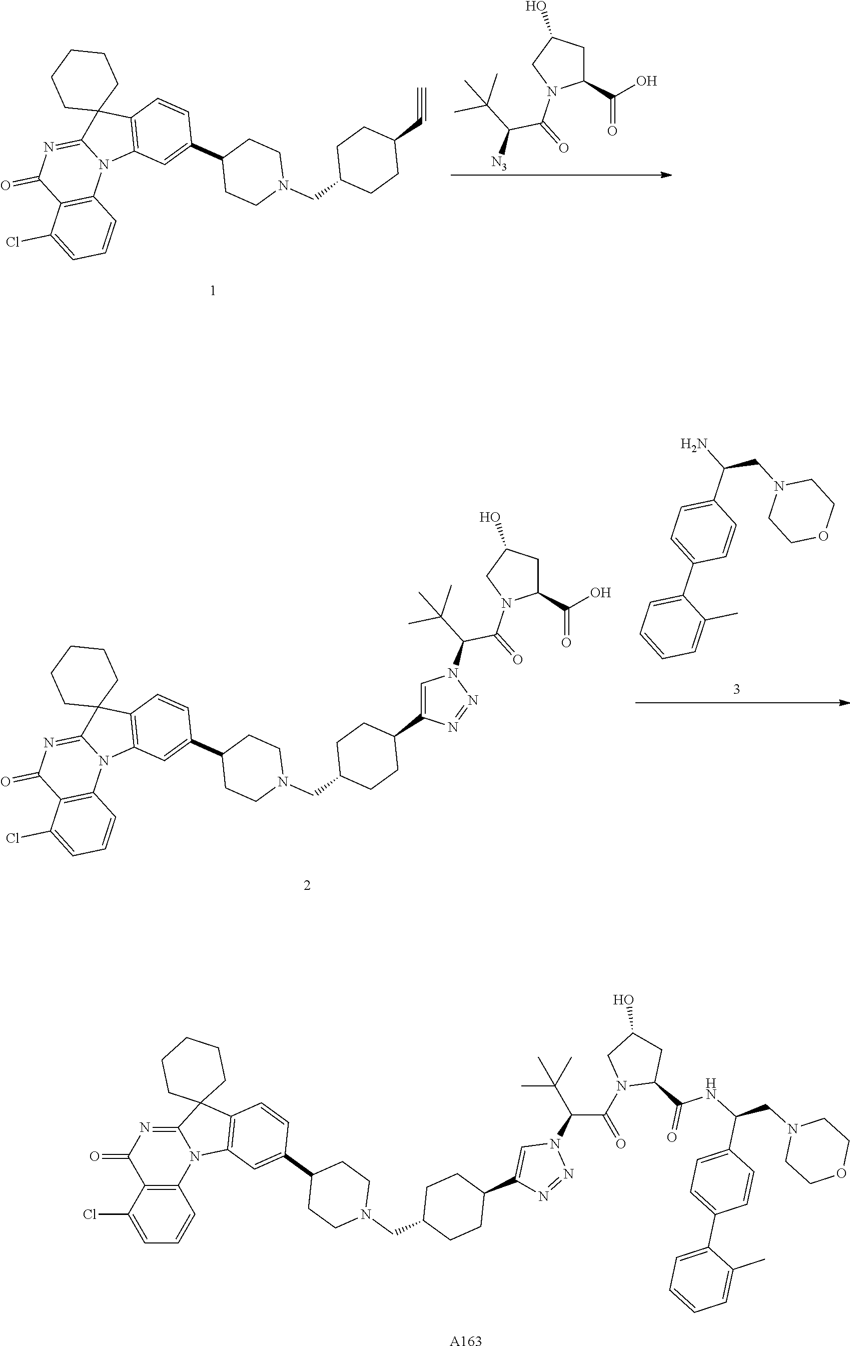

Compound A163 was synthesized using the similar procedures as used in the synthesis of Compound A67. ESI MS m/z: 1088.59 [M+H]⁺.

### Compound A164, (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-N—((R)-1-(2′-fluoro-[1,1′-biphenyl]-4-yl)-2-morpholinoethyl)-4-hydroxypyrrolidine-2-carboxamide

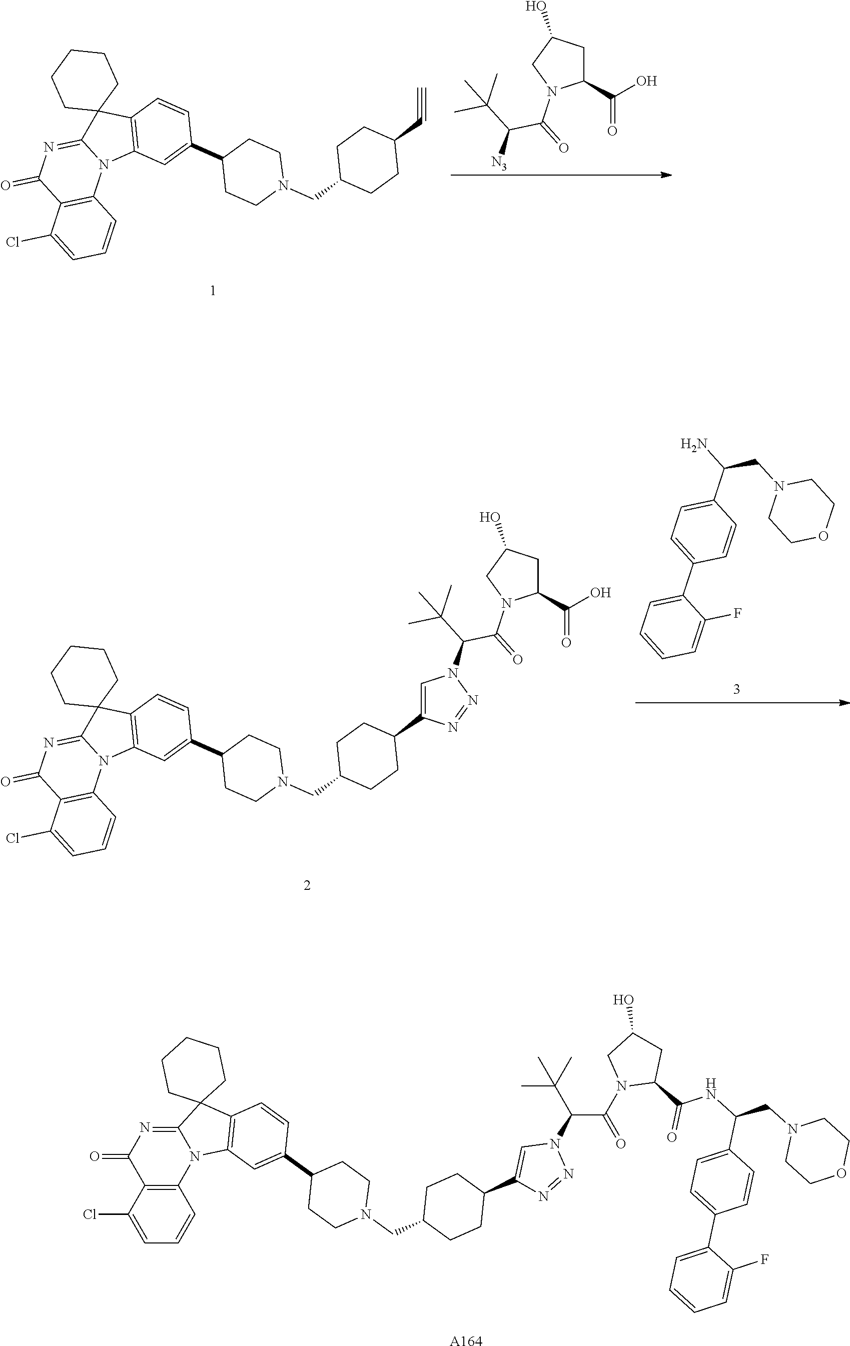

Compound A164 was synthesized using the similar procedures as used in the synthesis of Compound A67. ESI MS m/z: 1091.99 [M+H]⁺.

### Compound A165. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-1-(4-(4-methyloxazol-5-yl)phenyl)-2-morpholinoethyl)pyrrolidine-2-carboxamide

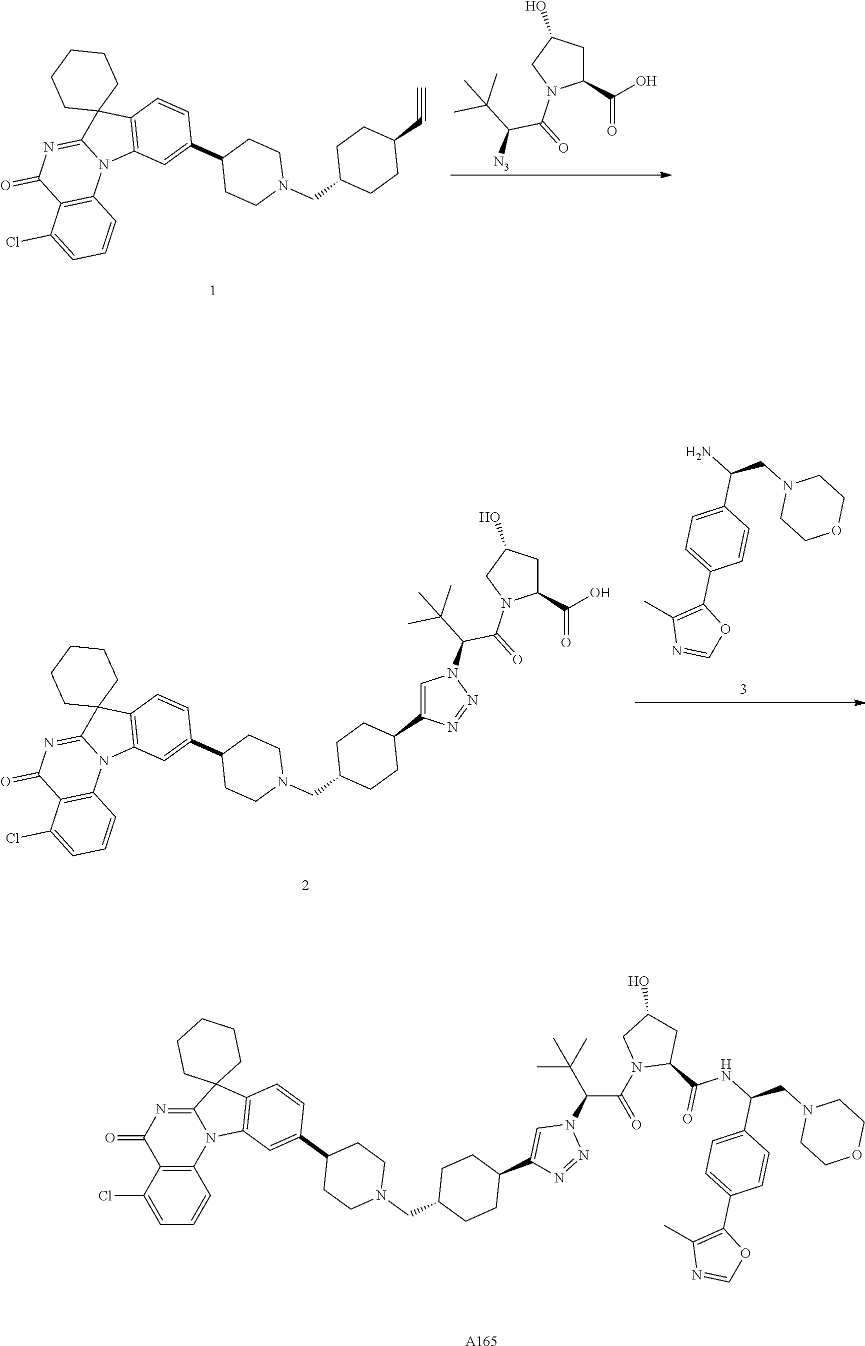

Compound A165 was synthesized using the similar procedures as used in the synthesis of Compound A67. ESI MS m/z: 1079.48 [M+H]⁺.

### Compound A166. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-N—((R)-1-(4-(2,4-dimethyloxazol-5-yl)phenyl)-2-morpholinoethyl)-4-hydroxypyrrolidine-2-carboxamide

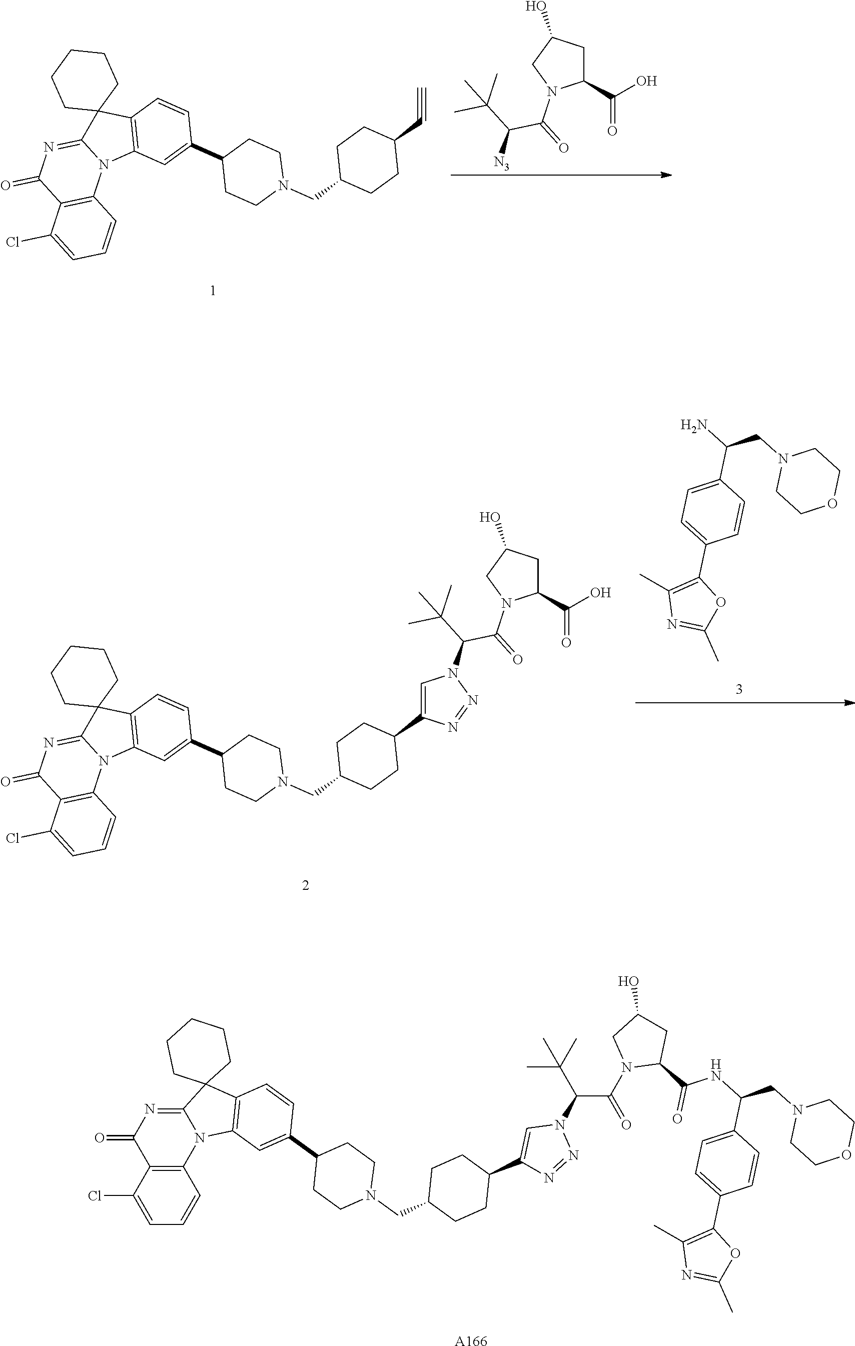

Compound A166 was synthesized using the similar procedures as used in the synthesis of Compound A67. ESI MS m/z: 1093.48 [M+H]⁺.

### Compound A167. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-2-morpholino-1-(4-(4-(trifluoromethyl)thiazol-5-yl)phenyl)ethyl)pyrrolidine-2-carboxamide

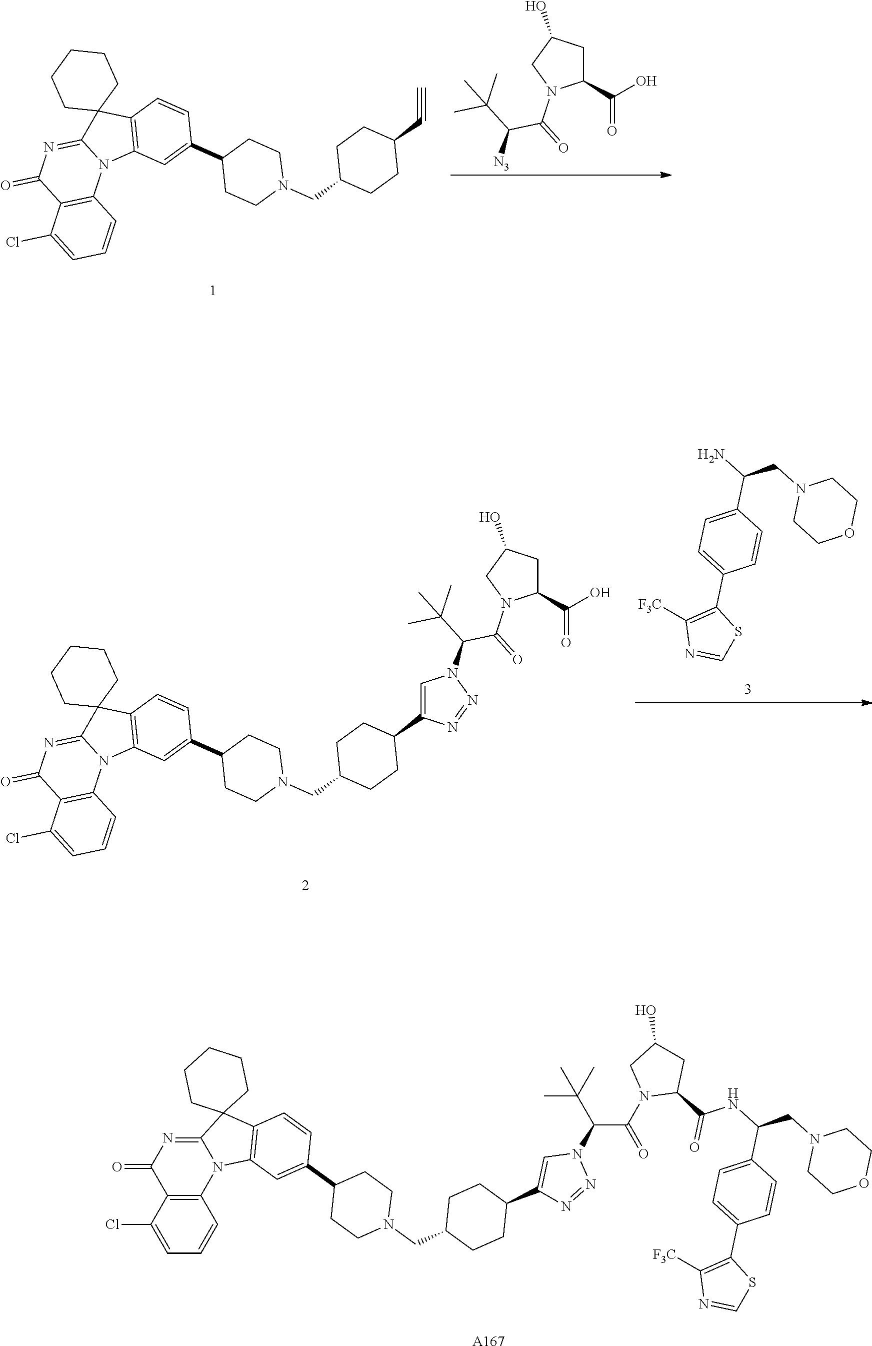

Compound A167 was synthesized using the similar procedures as used in the synthesis of Compound A67. ESI MS m/z: 1048.96 [M+H]⁺.

### Compound A168. (2S,4R)-1-((S)-2-(4-(1-((1-(4′-chloro-5′-oxo-5′H-spiro[cyclohexane-1,7′-indolo[1,2-a]quinazolin]-10′-yl)piperidin-4-yl)methyl)piperidin-4-yl)-1H-1,2,3-triazol-1-yl)-3,3-dimethylbutanoyl)-4-hydroxy-N—((R)-1-(4-(4-methylthiazol-5-yl)phenyl)-2-morpholinoethyl)pyrrolidine-2-carboxamide

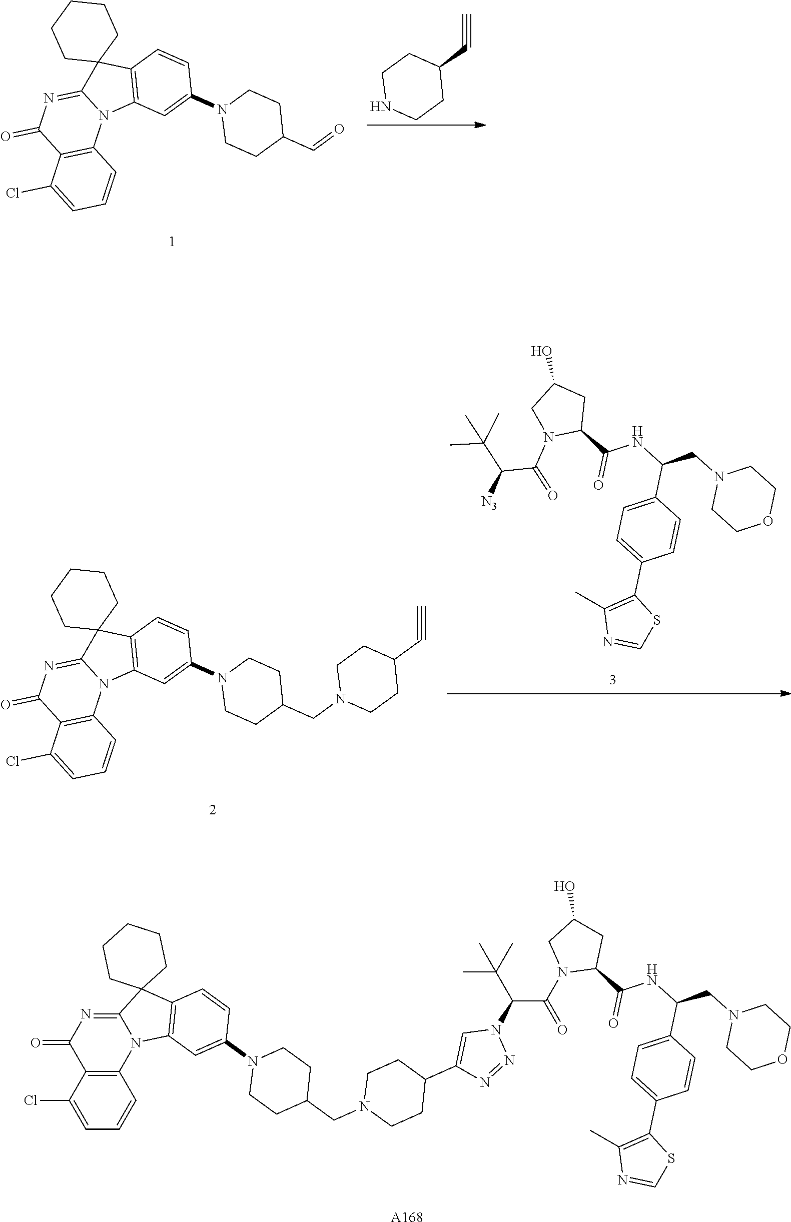

Compound A168 was synthesized using the similar procedures as used in the synthesis of Compound A49. ESI MS m/z: 1095.96 [M+H]⁺.

### Compound A169. (2S,4R)-1-((S)-2-(4-(1-((1-(4′-bromo-5′-oxo-5′H-spiro[cyclohexane-1,7′-indolo[1,2-a]quinazolin]-10′-yl)piperidin-4-yl)methyl)piperidin-4-yl)-1H-1,2,3-triazol-1-yl)-3,3-dimethylbutanoyl)-4-hydroxy-N—((R)-1-(4-(4-methylthiazol-5-yl)phenyl)-2-morpholinoethyl)pyrrolidine-2-carboxamide

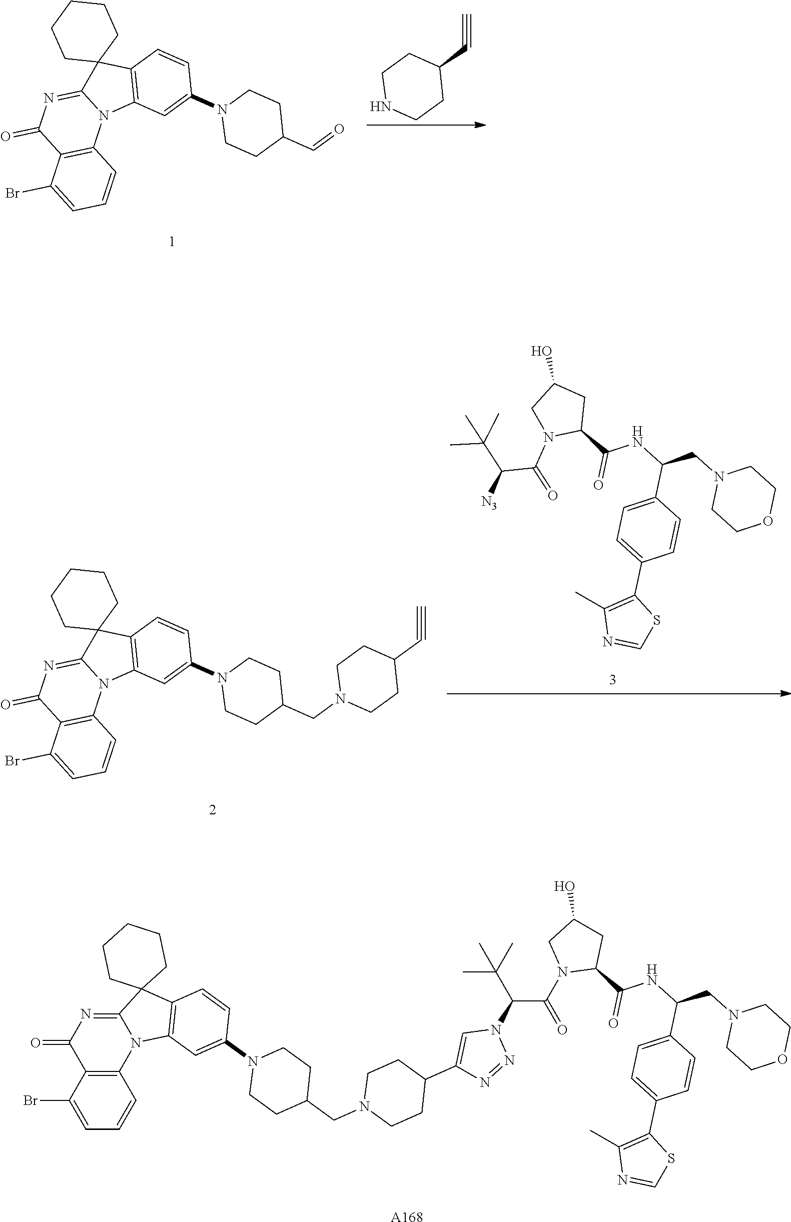

Compound A169 was synthesized using the similar procedures as used in the synthesis of Compound A49. ESI MS m/z: 1140.26 [M+H]⁺.

### Compound A170. (2S,4R)-1-((S)-3,3-dimethyl-2-(4-(4-((4-(5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)butanoyl)-4-hydroxy-N—((R)-1-(4-(4-methylthiazol-5-yl)phenyl)-2-morpholinoethyl)pyrrolidine-2-carboxamide

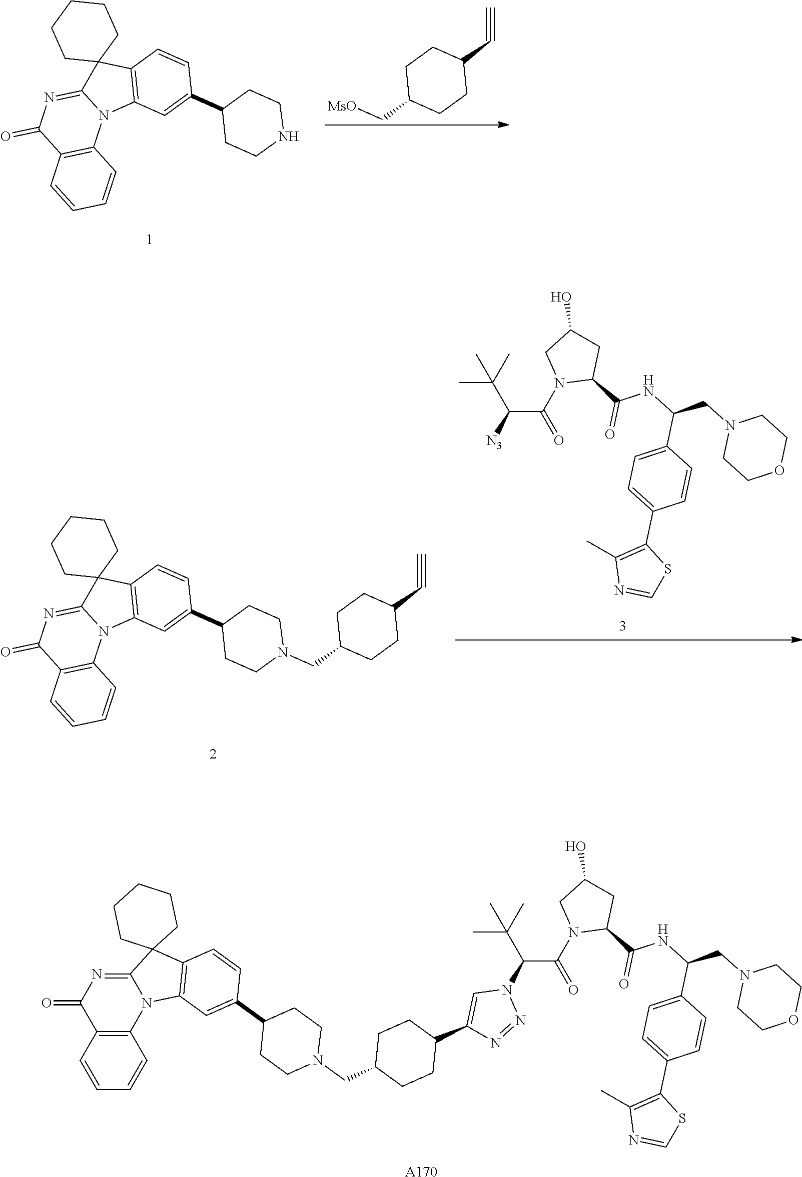

Compound A170 was synthesized using the similar procedures as used in the synthesis of Compound A49. ESI MS m/z: 1061.29 [M+H]⁺.

Compound 171. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-2-morpholino-1-(4-(pyridin-3-yl)phenyl)ethyl)pyrrolidine-2-carboxamide

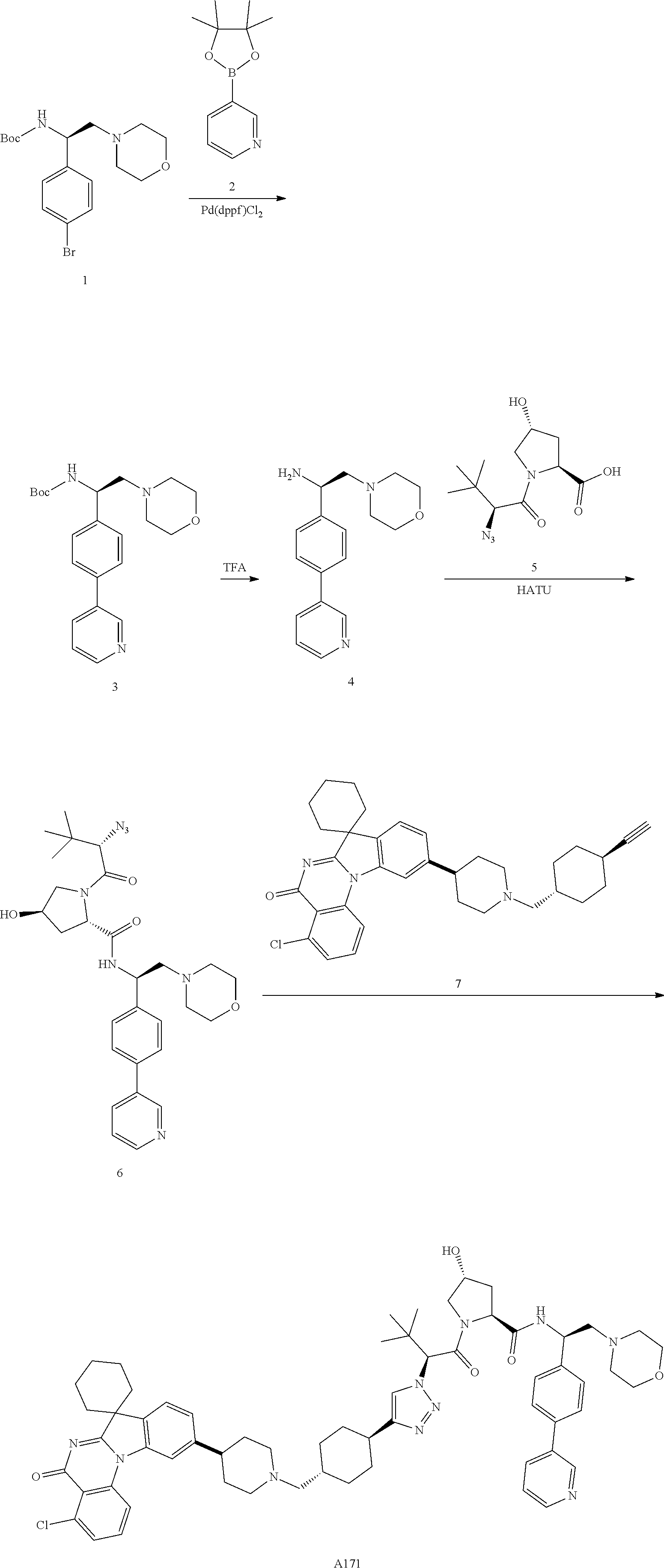

Synthesis of 3, To a solution of 1 (500 mg, 1.30 mmol, 1 equiv.) in dioxane/water (10/2 mL) was added 2 (400 mg, 1.95 mmol, 1.5 equiv.), Pd(dppf)Cl₂ (95 mg, 0.13 mmol, 0.1 equiv.) and K₂CO₃ (358 mg, 2.60 mmol, 2.0 equiv.). The mixture was refluxed for 3 h. after reaction, the solvent was removed, and the residue was dissolved into a mixture of ethyl acetate and water. The organic phase was collected and dried by anhydrous Na₂SO₄. After removed solvent, the residue was purified silica gel column chromatography (elution solvents: EA/hexane=1/1) to get product (230 mg, 46%).

For the next steps, they are similar to the general procedures.

A171 was obtained as white solid. ESI MS m/z: 1075.56 [M+H]⁺.

### Compound 172. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-2-morpholino-1-(4-(pyridin-4-yl)phenyl)ethyl)pyrrolidine-2-carboxamide

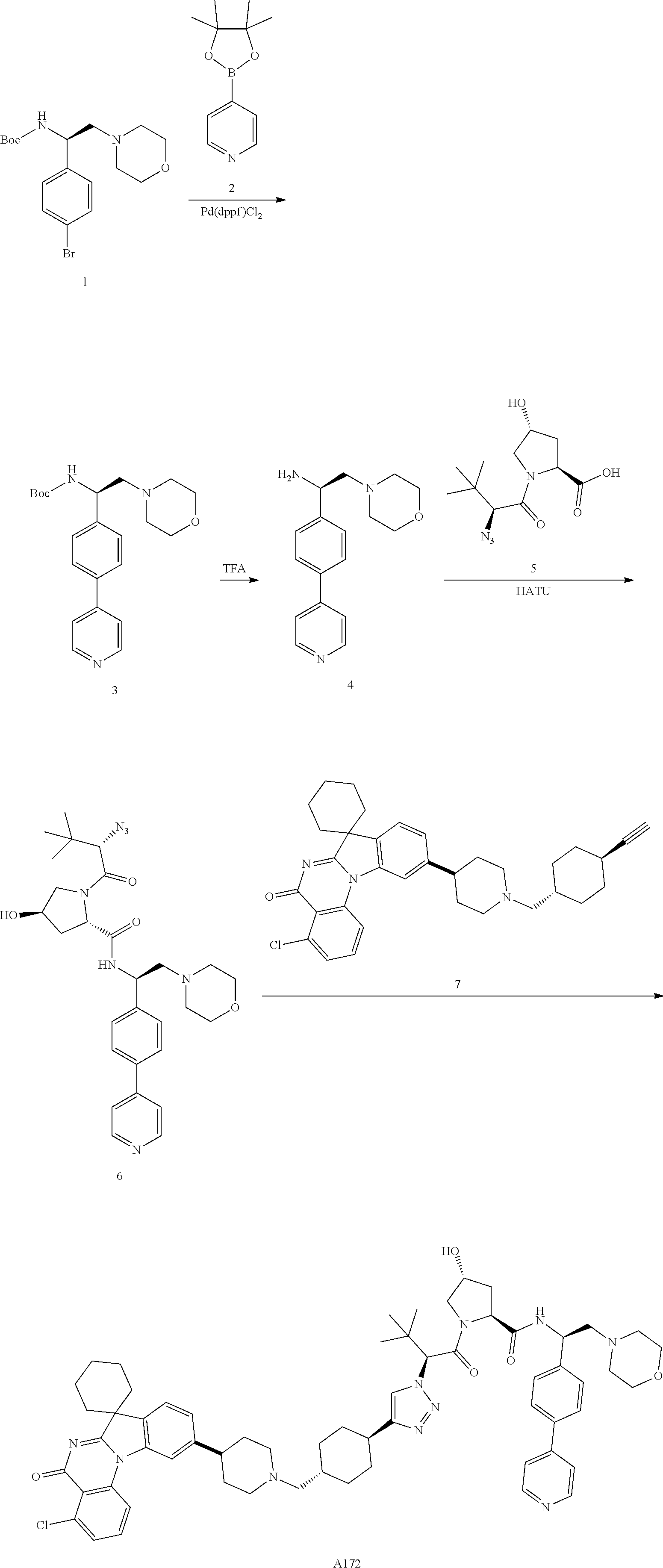

The procedure is similar to the general procedures of synthesis of A¹⁷¹. A¹⁷² was obtained as white solid. ESI MS m/z: 1075.56 [M+H]⁺.

### Compound 173. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-2-morpholino-1-(4-(pyridin-2-yl)phenyl)ethyl)pyrrolidine-2-carboxamide

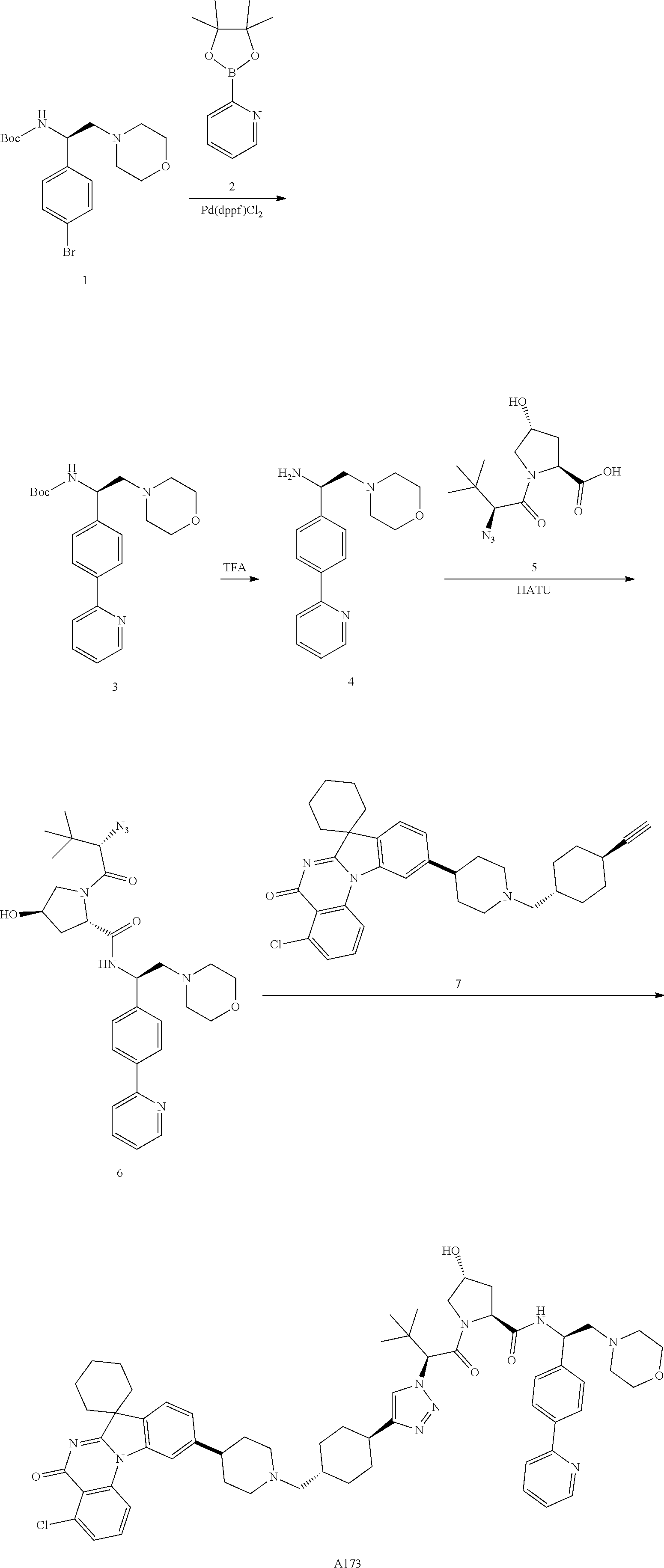

The procedure is similar to the general procedures of synthesis of A¹⁷¹. A¹⁷³ was obtained as white solid. ESI MS m/z: 1075.56 [M+H]⁺.

### Compound 174. (2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-1-(4-(2-methylpyridin-3-yl)phenyl)-2-morpholinoethyl)pyrrolidine-2-carboxamide

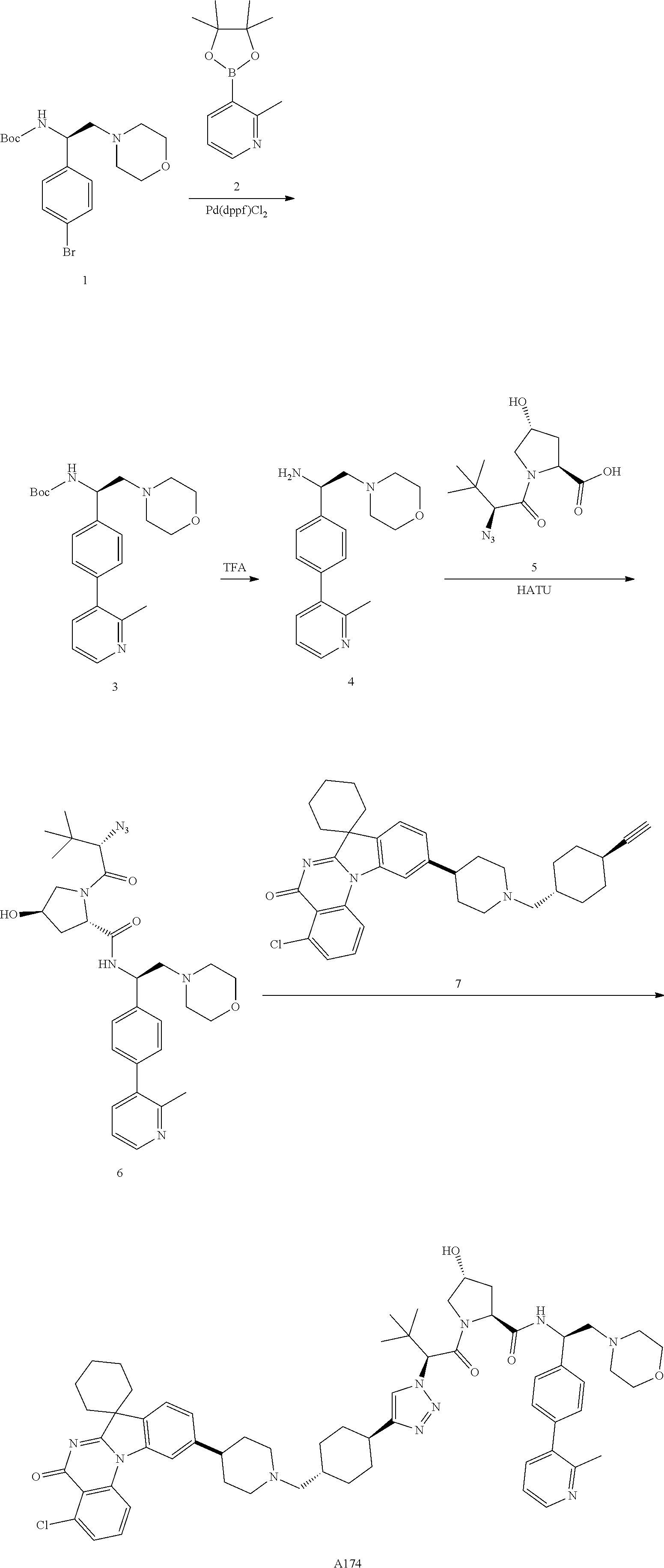

The procedure is similar to the general procedures of synthesis of A¹⁷¹. A¹⁷⁴ was obtained as white solid. ESI MS m/z: 1089.56 [M+H]⁺.

### Compound 175. ((2S,4R)-1-((S)-2-(4-((1r,4S)-4-((4-(4′-chloro-5′-oxo-5′H-spiro[cyclohexane-1,7′-indolo[1,2-a]quinazolin]-10′-yl)piperidin-1-yl)methyl)cyclohexyl)-1H-1,2,3-triazol-1-yl)-3,3-dimethylbutanoyl)-4-hydroxy-N—((R)-1-(4-(4-methylpyridin-3-yl)phenyl)-2-morpholinoethyl)pyrrolidine-2-carboxamide

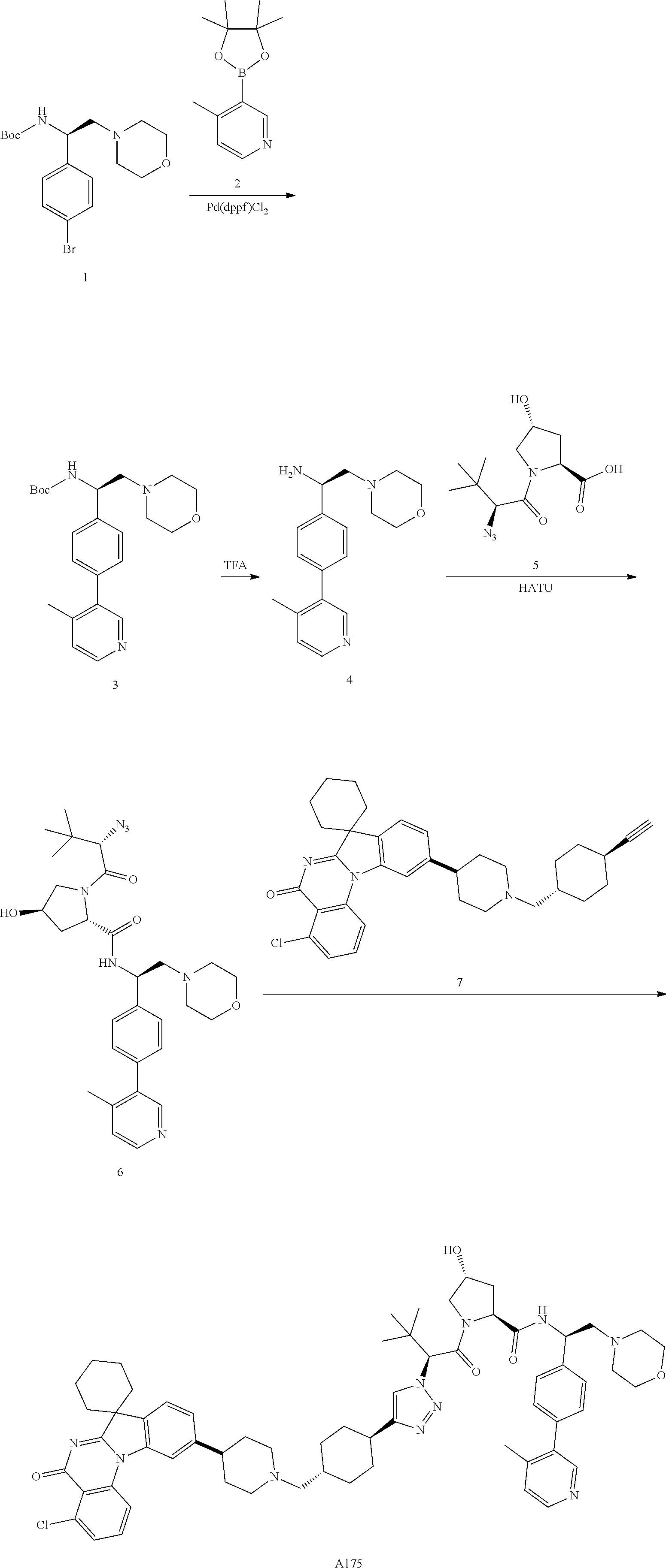

The procedure is similar to the general procedures of synthesis of A¹⁷¹. A¹⁷⁵ was obtained as white solid. ESI MS m/z: 1089.58 [M+H]⁺.

### Compound B1. (2S,4R)-1-((S)-2-((1r,4S)-4-((4-(4-chloro-7,7-dimethyl-5-oxo-5,7-dihydroindolo[1,2-a]quinazolin-9-yl)piperidin-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide

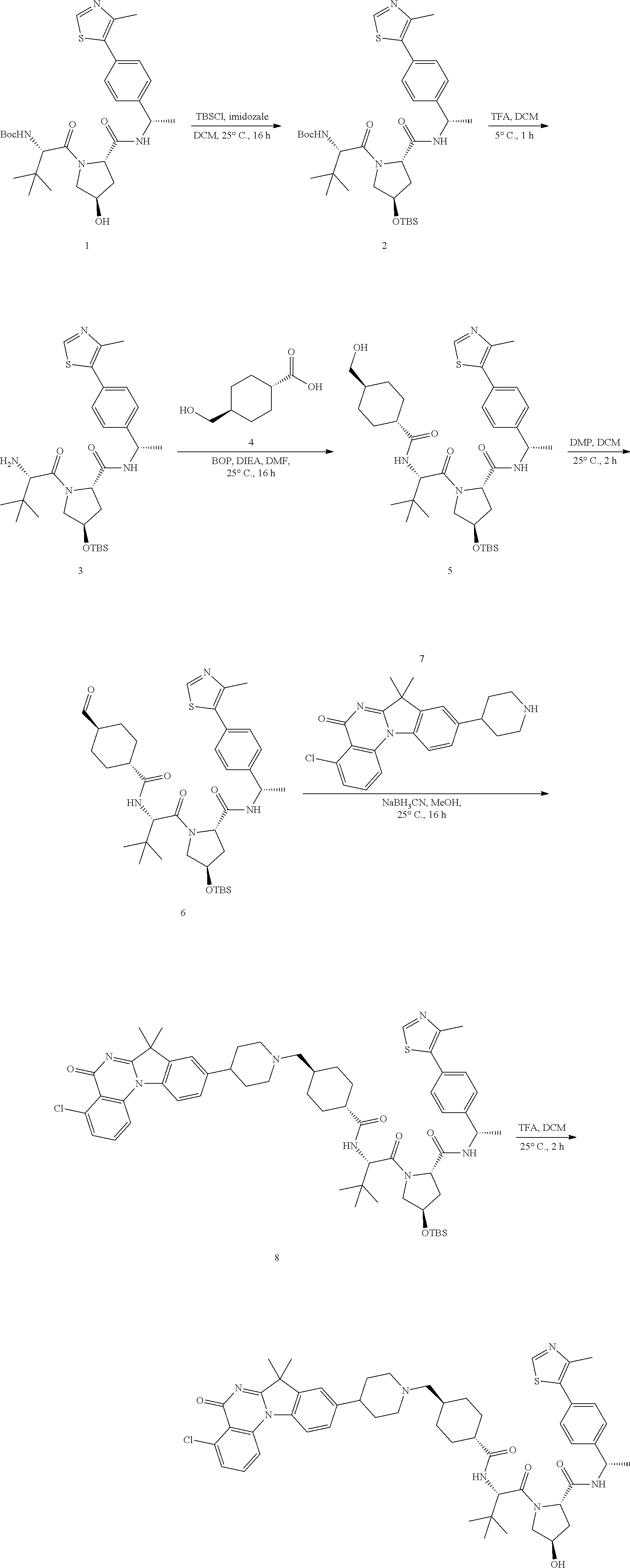

Synthesis of 2, To a solution of 1 (2.00 g, 1 eq, 3.67 mmol) and imidazole (1.00 g, 4 eq, 14.7 mmol) in DCM (15 mL) was added TBS-C₁ (1.66 g, 3 eq, 11.0 mmol). The solution was stirred at 25° C. for 16 h. LCMS showed the reaction was complete. The reaction mixture was diluted with water (100 mL), extracted with EtOAc (80 mL×2). The organic layer was washed with brine, dried over anhydrous Na₂SO₄, filtered and concentrated under vacuum. The resulting crude product was purified by silica gel chromatography (100-200 mesh silica gel, 50% of EtOAc in PE) to obtain 2 (1.69 g, 2.56 mmol, 69.8%) as a white solid. LC purity (0.1% TFA): 100% (UV at 254 nm)/MS: 659.4 [M+1]; Retention time: 1.67 min.

Synthesis of 3, To a solution of 2 (1.54 g, 1 eq, 2.34 mmol) in DCM (20 mL) was added TFA (8 mL). The mixture was stirred at 5° C. for 1 h. LCMS showed the reaction was complete. To the mixture was added aqueous sodium bicarbonate (100 mL) and extracted with EtOAc (60 mL×3). The combined organic layer was dried over sodium sulfate, filtered and concentrated under vacuum to give 3 (1.20 g, 2.15 mmol, 91.9%) as a yellow solid. LC purity (0.1% FA): 71% (UV at 254 nm)/MS: 559.4 [M+1]; Retention time: 1.13 min.

Synthesis of 5, To a solution of 3 (860 mg, 1 eq, 1.54 mmol) in DMF (15.0 mL) were added (1r,4r)-4-(hydroxymethyl)cyclohexane-1-carboxylic acid 4 (487 mg, 2 eq, 3.08 mmol), N-ethyl-N-isopropylpropan-2-amine (597 mg, 3 eq, 4.62 mmol) and BOP (3.40 g, 5 eq, 7.69 mmol). The mixture was stirred at 25° C. for 16 h. LCMS showed the reaction was complete. The reaction mixture was diluted with water (100 mL) and extracted with EtOAc (150 mL×2). The combined organic layer was washer with brine, dried over anhydrous sodium sulfate, filtered and concentrated under vacuum. The resulting crude product was purified by silica gel chromatography (100-200 mesh silica gel, 30% of EtOAc in PE) to 5 (750 mg, 1.07 mmol, 69.7%) as a yellow oil. LC purity (0.1% TFA): 100% (UV at 254 nm)/MS: 721.4 [M+23]; Retention time: 2.21 min.

Synthesis of 6, To a solution of 5 (100.00 mg, 0.144 mmol, 1 eq) in DCM (5 mL) was added DMP (121.00 mg, 0.288 mmol, 2 eq) at 0° C. under Argon atmosphere. The reaction was stirred at 25° C. for 2 h. LCMS and TLC showed the reaction was complete. The mixture was diluted with water (10 mL) and extracted with EtOAc (10 mL×3). The combined organic layer was dried over sodium sulfate, filtered and concentrated under vacuum. The resulting residue was purified by silica gel column chromatography (100-200 mesh size, 30% of EtOAc in PE) to afford 6 (80.0 mg, 0.044 mmol, 80.2% yield), as a yellow oil. LC purity (0.1% FA): 60.08% (UV at 254 nm)/MS: 697.4 [M+1]; Retention time: 1.81 min

Synthesis of 8, To a solution of 6 (80.00 mg, 0.115 mmol, 1.5 eq) and 4-chloro-7,7-dimethyl-9-(piperidin-4-yl)indolo[1,2-a]quinazolin-5 (7H)-one 7 (30.00 mg, 0.079 mmol, 1 eq) in MeOH (5 mL) was added NaBH₃CN (66.00 mg, 0.789 mmol, 10 eq) at 25° C. under Argon atmosphere. The reaction was stirred at 25° C. for 16 h. LCMS and TLC showed the reaction was complete. The mixture was diluted with water (10 mL) and extracted with DCM (10 mL×3). The combined organic layer was dried over sodium sulfate, filtered and concentrated under vacuum. The resulting residue was purified by silica gel column chromatography (100-200 mesh size, 10% of DCM in MeOH) to afford 8 (50.0 mg, 0.047 mmol, 59.7% yield). as a white solid. LC purity (0.1% FA): 61.73% (UV at 254 nm)/MS: 1061.6 [M+1]; Retention time: 1.53 min

### Synthesis of (2S,4R)-1-((S)-2-((1r,4S)-4-((4-(4-chloro-7,7-dimethyl-5-oxo-5,7-dihydroindolo[1,2-a]quinazolin-9-yl)piperidin-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide

To a solution of (2R,4S)-4-((tert-butyldimethylsilyl)oxy)-1-((S)-2-((1r,4S)-4-((4-(4-chloro-7,7-dimethyl-5-oxo-5,7-dihydroindolo[1,2-a]quinazolin-9-yl)piperidin-1-yl)methyl)cyclohexane-1-carboxamido)-3,3-dimethylbutanoyl)-N—((S)-1-(4-(4-methylthiazol-5-yl)phenyl)ethyl)pyrrolidine-2-carboxamide 8 (50.0 mg, 0.047 mmol, 1 eq) in DCM (10 mL) was added TFA (2.5 mL, 0.44 mmol, 10 eq). The reaction was stirred at 25° C. for 2 h. LCMS and TLC showed the reaction was complete. The reaction mixture was concentrated under vacuum. The residue was purified by prep-HPLC, eluted with 5% to 95% acetonitrile in water (containing 0.03% ammonium carbonate) to afford (2S,4R)-1-((S)-2-((1r,4S)-4-((4-(4-chloro-7,7-dimethyl-5-oxo-5,7-dihydroindolo[1,2-a]quinazolin-9-yl)piperidin-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide Compound B1 (4.96 mg, 2.47 μmol, 10.7%, 94.73% Purity) as a yellow solid. LC purity (0.1% NH₃·H₂O): 96.38% (UV at 254 nm)/MS: 946.8 [M+1]; Retention time: 1.73 min.

¹H NMR (400 MHz, CD₃OD) δ 8.87 (m, 1H), 8.42 (m, 1H), 8.07 (m, 1H), 7.84 (m, 1H), 7.67-7.52 (m, 2H), 7.42 (m, 5H), 5.00 (m, 2H), 4.56 (m, 2H), 4.43 (s, 1H), 3.86 (m, 1H), 3.75 (m, 1H), 3.13 (s, 2H), 2.72 (s, 1H), 2.48 (s, 3H), 2.36 (s, 2H), 2.24-2.15 (m, 2H), 1.96 (m, 2H), 1.91 (m, 6H), 1.85-1.77 (m, 1H), 1.61 (s, 6H), 1.51 (m, 4H), 1.28 (s, 1H), 1.09 (s, 1H), 1.04 (s, 9H), 1.01 (s, 2H).

### Compound B2. (2S,4R)—N—((S)-6-(4-(4-bromo-7,7-dimethyl-5-oxo-5,7-dihydroindolo[1,2-a]quinazolin-9-yl)piperidin-1-yl)-1-(4-(4-methylthiazol-5-yl)phenyl) hexyl)-1-((S)-2-(1-fluorocyclopropane-1-carboxamido)-3,3-dimethylbutanoyl)-4-hydroxypyrrolidine-2-carboxamide

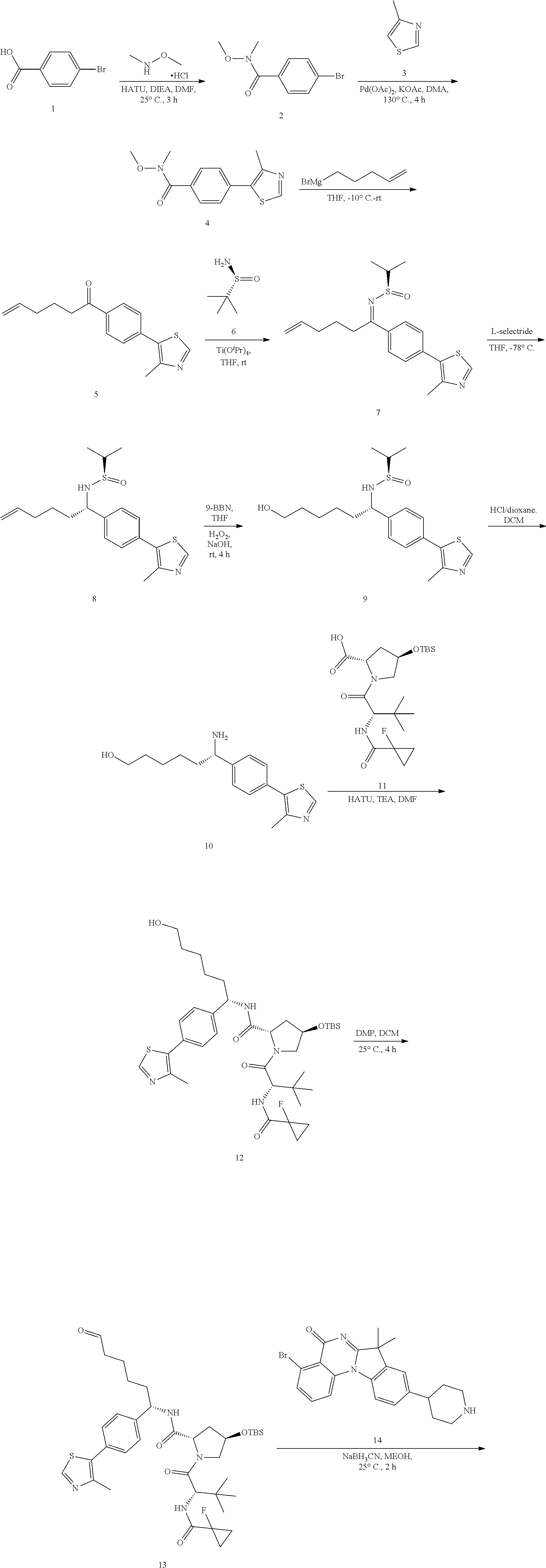

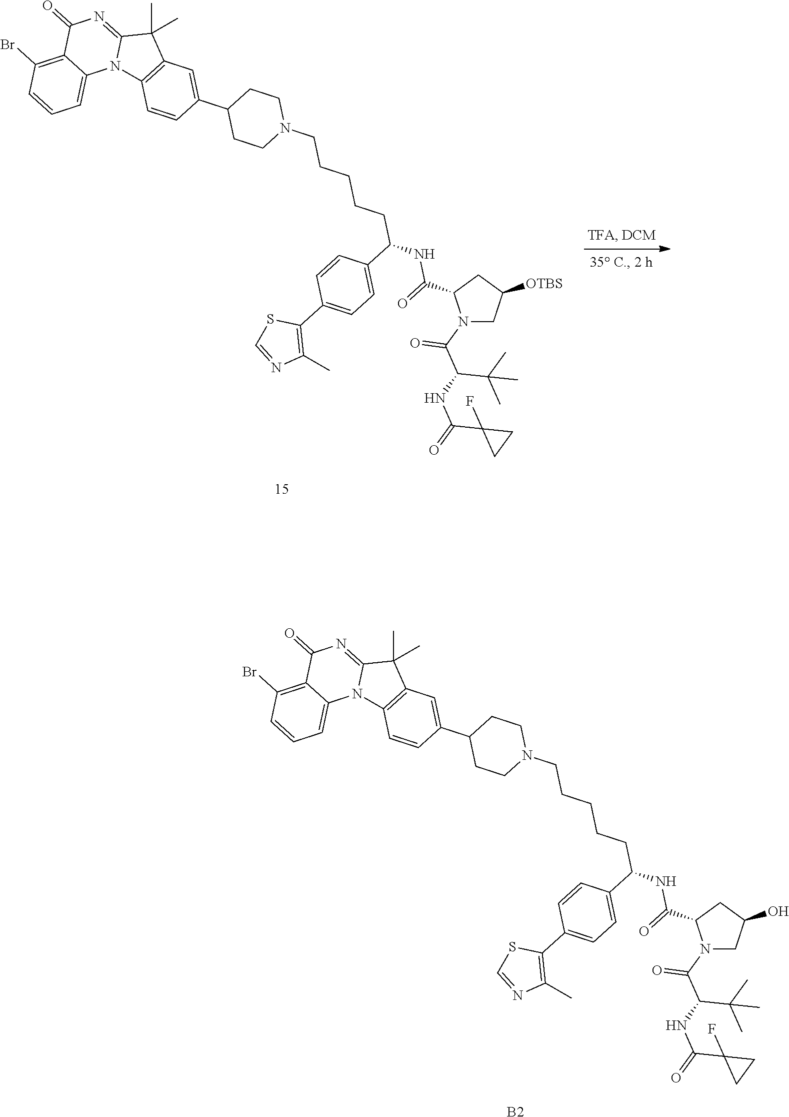 [SMILES: Cc1ncsc1-c1ccc([C@H](CCCCCN2CCC(c3ccc4c(c3)C(C)(C)c3nc(=O)c5c(Br)cccc5n3-4)CC2)NC(=O)[C@@H]2C[C@@H](O[Si](C)(C)C(C)(C)C)CN2C(=O)[C@@H](NC(=O)C2(F)CC2)C(C)(C)C)cc1 ; "15" ; Cc1ncsc1-c1ccc([C@H](CCCCCN2CCC(c3ccc4c(c3)C(C)(C)c3nc(=O)c5c(Br)cccc5n3-4)CC2)NC(=O)[C@@H]2C[C@@H](O)CN2C(=O)[C@@H](NC(=O)C2(F)CC2)C(C)(C)C)cc1 ; "B2"]

### Synthesis of 4-bromo-N-methoxy-N-methylbenzamide (Intermediate 2)

To a solution of 4-bromobenzoic acid (30.0 g, 1 eq, 149 mmol) in DMF (300 mL) was added DIEA (96.4 g, 130 mL, 5 Eq, 746 mmol), HATU (68.1 g, 1.2 Eq, 179 mmol), N,O-dimethylhydroxylamine (10.9 g, 1.2 Eq, 179 mmol). The mixture was stirred at 25° C. for 3 hours. LCMS indicated completion of reaction. The reaction was poured into H₂O (100.0 mL) and extracted with EA (200.0 mL), then washed with brine (100.0 mL) and dried over sodium sulfate, filtered and concentrated under vacuum. The resulting residue was purified by silica gel chromatography (100-200 mesh silica gel, 50% of EtOAc in PE) to afford 4-bromo-N-methoxy-N-methylbenzamide (30.0 g, 0.11 mol, 76%, 92% Purity) as a yellow oil. LC purity (0.1% TFA): 95% (UV at 254 nm)/MS: 245 [M+H]; Retention time: 1.32 min.

### Synthesis of N-methoxy-N-methyl-4-(4-methylthiazol-5-yl)benzamide (Intermediate 4)

To a solution of 4-bromo-N-methoxy-N-methylbenzamide (30.0 g, 1 Eq, 123.0 mmol) in DMA (50.0 mL) was added 4-methylthiazole (24.4 g, 2 Eq, 246.0 mmol), Potassium acetate (36.2 g, 23.0 mL, 3 Eq, 369.0 mmol), Palladium diacetate (478 mg, 0.1 Eq, 2.13 mmol). The mixture was stirred at 130° C. for 4 hours under N₂. LCMS indicated completion of reaction. The reaction was poured into H₂O (100.0 mL) and extracted with EA (200.0 mL), then washed with brine (100.0 mL) and dried over sodium sulfate, filtered and concentrated under vacuum. The resulting residue was purified by silica gel chromatography (100-200 mesh silica gel, 30% of EtOAc in PE) to afford N-methoxy-N-methyl-4-(4-methylthiazol-5-yl)benzamide (21.0 g, 76 mmol, 62%, 95% Purity) as a yellow solid. LC purity (0.1% TFA): 96% (UV at 254 nm)/MS: 263.1 [M+H]; Retention time: 1.24 min.

### Synthesis of 1-(4-(4-methylthiazol-5-yl)phenyl) hex-5-en-1-one (Intermediate 5)

To a solution of N-methoxy-N-methyl-4-(4-methylthiazol-5-yl)benzamide (21.0 g, 1 Eq, 80.1 mmol) in 2-THF (50.0 mL) was added pent-4-en-1-ylmagnesium bromide (55.5 g, 4 Eq, 320 mmol). The mixture was stirred at 0° C. for 2 hours. LCMS indicated completion of reaction. The reaction was poured into H₂O (100.0 mL) and extracted with EA (200.0 mL), then washed with brine (100.0 mL) and dried over sodium sulfate, filtered and concentrated under vacuum. The residue was purified by silica gel chromatography (100-200 mesh silica gel, 10% of EtOAc in PE) to afford 1-(4-(4-methylthiazol-5-yl)phenyl) hex-5-en-1-one (8.20 g, 29 mmol, 36%, 96% Purity) as a yellow oil. LC purity (0.1% TFA): 89% (UV at 254 nm)/MS: 272 [M+H]; Retention time: 1.64

### Synthesis of (R,Z)-2-methyl-N-(1-(4-(4-methylthiazol-5-yl)phenyl) hex-5-en-1-ylidene) propane-2-sulfinamide (Intermediate 7)

To a solution of 1-(4-(4-methylthiazol-5-yl)phenyl) hex-5-en-1-one (8.20 g, 1 Eq, 30.2 mmol) in THF (20.0 mL) was added 2-methylpropane-2-sulfinamide (36.6 g, 10 Eq, 302 mmol). The mixture was stirred at 80° C. for 16 hours. LCMS indicated completion of reaction. LCMS indicated completion of reaction. The reaction was poured into H₂O (100.0 mL) and extracted with EA (200.0 mL), then washed with brine (100.0 mL) and dried over sodium sulfate, filtered and concentrated under vacuum. The resulting residue was purified by silica gel chromatography (100-200 mesh silica gel, 20% of EtOAc in PE) to afford (R,Z)-2-methyl-N-(1-(4-(4-methylthiazol-5-yl)phenyl) hex-5-en-1-ylidene) propane-2-sulfinamide (7.90 g, 19 mmol, 64%, 92% Purity) as a yellow solid. LC purity (0.1% FA): 100% (UV at 254 nm)/MS: 375.2 [M+H]; Retention time: 1.77 min;

### Synthesis of (R)-2-methyl-N—((S)-1-(4-(4-methylthiazol-5-yl)phenyl) hex-5-en-1-yl) propane-2-sulfinamide (Intermediate 8)

To a solution of (R,Z)-2-methyl-N-(1-(4-(4-methylthiazol-5-yl)phenyl) hex-5-en-1-ylidene) propane-2-sulfinamide (9.00 g, 1 Eq, 24.0 mmol) in THF (100 mL) was added Lithium Tri-sec-butylborohydride (L-selectride), 1M in THF (5.94 g, 6.7 mL, 1.3 Eq, 31.2 mmol) at −70° C. The mixture was stirred at −70° C. for 1 hour. LCMS indicated completion of reaction. The reaction was poured into water (50.0 mL) and extracted with EA (100.0 mL), then washed with brine (50.0 mL) and dried over sodium sulfate, filtered and concentrated under vacuum. The residue was purified by silica gel chromatography (100-200 mesh silica gel, 50% of EtOAc in PE) to afford (R)-2-methyl-N—((S)-1-(4-(4-methylthiazol-5-yl)phenyl) hex-5-en-1-yl) propane-2-sulfinamide (7.80 g, 17 mmol, 69%, 80% Purity) as a yellow solid. LC purity (0.1% TFA): 92.2% (UV at 214 nm)/MS: 377.2 [M+H]; Retention time: 1.60 min.

### Synthesis of (R)—N—((S)-6-hydroxy-1-(4-(4-methylthiazol-5-yl)phenyl) hexyl)-2-methylpropane-2-sulfinamide (Intermediate 9)

To a solution of (R)-2-methyl-N—((S)-1-(4-(4-methylthiazol-5-yl)phenyl) hex-5-en-1-yl) propane-2-sulfinamide (5.00 g, 1 eq, 13.3 mmol) in THF (80.0 mL) was added 9-Borabicyclo[3.3.1]nonane Solution (6.48 g, 7.60 mL, 4 Eq, 53.1 mmol). The mixture was stirred at 0° C. for 2 hours. H₂O₂ (4.52 g, 4.07 mL, 10 Eq, 133 mmol), H₂O (20.0 mL) and NaOH (5.31 g, 10 Eq, 133 mmol) were added to the mixture under N₂. The mixture was stirred at 0° C. for 4 hours.

LCMS indicated completion of reaction. The reaction was poured into water (50.0 mL) and extracted with EA (100.0 mL), then washed with brine (50.0 mL) and dried over sodium sulfate, filtered and concentrated under vacuum. The residue was purified by silica gel chromatography (100-200 mesh silica gel, 30% of EtOAc in PE) to afford (R)—N—((S)-6-hydroxy-1-(4-(4-methylthiazol-5-yl)phenyl) hexyl)-2-methylpropane-2-sulfinamide (2.80 g, 6.7 mmol, 51%, 95% Purity) as a yellow oil. LC purity (0.1% TFA): 85.3% (UV at 254 nm)/MS: 395.2 [M+H]; Retention time: 1.413 min.

### Synthesis of(S)-6-amino-6-(4-(4-methylthiazol-5-yl)phenyl) hexan-1-ol (Intermediate 10)

To a solution of (R)—N—((S)-6-hydroxy-1-(4-(4-methylthiazol-5-yl)phenyl) hexyl)-2-methylpropane-2-sulfinamide (2.80 g, 1 Eq, 7.10 mmol) in DCM (20.0 mL) was added HCl (2.59 g, 10 Eq, 71.0 mmol), 1,2-Dioxane (6.25 g, 10 Eq, 71.0 mmol). The mixture was stirred at 25° C. for 2 hours. LCMS indicated completion of reaction. The mixture was concentrated under vacuum to obtain(S)-6-amino-6-(4-(4-methylthiazol-5-yl)phenyl) hexan-1-ol (2.20 g, 7.0 mmol, 98%, 92% Purity) as a yellow oil. LC purity (0.1% TFA): 95.99% (UV at 254 nm)/MS: 291.2 [M+H]; Retention time: 1.03 min.

### Synthesis of(S)-6-amino-6-(4-(4-methylthiazol-5-yl)phenyl) hexan-1-ol (Intermediate 12)

To a solution of(S)-6-amino-6-(4-(4-methylthiazol-5-yl)phenyl) hexan-1-ol (900 mg, 1 Eq, 3.10 mmol) in DMF (10.0 mL) were added TEA (941 mg, 1.30 mL, 3 Eq, 9.30 mmol), HATU (1.18 g, 1 Eq, 3.10 mmol), (2S,4R)-4-((tert-butyldimethylsilyl)oxy)-1-((S)-2-(1-fluorocyclopropane-1-carboxamido)-3,3-dimethylbutanoyl)pyrrolidine-2-carboxylic acid (1.52 g, 1.1 Eq, 3.41 mmol). The reaction was stirred at 25° C. for 2 hours. LCMS indicated completion of reaction. The reaction was poured into water (30.0 mL) and extracted with EA (50.0 mL), then washed with brine (30.0 mL) and dried over sodium sulfate, filtered and concentrated under vacuum. The residue was purified by silica gel chromatography (100-200 mesh silica gel, 50% of EtOAc in PE) to afford (2S,4R)-4-((tert-butyldimethylsilyl)oxy)-1-((S)-2-(1-fluorocyclopropane-1-carboxamido)-3,3-dimethylbutanoyl)-N—((S)-6-hydroxy-1-(4-(4-methylthiazol-5-yl)phenyl) hexyl)pyrrolidine-2-carboxamide (820 mg, 1.1 mmol, 35%, 95% Purity) as a blue solid. LC purity (0.1% TFA): 68.26% (UV at 254 nm)/MS: 717.1 [M+H]; Retention time: 1.79 min.

### Synthesis of (2S,4R)-4-((tert-butyldimethylsilyl)oxy)-1-((S)-2-(1-fluorocyclopropane-1-carboxamido)-3,3-dimethylbutanoyl)-N—((S)-1-(4-(4-methylthiazol-5-yl)phenyl)-6-oxohexyl)pyrrolidine-2-carboxamide (Intermediate 13)

To a solution of (2S,4R)-4-((tert-butyldimethylsilyl)oxy)-1-((S)-2-(1-fluorocyclopropane-1-carboxamido)-3,3-dimethylbutanoyl)-N—((S)-6-hydroxy-1-(4-(4-methylthiazol-5-yl)phenyl) hexyl)pyrrolidine-2-carboxamide (780 mg, 1 Eq, 1.09 mmol) in DCM (10.0 mL) was added Dess-Martin periodinane (692 mg, 507 μL, 1.5 Eq, 1.63 mmol). The mixture was stirred at 25° C. for 2 hours. LCMS indicated completion of reaction. The reaction was poured into water (20.0 mL) and extracted with EA (20.0 mL), then washed with brine (10.0 mL) and dried over sodium sulfate, filtered and concentrated under vacuum. The resulting residue was purified by silica gel chromatography (100-200 mesh silica gel, 50% of EtOAc in PE) to afford (2S,4R)-4-((tert-butyldimethylsilyl)oxy)-1-((S)-2-(1-fluorocyclopropane-1-carboxamido)-3,3-dimethylbutanoyl)-N—((S)-1-(4-(4-methylthiazol-5-yl)phenyl)-6-oxohexyl)pyrrolidine-2-carboxamide (480.0 mg, 0.64 mmol, 59%, 95% Purity) as a blue oil. LC purity (0.1% TFA): 78.21% (UV at 254 nm)/MS: 715.1 [M+H]; Retention time: 1.784 min.

### Synthesis of (2S,4R)—N—((S)-6-(4-(4-bromo-7,7-dimethyl-5-oxo-5,7-dihydroindolo[1,2-a]quinazol in-9-yl)piperidin-1-yl)-1-(4-(4-methylthiazol-5-yl)phenyl) hexyl)-4-((tert-butyldimethylsilyl)oxy)-1-((S)-2-(1-fluorocyclopropane-1-carboxamido)-3,3-dimethylbutanoyl)pyrrolidine-2-carboxamide (Intermediate 15)

To a solution of (2S,4R)-4-((tert-butyldimethylsilyl)oxy)-1-((S)-2-(1-fluorocyclopropane-1-carboxamido)-3,3-dimethylbutanoyl)-N—((S)-1-(4-(4-methylthiazol-5-yl)phenyl)-6-oxohexyl)pyrrolidine-2-carboxamide (50.0 mg, 1 Eq, 69.9 μmol) in MeOH (2.00 mL) was added 4-bromo-7,7-dimethyl-9-(piperidin-4-yl)indolo[1,2-a]quinazolin-5 (7H)-one (35.6 mg, 1.2 Eq, 83.9 μmol), and Sodium cyanoborohydride (17.6 mg, 16.5 L, 4 Eq, 280 μmol) under N₂. The mixture was stirred at 25° C. for 3 hours. LCMS indicated completion of reaction. The mixture was filtered and concentrated under vacuum. The resulting residue was purified by silica gel chromatography (100-200 mesh silica gel, 5% of MeOH in DCM) to afford (2S,4R)—N—((S)-6-(4-(4-bromo-7,7-dimethyl-5-oxo-5,7-dihydroindolo[1,2-a]quinazolin-9-yl)piperidin-1-yl)-1-(4-(4-methylthiazol-5-yl)phenyl) hexyl)-4-((tert-butyldimethylsilyl)oxy)-1-((S)-2-(1-fluorocyclopropane-1-carboxamido)-3,3-dimethylbutanoyl)pyrrolidine-2-carboxamide (35.0 mg, 30 μmol, 42%, 95% Purity) as a yellow solid. LC purity (0.1% FA): 91.97% at 254 nm/MS: 562.8 Retention time: 1.65 min.

### Synthesis of (2S,4R)—N—((S)-6-(4-(4-bromo-7,7-dimethyl-5-oxo-5,7-dihydroindolo[1,2-a]quinazol in-9-yl)piperidin-1-yl)-1-(4-(4-methylthiazol-5-yl)phenyl) hexyl)-1-((S)-2-(1-fluorocyclopropane-1-carboxamido)-3,3-dimethylbutanoyl)-4-hydroxypyrrolidine-2-carboxamide (compound B2)

To a solution of (2S,4R)—N—((S)-6-(4-(4-bromo-7,7-dimethyl-5-oxo-5,7-dihydroindolo[1,2-a]quinazolin-9-yl)piperidin-1-yl)-1-(4-(4-methylthiazol-5-yl)phenyl) hexyl)-4-((tert-butyldimethylsilyl)oxy)-1-((S)-2-(1-fluorocyclopropane-1-carboxamido)-3,3-dimethylbutanoyl)pyrrolidine-2-carboxamide (30.0 mg, 1 Eq, 26.7 μmol) in DCM (1.00 mL) was added TFA (2.96 g, 2.00 mL). The reaction was stirred at 25° C. for 2 hours. LCMS indicated completion of reaction. The reaction was quenched with methanol, concentrated under vacuum. The resulting residue was purified by prep-HPLC, eluted MeCN in H₂O from 10% to 85% (0.1% TFA) to afford (2S,4R)—N—((S)-6-(4-(4-bromo-7,7-dimethyl-5-oxo-5,7-dihydroindolo[1,2-a]quinazolin-9-yl)piperidin-1-yl)-1-(4-(4-methylthiazol-5-yl)phenyl) hexyl)-1-((S)-2-(1-fluorocyclopropane-1-carboxamido)-3,3-dimethylbutanoyl)-4-hydroxypyrrolidine-2-carboxamide Compound B2 (5.01 mg, 4.91 μmol, 18.4%, 98.84% purity) as a white solid. LC purity (0.1% FA): 94.77% 254 nm/MS: 505.8 Retention time: 1.358 min. ¹H NMR (400 MHz, MeOD) δ 8.88 (s, 1H), 8.46 (d, J=8.6 Hz, 1H), 8.11 (d, J=8.4 Hz, 1H), 7.91 (d, J=7.4 Hz, 1H), 7.75 (d, J=8.4 Hz, 1H), 7.56 (s, 1H), 7.49-7.44 (m, 5H), 4.95-4.92 (m, 1H), 4.77-4.76 (m, 1H), 4.62-4.60 (m, 1H), 4.47-4.45 (m, 1H), 3.83-3.81 (m, 2H), 3.67-3.65 (m, 2H), 3.13-3.02 (m, 5H), 2.49 (s, 3H), 2.22-2.18 (m, 3H), 2.02-1.87 m, 7H), 1.62-1.61 (m, 7H), 1.51-1.50 (m, 3H), 1.40-1.35 (m, 4H), 1.07 (s, 9H).

Compound B3. (2S,4R)—N—((S)-6-(4-(4-bromo-7,7-dimethyl-5-oxo-5,7-dihydroindolo[1,2-a]quinazolin-9-yl)piperidin-1-yl)-1-(4-(4-methylthiazol-5-yl)phenyl) hexyl)-1-((S)-2-(1-cyanocyclopropane-1-carboxamido)-3,3-dimethylbutanoyl)-4-hydroxypyrrolidine-2-carboxamide

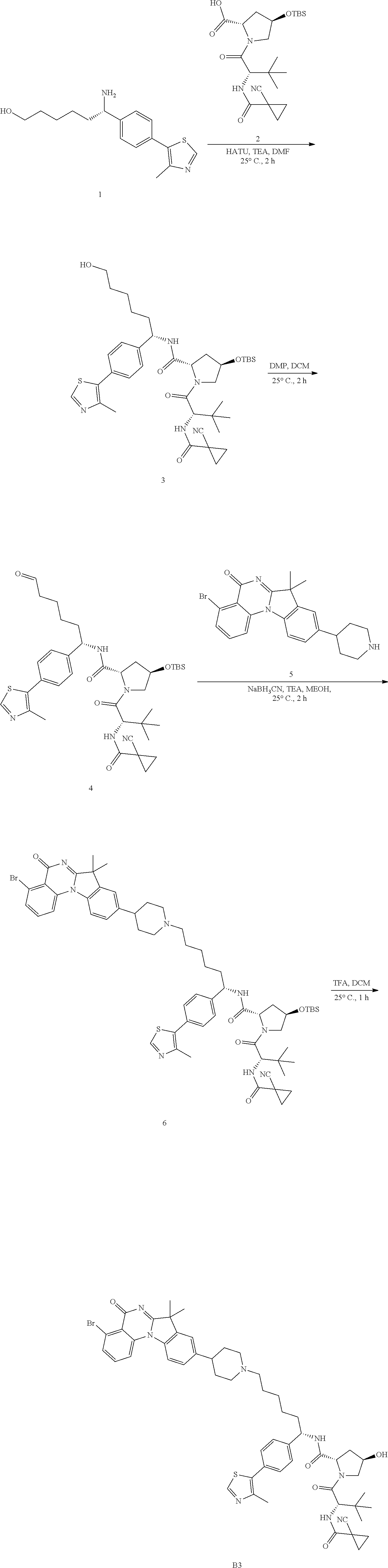

### Synthesis of (2S,4R)-4-((tert-butyldimethylsilyl)oxy)-1-((S)-2-(1-cyanocyclopropane-1-carboxamido)-3,3-dimethylbutanoyl)-N—((S)-6-hydroxy-1-(4-(4-methylthiazol-5-yl)phenyl) hexyl)pyrrolidine-2-carboxamide (Intermediate 3)

To a solution of (2S,4R)-4-((tert-butyldimethylsilyl)oxy)-1-((S)-2-(1-cyanocyclopropane-1-carboxamido)-3,3-dimethylbutanoyl)pyrrolidine-2-carboxylic acid (311 mg, 1 Eq, 689 μmol) in DMF (2.00 mL) was added HATU (340 mg, 1.3 eq, 895 μmol), DIEA (356 mg, 480 μL, 4 eq, 2.75 mmol). The mixture was stirred at 25° C. for 2 hours. LCMS indicated completion of reaction. The reaction mixture was poured into water (50 mL), extracted with EA (40 mL×3). The combined organic layers were washed with brine (30.0 mL×3), dried over sodium sulfate, filtered and concentrated under vacuum. The resulting residue was purified by flash chromatography, eluted with 20% EA in PE to afford (2S,4R)-4-((tert-butyldimethylsilyl)oxy)-1-((S)-2-(1-cyanocyclopropane-1-carboxamido)-3,3-dimethylbutanoyl)-N—((S)-6-hydroxy-1-(4-(4-methylthiazol-5-yl)phenyl) hexyl)pyrrolidine-2-carboxamide (250 mg, 0.33 mmol, 48% yield) as a yellow solid. LC purity (0.03% TFA): 99.54% (UV at 254 nm)/MS: 724.2 [M+H]; Retention time: 1.09 min

### Synthesis of (2S,4R)-4-((tert-butyldimethylsilyl)oxy)-1-((S)-2-(1-cyanocyclopropane-1-carboxamido)-3,3-dimethylbutanoyl)-N—((S)-6-hydroxy-1-(4-(4-methylthiazol-5-yl)phenyl) hexyl)pyrrolidine-2-carboxamide (Intermediate 4)

To a solution of (2S,4R)-4-((tert-butyldimethylsilyl)oxy)-1-((S)-2-(1-cyanocyclopropane-1-carboxamido)-3,3-dimethylbutanoyl)-N—((S)-6-hydroxy-1-(4-(4-methylthiazol-5-yl)phenyl) hexyl)pyrrolidine-2-carboxamide (30.0 mg, 1 eq, 41.4 μmol) in DCM (1.00 mL) was added DMP (26.4 mg, 1.5 eq, 62.2 μmol). The mixture was stirred at 25° C. for 2 hours. LCMS indicated completion of reaction. The mixture was filtered and concentrated under vacuum to afford (2S,4R)-4-((tert-butyldimethylsilyl)oxy)-1-((S)-2-(1-cyanocyclopropane-1-carboxamido)-3,3-dimethylbutanoyl)-N—((S)-1-(4-(4-methylthiazol-5-yl)phenyl)-6-oxohexyl)pyrrolidine-2-carboxamide (30 mg, 0.31 mmol, 75% yield) as a yellow solid. LC purity (0.1% FA): 75.68% (UV at 254 nm)/MS: 722.4 [M+H]; Retention time: 1.86 min.

### Synthesis of (2S,4R)—N—((S)-6-(4-(4-bromo-7,7-dimethyl-5-oxo-5,7-dihydroindolo[1,2-a]quinazol in-9-yl)piperidin-1-yl)-1-(4-(4-methylthiazol-5-yl)phenyl) hexyl)-4-((tert-butyldimethylsilyl)oxy)-1-((S)-2-(1-cyanocyclopropane-1-carboxamido)-3,3-dimethylbutanoyl)pyrrolidine-2-carboxamide (Intermediate 6)

To a solution of (2S,4R)-4-((tert-butyldimethylsilyl)oxy)-1-((S)-2-(1-cyanocyclopropane-1-carboxamido)-3,3-dimethylbutanoyl)-N—((S)-1-(4-(4-methylthiazol-5-yl)phenyl)-6-oxohexyl)pyrrolidine-2-carboxamide (30.0 mg, 1 eq, 41.5 μmol) in MeOH (0.50 mL) was added 4-bromo-7,7-dimethyl-9-(piperidin-4-yl)indolo[1,2-a]quinazolin-5 (7H)-one (21.2 mg, 1.2 eq, 49.9 μmol), and TEA (12.6 mg, 17.4 μL, 3 eq, 125 μmol). The mixture was stirred for 30 min, Sodium cyanoborohydride (7.83 mg, 7.35 L, 3 eq, 125 μmol) was added, the mixture was stirred at 25° C. for 2 hours. LCMS indicated completion of reaction. The reaction was concentrated under vacuum and purified by flash chromatography, eluted with 15% MeOH in

DCM to afford (2S,4R)—N—((S)-6-(4-(4-bromo-7,7-dimethyl-5-oxo-5,7-dihydroindolo[1,2-a]quinazolin-9-yl)piperidin-1-yl)-1-(4-(4-methylthiazol-5-yl)phenyl) hexyl)-4-((tert-butyldimethylsilyl)oxy)-1-((S)-2-(1-cyanocyclopropane-1-carboxamido)-3,3-dimethylbutanoyl)pyrrolidine-2-carboxamide (40.0 mg, 35.4 μmol, 68.08% yield) as a yellow solid. LC purity (0.1% FA): 80.31% (UV at 254 nm)/MS: 565.4 [M/2+H]; Retention time: 1.57

### Synthesis of (2S,4R)—N—((S)-6-(4-(4-bromo-7,7-dimethyl-5-oxo-5,7-dihydroindolo[1,2-a]quinazolin-9-yl)piperidin-1-yl)-1-(4-(4-methylthiazol-5-yl)phenyl) hexyl)-1-((S)-2-(1-cyanocyclopropane-1-carboxamido)-3,3-dimethylbutanoyl)-4-hydroxypyrrolidine-2-carboxamide (Compound B3)

To a solution of (2S,4R)—N—((S)-6-(4-(4-bromo-7,7-dimethyl-5-oxo-5,7-dihydroindolo[1,2-a]quinazolin-9-yl)piperidin-1-yl)-1-(4-(4-methylthiazol-5-yl)phenyl) hexyl)-4-((tert-butyldimethylsilyl)oxy)-1-((S)-2-(1-cyanocyclopropane-1-carboxamido)-3,3-dimethylbutanoyl)pyrrolidine-2-carboxamide (30.0 mg, 1 eq, 26.5 μmol) in DCM (2.00 mL) was added TFA (0.30 mL, 3.9 mmol), and the reaction was stirred at 25° C. for 1 hour. LCMS indicated completion of reaction. The reaction was concentrated under vacuum and purified by prep-HPLC, eluted with CH₃CN in H₂O (0.03% NH₄CO₃) from 5.0% to 95.0% (0.1% FA) to afford (2S,4R)—N—((S)-6-(4-(4-bromo-7,7-di methyl-5-oxo-5,7-dihydroindolo[1,2-a]quinazolin-9-yl)piperidin-1-yl)-1-(4-(4-methylthiazol-5-yl)phenyl)hexyl)-1-((S)-2-(1-cyanocyclopropane-1-carboxamido)-3,3-dimethylbutanoyl)-4-hydroxypyrrolidine-2-carboxamide Compound B3 (5.02 mg, 4.78 μmol, 18.0% yield, 96.8% purity) as a white solid. LC purity (0.1% FA): 98.38% (UV at 254 nm)/MS: 508.4 [M/2+1]; Retention time: 1.36 min. ¹H NMR (400 MHz, MeOD) δ 8.91 (s, 1H), 8.47 (d, J=8.1 Hz, 1H), 8.12 (d, J=8.6 Hz, 1H), 7.93 (d, J=7.8 Hz, 1H), 7.79 (t, J=8.3 Hz, 1H), 7.60 (d, J=1.7 Hz, 1H), 7.52-7.43 (m, 5H), 4.96-4.93 (m, 1H), 4.69-4.58 (m, 2H), 4.50-4.48 (m, 1H), 3.86-3.83 (m, 1H), 3.78-3.67 (m, 3H), 3.23-3.00 (m, 5H), 2.50 (s, 3H), 2.28-2.15 (m, 3H), 2.14-2.01 (m, 2H), 1.98-1.85 (m, 5H), 1.70-1.64 (m, 11H), 1.58-1.51 (m, 3H), 1.09 (s, 9H).

Compound B4. (2S,4R)-1-((R)-2-(3-(2-(4-(4-chloro-7,7-dimethyl-5-oxo-5,7-dihydroindolo[1,2-a]quinazolin-9-yl)piperidin-1-yl)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide

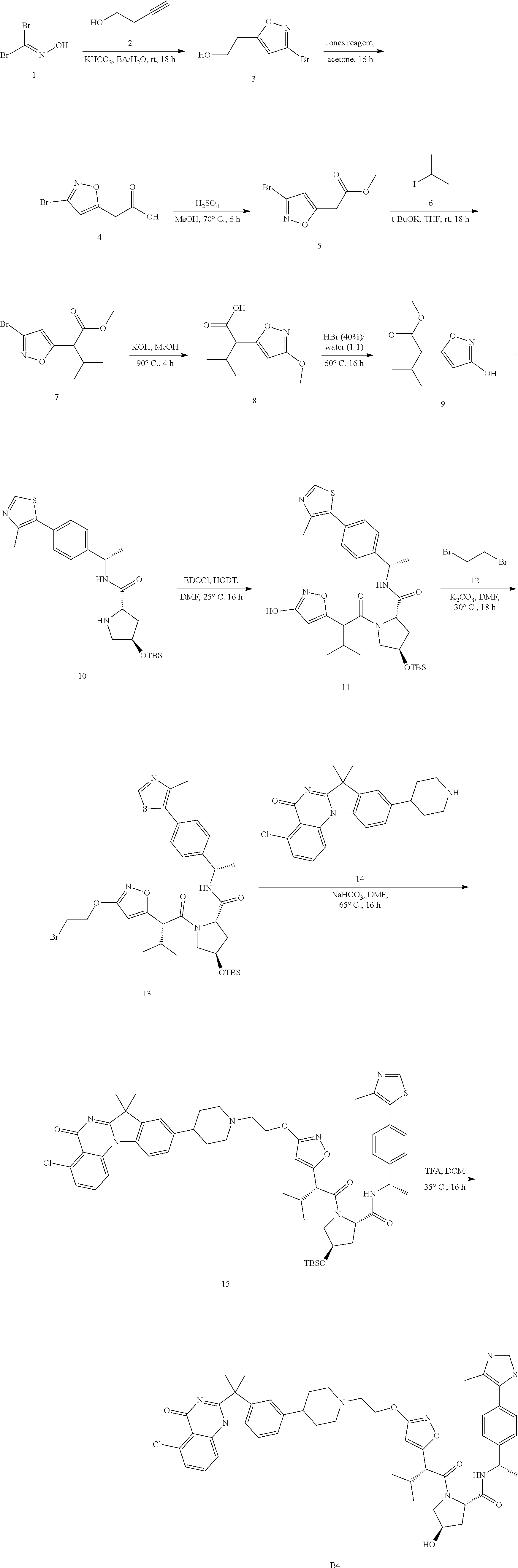

### Synthesis of 2-(3-bromoisoxazol-5-yl) ethan-1-ol (Intermediate 3)

To a solution of but-3-yn-1-ol (27.6 g, 4 eq, 394 mmol) and Potassium bicarbonate (29.6 g, 3 eq, 296 mmol) in EA (200 mL) and Water (20.0 mL) was added hydroxycarbonimidic dibromide (20.0 g, 1 eq, 98.6 mmol) in EA (50 mL). The mixture was stirred at room temperature for 16 hours. LCMS indicated completion of reaction. Water (100 mL) was added, extracted with EA (200 mL×3), the combined organic layers were washed with brine (100 mL), dried over anhydrous Na₂SO₄, filtered and concentrated under vacuum to give 2-(3-bromoisoxazol-5-yl) ethan-1-ol (32.0 g, 83.0 mmol, 85%, 95% Purity) as a yellow oil. LC purity (0.1% FA): 95% (UV at 214 nm)/MS: 192.0 [M+H]; Retention time: 0.59 min.

### Synthesis of 2-(3-bromoisoxazol-5-yl) acetic acid (Intermediate 4)

To a solution of 2-(3-bromoisoxazol-5-yl) ethan-1-ol (18.0 g, 1 eq, 93.7 mmol) in Acetone (250 mL) was added Jones reagent (46.9 g, 187 mL, 2.50 molar, 5 eq, 469 mmol) dropwise. The reaction was stirred at room temperature for 16 hours. LCMS indicated completion of reaction.

Water (100 mL) was added, extracted with FA (200 mL×3), the combined organic layers were washed with brine (100 mL), dried over anhydrous Na₂SO₄, filtered and concentrated under vacuum to give 2-(3-bromoisoxazol-5-yl) acetic acid (17.0 g, 66 mmol, 52% Purity) as a yellow oil. LC purity (0.1% FA): 52% (UV at 214 nm)/MS: 206.0 [M+H]; Retention time: 0.75 min.

### Synthesis of methyl 2-(3-bromoisoxazol-5-yl)acetate (Intermediate 5)

To a solution of 2-(3-bromoisoxazol-5-yl) acetic acid (17.0 g, 1 eq, 82.5 mmol) in MeOH (250 mL) was added sulfuric acid (80.9 g, 10 eq, 825 mmol) dropwise. The reaction was stirred at 70° C. for 6 hours. LCMS indicated completion of reaction. Water (100 mL) was added, extracted with EA (200 mL×3), the combined organic layers were washed with brine (100 mL), dried over anhydrous Na₂SO₄, filtered and concentrated under vacuum to get give methyl 2-(3-bromoisoxazol-5-yl)acetate (16.0 g, 72.7 mmol, 88.1%) as a green oil. LC purity (0.1% FA): 42% (UV at 214 nm)/MS: 220.0 [M+H]; Retention time: 1.19 min.

### Synthesis of methyl 2-(3-bromoisoxazol-5-yl)-3-methylbutanoate (Intermediate 7)

To a solution of methyl 2-(3-bromoisoxazol-5-yl)acetate (16.0 g, 1 eq, 72.7 mmol) and 2-methylpropan-2-olate potassium (12.2 g, 13.7 mL, 1.5 eq, 109 mmol) in THF (200 mL) was added Isopropyl iodide (18.5 g, 10.9 mL, 1.5 eq, 109 mmol) dropwise at 0° C. under N₂. The solution was stirred at room temperature for 16 hours. LCMS indicated completion of reaction. Water (300 mL) was added, extracted with EA (200 mL×2), the combined organic layers were washer with brine (100 mL), dried over anhydrous Na₂SO₄, filtered and concentrated under vacuum to give the methyl 2-(3-bromoisoxazol-5-yl)-3-methylbutanoate (17.0 g, 64.9 mmol, 89.2%) as a yellow oil. LC purity (0.1% FA): 30% (UV at 214 nm)/MS: 262.0 [M+H]; Retention time: 1.59 min.

### Synthesis of 2-(3-methoxyisoxazol-5-yl)-3-methylbutanoic acid (Intermediate 8)

To a solution of methyl 2-(3-bromoisoxazol-5-yl)-3-methylbutanoate (17.0 g, 1 eq, 64.9 mmol) in MeOH (40.0 mL) was added Potassium hydroxide (36.4 g, 37.8 mL, 10 eq, 649 mmol) at 0° C. The reaction was stirred at 90° C. for 16 hours. LCMS indicated completion of reaction. Water (300 mL) was added, adjusted PH to 6 by 1N HCl, extracted with EA (200 mL×2), the combined organic layers were washed with brine (100 mL), dried over anhydrous Na₂SO₄, filtered and concentrated under vacuum to give 2-(3-methoxyisoxazol-5-yl)-3-methylbutanoic acid (7.00 g, 40.2 mmol, 54.1%) as a yellow oil. LC purity (0.1% FA): 88% (UV at 214 nm)/MS: 200.0 [M+H]; Retention time: 0.20 min.

### Synthesis of 2-(3-hydroxyisoxazol-5-yl)-3-methylbutanoic acid (Intermediate 9)

To a solution of 2-(3-methoxyisoxazol-5-yl)-3-methylbutanoic acid (7.00 g, 1 eq, 35.1 mmol) in Acetic Acid (30.0 mL) was added Hydrogen bromide in water (30.0 mL, 48% Wt, 7.48 eq, 263 mmol) at 0° C. The reaction was stirred at 60° C. for 16 hours. LCMS indicated completion of reaction. The resulting residue was purified by Combi-flash, eluted with MeCN in H₂O from 10% to 90% (0.1% TFA) to give 2-(3-hydroxyisoxazol-5-yl)-3-methylbutanoic acid (3.60 g, 19.4 mmol, 55.3%) as a yellow oil. LC purity (0.1% FA): 90% (UV at 214 nm)/MS: 186.0 [M+H]; Retention time: 1.02 min.

### Synthesis of (2S,4R)-4-((tert-butyldimethylsilyl)oxy)-1-(2-(3-hydroxyisoxazol-5-yl)-3-methylbutanoyl)-N—((S)-1-(4-(4-methylthiazol-5-yl)phenyl)ethyl)pyrrolidine-2-carboxamide (Intermediate 11)

To a solution of 2-(3-hydroxyisoxazol-5-yl)-3-methylbutanoic acid (300 mg, 1 eq, 1.62 mmol) in DMF (14.0 mL) was added 1-(3-Dimethylaminopropyl)-3-ethylcarbodiimideHydrochloride (EDCl) (404 mg, 1.3 eq, 2.11 mmol), N-ethyl-N-isopropylpropan-2-amine (1.05 g, 5 eq, 8.10 mmol), 1H-benzo[d][1,2,3]triazol-1-ol hydrate (323 mg, 1.3 eq, 2.11 mmol) and (2S,4R)-4-((tert-butyldimethylsilyl)oxy)-N—((S)-1-(4-(4-methylthiazol-5-yl)phenyl)ethyl)pyrrolidine-2-carboxamide (722 mg, 1 eq, 1.62 mmol). The mixture was stirred at 25° C. for 16 hours. LCMS indicated completion of reaction. The reaction was quenched by water, extracted with EA (50 mL×3), washed with brine (50 mL), dried over sodium sulfate, filtered and concentrate dunder vacuum. The resulting residue was purified by silica gel column chromatography (100-200 mesh silica gel, 10% of MeOH in DCM) to get (2S,4R)-4-((tert-butyldimethylsilyl)oxy)-1-(2-(3-hydroxyisoxazol-5-yl)-3-methylbutanoyl)-N—((S)-1-(4-(4-methylthiazol-5-yl)phenyl)ethyl)pyrrolidine-2-carboxamide (230 mg, 375 μmol, 23.2%) as a white solid. LC purity (0.1% FA): 70% (UV at 254 nm)/MS: 613.3 [M+H]; Retention time: 1.70 min.

### Synthesis of (2S,4R)-1-((R)-2-(3-(2-bromoethoxy)isoxazol-5-yl)-3-methylbutanoyl)-4-((tert-butyldimethylsilyl)oxy)-N—((S)-1-(4-(4-methylthiazol-5-yl)phenyl)ethyl)pyrrolidine-2-carboxamide (Intermediate 13)

To a solution of ((2S,4R)-4-((tert-butyldimethylsilyl)oxy)-1-(2-(3-hydroxyisoxazol-5-yl)-3-methylbutanoyl)-N—((S)-1-(4-(4-methylthiazol-5-yl)phenyl)ethyl)pyrrolidine-2-carboxamide (455.0 mg, 1 eq, 0.74 mmol) and 1,2-Dibromoethane (1.39 g, 10 eq, 7.42 mmol) in DMF (3.0 mL) were added and K₂CO₃ (308.0 mg, 3 eq, 2.23 mmol) at 25° C., the mixture solution was stirred at 25° C. for 16 hours. The reaction was extracted with EtOAc (20.0 mL×3) and the combined organic layers were washed with water (20.0 mL×3) and washed with brine (20.0 mL) dried over sodium sulfate, filtered and concentrated under vacuum. The resulting residue was purified by prep-HPLC, eluted with MeCN in H₂O from 50% to 90%, (0.1% of FA) to give (2S,4R)-1-((R)-2-(3-(2-bromoethoxy)isoxazol-5-yl)-3-methylbutanoyl)-4-((tert-butyldimethylsilyl)oxy)-N—((S)-1-(4-(4-methylthiazol-5-yl)phenyl)ethyl)pyrrolidine-2-carboxamide (150.0 mg, 0.21 mmol, 28.1% yield) as a white solid. LC purity (0.1% FA): 98% (UV at 254 nm)/MS: 719.0 [M+H]; Retention time: 1.97 min.

### Synthesis of (2S,4R)-4-((tert-butyldimethylsilyl)oxy)-1-((R)-2-(3-(2-(4-(4-chloro-7,7-dimethyl-5-oxo-5,7-dihydroindolo[1,2-a]quinazolin-9-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide (Intermediate 15)

To a solution of 4-chloro-7,7-dimethyl-9-(piperidin-4-yl)indolo[1,2-a]quinazolin-5 (7H)-o ne (42.2 mg, 2 eq, 111 μmol) in DMF (5.0 mL) was added sodium hydrogen carbonate (70.0 mg, 15 eq, 834 μmol) and (2S,4R)-1-((R)-2-(3-(2-bromoethoxy)isoxazol-5-yl)-3-methylbutanoyl)-4-((tert-butyldimethylsilyl)oxy)-N—((S)-1-(4-(4-methylthiazol-5-yl)phenyl)ethyl)pyrrolidine-2-carb oxamide (40.0 mg, 1 eq, 55.6 μmol). The mixture was stirred at 65° C. for 16 hours. LCMS indica ted completion of reaction. Water (30.0 mL) was added, extracted with EA (20.0 mL×2), the organic layers were washer with brine (20.0 mL), dried over anhydrous Na₂SO₄, filtered and concentrated under vacuum to get (2S,4R)-4-((tert-butyldimethylsilyl)oxy)-1-((R)-2-(3-(2-(4-(4-chloro-7,7-dimethyl-5-oxo-5,7-dihydroindolo[1,2-a]quinazolin-9-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide (40.0 mg, 39.3 μmol, 35.4%) as a white solid. LC purity (0.1% TFA): 63% (UV at 254 nm)/MS: 101 8.0 [M+H]; Retention time: 1.57 min.

### Synthesis of (2S,4R)-1-((R)-2-(3-(2-(4-(4-chloro-7,7-dimethyl-5-oxo-5,7-dihydroindolo[1,2-a]quinazolin-9-yl)piperidin-1-yl)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide (Compound B4)

To a solution of (2S,4R)-4-((tert-butyldimethylsilyl)oxy)-1-((R)-2-(3-(2-(4-(4-chloro-7,7-dimethyl-5-oxo-5,7-dihydroindolo[1,2-a]quinazolin-9-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide (40.0 mg, 1 Eq, 39.3 μmol) in DCM (1.00 mL) was added 2,2,2-trifluoroacetic acid (4.48 mg, 1.00 m L, 1 Eq, 39.3 μmol) at room temperature. The reaction was stirred 35° C. for 2 hours. LCMS indicated completion of reaction. The mixture was added DCM (20 mL) and concentrated under vacuum, repeated three times. The residue was purified by prep-HPLC, eluted with CH₃CN in water (0.1% FA) to afford (2S,4R)-1-((R)-2-(3-(2-(4-(4-chloro-7,7-dimethyl-5-oxo-5,7-dihydroindolo[1,2-a]quinazolin-9-yl)piperidin-1-yl)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide (22.45 mg, 24.57 μmol, 62.6% yield, 99.42% purity) as a white solid. LC purity (0.03% NH₃H₂O): 99.42% (UV at 254 nm)/M S: 904.4 [M+H]; Retention time: 1.36 min. ¹H NMR (400 MHz, MeOD) δ 8.40 (m, 1H), 8.08 (m, 1H), 7.83 (m, 1H), 7.65 (m, 1H), 7.56 (s, 1H), 7.48-7.33 (m, 5H), 6.05 (m, 1H), 5.02 (m, 1H), 4.80-4.36 (m, 5H), 3.85 (m, 1H), 3.67 (m, 2H), 3.51-3.42 (m, 2H), 3.26 (s, 2H), 2.95-2.73 (m, 3H), 2.45 (s, 3H), 2.24-2.13 (m, 1H), 2.08-1.92 (m, 5H), 1.59 (s, 6H), 1.55-1.50 (m, 3H), 1.06 (d, J=6.4 Hz, 3H), 0.90 (d, J=6.8 Hz, 3H).

Compound B5. (2S,4R)-1-((S)-2-(2-(4-(4-bromo-7,7-dimethyl-5-oxo-5,7-dihydroindolo[1,2-a]quinazolin-9-yl)piperidin-1-yl) acetamido)-3,3-dimethylbutanoyl)-4-hydroxy-N—((S)-1-(4-(4-methylthiazol-5-yl)phenyl)ethyl)pyrrolidine-2-carboxamide

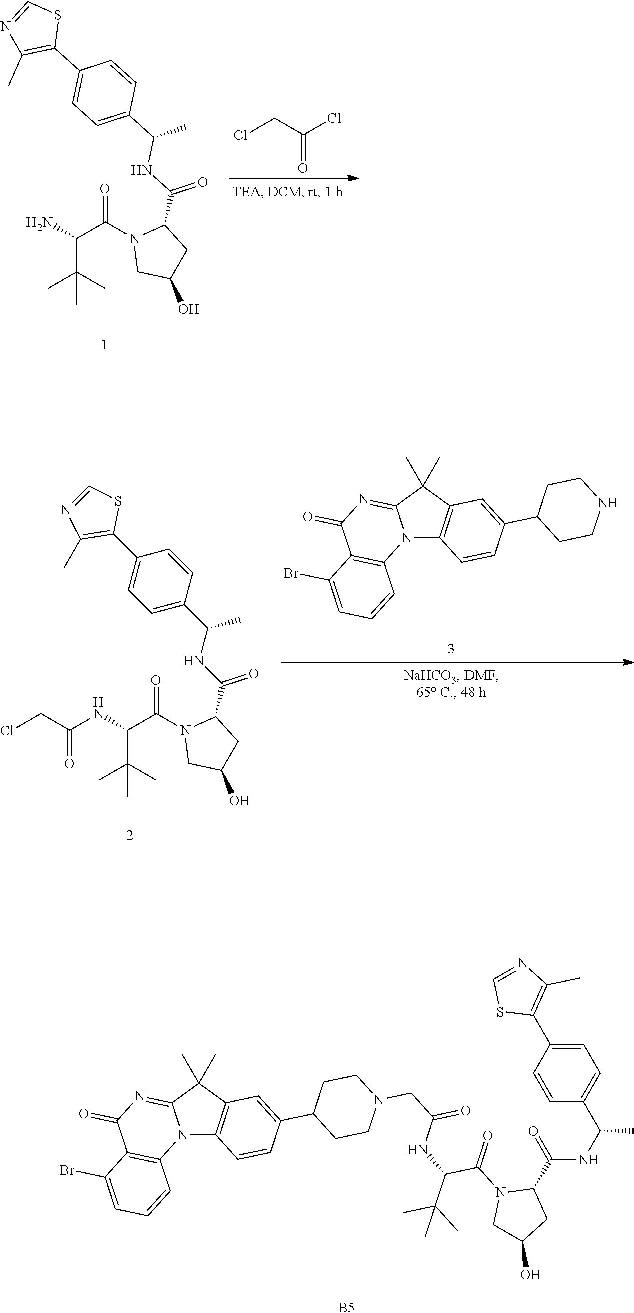 [SMILES: Cc1ncsc1-c1ccc([C@H](C)NC(=O)[C@@H]2C[C@@H](O)CN2C(=O)[C@@H](N)C(C)(C)C)cc1 ; O=C(Cl)CCl ; Cc1ncsc1-c1ccc([C@H](C)NC(=O)[C@@H]2C[C@@H](O)CN2C(=O)[C@@H](NC(=O)CCl)C(C)(C)C)cc1 ; CC1(C)c2cc(C3CCNCC3)ccc2-n2c1nc(=O)c1c(Br)cccc12 ; Cc1ncsc1-c1ccc([C@H](C)NC(=O)[C@@H]2C[C@@H](O)CN2C(=O)[C@@H](NC(=O)CN2CCC(c3ccc4c(c3)C(C)(C)c3nc(=O)c5c(Br)cccc5n3-4)CC2)C(C)(C)C)cc1 ; "B5"]

### Synthesis of 2 (2S,4R)-1-((S)-2-(2-chloroacetamido)-3,3-dimethylbutanoyl)-4-hydroxy-N—((S)-1-(4-(4-methylthiazol-5-yl)phenyl)ethyl)pyrrolidine-2-carboxamide (Intermediate 2)

To a solution of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (1.20 g, 1 eq, 2.70 mmol), TEA (546.0 mg, 0.75 mL, 2 eq, 5.40 mmol) in DCM (10.0 mL) was added 2-chloroacetyl chloride (366 mg, 1.2 Eq, 3.24 mmol). The mixture was then stirred at 25° C. for 1 hour. LCMS indicated completion of reaction. The mixture was poured into sat NH₄Cl (20 mL), extracted with EtOAc (20 mL×2). The combined organic layers were washed with brine (50.0 mL), dried over sodium sulfate, filtered and concentrated under vacuum. The resulting residue was purified by silica gel column chromatography (100-200 mesh silica gel, 30% of EtOAc in PE) to afford (2S,4R)-1-((S)-2-(2-chloroacetamido)-3,3-dimethylbutanoyl)-4-hydroxy-N—((S)-1-(4-(4-methylthiazol-5-yl)phenyl)ethyl)pyrrolidine-2-carboxamide (0.73 g, 1.4 mmol, 52%) as a yellow oil. LC purity (0.1% FA): 80.30% (UV at 254 nm)/MS: 521.1 [M+H]; Retention time: 1.104 min.

### Synthesis of (2S,4R)-1-((S)-2-(2-(4-(4-bromo-7,7-dimethyl-5-oxo-5,7-dihydroindolo[1,2-a]quinazolin-9-yl)piperidin-1-yl) acetamido)-3,3-dimethylbutanoyl)-4-hydroxy-N—((S)-1-(4-(4-methylthiazol-5-yl)phenyl)ethyl)pyrrolidine-2-carboxamide (Compound B6)

To a solution of (2S,4R)-1-((S)-2-(2-chloroacetamido)-3,3-dimethylbutanoyl)-4-hydroxy-N—((S)-1-(4-(4-methylthiazol-5-yl)phenyl)ethyl)pyrrolidine-2-carboxamide (80.0 mg, 1 eq, 0.15 mmol) in DMF (4.00 mL) was added NaHCO₃ (129.0 mg, 10 eq, 1.54 mmol), NaI (169.0 mg, 10 eq, 1.54 mmol), 4-bromo-7,7-dimethyl-9-(piperidin-4-yl)indolo[1,2-a]quinazolin-5 (7H)-one (78.2 mg, 1.2 eq, 0.18 mmol). The mixture was stirred at 65° C. for 16 hours. LCMS indicated completion of reaction. The mixture was purified by prep-HPLC, eluted with CH₃CN in H₂O (0.1 FA %) to afford (2S,4R)-1-((S)-2-(2-(4-(4-bromo-7,7-dimethyl-5-oxo-5,7-dihydroindolo[1,2-a]quinazolin-9-yl)piperidin-1-yl) acetamido)-3,3-dimethylbutanoyl)-4-hydroxy-N—((S)-1-(4-(4-methylthiazol-5-yl)phenyl)ethyl)pyrrolidine-2-carboxamide Compound B5 (60.0 mg, 0.065 mmol, 42.1%, 97.85% purity) as a white solid. LC purity (0.03% TFA): 100.00% (UV at 254 nm)/MS: 455.8 [M/2+H]; Retention time: 0.910 min. ¹H NMR (400 MHz, MeOD) δ 8.86 (s, 1H), 8.47 (d, J=8.4 Hz, 1H), 8.07-8.09 (m, 1H), 7.80-7.90 (m, 1H), 7.76-7.72 (m, 1H), 7.59 (s, 1H), 7.46-7.39 (m, 5H), 5.02 (d, J=7.2 Hz, 1H), 4.68 (s, 1H), 4.56-4.60 (m, 4H), 4.45 (s, 1H), 3.787 (d, J=10.8 Hz, 1H), 3.80-3.73 (m, 1H), 3.27-3.25 (m, 2H), 3.14-3.13 (s, 2H), 2.77-2.76 (m, 1H), 2.53 (s, 1H), 2.47-2.46 (m, 3H), 1.96-1.92 (m, 5H), 1.62 (s, 6H), 1.53-1.51 (m, 2H), 1.07 (s, 9H).

### Compound B6. (2S,4R)-1-((S)-2-(2-(4-(4-chloro-7,7-dimethyl-5-oxo-5,7-dihydroindolo[1,2-a]quinazolin-9-yl)piperidin-1-yl) acetamido)-3,3-dimethylbutanoyl)-4-hydroxy-N—((S)-1-(4-(4-methylthiazol-5-yl)phenyl)ethyl)pyrrolidine-2-carboxamide

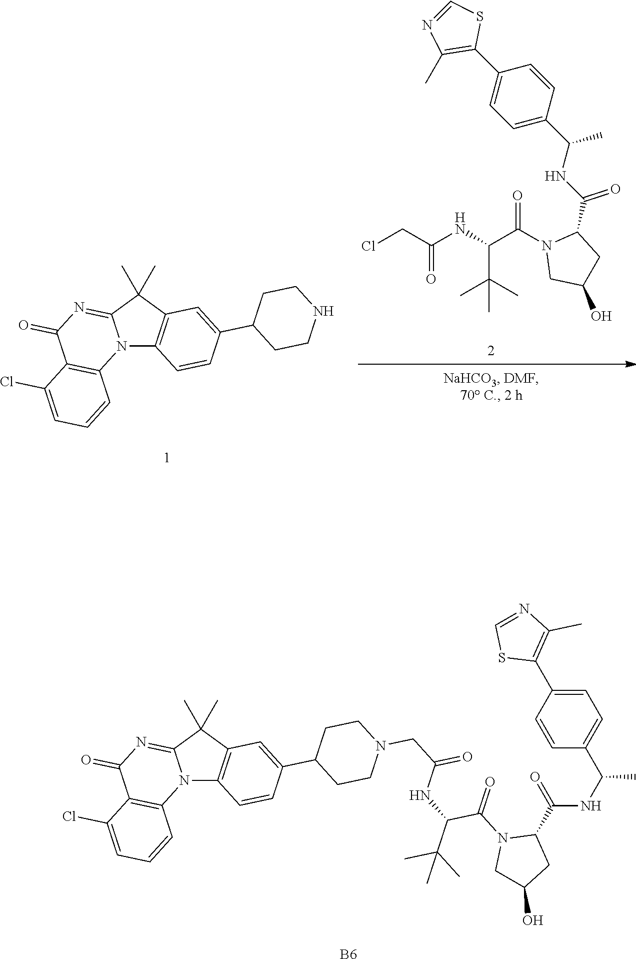 [SMILES: CC1(C)c2cc(C3CCNCC3)ccc2-n2c1nc(=O)c1c(Cl)cccc12 ; Cc1ncsc1-c1ccc([C@H](C)NC(=O)[C@@H]2C[C@@H](O)CN2C(=O)[C@@H](NC(=O)CCl)C(C)(C)C)cc1 ; "1" ; Cc1ncsc1-c1ccc([C@H](C)NC(=O)[C@@H]2C[C@@H](O)CN2C(=O)[C@@H](NC(=O)CN2CCC(c3ccc4c(c3)C(C)(C)c3nc(=O)c5c(Cl)cccc5n3-4)CC2)C(C)(C)C)cc1 ; "B6"]

### Synthesis of (2S,4R)-1-((S)-2-(2-(4-(4-chloro-7,7-dimethyl-5-oxo-5,7-dihydroindolo[1,2-a]quinazolin-9-yl)piperidin-1-yl) acetamido)-3,3-dimethylbutanoyl)-4-hydroxy-N—((S)-1-(4-(4-methylthiazol-5-yl)phenyl)ethyl)pyrrolidine-2-carboxamide (compound B6)

To a solution of 4-chloro-7,7-dimethyl-9-(piperidin-4-yl)indolo[1,2-a]quinazolin-5 (7H)-one (70.0 mg, 1 eq, 184 μmol) in DMF (0.30 mL) was added sodium bicarbonate (46.4 mg, 21.5 μL, 3 Eq, 553 μmol), (2S,4R)-1-((S)-2-(2-chloroacetamido)-3,3-dimethylbutanoyl)-4-hydroxy-N—((S)-1-(4-(4-methylthiazol-5-yl)phenyl)ethyl)pyrrolidine-2-carboxamide (57.6 mg, 0.6 eq, 111 μmol) under N₂, and the mixture was stirred at 70° C. for 2 hours. LCMS indicated completion of reaction. The reaction was poured into water (20 mL) and extracted with EA (20 mL), then washed brine (10 mL) and dried over sodium sulfate, filtered and concentrated under vacuum. The resulting residue was purified by prep-HPLC, eluted with CH₃CN in H₂O (0.1% TFA) to afford (2S,4R)-1-((S)-2-(2-(4-(4-chloro-7,7-dimethyl-5-oxo-5,7-dihydroindolo[1,2-a]quinazolin-9-yl)piperidin-1-yl) acetamido)-3,3-dimethylbutanoyl)-4-hydroxy-N—((S)-1-(4-(4-methylthiazol-5-yl)phenyl)ethyl)pyrrolidine-2-carboxamide Compound B6 (41.02 mg, 46.47 μmol, 25.2%, 97.94% purity) as a white solid. LC purity (0.1% FA): 93.5% (UV at 254 nm)/MS: 864.4 [M+H]; Retention time: 1.34 min; ¹H NMR (400 MHz, DMSO-d₆) δ 8.98 (s, 1H), 8.41 (t, J=9.0 Hz, 2H), 8.18-8.06 (m, 2H), 7.82 (t, J=8.3 Hz, 1H), 7.63 (d, J=7.9 Hz, 2H), 7.47-7.46 (m, 4H), 7.37 (d, J=8.2 Hz, 3H), 7.11 (d, J=7.8 Hz, 2H), 5.13 (d, J=3.0 Hz, 1H), 4.92 (d, J=7.3 Hz, 1H), 4.56 (d, J=9.8 Hz, 1H), 4.45 (t, J=8.4 Hz, 1H), 4.31-4.30 (m, 1H), 3.60-3.59 (m, 2H), 3.02-2.98 (m, 3H), 2.45 (s, 3H), 2.01-1.95 (m, 7H), 1.54-1.53 (s, 7H), 1.39 (d, J=7.0 Hz, 3H), 0.98-0.97 (s, 10H).

### Compound B7. (2S,4R)—N—((S)-6-(4-(4-chloro-7,7-dimethyl-5-oxo-5,7-dihydroindolo[1,2-a]quinazolin-9-yl)piperidin-1-yl)-1-(4-(4-methylthiazol-5-yl)phenyl) hexyl)-1-((S)-2-(1-fluorocyclopropane-1-carboxamido)-3,3-dimethylbutanoyl)-4-hydroxypyrrolidine-2-carboxamide

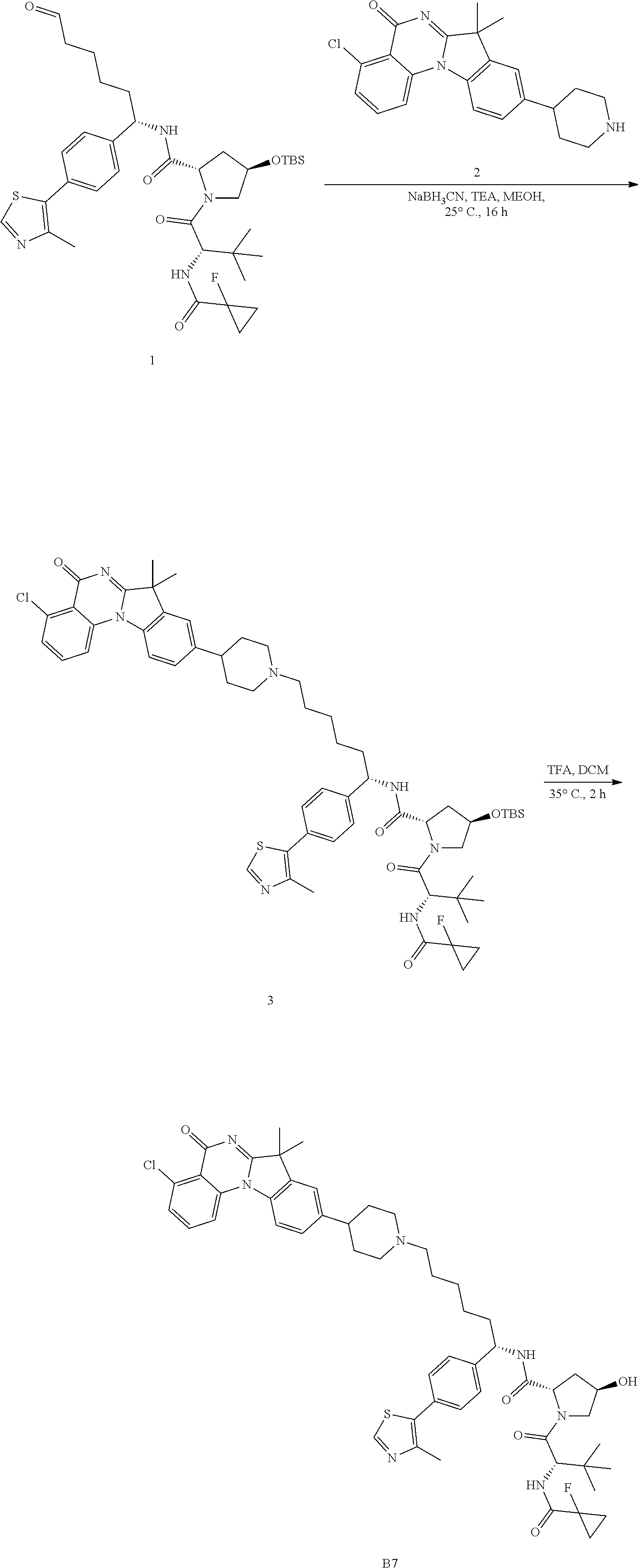

### Synthesis of ((2S,4R)-4-((tert-butyldimethylsilyl)oxy)-N—((S)-6-(4-(4-chloro-7,7-dimethyl-5-oxo-5,7-dihydroindolo[1,2-a]quinazolin-9-yl)piperidin-1-yl)-1-(4-(4-methylthiazol-5-yl)phenyl) hexyl)-1-((S)-2-(1-fluorocyclopropane-1-carboxamido)-3,3-dimethylbutanoyl)pyrrolidine-2-carboxamide (Intermediate 3)

To a solution of (2S,4R)-4-((tert-butyldimethylsilyl)oxy)-1-((S)-2-(1-fluorocyclopropane-1-carboxamido)-3,3-dimethylbutanoyl)-N—((S)-1-(4-(4-methylthiazol-5-yl)phenyl)-6-oxohexyl)pyrrolidine-2-carboxamide (30.0 mg, 1 Eq, 42.0 μmol) in MeOH (3.00 mL) was added 4-chloro-7,7-dimethyl-9-(piperidin-4-yl)indolo[1,2-a]quinazolin-5 (7H)-one (20.7 mg, 1.3 Eq, 54.5 μmol), sodium cyanotrihydroborate (7.9 mg, 3 Eq, 126 μmol) triethylamine (12.7 mg, 3 Eq, 126 μmol) and the reaction was stirred at 25° C. for 16 hours. LCMS indicated completion of reaction. Water (100 mL) was added, extracted with EA (80 mL×2), the combined organic layers were washed with brine, dried over anhydrous Na₂SO₄ to get tert-butyl (2S,4R)-4-((tert-butyldimethylsilyl)oxy)-N—((S)-6-(4-(4-chloro-7,7-dimethyl-5-oxo-5,7-dihydroindolo[1,2-a]quinazolin-9-yl)piperidin-1-yl)-1-(4-(4-methylthiazol-5-yl)phenyl) hexyl)-1-((S)-2-(1-fluorocyclopropane-1-carboxamido)-3,3-dimethylbutanoyl)pyrrolidine-2-carboxamide (30 mg, 15.8 μmol, 56.96%) as a white solid. LC purity (0.1% TFA): 100% (UV at 254 nm)/MS: 540.0 [M/2+H]; Retention time: 1.69 min.

### Synthesis of (2S,4R)—N—((S)-6-(4-(4-chloro-7,7-dimethyl-5-oxo-5,7-dihydroindolo[1,2-a]quinazolin-9-yl)piperidin-1-yl)-1-(4-(4-methylthiazol-5-yl)phenyl) hexyl)-1-((S)-2-(1-fluorocyclopropane-1-carboxamido)-3,3-dimethylbutanoyl)-4-hydroxypyrrolidine-2-carboxamide (Compound B7)

To a solution of (2S,4R)-4-((tert-butyldimethylsilyl)oxy)-N—((S)-6-(4-(4-chloro-7,7-dimethyl-5-oxo-5,7-dihydroindolo[1,2-a]quinazolin-9-yl)piperidin-1-yl)-1-(4-(4-methylthiazol-5-yl)phenyl) hexyl)-1-((S)-2-(1-fluorocyclopropane-1-carboxamido)-3,3-dimethylbutanoyl)pyrrolidine-2-carboxamide (30.0 mg, 1 Eq, 27.8 μmol) in DCM (3.00 mL) was added 2,2,2-trifluoroacetic acid (3.17 mg, 0.6 mL, 1 Eq, 27.8 μmol), and the reaction was stirred at 25° C. for 2 hours. LCMS indicated completion of reaction. The reaction was concentrated under vacuum. The residue was purified by prep-HPLC, eluted with CH₃CN in H₂O (0.1% TFA) to afford (2S,4R)—N—((S)-6-(4-(4-chloro-7,7-dimethyl-5-oxo-5,7-dihydroindolo[1,2-a]quinazolin-9-yl)piperidin-1-yl)-1-(4-(4-methylthiazol-5-yl)phenyl) hexyl)-1-((S)-2-(1-fluorocyclopropane-1-carboxamido)-3,3-dimethylbutanoyl)-4-hydroxypyrrolidine-2-carboxamide Compound B7 (7.17 mg, 7.00 μmol, 25.2% yield, 94.22% purity) as a white solid. LC purity (0.1% TFA): 95% (UV at 254 nm)/MS: 482.8 [M/2+H]; Retention time: 1.43 min. ¹H NMR (400 MHz, MeOD) δ 8.92 (s, 1H), 8.41 (d, J=8.6 Hz, 1H), 8.10 (d, J=8.6 Hz, 1H), 7.85 (t, J=8.3 Hz, 1H), 7.66 (d, J=7.8 Hz, 1H), 7.56 (s, 1H), 7.47-7.41 (m, 5H), 4.94-4.91 (m, 1H), 4.82-4.81 (m, 1H), 4.65-4.53 (m, 1H), 4.45-4.43 (m, 1H), 3.86-3.85 (m, 1H), 3.78-3.74 (m, 1H), 3.60-3.58 (m, 2H), 3.00-2.93 (m, 5H), 3.35-2.86 (m, 13H), 2.48 (s, 3H), 2.22-2.12 (m, 3H), 2.00-1.70 (m, 7H), 2.09-1.61-1.60 (m, 7H), 1.50-1.45 (m, 3H), 1.32-1.28 (m, 4H), 1.08 (s, 9H).

### Compound B8. (2S,4R)-1-(2-(3-((4-(4-chloro-7,7-dimethyl-5-oxo-5,7-dihydroindolo[1,2-a]quinazolin-9-yl)piperidin-1-yl)methyl)isoxazol-5-yl)-3-methylbutanoyl)-4-hydroxy-N—((S)-1-(4-(4-methylthiazol-5-yl)phenyl)ethyl)pyrrolidine-2-carboxamide

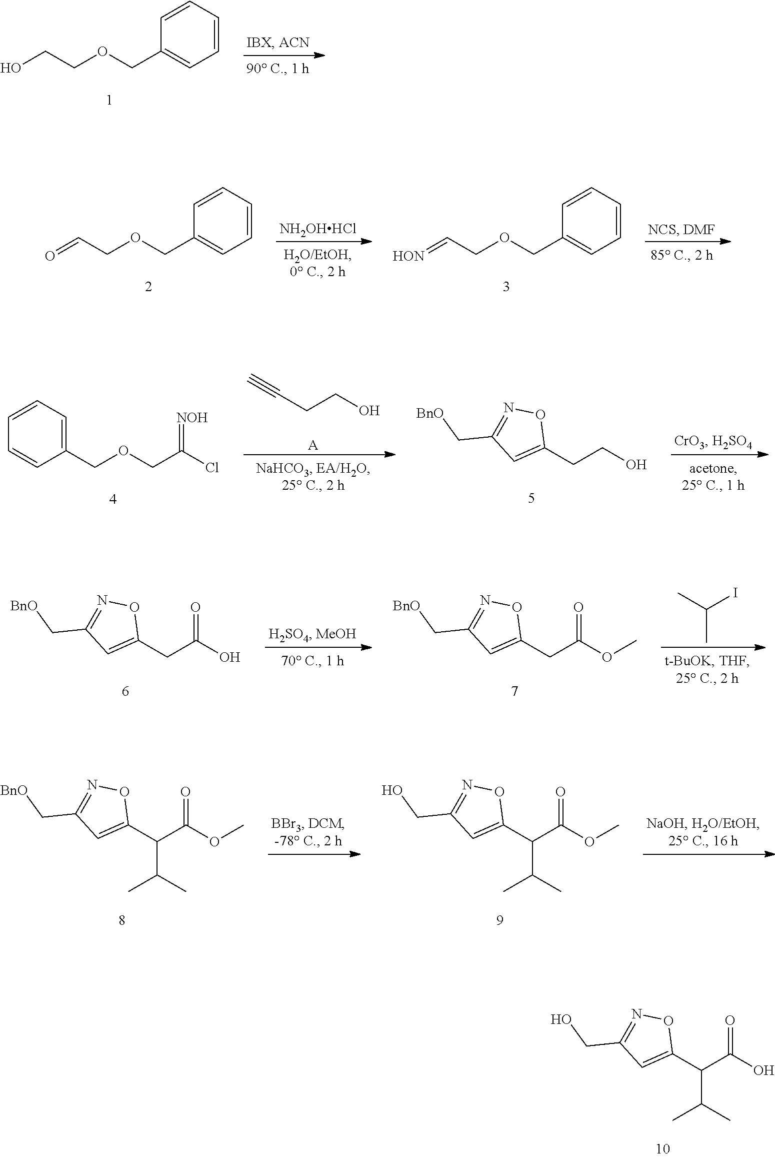

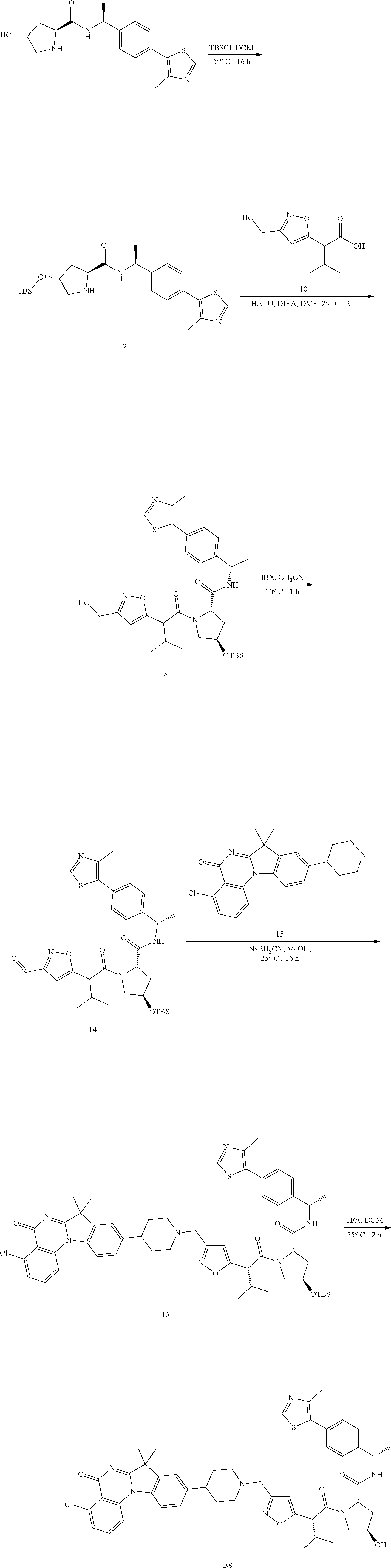

### Synthesis of 2-(benzyloxy) acetaldehyde (Intermediate 2)

To a solution of 2-(benzyloxy) ethan-1-ol (30.0 g, 1 eq, 197 mmol) in acetonitrile (300 mL) was added IBX (110 g, 2 eq, 394 mmol). The mixture was stirred at 90° C. for 1 hour. LCMS indicated completion of reaction. The mixture was filtered, diluted with water (200 mL), extracted with EA (200 mL) and the organic layer was washed with water (200 mL×2), then saturated brine (300 mL). The organic layer was dried over sodium sulfate, filtered, concentrated under vacuum. The resulting residue was purified by silica gel chromatography (100-200 mesh silica gel, 50% of EtOAc in PE) to afford 2-(benzyloxy) acetaldehyde (25.0 g, 166 mmol, 84.4%) as a colorless oil. LC purity (0.1% FA in H₂O, 0.1% FA in ACN): 90.16% (UV at 214 nm)/MS: 151.1 [M+H]; Retention time: 1.33 min.

### Synthesis of (E)-2-(benzyloxy) acetaldehyde oxime (Intermediate 3)

To a solution of 2-(benzyloxy) acetaldehyde (24.0 g, 1 eq, 160 mmol) and hydroxylamine hydrochloride (11.1 g, 1 eq, 160 mmol) in EA (100 mL) and Water (100 mL) was added NaHCO₃ (27 g, 2 eq, 320 mmol). The mixture was stirred at 0° C. for 2 hours. TLC (PE:EA=3:1, R_{f}=0.3) indicated completion of reaction. The mixture was extracted with EA (200 mL), washed with brine (300 mL), dried over sodium sulfate, filtered, concentrated under vacuum to afford (20.0 g, 97 mmol, 61%, 80% purity) as colorless oil. LC purity (0.1% FA in H₂O, 0.1% FA in ACN): 90.16% (UV at 2514 nm)/MS: 200.0 [M+H]; Retention time: 1.21 min.

### Synthesis of (Z)-2-(benzyloxy)-N-hydroxyacetimidoyl chloride (Intermediate 4)

To a solution of (E)-2-(benzyloxy) acetaldehyde oxime (20.0 g, 1 eq, 121 mmol) in DMF (20.0 mL) was added N-Chlorosuccinimide (32.3 g, 19.6 mL, 2 eq, 242 mmol). The mixture was stirred at 90° C. for 2 hours. TLC (PE:EA=3:1, R_{f}=0.4) indicated completion of reaction. The mixture was extracted with EA (200 mL), washed with brine (100 mL), dried over sodium sulfate, filtered, concentrated under vacuum. The resulting residue was purified by silica gel chromatography (100-200 mesh silica gel, 10% of EtOAc in PE) to to get (Z)-2-(benzyloxy)-N-hydroxyacetimidoyl chloride (21.0 g, 84 mmol, 70%, 80% Purity) as a white solid. LC purity (0.1% FA in H₂O, 0.1% FA in ACN): 80.16% (UV at 214 nm)/MS: 200.0 [M+H]; Retention time: 1.21 min.

### Synthesis of compound 2-(3-((benzyloxy)methyl)isoxazol-5-yl) ethan-1-ol (Intermediate 5)

To a solution of (7)-2-(benzyloxy)-N-hydroxyacetimidoyl chloride (22.0 g, 1 eq, 110 mmol) and but-3-yn-1-ol (8.50 g, 1.1 eq, 121 mmol) in ethyl acetate (10.0 mL) and water (10 mL) was added NaHCO₃ (27 g, 3 eq, 331 mmol). The mixture was stirred at 25° C. for 2 hours. LCMS indicated completion of reaction. The mixture was poured into water (800 mL), extracted with ethyl acetate (300 mL×3). The combined organic layers were washed with brine (100 mL), dried over sodium sulfate, filtered and concentrated under vacuum. The residue was purified by silica gel chromatography (100-200 mesh silica gel, 50% of EtOAc in PE) to afford 2-(3-((benzyloxy)methyl)isoxazol-5-yl) ethan-1-ol (7.00 g, 30.0 mmol, 27.2%) as colorless oil. LC purity (0.1% FA): 40.05% (UV at 214 nm)/MS: 234.11 [M+1]; Retention time: 1.50 min.

### Synthesis of compound 2-[3-(3-phenylmethoxypropyl)-1,2-oxazol-5-yl]acetic acid (Intermediate 6)

To a solution of 2-(3-((benzyloxy)methyl)isoxazol-5-yl) ethan-1-ol (4.50 g, 1 eq, 19.3 mmol) in acetone (10 mL), was added CrO₃ (5.79 g, 3 eq, 57.9 mmol) and H₂SO₄ (397 mg, 0.216 mL, 0.223 eq, 4.05 mmol) under N₂. The mixture was stirred at 25° C. for 2 hours. TLC (PE:EtOAc=1:1, Rf=0.5) indicated completion of reaction. The mixture was poured into water (200 mL), extracted with ethyl acetate (100 mL×3). The combined organic layers were washed with brine (50 mL), dried over sodium sulfate, filtered and concentrated under vacuum. The residue was purified by silica gel chromatography (100-200 mesh silica gel, 50% of EtOAc in PE) to afford 2-[3-(3-phenylmethoxypropyl)-1,2-oxazol-5-yl]acetic acid (1000 mg, 3.55 mmol, 71.5% yield) as colorless oil. ¹HNMR (CD₃OD-d₄, 400 MHz): δ 12.858 (s, 1H), 7.284-7.387 (m, 5H), 6.334-6.445 (s, 1H), 4.483-4.647 (m, 4H), 3.884-4.031 (m, 2H).

### Synthesis of compound methyl 2-(3-((benzyloxy)methyl)isoxazol-5-yl)acetate (Intermediate 7)

To a solution of 2-(3-((benzyloxy)methyl)isoxazol-5-yl) acetic acid (4.50 g, 1 eq, 18.2 mmol) in MeOH (5 mL) and was added con. H₂SO₄ (397 mg, 0.216 mL, 0.223 eq, 4.1 mmol), the reaction was stirred at 70° C. for 2 hours. LCMS indicated completion of reaction. The mixture was poured into water (200 mL), extracted with ethyl acetate (100 mL×3). The combined organic layers were washed with brine (50 mL), dried over sodium sulfate, filtered and concentrated under vacuum. The residue was purified by silica gel chromatography (100-200 mesh silica gel, 50% of EtOAc in PE) to afford methyl 2-(3-((benzyloxy)methyl)isoxazol-5-yl)acetate (2.30 g, 8.80 mmol, 48.4%) as colorless oil. LC purity (0.1% FA): 55.72% (UV at 214 nm)/MS: 262.10 [M+1]; Retention time: 1.67 min.

### Synthesis of compound methyl 2-(3-((benzyloxy)methyl)isoxazol-5-yl)-3-methylbutanoate (Intermediate 8)

To a solution of methyl 2-(3-((benzyloxy)methyl)isoxazol-5-yl)acetate (2.30 g, 1 eq, 8.80 mmol) in THF (200 mL) was added t-BuOK (1.97 g, 2 eq, 17.6 mmol) and 2-iodopropane (2.99 g, 2 eq, 17.6 mmol) dropwise at 0° C. under N₂. The solution was stirred at rt for 16 hours. LCMS indicated completion of reaction. The mixture was poured into water (200 mL), extracted with ethyl acetate (100 mL×3). The combined organic layers were washed with brine (50 mL), dried over sodium sulfate, filtered and concentrated under vacuum to afford the crude methyl 2-(3-((benzyloxy)methyl)isoxazol-5-yl)-3-methylbutanoate (1.40 g, 4.61 mmol, 52.4%) as colorless oil. LC purity (0.1% TFA): 58.79% (UV at 214 nm)/MS: 304.15 [M+1]; Retention time: 1.77 min.

### Synthesis of 2-(3-(hydroxymethyl)isoxazol-5-yl)-3-methylbutanoate (Intermediate 9)

To a solution of methyl 2-(3-((benzyloxy)methyl)isoxazol-5-yl)-3-methylbutanoate (1.40 g, 1 eq, 4.6 mmol) in DCM (20.0 mL) was added BBr₃ (2.31 g, 873 μL, 2 eq, 9.2 mmol) dropwise at −78° C. under N₂. The reaction was stirred at −78° C. for 2 hours. LCMS indicated completion of reaction. The reaction was quenched by water (50 mL), extracted with DCM (30 mL×2). The organic layer was dried over sodium sulfate, filtered and concentrated under vacuum. The resulting residue was purified by silica gel chromatography (100-200 mesh silica gel, 30% of EtOAc in PE) to afford methyl 2-(3-(hydroxymethyl)isoxazol-5-yl)-3-methylbutanoate (640 mg, 3.00 mmol, 65.0%) as colorless oil. LC purity (0.1% TFA): 71.40% (UV at 214 nm)/MS: 214.10 [M+1]; Retention time: 1.79 min.

### Synthesis of compound 2-(3-(hydroxymethyl)isoxazol-5-yl)-3-methylbutanoic acid (Intermediate 10)

To a solution of methyl 2-(3-(hydroxymethyl)isoxazol-5-yl)-3-methylbutanoate (640 mg, 1 eq, 3.00 mmol) in MeOH (20.0 mL) was added potassium hydroxide (505 mg, 3 eq, 9.00 mmol) in water (3 mL), the reaction was stirred at 25° C. for 16 hours. LCMS indicated completion of reaction. The reaction was adjusted pH to 7 with 2M HCl and extracted with EA (150 mL). The organic layer was dried over sodium sulfate, filtered and concentrated to afford 2-(3-(hydroxymethyl)isoxazol-5-yl)-3-methylbutanoic acid (420 mg, 2.11 mmol, 70.4%) as colorless oil. LC purity (0.1% FA in H₂O, 0.1% FA in ACN): 75.20% (UV at 214 nm)/MS: 200.21 [M+1]; Retention time: 1.41 min.

### Synthesis of (2S,4R)-4-((tert-butyldimethylsilyl)oxy)-N—((S)-1-(4-(4-methylthiazol-5-yl)phenyl)ethyl)pyrrolidine-2-carboxamide (Intermediate 12)

To a solution of (2S,4R)-4-hydroxy-N—((S)-1-(4-(4-methylthiazol-5-yl)phenyl)ethyl)pyrrolidine-2-carboxamide (2.00 g, 1 eq, 6.04 mmol) and imidazole (1.35 g, 4 eq, 18.0 mmol) in DCM (15 mL) was added TBSCl (2.23 g, 3 eq, 18.1 mmol). The reaction was stirred at 25° C. for 16 hours. LCMS indicated completion of reaction. The reaction was poured into water (100 mL), extracted with DCM (8 mL×3). The combined organic layers were dried over sodium sulfate, filtered and concentrated under vacuum. The resulting residue was purified by silica gel chromatography (100-200 mesh silica gel, 50% of EtOAc in PE) to give (2S,4R)-4-((tert-butyldimethylsilyl)oxy)-N—((S)-1-(4-(4-methylthiazol-5-yl)phenyl)ethyl)pyrrolidine-2-carboxamide (1.69 g, 3.79 mmol, 6 2.8%) as a white solid. LC purity (0.1% TFA): 100% (UV at 254 nm)/MS: 446.2 [M+1]; Retention time: 1.67 min.

### Synthesis of compound (2S,4R)-4-((tert-butyldimethylsilyl)oxy)-1-(2-(3-(hydroxymethyl) isoxazol-5-yl)-3-methylbutanoyl)-N—((S)-1-(4-(4-methylthiazol-5-yl)phenyl)ethyl)pyrrolidine-2-carboxamide (Intermediate 13)

To a solution of 2-(3-(hydroxymethyl)isoxazol-5-yl)-3-methylbutanoic acid (500 mg, 1 eq, 2.51 mmol) in DMF (5.00 mL) was added (2S,4R)-4-((tert-butyldimethylsilyl)oxy)-N—((S)-1-(4-(4-methylthiazol-5-yl)phenyl)ethyl)pyrrolidine-2-carboxamide (1.12 g, 1 eq, 2.51 mmol), HATU (1.91 g, 2 eq, 5.02 mmol). then the mixture was stirred at 25° C. for 16 hours. LCMS indicated completion of reaction. The mixture was poured into water (100 mL), extracted with ethyl acetate (50 mL×3). The combined organic layers were washed with brine (30 mL), dried over sodium sulfate, filtered and concentrated under vacuum to afford methylbutanoyl)-N—((S)-1-(4-(4-methylthiazol-5-yl)phenyl)ethyl)pyrrolidine-2-carboxamide (1500 mg, 2.393 mmol, 95.3%) as yellow oil. LC purity (0.1% FA): 96.1% (UV at 214 nm)/MS: 627.30 [M+1]; Retention time: 1.18 min

### Synthesis of (2S,4R)-4-((tert-butyldimethylsilyl)oxy)-1-(3-methyl-2-(3-(2-oxoethyl)isoxazol-5-yl)butanoyl)-N—((S)-1-(4-(4-methylthiazol-5-yl)phenyl)ethyl)pyrrolidine-2-carboxamide (Intermediate 14)

To a solution of (2S,4R)-4-((tert-butyldimethylsilyl)oxy)-1-(2-(3-(hydroxymethyl)isoxazol-5-yl)-3-methylbutanoyl)-N—((S)-1-(4-(4-methylthiazol-5-yl)phenyl)ethyl)pyrrolidine-2-carboxamide (50.0 mg, 1 eq, 0.08 mmol) in acetonitrile (20.0 mL) was added IBX (33.5 mg, 1.5 eq, 0.12 mmol). The mixture was stirred at 80° C. for 1 hour. LCMS indicated completion of reaction. The mixture was filtered and the filtrate was concentrated under vacuum to afford (2S,4R)-4-((tert-butyldimethylsilyl)oxy)-1-(2-(3-formylisoxazol-5-yl)-3-methylbutanoyl)-N—((S)-1-(4-(4-methylthiazol-5-yl)phenyl)ethyl)pyrrolidine-2-carboxamide (50.0 mg, 0.08 mmol, 98.1% yield) as yellow oil. LC purity (0.1% FA): 68.16% (UV at 254 nm)/MS: 624.87 [M+H]; Retention time: 1.82 min.

### Synthesis of (2S,4R)-4-((tert-butyldimethylsilyl)oxy)-1-((R)-2-(3-((4-(4-chloro-7,7-dimethyl-5-oxo-5,7-dihydroindolo[1,2-a]quinazolin-9-yl)piperidin-1-yl)methyl)isoxazol-5-yl)-3-methylbutanoyl)-N—((S)-1-(4-(4-methylthiazol-5-yl)phenyl)ethyl)pyrrolidine-2-carboxamide (Intermediate 16)

To a solution of (2S,4R)-4-((tert-butyldimethylsilyl)oxy)-1-(2-(3-formylisoxazol-5-yl)-3-methylbutanoyl)-N—((S)-1-(4-(4-methylthiazol-5-yl)phenyl)ethyl)pyrrolidine-2-carboxamide (50.0 mg, 1 eq, 0.08 mmol) in MeOH (2.00 mL) was added 4-chloro-7,7-dimethyl-9-(piperidin-4-yl)indolo[1,2-a]quinazolin-5 (7H)-one (30.4 mg, 1 eq, 0.08 mmol). After 30 mins, NaCNBH₃ (15.1 mg, 3 eq, 0.24 mmol) was added. The mixture was stirred at 25° C. for 16 hours. LCMS indicated completion of reaction. The mixture was filtered and the filtrate was concentrated under vacuum to afford (2S,4R)-4-((tert-butyldimethylsilyl)oxy)-1-((R)-2-(3-((4-(4-chloro-7,7-dimethyl-5-oxo-5,7-dihydroindolo[1,2-a]quinazolin-9-yl)piperidin-1-yl)methyl)isoxazol-5-yl)-3-methylbutanoyl)-N—((S)-1-(4-(4-methylthiazol-5-yl)phenyl)ethyl)pyrrolidine-2-carboxamide (30.0 mg, 0.030 mmol, 37.9% yield) as colorless oil. LC purity (0.1% FA): 32.83% (UV at 254 nm)/MS: 988.76 [M+H]; Retention time: 1.54 min.

### Synthesis of (2S,4R)-1-(2-(3-((4-(4-chloro-7,7-dimethyl-5-oxo-5,7-dihydroindolo[1,2-a]quinazolin-9-yl)piperidin-1-yl)methyl)isoxazol-5-yl)-3-methylbutanoyl)-4-hydroxy-N—((S)-1-(4-(4-methylthiazol-5-yl)phenyl)ethyl)pyrrolidine-2-carboxamide

To a solution of (2S,4R)-4-((tert-butyldimethylsilyl)oxy)-1-(2-(3-((4-(4-chloro-7,7-dimethyl-5-oxo-5,7-dihydroindolo[1,2-a]quinazolin-9-yl)piperidin-1-yl)methyl)isoxazol-5-yl)-3-methylbutanoyl)-N—((S)-1-(4-(4-methylthiazol-5-yl)phenyl)ethyl)pyrrolidine-2-carboxamide (30.0 mg, 1 eq, 0.03 mmol) in DCM (1.00 mL) was added TFA (1.00 mL). The mixture was stirred at 25° C. for 2 hours. LCMS indicated completion of reaction. The mixture was filtered and concentrated under vacuum. The residue was purified by prep-HPLC, eluted with CH₃CN in H₂O (0.1% TFA) to afford (2S,4R)-1-(2-(3-((4-(4-chloro-7,7-dimethyl-5-oxo-5,7-dihydroindolo[1,2-a]quinazolin-9-yl)piperidin-1-yl)methyl)isoxazol-5-yl)-3-methylbutanoyl)-4-hydroxy-N—((S)-1-(4-(4-methylthiazol-5-yl)phenyl)ethyl)pyrrolidine-2-carboxamide Compound B8 (2.21 mg, 0.002 mmol, 7.65% yield, 91.9% purity) as a white solid. LC purity (0.03% NH₃·H₂O): 91.90% (UV at 214 nm)/MS: 874.5 [M+H]; Retention time: 7.874 min. ¹H NMR (400 MHz, MeOD) δ 8.65-8.60 (m, 1H), 8.40-8.38 (m, 1H), 8.10-8.09 (m, 1H), 7.86-7.85 (m, 1H), 7.66-7.64 (m, 1H), 7.57-7.55 (m, 4H), 7.46-7.32 (m, 3H), 6.59 (s, 1H), 4.59-4.37 (m, 5H), 4.10-4.08 (m, 1H), 3.95 (d, J=9.6 Hz, 1H), 3.85 (m, 1H), 3.76 (m, 2H), 3.70-3.59 (m, 2H), 3.52-3.44 (m, 1H), 3.17-3.07 (m, 1H), 2.50-2.35 (m, 5H), 2.25-2.23 (m, 3H), 2.10-2.08 (m, 2H), 2.03-1.88 (m, 2H), 1.58 (m, 2H), 1.53-1.52 (m, 3H), 1.29 (m, 1H), 1.10 (m, 3H), 0.94-0.89 (m, 3H).

### Compound B9. (2S,4R)-1-((R)-2-(3-(4-((4-(4-chloro-7,7-dimethyl-5-oxo-5,7-dihydroindolo[1,2-a]quinazolin-9-yl)piperidin-1-yl)methyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide

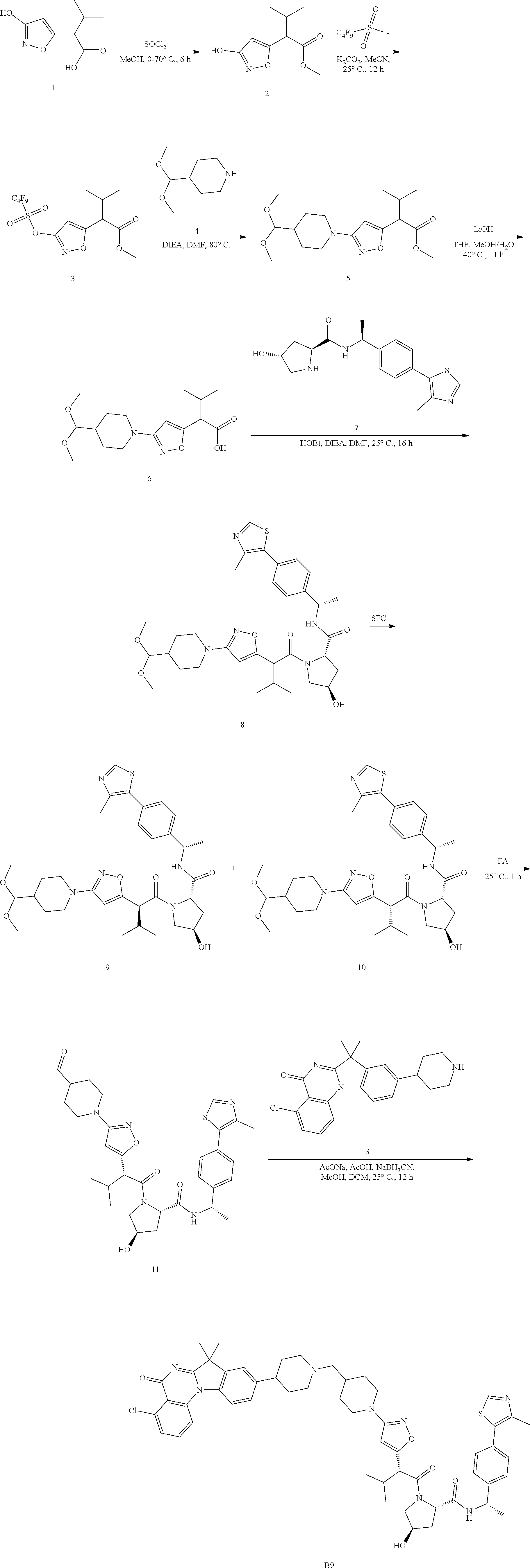

### Synthesis of crude methyl 2-(3-hydroxyisoxazol-5-yl)-3-methylbutanoate (Intermediate 2)

To a solution of 2-(3-hydroxyisoxazol-5-yl)-3-methylbutanoic acid (3.00 g, 1 Eq, 16.2 mmol) in MeOH (250 mL) was added SOCl₂ (1.92 g, 1.0 Eq, 16.2 mmol) dropwise. The reaction was stirred at 70° C. for 6 hours. LCMS indicated completion of reaction. Water (100 mL) was added, extracted with EA (200 mL), the organic layer was washed with brine (100 mL), dried over anhydrous Na₂SO₄, filtered and concentrated under vacuum to get methyl 2-(3-hydroxyisoxazol-5-yl)-3-methylbutanoate (2.30 g, 11.5 mmol, 71.3%) as a green oil. LC purity (0.1% FA): 95.00% (UV at 254 nm)/MS: 200.0 [M+H]; Retention time: 1.240 min.

### Synthesis of methyl 3-methyl-2-(3-(((perfluorobutyl) sulfonyl)oxy)isoxazol-5-yl) butanoate (Intermediate 3)

To a solution of methyl 2-(3-hydroxyisoxazol-5-yl)-3-methylbutanoate (3.20 g, 1.0 eq, 16.1 mmol) and Potassium carbonate (1.11 g, 0.5 eq, 8.03 mmol) in MeCN (20.0 mL) was added 1,1,2,2,3,3,4,4,4-nonafluorobutane-1-sulfonyl fluoride (9.71 g, 2.0 Fq, 32.1 mmol) dropwise at 0° C. under N₂. The reaction was stirred at 15° C. for 16 hours. LCMS showed 40% of methyl 3-methyl-2-(3-(((perfluorobutyl) sulfonyl)oxy)isoxazol-5-yl)butanoate was detected. The reaction mixture was diluted with water (100 mL), extracted with EA (50 mL). The resulting organic layer was washed with brine (50 mL), dried over anhydrous Na₂SO₄, filtered and concentrated under vacuum. The resulting residue was purified by flash, eluted with MTBE in PE (0%˜40%) to get methyl 3-methyl-2-(3-(((perfluorobutyl) sulfonyl)oxy)isoxazol-5-yl)butanoate (3.70 g, 7.69 mmol, 47.9%). LC purity (0.1% FA): 94.00% (UV at 254 nm)/MS: 482.0 [M+H]; Retention time: 1.312 min.

### Synthesis of methyl methyl 2-(3-(4-(dimethoxymethyl)piperidin-1-yl)isoxazol-5-yl)-3-methylbutanoate (Intermediate 5)

To a solution of methyl 3-methyl-2-(3-(((4,4,4,4,4,4,4,4,4-nonafluoro-4112-buta-1,3-diyn-1-yl) sulfonyl)oxy)isoxazol-5-yl)butanoate (3.70 g, 1.0 eq, 7.69 mmol) in DMF (74.0 mL) was added 4-(dimethoxymethyl)piperidine (3.06 g, 2.5 eq, 19.2 mmol) and DIEA (2.98 g, 3.0 Eq, 23.1 mmol). The mixture was stirred at 80° C. for 4 hours. LCMS indicated completion of reaction. The mixture was poured into H₂O and extracted with EA (50 mL), then washed with brine (50 mL), dried over sodium sulfate, filtered and concentrated under vacuum. The resulting residue was purified by silica gel column chromatography (100-200 mesh silica gel, 0%˜30% of MTBE in PE) to obtain methyl methyl 2-(3-(4-(dimethoxymethyl)piperidin-1-yl)isoxazol-5-yl)-3-methylbutanoate (1.19 g, 3.50 mmol, 45.5%) as a yellow oil. LC purity (0.1% FA): 96.00% (UV at 254 nm)/MS: 341.2 [M+H]; Retention time: 1.586 min. ¹H NMR (400 MHz, CDCl₃) δ 5.92 (d, J=1.9 Hz, 1H), 4.06 (d, J=4.8 Hz, 1H), 3.72 (dd, J=6.5, 4.5 Hz, 5H), 3.47 (dd, J=8.8, 1.8 Hz, 1H), 3.42-3.30 (m, 6H), 2.81 (t, J=12.4 Hz, 2H), 2.33 (td, J=13.1, 6.4 Hz, 1H), 1.80 (d, J=12.2 Hz, 3H), 1.48-1.25 (m, 2H), 0.99 (dd, J=6.5, 1.8 Hz, 3H), 0.92 (dd, J=6.6, 1.8 Hz, 3H).

### Synthesis of 2-(3-(4-(dimethoxymethyl)piperidin-1-yl)isoxazol-5-yl)-3-methylbutanoic acid (Intermediate 6)

To a solution of methyl 2-(3-(4-(dimethoxymethyl)piperidin-1-yl)isoxazol-5-yl)-3-methylbutanoate (790 mg, 1 Eq, 2.32 mmol) in THF (2.30 mL) and MeOH (2.30 mL) was added lithium hydroxide (108 mg, 1.95 Eq, 4.53 mmol) in Water (2.30 mL). The mixture was stirred at 40° C. for 11 hours. LCMS indicated completion of reaction. The reaction was concentrated, then poured into H₂O and added TFA to pH=5-6. The resulting residue was purified by prep-HPLC to get 2-(3-(4-(dimethoxymethyl)piperidin-1-yl)isoxazol-5-yl)-3-methylbutanoic acid (0.35 g, 1.1 mmol, 45%) as a colourless oil. LC purity (0.03% TFA): 98.00% (UV at 254 nm)/MS: 327.2 [M+H]; Retention time: 0.984 min.

### Synthesis of (2S,4R)-1-(2-(3-(4-(dimethoxymethyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (Intermediate 10)

To a solution of 2-(3-(4-(dimethoxymethyl)piperidin-1-yl)isoxazol-5-yl)-3-methylbutanoic acid (800 mg, 1 Eq, 2.45 mmol) in DMF (18.0 mL) was added (2S,4R)-4-((tert-butyldimethylsilyl)oxy)-N—((S)-1-(4-(4-methylthiazol-5-yl)phenyl)ethyl)pyrrolidine-2-carboxamide (1.31 g, 1.2 Eq, 2.94 mmol), EDCl (612 mg, 1.3 Eq, 3.19 mmol), HOBT (430 mg, 1.3 Eq, 3.19 mmol) and DIEA (949 mg, 3 Eq, 7.35 mmol). The mixture was stirred at 25° C. for 16 hours. LCMS indicated completion of reaction. The reaction was poured into H₂O and extracted with EA (100 mL), then washed brine and dried over sodium sulfate, filtered and concentrated under vacuum. The resulting residue was purified by silica gel column chromatography (100-200 mesh silica gel, 70% of EA in PE) to get (2S,4R)-1-(2-(3-(4-(dimethoxymethyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (900 mg, 1.4 mmol, 56%) as a yellow oil. LC purity (0.03% TFA): 98% (UV at 254 nm)/MS: 639.81 [M+H]; Retention time: 3.506 min. Then the (2S,4R)-1-[2-[3-[4-(dimethoxymethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (900 mg, 1.0 eq, 1.41 mmol) was serrated by chiral SFC (column: DAICEL CHIRALPAK AD (250 mm*30 mm, 10 μm); mobile phase: [0.1% NH₄OH/IPA]; B %: 45%) to afford (2S,4R)-1-[(2S)-2-[3-[4-(dimethoxymethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (378 mg, 0.59 mmol, 42% yield) (analytical retention time 1.88 min) as a yellow oil and (2S,4R)-1-[(2R)-2-[3-[4-(dimethoxymethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (400 mg, 0.63 mmol, 44% yield) as a yellow oil.

### Synthesis of (2S,4R)-1-((R)-2-(3-(4-formylpiperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (Intermediate 11)

A solution of (2S,4R)-1-((R)-2-(3-(4-(dimethoxymethyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (50.0 mg, 1.0 Eq, 78.1 μmol) in FA (2.00 mL) was stirred at 25° C. for 2 h. LCMS indicated completion of reaction. The reaction was concentrated under vacuum to afford (2S,4R)-1-((R)-2-(3-(4-formylpiperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (50.0 mg, 67 μmol, 86%) as a yellow oil. LC purity (0.1% FA): 90.00% (UV at 254 nm)/MS: 594.7 [M+H]. Retention time: 1.270 min.

### Synthesis of (2S,4R)-1-((R)-2-(3-(4-((4-(4-chloro-7,7-dimethyl-5-oxo-5,7-dihydroindolo[1,2-a]quinazolin-9-yl)piperidin-1-yl)methyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound B9)

To a solution of (2S,4R)-1-((R)-2-(3-(4-formylpiperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (50.0 mg, 1.0 Eq, 84.2 μmol) in MeOH (2.00 mL) was added TEA (25.6 mg, 35.2 μL, 3.0 Eq, 253 μmol), 4-chloro-7,7-dimethyl-9-(piperidin-4-yl)indolo[1,2-a]quinazolin-5 (7H)-one (32.0 mg, 1.0 Eq, 84.2 μmol) and stirred at 25° C. for 0.5 hour. Then NaCNBH₄ (26.5 mg, 5.0 Eq, 421 μmol) was added to above mixture. The mixture was stirred at 25° C. for 16 hours. L LCMS indicated completion of reaction. The reaction was poured into water (10 mL) and extracted with EA (20 mL), then washed with brine (10 mL) and dried over sodium sulfate, filtered and concentrated under vacuum. The residue was purified by prep-HPLC, eluted with MeCN in H₂O from 10% to 95% (0.1% NH₄HCO₃) to afford (2S,4R)-1-((R)-2-(3-(4-((4-(4-chloro-7,7-dimethyl-5-oxo-5,7-dihydroindolo[1,2-a]quinazolin-9-yl)piperidin-1-yl)methyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (2.25 mg, 2.23 μmol, 2.65%, 95% Purity) as a purple solid. LC purity (0.01% FA): 100.00% (UV at 254 nm)/MS: 958.63 [M+H]; Retention time: 1.33 min. ¹H NMR (400 MHz, MeOD) δ 8.87 (s, 1H), 8.43 (d, J=8.1 Hz, 1H), 8.07 (d, J=8.5 Hz, 1H), 7.84 (t, J=8.3 Hz, 1H), 7.65 (d, J=7.3 Hz, 1H), 7.56 (s, 1H), 7.50-7.29 (m, 5H), 6.08 (d, J=21.4 Hz, 1H), 5.04 (d, J=6.6 Hz, 1H), 4.50 (d, J=8.3 Hz, 1H), 4.44 (s, 1H), 3.85 (d, J=6.7 Hz, 1H), 3.70 (d, J=12.1 Hz, 2H), 3.66-3.55 (m, 2H), 3.48 (s, 1H), 3.36 (s, 2H), 3.11 (d, J=18.7 Hz, 2H), 2.87 (t, J=11.7 Hz, 2H), 2.73-2.62 (m, 1H), 2.48 (s, 3H), 2.37 (s, 1H), 2.31 (s, 2H), 2.15 (s, 3H), 1.88 (s, 4H), 1.85 (s, 2H), 1.61 (s, 6H), 1.52 (d, J=7.1 Hz, 2H), 1.31 (s, 2H), 1.05 (d, J=6.7 Hz, 3H), 0.90 (t, J=9.4 Hz, 3H).

### Compound B10. (2S,4R)-1-((R)-2-(3-(4-((4-(4-chloro-7,7-dimethyl-5-oxo-5,7-dihydroindolo[1,2-a]quinazolin-8-yl)piperidin-1-yl)methyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide

Compound B10 was synthesized employing a procedure analogous to that described for B9. LC purity (0.1% FA): 94.84% (UV at 254 nm)/MS: 957.4 [M+H]; Retention time: 1.38 min. ¹H NMR (400 MHz, DMSO-d6) δ 8.97-8.98 (m, 1H), 8.38-8.43 (m, 2H), 8.03-8.04 (s, 1H), 7.63-7.65 (m, 1H), 7.52-7.55 (m, 1H), 7.45-7.69 (m, 1H), 7.39-7.43 (m, 2H), 7.36-7.37 (m, 2H), 7.15-7.18 (m, 1H), 6.17-6.19 (m, 1H), 5.11-5.13 (m, 1H), 4.90-4.94 (m, 1H), 4.35-4.39 (m, 1H), 4.28-4.29 (m, 1H), 3.63-3.68 (m, 4H), 3.57-3.59 (m, 1H), 3.40-3.42 (m, 1H), 3.28-3.32 (m, 1H), 2.79-2.85 (m, 1H), 2.45 (s, 3H), 2.21-2.35 (m, 2H), 1.97-2.04 (m, 2H), 1.67-1.85 (m, 5H), 1.57 (s, 6H), 1.42-1.56 (m, 2H), 1.39-1.41 (m, 3H), 1.23-1.27 (m, 2H), 0.95-0.99 (m, 3H), 0.78-0.83 (m, 3H)

The examples in the table below were prepared according to the same method as Compound B2 using appropriate starting materials.

| | Calcd. | Found | |
|---|---|---|---|
| Compound | (M + H)⁺ | (M + H)⁺ | |
| No. | m/z | m/z | ¹H NMR |
| B18 | 958.4 | 958.4 | ¹H NMR (400 MHz, CD3OD) δ 8.88 (s, 1H), 8.41 |
| | | | (d, J = 8.8 Hz, 1H), 8.13 (d, J = 8.4 Hz, 1H), 7.85 |
| | | | (t, J = 8.0 Hz, 1H), 7.67 (d, J = 7.6 Hz, 1H), 7.56 |
| | | | (s, 1H), 7.49-7.34 (m, 5H), 5.01 (d, J = 7.6 Hz, |
| | | | 1H), 4.73-4.71 (m, 1H), 4.57-4.56 (m, 1H), 4.45- |
| | | | 4.43 (m, 1H), 3.92-3.68 (m, 5H), 3.48 (s, 3H), 2.47 |
| | | | (d, J = 5.2 Hz, 3H), 2.17 (s, 3H), 2.05-2.02 (m, |
| | | | 4H), 1.83-1.76 (m, 10H), 1.62 (s, 6H), 1.51 (d, J = |
| | | | 7.2 Hz, 3H), 1.03 (s, 9H). |
| B23 | 930.4 | 930.6 | ¹H NMR (400 MHz, CD3OD) δ 8.88 (s, 1H), 8.42- |
| | | | 8.40 (m, 1H), 8.10-8.08 (m, 1H), 7.85-7.84 (m, |
| | | | 1H), 7.66-7.64 (m, 1H), 7.58 (s, 1H), 7.48-7.35 (m, |
| | | | 5H), 5.03-5.01 (m, 2H), 4.69-4.66 (m, 2H), 4.56- |
| | | | 4.54 (m, 6H), 4.44 (s, 1H), 3.85-3.72 (m, 2H), |
| | | | 3.53-3.51 (m, 2H), 3.18-3.16 (m, 2H), 2.91 (s, 3H), |
| | | | 2.48 (s, 3H), 2.07-2.04 (m, 4H), 1.96-1.94 (m, 1H), |
| | | | 1.64-1.57 (m, 6H), 1.52-1.50 (m, 3H), 1.02 (s, 9H). |
| B40 | 946.4 | 946.5 | ¹H NMR (400 MHz, CD3OD) δ 8.89 (s, 1H), 8.48- |
| | | | 8.42 (m, 1H), 7.99 (s, 1H), 7.96-7.86 (m, 1H), 7.78 |
| | | | (d, J = 8.0 Hz, 1H), 7.68-7.61 (m, 1H), 7.42-7.36 |
| | | | (m, 4H), 7.34-7.32 (m, 1H), 5.08-4.98 (m, 1H), |
| | | | 4.63-4.61 (m, 1H), 4.54-4.47 (m, 2H), 4.44-4.42 |
| | | | (m, 1H), 3.88-3.86 (m, 2H), 3.72-3.70 (m, 1H), |
| | | | 3.14-3.08 (m, 2H), 2.48 (s, 3H), 2.38-2.24 (m, 2H), |
| | | | 2.18-2.10 (m, 2H), 2.06-1.94 (m, 5H), 1.59 (s, 6H), |
| | | | 1.58-1.51 (m, 3H), 1.29 (s, 1H), 1.24-1.16 (m, 6H), |
| | | | 1.05 (s, 9H), 1.04-0.90 (m, 5H). |
| B65 | 944.5 | 944.6 | ¹H NMR (400 MHz, DMSO) δ 8.44 (dd, J = 17.4, |
| | | | 8.2 Hz, 2H), 7.85 (t, J = 8.3 Hz, 1H), 7.71 (t, J = |
| | | | 8.3 Hz, 2H), 7.63 (dd, J = 7.8, 3.9 Hz, 2H), 7.46 (d, |
| | | | J = 8.2 Hz, 2H), 7.32-7.23 (m, 2H), 7.13 (s, 1H), |
| | | | 7.00 (s, 1H), 5.10 (s, 1H), 4.96 (t, J = 7.2 Hz, 1H), |
| | | | 4.62 (d, J = 9.0 Hz, 1H), 4.56-4.39 (m, 2H), 4.28 (s, |
| | | | 1H), 4.12 (s, 1H), 3.96 (s, 3H), 3.58 (t, J = 10.2 Hz, |
| | | | 2H), 3.13 (s, 1H), 2.99 (s, 1H), 2.62 (d, J = 12.2 |
| | | | Hz, 2H), 2.36 (d, J = 21.1 Hz, 1H), 2.01 (d, J = 8.6 |
| | | | Hz, 1H), 1.89 (s, 2H), 1.79 (s, 2H), 1.71 (s, 4H), |
| | | | 1.57 (s, 1H), 1.50 (s, 6H), 1.47 (s, 2H), 1.34 (t, J = |
| | | | 32.9 Hz, 5H), 0.94 (s, 9H). |
| B70 | 990.4 | 990.4 | ¹H NMR (400 MHz, CD3OD) δ 8.87 (s, 1H), 8.48- |
| | | | 8.46 (m, 1H), 7.97 (s, 1H), 7.91-7.90 (m, 1H), 7.77- |
| | | | 7.75 (m, 1H), 7.59-7.56 (m, 1H), 7.46-7.39 (m, |
| | | | 4H), 7.35-7.33 (m, 1H), 5.02-5.00 (m, 1H), 4.63- |
| | | | 4.53 (m, 2H), 4.44 (s, 1H), 3.87-3.85 (m, 1H), 3.76- |
| | | | 3.74 (m, 1H), 2.94-2.84 (m, 1H), 2.38-2.31 (m, |
| | | | 9H), 2.23-2.15 (m, 1H), 2.07-1.85 (m, 10H), 1.59 |
| | | | (s, 6H), 1.52-1.50 (m, 4H), 1.12-1.10 (m, 2H), 1.04 |
| | | | (s, 9H), 1.02-1.00 (m, 2H). |
| B76 | 1000.4 | 1000.6 | ¹H NMR (400 MHz, CD₃OD) δ 8.91-8.90 (m, 1H), |
| | | | 8.40 (d, J = 8.8 Hz, 1H), 8.17 (d, J = 8.8 Hz, 1H), |
| | | | 7.90-7.86 (m, 1H), 7.73-7.69 (m, 2H), 7.62-7.59 |
| | | | (m, 1H), 7.46-7.38 (m, 4H), 5.02-4.9(m, 1H), 4.64- |
| | | | 4.55 (m, 2H), 4.44 (s, 1H), 3.87-3.85 (m, 1H), 3.78- |
| | | | 3.74 (m, 3 H), 3.57-3.48 (m, 1H), 3.19-3.05 (m, |
| | | | 5H), 2.48-2.47 (m, 3H), 2.40 (m, 1H, 2.23-2.20 |
| | | | (m, 3H), 2.12-2.07 (m, 2H), 1.99-1.93(m, 8H), |
| | | | 1.60-1.57 (m, 2H), 1.54-1.50 (m, 3H), 1.19-1.12 |
| | | | (m, 2H), 1.05 (s, 9H). |
| B79 | 958.4 | 958.4 | ¹H NMR (400 MHz, CD3OD) δ 8.88 (s, 1H), 8.42 |
| | | | (d, J = 8.7 Hz, 1H), 8.09 (d, J = 8.5 Hz, 1H), 7.85 |
| | | | (t, J = 8.3 Hz, 1H), 7.66 (d, J = 7.9 Hz, 1H), 7.57- |
| | | | 7.51 (m, 2H), 7.47-7.41 (m, 4H), 6.12-5.98 (m, |
| | | | 1H), 5.53 (d, J = 5.6 Hz, 1H), 5.39 (d, J = 17.1 Hz, |
| | | | 1H), 5.26 (dd, J = 10.4, 9.1 Hz, 2H), 5.17 (s, 2H), |
| | | | 4.63-4.61 (m, 1H), 4.49 (d, J = 20.4 Hz, 1H), 3.87 |
| | | | (d, J = 10.7 Hz, 1H), 3.79 (dd, J = 9.8, 5.4 Hz, 1H), |
| | | | 3.57 (s, 1H), 3.49-3.45 (m, 1H), 2.84 (s, 1H), 2.48 |
| | | | (d, J = 1.5 Hz, 3H), 2.37 (s, 1H), 2.15 (ddd, J = |
| | | | 15.1, 13.4, 5.5 Hz, 2H), 1.94 (dd, J = 38.3, 24.0 Hz, |
| | | | 10H), 1.74 (s, 1H), 1.61 (s, 6H), 1.51 (d, J = 12.5 |
| | | | Hz, 2H), 1.11 (d, J = 10.5 Hz, 2H), 1.03 (d, J = |
| | | | 10.7 Hz, 6H), 0.99 (s, 3H). |
| B77 | 958.4 | 958.4 | ¹H NMR (400 MHz, CD3OD) δ 8.86 (d, J = 8.6 |
| | | | Hz, 1H), 8.43 (d, J = 8.4 Hz, 1H), 7.97 (s, 1H), |
| | | | 7.95-7.84 (m, 1H), 7.67 (d, J = 7.2 Hz, 1H), 7.65- |
| | | | 7.56 (m, 1H), 7.55-7.29 (m, 5H), 5.01 (q, J = 7.2 |
| | | | Hz, 1H), 4.73 (t, J = 4.4 Hz, 1H), 4.59 (d, J = 7.6 |
| | | | Hz, 1H), 4.45 (s, 1H), 3.97-3.66 (m, 2H), 3.50 (dd, |
| | | | J = 50.4, 22.4 Hz, 2H), 3.13 (s, 1H), 2.93 (s, 2H), |
| | | | 2.77-2.61 (m, 1H), 2.46 (d, J = 11.2 Hz, 3H), 2.36- |
| | | | 2.16 (m, 1H), 1.97 (ddd, J = 17.5, 13.6, 12.8 Hz, |
| | | | 7H), 1.92-1.67 (m, 7H), 1.62 (s, 2H), 1.59 (s, 6H), |
| | | | 1.51 (d, J = 7.2 Hz, 3H), 1.03 (d, J = 8.8 Hz, 9H). |
| B88 | 930.5 | 930.6 | ¹H NMR (400 MHz, CD3OD) δ8.30-8.25 (m, 1H), |
| | | | 7.98 (d, J = 8.4 Hz, 1H), 7.84 (s, 1H), 7.72 (t, J = |
| | | | 8.0 Hz, 1H), 7.56-7.51 (m, 3H), 7.46-7.31 (m, 4H), |
| | | | 5.03 (s, 1H), 4.95-4.90 (m, 2H), 4.47 (d, J = 18.4 |
| | | | Hz, 8H), 4.31 (s, 1H), 3.85 (s, 3H), 3.75-3.71 (m, |
| | | | 1H), 3.63 (d, J = 7.6 Hz, 1H), 3.54-3.48 (m, 2H), |
| | | | 2.87 (s, 6H), 2.27-2.23 (m, 1H), 2.01 (t, J = 23.6 |
| | | | Hz, 5H), 1.84-1.74 (m, 7H), 1.48 (s, 6H), 1.42-1.36 |
| | | | (m, 3H), 0.93-0.86 (m, 9H). |

The examples in the table below were prepared according to the same method as Compound B3 using appropriate starting materials.

| | Calcd. | Found | |
|---|---|---|---|
| Compound | (M + H)⁺ | (M + H)⁺ | |
| No. | m/z | m/z | ¹H NMR |
| B21 | 964.4 | 964.5 | ¹H NMR (400 MHz, CD3OD) δ 8.98 (s, 1H), 8.46-8.45 |
| | | | (m, 2H), 7.95 (s, 1H), 7.84-7.82 (m, 1H), 7.64-7.60 (m, |
| | | | 2H), 7.38-7.35 (m, 4H), 7.28-7.26 (m, 2H), 5.14-5.13 |
| | | | (m, 1H), 4.83-4.81 (m, 1H), 4.57-4.55 (m, 2H), 4.30- |
| | | | 4.29 (m, 1H), 3.05-3.03 (m, 2H), 2.76-2.72 (m, 1H), |
| | | | 2.30-2.28 (m, 2H), 2.06-2.05 (m, 4H), 1.81-1.70 (m, |
| | | | 7H), 1.63-1.17 (m, 19H), 0.98 (s, 9H). |
| B84 | 930.4 | 930.1 | ¹H NMR (400 MHz, CD3OD) δ 8.88 (s, 1H), 8.43 (d, J = |
| | | | 8.5 Hz, 1H), 7.99 (s, 1H), 7.85 (t, J = 8.3 Hz, 1H), |
| | | | 7.67-7.61 (m, 2H), 7.49-7.25 (m, 5H), 6.30-6.14 (m, |
| | | | 1H), 4.68-4.50 (m, 4H), 4.48-4.40 (m, 1H), 3.96-3.75 |
| | | | (m, 2H), 3.69-3.53 (m, 3H), 3.18-2.94 (m, 5H), 2.51- |
| | | | 2.47 (m, 3H), 2.30-2.27 (m, 3H), 2.23-1.99 (m, 5H), |
| | | | 1.98-1.74 (m, 5H), 1.59 (s, 6H), 1.55-1.42 (m, 3H), |
| | | | 1.09-1.06 (m, 3H), 0.89-0.81 (m, 3H). |
| B85 | 930.4 | 930.1 | ¹H NMR (400 MHz, CD3OD) δ 8.88 (s, 1H), 8.42 (d, J = |
| | | | 8.6 Hz, 1H), 8.09 (d, J = 8.5 Hz, 1H), 7.85 (t, J = 8.3 |
| | | | Hz, 1H), 7.66 (d, J = 7.9 Hz, 1H), 7.57 (s, 1H), 7.50- |
| | | | 7.35 (m, 5H), 6.24 (d, J = 3.0 Hz, 1H), 4.59-4.57 (m, |
| | | | 4H), 4.47-4.40 (m, 1H), 3.88-3.70 (m, 2H), 3.65-3.42 |
| | | | (m, 3H), 2.97-2.66 (m, 5H), 2.48 (d, J = 1.9 Hz, 3H), |
| | | | 2.31-2.24 (m, 3H), 2.21-1.71 (m, 10H), 1.61 (s, 6H), |
| | | | 1.54-1.42 (m, 3H), 1.09-1.06 (m, 3H), 0.91-0.84 (m, |
| | | | 3H). |

The examples in the table below were prepared according to the same method as Compound B5 using appropriate starting materials.

| | Calcd. | Found | |
|---|---|---|---|
| Compound | (M + H)⁺ | (M + H)⁺ | |
| No. | m/z | m/z | ¹H NMR |
| B11 | 918.4 | 918.4 | ¹H NMR (400 MHz, DMSO) δ 8.99 (s, 1H), 8.41-8.40 |
| | | | (m, 2H), 8.05-8.04 (s, 1H), 7.81-7.80 (m, 1H), 7.67-7.56 |
| | | | (m, 2H), 7.44-7.42 (m, 2H), 7.38-7.36 (m, 3H), 7.10- |
| | | | 6.98 (m, 1H), 6.09 (s, 1H), 4.91-4.90 (m, 1H), 4.38-4.36 |
| | | | (m, 1H), 4.29-4.27 (m, 1H), 4.22-4.20 (m, 2H), 3.00- |
| | | | 2.98 (m, 2H), 2.29-2.25 (m, 5H), 2.03-2.01 (m, 4H), |
| | | | 1.92-1.90 (m, 4H), 1.79-1.77 (m, 6H), 1.52-1.50 (s, 6H), |
| | | | 1.47-1.42 (m, 1H), 1.38-1.36 (m, 3H), 0.97-0.95 (m, |
| | | | 3H), 0.81-0, 76 (d, J = 6.0 Hz, 3H). |
| B12 | 932.4 | 932.5 | ¹H NMR (400 MHz, DMSO) δ 8.98 (s, 1H), 8.44-8.40 |
| | | | (m, 2H), 8.27-8.25 (m, 1H), 8.06 (d, J = 8.6 Hz, 1H), |
| | | | 7.81 (t, J = 8.2 Hz, 1H), 7.65-7.58 (m, 2H), 7.48-7.40 |
| | | | (m, 2H), 7.36-7.34 (m, 3H), 6.09 (s, 1H), 5.06-5.04 (m, |
| | | | 1H), 4.95-4.86 (m, 1H), 4.37-4.35 (m, 1H), 4.28-4.26 |
| | | | (m, 1H), 4.18 (t, J = 6.4 Hz, 2H), 4.03-4.01 (m, 2H), |
| | | | 3.65-3.63 (m, 1H), 3.44-3.42 (m, 1H), 3.98-3.00 (m, |
| | | | 2H), 2.39-2.31 (m, 4H), 2.03-2.00 (m, 4H), 1.81-1.72 |
| | | | (m, 6H), 1.58-1.56 (m, 2H), 1.51 (s, 6H), 1.45 (m, 1H), |
| | | | 1.38-1.36 (m, 3H), 0.96-0.94 (m, 3H), 0.83-0.77 (m, |
| | | | 3H). |
| B26 | 918.4 | 918.7 | ¹H NMR (400 MHz, CD3OD) δ 8.94-8.92 (m, 1H), |
| | | | 8.42-8.40 (m, 1H), 7.99-7.98 (m, 1H), 7.87-7.82 (m, |
| | | | 1H), 7.63-7.60 (m, 2H), 7.45-7.31 (m, 6H), 6.03 (s, 1H), |
| | | | 5.04-5.00 (m, 1H), 4.45-4.42 (m, 4H), 3.88-3.75 (m, |
| | | | 3H), 3.71-3.70 (m, 1H), 3.62-3.60 (m, 1H), 3.43-3.36 |
| | | | (m, 2H), 3.31-3.30 (m, 2H), 3.19-3.17 (m, 3H), 2.47- |
| | | | 2.45 (m, 3H), 2.36-2.18 (m, 6H), 2.00-1.92 (m, 1H), |
| | | | 1.58 (s, 6H), 1.52-1.50 (m, 2H), 1.07-1.05 (m, 3H), |
| | | | 0.90-0.88 (m, 3H). |
| B27 | 932.4 | 932.7 | ¹H NMR (400 MHz, CD3OD) δ 8.91-8.90 (s, 1H), 8.42- |
| | | | 8.40 (m, 1H), 7.99-7.96 (m, 1H), 7.86-7.84 (m, 1H), |
| | | | 7.67-7.61 (m, 2H), 7.47-7.31 (m, 6H), 6.02 (s, 1H), |
| | | | 5.10-4.97 (m, 1H), 4.52-4.50 (m, 2H), 4.30-4.28 (m, |
| | | | 2H), 3.8-3.835 (m, 1H), 3.78-3.61 (m, 4H), 3.54-3.45 |
| | | | (m, 1H), 3.15-3.13 (m, 3H), 2.47 (s, 3H), 2.09-2.06 (m, |
| | | | 6H), 1.97-1.95 (m, 6H), 1.59 (s, 6H), 1.54-1.50 (m, 2H), |
| | | | 1.08-1.05 (m, 3H), 0.90-0.88 (m, 3H). |
| B32 | 904.4 | 904.3 | ¹H NMR (400 MHz, CD3OD) δ 8.87 (s, 1H), 8.43 (d, J = |
| | | | 8.6 Hz, 1H), 7.98 (s, 1H), 7.87 (t, J = 8.3 Hz, 1H), |
| | | | 7.67 (d, J = 8.0 Hz, 1H), 7.59 (t, J = 6.2 Hz, 1H), 7.45- |
| | | | 7.32 (m, 5H), 6.05 (s, 1H), 5.02-5.00 (m, 1H), 4.52-4.50 |
| | | | (m, 7H), 3.89-3.81 (m, 1H), 3.71 (d, J = 9.7 Hz, 1H), |
| | | | 3.46 (d, J = 10.0 Hz, 2H), 3.25-3.23 (m, 1H), 2.87-2.84 |
| | | | (m, 3H), 2.46 (s, 3H), 2.38-2.36 (m, 1H), 2.18-2.16 (m, |
| | | | 1H), 2.02-1.90 (m, 5H), 1.59 (s, 6H), 1.52 (d, J = 7.0 |
| | | | Hz, 3H), 1.06 (d, J = 6.6 Hz, 3H), 0.91 (t, J = 7.9 Hz, |
| | | | 3H). |
| B52 | 887.3 | 887.1 | ¹H NMR (400 MHz, CD3OD) δ 8.88-8.84 (m, 1H), |
| | | | 8.42-8.41 (m, 1H), 8.19-8.16 (m, 1H), 8.12-8.09 (m, |
| | | | 1H), 8.02-7.98 (m, 1H), 7.89-7.85 (m, 2H), 7.69-7.68 |
| | | | (m, 2H), 7.45-7.41 (m, 2H), 7.32-7.25 (m, 2H), 6.02- |
| | | | 5.89 (m, 1H), 5.05-4.92 (m, 1H), 4.65-4.51 (m, 6H), |
| | | | 4.44-4.40 (m, 1H), 3.84-3.70 (m, 1H), 3.69-3.44 (m, |
| | | | 2H), 2.48-2.37 (m, 3H), 2.36-2.34 (m, 1H), 2.21-2.11 |
| | | | (m, 1H), 1.98-1.87 (m, 1H), 1.65 (s, 3H), 1.64-1.59 (m, |
| | | | 3H), 1.54-1.50 (m, 1H), 1.44-1.40 (m, 1H), 1.03-1.02 |
| | | | (m, 3H), 0.89-0.86 (m, 3H). |
| B60 | 887.3 | 887.4 | ¹H NMR (400 MHz, CD3OD) δ 8.88-8.85 (m, 1H), |
| | | | 8.59-8.45 (m, 2H), 8.32-8.29 (m, 1H), 8.20 (s, 1H), |
| | | | 8.05-8.02 (m, 1H), 7.93-7.84 (m, 1H), 7.69-7.58 (m, |
| | | | 3H), 7.43-7.39 (m, 4H), 5.97-5.95 (m, 1H), 5.00-4.98 |
| | | | (m, 1H), 3.83-3.81 (m, 1H), 3.66-3.64 (m, 1H), 3.59- |
| | | | 3.57 (m, 1H), 3.49-3.42 (m, 1H), 3.13 (s, 2H), 2.48-2.45 |
| | | | (m 3H), 2.19-2.15 (m, 1H), 1.99-1.91 (m, 1H), 1.62 (s, |
| | | | 4H), 1.64-1.59 (m, 2H), 1.54-1.52 (m, 1H), 1.51-1.49 |
| | | | (m, 2H), 1.29 (s, 1H), 1.17-1.15 (m, 1H), 1.04-1.01 (m, |
| | | | 3H), 0.89-0.85 (m, 4H). |
| B71 | 901.3 | 901.1 | ¹H NMR (400 MHz, CD3OD) δ 8.84-8.81 (m, 1H), |
| | | | 8.40-8.36 (m, 1H), 8.11-8.10 (m, 2H), 7.96 (s, 1H), |
| | | | 7.88-7.76 (m, 2H), 7.69-7.64 (m, 2H), 7.42-7.34 (m, |
| | | | 4H), 7.22-7.19 (m, 1H), 5.98-5.96 (m, 1H), 5.03-4.99 |
| | | | (m, 2H), 4.50-4.47 (m, 3H), 4.43-4.35 (m, 4H), 4.26- |
| | | | 4.21 (m, 2H), 3.89-3.84 (m, 1H), 3.66-3.64 (m, 1H), |
| | | | 3.62 (s, 1H), 3.48 (s, 1H), 2.47 (s, 2H), 2.38-2.30 (m, |
| | | | 4H), 2.17 (s, 1H), 1.99-1.95 (m, 1H), 1.59-1.51 (m, 3H), |
| | | | 1.09-1.04 (m, 3H), 0.93-0.87 (m, 3H). |
| B72 | 901.3 | 901.1 | ¹H NMR (400 MHz, CD3OD) δ 8.89-8.85 (m, 1H), |
| | | | 8.60-8.46 (m, 1H), 8.29-8.15 (m, 2H), 8.06-8.04 (m, |
| | | | 1H), 7.89-7.88 (m, 1H), 7.70-7.56 (m, 3H), 7.47-7.24 |
| | | | (m, 5H), 6.04-5.92 (m, 1H), 5.06-5.02 (m, 2H), 4.53- |
| | | | 4.46 (m, 3H), 4.45-4.37 (m, 4H), 4.26-4.22 (m, 2H), |
| | | | 3.85-3.76 (m, 1H), 3.65-3.63 (m, 1H), 3.58-3.54 (m, |
| | | | 1H), 3.50-3.41 (m, 1H), 2.48 (s, 2H), 2.44-2.29 (m, |
| | | | 4H), 2.21-2.12 (m, 1H), 2.04-1.90 (m, 1H), 1.58-1.51 |
| | | | (m, 3H), 1.15-1.02 (m, 3H), 0.89-0.85 (m, 3H). |
| B74 | 888.4 | 888.4 | ¹H NMR (400 MHz, DMSO) δ 9.53 (s, 1H), 8.49-8.47 |
| | | | (d, J = 8 Hz, 1H), 8.40-8.38 (d, J = 8 Hz, 1H), 8.14- |
| | | | 8.12 (d, J = 8 Hz, 1H), 7.99-7.97 (m, 1H), 7.85-7.80 (m, |
| | | | 1H), 7.75-7.69 (m, 2H), 7.64-7.63 (d, J = 4 Hz, 1H), |
| | | | 7.56 (s, 1H), 7.41-7.33 (m, 3H), 6.20 (s, 1H), 4.57 (s, |
| | | | 2H), 4.48-4.44 (m, 1H), 4.27 (s, 1H), 3.95-3.97 (m, 4H), |
| | | | 3.82-3.57 (m, 6H), 3.45-3.42 (m, 2H), 3.22-3.18 (m, |
| | | | 2H), 2.95 (s, 1H), 2.33 (s, 1H), 2.07-1.95 (m, 5H), 1.52 |
| | | | (s, 6H), 1.39-1.37 (d, J = 8 Hz, 3H), 1.00-0.96 (d, J = |
| | | | 16 Hz, 3H), 0.86-0.85 (d, J = 4 Hz, 3H). |
| B75 | 888.4 | 888.4 | ¹H NMR (400 MHz, DMSO) δ 9.53 (s, 1H), 8.49-8.47 |
| | | | (d, J = 8 Hz, 1H), 8.40-8.38 (d, J = 8 Hz, 1H), 8.14- |
| | | | 8.12 (d, J = 8 Hz, 1H), 7.99-7.97 (m, 1H), 7.85-7.80 (m, |
| | | | 1H), 7.75-7.69 (m, 2H), 7.64-7.63 (d, J = 4 Hz, 1H), |
| | | | 7.56 (s, 1H), 7.41-7.33 (m, 3H), 6.20 (s, 1H), 4.57 (s, |
| | | | 2H), 4.48-4.44 (m, 1H), 4.27 (s, 1H), 3.97-3.95 (m, 4H), |
| | | | 3.82-3.57 (m, 6H), 3.45-3.42 (m, 2H), 3.22-3.18 (m, |
| | | | 2H), 2.95 (s, 1H), 2.33 (s, 1H), 2.07-1.95 (m, 5H), 1.52 |
| | | | (s, 6H), 1.38 (d, J = 8.0 Hz, 3H), 0.98 (d, J = 16 Hz, |
| | | | 3H), 0.85 (d, J = 4 Hz, 3H). |
| B82 | 944.4 | 944.4 | ¹H NMR (400 MHz, CD₃OD) δ 8.86 (s, 1H), 8.40 (d, J = |
| | | | 8.4 Hz, 1H), 8.04 (d, J = 8.0 Hz, 1H), 7.82 (d, J = 8.4 |
| | | | Hz, 1H), 7.64 (d, J = 7.2 Hz, 1H), 7.54 (s, 1H), 7.41 (s, |
| | | | 4H), 7.33 (s, 1H), 6.04 (s, 1H), 5.02 (d, J = 6.4 Hz, 1H), |
| | | | 4.51 (m, 1H), 4.43 (s, 1H), 4.13 (s, 2H), 3.84 (d, J = 7.6 |
| | | | Hz, 1H), 3.67 (d, J = 9.1 Hz, 1H), 3.61 (d, J = 10.2 Hz, |
| | | | 1H), 3.19 (s, 2H), 2.65 (s, 1H), 2.46 (d, J = 1.6 Hz, 3H), |
| | | | 2.43 (s, 2H), 2.40 (s, 1H), 2.19 (d, J = 7.2 Hz, 1H), 2.09- |
| | | | 2.03 (m, 1H), 1.95 (s, 1H), 1.59 (s, 6H), 1.54 (s, 2H), |
| | | | 1.50 (d, J = 7.0 Hz, 3H), 1.06 (s, 2H), 1.04-1.02 (m, |
| | | | 1H), 0.92 (d, J = 7.0 Hz, 2H), 0.89 (s, 2H), 0.68 (s, 2H), |
| | | | 0.51 (s, 2H), 0.01 (d, J = 1.9 Hz, 2H). |
| B91 | 944.4 | 944.4 | ¹H NMR (400 MHz, CD3OD) δ 8.86 (s, 1H), 8.43 (s, |
| | | | 1H), 7.95 (s, 1H), 7.87 (d, J = 8.4 Hz, 1H), 7.66 (d, J = |
| | | | 8.0 Hz, 1H), 7.54 (s, 1H), 7.43-7.42 (m, 1H), 7.41 (s, |
| | | | 2H), 7.37-7.34 (m, 2H), 6.05 (s, 1H), 5.01 (s, 1H), 4.56 |
| | | | (s, 2H), 4.44 (s, 1H), 4.16 (s, 2H), 3.86-3.82 (m, 1H), |
| | | | 3.67 (d, J = 9.2 Hz, 1H), 3.59 (s, 1H), 2.47 (s, 3H), 2.43 |
| | | | (s, 1H), 2.15 (s, 1H), 1.91 (s, 2H), 1.59 (s, 6H), 1.55 (d, |
| | | | J = 4.4 Hz, 2H), 1.51 (s, 2H), 1.50 (s, 2H), 1.06 (s, 2H), |
| | | | 1.04-1.02 (m, 2H), 0.92-0.88 (m, 6H), 0.69 (s, 2H), |
| | | | 0.53 (s, 2H), 0.00 (s, 2H). |
| B94 | 932.4 | 932.1 | ¹H NMR (400 MHz, DMSO) δ 8.98 (s, 1H), 8.39-8.47 |
| | | | (m, 2H), 7.98 (s, 1H), 7.83-7.86 (m, 1H), 7.64 (d, J = |
| | | | 8.0 Hz, 2H), 7.44 (d, J = 8.0 Hz, 2H), 7.36 (d, J = 8.0 |
| | | | Hz, 2H), 7.29 (d, J = 7.2 Hz, 1H), 6.12 (s, 1H), 5.10- |
| | | | 5.12 (m, 1H), 4.96-4.88 (m, 1H), 4.36-4.38 (m, 1H), |
| | | | 4.25 (d, J = 34.8 Hz, 3H), 3.74-3.61 (m, 3H), 3.45 (d, J = |
| | | | 10.8 Hz, 4H), 2.45 (d, J = 4.4 Hz, 4H), 2.34-2.17 (m, |
| | | | 4H), 2.02-2.03 (m, 4H), 1.77-1.79 (m, 4H), 1.51 (s, 6H), |
| | | | 1.38 (d, J = 6.8 Hz, 3H), 0.96 (d, J = 6.4 Hz, 4H), 0.79- |
| | | | 0.81 (m, 3H). |
| B95 | 955.4 | 955.4 | ¹H NMR (400 MHz, CD3OD) δ 8.75 (s, 1H), 8.43-8.40 |
| | | | (m, 1H), 8.16-8.12 (m, 2H), 7.94 (s, 1H), 7.82-7.78 (m, |
| | | | 1H), 7.62-7.52 (m, 4H), 7.38-7.25 (m, 4H), 5.88 (s, |
| | | | 1H), 4.88 (s, 1H), 4.46 (s, 1H), 4.10-4.00 (m, 2H), 3.70- |
| | | | 3.65 (m, 1H), 3.57 (s, 1H), 3.38 (s, 1H), 3.03 (s, 1H), |
| | | | 2.39-2.38 (m, 1H), 2.35 (s, 2H), 1.99-1.93 (m, 3H), 1.52 |
| | | | (s, 6H), 1.42-1.37 (m, 3H), 1.29-1.23 (m, 3H), 1.18-1.12 |
| | | | (m, 6H), 1.00-0.95 (m, 3H), 0.85-0.81 (m, 3H). |
| B96 | 955.4 | 955.4 | ¹H NMR (400 MHz, CD3OD) δ 8.76 (s, 1H), 8.44-8.40 |
| | | | (m, 1H), 8.16-8.08 (m, 2H), 7.94 (s, 1H), 7.83-7.76 (m, |
| | | | 1H), 7.62-7.51 (m, 4H), 7.33-7.25 (m, 4H), 5.91-5.77 |
| | | | (m, 1H), 4.96-4.93 (m, 1H), 4.33 (s, 1H), 3.99 (s, 2H), |
| | | | 3.73-3.70 (m, 4H), 3.56-3.50 (m, 1H), 3.38 (s, 1H), 3.03 |
| | | | (s, 1H), 2.37-2.33 (m, 3H), 1.91 (s, 3H), 1.53 (s, 6H), |
| | | | 1.42-1.40 (m, 3H), 1.39-1.36 (m, 3H), 1.14-1.11 (m, |
| | | | 6H), 0.94-0.90 (m, 3H), 0.82-0.78 (m, 3H). |
| B97 | 915.3 | 915.4 | ¹H NMR (400 MHz, CD3OD) δ 8.89-8.85 (m, 1H), |
| | | | 8.59-8.54 (m, 1H), 8.29-8.27 (m, 1H), 8.21 (s, 1H), 8.03 |
| | | | (s, 1H), 7.90-7.85 (m, 1H), 7.72-7.59 (m, 4H), 7.47-7.37 |
| | | | (m, 4H), 5.95-5.90 (m, 1H), 5.02-5.00 (m, 1H), 4.42 (s, |
| | | | 1H), 4.28-4.24 (m, 2H), 4.22-4.18 (m, 2H), 3.49-3.45 |
| | | | (m, 2H), 3.14-3.12 (m, 1H), 2.45-2.40 (m, 3H), 2.37 (s, |
| | | | 1H), 2.12-2.05 (m, 2H), 1.81 (s, 2H), 1.66-1.61 (m, 6H), |
| | | | 1.56-1.52 (m, 3H), 1.29 (s, 2H), 1.06-1.03 (m, 3H), |
| | | | 0.86-0.84 (m, 3H). |
| B98 | 932.4 | 932.4 | ¹H NMR (400 MHz, CD3OD) δ 8.87 (s, 1H), 8.48-8.44 |
| | | | (m, 1H), 8.05-7.99 (m, 1H), 7.83-7.80 (m, 1H), 7.64- |
| | | | 7.60 (m, 2H), 7.43-7.42 (m, 3H), 7.40-7.34 (m, 3H), |
| | | | 6.00-5.97 (m, 1H), 5.03-5.00 (m, 1H), 4.58-4.54 (m, |
| | | | 1H), 4.43 (s, 1H), 4.26-4.20 (m, 1H), 3.84-3.80 (m, 1H), |
| | | | 3.67-3.61 (m, 2H), 3.48 (s, 1H), 3.13 (s, 2H), 2.47-2.44 |
| | | | (m, 5H), 1.71-1.67 (m, 6H), 1.59-1.55 (m, 2H), 1.50- |
| | | | 1.37 (m, 6H), 1.28 (s, 3H), 1.05-0.99 (m, 4H), 0.91-0.86 |
| | | | (m, 6H). |
| B99 | 915.3 | 915.1 | ¹H NMR (400 MHz, CD3OD) δ 8.85-8.83 (m, 1H), 8.43 |
| | | | (s, 1H), 8.18-8.10 (m, 2H), 7.95 (s, 1H), 7.86-7.84 (m, |
| | | | 2H), 7.73 (s, 1H), 7.65 (s, 1H), 7.46-7.39 (m, 2H), 7.35- |
| | | | 7.33 (m, 2H), 6.02-6.00 (m, 1H), 4.42 (s, 1H), 4.28-4.18 |
| | | | (m, 4H), 3.70-3.68 (m, 2H), 3.48 (s, 1H), 3.13 (s, 1H), |
| | | | 2.47 (s, 1H), 2.43 (s, 2H), 2.21-2.15 (m, 1H), 2.04 (s, |
| | | | 2H), 1.79 (s, 2H), 1.67-1.61 (m, 8H), 1.49-1.44 (m, 4H), |
| | | | 1.06-1.02 (m, 3H), 0.89-0.86 (m, 3H). |

The examples in the table below were prepared according to the same method as Compound B6 using appropriate starting materials.

| | Calcd. | Found ( | |
|---|---|---|---|
| Compound | (M + H)⁺ | M + H)⁺ | |
| No. | m/z | m/z | ¹H NMR |
| B50 | 906.4 | 906.5 | ¹H NMR (400 MHz, CD3OD) δ 8.86 (s, 1H), |
| | | | 8.44 (d, J = 8.8 Hz, 1H), 7.96-7.94 (m, 1H), |
| | | | 7.85-7.84 (m, 1H), 7.65-7.63 (m, 1H), 7.57 |
| | | | (d, J = 7.6 Hz, 1H), 7.44-7.38 (m, 5H), 7.34 |
| | | | (d, J = 7.6 Hz, 1H), 5.00-4.99 (m, 1H), 4.64 (s, |
| | | | 1H), 4.56-4.54 (m, 1H), 4.44-4.42 (s, 1H), 3.87- |
| | | | 3.86 (m, 1H), 3.76 (d, J = 6.8 Hz, 1H), 3.21- |
| | | | 3.17 (m, 2H), 2.47-2.45 (m, 3H), 2.35-2.33 (m, |
| | | | 2H), 2.23-2.14 (m, 2H), 1.96-1.94 (m, 2H), |
| | | | 1.90-1.88 (m, 1H), 1.66-1.64 (m, 3H), 1.59 (s, |
| | | | 6H), 1.50 (d, J = 6.9 Hz, 3H), 1.29-1.28 (s, 4H), |
| | | | 1.06 (s, 9H), 1.03-1.01 (m, 2H). |
| B64 | 889.4 | 889.4 | ¹H NMR (400 MHz, CD3OD) δ 8.87 (s, 1H), |
| | | | 8.55-8.54 (d, J = 8.6 Hz, 1H), 8.25 (s, 1H), 8.21 |
| | | | (s, 1H), 8.03 (s, 1H), 7.89-7.88 (d, J = 8.4 Hz, |
| | | | 1H), 7.69-7.67 (d, J = 8.0 Hz, 1H), 7.63 (s, 2H), |
| | | | 7.43-7.38 (m, 4H), 4.41 (s, 2H), 4.25-4.21 (m, |
| | | | 3H), 2.47 (s, 3H), 2.35-2.32 (t, J = 6.4 Hz, 3H), |
| | | | 2.17-2.13 (m, 1H), 1.97-1.89 (m, 4H), 1.62 (s, |
| | | | 6H), 1.57-1.53 (d, J = 4.4 Hz, 2H), 1.48-1.46 |
| | | | (m, 4H), 1.03 (s, 9H) |
| B83 | 875.3 | 875.1 | 1H NMR (400 MHz, CD3OD) δ 8.86 (s, 1H), |
| | | | 8.57-8.55 (d, J = 8.7 Hz, 1H), 8.25-8.23 (t, J = |
| | | | 5.5 Hz, 2H), 8.04 (s, 1H), 7.92-7.88 (m, 1H), |
| | | | 7.69-7.61 (m, 6.6 Hz, 4H), 7.44-7.37 (m, 4H), |
| | | | 7.28-7.27 (m, 1H), 4.97-4.91 (m, 1H), 4.63 (s, |
| | | | 1H), 4.58-4.56 (d, J = 7.9 Hz, 1H), 4.44 (s, 1H), |
| | | | 4.25-4.22 (m, 2H), 3.94-3.91 (d, J = 10.5 Hz, |
| | | | 1H), 3.78-3.74 (m, 1H), 2.46 (s, 3H), 2.35-2.31 |
| | | | (m, 3H), 2.23-2.16 (m, 3H), 1.98-1.95 (m, 1H), |
| | | | 1.62 (s, 6H), 1.45-1.43 (d, J-7.0 Hz, 3H), 1.06 |
| | | | (s, 9H). |
| B87 | 889.4 | 889.4 | ¹H NMR (400 MHz, CD3OD) δ 8.86 (s, 1H), |
| | | | 8.43 (d, J = 8.4 Hz, 1H), 8.18-8.09 (m, 2H), |
| | | | 7.95 (s, 1H), 7.90-7.81 (m, 2H), 7.73 (dd, J = |
| | | | 8.5, 1.6 Hz, 1H), 7.65 (t, J = 7.7 Hz, 2H), 7.40 |
| | | | (t, J = 6.4 Hz, 3H), 7.16 (s, 1H), 5.00-4.95 (m, |
| | | | 1H), 4.65-4.51 (m, 2H), 4.42 (s, 1H), 4.21 (d, J = |
| | | | 7.2 Hz, 2H), 3.87 (d, J = 11.2 Hz, 1H), |
| | | | 3.74(dd, J = 11.1, 4.0 Hz, 1H), 2.46 (s, 3H), |
| | | | 2.35-2.28 (m, 3H), 1.94 (dd, J = 13.5, 8.4 Hz, |
| | | | 3H), 1.65 (s, 6H), 1.48 (t, J = 6.6 Hz, 5H), 1.03 |
| | | | (s, 9H). |

The examples in the table below were prepared according to the same method as Compound B9 using appropriate starting materials.

| | Calcd. | Found | |
|---|---|---|---|
| Compound | (M + H)⁺ | (M + H)⁺ | |
| No. | m/z | m/z | ¹H NMR |
| B15 | 888.4 | 888.4 | ¹H NMR (400 MHz, CD3OD) δ 8.83 (s, 1H), |
| | | | 8.40-8.38 (m, 1H), 8.07-8.05 (m, 1H), 7.85 (t, J = |
| | | | 8.4 Hz, 1H), 7.66 (d, J-8.0 Hz, 1H), 7.56 (d, |
| | | | J = 8.1 Hz, 1H), 7.40-7.36 (m, 5H), 6.35-6.33 |
| | | | (m, 1H), 5.04 (d, J = 7.0 Hz, 2H), 4.62-4.42 (m, |
| | | | 2H), 3.84-3.82 (m, 2H), 3.63-3.61 (m, 1H), |
| | | | 3.49-3.36 (m, 3H), 3.14-3.01 (m, 3H), 2.86-2.84 |
| | | | (m, 1H), 2.66-2.64 (m, 1H), 2.48 (s, 3H), 2.41- |
| | | | 2.40 (m, 1H), 2.17-2.15 (m, 1H), 2.11-1.83 (m, |
| | | | 5H), 1.59 s, 6H), 1.56-1.48 (m, 3H), 1.08 (d, J = |
| | | | 6.7 Hz, 3H), 0.89-0.87 (m, 3H). |
| B62 | 874.3 | 874.1 | ¹H NMR (400 MHz, CD3OD) δ 8.87 (s, 1H), |
| | | | 8.45-8.44 (m, 1H), 7.96 (s, 1H), 7.89-7.86 (m, |
| | | | 1H), 7.68-7.66 (m, 1H), 7.58-7.57 (m, 1H), |
| | | | 7.52-7.24 (m, 6H), 6.44-6.41 (m, 1H), 5.16 (s, |
| | | | 2H), 5.04-5.00 (m, 2H), 4.52 (s, 1H), 4.44 (s, |
| | | | 1H), 3.85-3.83 (m, 2H), 3.74 (s, 2H), 3.64 (s, |
| | | | 1H), 3.57 (s, 1H), 3.48 (s, 1H), 3.13 (s, 2H), |
| | | | 2.80 (s, 1H), 2.48 (s, 3H), 2.39 (s, 2H), 2.19 (s, |
| | | | 1H), 1.59 (s, 6H), 1.53-1.49 (m, 3H), 1.15-1.08 |
| | | | (m, 3H), 0.89-0.83 (m, 3H). |

The examples in the table below were prepared according to the same method as Compound B9 using appropriate starting materials.

| | Calcd. | Found | |
|---|---|---|---|
| Compound | (M + H)⁺ | (M + H)⁺ | |
| No. | m/z | m/z | ¹H NMR |
| B37 | 957.4 | 479.4 | ¹H NMR (400 MHz, CD3OD) δ 8.87 (s, 1H), 8.43 |
| | | (½M + H) | (d, J = 8.6 Hz, 1H), 7.99 (s, 1H), 7.85 (t, J = |
| | | | 8.3 Hz, 1H), 7.68 (dd, J = 17.0, 8.1 Hz, 2H), |
| | | | 7.60 (d, J = 7.8 Hz, 1H), 7.46-7.38 (m, 4H), |
| | | | 7.36 (t, J = 5.7 Hz, 1H), 7.22 (d, J = 8.0 Hz, |
| | | | 1H), 6.09 (s, 1H), 5.03 (dd, J = 14.1, 7.0 Hz, |
| | | | 1H), 4.57-4.55 (m, 2H), 4.51 (t, J = 8.2 Hz, |
| | | | 1H), 4.44-4.42 (m, 1H), 3.84 (dd, J = 10.7, 4.0 |
| | | | Hz, 1H), 3.73 (d, J = 12.2 Hz, 2H), 3.62-3.60 |
| | | | (m, 2H), 3.57-3.47 (m, 2H), 3.02-3.00 (m, 1H), |
| | | | 2.92-2.90 (m, 2H), 2.87-2.85 (m, 3H), 2.47 (s, |
| | | | 3H), 2.35-2.32 (m, 2H), 2.10-2.08 (m, 4H), |
| | | | 2.06-1.92 (m, 2H), 1.88-1.85 (m, 2H), 1.59 (s, |
| | | | 6H), 1.52 (d, J = 7.0 Hz, 2H), 1.37-1.34 (m, |
| | | | 2H), 1.05 (d, J = 6.6 Hz, 3H), 0.90-0.88 (m |
| | | | 3H). |

Compound B16. (2S,4R)-1-((S)-2-(3-((4-(4-chloro-7,7-dimethyl-5-oxo-5,7-dihydroindolo[1,2-a]quinazolin-9-yl)piperidin-1-yl)methyl)benzamido)-3,3-dimethylbutanoyl)-4-hydroxy-N—((S)-1-(4-(4-methylthiazol-5-yl)phenyl)ethyl)pyrrolidine-2-carboxamide

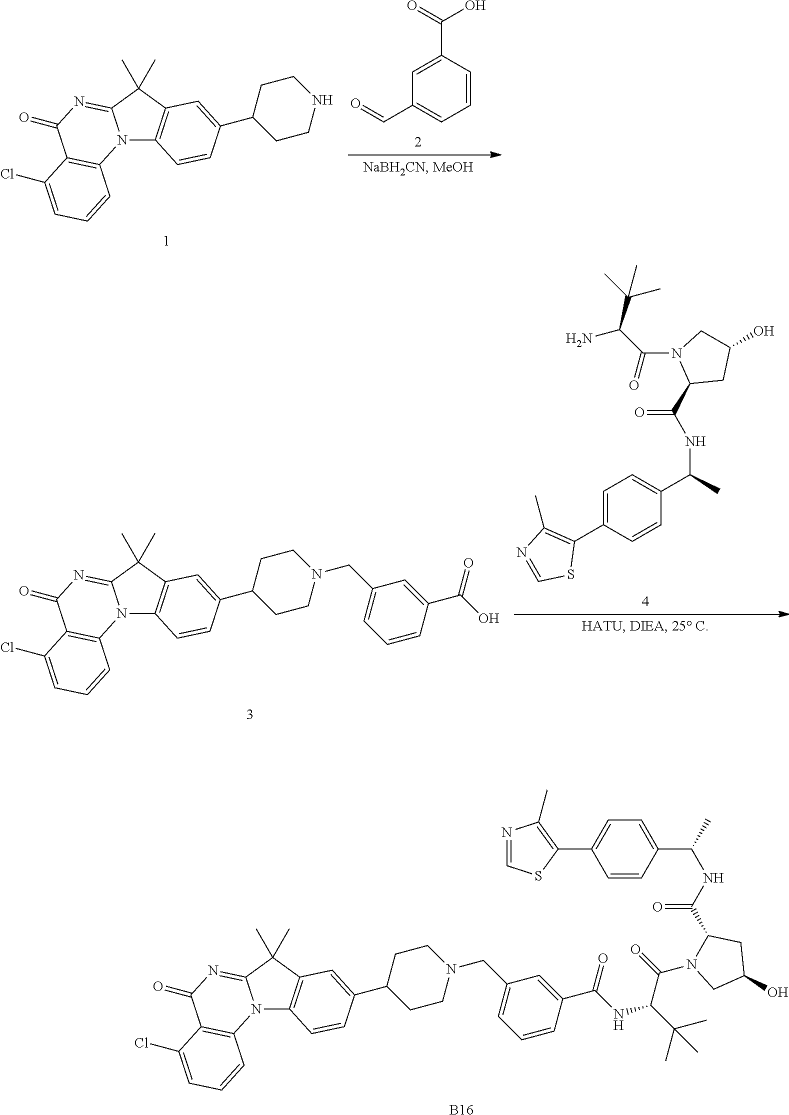

### Synthesis of 3-((4-(4-chloro-7,7-dimethyl-5-oxo-5,7-dihydroindolo[1,2-a]quinazolin-9-yl)piperidin-1-yl)methyl)benzoic acid (Intermediate 3)

To a solution of 4-chloro-7,7-dimethyl-9-(piperidin-4-yl)indolo[1,2-a]quinazolin-5 (7H)-one (100 mg, 1 eq, 0.263 mmol) in MeOH (3 mL) was added 3-formylbenzoic acid (59.3 mg, 1.5 eq, 0.395 mmol), NaBH₃CN (165 mg, 10 eq, 2.63 mmol). The mixture was stirred at 25° C. for 16 h. LCMS indicated completion of reaction. The reaction was poured into H₂O (20 mL) and extracted with DCM (20 mL×3). The combined organics was washed brine (20 mL) and dried over sodium sulfate, filtered and concentrated under vacuum. The resulting residue was purified by prep-TLC (10% of MeOH in DCM) to afford 3-((4-(4-chloro-7,7-dimethyl-5-oxo-5,7-dihydroindolo[1,2-a]quinazolin-9-yl)piperidin-1-yl)methyl)benzoic acid (60 mg, 0.114 mmol, 66.2%) as a yellow oil. LC purity (0.1% FA): 10.9% (UV at 254 nm)/MS: 514.2 [M+1]; Retention time: 1.20 min.

### Synthesis of (2S,4R)-1-((S)-2-(3-((4-(4-chloro-7,7-dimethyl-5-oxo-5,7-dihydroindolo[1,2-a]quinazolin-9-yl)piperidin-1-yl)methyl)benzamido)-3,3-dimethylbutanoyl)-4-hydroxy-N—((S)-1-(4-(4-methylthiazol-5-yl)phenyl)ethyl)pyrrolidine-2-carboxamide (Compound 16)

To a solution of 3-((4-(4-chloro-7,7-dimethyl-5-oxo-5,7-dihydroindolo[1,2-a]quinazolin-9-yl)piperidin-1-yl)methyl)benzoic acid (60 mg, 1 eq, 0.117 mmol) in DMF (2 mL) was added (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (77.8 mg, 1.5 eq, 0.175 mmol), BOP (66.6 mg, 1.5 eq, 0.175 mmol) DIEA (45.3 mg, 3 eq, 0.350 mmol). The mixture was stirred at 65° C. for 16 h. LCMS indicated completion of reaction. The reaction was concentrated under vacuum and purified by prep-HPLC, eluted with MeCN in H2O (0.1% NH₄HCO₃) to afford (2S,4R)-1-((S)-2-(3-((4-(4-chloro-7,7-dimethyl-5-oxo-5,7-dihydroindolo[1,2-a]quinazolin-9-yl)piperidin-1-yl)methyl)benzamido)-3,3-dimethylbutanoyl)-4-hydroxy-N—((S)-1-(4-(4-methylthiazol-5-yl)phenyl)ethyl)pyrrolidine-2-carboxamide (4.32 mg, 0.004 mmol, 3.81%) as a white solid. LC purity (0.03% NH₄HCO₃): 96.47% (UV at 254 nm)/MS: 940.4 [M+1]; Retention time: 1.763 min. ¹H NMR (400 MHz, CD₃OD) δ 8.87 (s, 1H), 8.41-8.48 (m, 1H), 8.05-8.14 (m, 1H), 7.87 (s, 1H), 7.82-7.90 (m, 1H), 7.76-7.82 (m, 1H), 7.64-7.69 (m, 1H), 7.58-7.64 (m, 1H), 7.54 (s, 1H), 7.49-7.54 (m, 1H), 7.43-7.48 (m, 4H), 7.41 (s, 1H), 5.02-5.12 (m 1H), 4.92 (s, 1H), 4.61-4.68 (m, 1H), 4.47 (s, 1H), 3.97-4.02 (m, 1H), 3.82-4.91 (m, 1H), 3.67 (s, 2H), 3.07-3.12 (m, 2H), 2.47 (s, 3H), 2.20-2.28 (m, 4H), 1.97-2.01 (m, 1H), 1.88 (s, 4H), 1.60 (s, 6H), 1.52-1.58 (m, 3H), 1.29 (s, 4H), 1.14 (s, 9H), 1.11 (s, 1H).

The examples in the table below were prepared according to the same method as Compound B16 using appropriate starting materials

| | Calcd. | Found | |
|---|---|---|---|
| Compound | (M + H)⁺ | (M + H)⁺ | |
| No. | m/z | m/z | ¹H NMR |
| B13 | 904.4 | 904.4 | ¹H NMR (400 MHz, DMSO) δ 8.98 (s, 1H), |
| | | | 8.42-8.36 (m, 2H), 8.05-8.03 (m, 1H), 7.81-7.80 |
| | | | (m, 1H), 7.74-7.68 (m, 1H), 7.65-7.62 (m, 2H), |
| | | | 7.44-7.41 (m, 2H), 7.36-7.32 (m, 3H), 5.14-5.09 |
| | | | (m, 1H), 4.92-4.90 (m, 1H), 4.52-4.50 (m, 1H), |
| | | | 4.43-4.41 (m, 1H), 4.31-4.28 (m, 1H), 3.62-3.60 |
| | | | (m, 2H), 2.91-2.88 (m, 4H), 2.69-2.66 (m, 2H), |
| | | | 2.46 (s, 3H), 2.33 (s, 2H), 2.02-1.98 (m, 4H), |
| | | | 1.78-1.75 (m, 5H), 1.52 (s, 6H), 1.37-1.35 (m, |
| | | | 3H), 0.95 (s, 9H) |
| B14 | 904.4 | 904.4 | ¹H NMR (400 MHz, DMSO) δ 8.98 (s, 1H), |
| | | | 8.41-8.37 (m, 2H), 8.07-8.05 (m, 1H), 7.81- |
| | | | 7.79 (m, 1H), 7.75-7.71 (m, 1H), 7.67-7.61 (m, |
| | | | 2H), 7.44-7.42 (m, 2H), 7.39-7.34 (m, 3H), |
| | | | 5.12-5.11 (m, 1H), 4.94-4.89 (m, 1H), 4.55-4.51 |
| | | | (m, 1H), 4.45-4.41 (m, 1H), 4.57-4.29 (m, 1H), |
| | | | 3.61-3.60 (m, 2H), 2.89-2.88 (m, 4H), 2.60-2.58 |
| | | | (m, 2H), 2.46 (s, 3H), 2.01-1.87 (m, 4H), 1.82- |
| | | | 1.75 (m, 5H), 1.72-1.70 (m, 2H), 1.52 (s, 6H), |
| | | | 1.38-1.36 (m, 3H), 0.94 (s, 9H) |
| B20 | 946.4 | 946.4 | ¹H NMR (400 MHz, CD3OD) δ 8.87 (s, 1H), |
| | | | 8.48-8.42 (d, J = 8.4 Hz, 1H), 8.16-8.06 (d, J = |
| | | | 8.0 Hz, 1H), 7.83-7.81 (d, J = 8.0 Hz, 1H), |
| | | | 7.74-7.68 (d, J = 3.6 Hz, 1H), 7.67-7.62 (d, J = |
| | | | 8.0 Hz, 1H), 7.55 (s, 1H), 7.48-7.43 (d, J = 2.4 |
| | | | Hz, 4H), 7.41 (s, 1H), 7.12-7.04 (d, J = 3.6 Hz, |
| | | | 1H), 5.10-5.01 (m, 1H), 4.68-4.60 (m, 1H), |
| | | | 4.46-4.44 (m, 1H), 4.01-3.92 (m, 1H), 3.83-3.80 |
| | | | (m, 2H), 3.18-3.11 (m, 2H), 2.68-2.66 (m, 1H), |
| | | | 2.48 (s, 3H), 2.15-2.10 (m, 4H), 2.03-1.93 (m, |
| | | | 2H), 1.88 (s, 4H), 1.60 (s, 6H), 1.58-1.57 (m, |
| | | | 1H), 1.53 (d, J = 8.0 Hz, 3H), 1.29-1.25 (m, |
| | | | 4H), 1.26-1.24 (m, 1H), 1.11 (s, 9H), 1.08 (s, |
| | | | 2H), 1.00-0.98 (m, 1H). |
| B47 | 932.4 | 932.4 | ¹H NMR (400 MHz, CD3OD) δ 8.87 (s, 1H), |
| | | | 8.43 (d, J = 8.6 Hz, 1H), 7.97 (s, 1H), 7.86 (t, |
| | | | J = 8.3 Hz, 1H), 7.66 (d, J = 7.8 Hz, 1H), 7.58 |
| | | | (d, J = 7.8 Hz, 1H), 7.46-7.40 (m, 4H), 7.3-7.32 |
| | | | (m, 1H), 5.03-4.98 (m, 1H), 4.69-4.55 (m, 2H), |
| | | | 4.44-4.42 (m, 1H), 3.88-3.76 (m, 3H), 2.65 (s, |
| | | | 3H), 2.47 (d, J = 1.4 Hz, 3H), 2.21-2.09 (m, |
| | | | 3H), 2.07-1.89 (m, 7H), 1.88-1.83 (m, 2H), 1.59 |
| | | | (s, 6H), 1.51 (d, J = 6.9 Hz, 4H), 1.20-1.88 (m, |
| | | | 4H), 1.03-1.08 (m, 9H), 0.89-0.85 (m, 1H). |
| B63 | 946.4 | 946.4 | ¹H NMR (400 MHz, CD3OD) δ 8.87 (s, 1H), |
| | | | 8.42 (d, J = 8.0 Hz, 1H), 7.97 (s, 1H), 7.90-7.81 |
| | | | (m, 1H), 7.67 (d, J = 7.9 Hz, 1H), 7.60 (d, J = |
| | | | 7.8 Hz, 1H), 7.46-7.38 (m, 4H), 7.37-7.33 (m, |
| | | | 1H), 5.03-4.98 (m, 1H), 4.67-4.52 (m, 4H), |
| | | | 4.46-4.42 (m, 1H), 3.89 (d, J = 11.3 Hz, 1H), |
| | | | 3.78-3.74 (m, 1H), 3.01-2.80 (m, 4H), 2.46 (s, |
| | | | 3H), 2.22-2.17 m, 3H), 2.16-2.08 (m, 4H), 2.03- |
| | | | 1.90 (m, 5H), 1.88-1.68 (m, 2H), 1.59 (s, 6H), |
| | | | 1.51 (d, J = 7.0 Hz, 4H), 1.22-1.14 (m, 2H), |
| | | | 1.07-1.02 (m, 9H). |

### Compound B29. (2S,4R)-1-((S)-2-(4-((4-(4-chloro-7,7-dimethyl-5-oxo-5,7-dihydroindolo[1,2-a]quinazolin-9-yl)piperidin-1-yl)methyl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide

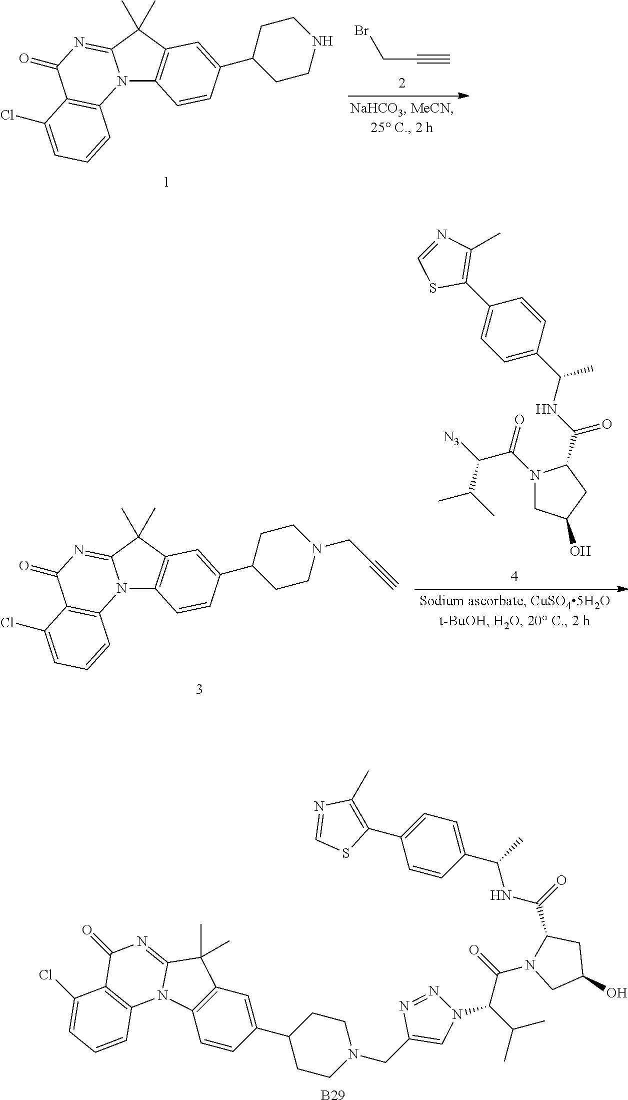

### Synthesis of 4-chloro-7,7-dimethyl-9-(1-(prop-2-yn-1-yl)piperidin-4-yl)indolo[1,2-a]quinazolin-5 (7H)-one (Intermediate 2)

To a solution of 4-chloro-7,7-dimethyl-9-(piperidin-4-yl)indolo[1,2-a]quinazolin-5 (7H)-one (80.0 mg, 1 eq, 211 μmol) in MeCN (3 mL) was added sodium bicarbonate (35.4 mg, 16.4 μL, 2 eq, 421 μmol) and propargyl bromide (20.0 mg, 14.6 μL, 0.8 eq, 168 μmol). The mixture was stirred at 25° C. for 2 hours. LCMS showed the reaction was completed. The reaction was poured into water and extracted with EA, then washed with brine and dried over anhydrous sodium sulfate, filtered and concentrated to get 4-chloro-7,7-dimethyl-9-(1-(prop-2-yn-1-yl)piperidin-4-yl)indolo[1,2-a]quinazolin-5 (7H)-one (100 mg, 0.11 mmol, 51%) as a yellow solid. LC purity (0.1% TFA): 45% (UV at 254 nm)/MS: 418.2 [M+H]; Retention time: 0.97 min.

### Synthesis of (2S,4R)-1-((S)-2-(4-((4-(4-chloro-7,7-dimethyl-5-oxo-5,7-dihydroindolo[1,2-a]quinazolin-9-yl)piperidin-1-yl)methyl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide (compound B29)

To a solution of 4-chloro-7,7-dimethyl-9-(1-(prop-2-yn-1-yl)piperidin-4-yl)indolo[1,2-a]quinazolin-5 (7H)-one (20.0 mg, 1 eq, 47.9 μmol) in t-BuOH (0.50 mL) and water (0.50 mL) was added (2S,4R)-1-((S)-2-azido-3-methylbutanoyl)-4-hydroxy-N—((S)-1-(4-(4-methylthiazol-5-yl)phenyl)ethyl)pyrrolidine-2-carboxamide (26.2 mg, 1.2 eq, 57.4 μmol), copper (II) sulfate pentahydrate (2.39 mg, 0.2 eq, 9.57 μmol) and sodium 2-(1,2-dihydroxyethyl)-4-hydroxy-5-oxo-2,5-dihydrofuran-3-olate (3.79 mg, 0.4 eq, 19.1 μmol). The mixture was stirred at 20° C. for 2 hours. LCMS showed the reaction was completed. The reaction was concentrated under vacuum and purified by prep-HPLC, eluted with MeCN in H₂O (0.1% NH₄HCO₃) to afford (2S,4R)-1-((S)-2-(4-((4-(4-chloro-7,7-dimethyl-5-oxo-5,7-dihydroindolo[1,2-a]quinazolin-9-yl)piperidin-1-yl)methyl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide (2.17 mg, 2.43 μmol, 5.09%) as a white solid. LC purity (0.03% NH₃H₂O): 98.1% (UV at 214 nm)/MS: 874.4 [M+H]; Retention time: 1.60 min. 1H NMR (400 MHz, DMSO) δ 8.99 (s, 1H), 8.50 (d, J=7.6 Hz, 1H), 8.40 (d, J=8.8 Hz, 1H), 8.06 (d, J=8.4 Hz, 2H), 7.82 (t, J=8.4 Hz, 1H), 7.63 (t, J=7.2 Hz, 2H), 7.45 (d, J=8.0 Hz, 2H), 7.37-7.35 (m, 3H), 5.30 (d, J=10.4 Hz, 1H), 5.18 (d, J=3.2 Hz, 1H), 4.93 (t, J=7.2 Hz, 1H), 4.41 (t, J=8.4 Hz, 1H), 4.34-4.32 (m, 1H), 3.79-3.75 (m, 1H), 3.68-3.62 (m, 2H), 3.29-3.27 (m, 2H), 3.10-2.95 (m, 2H), 2.68-2.60 (m, 1H), 2.46 (s, 3H), 2.33-2.31 (m, 1H), 2.13-2.06 (m, 2H), 1.83-1.73 (m, 5H), 1.52 (s, 6H), 1.39 (d, J=7.2 Hz, 3H), 1.07 (d, J=6.4 Hz, 3H), 0.68 (d, J=6.4 Hz, 3H).

The examples in the table below were prepared according to the same method as Compound B29 using appropriate starting materials.

| | Calcd. | Found | |
|---|---|---|---|
| Compound | (M + H)⁺ | (M + H)⁺ | |
| No. | m/z | m/z | ¹H NMR |
| B17 | 888.4 | 888.4 | ¹H NMR (400 MHz, CD3OD) δ 8.88 (s, 1H), |
| | | | 8.42 (d, J = 12 Hz, 1H), 8.09-8.03 (m, 2H), |
| | | | 7.87-7.83 (m, 1H), 7.66-7.64 (d, J = 8 Hz, 1H), |
| | | | 7.57-7.55 (m, 1H), 7.46-7.38 (m, 5H), 5.30 (d, |
| | | | J = 8 Hz, 1H), 5.05-5.02 (m, 2H), 4.58-4.47 (m, |
| | | | 5H), 3.91-3.81 (m, 1H), 3.04-2.91 (m, 4H), |
| | | | 2.78-2.76 (m, 1H), 2.56-2.55 (m, 1H), 2.48-2.44 |
| | | | (m, 4H), 1.61 (s, 6H), 1.53-1.52 (m, 3H), 1.28- |
| | | | 1.25 (m, 2H), 1.14 (d, J = 8 Hz, 3H), 0.78 (d, |
| | | | J = 8 Hz, 3H). |
| B36 | 888.4 | 888.4 | ¹H NMR (400 MHz, CD3OD) δ 8.88 (s, 1H), |
| | | | 8.47-8.45 (d, J = 8.0 Hz, 1H), 8.21-7.99 (d, J = |
| | | | 8 Hz, 2H), 7.89-7.85 (m, 1H), 7.66 (d, J = 8.0 |
| | | | Hz, 1H), 7.59 (d, J = 8.0 Hz, 1H), 7.42-7.37 (m, |
| | | | 5H), 5.30 (d, J = 12 Hz, 1H), 5.04-5.02 (m, |
| | | | 1H), 4.56-4.54 (m, 5H), 3.89-3.84 (m, 2H), |
| | | | 3.01-2.91 (m, 2H), 2.86-2.84 (m, 2 H), 2.48 (ds, |
| | | | 3H), 2.19-2.17 (m, 1H), 2.00-1.98 (m, 4H), 1.59 |
| | | | (s, 6H), 1.51 (d, J = 8.0 Hz, 3H), 1.33-1.29 (m, |
| | | | 4H), 1.14 (d, J = 8.0 Hz, 3H), 0.78 (d, J = 8.0 |
| | | | Hz, 3H). |
| B49 | 874.4 | 874.4 | ¹H NMR (400 MHz, DMSO) δ 8.99 (s, 1H), |
| | | | 8.52-8.49 (m, 2H), 8.42 (d, J = 8.0 Hz , 1H), |
| | | | 7.97 (s, 1H), 7.84 (t, J = 8.0 Hz, 1H), 7.66 (t, |
| | | | J = 8.0 Hz, 2H), 7.46 (d, J = 8.0 Hz 2H), 7.38 (d, |
| | | | J = 8.0 Hz, 2H), 7.25 (d, J = 5.6 Hz, 1H), |
| | | | 5.45(d, J = 9.2 Hz, 1H), 5.24-5.16 (m, 1H), |
| | | | 4.96-4.92 (m, 1H), 4.54-4.52 (m, 2H), 4.40-4.34 |
| | | | (m, 2H), 3.77-3.70 (m, 3H), 3.19-3.03 (m, 4H), |
| | | | 2.47 (s, 3H), 2.15-1.98 (m, 6H), 1.83-1.79 (m, |
| | | | 1H), 1.51 (s, 6H), 1.38 (d, J = 6.8 Hz, 3H), 1.10 |
| | | | (d, J = 6.8 Hz, 3H), 0.73 (d, J = 6.4 Hz, 3H). |
| B68 | 902.4 | 902.5 | ¹H NMR (400 MHz, DMSO) δ 8.99 (s, 1H), |
| | | | 8.68-8.67 (d, J = 2.8 Hz , 1H), 8.53-8.51 (m, |
| | | | 1H), 8.41-8.39 (d, J = 8.8 Hz, 1H), 7.96 (s, |
| | | | 1H), 7.82 (t, J = 8.4 Hz, 1H), 7.68-7.63 (m, |
| | | | 2H), 7.46-7.44 (m, 2H), 7.39-7.36 (m, 2H), |
| | | | 7.25-7.23 (d, J = 8.0 Hz, 1H), 5.64 (s, 1H), |
| | | | 4.94-4.91 (m, 2H), 4.45-4.43 (m, 1H), 4.34 (s, |
| | | | 1H), 3.75-3.65 (m, 6H), 3.00-2.85 (m, 3H), 2.46 |
| | | | (s, 3H), 2.14-2.02 (m, 5H), 1.76-1.74 (d, J = 7.2 |
| | | | Hz, 3H), 1.51 (s, 6H), 1.41-1.39 (d, J = 6.8 Hz, |
| | | | 3H), 1.03 (s, 9H) |
| B69 | 888.4 | 888.4 | ¹H NMR (400 MHz, CD3OD) δ 8.87 (s, 1H), |
| | | | 8.43 (d, J = 8.4 Hz, 1H), 8.32 (s, 1H), 7.95 (s, |
| | | | 1H), 7.88-7.82 (m, 1H), 7.65 (s, 1H), 7.56 (d, |
| | | | J = 7.6 Hz, 1H), 7.44 (d, J = 2.4 Hz, 3H), 7.36 (d, |
| | | | J = 6.4 Hz, 1H), 7.32 (s, 1H), 5.56 (s, 1H), 5.05 |
| | | | (d, J = 6.8 Hz, 1H), 4.56 (m, 2H), 4.45 (s, 1H), |
| | | | 3.85 (s, 1H), 3.78 (s, 2H), 3.34 (d, J = 4.2 Hz, |
| | | | 1H), 3.13 (s, 1H), 3.11-3.08 (m, 1H), 2.48 (s, |
| | | | 3H), 2.39 (s, 1H), 2.20 (d, J = 7.2 Hz, 1H), 1.92 |
| | | | (s, 4H), 1.63 (s, 1H), 1.59 (s, 6H), 1.53 (s, 2H), |
| | | | 1.44 (s, 1H), 1.11 (s, 9H), 0.90 (s, 1H). |

### Compound B78. *2S,4R)-1-((S)-2-(4-((1r,4S)-4-((4-(4-chloro-7,7-dimethyl-5-oxo-5,7-dihydrotodolo[1,2-a]quinazelin-9-yl)piperidin-1-yl)methyl)cyclohexyl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide

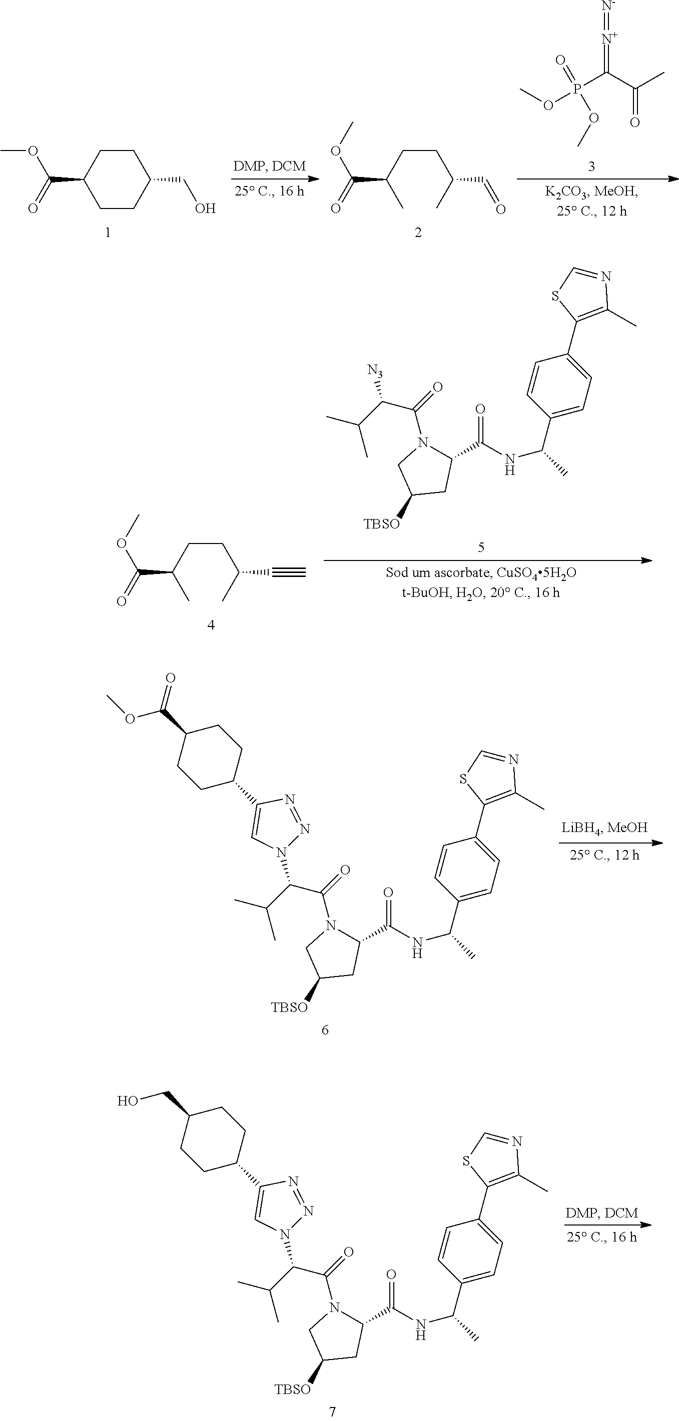

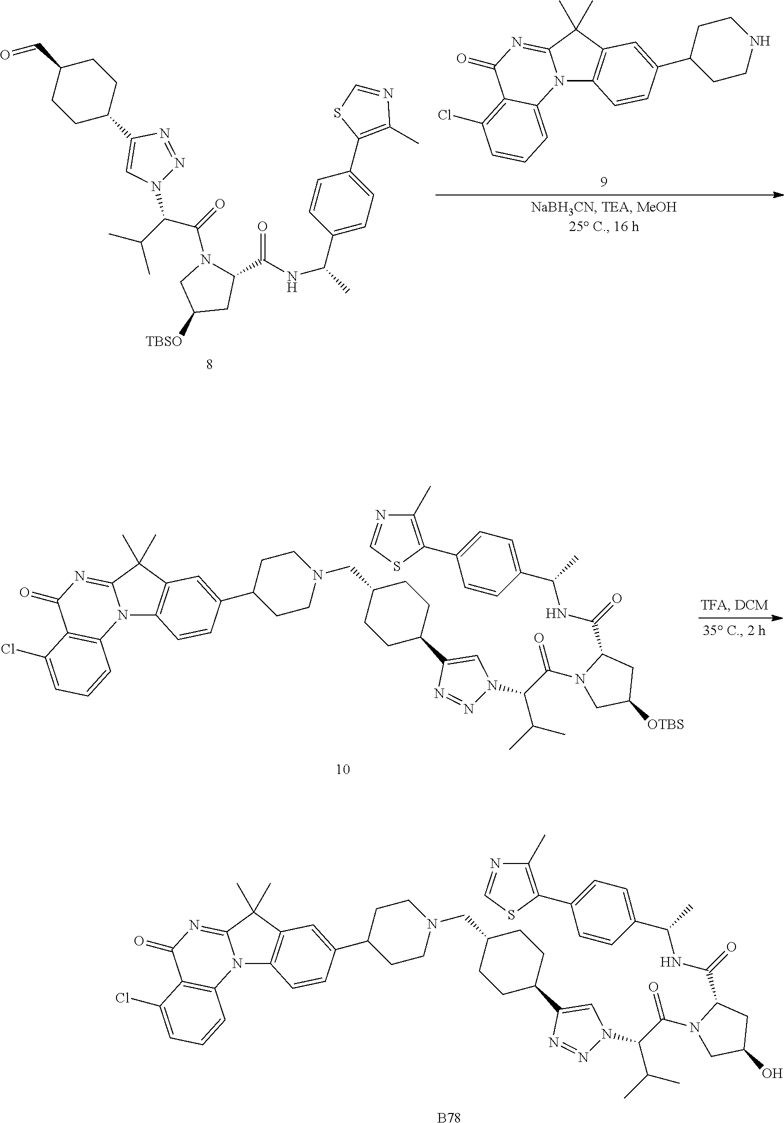

### Synthesis of methyl (1r,4r)-4-formylcyclohexane-1-carboxylate (Intermediate 2)

To a solution of methyl (1r,4r)-4-(hydroxymethyl)cyclohexane-1-carboxylate (2.00 g, 1 eq, 11.6 mmol) in DCM (30 mL) was added DMP (5.91 g, 1.2 eq, 13.9 mmol) at 0° C. The mixture was stirred at 25° C. for 1 hour. TLC showed the reaction was completed. The reaction was quenched by water, washed with EA, washed with brine, dried over anhydrous sodium sulfate, filtered and concentrated. The crude product was purified (PE/EA=5/1) to get methyl (1r,4r)-4-formylcyclohexane-1-carboxylate (1.60 g, 9.40 mmol, 80.9%) as a colourless oil.

### Synthesis of methyl (1r,4r)-4-ethynylcyclohexane-1-carboxylate (Intermediate 4)

To a solution of methyl (1r,4r)-4-formylcyclohexane-1-carboxylate (1.60 g, 1 eq, 9.40 mmol) in DCM (3 mL) was added dimethyl (1-diazo-2-oxopropyl)phosphonate (1.60 g, 1 eq, 9.40 mmol) at 0° C. The mixture was stirred at 25° C. for 1 hour. TLC showed the reaction was completed. The reaction was quenched by water, washed with EA, washed with brine, dried over anhydrous sodium sulfate, filtered and concentrated. The crude product was purified (PE/EA=5/1) to get methyl (1r,4r)-4-ethynylcyclohexane-1-carboxylate (1.00 g, 6.02 mmol, 64.0%) as a colourless oil.

### Synthesis of methyl (1S,4r)-4-(1-((S)-1-((2S,4R)-4-((tert-butyldimethylsilyl)oxy)-2-(((S)-1-(4-(4-methylthiazol-5-yl)phenyl)ethyl) carbamoyl) pyrrolidin-1-yl)-3-methyl-1-oxobutan-2-yl)-1H-1,2,3-triazol-4-yl)cyclohexane-1-carboxylate (Intermediate 6)

To a solution of (2S,4R)-1-((S)-2-azido-3-methylbutanoyl)-4-((tert-butyldimethylsilyl)oxy)-N—((S)-1-(4-(4-methylthiazol-5-yl)phenyl)ethyl)pyrrolidine-2-carboxamide (229 mg, 1 eq, 401 μmol) in t-BuOH (1 mL) and H₂O (2 mL) was added methyl (1r,4r)-4-ethynylcyclohexane-1-carboxylate (100 mg, 1.5 eq, 602 μmol), sodium 2-(1,2-dihydroxyethyl)-4-hydroxy-5-oxo-2,5-dihydrofuran-3-olate (3.18 mg, 0.04 eq, 16.0 μmol) and copper (II) sulfate pentahydrate (2.00 mg, 0.02 eq, 8.02 μmol). The mixture was stirred at 25° C. for 3 hours. LCMS indicated completion of reaction. The reaction was quenched by water, washed with EA, washed with brine, dried over Anhydrous sodium sulfate, filtered and concentrated. The crude product was purified (PE/EA=3/1) to get methyl (1S,4r)-4-(1-((S)-1-((2S,4R)-4-((tert-butyldimethylsilyl)oxy)-2-(((S)-1-(4-(4-methylthiazol-5-yl)phenyl)ethyl) carbamoyl) pyrrolidin-1-yl)-3-methyl-1-oxobutan-2-yl)-1H-1,2,3-triazol-4-yl)cyclohexane-1-carboxylate (248 mg, 0.30 mmol, 75%) as a white solid, LC purity (0.1% FA): 95.3% (UV at 254 nm)/MS: 737.2 [M+H]; Retention time: 1.809 min.

### Synthesis of (2S,4R)-4-((tert-butyldimethylsilyl)oxy)-1-((S)-2-(4-((1r,4S)-4-(hydroxymethyl)cyclohexyl)-1H-1,2,3-triazol-1-yl)-3-methylbutanoyl)-N—((S)-1-(4-(4-methylthiazol-5-yl)phenyl)ethyl)pyrrolidine-2-carboxamide (Intermediate 7)

To a solution of methyl (1S,4r)-4-(1-((S)-1-((2S,4R)-4-((tert-butyldimethylsilyl)oxy)-2-(((S)-1-(4-(4-methylthiazol-5-yl)phenyl)ethyl) carbamoyl) pyrrolidin-1-yl)-3-methyl-1-oxobutan-2-yl)-1H-1,2,3-triazol-4-yl)cyclohexane-1-carboxylate (100 mg, 1 eq, 136 μmol) in MeOH (3 mL) was added NaBH₄ (30.8 mg, 6 eq, 814 μmol) at 0° C. The mixture was stirred at 60° C. for 3 hours. LCMS indicated completion of reaction. The reaction was quenched by water, washed with EA, washed with brine, dried over anhydrous sodium sulfate, filtered and concentrated. The crude product was purified (PE:EA=1:1) to get (2S,4R)-4-((tert-butyldimethylsilyl)oxy)-1-((S)-2-(4-((1r,4S)-4-(hydroxymethyl)cyclohexyl)-1H-1,2,3-triazol-1-yl)-3-methylbutanoyl)-N—((S)-1-(4-(4-methylthiazol-5-yl)phenyl)ethyl)pyrrolidine-2-carboxamide (70.0 mg, 59 μmol, 44%, 60% Purity) as a white solid. LC purity (0.1% FA): 65.0% (UV at 254 nm)/MS: 709.6 [M+H]; Retention time: 1.896 min.

### Synthesis of (2S,4R)-4-((tert-butyldimethylsilyl)oxy)-1-((S)-2-(4-((1r,4S)-4-formylcyclohexyl)-1H-1,2,3-triazol-1-yl)-3-methylbutanoyl)-N—((S)-1-(4-(4-methylthiazol-5-yl)phenyl)ethyl)pyrrolidine-2-carboxamide (Intermediate 8)

To a solution of (2S,4R)-4-((tert-butyldimethylsilyl)oxy)-1-((S)-2-(4-((1r,4S)-4-(hydroxymethyl)cyclohexyl)-1H-1,2,3-triazol-1-yl)-3-methylbutanoyl)-N—((S)-1-(4-(4-methylthiazol-5-yl)phenyl)ethyl)pyrrolidine-2-carboxamide (70.0 mg, 1 eq, 98.7 μmol) in DCM (2 mL) was added DMP (62.8 mg, 1.5 eq, 148 μmol) at 0° C. The mixture was stirred at 25° C. for 2 hours. LCMS indicated completion of reaction. The mixture was filtered and concentrated under vacuum. The mixture was concentrated under vacuum and the residue was purified by silica gel chromatography (100-200 mesh silica gel, 6% of MeOH in DCM) to afford (2S,4R)-4-((tert-butyldimethylsilyl)oxy)-1-((S)-2-(4-((1r,4S)-4-formylcyclohexyl)-1H-1,2,3-triazol-1-yl)-3-methylbutanoyl)-N—((S)-1-(4-(4-methylthiazol-5-yl)phenyl)ethyl)pyrrolidine-2-carboxamide (45.0 mg, 57 μmol, 58%, 90% Purity) as a white oil. LC purity (0.1% FA): 100.00% (UV at 254 nm)/MS: 707.4 [M+H]; Retention time: 1.771 min.

### Synthesis of (2S,4R)-4-((tert-butyldimethylsilyl)oxy)-1-((S)-2-(4-((1r,4S)-4-((4-(4-chloro-7,7-dimethyl-5-oxo-5,7-dihydroindolo[1,2-a]quinazolin-9-yl)piperidin-1-yl)methyl)cyclohexyl)-1H-1,2,3-triazol-1-yl)-3-methylbutanoyl)-N—((S)-1-(4-(4-methylthiazol-5-yl)phenyl)ethyl)pyrrolidine-2-carboxamide (Intermediate 10)

To a solution of (2S,4R)-4-((tert-butyldimethylsilyl)oxy)-1-((S)-2-(4-((1r,4S)-4-formylcyclohexyl)-1H-1,2,3-triazol-1-yl)-3-methylbutanoyl)-N—((S)-1-(4-(4-methylthiazol-5-yl)phenyl)ethyl)pyrrolidine-2-carboxamide (40 mg, 1 eq, 56.6 μmol) in MeOH (1 mL) was added TEA (17.2 mg, 23.7 μL, 3 eq, 170 μmol), 4-chloro-7,7-dimethyl-9-(piperidin-4-yl)indolo[1,2-a]quinazolin-5 (7H)-one (21.5 mg, 1 eq, 56.6 μmol) and NaCNBH₃ (10.7 mg, 3 eq, 170 μmol). The mixture was stirred at 25° C. for 1 hour. LCMS indicated completion of reaction. The mixture was poured into water (20 mL), extracted with ethyl acetate (20 mL×2). The combined organic layers were washed with brine (50 mL), dried over sodium sulfate, filtered and concentrated under vacuum. The mixture was concentrated under vacuum and the residue was purified by silica gel chromatography (100-200 mesh silica gel, 4% of MeOH in DCM) to afford (2S,4R)-4-((tert-butyldimethylsilyl)oxy)-1-((S)-2-(4-((1r,4S)-4-((4-(4-chloro-7,7-dimethyl-5-oxo-5,7-dihydroindolo[1,2-a]quinazolin-9-yl)piperidin-1-yl)methyl)cyclohexyl)-1H-1,2,3-triazol-1-yl)-3-methylbutanoyl)-N—((S)-1-(4-(4-methylthiazol-5-yl)phenyl)ethyl)pyrrolidine-2-carboxamide (50.0 mg, 47 μmol, 83%, 90% Purity) as a white solid. LC purity (0.1% FA): 72.00% (UV at 254 nm)/MS: 536.04 [M+H]; Retention time: 1.535 min.

### Synthesis of (2S,4R)-1-((S)-2-(4-((1r,4S)-4-((4-(4-chloro-7,7-dimethyl-5-oxo-5,7-dihydroindolo[1,2-a]quinazolin-9-yl)piperidin-1-yl)methyl)cyclohexyl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide (Compound B78)

To a solution of (2S,4R)-4-((tert-butyldimethylsilyl)oxy)-1-((S)-2-(4-((1r,4S)-4-((4-(4-chloro-7,7-dimethyl-5-oxo-5,7-dihydroindolo[1,2-a]quinazolin-9-yl)piperidin-1-yl)methyl)cyclohexyl)-1H-1,2,3-triazol-1-yl)-3-methylbutanoyl)-N—((S)-1-(4-(4-methylthiazol-5-yl)phenyl)ethyl)pyrrolidine-2-carboxamide (20.0 mg, 1 eq, 18.7 μmol) in DCM (1 mL) was added TFA (4.44 g, 3.00 mL, 38.9 mmol). The mixture was stirred at 25° C. for 1 hour under N₂. LCMS indicated completion of reaction. The mixture was concentrated under vacuum and the residue was purified by prep-HPLC, eluted with CH₃CN in H₂O from 10% to 95%, (0.1% FA) to afford (2S,4R)-1-((S)-2-(4-((1r,4S)-4-((4-(4-chloro-7,7-dimethyl-5-oxo-5,7-dihydroindolo[1,2-a]quinazolin-9-yl)piperidin-1-yl)methyl)cyclohexyl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide (5.24 mg, 5.25 μmol, 28.1%) as a white solid. LC purity (0.1% FA): 100.00% (UV at 254 nm)/MS: 956.4 [M+H]; Retention time: 1.323 min. ¹H NMR (400 MHz, CD₃OD) δ 8.87 (s, 1H), 8.42 (d, J=8.6 Hz, 1H), 8.10 (d, J=8.5 Hz, 1H), 7.92-7.80 (m, 2H), 7.66 (d, J=7.9 Hz, 1H), 7.58 (s, 1H), 7.49-7.34 (m, 5H), 5.27 (d, J=10.3 Hz, 1H), 5.11-5.01 (m, 1H), 4.64-4.44 (m, 4H), 3.92-3.82 (m, 2H), 3.61-3.44 (m, 2H), 3.01-2.68 (m, 6H), 2.60-2.51 (m, 1H), 2.48 (s, 3H), 2.26-1.91 (m, 10H), 1.62 (s, 6H), 1.59-1.55 (m, 1H), 1.53 (d, J=7.0 Hz, 3H), 1.29-1.12 (m, 2H), 1.13 (d, J=6.5 Hz, 3H), 0.75 (d, J=6.6 Hz, 3H).

The examples in the table below were prepared according to the same method as Compound B78 using appropriate starting materials

| | Calcd. | Found | |
|---|---|---|---|
| Compound | (M + H)⁺ | (M + H)⁺ | |
| No. | m/z | m/z | ¹H NMR |
| B19 | 956.4 | 956.4 | ¹H NMR (400 MHz, CD3OD) δ 8.90 (s, 1H), |
| | | | 8.52-8.50 (m, 2H), 8.42 (d, J = 8.4 Hz, 1H), |
| | | | 8.10 (d, J = 8.6 Hz, 1H), 7.93-7.78 (m, 2H), |
| | | | 7.58 (s, 1H), 7.51-7.20 (m, 5H), 5.32 (d, J = 9.5 |
| | | | Hz, 1H), 4.99-4.92 (m, 1H), 4.64-4.51 (m, 4H), |
| | | | 4.40-4.45 (m, 1H), 3.78 (d, J = 11.1 Hz, 1H), |
| | | | 3.64-3.46 (m, 3H), 3.05-2.68 (m, 6H), 2.59-2.53 |
| | | | (m, 1H), 2.49-2.45 (m, 3H), 2.37-2.35 (m, 1H), |
| | | | 2.30-2.21 (m, 1H), 2.17-2.05 (m, 5H), 2.01-1.90 |
| | | | (m, 3H), 1.64 (s, 6H), 1.57-1.53 (m, 1H), 1.52- |
| | | | 1.45 (m, 3H), 1.29-1.21 (m, 2H), 1.15-1.00 (m, |
| | | | 3H), 0.75-0.68 (m, 3H). |
| B41 | 956.4 | 956.6 | ¹H NMR (400 MHz, DMSO) δ 8.99 (s, 1H), |
| | | | 8.48-8.41 (m, 2H), 7.97 (s, 1H), 7.91 (s, 1H), |
| | | | 7.86-7.82 (m, 1H), 7.70-7.64 (m, 2H), 7.45-7.35 |
| | | | (m, 5H), 7.26 (d, J = 8.0 Hz, 1H), 5.22 (d, J = |
| | | | 8.0 Hz, 1H), 4.91-4.90 (m, 1H), 4.37-4.32 (m, |
| | | | 2H), 3.75-3.65 (m, 4H), 3.06-3.00 (m, 5H), |
| | | | 2.67-2.67 (m, 1H), 2.42 (s, 3H), 2.09-2.05 (m, |
| | | | 7H), 1.94-1.79 (m, 5H), 1.52-1.47 (m, 8H), 1.38 |
| | | | (d, J = 8.0 Hz, 3H), 1.20-1.17 (m, 2H), 1.05- |
| | | | 1.02 (m, 3H), 0.64 (d, J = 8.0 Hz, 3H). |
| B51 | 970.4 | 970.7 | ¹H NMR (400 MHz, DMSO) δ 8.91 (s, 1H), |
| | | | 8.63 (d, J = 8.0 Hz, 1H), 8.44-8.42 (m, 2H), |
| | | | 8.05-8.03 (m, 1H), 7.68-7.62 (m, 2H), 7.64-7.35 |
| | | | (m, 5H), 5.51 (s, 1H), 5.045-5.03 (m, 1H), 4.56- |
| | | | 4.52 (m, 1H), 4.45 (s, 1H), 3.84-3.73 (m, 4H), |
| | | | 3.53-3.51 (m, 1H), 3.27-3.13 (m, 4H), 2.79-2.74 |
| | | | (m, 1H), 2.48 (d, J = 3.2 Hz, 3H), 2.27-2.14 (m, |
| | | | 6H), 2.26-2.17 (m, 4H), 2.14-1.93 (m, 8H), |
| | | | 1.65-1.60 (m, 3H), 1.60-1.53 (m, 3H), 1.07 (s, |
| | | | 9H). |

### Compound B31. (2S,4R)—N—((S)-3-((4-(4-(4-chloro-7,7-dimethyl-5-oxo-5,7-dihydroindolo[1,2-a]quinazolin-10-yl)piperidin-1-yl)cyclohexyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide

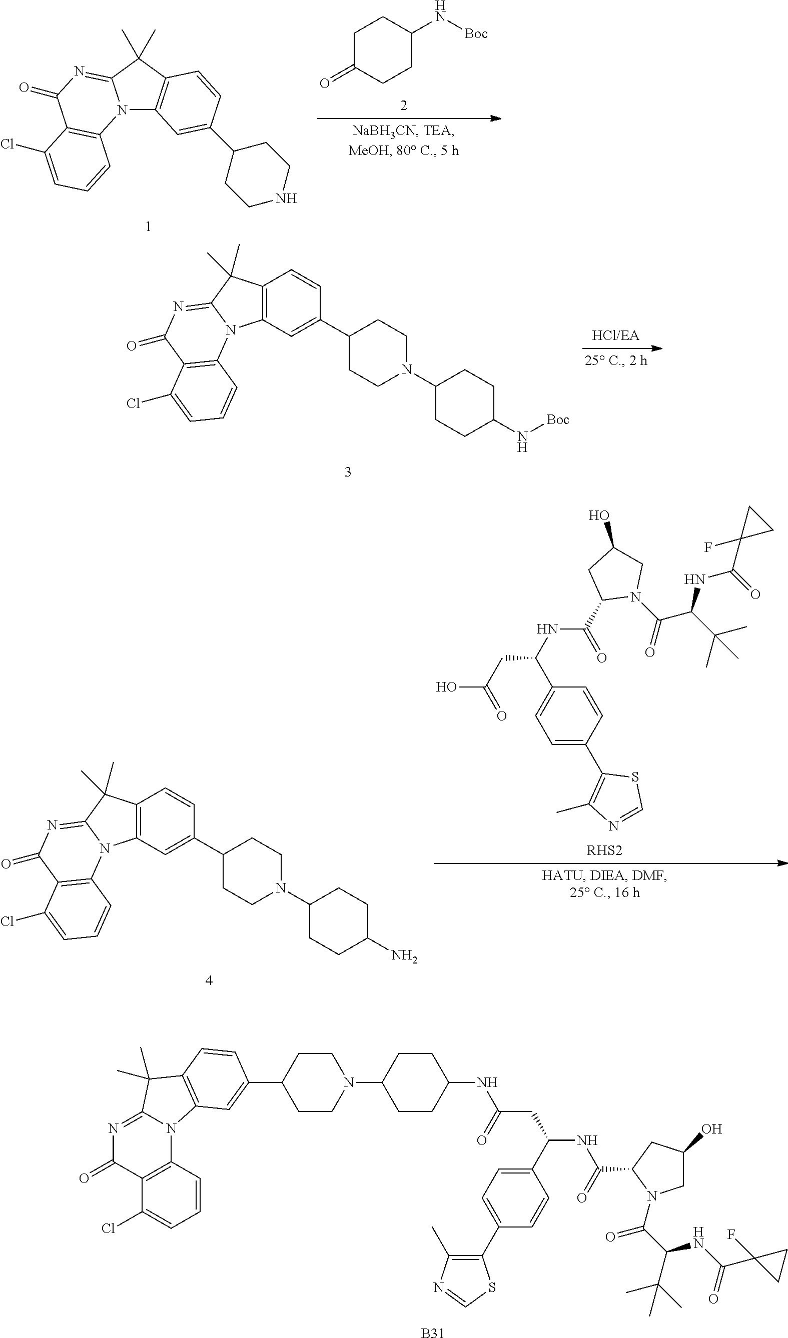

### Synthesis of tert-butyl (4-(4-(4-chloro-7,7-dimethyl-5-oxo-5,7-dihydroindolo[1,2-a]quinazolin-10-yl)piperidin-1-yl)cyclohexyl)carbamate (Intermediate 3)

To a solution of 4-chloro-7,7-dimethyl-10-(piperidin-4-yl)indolo[1,2-a]quinazolin-5 (7H)-one (200 mg, 1 eq, 0.53 mmol) in MeOH (4 mL) was added tert-butyl (4-oxocyclohexyl)carbamate (337 mg, 3 eq, 1.58 mmol) and sodium cyanoborohydride (49.6 mg, 1.5 eq, 0.79 mmol) at room t. The reaction was stirred 80° C. for 5 hours under N₂. LCMS indicated completion of reaction. The mixture was added water, extracted with EA (30 mL×3), dried over anhydrous sodium sulfate and concentrated. The crude product was purified by silica gel column (5% MeOH in DCM) to get tert-butyl (4-(4-(4-chloro-7,7-dimethyl-5-oxo-5,7-dihydroindolo[1,2-a]quinazolin-10-yl)piperidin-1-yl)cyclohexyl)carbamate (99.9 mg, 0.17 mmol, 32.9%) as a yellow oil. LC purity (0.1% FA): 60.0% (UV at 214 nm)/MS: 577.3 [M+H]; Retention time: 0.93 min.

### Synthesis of methyl 10-(1-(4-aminocyclohexyl)piperidin-4-yl)-4-chloro-7,7-dimethylindolo[1,2-a]quinazolin-5 (7H)-one (Intermediate 4)

To a solution of tert-butyl (4-(4-(4-chloro-7,7-dimethyl-5-oxo-5,7-dihydroindolo[1,2-a]quinazolin-10-yl)piperidin-1-yl)cyclohexyl)carbamate (100 mg, 1 e, 0.17 mmol) in Ethyl acetate (2.00 mL) was added HCl in EA (2 mL, 4.00 mmol, 46.2 eq, 8.00 mmol) at room temperature. The reaction was stirred 25° C. for 0.5 hour. LCMS indicated completion of reaction. The mixture was added FA (20 mL) and concentrated, repeated three times to get crude product (70.0 mg, 0.17 mmol, 84.7%). The crude product (15.0 mg) was purified by silica gel prep-HPLC (0.1% FA, eluted with H₂O and CH₃CN from 90:10 to 5:95) to get 10-(1-(4-aminocyclohexyl)piperidin-4-yl)-4-chloro-7,7-dimethylindolo[1,2-a]quinazolin-5 (7H)-one (4.23 mg, 0.009 mmol, 5.10%) as a white solid. LC purity (0.1% TFA): 93.35% (UV at 214 nm)/MS: 477.2 [M+H]; Retention time: 0.88 min.

### Synthesis of tert-butyl (2S,4R)—N—((S)-3-((4-(4-(4-chloro-7,7-dimethyl-5-oxo-5,7-dihydroindolo[1,2-a]quinazolin-10-yl)piperidin-1-yl)cyclohexyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (Compound B31)

To a solution of 10-(1-(4-aminocyclohexyl)piperidin-4-yl)-4-chloro-7,7-dimethylindolo[1,2-a]quinazolin-5 (7H)-one (10 mg, 1 eq, 0.021 mmol) in DMF (1 mL) was added (S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl) propanoic acid (14.5 mg, 1.20 eq, 0.025 mmol), DIEA (13.6 mg, 0.018 mL, 5.00 eq, 0.11 mmol) and HATU (12.0 mg, 1.50 eq, 0.032 mmol) at rt. The reaction was stirred 25° C. for 12 hours under N₂. LCMS indicated completion of reaction. The reaction solution was to cooled and filtered. The crude product was purified by prep-HPLC (0.1% FA, eluted with H₂O:CH₃CN from 90:10 to 5:95) to get (2S,4R)—N—((S)-3-((4-(4-(4-chloro-7,7-dimethyl-5-oxo-5,7-dihydroindolo[1,2-a]quinazolin-10-yl)piperidin-1-yl)cyclohexyl)amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (4.06 mg, 0.004 mmol, 18.1%) as a white solid. LC purity (0.1% FA): 100% (UV at 214 nm)/MS: 1033.7 [M+H]; Retention time: 1.241 min. 19F NMR (376 MHz, DMSO-d₆) δ −196.31. ¹H NMR (400 MHz, DMSO-d₆) δ 8.99 (s, 1H), 8.56-8.62 (d, J=7.2 Hz, 1H), 8.50-8.44 (m, 1H), 7.94-7.82 (m, 2H), 7.64-7.66 (m, 3H), 7.44-7.39 (m, 2H), 7.36 (dd, J=8.4, 3.6 Hz, 2H), 7.25-7.29 (m, 2H), 5.15 (s, 1H), 4.58-4.67 (d, J=9.2 Hz, 1H), 4.47-4.52 (d, J=3.6 Hz, 1H), 4.28 (s, 1H), 3.69 (s, 1H), 3.63-3.53 (m, 3H), 2.92 (s, 2H), 2.62-2.66 (m, 2H), 2.45 (s, 3H), 2.35-2.20 (m, 2H), 2.18-2.01 (m, 3H), 1.76-1.86 (d, J=14.8 Hz, 5H), 1.64-1.77 (d, J=11.2 Hz, 2H), 1.50-1.56 (d, J=4.4 Hz, 6H), 1.43-1.18 (m, 9H), 1.13-1.16 (d, J=6.4 Hz, 1H), 0.97 (s, J=8.0 Hz, 9H).

The examples in the table below were prepared according to the same method as Compound B31 using appropriate starting materials

| | Calcd. | Found | |
|---|---|---|---|
| Compound | (M + H)⁺ | (M + H)⁺ | |
| No. | m/z | m/z | ¹H NMR |
| B22 | 1047.5 | 1047.7 | ¹H NMR (400 MHz, DMSO) δ 9.00 (s, 1H), |
| | | | 8.56 (d, J = 7.6 Hz, 1H), 8.41 (d, J = 8.3 Hz, |
| | | | 1H), 7.96 (s, 1H), 7.82-7.86 (m, 1H), 7.77 (d, |
| | | | J = 7.7 Hz, 1H), 7.67 (dd, J = 13.1, 7.8 Hz, 2H), |
| | | | 7.39 (dd, J = 22.1, 8.3 Hz, 4H), 7.23-7.26 (m, |
| | | | 2H), 5.14-5.18 (m, 1H), 4.58 (d, J = 9.0 Hz, |
| | | | 1H), 4.46 (t, J = 8.1 Hz, 1H), 4.28-4.27 (m, |
| | | | 1H), 3.58-3.55 (m, 4H), 3.43-3.42 (m, 2H), |
| | | | 2.99-2.96 (m, 4H), 2.56-2.54 (m, 1H), 2.45 (s, |
| | | | 3H), 2.07-2.05 (m, 4H), 1.76-1.74 (m, 5H), |
| | | | 1.61-1.60 (m, 1H), 1.51-1.48 (m, 5H), 1.40-1.33 |
| | | | (m, 2H), 1.23-1.11 m, 3H), 1.02-0.95 (m, 12H). |
| B53 | 1047.5 | 1047.5 | ¹H NMR (400 MHz, DMSO) δ 9.00-8.99 (s, |
| | | | 1H), 8.57 (d, J = 8.0 Hz, 1H), 8.4 (d, J = 8.0 |
| | | | Hz, 1H), 7.94 (s, 1H), 7.87-7.82 (m, 2H), 7.69- |
| | | | 7.63 (m, 2H), 7.43-7.41 (m, 2H), 7.38-7.36 (m, |
| | | | 2H), 7.26-7.21 (m, 2H), 5.32-5.21 (m, 1H), |
| | | | 5.21-5.19 (m, 1H), 4.59-4.56 (m, 1H), 4.45-4.43 |
| | | | (m, 1H), 4.27 (s, 1H), 3.59-3.54 (m, 4H), 3.21- |
| | | | 3.01 (m, 9H), 2.65-2.60 (m, 2H), 2.46-2.40 (m, |
| | | | 1H), 2.15-2.12 (m, 3H), 2.05-1.95 (m, 6H), |
| | | | 1.78-1.74 (m, 3H), 1.62-1.51 (m, 3H), 1.39-1.35 |
| | | | (m, 5H), 0.97 (s, 10H), 0.87-0.82 (m, 2H). |
| B54 | 1047.5 | 1047.5 | ¹H NMR (400 MHz, DMSO) δ 9.02 (s, 1H), |
| | | | 8.40 (d, J = 8 Hz, 1H), 7.94 (s, 1H), 7.9-7.87 |
| | | | (m, 2H), 7.69-7.64 (m, 2H), 7.46-7.44 (m, 2H), |
| | | | 7.38-7.36 (m, 2H), 7.26-7.22 (m, 2H), 5.33-5.31 |
| | | | (m, 1H), 5.21-5.15 (m, 1H), 4.59-4.56 (m, 1H), |
| | | | 4.47-4.43 (m, 1H), 4.27-4.26 (m, 1H), 3.61-3.54 |
| | | | (m, 2H), 3.05-2.91 (m, 5H), 2.74-2.73 (m, 1H), |
| | | | 2.66-2.59 (m, 1H), 2.07-1.9 (m, 12H), 1.77-1.76 |
| | | | (m, 1H), 1.73-1.72 (m, 1H), 1.63-1.56 (m, 4H), |
| | | | 0.98 (s, 10H), 0.88-0.78 (m, 4H). |

### Compound B24. (2S,4R)-1-((S)-2-(4-(3-(4-(4-chloro-7,7-dimethyl-5-oxo-5,7-dihydroindolo[1,2-a]quinazolin-9-yl)piperidin-1-yl) propyl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide

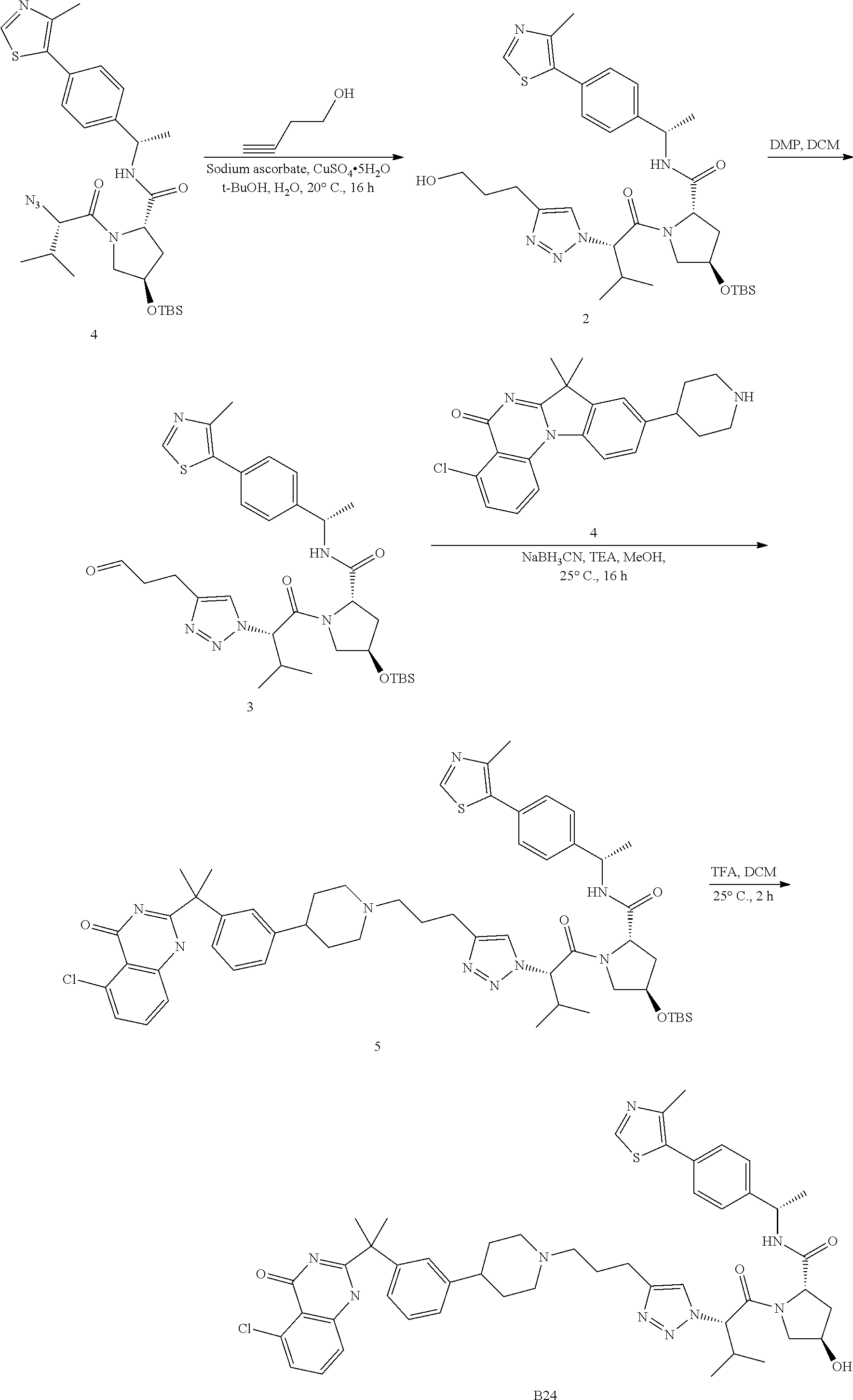

### Synthesis of (2S,4R)-4-((tert-butyldimethylsilyl)oxy)-1-((S)-2-(4-(3-hydroxypropyl)-1H-1,2,3-triazol-1-yl)-3-methylbutanoyl)-N—((S)-1-(4-(4-methylthiazol-5-yl)phenyl)ethyl)pyrrolidine-2-carboxamide (Intermediate 2)

To a solution of (2S,4R)-1-((S)-2-azido-3-methylbutanoyl)-4-((tert-butyldimethylsilyl)oxy)-N—((S)-1-(4-(4-methylthiazol-5-yl)phenyl)ethyl)pyrrolidine-2-carboxamide (100 mg, 1 eq, 0.17 mmol) in Water (1 mL) and t-BuOH (1 mL) was added copper (II) sulfate (2.80 mg, 0.1 eq, 0.017 mmol) and Sodium 2-(1,2-dihydroxy-ethyl)-4-hydroxy-5-oxo-2,5-dihydro-furan-3-olate (17.4 mg, 0.5 eq, 0.087 mmol) and pent-4-yn-1-ol (29.5 mg, 2 eq, 0.35 mmol). The mixture was stirred at 25° C. for 16 hours. LCMS indicated completion of reaction. The reaction was quenched by water (30 ml), washed with EA (20 ml×3), washed with brine (10 ml), dried over anhydrous sodium sulfate, filtered and concentrated. The crude product was purified by silica gel chromatography (100-200 mesh silica gel, 10% of MeOH in DCM) to get (2S,4R)-1-((S)-2-azido-3,3-dimethylbutanoyl)-4-((tert-butyldimethylsilyl)oxy)-N—((R)-1-(4-(4-methylthiazol-5-yl)phenyl)-2-morpholinoethyl)pyrrolidine-2-carboxamide (60.0 mg, 89.6 μmol, 60.5%) as a yellow solid. LC purity (0.03% FA): 90.72% (UV at 254 nm)/MS: 655.4 [M+H]; Retention time: 1.702 min.

### Synthesis of (2S,4R)-4-((tert-butyldimethylsilyl)oxy)-1-((S)-3-methyl-2-(4-(3-oxopropyl)-1H-1,2,3-triazol-1-yl)butanoyl)-N—((S)-1-(4-(4-methylthiazol-5-yl)phenyl)ethyl)pyrrolidine-2-carboxamide (Intermediate 3)

To a solution of (2S,4R)-4-((tert-butyldimethylsilyl)oxy)-1-((S)-2-(4-(3-hydroxypropyl)-1H-1,2,3-triazol-1-yl)-3-methylbutanoyl)-N—((S)-1-(4-(4-methylthiazol-5-yl)phenyl)ethyl)pyrrolidine-2-carboxamide (120 mg, 1 eq, 183 μmol) in DCM (5 mL) was added dess-martinperiodinane (311 mg, 4 eq, 733 μmol). The mixture was stirred at 25° C. for 2 hours. LCMS indicated completion of reaction. The mixture was poured into saturated ammonium chloride solution (20 mL), extracted with DCM (20 mL×2). The combined organic layer was washed with brine (50 mL), dried over sodium sulfate, filtered and concentrated in vacuum. The mixture was concentrated and the residue was purified by column chromatography on silica gel using 0-20% ethyl acetate/hexane to afford (2S,4R)-4-((tert-butyldimethylsilyl)oxy)-1-((S)-3-methyl-2-(4-(3-oxopropyl)-1H-1,2,3-triazol-1-yl)butanoyl)-N—((S)-1-(4-(4-methylthiazol-5-yl)phenyl)ethyl)pyrrolidine-2-carboxamide (70.0 mg, 107 μmol, 58.5%) as a yellow solid. LC purity (0.03% FA): 54.76% (UV at 254 nm)/MS: 653.4 [M+42]; Retention time: 1.745 min.

### Synthesis of (2S,4R)-4-((tert-butyldimethylsilyl)oxy)-1-((S)-2-(4-(3-(4-(4-chloro-7,7-dimethyl-5-oxo-5,7-dihydroindolo[1,2-a]quinazolin-9-yl)piperidin-1-yl) propyl)-1H-1,2,3-triazol-1-yl)-3-methylbutanoyl)-N—((S)-1-(4-(4-methylthiazol-5-yl)phenyl)ethyl)pyrrolidine-2-carboxamide (Intermediate 5)

To a solution of (2S,4R)-4-((tert-butyldimethylsilyl)oxy)-1-((S)-3-methyl-2-(4-(3-oxopropyl)-1H-1,2,3-triazol-1-yl)butanoyl)-N—((S)-1-(4-(4-methylthiazol-5-yl)phenyl)ethyl)pyrrolidine-2-carboxamide (60.0 mg, 1 eq, 91.9 μmol), 4-chloro-7,7-dimethyl-9-(piperidin-4-yl)indolo[1,2-a]quinazolin-5 (7H)-one (52.4 mg, 1.5 eq, 138 μmol) in MeOH (5 mL) was added TEA (46.5 mg, 64.0 μL, 5 eq, 459 μmol). The mixture was stirred at 25° C. for 0.5 hour. Sodium cyanoborohydride (28.9 mg, 27.1 μL, 5 eq, 459 μmol) was added. The reaction was stirred at 25° C. for 16 hours. LCMS indicated completion of reaction. The reaction mixture was diluted with water (20 mL), extracted with DCM (20 mL×3). The combined organic layers were dried over anhydrous sodium sulfate, filtered and concentrated under vacuum. The resulting residue was purified by silica gel column chromatography (100-200 mesh silica gel, 10% of MeOH in DCM) to afford (2_{S},4_{R})-4-((tert-butyldimethylsilyl)oxy)-1-((S)-2-(4-(3-(4-(4-chloro-7,7-dimethyl-5-oxo-5,7-dihydroindolo[1,2-a]quinazolin-9-yl)piperidin-1-yl) propyl)-1_{H}-1,2,3-triazol-1-yl)-3-methylbutanoyl)-_{N}-((_{S})-1-(4-(4-methylthiazol-5-yl)phenyl)ethyl)pyrrolidine-2-carboxamide (50.0 mg, 49.2 μmol, 53.5%) as a yellow oil. LC purity (0.1% NH₄HCO₃): 10.58% (UV at 254 nm)/MS: 1017.6 [M+H]; Retention time: 2.353 min.

### Synthesis of (2S,4R)-1-((S)-2-(4-(3-(4-(4-chloro-7,7-dimethyl-5-oxo-5,7-dihydroindolo[1,2-a]quinazolin-9-yl)piperidin-1-yl) propyl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide (Compound B24)

To a solution of (2S,4R)-4-((tert-butyldimethylsilyl)oxy)-1-((S)-2-(4-(3-(4-(4-chloro-7,7-dimethyl-5-oxo-5,7-dihydroindolo[1,2-a]quinazolin-9-yl)piperidin-1-yl) propyl)-1H-1,2,3-triazol-1-yl)-3-methylbutanoyl)-N—((S)-1-(4-(4-methylthiazol-5-yl)phenyl)ethyl)pyrrolidine-2-carboxamide (70.0 mg, 1 eq, 68.8 μmol) in DCM (2 mL) was added TFA (1.48 g, 1 mL, 189 eq, 13.0 mmol). The mixture was stirred at 25° C. for 2 hours. LCMS indicated completion of reaction. The reaction mixture was concentrated under vacuum. The reaction was purified by prep-HPLC, eluted with MeCN in H₂O from 5% to 95% (0.1% FA) to afford (2S,4R)-1-((S)-2-(4-(3-(4-(4-chloro-7,7-dimethyl-5-oxo-5,7-dihydroindolo[1,2-a]quinazolin-9-yl)piperidin-1-yl) propyl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide (2.79 mg, 2.79 μmol, 4.06%, 90.31% Purity) as a white solid. LC purity (0.1% NH₄·HCO₃): 87.17% (UV at 254 nm)/MS: 902.4 [M+H]; Retention time: 1.892 min. ¹H NMR (400 MHz, CD₃OD) δ 8.84-8.87 (d, J=22.8 Hz, 1H), 8.38-8.42 (m, 1H), 8.08-8.10 (m, 1H), 8.00-8.04 (m, 1H), 7.84-7.86 (m, 1H), 7.62-7.68 (m, 1H), 7.54-7.58 (m, 1H), 7.28-7.48 (m, 5H), 5.32-5.38 (m, 1H), 5.02-5.06 (m, 1H), 4.58-4.66 (m, 1H), 4.48-4.56 (m, 1H), 3.86-3.94 (m, 1H), 3.76-3.86 (m, 1H), 3.64-3.66 (m, 2H), 3.18-3.22 (m, 2H), 3.06-3.08 (m, 2H), 2.86-2.88 (m, 2H), 2.46-2.50 (m, 3H), 2.40 (s, 1H), 2.14-2.20 (m, 6H), 1.98-2.04 (m, 3H), 1.56-1.66 (m, 6H), 1.52-1.58 (m, 3H), 1.12-1.18 (m, 3H), 0.78 (m, 3H).

The examples in the table below were prepared according to the same method as Compound B24 using appropriate starting materials

| | Calcd. | Found | |
|---|---|---|---|
| Compound | (M + H)⁺ | (M + H)⁺ | |
| No. | m/z | m/z | ¹H NMR |
| B58 | 902.4 | 902.4 | ¹H NMR (400 MHz, CD3OD) δ 8.90-8.81 (m, |
| | | | 1H), 8.48-8.36 (m, 1H), 7.98 (d, J = 4.0 Hz, |
| | | | 2H), 7.90-7.82 (m, 1H), 7.68-7.63 (m, 1H), |
| | | | 7.63-7.58 (m, 1H), 7.49-7.39 (m, 4H), 7.36-7.28 |
| | | | (m, 1H), 5.37-5.29 (m, 1H), 5.06-5.00 (m, 1H), |
| | | | 4.62-4.37 (m, 3H), 3.94-3.86 (m, 1H), 3.84-3.74 |
| | | | (m, 1H), 2.92-2.74 (m, 5H), 2.61-2.53 (m, 2H), |
| | | | 2.47 (s, 3H), 2.25-2.17 (m, 1H), 2.10-1.99 (m, |
| | | | 6H), 2.00-1.87 (m, 3H), 1.61-1.57 (m, 6H), |
| | | | 1.58-1.52 (m, 3H), 1.19-1.14 (m, 3H), 0.81-0.77 |
| | | | (m, 3H). |
| B61 | 930.4 | 930.4 | ¹H NMR (400 MHz, CD3OD) δ 8.85 (d, J = |
| | | | 13.2 Hz, 1H), 8.41 (t, J = 10.4 Hz, 1H), 7.97 (d, |
| | | | J = 16.0 Hz, 1H), 7.85 (q, J = 8.0 Hz, 1H), |
| | | | 7.73-7.65 (m, 1H), 7.61 (dd, J = 13.2, 7.8 Hz, |
| | | | 1H), 7.47-7.41 (m, 3H), 7.40-7.20 (m, 2H), 5.30 |
| | | | (t, J = 14.0 Hz, 1H), 5.11-4.96 (m, 1H), 4.62- |
| | | | 4.50 (m, 3H), 4.49 (d, J = 9.6 Hz, 1H), 4.47 (s, |
| | | | 1H), 3.95-3.72 (m, 2H), 3.63 (t, J = 20.4 Hz, |
| | | | 2H), 3.09 (s, 6H), 2.77 (t, J = 7.2 Hz, 2H), 2.46 |
| | | | (d, J = 14.0 Hz, 3H), 2.16 (m, 12.0 Hz, 4H), |
| | | | 2.14-1.89 (m, 4H), 1.80 (dd, J = 15.0, 7.6 Hz, |
| | | | 4H), 1.61 (q, J = 7.2 Hz, 6H), 1.51 (t, J = 9.2 |
| | | | Hz, 3H), 1.51 (t, J = 9.2 Hz, 3H), 1.48 (s, 2H), |
| | | | 1.30 (s, 2H), 1.12 (t, J = 6.0 Hz, 3H), 0.76 (t, |
| | | | J = 6.4 Hz, 3H). |
| B73 | 916.4 | 916.4 | ¹H NMR (400 MHz, CD3OD) δ 8.89-8.85 (m, |
| | | | 1H), 8.60-8.46 (m, 1H), 8.29-8.15 (m, 2H), |
| | | | 8.09-8.04 (m, 1H), 7.92-7.88 (m, 1H), 7.70-7.56 |
| | | | (m, 3H), 7.47-7.24 (m, 5H), 6.04-5.92 (m, 1H), |
| | | | 5.05-5.02 (m, 2H), 4.53-4.46 (m, 3H), 4.45-4.37 |
| | | | (m, 4H), 4.24-4.22 (m, 2H), 3.85-3.76 (m, 1H), |
| | | | 3.69-3.65 (m, 1H), 3.60-3.58 (m, 1H), 3.50- |
| | | | 3.41 (m, 1H), 2.48 (s, 2H), 2.44-2.29 (m, 4H), |
| | | | 2.21-2.12 (m, 1H), 2.04-1.90 (m, 1H), 1.59- |
| | | | 1.51 (m, 3H), 1.05-1.02 (m, 3H), 0.89-0.85 (m, |
| | | | 3H). |

Compound B28. (2S,4R)-1-((R)-2-(3-((4-(4-(4-chloro-7,7-dimethyl-5-oxo-5,7-dihydroindolo[1,2-a]quinazolin-16-yl)piperidin-1-yl)cyclohexyl)oxy)isoxazol-5-7-3-carboxamide

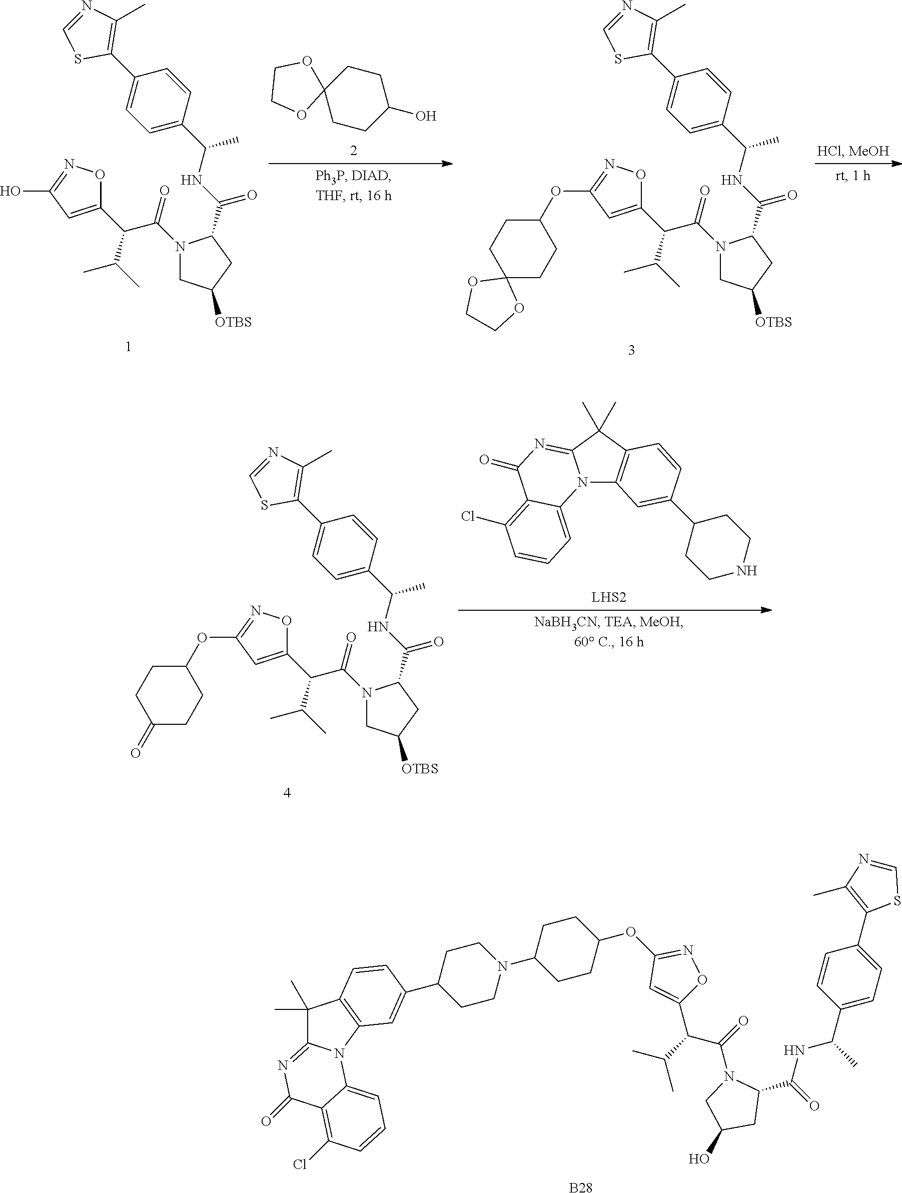

### Synthesis of (2S,4R)-1-((R)-2-(3-((1,4-dioxaspiro[4.5]decan-8-yl)oxy)isoxazol-5-yl)-3-methylbutanoyl)-4-((tert-butyldimethylsilyl)oxy)-N—((S)-1-(4-(4-methylthiazol-5-yl)phenyl)ethyl)pyrrolidine-2-carboxamide (Intermediate 3)

To a solution of (2S,4R)-4-((tert-butyldimethylsilyl)oxy)-1-((R)-2-(3-hydroxyisoxazol-5-yl)-3-methylbutanoyl)-N—((S)-1-(4-(4-methylthiazol-5-yl)phenyl)ethyl)pyrrolidine-2-carboxamide (100 mg, 1 eq, 163 μmol) in THF (2 mL) was added DIAD (99.0 mg, 106 μL, 3.0 eq, 490 μmol), triphenylphosphane (128 mg, 3.0 eq, 490 μmol) and 1,4-dioxaspiro[4.5]decan-8-ol (25.8 mg, 1 eq, 163 μmol) at 0° C. The mixture was stirred at 25° C. for 16 hours under N₂. LCMS indicated completion of reaction. Water (50 mL) was added, extracted with EA (50 mL×2), the organic layer was washed with brine, dried over anhydrous anhydrous sodium sulfate, filtered and concentrated under vacuo. The residue was purified by silica gel column chromatography (100-200 mesh silica gel, 0-10% of MeOH in DCM) to afford (2S,4R)-1-(2-(3-((1,4-dioxaspiro[4.5]decan-8-yl)oxy)isoxazol-5-yl)-3-methylbutanoyl)-4-((tert-butyldimethylsilyl)oxy)-N—((S)-1-(4-(4-methylthiazol-5-yl)phenyl)ethyl)pyrrolidine-2-carboxamide (100.0 mg, 0.11 mmol, 65%, 80% Purity) as a yellow oil. LC purity (0.1% FA): 44.72% (UV at 254 nm)/MS: 753.4 [M+H]; Retention time: 1.78 min.

### Synthesis of (2S,4R)-4-hydroxy-1-((R)-3-methyl-2-(3-((4-oxocyclohexyl)oxy)isoxazol-5-yl)butanoyl)-N—((S)-1-(4-(4-methylthiazol-5-yl)phenyl)ethyl)pyrrolidine-2-carboxamide (Intermediate 4)

To (2S,4R)-1-((S)-2-(3-((1,4-dioxaspiro[4.5]decan-8-yl)oxy)isoxazol-5-yl)-3-methylbutanoyl)-4-((tert-butyldimethylsilyl)oxy)-N—((S)-1-(4-(4-methylthiazol-5-yl)phenyl)ethyl)pyrrolidine-2-carboxamide (70.0 mg, 1 eq, 93.0 μmol) was added HCl (146 mg, 1.00 mL, 4.00 molar, 43.0 eq, 4.00 mmol) at 25° C. The mixture was stirred at 25° C. for 1 hour. LCMS indicated completion of reaction. Water (50 mL) was added, extracted with EA (50 mL×2), the organic layer was washed with brine, dried over anhydrous Anhydrous sodium sulfate, filtered and concentrated under vacuo to get (2S,4R)-4-hydroxy-1-((S)-3-methyl-2-(3-((4-oxocyclohexyl)oxy)isoxazol-5-yl)butanoyl)-N—((S)-1-(4-(4-methylthiazol-5-yl)phenyl)ethyl)pyrrolidine-2-carboxamide (50.0 mg, 76 μmol, 75%, 90% Purity) as a colorless oil. LC purity (0.1% FA): 94.30% (UV at 254 nm)/MS: 595.3 [M+H]; Retention time: 1.45 min.

### Synthesis of (2S,4R)-1-((R)-2-(3-((4-(4-(4-chloro-7,7-dimethyl-5-oxo-5,7-dihydroindolo[1,2-a]quinazolin-10-yl)piperidin-1-yl)cyclohexyl)oxy)isoxazol-5-yl)-3-methylbutanoyl)-4-hydroxy-N—((S)-1-(4-(4-methylthiazol-5-yl)phenyl)ethyl)pyrrolidine-2-carboxamide (Compound 28)

To a solution of (2S,4R)-4-hydroxy-1-((R)-3-methyl-2-(3-((4-oxocyclohexyl)oxy)isoxazol-5-yl)butanoyl)-N—((S)-1-(4-(4-methylthiazol-5-yl)phenyl)ethyl)pyrrolidine-2-carboxamide (31.3 mg, 1 eq, 52.6 μmol) in MeOH (2.00 mL) was added 4-chloro-7,7-dimethyl-10-(piperidin-4-yl)indolo[1,2-a]quinazolin-5 (7H)-one (20.0 mg, 1 eq, 52.6 μmol), TEA (26.6 mg, 36.7 μL, 5 eq, 263 μmol) and Sodium cyanoborohydride (6.62 mg, 2 eq, 105.0 μmol). The mixture was stirred at 25° C. for 16 hours. LCMS indicated completion of reaction. The reaction was quenched by water (10 mL), washed with EA (20 mL), washed with brine, dried over anhydrous sodium sulfate, filtered and concentrated under vacuum. The resulting mixture was purified by prep-HPLC (eluting with H₂O:CH₃CN (0.1% FA) from 90:10 to 5:95) to obtain (2S,4R)-1-((R)-2-(3-((4-(4-(4-chloro-7,7-dimethyl-5-oxo-5,7-dihydroindolo[1,2-a]quinazolin-10-yl)piperidin-1-yl)cyclohexyl)oxy)isoxazol-5-yl)-3-methylbutanoyl)-4-hydroxy-N—((S)-1-(4-(4-methylthiazol-5-yl)phenyl)ethyl)pyrrolidine-2-carboxamide (1.51 mg, 1.51 μmol, 2.87%) as a white solid. LC purity (0.1% FA): 84.65% (UV at 254 nm)/MS: 958.1 [M+H]; Retention time: 1.42 min. ¹H NMR (400 MHz, CD₃OD) δ 8.87 (s, 1H), 8.42 (d, J=8.7 Hz, 1H), 8.10 (d, J=6.8 Hz, 1H), 7.85 (t, J=8.3 Hz, 1H), 7.66 (d, J=7.8 Hz, 1H), 7.56 (s, 1H), 7.50-7.32 (m, 5H), 5.98-5.96 (m, 1H), 5.07-4.98 (m, 1H), 4.59-4.40 (m, 3H), 3.85 (d, J=8.5 Hz, 1H), 3.73-3.57 (m, 2H), 3.51-3.50 (m, 2H), 3.05-3.00 (m, 4H), 2.48 (s, 3H), 2.38-2.34 (m, 3H), 2.14-2.10 (m, 4H), 1.98-1.95 (m, 5H), 1.74-1.72 (m, 2H), 1.61 (8, 6H), 1.60-1.49 (m, 4H), 1.06 (d, J=4.8 Hz, 3H), 0.94-0.87 (m, 3H).

The examples in the table below were prepared according to the same method as Compound B28 using appropriate starting materials

| | Calcd. | Found | |
|---|---|---|---|
| Compound | (M + H)⁺ | (M + H)⁺ | |
| No. | m/z | m/z | ¹H NMR |
| B30 | 972.4 | 486.8 | ¹H NMR (400 MHz, CD₃OD) δ 8.87 (s, 1H), |
| | | | 8.42 (d, J = 8.7 Hz, 1H), 8.11 (d, J = 8.6 Hz, |
| | | | 1H), 7.86-7.85 (m, 1H), 7.66 (d, J = 8.0 Hz, |
| | | | 1H), 7.58 (s, 1H), 7.49-7.37 (m, 5H), 6.08-5.85 |
| | | | (m, 1H), 5.04-5.03 (m, 1H), 4.50 (m, 2H), 3.85- |
| | | | 3.83 (m, 1H), 3.76-3.58 (m, 4H), 3.50-3.48 (m, |
| | | | 1H), 3.57-3.08 (m, 4H), 2.48 (s, 3H), 2.39-2.31 |
| | | | (m, 2H), 2.14-2.10 (m, 6H), 1.98-1.95 (m, 3H), |
| | | | 1.73-1.71 (m, 2H), 1.61 (s, 6H), 1.58-1.47 (m, |
| | | | 5H), 1.25 (d, J = 6.1 Hz, 1H), 1.06 (d, J = 6.4 |
| | | | Hz, 3H), 0.91-0.89 (m, 4H). |
| B38 | 972.4 | 972.6 | ¹H NMR (400 MHz, CD₃OD) δ 8.87 (s, 1H), |
| | | | 8.42 (d, J = 8.4 Hz, 1H), 7.99 (s, 1H), 7.86-7.83 |
| | | | (m, 1H), 7.68-7.61 (m, 2H), 7.45-7.34 (m, 5H), |
| | | | 6.01-5.98 (m, 1H), 5.04-5.02 (m, 1H), 4.51-4.44 |
| | | | (m, 2H), 3.84-3.83 (m, 1H), 3.70 3.62 (m, 4H), |
| | | | 3.13-2.94 (m, 6H), 2.48 (s, 3H), 2.38-2.32 (m, |
| | | | 2H), 2.18-2.16 (m, 6H), 2.02-1.96 (m, 3H), |
| | | | 1.73-1.71 (m, 3H), 1.59 (s, 6H), 1.55-1.51 (m, |
| | | | 4H), 1.23-1.21 (m, 1H), 1.06 (d, J = 6.4 Hz, |
| | | | 3H), 0.90-0.89 (m, 3H). |
| B81 | 958.4 | 958.1 | ¹H NMR (400 MHz, CD₃OD) δ 8.87 (s, 1H), |
| | | | 8.21 (m, 1H), 7.70-7.49 (m, 2H), 7.49-7.11 (m, |
| | | | 6H), 6.99-6.86 (m, 1H), 6.06-5.86 (m, 1H), |
| | | | 5.12 (s, 1H), 5.03 (d, J = 6.1 Hz, 2H), 4.60- |
| | | | 4.39 (m, 3H), 3.85 (d, J = 11.1 Hz, 1H), 3.72- |
| | | | 3.43 (m, 5H), 3.19 (m, 2H), 2.48 (s, 3H), 2.21 |
| | | | (m, 8H), 2.04-1.82 (m, 5H), 1.65-1.49 (m, 6H), |
| | | | 1.44 (d, J = 7.1 Hz, 3H), 1.29 (s, 1H), 1.06 (d, |
| | | | J = 6.5 Hz, 3H), 0.89 (d, J = 4.5 Hz, 3H). |
| B100 | 972.4 | 972.1 | ¹H NMR (400 MHz, CD3OD) δ 8.87 (s, 1H), |
| | | | 8.52 (s, 1H), 8.43 (s, 1H), 8.05-8.03 (d, J = 8.0 |
| | | | Hz, 1H), 7.85-7.83 (m, 1H), 7.67-7.65 (d, J = |
| | | | 8.2 Hz, 1H), 7.56-7.54 (d, J = 8.8 Hz, 1H), |
| | | | 7.46-7.37 (m, 5H), 6.03-6.00 (d, J = 9.6 Hz, |
| | | | 1H), 5.04-5.01 (d, J = 7.2 Hz, 2H), 4.52-4.50 |
| | | | (m, 1H), 4.45 (s, 1H), 4.28-4.24 (d, J = 6.4 Hz, |
| | | | 1H), 4.04 (s, 1H), 3.85-3.82 (d, J = 6.8 Hz, 1H), |
| | | | 3.72-3.64 (m, 2H), 3.49 (s, 2H), 3.12-3.05 (m, |
| | | | 2H), 2.47 (s, 3H), 2.37 (s, 1H), 2.08-2.00 (m, |
| | | | 6H), 1.74 (s, 8H), 1.67 (s, 1H), 1.54-1.50 (d, J = |
| | | | 6.8 Hz, 3H), 1.26-1.22 (d, J = 6.4 Hz, 6H), |
| | | | 1.06-1.02 (d, J = 6.4 Hz, 3H), 0.89-0.86 (d, J = |
| | | | 6.8 Hz, 3H). |

### Compound B33. (2S,4R)—N—((S)-3-((4-(4-(4-chloro-7,7-dimethyl-5-oxo-5,7-dihydroindolo[1,2-a]quinazolin-10-yl)piperidin-1-yl)butyl) amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide

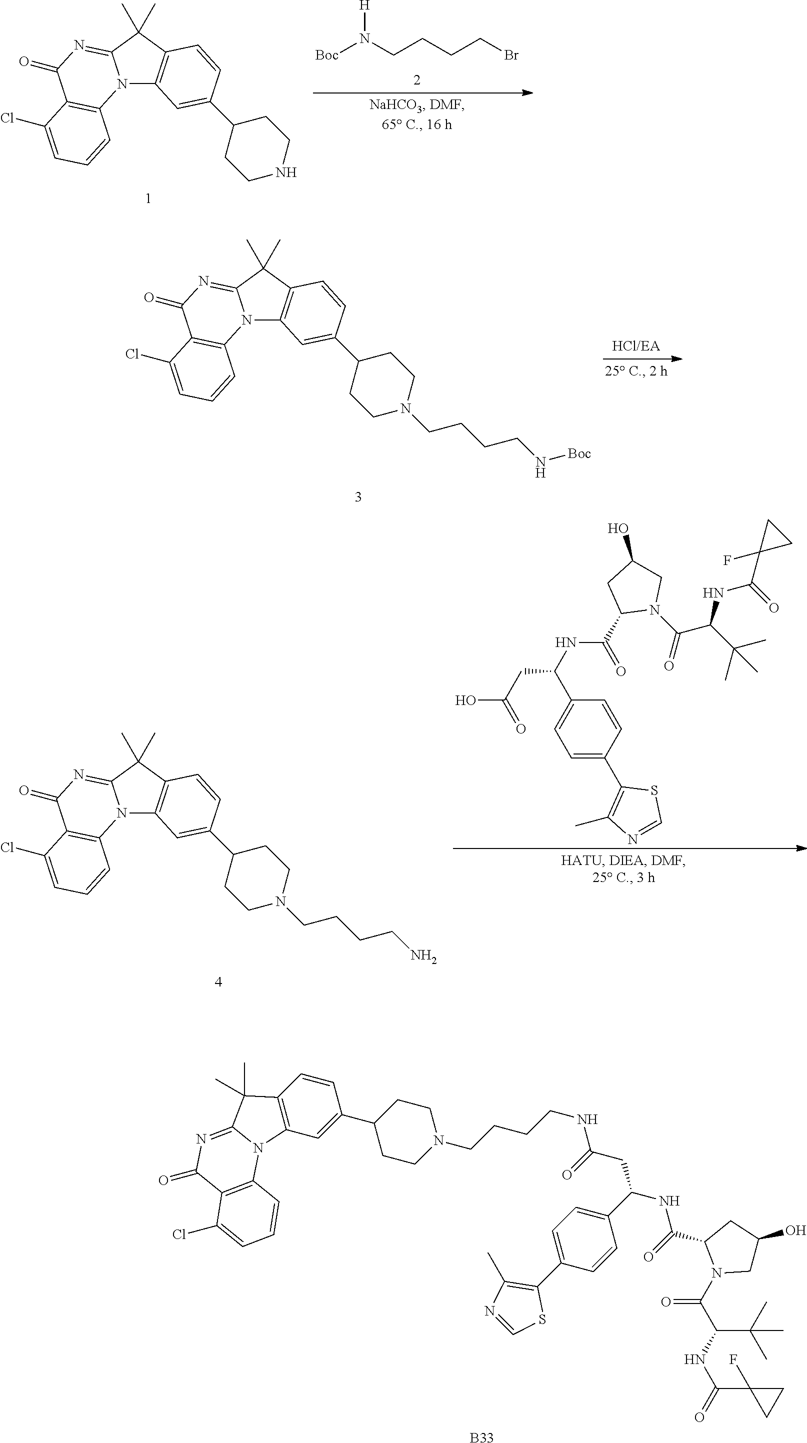

### Synthesis of tert-butyl 4-(3′-(2-hydroxyphenyl)-7′,8′-dihydro-5′H-spiro[piperidine-4,6′-pyrazino[2,3-c]pyridazin]-1-yl)piperidine-1-carboxylate (Intermediate 3)

To a solution of 4-chloro-7,7-dimethyl-10-(piperidin-4-yl)indolo[1,2-a]quinazolin-5 (7H)-one (150 mg, 1 eq, 395 μmol) in DMF (10 mL) was added Sodium bicarbonate (332 mg, 154 μL, 10 eq, 3.95 mmol) and tert-butyl (4-bromobutyl)carbamate (149 mg, 1.5 eq, 592 μmol). The reaction was stirred at 65° C. for 16 hours. LCMS indicated completion of reaction. The mixture was added water (20 mL), extracted with MeOH/DCM=1/10 (30 mL), dried over anhydrous sodium sulfate and concentrated. The crude product was purified by silica prep-TLC (MeOH/DCM=1/10) to get tert-butyl (4-(4-(4-chloro-7,7-dimethyl-5-oxo-5,7-dihydroindolo[1,2-a]quinazolin-10-yl)piperidin-1-yl)butyl)carbamate (160 mg, 290 μmol, 73.5%) as a yellow solid. LC purity (0.03% TFA): 84.81% (UV at 254 nm)/MS: 551.4 [M+H]; Retention time: 0.901 min.

### Synthesis of 10-(1-(4-aminobutyl)piperidin-4-yl)-4-chloro-7,7-dimethylindolo[1,2-a]quinazolin-5 (7H)-one (Intermediate 4)

To a solution of tert-butyl (4-(4-(4-chloro-7,7-dimethyl-5-oxo-5,7-dihydroindolo[1,2-a]quinazolin-10-yl)piperidin-1-yl)butyl)carbamate (10.0 mg, 1 eq, 18.1 μmol) in Hydrogen chloride (1.9 g, 2.00 mL, 52 mmol). The reaction was stirred at 25° C. for 2 hours. LCMS indicated completion of reaction. The reaction was added DCM (10 mL×3) to concentrated to get 10-(1-(4-aminobutyl)piperidin-4-yl)-4-chloro-7,7-dimethylindolo[1,2-a]quinazolin-5 (7H)-one (2.11 mg, 4.59 μmol, 25.3%) as a white solid. LC purity (0.1% FA): 97.51% (UV at 254 nm)/MS: 451.4 [M+H]; Retention time: 0.16 min.

### Synthesis of (2S,4R)—N—((S)-3-((4-(4-(4-chloro-7,7-dimethyl-5-oxo-5,7-dihydroindolo[1,2-a]quinazolin-10-yl)piperidin-1-yl)butyl) amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (Compound B33)

To a solution of 10-(1-(4-aminobutyl)piperidin-4-yl)-4-chloro-7,7-dimethylindolo[1,2-a]quinazolin-5 (7H)-one (30.0 mg, 1 eq, 66.5 μmol) in DMF (2 mL) was added DIEA (43.0 mg, 57.9μ, 5 eq, 333 μmol) and HATU (37.9 mg, 1.5 eq, 99.8 μmol) and(S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl) propanoic acid (45.9 mg, 1.2 eq, 79.8 μmol) LCMS indicated completion of reaction. The mixture was added water (20 mL), extracted with Me:DCM=1:10 (30 mL), dried over anhydrous sodium sulfate and concentrated. The crude product was purified by prep-HPLC (0.1% NH₄HCO₃ in CH₃CN and H₂O) to get (2S,4R)—N—((S)-3-((4-(4-(4-chloro-7,7-dimethyl-5-oxo-5,7-dihydroindolo[1,2-a]quinazolin-10-yl)piperidin-1-yl)butyl) amino)-1-(4-(4-methylthiazol-5-yl)phenyl)-3-oxopropyl)-1-((S)-2-(1-fluorocyclopropane-1-carboxamido)-3,3-dimethylbutanoyl)-4-hydroxypyrrolidine-2-carboxamide (8.31 mg, 7.48 μmol, 11.2%) as a white solid. LC purity (0.1% FA): 100% (UV at 254 nm)/MS: 1007.6 [M+H]; Retention time: 1.40 min. 1H NMR (400 MHz, CD₃OD) δ 8.86 (m, 1H), 8.44 (d, J=8.4 Hz, 1H), 7.94 (s, 1H), 7.86 (t, J=8.4 Hz, 1H), 7.66 (d, J=7.6 Hz, 1H), 7.57 (d, J=7.6 Hz, 1H), 7.48-7.40 (m, 4H), 7.30 (d, J=7.2 Hz, 1H), 5.33-5.30 (m, 1H), 4.73 (s, 1H), 4.58 (t, J=8.4 Hz, 1H), 4.44-4.42 (m, 1H), 3.79-3.75 (m, 2H), 3.20-3.00 (m, 2H), 2.86-2.80 (m, 1H), 2.47 (s, 3H), 2.20-2.18 (m, 3H), 2.00-1.89 (m, 4H), 1.59 (s, 6H), 1.43-1.39 (m, 3H), 1.40-1.14 (m, 5H), 1.10 (s, 9H).

The examples in the table below were prepared according to the same method as Compound B33 using appropriate starting materials

| | Calcd. | Found | |
|---|---|---|---|
| Compound | (M + H)⁺ | (M + H)⁺ | |
| No. | m/z | m/z | ¹H NMR |
| B25 | 993.4 | 498.0 | ¹H NMR (400 MHz, DMSO) δ 8.97 (s, 1H), |
| | | | 8.57-8.55 (m, 1H), 8.47-8.45 (m, 1H), 7.92 (s, |
| | | | 1H), 7.83-7.81 (m, 2H), 7.62-7.60 (m, 2H), |
| | | | 7.44-7.41 (m, 2H), 7.40-7.32 (m, 2H), 7.25 |
| | | | (d, J = 8.0 Hz, 2H), 5.19-5.17 (m, 2H), 4.58- |
| | | | 4.56 (m, 1H), 4.46-4.44 (m, 1H), 4.29-4.27 (m, |
| | | | 1H), 3.59-3.57 (m, 2H), 3.10-3.08 (m, 2H), |
| | | | 2.99-2.93 (m, 1H), 2.89-2.87 (m, 2H), 2.60-2.56 |
| | | | (m, 3H), 2.47 (s, 3H), 2.16-2.14 (m, 2H), 2.09- |
| | | | 2.02 (m, 1H), 1.90-1.88 (m, 2H), 1.72-1.70 (m, |
| | | | 4H), 1.51 (s, 6H), 1.47-1.41 (m, 2H), 1.36-1.34 |
| | | | (m, 2H), 1.22-1.20 (m, 2H), 0.97 (s, 9H). |
| B44 | 979.4 | 979.5 | ¹H NMR (400 MHz, CD₃OD) δ 8.83 (s, 1H), |
| | | | 8.42-8.40 (d, J = 9.2 Hz, 1H), 7.88-7.86 (m, |
| | | | 2H), 7.68-7.65 (m, 1H), 7.56-7.54 (m, 1H), |
| | | | 7.48-7.45 (m, 4H), 7.29-7.26 (m, 1H), 5.35-5.33 |
| | | | (m, 1H), 4.73-4.71 (m, 1H), 4.59-4.57 (m, 1H), |
| | | | 4.44-4.42 (m, 1H), 3.86-3.71 (m, 2H), 3.10-3.08 |
| | | | (d, J = 31.2 Hz, 3H), 2.86-2.84 (m, 1H), 2.77- |
| | | | 2.75 (m, 2H), 2.46-2.45 (m, 4H), 2.16-2.14 (m, |
| | | | 3H), 1.96-1.94 (m, 1H), 1.87-1.85 (m, 5H), 1.59 |
| | | | (s, 6H), 1.35-1.32 (m, 5H), 1.06 (s, 9H). |

### Compound B35. (2S,4R)—N-(2-(4-(4-(4-chloro-7,7-dimethyl-5-oxo-5,7-dihydroindolo[1,2-a]quinazolin-10-yl)piperidin-1-yl)butoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

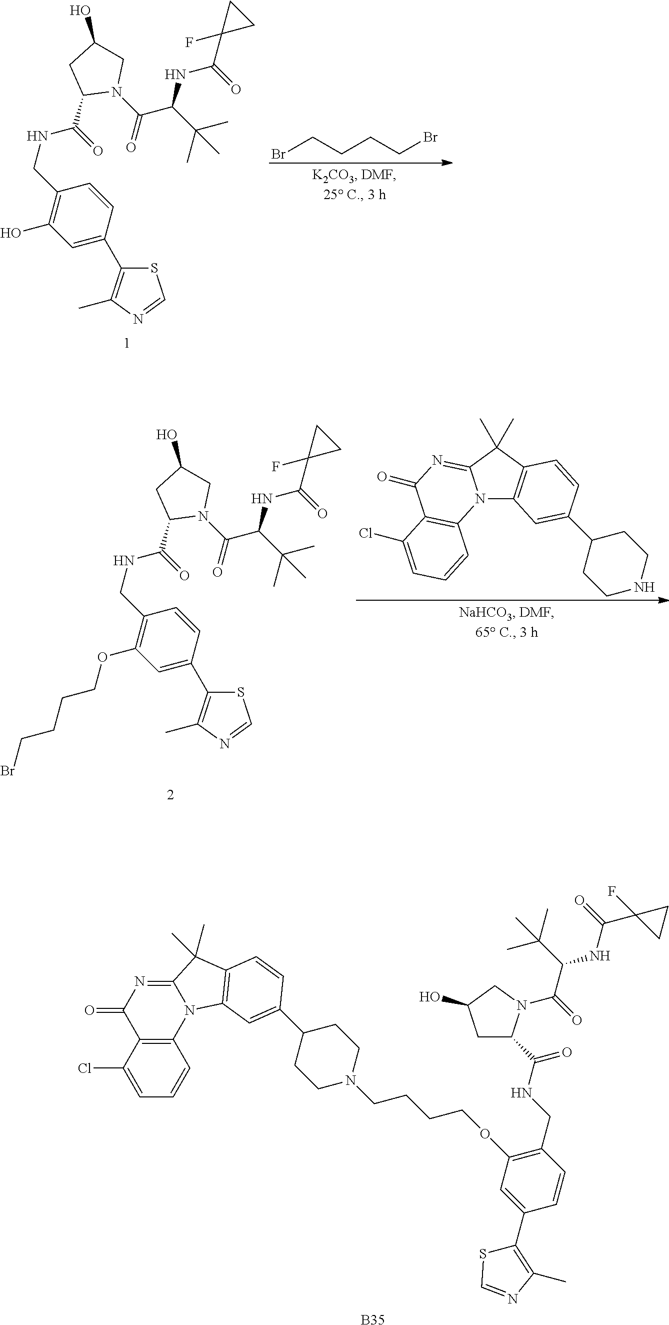 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; BrCCCCBr ; Cc1ncsc1-c1ccc(CNC(=O)[C@@H]2C[C@@H](O)CN2C(=O)[C@@H](NC(=O)C2(F)CC2)C(C)(C)C)c(OCCCCBr)c1 ; CC1(C)c2ccc(C3CCNCC3)cc2-n2c1nc(=O)c1c(Cl)cccc12 ; Cc1ncsc1-c1ccc(CNC(=O)[C@@H]2C[C@@H](O)CN2C(=O)[C@@H](NC(=O)C2(F)CC2)C(C)(C)C)c(OCCCCN2CCC(c3ccc4c(c3)-n3c(nc(=O)c5c(Cl)cccc53)C4(C)C)CC2)c1 ; "B35"]

### Synthesis of (2S,4R)—N-(2-(4-bromobutoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (Intermediate 2)

To a mixture of (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (50.0 mg, 1 eq, 0.094 mmol), 1,4-dibromobutane (203 mg, 10 eq, 0.939 mmol) in DMF (2.00 mL) was added K₂CO₃ (38.9 mg, 3 eq, 0.282 mmol), The mixture was stirred at 25° C. for 16 hours. The reaction mixture was poured into water (100 mL), extracted with EA (30 mL×3). The combined organic layers were washed with saturated brine (30 mL×3), dried over anhydrous sodium sulfate, and concentrated under vacuum. The resulting residue was purified by pre-TLC (0-10% of methanol in Dichloromethane) to afford (2S,4R)—N-(2-(4-bromobutoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (30.0 mg, 0.045 mmol, 47.9%) as a yellow solid. LC purity (0.1% FA): 94.76% (UV at 254 nm)/MS: 667.2 [M+H]; Retention time: 1.64 min.

### Synthesis of (2S,4R)—N-(2-(4-(4-(4-chloro-7,7-dimethyl-5-oxo-5,7-dihydroindolo[1,2-a]quinazolin-10-yl)piperidin-1-yl)butoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (compound 35)

To a mixture of (2S,4R)—N-(2-(4-bromobutoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (30.0 mg, 1 eq, 0.045 mmol), 4-chloro-7,7-dimethyl-10-(piperidin-4-yl)indolo[1,2-a]quinazolin-5 (7H)-one (17.1 mg, 1 eq, 0.045 mmol) in DMF (2 mL) was added Sodium bicarbonate (3.77 mg, 0.018 mL, 1 eq, 0.045 mmol), the mixture was stirred at 65° C. for 3 hours, LCMS indicated completion of reaction. The mixture was filtered and the filtrate was purified by prep-HPLC (0.1% TFA) to afford (2S,4R)—N-(2-(4-(4-(4-chloro-7,7-dimethyl-5-oxo-5,7-dihydroindolo[1,2-a]quinazolin-10-yl)piperidin-1-yl)butoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (1.50 mg, 0.001 mmol, 3.16%) as a white solid. LC purity (0.03% NH₃H₂O): 91.62% (UV at 214 nm)/MS: 966.4 [M+H]; Retention time: 10.207 min. ¹H NMR (400 MHz, CD₃OD) δ 8.89-8.87 (m, 1H), 8.46-8.43 (m, 1H), 8.03-8.00 (m, 1H), 7.89-7.86 (m, 1H), 7.67-7.66 (m, 1H), 7.64-7.61 (m, 1H), 7.51-7.48 (m, 1H), 7.38-7.34 (m, 1H), 7.06-6.97 (m, 2H), 4.73 (s, 2H), 4.66-4.60 (m, 3H), 4.59-4.58 (m, 2H), 4.47 (s, 2H), 4.22-4.17 (m, 2H), 3.88-3.86 (m, 1H), 3.84-3.79 (m, 1H), 3.66-3.64 (m, 2H), 3.15 (s, 2H), 3.05-3.01 (m, 2H), 2.49 (s, 3H), 2.17-2.10 (m, 3H), 2.02-1.97 (m, 3H), 1.59 (s, 6H), 1.38-1.24 (m, 5H), 0.96 (s, 9H).

The examples in the table below were prepared according to the same method as Compound B35 using appropriate starting materials

| | Calcd. | Found | |
|---|---|---|---|
| Compound | (M + H)⁺ | (M + H)⁺ | |
| No. | m/z | m/z | ¹H NMR |
| B34 | 938.4 | 938.6 | ¹H NMR (400 MHz, CD₃OD) δ 8.89 (s, 1H), |
| | | | 8.43 (d, J = 8.4 Hz, 1H), 7.99 (s, 1H), 7.86 (t, |
| | | | J = 8.0 Hz, 1H), 7.66 (d, J = 8.0 Hz, 1H), 7.58 (d, |
| | | | J = 7.8 Hz, 1H), 7.49 (d, J = 7.6 Hz, 1H), 7.34 |
| | | | (d, J = 8.0 Hz, 1H), 7.16-7.01 (m, 2H), 4.61- |
| | | | 4.57 (m, 6H), 4.44-4.38 (m, 2H), 3.94-3.75 (m, |
| | | | 3H), 3.46-3.44 (m, 3H), 2.92-2.90 (m, 1H), 2.51 |
| | | | (s, 3H), 2.14-2.03 (m, 5H), 1.59 (s, 6H), 1.36- |
| | | | 1.27 (m, 4H), 1.06-1.04 (m, 2H), 0.97 (s, 9H). |
| B39 | 952.4 | 952.5 | ¹H NMR (400 MHz, CD3OD) δ 8.87 (s, 1H), |
| | | | 8.43 (d, J = 8.4 Hz, 1H), 7.96 (s, 1H), 7.88-7.80 |
| | | | (m, 1H), 7.65 (d, J = 8.0 Hz, 1H), 7.57 (d, J = |
| | | | 7.6 Hz, 1H), 7.47 (d, J = 7.6 Hz, 1H), 7.33 |
| | | | (d, J = 7.6 Hz, 1H), 7.04-7.01 (m, 2H), 4.74 (s, |
| | | | 1H), 4.72-4.64 (m, 1H), 4.51-4.50 (m, 1H), |
| | | | 4.45-4.44 (m, 1H), 4.17-4.15 (m, 2H), 3.96-3.89 |
| | | | (m, 1H), 3.83-3.80 (m, 1H), 3.20 (d, J = 11.2 |
| | | | Hz, 2H), 2.87-2.78 (m, 1H), 2.68-2.56 (m, 2H), |
| | | | 2.50 (s, 3H), 2.24-2.16 (m, 2H), 2.18-2.10 (m, |
| | | | 2H), 2.04-1.94 (m, 4H), 1.59 (s, 6H), 1.36-1.28 |
| | | | (m, 5H), 1.03 (s, 9H). |
| B80 | 1006.4 | 1006.4 | ¹H NMR (400 MHz, DMSO) δ 11.05 (s, 1H), |
| | | | 8.48 (s, 1H), 8.15 (s, 1H), 7.97-7.92 (m, 1H), |
| | | | 7.86-7.81 (m, 1H), 7.67-7.59 (m, 1H), 7.58-7.53 |
| | | | (m, 1H), 7.30-7.25 (m, 1H), 7.21-7.15 (m, 1H), |
| | | | 7.00-6.95 (m, 1H), 6.67-6.62 (m, 1H), 5.35-5.29 |
| | | | (m, 2H), 5.07-4.98 (m, 1H), 3.19-3.12 (m, 2H), |
| | | | 3.08-3.06 (m, 2H), 2.69-2.65 (m, 1H), 2.35-2.3 |
| | | | (m, 2H), 2.04-1.95 (m, 6H), 1.85-1.67 (m, 5H), |
| | | | 1.67-1.52 (m, 2H), 1.50-1.23 (m, 6H), 0.88-0.82 |
| | | | (m, 3H). |

### Compound B42. (2S,4R)-1-(2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide

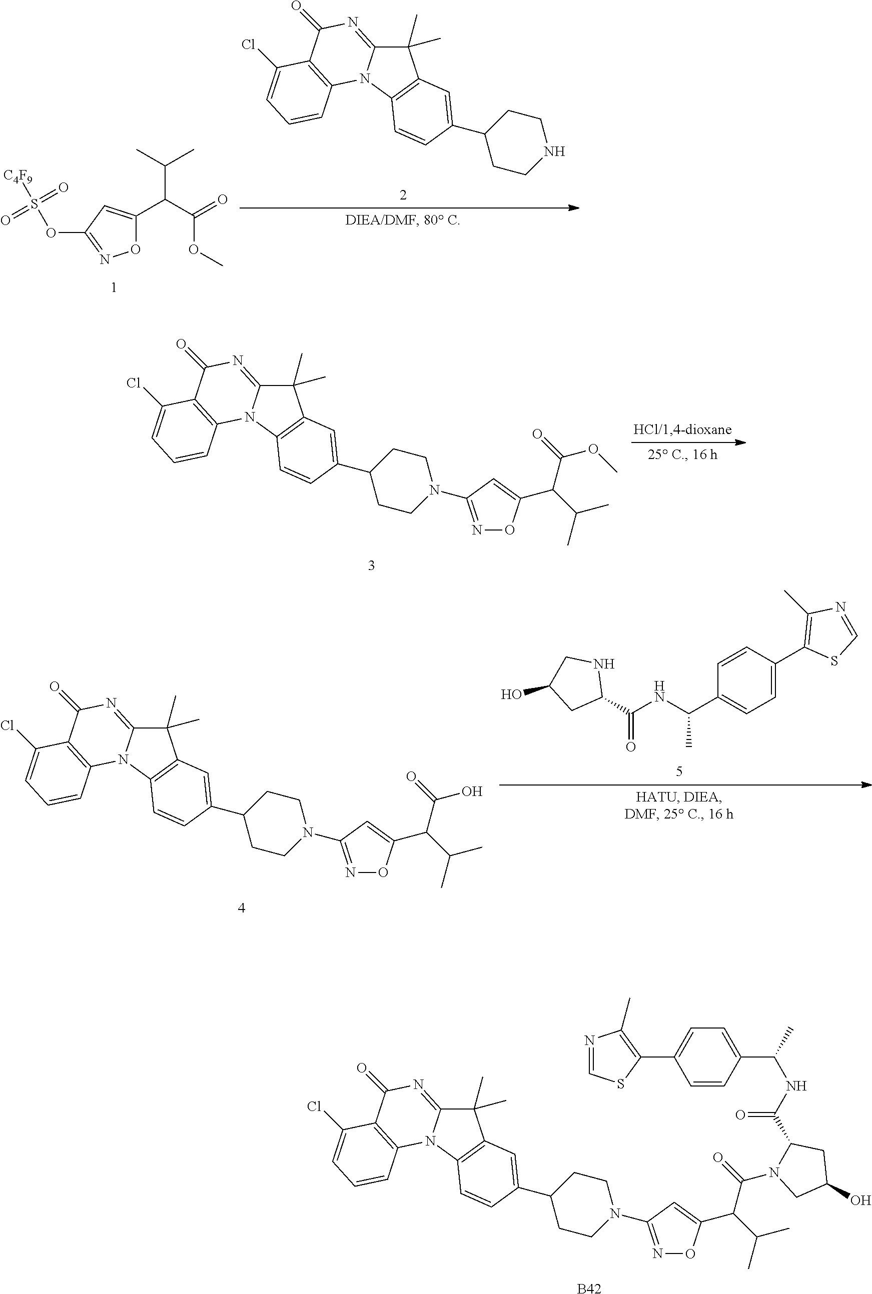

### Synthesis of methyl methyl 2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoate (Intermediate 3)

To a solution of methyl 3-methyl-2-(3-(((4,4,4,4,4,4,4,4,4-nonafluoro-4112-buta-1,3-diyn-1-yl) sulfonyl)oxy)isoxazol-5-yl)butanoate (80.0 mg, 1 eq, 166 μmol) in DMF (6 mL) was added 4-chloro-7,7-dimethyl-9-(piperidin-4-yl)indolo[1,2-a]quinazolin-5 (7H)-one (126 mg, 2 eq, 332 μmol) and diisopropylethylamine (64.3 mg, 3.0 eq, 499 μmol). The mixture was stirred at 80° C. for 4 hours. LCMS indicated completion of reaction. The reaction was poured into water (50 mL) as added, extracted with DCM (50 mL×3). The combined organic layer was washed with brine (30 mL), dried over anhydrous Anhydrous sodium sulfate, filtered and concentrated under vacuo. The residue was purified by silica gel column chromatography (100-200 mesh silica gel, 30% of EA in PE) to afford methyl 2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoate (15.0 mg, 26.7 μmol, 16.1%) as yellow oil. LC purity (0.1% FA): 58.83% (UV at 254 nm)/MS: 561.2 [M+H]; Retention time: 1.75 min.

### Synthesis of 2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoic acid (Intermediate 4)

To a solution of methyl 2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoate (20.00 mg, 1 eq, 35.65 μmol) in 1,4-dioxane (0.10 mL) was added HCl (88 mg, 0.4 mL, 6.00 molar, 67 eq, 2.4 mmol). The mixture was stirred at 90° C. for 2 hours. The solution was stirred at room temperature for 16 hours. LCMS indicated completion of reaction. The reaction mixture was concentrated under vacuum at 100C to give 2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoic acid (20.0 mg, 26 μmol, 72%, 70% purity) as a white solid. LC purity (0.1% FA): 81.69% (UV at 254 nm)/MS: 547.2 [M+1]; Retention time: 1.58 min.

### Synthesis of (2S,4R)-1-(2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide

To a solution of 2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoic acid (10.0 mg, 1 eq, 18.3 μmol) in DMF (2 mL) was added DIEA (7.09 mg, 9.55 μL, 3 eq, 54.8 μmol), 2-(3H-[1,2,3]triazolo[4,5-b]pyridin-3-yl)-1,1,3,3-tetramethylisouronium hexafluorophosphate (V) (8.34 mg, 1.2 eq, 21.9 μmol) and (2S,4R)-4-hydroxy-N—((S)-1-(4-(4-methylthiazol-5-yl)phenyl)ethyl)pyrrolidine-2-carboxamide (7.27 mg, 1.2 eq, 21.9 μmol). The mixture was stirred at 25° C. for 16 hours. LCMS indicated completion of reaction. The mixture was poured into water (20 mL), extracted with ethyl acetate (20 mL×3). The combined organic layers were washed with brine (50.0 mL), dried over sodium sulfate, filtered and concentrated under vacuum. The mixture was concentrated under vacuum and the residue was purified by prep-HPLC, eluted with CH₃CN in H₂O (0.1% FA) to afford (2S,4R)-1-(2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (4.20 mg, 4.82 μmol, 26.4%, 98.71% purity) as a white solid. LC purity (0.1% FA): 100.00% (UV at 254 nm)/MS: 860.4 [M+1]; Retention time: 1.59 min. ¹H NMR (400 MHz, CD₃OD) δ 8.92-8.79 (m, 1H), 8.96-8.72 (m, 1H), 8.42-8.49 (dd, J=8.4, 4.6 Hz, 1H), 8.17-7.97 (m, 1H), 8.13-8.02 (m, 1H), 7.88-7.77 (m, 1H), 7.67-7.61 (m, 1H), 7.56-7.67 (d, J=8.9 Hz, 1H), 7.48-7.30 (m, 5H), 6.22-6.07 (m, 1H), 5.08-4.99 (m, 1H), 4.62-4.52 (m, 3H), 4.45 (s, 1H), 3.85-3.89 (d, J=4.8 Hz, 2H), 3.75-3.61 (m, 2H), 3.07-2.95 (m, 2H), 2.87-2.92 (d, J=12.0 Hz, 1H), 2.49-2.42 (m, 3H), 2.21-2.29 (d, J=10.8 Hz, 1H), 2.02-1.79 (m, 5H), 1.63-1.57 (m, 6H), 1.56-1.45 (m, 3H), 1.14-1.02 (m, 3H), 0.98-0.90 (m, 3H).

Compound 56. (2S,4R)—N—((S)-6-(4-(4-chloro-7,7-dimethyl-5-oxo-5,7-dihydroindolo[1,2-a]quinazolin-9-yl)-1H-pyrazol-1-yl)-1-(4-(4-methylthiazol-5-yl)phenyl) hexyl)-1-((S)-2-(1-fluorocyclopropane-1-carboxamido)-3,3-dimethylbutanoyl)-4-hydroxypyrrolidine-2-carboxamide

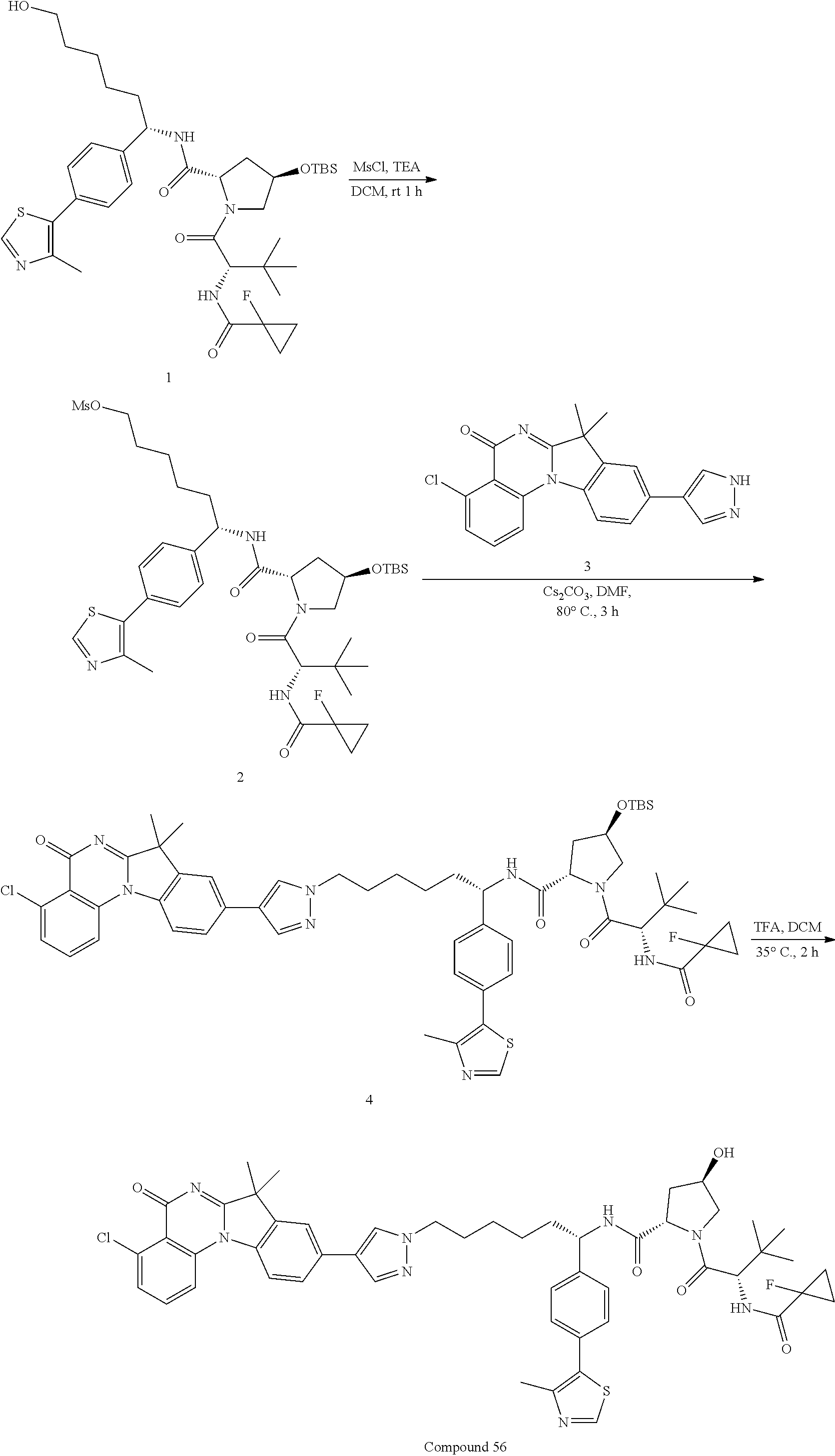

### Synthesis of(S)-6-((2S,4R)-4-((tert-butyldimethylsilyl)oxy)-1-((S)-2-(1-fluorocyclopropane-1-carboxamido)-3,3-dimethylbutanoyl)pyrrolidine-2-carboxamido)-6-(4-(4-methylthiazol-5-yl)phenyl) hexyl methanesulfonate (Intermediate 2)

To a solution of (2S,4R)-4-((tert-butyldimethylsilyl)oxy)-1-((S)-2-(1-fluorocyclopropane-1-carboxamido)-3,3-dimethylbutanoyl)-N—((S)-6-hydroxy-1-(4-(4-methylthiazol-5-yl)phenyl) hexyl)pyrrolidine-2-carboxamide (50.0 mg, 1 eq, 69.7 μmol) in DCM (1 mL) was added Methanesulfonyl chloride (9.58 mg, 6.48 μL, 1.2 eq, 83.7 μmol) and TEA (11.3 mg, 15.6 μL, 1.6 eq, 112 μmol). And the reaction was stirred at 20° C. for 5 hours. LCMS showed the reaction was completed. The reaction was poured into water (20 mL) and extracted with ethyl acetate (20 mL×2). The combined organic layers were washed with brine, dried over sodium sulfate, filtered and concentrated under vacuum to give(S)-6-((2S,4R)-4-((tert-butyldimethylsilyl)oxy)-1-((S)-2-(1-fluorocyclopropane-1-carboxamido)-3,3-dimethylbutanoyl)pyrrolidine-2-carboxamido)-6-(4-(4-methylthiazol-5-yl)phenyl) hexyl methanesulfonate (60.0 mg, 64 μmol, 92%) as a white solid. LC purity (0.1% FA): 85% (UV at 254 nm)/MS: 795.4 [M+H]; Retention time: 1.76 min.

### Synthesis of (2S,4R)-4-((tert-butyldimethylsilyl)oxy)-N—((S)-6-(4-(4-chloro-7,7-dimethyl-5-oxo-5,7-dihydroindolo[1,2-a]quinazolin-9-yl)-1H-pyrazol-1-yl)-1-(4-(4-methylthiazol-5-yl)phenyl) hexyl)-1-((S)-2-(1-fluorocyclopropane-1-carboxamido)-3,3-dimethylbutanoyl)pyrrolidine-2-carboxamide (Intermediate 4)

To a solution of(S)-6-((2S,4R)-4-((tert-butyldimethylsilyl)oxy)-1-((S)-2-(1-fluorocyclopropane-1-carboxamido)-3,3-dimethylbutanoyl)pyrrolidine-2-carboxamido)-6-(4-(4-methylthiazol-5-yl)phenyl) hexyl methanesulfonate (60.0 mg, 1 eq, 75.5 μmol) in DMF (1 mL) was added 4-chloro-7,7-dimethyl-9-(1H-pyrazol-4-yl)indolo[1,2-a]quinazolin-5 (7H)-one (30.1 mg, 1.1 eq, 83.0 μmol) and Cesium carbonate (49.2 mg, 12.1 μL, 2 eq, 151 μmol). The reaction was stirred at 80° C. for 3 hours. LCMS showed the reaction was completed. The reaction was poured into water (20 mL) and extracted with ethyl acetate (20 mL×2). The combined organic layers were washed with brine, dried over sodium sulfate, filtered and concentrated under vacuum to give (2S,4R)-4-((tert-butyldimethylsilyl)oxy)-N—((S)-6-(4-(4-chloro-7,7-dimethyl-5-oxo-5,7-dihydroindolo[1,2-a]quinazolin-9-yl)-1H-pyrazol-1-yl)-1-(4-(4-methylthiazol-5-yl)phenyl) hexyl)-1-((S)-2-(1-fluorocyclopropane-1-carboxamido)-3,3-dimethylbutanoyl)pyrrolidine-2-carboxamide (110 mg, 55 μmol, 73%, 53% Purity) as yellow oil. LC purity (0.1% FA): 53% (UV at 254 nm)/MS: 1061.5 [M+H]; Retention time: 1.86 min.

### Synthesis of (2S,4R)—N—((S)-6-(4-(4-chloro-7,7-dimethyl-5-oxo-5,7-dihydroindolo[1,2-a]quinazolin-9-yl)-1H-pyrazol-1-yl)-1-(4-(4-methylthiazol-5-yl)phenyl) hexyl)-1-((S)-2-(1-fluorocyclopropane-1-carboxamido)-3,3-dimethylbutanoyl)-4-hydroxypyrrolidine-2-carboxamide (compound 56)

A mixture of (2S,4R)-4-((tert-butyldimethylsilyl)oxy)-N—((S)-6-(4-(4-chloro-7,7-dimethyl-5-oxo-5,7-dihydroindolo[1,2-a]quinazolin-9-yl)-1H-pyrazol-1-yl)-1-(4-(4-methylthiazol-5-yl)phenyl) hexyl)-1-((S)-2-(1-fluorocyclopropane-1-carboxamido)-3,3-dimethylbutanoyl)pyrrolidine-2-carboxamide (50.0 mg, 1 eq, 47.1 μmol) in TFA (0.50 mL) and DCM (1.50 mL) was stirred at 20° C. for 3 hours. LCMS showed the reaction was completed. The reaction was purified by prep-HPLC, eluted with MeCN in H₂O (0.1% FA) to afford (2S,4R)—N—((S)-6-(4-(4-chloro-7,7-dimethyl-5-oxo-5,7-dihydroindolo[1,2-a]quinazolin-9-yl)-1H-pyrazol-1-yl)-1-(4-(4-methylthiazol-5-yl)phenyl) hexyl)-1-((S)-2-(1-fluorocyclopropane-1-carboxamido)-3,3-dimethylbutanoyl)-4-hydroxypyrrolidine-2-carboxamide (1.83 mg, 1.91 μmol, 4.06%) as a white solid. LC purity (0.1% FA): 99.00% (UV at 254 nm)/MS: 947.4 [M+H]; Retention time: 1.700 min. ¹H NMR (400 MHz, DMSO-d₆) δ 8.97 (s, 1H), 8.46-8.49 (d, J=8.8 Hz, 1H), 8.42 (d, J=8.8 Hz, 1H), 8.29 (s, 1H), 8.12 (d, J=8.8 Hz, 1H), 7.98 (s, 1H), 7.94 (d, J=2.0 Hz, 1H), 7.83 (t, J=8.0 Hz, 1H), 7.68-7.62 (m, 2H), 7.43-7.25 (m, 5H), 5.13 (d, J=3.6 Hz, 1H), 4.84-4.78 (m, 1H), 4.58 (d, J=8.8 Hz, 1H), 4.50 (t, J=8.0 Hz, 1H), 4.29 (s, 1H), 4.14 (t, J=6.8 Hz, 2H), 3.63-3.55 (m, 2H), 2.44 (s, 3H), 2.07-2.00 (m, 1H), 1.86-1.64 (m, 5H), 1.55 (s, 6H), 1.41-1.12 (m, 8H), 0.96 (s, 9H).

### Compound B43. (2S,4R)-1-((S)-2-(3-(4-(4-chloro-7,7-dimethyl-5-oxo-5,7-dihydroindolo[1,2-a]quinazolin-10-yl)piperidin-1-yl) propanamido)-3,3-dimethylbutanoyl)-4-hydroxy-N—((S)-1-(4-(4-methylthiazol-5-yl)phenyl)ethyl)pyrrolidine-2-carboxamide

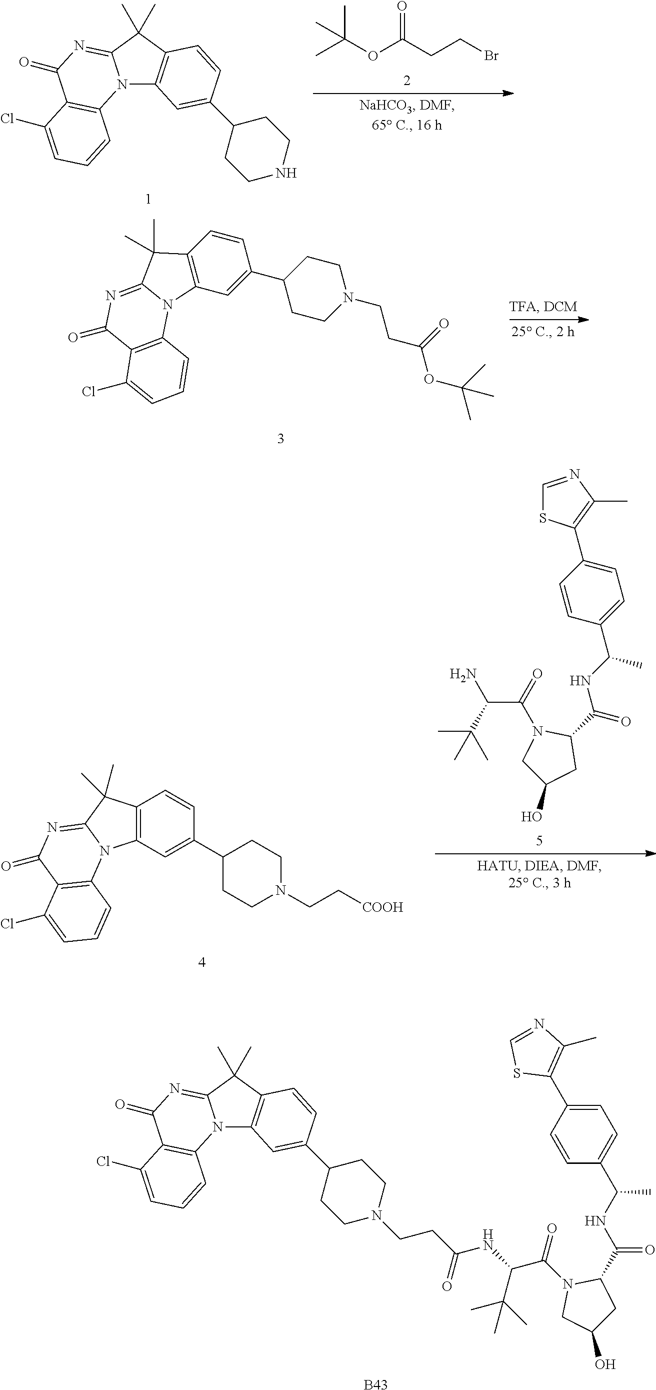

### Synthesis of tert-butyl 3-(4-(4-chloro-7,7-dimethyl-5-oxo-5,7-dihydroindolo[1,2-a]quinazolin-10-yl)piperidin-1-yl) propanoate (Intermediate 3)

To a solution of 4-chloro-7,7-dimethyl-10-(piperidin-4-yl)indolo[1,2-a]quinazolin-5 (7H)-one (50.0 mg, 1.0 eq, 132 μmol) in DMF (1 mL) was added Sodium bicarbonate (111 mg, 51.2 μL, 10 eq, 1.32 mmol) and tert-butyl 3-bromopropanoate (41.3 mg, 1.5 eq, 197 μmol). The mixture was stirred at 65° C. for 3 hours. LCMS showed the reaction was completed. The reaction was poured into H₂O (20 mL) and extracted with EA (20 mL×3). The combined organic layers were washed with saturated brine (20 mL×3), dried over sodium sulfate and concentrated under vacuum. The resulting residue was purified by silica gel column chromatography (100-200 mesh silica gel, 0-100% of ethyl acetate in petroleum ether) to get tert-butyl 3-(4-(4-chloro-7,7-dimethyl-5-oxo-5,7-dihydroindolo[1,2-a]quinazolin-10-yl)piperidin-1-yl) propanoate (40.0 mg, 72.0 mmol, 59.81%, 92% Purity) as a yellow solid. LC purity (0.03% TFA): 47.41% (UV at 254 nm)/MS: 509.3 [M+H]. Retention time: 0.859 min.

### Synthesis of 3-(4-(4-chloro-7,7-dimethyl-5-oxo-5,7-dihydroindolo[1,2-a]quinazolin-10-yl)piperidin-1-yl) propanoic acid (Intermediate 4)

a solution of tert-butyl 3-(4-(4-chloro-7,7-dimethyl-5-oxo-5,7-dihydroindolo[1,2-a]quinazolin-10-yl)piperidin-1-yl) propanoate (46.0 mg, 1 eq, 90.5 μmol) in DCM (1 mL) and TFA (1 mL) was stirred at 25° C. for 2 hours. LCMS showed the reaction was completed. The reaction was concentrated to get 3-(4-(4-chloro-7,7-dimethyl-5-oxo-5,7-dihydroindolo[1,2-a]quinazolin-10-yl)piperidin-1-yl) propanoic acid (40.0 mg, 71 μmol, 78%, 80% Purity) as a yellow oil. LC purity (0.03% TFA): 88.46% (UV at 254 nm)/MS: 452.1 [M+H]; Retention time: 0.372 min.

### Synthesis of (2S,4R)-1-((S)-2-(3-(4-(4-chloro-7,7-dimethyl-5-oxo-5,7-dihydroindolo[1,2-a]quinazolin-10-yl)piperidin-1-yl) propanamido)-3,3-dimethylbutanoyl)-4-hydroxy-N—((S)-1-(4-(4-methylthiazol-5-yl)phenyl)ethyl)pyrrolidine-2-carboxamide (compound B43)

To a solution of 3-(4-(4-chloro-7,7-dimethyl-5-oxo-5,7-dihydroindolo[1,2-a]quinazolin-10-yl)piperidin-1-yl) propanoic acid (40.0 mg, 1 eq, 88.5 μmol) in DMF (1 mL) was added HATU (101 mg, 3 eq, 266 μmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (47.2 mg, 1.2 eq, 106 μmol) and DIEA (57.2 mg, 77.1 μL, 5.0 eq, 443 μmol). The mixture was stirred at 25° C. for 16 hours. LCMS showed the reaction was completed. The reaction was poured into H₂O (20 mL) and extracted with EA (20 mL×3). The combined organic layers were washed with saturated brine (20 mL×3), dried over sodium sulfate, and concentrated under vacuum. The resulting residue was purified by prep-HPLC eluted with 30% CH₃CN in H₂O (0.05% FA) to get (2S,4R)-1-((S)-2-(3-(4-(4-chloro-7,7-dimethyl-5-oxo-5,7-dihydroindolo[1,2-a]quinazolin-10-yl)piperidin-1-yl) propanamido)-3,3-dimethylbutanoyl)-4-hydroxy-N—((S)-1-(4-(4-methylthiazol-5-yl)phenyl)ethyl)pyrrolidine-2-carboxamide (1.73 mg, 1.90 μmol, 2.15%) as a white solid. LC purity (0.1% FA): 100% (UV at 254 nm)/MS: 878.6 [M+H]; Retention time: 1.340 min. ¹H NMR (400 MHz, MeOH-d₄) δ 8.85-8.89 (d, J=5.8 Hz, 1H), 8.47-8.51 (d, J=8.9 Hz, 1H), 8.02-8.17 (d, J=20.1 Hz, 1H), 7.85-7.97 (dd, J=18.3, 9.9 Hz, 1H), 7.68-7.72 (d, J=7.9 Hz, 1H), 7.55 (d, J=7.7 Hz, 1H), 7.41-7.30 (m, 5H), 4.74 (d, J=6.8 Hz, 1H), 4.69 (s, 1H), 4.63-4.53 (m, 4H), 4.43 (s, 1H), 3.88 (d, J=10.8 Hz, 1H), 3.77 (d, J=6.8 Hz, 1H), 3.18 (d, J=11.6 Hz, 2H), 2.76 (d, J=6.4 Hz, 2H), 2.53-2.45 (m, 2H), 2.44 (s, 3H), 2.16 (s, 2H), 1.96 (d, J=8.4 Hz, 4H), 1.58 (d, J=8.9 Hz, 6H), 1.28 (d, J=7.1 Hz, 3H), 1.12 (d, J=16.1 Hz, 9H).

The examples in the table below were prepared according to the same method as Compound B43 using appropriate starting materials

| | Calcd. | Found | |
|---|---|---|---|
| Compound | (M + H)⁺ | (M + H)⁺ | |
| No. | m/z | m/z | ¹H NMR |
| B45 | 892.4 | 447.3 | ¹H NMR (400 MHz, CD3OD) δ 8.87 (s, 1H), |
| | | | 8.51 (d, J = 7.2 Hz, 1H), 8.43 (d, J = 8.4 Hz, |
| | | | 1H), 7.99 (s, 1H), 7.86-7.84 (m, 1H), 7.68-7.62 |
| | | | (m, 2H), 7.45-7.63 (m, 5H), 5.01-4.99 (m, 1H), |
| | | | 4.61-4.53 (m, 2H), 4.41-4.40 (m, 1H), 3.92-3.91 |
| | | | (m, 1H), 3.78-3.76 (m, 3H), 3.48-3.30 (m, 1H), |
| | | | 3.08-3.06 (m, 5H), 3.32-3.26 (m, 5H), 2.58-2.55 |
| | | | (m, 2H), 2.45 (s, 3H), 2.25-2.23 (m, 2H), 2.16- |
| | | | 2.07 (m, 5H), 2.04-1.95 (m, 1H), 1.57 (s, 6H), |
| | | | 1.50 (d, J = 6.8 Hz, 2H), 1.08-1.03 (m, 10H). |

### Compound B48. (2S,4R)-1-((S)-2-((1r,4S)-4-((4-(4-chloro-7,7-dimethyl-5-oxo-5,7-dihydroindolo[1,2-a]quinazolin-9-yl)-1H-pyrazol-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide

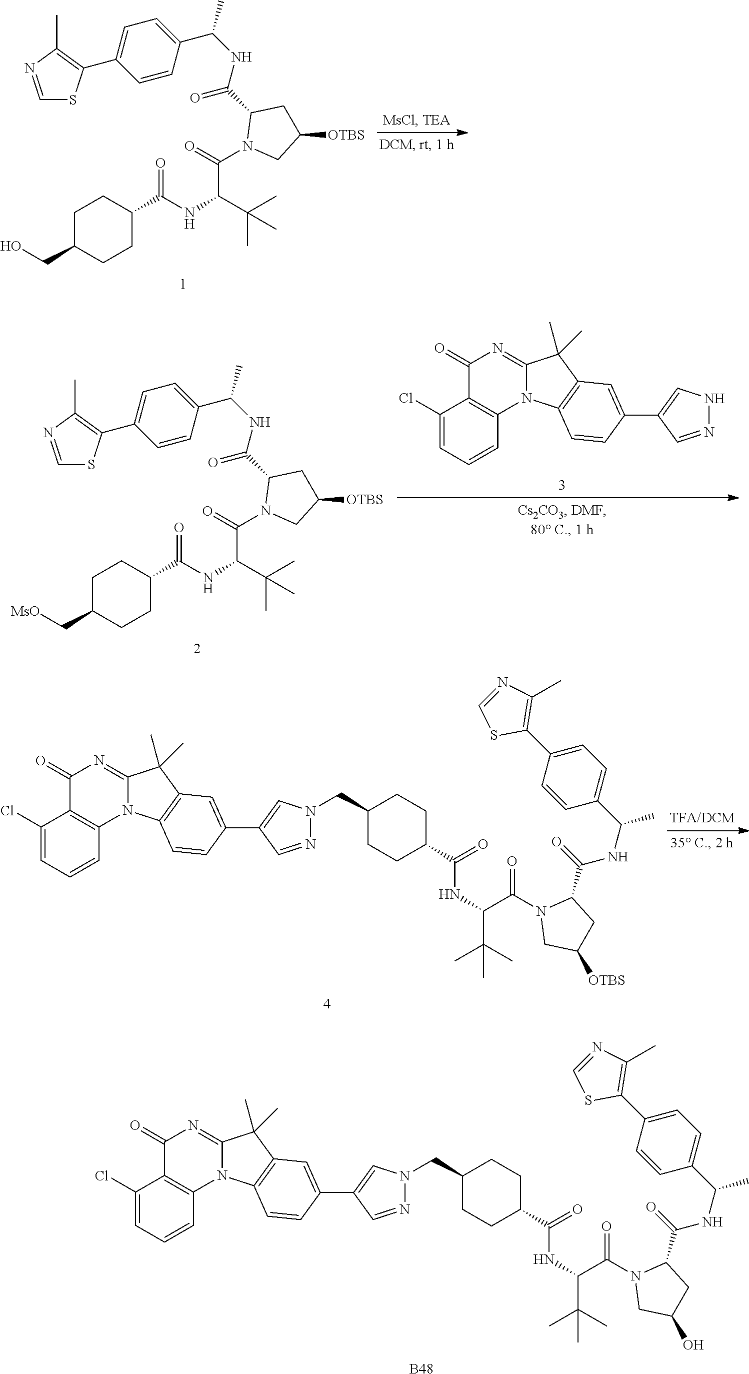

### Synthesis of ((1S,4r)-4-(((S)-1-((2S,4R)-4-((tert-butyldimethylsilyl)oxy)-2-(((S)-1-(4-(4-methylthiazol-5-yl)phenyl)ethyl) carbamoyl) pyrrolidin-1-yl)-3,3-dimethyl-1-oxobutan-2-yl) carbamoyl)cyclohexyl) methyl methanesulfonate (Intermediate 2)

To a solution of (2S,4R)-4-((tert-butyldimethylsilyl)oxy)-1-((S)-2-((1r,4S)-4-(hydroxymethyl)cyclohexane-1-carboxamido)-3,3-dimethylbutanoyl)-N—((S)-1-(4-(4-methylthiazol-5-yl)phenyl)ethyl)pyrrolidine-2-carboxamide (50.0 mg, 1 eq, 71.5 μmol) in DCM (1 mL) was added TEA (21.7 mg, 29.9 μL, 3 eq, 215 μmol) at ice water, then MsCl (9.83 mg, 6.69 μL, 1.2 eq, 85.8 μmol) was added in the mixture and stirred at 25° C. for 1 hour. LCMS indicated completion of reaction. The mixture was poured into water (20 mL), extracted with ethyl acetate (20 mL×2). The combined organic layers were washed with brine (50 mL), dried over sodium sulfate, filtered and concentrated under vacuum. The mixture was concentrated under vacuum to afford ((1S,4r)-4-(((S)-1-((2S,4R)-4-((tert-butyldimethylsilyl)oxy)-2-(((S)-1-(4-(4-methylthiazol-5-yl)phenyl)ethyl) carbamoyl) pyrrolidin-1-yl)-3,3-dimethyl-1-oxobutan-2-yl) carbamoyl)cyclohexyl) methyl methanesulfonate (46.0 mg, 30 μmol, 41%) as a yellow oil. LC purity (0.1% FA): 90% (UV at 254 nm)/MS: 777.4 [M+H]; Retention time: 1.82 min.

### Synthesis of (2S,4R)-4-((tert-butyldimethylsilyl)oxy)-1-((S)-2-((1r,4S)-4-((4-(4-chloro-7,7-dimethyl-5-oxo-5,7-dihydroindolo[1,2-a]quinazolin-9-yl)-1H-pyrazol-1-yl)methyl)cyclohexane-1-carboxamido)-3,3-dimethylbutanoyl)-N—((S)-1-(4-(4-methylthiazol-5-yl)phenyl)ethyl)pyrrolidine-2-carboxamide (Intermediate 4)

To a solution of ((1S,4r)-4-(((S)-1-((2S,4R)-4-((tert-butyldimethylsilyl)oxy)-2-(((S)-1-(4-(4-methylthiazol-5-yl)phenyl)ethyl) carbamoyl) pyrrolidin-1-yl)-3,3-dimethyl-1-oxobutan-2-yl) carbamoyl)cyclohexyl) methyl methanesulfonate (46.0 mg, 1 eq, 59.2 μmol) in DMF (1 mL) was added 4-chloro-7,7-dimethyl-9-(1H-pyrazol-4-yl)indolo[1,2-a]quinazolin-5 (7H)-one (21.5 mg, 1 eq, 59.2 μmol) and Cs₂CO₃ (57.9 mg, 3 eq, 178 μmol). The mixture was stirred at 80° C. for 1 hour under N₂. LCMS indicated completion of reaction. The mixture was poured into water (20 mL), extracted with ethyl acetate (20 mL×2). The combined organic layers were washed with brine (50 mL), dried over sodium sulfate, filtered and concentrated under vacuum. The mixture was concentrated under vacuum and the residue was purified by silica gel chromatography (100-200 mesh silica gel, 6% of MeOH in DCM) to afford (2S,4R)-4-((tert-butyldimethylsilyl)oxy)-1-((S)-2-((1r,4S)-4-((4-(4-chloro-7,7-dimethyl-5-oxo-5,7-dihydroindolo[1,2-a]quinazolin-9-yl)-1H-pyrazol-1-yl)methyl)cyclohexane-1-carboxamido)-3,3-dimethylbutanoyl)-N—((S)-1-(4-(4-methylthiazol-5-yl)phenyl)ethyl)pyrrolidine-2-carboxamide (20.0 mg, 19.2 μmol, 32.4%) as a yellow oil. LC purity (0.1% FA): 90% (UV at 254 nm)/MS: 522.4 [1/2M+H]; Retention time: 1.93 min.

### Synthesis of (2S,4R)-1-((S)-2-((1r,4S)-4-((4-(4-chloro-7,7-dimethyl-5-oxo-5,7-dihydroindolo[1,2-a]quinazolin-9-yl)-1H-pyrazol-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide (compound B48)

A mixture of (2S,4R)-4-((tert-butyldimethylsilyl)oxy)-1-((S)-2-((1r,4S)-4-((4-(4-chloro-7,7-dimethyl-5-oxo-5,7-dihydroindolo[1,2-a]quinazolin-9-yl)-1H-pyrazol-1-yl)methyl)cyclohexane-1-carboxamido)-3,3-dimethylbutanoyl)-N—((S)-1-(4-(4-methylthiazol-5-yl)phenyl)ethyl)pyrrolidine-2-carboxamide (20.0 mg, 1 eq, 19.2 μmol) in TFA (1 mL) and DCM (1 mL) was stirred at 25° C. for 1 hour. LCMS indicated completion of reaction. The mixture was concentrated under vacuum and the residue was purified by prep-HPLC (Waters 2767/Qda, Column: SunFire Sunfire C18, 19*250 mm, 10 um; Mobile Phase A: 0.1% FA/H₂O, B:ACN; flow rate: 20 ml/min; gradient: 48%˜58%; Retention Time: 8.2-8.8 min of 17 min) to afford (2S,4R)-1-((S)-2-((1r,4S)-4-((4-(4-chloro-7,7-dimethyl-5-oxo-5,7-dihydroindolo[1,2-a]quinazolin-9-yl)-1H-pyrazol-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide (1.73 mg, 1.85 μmol, 9.64%) as a white solid. LC purity (0.1% FA): 97.70% (UV at 254 nm)/MS: 465.4 [½M+H]; Retention time: 1.592 min. ¹H NMR (400 MHz, CD₃OD) δ 8.87 (s, 1H), 8.44 (d, J=8.7 Hz, 1H), 8.12 (t, J=4.3 Hz, 2H), 7.95 (s, 1H), 7.86 (t, J=8.3 Hz, 2H), 7.76-7.64 (m, 2H), 7.45-7.39 (m, 4H), 4.59-4.45 (m, 4H), 4.42 (s, 1H), 4.06 (d, J=7.1 Hz, 2H), 3.83 (s, 1H), 3.76-3.72 (m, 1H), 2.47 (s, 3H), 2.40-2.32 (m, 1H), 1.97-1.82 (m, 4H), 1.79-1.72 (m, 2H), 1.65 (s, 6H), 1.50 (d, J=7.0 Hz, 4H), 1.20-1.08 (m, 2H), 1.01 (s, 9H).

The examples in the table below were prepared according to the same method as Compound B48 using appropriate starting materials

| | Calcd. | Found | |
|---|---|---|---|
| Compound | (M + H)⁺ | (M + H)⁺ | |
| No. | m/z | m/z | ¹H NMR |
| B55 | 929.4 | 929.3 | ¹H NMR (400 MHz, CD₃OD) δ 8.88 (s, 1H), |
| | | | 8.54 (d, J = 8.6 Hz, 1H), 8.22 (d, J = 9.1 Hz, |
| | | | 2H), 8.03 (s, 1H), 7.89 (t, J = 8.3 Hz, 1H), 7.68 |
| | | | (t, J = 6.9 Hz, 2H), 7.63 (s, 2H), 7.42 (q, J = 8.5 |
| | | | Hz, 4H), 4.99 (d, J = 6.8 Hz, 1H), 4.66-4.48 (m, |
| | | | 2H), 4.42 (s, 1H), 4.06 (d, J = 7.1 Hz, 2H), 3.84 |
| | | | (d, J = 10.7 Hz, 1H), 3.77-3.66 (m, 1H), 2.47 (s, |
| | | | 3H), 2.34 (d, J = 12.3 Hz, 1H), 2.16 (d, J = 7.0 |
| | | | Hz, 1H), 2.06-1.67 (m, 7H), 1.62 (s, 6H), 1.56- |
| | | | 1.44 (m, 6H), 1.22-1.10 (m, 2H), 1.03 (s, 9H). |
| B46 | 947.4 | 947.4 | ¹H NMR (400 MHz, CD₃OD) δ 8.86 (s, 1H), |
| | | | 8.50 (d, J = 8.7 Hz, 1H), 8.28 (s, 1H), 8.20 (s, |
| | | | 1H), 8.02 (s, 1H), 7.80 (t, J = 8.3 Hz, 1H), 7.62- |
| | | | 7.60 (m, 3H), 7.49-7.33 (m, 5H), 4.90-4.88 (m, |
| | | | 1H), 4.72 (d, J = 9.5 Hz, 1H), 4.60 (t, J = 8.4 |
| | | | Hz, 1H), 4.43-4.41 (m, 1H), 4.24 (t, J = 6.8 Hz, |
| | | | 2H), 3.82 (d, J = 11.6 Hz, 1H), 3.73 (dd, J = |
| | | | 11.2, 3.50 Hz, 1H), 2.45 (s, 3H), 2.22-2.11 (m, |
| | | | 1H), 1.97-1.75 (m, 5H), 1.62 (s, 6H), 1.47-1.20 |
| | | | (m, 8H), 1.01 (s, 9H). |
| B56 | 947.4 | 947.4 | ¹H NMR (400 MHz, DMSO) δ 8.97 (s, 1H), |
| | | | 8.46 (d, J = 8.8 Hz, 1H), 8.42 (d, J = 8.8 Hz, |
| | | | 1H), 8.29 (s, 1H), 8.12 (d, J = 8.8 Hz, 1H), 7.98 |
| | | | (s, 1H), 7.94 (d, J = 2.0 Hz, 1H), 7.83 (t, J = 8.0 |
| | | | Hz, 1H), 7.68-7.62 (m, 2H), 7.43-7.25 (m, 5H), |
| | | | 5.13 (d, J = 3.6 Hz, 1H), 4.84-4.78 (m, 1H), |
| | | | 4.58 (d, J = 8.8 Hz, 1H), 4.50 (t, J = 8.0 Hz, |
| | | | 1H), 4.29 (s, 1H), 4.14 (t, J = 6.8 Hz, 2H), 3.63- |
| | | | 3.55 (m, 2H), 2.44 (s, 3H), 2.07-2.00 (m, 1H), |
| | | | 1.86-1.64 (m, 5H), 1.55 (s, 6H), 1.41-1.12 (m, |
| | | | 8H), 0.96 (s, 9H) |

### Compound B57. (2S,4R)-1-(2-(4-(2-(4-(4-chloro-7,7-dimethyl-5-oxo-5,7-dihydroindolo[1,2-a]quinazolin-9-yl)piperidin-1-yl)ethoxy)-1H-pyrazol-1-yl)-3-methylbutanoyl)-4-hydroxy-N—((S)-1-(4-(4-methylthiazol-5-yl)phenyl)ethyl)pyrrolidine-2-carboxamide

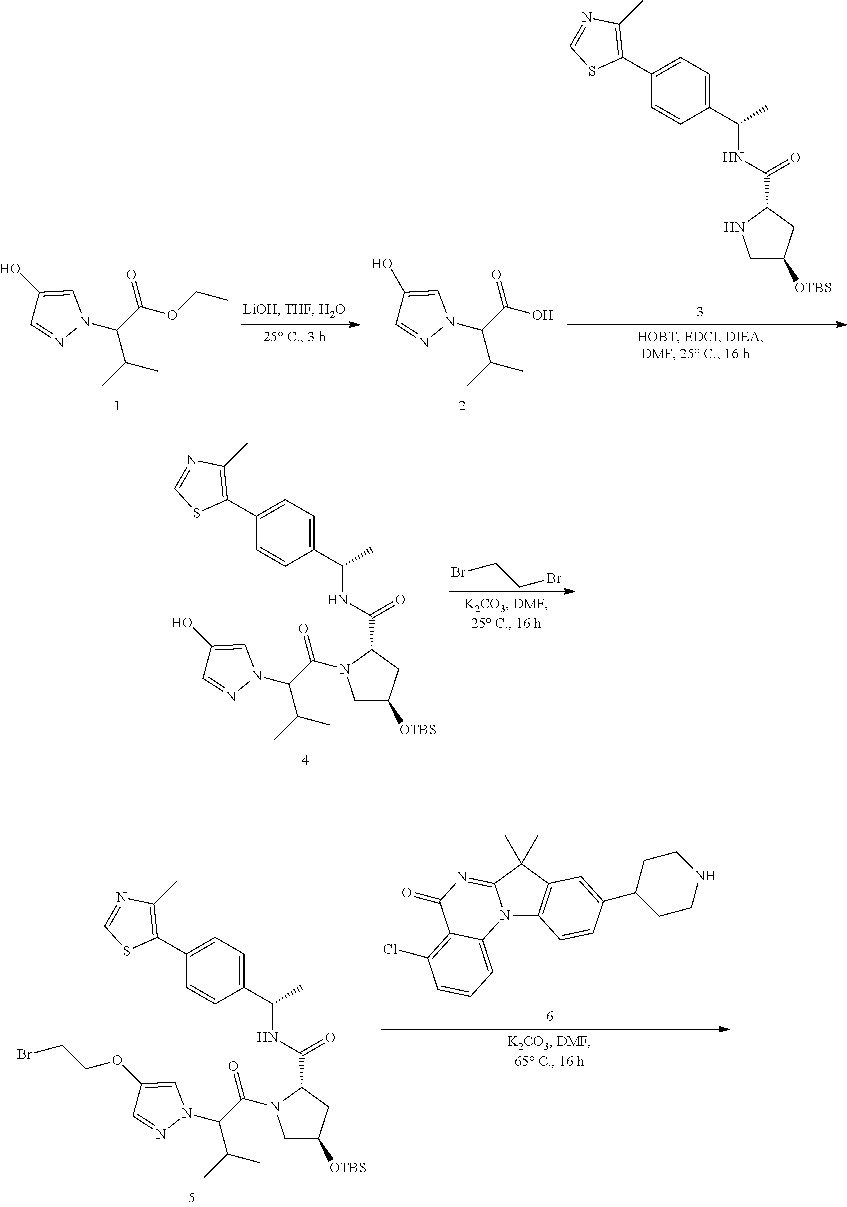

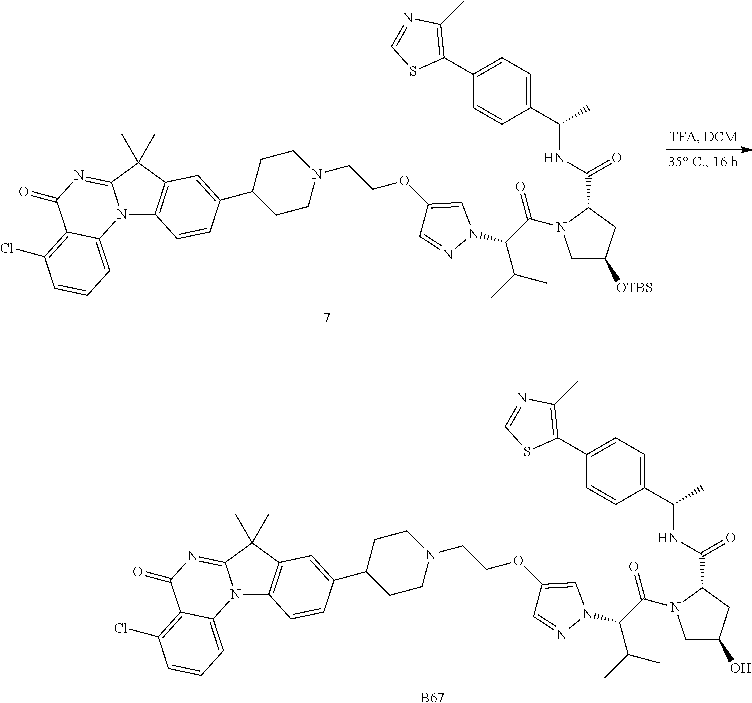

### Synthesis of 2-(4-hydroxy-1H-pyrazol-1-yl)-3-methylbutanoic acid (Intermediate 2)

To a solution of ethyl 2-(4-hydroxy-1H-pyrazol-1-yl)-3-methylbutanoate (1.05 g, 1 eq, 4.95 mmol) in Water (1 mL) and MeOH (3 mL) and THF (9 mL) was added LiOH (1.18 g, 10 eq, 49.5 mmol). The mixture was stirred at 25° C. for 1 hour. LCMS indicated completion of reaction. The reaction was filtered and concentrated to get crude 2-(4-hydroxy-1H-pyrazol-1-yl)-3-methylbutanoic acid (680 mg, 3.69 mmol, 74.6%) as a yellow solid. LC purity (0.1% NH₄HCO₃): 86.55% (UV at 254 nm)/MS: 185.0 [M+H]; Retention time: 0.521 min.

### Synthesis of (2S,4R)-4-((tert-butyldimethylsilyl)oxy)-1-(2-(3-hydroxyisoxazol-5-yl)-3-methylbutanoyl)-N—((S)-1-(4-(4-methylthiazol-5-yl)phenyl)ethyl)pyrrolidine-2-carboxamide (Intermediate 4)

To a solution of 2-(3-hydroxyisoxazol-5-yl)-3-methylbutanoic acid (680 mg, 1 eq, 3.67 mmol) in DMF (14 mL) was added EDCl (915 mg, 1.3 eq, 4.77 mmol) and N-ethyl-N-isopropylpropan-2-amine (2.37 g, 5 eq, 18.4 mmol) and 1H-benzo[d][1,2,3]triazol-1-ol hydrate (731 mg, 1.3 eq, 4.77 mmol) and (2S,4R)-4-((tert-butyldimethylsilyl)oxy)-N—((S)-1-(4-(4-methylthiazol-5-yl)phenyl)ethyl)pyrrolidine-2-carboxamide (1.64 g, 1 eq, 3.67 mmol). The mixture was stirred at 25° C. for 16 hours. LCMS indicated completion of reaction. The mixture was poured into saturated ammonium chloride solution (20 mL), extracted with EA (20 mL×2). The combined organic layer was washed with brine (50 mL), dried over sodium sulfate, filtered and concentrated in vacuum. The mixture was concentrated and the residue was purified by column chromatography on silica gel using 0-30% ethyl acetate/hexane to afford (2S,4R)-4-((tert-butyldimethylsilyl)oxy)-1-(2-(3-hydroxyisoxazol-5-yl)-3-methylbutanoyl)-N—((S)-1-(4-(4-methylthiazol-5-yl)phenyl)ethyl)pyrrolidine-2-carboxamide (800 mg, 1.31 mmol, 35.5%) as a yellow solid. LC purity (0.1% FA): 27.99% (UV at 254 nm)/MS: 612.4 [M+42]; Retention time: 1.727 min.

### Synthesis of (2S,4R)-4-((tert-butyldimethylsilyl)oxy)-1-((S)-2-(4-(2-hydroxyethoxy)-1H-pyrazol-1-yl)-3-methylbutanoyl)-N—((S)-1-(4-(4-methylthiazol-5-yl)phenyl)ethyl)pyrrolidine-2-carboxamide (Intermediate 5)

To a solution of (2S,4R)-4-hydroxy-1-(2-(4-hydroxy-1H-pyrazol-1-yl)-3-methylbutanoyl)-N—((S)-1-(4-(4-methylthiazol-5-yl)phenyl)ethyl)pyrrolidine-2-carboxamide (700 mg, 1 eq, 1.41 mmol) in DMF (10 mL) was added K₂CO₃ (583 mg, 3 eq, 4.22 mmol) and 1,2-dibromoethane (1.06 g, 4 eq, 5.63 mmol) and Sodium iodide (105 mg, 28.8 μL, 0.5 eq, 703 μmol). The mixture was stirred at 50° C. for 16 hours. LCMS indicated completion of reaction. The reaction mixture was diluted with water (20 mL), extracted with EA (20 mL×3). The combined organic layers were dried over anhydrous sodium sulfate, filtered and concentrated under vacuum. The resulting residue was purified by silica gel column chromatography (100-200 mesh silica gel, 10% of MeOH in DCM) to afford (2S,4R)-4-((tert-butyldimethylsilyl)oxy)-1-((S)-2-(4-(2-hydroxyethoxy)-1H-pyrazol-1-yl)-3-methylbutanoyl)-N—((S)-1-(4-(4-methylthiazol-5-yl)phenyl)ethyl)pyrrolidine-2-carboxamide (30.0 mg, 45.7 μmol, 3.25%) as a yellow oil. LC purity (0.1% FA): 88.25% (UV at 254 nm)/MS: 718.2 [M+H]; Retention time: 2.231 min.

### Synthesis of (2S,4R)-1-((S)-2-(4-(2-(4-(4-chloro-7,7-dimethyl-5-oxo-5,7-dihydroindolo[1,2-a]quinazolin-9-yl)piperidin-1-yl)ethoxy)-1H-pyrazol-1-yl)-3-methylbutanoyl)-4-hydroxy-N—((S)-1-(4-(4-methylthiazol-5-yl)phenyl)ethyl)pyrrolidine-2-carboxamide (Intermediate 7)

To a solution of (2S,4R)-1-((S)-2-(4-(2-bromoethoxy)-1H-pyrazol-1-yl)-3-methylbutanoyl)-4-((tert-butyldimethylsilyl)oxy)-N—((S)-1-(4-(4-methylthiazol-5-yl)phenyl)ethyl)pyrrolidine-2-carboxamide (30.0 mg, 1 eq, 41.7 μmol) in DMF (3 mL) was added Sodium bicarbonate (5.26 mg, 2.43 μL, 1.5 eq, 62.6 μmol) and 4-chloro-7,7-dimethyl-9-(piperidin-4-yl)indolo[1,2-a]quinazolin-5 (7H)-one (79.3 mg, 5 eq, 209 μmol). The mixture was stirred at 65° C. for 16 hours. LCMS indicated completion of reaction. The reaction mixture was diluted with water (20 mL), extracted with DCM (20 mL×3). The combined organic layers were dried over anhydrous sodium sulfate, filtered and concentrated under vacuum. The resulting residue was purified by silica gel column chromatography (100-200 mesh silica gel, 10% of MeOH in DCM) to afford (2S,4R)-1-((S)-2-(4-(2-(4-(4-chloro-7,7-dimethyl-5-oxo-5,7-dihydroindolo[1,2-a]quinazolin-9-yl)piperidin-1-yl)ethoxy)-1H-pyrazol-1-yl)-3-methylbutanoyl)-4-hydroxy-N—((S)-1-(4-(4-methylthiazol-5-yl)phenyl)ethyl)pyrrolidine-2-carboxamide (25.0 mg, 27.7 μmol, 66.3%) as a white oil. LC purity (0.1% FA): 19.91% (UV at 254 nm)/MS: 1018.8 [M+H]; Retention time: 1.563 min.

### Synthesis of (2S,4R)-1-(2-(4-(2-(4-(4-chloro-7,7-dimethyl-5-oxo-5,7-dihydroindolo[1,2-a]quinazolin-9-yl)piperidin-1-yl)ethoxy)-1H-pyrazol-1-yl)-3-methylbutanoyl)-4-hydroxy-N—((S)-1-(4-(4-m ethylthiazol-5-yl)phenyl)ethyl)pyrrolidine-2-carboxamide (Compound B57)

To a solution of (2S,4R)-1-(2-(4-(2-(4-(4-chloro-7,7-dimethyl-5-oxo-5,7-dihydroindolo[1,2-a]quinazolin-9-yl)piperidin-1-yl)ethoxy)-1H-pyrazol-1-yl)-3-methylbutanoyl)-4-hydroxy-N—((S)-1-(4-(4-methylthiazol-5-yl)phenyl)ethyl)pyrrolidine-2-carboxamide (25.0 mg, 1 eq, 27.7 μmol) in DCM (3 mL) was added TFA (1.48 g, 1 mL, 469 eq, 13.0 mmol). The mixture was stirred at 35° C. for 16 hours. LCMS indicated completion of reaction. The reaction mixture was concentrated under vacuum. The reaction was purified by prep-HPLC, eluted with MeCN in H₂O from 5% to 95% (0.1% FA) to afford (2S,4R)-1-(2-(4-(2-(4-(4-chloro-7,7-dimethyl-5-oxo-5,7-dihydroindolo[1,2-a]quinazolin-9-yl)piperidin-1-yl)ethoxy)-1H-pyrazol-1-yl)-3-methylbutanoyl)-4-hydroxy-N—((S)-1-(4-(4-methylthiazol-5-yl)phenyl)ethyl)pyrrolidine-2-carboxamide (1.66 mg, 1.62 μmol, 5.86%) as a white solid. LC purity (0.1% NH₄HCO₃): 100.00% (UV at 254 nm)/MS: 903.4 [M+H]; Retention time: 1.944 min. ¹H NMR (400 MHz, CD₃OD) δ 8.77-8.89 (m, 1H), 8.41-8.45 (m, 1H), 8.05-8.08 (m, 1H), 7.84-7.88 (m, 1H), 7.65-7.68 (m, 1H), 7.55-7.60 (m, 2H), 7.36-7.48 (m, 5H), 7.28-7.36 (d, J=12.4 Hz, 1H), 4.54-4.65 (m, 5H), 4.38 (s, 1H), 4.09-4.12 (m, 2H), 3.65-3.71 (t, J=14.8 Hz, 1H), 3.10-3.18 (m, 2H), 2.82-2.86 (m, 2H), 2.69 (s, 1H), 2.42-2.53 (m, 4H), 2.20-2.33 (m, 3H), 1.81-1.91 (m, 4H), 1.60-1.69 (d, J=6.4 Hz, 6H), 1.43-1.53 (m, 3H), 1.07-1.12 (m, 3H), 0.75-0.79 (m, 3H).

### Compound B59. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3-methylbutanoyl)-4-hydroxy-N-methylpyrrolidine-2-carboxamide

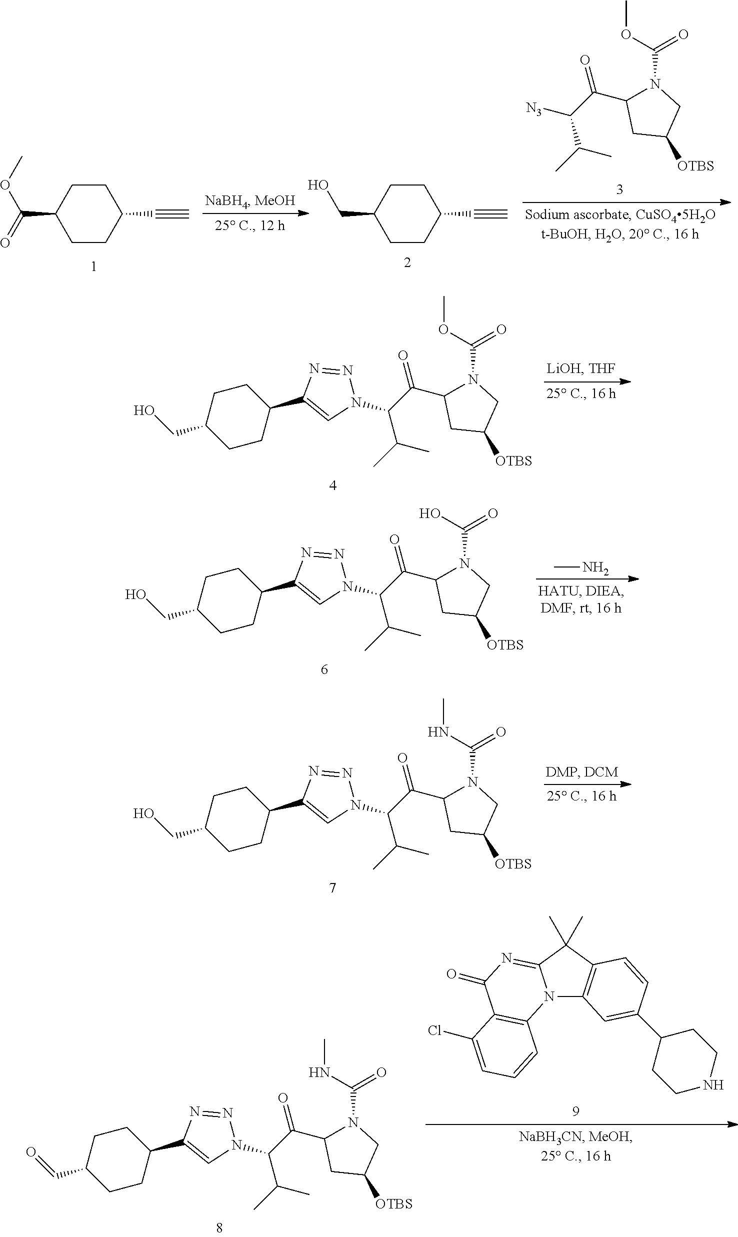

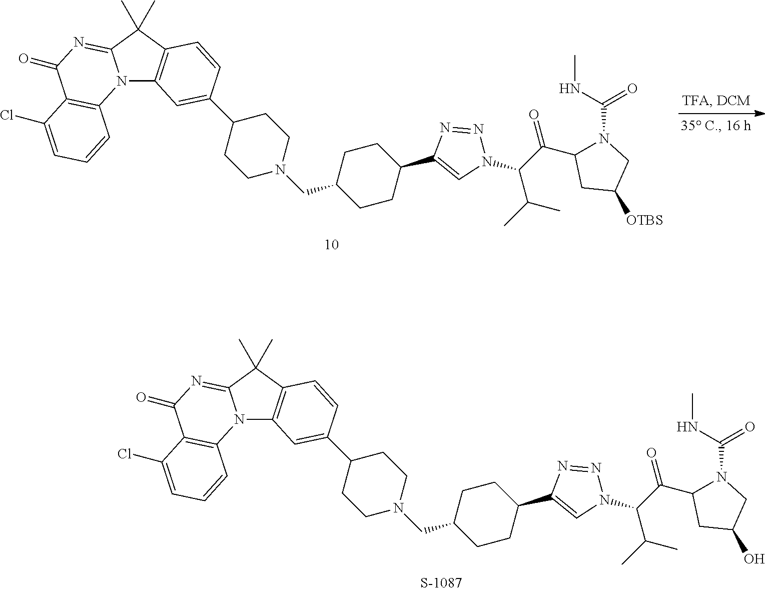

### Synthesis of ((1r,4r)-4-ethynylcyclohexyl)methanol (Intermediate 2)

To a solution of methyl (1r,4r)-4-ethynylcyclohexane-1-carboxylate (200 mg, 1 eq, 1.20 mmol) in MeOH (4 mL) was added NaBH₄ (182 mg, 4 eq, 4.81 mmol). The mixture was stirred at 25° C. for 16 hours. TLC indicated completion of reaction. The reaction mixture was diluted with water (20 mL), extracted with EA (20 mL×3). The combined organic layers were dried over anhydrous sodium sulfate, filtered and concentrated under vacuum. The resulting residue was purified by silica gel column chromatography (100-200 mesh silica gel, 20% of EA in PE) to afford ((1r,4r)-4-ethynylcyclohexyl)methanol (90.0 mg, 651 μmol, 54.1%) as a colorless oil. ¹H NMR (400 MHz, DMSO) δ 4.36 (m, 1H), 3.18-3.20 (m, 2H), 2.81-2.86 (m, 1H), 2.10-2.17 (m, 1H), 1.85-1.91 (m, 2H), 1.66-1.74 (m, 2H), 1.22-1.31 (m, 3H), 0.80-0.91 (m, 2H).

### Synthesis of (2S,4R)-4-((tert-butyldimethylsilyl)oxy)-1-((S)-2-(4-((1r,4S)-4-(hydroxymethyl)cyclohexyl)-1H-1,2,3-triazol-1-yl)-3-methylbutanoyl)pyrrolidine-2-carboxylate (Intermediate 4)

To a solution of ((1r,4r)-4-ethynylcyclohexyl)methanol (90.0 mg, 1 eq, 0.65 mmol) in Water (1 mL) and t-BuOH (1 mL) was added copper (II) sulfate (10.4 mg, 0.1 eq, 0.06 mmol) and Sodium 2-(1,2-dihydroxy-ethyl)-4-hydroxy-5-oxo-2,5-dihydro-furan-3-olate (64.5 mg, 0.5 eq, 0.3 mmol) and methyl (2S,4R)-1-((S)-2-azido-3-methylbutanoyl)-4-((tert-butyldimethylsilyl)oxy)pyrrolidine-2-carboxylate (250 mg, 1 eq, 0.65 mmol). The mixture was stirred at 25° C. for 16 hours. LCMS indicated completion of reaction. The mixture was poured into saturated ammonium chloride solution (20 mL), extracted with EA (20 mL×2). The combined organic layer was washed with brine (50 mL), dried over anhydrous sodium sulfate, filtered and concentrated under vacuum. The resulting residue was purified by silica gel column chromatography (100-200 mesh silica gel, 10% of MeOH in DCM) to afford methyl (2S,4R)-4-((tert-butyldimethylsilyl)oxy)-1-((S)-2-(4-((1r,4S)-4-(hydroxymethyl)cyclohexyl)-1H-1,2,3-triazol-1-yl)-3-methylbutanoyl)pyrrolidine-2-carboxylate (80.0 mg, 153 μmol, 0.0235%) as a white oil. LC purity (0.1% FA): 93.98% (UV at 214 nm)/MS: 523.4 [M+42]; Retention time: 1.867 min.

### Synthesis of (2S,4R)-4-((tert-butyldimethylsilyl)oxy)-1-((S)-2-(4-((1r,4S)-4-(hydroxymethyl)cyclohexyl)-1H-1,2,3-triazol-1-yl)-3-methylbutanoyl)pyrrolidine-2-carboxylic acid (Intermediate 6)

To a solution of methyl (2S,4R)-4-((tert-butyldimethylsilyl)oxy)-1-((S)-2-(4-((1r,4S)-4-(hydroxymethyl)cyclohexyl)-1H-1,2,3-triazol-1-yl)-3-methylbutanoyl)pyrrolidine-2-carboxylate (70.0 mg, 1 eq, 134 μmol) in Water (0.3 mL) and MeOH (1 mL) and THF (3 mL) was added LiOH (32.1 mg, 10 eq, 1.34 mmol). The mixture was stirred at 25° C. for 2 hours. LCMS indicated completion of reaction. The reaction mixture was diluted with water (20 mL), extracted with EA (20 mL×3). The combined organic layers were dried over anhydrous sodium sulfate, filtered and concentrated under vacuum to afford (2S,4R)-4-((tert-butyldimethylsilyl)oxy)-1-((S)-2-(4-((1r,4S)-4-(hydroxymethyl)cyclohexyl)-1H-1,2,3-triazol-1-yl)-3-methylbutanoyl)pyrrolidine-2-carboxylic acid (60.0 mg, 118 μmol, 88.1%) as a yellow solid. LC purity (0.1% FA): 48.57% (UV at 214 nm)/MS: 509.4 [M+H]; Retention time: 1.556 min.

### Synthesis of (2S,4R)-4-((tert-butyldimethylsilyl)oxy)-1-((S)-2-(4-((1r,4S)-4-(hydroxymethyl)cyclohexyl)-1H-1,2,3-triazol-1-yl)-3-methylbutanoyl)-N-methylpyrrolidine-2-carboxamide (Intermediate 7)

To a solution of (2S,4R)-4-((tert-butyldimethylsilyl)oxy)-1-((S)-2-(4-((1r,4S)-4-(hydroxymethyl)cyclohexyl)-1H-1,2,3-triazol-1-yl)-3-methylbutanoyl)pyrrolidine-2-carboxylic acid (70.0 mg, 1 eq, 0.13 mmol) in DMF (3 mL) was added HATU (78.5 mg, 1.5 eq, 0.2 mmol) and Methylamine hydrochloride (13.9 mg, 1.5 eq, 0.2 mmol) and TEA (69.6 mg, 5 eq, 0.68 mmol). The mixture was stirred at 25° C. for 16 hours. LCMS indicated completion of reaction. The reaction mixture was diluted with water (20 mL), extracted with DCM (20 mL×3). The combined organic layers were dried over anhydrous sodium sulfate, filtered and concentrated under vacuum. The resulting residue was purified by silica gel column chromatography (100-200 mesh silica gel, 10% of MeOH in DCM) to afford (2S,4R)-4-((tert-butyldimethylsilyl)oxy)-1-((S)-2-(4-((1r,4S)-4-(hydroxymethyl)cyclohexyl)-1H-1,2,3-triazol-1-yl)-3-methylbutanoyl)-N-methylpyrrolidine-2-carboxamide (35.0 mg, 67.1 μmol, 48.7%) as a yellow oil. LC purity (0.1% FA): 28.37% (UV at 214 nm)/MS: 522.4 [M+H]; Retention time: 1.534 min.

### Synthesis of (2S,4R)-4-((tert-butyldimethylsilyl)oxy)-1-((S)-2-(4-((1r,4S)-4-formylcyclohexyl)-1H-1,2,3-triazol-1-yl)-3-methylbutanoyl)-N-methylpyrrolidine-2-carboxamide (Intermediate 8)

To a solution of (2S,4R)-4-((tert-butyldimethylsilyl)oxy)-1-((S)-2-(4-((1r,4S)-4-(hydroxymethyl)cyclohexyl)-1H-1,2,3-triazol-1-yl)-3-methylbutanoyl)-N-methylpyrrolidine-2-carboxamide (35.0 mg, 1 eq, 67.1 μmol) in DCM (3 mL) was added dess-martinperiodinane (56.9 mg, 2 eq, 134 μmol) in 0° C. The mixture was stirred at 25° C. for 16 hours. LCMS indicated completion of reaction. The reaction mixture was diluted with water (20 mL), extracted with EA (20 mL×3). The combined organic layers were dried over anhydrous sodium sulfate, filtered and concentrated. The resulting residue was purified by silica gel column chromatography (100-200 mesh silica gel, 10% of MeOH in DCM) to get (2S,4R)-4-((tert-butyldimethylsilyl)oxy)-1-((S)-2-(4-((1r,4S)-4-formylcyclohexyl)-1H-1,2,3-triazol-1-yl)-3-methylbutanoyl)-N-methylpyrrolidine-2-carboxamide (30.0 mg, 57.7 μmol, 86.0%) as a white solid. LC purity (0.1% FA): 78.83% (UV at 214 nm)/MS: 520.4 [M+H]; Retention time: 1.655 min.

### Synthesis of (2S,4R)-4-((tert-butyldimethylsilyl)oxy)-1-((S)-2-(4-((1r,4S)-4-((4-(4-chloro-7,7-dim ethyl-5-oxo-5,7-dihydroindolo[1,2-a]quinazolin-10-yl)piperidin-1-yl)methyl)cyclohexyl)-1H-1,2,3-triazol-1-yl)-3-methylbutanoyl)-N-methylpyrrolidine-2-carboxamide (Intermediate 10)

To a solution of (2S,4R)-4-((tert-butyldimethylsilyl)oxy)-1-((S)-2-(4-((1r,4S)-4-formylcyclohexyl)-1H-1,2,3-triazol-1-yl)-3-methylbutanoyl)-N-methylpyrrolidine-2-carboxamide (30.0 mg, 1 eq, 57.7 μmol) in MeOH (3 mL) was added Sodium cyanoborohydride (5.44 mg, 5.10 μL, 1.5 eq, 86.6 μmol) and 4-chloro-7,7-dimethyl-10-(piperidin-4-yl)indolo[1,2-a]quinazolin-5 (7H)-one (21.9 mg, 1 eq, 57.7 μmol) and TEA (29.2 mg, 40.2 μL, 5 eq, 289 μmol). The mixture was stirred at 25° C. for 16 hours. LCMS indicated completion of reaction. The reaction mixture was diluted with water (20 mL), extracted with DCM (20 mL×3). The combined organic layers were dried over anhydrous sodium sulfate, filtered and concentrated. The resulting residue was purified by silica gel column chromatography (100-200 mesh silica gel, 10% of MeOH in DCM) to get (2S,4R)-4-((tert-butyldimethylsilyl)oxy)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3-methylbutanoyl)-N-methylpyrrolidine-2-carboxamide (20.0 mg, 22.6 μmol, 39.2%) as a white solid. LC purity (0.1% FA): 79.54% (UV at 254 nm)/MS: 883.4 [M+H]; Retention time: 1.430 min.

### Synthesis of (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3-methylbutanoyl)-4-hydroxy-N-methylpyrrolidine-2-carboxamide (Compound B59)

To a solution of (2S,4R)-4-((tert-butyldimethylsilyl)oxy)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3-methylbutanoyl)-N-methylpyrrolidine-2-carboxamide (25.0 mg, 1 eq, 28.3 μmol) in DCM (3 mL) was added TFA (1.48 g, 1.00 mL, 459 eq, 13.0 mmol). The mixture was stirred at 25° C. for 2 hours. LCMS indicated completion of reaction. The reaction mixture was concentrated under vacuum. The reaction was purified by prep-HPLC, eluted with MeCN in H2O from 5% to 95% (0.1% FA) to afford (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3-methylbutanoyl)-4-hydroxy-N-methylpyrrolidine-2-carboxamide (1.78 mg, 2.21 μmol, 7.79%) as a white solid. LC purity (0.1% NH₄—HCO₃): 90.01% (UV at 254 nm)/MS: 769.4 [M+H]; Retention time: 1.173 min. 1H NMR (400 MHz, CD₃OD) δ 8.42 (m, 1H), 8.02-8.04 (d, J=15.6 Hz, 1H), 7.75-7.91 (m, 2H), 7.65-7.71 (m, 2H), 7.38-7.46 (m, 1H), 5.27-5.36 (m, 1H), 4.40-4.50 (m, 1H), 4.40-4.56 (m, 1H), 3.93-3.98 (m, 1H), 3.65-3.88 (m, 3H), 3.41-3.62 (m, 1H), 3.14-3.26 (m, 4H), 2.75-2.82 (s, 3H), 2.17-2.25 (m, 6H), 1.88-2.08 (m, 6H), 1.88-1.99 (m, 1H), 1.60-1.72 (s, 6H), 1.30-1.36 (m, 3H), 1.30-1.46 (m, 3H), 0.72-0.82 (m, 3H), 0.69-0.76 (m, 1H).

### Compound B66. (2S,4R)-1-((S)-2-(4-(1-(3-(4-(4-chloro-7,7-dimethyl-5-oxo-5,7-dihydroindolo[1,2-a]quinazolin-10-yl)piperidin-1-yl)propyl)piperidin-4-yl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide

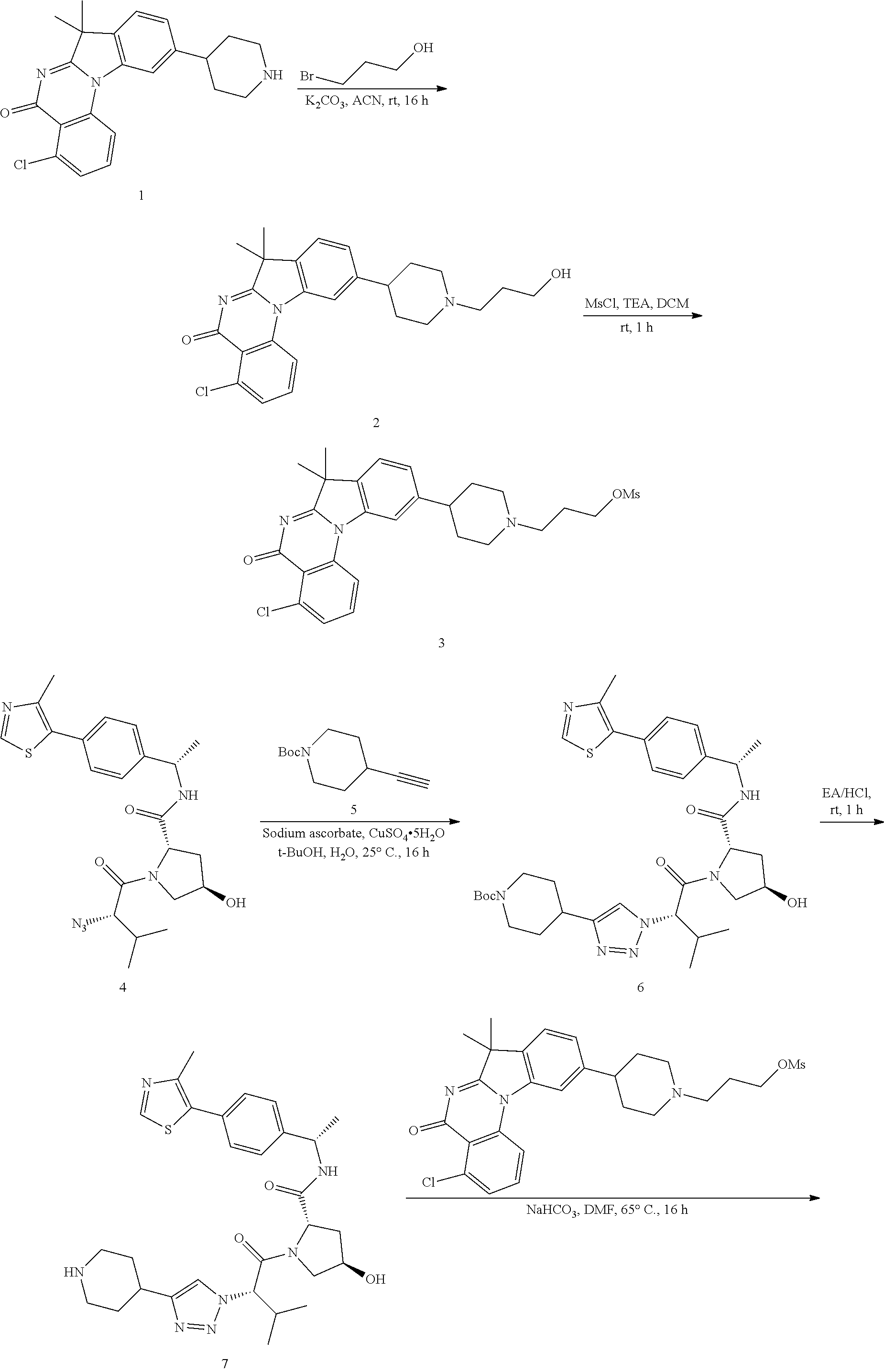

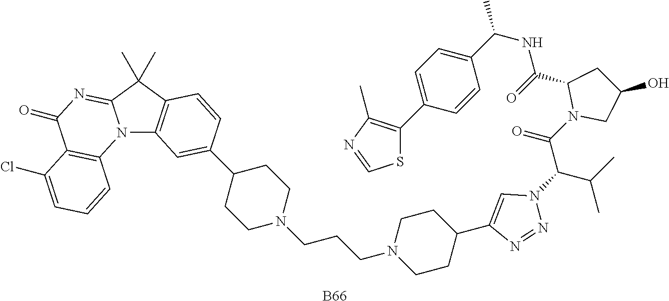

### Synthesis of 4-chloro-10-(1-(3-hydroxypropyl)piperidin-4-yl)-7,7-dimethylindolo[1,2-a]quinazolin-5 (7H)-one (Intermediate 2)

To a solution of 4-chloro-7,7-dimethyl-10-(piperidin-4-yl)indolo[1,2-a]quinazolin-5 (7H)-one (70.0 mg, 1 eq, 184 μmol) in CH₃CN (2 mL) was added K₂CO₃ (25.5 mg, 1 eq, 184 μmol), 3-bromopropan-1-ol (5.12 mg, 10 eq, 36.9 μmol). The mixture was stirred at 25° C. for 16 hours under N₂. LCMS indicated completion of reaction. The mixture was poured into water (20 mL), extracted with ethyl acetate (20 mL×2). The combined organic layers were washed with brine (50 mL), dried over sodium sulfate, filtered and concentrated under vacuum. The resulting residue was purified by silica gel chromatography (100-200 mesh silica gel, 13% of MeOH in DCM) to afford 4-chloro-10-(1-(3-hydroxypropyl)piperidin-4-yl)-7,7-dimethylindolo[1,2-a]quinazolin-5 (7H)-one (28.0 mg, 63.9 μmol, 34.7%) as a yellow oil. LC purity (0.1% FA): 90% (UV at 254 nm)/MS: 438.2 [M+H]; Retention time: 1.11 min.

### Synthesis of 3-(4-(4-chloro-7,7-dimethyl-5-oxo-5,7-dihydroindolo[1,2-a]quinazolin-10-yl)piperidin-1-yl)propyl methanesulfonate (Intermediate 3)

To a solution of 4-chloro-10-(1-(3-hydroxypropyl)piperidin-4-yl)-7,7-dimethylindolo[1,2-a]quinazolin-5 (7H)-one (28.0 mg, 1 eq, 63.9 μmol) in DCM (1 mL) was added TEA (9.70 mg, 13.4 μL, 1.5 eq, 95.9 μmol), Methanesulfonyl chloride (22.0 mg, 14.8 μL, 3 eq, 192 μmol). The mixture was stirred at 25° C. for 1 hour under N₂. LCMS indicated completion of reaction. The mixture was poured into water (20 mL), extracted with ethyl acetate (20 mL×2). The combined organic layers were washed with brine (50 mL), dried over sodium sulfate, filtered and concentrated under vacuum to afford 3-(4-(4-chloro-7,7-dimethyl-5-oxo-5,7-dihydroindolo[1,2-a]quinazolin-10-yl)piperidin-1-yl)propyl methanesulfonate (25.0 mg, 44 μmol, 68%) as a yellow oil. The resulting residue was used to the next step. LC purity (0.1% FA): 90% (UV at 254 nm)/MS: 517.2 [M+H]; Retention time: 1.114 min.

### Synthesis of tert-butyl 4-(1-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)-1H-1,2,3-triazol-4-yl)piperidine-1-carboxylate (Intermediate 6)

To a solution of (2S,4R)-1-((S)-2-azido-3-methylbutanoyl)-4-hydroxy-N—((S)-1-(4-(4-methylthiazol-5-yl)phenyl)ethyl)pyrrolidine-2-carboxamide (200 mg, 1 eq, 438 μmol) in H₂O (2 mL) and t-BuOH (2 mL) was added (2S,4R)-1-((S)-2-azido-3-methylbutanoyl)-4-hydroxy-N—((S)-1-(4-(4-methylthiazol-5-yl)phenyl)ethyl)pyrrolidine-2-carboxamide (200 mg, 1 eq, 438 μmol), tert-butyl 4-ethynylpiperidine-1-carboxylate (91.7 mg, 1 eq, 438 μmol) and Copper (II) SulfatePentahydrate (10.9 mg, 10.6 μL, 0.1 eq, 43.8 μmol). The mixture was stirred at 25° C. for 16 hours under N₂. The reaction was quenched by H₂O (50 mL), washed with EA (100 mL×3). The combined organic layers were dried over sodium sulfate, filtered and concentrated under vacuum. The resulting residue was purified by prep-TLC (MeOH/DCM=1/30) tert-butyl 4-(1-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)-1H-1,2,3-triazol-4-yl)piperidine-1-carboxylate (240 mg, 360 μmol, 82.3%) as yellow solid. LC purity (0.1% FA): 79.15% (UV at 254 nm)/MS: 666.4 [M+H]; Retention time: 1.53 min.

### Synthesis of (2S,4R)-4-hydroxy-1-((S)-3-methyl-2-(4-(piperidin-4-yl)-1H-1,2,3-triazol-1-yl)butanoyl)-N—((S)-1-(4-(4-methylthiazol-5-yl)phenyl)ethyl)pyrrolidine-2-carboxamide (Intermediate 7)

To a solution of 4-(1-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)-1H-1,2,3-triazol-4-yl)piperidine-1-carboxylate (50 mg) in EA (1 mL) was added 3M HCl/EA (5 mL). The mixture was stirred at 25° C. for 1 hour under N₂. LCMS indicated completion of reaction. The mixture was filtered and concentrated under vacuum to afford (2S,4R)-4-hydroxy-1-((S)-3-methyl-2-(4-(piperidin-4-yl)-1H-1,2,3-triazol-1-yl)butanoyl)-N—((S)-1-(4-(4-methylthiazol-5-yl)phenyl)ethyl)pyrrolidine-2-carboxamide (36.0 mg, 57 μmol, 89%) as a yellow oil. LC purity (0.1% FA): 90% (UV at 254 nm)/MS: 566.3 [M+H]; Retention time: 1.083 min.

### Synthesis of (2S,4R)-1-((S)-2-(4-(1-(3-(4-(4-chloro-7,7-dimethyl-5-oxo-5,7-dihydroindolo[1,2-a]quinazolin-10-yl)piperidin-1-yl)propyl)piperidin-4-yl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide (compound 66)

To a solution of 3-(4-(4-chloro-7,7-dimethyl-5-oxo-5,7-dihydroindolo[1,2-a]quinazolin-10-yl)piperidin-1-yl)propyl methanesulfonate (28.0 mg, 1.1 eq, 54.3 μmol) in DMF (1 mL) was added (2S,4R)-4-hydroxy-1-((S)-3-methyl-2-(4-(piperidin-4-yl)-1H-1,2,3-triazol-1-yl)butanoyl)-N—((S)-1-(4-(4-methylthiazol-5-yl)phenyl)ethyl)pyrrolidine-2-carboxamide (27.9 mg, 1 eq, 49.3 μmol), Sodium bicarbonate (12.4 mg, 5.76 μL, 3 eq, 148 μmol). The mixture was stirred at 65° C. for 16 hours under N₂. LCMS indicated completion of reaction. The mixture was poured into water (20 mL), extracted with ethyl acetate (20 mL×2). The combined organic layers were washed with brine (50 mL), dried over sodium sulfate, filtered and concentrated under vacuum. The resulting residue was purified by prep-HPLC, eluted with CH₃CN in H₂O from 10% to 95% (0.1% NH₄HCO₃) to afford (2S,4R)-1-((S)-2-(4-(1-(3-(4-(4-chloro-7,7-dimethyl-5-oxo-5,7-dihydroindolo[1,2-a]quinazolin-10-yl)piperidin-1-yl)propyl)piperidin-4-yl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide (2.69 mg, 2.42 μmol, 4.91%) as a white solid. LC purity (0.1% FA): 81.64% (UV at 254 nm)/MS: 493.4 [1/2M+H]; Retention time: 1.184 min. ¹H NMR (400 MHz, CD₃OD) δ 8.87 (s, 1H), 8.44-8.40 (d, J=7.9 Hz, 1H), 7.97 (s, 1H), 7.93 (s, 1H), 7.83-7.90 (m, 1H), 7.66 (d, J=8.0 Hz, 1H), 7.58 (d, J=7.7 Hz, 1H), 7.39-7.48 (m, 4H), 7.35-7.29 (d, J=7.3 Hz, 1H), 5.05-5.28 (m, 1H), 4.46-4.53 (m, 2H), 3.77-3.92 (m, 2H), 3.47-3.49 (m, 1H), 3.09-3.14 (m, 3H), 2.82-2.84 (m, 2H), 2.50-2.58 (m, 4H), 2.48 (s, 3H), 2.17-2.30 (m, 5H), 2.06-2.09 (m, 2H), 1.81-2.00 (m, 9H), 1.59 (s, 6H), 1.521-49 (d, J=7.0 Hz, 3H), 1.29-1.33 (s, 2H), 1.13-1.07 (d, J=6.6 Hz, 3H), 0.74 (d, J=6.6 Hz, 3H).

The examples in the table below were prepared according to the same method as Compound B66 using appropriate starting materials

| | Calcd. | Found | |
|---|---|---|---|
| Compound | (M + H)⁺ | (M + H)⁺ | |
| No. | m/z | m/z | ¹H NMR |
| B67 | 971.4 | 971.1 | ¹H NMR (400 MHz, CD₃OD) δ 8.87 (s, 1H), |
| | | | 8.43 (d, J = 8.0 Hz, 1H), 7.97 (d, J = 6.4 Hz, |
| | | | 2H), 7.90-7.83 (m, 1H), 7.67 (d, J = 8.0 Hz, |
| | | | 1H), 7.60 (d, J = 7.8 Hz, 1H), 7.48-7.39 (m, |
| | | | 4H), 7.35 (d, J = 7.7 Hz, 1H), 5.30 (d, J = 10.3 |
| | | | Hz, 1H), 5.06-5.01 (m, 1H), 4.53-4.47 (m, 2H), |
| | | | 3.91-3.82 (m, 2H), 3.42 (d, J = 10.9 Hz, 1H), |
| | | | 3.04-2.88 (m, 7H), 2.76-2.49 (m, 6H), 2.48 (s, |
| | | | 3H), 2.25-1.84 (m, 12H), 1.59 (s, 6H), 1.52 (d, |
| | | | J = 7.0 Hz, 3H), 1.13 (d, J = 6.6 Hz, 3H), 0.76 |
| | | | (d, J = 6.6 Hz, 3H). |

### Compound B93. (2S,4R)-1-((S)-2-(4-(4-(4-chloro-7,7-dimethyl-5-oxo-5,7-dihydroindolo[1,2-a]quinazolin-9-yl)-1H-pyrazol-1-yl)butanamido)-3,3-dimethylbutanoyl)-4-hydroxy-N—((S)-1-(4-(4-methylthiazol-5-yl)phenyl)ethyl)pyrrolidine-2-carboxamide

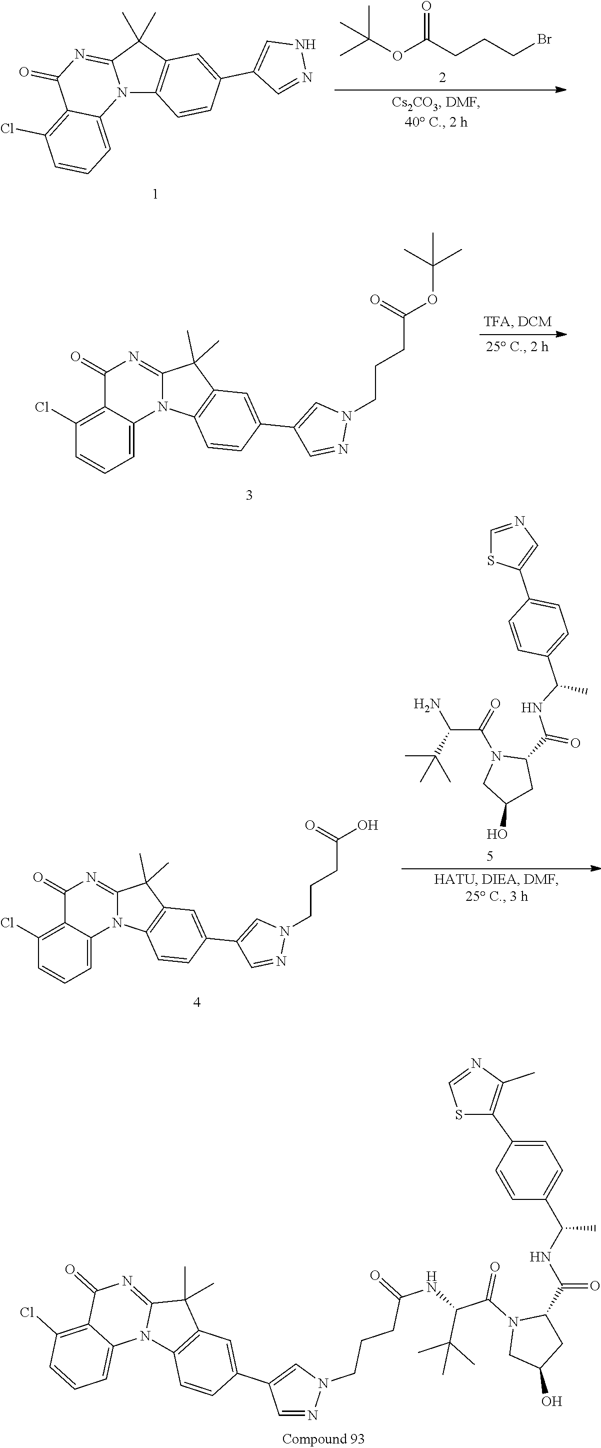

### Synthesis of tert-butyl 4-(4-(4-chloro-7,7-dimethyl-5-oxo-5,7-dihydroindolo[1,2-a]quinazolin-9-yl)-1H-pyrazol-1-yl)butanoate (Intermediate 3)

To a solution of 4-chloro-7,7-dimethyl-9-(1H-pyrazol-4-yl)indolo[1,2-a]quinazolin-5 (7H)-one (40.0 mg, 1 eq, 110 μmol) in DMF (1 mL) was added t-Butyl4-bromobutanoate (29.5 mg, 1.2 eq, 132 μmol) and Cesium carbonate (71.8 mg, 2 eq, 220 μmol). The reaction was stirred at 40° C. for 2 hours. LCMS indicated completion of reaction. The reaction was poured into water (20 mL) and extracted with ethyl acetate (20 mL×2). The combined organic layers were washed with brine, dried over sodium sulfate, filtered and concentrated under vacuum to give tert-butyl 4-(4-(4-chloro-7,7-dimethyl-5-oxo-5,7-dihydroindolo[1,2-a]quinazolin-9-yl)-1H-pyrazol-1-yl)butanoate (100 mg, 0.19 mmol, 89%, 50% Purity) as a white solid. LC purity (0.1% FA): 97% (UV at 254 nm)/MS: 505.3 [M+H]; Retention time: 2.14 min.

### Synthesis of 4-(4-(4-chloro-7,7-dimethyl-5-oxo-5,7-dihydroindolo[1,2-a]quinazolin-9-yl)-1H-pyrazol-1-yl)butanoic acid (Intermediate 4)

A mixture of tert-butyl 4-(4-(4-chloro-7,7-dimethyl-5-oxo-5,7-dihydroindolo[1,2-a]quinazolin-9-yl)-1H-pyrazol-1-yl)butanoate (100 mg, 1 eq, 198 μmol) in TFA (0.3 mL) and DCM (1 mL) was stirred at 30° C. for 2 hours. LCMS indicated completion of reaction. The reaction was concentrated under vacuum to give 4-(4-(4-chloro-7,7-dimethyl-5-oxo-5,7-dihydroindolo[1,2-a]quinazolin-9-yl)-1H-pyrazol-1-yl)butanoic acid (80.0 mg, 0.16 mmol, 82%) as a yellow solid. LC purity (0.1% FA): 91% (UV at 254 nm)/MS: 449.2 [M+H]; Retention time: 1.72 min.

### Synthesis of (2S,4R)-1-((S)-2-(4-(4-(4-chloro-7,7-dimethyl-5-oxo-5,7-dihydroindolo[1,2-a]quinazolin-9-yl)-1H-pyrazol-1-yl)butanamido)-3,3-dimethylbutanoyl)-4-hydroxy-N—((S)-1-(4-(4-methylthiazol-5-yl)phenyl)ethyl)pyrrolidine-2-carboxamide (Compound 93)

To a solution of 4-(4-(4-chloro-7,7-dimethyl-5-oxo-5,7-dihydroindolo[1,2-a]quinazolin-9-yl)-1H-pyrazol-1-yl)butanoic acid (70.0 mg, 1 eq, 156 μmol) in DMF (3 mL) was added (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (83.2 mg, 1.2 eq, 187 μmol), HATU (71.2 mg, 1.2 eq, 187 μmol) and DIEA (60.5 mg, 3 eq, 468 μmol). The reaction was stirred at 20° C. for 16 hours. LCMS indicated completion of reaction. The reaction was poured into water (20 mL) and extracted with ethyl acetate (20 mL×2). The combined organic layers were washed with brine, dried over sodium sulfate, filtered and purified by prep-HPLC, eluted with MeCN in H₂O (0.1% FA) to afford (2S,4R)-1-((S)-2-(4-(4-(4-chloro-7,7-dimethyl-5-oxo-5,7-dihydroindolo[1,2-a]quinazolin-9-yl)-1H-pyrazol-1-yl) butanamido)-3,3-dimethylbutanoyl)-4-hydroxy-N—((S)-1-(4-(4-methylthiazol-5-yl)phenyl)ethyl)pyrrolidine-2-carboxamide (2.35 mg, 2.26 μmol, 1.45%) as a white solid. LC purity (0.1% FA): 84.11% (UV at 254 nm)/MS: 875.4 [M+H]; Retention time: 1.600 min. ¹H NMR (400 MHz, DMSO) δ 8.98 (s, 1H), 8.43-8.49 (d, J=8.8 Hz, 1H), 8.35-8.39 (d, J=8.0 Hz, 1H), 8.28 (s, 1H), 8.13-8.21 (d, J=8.8 Hz, 1H), 8.01 (s, 1H), 7.96 (s, 1H), 7.93-7.99 (d, J=9.6 Hz, 1H), 7.83-7.89 (t, J=8.0 Hz, 1H), 7.68-7.73 (d, J=8.8 Hz, 1H), 7.64-7.68 (d, J=8.0 Hz, 1H), 7.43-7.49 (d, J=8.0 Hz, 2H), 7.38-7.45 (d, J=8.0 Hz, 2H), 5.10-5.16 (d, J=3.2 Hz, 1H), 4.92-4.99 (t, J=7.2 Hz, 1H), 4.53-4.59 (d, J=9.2 Hz, 1H), 4.44-4.49 (t, J=7.6 Hz, 1H), 4.29 (s, 1H), 4.14-4.19 (t, J=7.2 Hz, 2H), 3.63 (s, 2H), 3.10-3.08 (m, 1H), 2.45 (s, 3H), 2.07-1.99 (m, 4H), 1.54 (s, 6H), 1.37-1.42 (d, J=7.2 Hz, 3H), 1.19-1.26 (t, J=7.2 Hz, 1H), 0.96 (s, 9H).

The examples in the table below were prepared according to the same method as Compound B93 using appropriate starting materials

| | Calcd. | Found | |
|---|---|---|---|
| Compound | (M + H)⁺ | (M + H)⁺ | |
| No. | m/z | m/z | ¹H NMR |
| B92 | 861.3 | 861.4 | ¹H NMR (400 MHz, CD3OD) δ 8.86 (s, 1H), |
| | | | 8.56-8.53 (m, 1H), 8.18-8.08 (m, 2H), 8.01 (s, |
| | | | 1H), 7.89 (s, 1H), 7.70-7.59 (m, 3H), 7.43-7.35 |
| | | | (m, 4H), 3.86-3.84 (m, 2H), 3.73 (s, 3H), 3.13 |
| | | | (s, 1H), 2.86 (s, 1H), 2.47 (s, 3H), 1.61 (s, 6H), |
| | | | 1.58-1.54 (m, 2H), 1.46-1.42 (m, 3H), 1.12 (s, |
| | | | 1H), 1.06 (s, 2H), 0.95 (s, 9H). |
| B83 | 875.3 | 875.1 | ¹H NMR (400 MHz, CD3OD) δ 8.86 (s, 1H), |
| | | | 8.57-8.55 (d, J = 8.7 Hz, 1H), 8.25-8.23 (t, J = |
| | | | 5.5 Hz, 2H), 8.04 (s, 1H), 7.92-7.88 (m, 1H), |
| | | | 7.69-7.61 (m, 4H), 7.44-7.37 (m, 4H), 7.28-7.27 |
| | | | (m, 1H), 4.97-4.91 (m, 1H), 4.63 (s, 1H), 4.58- |
| | | | 4.56 (d, J = 7.9 Hz, 1H), 4.44 (s, 1H), 4.25-4.22 |
| | | | (m, 2H), 3.94-3.91 (d, J = 10.5 Hz, 1H), 3.78- |
| | | | 3.74 (m, 1H), 2.46 (s, 3H), 2.35-2.31 (m, 3H), |
| | | | 2.23-2.16 (m, 3H), 1.98-1.95 (m, 1H), 1.62 (s, |
| | | | 6H), 1.45-1.43 (d, J = 7.0 Hz, 3H), 1.06 (s, 9H). |
| B101 | 861.3 | 861.4 | ¹H NMR (400 MHz, CD3OD) δ 8.99 (s, 1H), |
| | | | 8.45-8.41 (m, 1H), 8.13-8.07 (m, 2H), 7.95 (s, |
| | | | 1H), 7.88-7.82 (m, 3H), 7.69-7.65 (m, 1H), |
| | | | 7.44-7.38 (m, 4H), 5.01-4.96 (m, 1H), 4.58-4.46 |
| | | | (m, 3H), 3.89-3.85 (m, 1H), 3.76-3.70 (m, |
| | | | 1H), 3.65-3.55 (m, 1H), 3.02-2.95 (m, 1H), |
| | | | 2.93-2.77 (m, 2H), 2.47 (s, 3H), 2.29 (s, 1H), |
| | | | 2.21-2.13 (m, 1H), 1.99-1.91 (m, 1H), 1.65 (s, |
| | | | 6H), 1.50-1.42 (m, 3H), 0.95 (s, 9H). |

### Compound B86. (2S,4R)-1-((S)-2-(4-(6-(4-(4-chloro-7,7-dimethyl-5-oxo-5,7-dihydroindolo[1,2-a]quinazolin-10-yl)piperidin-1-yl)spiro[3.3]heptan-2-yl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

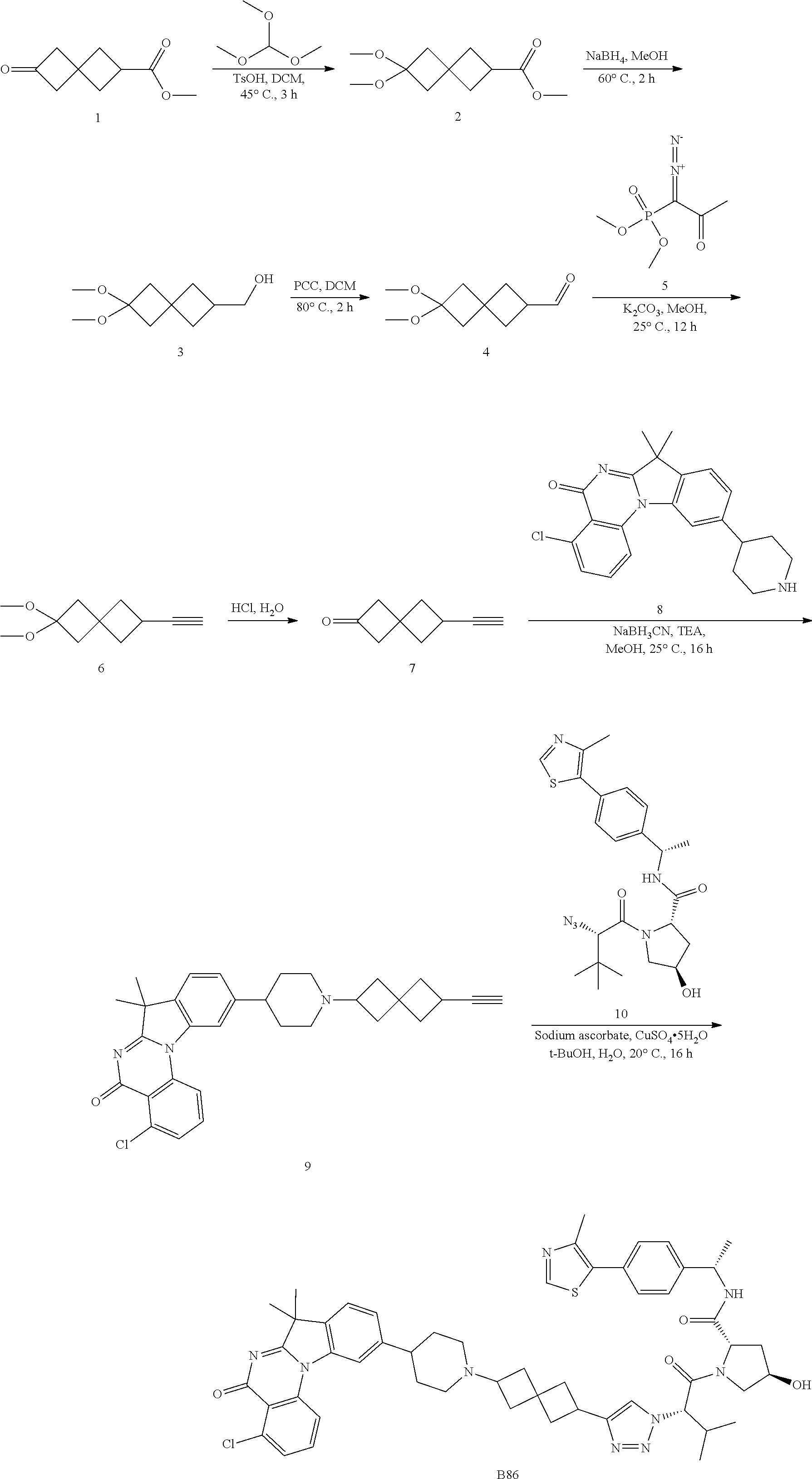

### Synthesis of methyl 6,6-dimethoxyspiro[3.3]heptane-2-carboxylate (Intermediate 2)

To a solution of methyl 6-oxospiro[3.3]heptane-2-carboxylate (3.00 g, 1 eq, 17.8 mmol) in DCM (30 mL) were added p-Toluenesulfonic acid (123 mg, 110 μL, 0.04 eq, 713 μmol), trimethoxymethane (3.79 g, 2 eq, 35.7 mmol), The mixture was stirred at 45° C. for 3 hour. TLC indicated completion of reaction. The reaction mixture was diluted with water (20 mL), extracted with DCM (20 mL×3). The combined organic layers were dried over anhydrous sodium sulfate, filtered and concentrated under vacuum. The residue was purified by silica gel column chromatography (100-200 mesh silica gel, 3% of EA in PE) to afford methyl 6,6-dimethoxyspiro[3.3]heptane-2-carboxylate (3.60 g, 15 mmol, 85%, 90% Purity) as colorless oil. H NMR (400 MHz, CDCl₃) δ 3.67 (s, 3H), 3.12 (s, 6H), 3.01-3.05 (m, 1H), 2.31-2.34 (m, 2H), 2.23-2.27 (m, 4H), 2.15 (s, 1H).

### Synthesis of (6,6-dimethoxyspiro[3.3]heptan-2-yl) methanol (Intermediate 3)

To a solution of methyl 6,6-dimethoxyspiro[3.3]heptane-2-carboxylate (3.60 g, 1 eq, 16.8 mmol) in MeOH (30 mL) were added NaBH₄ (12.7 g, 20 eq, 336 mmol), The mixture was stirred at 60° C. for 16 hour. TLC indicated completion of reaction. The reaction mixture was diluted with water (20 mL), extracted with DCM (20 mL×3). The combined organic layers were dried over anhydrous sodium sulfate, filtered and concentrated under vacuum. The resulting residue was purified by silica gel column chromatography (100-200 mesh silica gel, 15% of EA in PE) to afford (6,6-dimethoxyspiro[3.3]heptan-2-yl) methanol (1.50 g, 7.4 mmol, 44%, 92% Purity) as colorless oil. ¹H NMR (400 MHz, DMSO) δ 4.32-4.36 (m, 1H), 3.31 (d, J=8 Hz, 2H), 3.00 (s, 6H), 2.20-2.23 (m, 1H), 2.11 (s, 2H), 2.01 (s, 2H), 1.93-1.96 (m, 2H), 1.70-1.75 (m, 2H).

### Synthesis of 6,6-dimethoxyspiro[3.3]heptane-2-carbaldehyde (Intermediate 4)

To a solution of (6,6-dimethoxyspiro[3.3]heptan-2-yl) methanol (200 mg, 1 Eq, 1.07 mmol) in DCM (6.00 mL) were added PCC (463 mg, 2 Eq, 2.15 mmol), The mixture was stirred at 25° C. for 2 hour. TLC indicated completion of reaction. The resulting reaction mixture was diluted with water (20 mL), extracted with DCM (20 mL×3). The combined organic layers were dried over anhydrous sodium sulfate, filtered and concentrated under vacuum. The residue was purified by silica gel column chromatography (100-200 mesh silica gel, 10% of EA in PE) to afford 6,6-dimethoxyspiro[3.3]heptane-2-carbaldehyde (100 mg, 0.50 mmol, 47%, 92% Purity) as colorless oil. H NMR (400 MHz, DMSO) δ 9.62-9.64 (m, 1H), 3.07-3.11 (m, 1H), 3.00 (s, 6H), 2.20-2.23 (m, 1H), 2.10-2.21 (m, 6H), 2.01 (s, 2H).

### Synthesis of 6-ethynyl-2,2-dimethoxyspiro[3.3]heptane (Intermediate 6)

To a solution of 6,6-dimethoxyspiro[3.3]heptane-2-carbaldehyde (500 mg, 1 eq, 2.71 mmol) in MeOH (2 mL) were added K₂CO₃ (1.13 g, 3 eq, 8.14 mmol), dimethyl (1-diazo-2-oxopropyl)phosphonate (1.56 g, 3 eq, 8.14 mmol), The mixture was stirred at 25° C. for 2 hour. TLC indicated completion of reaction. The reaction mixture was diluted with water (20 mL), extracted with DCM (20 mL×3). The combined organic layers were dried over anhydrous sodium sulfate, filtered and concentrated under vacuum. The resulting residue was purified by silica gel column chromatography (100-200 mesh silica gel, 2% of EA in PE) to afford 6-ethynyl-2,2-dimethoxyspiro[3.3]heptane (100 mg, 0.44 mmol, 16%, 80% Purity) as colorless oil. ¹H NMR (400 MHz, DMSO) δ 3.00 (s, 6H), 2.93-2.94 (m, 1H), 2.88-2.89 (m, 1H), 2.27-2.30 (m, 2H), 2.12-2.13 (m, 2H), 2.01-2.06 (m, 4H).

### Synthesis of 6-ethynyl-2,2-dimethoxyspiro[3.3]heptane (Intermediate 7)

To a solution of 6-ethynyl-2,2-dimethoxyspiro[3.3]heptane (100 mg, 1 Eq, 0.55 mmol) in HCl/EA (2.00 mL). The mixture was stirred at 25° C. for 2 hour. TLC indicated completion of reaction. The reaction mixture was diluted with water (20 mL), extracted with DCM (20 mL×3). The combined organic layers were dried over anhydrous sodium sulfate, filtered and concentrated under vacuum, The resulting crude was used directly in the next.

### Synthesis of 9-(1-(((1s,4s)-4-aminocyclohexyl)methyl)piperidin-4-yl)-4-chloro-7,7-dimethylindolo[1,2-a]quinazolin-5 (7H)-one (Intermediate 9)

To a solution of 6-ethynylspiro[3.3]heptan-2-one (35.0 mg, 1 eq, 261 μmol) in MeOH (2 mL) was added TEA (79.2 mg, 109 μL, 3 eq, 783 μmol), NaCNBH₃ (32.8 mg, 2 eq, 522 μmol) and 4-chloro-7,7-dimethyl-10-(piperidin-4-yl)indolo[1,2-a]quinazolin-5 (7H)-one (149 mg, 1.5 eq, 391 μmol), and the reaction was stirred at 25° C. for 16 hours. LCMS showed the reaction was completed. The reaction was diluted with water, and extracted with EA, washed three times with saturated saline and then dried with anhydrous sodium sulfate concentrate, the organic layer was concentrated in vacuum, the residue was purified by flash silica gel column chromatography with DCM/MeOH=20/1 to give 4-chloro-10-(1-(6-ethynylspiro[3.3]heptan-2-yl)piperidin-4-yl)-7,7-dimethylindolo[1,2-a]quinazolin-5 (7H)-one (30.0 mg, 60.2 μmol, 23.1%) as a yellow solid. LC purity (0.1% FA): 100% (UV at 254 nm)/MS: 498.2 [M+H]; Retention time: 1.23 min.

### Synthesis of (2S,4R)-1-((S)-2-(4-(6-(4-(4-chloro-7,7-dimethyl-5-oxo-5,7-dihydroindolo[1,2-a]quinazolin-10-yl)piperidin-1-yl)spiro[3.3]heptan-2-yl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide (Compound 86)

To a solution of 4-chloro-10-(1-(6-ethynylspiro[3.3]heptan-2-yl)piperidin-4-yl)-7,7-dimethylindolo[1,2-a]quinazolin-5 (7H)-one (30.0 mg, 1 eq, 60.2 μmol) in 1:1 mixture of t-BuOH (1 mL) and Water (1 mL) was added (2S,4R)-1-((S)-2-azido-3,3-dimethylbutanoyl)-4-hydroxy-N—((S)-1-(4-(4-methylthiazol-5-yl)phenyl)ethyl)pyrrolidine-2-carboxamide (34.0 mg, 1.2 eq, 72.3 μmol). Then Copper (II) SulfatePentahydrate (15.0 mg, 14.6 μL, 1 eq, 60.2 μmol) was added, followed by Sodium 2-(1,2-dihydroxy-ethyl)-4-hydroxy-5-oxo-2,5-dihydro-furan-3-olate (5.97 mg, 0.5 eq, 30.1 μmol). The reaction was stirred at 30° C. for 16 hours. LCMS showed the reaction was completed. The reaction was diluted with water, and extracted with EA, washed three times with brine and then dried with anhydrous sodium sulfate, the organic layer was concentrated in vacuum, the residue was purified by prep-HPLC, eluted with CH₃CN in H₂O (0.1% FA) to give (2S,4R)-1-((S)-2-(4-(6-(4-(4-chloro-7,7-dimethyl-5-oxo-5,7-dihydroindolo[1,2-a]quinazolin-10-yl)piperidin-1-yl)spiro[3.3]heptan-2-yl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide (3.10 mg, 3.14 μmol, 5.21%) as a white solid. LC purity (0.1% TFA): 100% (UV at 254 nm)/MS: 484.9 [M/2+H]; Retention time: 1.40 min. ¹H NMR (400 MHz, DMSO-d₆) δ 8.87 (s, 1H), 8.42-8.44 (d, J=8 Hz, 1H), 8.05-8.07 (d, J=8 Hz, 1H), 7.98 (s, 1H), 7.84-7.88 (m, 1H), 7.66 7.68 (d, J=8 Hz, 1H), 7.60-7.62 (d, J=8 Hz, 1H), 7.33-7.46 (m, 5H), 5.50 (s, 1H), 5.02-5.07 (m, 1H), 4.52-4.56 (m, 1H), 4.45 (s, 1H), 3.84-3.87 (m, 1H), 3.74-3.76 (d, J=8 Hz, 1H), 3.56-3.61 (m, 1H), 3.38-3.48 (m, 3H), 2.97-3.02 (m, 1H), 2.58-2.65 (m, 4H), 2.47-2.48 (d, J=4 Hz, 3H), 2.34-2.43 (m, 4H), 2.12-2.28 (m, 5H), 1.93-2.03 (m, 3H), 1.64-1.65 (d, J=4 Hz, 1H), 1.59 (s, 6H), 1.53-1.55 (d, J=8 Hz, 2H), 1.06 (s, 9H).

### Compound B89 and Compound B90. (2S,4R)-1-(2-(3-(4-(4-(4-chloro-7,7-dimethyl-5-oxo-5,7-dihydroindolo[1,2-a]quinazolin-9-yl)piperidine-1-carbonyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide

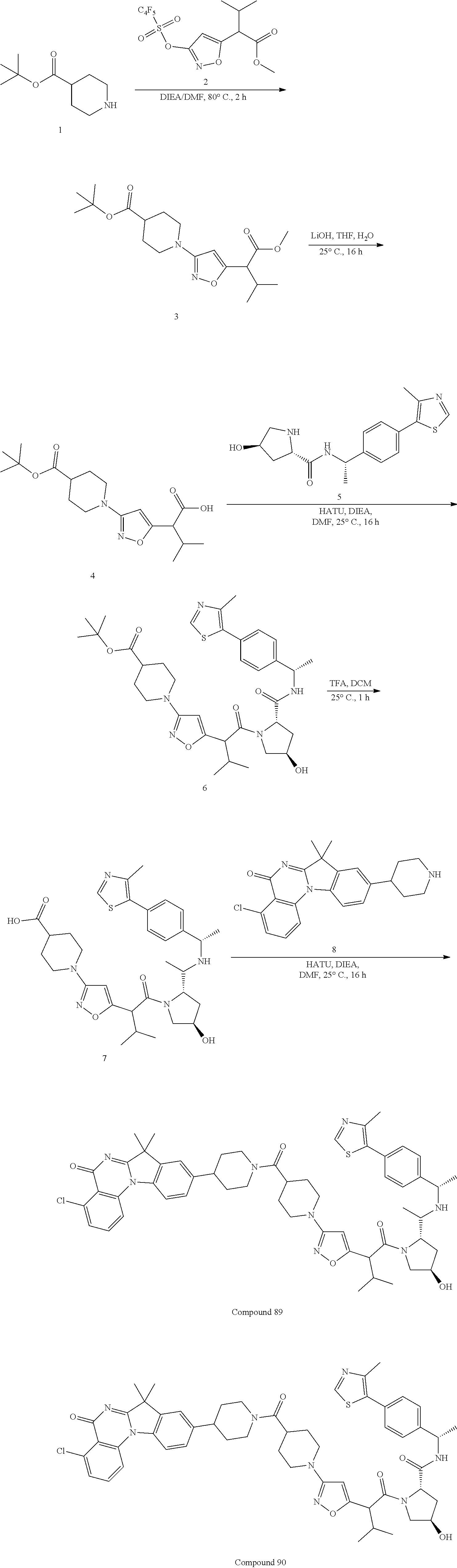

### Synthesis of tert-butyl 1-(5-(1-methoxy-3-methyl-1-oxobutan-2-yl)isoxazol-3-yl)piperidine-4-carboxylate (Intermediate 3)

To a solution of tert-butyl piperidine-4-carboxylate (150 mg, 3 eq, 810 μmol) in DMF (5 mL) was added DIEA (175 mg, 5 eq, 1.35 mmol) and methyl 3-methyl-2-(3-(((perfluorobutyl) sulfonyl)oxy)isoxazol-5-yl)butanoate (130 mg, 1 eq, 270 μmol). The mixture was stirred at 80° C. for 16 hours. LCMS indicated completion of reaction. The reaction was poured into H₂O (30 mL) and extracted with ethyl acetate (30 mL×2). The combined organic layers were washed with brine (30 mL) and dried over sodium sulfate, filtered and concentrated. The resulting residue was purified by column chromatography (100-200 mesh silica gel 30% of EA in PE) to afford tert-butyl 1-(5-(1-methoxy-3-methyl-1-oxobutan-2-yl)isoxazol-3-yl)piperidine-4-carboxylate (70.0 mg, 191 μmol, 70.7%) as a yellow solid. LC purity (0.1% FA): 49.17% (UV at 254 nm)/MS: 367.2 [M+H]; Retention time: 1.72 min.

### Synthesis of 2-(3-(4-(tert-butoxycarbonyl)piperidin-1-yl)isoxazol-5-yl)-3-methylbutanoic acid (Intermediate 4)

To a solution of tert-butyl 1-(5-(1-methoxy-3-methyl-1-oxobutan-2-yl)isoxazol-3-yl)piperidine-4-carboxylate (70.0 mg, 1 eq, 191 μmol) in THF (3 mL) and Water (1 mL) was added LiOH (45.7 mg, 10 eq, 1.91 mmol). The mixture was stirred at 25° C. for 1 hour. LCMS indicated completion of reaction. The reaction was adjusted PH to 4. The reaction was poured into H₂O (30 mL) and extracted with ethyl acetate (30 mL×2). The combined organic layers were washed with brine (30 mL) and dried over sodium sulfate, filtered and concentrated to afford 2-(3-(4-(tert-butoxycarbonyl)piperidin-1-yl)isoxazol-5-yl)-3-methylbutanoic acid (60.0 mg, 170 μmol, 89.1%) as a yellow solid. LC purity (0.1% FA): 64.35% (UV at 254 nm)/MS: 352.2 [M+H]; Retention time: 1.58 min.

### Synthesis of tert-butyl 1-(5-(1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)piperidine-4-carboxylate (Intermediate 6)

To a solution of 2-(3-(4-(tert-butoxycarbonyl)piperidin-1-yl)isoxazol-5-yl)-3-methylbutanoic acid (70.0 mg, 1 eq, 199 μmol) in DMF (3 mL) was added (2S,4R)-4-hydroxy-N—((S)-1-(4-(4-methylthiazol-5-yl)phenyl)ethyl)pyrrolidine-2-carboxamide (98.7 mg, 1.5 eq, 298 μmol), DIEA (128 mg, 173 μL, 5 eq, 993 μmol) and HATU (151 mg, 2 eq, 397 μmol). The mixture was stirred at 25° C. for 16 hours. LCMS indicated completion of reaction. The reaction was poured into H₂O (30 mL) and extracted with ethyl acetate (30 mL×2). The combined organic layers were washed with brine (30 mL) and dried over sodium sulfate, filtered and concentrated. The resulting residue was purified by column chromatography (100-200 mesh silica gel, 5% of MeOH in DCM) to afford tert-butyl 1-(5-(1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)piperidine-4-carboxylate (50.0 mg, 75.1 μmol, 37.8%) as a yellow solid. LC purity (0.1% FA): 23.22% (UV at 254 nm)/MS: 666.4 [M+H]; Retention time: 1.61 min.

### Synthesis of 1-(5-(1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)piperidine-4-carboxylic acid (Intermediate 7)

To a solution of tert-butyl 1-(5-(1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)piperidine-4-carboxylate (100.0 mg, 1 eq, 150.2 μmol) in DCM (3 mL) and TFA (1 mL). The mixture was stirred at 25° C. for 1 hour. LCMS indicated completion of reaction. The reaction was concentrated under vacuum to afford 1-(5-(1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)piperidine-4-carboxylic acid (80.0 mg, 131 μmol, 87.4%) as a yellow solid. LC purity (0.1% FA): 78% (UV at 254 nm)/MS: 610.3 [M+H]; Retention time: 0.93 min.

### Synthesis of (2S,4R)-1-(2-(3-(4-(4-(4-chloro-7,7-dimethyl-5-oxo-5,7-dihydroindolo[1,2-a]quinazolin-9-yl)piperidine-1-carbonyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 89 and Compound 90)

To a solution of 1-(5-(1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)piperidine-4-carboxylic acid (45.0 mg, 1 eq, 73.8 μmol) in DMF (3 mL) was added HATU (56.1 mg, 2 eq, 148 μmol), 4-chloro-7,7-dimethyl-9-(piperidin-4-yl)indolo[1,2-a]quinazolin-5 (7H)-one (42.1 mg, 1.5 eq, 111 μmol) and DIEA (47.7 mg, 64.3 μL, 5 eq, 369 μmol). The mixture was stirred at 25° C. for 16 hours. LCMS indicated completion of reaction. The reaction was purified by prep-HPLC, eluted with MeCN in H₂O (0.1% FA) to afford (2S,4R)-1-(2-(3-(4-(4-(4-chloro-7,7-dimethyl-5-oxo-5,7-dihydroindolo[1,2-a]quinazolin-9-yl)piperidine-1-carbonyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (4.84 mg, 4.76 μmol, 6.46%) as a white solid, LC purity (0.1% FA): 85.11% (UV at 254 nm)/MS: 971.2 [M+1]; Retention time: 1.59 min. ¹H NMR (400 MHz, DMSO) δ 8.95-8.99 (m, 1H), 8.40-8.42 (m, 1H), 8.27-8.12 (m, 1H), 8.07 (m, 1H), 7.82 (m, 1H), 7.68-7.59 (m, 2H), 7.50-7.29 (m, 6H), 6.16 (m, 1H), 5.12-4.85 (m, 2H), 4.64-4.40 (m, 2H), 4.21-4.24 (m, 2H), 3.71-3.52 (m, 4H), 3.15 (s, 2H), 2.92-2.80 (m, 4H), 2.65-2.69 (m, 1H), 2.45 (s, 3H), 2.29-2.37 (m, 4H), 1.92-1.79 (m, 3H), 1.66 (s, 4H), 1.52 (s, 6H), 1.36 (m, 2H), 0.99-0.78 (m, 6H) and (2S,4R)-1-(2-(3-(4-(4-(4-chloro-7,7-dimethyl-5-oxo-5,7-dihydroindolo[1,2-a]quinazolin-9-yl)piperidine-1-carbonyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (6.87 mg, 6.32 μmol, 8.57%) as a white solid. LC purity (0.1% FA): 91.88% (UV at 254 nm)/MS: 971.2 [M+1]; Retention time: 1.60 min. ¹H NMR (400 MHz, DMSO-d₆) δ 8.98-9.04 (d, J=4.0 Hz, 1H), 8.40-8.44 (m, 2H), 8.02-8.11 (m, 1H), 7.76-7.87 (m, 1H), 7.58-7.69 (m, 2H), 7.28-7.54 (m, 6H), 6.15 (s, 1H), 5.07-5.12 (t, J=21.2 Hz, 1H), 4.89-4.97 (m, 1H), 4.56-4.68 (m, 1H), 4.31-4.43 (m, 1H), 4.29 (s, 1H), 4.15 (s, 1H), 3.61-3.75 (m, 3H), 3.51-3.60 (m, 1H), 3.41-3.49 (m, 1H), 3.16 (s, 1H), 2.89-2.99 (d, J=12.8 Hz, 4H), 2.60-2.69 (m, 1H), 2.45-2.49 (d, J=4.4 Hz, 3H), 2.21-2.36 (m, 1H), 2.02 (s, 1H), 1.78-1.95 (m, 3H), 1.67 (s, 4H), 1.52 (s, 6H), 1.40-1.51 (m, 1H), 1.32-1.45 (m, 3H), 0.96-1.03 (d, J=6.4 Hz, 3H), 0.71-0.88 (m, 3H).

### Compound B102. (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-N—((R)-1-(4-ethynylphenyl)-2-morpholinoethyl)-4-hydroxypyrrolidine-2-carboxamide

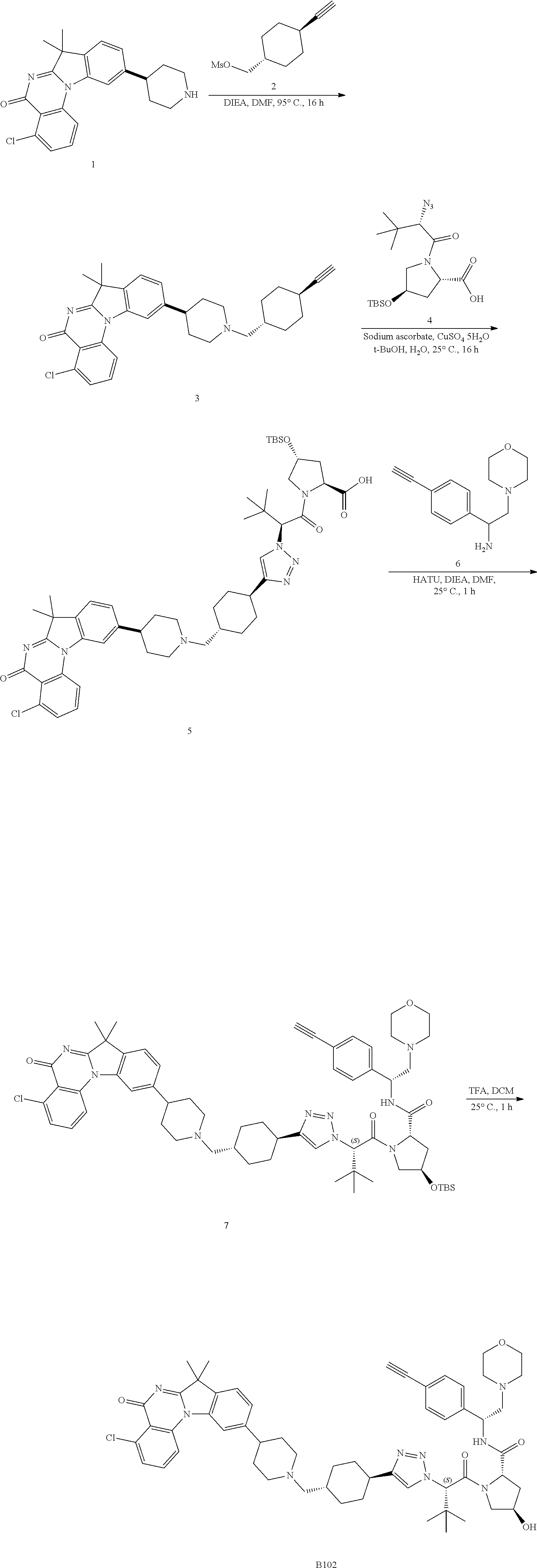

### Synthesis of(S)-4-chloro-7,7-dimethyl-10-(1-(5-methylhept-6-yn-1-yl)piperidin-4-yl)indolo[1,2-a]quinazolin-5 (7H)-one

To a solution of ((1r,4r)-4-ethynylcyclohexyl)methyl methanesulfonate (1.14 g, 2 eq, 5.24 mmol) in DMF (5 mL) was added DIEA (2.15 mL, 5 eq, 12.6 mmol) and 4-chloro-7,7-dimethyl-10-(piperidin-4-yl)indolo[1,2-a]quinazolin-5 (7H)-one (1 g, 1 eq, 2.62 mmol). The reaction was stirred 95° C. for 16 hours. LCMS indicated completion of reaction. The mixture was poured into water (20 mL), extracted with EA (20 mL×3). The combined organic layers were washed with brine (30 mL), dried over sodium sulfate, filtered and concentrated under vacuum. The resulting residue was purified by column (20% EA in PE) to get(S)-4-chloro-7,7-dimethyl-10-(1-(5-methylhept-6-yn-1-yl)piperidin-4-yl)indolo[1,2-a]quinazolin-5 (7H)-one (300 mg, 615 μmol, 46.7%) as a yellow solid. LC purity (0.1% FA): 86.00% (UV at 254 nm)/MS: 500.4 [M+H]; Retention time: 0.72 min.

### Synthesis of (2S,4R)-4-((tert-butyldimethylsilyl)oxy)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)pyrrolidine-2-carboxylic acid

To a solution of 4-chloro-10-(1-(((1r,4r)-4-ethynylcyclohexyl)methyl)piperidin-4-yl)-7,7-dimethylindolo[1,2-a]quinazolin-5 (7H)-one (600 mg, 1 eq, 1.20 mmol) in H₂O (5 mL) and t-BuOH (5 mL) was added copper (II) sulfate pentahydrate (300 mg, 292 μL, 1 eq, 1.20 mmol) and sodium 2-(1,2-dihydroxy-ethyl)-4-hydroxy-5-oxo-2,5-dihydro-furan-3-olate (238 mg, 1 eq, 1.20 mmol). The reaction was stirred 25° C. for 16 hours. LCMS indicated completion of reaction. The mixture was poured into water (20 mL), extracted with MeOH/DCM=1/10 (100 mL). The organic layer was washed with brine (30 mL), dried over sodium sulfate, filtered and concentrated under vacuum. The resulting residue was purified by column (100-200 mesh silica gel, 10% of MeOH in DCM) to afford (2S,4R)-4-((tert-butyldimethylsilyl)oxy)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)pyrrolidine-2-carboxylic acid (600 mg, 678 μmol, 56.5%) as a yellow solid. LC purity (0.1% FA): 37.58% (UV at 254 nm)/MS: 884.4 [M+H]; Retention time: 1.51 min.

### Synthesis of (2S,4R)-4-((tert-butyldimethylsilyl)oxy)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-N—((R)-1-(4-ethynylphenyl)-2-morpholinoethyl)pyrrolidine-2-carboxamide

To a solution of (2S,4R)-4-((tert-butyldimethylsilyl)oxy)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)pyrrolidine-2-carboxylic acid (115 mg, 1 eq, 130 μmol) in DMF (1 mL) was added DIEA (101 mg, 136 μL, 6 eq, 782 μmol) and HATU (99.1 mg, 2 eq, 261 μmol). The mixture was stirred at 25° C. for 10 mins, then (R)-1-(4-ethynylphenyl)-2-morpholinoethan-1-amine (30.0 mg, 1 eq, 130 μmol) was added and the reaction was stirred at 25° C. for 1 hour. LCMS indicated completion of reaction. The mixture was poured into water (20 mL), extracted with ethyl acetate (20 mL×3). The combined organic layers were washed with brine (50 mL), dried over sodium sulfate, filtered and concentrated under vacuum. The resulting residue was purified by silica gel chromatography (100-200 mesh silica gel, 6% of MeOH in DCM) to afford (2S,4R)-4-((tert-butyldimethylsilyl)oxy)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-N—((R)-1-(4-ethynylphenyl)-2-morpholinoethyl)pyrrolidine-2-carboxamide (45.0 mg, 33 μmol, 25%, 80% purity) as a yellow oil. LC purity (0.1% FA): 79% (UV at 254 nm)/MS: 549.0 [1/2M+H]; Retention time: 1.5 min.

### Synthesis of (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-N—((R)-1-(4-ethynylphenyl)-2-morpholinoethyl)-4-hydroxypyrrolidine-2-carboxamide

A mixture of (2S,4R)-4-((tert-butyldimethylsilyl)oxy)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-N—((R)-1-(4-ethynylphenyl)-2-morpholinoethyl)pyrrolidine-2-carboxamide (40.0 mg, 1 eq, 36.5 μmol) in DCM (1 mL) and TFA (3 mL) was stirred at 25° C. for 1 hour. LCMS indicated completion of reaction. The mixture was concentrated under vacuum and the resulting residue was purified by prep-HPLC (Waters 3767/Qda, Column: XBridge XBridge C18, 19*250 mm, 10 um; Mobile Phase A: 10 mmol/L NH₄HCO₃/H₂O, B:ACN; flow rate: 20 ml/min; gradient: 54-64%; Retention Time: 8.4-9.4 min of 17 min) to afford (2S,4R)-1-((S)-2-(4-((1r,4S)-4-((4-(4-chloro-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-N—((R)-1-(4-ethynylphenyl)-2-morpholinoethyl)-4-hydroxypyrrolidine-2-carboxamide (5.33 mg, 5.03 μmol, 13.8%, 92.76% Purity) as a white solid. LC purity (0.1% FA): 97.70% (UV at 254 nm)/MS: 465.4 [1/2M+H]; Retention time: 1.592 min. ¹H NMR (400 MHz, MeOD-d₄) δ 8.44 (d, J=8.6 Hz, 1H), 1.97-8.02 (m, 2H), 7.88 (t, J=8.3 Hz, 1H), 7.66 (d, J=7.9 Hz, 1H), 7.57 (d, J=7.8 Hz, 1H), 7.46-7.19 (m, 5H), 5.04-5.08 (m, 1H), 4.54-4.58 (m, 3H), 4.42-4.46 (m, 1H), 3.72-3.76 (m, 4H), 3.44-3.45 (m, 1H), 3.14-3.17 (m, 2H), 2.86-2.70 (m, 3H), 2.64-2.49 (m, 4H), 2.32-2.35 (m, 2H), 2.26-2.07 (m, 5H), 2.04-1.89 (m, 8H), 1.59 (s, 6H), 1.56-1.45 (m, 2H), 1.28-1.32 (m, 2H), 1.12-1.22 (m, 2H), 1.05-1.07 (m, 9H).

### Compound B104. (2S,4R)—N—((S)-1-(4-(4-(3-(2-(2-(4-(4-chloro-7,7-dimethyl-5-oxo-5,7-dihydroindolo[1,2-a]quinazolin-10-yl)piperidin-1-yl)ethoxy) ethoxy)prop-1-yn-1-yl)thiazol-5-yl)phenyl)ethyl)-4-hydroxy-1-(3-methyl-2-(3-methylisoxazol-5-yl)butanoyl)pyrrolidine-2-carboxamide

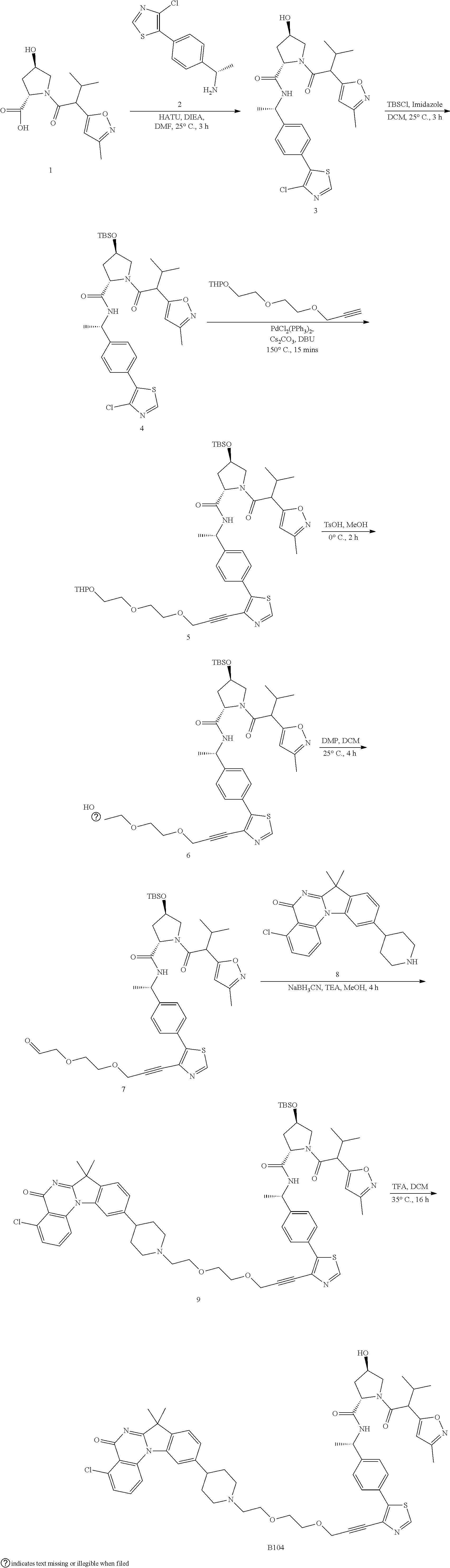

### Synthesis of (2S,4R)—N-(1-(4-(4-chlorothiazol-5-yl)phenyl)ethyl)-4-hydroxy-1-((R)-3-methyl-2-(3-methylisoxazol-5-yl)butanoyl)pyrrolidine-2-carboxamide

To a solution of (2S,4R)-4-hydroxy-1-(3-methyl-2-(3-methylisoxazol-5-yl)butanoyl)pyrrolidine-2-carboxylic acid (205 mg, 1.1 eq, 691 μmol) in DMF (4 mL) were added DIEA (487 mg, 657 μL, 6 eq, 3.77 mmol), HATU (287 mg, 1.2 eq, 754 μmol) and(S)-1-(4-(4-chlorothiazol-5-yl)phenyl) ethan-1-amine (150 mg, 1 eq, 628 μmol). The mixture was stirred at 25° C. for 3 hours. LCMS indicated completion of reaction. The resulting residue was purified by prep-HPLC (Waters 3767/Qda Column: Sunfire C1819*250 mm, 10 um; Mobile Phase A: 0.1% FA/H₂O, B:ACN; flow rate: 60 ml/min; gradient: 35-45% Retention Time: 7.6 of 16 min) to afford (2S,4R)—N-(1-(4-(4-chlorothiazol-5-yl)phenyl)ethyl)-4-hydroxy-1-((R)-3-methyl-2-(3-methylisoxazol-5-yl)butanoyl)pyrrolidine-2-carboxamide (200 mg, 0.37 mmol, 58%) as a white solid. LC purity (0.1% FA): 97.76% (UV at 254 nm)/MS: 517.2 [M+H]; Retention time: 1.47 min.

### Synthesis of (2S,4R)-4-((tert-butyldimethylsilyl)oxy)-N—((S)-1-(4-(4-chlorothiazol-5-yl)phenyl)ethyl)-1-(3-methyl-2-(3-methylisoxazol-5-yl)butanoyl)pyrrolidine-2-carboxamide

To a solution of (2S,4R)—N—((S)-1-(4-(4-chlorothiazol-5-yl)phenyl)ethyl)-4-hydroxy-1-((R)-3-methyl-2-(3-methylisoxazol-5-yl)butanoyl)pyrrolidine-2-carboxamide (150 mg, 1 eq, 290 μmol) in DCM (4 mL) were added Imidazole (118 mg, 115 μL, 6 eq, 1.74 mmol) and TBSCl (175 mg, 4 eq, 1.160 mmol). The mixture was stirred at 25° C. for 3 hours. LCMS indicated completion of reaction. The mixture was poured into water (20 mL), extracted with DCM (20 mL×3). The combined organic layers were washed with brine (20 mL), dried over sodium sulfate, filtered and concentrated under vacuum. The resulting residue was purified by silica gel chromatography (100-200 mesh silica gel, 35% of EtOAc in PE) to afford (2S,4R)-4-((tert-butyldimethylsilyl)oxy)-N—((S)-1-(4-(4-chlorothiazol-5-yl)phenyl)ethyl)-1-((R)-3-methyl-2-(3-methylisoxazol-5-yl)butanoyl)pyrrolidine-2-carboxamide (170 mg, 0.26 mmol, 88%) as a yellow solid. LC purity (0.1% FA): 96.12% (UV at 254 nm)/MS: 631.2 [M+1]; Retention time: 1.87 min.

### Synthesis of (2S,4R)-4-((tert-butyldimethylsilyl)oxy)-1-((R)-3-methyl-2-(3-methylisoxazol-5-yl) butanoyl)-N-((1S)-1-(4-(4-(3-(2-(2-((tetrahydro-2H-pyran-2-yl)oxy) ethoxy) ethoxy)prop-1-yn-1-yl)thiazol-5-yl)phenyl)ethyl)pyrrolidine-2-carboxamide

To a solution of (2S,4R)-4-((tert-butyldimethylsilyl)oxy)-N—((S)-1-(4-(4-chlorothiazol-5-yl)phenyl)ethyl)-1-((R)-3-methyl-2-(3-methylisoxazol-5-yl)butanoyl)pyrrolidine-2-carboxamide (100 mg, 1 eq, 158 μmol) in DMF (1 mL) were added bis-(triphenylphosphino)-palladous chloride (11.12 mg, 0.1 eq, 15.84 μmol), 2-(2-(2-(prop-2-yn-1-yloxy) ethoxy) ethoxy)tetrahydro-2H-pyran (108.5 mg, 3 eq, 475.2 μmol), Cs₂CO₃ (103.2 mg, 2 eq, 316.8 μmol) and DBU (12.06 mg, 11.94 μL, 0.5 eq, 79.20 μmol). The mixture was stirred at 150° C. for 15 mins under microwave irradiation and N₂. LCMS indicated completion of reaction. The mixture was poured into water (20 mL), extracted with EA (20 mL×3). The combined organic layers were washed with brine (50 mL), dried over sodium sulfate, filtered and concentrated under vacuum. The resulting residue was purified by silica gel column chromatography (100-200 mesh silica gel, 3% of MeOH in DCM) to afford (2S,4R)-4-((tert-butyldimethylsilyl)oxy)-1-((R)-3-methyl-2-(3-methylisoxazol-5-yl)butanoyl)-N-((1S)-1-(4-(4-(3-(2-(2-((tetrahydro-2H-pyran-2-yl)oxy) ethoxy) ethoxy)prop-1-yn-1-yl)thiazol-5-yl)phenyl)ethyl)pyrrolidine-2-carboxamide (160 mg, 97 μmol, 61%, 50% purity) as a yellow solid. LC purity (0.1% FA): 23.5% (UV at 254 nm)/MS: 823.1 [M+1]; Retention time: 2.16 min.

### Synthesis of (2S,4R)-4-((tert-butyldimethylsilyl)oxy)-N—((S)-1-(4-(4-(3-(2-(2-hydroxyethoxy) ethoxy)prop-1-yn-1-yl)thiazol-5-yl)phenyl)ethyl)-1-((R)-3-methyl-2-(3-methylisoxazol-5-yl)butanoyl)pyrrolidine-2-carboxamide

To a solution of (2S,4R)-4-((tert-butyldimethylsilyl)oxy)-1-((R)-3-methyl-2-(3-methylisoxazol-5-yl)butanoyl)-N-((1S)-1-(4-(4-(3-(2-(2-((tetrahydro-2H-pyran-2-yl)oxy) ethoxy) ethoxy)prop-1-yn-1-yl)thiazol-5-yl)phenyl)ethyl)pyrrolidine-2-carboxamide (150 mg, 50% Wt, 1 eq, 91.1 μmol) in MeOH (2 mL) were added p-toluenesulfonic acid (15.7 mg, 14.1 μL, 1 eq, 91.1 μmol). The mixture was stirred at 0° C. for 2 hours. LCMS indicated completion of reaction. The mixture was poured into water (20 mL), extracted with EA (20 mL×3). The combined organic layers were washed with brine (30 mL), dried over sodium sulfate, filtered and concentrated under vacuum. The resulting residue was purified by silica gel column chromatography (100-200 mesh silica gel, 6% of MeOH in DCM) to afford (2S,4R)-4-((tert-butyldimethylsilyl)oxy)-N—((S)-1-(4-(4-(3-(2-(2-hydroxyethoxy) ethoxy)prop-1-yn-1-yl)thiazol-5-yl)phenyl)ethyl)-1-((R)-3-methyl-2-(3-methylisoxazol-5-yl)butanoyl)pyrrolidine-2-carboxamide (24.0 mg, 31 μmol, 34%) as a yellow solid. LC purity (0.1% FA): 94.45% (UV at 254 nm)/MS: 739.4 [M+1]; Retention time: 1.67 min.

### Synthesis of (2S,4R)-4-((tert-butyldimethylsilyl)oxy)-1-((R)-3-methyl-2-(3-methylisoxazol-5-yl) butanoyl)-N—((S)-1-(4-(4-(3-(2-(2-oxoethoxy) ethoxy)prop-1-yn-1-yl)thiazol-5-yl)phenyl)ethyl)pyrrolidine-2-carboxamide

To a solution of (2S,4R)-4-((tert-butyldimethylsilyl)oxy)-N—((S)-1-(4-(4-(3-(2-(2-hydroxyethoxy) ethoxy)prop-1-yn-1-yl)thiazol-5-yl)phenyl)ethyl)-1-((R)-3-methyl-2-(3-methylisoxazol-5-yl)butanoyl)pyrrolidine-2-carboxamide (24.0 mg, 1 eq, 32.5 μmol) in DCM (4 mL) was added DMP (55.1 mg, 4 eq, 130 μmol). The mixture was stirred at 25° C. for 4 hours. LCMS indicated completion of reaction. The mixture was poured into water (20 mL), extracted with EA (20 mL×3). The combined organic layers were washed with brine (30 mL), dried over sodium sulfate, filtered and concentrated under vacuum. The resulting residue was purified by silica gel column chromatography (100-200 mesh silica gel, 6% of MeOH in DCM) to afford (2S,4R)-4-((tert-butyldimethylsilyl)oxy)-1-((R)-3-methyl-2-(3-methylisoxazol-5-yl)butanoyl)-N—((S)-1-(4-(4-(3-(2-(2-oxoethoxy) ethoxy)prop-1-yn-1-yl)thiazol-5-yl)phenyl)ethyl)pyrrolidine-2-carboxamide (15.0 mg, 14 μmol, 44%, 70% purity) as a yellow solid. LC purity (0.1% FA): 64.48% (UV at 254 nm)/MS: 737.1 [M+1]; Retention time: 1.62 min.

### Synthesis of (2S,4R)-4-((tert-butyldimethylsilyl)oxy)-N—((S)-1-(4-(4-(3-(2-(2-(4-(4-chloro-7,7-dimethyl-5-oxo-5,7-dihydroindolo[1,2-a]quinazolin-10-yl)piperidin-1-yl)ethoxy) ethoxy)prop-1-yn-1-yl)thiazol-5-yl)phenyl)ethyl)-1-((R)-3-methyl-2-(3-methylisoxazol-5-yl)butanoyl)pyrrolidine-2-carboxamide

To a solution of (2S,4R)-4-hydroxy-1-((R)-3-methyl-2-(3-methylisoxazol-5-yl)butanoyl)-N—((S)-1-(4-(4-(3-(2-(2-oxoethoxy) ethoxy)prop-1-yn-1-yl)thiazol-5-yl)phenyl)ethyl)pyrrolidine-2-carboxamide (20.0 mg, 70% Wt, 1 eq, 22.5 μmol) in MeOH (1 mL) were added 4-chloro-7,7-dimethyl-10-(piperidin-4-yl)indolo[1,2-a]quinazolin-5 (7H)-one (8.54 mg, 1 eq, 22.5 μmol), TEA (6.82 mg, 9.40 μL, 3 eq, 67.4 μmol) and NaBH₃CN (5.65 mg, 4 eq, 89.9 μmol). The mixture was stirred at 25° C. for 4 hours. LCMS indicated completion of reaction. The mixture was poured into water (20 mL), extracted with DCM (20 mL×3). The combined organic layers were washed with brine (30 mL), dried over sodium sulfate, filtered and concentrated under vacuum. The resulting residue was purified by column (100-200 mesh silica gel, 5% of MeOH in DCM) to afford (2S,4R)-4-((tert-butyldimethylsilyl)oxy)-N—((S)-1-(4-(4-(3-(2-(2-(4-(4-chloro-7,7-dimethyl-5-oxo-5,7-dihydroindolo[1,2-a]quinazolin-10-yl)piperidin-1-yl)ethoxy) ethoxy)prop-1-yn-1-yl)thiazol-5-yl)phenyl)ethyl)-1-((R)-3-methyl-2-(3-methylisoxazol-5-yl)butanoyl)pyrrolidine-2-carboxamide (12.0 mg, 8.7 μmol, 39%, 80% purity) as a yellow solid. LC purity (0.1% FA): 54.42% (UV at 254 nm)/MS: 551.2 [M/2+H]; Retention time: 1.52 min.

### Synthesis of (2S,4R)—N—((S)-1-(4-(4-(3-(2-(2-(4-(4-chloro-7,7-dimethyl-5-oxo-5,7-dihydroindolo[1,2-a]quinazolin-10-yl)piperidin-1-yl)ethoxy) ethoxy)prop-1-yn-1-yl)thiazol-5-yl)phenyl)ethyl)-4-hydroxy-1-(3-methyl-2-(3-methylisoxazol-5-yl)butanoyl)pyrrolidine-2-carboxamide

To a solution of (2S,4R)-4-((tert-butyldimethylsilyl)oxy)-N—((S)-1-(4-(4-(3-(2-(2-(4-(4-chloro-7,7-dimethyl-5-oxo-5,7-dihydroindolo[1,2-a]quinazolin-10-yl)piperidin-1-yl)ethoxy) ethoxy)prop-1-yn-1-yl)thiazol-5-yl)phenyl)ethyl)-1-(3-methyl-2-(3-methylisoxazol-5-yl)butanoyl)pyrrolidine-2-carboxamide (12.0 mg, 1 eq, 10.9 μmol) in DCM (2 mL) was added TFA (2 mL). The mixture was stirred at 35° C. for 16 hours. LCMS indicated completion of reaction. The reaction was filtered and concentrated under vacuum. The resulting residue was purified by prep-HPLC (Waters 3767/Qda Column: Sunfire C18, 19*250 mm, 10 um; Mobile Phase A: 10 mmol NH₄HCO₃/H₂O, B:ACN; flow rate: 20 ml/min; gradient: 45-55% Retention Time: 7.6 of 16 min) to afford (2S,4R)—N—((S)-1-(4-(4-(3-(2-(2-(4-(4-chloro-7,7-dimethyl-5-oxo-5,7-dihydroindolo[1,2-a]quinazolin-10-yl)piperidin-1-yl)ethoxy) ethoxy)prop-1-yn-1-yl)thiazol-5-yl)phenyl)ethyl)-4-hydroxy-1-(3-methyl-2-(3-methylisoxazol-5-yl)butanoyl)pyrrolidine-2-carboxamide (2.12 mg, 1.95 μmol, 17.9%, 90.57% purity) as a yellow solid. LC purity (0.1% FA): 66.02% (UV at 254 nm)/MS: 493.5 [½M+1]; Retention time: 1.39 min. ¹H NMR (400 MHz, MeOD-d₄) δ 8.68-8.77 (m, 1H), 8.37 (d, J=8.4 Hz, 1H), 7.90 (d, J=5.4 Hz, 1H), 7.78-7.82 (m, 1H), 7.63-7.71 (m, 3H), 7.57 (d, J=8.1 Hz, 1H), 7.38 (d, J=8.2 Hz, 1H), 7.26-7.31 (m, 2H), 6.23 (d, J=12 Hz, 1H), 5.34-5.36 (m, 1H), 4.51 (s, 2H), 3.86-3.89 (m, 2H), 3.75-3.80 (m, 6H), 3.40-3.42 (m, 2H), 3.19-3.21 (m, 2H), 2.41-2.45 (m, 1H), 2.24 (d, J=4.0 Hz, 2H), 2.20 (d, J=9.2 Hz, 2H), 2.10-2.14 (m, 4H), 1.93 (s, 1H), 1.59 (s, 6H), 1.49 (d, J=6.9 Hz, 1H), 1.44 (d, J=6.9 Hz, 1H), 1.29-1.33 (m, 6H), 1.05 (dd, J=6.5, 3.6 Hz, 3H), 0.84-0.90 (m, 4H).

The examples in the table below were prepared according to the same method using appropriate starting materials.

| | Calcd. | Found | |
|---|---|---|---|
| | (M + H)⁺ | (M + H)⁺ | |
| Ex. | m/z | m/z | NMR |
| B103 | 956.1 | 956.1 | ¹H NMR (400 MHz, MeOD-d₄) δ 9.28 (s, |
| | | | 1H), 8.42 (d, J = 8.3 Hz, 1H), 8.27-8.10 (m, |
| | | | 3H), 7.93 (s, 1H), 7.85 (t, J = 8.4 Hz, 1H), |
| | | | 7.69-7.64 (m, 2H), 7.55 (d, J = 7.8 Hz, 1H), |
| | | | 7.29 (d, J = 7.9 Hz, 1H), 6.21 (s, 1H), 5.16- |
| | | | 5.05 (m, 2H), 4.64-4.53 (m, 2H), 4.45-4.43 |
| | | | (m, 1H), 3.88-3.53 (m, 6H), 3.05 (d, J = 10.7 |
| | | | Hz, 2H), 2.76-2.74 (m, 1H), 2.55-2.53 (m, |
| | | | 2H), 2.43-2.40 (m, 1H), 2.27-2.11 (m, 6H), |
| | | | 1.87-1.85 (m, 6H), 1.67-1.54 (m, 9H), 1.06 (t, |
| | | | J = 8.2 Hz, 3H), 0.86 (dd, J = 11.3, 6.8 Hz, |
| | | | 3H). |

### Compound B105. (2S,4R)-1-((S)-2-(4-((1r,4S)-4-((4-(4-bromo-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-hydroxy-N—((R)-1-(4-(4-methylthiazol-5-yl)phenyl)-2-carboxamide

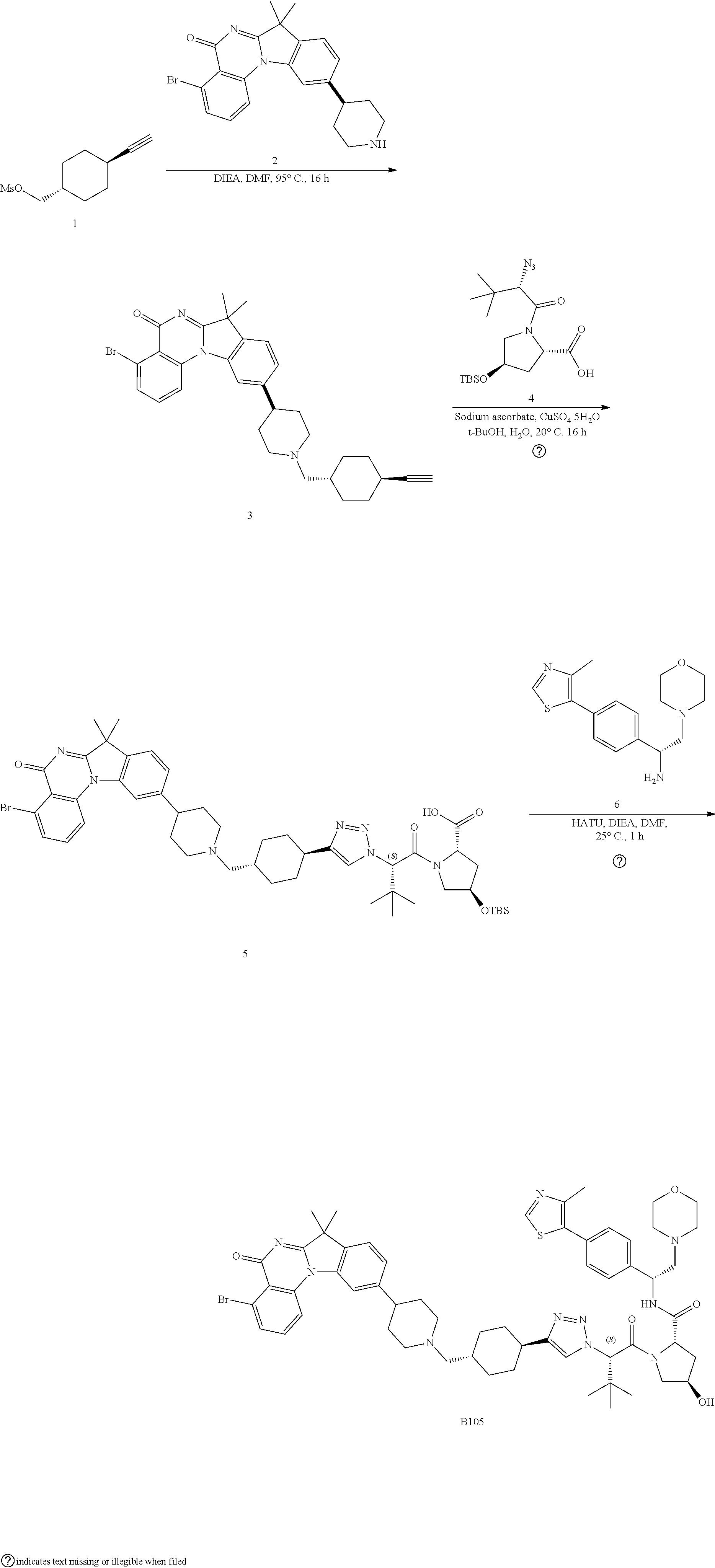

### Synthesis of tert-butyl 4-bromo-10-(1-(((1r,4r)-4-ethynylcyclohexyl)methyl)piperidin-4-yl)-7,7-dimethylindolo[1,2-a]quinazolin-5 (7H)-one (Intermediate 3)

To a mixture of 4-bromo-7,7-dimethyl-10-(piperidin-4-yl)indolo[1,2-a]quinazolin-5 (7H)-one (500 mg, 1 eq, 1.18 mmol) and ((1r,4r)-4-ethynylcyclohexyl)methyl methanesulfonate (458.8 mg, 1.8 eq, 2.12 mmol) in DMF (10 mL) was added DIEA (0.62 mL, 3 eq). The reaction mixture was stirred at 95° C. for 16 h under N₂. LCMS indicated completion of reaction. The reaction mixture was diluted with water, extracted with ethyl acetate (30.0 mL) and concentrated under vacuum. The residue was purified by silica gel column chromatography (100-200 mesh silica gel, 0-10% of methanol in dichloromethane) to afford 4-bromo-10-(1-(((1r,4r)-4-ethynylcyclohexyl)methyl)piperidin-4-yl)-7,7-dimethylindolo[1,2-a]quinazolin-5 (7H)-one (350 mg, 0.64 mmol, 54.5%) as a yellow solid. LC purity (0.03% TFA): 66.24% (UV at 2154 nm)/MS: 544.2 [M+H]; Retention time: 1.34 min.

### Synthesis of (2S,4R)-1-((S)-2-(4-((1r,4S)-4-((4-(4-bromo-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-((tert-butyldimethylsilyl)oxy)-N—((R)-1-(4-(4-methylthiazol-5-yl)phenyl)-2-morpholinoethyl)pyrrolidine-2-carboxamide (Intermediate 5)

To a mixture of 4-bromo-10-(1-(((1r,4r)-4-ethynylcyclohexyl)methyl)piperidin-4-yl)-7,7-dimethylindolo[1,2-a]quinazolin-5 (7H)-one (200 mg, 1 eq, 0.37 mmol) and (2S,4R)-1-((S)-2-azido-3,3-dimethylbutanoyl)-4-((tert-butyldimethylsilyl)oxy)pyrrolidine-2-carboxylic acid (254.2 mg, 1.8 eq, 0.66 mmol) in t-BuOH (4 mL) and Water (4 mL) was added sodium (R)-2-((S)-1,2-dihydroxyethyl)-4-hydroxy-5-oxo-2,5-dihydrofuran-3-olate (72.76 mg, 1 eq, 0.37 mmol) and copper (II) sulfatepentahydrate (73.36 mg, 0.8 eq, 0.3 mmol), the mixture was stirred at 25° C. for 24 h. LCMS indicated completion of reaction. The reaction mixture was extracted with ethyl acetate (30 mL) and concentrated under vacuum. The residue was purified by silica gel prep-TLC (DCM/MeOH=15/1) to afford (2S,4R)-1-((S)-2-(4-((1r,4S)-4-((4-(4-bromo-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-((tert-butyldimethylsilyl)oxy)pyrrolidine-2-carboxylic acid (175 mg, 0.19 mmol, 51.3%) as a yellow solid. LC purity (0.1% TFA): 97.87% (UV at 254 nm)/MS: 928.4 [M+H]; Retention time: 1.58 min.

### Synthesis of tert-butyl (2S,4R)-1-((S)-2-(4-((1r,4S)-4-((4-(4-bromo-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-hydroxy-N—((R)-1-(4-(4-methylthiazol-5-yl)phenyl)-2-morpholinoethyl)pyrrolidine-2-carboxamide (B105)

To a mixture of (2S,4R)-1-((S)-2-(4-((1r,4S)-4-((4-(4-bromo-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-((tert-butyldimethylsilyl)oxy)pyrrolidine-2-carboxylic acid (80 mg, 1 eq, 0.86 mmol) and (R)-1-(4-(4-methylthiazol-5-yl)phenyl)-2-morpholinoethan-1-amine (39.2 mg, 1.5 eq, 0.129 mmol) in DMF (2 mL) was added DIEA (33.4 mg, 3 eq, 0.26 mmol) and BOP (57.1 mg, 1.5 eq, 0.13 mmol). The mixture was stirred at 25° C. for 1 h under N₂. LCMS indicated completion of reaction. The reaction mixture was filtered. The crude product was purified by silica prep-HPLC (0.01 NH₄HCO₃%, eluted with H₂O and CH₃CN from 90/10 to 5/95) to afford (2S,4R)-1-((S)-2-(4-((1r,4S)-4-((4-(4-bromo-7,7-dimethyl-5-oxo-5,7-dihydroindolo[1,2-a]quinazolin-10-yl)piperidin-1-yl)methyl)cyclohexyl)-1H-1,2,3-triazol-1-yl)-3,3-dimethylbutanoyl)-4-hydroxy-N—((R)-1-(4-(4-methylthiazol-5-yl)phenyl)-2-morpholinoethyl)pyrrolidine-2-carboxamide (14.73 mg, 12.49 μmol, 14.5%) as a white solid. LC purity (0.1% NH₄HCO₃): 93.05% (UV at 254 nm)/MS: 550.4 [M/2+H]; Retention time: 1.32 min. ¹H NMR (400 MHz, MeOD) δ 8.87-8.85 (m, 1H), 8.49 (d, J=8.4 Hz, 1H), 8.02-8.01 (m, 1H), 7.97 (s, 1H), 7.90 (d, J=7.9 Hz, 1H), 7.77 (t, J=8.4 Hz, 1H), 7.56 (d, J=7.6 Hz, 1H), 7.49-7.39 (m, 4H), 7.34 (t, J=6.8 Hz, 1H), 5.48 (s, 1H), 5.13-4.96 (m, 1H), 4.60-4.56 (m, 6H), 4.45-4.43 (m, 1H), 3.87-3.83 (m, 1H), 3.79-3.74 (m, 4H), 3.14-3.11 (m, 2H), 2.84-2.73 (m, 3H), 2.69-2.52 (m, 5H), 2.48 (s, 3H), 2.32-2.30 (m, 2H), 2.24-2.08 (m, 5H), 2.00-1.90 (m, 6H), 1.59 (s, 6H), 1.51-1.48 (m, 2H), 1.07 (d, J=5.6 Hz, 9H).

The examples in the table below were prepared according to the same method as example B105 using appropriate starting materials

| | Calcd. | Found | |
|---|---|---|---|
| | (M + H)+ | (M + H)+ | |
| Ex. | m/z | m/z | HNMR |
| B106 | 1125.5 | 563.4 | 1H NMR (400 MHz, MeOD) δ 8.90 (s, 1H), |
| | | | 8.45 (d, J = 8.6 Hz, 1H), 7.98 (s, 1H), 7.93- |
| | | | 7.84 (m, 3H), 7.74 (t, J = 8.2 Hz, 1H), 7.55- |
| | | | 7.46 (m, 4H), 7.34 (d, J = 7.6 Hz, 1H), 5.51 |
| | | | (s, 1H), 5.30 (d, J = 10.4 Hz, 1H), 4.52-4.50 |
| | | | (m, 2H), 4.01-3.85 (m, 6H), 3.80-3.78 (m, |
| | | | 2H), 3.68-3.50 (m, 2H), 3.37-3.36 (m, 2H), |
| | | | 3.23-3.09 (m, 7H), 2.80-2.78 (m, 1H), 2.61- |
| | | | 2.60 (m, 1H), 2.48 (s, 3H), 2.27-2.10 (m, |
| | | | 9H), 2.02-2.00 (m, 6H), 1.85-1.82 (m, 3H), |
| | | | 1.78-1.76 (m, 3H), 1.63-1.52 (m, 2H), 1.27- |
| | | | 1.37 (m, 2H), 1.14 (d, J = 6.5 Hz, 3H), 0.79 |
| | | | (d, J = 6.5 Hz, 3H). |
| B107 | 956.4 | 478.8 | 1H NMR (400 MHz, MeOD) δ 8.88 (s, 1H), |
| | | | 8.44 (d, J = 8.4 Hz, 1H), 8.15-8.00 (m, 1H), |
| | | | 7.86-7.84 (m, 2H), 7.70-7.54 (m, 2H), 7.48- |
| | | | 7.30 (m, 5H), 5.28 (d, J = 10.3 Hz, 1H), 5.08- |
| | | | 5.06 (m, 1H), 4.56-4.40 (m, 2H), 3.99-3.71 |
| | | | (m, 4H), 3.60-3.40 (m, 2H), 3.27-3.25 (m, |
| | | | 1H), 3.12-3.11 (m, 1H), 2.78-2.75 (m, 1H), |
| | | | 2.56-2.55 (m, 1H), 2.48 (s, 3H), 2.34-2.10 (m, |
| | | | 6H), 2.08-1.94 (m, 4H), 1.77-1.76 (m, 5H), |
| | | | 1.71-1.55 (m, 4H), 1.53 (d, J = 7.0 Hz, 3H), |
| | | | 1.35-1.33 (m, 3H), 1.15 (d, J = 6.0 Hz, 3H), |
| | | | 0.74 (d, J = 6.2 Hz, 3H). |
| B108 | 1123.6 | 1123.2 | 1H NMR (400 MHz, MeOD) δ 8.94 (s, 1H), |
| | | | 8.42-8.40 (d, J = 8.8 Hz, 1H), 8.05-8.02 (d, |
| | | | J = 5.6 Hz, 1H), 8.00 (s, 1H), 7.88-7.85 (m, |
| | | | 2H), 7.66 (d, J = 8.0 Hz, 1H), 7.43 (s, 3H), |
| | | | 7.43-7.38 (m, 2H), 5.52-5.50 (m, 1H), 5.04- |
| | | | 4.85 (m, 1H), 4.60-4.50 (m, 1H), 4.45-4.44 |
| | | | (m, 1H), 3.95-3.78 (m, 1H), 3.77-3.74 (m, |
| | | | 7H), 3.16-3.10 (m, 5H), 2.97-2.66 (m, 9H), |
| | | | 2.21-2.10 (m, 8H), 2.08-2.01 (m, 7H), 1.98- |
| | | | 1.83 (m, 7H), 1.68-1.58 (m, 3H), 1.55-1.52 |
| | | | (d, J = 7.2 Hz, 3H), 1.32-1.28 (m, 2H), 1.08 |
| | | | (s, 9H). |
| B112 | 1139.5 | 570.4 | 1H NMR (400 MHz, MeOD) δ 8.87 (s, 1H), |
| | | | 8.46 (d, J = 8.4 Hz, 1H), 8.04 (d, J = 6.4 Hz, |
| | | | 1H), 7.98 (s, 1H), 7.90-7.88 (m, 2H), 7.78- |
| | | | 7.76 (m, 1H), 7.46-7.44 (m, 4H), 7.39-7.29 |
| | | | (m, 1H), 5.50 (s, 1H), 5.18-5.04 (m, 1H), 4.58 |
| | | | (t, J = 8.4 Hz, 1H), 4.45 (s, 1H), 3.86 (d, J = |
| | | | 10.4 Hz, 1H), 3.75 (d, J = 4.4 Hz, 6H), 3.53- |
| | | | 3.50 (m, 3H), 2.93-2.72 (m, 6H), 2.72-2.60 |
| | | | (m, 3H), 2.50-2.47 (m, 3H), 2.16-2.12 (m, |
| | | | 8H), 2.10-1.99 (m, 4H), 1.93-1.89 (m, 45H), |
| | | | 1.79-1.66 (m, 3H), 1.59-1.56 (m, 3H), 1.28- |
| | | | 1.26 (m, 3H), 1.06 (s, 9H). |
| B113 | 997.5 | 499.4 | 1H NMR (400 MHz, MeOD) δ 8.90 (s, 1H), |
| | | | 8.45 (d, J = 8.8 Hz, 1H), 8.04 (t, J = 16.4 Hz, |
| | | | 2H), 7.87 (t, J = 8.0 Hz, 1H), 7.76-7.44 (m, |
| | | | 6H), 7.37 (d, J = 7.6 Hz, 1H), 5.50 (s, 1H), |
| | | | 4.56-4.53 (m, 5H), 3.85-3.83 (m, 3H), 3.67- |
| | | | 3.66 (m, 2H), 3.48-3.46 (m, 1H), 3.19-2.95 |
| | | | (m, 6H), 2.79-2.77 (m, 1H), 2.50 (s, 3H), |
| | | | 2.23-2.21 (m, 7H), 2.05-2.03 (m, 3H), 1.61- |
| | | | 1.60 (m, 7H), 1.28-1.26 (m, 3H), 1.07-1.05 |
| | | | (m, 9H). |
| B117 | 991.4 | 991.4 | 1H NMR (400 MHz, MeOD) δ 8.94 (s, 1H), |
| | | | 8.43 (d, J = 8.7 Hz, 1H), 8.06-8.04 (m, 2H), |
| | | | 7.87 (t, J = 8.3 Hz, 1H), 7.70-7.58 (m, 4H), |
| | | | 7.47-7.34 (m, 3H), 5.51 (s, 1H), 5.04 (d, J = |
| | | | 7.1 Hz, 1H), 4.57-4.55 (m, 4H), 4.43-4.41 (m, |
| | | | 1H), 3.88-3.86 (m, 1H), 3.75-3.74 (m, 1H), |
| | | | 3.66-3.63 (m, 2H), 3.59-3.45 (m, 1H), 3.02- |
| | | | 2.98 (m, 5H), 2.79-2.77 (m, 1H), 2.18-2.16 |
| | | | (m, 6H), 2.01-1.97 (m, 4H), 1.60 (s, 6H), 1.54 |
| | | | (d, J = 7.0 Hz, 3H), 1.29 (d, J = 11.4 Hz, 2H), |
| | | | 1.08 (s, 9H). |
| B118 | 1145.5 | 1145.7 | 1H NMR (400 MHz, MeOD) δ 8.94-8.87 (m, |
| | | | 1H), 8.45 (d, J = 8.8 Hz, 1H), 8.10-8.08 (m, |
| | | | 1H), 7.97 (s, 1H), 7.90 (d, J = 8.0 Hz, 2H), |
| | | | 7.68-7.65 (m, 1H), 7.41-7.35 (m, 3H), 7.32- |
| | | | 7.24 (m, 2H), 5.56-5.55 (m, 1H), 5.42-5.34 |
| | | | (m, 1H), 5.21-5.13 (m, 1H), 4.58-4.55 (m, |
| | | | 4H), 4.03-3.98 (m, 2H), 3.85-m), 3.21-3.15 |
| | | | (m, 2H), 2.98-2.96 (m, 1H), 2.30-2.78 (m, |
| | | | 2H), 2.71-2.61 (m, 4H), 2.53-2.48 (m, 3H), |
| | | | 2.33-2.30 (m, 2H), 2.28-2.25 (m, 2H), 2.04- |
| | | | 1.98 (m, 4H), 1.92-1.86 (m, 4H), 1.80-1.75 |
| | | | (m, 4H), 1.62-1.60 (m, 2H), 1.35-1.33 (m, |
| | | | 4H), 1.07 (s, 9H). |
| B119 | 964.4 | 964.6 | 1H NMR (400 MHz, MeOD) δ 8.85 (s, 1H), |
| | | | 8.63 (s, 1H), 8.49-8.43 (m, 1H), 7.98 (s, 1H), |
| | | | 7.94-7.96 (m, 1H), 7.86-7.92 (m, 1H), 7.78- |
| | | | 7.82 (m, 1H), 7.69-7.65 (m, 1H), 7.62-7.55 |
| | | | (m, 1H), 7.52-7.45 (m, 4H), 7.41-7.36 (m, |
| | | | 2H), 7.38-7.33 (m, 1H), 5.61 (s, 1H), 5.12- |
| | | | 5.05 (m, 1H), 4.62-4.58 (m, 2H), 4.48-4.47 |
| | | | (m, 1H), 3.95-3.89 (m, 1H), 3.86-3.81 (m, |
| | | | 1H), 3.71-3.70 (m, 2H), 3.24-3.14 (m, 2H), |
| | | | 2.82-2.80 (m, 1H), 2.50 (s, 3H), 2.34-2.30 |
| | | | (sm, 2H), 1.98-1.94 (m, 4H), 1.58 (s, 6H), |
| | | | 1.58-1.53 (m, 3H), 1.30-1.28 (m, 2H), 1.16 (s, |
| | | | 9H). |
| B120 | 957.4 | 957.5 | 1H NMR (400 MHz, MeOD) δ 8.87 (s, 1H), |
| | | | 8.39 (d, J = 8.8 Hz, 1H), 7.93-7.81 (m, 2H), |
| | | | 7.68-7.57 (m, 2H), 7.48-7.41 (m, 4H), 7.02 |
| | | | (d, J = 10.1 Hz, 1H), 5.26 (d, J = 10.4 Hz, |
| | | | 1H), 5.04 (d, J = 7.2 Hz, 1H), 4.52-4.50 (m, |
| | | | 2H), 3.95-3.76 (m, 2H), 3.36-3.32 (m, 8H), |
| | | | 2.69-2.67 (m, 4H), 2.48 (s, 3H), 2.32 (d, J = |
| | | | 7.2 Hz, 2H), 2.21-2.16 (m, 1H), 2.10 (d, J = |
| | | | 12.8 Hz, 2H), 1.98-1.96 (m, 3H), 1.74-1.63 |
| | | | (m, 2H), 1.57 (s, 6H), 1.56-1.54 (m, 1H), |
| | | | 1.53-1.52 (m, 2H), 1.50-1.49 (m, 1H), 1.13 |
| | | | (d, J = 6.4 Hz, 3H), 0.95 (t, J = 7.2 Hz, 1H), |
| | | | 0.74 (d, J = 6.2 Hz, 3H). |
| B121 | 971.4 | 971.6 | 1H NMR (400 MHz, MeOD) δ 8.87 (s, 1H), |
| | | | 8.39 (d, J = 8.8 Hz, 1H), 8.01 (s, 1H), 7.86 (t, |
| | | | J = 8.4 Hz, 1H), 7.68-7.57 (m, 2H), 7.48-7.45 |
| | | | (m, 4H), 7.02 (d, J = 8.4 Hz, 1H), 5.50 (s, |
| | | | 1H), 5.13-5.09 (m, 1H), 4.65-4.51 (m, 1H), |
| | | | 4.46-4.45 (m, 1H), 3.92-3.90 (m, 1H), 3.75 |
| | | | (d, J = 11.2 Hz, 1H), 3.37-3.32 (m, 6H), |
| | | | 2.69-2.67 (m, 4H), 2.48 (s, 3H), 2.31 (d, J = |
| | | | 6.8 Hz, 2H), 2.25-2.18 (m, 1H), 2.10 (d, J = |
| | | | 10.8 Hz, 2H), 2.01-1.98 (m, 3H), 1.73-1.70 |
| | | | (m, 2H), 1.57 (s, 6H), 1.56-1.53 (m, 1H), |
| | | | 1.52-1.51 (m, 2H), 1.17-1.15 (m, 2H), 1.07 (s, |
| | | | 9H), 0.95 (t, J = 7.2 Hz, 1H). |
| B122 | 982.4 | 982.4 | 1H NMR (400 MHz, MeOD) δ 8.89-8.86 (m, |
| | | | 1H), 8.44 (d, J = 8.6 Hz, 1H), 8.08-8.02 (m, |
| | | | 1H), 7.97 (s, 1H), 7.90-7.86 (m, 1H), 7.66 (d, |
| | | | J = 7.8 Hz, 1H), 7.60-7.56 (m, 1H), 7.44-7.26 |
| | | | (m, 4H), 5.52 (d, J = 4.7 Hz, 1H), 5.48-5.38 |
| | | | (m, 1H), 4.60-4.58 (m, 4H), 4.51-4.46 (m, |
| | | | 2H), 3.99-3.89 (m, 1H), 3.83-3.71 (m, 1H), |
| | | | 3.21-3.05 (m, 4H), 2.85-2.71 (m, 2H), 2.48 |
| | | | (d, J = 4.1 Hz, 3H), 2.41-2.38 (m, 1H), 2.26- |
| | | | 2.20 (m, 1H), 2.16-2.09 (m, 3H), 2.00-1.92 |
| | | | (m, 6H), 1.79-1.68 (m, 1H), 1.59 (s, 6H), |
| | | | 1.54-1.52 (m, 2H), 1.36-1.25 (s, 3H), 1.15- |
| | | | 1.05 (m, 9H). |
| B123 | 982.4 | 982.4 | 1H NMR (400 MHz, MeOD) δ 8.88 (s, 1H), |
| | | | 8.43 (d, J = 8.6 Hz, 1H), 8.10-8.08 (m, 1H), |
| | | | 8.00 (s, 1H), 7.86 (t, J = 8.1 Hz, 1H), 7.70- |
| | | | 7.57 (m, 3H), 7.38-7.29 (m, 3H), 5.54 (d, J = |
| | | | 5.1 Hz, 1H), 5.43-5.41 (m, 1H), 4.55-4.48 (m, |
| | | | 2H), 3.94 (d, J = 10.3 Hz, 1H), 3.82-3.79 (m, |
| | | | 3H), 3.16-3.12 (m, 6H), 2.98-2.96 (m, 1H), |
| | | | 2.79-2.78 (m, 1H), 2.48 (d, J = 4.3 Hz, 3H), |
| | | | 2.24-2.18 (m, 8H), 2.01-1.90 (m, 3H), 1.61 (s, |
| | | | 9H), 1.35-1.31 (m, 2H), 1.16-1.08 (m, 10H). |
| B124 | 1011.5 | 1011.1 | 1H NMR (400 MHz, MeOD) δ 8.91 (s, 1H), |
| | | | 8.42 (d, J = 8.4 Hz, 1H), 8.11-7.94 (m, 2H), |
| | | | 7.86 (t, J = 8.3 Hz, 1H), 7.77-7.38 (m, 7H), |
| | | | 5.50 (s, 1H), 4.69-4.47 (m, 3H), 4.36-4.35 (m, |
| | | | 3H), 3.90-3.84 (m, 1H), 3.80-3.61 (m, 3H), |
| | | | 3.5-3.48 (m, 1H), 3.19-3.13 (m, 5H), 2.92- |
| | | | 2.63 (m, 5H), 2.50 (s, 3H), 2.31-1.97 (m, |
| | | | 11H), 1.61 (s, 6H), 1.33-1.30 (m, 2H), 1.07- |
| | | | 1.00 (m, 9H). |
| B125 | 954.5 | 954.5 | 1H NMR (400 MHz, MeOD) δ 8.40 (d, J = |
| | | | 8.8 Hz, 1H), 8.02 (s, 1H), 7.85 (t, J = 8.4 Hz, |
| | | | 1H), 7.63-7.59 (m, 2H), 7.52-7.40 (m, 6H), |
| | | | 7.01 (dd, J = 8.4, 1.6 Hz, 1H), 6.34 (t, J = 2.4 |
| | | | Hz, 1H), 5.50 (s, 1H), 5.08-5.01 (m, 1H), |
| | | | 4.58-4.52 (m, 1H), 4.46-4.45 (m, 1H), 3.90- |
| | | | 3.81 (m, 4H), 3.79-3.73 (m, 1H), 3.39-3.32 |
| | | | (m, 4H), 2.75-2.72 (m, 1H), 2.68-2.59 (m, |
| | | | 4H), 2.30 (d, J = 7.2 Hz, 2H), 2.23-2.19 (m, |
| | | | 1H), 2.10-2.07 (m, 2H), 2.03-1.98 (m, 2H), |
| | | | 1.74-1.63 (m, 2H), 1.56 (s, 6H), 1.53 (d, J = |
| | | | 7.2 Hz, 3H), 1.49-1.46 (m, 2H), 1.20-1.11 (m, |
| | | | 2H), 1.07 (s, 9H). |
| B126 | 955.5 | 955.8 | 1H NMR (400 MHz, MeOD) δ 8.40 (d, J = |
| | | | 8.7 Hz, 1H), 8.14 (s, 1H), 8.04 (d, J = 7.7 Hz, |
| | | | 1H), 7.85 (t, J = 8.2 Hz, 1H), 7.71-7.58 (m, |
| | | | 4H), 7.52 (d, J = 8.2 Hz, 1H), 7.43-7.41 (m, |
| | | | 2H), 7.05 (d, J = 8.6 Hz, 1H), 5.51 (s, 1H), |
| | | | 5.12-4.94 (m, 1H), 4.65-4.39 (m, 3H), 3.88- |
| | | | 3.69 (m, 2H), 3.55-3.52 (m, 4H), 3.23-3.21 |
| | | | (m, 3H), 2.80-2.78 (m, 3H), 2.40 (s, 3H), |
| | | | 2.31-2.09 (m, 3H), 2.05-1.82 (m, 4H), 1.64 |
| | | | (d, J = 6.9 Hz, 1H), 1.57 (s, 6H), 1.55-1.53 |
| | | | (m, 4H), 1.27-1.25 (m, 2H), 1.07 (s, 9H). |
| B127 | 940.5 | 940.8 | 1H NMR (400 MHz, DMSO) δ 8.55 (d, J = |
| | | | 7.6 Hz, 1H), 8.35 (d, J = 8.4 Hz, 1H), 8.04- |
| | | | 7.98 (m, 1H), 7.83-7.78 (m, 1H), 7.76-7.68 |
| | | | (m, 3H), 7.65-7.59 (m, 2H), 7.52 (d, J = 8.4 |
| | | | Hz, 1H), 7.34-7.28 (m, 2H), 7.07-7.01 (m, |
| | | | 1H), 6.65 (s, 1H), 5.51 (s, 1H), 5.07-4.94 (m, |
| | | | 1H), 4.53-4.48 (m, 1H), 4.42-4.38 (m, 1H), |
| | | | 4.01-3.98 (m, 1H), 3.85-3.78 (m, 1H), 3.78- |
| | | | 3.68 (m, 2H), 3.36-3.34 (m, 2H), 3.18 (d, J = |
| | | | 6.4 Hz, 2H), 2.78-2.74 (m, 1H), 2.18-2.10 (m, |
| | | | 3H), 2.05-1.95 (m, 4H), 1.63-1.58 (m, 2H), |
| | | | 1.57 (s, 6H), 1.52-1.48 (m, 2H), 1.30-1.24 (m, |
| | | | 5H), 1.07 (s, 9H). |
| B128 | 883.4 | 442.4 | 1H NMR (400 MHz, MeOD) δ 8.42 (d, J = |
| | | | 8.4 Hz, 1H), 8.00 (s, 1H), 7.94-7.82 (m, 2H), |
| | | | 77.68-.65 (m, 2H), 7.42-7.40 (m, 2H), 7.33- |
| | | | 7.23 (m, 2H), 5.27 (d, J = 10.4 Hz, 1H), 5.06- |
| | | | 4.93 (m, 1H), 4.56-4.44 (m, 2H), 3.95-3.68 |
| | | | (m, 4H), 3.44 (s, 1H), 3.18-3.12 (m, 5H), |
| | | | 2.79-2.77 (m, 1H), 2.57-2.55 (m, 1H), 2.23- |
| | | | 2.19 (m, 7H), 2.04-1.89 (m, 4H), 1.60 (s, 6H), |
| | | | 1.56-1.55 (m, 1H), 1.48 (d, J = 7.2 Hz, 3H), |
| | | | 1.40-1.26 (m, 3H), 1.13-1.11 (m, 3H), 0.74 |
| | | | (d, J = 7.2 Hz, 3H). |
| B129 | 1042.5 | 1042.8 | 1H NMR (400 MHz, MeOD) δ 8.91 (s, 1H), |
| | | | 8.40 (d, J = 8.0 Hz, 1H), 7.90 (s, 1H), 7.85 (t, |
| | | | J = 8.4 Hz, 1H), 7.73-7.65 (m, 2H), 7.55-7.49 |
| | | | (m, 4H), 7.08 (d, J = 8.0 Hz, 1H), 5.32 (d, J = |
| | | | 10.4 Hz, 1H), 4.95-4.93 (m, 4H), 4.52-4.50 |
| | | | (m, 1H), 4.00-3.94 (m, 8H), 3.50-3.48 (m, |
| | | | 4H), 3.23-3.25 (m, 8H), 3.29-3.17 (m, 2H), |
| | | | 3.15-3.13 (m, 1H), 2.81-2.79 (m, 1H), 2.65- |
| | | | 2.58 (m, 1H), 2.48 (s, 3H), 2.15-2.13 (m, 3H), |
| | | | 2.04-2.02 (m, 4H), 1.58 (s, 6H), 1.35-1.33 (m, |
| | | | 2H), 1.16 (d, J = 6.4 Hz, 3H), 0.80 (d, J = 6.8 |
| | | | Hz, 3H). |
| B131 | 897.5 | 897.56 | 1H NMR (400 MHz, MeOD) δ 8.43 (d, J = |
| | | | 8.6 Hz, 1H), 8.00 (s, 1H), 7.92-7.83 (m, 2H), |
| | | | 7.65 (dd, J = 18.0, 7.9 Hz, 2H), 7.37 (d, J = |
| | | | 7.8 Hz, 1H), 7.30 (d, J = 7.8 Hz, 2H), 7.25- |
| | | | 7.18 (m, 2H), 5.27 (d, J = 10.2 Hz, 1H), 4.96 |
| | | | (s, 1H), 4.60-4.58 (m, 2H), 4.52-4.38 (m, 2H), |
| | | | 4.10 (q, J = 7.1 Hz, 1H), 3.91-3.89 (m, 1H), |
| | | | 3.82 (d, J = 10.9 Hz, 1H), 3.74-3.72 (m, 2H), |
| | | | 3.18-3.11 (m, 4H), 2.79-2.77 (m, 1H), 2.57- |
| | | | 2.48 (m, 1H), 2.24-2.13 (m, 6H), 2.02-2.01 (s, |
| | | | 1H), 2.00-1.98 (m, 6H), 1.96-1.91 (m, 1H), |
| | | | 1.60 (s, 6H), 1.46 (d, J = 7.0 Hz, 2H), 1.30 (d, |
| | | | J = 10.4 Hz, 2H), 1.25-1.23 (m, 1H), 1.11 (d, |
| | | | J = 6.5 Hz, 3H), 0.73 (dt, J = 7.4 Hz, 3H). |
| B133 | 985.5 | 985.7 | 1H NMR (400 MHz, MeOD) δ 8.39 (d, J = |
| | | | 8.4 Hz, 1H), 8.02 (d, J = 6.8 Hz, 1H), 7.85 (t, |
| | | | J = 8.4 Hz, 1H), 7.65 (d, J = 8.0 Hz, 2H), 7.49 |
| | | | (d, J = 8.4 Hz, 1H), 7.40 (s, 3H), 7.39-7.31 |
| | | | (m, 1H), 7.03 (d, J = 8.4 Hz, 1H), 5.50 (s, |
| | | | 1H), 5.07-4.99 (m, 1H), 4.59-4.51 (m, 1H), |
| | | | 4.45 (s, 1H), 3.85-3.83 (m, 1H), 3.78-3.70 (m, |
| | | | 1H), 3.45-3.43 (m, 4H), 3.33-3.31 (m, 2H), |
| | | | 2.96-2.94 (m, 4H), 2.75-2.74 (m, 1H), 2.68- |
| | | | 2.66 (m, 3H), 2.58 (d, J = 6.4 Hz, 1H), 2.39 |
| | | | (s, 3H), 2.22-2.20 (m, 1H), 2.14-2.11 (m, |
| | | | 2H), 2.05-1.93 (m, 3H), 1.81-1.79 (m, 1H), |
| | | | 1.63 (d, J = 7.2 Hz, 1H), 1.57 (s, 6H), 1.56- |
| | | | 1.54 (s, 1H), 1.52-1.50 (m, 2H), 1.22-1.20 (m, |
| | | | 2H), 1.07 (s, 9H). |
| B134 | 901.4 | 901.6 | 1H NMR (400 MHz, MeOD) δ 8.42 (d, J = |
| | | | 8.4 Hz, 1H), 8.08-7.93 (m, 3H), 7.93-7.79 (m, |
| | | | 2H), 7.66 (m, 2H), 7.42 (t, J = 10.4 Hz, 2H), |
| | | | 7.36 (d, J = 7.8 Hz, 1H), 5.28 (d, J = 10.4 Hz, |
| | | | 1H), 5.04 (d, J = 7.2 Hz, 1H), 4.52-4.49 (m, |
| | | | 2H), 3.99-3.73 (m, 4H), 3.53-3.50 (m, 1H), |
| | | | 3.21-3.16 (m, 5H), 2.81-2.79 (m, 1H), 2.58 (s, |
| | | | 3H), 2.25-2.23 (m, 2H), 2.15-2.10 (m, 5H), |
| | | | 2.08-1.92 (m, 4H), 1.63-1.61 (m, 1H), 1.60 (s, |
| | | | 6H), 1.58-1.57 (m, 1H), 1.50 (d, J = 7.0 Hz, |
| | | | 3H), 1.33-1.31 (m, 2H), 1.12 (d, J = 6.4 Hz, |
| | | | 3H), 0.75 (d, J = 6.8 Hz, 3H). |
| B135 | 1080.5 | 1080.7 | 1H NMR (400 MHz, MeOD) δ 8.99 (s, 1H), |
| | | | 8.46 (d, J = 8.4 Hz, 1H), 8.08 (d, J = 5.2 Hz, |
| | | | 1H), 7.88 (t, J = 8.4 Hz, 1H), 7.75-7.70 (m, |
| | | | 4H), 7.57 (d, J = 8.4 Hz, 1H), 7.45-7.43 (m, |
| | | | 2H), 7.10 (d, J = 8.4 Hz, 1H), 5.51 (s, 1H), |
| | | | 5.05-5.03 (m, 4H), 4.58-4.53 (m, 2H), 4.45- |
| | | | 4.43 (m, 4H), 4.02-3.98 (m, 3H), 3.85-3.83 |
| | | | (m, 1H), 3.79-3.77 (m, 6H), 3.50-3.48 (m, |
| | | | 1H), 3.20 (d, J = 6.4 Hz, 4H), 3.13 (s, 1H), |
| | | | 2.20-2.15 (m, 2H), 2.05-2.03 (m, 4H), 1.94- |
| | | | 1.93 (m, 1H), 1.65-1.63 (m, 1H), 1.60 (s, 6H), |
| | | | 1.54 (d, J = 7.2 Hz, 3H), 1.32-1.30 (mz, 2H), |
| | | | 1.08 (s, 9H), 0.95 (t, J = 7.2 Hz, 1H). |
| B136 | 1066.5 | 1066.7 | 1H NMR (400 MHz, MeOD) δ 8.94 (s, 1H), |
| | | | 8.41 (d, J = 8.8 Hz, 1H), 7.93-7.92 (m, 1H), |
| | | | 7.88-7.82 (m, 1H), 7.78 (d, J = 8.4 Hz, 2H), |
| | | | 7.71 (s, 1H), 7.67 (d, J = 8.0 Hz, 1H), 7.55 (d, |
| | | | J = 8.4 Hz, 1H), 7.42 (d, J = 9.6 Hz, 2H), 7.08 |
| | | | (d, J = 8.4 Hz, 1H), 5.28 (d, J = 10.4 Hz, 1H), |
| | | | 5.05-5.02 (m, 4H), 4, .54-4.52 (m, 1H), 4.48- |
| | | | 4.47 (m, 1H), 3.99-3.87 (m, 4H), 3.86-3.85 |
| | | | (m, 1H), 3.81-3.79 (m, 6H), 3.48-3.46 (m, |
| | | | 2H), 3.19 (d, J = 6.4 Hz, 2H), 3.13 (d, J = 1.6 |
| | | | Hz, 1H), 2.95-2.93 (m, 4H), 2.80-2.76 (m, |
| | | | 1H), 2.58-2.56 (m, 1H), 2.20-2.17 (m, 3H), |
| | | | 2.05-1.96 (m, 4H), 1.66-1.60 (m, 2H), 1.58 (s, |
| | | | 6H), 1.52 (d, J = 7.2 Hz, 3H), 1.35-1.33 (m, |
| | | | 2H), 1.14 (d, J = 6.4 Hz, 3H), 0.74 (d, J = 7.2 |
| | | | Hz, 3H). |
| B140 | 1120.5 | 561.0 | 1H NMR (400 MHz, MeOD) δ 8.90 (s, 1H), |
| | | | 8.38 (d, J = 8.7 Hz, 1H), 8.03 (d, J = 9.3 Hz, |
| | | | 1H), 7.85-7.74 (m, 4H), 7.64 (d, J = 7.5 Hz, |
| | | | 2H), 7.45-7.36 (m, 2H), 7.02 (d, J = 8.2 Hz, |
| | | | 1H), 5.51 (s, 1H), 5.06-5.02 (m, 1H), 4.58- |
| | | | 4.52 (m, 1H), 4.45 (s, 1H), 3.87-3.69 (m, 6H), |
| | | | 3.60-3.59 (m, 2H), 3.50-3.48 (m, 4H), 3.13- |
| | | | 3.04 (m, 4H), 2.76-2.61 (m, 7H), 2.21-1.99 |
| | | | (m, 10H), 1.89-1.72 (m, 7H), 1.65-1.53 (m, |
| | | | 5H), 1.24-1.19 (m, 2H), 1.07 (s, 9H). |
| B141 | 981.4 | 981.6 | 1H NMR (400 MHz, MeOD) δ 9.03 (s, 1H), |
| | | | 8.44 (d, J = 8.6 Hz, 1H), 8.03-7.96 (m, 2H), |
| | | | 7.85 (d, J = 8.3 Hz, 1H), 7.75 (t, J = 7.6 Hz, |
| | | | 2H), 7.64 (d, J = 7.9 Hz, 1H), 7.56 (d, J = 7.8 |
| | | | Hz, 1H), 7.51 (d, J = 8.3 Hz, 2H), 7.35 (t, J = |
| | | | 11.5 Hz, 1H), 5.49 (s, 1H), 5.08-5.06 (m, 1H), |
| | | | 4.57-4.54 (m, 1H), 4.46-4.45 (m, 1H), 3.85- |
| | | | 3.83 (m, 1H), 3.64-3.46 (m, 1H), 3.17-3.15 |
| | | | (m, 2H), 2.81-2.68 (m, 2H), 2.37-2.35 (m, |
| | | | 2H), 2.30-2.15 (m, 4H), 2.11-2.09 (m, 2H), |
| | | | 2.02-1.89 (m, 8H), 1.59 (s, 6H), 1.55 (d, J = |
| | | | 7.0 Hz, 3H), 1.35-1.31 (m, 6H), 1.07 (s, 9H). |
| B143 | 993.4 | 497.4 | 1H NMR (400 MHz, MeOD) δ 9.00 (s, 1H), |
| | | | 8.50-8.48 (m, 3H), 8.12 (d, J = 24.5 Hz, 2H), |
| | | | 8.00 (s, 1H), 7.87 (d, J = 8.2 Hz, 1H), 7.81- |
| | | | 7.72 (m, 2H), 7.61-7.67 (m, 2H), 7.36 (d, J = |
| | | | 7.7 Hz, 1H), 5.58 (s, 1H), 4.60-4.57 (m, 9H), |
| | | | 3.69-3.67 (m, 2H), 3.08-3.05 (m, 5H), 2.60 (s, |
| | | | 3H), 2.17-2.15 (m, 6H), 2.04-2.02 (m, 3H), |
| | | | 1.63-1.60 (m, 8H), 1.10 (s, 9H). |
| B146 | 996.5 | 996.8 | 1H NMR (400 MHz, MeOD) δ 8.87-8.86 (m, |
| | | | 1H), 8.63-8.52 (m, 1H), 8.44 (d, J = 8.4 Hz, |
| | | | 1H), 8.08-8.01 (m, 1H), 7.97 (s, 1H), 7.87 (t, |
| | | | J = 8.4 Hz, 1H), 7.66 (d, J = 8.0 Hz, 1H), 7.57 |
| | | | (d, J = 7.6 Hz, 1H), 7.34 (d, J = 7.6 Hz, 1H), |
| | | | 7.28-7.22 (m, 2H), 5.52 (d, J = 7.2 Hz, 1H), |
| | | | 5.11-5.09 (m, 1H), 4.60-4.58 (m, 1H), 4.53- |
| | | | 4.44 (m, 2H), 3.94-3.92 (m, 1H), 3.77 (d, J = |
| | | | 11.2 Hz, 1H), 3.20-3.18 (m, 2H), 2.84-2.80 |
| | | | (m, 4H), 2.74-2.72 (m, 1H), 2.47 (s, 3H), |
| | | | 2.45-2.42 (m, 1H), 2.24-2.22 (m, 1H), 2.18- |
| | | | 2.16 (m, 1H), 2.12-2.10 (m, 4H), 1.98-1.90 |
| | | | (m, 8H), 1.76-1.75 (m, 1H), 1.68-1.67 (m, |
| | | | 1H), 1.59 (s, 6H), 1.55-1.45 (m, 2H), 1.24- |
| | | | 1.14 (m, 2H), 1.09-1.03 (m, 9H). |
| B148 | 982.4 | 982.4 | 1H NMR (400 MHz, MeOD) δ 8.95 (s, 1H), |
| | | | 8.42 (t, J = 4.2 Hz, 2H), 8.02-8.00 (m, 2H), |
| | | | 7.94-7.81 (m, 2H), 7.68-7.61( m, 2H), 7.52- |
| | | | 7.47 (m, 1H), 7.38-7.34 (m, 1H), 4.58-4.56 |
| | | | (m, 1H), 4.02-4.00 (m, 1H), 3.80-3.73 (m, |
| | | | 2H), 3.15-3.09 (m, 6H), 2.53 (s, 3H), 2.19- |
| | | | 2.14 (m, 6H), 2.04-1.96 (m, 3H), 1.96-1.94 |
| | | | (m, 2H), 1.59 (s, 6H), 1.33-1.25 (m, 5H), 1.11 |
| | | | (s, 9H), 1.02-1.00 (m, 2H). |
| B149 | 984.5 | 492.8 | 1H NMR (400 MHz, MeOD) δ 8.86 (s, 1H), |
| | | | 8.43 (d, J = 8.5 Hz, 1H), 8.05-8.03 (m, 2H), |
| | | | 7.86 (t, J = 8.3 Hz, 1H), 7.72-7.57 (m, 2H), |
| | | | 7.44-7.20 (m, 4H), 5.48 (s, 1H), 5.27-5.20 (m, |
| | | | 1H), 4.60-4.57 (m, 4H), 4.46-4.45 (m, 1H), |
| | | | 3.85-3.83 (m, 1H), 3.75-3.73 (m, 1H), 3.55- |
| | | | 3.53 (m, 2H), 3.04-3.02 (m, 1H), 2.91-2.89 |
| | | | (m, 3H), 2.78-2.76 (m, 1H), 2.48 (s, 3H), |
| | | | 2.44-2.42 (m, 2H), 2.38-2.37 (m, 1H), 2.16- |
| | | | 2.13 (m, 6H), 2.01-1.96 (m, 4H), 1.59 (s, 6H), |
| | | | 1.56-1.55 (m, 1H), 1.51-1.49 (m, 3H), 1.27- |
| | | | 1.25 (m, 2H), 1.10 (s, 9H). |
| B151 | 996.5 | 996.1 | 1H NMR (400 MHz, MeOD) δ 8.86 (d, J = |
| | | | 4.9 Hz, 1H), 8.43 (d, J = 8.7 Hz, 1H), 8.10 (s, |
| | | | 1H), 7.99 (s, 1H), 7.86 (t, J = 8.3 Hz, 1H), |
| | | | 7.72-7.57 (m, 3H), 7.35 (d, J = 7.8 Hz, 1H), |
| | | | 7.30-7.26 (m, 2H), 5.53 (s, 1H), 5.15-5.12 (m, |
| | | | 1H), 4.52-4.50 (m, 2H), 3.93 (d, J = 10.9 Hz, |
| | | | 1H), 3.81-3.79 (m, 1H), 3.52-3.49 (m, 2H), |
| | | | 2.93-2.80 (m, 7H), 2.51-2.44 (m, 3H), 2.24- |
| | | | 1.79 (m, 15H), 1.63-1.51 (m, 8H), 1.35-1.21 |
| | | | (m, 3H), 1.13-1.03 (m, 9H). |
| B156 | 1079.5 | 540.4 | 1H NMR (400 MHz, MeOD) δ 8.90 (s, 1H), |
| | | | 8.44 (d, J = 8.6 Hz, 1H), 8.03-7.97 (m, 2H), |
| | | | 7.87 (t, J = 8.3 Hz, 1H), 7.79 (d, J = 7.7 Hz, |
| | | | 2H), 7.65-7.63 (m, 2H), 7.52-7.36 (m, 3H), |
| | | | 6.30 (s, 1H), 5.50 (s, 1H), 5.04-4.99 (m, 1H), |
| | | | 4.61-4.52 (m, 3H), 4.42-4.40 (m, 1H), 3.85- |
| | | | 3.82 (m, 1H), 3.76-3.72 (m, 5H), 3.61-3.56 |
| | | | (m, 2H), 3.25-3.23 (m, 2H), 2.80-2.78 (m, |
| | | | 2H), 2.72-2.70 (s, 3H), 2.65-2.59 (m, 4H), |
| | | | 2.40 (d, J = 6.9 Hz, 2H), 2.17-2.15 (m, 3H), |
| | | | 2.06-1.89 (m, 4H), 1.60 (s, 6H), 1.55-1.51 (m, |
| | | | 5H), 1.20-1.16 (m, 2H), 1.07 (d, J = 3.5 Hz, |
| | | | 9H). |
| B164 | 984.5 | 984.7 | 1H NMR (400 MHz, MeOD-d4) δ 8.44 (d, |
| | | | J = 8.4 Hz, 1H), 8.09-7.94 (m, 2H), 7.92-7.84 |
| | | | (m, 1H), 7.66 (d, J = 8.0 Hz, 1H), 7.57 (d, J = |
| | | | 7.6 Hz, 1H), 7.40-7.39 (m, 4H), 7.33 (s, 1H), |
| | | | 5.50 (s, 1H), 5.12-4.97 (m, 1H), 4.61-4.49 (m, |
| | | | 1H), 4.42 (m, 1H), 3.85 (m, 1H), 3.80-3.63 |
| | | | (m, 1H), 3.15-3.14 (m, 2H), 2.87-2.71 (m, |
| | | | 2H), 2.65 (d, J = 2.4 Hz, 3H), 2.39 (d, J = 2.8 |
| | | | Hz, 3H), 2.35-2.33 (m, 2H), 2.23-2.19 (m, |
| | | | 3H), 2.13-2.10 (m, 2H), 2.02-1.99 (m, 3H), |
| | | | 1.96-1.88 (m, 5H), 1.73-1.62 (m, 1H), 1.59 (s, |
| | | | 6H), 1.55-1.52 (m, 4H), 1.20-1.17 (m, 2H), |
| | | | 1.07 (s, 9H). |
| B168 | 1025.5 | 513.4 | 1H NMR (400 MHz, MeOD-d4) δ 8.37 (d, |
| | | | J = 8.4 Hz, 1H), 8.03 (s, 1H), 7.93-7.71 (m, |
| | | | 2H), 7.65-7.64 (m, 2H), 7.49-7.33 (m, 4H), |
| | | | 7.02 (d, J = 8.4 Hz, 1H), 5.50 (s, 1H), 5.04- |
| | | | 5.02 (m, 2H), 4.62-4.54 (m, 3H), 4.45-4.43 |
| | | | (m, 1H), 3.88-3.86 (m, 1H), 3.80-3.70 (m, |
| | | | 1H), 3.53-3.50 (m, 4H), 3.18-3.15 (m, 3H), |
| | | | 2.80-2.78 (m, 2H), 2.66 (d, J = 2.4 Hz, 3H), |
| | | | 2.39 (d, J = 3.2 Hz, 3H), 2.21-2.19 (m, 2H), |
| | | | 2.11-1.95 (m, 7H), 1.93-1.90 (m, 4H), 1.75- |
| | | | 1.72 (m, 3H), 1.65-1.56 (m, 2H), 1.53 (d, J = |
| | | | 7.2 Hz, 3H), 1.32-1.21 (m, 2H), 1.07 (s, 9H). |

### Compound B109. (2S,4R)-1-((S)-2-((1r,4S)-4-((4-(4-chloro-7,7-dimethyl-5-oxo-5,7-dihydroindolo[1,2-a]quinazolin-9-yl)piperidin-1-yl)methyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide and Compound B110. (2S,4R)-1-(2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoyl)-4-hydroxy-N—((R)-1-(4-(4-methylthiazol-5-yl)phenyl)-2-morpholinoethyl)pyrrolidine-2-carboxamide

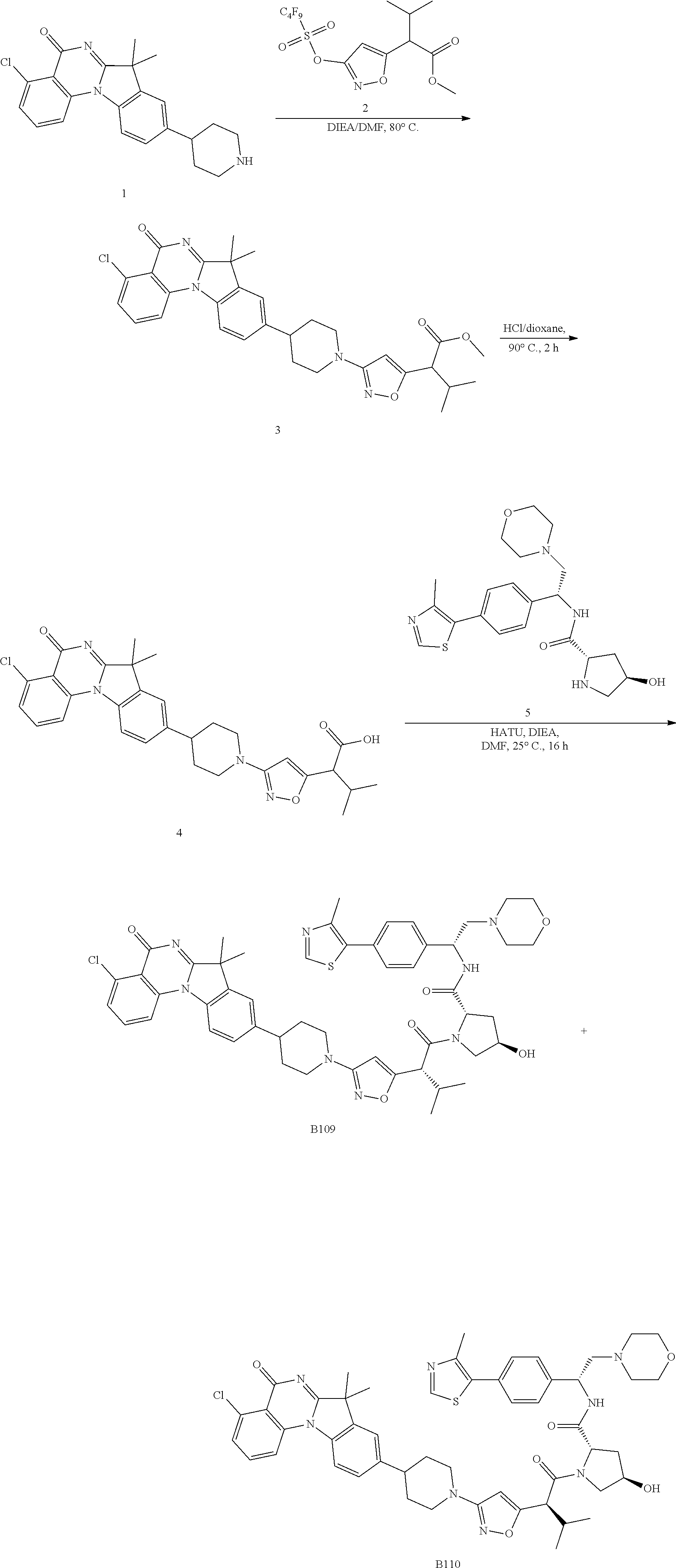

### Synthesis of tert-butyl methyl2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoate (Intermediate 3)

To a solution of 4-chloro-7,7-dimethyl-9-(piperidin-4-yl)indolo[1,2-a]quinazolin-5 (7H)-one (200 mg, 1 eq, 526 μmol) in DMF (2 mL) were added N-ethyl-N-isopropylpropan-2-amine (340 mg, 5 eq, 2.63 mmol) and methyl 3-methyl-2-(3-(((perfluorobutyl) sulfonyl)oxy)isoxazol-5-yl)butanoate (304 mg, 1.2 Eq, 632 μmol). The mixture was stirred at 80° C. for 16 h under N₂. LCMS indicated completion of reaction. The reaction was concentrated under vacuum and purified by prep-HPLC, eluted with MeCN in H2O (0.1% FA) to afford methyl 2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoate (60.0 mg, 0.10 mmol, 19%) as yellow oil. LC purity (0.1% FA): 91.98% (UV at 254 nm)/MS: 561.08 [M+H]; Retention time: 1.61 min.

### Synthesis of tert-butyl 2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoic acid (Intermediate 4)

To a solution of methyl 2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoate (200 mg, 1 eq, 356 μmol) in HCl (1 mL), water (1 mL) and 1,4-dioxane (1 mL). The mixture was stirred at 90° C. for 1 h under N2. LCMS showed the reaction was complete. The reaction was concentrated under vacuum and purified by prep-HPLC, eluted with MeCN in H₂O (0.1% FA) to afford 2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoic acid (40.0 mg, 73.1 μmol, 20.5%) as yellow oil. LC purity (10 mmol/L NH₄HCO₃ in H₂O): 100% (UV at 254 nm)/MS: 547.05 [M−56]; Retention time: 1.29 min.

### Synthesis of (2S,4R)-1-(2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoyl)-4-hydroxy-N—((R)-1-(4-(4-methylthiazol-5-yl)phenyl)-2-morpholinoethyl)pyrrolidine-2-carboxamide (B109) and (2S,4R)-1-(2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoyl)-4-hydroxy-N—((R)-1-(4-(4-methylthiazol-5-yl)phenyl)-2-morpholinoethyl)pyrrolidine-2-carboxamide (B110)

To a solution of 2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoic acid (20.0 mg, 1 eq, 36.6 μmol) in DMF (0.3 mL) were added N-ethyl-N-isopropylpropan-2-amine (18.9 mg, 4 eq, 146 μmol), HATU (27.8 mg, 2 eq, 73.1 μmol) and (2S,4R)-4-hydroxy-N—((R)-1-(4-(4-methylthiazol-5-yl)phenyl)-2-morpholinoethyl)pyrrolidine-2-carboxamide (15.2 mg, 1 eq, 36.6 μmol). The mixture was stirred at 25° C. for 4 h. LCMS indicated completion of reaction. The reaction was concentrated and purified by prep-HPLC, eluted with CH₃CN in H₂O (0.1% FA) from 5.0% to 95.0% to give (2S,4R)-1-(2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoyl)-4-hydroxy-N—((R)-1-(4-(4-methylthiazol-5-yl)phenyl)-2-morpholinoethyl)pyrrolidine-2-carboxamide (4.22 mg, 3.59 μmol, 9.81%) as a white solid. LC purity (0.1% FA): 96.65% (UV at 254 nm)/MS: 945.4 [M+H];

Retention time: 1.38 min. ¹H NMR (400 MHz, MeOD) δ 8.88-8.86 (m, 1H), 8.41 (d, J=8.4 Hz, 2H), 8.05 (d, J=8.5 Hz, 1H), 7.86-7.82 (m, 1H), 7.65 (d, J=8.1 Hz, 1H), 7.57-7.52 (m, 1H), 7.45-7.40 (m, 2H), 7.36 (d, J=7.9 Hz, 2H), 6.29-6.21 (m, 1H), 5.15-5.03 (m, 1H), 4.68-4.59 (m, 1H), 4.52-4.50 (m, 1H), 3.86-3.70 (m, 9H), 3.10-2.94 (m, 3H), 2.79-2.51 (m, 7H), 2.50-2.44 (m, 3H), 2.28-2.23 (m, 1H), 2.09-1.99 (m, 1H), 1.99-1.75 (m, 4H), 1.61-1.57 (m, 6H), 1.09 (d, J=6.6 Hz, 2H), 0.96 (d, J=6.7 Hz, 3H) and (2S,4R)-1-(2-(3-(4-(4-chloro-7,7-dimethyl-5-oxo-5,7-dihydroindolo[1,2-a]quinazolin-9-yl)piperidin-1-yl)isoxazol-5-yl)-3-methylbutanoyl)-4-hydroxy-N—((R)-1-(4-(4-methylthiazol-5-yl)phenyl)-2-morpholinoethyl)pyrrolidine-2-carboxamide (3.43 mg, 3.06 μmol, 8.37%) as a white solid. LC purity (0.1% FA): 96.65% (UV at 254 nm)/MS: 945.58 [M+H]; Retention time: 1.38 min. ¹H NMR (400 MHz, MeOD) δ 8.88-8.86 (m, 1H), 8.46-8.36 (m, 1H), 8.08-8.04 (m, 1H), 7.86-7.82 (m, 1H), 7.66-7.64 (m, 1H), 7.57 (s, 1H), 7.46-7.35 (m, 4H), 6.16-6.14 (m, 1H), 5.16-5.12 (m, 1H), 4.58-4-56 (m, 1H), 4.48-4.46 (m, 1H), 3.89-3.60 (m, 9H), 3.12-3.02 (m, 3H), 2.89-2.60 (m, 7H), 2.49-2.44 (m, 3H), 2.27-2.18 (m, 1H), 2.03-1.79 (m, 5H), 1.61-1.59 (m, 6H), 1.10-1.05 (m, 3H), 1.01-0.88 (m, 3H).

The examples in the table below were prepared according to the same method as example B109 using appropriate starting materials

| | Calcd. | Found | |
|---|---|---|---|
| Ex. | (M + H)+ m/z | (M + H)+ m/z | HNMR |
| B111 | 958.4 | 479.8 | 1H NMR (400 MHz, MeOD) δ 8.87 (s, 1H), |
| | | | 8.39 (d, J = 8.5 Hz, 1H), 7.85 (t, J = 8.2 Hz, |
| | | | 1H), 7.67-7.65 (m, 2H), 7.35-7.51 (m, 5H), |
| | | | 7.04 (d, J = 8.4 Hz, 1H), 6.11 (s, 1H), 5.02- |
| | | | 5.00 (mz, 1H), 4.60-4.58 (m, 2H), 4.43-4.41 |
| | | | (m, 1H), 3.58-3.75 (m, 4H), 3.46-3.44 (s, |
| | | | 5H), 2.88-2.86 (m, 2H), 2.70-2.65 (m, 4H), |
| | | | 2.48 (s, 3H), 2.47-2.45 (m, 2H), 1.96-1.94 (m, |
| | | | 1H), 1.88-1.85 (m, 3H), 1.57 (s, 6H), 1.53- |
| | | | 1.51 (m, 3H), 1.31-1.29 (m, 3H), 1.06 (d, J = |
| | | | 6.6 Hz, 3H), 0.86-0.94 (m, 3H). |
| B115 | 1042.5 | 1042.5 | 1H NMR (400 MHz, MeOD) δ 8.87 (s, 1H), |
| | | | 8.44 (d, J = 8.7 Hz, 1H), 7.97 (s, 1H), 7.88 (t, |
| | | | J = 8.3 Hz, 1H), 7.66 (d, J = 7.9 Hz, 1H), 7.56 |
| | | | (d, J = 7.7 Hz, 1H), 7.28-7.49 (m, 5H), 6.10 |
| | | | (s, 1H), 5.10-5.09 (m, 1H), 4.56 (t, J = 7.9 Hz, |
| | | | 1H), 4.41-4.40 (m, 1H), 3.87-3.85 (m, 1H), |
| | | | 3.72-3.70 (m, 6H), 3.61-3.59 (m, 2H), 3.11 |
| | | | (d, J = 11.5 Hz, 2H), 2.89-2.87 (m, 2H), 2.77- |
| | | | 2.75 (m, 2H), 2.68-2.65 (m, 1H), 2.55 (d, J = |
| | | | 12.1 Hz, 3H), 2.48 (d, J = 2.3 Hz, 3H), 2.40- |
| | | | 2.39 (m, 1H), 2.31 (d, J = 6.4 Hz, 2H), 2.20- |
| | | | 2.18 (m, 3H), 1.99-1.80 (m, 8H), 1.59 (s, 6H), |
| | | | 1.32-1.30 (m, 3H), 1.08-1.06 (m, 3H), 0.92 |
| | | | (d, J = 15.7 Hz, 3H). |
| B142 | 958.4 | 479.8 | 1H NMR (400 MHz, MeOD) δ 8.87 (s, 1H), |
| | | | 8.38 (d, J = 8.6 Hz, 1H), 7.84 (t, J = 8.3 Hz, |
| | | | 1H), 7.62 (dd, J = 18.4, 4.6 Hz, 2H), 7.49- |
| | | | 7.39 (m, 5H), 7.01 (dd, J = 8.5, 1.5 Hz, 1H), |
| | | | 6.07 (s, 1H), 5.07-5.04 (m, 1H), 4.59-4.51 (m, |
| | | | 2H), 4.45-4.44 (m, 1H), 3.85 (dd, J = 10.8, |
| | | | 4.2 Hz, 1H), 3.73-3.58 (m, 4H), 3.33 (d, J = |
| | | | 5.1 Hz, 4H), 2.87 (t, J = 12.0 Hz, 2H), 2.70- |
| | | | 2.68 (m, 4H), 2.48 (s, 3H), 2.37 (s, 3H), 2.21- |
| | | | 2.14 (m, 1H), 1.99-1.97 (m, 1H), 1.87 (d, J = |
| | | | 11.7 Hz, 3H), 1.56 (s, 6H), 1.52 (d, J = 7.0 |
| | | | Hz, 2H), 1.31-1.29 (m, 2H), 1.05 (d, J = 6.5 |
| | | | Hz, 3H), 0.90 (d, J = 6.0 Hz, 3H). |
| B160 | 1083.5 | 1083.9 | 1H NMR (400 MHz, MeOD) δ 8.87 (s, 1H), |
| | | | 8.37 (d, J = 8.6 Hz, 1H), 7.86-7.81 (m, 1H), |
| | | | 7.75 (d, J = 8.5 Hz, 1H), 7.66-7.59 (m, 2H), |
| | | | 7.48-7.40 (m, 4H), 6.99 (d, J = 8.7 Hz, 1H), |
| | | | 6.11-6.09 (m, 1H), 5.13-5.05 (m, 1H), 4.57- |
| | | | 4.53 (m, 1H), 4.44-4.38 (m, 1H), 3.86-3.48 |
| | | | (m, 11H), 2.92-2.76 (m, 4H), 2.73-2.53 (m, |
| | | | 10H), 2.48 (s, 3H), 2.37-2.36 (m, 3H), 2.09- |
| | | | 1.98 (m, 5H), 1.89-1.86 (m, 7H), 1.75-1.70 |
| | | | (m, 3H), 1.31-1.29 (m, 2H), 1.1.04-1.03 (m, |
| | | | 3H), 0.90-0.88 (m, 3H). |
| B167 | 874.4 | 874.5 | 1H NMR (400 MHz, MeOD-d4) δ 8.45 (d, J = |
| | | | 8.7 Hz, 1H), 7.99 (d, J = 7.1 Hz, 1H), 7.83 |
| | | | (t, J = 8.3 Hz, 1H), 7.66 (d, J = 7.8 Hz, 1H), |
| | | | 7.55-7.44 (m, 1H), 7.43-7.29 (m, 5H), 6.14 (s, |
| | | | 1H), 5.05-5.03 (m, 1H), 4.60-4.39 (m, 2H), |
| | | | 3.92-3.79 (m, 3H), 3.70-3.50 (m, 2H), 3.11- |
| | | | 3.09 (m, 2H), 2.65 (s, 3H), 2.42-2.36 (m, 4H), |
| | | | 2.20-2.18 (m, 1H), 2.04-1.93 (m, 3H), 1.88- |
| | | | 1.86 (m, 2H), 1.75 (s, 6H), 1.62-1.48 (m, 3H), |
| | | | 1.07 (d, J = 6.5 Hz, 3H), 0.99-0.89 (m, 3H). |

### Compound B132. (2S,4R)-1-((S)-2-(4-((4-(4-(4-chlors-7,7-dimethyl-5-exe-5,7-dihydroidolo[1,2-a]quinazolin-8-yl)piperidin-1-yi)cyclohexyl)methyl)-1H-1,2,3-triazol-1-yl)-3-methylbutanoyl)-4-hydroxy-N—(S)-1-(4-(4-methylthiazol-5-yl)phenyl)ethyl)pyrrolidine-2-carboxamide

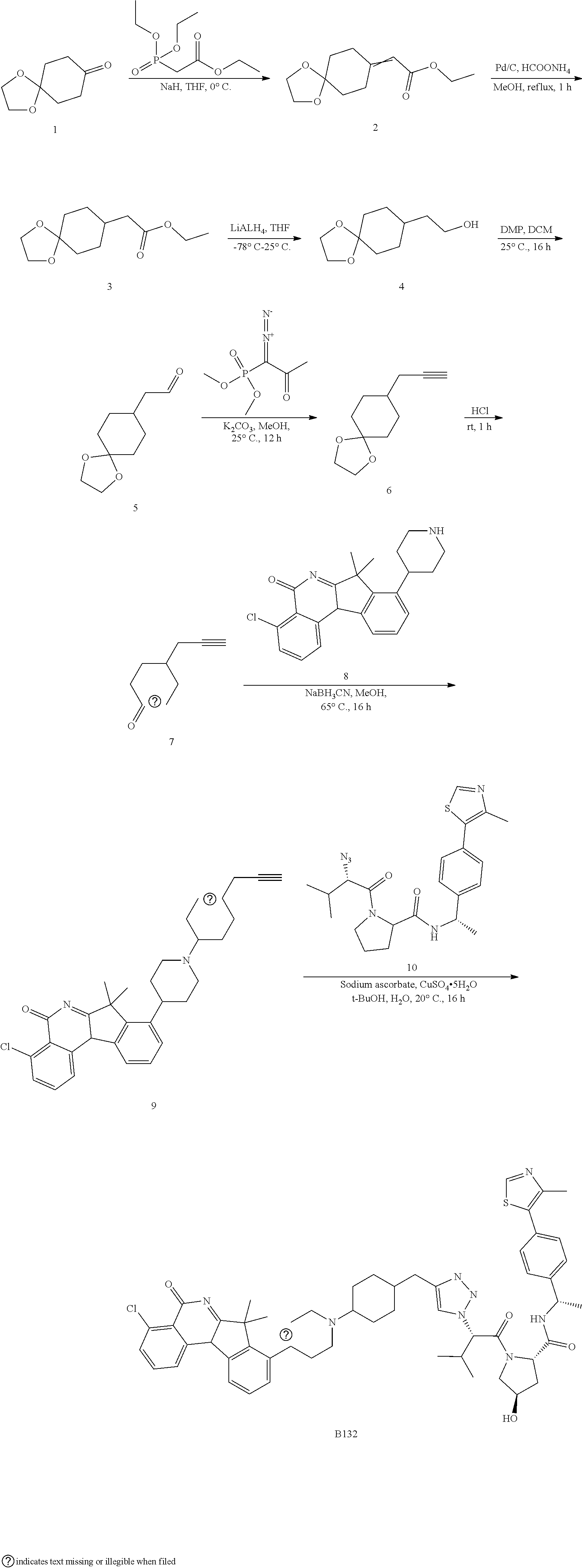

### Synthesis of ethyl 2-(1,4-dioxaspiro[4.5]decan-8-ylidene)acetate (Intermediate 2)

To a solution of ethyl 2-(diethoxyphosphoryl)acetate (1.72 g, 1.52 mL, 1.2 eq, 7.68 mmol) in THF (20 mL). Then was added NaH (307 mg, 2 eq, 12.8 mmol) at 0° C. The temperature of the resulting mixture was kept at this temperature 15 min. To the resulting mixture was added 1,4-dioxaspiro[4.5]decan-8-one (1.00 g, 1 eq, 6.40 mmol). The reaction mixture was stirred 25° C. for 2 h. TLC indicated completion of reaction. The resulting mixture was quenched with water and extracted with EA (20 mL). The organic layer was washed with water (20 mL×2) and brine (30 mL). The organic layer was dried over sodium sulfate, filtered and concentrated under vacuum. The resulting residue was then purified by silica gel column (100-200 mesh silica gel, 30% of EA in PE) to afford ethyl 2-(1,4-dioxaspiro[4.5]decan-8-ylidene)acetate (1.30 g, 5.75 mmol, 89.7%) as a yellow oil.

### Synthesis of ethyl 2-(1,4-dioxaspiro[4.5]decan-8-yl)acetate (Intermediate 3)

To a solution of ethyl 2-(1,4-dioxaspiro[4.5]decan-8-ylidene)acetate (300 mg, 1 eq, 1.33 mmol) in methanol (2 mL) was added Pearlman's catalyst (42.3 mg, 0.3 eq, 0.40 mmol) and ammonium formate (167 mg, 0.13 mL, 2 eq, 2.65 mmol). The mixture was stirred at 75° C. for 1 h. TLC indicated completion of reaction. The reaction was poured into water (15 mL) and extracted with EA (30 ml). The organic layer was washed brine, dried over Na₂SO₄, filtered and concentrated to get crude ethyl 2-(1,4-dioxaspiro[4.5]decan-8-yl)acetate (260 mg, 1.0 mmol, 77%) as yellow oil. ¹H NMR (400 MHz, DMSO) δ 4.03-4.05 (t, J=7.2 Hz, 2H), 3.83 (s, 4H), 2.50-2.51 (m, 1H), 2.18-2.20 (d, J=7.2 Hz, 2H), 1.60-1.71 (m, 6H), 1.41-1.49 (m, 2H), 1.13-1.19 (m, 3H).

### Synthesis of 2-(1,4-dioxaspiro[4.5]decan-8-yl) ethan-1-ol (Intermediate 4)

To a solution of ethyl 2-(1,4-dioxaspiro[4.5]decan-8-yl)acetate (860 mg, 1 eq, 3.77 mmol) in THF (5 mL) was added LiAlH₄ (143 mg, 5.00 mL, 1 eq, 3.77 mmol) at 0° C. The mixture was stirred at 25° C. for 4 h. TLC indicated completion of reaction. The reaction mixture was poured into water (15 mL) and extracted with EA (30 mL). The organic layer was washed with brine, dried over Na₂SO₄, filtered and concentrated to get crude 2-(1,4-dioxaspiro[4.5]decan-8-yl) ethan-1-ol (700 mg, 3.4 mmol, 90%) as a yellow oil. ¹H NMR (400 MHz, DMSO) δ 4.30-4.32 (t, J=5.2 Hz, 1H), 3.80-3.84 (d, J=7.6 Hz, 4H), 3.40-3.45 (m, 2H), 1.62-1.65 (d, J=10.0 Hz, 4H), 1.31-1.47 (m, 5H), 1.10-1.19 (m, 2H).

### Synthesis of 2-(1,4-dioxaspiro[4.5]decan-8-yl) acetaldehyde (Intermediate 5)

To a solution of 2-(1,4-dioxaspiro[4.5]decan-8-yl) ethan-1-ol (800 mg, 1 eq, 4.30 mmol) in DCM (3 mL) was added DMP (2.19 g, 1.2 eq, 5.15 mmol) at 0° C. The mixture was stirred at 25° C. for 3 h. TLC indicated completion of reaction. The reaction was poured into water (15 mL) and extracted with EA (30 mL). The organic layer was washed brine, dried over Na₂SO₄, filtered and concentrated to get crude 2-(1,4-dioxaspiro[4.5]decan-8-yl) acetaldehyde (790.0 mg, 3.9 mmol, 90%) as a yellow oil. ¹H NMR (400 MHz, DMSO) δ 9.65-9.67 (t, J=2.0 Hz, 1H), 3.85 (s, 4H), 2.30-2.35 (m, 2H), 1.62-1.64 (d, J=10.4 Hz, 6H), 1.40-1.44 (m, 1H), 1.14-1.21 (m, 2H).

### Synthesis of 8-(prop-2-yn-1-yl)-1,4-dioxaspiro[4.5]decane (Intermediate 6)

To a solution of 2-(1,4-dioxaspiro[4.5]decan-8-yl) acetaldehyde (100 mg, 1 eq, 0.54 mmol) in methanol (2 mL) were added dimethyl (1-diazo-2-oxopropyl)phosphonate (209 mg, 2 eq, 1.09 mmol) and K₂CO₃ (225 mg, 3 eq, 1.63 mmol). The mixture was stirred at 25° C. for 16 h. TLC indicated completion of reaction. The reaction was poured into water (15 mL) and extracted with EA (30 mL). The organic layer was washed brine, dried over Na₂SO₄, filtered and concentrated to purification (PE:EA=3:1) to get 8-(prop-2-yn-1-yl)-1,4-dioxaspiro[4.5]decane (55.0 mg, 0.29 mmol, 53%) as white oil. ¹H NMR (400 MHz, DMSO) δ 3.83 (s, 4H), 2.75-2.78 (t, J=2.8 Hz, 1H), 2.05-2.09 (m, 2H), 1.64-1.73 (m, 4H), 1.41-1.46 (m, 3H), 1.22-1.28 (m, 2H).

### Synthesis of 4-(prop-2-yn-1-yl)cyclohexan-1-one (Intermediate 7)

To a solution of 8-(prop-2-yn-1-yl)-1,4-dioxaspiro[4.5]decane (100 mg, 1 eq, 0.55 mmol) in water (3 mL) was added hydrogen chloride (202.3 mg, 10 eq, 5.55 mmol) at rt. The mixture was stirred at 25° C. for 1.5 h. TLC indicated completion of reaction. To the reaction mixture was added 1 N NaHCO₃ to adjust the pH to 6-7. The reaction was poured into water (15 mL) and extracted with EA (30 mL). The organic layer was washed with brine, dried over Na₂SO₄, filtered and concentrated to get crude 4-(prop-2-yn-1-yl)cyclohexan-1-one (40 mg, 0.27 mmol, 49%) as a yellow oil.

### Synthesis of 4-chloro-7,7-dimethyl-8-(1-(4-(prop-2-yn-1-yl)cyclohexyl)piperidin-4-yl)indolo[1,2-a]quinazolin-5 (7H)-one (Intermediate 9)

To a solution of 4-(prop-2-yn-1-yl)cyclohexan-1-one (70.0 mg, 1 eq, 0.51 mmol) in methanol (2 mL) were added 4-chloro-7,7-dimethyl-8-(piperidin-4-yl)indolo[1,2-a]quinazolin-5 (7H)-one (215 mg, 1.1 eq, 0.57 mmol), TEA (156 mg, 0.22 mL, 3 eq, 1.54 mmol) and sodium cyanoborohydride (48.4 mg, 0.045 mL, 1.5 eq, 0.77 mmol). The mixture was stirred at 60° C. for 16 h. LCMS indicated completion of reaction. The reaction was poured into H₂O (15 mL) and extracted with FA (30 mL). The organic layer was washed brine, dried over Na₂SO₄, filtered and concentrated. The crude product was purified by silica gel prep-TLC (CH₃OH/DCM=1/10) to get 4-chloro-7,7-dimethyl-8-(1-(4-(prop-2-yn-1-yl)cyclohexyl)piperidin-4-yl)indolo[1,2-a]quinazolin-5 (7H)-one (120.0 mg, 0.24 mmol, 46.7%) as a yellow solid. LC purity (0.1% FA): 97.27% (UV at 254 nm)/MS: 500.2 [M+H]; Retention time: 1.36 min.

### Synthesis of (2S,4R)-1-((S)-2-(4-((4-(4-(4-chloro-7,7-dimethyl-5-oxo-5,7-dihydroindolo[1,2-a]quinazolin-8-yl)piperidin-1-yl)cyclohexyl)methyl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide (B132)

To a solution of 4-chloro-7,7-dimethyl-8-(1-(4-(prop-2-yn-1-yl)cyclohexyl)piperidin-4-yl)indolo[1,2-a]quinazolin-5 (7H)-one (80.0 mg, 1 eq, 0.16 mmol) in THF (4 mL) and water (4 mL) were added (2S,4R)-1-((S)-2-azido-3-methylbutanoyl)-4-hydroxy-N—((S)-1-(4-(4-methylthiazol-5-yl)phenyl)ethyl)pyrrolidine-2-carboxamide (87.6 mg, 1.2 eq, 0.19 mmol), copper (II) sulfate pentahydrate (20.0 mg, 0.5 eq, 0.08 mmol and sodium 2-(1,2-dihydroxyethyl)-4-hydroxy-5-oxo-2,5-dihydrofuran-3-olate (15.8 mg, 0.5 eq, 0.08 mmol). The mixture was stirred at 25° C. for 16 h. LCMS indicated completion of reaction. The reaction was poured into water (15 mL) and extracted with EA (30 ml), the organic layer was washed brine, dried over Na₂SO₄, filtered and concentrated. The crude product was purified by silica gel prep-TLC (CH₃OH/DCM=1/10) to get (2S,4R)-1-((S)-2-(4-((4-(4-(4-chloro-7,7-dimethyl-5-oxo-5,7-dihydroindolo[1,2-a]quinazolin-8-yl)piperidin-1-yl)cyclohexyl)methyl)-1H-1,2,3-triazol-1-yl)-3-methylbutanoyl)-4-hydroxy-N—((S)-1-(4-(4-methylthiazol-5-yl)phenyl)ethyl)pyrrolidine-2-carboxamide (2.64 mg, 2.32 μmol, 1.45%) as a white solid. LC purity (0.03% TFA): 90.96% (UV at 254 nm)/MS: 956.6 [M+H]; Retention time: 1.387 min. ¹H NMR (400 MHz, DMSO) δ 8.99 (s, 1H), 8.43-8.43 (m, 1H), 8.03-8.00 (m, 1H), 7.82-7.80 (m, 1H), 7.66-7.64 (m, 1H), 7.60-7.55 (m, 1H), 7.45 (d, J=8.4 Hz, 1H), 7.40-7.38 (m, 2H), 7.28-7.26 (m, 1H), 6.90 (s, 1H), 6.72-6.70 (m, 1H), 5.26-5.24 (m, 1H), 4.38-4.36 (m, 1H), 3.98-3.96 (m, 2H), 3.52-3.51 (m, 1H), 2.76-2.74 (m, 3H), 2.68-2.66 (m, 2H), 2.48-2.46 (m, 2H), 2.33-2.31 (m, 1H), 2.20-2.18 (m, 2H), 2.00-1.99 (m, 1H), 1.69-1.67 (m, 3H), 1.53-1.51 (m, 2H), 1.33 (s, 6H), 1.33-1.30 (m, 4H), 1.26 (s, 6H), 1.07-1.05 (m, 2H), 0.85-0.88 (d, J=3.2 Hz, 8H), 0.65-0.66 (d, J=6.4 Hz, 2H).

The examples in the table below were prepared according to the same method as example B132 using appropriate starting materials

| | Calcd. | Found | |
|---|---|---|---|
| Ex. | (M + H)+ m/z | (M + H)+ m/z | HNMR |
| B114 | 942.4 | 471.8 | 1H NMR (400 MHz, MeOD) δ 9.03 (s, 1H), |
| | | | 8.45 (d, J = 8.3 Hz, 1H), 8.08 (d, J = 8.2 Hz, |
| | | | 1H), 7.96 (d, J = 8.3 Hz, 1H), 7.85 (t, J = 8.3 |
| | | | Hz, 1H), 7.68 (d, J = 8.0 Hz, 1H), 7.60 (t, J = |
| | | | 8.1 Hz, 1H), 7.51-7.39 (m, 5H), 5.29 (d, J = |
| | | | 10.3 Hz, 1H), 5.11-4.95 (m, 1H), 4.56-4.47 |
| | | | (m, 2H), 3.93-3.80 (m, 2H), 3.55-3.35 (m, |
| | | | 5H), 2.84 (s, 1H), 2.50 (s, 3H), 2.40-2.28 (m, |
| | | | 6H), 2.22-2.11 (m, 3H), 2.02-1.85 (m, 3H), |
| | | | 1.78 (s, 6H), 1.53 (d, J = 7.1 Hz, 3H), 1.32- |
| | | | 1.29 (m, 4H), 1.14 (d, J = 6.6 Hz, 3H), 0.75 |
| | | | (d, J = 6.2 Hz, 3H). |
| B137 | 859.3 | 859.1 | 1H NMR (400 MHz, MeOD) δ 8.94 (s, 1H), |
| | | | 8.45 (d, J = 8.4 Hz, 1H), 7.99 (d, J = 8.4 Hz, |
| | | | 2H), 7.84 (t, J = 8.3 Hz, 1H), 7.66 (d, J = 7.8 |
| | | | Hz, 1H), 7.53 (t, J = 8.0 Hz, 1H), 7.47-7.34 |
| | | | (m, 5H), 5.29 (d, J = 10.3 Hz, 1H), 5.05 (d, J = |
| | | | 6.8 Hz, 2H), 4.66-4.43 (m, 5H), 3.94-3.80 |
| | | | (m, 2H), 3.20-2.92 (m, 3H), 2.58-2.57 (m, |
| | | | 1H), 2.48 (s, 3H), 2.25-2.22 (m, 3H), 2.03- |
| | | | 1.95 (m, 3H), 1.92-1.83 (m, 2H), 1.74 (s, 6H), |
| | | | 1.54 (d, J = 7.0 Hz, 3H), 1.15 (d, J = 6.6 Hz, |
| | | | 3H), 0.78 (d, J = 6.6 Hz, 3H). |
| B161 | 859.3 | 859.6 | 1H NMR (400 MHz, MeOD) δ 10.02 (s, 1H), |
| | | | 8.70 (d, J = 8.6 Hz, 1H), 8.48 (d, J = 8.1 Hz, |
| | | | 1H), 8.23 (d, J = 8.1 Hz, 1H), 8.05 (t, J = 8.3 |
| | | | Hz, 1H), 7.88 (d, J = 8.0 Hz, 1H), 7.74-7.43 |
| | | | (m, 6H), 5.53 (d, J = 9.9 Hz, 1H), 5.13-5.02 |
| | | | (m, 2H), 4.62-4.42 (m, 2H), 3.94-3.69 (m, |
| | | | 2H), 3.15 (d, J = 10.7 Hz, 2H), 2.67-2.64 (m, |
| | | | 4H), 2.31-2.29 (m, 3H), 2.09-1.93 (m, 5H), |
| | | | 1.92-1.90 (m, 7H), 1.54 (d, J = 7.0 Hz, 3H), |
| | | | 1.19 (d, J = 6.3 Hz, 3H), 0.86 (d, J = 7.5 Hz, |
| | | | 3H). |
| B162 | 859.3 | 859.6 | 1H NMR (400 MHz, MeOD-d4) δ 8.90 (s, |
| | | | 1H), 8.45 (d, J = 8.0 Hz, 1H), 8.04-7.93 (m, |
| | | | 2H), 7.83 (t, J = 8.3 Hz, 1H), 7.65 (d, J = 8.0 |
| | | | Hz, 1H), 7.53 (t, J = 8.0 Hz, 1H), 7.48-7.38 |
| | | | (m, 5H), 5.28 (d, J = 10.4 Hz, 1H), 5.15-4.99 |
| | | | (m, 1H), 4.59-4.42 (m, 2H), 4.00-3.75 (m, |
| | | | 2H), 3.15 (t, J = 9.8 Hz, 1H), 2.95 (t, J = 11.9 |
| | | | Hz, 1H), 2.64-2.53 (m, 1H), 2.48 (s, 3H), |
| | | | 2.24-2.22 (m, 3H), 2.04-1.83 (m, 5H), 1.79- |
| | | | 1.67 (m, 8H), 1.53 (d, J = 7.0 Hz, 3H), 1.14 |
| | | | (d, J = 6.7 Hz, 3H), 0.83-0.72 (m, 3H). |

### Compound B116. (2S,4R)-1-((R)-2-(3-(2-(4-chloro-7,7-dimethyl-5-oxo-5,7-dihydroindolo[1,2-a]quinazolin-8-yl)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide

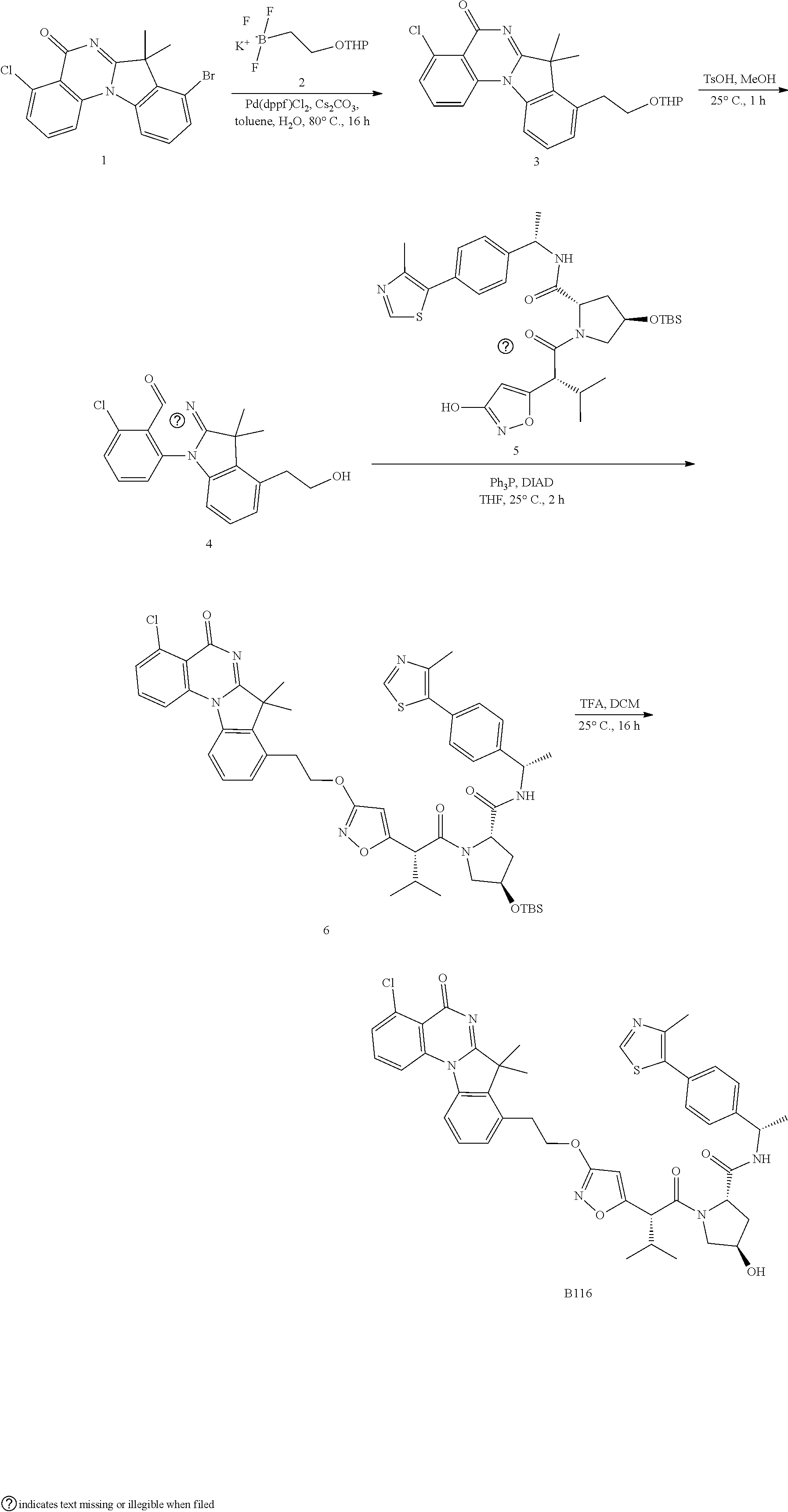

### Synthesis of 4-chloro-7,7-dimethyl-8-(2-((tetrahydro-2H-pyran-2-yl)oxy)ethyl)indolo[1,2-a]quinazolin-5 (7H)-one (Intermediate 3)

To a solution of 8-bromo-4-chloro-7,7-dimethylindolo[1,2-a]quinazolin-5 (7H)-one (300 mg, 1 eq, 0.80 mmol) in toluene (6 mL) and water (1.5 mL) were added potassium 2-(tetrahydro-2H-pyran-2-yloxy)ethyltrifluoroborate (226.2 mg, 1.2 eq, 0.96 mmol), Cs2CO3 (780.6 mg, 3 eq, 2.40 mmol) and PdCl2 (dppf) (58.44 mg, 0.1 eq, 0.08 mmol). The mixture was stirred at 80° C. for 16 h under N₂. LCMS indicated completion of reaction. The reaction was poured into water (15 mL) and extracted with EA (30 ml). The organic layer was washed with brine, dried over Na2SO4, filtered and concentrated. The crude product was purified by silica gel prep-TLC (PE:EA=2:1) to get 4-chloro-7,7-dimethyl-8-(2-((tetrahydro-2H-pyran-2-yl)oxy)ethyl)indolo[1,2-a]quinazolin-5 (7H)-one (180 mg, 0.42 mmol, 53.0%) as a white solid. LC purity (0.03% TFA): 100.00% (UV at 254 nm)/MS: 425.2 [M+H]; Retention time: 1.56 min.

### Synthesis of 4-chloro-8-(2-hydroxyethyl)-7,7-dimethylindolo[1,2-a]quinazolin-5(7H)-one (Intermediate 4)

To a solution of 4-chloro-7,7-dimethyl-8-(2-((tetrahydro-2H-pyran-2-yl)oxy)ethyl)indolo[1,2-a]quinazolin-5 (7H)-one (200 mg, 1 eq, 0.47 mmol) in methanol (3 mL) was added p-toluenesulfonicacidmonohydrate (107 mg, 0.087 mL, 1.2 eq, 0.57 mmol). The mixture was stirred at 25° C. for 2 h. LCMS indicated completion of reaction. The reaction was poured into water (15 mL) and extracted with EA (30 mL). The organic layer was washed with brine, dried over Na₂SO₄, filtered and concentrated. The crude product was purified by silica gel prep-TLC (CH₃OH/DCM=1/20) to get 4-chloro-8-(2-hydroxyethyl)-7,7-dimethylindolo[1,2-a]quinazolin-5 (7H)-one (150 mg, 0.43 mmol, 91.4%) as a yellow solid. LC purity (0.1% FA): 97.79% (UV at 254 nm)/MS: 341.2 [M+H]; Retention time: 1.40 min.

### Synthesis of (2S,4R)-4-((tert-butyldimethylsilyl)oxy)-1-((R)-2-(3-(2-(4-chloro-7,7-dimethyl-5-oxo-5,7-dihydroindolo[1,2-a]quinazolin-8-yl)ethoxy)isoxazol-5-yl)-3-methylbutanoyl)-N—((S)-1-(4-(4-methylthiazol-5-yl)phenyl)ethyl)pyrrolidine-2-carboxamide (Intermediate 6)

To a solution of 4-chloro-8-(2-hydroxyethyl)-7,7-dimethylindolo[1,2-a]quinazolin-5 (7H)-one (50 mg, 1 eq, 0.15 μmol) and (2S,4R)-4-((tert-butyldimethylsilyl)oxy)-1-((R)-2-(3-hydroxyisoxazol-5-yl)-3-methylbutanoyl)-N—((S)-1-(4-(4-methylthiazol-5-yl)phenyl)ethyl)pyrrolidine-2-carboxamide (98.9 mg, 1.1 eq, 0.16 mmol) in THF (3 mL) were added triphenylphosphine (115 mg, 0.098 mL, 3 eq, 0.44 mmol) and DIAD (59.3 mg, 0.057 mL, 2 eq, 0.29 mmol). The mixture was stirred at 25° C. for 16 h. LCMS indicated completion of reaction. The reaction was poured into water and extracted with EA. The organic layer was washed brine, dried over Na₂SO₄, filtered and concentrated. The residue was purified by silica gel chromatography (PE:EA=1:1) to get (2S,4R)-4-((tert-butyldimethylsilyl)oxy)-1-((R)-2-(3-(2-(4-chloro-7,7-dimethyl-5-oxo-5,7-dihydroindolo[1,2-a]quinazolin-8-yl)ethoxy)isoxazol-5-yl)-3-methylbutanoyl)-N—((S)-1-(4-(4-methylthiazol-5-yl)phenyl)ethyl)pyrrolidine-2-carboxamide (110.0 mg, 0.10 mmol, 68.9%, 85.97% purity) as a white solid. LC purity (0.1% NH₄HCO₃): 85.97% (UV at 254 nm)/MS: 935.5 [M+H]; Retention time: 1.97 min.

### Synthesis of (2S,4R)-1-((R)-2-(3-(2-(4-chloro-7,7-dimethyl-5-oxo-5,7-dihydroindolo[1,2-a]quinazolin-8-yl)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide (B116)

To a solution of (2S,4R)-4-((tert-butyldimethylsilyl)oxy)-1-((R)-2-(3-(2-(4-chloro-7,7-dimethyl-5-oxo-5,7-dihydroindolo[1,2-a]quinazolin-8-yl)ethoxy)isoxazol-5-yl)-3-methylbutanoyl)-N—((S)-1-(4-(4-methylthiazol-5-yl)phenyl)ethyl)pyrrolidine-2-carboxamide (110 mg, 1 eq, 0.12 mmol) in DCM (3 mL) was added TFA (13.4 mg, 0.09 mL, 10 eq, 1.2 mmol). The mixture was stirred at 25° C. for 16 h. LCMS indicated completion of reaction. The reaction was concentrated under vacuum and purified prep-HPLC (Waters 3767/Qda Column: SunFire Sunfire C18, 19*250 mm, 10 um; Mobile Phase A: 0.1% FA/H₂O, B:ACN; flow rate: 20 ml/min; gradient: 30-40% Retention Time: 8.4-9.9 min of 17 min) to afford (2S,4R)-1-((R)-2-(3-(2-(4-chloro-7,7-dimethyl-5-oxo-5,7-dihydroindolo[1,2-a]quinazolin-8-yl)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide (27.64 mg, 0.034 mmol, 28.6%) as a white solid. LC purity (0.03% TFA): 99.86% (UV at 254 nm)/MS: 821.5 [M+H]; Retention time: 1.680 min. ¹H NMR (400 MHz, DMSO) δ 8.98 (s, 1H), 8.40-8.43 (m, 2H), 8.05 (d, J=8.4 Hz, 1H), 7.86-7.77 (m, 1H), 7.64 (d, J=8.0 Hz, 1H), 7.52-7.50 (m, 1H), 7.45-7.43 (m, 2H), 7.38-7.35 (m, 3H), 6.08 (s, 1H), 4.92-4.90 (m, 1H), 4.52-4.50 (m, 2H), 4.37-4.35 (t, J=8.0 Hz, 1H), 4.30-4.28 (m, 1H), 3.72-3.62 (m, 2H), 3.37-3.35 (m, 4H), 2.45 (s, 3H), 2.25-2.23 (m, 1H), 2.10-1.98 (m, 1H), 1.79-1.77 (m, 1H), 1.63 (s, 6H), 1.42-1.40 (m, 3H), 0.98-0.95 (m, 3H), 0.78 (d, J=6.8 Hz, 3H).

The examples in the table below were prepared according to the same method as example B116 using appropriate starting materials

| | Calcd. | Found | |
|---|---|---|---|
| Ex. | (M + H)+ m/z | (M + H)+ m/z | HNMR |
| B130 | 831.3 | 831.2 | 1H NMR (400 MHz, MeOD) δ 9.08 (d, J = |
| | | | 7.6 Hz, 1H), 8.87 (s, 1H), 8.40 (d, J = 8.8 Hz, |
| | | | 1H), 8.16 (d, J = 8.4 Hz, 1H), 7.84 (t, J = 8.4 |
| | | | Hz, 1H), 7.65 (t, J = 7.6 Hz, 1H), 7.55-7.52 |
| | | | (m, 3H), 7.47-7.32 (m, 3H), 6.10 (s, 1H), |
| | | | 5.25-5.16 (m, 3H), 5.11-5.03 (m, 1H), 4.45- |
| | | | 4.44 (m, 1H), 3.91 (dd, J = 10.8, 4.4 Hz, 1H), |
| | | | 3.76-3.73 (m, 2H), 2.49 (s, 3H), 2.19 (t, J = |
| | | | 8.8 Hz, 1H), 2.00-1.95 (m, 1H), 1.60-1.55 (m, |
| | | | 5H), 1.48 (s, 3H), 1.29-1.28 (m, 1H), 1.12- |
| | | | 1.09 (m, 3H), 0.92 (d, J = 9.6 Hz, 3H). |

### Compound B139. (2S,4R)—N—((R)-2-(4-((4-(4-chloro-7,7-dimethyl-5-oxo-5,7-dihydroindolo[1,2-a]quinazolin-8-yl)piperidin-1-yl)methyl)piperidin-1-yl)-1-(4-(4-methylthiazol-5-yl)phenyl)ethyl)-4-hydroxy-1-(3-methyl-2-(3-methylisoxazol-5-yl)butanoyl)pyrrolidine-2-carboxamide

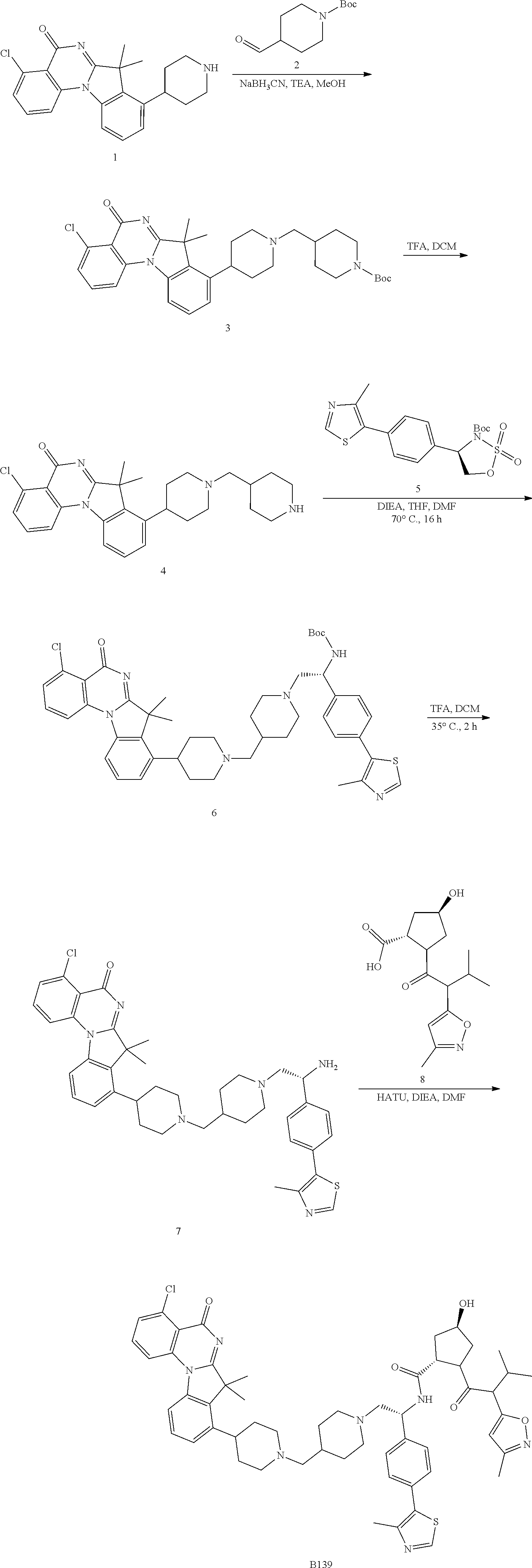

### Synthesis of tert-butyl 4-((4-(4-chloro-7,7-dimethyl-5-oxo-5,7-dihydroindolo[1,2-a]quinazolin-8-yl)piperidin-1-yl)methyl)piperidine-1-carboxylate (Intermediate 3)

To a solution of 4-chloro-7,7-dimethyl-8-(piperidin-4-yl)indolo[1,2-a]quinazolin-5 (7H)-one (300 mg, 1 eq, 790 μmol) in MeOH (4 mL) were added tert-butyl 4-formylpiperidine-1-carboxylate (253 mg, 1.5 eq, 1.18 mmol) tert-butyl 4-formylpiperidine-1-carboxylate (253 mg, 1.5 eq, 1.18 mmol), TEA (320 mg, 440 μL, 4 eq, 3.16 mmol) and sodium cyanoborohydride (99.2 mg, 91.9 μL, 2 eq, 1.58 mmol). The reaction mixture was stirred at 25° C. for 1 h. LCMS indicated completion of reaction. The mixture was poured into water (50 mL) and extracted with ethyl acetate (40 mL×3). The combined organic layers were washed with brine (50 mL), dried over sodium sulfate, filtered and concentrated under vacuum. The resulting residue was purified by silica gel chromatography (100-200 mesh silica gel, 20% of EtOAc in PE) to afford tert-butyl 4-((4-(4-chloro-7,7-dimethyl-5-oxo-5,7-dihydroindolo[1,2-a]quinazolin-8-yl)piperidin-1-yl)methyl)piperidine-1-carboxylate (250 mg, 0.26 mmol, 33%) as a yellow solid. LC purity (0.03% FA): 62.93% (UV at 254 nm)/MS: 577.1 [M+H]; Retention time: 1.40 min.

### Synthesis of tert-butyl 4-chloro-7,7-dimethyl-8-(1-(piperidin-4-ylmethyl)piperidin-4-yl)indolo[1,2-a]quinazolin-5 (7H)-one (Intermediate 4)

To a solution of tert-butyl 4-((4-(4-chloro-7,7-dimethyl-5-oxo-5,7-dihydroindolo[1,2-a]quinazolin-8-yl)piperidin-1-yl)methyl)piperidine-1-carboxylate (250 mg, 1 eq, 433 μmol) in DCM (2 mL) was added TFA (49.4 mg, 33.4 μL, 1 eq, 433 μmol) at rt. The reaction was stirred 25° C. for 2 h. LCMS showed reaction was complete. The reaction mixture was concentrated. To the residue was added DCM (20 mL) and concentrated. The above process was repeated three times to get 4-chloro-7,7-dimethyl-8-(1-(piperidin-4-ylmethyl)piperidin-4-yl)indolo[1,2-a]quinazolin-5 (7H)-one (200 mg, 0.38 mmol, 87%) as a yellow solid. LC purity (0.03% FA): 100% (UV at 254 nm)/MS: 477.1 [M+H]; Retention time: 1.91 min.

### Synthesis of tert-butyl (R)-(2-(4-((4-(4-chloro-7,7-dimethyl-5-oxo-5,7-dihydroindolo[1,2-a]quinazolin-8-yl)piperidin-1-yl)methyl)piperidin-1-yl)-1-(4-(4-methylthiazol-5-yl)phenyl)ethyl)carbamate (Intermediate 6)

To a solution of 4-chloro-7,7-dimethyl-8-(1-(piperidin-4-ylmethyl)piperidin-4-yl)indolo[1,2-a]quinazolin-5 (7H)-one (60.0 mg, 1 Eq, 126 μmol) in ACN (0.60 mL) were added DIEA (65.0 mg, 87.6μ, 4 eq, 503 μmol) and tert-butyl (R)-4-(4-(4-methylthiazol-5-yl)phenyl)-1,2,3-oxathiazolidine-3-carboxylate 2,2-dioxide (49.9 mg, 1 eq, 126 μmol). The reaction mixture was stirred at 25° C. for 2 h. LCMS indicated completion of reaction. The mixture was poured into water (50 mL) and extracted with ethyl acetate (40 mL×3). The combined organic layers were washed with brine (50 mL), dried over sodium sulfate, filtered and concentrated under vacuum. The resulting residue was purified by silica gel chromatography (100-200 mesh silica gel, 20% of EtOAc in PE) to afford tert-butyl (R)-(2-(4-((4-(4-chloro-7,7-dimethyl-5-oxo-5,7-dihydroindolo[1,2-a]quinazolin-8-yl)piperidin-1-yl)methyl)piperidin-1-yl)-1-(4-(4-methylthiazol-5-yl)phenyl)ethyl)carbamate (60.0 mg, 53 μmol, 42%, 70% purity) as a yellow solid. LC purity (0.1% FA): 80% (UV at 254 nm)/MS: 793.1 [M+H]; Retention time: 1.23 min.

### Synthesis of tert-butyl (R)-8-(1-((1-(2-amino-2-(4-(4-methylthiazol-5-yl)phenyl)ethyl)piperidin-4-yl)methyl)piperidin-4-yl)-4-chloro-7,7-dimethylindolo[1,2-a]quinazolin-5 (7H)-one (Intermediate 7)

To a solution of tert-butyl (R)-(2-(4-((4-(4-chloro-7,7-dimethyl-5-oxo-5,7-dihydroindolo[1,2-a]quinazolin-8-yl)piperidin-1-yl)methyl)piperidin-1-yl)-1-(4-(4-methylthiazol-5-yl)phenyl)ethyl)carbamate (60 mg, 1 Eq, 75.6 μmol) in DCM (1.00 mL) was added TFA (8.62 mg, 5.63 μL, 1 eq, 75.6 μmol) at rt. The reaction was stirred 25° C. for 2 h. LCMS showed reaction worked. The mixture was added DCM (1 mL) and concentrated. The above process was repeated three times to get (R)-8-(1-((1-(2-amino-2-(4-(4-methylthiazol-5-yl)phenyl)ethyl)piperidin-4-yl)methyl)piperidin-4-yl)-4-chloro-7,7-dimethylindolo[1,2-a]quinazolin-5 (7H)-one (50 mg, 58 mmol, 76%) as a yellow solid. LC purity (0.03% FA): 100% (UV at 254 nm)/MS: 693.4 [M+H]; Retention time: 1.30 min.

### Synthesis of tert-butyl (2S,4R)—N—((R)-2-(4-((4-(4-chloro-7,7-dimethyl-5-oxo-5,7-dihydroindolo[1,2-a]quinazolin-8-yl)piperidin-1-yl)methyl)piperidin-1-yl)-1-(4-(4-methylthiazol-5-yl)phenyl)ethyl)-4-hydroxy-1-(3-methyl-2-(3-methylisoxazol-5-yl)butanoyl)pyrrolidine-2-carboxamide (B139)

To a solution of (2S,4R)-4-hydroxy-1-(3-methyl-2-(3-methylisoxazol-5-yl)butanoyl)pyrrolidine-2-carboxylic acid (32.1 mg, 1.5 eq, 108 μmol) in DMF (1 mL) were added HATU (41.1 mg, 1.5 eq, 108 μmol), DIEA (28.0 mg, 37.7μ, 3 eq, 216 μmol) and (R)-8-(1-((1-(2-amino-2-(4-(4-methylthiazol-5-yl)phenyl)ethyl)piperidin-4-yl)methyl)piperidin-4-yl)-4-chloro-7,7-dimethylindolo[1,2-a]quinazolin-5 (7H)-one (50.0 mg, 1 eq, 72.1 μmol). The mixture was stirred at 25° C. for 5 h under N₂. LCMS indicated completion of reaction. The mixture was poured into water (20 mL) and extracted with ethyl acetate (20 mL×2). The combined organic layers were washed with brine (50.0 mL), dried over sodium sulfate, filtered and concentrated under vacuum. The mixture was concentrated under vacuum and the residue was purified prep-HPLC (Waters 2767/Qda, Column: SunFire Sunfire C18, 19*250 mm, 10 um; Mobile Phase A: 0.1% FA/H₂O, B:ACN; flow rate: 20 ml/min; gradient: 20%˜27%; Retention Time: 8.7-9.8 min of 17 min) to afford (2S,4R)—N—((R)-2-(4-((4-(4-chloro-7,7-dimethyl-5-oxo-5,7-dihydroindolo[1,2-a]quinazolin-8-yl)piperidin-1-yl)methyl)piperidin-1-yl)-1-(4-(4-methylthiazol-5-yl)phenyl)ethyl)-4-hydroxy-1-(3-methyl-2-(3-methylisoxazol-5-yl)butanoyl)pyrrolidine-2-carboxamide (1.98 mg, 1.82 μmol, 2.53%) as a white solid. ¹H NMR (400 MHz, MeOD) δ 8.91 (s, 1H), 8.44 (d, J=8.7 Hz, 1H), 8.03 (d, J=7.6 Hz, 1H), 7.83 (t, J=8.1 Hz, 1H), 7.67 (d, J=7.9 Hz, 1H), 7.60-7.47 (m, 5H), 7.39 (d, J=7.8 Hz, 1H), 6.27 (s, 1H), 5.43-5.41 (m, 1H), 4.52-4.50 (m, 2H), 3.92-3.80 (m, 2H), 3.74-3.63 (m, 2H), 3.47-3.38 (m, 2H), 2.95-2.93 (m, 3H), 2.80-2.78 (m, 4H), 2.49 (s, 3H), 2.30-2.28 (m, 3H), 2.10-2.06 (m, 10H), 1.74 (s, 6H), 1.34-1.31 (m, 5H), 1.13-1.10 (m, 3H), 0.88 (d, J=7.0 Hz, 3H).

The examples in the table below were prepared according to the same method as example B139 using appropriate starting materials

| | Calcd. | Found | |
|---|---|---|---|
| Ex. | (M + H)+ m/z | (M + H)+ m/z | HNMR |
| B153 | 874.3 | 874.5 | 1H NMR (400 MHz, MeOD) δ 8.89 (s, 1H), |
| | | | 8.44 (d, J = 8.5 Hz, 1H), 8.01 (d, J = 8.1 Hz, |
| | | | 1H), 7.83 (t, J = 8.3 Hz, 1H), 7.66 (d, J = 7.9 |
| | | | Hz, 1H), 7.54-7.42 (m, 6H), 6.26 (s, 1H), |
| | | | 5.24 (s, 1H), 4.64 (t, J = 7.9 Hz, 1H), 4.48 (s, |
| | | | 1H), 3.91 (d, J = 9.3 Hz, 1H), 3.73-3.71 (m, |
| | | | 2H), 3.51-3.49 (m, 1H), 3.18-3.15 (m, 4H), |
| | | | 2.83-2.64 (m, 1H), 2.51-2.50 (m, 4H), 2.27- |
| | | | 2.02 (m, 7H), 1.93-1.91 (m, 2H), 1.73 (s, 6H), |
| | | | 1.30-1.28 (m, 1H), 1.09 (d, J = 6.6 Hz, 3H), |
| | | | 0.90 (d, J = 6.7 Hz, 3H). |
| B154 | 874.3 | 874.5 | 1H NMR (400 MHz, MeOD) δ 8.89 (s, 1H), |
| | | | 8.44 (d, J = 8.5 Hz, 1H), 8.01 (d, J = 8.1 Hz, |
| | | | 1H), 7.83 (t, J = 8.3 Hz, 1H), 7.66 (d, J = 7.9 |
| | | | Hz, 1H), 7.54-7.42 (m, 6H), 6.26 (s, 1H), |
| | | | 5.26-5.24 (m, 1H), 4.64 (t, J = 7.9 Hz, 1H), |
| | | | 4.48 (s, 1H), 3.91 (d, J = 9.3 Hz, 1H), 3.73- |
| | | | 3.71 (m, 2H), 3.51-3.49 (m, 1H), 3.20-3.15 |
| | | | (m, 4H), 2.83-2.64 (m, 1H), 2.51-2.50 (m, |
| | | | 4H), 2.27-2.02 (m, 7H), 1.93-1.91 (m, 2H), |
| | | | 1.75-1.73 (m, 6H), 1.29-1.28 (m, 1H), 1.09 |
| | | | (d, J = 6.6 Hz, 3H), 0.90 (d, J = 6.7 Hz, 3H). |
| B155 | 957.4 | 957.6 | 1H NMR (400 MHz, MeOD) δ 8.88 (s, 1H), |
| | | | 8.44 (d, J = 8.6 Hz, 1H), 8.00 (d, J = 8.2 Hz, |
| | | | 1H), 7.85-7.81 (m, 1H), 7.7.67-7.65 (m, 1H), |
| | | | 7.56-7.52 (m, 1H), 7.47-7.35 (m, 5H), 6.27- |
| | | | 6.21 (m, 1H), 5.11-5.09 (m, 1H), 4.64-4.43 |
| | | | (m, 2H), 3.90-3.52 (m, 3H), 3.30-3.13 (m, |
| | | | 5H), 2.86-2.40 (m, 10H), 2.28-2.11 (m, 6H), |
| | | | 1.99-1.87 (m, 8H), 1.72 (s, 6H), 1.10-1.07 (m, |
| | | | 3H), 0.95-0.84 (m, 3H). |

### Compound B144. (2S,4R)—N—((S)-1-(4-(4-(3-(4-(4-chloro-7,7-dimethyl-5-oxo-5,7-dihydroindolo[1,2-a]quinazolin-9-yl)piperidin-1-yl) prop-1-yn-1-yl)thiazol-5-yl)phenyl)ethyl)-4-hydroxy-1-(3-methyl-2-(3-methylisorazol-5-yl)butanoyl)pyrrolidine-2-carboxamide

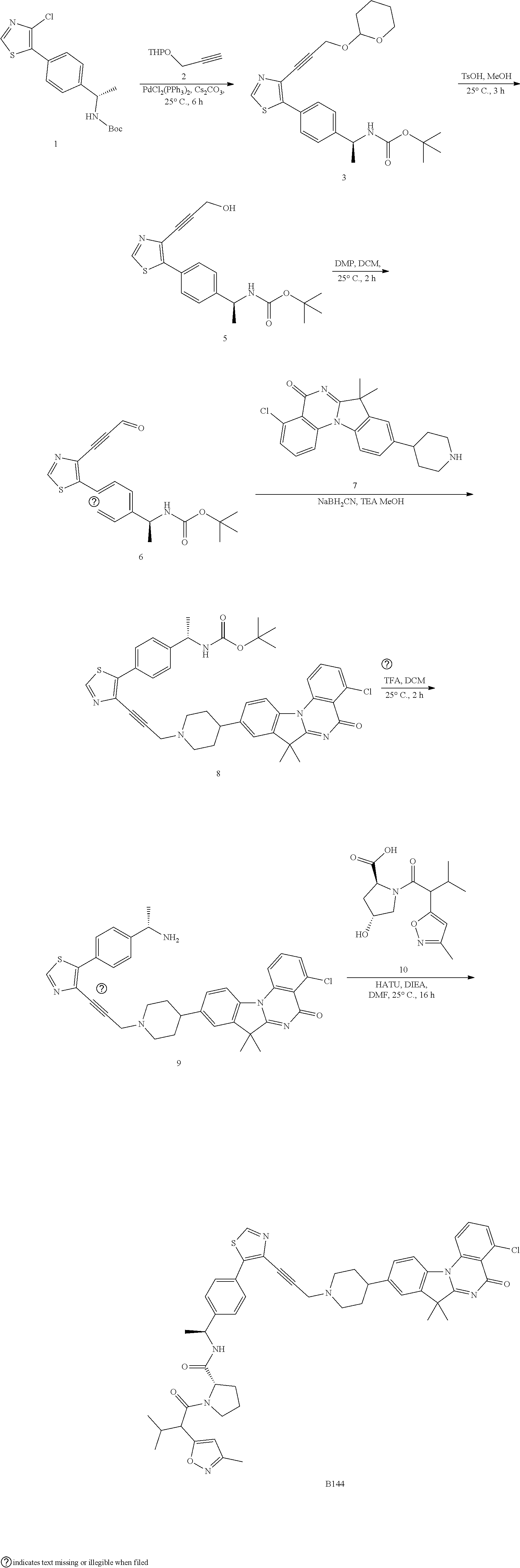

### Synthesis of tert-butyl(S)-(1-(4-(oxazol-5-yl)phenyl)ethyl)carbamate (Intermediate 3)

To a solution of tert-butyl(S)-(1-(4-(4-chlorothiazol-5-yl)phenyl)ethyl)carbamate (1 g, 1 eq, 2.95 mmol) in DMF (10 mL) were added tricyclohexylphosphoniumtetrafluoroborate (163 mg, 0.15 eq, 443 μmol), 2-(prop-2-yn-1-yloxy)tetrahydro-2H-pyran (1.24 g, 3 eq, 8.85 mmol), PdCl2 (dppf) (216 mg, 0.1 eq, 295 μmol), DBU (225 mg, 222 μL, 0.5 eq, 1.48 mmol) and Cs2CO3 (1.92 g, 2 eq, 5.90 mmol). The mixture was stirred at 145° C. for 20 min under microwave irradiation. LCMS indicated completion of reaction. To the mixture was added water (50 mL) and extracted with EA (40 mL×3). The combined organic layers were dried over sodium sulfate, filtered and concentrated under vacuum. The resulting residue was purified by prep-HPLC (FA) to afford tert-butyl ((1S)-1-(4-(4-(3-((tetrahydro-2H-pyran-2-yl)oxy)prop-1-yn-1-yl)thiazol-5-yl)phenyl)ethyl)carbamate (650 mg, 1.2 mmol, 42%) as yellow oil. LC purity (0.1% FA): 77.33% (UV at 254 nm)/MS: 443.2 [M+H]; Retention time: 1.731 min.

### Synthesis of tert-butyl(S)-(1-(4-(4-(3-hydroxyprop-1-yn-1-yl)thiazol-5-yl)phenyl)ethyl)carbamate (Intermediate 5)

To a solution of tert-butyl ((1S)-1-(4-(4-(3-((tetrahydro-2H-pyran-2-yl)oxy)prop-1-yn-1-yl)thiazol-5-yl)phenyl)ethyl)carbamate (104 mg, 1 eq, 235 μmol) in MeOH (2 mL) was added p-toluenesulfonic acid (40.5 mg, 36.4 μL, 1 eq, 235 μmol). The mixture was stirred at 25° C. for 4 h. LCMS indicated completion of reaction. The mixture was poured into saturated ammonium chloride solution (20 mL) and extracted with ethyl acetate (20 mL×2). The combined organic layers were washed with brine (50.0 mL), dried over sodium sulfate, filtered and concentrated under vacuum. The resulting residue was purified by column (PE/EA=2/1) to afford tert-butyl(S)-(1-(4-(4-(3-hydroxyprop-1-yn-1-yl)thiazol-5-yl)phenyl)ethyl)carbamate (30.0 mg, 82 μmol, 35%) as yellow solid. LC purity (0.1% FA): 74.92% (UV at 254 nm)/MS: 359.7 [M+H]; Retention time: 1.044 min.

### Synthesis of tert-butyl(S)-(1-(4-(4-(3-oxoprop-1-yn-1-yl)thiazol-5-yl)phenyl)ethyl)carbamate (Intermediate 6)

To a solution of tert-butyl(S)-(1-(4-(4-(3-hydroxyprop-1-yn-1-yl)thiazol-5-yl)phenyl)ethyl)carbamate (260 mg, 1 eq, 0.73 mmol) in DCM (4 mL) was added DMP (615.3 mg, 2 eq, 1.45 mmol) at 0° C. The mixture was stirred at 25° C. for 5 h. LCMS indicated completion of reaction. The mixture was poured into saturated ammonium chloride solution (20 mL) and extracted with ethyl acetate (20 mL×2). The combined organic layers were washed with brine (50.0 mL), dried over sodium sulfate, filtered and concentrated under vacuum. The resulting residue was purified by column chromatography (100-200 mesh silica gel, PE/EA=30/1) to afford tert-butyl(S)-(1-(4-(4-(3-oxoprop-1-yn-1-yl)thiazol-5-yl)phenyl)ethyl)carbamate (240 mg, 0.61 mmol, 84%) as yellow solid. LC purity (0.1% FA): 78.53% (UV at 254 nm)/MS: 357.1 [M+H]; Retention time: 1.171 min.

### Synthesis of tert-butyl(S)-(1-(4-(4-(3-(4-(4-chloro-7,7-dimethyl-5-oxo-5,7-dihydroindolo[1,2-a]quinazolin-9-yl)piperidin-1-yl) prop-1-yn-1-yl)thiazol-5-yl)phenyl)ethyl)carbamate (Intermediate 8)

To a solution of tert-butyl(S)-(1-(4-(4-(3-oxoprop-1-yn-1-yl)thiazol-5-yl)phenyl)ethyl)carbamate (30 mg, 1 eq, 84.2 μmol) in MeOH (2 mL) were added NaCNBH3 (10.6 mg, 2.0 eq, 168 μmol), 4-chloro-7,7-dimethyl-9-(piperidin-4-yl)indolo[1,2-a]quinazolin-5 (7H)-one (32.0 mg, 1 eq, 84.2 μmol) and TEA (25.6 mg, 35.2 μL, 3.0 eq, 252 μmol). The mixture was stirred at 25° C. for 16 h. LCMS indicated completion of reaction. The mixture was poured into saturated ammonium chloride solution (20 mL) and extracted with ethyl acetate (20 mL×2). The combined organic layers were washed with brine (50.0 mL), dried over sodium sulfate, filtered and concentrated under vacuum. The resulting residue was purified by prep-TLC (EA) to afford tert-butyl(S)-(1-(4-(4-(3-(4-(4-chloro-7,7-dimethyl-5-oxo-5,7-dihydroindolo[1,2-a]quinazolin-9-yl)piperidin-1-yl) prop-1-yn-1-yl)thiazol-5-yl)phenyl)ethyl)carbamate (20.0 mg, 14 μmol, 16%) as white solid. LC purity (0.1% FA): 80.07% (UV at 254 nm)/MS: 720.3 [M+H]; Retention time: 1.079 min.

### Synthesis of(S)-9-(1-(3-(5-(4-(1-aminoethyl)phenyl)thiazol-4-yl) prop-2-yn-1-yl)piperidin-4-yl)-4-chloro-7,7-dimethylindolo[1,2-a]quinazolin-5 (7H)-one (Intermediate 9)

A solution of tert-butyl(S)-(1-(4-(4-(3-(4-(4-chloro-7,7-dimethyl-5-oxo-5,7-dihydroindolo[1,2-a]quinazolin-8-yl)piperidin-1-yl) prop-1-yn-1-yl)thiazol-5-yl)phenyl)ethyl)carbamate (50.0 mg, 1 eq, 69.4 μmol) in DCM (1 mL) and TFA (0.50 mL) was stirred at 25° C. for 2 h. LCMS indicated completion of reaction. The mixture was concentrated under vacuum to give(S)-9-(1-(3-(5-(4-(1-aminoethyl)phenyl)thiazol-4-yl) prop-2-yn-1-yl)piperidin-4-yl)-4-chloro-7,7-dimethylindolo[1,2-a]quinazolin-5 (7H)-one (50.0 mg, 64 μmol, 93%, 80% purity) as a yellow solid. LC purity (0.03% TFA): 48.8% (UV at 254 nm)/MS: 620.1 [M+H]; Retention time: 0.959 min.

### Synthesis of (2S,4R)—N—((S)-1-(4-(4-(3-(4-(4-chloro-7,7-dimethyl-5-oxo-5,7-dihydroindolo[1,2-a]quinazolin-9-yl)piperidin-1-yl) prop-1-yn-1-yl)thiazol-5-yl)phenyl)ethyl)-4-hydroxy-1-(3-methyl-2-(3-methylisoxazol-5-yl)butanoyl)pyrrolidine-2-carboxamide (B144)

To a solution of(S)-9-(1-(3-(5-(4-(1-aminoethyl)phenyl)thiazol-4-yl) prop-2-yn-1-yl)piperidin-4-yl)-4-chloro-7,7-dimethylindolo[1,2-a]quinazolin-5 (7H)-one (25.0 mg, 1 eq, 40.3 μmol) in DMF (1 mL) were added 2-(3H-[1,2,3]triazolo[4,5-b]pyridin-3-yl)-1,1,3,3-tetramethylisouronium hexafluorophosphate (V) (30.7 mg, 2.0 eq, 80.6 μmol), N-ethyl-N-isopropylpropan-2-amine (15.6 mg, 3.0 eq, 121 μmol) and (2S,4R)-4-hydroxy-1-(3-methyl-2-(3-methylisoxazol-5-yl)butanoyl)pyrrolidine-2-carboxylic acid (11.9 mg, 1 eq, 40.3 μmol). The mixture was stirred at 25° C. for 2 h. LCMS indicated completion of reaction. The mixture was poured into saturated ammonium chloride solution (20 mL) and extracted with ethyl acetate (20 mL×2). The combined organic layers were washed with brine (50.0 mL), dried over sodium sulfate, filtered and concentrated under vacuum. The resulting residue was purified by prep-HPLC (0.1% FA in ACN) to afford (2S,4R)—N—((S)-1-(4-(4-(3-(4-(4-chloro-7,7-dimethyl-5-oxo-5,7-dihydroindolo[1,2-a]quinazolin-9-yl)piperidin-1-yl) prop-1-yn-1-yl)thiazol-5-yl)phenyl)ethyl)-4-hydroxy-1-(3-methyl-2-(3-methylisoxazol-5-yl)butanoyl)pyrrolidine-2-carboxamide (2.30 mg, 2.51 μmol, 6.23%) as white solid. LC purity (0.03% TFA): 100.0% (UV at 254 nm)/MS: 898.6 [M+H]; Retention time: 1.368 min. 1HNMR (400 MHz, MeOD-d4) δ 8.93 (s, 1H), 8.43 (d, J=8.5 Hz, 1H), 8.10-8.07 (m, 1H), 7.89-7.76 (m, 3H), 7.67-7.65 (m, 1H), 7.56 (s, 1H), 7.45-7.43 (m, 3H), 6.18-6.17 (m, 1H), 5.03-5.01 (m, 2H), 4.40-4.38 (m, 1H), 3.85-3.79 (m, 1H), 3.78-3.75 (m, 2H), 3.70-3.68 (m, 1H), 3.58-3.57 (m, 1H), 3.22-3.20 (m, 2H), 2.74-2.72 (m, 1H), 2.57-2.55 (m, 2H), 2.40-2.36 (m, 1H), 2.25-2.22 (m, 3H), 2.16-2.14 (m, 1H), 1.99-1.97 (m, 2H), 1.87-1.85 (m, 2H), 1.59 (s, 6H), 1.52-1.49 (m, 3H), 1.04-1.02 (m, 3H), 0.86-0.84 (m, 3H).

The examples in the table below were prepared according to the same method as example B144 using appropriate starting materials

| | Calcd. | Found | |
|---|---|---|---|
| Ex. | (M + H)+ m/z | (M + H)+ m/z | HNMR |
| B145 | 956.4 | 956.6 | 1H NMR (400 MHz, MeOD) δ 8.92 (d, J = |
| | | | 5.0 Hz, 1H), 8.44 (d, J = 8.7 Hz, 1H), 7.99 (d, |
| | | | J = 8.2 Hz, 1H), 7.85-7.80 (m, 3H), 7.65 (d, J = |
| | | | 7.9 Hz, 1H), 7.53-7.49 (m, 1H), 7.45-7.39 |
| | | | (m, 2H), 7.34-7.32(m, 1H), 6.21-6.17 (m, |
| | | | 1H), 5.05-4.99 (m, 1H), 4.59-4.39 (m, 4H), |
| | | | 3.85-3.58 (m, 5H), 3.16-3.08 (3H), 2.60-2.58 |
| | | | (m, 2H), 2.39-2.38 (m, 1H), 2.29-2.19 (m, |
| | | | 6H), 1.97-1.79 (m, 7H), 1.71 (s, 6H), 1.60- |
| | | | 1.48 (m, 3H), 1.06-1.04 (m, 3H), 0.93-0.80 |
| | | | (m, 3H). |
| B150 | 898.3 | 898.4 | 1H NMR (400 MHz, MeOD) δ 8.94 (s, 1H), |
| | | | 8.44 (d, J = 8.1 Hz, 1H), 8.00 (d, J = 8.2 Hz, |
| | | | 1H), 7.86-7.78 (m, 3H), 7.66 (d, J = 7.9 Hz, |
| | | | 1H), 7.56-7.52 (m, 1H), 7.46-7.36 (m, 3H), |
| | | | 6.22-6.13 (m, 1H), 5.05-4.93 (m, 1H), 4.56- |
| | | | 4.35 (m, 2H), 3.83-3.66 (m, 4H), 3.58-3.40 |
| | | | (m, 2H), 3.14-3.10 (m, 1H), 2.63-2.60 (m, |
| | | | 2H), 2.39-2.35 (m, 1H), 2.25-2.17 (m, 3H), |
| | | | 2.12-1.89 (m, 6H), 1.70 (s, 6H), 1.57-1.31 (m, |
| | | | 4H), 1.04-0.99 (m, 3H), 0.88-0.78 (m, 3H). |

### Compound B147. N-(4-(2-(4-(4-chloro-7,7-dimethyl-5-oxo-5,7-dihydroindolo[1,2-a]quinazolin-10-yl)piperidin-1-yl)ethoxy)butyl)-5-(4-((S)-1-((2S,4R)-4-hydroxy-1-((R)-3-methyl-2-(3-methylisoxazol-5-yl)butanoyl)pyrrolidine-2-carboxamido)ethyl)phenyl)thiazole-4-carboxamide

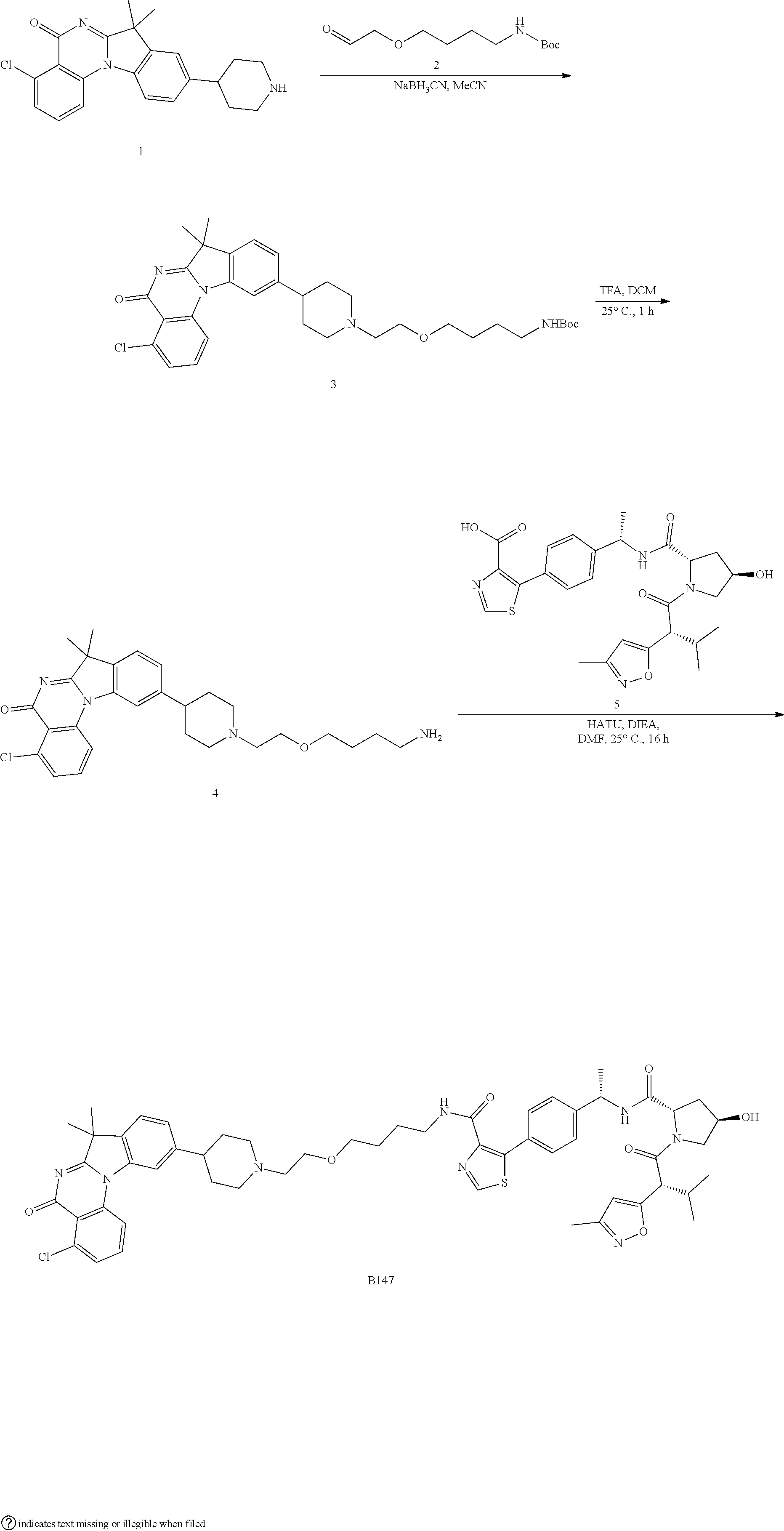

### Synthesis of tert-butyl (4-(2-(4-(4-chloro-7,7-dimethyl-5-oxo-5,7-dihydroindolo[1,2-a]quinazolin-10-yl)piperidin-1-yl)ethoxy)butyl)carbamate (Intermediate 3)

To a solution of tert-butyl (4-(2-oxoethoxy)butyl)carbamate (80.0 mg, 1 eq, 346 μmol) in MeOH (5 mL) were added 4-chloro-7,7-dimethyl-9-(piperidin-4-yl)indolo[1,2-a]quinazolin-5 (7H)-one (131 mg, 1 eq, 346 μmol), TEA (59.8 mg, 0.082 mL, 5 eq, 0.591 mmol) and sodium cyanoborohydride (32.6 mg, 30.2 μL, 1.5 eq, 519 μmol)). The mixture was stirred at 25° C. for 16 h. LCMS indicated completion of reaction. The reaction was poured into water (30 mL) and extracted with EA (30 mL×2). The organic layer was washed with brine, dried over Na₂SO₄, filtered and concentrated under vacuum. The residue was purified by silica gel column chromatography (100-200 mesh size, eluted with PE/EA=1/1) to get tert-butyl (4-(2-(4-(4-chloro-7,7-dimethyl-5-oxo-5,7-dihydroindolo[1,2-a]quinazolin-10-yl)piperidin-1-yl)ethoxy)butyl)carbamate (100 mg, 152 μmol, 43.9%) as a yellow oil. LC purity (0.1% FA): 90.68% (UV at 254 nm)/MS: 595.6 [M+H]; Retention time: 1.254 min.

### Synthesis of 10-(1-(2-(4-aminobutoxy)ethyl)piperidin-4-yl)-4-chloro-7,7-dimethylindolo[1,2-a]quinazolin-5 (7H)-one (Intermediate 4)

To a solution of tert-butyl (4-(2-(4-(4-chloro-7,7-dimethyl-5-oxo-5,7-dihydroindolo[1,2-a]quinazolin-10-yl)piperidin-1-yl)ethoxy)butyl)carbamate (100 mg, 1 eq, 168 μmol) in DCM (4 mL) was added TFA (2 mL). The mixture was stirred at 25° C. for 2 h. LCMS showed the reaction was complete. The crude product was purified by prep-HPLC (0.1FA % in CH3CN and H2O) to get 10-(1-(2-(4-aminobutoxy)ethyl)piperidin-4-yl)-4-chloro-7,7-dimethylindolo[1,2-a]quinazolin-5 (7H)-one (80 mg, 0.15 mmol, 87%) as a white solid. LC purity (0.1% FA): 90.63% (UV at 254 nm)/MS: 495.2 [M+H]; Retention time: 1.04 min.

### Synthesis of N-(4-(2-(4-(4-chloro-7,7-dimethyl-5-oxo-5,7-dihydroindolo[1,2-a]quinazolin-10-yl)piperidin-1-yl)ethoxy)butyl)-5-(4-((S)-1-((2S,4R)-4-hydroxy-1-((R)-3-methyl-2-(3-methylisoxazol-5-yl)butanoyl)pyrrolidine-2-carboxamido)ethyl)phenyl)thiazole-4-carboxamide (B147)

To a solution of 5-(4-((1S)-1-((2S,4R)-4-hydroxy-1-(3-methyl-2-(3-methylisoxazol-5-yl)butanoyl)pyrrolidine-2-carboxamido)ethyl)phenyl)thiazole-4-carboxylic acid (128 mg, 1.5 eq, 242 μmol) in DMF (5 mL) were added ((1H-benzo[d][1,2,3]triazol-1-yl)oxy)tris (dimethylamino)phosphonium hexafluorophosphate (V) (107 mg, 1.5 eq, 242 μmol), N-ethyl-N-isopropylpropan-2-amine (62.7 mg, 3 eq, 485 μmol) and ((1H-benzo[d][1,2,3]triazol-1-yl)oxy)tris (dimethylamino)phosphonium hexafluorophosphate (V) (107 mg, 1.5 eq, 242 μmol). The mixture was stirred at 25° C. for 16 h. LCMS indicated completion of reaction. The reaction was concentrated and purified by prep-HPLC, eluted with CH3CN in H2O (0.1% NH₄HCO₃) from 5.0% to 95.0% to give N-(4-(2-(4-(4-chloro-7,7-dimethyl-5-oxo-5,7-dihydroindolo[1,2-a]quinazolin-10-yl)piperidin-1-yl)ethoxy)butyl)-5-(4-((1S)-1-((2S,4R)-4-hydroxy-1-(3-methyl-2-(3-methylisoxazol-5-yl)butanoyl)pyrrolidine-2-carboxamido)ethyl)phenyl)thiazole-4-carboxamide (7.77 mg, 7.14 μmol, 4.42%) as a white solid. LC purity (0.1% FA): 92.2% (UV at 254 nm)/MS: 1003.4 [M+H]; Retention time: 1.617 min. ¹H NMR (400 MHz, MeOD) δ 8.90 (s, 1H), 8.42-8.36 (m, 1H), 8.05-7.94 (m, 1H), 7.83-7.78 (m, 1H), 7.65-7.58 (m, 1H), 7.51-7.46 (m, 4H), 7.40-7.36 (m, 1H), 7.33-7.28 (m, 2H), 6.20-6.16 (m, 1H), 4.99-4.96 (m, 1H), 4.56-4.48 (m, 1H), 4.44-4.42 (m, 1H), 3.83-3.76 (m, 1H), 3.74-3.68 (m, 1H), 3.643.58 (m, 2H), 3.49-3.42 (m, 2H), 3.36-3.30 (m, 2H), 3.22-3.09 (m, 3H), 2.70 (s, 3H), 2.27-2.25 (m, 3H), 1.87-1.85 (s, 4H), 1.68-1.66 (m, 4H), 1.59 (s, 6H), 1.48-1.42 (m, 3H), 1.31-1.26 (m, 4H), 1.05-0.96 (m, 3H), 0.90-0.80 (m, 4H).

### Compound B152. (2S,4R)—N—((S)-3-(4-(4-chloro-7,7-dimethyl-5-oxo-5,7-dihydroindolo[1,2-a]quinazolin-8-yl)piperidin-1-yl)-1-(4-(4-methylthiazol-5-yl)phenyl) propyl)-4-hydroxy-1-(3-methyl-2-(3-methylisoxazol-5-yl)butanoyl)pyrrolidine-2-carboxamide

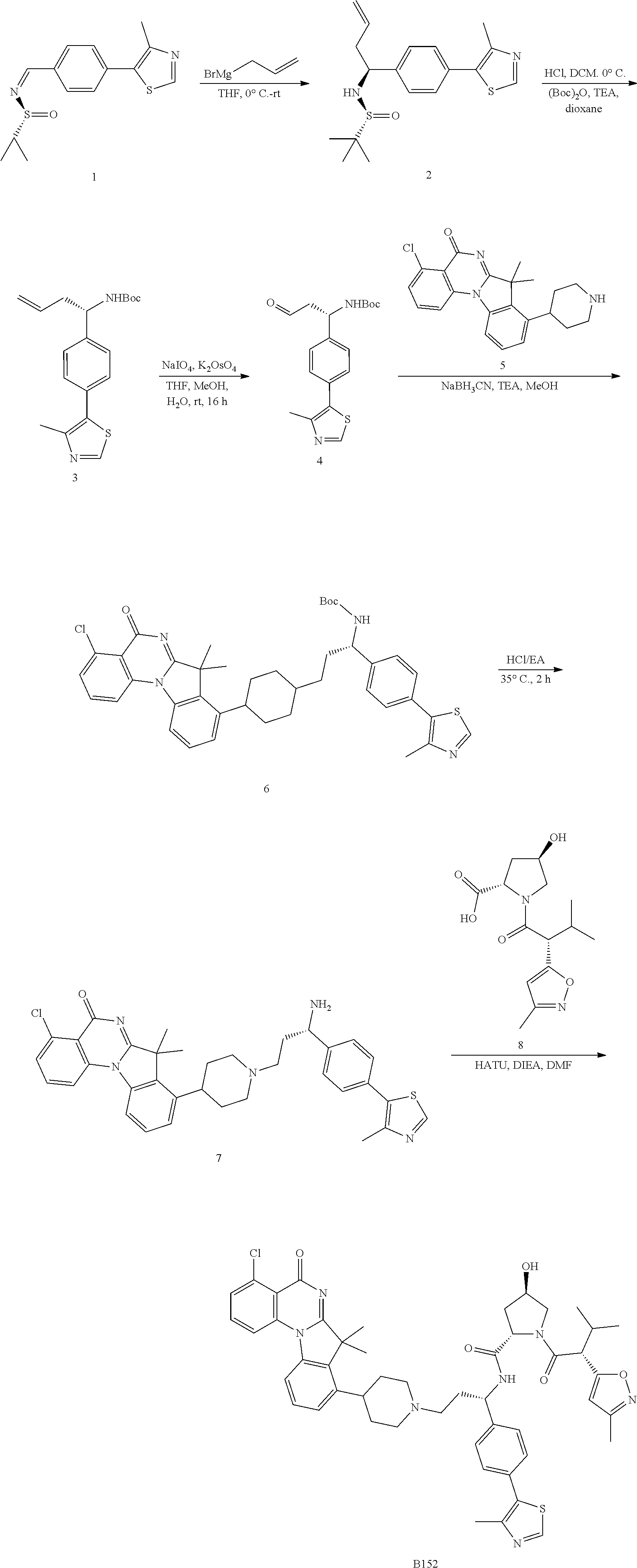

### Synthesis of 2-methyl-N—((S)-1-(4-(4-methylthiazol-5-yl)phenyl) but-3-en-1-yl) propane-2-sulfinamide (Intermediate 2)

To a mixture (Z)-2-methyl-N-(4-(4-methylthiazol-5-yl)benzylidene) propane-2-sulfinamide (15.0 g, 1 eq, 48.9 mmol) in THF (200 mL) was dropwise added allylmagnesium bromide (7.82 g, 1.1 eq, 53.8 mmol) at 0° C. for 10 h. LCMS indicated completion of reaction. The reaction mixture was quenched with water and extracted with ethyl acetate (200 mL). The organic layer was concentrated under vacuum. The residue was purified by silica gel column chromatography (100-200 mesh silica gel, 0-30% of ethyl acetate in petroleum ether) to afford 2-methyl-N—((S)-1-(4-(4-methylthiazol-5-yl)phenyl) but-3-en-1-yl) propane-2-sulfinamide (12.0 g, 32 mmol, 65%) as a colorless oil. LC purity (0.1% FA): 77.33% (UV at 254 nm)/MS: 349.1 [M+H]; Retention time: 1.198 min.

### Synthesis of tert-butyl(S)-(1-(4-(4-methylthiazol-5-yl)phenyl) but-3-en-1-yl)carbamate (Intermediate 3)

A mixture of 2-methyl-N—((S)-1-(4-(4-methylthiazol-5-yl)phenyl) but-3-en-1-yl) propane-2-sulfinamide (2.0 g, 1 eq, 2.87 mmol) in DCM/HCl (10 mL) was stirred at 20° C. for 12 h under nitrogen atmosphere. LCMS indicated completion of reaction. The mixture was filtered and concentrated under vacuum. To the crude product were added TEA (1.86 g, 3 eq, 16.9 mmol) and (Boc)₂₀ (1.47 g, 1.2 eq, 6.77 mmol). The resulting mixture was stirred at 25° C. for 2 h. LCMS indicated completion of reaction. To the reaction mixture was added water and extracted with ethyl acetate (100 mL). The organic layer was concentrated under vacuum. The residue was purified by silica gel column chromatography (100-200 mesh silica gel, 0-30% of ethyl acetate in petroleum ether) to afford tert-butyl(S)-(1-(4-(4-methylthiazol-5-yl)phenyl) but-3-en-1-yl)carbamate (1.1 g, 3.2 mmol, 80%) as a yellow oil. LC purity (0.1% TFA): 85.53% (UV at 254 nm)/MS: 345.2 [M+H]; Retention time: 1.553 min.

### Synthesis of tert-butyl(S)-(1-(4-(4-methylthiazol-5-yl)phenyl)-3-oxopropyl)carbamate (Intermediate 4)

To a solution of tert-butyl(S)-(1-(4-(4-methylthiazol-5-yl)phenyl) but-3-en-1-yl)carbamate (350 mg, 1 eq, 1.02 mmol) in THF (4 mL) and water (4 mL) were added potassiumosmate (VI) dihydrate (1.50 g, 4 eq, 4.06 mmol) and sodium metaperiodate (21.7 mg, 5.43 μL, 0.1 eq, 102 μmol). The reaction mixture was stirred at 25° C. for 3 h. LCMS indicated completion of reaction. The mixture was poured into water (50 mL) and extracted with ethyl acetate (40 mL×3). The combined organic layers were washed with brine (50 mL), dried over sodium sulfate, filtered and concentrated under vacuum. The resulting residue was purified by prep-TLC (5% of MeOH in DCM) to afford tert-butyl(S)-(1-(4-(4-methylthiazol-5-yl)phenyl)-3-oxopropyl)carbamate (160 mg, 0.32 mmol, 32%) as a yellow solid. LC purity (0.03% FA): 85.68% (UV at 254 nm)/MS: 347.1 [M+H]; Retention time: 1.348 min.

### Synthesis of tert-butyl(S)-(3-(4-(4-chloro-7,7-dimethyl-5-oxo-5,7,10,11-tetrahydroindolo[1,2-a]quinazolin-8-yl)cyclohexyl)-1-(4-(4-methylthiazol-5-yl)phenyl) propyl)carbamate (Intermediate 6)

To a mixture of tert-butyl(S)-(1-(4-(4-methylthiazol-5-yl)phenyl)-3-oxopropyl)carbamate (140 mg, 1 eq, 0.40 mmol) in MeOH (1 mL) were added TEA (50 mg, 1 eq, 0.4 mmol), 4-chloro-7,7-dimethyl-8-(piperidin-4-yl)-7,11b-dihydro-5H-indeno[2,1-c]isoquinolin-5-one (168.1 mg, 1.1 eq, 0.45 mmol) and NaBH₃CN. The mixture was stirred at 25° C. for 2 h. LCMS indicated completion of reaction. The mixture was poured into water (20 mL) and extracted with ethyl acetate (10 mL×2). The combined organic layers were washed with brine (20 mL), dried over sodium sulfate, filtered and concentrated under vacuum. The resulting residue was purified by silica gel chromatography (100-200 mesh silica gel, 3% of MeOH in DCM) to afford tert-butyl(S) (3-(4-(4-chloro-7,7-dimethyl-5-oxo-5,7,10,11-tetrahydroindolo[1,2-a]quinazolin-8-yl)cyclohexyl)-1-(4-(4-methylthiazol-5-yl)phenyl) propyl)carbamate (120 mg, 1.64 mmol, 69.6%) as a white solid. LC purity (0.1% FA): 93.31% (UV at 254 nm)/MS: 711.3 [M+H]; Retention time: 1.246 min.

### Synthesis of(S)-8-(1-(3-amino-3-(4-(4-methylthiazol-5-yl)phenyl) propyl)piperidin-4-yl)-4-chloro-7,7-dimethylindolo[1,2-a]quinazolin-5 (7H)-one (Intermediate 7)

A mixture of tert-butyl(S)-(3-(4-(4-chloro-7,7-dimethyl-5-oxo-5,7-dihydroindolo[1,2-a]quinazolin-8-yl)piperidin-1-yl)-1-(4-(4-methylthiazol-5-yl)phenyl) propyl)carbamate (40.0 mg, 1 eq, 56.3 μmol) and HCl/EA (4 mL) was stirred at 25° C. for 2 h. LCMS indicated completion of reaction. The reaction was dried over sodium sulfate, filtered and concentrated under vacuum to afford(S)-8-(1-(3-amino-3-(4-(4-methylthiazol-5-yl)phenyl) propyl)piperidin-4-yl)-4-chloro-7,7-dimethylindolo[1,2-a]quinazolin-5 (7H)-one (30.0 mg, 47 μmol, 83%) as a yellow solid. LC purity (0.1% FA): 88.62% (UV at 254 nm)/MS: 610.2 [M+1]; Retention time: 1.12 min.

### Synthesis of (2S,4R)—N—((S)-3-(4-(4-chloro-7,7-dimethyl-5-oxo-5,7-dihydroindolo[1,2-a]quinazolin-8-yl)piperidin-1-yl)-1-(4-(4-methylthiazol-5-yl)phenyl) propyl)-4-hydroxy-1-((R)-3-methyl-2-(3-methylisoxazol-5-yl)butanoyl)pyrrolidine-2-carboxamide (B152)

To a solution of (2S,4R)-4-hydroxy-1-(3-methyl-2-(3-methylisoxazol-5-yl)butanoyl)pyrrolidine-2-carboxylic acid (17.5 mg, 1.2 eq, 59.0 μmol) in DMF (2 mL) were added N-ethyl-N-isopropylpropan-2-amine (38.1 mg, 6 eq, 295 μmol), 2-(3H-[1,2,3]triazolo[4,5-b]pyridin-3-yl)-1,1,3,3-tetramethylisouronium hexafluorophosphate (V) (22.4 mg, 1.2 eq, 59.0 μmol) and(S)-8-(1-(3-amino-3-(4-(4-methylthiazol-5-yl)phenyl) propyl)piperidin-4-yl)-4-chloro-7,7-dimethylindolo[1,2-a]quinazolin-5 (7H)-one (30 mg, 1 eq, 49.2 μmol). The mixture was stirred at 25° C. for 4 h. LCMS indicated completion of reaction. The reaction was poured into H2O (30 ml) and extracted with DCM (20 ml×3), The combined organic layers were washed with brine (50 mL), dried over sodium sulfate, filtered and concentrated under vacuum. The residue was purified by prep-HPLC (Waters 3767/QdaColumn: SunFire Sunfire C18, 19*250 mm 10 um; Mobile Phase A: 0.1% FA/H₂O, B:ACN flow rate: 20 ml/min; gradient: 30-40% Retention Time: 9.4 min of 17 min)o afford (2S,4R)—N—((S)-3-(4-(4-chloro-7,7-dimethyl-5-oxo-5,7-dihydroindolo[1,2-a]quinazolin-8-yl)piperidin-1-yl)-1-(4-(4-methylthiazol-5-yl)phenyl) propyl)-4-hydroxy-1-(3-methyl-2-(3-methylisoxazol-5-yl)butanoyl)pyrrolidine-2-carboxamide (9.23 mg, 9.19 μmol, 18.7%) as a white solid. LC purity (0.1% FA): 98.91% (UV at 254 nm)/MS: 889.5 [M+H]; Retention time: 1.38 min. ¹H NMR (400 MHz, MeOD) δ 8.89 (s, 1H), 8.44 (d, J=8.7 Hz, 1H), 8.07-7.97 (m, 1H), 7.84 (t, J=8.1 Hz, 1H), 7.66 (d, J=7.8 Hz, 1H), 7.55-7.46 (m, 4H), 7.39 (d, J=7.9 Hz, 1H), 6.30 (s, 1H), 5.10-5.09 (m, 1H), 4.60-4.58 (m, 2H), 4.50-4.48 (m, 1H), 3.88-3.86 (m, 1H), 3.82-3.78 (m, 2H), 3.42-3.40 (m, 1H), 3.20-3.17 (m, 1H), 2.50-2.46 (m, 4H), 2.31-2.14 (m, 7H), 2.11-1.87 (m, 6H), 1.74 (s, 6H), 1.35-1.32 (m, 3H), 1.10-1.08 (m, 3H), 0.93-0.81 (m, 3H).

The examples in the table below were prepared according to the same method as example B152 using appropriate starting materials

| | Calcd. | Found | |
|---|---|---|---|
| Ex. | (M + H)+ m/z | (M + H)+ m/z | HNMR |
| B165 | 888.4 | 444.8 | 1H NMR (400 MHz, MeOD-d4) δ 8.89 (s, |
| | | | 1H), 8.45 (d, J = 8.6 Hz, 1H), 8.02 (d, J = 8.3 |
| | | | Hz, 1H), 7.84 (t, J = 8.3 Hz, 1H), 7.66 (d, J = |
| | | | 8.0 Hz, 1H), 7.55 (t, J = 8.1 Hz, 1H), 7.50- |
| | | | 7.45 (m, 4H), 7.39 (d, J = 8.1 Hz, 1H), 6.25 |
| | | | (s, 1H), 5.08-5.06 (m, 1H), 4.63-4.55 (m, 2H), |
| | | | 4.48 (s, 1H), 3.89 (d, J = 9.4 Hz, 1H), 3.76 (d, |
| | | | J = 11.2 Hz, 1H), 3.68 (d, J = 11.1 Hz, 1H), |
| | | | 3.50-3.48 (m, 1H), 3.15-3.13 (m, 1H), 2.50- |
| | | | 2.49 (m, 4H), 2.28-2.26 (m, 1H), 2.22 (s, 6H), |
| | | | 2.10-1.84 (m, 6H), 1.74 (s, 6H), 1.31-1.29 |
| | | | (m, 3H), 1.07 (d, J = 6.6 Hz, 3H), 0.88 (d, J = |
| | | | 6.7 Hz, 3H). |
| B166 | 888.4 | 888.4 | 1H NMR (400 MHz, MeOD-d4) δ 8.89 (s, |
| | | | 1H), 8.44 (d, J = 8.3 Hz, 1H), 8.02-8.00 (m, |
| | | | 1H), 7.83 (t, J = 8.3 Hz, 1H), 7.66 (d, J = 7.9 |
| | | | Hz, 1H), 7.55-7.45 (m, 5H), 7.39 (d, J = 7.9 |
| | | | Hz, 1H), 6.22 (d, J = 4.9 Hz, 1H), 5.10-5.08 |
| | | | (m, 1H), 4.60-4.45 (m, 2H), 3.87-3.48 (m, |
| | | | 4H), 3.00-2.51 (m, 4H), 2.50 (s, 3H), 2.30- |
| | | | 2.27 (m, 1H), 2.24-2.10 (m, 7H), 2.03-1.96 |
| | | | (m, 6H), 1.75-1.71 (m, 6H), 1.35-1.26 (m, |
| | | | 3H), 1.08 (d, J = 6.5 Hz, 3H), 0.92-0.87 (m, |
| | | | 4H). |

### Compound B159. (2S,4R)-1-((R)-2-(3-(((1r,4R)-4-((4-(4′-bromo-5′-oxo-5′H-spiro[cyclohexane-1,7′-indolo[1,2-a]quinazolin]-10′-yl)piperidin-1-yl)methyl)cyclohexyl)oxy)isorazol-5-yl)-3-methylbutanoyl)-4-hydroxy-N—((R)-1-(4-(4-methylthiazol-5-yl)phenyl)-2-morpholinoethyl)pyrrolidine-2-carboxamide

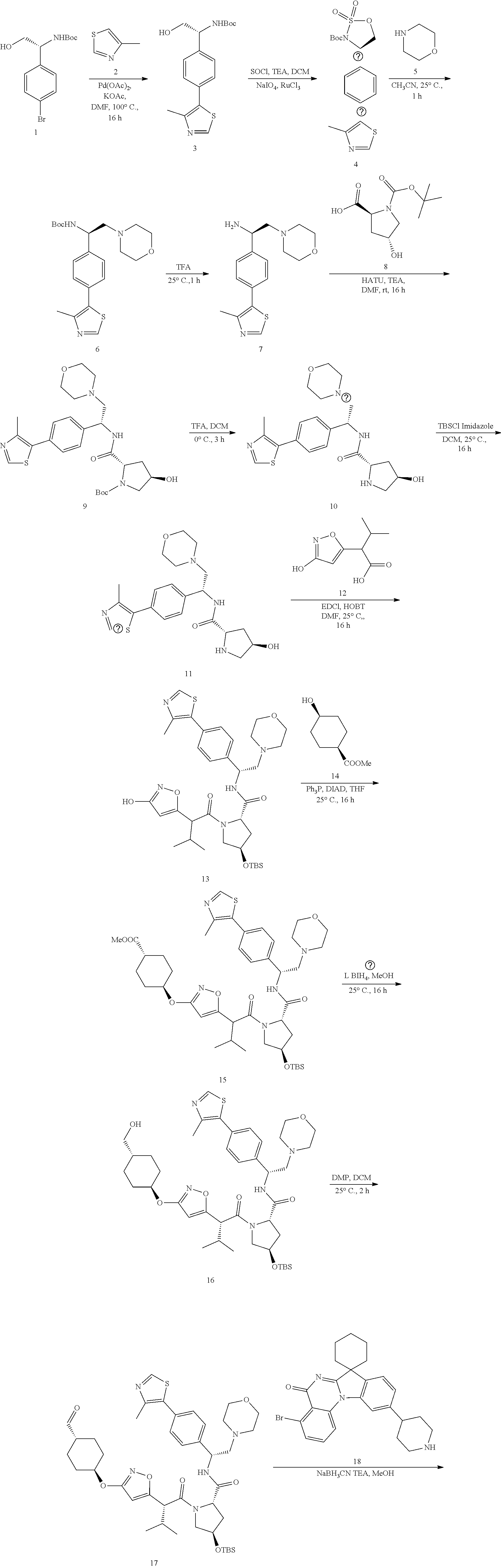

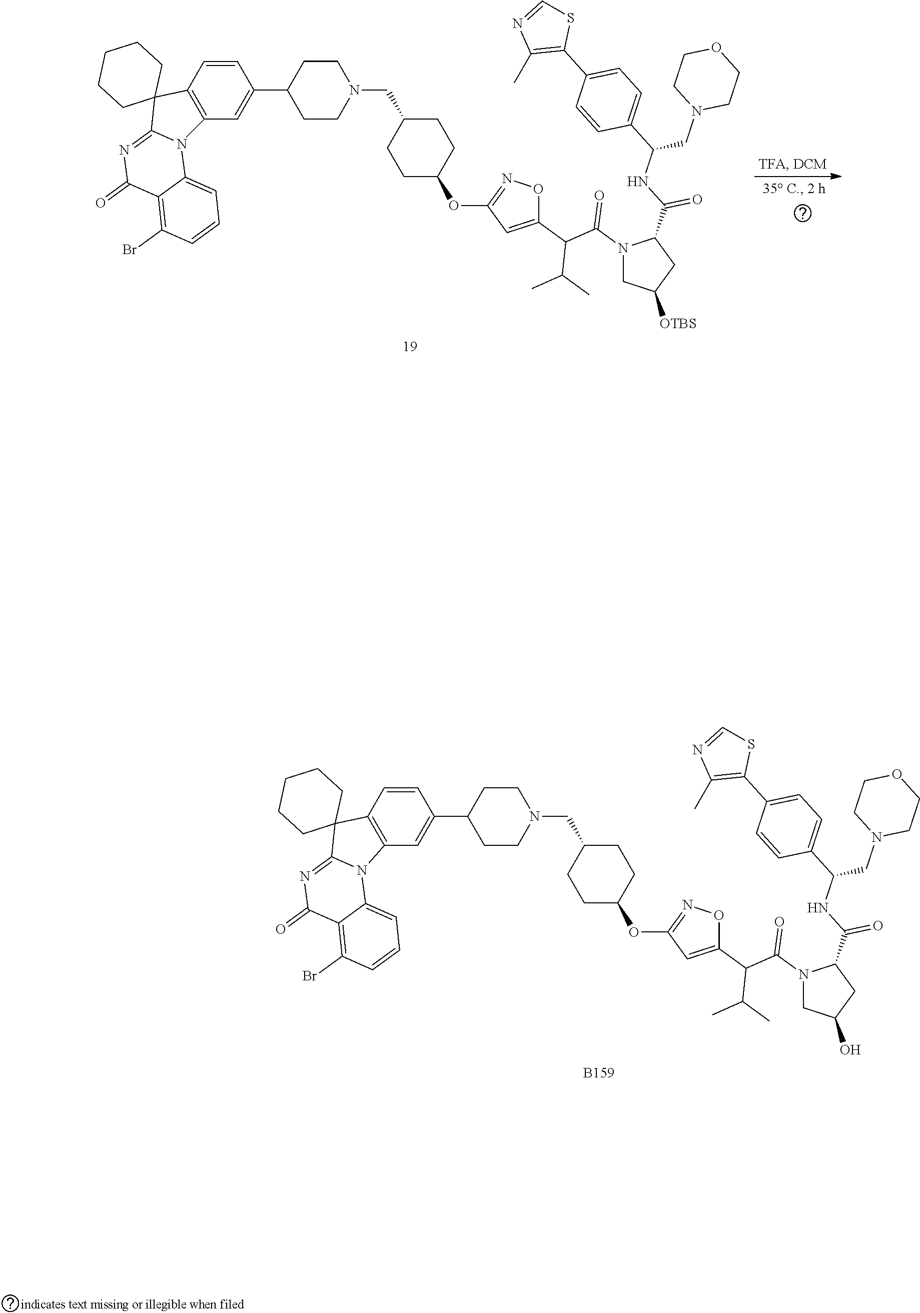

### Synthesis of (R)-(2-hydroxy-1-(4-(4-methylthiazol-5-yl)phenyl)ethyl)carbamate (Intermediate 3)

A mixture of tert-butyl (R)-(1-(4-bromophenyl)-2-hydroxyethyl)carbamate (20.0 g, 1 eq, 63.3 mmol), 4-methylthiazole (12.5 g, 2 eq, 127 mmol), potassium acetate (12.4 g, 7.91 mL, 2 eq, 127 mmol) and Pd (OAc) 2 (1.42 g, 0.1 eq, 6.33 mmol) in DMF (400 mL) was degassed and purged with nitrogen for 3 times. The mixture was stirred at 100° C. for 12 h under nitrogen atmosphere. LCMS indicated completion of reaction. The mixture was poured into water (200 mL) and extracted with ethyl acetate (200 mL×3). The combined organic layers were washed with brine (500 mL), dried over sodium sulfate, filtered and concentrated under vacuum. The residue was purified by silica gel chromatography (100-200 mesh silica gel, 80% of EA in PE) to afford tert-butyl (R)-(2-hydroxy-1-(4-(4-methylthiazol-5-yl)phenyl)ethyl)carbamate (16.0 g, 43 mmol, 68%, 90% purity) as a yellow solid. LC purity (0.1% FA): 79% (UV at 254 nm)/MS: 335.2 [M+H]; Retention time: 1.44 min.

### Synthesis of tert-butyl (R)-4-(4-(4-methylthiazol-5-yl)phenyl)-1,2,3-oxathiazolidine-3-carboxylate 2,2-dioxide (Intermediate 4)

A solution of tert-butyl (R)-(2-hydroxy-1-(4-(4-methylthiazol-5-yl)phenyl)ethyl)carbamate (8.00 g, 1 eq, 23.9 mmol) in DCM (400 mL) was added dropwise to a stirred solution of SOCl2 (3.13 g, 1.92 mL, 1.1 eq, 26.3 mmol), TEA (5.33 g, 7.34 mL, 2.2 eq, 52.6 mmol) and imidazole (6.51 g, 4 eq, 95.7 mmol) in DCM (400 mL) at −78° C. The resulting mixture was stirred at −78° C. for 3 h. The reaction mixture was warmed to room temperature overnight. The reaction mixture was quenched by addition of water and the phases were separated. The organic phase was washed with brine, dried (Na2SO4) and concentrated in vacuo to give the crude cyclic sulfamidite. To the crude cyclic sulfamidite was added ACN (100 mL) at rt. To the resulting solution was added sodium metaperiodate (8.19 g, 2.03 mL, 1.6 eq, 38.3 mmol), ruthenium (III) chloride hydrate (53.9 mg, 0.01 eq, 239 μmol) and water (100 mL). The resulting mixture was stirred until the reaction was complete by TLC. The reaction mixture was diluted with water (equal volume to that used in reaction) and extracted with Et2O. The organic layer was washed with brine, dried (Na2SO4) and concentrated in vacuo. The residue was purified by silica gel chromatography (100-200 mesh silica gel, 60% of EA in PE) to afford tert-butyl (R)-4-(4-(4-methylthiazol-5-yl)phenyl)-1,2,3-oxathiazolidine-3-carboxylate 2,2-dioxide (6.00 g, 15 mmol, 63%, 99% purity) as a yellow solid. LC purity (0.1% FA): 90% (UV at 254 nm)/MS: 397.0 [M+H]; Retention time: 1.49 min.

### Synthesis of tert-butyl (R)-(1-(4-(4-methylthiazol-5-yl)phenyl)-2-morpholinoethyl)carbamate (Intermediate 6)

To a solution of tert-butyl (R)-4-(4-(4-methylthiazol-5-yl)phenyl)-1,2,3-oxathiazolidine-3-carboxylate 2,2-dioxide (12.0 g, 1 eq, 30.3 mmol) in ACN (20 mL) was added morpholine (15.8 g, 6 eq, 182 mmol). The mixture was stirred at 25° C. for 1 h under N₂. LCMS indicated completion of reaction. The mixture was poured into water (20 mL) and extracted with ethyl acetate (20 mL×2). The combined organic layers were washed with brine (50 mL), dried over sodium sulfate, filtered and concentrated under vacuum. The mixture was concentrated under vacuum and the residue was purified by C18 column chromatography, eluted with CH₃CN in H₂O from 10% to 100% (0.1% FA) to afford tert-butyl (R)-(1-(4-(4-methylthiazol-5-yl)phenyl)-2-morpholinoethyl)carbamate (6.90 g, 15 mmol, 51%) as a white solid. LC purity (0.1% FA): 96% (UV at 254 nm)/MS: 404.2 [M+H]; Retention time: 1.0 min.

### Synthesis of (R)-1-(4-(4-methylthiazol-5-yl)phenyl)-2-morpholinoethan-1-amine (Intermediate 7)

To a solution of tert-butyl (R)-(1-(4-(4-methylthiazol-5-yl)phenyl)-2-morpholinoethyl)carbamate (6.40 g, 1 eq, 15.9 mmol) in DCM (30 mL) was added TFA (29.6 g, 20.0 mL, 16.4 eq, 260 mmol) and the reaction was stirred at 25° C. for 16 h. LCMS indicated completion of reaction. The mixture was concentrated under vacuum to afford (R)-1-(4-(4-methylthiazol-5-yl)phenyl)-2-morpholinoethan-1-amine (4.80 g, 14 mmol, 90%) as a yellow oil. LC purity (0.1% FA): 99% (UV at 254 nm)/MS: 304.4 [M+H]; Retention time: 0.2 min.

### Synthesis of tert-butyl (2S,4R)-4-hydroxy-2-(((R)-1-(4-(4-methylthiazol-5-yl)phenyl)-2-morpholinoethyl) carbamoyl)pyrrolidine-1-carboxylate (Intermediate 9)

To a solution of (R)-1-(4-(4-methylthiazol-5-yl)phenyl)-2-morpholinoethan-1-amine (4.88 g, 1.2 eq, 16.1 mmol) in DMF (100 mL) were added HATU (6.12 g, 1.2 eq, 16.1 mmol), DIEA (8.66 g, 11.7 mL, 5 eq, 67.0 mmol) and (2S,4R)-1-(tert-butoxycarbonyl)-4-hydroxypyrrolidine-2-carboxylic acid (3.10 g, 1. eq, 13.4 mmol). The reaction mixture was stirred at 25° C. for 3 h. LCMS indicated completion of reaction. The mixture was poured into water (200 mL) and extracted with ethyl acetate (200 mL×3). The combined organic layers were washed with brine (200 mL), dried over sodium sulfate, filtered and concentrated under vacuum. The resulting residue was purified by silica gel chromatography (100-200 mesh silica gel, 6% of MeOH in DCM) to afford tert-butyl (2S,4R)-4-hydroxy-2-(((R)-1-(4-(4-methylthiazol-5-yl)phenyl)-2-morpholinoethyl) carbamoyl)pyrrolidine-1-carboxylate (7.0 g, 12 mmol, 91%) as a yellow solid. LC purity (0.1% FA): 90% (UV at 254 nm)/MS: 517.2 [M+H]; Retention time: 1.28 min.

### Synthesis of (2S,4R)-4-hydroxy-N—((R)-1-(4-(4-methylthiazol-5-yl)phenyl)-2-morpholinoethyl)pyrrolidine-2-carboxamide (Intermediate 10)

A mixture of tert-butyl (2S,4R)-4-hydroxy-2-(((R)-1-(4-(4-methylthiazol-5-yl)phenyl)-2-morpholinoethyl) carbamoyl)pyrrolidine-1-carboxylate (7.00 g, 1 eq, 13.5 mmol) in DCM (10 mL) and TFA (2.50 mL) was stirred at 0° C. for 2 h. LCMS indicated completion of reaction. The mixture was concentrated under vacuum to afford (2S,4R)-4-hydroxy-N—((R)-1-(4-(4-methylthiazol-5-yl)phenyl)-2-morpholinoethyl)pyrrolidine-2-carboxamide (5.60 g, 13 mmol, 98%, 99% purity) as a yellow solid. LC purity (0.1% FA): 80% (UV at 254 nm)/MS: 417.2 [M+H]; Retention time: 0.23 min.

### Synthesis of (2S,4R)-4-((tert-butyldimethylsilyl)oxy)-N—((R)-1-(4-(4-methylthiazol-5-yl)phenyl)-2-morpholinoethyl)pyrrolidine-2-carboxamide (Intermediate 11)

To a solution of (2S,4R)-4-hydroxy-N—((R)-1-(4-(4-methylthiazol-5-yl)phenyl)-2-morpholinoethyl)pyrrolidine-2-carboxamide (5.60 g, 1 eq, 13.4 mmol) in DCM (40 mL) were added imidazole (7.32 g, 8 eq, 108 mmol and TBS-Cl (6.08 g, 3 eq, 40.3 mmol). The mixture was stirred at 25° C. for 1 h under N₂. LCMS indicated completion of reaction. The mixture was poured into water (200 mL) and extracted with ethyl acetate (200 mL×3). The combined organic layers were washed with brine (500 mL), dried over sodium sulfate, filtered and concentrated under vacuum. The residue was purified by C18 column chromatography with CH₃CN in H2O from 10% to 95%, (0.1% FA) to afford (2S,4R)-4-((tert-butyldimethylsilyl)oxy)-N—((R)-1-(4-(4-methylthiazol-5-yl)phenyl)-2-morpholinoethyl)pyrrolidine-2-carboxamide (5.40 g, 9.2 mmol, 68%, 90% purity) as a yellow solid. LC purity (0.1% FA): 90% (UV at 254 nm)/MS: 531. 2 [M+H]; Retention time: 1.27 min.

### Synthesis of (2S,4R)-4-((tert-butyldimethylsilyl)oxy)-1-(2-(3-hydroxyisoxazol-5-yl)-3-methylbutanoyl)-N—((R)-1-(4-(4-methylthiazol-5-yl)phenyl)-2-morpholinoethyl)pyrrolidine-2-carboxamide (Intermediate 13)

To a solution of (R)-2-(3-hydroxyisoxazol-5-yl)-3-methylbutanoic acid (1.36 g, 1.3 eq, 7.35 mmol) in DMF (5 mL) were added DIEA (4.38 g, 5.91 mL, 6 eq, 33.9 mmol), EDCl (1.63 g, 1.5 eq, 8.48 mmol) and HOBt (1.30 g, 1.5 eq, 8.48 mmol). The mixture was stirred at 25° C. for 10 min, and then (2S,4R)-4-((tert-butyldimethylsilyl)oxy)-N—((R)-1-(4-(4-methylthiazol-5-yl)phenyl)-2-morpholinoethyl)pyrrolidine-2-carboxamide (3.00 g, 1 eq, 5.65 mmol) was added. The mixture and stirred at 25° C. for 1 h. LCMS showed the reaction was complete. The mixture was poured into water (200 mL) and extracted with ethyl acetate (200 mL×3). The combined organic layers were washed with brine (200 mL), dried over sodium sulfate, filtered and concentrated under vacuum. The residue was purified by C18 column chromatography (10 mmol/L NH4CO3) to afford (2S,4R)-4-((tert-butyldimethylsilyl)oxy)-1-((R)-2-(3-hydroxyisoxazol-5-yl)-3-methylbutanoyl)-N—((R)-1-(4-(4-methylthiazol-5-yl)phenyl)-2-morpholinoethyl)pyrrolidine-2-carboxamide (2.00 g, 2.6 mmol, 46%, 90% purity) as a white solid. LC purity (0.1% FA): 99% (UV at 254 nm)/MS: 698.2 [M+H]; Retention time: 1.48 min.

### Synthesis of methyl (1S,4r)-4-((5-(1-((2S,4R)-4-((tert-butyldimethylsilyl)oxy)-2-(((R)-1-(4-(4-methylthiazol-5-yl)phenyl)-2-morpholinoethyl) carbamoyl) pyrrolidin-1-yl)-3-methyl-1-oxobutan-2-yl)isoxazol-3-yl)oxy)cyclohexane-1-carboxylate (Intermediate 15)

A mixture of (2S,4R)-4-((tert-butyldimethylsilyl)oxy)-1-(2-(3-hydroxyisoxazol-5-yl)-3-methylbutanoyl)-N—((R)-1-(4-(4-methylthiazol-5-yl)phenyl)-2-morpholinoethyl)pyrrolidine-2-carboxamide (2.00 g, 1 eq, 2.87 mmol), methyl (1s,4s)-4-hydroxycyclohexane-1-carboxylate (1.81 g, 4 eq, 11.5 mmol) and triphenylphosphine (2.25 g, 1.90 mL, 3 eq, 8.60 mmol) was degassed under vacuum and purged with Ar2 several times. To the mixture was added THF (5.00 mL). The resulting mixture was cooled to 0° C. in and ice water bath. To the resulting mixture was added DIAD (1.74 g, 1.67 mL, 3 eq, 8.60 mmol) and stirred at 25° C. for 16 h. LCMS showed the reaction was complete. The mixture was poured into water (200 mL) and extracted with ethyl acetate (200 mL×3). The combined organic layers were washed with brine (200 mL), dried over sodium sulfate, filtered and concentrated under vacuum. The residue was purified by silica gel chromatography (100-200 mesh silica gel, 6% of MeOH in DCM) to afford methyl (1S,4r)-4-((5-(1-((2S,4R)-4-((tert-butyldimethylsilyl)oxy)-2-(((R)-1-(4-(4-methylthiazol-5-yl)phenyl)-2-morpholinoethyl) carbamoyl) pyrrolidin-1-yl)-3-methyl-1-oxobutan-2-yl)isoxazol-3-yl)oxy)cyclohexane-1-carboxylate (1.90 g, 2.2 mmol, 75%, 95% purity) as a white solid. LC purity (0.1% FA): 90% (UV at 254 nm)/MS: 839.4 [M+H]; Retention time: 10.2 min.

### Synthesis of (2S,4R)-4-((tert-butyldimethylsilyl)oxy)-1-((R)-2-(3-(((1r,4R)-4-(hydroxymethyl)cyclohexyl)oxy)isoxazol-5-yl)-3-methylbutanoyl)-N—((R)-1-(4-(4-methylthiazol-5-yl)phenyl)-2-morpholinoethyl)pyrrolidine-2-carboxamide (Intermediate 16)

To a solution of methyl (1R,4r)-4-((5-((R)-1-((2S,4R)-4-((tert-butyldimethylsilyl)oxy)-2-(((R)-1-(4-(4-methylthiazol-5-yl)phenyl)-2-morpholinoethyl) carbamoyl) pyrrolidin-1-yl)-3-methyl-1-oxobutan-2-yl)isoxazol-3-yl)oxy)cyclohexane-1-carboxylate (950 mg, 1 eq, 1.13 mmol) in MeOH (10 mL) was added LiBH4 (987 mg, 40 eq, 45.3 mmol). The reaction mixture was stirred at 25° C. for 5 h. LCMS indicated completion of reaction. The mixture was poured into water (200 mL) and extracted with ethyl acetate (200 mL×3). The combined organic layers were washed with brine (500 mL), dried over sodium sulfate, filtered and concentrated under vacuum. The residue was purified by silica gel chromatography (100-200 mesh silica gel, 8% of MeOH in DCM) to afford (2S,4R)-4-((tert-butyldimethylsilyl)oxy)-1-((R)-2-(3-(((1r,4R)-4-(hydroxymethyl)cyclohexyl)oxy)isoxazol-5-yl)-3-methylbutanoyl)-N—((R)-1-(4-(4-methylthiazol-5-yl)phenyl)-2-morpholinoethyl)pyrrolidine-2-carboxamide (0.90 g, 1.0 mmol, 88%, 90% purity) as a yellow solid. LC purity (0.1% FA): 90% (UV at 254 nm)/MS: 810.2 [M+H]; Retention time: 1.43 min.

### Synthesis of (2S,4R)-4-((tert-butyldimethylsilyl)oxy)-1-((R)-2-(3-(((1r,4R)-4-formylcyclohexyl)oxy)isoxazol-5-yl)-3-methylbutanoyl)-N—((R)-1-(4-(4-methylthiazol-5-yl)phenyl)-2-morpholinoethyl)pyrrolidine-2-carboxamide (Intermediate 17)

To a mixture of (2S,4R)-4-((tert-butyldimethylsilyl)oxy)-1-((R)-2-(3-(((1r,4R)-4-(hydroxymethyl)cyclohexyl)oxy)isoxazol-5-yl)-3-methylbutanoyl)-N—((R)-1-(4-(4-methylthiazol-5-yl)phenyl)-2-morpholinoethyl)pyrrolidine-2-carboxamide (900 mg, 1 eq, 1.11 mmol) in DCM (10 mL) was added DMP (565 mg, 1.2 eq, 1.33 mmol). The reaction mixture was stirred at 25° C. for 0.5 h. LCMS indicated completion of reaction. The mixture was poured into water (20 mL) and extracted with ethyl acetate (20 mL×3). The combined organic layers were washed with brine (50 mL), dried over sodium sulfate, filtered and concentrated under vacuum. The residue was purified by prep-TLC (10% of MeOH in DCM) to afford (2S,4R)-4-((tert-butyldimethylsilyl)oxy)-1-((R)-2-(3-(((1r,4R)-4-formylcyclohexyl)oxy)isoxazol-5-yl)-3-methylbutanoyl)-N—((R)-1-(4-(4-methylthiazol-5-yl)phenyl)-2-morpholinoethyl)pyrrolidine-2-carboxamide (450 mg, 0.50 mmol, 45%) as a yellow solid. LC purity (0.1% FA): 92.71% (UV at 254 nm)/MS: 808.4 [M+H]; Retention time: 1.32 min.

### Synthesis of (2S,4R)-1-((R)-2-(3-(((1r,4R)-4-((4-(4′-bromo-5′-oxo-5′H-spiro[cyclohexane-1,7′-indolo[1,2-a]quinazolin]-10′-yl)piperidin-1-yl)methyl)cyclohexyl)oxy)isoxazol-5-yl)-3-methylbutanoyl)-4-((tert-butyldimethylsilyl)oxy)-N—((R)-1-(4-(4-methylthiazol-5-yl)phenyl)-2-morpholinoethyl)pyrrolidine-2-carboxamide (Intermediate 19)

To a solution of 4′-bromo-10′-(piperidin-4-yl)-5′H-spiro[cyclohexane-1,7′-indolo[1,2-a]quinazolin]-5′-one (50.0 mg, 1 eq, 108 μmol) in DCM (2 mL) were added TEA (32.7 mg, 3 eq, 0.323 mmo), AcOH (25.9 mg, 4 eq, 0.431 mmol) and sodium triacetoxyborohydride (68.5 mg, 3 eq, 0.323 mmol). The mixture and stirred at 25° C. for 1 h under N₂. LCMS indicated completion of reaction. The mixture was poured into water (20 mL) and extracted with ethyl acetate (20 mL×3). The combined organic layers were washed with brine (50 mL), dried over sodium sulfate, filtered and concentrated under vacuum. The residue was purified by HPLC to obtain (2S,4R)-1-((R)-2-(3-(((1r,4R)-4-((4-(4′-bromo-5′-oxo-5′H-spiro[cyclohexane-1,7′-indolo[1,2-a]quinazolin]-10′-yl)piperidin-1-yl)methyl)cyclohexyl)oxy)isoxazol-5-yl)-3-methylbutanoyl)-4-((tert-butyldimethylsilyl)oxy)-N—((R)-1-(4-(4-methylthiazol-5-yl)phenyl)-2-morpholinoethyl)pyrrolidine-2-carboxamide (60.0 mg, 43 μmol, 40%, 90% purity) as a yellow solid. LC purity (0.1% FA): 96.68% (UV at 254 nm)/MS: 628.8 [½M+H]; Retention time: 1.25 min

### Synthesis of (2S,4R)-1-((R)-2-(3-(((1r,4R)-4-((4-(4′-bromo-5′-oxo-5′H-spiro[cyclohexane-1,7′-indolo[1,2-a]quinazolin]-10′-yl)piperidin-1-yl)methyl)cyclohexyl)oxy)isoxazol-5-yl)-3-methylbutanoyl)-4-hydroxy-N—((R)-1-(4-(4-methylthiazol-5-yl)phenyl)-2-morpholinoethyl)pyrrolidine-2-carboxamide (B159)

A mixture of (2S,4R)-1-((R)-2-(3-(((1r,4R)-4-((4-(4′-bromo-5′-oxo-5′H-spiro[cyclohexane-1,7′-indolo[1,2-a]quinazolin]-10′-yl)piperidin-1-yl)methyl)cyclohexyl)oxy)isoxazol-5-yl)-3-methylbutanoyl)-4-((tert-butyldimethylsilyl)oxy)-N—((R)-1-(4-(4-methylthiazol-5-yl)phenyl)-2-morpholinoethyl)pyrrolidine-2-carboxamide (70.0 mg, 1 eq, 55.7 μmol) in DCM (1 mL) and TFA (3 mL) was stirred at 25° C. for 1 h under N₂. LCMS indicated completion of reaction. The mixture was concentrated under vacuum and the residue was purified by prep-HPLC (Waters 3767/Qda, Column: SunFire Sunfire C18, 19*250 mm, 10 um; Mobile Phase A: 0.1% FA/H₂O, B:ACN; flow rate: 20 ml/min; gradient: 22-32%; Retention Time: 8.4-9.9 min of 17 min) to afford (2S,4R)-1-((R)-2-(3-(((1r,4R)-4-((4-(4′-bromo-5′-oxo-5′H-spiro[cyclohexane-1,7′-indolo[1,2-a]quinazolin]-10′-yl)piperidin-1-yl)methyl)cyclohexyl)oxy)isoxazol-5-yl)-3-methylbutanoyl)-4-hydroxy-N—((R)-1-(4-(4-methylthiazol-5-yl)phenyl)-2-morpholinoethyl)pyrrolidine-2-carboxamide (20.62 mg, 16.92 μmol, 30.4%) as a white solid. LC purity (0.1% FA): 96.68% (UV at 254 nm)/MS: 572.4 [½M+H]; Retention time: 1.315 min. ¹H NMR (400 MHz, MeOD-d4) δ 8.91 (s, 1H), 8.44 (d, J=8.0 Hz, 1H), 7.98 (s, 1H), 7.92-7.80 (m, 2H), 7.73 (t, J=8.2 Hz, 1H), 7.59-7.38 (m, 4H), 7.34 (d, J=8.0 Hz, 1H), 6.01 (s, 1H), 5.60-5.55 (m, 1H), 4.57-4.42 (m, 3H), 4.01-3.98 (m, 4H), 3.91-3.88 (m, 1H), 3.81-3.68 (m, 4H), 3.52-3.33 (m, 4H), 3.25-3.04 (m, 6H), 2.48 (s, 3H), 2.43-2.37 (m, 1H), 2.36-1.92 (m, 16H), 1.90-1.66 (m, 6H), 1.62-1.49 (m, 2H), 1.36-1.21 (m, 2H), 1.08 (d, J=6.5 Hz, 3H), 0.95-0.92 (m, 3H).

The examples in the table below were prepared according to the same method as example B159 using appropriate starting materials

| | Calcd. | Found | |
|---|---|---|---|
| Ex. | (M + H)+ m/z | (M + H)+ m/z | HNMR |
| B138 | 1098.5 | 550.0 | 1H NMR (400 MHz, MeOD) δ 8.88 (s, |
| | | | 1H), 8.37 (d, J = 8.6 Hz, 1H), 7.88-7.71 |
| | | | (m, 2H), 7.64 (d, J = 7.6 Hz, 2H), 7.50- |
| | | | 7.35 (m, 4H), 7.01 (d, J = 8.1 Hz, 1H), |
| | | | 6.08-5.90 (m, 1H), 5.25-5.13 (m, 1H), |
| | | | 4.56-4.45 (m, 2H), 3.87-3.84 (m, 1H), |
| | | | 3.78 (t, J = 4.5 Hz, 3H), 3.74-3.66 (m, |
| | | | 2H), 3.65-3.63 (m, 1H), 3.55-3.39 (m, |
| | | | 4H), 3.20-2.97 (m, 4H), 2.95-2.86 (m, |
| | | | 1H), 2.86-2.61 (m, 7H), 2.58-2.52 (m, |
| | | | 1H), 2.44-2.33 (m, 1H), 2.28-2.17 (m, |
| | | | 3H), 2.62-2.12 (m, 14H), 1.61-1.39 (m, |
| | | | 2H), 1.27-1.11 (m, 2H), 1.09-1.04 (m, |
| | | | 3H), 0.97-0.89 (m, 3H). |
| B157 | 1057.5 | 1057.5 | 1H NMR (400 MHz, MeOD) δ 8.89 (s, |
| | | | 1H), 8.42 (d, J = 8.4 Hz, 1H), 7.99 (s, |
| | | | 1H), 7.86 (t, J = 8.3 Hz, 1H), 7.65 (dd, |
| | | | J = 16.7, 7.9 Hz, 2H), 7.50-7.34 (m, |
| | | | 5H), 6.00-5.97 (m, 1H), 5.24 (dd, J = |
| | | | 9.4, 5.3 Hz, 1H), 4.55-4.35 (m, 3H), |
| | | | 3.87-3.62 (m, 9H), 3.15-3.12 (m, 5H), |
| | | | 3.02-2.69 (m, 6H), 2.48 (s, 3H), 2.37- |
| | | | 2.07 (m, 8H), 2.04-1.93 (m, 4H), 1.62- |
| | | | 1.50 (m, 8H), 1.30-1.27 (m, 2H), 1.10- |
| | | | 1.07 (m, 3H), 0.95-0.93 (m, 3H). |
| B158 | 1097.5 | 1097.5 | 1H NMR (400 MHz, MeOD) δ 8.87 (s, |
| | | | 1H), 8.42 (d, J = 8.6 Hz, 1H), 7.96 (s, |
| | | | 1H), 7.86-7.84 (m, 2H), 7.65 (d, J = 7.9 |
| | | | Hz, 1H), 7.48-7.40 (m, 4H), 7.39-7.28 |
| | | | (m, 2H), 6.01-5.87 (m, 1H), 5.49 (s, |
| | | | 3H), 5.10-5.09 (m, 1H), 4.66-4.53 (m, |
| | | | 7H), 4.43-4.41 (m, 2H), 3.84-3.82 (m, |
| | | | 1H), 3.78-3.60 (m, 6H), 3.57-3.44 (m, |
| | | | 1H), 3.21-3.03 (m, 3H), 2.80-2.77 (m, |
| | | | 3H), 2.67-2.54 (m, 3H), 2.48 (s, 3H), |
| | | | 2.33-2.23 (m, 4H), 1.97-1.94 (m, 9H), |
| | | | 1.77-1.67 (m, 3H), 1.52-1.41 (m, 2H), |
| | | | 1.29-1.01 (m, 6H), 0.94-0.92 (m, 3H). |
| B163 | 1058.5 | 1058.1 | 1H NMR (400 MHz, MeOD) δ 8.88 (s, |
| | | | 1H), 8.39 (d, J = 8.5 Hz, 1H), 7.85 (t, J = |
| | | | 8.3 Hz, 1H), 7.65 (d, J = 8.2 Hz, 2H), |
| | | | 7.56-7.34 (m, 5H), 7.03 (d, J = 8.3 Hz, |
| | | | 1H), 6.02-5.89 (m, 1H), 5.18 (dd, J = |
| | | | 9.2, 5.4 Hz, 1H), 4.57-4.36 (m, 3H), |
| | | | 3.66-3.40 (m, 5H), 3.14-2.98 (m, 4H), |
| | | | 2.93-2.75 (m, 3H), 2.74-2.50 (m, 6H), |
| | | | 2.48 (s, 3H), 2.42-2.35 (m, 1H), 2.32- |
| | | | 2.13 (m, 3H), 2.00-1.98 (m, 3H), 1.81- |
| | | | 1.79 (m, 1H), 1.55 (s, 6H), 1.20-1.18 |
| | | | (m, 2H), 1.10-1.06 (m, 3H), 0.95-0.91 |
| | | | (m, 3H). |

### Compound B169. (2S,4R)-1-((S-2-(4-(3-(4-(4′-chloro-5′-oxo-5′H-spiro[cyclohexane-1,7′-indolo[1,2-a]quinazolin]-10′-yl)piperidine-1-carbonyl) bicycle[1.1.1]pentan-1-yl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide

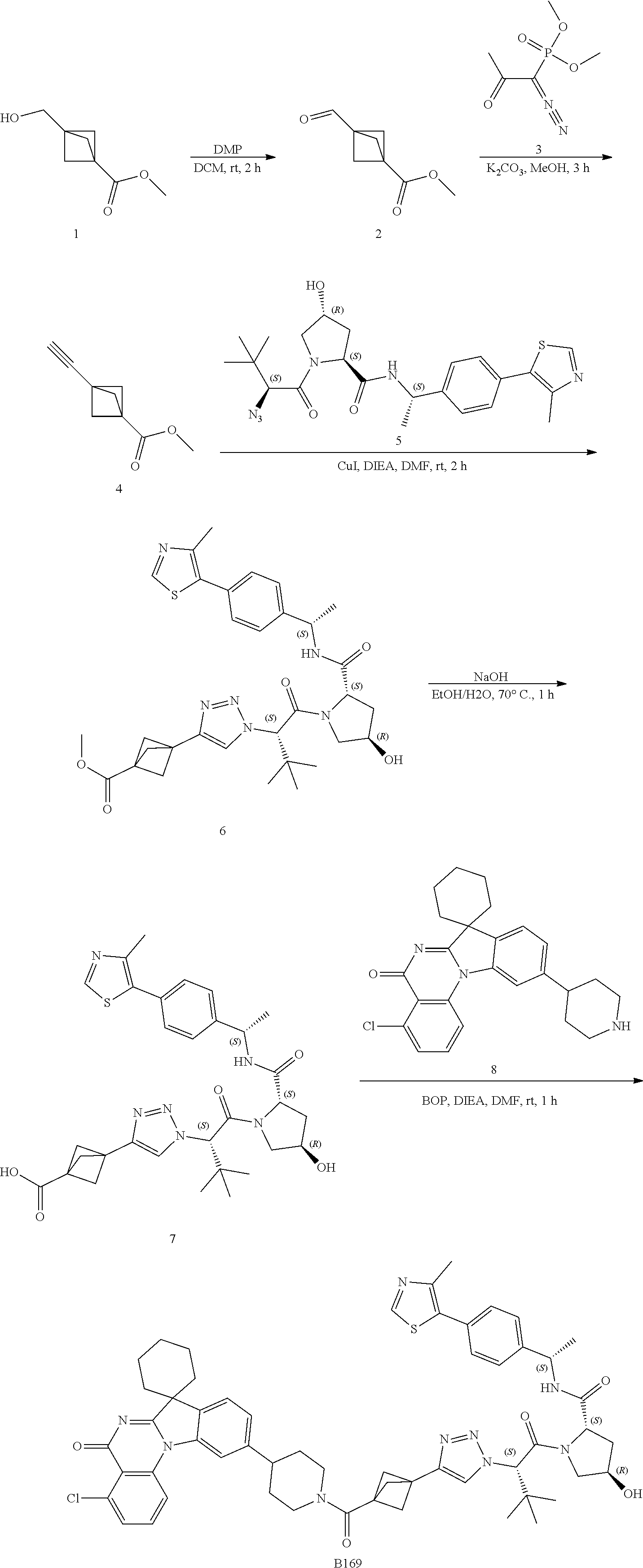

### Synthesis of methyl 3-formylbicyclo[1.1.1]pentane-1-carboxylate (Intermediate 2)

A solution of methyl 3-(hydroxymethyl)bicyclo[1.1.1]pentane-1-carboxylate (300 mg, 1.0 eq, 1.92 mmol) and Dess-Martin periodinane (2.44 g, 3.0 eq, 5.76 mmol) in DCM (10.0 mL) was stirred at 25° C. for 1.0 h in N2. Thin-layer chromatography (TLC) (petroleum ether/ethyl acetate (PE/EtOAc)=10/1, Retention factor (Rf)=0.45, DMP) showed no starting material. The reaction mixture was filtered and extracted with DCM (2×100 mL). The organic layer washed with saturated NH₄Cl (100 mL×3) and saturated NaCl (100 mL×1). The resulting organic layer was dried over anhydrous Na2SO4 and the solvent was removed in vacuo to give methyl 3-formylbicyclo[1.1.1]pentane-1-carboxylate (220 mg, 1.43 mmol, 74.3%) as a yellow oil and no need further purified.

### Synthesis of methyl 3-ethynylbicyclo[1.1.1]pentane-1-carboxylate (Intermediate 4)

To a solution of dimethyl (1-diazo-2-oxopropyl)phosphonate (274 mg, 1.0 eq, 1.43 mmol) and methyl 3-formylbicyclo[1.1.1]pentane-1-carboxylate (220 mg, 1.0 eq, 1.43 mmol) in MeOH (10.0 mL) was added potassium carbonate (197 mg, 1.0 eq, 1.43 mmol). The reaction mixture was stirred at 25° C. for 16 h under N2. Thin-layer chromatography (TLC) (petroleum ether/ethyl acetate (PE/EtOAc)=6/1, Retention factor (Rf)=0.60, DMP and Phosphomolybdic Acid) showed no starting material. The reaction was filtered and extracted with DCM (50 mL×2). The organic layer washed with saturated NH₄Cl (50 mL×3) and saturated NaCl (100 mL×1). The resulting organic layer was dried over anhydrous Na₂SO₄ and the solvent was removed in vacuo to give methyl 3-ethynylbicyclo[1.1.1]pentane-1-carboxylate (120 mg, 799 μmol, 56.0%) as a white solid.

### Synthesis of methyl 3-(1-((S)-1-((2S,4R)-4-hydroxy-2-(((S)-1-(4-(4-methylthiazol-5-yl)phenyl)ethyl) carbamoyl) pyrrolidin-1-yl)-3,3-dimethyl-1-oxobutan-2-yl)-1H-1,2,3-triazol-4-yl)bicyclo[1.1.1]pentane-1-carboxylate (Intermediate 6)

To a solution of (2S,4R)-1-((S)-2-azido-3,3-dimethylbutanoyl)-4-hydroxy-N—((S)-1-(4-(4-methylthiazol-5-yl)phenyl)ethyl)pyrrolidine-2-carboxamide (263 mg, 0.7 eq, 559 μmol) and methyl 3-ethynylbicyclo[1.1.1]pentane-1-carboxylate (120 mg, 1.0 eq, 799 μmol) in DMF (5.00 mL), diisopropylethylamine (309 mg, 0.42 mL, 3.0 eq, 799 μmol) and cuprous iodide (457 mg, 3.0 eq, 2.40 mmol) were added. The reaction mixture was stirred at 25° C. for 1 h under N₂. The reaction was monitored by LCMS. The reaction was purified by prep-TLC (DCM/MeOH)=30/1, Retention factor (Rf)=0.50) to obtain methyl 3-(1-((S)-1-((2S,4R)-4-hydroxy-2-(((S)-1-(4-(4-methylthiazol-5-yl)phenyl)ethyl) carbamoyl) pyrrolidin-1-yl)-3,3-dimethyl-1-oxobutan-2-yl)-1H-1,2,3-triazol-4-yl)bicyclo[1.1.1]pentane-1-carboxylate (90.0 mg, 145 μmol, 18.1%) as a white solid. LC purity (0.1% FA): 57.58% (UV at 254 nm)/MS: 621.2 [M+H]. Retention time: 1.57 min.

### Synthesis of 3-(1-((S)-1-((2S,4R)-4-hydroxy-2-(((S)-1-(4-(4-methylthiazol-5-yl)phenyl)ethyl) carbamoyl) pyrrolidin-1-yl)-3,3-dimethyl-1-oxobutan-2-yl)-1H-1,2,3-triazol-4-yl)bicyclo[1.1.1]pentane-1-carboxylic acid (Intermediate 7)

A mixture of methyl 3-(1-((S)-1-((2S,4R)-4-hydroxy-2-(((S)-1-(4-(4-methylthiazol-5-yl)phenyl)ethyl) carbamoyl) pyrrolidin-1-yl)-3,3-dimethyl-1-oxobutan-2-yl)-1H-1,2,3-triazol-4-yl)bicyclo[1.1.1]pentane-1-carboxylate (90.0 mg, 1.0 eq, 145 μmol) and sodium hydroxide (17.4 mg, 3.0 eq, 435 μmol) in ethanol (5 mL) and H₂O (2.0 mL) was stirred at 70° C. for 1 h inder N2. The reaction was monitored by LCMS. To the reaction mixture was added with 1.0 M HCl solution (20 mL) till the pH was 5-6. The resulting mixture was extracted with EtOAc (80 mL). The organic layer was concentrated. The residue was purified by prep-HPLC eluted with eluted with MeCN in H₂O (neutrally condition) to obtain 3-(1-((S)-1-((2S,4R)-4-hydroxy-2-(((S)-1-(4-(4-methylthiazol-5-yl)phenyl)ethyl) carbamoyl) pyrrolidin-1-yl)-3,3-dimethyl-1-oxobutan-2-yl)-1H-1,2,3-triazol-4-yl)bicyclo[1.1.1]pentane-1-carboxylic acid (70.0 mg, 115 μmol, 79.6%) as a white solid. LC purity (0.1% NH₄HCO₃): 56.5% (UV at 254 nm)/MS: 607.2 [M+H]. Retention time: 1.172 min.

### Synthesis of (2S,4R)-1-((S)-2-(4-(3-(4-(4′-chloro-5′-oxo-5′H-spiro[cyclohexane-1,7′-indolo[1,2-a]quinazolin]-10′-yl)piperidine-1-carbonyl)bicyclo[1.1.1]pentan-1-yl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide (B169)

To a solution of 4′-chloro-10′-(piperidin-4-yl)-5′H-spiro[cyclohexane-1,7′-indolo[1,2-a]quinazolin]-5′-one (48.4 mg, 1.0 eq, 115 μmol) and 3-(1-((S)-1-((2S,4R)-4-hydroxy-2-(((S)-1-(4-(4-methylthiazol-5-yl)phenyl)ethyl) carbamoyl) pyrrolidin-1-yl)-3,3-dimethyl-1-oxobutan-2-yl)-1H-1,2,3-triazol-4-yl)bicyclo[1.1.1]pentane-1-carboxylic acid (70.0 mg, 1.0 eq, 115 μmol) in DMF (5.0 mL), diisopropylethylamine (44.7 mg, 3.0 eq, 345 μmol) and benzotriazol-1-yloxy)tris (dimethylamino)phosphonium hexafluorophosphate (51.0 mg, 1.0 eq, 115.4 μmol) were added. The reaction mixture was stirred at 25° C. for 16 h under N₂. The reaction was monitored by LCMS. The reaction mixture was filtered and purified by prep-HPLC eluting with MeCN in H2O (neutral condition) to obtain (2S,4R)-1-((S)-2-(4-(3-(4-(4′-chloro-5′-oxo-5′H-spiro[cyclohexane-1,7′-indolo[1,2-a]quinazolin]-10′-yl)piperidine-1-carbonyl)bicyclo[1.1.1]pentan-1-yl)-1H-1,2,3-triazol-1-yl)-3,3-dimethylbutanoyl)-4-hydroxy-N—((S)-1-(4-(4-methylthiazol-5-yl)phenyl)ethyl)pyrrolidine-2-carboxamide (10.15 mg, 10.0 μmol, 8.66%) as a off-white solid. LC purity (0.1% FA): 98.84% (UV at 254 nm)/MS: 1008.4 [M+H]. Retention time: 1.663 min. ¹H NMR (400 MHZ, CD3OD) δ 8.94 (s, 1H), 8.46-8.43 (m, 1H), 8.17-8.15 (m, 1H), 8.08-7.98 (m, 1H), 7.88-7.84 (m, 2H), 7.77-7.65 (m, 1H), 7.50-7.32 (m, 5H), 5.53-5.52 (m, 1H), 5.04-4.91 (m, 2H), 4.54-4.28 (m, 3H), 4.03-3.47 (m, 4H), 3.13-2.68 (m, 2H), 2.57-2.47 (m, 10H), 1.54-1.53 (m, 2H), 1.07 (s, 9H).

### II. Biological Activity

### For “A” Series Compounds

### HiBiT Assay for Protein Degradation:

Hela cells were genetically modified via CRISPR/Cas9 to fuse HiBiT to the carboxy terminus of SMARCA2 (Promega CS302365) or SMARCA4 (Promega CS3023225). Cells were cultured in DMEM containing 10% FBS and 1% (Vol:Vol) penicillin-streptomycin. At the time of experiment, cells were seeded at a density of 20,000 cells per well in a 96-well plate (Corning Cat. #3903) and treated with serial dilutions of testing compounds for 24 hours. At the end of experiment, levels of SMARCA2 or SMARCA4 expression were assessed with Nano-Glo® HiBiT Lytic Detection Assay (Promega N3050). IC50 was obtained using the GraphPad Prism data analysis software. The results of the HiBiT Assay are summarized in Tables E1 and E2 below.

TABLE E1

| | SMARC A2 | | SMARC A4 | |
|---|---|---|---|---|
| | Hela HiBit | | Hela HiBit | |
| Compound No. | DC₅₀ (nM) | Dmax (%) | DC50(nM) | Dmax(%) |
| A1 | A | A | B | B |
| A2 | A | A | A | B |
| A3 | A | A | A | A |
| A4 | A | A | A | A |
| A5 | A | A | A | A |
| A6 | B | B | C | C |
| A7 | B | B | No fit | D |
| A8 | B | B | D | D |
| A9 | A | A | C | C |
| A10 | C | C | No fit | D |
| A11 | A | B | No fit | D |
| A12 | B | B | C | C |
| A13 | A | B | No fit | D |
| A14 | A | A | B | C |
| A15 | A | A | No Fit | D |
| A16 | A | A | C | C |
| A17 | A | B | A | A |
| A18 | A | B | A | B |
| A19 | A | A | A | B |
| A20 | A | A | D | D |
| A21 | B | A | B | C |
| A22 | A | A | A | A |
| A23 | A | A | B | B |
| A24 | A | A | B | B |
| A25 | D | D | D | D |
| A26 | D | D | D | D |
| A27 | A | B | C | C |
| A28 | A | B | B | C |
| A29 | A | B | D | D |
| A30 | A | B | D | D |
| A31 | D | D | D | C |
| A32 | D | D | D | B |
| A33 | A | A | B | B |
| A34 | C | C | D | D |
| A35 | A | A | A | A |
| A36 | A | A | A | A |
| A37 | A | A | A | A |
| A38 | A | A | B | B |
| A39 | A | A | A | A |
| A40 | A | A | A | A |
| A41 | A | B | No Fit | D |
| A42 | A | B | No Fit | D |
| A43 | A | A | A | B |
| A44 | A | A | A | B |
| A45 | A | A | A | B |
| A46 | A | A | A | B |
| A47 | A | A | A | A |
| A48 | A | A | B | C |
| A49 | A | A | B | B |
| A50 | A | A | No Fit | D |
| A51 | C | C | No Fit | D |
| A52 | A | B | No Fit | D |
| A53 | A | A | A | A |
| A54 | A | A | A | A |
| A55 | A | A | A | A |
| A56 | A | A | A | A |
| A57 | A | A | A | A |
| A58 | A | A | A | A |
| A59 | A | B | D | D |
| A60 | A | A | A | A |
| A61 | A | A | A | A |
| A62 | A | A | A | B |
| A63 | A | A | A | B |
| A64 | A | A | B | B |
| A65 | A | A | A | A |
| A66 | D | C | D | D |
| A67 | A | A | A | B |
| A68 | A | A | D | D |
| A69 | A | A | A | B |
| A70 | A | A | B | C |
| A71 | A | A | D | C |
| A72 | A | A | D | D |
| A73 | A | A | D | C |
| A74 | A | A | D | C |
| A75 | A | A | D | D |

Note:
DC₅₀: A (<100 nM), B (100-500 nM), C (501-1000 nM), and D (>1000 nM).
Dₘₐₓ: A (>90% degradation), B (70-90% degradation), C (50-69% degradation), and D (<50% degradation).

TABLE E2

| | SMARC A2 | | SMARC A4 | |
|---|---|---|---|---|
| | Hela HiBit | | Hela HiBit | |
| Compound No. | DC₅₀ (nM) | Dmax (%) | | DC₅₀ (nM) |
| A76 | A | A | D | D |
| A77 | B | B | D | D |
| A78 | B | B | D | D |
| A79 | A | B | D | C |
| A80 | B | B | D | C |
| A81 | A | A | D | C |
| A82 | A | A | D | C |
| A83 | A | A | D | C |
| A84 | B | A | D | D |
| A85 | A | A | B | C |
| A86 | A | A | D | C |
| A87 | A | A | D | C |
| A88 | B | A | B | B |
| A89 | A | A | D | C |
| A90 | A | A | D | D |
| A91 | A | A | D | D |
| A92 | A | B | D | D |
| A93 | A | A | D | D |
| A94 | A | A | D | D |
| A95 | A | A | D | D |
| A96 | A | A | D | C |
| A97 | A | A | D | C |
| A98 | A | A | D | D |
| A99 | A | A | D | D |
| A100 | A | A | D | C |
| A101 | A | A | B | B |
| A102 | A | A | D | C |
| A103 | A | A | A | C |
| A104 | A | A | D | C |
| A105 | C | C | D | D |
| A106 | C | B | C | D |
| A107 | C | B | D | D |
| A108 | A | B | D | D |
| A109 | C | B | D | C |
| A110 | B | A | D | C |
| A111 | A | A | B | B |
| A112 | A | A | C | C |
| A113 | A | A | No fit | B |
| A114 | B | B | C | B |
| A115 | B | B | B | A |
| A116 | D | D | D | D |
| A117 | D | D | D | D |
| A118 | A | A | C | C |
| A119 | A | A | B | B |
| A120 | A | A | No fit | C |
| A121 | A | A | No fit | B |
| A122 | A | A | No fit | C |
| A123 | A | A | B | B |
| A124 | A | A | No fit | D |
| A125 | A | A | No fit | C |
| A126 | A | A | B | C |
| A127 | A | A | A | B |
| A128 | C | C | D | D |
| A129 | C | C | D | D |
| A130 | B | A | D | D |
| A131 | B | B | D | D |
| A132 | A | A | D | D |
| A133 | C | B | D | D |
| A134 | C | B | D | D |
| A135 | A | A | A | A |
| A136 | A | A | A | A |
| A137 | A | A | A | B |
| A138 | A | A | A | A |
| A139 | A | A | A | B |
| A140 | B | B | D | D |
| A141 | A | A | A | A |
| A142 | A | A | A | B |
| A143 | B | B | C | C |
| A144 | A | A | B | B |
| A145 | A | A | D | C |
| A146 | B | B | D | D |
| A147 | A | A | D | C |
| A148 | A | A | D | C |
| A149 | A | A | D | C |
| A150 | A | A | D | D |
| A151 | A | A | D | D |
| A152 | A | A | D | C |
| A153 | A | A | D | C |
| A154 | A | A | D | D |
| A155 | C | B | D | D |
| A156 | C | B | D | D |
| A157 | C | B | D | D |
| A158 | A | A | D | C |
| A159 | A | A | D | C |
| A160 | A | A | D | C |
| A161 | A | A | C | C |
| A162 | A | A | C | C |
| A163 | A | A | C | C |
| A164 | A | A | C | C |
| A165 | A | A | D | D |
| A166 | B | B | D | D |
| A167 | D | D | D | D |
| A168 | D | D | D | D |
| A169 | D | D | D | D |
| A170 | D | D | D | D |
| A171 | A | A | C | C |
| A172 | A | A | D | D |
| A173 | A | A | D | D |
| A174 | A | A | D | D |
| A175 | A | A | D | D |

Note:
DC50: A (<10 nM), B(10-100 nM), C (>100 nM-1000 nM), and D (>1000 nM).
Dₘₐₓ: A (>90% degradation), B (70-90% degradation), C (50-69% degradation), and D (<50% degradation).

### For “B” Series Compounds

The HiBit assay was performed using engineered HT1080 CRISPR knock-in cell utilizing the Nano-Glo HiBiT Lytic Detection System from Promega, Cat #N3040. Compounds were transferred, 25 nL DMSO or test compounds (final DMSO @ 0.1%) to intermediate plates (Corning3570) using ECHO550. Cells were seeded onto compounds at 2000 cells/25 μL/well and incubated for 6 hrs in a tissue culture incubator at 37° C. and 5% CO₂. The LgBiT Protein was diluted at 1:100 and the Nano-Glo® HiBIT Lytic Substrate 1:50 into an appropriate volume of room temperature using the Nano-Glo® HiBiT Lytic Buffer. At the end of compound incubation time, 15 ul of the detection reagent was added to each well (without LgBiT for negative control wells) using the Thermo Scientific multidrop combi. The plate was shaken for 10 mins at RT using Combi. After briefly centrifugation (2000 rpm 1 mins), plate was read on Envision (Ultrasensitive luminescence model). The results of the HiBiT Assay are summarized in Table E3 below.

TABLE E3

| | SMARC A2 | | SMARC A4 | |
|---|---|---|---|---|
| | HT1080 HiBit | | HT1080 HiBit | |
| Compound No. | DC₅₀ (nM) | Dmax (%) | DC50(nM) | Dmax(%) |
| B1 | A | A | A | C |
| B2 | A | A | A | A |
| B3 | A | A | A | A |
| B4 | A | A | A | B |
| B5 | A | A | A | A |
| B6 | A | A | A | A |
| B7 | A | A | A | A |
| B8 | A | A | A | B |
| B9 | A | A | A | A |
| B10 | A | A | A | B |
| B11 | A | A | A | A |
| B12 | A | A | A | A |
| B13 | A | B | B | D |
| B14 | A | A | B | B |
| B15 | A | A | A | A |
| B16 | A | A | A | B |
| B17 | A | A | A | A |
| B18 | A | A | A | B |
| B19 | A | A | B | B |
| B20 | A | A | A | B |
| B21 | A | A | A | A |
| B22 | A | A | B | C |
| B23 | A | A | A | B |
| B24 | A | A | A | A |
| B25 | C | D | B | D |
| B26 | A | A | A | A |
| B27 | A | A | A | B |
| B28 | A | A | A | A |
| B29 | A | A | A | A |
| B30 | A | A | A | A |
| B31 | B | B | B | D |
| B32 | A | A | A | D |
| B33 | D | D | D | D |
| B34 | A | A | A | A |
| B35 | A | A | A | A |
| B36 | A | B | A | D |
| B37 | A | A | A | B |
| B38 | A | A | A | B |
| B39 | A | A | A | A |
| B40 | A | A | B | D |
| B41 | A | A | A | B |
| B42 | A | A | A | B |
| B43 | B | C | D | D |
| B44 | B | D | B | D |
| B45 | B | B | D | D |
| B46 | A | B | A | B |
| B47 | B | B | B | D |
| B48 | A | B | B | D |
| B49 | A | B | D | D |
| B50 | B | B | D | D |
| B51 | A | A | A | B |
| B52 | A | B | D | D |
| B53 | B | B | B | C |
| B54 | B | D | B | D |
| B55 | A | B | B | D |
| B56 | B | D | D | D |
| B57 | B | B | C | D |
| B58 | A | A | A | B |
| B59 | B | C | D | D |
| B60 | A | D | A | D |
| B61 | A | A | A | B |
| B62 | A | C | D | D |
| B63 | A | A | B | D |
| B64 | B | D | D | D |
| B65 | B | D | D | D |
| B66 | A | A | A | A |
| B67 | A | A | A | B |
| B68 | A | A | C | D |
| B69 | A | A | B | D |
| B70 | A | A | A | D |
| B71 | B | C | D | D |
| B72 | A | C | A | D |
| B73 | A | A | B | B |
| B74 | B | D | D | D |
| B75 | B | B | B | D |
| B76 | B | B | B | D |
| B77 | A | A | B | C |
| B78 | A | A | A | A |
| B79 | A | B | B | D |
| B80 | A | A | A | A |
| B81 | A | A | A | D |
| B82 | A | B | A | C |
| B83 | B | D | D | D |
| B84 | A | A | A | A |
| B85 | A | A | A | A |
| B86 | A | A | A | B |
| B87 | B | C | D | D |
| B88 | D | D | D | D |
| B89 | A | A | B | A |
| B90 | A | A | A | A |
| B91 | A | A | A | A |
| B92 | D | D | D | D |
| B93 | B | C | B | D |
| B94 | A | A | A | A |
| B95 | B | C | D | D |
| B96 | A | B | B | D |
| B97 | B | C | B | D |
| B98 | A | B | B | D |
| B99 | A | C | D | D |
| B100 | A | B | A | D |
| B101 | D | D | D | D |
| B102 | A | A | A | B |
| B103 | A | A | A | A |
| B104 | A | A | B | B |
| B105 | A | A | B | B |
| B106 | A | A | A | A |
| B107 | A | A | A | A |
| B108 | B | A | B | C |
| B109 | A | A | B | C |
| B110 | A | A | A | B |
| B111 | A | A | A | C |
| B112 | A | A | A | A |
| B113 | B | A | C | D |
| B114 | A | B | B | D |
| B115 | A | A | A | B |
| B116 | A | B | D | D |
| B117 | A | A | B | C |
| B118 | A | B | C | C |
| B119 | A | A | A | A |
| B120 | A | A | A | B |
| B121 | A | A | A | B |
| B122 | B | B | B | D |
| B123 | A | B | B | D |
| B124 | A | B | B | D |
| B125 | A | A | A | B |
| B126 | A | A | A | C |
| B127 | A | A | A | B |
| B128 | A | A | A | B |
| B129 | A | A | A | B |
| B130 | A | A | B | C |
| B131 | A | A | A | C |
| B132 | A | A | B | B |
| B133 | A | A | A | B |
| B134 | A | A | A | C |
| B135 | A | A | A | B |
| B136 | A | A | A | B |
| B137 | A | A | A | D |
| B138 | A | A | A | A |
| B139 | A | A | A | B |
| B140 | A | A | A | C |
| B141 | A | A | B | D |
| B142 | A | A | A | C |
| B143 | B | B | D | D |
| B144 | B | C | A | C |
| B145 | B | B | B | C |
| B146 | A | A | B | C |
| B147 | B | C | C | C |
| B148 | B | D | A | D |
| B149 | A | A | A | B |
| B150 | A | C | B | D |
| B151 | A | A | B | C |
| B152 | A | A | A | B |
| B153 | A | B | A | B |
| B154 | A | A | A | B |
| B155 | A | A | A | B |
| B156 | A | A | A | C |
| B157 | A | A | A | A |
| B158 | A | A | A | A |
| B159 | A | A | A | A |
| B160 | A | A | A | C |
| B161 | A | A | A | C |
| B162 | A | A | A | C |
| B163 | A | A | A | A |
| B164 | A | A | A | B |
| B165 | B | C | B | C |
| B166 | B | B | B | B |
| B167 | A | B | B | D |
| B168 | A | A | B | B |
| B169 | A | B | A | C |

Note:
DC₅₀: A (<100 nM), B (100-500 nM), C (501-1000 nM), and D (>1000 nM).
Dₘₐₓ: A (>90% degradation), B (70-90% degradation), C (50-69% degradation), and D (<50% degradation).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of Formula II 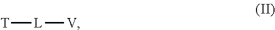
or a pharmaceutically acceptable salt, solvate, or stereoisomer thereof, wherein:
T is of Formula II-1 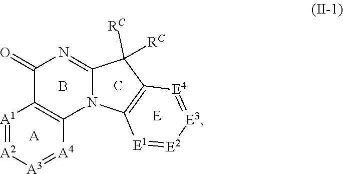
wherein:
A¹ is CR^{A1} or N;
A² is CR^{A2} or N;
A³ is CR^{A3} or N;
A⁴ is CR^{A4} or N;
R^{A1}, R^{A2}, R^{A3}, and R^{A4} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c} S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c} S(═O)₂R^{a}, —NR^{c} S(═O)R^{a}, —NR^{c} S(═O)₂OR^{a}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two R^{C} together form an oxo; or
two R^{C}, together with the carbon atom to which they are attached, form Ring D 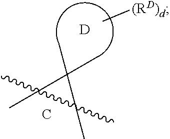
Ring D is C₃₋₁₂ carbocycle or 3- to 12-membered heterocycle;
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c} S(═O)₂R^{a}, —NR^{c} S(═O)R^{a}, —NR^{c} S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 10, as valency permits;
E¹ is CR^{E1} or N;
E² is CR^{E2} or N;
E³ is CR^{E3} or N;
E⁴ is CR^{E4} or N;
R^{E1}, R^{E2}, R^{E3}, and R^{E4} are independently 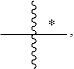
hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c} S(═O)₂R^{a}, —NR^{c} S(═O)R^{a}, —NR^{c} S(═O)₂OR′, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
wherein one of R^{E1}, R^{E2}, R^{E3}, and R^{E4} is 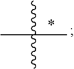
or
one of R^{E2}, R^{E3}, or R^{E4} is 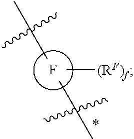 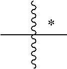
denotes attachment to L;
Ring F is C₃₋₁₂ carbocyclyl, 3- to 12-membered heterocyclyl, C₆₋₁₀ aryl, or 5- to 10-membered heteroaryl;
each R^{F} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c} S(═O)₂R^{a}, —NR^{c} S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
f is an integer selected from 0 to 10, as valency permits,
L is of Formula II-2 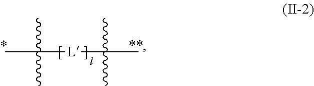
wherein:
* denotes attachment to T and ** denotes attachment to V;
each L′ is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L′})—, —C(═O)O—, —N(R^{L′})—, —O—, —S—, or —S(═O)₂—, wherein the alkylene, heteroalkylene, alkenylene, alkynylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R®, —S(═O)₂OR^{b}, —S(═O)₂NR^{c}R^{d}, —C(═O) R*, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
l is an integer selected from 0 to 5,
V is of Formula II-3-i, II-3-ii, II-3-iii, II-3-iv, II-3-v, II-3-vi, II-3-vii, or II-3-viii 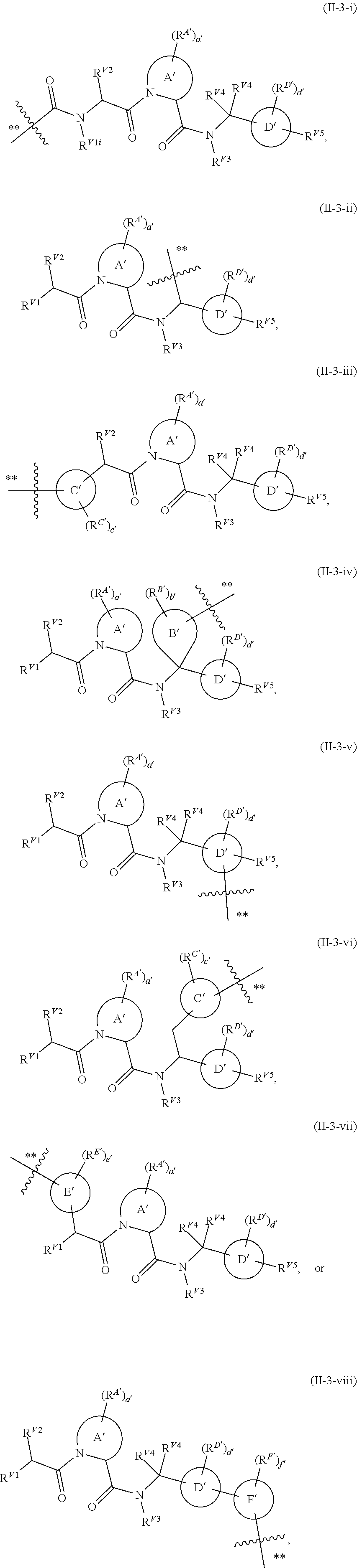
wherein:
** denotes attachment to L;
Ring A′ is 5- to 7-membered heterocycle;
each R^{A′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a′ is an integer selected from 0 to 10, as valency permits;
Ring B′ is C₃₋₆ carbocycle or 3- to 6-membered heterocycle;
each R^{B′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
b′ is an integer selected from 0 to 10, as valency permits;
Ring C′ is C₆ aryl or 5- to 6-membered heteroaryl;
each R^{C′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
c′ is an integer selected from 0 to 5, as valency permits;
Ring D′ is C₆ aryl or 5- to 6-membered heteroaryl;
each R^{D′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
R^{D′} and R^{V4}, together with the intervening atoms, form C₃₋₁₂ carbocyclyl, 3- to 12-membered heterocyclyl, C₆₋₁₀ aryl, or 5- to 10-membered heteroaryl, wherein the carbocyclyl, heterocyclyl, aryl, or heteroaryl is optionally substituted with one or more R^{u};
d′ is an integer selected from 0 to 5, as valency permits; Ring E′ is C₃₋₈ carbocycle or 3- to 8-membered heterocycle;
each R^{E′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
e′ is an integer selected from 0 to 5, as valency permits;
Ring F′ is C₆ aryl or 5- to 6-membered heteroaryl;
each R^{F′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
f′ is an integer selected from 0 to 5, as valency permits;
R^{V1} is —N(R^{V1i})C(═O)R^{V1ii} or 5- to 6-membered heteroaryl optionally substituted with one or more R^{u};
R^{V1i} is hydrogen, C₁₋₆ alkyl, C₃₋₆ carbocyclyl, or 3- to 6-membered heterocyclyl, wherein the alkyl, carbocyclyl, or heterocyclyl is optionally substituted with one or more R^{u};
R^{V1i} is C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{V1iia},
each R^{V1ia} is independently oxo, halogen, —CN, —NO₂, —OH, —NH₂, C₁₋₆ alkyl, —(C₁₋₆ alkylene)-(3- to 12-membered heterocyclyl), C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkylene, alkoxy, alkylamino, alkenyl, alkynyl, carbocyclyl, heterocyclyl, aryl, or heteroaryl is optionally substituted with one or more R^{u};
R^{V2} is hydrogen, C₁₋₆ alkyl, C₃₋₁₂ carbocyclyl, or 3- to 12-membered heterocyclyl, wherein the alkyl, carbocyclyl, heterocyclyl is optionally substituted with one or more R^{u};
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
each R^{V4} is independently hydrogen, C₁₋₆ alkyl, C₂₋₆ alkenyl, C₂₋₆ alkynyl, C₃₋₆ carbocyclyl, or 3- to 6-membered heterocyclyl, wherein the alkyl, alkenyl, alkynyl, carbocyclyl, or heterocyclyl is optionally substituted with one or more R^{V4a}, or
two R^{V4}, together with the carbon atom to which they are attached, form C₃₋₆ carbocycle or 3- to 6-membered heterocycle, wherein the carbocycle or heterocycle is optionally substituted with one or more R^{u};
each R^{V4a} is independently oxo, halogen, —CN, —NO₂, —OH, —NH₂, C₁₋₆ alkyl, C₁₋₁₂ heteroalkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, heteroalkyl, alkoxy, alkylamino, alkenyl, alkynyl, carbocyclyl, heterocyclyl, aryl, or heteroaryl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, or —C(═O)R^{a}, wherein the alkyl, alkoxy, alkylamino, alkenyl, alkynyl, carbocyclyl, heterocyclyl, aryl, or heteroaryl is optionally substituted with one or more R^{u};
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c} S(═O)₂R^{a}, —NR^{c} S(═O)R^{a}, —NR^{c} S(═O)₂OR^{a}, —NR^{c} S(═O)₂NR^{c}R^{d}, —NR^{b}C(═O)NR^{c}R^{d}, —NR^{b}C(═O)R^{a}, —NR^{b}C(═O)OR^{a}, —OS(═O)₂R^{a}, —OS(═O)₂OR, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₃₋₆ carbocyclyl, 3- to 6-membered heterocyclyl, C₆ aryl, or 5- to 6-membered heteroaryl, wherein the carbocyclyl, heterocyclyl, aryl, or heteroaryl is optionally substituted with one or more Rz;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and Rd, together with the nitrogen atom to which they are attached, form 3- to 12-membered heterocyclyl,
wherein each occurrence of R^{a}, Rb, Rc, and R^{d} is independently and optionally substituted with one or more Rz; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

2. The compound of claim 1, wherein T is of Formula II-1-i or II-1-ii 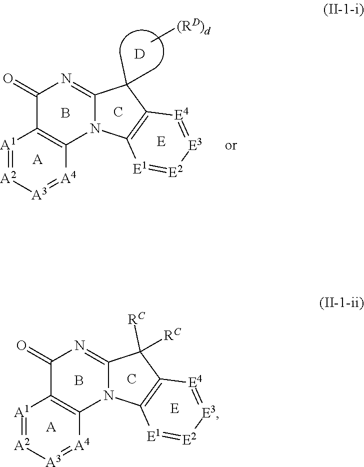
wherein:
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

3. The compound of claim 1, wherein T is of Formula II-1-i-1, II-1-i-2, II-1-i-3, II-1-ii-1, II-1-ii-2, II-1-ii-3, II-1-iii-1, II-1-iii-2, or II-1-iii-3 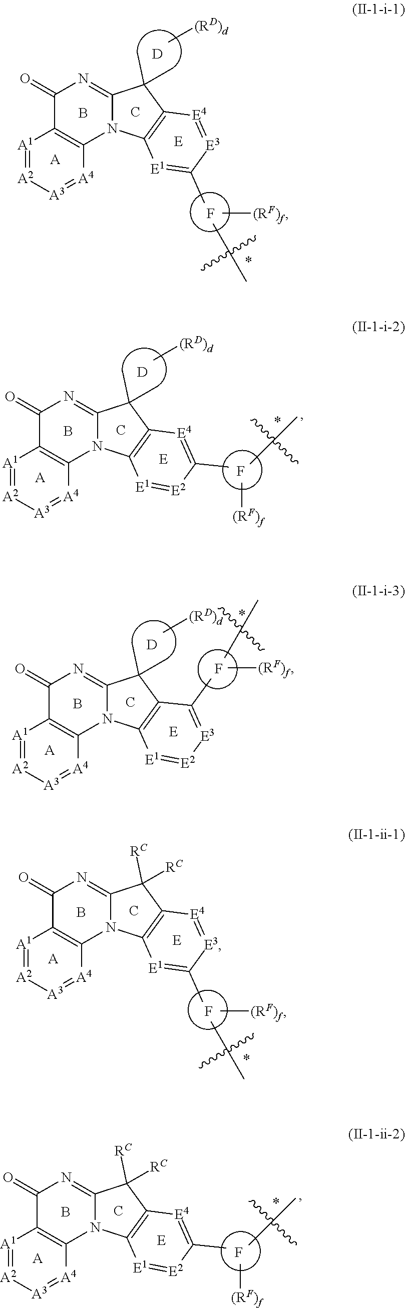 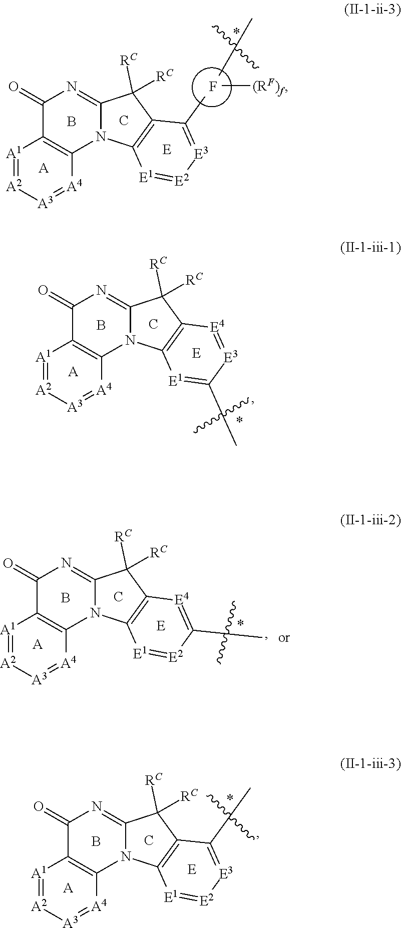
wherein:
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

4. The compound of any one of claims 1-3, wherein R^{A1} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

5. The compound of any one of claims 1-3, wherein RAI is halogen or hydrogen.

6. The compound of claim 1, wherein T is of Formula II-1-i-4, II-1-i-5, II-1-i-6, II-1-ii-4, II-1-ii-5, II-1-ii-6, II-1-iii-4, II-1-iii-5, or II-1-iii-6 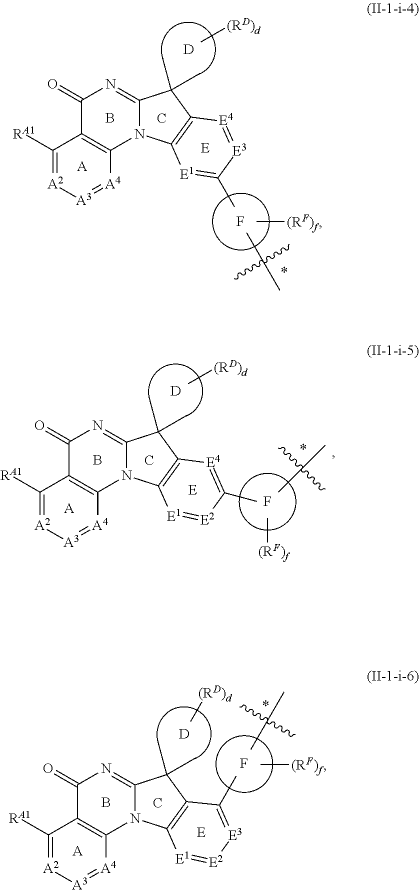 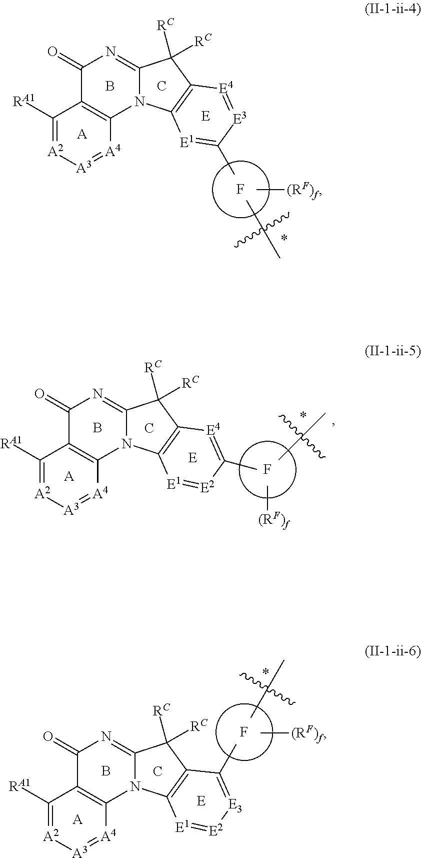 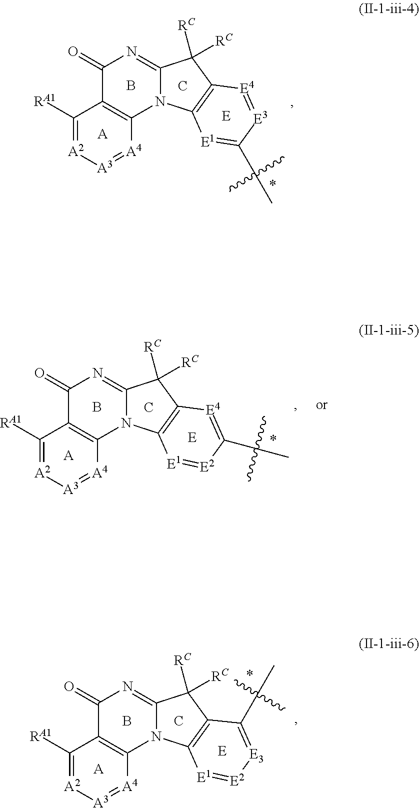
wherein
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; and
R^{A1} is halogen or hydrogen.

7. The compound of any one of claims 1-6, wherein Ring F is 3- to 12-membered heterocyclyl or C₃₋₁₂ carbocyclyl.

8. The compound of any one of claims 1-6, wherein Ring F is 5- to 6-membered heteroaryl.

9. The compound of any one of claims 1-8, wherein each R^{F} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

10. The compound of any one of claims 1-9, wherein f is 0.

11. The compound of any one of claims 1-10, wherein R^{E1}, R^{E2}, and R^{E4}, R^{E1}, R^{E3}, and R^{E4}, or R^{E1}, R^{E2}, and R^{E3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

12. The compound of claim 11, wherein each of R^{E1}, R^{E2}, and R^{E4}, each of REI, R^{E3}, and R^{E4}, or each of R^{E1}, R^{E2}, and R^{E3} is hydrogen.

13. The compound of any one of claims 1-10, wherein one or two of E¹, E², and E⁴, one or two of E¹, E³, and E⁴, or one or two of E¹, E², and E³ are N.

14. The compound of claim 13, wherein one or two of R^{E1}, R^{E2}, and R^{E4}, one or two of REI, R^{E3}, and R^{E4}, or one or two of R^{E1}, R^{E2}, and R^{E3} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

15. The compound of claim 14, wherein one or two of R^{E1}, R^{E2}, and R^{E4}, one or two of R^{E1}, R^{E3}, and R^{E4}, or one or two of R^{E1}, R^{E2}, and R^{E3} are hydrogen.

16. The compound of any one of claims 1-15, wherein each of R^{A2}, R^{A3}, and R^{A4} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

17. The compound of any one of claims 1-15, wherein each of R^{A2}, R^{A3}, and R^{A4} is hydrogen.

18. The compound of any one of claims 1-17, wherein Ring D is C₃₋₁₂ carbocycle.

19. The compound of claim 18, wherein Ring D is C₅₋₇ carbocycle.

20. The compound of any one of claims 1-17, wherein Ring D is 3- to 12-membered heterocycle.

21. The compound of claim 20, wherein Ring D is 5- to 7-membered heterocycle.

22. The compound of any one of claims 1-21, wherein each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

23. The compound of any one of claims 1-22, wherein d is 0.

24. The compound of any one of claims 1-17, wherein each R^{C} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

25. The compound of any one of claims 1-17, wherein each R^{C} is independently C₁₋₆ alkyl optionally substituted with one or more halogen.

26. The compound of any one of claims 1-25, wherein V is of Formula II-3-i

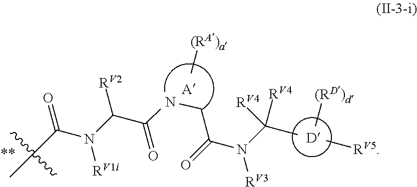

27. The compound of any one of claims 1-25, wherein V is of Formula II-3-ii

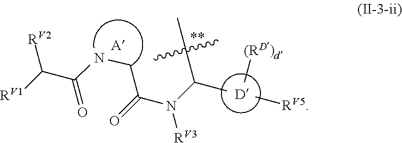

28. The compound of any one of claims 1-25, wherein V is of Formula II-3-iii

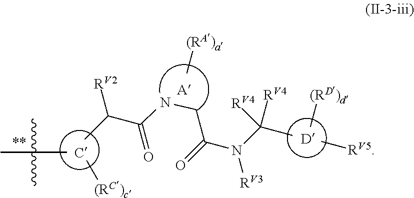

29. The compound of any one of claims 1-25, wherein V is of Formula II-3-v

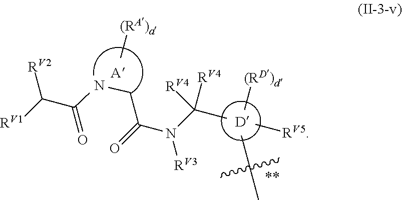

30. The compound of any one of claims 1-25, wherein V is of Formula II-3-vi

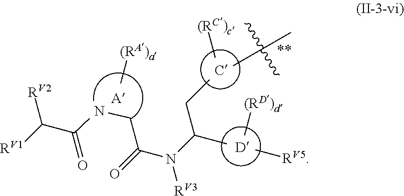

31. The compound of any one of claims 1-25, wherein V is of Formula II-3-vii

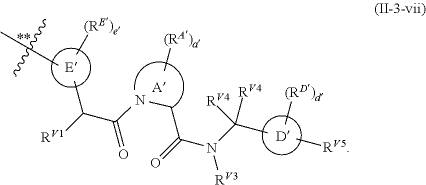

32. The compound of any one of claims 26-31, wherein R^{V1} is —N(R^{V1i})C(═O)R^{V1ii} or 5-membered heteroaryl, wherein R^{V1ii} is C₃₋₆ carbocyclyl optionally substituted with one or more R^{V1ia}, and the heteroaryl is optionally substituted with one or more R^{u}.

33. The compound of claim 32, wherein each R^{V1iia} is independently halogen, —CN, C₁₋₆ alkyl, (C₁₋₆ alkylene)-(3- to 12-membered heterocyclyl), or C₁₋₆ alkylamino, wherein the alkyl, alkylene, or alkylamino is optionally substituted with one or more R^{u}.

34. The compound of any one of claims 26-33, wherein R^{V2} is hydrogen, C₁₋₆ alkyl, C₅₋₆ carbocyclyl, or 5- to 6-membered heterocyclyl.

35. The compound of any one of claims 26-34, wherein each R^{V4} is independently hydrogen or C₁₋₆ alkyl optionally substituted with one or more R^{V4a}.

36. The compound of claim 35, wherein each R^{V4a} is independently —OH, C₁₋₆ heteroalkyl, C₁₋₆ alkylamino, or 3- to 12-membered heterocyclyl, wherein the heteroalkyl, alkylamino, or heterocyclyl is optionally substituted with one or more R^{u}.

37. The compound of any one of claims 26-34, wherein R^{D′} and R^{V4}, together with the atoms to which they are bonded, form C₃₋₁₂ carbocyclyl or 5- to 10-membered heteroaryl, wherein the carbocyclyl or heteroaryl is optionally substituted with one or more R^{u}.

38. The compound of any one of claims 26-37, wherein Ring C′ is 5-membered heteroaryl.

39. The compound of any one of claims 26-38, wherein each R^{C′} is independently halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl, or heterocyclyl is optionally substituted with one or more R^{u}.

40. The compound of any one of claims 26-39, wherein c′ is 0 or 1.

41. The compound of any one of claims 26-40, wherein Ring E′ is 5- to 6-membered heterocycle.

42. The compound of any one of claims 26-41, wherein each R^{E′} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl, or heterocyclyl is optionally substituted with one or more R^{u}.

43. The compound of any one of claims 26-42, wherein e′ is 0.

44. The compound of any one of claims 26-43, wherein Ring F′ is 5- to 6-membered heteroaryl.

45. The compound of any one of claims 26-44, wherein each R^{F′} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl, or heterocyclyl is optionally substituted with one or more R^{u}.

46. The compound of any one of claims 26-45, wherein f′ is 0.

47. The compound of any one of claims 1-46, wherein 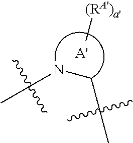
is

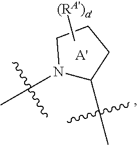

48. The compound of claim 47, wherein a′ is 1 or 2, and at least one of R^{A′} is —OH.

49. The compound of claim 48, wherein 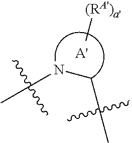
is

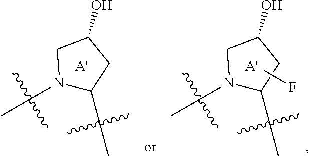

50. The compound of any one of claims 1-49, wherein Ring D′ is phenyl.

51. The compound of claim 50, wherein 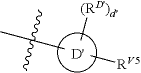
is

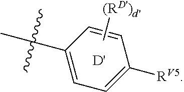

52. The compound of any one of claims 1-51, wherein d′ is 0.

53. The compound of any one of claims 1-52, wherein R^{V3} is hydrogen.

54. The compound of any one of claims 1-53, wherein R^{V5} is halogen, C₂₋₆ alkynyl, C₆₋₁₀ aryl, 5- to 6-membered heteroaryl, or —C(═O)R^{a}, wherein the alkynyl, aryl, or heteroaryl is optionally substituted with one or more R.

55. The compound of any one of claims 1-54, wherein each L′ is independently C₁₋₆ alkylene, C₁₋₆ heteroalkylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L′})—, —C(═O)O—, —N(R^{L′})—, —O—, or —S(═O)₂—, wherein the alkylene, heteroalkylene, carbocyclylene, heterocyclylene, arylene, or heteroarylene is optionally substituted with one or more R^{u}, and l is an integer selected from 1 to 4.

56. The compound of any one of claims 1-53, wherein each L′ is independently C₁₋₆ alkylene, C₁₋₆ heteroalkylene, C₂₋₆ alkynylene, C₃₋₁₂ carbocyclylene, 3- to 12-membered heterocyclylene, —C(═O)—, —C(═O)N(R^{L′})—, —C(═O)O—, —N(R^{L′})—, —O—, or —S(═O)₂—, wherein the alkylene, heteroalkylene, carbocyclylene, or heterocyclylene is optionally substituted with one or more R^{u}, and l is an integer selected from 1 to 4.

57. The compound of any one of claims 1-54, wherein L is absent, C₁₋₁₂ alkylene, C₁₋₆ heteroalkylene, C₃₋₁₂ carbocyclylene, *—C₁₋₁₂ alkylene-O—, *—C₁₋₁₂ alkynylene-O—, *—(C₁₋₁₂ alkylene)-(C₁₋₁₂ alkynylene)-, *—(C₁₋₁₂ alkylene)-O—(C₁₋₁₂ alkynylene)-, *—(C₃₋₁₂ carbocyclylene)-O—, *—(C₁₋₆ alkylene)-(C₃₋₁₂ carbocyclylene)-, *—(C₃₋₁₂ carbocyclylene)-(C₁₋₆ alkylene)-, *—(C₃₋₁₂ carbocyclylene)-(C₁₋₆ alkylene)-O—, *—(C₃₋₁₂ carbocyclylene)-(C₁₋₆ heteroalkylene)-, *—C(═O)—(C₁₋₆ alkylene)-, *—C(═O)—(C₃₋₁₂ carbocyclylene)-, *—C(═O)-(3- to 12-membered heterocyclylene)-, *—(C₁₋₆ alkylene)-(C₃₋₁₂ carbocyclylene)-O—, *-(3- to 12-membered heterocyclylene)-(C₁₋₆ alkylene)-(C₃₋₁₂ carbocyclylene)-, *—(C₁₋₆ alkylene)-(3- to 12-membered heterocyclylene)-, *—(C₁₋₆ alkylene)-(C₃₋₁₂ carbocyclylene)-C(═O)N(R^{L′})—, *-(3- to 12-membered heterocyclylene)-(C₁₋₆ alkylene)-, *—(C₁₋₆ alkylene)-C(═O)-(3- to 12-membered heterocyclylene)-, *—(C₁₋₆ alkylene)-(C₃₋₁₂ carbocyclylene)-C(═O)-(3- to 12-membered heterocyclylene)-, *—(C₃₋₁₂ carbocyclylene)-N(R^{L′})—C(═O)—(C₁₋₆ alkylene)-, *—(C₁₋₆ alkylene)-(C₃₋₁₂ carbocyclylene)-N(R^{L′}) C(═O)—(C₁₋₆ alkylene)-, *-(3- to 12-membered heterocyclylene)-C(═O)—, *—(C₁₋₆ alkylene)-(3- to 12-membered heterocyclylene)-C(═O)—, *—(C₁₋₆ alkylene)-(C₃₋₁₂ carbocyclylene)-C(═O)N(R^{L′})—(C₁₋₆ alkylene)-, *—(C₁₋₆ alkylene)-(C₃₋₁₂ carbocyclylene)-C(═O)—(C₁₋₆ alkylene)-, *-(3- to 12-membered heterocyclylene)-C(═O)—(C₁₋₆ alkylene)-, *—(C₃₋₁₂ carbocyclylene)-N(R^{L′}) C(═O)—(C₁₋₆ alkylene)-O—, *—(C₁₋₆ alkylene)-N(R^{L′}) C(═O)—(C₁₋₆ alkylene)-, *—(C₁₋₆ alkylene)-O—(C₁₋₆ alkylene)-N(R^{L′}) C(═O)—, *—(C₁₋₆ alkylene)-(C₃₋₁₂ carbocyclylene)-(C₁₋₆ alkylene)-, *—(C₁₋₆ alkylene)-(C₃₋₁₂ carbocyclylene)-O—, *—(C₁₋₆ alkylene)-(C₆₋₁₀ arylene)-, *—S(═O)₂—(C₃₋₁₂ carbocyclylene)-, or *—(C₁₋₆ alkylene)-(5- to 10-membered heteroarylene)-, wherein the alkylene, heteroalkylene, heterocyclylene, carbocyclylene, arylene, or heteroarylene is optionally substituted with one or more R^{u}, and *denotes attachment to T.

58. A compound selected from the compounds in Tables 1 and 2, or a pharmaceutically acceptable salt thereof.

59. A pharmaceutical composition comprising the compound of any one of claims 1-58, and a pharmaceutically acceptable excipient.

60. A method of degrading a SMARCA2 or SMARCA4 protein in a patient or biological sample comprising contacting said patient or biological sample with a compound of any one of claims 1-58.

61. Use of a compound of any one of claims 1-58 in the manufacture of a medicament for degrading a SMARCA2 or SMARCA4 protein in a patient or biological sample.

62. A compound of any one of claims 1-58 for use in degrading a SMARCA2 or SMARCA4 protein in a patient or biological sample.

63. A method of treating a disease or disorder comprising administering to a patient in need thereof a compound of any one of claims 1-58.

64. Use of a compound of any one of claims 1-58 in the manufacture of a medicament for treating a disease or disorder.

65. A compound of any one of claims 1-58 for use in treating a disease or disorder.

66. The method, use, or compound for use of any one of claims 63-65, wherein the disease or disorder is a SMARCA2 or SMARCA4 protein-mediated disease or disorder.

67. The method, use, or compound for use of any one of claims 63-65, wherein the disease or disorder is cancer.

68. The method, use, or compound for use of claim 67, wherein the cancer is selected from NSCLC adenocarcinoma (LUAD), NSCL squamous cell carcinoma (LUSC), liver hepatocellular carcinoma (LIHC), uterine corpus endometrial carcinoma (UCEC), esophageal carcinoma (ESCA), skin cutaneous melanoma (SKCM), stomach adenocarcinoma (STAD), colon adenocarcinoma (COAD), bladder urothelial carcinoma (BLCA), and uterine carcinosarcoma (UCS).

69. The method, use, or compound for use of claim 67, wherein the cancer is selected from NSCLC adenocarcinoma (LUAD), NSCL squamous cell carcinoma (LUSC), liver hepatocellular carcinoma (LIHC), and uterine corpus endometrial carcinoma (UCEC).
